SEQUENCE LISTING (843 sequences)

SEQ ID NO: 1 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 2 - protein, 308 aa, Mus musculus
MGVPAVPEASSPRWGTLLLAIFLAASRGLVAAFKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITAAALATGACIVGILCLPLILLLVYKQRQVASHRRAQELVRMDSNTQGIENPGFETTPPFQGMPEAKTRPPLSYVAQRQPSESGRYLLSDPSTPLSPPGPGDVFFPSLDPVPDSPNSEAI

SEQ ID NO: 3 - protein, 309 aa, Mus musculus
MGVPAVPEASSPRWGTLLLAIFLAASRGLVAAFKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITAAALATGACIVGILCLPLILLLVYKQRQVASHRRAQELVRMDSSNTQGIENPGFETTPPFQGMPEAKTRPPLSYVAQRQPSESGRYLLSDPSTPLSPPGPGDVFFPSLDPVPDSPNSEAI

SEQ ID NO: 4 - DNA, 4774 bp, Homo sapiens
gggggcgggtgcctggagcacggcgctggggccgcccgcagcgctcactcgctcgcactcagtcgcgggaggcttccccgcgccggccgcgtcccgcccgctccccggcaccagaagttcctctgcgcgtccgacggcgacatgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctagcccagctgggggacagtgggctgttgtggctgggtctggggcaggtgcatttgagccagggctggctctgtgagtggcctccttggcctcggccctggttccctccctcctgctctgggctcagatactgtgacatcccagaagcccagcccctcaacccctctggatgctacatggggatgctggacggctcagcccctgttccaaggattttggggtgctgagattctcccctagagacctgaaattcaccagctacagatgccaaatgacttacatcttaagaagtctcagaacgtccagcccttcagcagctctcgttctgagacatgagccttgggatgtggcagcatcagtgggacaagatggacactgggccaccctcccaggcaccagacacagggcacggtggagagacttctcccccgtggccgccttggctcccccgttttgcccgaggctgctcttctgtcagacttcctctttgtaccacagtggctctggggccaggcctgcctgcccactggccatcgccaccttccccagctgcctcctaccagcagtttctctgaagatctgtcaacaggttaagtcaatctggggcttccactgcctgcattccagtccccagagcttggtggtcccgaaacgggaagtacatattggggcatggtggcctccgtgagcaaatggtgtcttgggcaatctgaggccaggacagatgttgccccacccactggagatggtgctgagggaggtgggtggggccttctgggaaggtgagtggagaggggcacctgccccccgccctccccatcccctactcccactgctcagcgcgggccattgcaagggtgccacacaatgtcttgtccaccctgggacacttctgagtatgaagcgggatgctattaaaaactacatggggaaacaggtgcaaaccctggagatggattgtaagagccagtttaaatctgcactctgctgctcctcccccacccccaccttccactccatacaatctgggcctggtggagtcttcgcttcagagccattcggccaggtgcgggtgatgttcccatctcctgcttgtgggcatgccctggctttgtttttatacacataggcaaggtgagtcctctgtggaattgtgattgaaggattttaaagcaggggaggagagtagggggcatctctgtacactctgggggtaaaacagggaaggcagtgcctgagcatggggacaggtgaggtggggctgggcagaccccctgtagcgtttagcaggatgggggccccaggtactgtggagagcatagtccagcctgggcatttgtctcctagcagcctacactggctctgctgagctgggcctgggtgctgaaagccaggatttggggctaggcgggaagatgttcgcccaattgcttggggggttggggggatggaaaaggggagcacctctaggctgcctggcagcagtgagccctgggcctgtggctacagccagggaaccccacctggacacatggccctgcttctaagccccccagttaggcccaaaggaatggtccactgagggcctcctgctctgcctgggctgggccaggggctttgaggagagggtaaacataggcccggagatggggctgacacctcgagtggccagaatatgcccaaaccccggcttctcccttgtccctaggcagaggggggtcccttcttttgttccctctggtcaccacaatgcttgatgccagctgccataggaagagggtgctggctggccatggtggcacacacctgtcctcccagcactttgcagggctgaggtggaaggaccgcttaagcccaggtgttcaaggctgctgtgagctgtgttcgagccactacactccagcctggggacggagcaaaactttgcctcaaaacaaattttaaaaagaaagaaagaaggaaagagggtatgtttttcacaattcatgggggcctgcatggcaggagtggggacaggacacctgctgttcctggagtcgaaggacaagcccacagcccagattccggttctcccaactcaggaagagcatgccctgccctctggggaggctggcctggccccagccctcagctgctgaccttgaggcagagacaacttctaagaatttggctgccagaccccaggcctggctgctgctgtgtggagagggaggcggcccgcagcagaacagccaccgcacttcctcctcagcttcctctggtgcggccctgccctctcttctctggacccttttacaactgaacgcatctgggcttcgtggtttcctgttttcagcgaaatttactctgagctcccagttccatcttcatccatggccacaggccctgcctacaacgcactagggacgtccctccctgctgctgctggggaggggcaggctgctggagccgccctctgagttgcccgggatggtagtgcctctgatgccagccctggtggctgtgggctggggtgcatgggagagctgggtgcgagaacatggcgcctccagggggcgggaggagcactaggggctggggcaggaggctcctggagcgctggattcgtggcacagtctgaggccctgagagggaaatccatgcttttaagaactaattcattgttaggagatcaatcaggaattaggggccatcttacctatctcctgacattcacagtttaatagagacttcctgcctttattccctcccagggagaggctgaaggaatggaattgaaagcaccatttggagggttttgctgacacagcggggactgctcagcactccctaaaaacacaccatggaggccactggtgactgctggtgggcaggctggccctgcctgggggagtccgtggcgatgggcgctggggtggaggtgcaggagccccaggacctgcttttcaaaagacttctgcctgaccagagctcccactacatgcagtggcccagggcagaggggctgatacatggcctttttcagggggtgctcctcgcggggtggacttgggagtgtgcagtgggacagggggctgcaggggtcctgccaccaccgagcaccaacttggcccctggggtcctgcctcatgaatgaggccttccccagggctggcctgactgtgctgggggctgggttaacgttttctcagggaaccacaatgcacgaaagaggaactggggttgctaaccaggatgctgggaacaaaggcctcttgaagcccagccacagcccagctgagcatgaggcccagcccatagacggcacaggccacctggcccattccctgggcattccctgctttgcattgctgcttctcttcaccccatggaggctatgtcaccctaactatcctggaatgtgttgagagggattctgaatgatcaatatagcttggtgagacagtgccgagatagatagccatgtctgccttgggcacgggagagggaagtggcagcatgcatgctgtttcttggccttttctgttagaatacttggtgctttccaacacactttcacatgtgttgtaacttgtttgatccacccccttccctgaaaatcctgggaggttttattgctgccatttaacacagagggcaatagaggttctgaaaggtctgtgtcttgtcaaaacaagtaaacggtggaactacgactaaa

SEQ ID NO: 5 - DNA, 1055 bp, Homo sapiens
ctcgccgcgctgagccgcctcgggacggagccatgcggcgctgggcctgggccgcggtcgtggtccccctcgggccgcagctcgtgctcctcgggggcgtcggggcccggcgggaggcacagaggacgcagcagcctggccagcgcgcagatccccccaacgccaccgccagcgcgtcctcccgcgaggggctgcccgaggcccccaagccatcccaggcctcaggacctgagttctccgacgcccacatgacatggctgaactttgtccggcggccggacgacggcgccttaaggaagcggtgcggaagcagggacaagaagccgcgggatctcttcggtcccccaggacctccaggtgcagaagtgaccgcggagactctgcttcacgagtttcaggagctgctgaaagaggccacggagcgccggttctcagggcttctggacccgctgctgccccagggggcgggcctgcggctggtgggcgaggcctttcactgccggctgcagggtccccgccgggtggacaagcggacgctggtggagctgcatggtttccaggctcctgctgcccaaggtgccttcctgcgaggctccggtctgagcctggcctcgggtcggttcacggcccccgtgtccggcatcttccagttctctgccagtctgcacgtggaccacagtgagctgcagggcaaggcccggctgcgggcccgggacgtggtgtgtgttctcatctgtattgagtccctgtgccagcgccacacgtgcctggaggccgtctcaggcctggagagcaacagcagggtcttcacgctacaggtgcaggggctgctgcagctgcaggctggacagtacgcttctgtgtttgtggacaatggctccggggccgtcctcaccatccaggcgggctccagcttctccgggctgctcctgggcacgtgagggcgcccaggggggctggcgaggagctgccgccggatcccggggaccctcctactgatgcccgtggtcaccacaataaagagccctccaccctcaaaaaaaaaaaaaaaaaaa

SEQ ID NO: 6 - DNA, 1055 bp, Mus musculus; identical to SEQ ID NO: 5

SEQ ID NO: 7-9 - skipped (3 entries)

SEQ ID NO: 10 - protein, 120 aa, Artificial Sequence
  VSTB92 VH
QVQLVQSGAEVKKPGASVKVSCKASGYTFANYLIGWVRQAPGQRLEWMGDIYPGGGFISYNEKFKGRVTITRDTSASTAYMELSSLRSEDTAVYYCARRFDYGGYFFDYWGQGTLVTVSS

SEQ ID NO: 11 - protein, 112 aa, Artificial Sequence
  VSTB92_VL
DIVMTQSPLSLPVTPGEPASISCRSSQSIVHSNGNIYLEWYLQKPGQSPQLLIYKVSNRFSGVPDRFSGSGSGTDFTLKISRVEAEDVGVYYCFQGSHVPWTFGQGTKLEIK

SEQ ID NO: 12 - protein, 330 aa, Artificial Sequence
  IgG1_LALA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 330 aa, Artificial Sequence
  IgG1_INXLALA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 326 aa, Artificial Sequence
  IgG2_sigma
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 107 aa, Artificial Sequence [sequence omitted]

SEQ ID NO: 16-99 - skipped (84 entries)

SEQ ID NO: 100 - protein, 8 aa, Mus musculus
GYTLTDYN

SEQ ID NO: 101 - protein, 8 aa, Mus musculus
INLNYAIT

SEQ ID NO: 102 - protein, 14 aa, Mus musculus
ARGGYRYTYYAMDY

SEQ ID NO: 103 - protein, 6 aa, Mus musculus
ENIYSN

SEQ ID NO: 104 - protein, 3 aa, Mus musculus
AAT

SEQ ID NO: 105 - protein, 9 aa, Mus musculus
QHFWGTPRT

SEQ ID NO: 106 - protein, 121 aa, Mus musculus
EVQLQQFGAELVKPGASVKISCKASGYTLTDYNMDWVKQSHGKSLEWIGHINLNYAITTYNQKFKGKATLTVDKSSSTAYMELRSLTSEDTAVYYCARGGYRYTYYAMDYWGQGTSVTVSS

SEQ ID NO: 107 - protein, 222 aa, Mus musculus
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKLNPKS

SEQ ID NO: 108 - protein, 107 aa, Mus musculus
DIQMTQSPASLSVSVGETVTITCRASENIYSNLAWYQQKQGKSPQLLVYAATNLADGVPSRFSGSGSGTQYSLKINSLQSEDFGSYYCQHFWGTPRTFGGGTKLEIK

SEQ ID NO: 109 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 110 - protein, 8 aa, Homo sapiens
GYTLTDYN

SEQ ID NO: 111 - protein, 8 aa, Homo sapiens
INLNYAIT

SEQ ID NO: 112 - protein, 14 aa, Homo sapiens
ARGGYRYTYYAMDY

SEQ ID NO: 113 - protein, 6 aa, Homo sapiens
ENIYSN

SEQ ID NO: 114 - protein, 3 aa, Homo sapiens
AAT

SEQ ID NO: 115 - protein, 9 aa, Homo sapiens
QHFWGTPRT

SEQ ID NO: 116 - protein, 121 aa, Homo sapiens; identical to SEQ ID NO: 106

SEQ ID NO: 117 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 118 - protein, 107 aa, Homo sapiens; identical to SEQ ID NO: 108

SEQ ID NO: 119 - protein, 107 aa, Homo sapiens [sequence omitted]

SEQ ID NO: 120 - protein, 8 aa, Mus musculus
GYSFTGYT

SEQ ID NO: 121 - protein, 8 aa, Mus musculus
INPYNGGI

SEQ ID NO: 122 - protein, 13 aa, Mus musculus
ARRTLLRPYFFDY

SEQ ID NO: 123 - protein, 10 aa, Mus musculus
QSVSTSTFSY

SEQ ID NO: 124 - protein, 3 aa, Mus musculus
YAS

SEQ ID NO: 125 - protein, 9 aa, Mus musculus
QHSWEIPYT

SEQ ID NO: 126 - protein, 120 aa, Mus musculus
EVQLQQSGPELVKPGTSMKISCKASGYSFTGYTMNWVRQSHGKNLEWIGLINPYNGGINYNQKFKARATLTVDKSSSTAYMELLSLTSEDSAVYYCARRTLLRPYFFDYWGQGTTLTVSS

SEQ ID NO: 127 - protein, 120 aa, Mus musculus; identical to SEQ ID NO: 126

SEQ ID NO: 128 - protein, 111 aa, Mus musculus
DIVLTQSPASLAVSLGQRATISCRASQSVSTSTFSYMHWYQQKPGQPPKLLIKYASNLESGVPARFSGSGSGTDFTLNIHPVEEEDTATYYCQHSWEIPYTFGGGTKLEIK

SEQ ID NO: 129 - protein, 111 aa, Mus musculus; identical to SEQ ID NO: 128

SEQ ID NO: 130 - protein, 8 aa, Homo sapiens
GYSFTGYT

SEQ ID NO: 131 - protein, 8 aa, Homo sapiens
INPYNGGI

SEQ ID NO: 132 - protein, 13 aa, Homo sapiens
ARRTLLRPYFFDY

SEQ ID NO: 133 - protein, 10 aa, Homo sapiens
QSVSTSTFSY

SEQ ID NO: 134 - protein, 3 aa, Homo sapiens
YAS

SEQ ID NO: 135 - protein, 9 aa, Homo sapiens
QHSWEIPYT

SEQ ID NO: 136 - protein, 120 aa, Homo sapiens; identical to SEQ ID NO: 126

SEQ ID NO: 137 - protein, 326 aa, Homo sapiens; identical to SEQ ID NO: 117

SEQ ID NO: 138 - protein, 111 aa, Homo sapiens; identical to SEQ ID NO: 128

SEQ ID NO: 139 - protein, 107 aa, Homo sapiens [sequence omitted]

SEQ ID NO: 140 - protein, 8 aa, Mus musculus
GFDFSRYW

SEQ ID NO: 141 - protein, 8 aa, Mus musculus
VYPDSSTI

SEQ ID NO: 142 - protein, 7 aa, Mus musculus
ARGRGDY

SEQ ID NO: 143 - protein, 6 aa, Mus musculus
GNIHNY

SEQ ID NO: 144 - protein, 3 aa, Mus musculus
NAK

SEQ ID NO: 145 - protein, 9 aa, Mus musculus
QNFWSTPFT

SEQ ID NO: 146 - protein, 114 aa, Mus musculus
EVKLLESGGGLVQPGGSLKLSCAASGFDFSRYWMSWVRQAPGKGLEWIGEVYPDSSTINYTPSLKDKFIISRDNAKNTLYLQMIKVRSEDTALYYCARGRGDYWGQGTSVTVSS

SEQ ID NO: 147 - protein, 326 aa, Mus musculus; identical to SEQ ID NO: 117

SEQ ID NO: 148 - protein, 107 aa, Mus musculus
DIQMTQSPASLSASVGETVTITCRASGNIHNYLSWYHQKQGKSPQLLVYNAKTLADGVPSRFSGSGSGTQYSLKINSLQPEDFGSYYCQNFWSTPFTFGSGTKLEIK

SEQ ID NO: 149 - protein, 107 aa, Mus musculus [sequence omitted]

SEQ ID NO: 150 - protein, 8 aa, Homo sapiens
GFDFSRYW

SEQ ID NO: 151 - protein, 8 aa, Homo sapiens
VYPDSSTI

SEQ ID NO: 152 - protein, 7 aa, Homo sapiens
ARGRGDY

SEQ ID NO: 153 - protein, 6 aa, Homo sapiens
GNIHNY

SEQ ID NO: 154 - protein, 3 aa, Homo sapiens
NAK

SEQ ID NO: 155 - protein, 9 aa, Homo sapiens
QNFWSTPFT

SEQ ID NO: 156 - protein, 114 aa, Homo sapiens; identical to SEQ ID NO: 146

SEQ ID NO: 157 - protein, 326 aa, Homo sapiens; identical to SEQ ID NO: 117

SEQ ID NO: 158 - protein, 107 aa, Homo sapiens; identical to SEQ ID NO: 148

SEQ ID NO: 159 - protein, 107 aa, Homo sapiens [sequence omitted]

SEQ ID NO: 160 - protein, 8 aa, Homo sapiens
GFDFSRYW

SEQ ID NO: 161 - protein, 8 aa, Homo sapiens
VYPDSSTI

SEQ ID NO: 162 - protein, 7 aa, Homo sapiens
ARGRGDY

SEQ ID NO: 163 - protein, 6 aa, Homo sapiens
GNIHNY

SEQ ID NO: 164 - protein, 3 aa, Homo sapiens
NAK

SEQ ID NO: 165 - protein, 9 aa, Homo sapiens
QNFWSTPFT

SEQ ID NO: 166 - protein, 114 aa, Homo sapiens; identical to SEQ ID NO: 146

SEQ ID NO: 167 - protein, 326 aa, Homo sapiens
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 168 - protein, 107 aa, Homo sapiens; identical to SEQ ID NO: 148

SEQ ID NO: 169 - protein, 107 aa, Homo sapiens [sequence omitted]

SEQ ID NO: 170 - protein, 8 aa, Homo sapiens
GYSFTSYW

SEQ ID NO: 171 - protein, 8 aa, Homo sapiens
IYPGDSDT

SEQ ID NO: 172 - protein, 16 aa, Homo sapiens
ARDVSSFYGYSPMFDY

SEQ ID NO: 173 - protein, 6 aa, Homo sapiens
QSVSSY

SEQ ID NO: 174 - protein, 3 aa, Homo sapiens
DAS

SEQ ID NO: 175 - protein, 9 aa, Homo sapiens
QQRSNWPLT

SEQ ID NO: 176 - protein, 123 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDVSSFYGYSPMFDYWGQGTLVTVSS

SEQ ID NO: 177 - protein, 330 aa, Homo sapiens [sequence omitted]

SEQ ID NO: 178 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 179 - protein, 107 aa, Homo sapiens [sequence omitted]

SEQ ID NO: 180 - protein, 8 aa, Homo sapiens
GYSFTSYW

SEQ ID NO: 181 - protein, 8 aa, Homo sapiens
IYPGDSDT

SEQ ID NO: 182 - protein, 16 aa, Homo sapiens
ARDAHSFYGYSALLDY

SEQ ID NO: 183 - protein, 7 aa, Homo sapiens
QSVSSSY

SEQ ID NO: 184 - protein, 3 aa, Homo sapiens
GAS

SEQ ID NO: 185 - protein, 9 aa, Homo sapiens
QQYGSSPLT

SEQ ID NO: 186 - protein, 123 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDAHSFYGYSALLDYWGQGTLVTVSS

SEQ ID NO: 187 - protein, 330 aa, Homo sapiens [sequence omitted]

SEQ ID NO: 188 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 189 - protein, 107 aa, Homo sapiens [sequence omitted]

SEQ ID NO: 190 - protein, 8 aa, Homo sapiens
GYSFTSYW

SEQ ID NO: 191 - protein, 8 aa, Homo sapiens
IYPGDSDT

SEQ ID NO: 192 - protein, 16 aa, Homo sapiens
ARDSYSFYGHTPVLDY

SEQ ID NO: 193 - protein, 7 aa, Homo sapiens
QSVSSSY

SEQ ID NO: 194 - protein, 3 aa, Homo sapiens
GAS

SEQ ID NO: 195 - protein, 9 aa, Homo sapiens
QQYGSSPLT

SEQ ID NO: 196 - protein, 123 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDSYSFYGHTPVLDYWGQGTLVTVSS

SEQ ID NO: 197 - protein, 330 aa, Homo sapiens [sequence omitted]

SEQ ID NO: 198 - protein, 108 aa, Homo sapiens; identical to SEQ ID NO: 188

SEQ ID NO: 199 - protein, 107 aa, Homo sapiens [sequence omitted]

SEQ ID NO: 200 - protein, 8 aa, Homo sapiens
GYSFTSYW

SEQ ID NO: 201 - protein, 8 aa, Homo sapiens
IYPGDSDT

SEQ ID NO: 202 - protein, 14 aa, Homo sapiens
ARDDALYGGYYLDY

SEQ ID NO: 203 - protein, 12 aa, Homo sapiens
QSVLYSSNNKNY

SEQ ID NO: 204 - protein, 3 aa, Homo sapiens
WAS

SEQ ID NO: 205 - protein, 9 aa, Homo sapiens
QQYYSTPLT

SEQ ID NO: 206 - protein, 121 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDDALYGGYYLDYWGQGTLVTVSS

SEQ ID NO: 207 - protein, 330 aa, Homo sapiens [sequence omitted]

SEQ ID NO: 208 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGQGTKVEIK

SEQ ID NO: 209 - protein, 107 aa, Homo sapiens [sequence omitted]

SEQ ID NO: 210 - protein, 8 aa, Homo sapiens
GYSFTSYW

SEQ ID NO: 211 - protein, 8 aa, Homo sapiens
IYPGDSDT

SEQ ID NO: 212 - protein, 16 aa, Homo sapiens
ARDANSFYSAASIFDY

SEQ ID NO: 213 - protein, 12 aa, Homo sapiens
QSVLYSSNNKNY

SEQ ID NO: 214 - protein, 3 aa, Homo sapiens
WAS

SEQ ID NO: 215 - protein, 9 aa, Homo sapiens
QQYYSTPLT

SEQ ID NO: 216 - protein, 123 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDANSFYSAASIFDYWGQGTLVTVSS

SEQ ID NO: 217 - protein, 330 aa, Homo sapiens [sequence omitted]

SEQ ID NO: 218 - protein, 113 aa, Homo sapiens; identical to SEQ ID NO: 208

SEQ ID NO: 219 - protein, 107 aa, Homo sapiens [sequence omitted]

SEQ ID NO: 220 - protein, 8 aa, Homo sapiens
GGTFSSYA

SEQ ID NO: 221 - protein, 8 aa, Homo sapiens
IIPIFGTA

SEQ ID NO: 222 - protein, 13 aa, Homo sapiens
ARSSYGWSYEFDY

SEQ ID NO: 223 - protein, 6 aa, Homo sapiens
QSIATN

SEQ ID NO: 224 - protein, 3 aa, Homo sapiens
AAS

SEQ ID NO: 225 - protein, 9 aa, Homo sapiens
QQNDDRPIT

SEQ ID NO: 226 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSSYGWSYEFDYWGQGTLVTVSS

SEQ ID NO: 227 - protein, 330 aa, Homo sapiens [sequence omitted]

SEQ ID NO: 228 - protein, 107 aa, Homo sapiens
DIQMTQSPSSLSASVGDRVTITCRASQSIATNLNWYQQKPGKAPKLLIYAASSLQSGVPSRFSGSGSGTDFTLTISSLQPEDFATYYCQQNDDRPITFGQGTKVEIK

SEQ ID NO: 229 - protein, 107 aa, Homo sapiens [sequence omitted]

SEQ ID NO: 230 - protein, 8 aa, Homo sapiens
GGTFSSYA

SEQ ID NO: 231 - protein, 8 aa, Homo sapiens
IIPIFGTA

SEQ ID NO: 232 - protein, 13 aa, Homo sapiens
ARSSYGWSYEFDY

SEQ ID NO: 233 - protein, 6 aa, Homo sapiens
QSIRTD

SEQ ID NO: 234 - protein, 3 aa, Homo sapiens
SAS

SEQ ID NO: 235 - protein, 9 aa, Homo sapiens
QQNERTPIT

SEQ ID NO: 236 - protein, 120 aa, Homo sapiens; identical to SEQ ID NO: 226

SEQ ID NO: 237 - protein, 330 aa, Homo sapiens [sequence omitted]

SEQ ID NO: 238 - protein, 107 aa, Homo sapiens
DIQMTQSPSSLSASVGDRVTITCRASQSIRTDLNWYQQKPGKAPKLLIYSASSLQSGVPSRFSGSGSGTDFTLTISSLQPEDFATYYCQQNERTPITFGQGTKVEIK

SEQ ID NO: 239 - protein, 107 aa, Homo sapiens [sequence omitted]

SEQ ID NO: 240 - protein, 8 aa, Homo sapiens
GGTFSSYA

SEQ ID NO: 241 - protein, 8 aa, Homo sapiens
IIPIFGTA

SEQ ID NO: 242 - protein, 13 aa, Homo sapiens
ARSSYGWSYEFDY

SEQ ID NO: 243 - protein, 6 aa, Homo sapiens
QSINND

SEQ ID NO: 244 - protein, 3 aa, Homo sapiens
AAS

SEQ ID NO: 245 - protein, 9 aa, Homo sapiens
QQNRATPIT

SEQ ID NO: 246 - protein, 120 aa, Homo sapiens; identical to SEQ ID NO: 226

SEQ ID NO: 247 - protein, 330 aa, Homo sapiens [sequence omitted]

SEQ ID NO: 248 - protein, 107 aa, Homo sapiens
DIQMTQSPSSLSASVGDRVTITCRASQSINNDLNWYQQKPGKAPKLLIYAASSLQSGVPSRFSGSGSGTDFTLTISSLQPEDFATYYCQQNRATPITFGQGTKVEIK

SEQ ID NO: 249 - protein, 107 aa, Homo sapiens [sequence omitted]

SEQ ID NO: 250 - protein, 8 aa, Homo sapiens
GGTFSSYA

SEQ ID NO: 251 - protein, 8 aa, Homo sapiens
IIPIFGTA

SEQ ID NO: 252 - protein, 13 aa, Homo sapiens
ARNTFGWSGELDY

SEQ ID NO: 253 - protein, 6 aa, Homo sapiens
QSISNR

SEQ ID NO: 254 - protein, 3 aa, Homo sapiens
SAS

SEQ ID NO: 255 - protein, 9 aa, Homo sapiens
QQNHDNPIT

SEQ ID NO: 256 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARNTFGWSGELDYWGQGTLVTVSS

SEQ ID NO: 257 - protein, 330 aa, Homo sapiens [sequence omitted]

SEQ ID NO: 258 - protein, 107 aa, Homo sapiens
DIQMTQSPSSLSASVGDRVTITCRASQSISNRLNWYQQKPGKAPKLLIYSASSLQSGVPSRFSGSGSGTDFTLTISSLQPEDFATYYCQQNHDNPITFGQGTKVEIK

SEQ ID NO: 259 - protein, 107 aa, Homo sapiens [sequence omitted]

SEQ ID NO: 260 - protein, 8 aa, Homo sapiens
GGTFSSYA

SEQ ID NO: 261 - protein, 8 aa, Homo sapiens
IIPIFGTA

SEQ ID NO: 262 - protein, 13 aa, Homo sapiens
ARSSYGWSYEFDY

SEQ ID NO: 263 - protein, 6 aa, Homo sapiens
QSIATY

SEQ ID NO: 264 - protein, 3 aa, Homo sapiens
AAS

SEQ ID NO: 265 - protein, 9 aa, Homo sapiens
QQNHNRPIT

SEQ ID NO: 266 - protein, 120 aa, Homo sapiens; identical to SEQ ID NO: 226

SEQ ID NO: 267 - protein, 330 aa, Homo sapiens [sequence omitted]

SEQ ID NO: 268 - protein, 107 aa, Homo sapiens
DIQMTQSPSSLSASVGDRVTITCRASQSIATYLNWYQQKPGKAPKLLIYAASSLQSGVPSRFSGSGSGTDFTLTISSLQPEDFATYYCQQNHNRPITFGQGTKVEIK

SEQ ID NO: 269 - protein, 107 aa, Homo sapiens [sequence omitted]

SEQ ID NO: 270 - protein, 8 aa, Homo sapiens
GGTFSSYA

SEQ ID NO: 271 - protein, 8 aa, Homo sapiens
IIPIFGTA

SEQ ID NO: 272 - protein, 13 aa, Homo sapiens
ARHSIGWVAELDY

SEQ ID NO: 273 - protein, 6 aa, Homo sapiens
QSINTD

SEQ ID NO: 274 - protein, 3 aa, Homo sapiens
AAS

SEQ ID NO: 275 - protein, 9 aa, Homo sapiens
QQGASDPIT

SEQ ID NO: 276 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHSIGWVAELDYWGQGTLVTVSS

SEQ ID NO: 277 - protein, 330 aa, Homo sapiens [sequence omitted]

SEQ ID NO: 278 - protein, 107 aa, Homo sapiens
DIQMTQSPSSLSASVGDRVTITCRASQSINTDLNWYQQKPGKAPKLLIYAASSLQSGVPSRFSGSGSGTDFTLTISSLQPEDFATYYCQQGASDPITFGQGTKVEIK

SEQ ID NO: 279 - protein, 107 aa, Homo sapiens [sequence omitted]

SEQ ID NO: 280 - protein, 8 aa, Homo sapiens
GGTFSSYA

SEQ ID NO: 281 - protein, 8 aa, Homo sapiens
IIPIFGTA

SEQ ID NO: 282 - protein, 13 aa, Homo sapiens
ARSSYGWSYEFDY

SEQ ID NO: 283 - protein, 6 aa, Homo sapiens
QSINTD

SEQ ID NO: 284 - protein, 3 aa, Homo sapiens
AAS

SEQ ID NO: 285 - protein, 9 aa, Homo sapiens
QQNRGSPIT

SEQ ID NO: 286 - protein, 120 aa, Homo sapiens; identical to SEQ ID NO: 226

SEQ ID NO: 287 - protein, 330 aa, Homo sapiens [sequence omitted]

SEQ ID NO: 288 - protein, 107 aa, Homo sapiens
DIQMTQSPSSLSASVGDRVTITCRASQSINTDLNWYQQKPGKAPKLLIYAASSLQSGVPSRFSGSGSGTDFTLTISSLQPEDFATYYCQQNRGSPITFGQGTKVEIK

SEQ ID NO: 289 - protein, 107 aa, Homo sapiens [sequence omitted]

SEQ ID NO: 290 - protein, 8 aa, Homo sapiens
GGTFSSYA

SEQ ID NO: 291 - protein, 8 aa, Homo sapiens
IIPIFGTA

SEQ ID NO: 292 - protein, 13 aa, Homo sapiens
ARHSIGWVAELDY

SEQ ID NO: 293 - protein, 6 aa, Homo sapiens
QSIATD

SEQ ID NO: 294 - protein, 3 aa, Homo sapiens
AAS

SEQ ID NO: 295 - protein, 9 aa, Homo sapiens
QQAHWYPLT

SEQ ID NO: 296 - protein, 120 aa, Homo sapiens; identical to SEQ ID NO: 276

SEQ ID NO: 297 - protein, 330 aa, Homo sapiens [sequence omitted]

SEQ ID NO: 298 - protein, 107 aa, Homo sapiens
DIQMTQSPSSLSASVGDRVTITCRASQSIATDLNWYQQKPGKAPKLLIYAASSLQSGVPSRFSGSGSGTDFTLTISSLQPEDFATYYCQQAHWYPLTFGQGTKVEIK

SEQ ID NO: 299 - protein, 107 aa, Homo sapiens [sequence omitted]

SEQ ID NO: 300 - protein, 8 aa, Homo sapiens
GGTFSSYA

SEQ ID NO: 301 - protein, 8 aa, Homo sapiens
IIPIFGTA

SEQ ID NO: 302 - protein, 13 aa, Homo sapiens
ARHSIGWVAELDY

SEQ ID NO: 303 - protein, 6 aa, Homo sapiens
QSIATS

SEQ ID NO: 304 - protein, 3 aa, Homo sapiens
YAS

SEQ ID NO: 305 - protein, 9 aa, Homo sapiens
QQGAYYPLT

SEQ ID NO: 306 - protein, 120 aa, Homo sapiens; identical to SEQ ID NO: 276

SEQ ID NO: 307 - protein, 330 aa, Homo sapiens [sequence omitted]

SEQ ID NO: 308 - protein, 107 aa, Homo sapiens
DIQMTQSPSSLSASVGDRVTITCRASQSIATSLNWYQQKPGKAPKLLIYYASSLQSGVPSRFSGSGSGTDFTLTISSLQPEDFATYYCQQGAYYPLTFGQGTKVEIK

SEQ ID NO: 309 - protein, 107 aa, Homo sapiens [sequence omitted]

SEQ ID NO: 310 - protein, 8 aa, Homo sapiens
GGTFSSYA

SEQ ID NO: 311 - protein, 8 aa, Homo sapiens
IIPIFGTA

SEQ ID NO: 312 - protein, 13 aa, Homo sapiens
ARSSYGWSYEFDY

SEQ ID NO: 313 - protein, 6 aa, Homo sapiens
QSIRTY

SEQ ID NO: 314 - protein, 3 aa, Homo sapiens
AAS

SEQ ID NO: 315 - protein, 9 aa, Homo sapiens
QQAYSNPIT

SEQ ID NO: 316 - protein, 120 aa, Homo sapiens; identical to SEQ ID NO: 226

SEQ ID NO: 317 - protein, 330 aa, Homo sapiens [sequence omitted]

SEQ ID NO: 318 - protein, 107 aa, Homo sapiens
DIQMTQSPSSLSASVGDRVTITCRASQSIRTYLNWYQQKPGKAPKLLIYAASSLQSGVPSRFSGSGSGTDFTLTISSLQPEDFATYYCQQAYSNPITFGQGTKVEIK

SEQ ID NO: 319 - protein, 107 aa, Homo sapiens [sequence omitted]

SEQ ID NO: 320 - protein, 8 aa, Homo sapiens
GGTFSSYA

SEQ ID NO: 321 - protein, 8 aa, Homo sapiens
IIPIFGTA

SEQ ID NO: 322 - protein, 13 aa, Homo sapiens
ARSSYGWSYEFDY

SEQ ID NO: 323 - protein, 6 aa, Homo sapiens
QSINTN

SEQ ID NO: 324 - protein, 3 aa, Homo sapiens
AAS

SEQ ID NO: 325 - protein, 9 aa, Homo sapiens
QQARDTPIT

SEQ ID NO: 326 - protein, 120 aa, Homo sapiens; identical to SEQ ID NO: 226

SEQ ID NO: 327 - protein, 330 aa, Homo sapiens [sequence omitted]

SEQ ID NO: 328 - protein, 107 aa, Homo sapiens
DIQMTQSPSSLSASVGDRVTITCRASQSINTNLNWYQQKPGKAPKLLIYAASSLQSGVPSRFSGSGSGTDFTLTISSLQPEDFATYYCQQARDTPITFGQGTKVEIK

SEQ ID NO: 329 - protein, 107 aa, Homo sapiens [sequence omitted]

SEQ ID NO: 330 - protein, 8 aa, Homo sapiens
GYTFPSHT

SEQ ID NO: 331 - protein, 8 aa, Homo sapiens
IYPFIDST

SEQ ID NO: 332 - protein, 11 aa, Homo sapiens
ARGIRGYTMDY

SEQ ID NO: 333 - protein, 10 aa, Homo sapiens
ESVDNYGLSF

SEQ ID NO: 334 - protein, 3 aa, Homo sapiens
GAS

SEQ ID NO: 335 - protein, 9 aa, Homo sapiens
QQSKEVPYT

SEQ ID NO: 336 - protein, 118 aa, Homo sapiens
QVQLVQSGAEVKKPGASVKVSCKASGYTFPSHTIHWVRQAPGQRLEWMGSIYPFIDSTIYNQKFKDRVTITRDTSASTAYMELSSLRSEDTAVYYCARGIRGYTMDYWGQGTLVTVSS

SEQ ID NO: 337 - protein, 330 aa, Homo sapiens [sequence omitted]

SEQ ID NO: 338 - protein, 111 aa, Homo sapiens
DVVMTQSPLSLPVTLGQPASISCRASESVDNYGLSFMNWFQQRPGQSPRRLIYGASNQGSGVPDRFSGSGSGTDFTLKISRVEAEDVGVYYCQQSKEVPYTFGQGTKLEIK

SEQ ID NO: 339 - protein, 107 aa, Homo sapiens [sequence omitted]

SEQ ID NO: 340 - protein, 8 aa, Homo sapiens
GYTFTNYG

SEQ ID NO: 341 - protein, 8 aa, Homo sapiens
INPYTGEP

SEQ ID NO: 342 - protein, 12 aa, Homo sapiens
AREGYGNYIFPY

SEQ ID NO: 343 - protein, 10 aa, Homo sapiens
ESVDTYANSL

SEQ ID NO: 344 - protein, 3 aa, Homo sapiens
RAS

SEQ ID NO: 345 - protein, 9 aa, Homo sapiens
QQTNEDPRT

SEQ ID NO: 346 - protein, 119 aa, Homo sapiens
QVQLVQSGSELKKPGASVKVSCKASGYTFTNYGLNWVRQAPGQGLEWMGWINPYTGEPTYADDFKGRFVFSLDTSVSTAYLQICSLKAEDTAVYYCAREGYGNYIFPYWGQGTLVTVSS

SEQ ID NO: 347 - protein, 330 aa, Homo sapiens [sequence omitted]

SEQ ID NO: 348 - protein, 111 aa, Homo sapiens
DIVMTQTPLSLSVTPGQPASISCRASESVDTYANSLMHWYLQKPGQPPQLLIYRASNLESGVPDRFSGSGSGTDFTLKISRVEAEDVGVYYCQQTNEDPRTFGQGTKLEIK

SEQ ID NO: 349 - protein, 107 aa, Homo sapiens [sequence omitted]

SEQ ID NO: 350 - protein, 8 aa, Homo sapiens
GFTFSSYT

SEQ ID NO: 351 - protein, 8 aa, Homo sapiens
ISNSGSYT

SEQ ID NO: 352 - protein, 11 aa, Homo sapiens
ARDTVLSPFDY

SEQ ID NO: 353 - protein, 5 aa, Homo sapiens
SSISY

SEQ ID NO: 354 - protein, 3 aa, Homo sapiens
DTS

SEQ ID NO: 355 - protein, 7 aa, Homo sapiens
HQRSSFT

SEQ ID NO: 356 - protein, 118 aa, Homo sapiens
EVQLVESGGGLVQPGGSLRLSCAASGFTFSSYTMSWVRQAPGKGLEWVATISNSGSYTYYLDSVKGRFTISRDNAKNSLYLQMNSLRAEDTAVYYCARDTVLSPFDYWGQGTTVTVSS

SEQ ID NO: 357 - protein, 330 aa, Homo sapiens [sequence omitted]

SEQ ID NO: 358 - protein, 104 aa, Homo sapiens
EIVLTQSPDFQSVTPKEKVTITCSATSSISYMHWYQQKPDQSPKLLIKDTSELASGVPSRFSGSGSGTDFTLTINSLEAEDAATYYCHQRSSFTFGQGTKLEIK

SEQ ID NO: 359 - protein, 107 aa, Homo sapiens [sequence omitted]

SEQ ID NO: 360 - protein, 8 aa, Homo sapiens
GGTFSNYW

SEQ ID NO: 361 - protein, 8 aa, Homo sapiens
IYPGGGFT

SEQ ID NO: 362 - protein, 14 aa, Homo sapiens
ARYYRSDEDYSMDF

SEQ ID NO: 363 - protein, 11 aa, Homo sapiens
QSLLYSNGKTY

SEQ ID NO: 364 - protein, 3 aa, Homo sapiens
LVS

SEQ ID NO: 365 - protein, 9 aa, Homo sapiens
VQATHFPQT

SEQ ID NO: 366 - protein, 121 aa, Homo sapiens
QVQLVQSGAEVKKPGSSVKVSCKASGGTFSNYWIGWVRQAPGQGLEWMGEIYPGGGFTHYNEKFKGRVTITADESTSTAYMELSSLRSEDTAVYYCARYYRSDEDYSMDFWGQGTLVTVSS

SEQ ID NO: 367 - protein, 330 aa, Homo sapiens [sequence omitted]

SEQ ID NO: 368 - protein, 112 aa, Homo sapiens
DVVMTQSPLSLPVTLGQPASISCKSSQSLLYSNGKTYLNWFQQRPGQSPRRLIYLVSKLDSGVPDRFSGSGSGTDFTLKISRVEAEDVGVYYCVQATHFPQTFGQGTKLEIK

SEQ ID NO: 369 - protein, 107 aa, Homo sapiens [sequence omitted]

SEQ ID NO: 370 - protein, 8 aa, Homo sapiens
GYTFTHYT

SEQ ID NO: 371 - protein, 8 aa, Homo sapiens
IIPSSGYS

SEQ ID NO: 372 - protein, 16 aa, Homo sapiens
ARGAYDDYYDYYAMDY

SEQ ID NO: 373 - protein, 11 aa, Homo sapiens
QTIVHSNGNTY

SEQ ID NO: 374 - protein, 3 aa, Homo sapiens
KVS

SEQ ID NO: 375 - protein, 9 aa, Homo sapiens
FQASHVPWT

SEQ ID NO: 376 - protein, 123 aa, Homo sapiens
QVQLVQSGAEVKKPGASVKVSCKASGYTFTHYTIHWVRQAPGQGLEWMGYIIPSSGYSEYNQKFKDRVTMTRDTSTSTVYMELSSLRSEDTAVYYCARGAYDDYYDYYAMDYWGQGTLVTVSS

SEQ ID NO: 377 - protein, 330 aa, Homo sapiens [sequence omitted]

SEQ ID NO: 378 - protein, 112 aa, Homo sapiens
DIVMTQSPLSLPVTPGEPASISCRSSQTIVHSNGNTYLEWYLQKPGQSPQLLIYKVSNRFSGVPDRFSGSGSGTDFTLKISRVEAEDVGVYYCFQASHVPWTFGQGTKLEIK

SEQ ID NO: 379 - protein, 107 aa, Homo sapiens [sequence omitted]

SEQ ID NO: 380 - protein, 8 aa, Homo sapiens
GYNIKDTY

SEQ ID NO: 381 - protein, 8 aa, Homo sapiens
IDPTHGYV

SEQ ID NO: 382 - protein, 13 aa, Homo sapiens
ARDRFDPYWFLDV

SEQ ID NO: 383 - protein, 10 aa, Homo sapiens
ESVEYYGTSL

SEQ ID NO: 384 - protein, 3 aa, Homo sapiens
DAF

SEQ ID NO: 385 - protein, 9 aa, Homo sapiens
QQSRKVPWT

SEQ ID NO: 386 - protein, 120 aa, Homo sapiens
QVQLVQSGAEVKKPGASVKVSCKASGYNIKDTYMHWVRQAPGQGLEWMGRIDPTHGYVIYDPKFQGRVTMTRDTSTSTVYMELSSLRSEDTAVYYCARDRFDPYWFLDVWGQGTLVTVSS

SEQ ID NO: 387 - protein, 330 aa, Homo sapiens [sequence omitted]

SEQ ID NO: 388 - protein, 111 aa, Homo sapiens
DIVMTQTPLSLSVTPGQPASISCRASESVEYYGTSLMQWYLQKPGQSPQLLIYDAFNVESGVPDRFSGSGSGTDFTLKISRVEAEDVGVYYCQQSRKVPWTFGQGTKLEIK

SEQ ID NO: 389 - protein, 107 aa, Homo sapiens [sequence omitted]

SEQ ID NO: 390 - protein, 9 aa, Homo sapiens
GYSIASDYV

SEQ ID NO: 391 - protein, 7 aa, Homo sapiens
ISYSGST

SEQ ID NO: 392 - protein, 17 aa, Homo sapiens
ARITTVVPTGSYYGVDF

SEQ ID NO: 393 - protein, 5 aa, Homo sapiens
SSVNF

SEQ ID NO: 394 - protein, 3 aa, Homo sapiens
DTS

SEQ ID NO: 395 - protein, 9 aa, Homo sapiens
QQWSNYPFT

SEQ ID NO: 396 - protein, 124 aa, Homo sapiens
QVQLQESGPGLVKPSETLSLTCAVSGYSIASDYVWNWIRQPPGKGLEWIGYISYSGSTSNNPSLNSRVTISVDTSKNQFSLKLSSVTAADTAVYYCARITTVVPTGSYYGVDFWGQGTTVTVSS

SEQ ID NO: 397 - protein, 330 aa, Homo sapiens [sequence omitted]

SEQ ID NO: 398 - protein, 106 aa, Homo sapiens
EIVLTQSPDFQSVTPKEKVTITCSGSSSVNFMYWYQQKPDQSPKLLIKDTSNLASGVPSRFSGSGSGTDFTLTINSLEAEDAATYYCQQWSNYPFTFGQGTKLEIK

SEQ ID NO: 399 - protein, 107 aa, Homo sapiens [sequence omitted]

SEQ ID NO: 400 - protein, 8 aa, Homo sapiens
GYTFTSYW

SEQ ID NO: 401 - protein, 8 aa, Homo sapiens
IIPNTLHT

SEQ ID NO: 402 - protein, 13 aa, Homo sapiens
ARLDGDYDYALDY

SEQ ID NO: 403 - protein, 10 aa, Homo sapiens
ESVEYYGTSF

SEQ ID NO: 404 - protein, 3 aa, Homo sapiens
TAS

SEQ ID NO: 405 - protein, 9 aa, Homo sapiens
QQSRKVPYT

SEQ ID NO: 406 - protein, 120 aa, Homo sapiens
QVQLVQSGAEVKKPGASVKVSCKASGYTFTSYWLHWVRQAPGQGLEWMGYIIPNTLHTDYNQKFKDRVTMTRDTSTSTVYMELSSLRSEDTAVYYCARLDGDYDYALDYWGQGTLVTVSS

SEQ ID NO: 407 - protein, 330 aa, Homo sapiens [sequence omitted]

SEQ ID NO: 408 - protein, 111 aa, Homo sapiens
DIVMTQTPLSLSVTPGQPASISCRASESVEYYGTSFMQWYLQKPGQPPQLLIYTASNVESGVPDRFSGSGSGTDFTLKISRVEAEDVGVYYCQQSRKVPYTFGQGTKLEIK

SEQ ID NO: 409 - protein, 107 aa, Homo sapiens [sequence omitted]

SEQ ID NO: 410 - protein, 8 aa, Homo sapiens
GFTFSTYA

SEQ ID NO: 411 - protein, 8 aa, Homo sapiens
ISSGGSDT

SEQ ID NO: 412 - protein, 14 aa, Homo sapiens
ARPTYYGIFSYFDY

SEQ ID NO: 413 - protein, 5 aa, Homo sapiens
SSVSY

SEQ ID NO: 414 - protein, 3 aa, Homo sapiens
STS

SEQ ID NO: 415 - protein, 8 aa, Homo sapiens
HQWRTYPT

SEQ ID NO: 416 - protein, 121 aa, Homo sapiens
EVQLVESGGGLVQPGGSLRLSCAASGFTFSTYAMSWVRQAPGKGLEWVATISSGGSDTYYPDTVKGRFTISRDNAKNSLYLQMNSLRAEDTAVYYCARPTYYGIFSYFDYWGQGTLVTVSS

SEQ ID NO: 417 - protein, 330 aa, Homo sapiens [sequence omitted]

SEQ ID NO: 418 - protein, 105 aa, Homo sapiens
DIQLTQSPSFLSASVGDRVTITCSASSSVSYMHWYQQKPGKAPKLLIYSTSNLASGVPSRFSGSGSGTEFTLTISSLQPEDFATYYCHQWRTYPTFGQGTKLEIK

SEQ ID NO: 419 - protein, 107 aa, Homo sapiens [sequence omitted]

SEQ ID NO: 420 - protein, 8 aa, Homo sapiens
GFSLTNYG

SEQ ID NO: 421 - protein, 7 aa, Homo sapiens
IWRGGNT

SEQ ID NO: 422 - protein, 11 aa, Homo sapiens
ARSMVSYTVDY

SEQ ID NO: 423 - protein, 5 aa, Homo sapiens
SSVSY

SEQ ID NO: 424 - protein, 3 aa, Homo sapiens
DTS

SEQ ID NO: 425 - protein, 9 aa, Homo sapiens
QQWSSYPPT

SEQ ID NO: 426 - protein, 117 aa, Homo sapiens
QVQLQESGPGLVKPSETLSLTCTVSGFSLTNYGVHWIRQPPGKGLEWIGVIWRGGNTDYNAAFMSRVTISVDTSKNQFSLKLSSVTAADTAVYYCARSMVSYTVDYWGQGTLVTVSS

SEQ ID NO: 427 - protein, 330 aa, Homo sapiens [sequence omitted]

SEQ ID NO: 428 - protein, 106 aa, Homo sapiens
EIVLTQSPDFQSVTPKEKVTITCSASSSVSYMHWYQQKPDQSPKLLIKDTSNLASGVPSRFSGSGSGTDFTLTINSLEAEDAATYYCQQWSSYPPTFGQGTKLEIK

SEQ ID NO: 429 - protein, 107 aa, Homo sapiens [sequence omitted]

SEQ ID NO: 430 - protein, 8 aa, Homo sapiens
GYTFTDYW

SEQ ID NO: 431 - protein, 8 aa, Homo sapiens
IETSLNYP

SEQ ID NO: 432 - protein, 13 aa, Homo sapiens
ARWGIYGNPWFAY

SEQ ID NO: 433 - protein, 10 aa, Homo sapiens
ESVDSYVNSF

SEQ ID NO: 434 - protein, 3 aa, Homo sapiens
RAS

SEQ ID NO: 435 - protein, 9 aa, Homo sapiens
QQSNEDPYT

SEQ ID NO: 436 - protein, 120 aa, Homo sapiens
QVQLVQSGAEVKKPGASVKVSCKASGYTFTDYWMHWVRQAPGQGLEWMGAIETSLNYPSYNQKFKGRVTMTRDTSTSTVYMELSSLRSEDTAVYYCARWGIYGNPWFAYWGRGTLVTVSS

SEQ ID NO: 437 - protein, 330 aa, Homo sapiens [sequence omitted]

SEQ ID NO: 438 - protein, 111 aa, Homo sapiens
DIVMTQTPLSLSVTPGQPASISCRASESVDSYVNSFVHWYLQKPGQPPQLLIYRASNLESGVPDRFSGSGSGTDFTLKISRVEAEDVGVYYCQQSNEDPYTFGQGTKLEIK

SEQ ID NO: 439 - protein, 107 aa, Homo sapiens [sequence omitted]

SEQ ID NO: 440 - protein, 8 aa, Homo sapiens
GYTFTNYG

SEQ ID NO: 441 - protein, 8 aa, Homo sapiens
INTYTGES

SEQ ID NO: 442 - protein, 12 aa, Homo sapiens
ARDYYGIYVSAY

SEQ ID NO: 443 - protein, 10 aa, Homo sapiens
ESVDNYANSF

SEQ ID NO: 444 - protein, 3 aa, Homo sapiens
RAS

SEQ ID NO: 445 - protein, 9 aa, Homo sapiens
QQSHEDPYT

SEQ ID NO: 446 - protein, 119 aa, Homo sapiens
QVQLVQSGSELKKPGASVKVSCKASGYTFTNYGMTWVRQAPGQGLEWMGWINTYTGESTYADDFKGRFVFSLDTSVSTAYLQICSLKAEDTAVYYCARDYYGIYVSAYWGQGTLVTVSS

SEQ ID NO: 447 - protein, 330 aa, Homo sapiens [sequence omitted]

SEQ ID NO: 448 - protein, 111 aa, Homo sapiens
DIVMTQTPLSLSVTPGQPASISCRASESVDNYANSFMHWYLQKPGQSPQLLIYRASNLESGVPDRFSGSGSGTDFTLKISRVEAEDVGVYYCQQSHEDPYTFGQGTKLEIK

SEQ ID NO: 449 - protein, 107 aa, Homo sapiens [sequence omitted]

SEQ ID NO: 450 - protein, 8 aa, Homo sapiens
GYTFTSHW

SEQ ID NO: 451 - protein, 8 aa, Homo sapiens
INPRDGRT

SEQ ID NO: 452 - protein, 16 aa, Homo sapiens
ARGDFHYGDYFWYFDV

SEQ ID NO: 453 - protein, 6 aa, Homo sapiens
QNVHGA

SEQ ID NO: 454 - protein, 3 aa, Homo sapiens
MAS

SEQ ID NO: 455 - protein, 8 aa, Homo sapiens
LQHWNYLT

SEQ ID NO: 456 - protein, 123 aa, Homo sapiens
QVQLVQSGAEVKKPGASVKVSCKASGYTFTSHWMHWVRQAPGQGLEWMGEINPRDGRTNYNEKFKTRVTMTRDTSTSTVYMELSSLRSEDTAVYYCARGDFHYGDYFWYFDVWGQGTLVTVSS

SEQ ID NO: 457 - protein, 330 aa, Homo sapiens [sequence omitted]

SEQ ID NO: 458 - protein, 106 aa, Homo sapiens
DIQMTQSPSSLSASVGDRVTITCKASQNVHGAVAWYQQKPGKVPKLLIYMASNRPTGVPSRFSGSGSGTDFTLTISSLQPEDVATYYCLQHWNYLTFGQGTKLEIK

SEQ ID NO: 459 - protein, 107 aa, Homo sapiens [sequence omitted]

SEQ ID NO: 460 - protein, 8 aa, Homo sapiens
GYTFTHYW

SEQ ID NO: 461 - protein, 8 aa, Homo sapiens
IYPGDGDT

SEQ ID NO: 462 - protein, 10 aa, Homo sapiens
ARRDYDYGDY

SEQ ID NO: 463 - protein, 5 aa, Homo sapiens
SSVSH

SEQ ID NO: 464 - protein, 3 aa, Homo sapiens
LTS

SEQ ID NO: 465 - protein, 9 aa, Homo sapiens
QQYQTYPPT

SEQ ID NO: 466 - protein, 117 aa, Homo sapiens
QVQLVQSGAEVKKPGASVKVSCKASGYTFTHYWIQWVRQAPGQGLEWMGAIYPGDGDTRYTQKFKGRVTMTRDTSTSTVYMELSSLRSEDTAVYYCARRDYDYGDYWGQGTTVTVSS

SEQ ID NO: 467 - protein, 330 aa, Homo sapiens [sequence omitted]

SEQ ID NO: 468 - protein, 106 aa, Homo sapiens
EIVLTQSPDFQSVTPKEKVTITCSASSSVSHMYWYQQKPDQSPKLLIKLTSNLASGVPSRFSGSGSGTDFTLTINSLEAEDAATYYCQQYQTYPPTFGQGTRLEIK

SEQ ID NO: 469 - protein, 107 aa, Homo sapiens [sequence omitted]

SEQ ID NO: 470 - protein, 9 aa, Homo sapiens
GFSITSDFA

SEQ ID NO: 471 - protein, 7 aa, Homo sapiens
ITYSGFT

SEQ ID NO: 472 - protein, 14 aa, Homo sapiens
ARQEYGNYVWYFDV

SEQ ID NO: 473 - protein, 10 aa, Homo sapiens
ESVEYYGTSL

SEQ ID NO: 474 - protein, 3 aa, Homo sapiens
AAS

SEQ ID NO: 475 - protein, 9 aa, Homo sapiens
QQSRKVPWT

SEQ ID NO: 476 - protein, 121 aa, Homo sapiens
QVQLQESGPGLVKPSETLSLTCAVSGFSITSDFAWNWIRQPPGKGLEWIGYITYSGFTNYNPSLESRVTISVDTSKNQFSLKLSSVTAADTAVYYCARQEYGNYVWYFDVWGQGTLVTVSS

SEQ ID NO: 477 - protein, 330 aa, Homo sapiens [sequence omitted]

SEQ ID NO: 478 - protein, 111 aa, Homo sapiens
DIVMTQTPLSLSVTPGQPASISCRASESVEYYGTSLLQWYLQKPGQPPQLLIYAASNVESGVPDRFSGSGSGTDFTLKISRVEAEDVGVYYCQQSRKVPWTFGQGTKLEIK

SEQ ID NO: 479 - protein, 107 aa, Homo sapiens [sequence omitted]

SEQ ID NO: 480 - protein, 9 aa, Homo sapiens
GYSITSGYF

SEQ ID NO: 481 - protein, 7 aa, Homo sapiens
MGYDGRI

SEQ ID NO: 482 - protein, 14 aa, Homo sapiens
AREGDYYGSGFAYW

SEQ ID NO: 483 - protein, 6 aa, Homo sapiens
QNVNTN

SEQ ID NO: 484 - protein, 3 aa, Homo sapiens
SAS

SEQ ID NO: 485 - protein, 120 aa, Homo sapiens
QVQLQESGPGLVKPSETLSLTCAVSGYSITSGYFWNWIRQPPGKGLEWIGYMGYDGRIFYSPSLKNRVTISVDTSKNQFSLKLSSVTAADTAVYYCAREGDYYGSGFAYWGQGTLVTVSS

SEQ ID NO: 486 - protein, 120 aa, Homo sapiens; identical to SEQ ID NO: 485

SEQ ID NO: 487 - protein, 330 aa, Homo sapiens [sequence omitted]

SEQ ID NO: 488 - protein, 107 aa, Homo sapiens
DIQMTQSPSSLSASVGDRVTITCKASQNVNTNLAWYQQKPGKVPKLLIYSASYRYSGVPSRFSGSGSGTDFTLTISSLQPEDVATYYCQQYNNYPLTFGQGTKLEIK

SEQ ID NO: 489 - protein, 107 aa, Homo sapiens [sequence omitted]

SEQ ID NO: 490 - protein, 8 aa, Homo sapiens
GYTFTSYW

SEQ ID NO: 491 - protein, 8 aa, Homo sapiens
INPSNGLT

SEQ ID NO: 492 - protein, 15 aa, Homo sapiens
ARSYDYDGDYYAMDY

SEQ ID NO: 493 - protein, 11 aa, Homo sapiens
QSIVHSNGNTY

SEQ ID NO: 494 - protein, 3 aa, Homo sapiens
KVS

SEQ ID NO: 495 - protein, 9 aa, Homo sapiens
FQASHVPWT

SEQ ID NO: 496 - protein, 122 aa, Homo sapiens
QVQLVQSGAEVKKPGASVKVSCKASGYTFTSYWIHWVRQAPGQGLEWMGEINPSNGLTNYNEKFRNRVTMTRDTSTSTVYMELSSLRSEDTAVYYCARSYDYDGDYYAMDYWGQGTLVTVSS

SEQ ID NO: 497 - protein, 330 aa, Homo sapiens [sequence omitted]

SEQ ID NO: 498 - protein, 112 aa, Homo sapiens
DIVMTQSPLSLPVTPGEPASISCRSTQSIVHSNGNTYLEWYLQKPGQSPQLLIYKVSNRFSGVPDRFSGSGSGTDFTLKISRVEAEDVGVYYCFQASHVPWTFGQGTKLEIK

SEQ ID NO: 499 - protein, 107 aa, Homo sapiens [sequence omitted]

SEQ ID NO: 500 - protein, 8 aa, Homo sapiens
GYPFTGYF

SEQ ID NO: 501 - protein, 8 aa, Homo sapiens
INPYNGGT

SEQ ID NO: 502 - protein, 12 aa, Homo sapiens
ARWTFDGLFMDY

SEQ ID NO: 503 - protein, 10 aa, Homo sapiens
ENVDKYGISF

SEQ ID NO: 504 - protein, 3 aa, Homo sapiens
ATS

SEQ ID NO: 505 - protein, 9 aa, Homo sapiens
QQSKEDPYT

SEQ ID NO: 506 - protein, 119 aa, Homo sapiens
QVQLVQSGAEVKKPGASVKVSCKASGYPFTGYFMHWVRQAPGQGLEWMGRINPYNGGTVYNQNFNDRVTMTRDTSTSTVYMELSSLRSEDTAVYYCARWTFDGLFMDYWGQGTTVTVSS

SEQ ID NO: 507 - protein, 330 aa, Homo sapiens [sequence omitted]

SEQ ID NO: 508 - protein, 111 aa, Homo sapiens
DVVMTQSPLSLPVTLGQPASISCRASENVDKYGISFMNWFQQRPGQSPRRLIYATSNQGSGVPDRFSGSGSGTDFTLKISRVEAEDVGVYYCQQSKEDPYTFGQGTKLEIK

SEQ ID NO: 509 - protein, 107 aa, Homo sapiens [sequence omitted]

SEQ ID NO: 510 - protein, 8 aa, Homo sapiens
DYIFSSYW

SEQ ID NO: 511 - protein, 8 aa, Homo sapiens
IFPGSGGT

SEQ ID NO: 512 - protein, 15 aa, Homo sapiens
ARAIYYDYDMYYFDS

SEQ ID NO: 513 - protein, 6 aa, Homo sapiens
QDISSY

SEQ ID NO: 514 - protein, 3 aa, Homo sapiens
YTS

SEQ ID NO: 515 - protein, 9 aa, Homo sapiens
QHVNTLPWT

SEQ ID NO: 516 - protein, 122 aa, Homo sapiens
QVQLVQSGAEVKKPGASVKVSCKASDYIFSSYWIQWVRQAPGQGLEWMGEIFPGSGGTNYNEKFKGRVTMTRDTSTSTVYMELSSLRSEDTAVYYCARAIYYDYDMYYFDSWGQGTTVTVSS

SEQ ID NO: 517 - protein, 330 aa, Homo sapiens [sequence omitted]

SEQ ID NO: 518 - protein, 107 aa, Homo sapiens
DIQMTQSPSSLSASVGDRVTITCRASQDISSYLNWYQQKPGKVPKLLIYYTSRLHSGVPSRFSGSGSGTDFTLTISSLQPEDVATYYCQHVNTLPWTFGQGTKLEIK

SEQ ID NO: 519 - protein, 107 aa, Homo sapiens [sequence omitted]

SEQ ID NO: 520 - protein, 8 aa, Homo sapiens
GYTFTDYT

SEQ ID NO: 521 - protein, 8 aa, Homo sapiens
INPYNGGT

SEQ ID NO: 522 - protein, 13 aa, Homo sapiens
ARHYGNYNWYFDV

SEQ ID NO: 523 - protein, 6 aa, Homo sapiens
QYVNTA

SEQ ID NO: 524 - protein, 3 aa, Homo sapiens
SAS

SEQ ID NO: 525 - protein, 9 aa, Homo sapiens
QQHFTTPIT

SEQ ID NO: 526 - protein, 120 aa, Homo sapiens
QVQLVQSGAEVKKPGASVKVSCKASGYTFTDYTMNWVRQAPGQRLEWMGLINPYNGGTTYNQKFKGRVTITRDTSASTAYMELSSLRSEDTAVYYCARHYGNYNWYFDVWGQGTTVTVSS

SEQ ID NO: 527 - protein, 330 aa, Homo sapiens [sequence omitted]

SEQ ID NO: 528 - protein, 107 aa, Homo sapiens
DIQLTQSPSFLSASVGDRVTITCKASQYVNTAVAWYQQKPGKAPKLLIYSASYRYTGVPSRFSGSGSGTEFTLTISSLQPEDFATYYCQQHFTTPITFGQGTKLEIK

SEQ ID NO: 529 - protein, 107 aa, Homo sapiens [sequence omitted]

SEQ ID NO: 530 - protein, 8 aa, Homo sapiens
GYTFTNYG

SEQ ID NO: 531 - protein, 8 aa, Homo sapiens
INTYTGEP

SEQ ID NO: 532 - protein, 10 aa, Homo sapiens
ARNYGNYVAY

SEQ ID NO: 533 - protein, 6 aa, Homo sapiens
QDINSY

SEQ ID NO: 534 - protein, 3 aa, Homo sapiens
RAN

SEQ ID NO: 535 - protein, 9 aa, Homo sapiens
LQYDEFPLT

SEQ ID NO: 536 - protein, 117 aa, Homo sapiens
QVQLVQSGSELKKPGASVKVSCKASGYTFTNYGMNWVRQAPGQGLEWMGWINTYTGEPTYADDFKGRFVFSLDTSVSTAYLQICSLKAEDTAVYYCARNYGNYVAYWGQGTTVTVSS

SEQ ID NO: 537 - protein, 330 aa, Homo sapiens [sequence omitted]

SEQ ID NO: 538 - protein, 107 aa, Homo sapiens
DIQMTQSPSSLSASVGDRVTITCKASQDINSYLSWFQQKPGKAPKSLIYRANRLVDGVPSRFSGSGSGTDFTLTISSLQPEDFATYYCLQYDEFPLTFGQGTRLEIK

SEQ ID NO: 539 - protein, 107 aa, Homo sapiens [sequence omitted]

SEQ ID NO: 540 - protein, 8 aa, Homo sapiens
GYTFSNYW

SEQ ID NO: 541 - protein, 8 aa, Homo sapiens
ILPGSGNV

SEQ ID NO: 542 - protein, 16 aa, Homo sapiens
ATPPHYYGYDYYDVNY

SEQ ID NO: 543 - protein, 11 aa, Homo sapiens
QSLLDSDGKTY

SEQ ID NO: 544 - protein, 3 aa, Homo sapiens
LVS

SEQ ID NO: 545 - protein, 9 aa, Homo sapiens
WQGTHFPQT

SEQ ID NO: 546 - protein, 123 aa, Homo sapiens
EVQLVQSGAEVKKPGATVKISCKVSGYTFSNYWIEWVQQAPGKGLEWMGEILPGSGNVHYNEKFKGRVTITADTSTDTAYMELSSLRSEDTAVYYCATPPHYYGYDYYDVNYWGQGTLVTVSS

SEQ ID NO: 547 - protein, 330 aa, Homo sapiens [sequence omitted]

SEQ ID NO: 548 - protein, 112 aa, Homo sapiens
DVVMTQSPLSLPVTLGQPASISCKSSQSLLDSDGKTYLNWFQQRPGQSPRRLIYLVSELDSGVPDRFSGSGSGTDFTLKISRVEAEDVGVYYCWQGTHFPQTFGQGTKLEIK

SEQ ID NO: 549 - protein, 107 aa, Homo sapiens [sequence omitted]

SEQ ID NO: 550 - protein, 8 aa, Homo sapiens
GYVFSRSW

SEQ ID NO: 551 - protein, 8 aa, Homo sapiens
IYPGDGDT

SEQ ID NO: 552 - protein, 6 aa, Homo sapiens
AARWFL

SEQ ID NO: 553 - protein, 12 aa, Homo sapiens
QSLLNSGDQKSY

SEQ ID NO: 554 - protein, 3 aa, Homo sapiens
GAS

SEQ ID NO: 555 - protein, 9 aa, Homo sapiens
QNDHSYPLT

SEQ ID NO: 556 - protein, 113 aa, Homo sapiens
QVQLVQSGAEVKKPGSSVKVSCKASGYVFSRSWINWVRQAPGQGLEWMGRIYPGDGDTNYNGKFKGRVTITADESTSTAYMELSSLRSEDTAVYYCARRWFLWGQGTLVTVSS

SEQ ID NO: 557 - protein, 330 aa, Homo sapiens [sequence omitted]

SEQ ID NO: 558 - protein, 113 aa, Homo sapiens
DIVMTQSPDSLAVSLGERATINCKSSQSLLNSGDQKSYLAWYQQKPGQPPKLLIYGASTRESGVPDRFSGSGSGTDFTLTISSLQAEDVAVYYCQNDHSYPLTFGQGTKLEIK

SEQ ID NO: 559 - protein, 107 aa, Homo sapiens [sequence omitted]

SEQ ID NO: 560 - protein, 8 aa, Homo sapiens
GFTFSSYA

SEQ ID NO: 561 - protein, 8 aa, Homo sapiens
ISSGGSHT

SEQ ID NO: 562 - protein, 11 aa, Homo sapiens
ARRGNLYDGPY

SEQ ID NO: 563 - protein, 5 aa, Homo sapiens
SSVSH

SEQ ID NO: 564 - protein, 3 aa, Homo sapiens
DTS

SEQ ID NO: 565 - protein, 9 aa, Homo sapiens
QQWNFYPFT

SEQ ID NO: 566 - protein, 118 aa, Homo sapiens
EVQLVESGGGLVQPGGSLRLSCAASGFTFSSYAMSWVRQAPGKGLEWVAEISSGGSHTYYPDTVTGRFTISRDNAKNSLYLQMNSLRAEDTAVYYCARRGNLYDGPYWGQGTLVTVSS

SEQ ID NO: 567 - protein, 330 aa, Homo sapiens [sequence omitted]

SEQ ID NO: 568 - protein, 106 aa, Homo sapiens
EIVLTQSPATLSLSPGERATLSCSASSSVSHMYWYQQKPGQAPRLLIYDTSKLASGIPARFSGSGSGTDFTLTISSLEPEDFAVYYCQQWNFYPFTFGQGTKLEIK

SEQ ID NO: 569 - protein, 107 aa, Homo sapiens [sequence omitted]

SEQ ID NO: 570 - protein, 8 aa, Homo sapiens
GYTFTDYV

SEQ ID NO: 571 - protein, 8 aa, Homo sapiens
IYPGSGNT

SEQ ID NO: 572 - protein, 10 aa, Homo sapiens
ARVLVSVMDY

SEQ ID NO: 573 - protein, 5 aa, Homo sapiens
SSINY

SEQ ID NO: 574 - protein, 3 aa, Homo sapiens
DTS

SEQ ID NO: 575 - protein, 9 aa, Homo sapiens
HQRSSYPWT

SEQ ID NO: 576 - protein, 117 aa, Homo sapiens
QVQLVQSGAEVKKPGASVKVSCKASGYTFTDYVISWVRQAPGQGLEWMGEIYPGSGNTYYYEKFKGRVTMTRDTSTSTVYMELSSLRSEDTAVYYCARVLVSVMDYWGQGTLVTVSS

SEQ ID NO: 577 - protein, 330 aa, Homo sapiens [sequence omitted]

SEQ ID NO: 578 - protein, 106 aa, Homo sapiens
EIVLTQSPDFQSVTPKEKVTITCSASSSINYITWYQQKPDQSPKLLIKDTSKLASGVPSRFSGSGSGTDFTLTINSLEAEDAATYYCHQRSSYPWTFGQGTRLEIK

SEQ ID NO: 579 - protein, 107 aa, Homo sapiens [sequence omitted]

SEQ ID NO: 580 - protein, 8 aa, Homo sapiens
GYIFTDYN

SEQ ID NO: 581 - protein, 8 aa, Homo sapiens
INPKYDST

SEQ ID NO: 582 - protein, 10 aa, Homo sapiens
AADGSSAMDY

SEQ ID NO: 583 - protein, 12 aa, Homo sapiens
QSLLNSGHQKNY

SEQ ID NO: 584 - protein, 3 aa, Homo sapiens
GAS

SEQ ID NO: 585 - protein, 9 aa, Homo sapiens
QNDHRYPLT

SEQ ID NO: 586 - protein, 117 aa, Homo sapiens
QMQLVQSGPEVKKPGTSVKVSCKASGYIFTDYNIDWVRQARGQRLEWIGDINPKYDSTRYNQKFKGRVTITRDMSTSTAYMELSSLRSEDTAVYYCAADGSSAMDYWGQGTTVTVSS

SEQ ID NO: 587 - protein, 330 aa, Homo sapiens [sequence omitted]

SEQ ID NO: 588 - protein, 113 aa, Homo sapiens
DIVMTQSPDSLAVSLGERATINCKSSQSLLNSGHQKNYLAWYQQKPGQPPKLLIYGASTRESGVPDRFSGSGSGTDFTLTISSLQAEDVAVYYCQNDHRYPLTFGQGTKLEIK

SEQ ID NO: 589 - protein, 107 aa, Homo sapiens [sequence omitted]

SEQ ID NO: 590 - protein, 8 aa, Homo sapiens
GFNIKDYY

SEQ ID NO: 591 - protein, 8 aa, Homo sapiens
IDPENGNT

SEQ ID NO: 592 - protein, 10 aa, Homo sapiens
ARDYGYFDYW

SEQ ID NO: 593 - protein, 5 aa, Homo sapiens
SSVIY

SEQ ID NO: 594 - protein, 3 aa, Homo sapiens
STS

SEQ ID NO: 595 - protein, 9 aa, Homo sapiens
QQRSSYPHT

SEQ ID NO: 596 - protein, 116 aa, Homo sapiens
QVQLVQSGAEVKKPGSSVKVSCKASGFNIKDYYIHWVRQAPGQGLEWMGWIDPENGNTIYDPKFQGRVTITADESTSTAYMELSSLRSEDTAVYYCARDYGYFDYWGQGTLVTVSS

SEQ ID NO: 597 - protein, 330 aa, Homo sapiens [sequence omitted]

SEQ ID NO: 598 - protein, 106 aa, Homo sapiens
DVVMTQSPAFLSVTPGEKVTITCSAGSSVIYMYWYQQKPDQAPKLLIKSTSNLASGVPSRFSGSGSGTDFTFTISSLEAEDAATYYCQQRSSYPHTFGQGTKLEIK

SEQ ID NO: 599 - protein, 107 aa, Homo sapiens [sequence omitted]

SEQ ID NO: 600 - protein, 8 aa, Homo sapiens
GYTFTDYW

SEQ ID NO: 601 - protein, 8 aa, Homo sapiens
IYPSHSYT

SEQ ID NO: 602 - protein, 14 aa, Homo sapiens
ARGGYRYPYYAMDY

SEQ ID NO: 603 - protein, 6 aa, Homo sapiens
ENIYGA

SEQ ID NO: 604 - protein, 3 aa, Homo sapiens
GAT

SEQ ID NO: 605 - protein, 9 aa, Homo sapiens
QNVLSTPYT

SEQ ID NO: 606 - protein, 121 aa, Homo sapiens
QVQLVQSGAEVKKPGASVKVSCKASGYTFTDYWINWVRQAPGQGLEWMGNIYPSHSYTNYNQEFKDRVTMTRDTSTSTVYMELSSLRSEDTAVYYCARGGYRYPYYAMDYWGQGTTVTVSS

SEQ ID NO: 607 - protein, 330 aa, Homo sapiens [sequence omitted]

SEQ ID NO: 608 - protein, 107 aa, Homo sapiens
AIQLTQSPSSLSASVGDRVTITCGASENIYGALNWYQQKPGKAPKLLIYGATNLADGVPSRFSGSGSGTDFTLTISSLQPEDFATYYCQNVLSTPYTFGQGTKLEIK

SEQ ID NO: 609 - protein, 107 aa, Homo sapiens [sequence omitted]

SEQ ID NO: 610 - protein, 8 aa, Homo sapiens
GYTFTNYW

SEQ ID NO: 611 - protein, 8 aa, Homo sapiens
INPSSGYT

SEQ ID NO: 612 - protein, 11 aa, Homo sapiens
ARDGGSVLFGY

SEQ ID NO: 613 - protein, 12 aa, Homo sapiens
QSLLNSGIRKNY

SEQ ID NO: 614 - protein, 3 aa, Homo sapiens
SAS

SEQ ID NO: 615 - protein, 8 aa, Homo sapiens
KQSYNLYT

SEQ ID NO: 616 - protein, 118 aa, Homo sapiens
QVQLVQSGAEVKKPGASVKVSCKASGYTFTNYWMHWVRQAPGQGLEWMGYINPSSGYTEYNQKFKDRVTMTRDTSTSTVYMELSSLRSEDTAVYYCARDGGSVLFGYWGQGTTVTVSS

SEQ ID NO: 617 - protein, 330 aa, Homo sapiens [sequence omitted]

SEQ ID NO: 618 - protein, 112 aa, Homo sapiens
DIVMTQSPDSLAVSLGERATINCKSSQSLLNSGIRKNYLAWYQQKPGQPPKLLIYSASTRESGVPDRFSGSGSGTDFTLTISSLQAEDVAVYYCKQSYNLYTFGQGTKLEIK

SEQ ID NO: 619 - protein, 107 aa, Homo sapiens [sequence omitted]

SEQ ID NO: 620 - protein, 9 aa, Homo sapiens
AYSITSDYA

SEQ ID NO: 621 - protein, 7 aa, Homo sapiens
ITYSGST

SEQ ID NO: 622 - protein, 6 aa, Homo sapiens
ARSFGY

SEQ ID NO: 623 - protein, 6 aa, Homo sapiens
QSIGTS

SEQ ID NO: 624 - protein, 3 aa, Homo sapiens
YAS

SEQ ID NO: 625 - protein, 7 aa, Homo sapiens
QQINSWP

SEQ ID NO: 626 - protein, 113 aa, Homo sapiens
QVQLQESGPGLVKPSETLSLTCAVSAYSITSDYAWNWIRQPPGKGLEWIGYITYSGSTRYNPSLKSRVTISVDTSKNQFSLKLSSVTAADTAVYYCARSFGYWGQGTTVTVSS

SEQ ID NO: 627 - protein, 330 aa, Homo sapiens [sequence omitted]

SEQ ID NO: 628 - protein, 107 aa, Homo sapiens
EIVMTQSPATLSVSPGERATLSCRASQSIGTSIHWYQQKPGQAPRLLIYYASESISGIPARFSGSGSGTEFTLTISSLQSEDFAVYYCQQINSWPTTFGQGTKLEIK

SEQ ID NO: 629 - protein, 107 aa, Homo sapiens [sequence omitted]

SEQ ID NO: 630 - protein, 8 aa, Homo sapiens
GFNIKDYY

SEQ ID NO: 631 - protein, 8 aa, Homo sapiens
IDPENGDT

SEQ ID NO: 632 - protein, 10 aa, Homo sapiens
ARDMGSSYVY

SEQ ID NO: 633 - protein, 5 aa, Homo sapiens
SSVIY

SEQ ID NO: 634 - protein, 3 aa, Homo sapiens
STS

SEQ ID NO: 635 - protein, 9 aa, Homo sapiens
QQRSSYPFT

SEQ ID NO: 636 - protein, 117 aa, Homo sapiens
QVQLVQSGAEVKKPGASVKVSCKASGFNIKDYYMHWVRQAPGQGLEWMGWIDPENGDTEYAPKFQDRVTMTRDTSTSTVYMELSSLRSEDTAVYYCARDMGSSYVYWGRGTLVTVSS

SEQ ID NO: 637 - protein, 330 aa, Homo sapiens [sequence omitted]

SEQ ID NO: 638 - protein, 106 aa, Homo sapiens
DVVMTQSPAFLSVTPGEKVTITCSASSSVIYMHWYQQKPDQAPKLLIKSTSNLASGVPSRFSGSGSGTDFTFTISSLEAEDAATYYCQQRSSYPFTFGQGTKLEIK

SEQ ID NO: 639 - protein, 107 aa, Homo sapiens [sequence omitted]

SEQ ID NO: 640 - protein, 10 aa, Homo sapiens
GFSLSTSGMG

SEQ ID NO: 641 - protein, 7 aa, Homo sapiens
IYWDDDK

SEQ ID NO: 642 - protein, 13 aa, Homo sapiens
ARRPDYFGSSYVD

SEQ ID NO: 643 - protein, 10 aa, Homo sapiens
ESVDTYGNSF

SEQ ID NO: 644 - protein, 3 aa, Homo sapiens
LAS

SEQ ID NO: 645 - protein, 9 aa, Homo sapiens
QQNNEDPWT

SEQ ID NO: 646 - protein, 121 aa, Homo sapiens
QVTLKESGPALVKPTQTLTLTCTFSGFSLSTSGMGVSWIRQPPGKALEWLAHIYWDDDKRYNPSLKSRLTISKDTSKNQVVLTMTNMDPVDTATYYCARRPDYFGSSYVDWGQGTTVTVSS

SEQ ID NO: 647 - protein, 330 aa, Homo sapiens [sequence omitted]

SEQ ID NO: 648 - protein, 111 aa, Homo sapiens
DIVMTQTPLSLSVTPGQPASISCRASESVDTYGNSFMHWYLQKPGQPPQLLIYLASNLDFGVPDRFSGSGSGTDFTLKISRVEAEDVGVYYCQQNNEDPWTFGQGTKLEIK

SEQ ID NO: 649 - protein, 107 aa, Homo sapiens [sequence omitted]

SEQ ID NO: 650 - protein, 9 aa, Homo sapiens
GYSITSDYA

SEQ ID NO: 651 - protein, 7 aa, Homo sapiens
ITYSGST

SEQ ID NO: 652 - protein, 14 aa, Homo sapiens
ARSHYGSTYWYFDV

SEQ ID NO: 653 - protein, 10 aa, Homo sapiens
ESVEYYGTSL

SEQ ID NO: 654 - protein, 3 aa, Homo sapiens
AAS

SEQ ID NO: 655 - protein, 9 aa, Homo sapiens
QQTRKVPWT

SEQ ID NO: 656 - protein, 121 aa, Homo sapiens
QVQLQESGPGLVKPSETLSLTCAVSGYSITSDYAWNWIRQPPGKGLEWIGFITYSGSTNYNPSLKSRVTISVDTSKNQFSLKLSSVTAADTAVYYCARSHYGSTYWYFDVWGQGTTVTVSS

SEQ ID NO: 657 - protein, 330 aa, Homo sapiens [sequence omitted]

SEQ ID NO: 658 - protein, 111 aa, Homo sapiens
DIVMTQTPLSLSVTPGQPASISCRASESVEYYGTSLMQWYLQKPGQSPQLLIYAASNVESGVPDRFSGSGSGTDFTLKISRVEAEDVGVYYCQQTRKVPWTFGQGTRLEIK

SEQ ID NO: 659 - protein, 107 aa, Homo sapiens [sequence omitted]

SEQ ID NO: 660 - protein, 8 aa, Homo sapiens
GYTFTSYD

SEQ ID NO: 661 - protein, 8 aa, Homo sapiens
IYPGDGYT

SEQ ID NO: 662 - protein, 12 aa, Homo sapiens
ARWGYGSYAMDY

SEQ ID NO: 663 - protein, 11 aa, Homo sapiens
QSLVHSNGNTY

SEQ ID NO: 664 - protein, 3 aa, Homo sapiens
KVS

SEQ ID NO: 665 - protein, 9 aa, Homo sapiens
SQSTHVPPT

SEQ ID NO: 666 - protein, 119 aa, Homo sapiens
QVQLVQSGAEVKKPGASVKVSCKASGYTFTSYDINWVRQAPGQGLEWMGWIYPGDGYTEYNEKFRGRVTMTRDTSTSTVYMELSSLRSEDTAVYYCARWGYGSYAMDYWGQGTLVTVSS

SEQ ID NO: 667 - protein, 330 aa, Homo sapiens [sequence omitted]

SEQ ID NO: 668 - protein, 112 aa, Homo sapiens
DIVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQSTHVPPTFGQGTKLEIK

SEQ ID NO: 669 - protein, 107 aa, Homo sapiens [sequence omitted]

SEQ ID NO: 670 - protein, 8 aa, Homo sapiens
GYTFTSYW

SEQ ID NO: 671 - protein, 8 aa, Homo sapiens
INPSTGYP

SEQ ID NO: 672 - protein, 15 aa, Homo sapiens
ARSYYDYDGGAWFAY

SEQ ID NO: 673 - protein, 10 aa, Homo sapiens
ESVEYYGTSL

SEQ ID NO: 674 - protein, 3 aa, Homo sapiens
AAS

SEQ ID NO: 675 - protein, 9 aa, Homo sapiens
QQSRKVPST

SEQ ID NO: 676 - protein, 122 aa, Homo sapiens
QVQLVQSGAEVKKPGASVKVSCKASGYTFTSYWMHWVRQAPGQGLEWMGYINPSTGYPGYNQKFKDRVTMTRDTSTSTVYMELSSLRSEDTAVYYCARSYYDYDGGAWFAYWGQGTTVTVSS

SEQ ID NO: 677 - protein, 330 aa, Homo sapiens [sequence omitted]

SEQ ID NO: 678 - protein, 111 aa, Homo sapiens
DIVMTQTPLSLSVTPGQPASISCRASESVEYYGTSLMQWYLQKPGQPPQLLIYAASNVESGVPDRFSGSGSGTDFTLKISRVEAEDVGVYYCQQSRKVPSTFGQGTKLEIK

SEQ ID NO: 679 - protein, 107 aa, Homo sapiens [sequence omitted]

SEQ ID NO: 680 - protein, 8 aa, Homo sapiens
GYTFTDYA

SEQ ID NO: 681 - protein, 8 aa, Homo sapiens
IDTYYGNT

SEQ ID NO: 682 - protein, 10 aa, Homo sapiens
ARGGGDALDY

SEQ ID NO: 683 - protein, 6 aa, Homo sapiens
QSVSND

SEQ ID NO: 684 - protein, 3 aa, Homo sapiens
YAS

SEQ ID NO: 685 - protein, 9 aa, Homo sapiens
QQGYTSLRT

SEQ ID NO: 686 - protein, 117 aa, Homo sapiens
QVQLVQSGAEVKKPGASVKVSCKASGYTFTDYAMHWVRQAPGQGLEWMGVIDTYYGNTNYNQKFKGRVTMTRDTSISTAYMELSRLRSDDTAVYYCARGGGDALDYWGQGTTVTVSS

SEQ ID NO: 687 - protein, 330 aa, Homo sapiens [sequence omitted]

SEQ ID NO: 688 - protein, 107 aa, Homo sapiens
DIQMTQSPSSLSASVGDRVTITCKASQSVSNDVVWYQQKPGKAPKLLIYYASNRYIGVPSRFSGSGSGTDFTFTISSLQPEDIATYYCQQGYTSLRTFGQGTKLEIK

SEQ ID NO: 689 - protein, 107 aa, Homo sapiens [sequence omitted]

SEQ ID NO: 690 - protein, 9 aa, Homo sapiens
GYSITSGYS

SEQ ID NO: 691 - protein, 7 aa, Homo sapiens
IHYIGIT

SEQ ID NO: 692 - protein, 13 aa, Homo sapiens
AREDYDYDGVFAY

SEQ ID NO: 693 - protein, 6 aa, Homo sapiens
QSISNN

SEQ ID NO: 694 - protein, 3 aa, Homo sapiens
YAS

SEQ ID NO: 695 - protein, 9 aa, Homo sapiens
QQSNSWPHT

SEQ ID NO: 696 - protein, 120 aa, Homo sapiens
QVQLQESGPGLVKPSETLSLTCAVSGYSITSGYSWHWIRQPPGKGLEWIGYIHYIGITNNNPSLKSRVTISVDTSKNQFSLKLSSVTAADTAVYYCAREDYDYDGVFAYWGQGTLVTVSS

SEQ ID NO: 697 - protein, 330 aa, Homo sapiens [sequence omitted]

SEQ ID NO: 698 - protein, 107 aa, Homo sapiens
EIVLTQSPATLSLSPGERATLSCRASQSISNNLHWYQQKPGQAPRLLIYYASQSISGIPARFSGSGSGTDFTLTISSLEPEDFAVYYCQQSNSWPHTFGQGTKLEIK

SEQ ID NO: 699 - protein, 107 aa, Homo sapiens [sequence omitted]

SEQ ID NO: 700 - protein, 8 aa, Homo sapiens
GFSLTNYD

SEQ ID NO: 701 - protein, 7 aa, Homo sapiens
IWTGGGT

SEQ ID NO: 702 - protein, 10 aa, Homo sapiens
AREGLLLPLY

SEQ ID NO: 703 - protein, 6 aa, Homo sapiens
QNVGTN

SEQ ID NO: 704 - protein, 3 aa, Homo sapiens
SAS

SEQ ID NO: 705 - protein, 9 aa, Homo sapiens
QQYNSYPLT

SEQ ID NO: 706 - protein, 116 aa, Homo sapiens
QVQLQESGPGLVKPSETLSLTCTVSGFSLTNYDISWIRQPPGKGLEWIGVIWTGGGTNYNSAFMSRVTISVDTSKNQFSLKLSSVTAADTAVYYCAREGLLLPLYWGQGTLVTVSS

SEQ ID NO: 707 - protein, 330 aa, Homo sapiens [sequence omitted]

SEQ ID NO: 708 - protein, 107 aa, Homo sapiens
DIQMTQSPSSLSASVGDRVTITCKASQNVGTNVAWYQQKPGKVPKLLIYSASYRYSGVPSRFSGSGSGTDFTLTISSLQPEDVATYYCQQYNSYPLTFGQGTKLEIK

SEQ ID NO: 709 - protein, 107 aa, Homo sapiens [sequence omitted]

SEQ ID NO: 710 - protein, 8 aa, Homo sapiens
GYTFTNFG

SEQ ID NO: 711 - protein, 8 aa, Homo sapiens
INTYTGEP

SEQ ID NO: 712 - protein, 15 aa, Homo sapiens
ARGAYYYGSRVWFAY

SEQ ID NO: 713 - protein, 11 aa, Homo sapiens
QNLVHSNGNTY

SEQ ID NO: 714 - protein, 3 aa, Homo sapiens
KVS

SEQ ID NO: 715 - protein, 9 aa, Homo sapiens
SQSSHVPYT

SEQ ID NO: 716 - protein, 122 aa, Homo sapiens
QVQLVQSGSELKKPGASVKVSCKASGYTFTNFGMNWVRQAPGQGLEWMGWINTYTGEPTYADDFKGRFVFSLDTSVSTAYLQICSLKAEDTAVYYCARGAYYYGSRVWFAYWGQGTLVTVSS

SEQ ID NO: 717 - protein, 330 aa, Homo sapiens [sequence omitted]

SEQ ID NO: 718 - protein, 112 aa, Homo sapiens
DIVMTQSPLSLPVTPGEPASISCTSSQNLVHSNGNTYLHWYLQKPGQSPQLLIYKVSNRFSGVPDRFSGSGSGTDFTLKISRVEAEDVGVYYCSQSSHVPYTFGQGTKLEIK

SEQ ID NO: 719 - protein, 107 aa, Homo sapiens [sequence omitted]

SEQ ID NO: 720 - protein, 8 aa, Homo sapiens
GFSLTSSG

SEQ ID NO: 721 - protein, 7 aa, Homo sapiens
IWSGGNT

SEQ ID NO: 722 - protein, 13 aa, Homo sapiens
AREDYDYDWYFDV

SEQ ID NO: 723 - protein, 10 aa, Homo sapiens
ESVEYYGTSL

SEQ ID NO: 724 - protein, 3 aa, Homo sapiens
AAS

SEQ ID NO: 725 - protein, 9 aa, Homo sapiens
QQSRKVPWT

SEQ ID NO: 726 - protein, 119 aa, Homo sapiens
QVQLQESGPGLVKPSETLSLTCTVSGFSLTSSGVHWIRQPPGKGLEWIGVIWSGGNTDYNAAFISRVTISVDTSKNQFSLKLSSVTAADTAVYYCAREDYDYDWYFDVWGQGTTVTVSS

SEQ ID NO: 727 - protein, 330 aa, Homo sapiens [sequence omitted]

SEQ ID NO: 728 - protein, 111 aa, Homo sapiens
DIVMTQTPLSLSVTPGQPASISCRASESVEYYGTSLMQWYLQKPGQPPQLLIYAASNVKSGVPDRFSGSGSGTDFTLKISRVEAEDVGVYYCQQSRKVPWTFGQGTKLEIK

SEQ ID NO: 729 - protein, 107 aa, Homo sapiens [sequence omitted]

SEQ ID NO: 730 - protein, 8 aa, Homo sapiens
GYTFANFW

SEQ ID NO: 731 - protein, 8 aa, Homo sapiens
IFPGNSDT

SEQ ID NO: 732 - protein, 12 aa, Homo sapiens
ARELTGTYYFDY

SEQ ID NO: 733 - protein, 5 aa, Homo sapiens
SSVSY

SEQ ID NO: 734 - protein, 3 aa, Homo sapiens
SSS

SEQ ID NO: 735 - protein, 8 aa, Homo sapiens
HQWSGHFT

SEQ ID NO: 736 - protein, 119 aa, Homo sapiens
QVQLVQSGAEVKKPGASVKVSCKASGYTFANFWMHWVRQAPGQGLEWMGAIFPGNSDTGYNQKFKGRVTMTRDTSTSTVYMELSSLRSEDTAVYYCARELTGTYYFDYWGQGTLVTVSS

SEQ ID NO: 737 - protein, 330 aa, Homo sapiens [sequence omitted]

SEQ ID NO: 738 - protein, 105 aa, Homo sapiens
EIVLTQSPATLSLSPGERATLSCSATSSVSYMHWYQQKPGQAPRLLIYSSSNLASGIPARFSGSGSGTDFTLTISSLEPEDFAVYYCHQWSGHFTFGQGTKLEIK

SEQ ID NO: 739 - protein, 107 aa, Homo sapiens [sequence omitted]

SEQ ID NO: 740 - protein, 8 aa, Homo sapiens
GYTFANYL

SEQ ID NO: 741 - protein, 8 aa, Homo sapiens
IYPGGGFI

SEQ ID NO: 742 - protein, 13 aa, Homo sapiens
ARRFDYGGYFFDY

SEQ ID NO: 743 - protein, 11 aa, Homo sapiens
QSIVHSNGNIY

SEQ ID NO: 744 - protein, 3 aa, Homo sapiens
KVS

SEQ ID NO: 745 - protein, 9 aa, Homo sapiens
FQGSHVPWT

SEQ ID NO: 746 - protein, 120 aa, Homo sapiens; identical to SEQ ID NO: 10

SEQ ID NO: 747 - protein, 330 aa, Homo sapiens [sequence omitted]

SEQ ID NO: 748 - protein, 112 aa, Homo sapiens; identical to SEQ ID NO: 11

SEQ ID NO: 749 - protein, 107 aa, Homo sapiens [sequence omitted]

SEQ ID NO: 750 - protein, 8 aa, Homo sapiens
GFSITNYD

SEQ ID NO: 751 - protein, 7 aa, Homo sapiens
IWTGGGT

SEQ ID NO: 752 - protein, 12 aa, Homo sapiens
ARDRSPYFGYDY

SEQ ID NO: 753 - protein, 11 aa, Homo sapiens
QSLVHSNGNTY

SEQ ID NO: 754 - protein, 3 aa, Homo sapiens
KVS

SEQ ID NO: 755 - protein, 9 aa, Homo sapiens
SQSTHVPWT

SEQ ID NO: 756 - protein, 118 aa, Homo sapiens
QVQLQESGPGLVKPSETLSLTCTVSGFSITNYDISWIRQPPGKGLEWIGVIWTGGGTNYNSAFMSRVTISVDTSKNQFSLKLSSVTAADTAVYYCARDRSPYFGYDYWGQGTTVTVSS

SEQ ID NO: 757 - protein, 330 aa, Homo sapiens [sequence omitted]

SEQ ID NO: 758 - protein, 112 aa, Homo sapiens
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKLEIK

SEQ ID NO: 759 - protein, 107 aa, Homo sapiens [sequence omitted]

SEQ ID NO: 760 - protein, 9 aa, Homo sapiens
GYSITSDYA

SEQ ID NO: 761 - protein, 7 aa, Homo sapiens
MMYSGSA

SEQ ID NO: 762 - protein, 12 aa, Homo sapiens
ARFDHYYGRFDY

SEQ ID NO: 763 - protein, 5 aa, Homo sapiens
SSVSY

SEQ ID NO: 764 - protein, 3 aa, Homo sapiens
EIS

SEQ ID NO: 765 - protein, 9 aa, Homo sapiens
QQWNYPLFT

SEQ ID NO: 766 - protein, 119 aa, Homo sapiens
QVQLQESGPGLVKPSETLSLTCAVSGYSITSDYAWTWIRQPPGKGLEWIGYMMYSGSASYNPSLKGRVTISVDTSKNQFSLKLSSVTAADTAVYYCARFDHYYGRFDYWGQGTTVTVSS

SEQ ID NO: 767 - protein, 330 aa, Homo sapiens [sequence omitted]

SEQ ID NO: 768 - protein, 106 aa, Homo sapiens
AIQLTQSPSSLSASVGDRVTITCSASSSVSYIHWYQQKPGKAPKLLIYEISKLASGVPSRFSGSGSGTDFTLTISSLQPEDFATYYCQQWNYPLFTFGQGTKLEIK

SEQ ID NO: 769 - protein, 107 aa, Homo sapiens [sequence omitted]

SEQ ID NO: 770 - protein, 8 aa, Homo sapiens
GFTFRNYG

SEQ ID NO: 771 - protein, 8 aa, Homo sapiens
IISGGSYT

SEQ ID NO: 772 - protein, 15 aa, Homo sapiens
ARIYDHDGDYYAMDY

SEQ ID NO: 773 - protein, 11 aa, Homo sapiens
QSIVHSNGNTY

SEQ ID NO: 774 - protein, 3 aa, Homo sapiens
KVS

SEQ ID NO: 775 - protein, 9 aa, Homo sapiens
FQGSHVPWT

SEQ ID NO: 776 - protein, 122 aa, Homo sapiens
EVQLVESGGGLVQPGGSLRLSCAASGFTFRNYGMSWVRQAPGKGLEWVASIISGGSYTYYPDSVKGRFTISRDNAKNSLYLQMNSLRAEDTAVYYCARIYDHDGDYYAMDYWGQGTTVTVSS

SEQ ID NO: 777 - protein, 330 aa, Homo sapiens [sequence omitted]

SEQ ID NO: 778 - protein, 112 aa, Homo sapiens
DIVMTQSPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWTFGQGTKLEIK

SEQ ID NO: 779 - protein, 107 aa, Homo sapiens [sequence omitted]

SEQ ID NO: 780 - protein, 8 aa, Homo sapiens
GYTFTSYD

SEQ ID NO: 781 - protein, 8 aa, Homo sapiens
IYPGDGST

SEQ ID NO: 782 - protein, 10 aa, Homo sapiens
AREGITPFAY

SEQ ID NO: 783 - protein, 6 aa, Homo sapiens
KSISKY

SEQ ID NO: 784 - protein, 3 aa, Homo sapiens
SGS

SEQ ID NO: 785 - protein, 9 aa, Homo sapiens
QQHNEYPWT

SEQ ID NO: 786 - protein, 117 aa, Homo sapiens
QVQLVQSGAEVKKPGSSVKVSCKASGYTFTSYDINWVRQAPGQGLEWMGRIYPGDGSTKYNEKFKGRVTITADKSTSTAYMELSSLRSEDTAVYYCAREGITPFAYWGQGTTVTVSS

SEQ ID NO: 787 - protein, 330 aa, Homo sapiens [sequence omitted]

SEQ ID NO: 788 - protein, 107 aa, Homo sapiens
AIQLTQSPSSLSASVGDRVTITCRASKSISKYLAWYQQKPGKAPKLLIYSGSTLQSGVPSRFSGSGSGTDFTLTISSLQPEDFATYYCQQHNEYPWTFGQGTKLEIK

SEQ ID NO: 789 - protein, 107 aa, Homo sapiens [sequence omitted]

SEQ ID NO: 790 - protein, 8 aa, Homo sapiens
GGTFSSYA

SEQ ID NO: 791 - protein, 8 aa, Homo sapiens
IIPIFGTA

SEQ ID NO: 792 - protein, 13 aa, Homo sapiens
ARSSYGWSYEFDY

SEQ ID NO: 793 - protein, 6 aa, Homo sapiens
QSISSY

SEQ ID NO: 794 - protein, 3 aa, Homo sapiens
AAS

SEQ ID NO: 795 - protein, 9 aa, Homo sapiens
QQSYSTPLT

SEQ ID NO: 796 - protein, 120 aa, Homo sapiens; identical to SEQ ID NO: 226

SEQ ID NO: 797 - protein, 330 aa, Homo sapiens [sequence omitted]

SEQ ID NO: 798 - protein, 107 aa, Homo sapiens [sequence omitted]

SEQ ID NO: 799 - protein, 107 aa, Homo sapiens [sequence omitted]

SEQ ID NO: 800 - protein, 8 aa, Homo sapiens
GGTFSSYA

SEQ ID NO: 801 - protein, 8 aa, Homo sapiens
IIPIFGTA

SEQ ID NO: 802 - protein, 13 aa, Homo sapiens
ARSTVGWSYEFDY

SEQ ID NO: 803 - protein, 6 aa, Homo sapiens
QSISSY

SEQ ID NO: 804 - protein, 3 aa, Homo sapiens
AAS

SEQ ID NO: 805 - protein, 9 aa, Homo sapiens
QQSYSTPLT

SEQ ID NO: 806 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSTVGWSYEFDYWGQGTLVTVSS

SEQ ID NO: 807 - protein, 330 aa, Homo sapiens [sequence omitted]

SEQ ID NO: 808 - protein, 107 aa, Homo sapiens [sequence omitted]

SEQ ID NO: 809 - protein, 107 aa, Homo sapiens [sequence omitted]

SEQ ID NO: 810 - protein, 8 aa, Homo sapiens
GGTFSSYA

SEQ ID NO: 811 - protein, 8 aa, Homo sapiens
IIPIFGTA

SEQ ID NO: 812 - protein, 13 aa, Homo sapiens
ARHVLGWVLELDY

SEQ ID NO: 813 - protein, 6 aa, Homo sapiens
QSISSY

SEQ ID NO: 814 - protein, 3 aa, Homo sapiens
AAS

SEQ ID NO: 815 - protein, 9 aa, Homo sapiens
QQSYSTPLT

SEQ ID NO: 816 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHVLGWVLELDYWGQGTLVTVSS

SEQ ID NO: 817 - protein, 330 aa, Homo sapiens [sequence omitted]

SEQ ID NO: 818 - protein, 107 aa, Homo sapiens [sequence omitted]

SEQ ID NO: 819 - protein, 107 aa, Homo sapiens [sequence omitted]

SEQ ID NO: 820 - protein, 215 aa, Artificial Sequence
  light chain 767-IgG1.3
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 821 - protein, 452 aa, Artificial Sequence
  heavy chain 767-IgGl.3
EVQLVESGGGLVQPGKSLRLSCAASGFTLEDYAMHWVRQAPGKGLEWVSGIDWNSENIGYADSVKGRFTISRDNAKNSLYLQMNSLRTEDTALYYCAKVPGYSGGWIDAEDDWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 822 - protein, 215 aa, Artificial Sequence
  light chain 767-IgG1.3
EIVLTQSPGTLSLSPGERATSLCRASQSVSSSYLAWYQQKPGQAPRLLIYGASSRATGIPDRFSGSGSGTDFTLTISRLEPEDFAVYYCQQYGSSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 823 - protein, 452 aa, Artificial Sequence
  heavy chain 767-IgG1.3
EVQLVESGGGLVQPGKSLRLSCAASGFTLEDYAMHWVTQAPGKGLEWVSGIDWNSENIGYADSVKGRFTISRDNAKNSLYLQMNSLRTEDTALYYCAKVPGYSGGWIDAEDDWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 824 - protein, 120 aa, Artificial Sequence; identical to SEQ ID NO: 10
  VSTB92_VH

SEQ ID NO: 825 - protein, 112 aa, Artificial Sequence; identical to SEQ ID NO: 11
  VSTB92_VL

SEQ ID NO: 826 - protein, 330 aa, Artificial Sequence; identical to SEQ ID NO: 12
  IgG1_LALAw

SEQ ID NO: 827 - protein, 330 aa, Artificial Sequence; identical to SEQ ID NO: 13
  IgG1_INXLALA

SEQ ID NO: 828 - protein, 326 aa, Artificial Sequence; identical to SEQ ID NO: 14
  IgG2_sigma

SEQ ID NO: 829 - protein, 107 aa, Artificial Sequence [sequence omitted]

SEQ ID NO: 830 - protein, 450 aa, Artificial Sequence
  INX200_HC
QVQLVQSGAEVKKPGASVKVSCKASGYTFANYLIGWVRQAPGQRLEWMGDIYPGGGFISYNEKFKGRVTITRDTSASTAYMELSSLRSEDTAVYYCARRFDYGGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 831 - protein, 219 aa, Artificial Sequence
  INX200_LC
DIVMTQSPLSLPVTPGEPASISCRSSQSIVHSNGNIYLEWYLQKPGQSPQLLIYKVSNRFSGVPDRFSGSGSGTDFTLKISRVEAEDVGVYYCFQGSHVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 832 - protein, 452 aa, Artificial Sequence; identical to SEQ ID NO: 821
  767-IgG1.3_HC

SEQ ID NO: 833 - protein, 215 aa, Artificial Sequence; identical to SEQ ID NO: 820
  767-IgG1.3_LC

SEQ ID NO: 834 - protein, 450 aa, Artificial Sequence
  INX201_HC
QVQLVQSGAEVKKPGASVKVSCKASGYTFANYLIGWVRQAPGQRLEWMGDIYPGGGFISYNEKFKGRVTITRDTSASTAYMELSSLRSEDTAVYYCARRFDYGGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 835 - protein, 219 aa, Artificial Sequence; identical to SEQ ID NO: 831
  INX201_LC

SEQ ID NO: 836 - protein, 450 aa, Artificial Sequence
  INX231_HC
QVQLVQSGAEVKKPGASVKVSCKASGYTFTSYWLHWVRQAPGQGLEWMGYIIPNTLHTDYNQKFKDRVTMTRDTSTSTVYMELSSLRSEDTAVYYCARLDGDYDYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 837 - protein, 218 aa, Artificial Sequence
  INX231_LC
DIVMTQTPLSLSVTPGQPASISCRASESVEYYGTSFMQWYLQKPGQPPQLLIYTASNVESGVPDRFSGSGSGTDFTLKISRVEAEDVGVYYCQQSRKVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 838 - protein, 451 aa, Artificial Sequence
  INX234_HC
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDDALYGGYYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 839 - protein, 220 aa, Artificial Sequence
  INX234_LC
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 840 - protein, 449 aa, Artificial Sequence
  INX237_HC
QVQLVQSGAEVKKPGASVKVSCKASGYPFTGYFMHWVRQAPGQGLEWMGRINPYNGGTVYNQNFNDRVTMTRDTSTSTVYMELSSLRSEDTAVYYCARWTFDGLFMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 841 - protein, 218 aa, Artificial Sequence
  INX237_LC
DVVMTQSPLSLPVTLGQPASISCRASENVDKYGISFMNWFQQRPGQSPRRLIYATSNQGSGVPDRFSGSGSGTDFTLKISRVEAEDVGVYYCQQSKEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 842 - protein, 447 aa, Artificial Sequence
  INX240_HC
QVQLVQSGAEVKKPGASVKVSCKASGYTFTDYVISWVRQAPGQGLEWMGEIYPGSGNTYYYEKFKGRVTMTRDTSTSTVYMELSSLRSEDTAVYYCARVLVSVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 843 - protein, 213 aa, Artificial Sequence
  INX240_LC
EIVLTQSPDFQSVTPKEKVTITCSASSSINYITWYQQKPDQSPKLLIKDTSKLASGVPSRFSGSGSGTDFTLTINSLEAEDAATYYCHQRSSYPWTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0108119 A1
(43) Pub. Date: Apr. 3, 2025

(54) NOVEL STEROID PAYLOADS, STEROID LINKERS, ADCs CONTAINING AND USE THEREOF

(71) Applicant: IMMUNEXT, INC., Lebanon, NH (US)

(72) Inventors: Jay ROTHSTEIN, Norwich, VT (US); Kierstin BELL, Lebanon, NH (US); Catherine CARRIERE, Lebanon, NH (US); Michael MOLLOY, Enfield, NH (US); Anna KUTA, Lebanon, NH (US); Nicholas SCHWERTNER, Lebanon, NH (US); Maria DAy, Lebanon, NH (US); Xin Huang, Lebanon, NH (US); Dov PECHENICK, New Hampton, NH (US); Toni KLINE, Palo Alto, CA (US); Shibhani RAJANNA, Lebanon, NH (US); Yalin GUO, Lebanon, NH (US); Yingcai (Ian) WANG, Palo Alto, CA (US); Jieyu ZHOU, Bristol, PA (US); Sergey SEREGIN, Lebanon, NH (US); Erin CLARK, Lebanon, NH (US); Labros MEIMETIS, Stony Brook, NY (US); Julio MEDINA, South San Francisco, CA (US); Sheng Sun, Stamford, CT (US); Alexander KOVAL, Bristol, PA (US); Sravan THUMMANAPELLI, Yerdley, PA (US); Dmitry BORKIN, San Diego, CA (US); Rajeshkumar Maganlal LORIYA, San Diego, CA (US)

(21) Appl. No.: 18/271,106

(22) PCT Filed: Jan. 7, 2022
(86) PCT No.: PCT/US2022/011687

§ 371 Date: Jul. 6, 2023

Related U.S. Application Data
(60) Provisional application No. 63/290,100, filed on Dec. 16, 2021.
(60) Provisional application No. 63/284,886, filed on Dec. 1, 2021.
(60) Provisional application No. 63/271,554, filed on Oct. 25, 2021.
(60) Provisional application No. 63/271,023, filed on Oct. 22, 2021.
(60) Provisional application No. 63/251,939, filed on Oct. 4, 2021.
(60) Provisional application No. 63/246,941, filed on Sep. 22, 2021.
(60) Provisional application No. 63/188,499, filed on May 14, 2021.
(60) Provisional application No. 63/186,447, filed on May 10, 2021.
(60) Provisional application No. 63/178,378, filed on Apr. 22, 2021.
(60) Provisional application No. 63/138,958, filed on Jan. 19, 2021.
(60) Provisional application No. 63/134,811, filed on Jan. 7, 2021.

(51) Int. Cl. A61K 47/68; A61P 37/06; C07J 71/00; C07K 16/28

(52) U.S. Cl. CPC A61K 47/6803; A61K 47/6849; A61K 47/6889; A61P 37/06; C07J 71/0031; C07K 16/2827

(57) ABSTRACT

The invention provides novel glucocorticosteroids, glucocorticosteroid-linkers and antibody drug conjugates (ADC's) comprising an antibody or antibody fragment which binds to an antigen expressed on immune cells, optionally an antigen expressed on human immune cells. In some instances the ADCs comprise an anti-human VISTA (V-region Immunoglobulin-containing Suppressor of T cell Activation (1)) antibody or anti-VISTA antigen-binding antibody fragment which binds to VISTA expressing cells at physiologic pH having a short serum half-life (≈24-72 or 24-48 or 12-24 hours or less in a human VISTA knock-in rodent or ((≈1-3.5 days or less in a human or non-human primate). In some instances these ADCs have a rapid onset of action and are potent for prolonged duration as they are very effectively internalized by immune cells in large amounts where they are cleaved releasing large amounts of active steroid payload. The invention also relates to the use of such ADCs and novel steroids for the treatment of autoimmune, allergic and inflammatory conditions. The invention further relates to methods for reducing the adverse side effects and/or enhancing the efficacy of glucocorticoid receptor agonists by using such ADCs to selectively deliver these anti-inflammatory agents to target immune cells, such as monocytes, neutrophils, B cells, T cells, Tregs, cosinophils, NK cells, macrophages, myeloid cells, et al., and particularly myeloid cells, thereby reducing potential toxicity to non-target cells.

### CROSS-REFERENCE TO RELATED APPLICATIONS

This application is a U.S. National Phase application of Int'l Appl. No. PCT/US2022/011687, filed Jan. 7, 2022, which claims priority to U.S. Provisional Appl. No. 63/290,100, filed Dec. 16, 2021, U.S. Provisional Appl. No. 63/284,886, filed Dec. 1, 2021, U.S. Provisional Appl. No. 63/271,554, filed Oct. 25, 2021, U.S. Provisional Appl. No. 63/271,023, filed Oct. 22, 2021, U.S. Provisional Appl. No. 63/251,939, filed Oct. 4, 2021, U.S. Provisional Appl. No. 63/246,941, filed Sep. 22, 2021, U.S. Provisional Appl. No. 63/188,499, filed May 14, 2021, U.S. Provisional Appl. No. 63/186,447, filed May 10, 2021, U.S. Provisional Appl. No. 63/178,378, filed Apr. 22, 2021, U.S. Provisional Appl. No. 63/138,958, filed Jan. 19, 2021, U.S. Provisional Appl. No. 63/134,811, filed Jan. 7, 2021, each of which are incorporated herein by reference in their entireties.

### SEQUENCE DISCLOSURE

The instant application contains a Sequence Listing, which has been submitted in ASCII format via EFS-Web and is hereby incorporated by reference in its entirety. The ASCII copy, created on Jul. 5, 2023, is named “11432600008003.txt” and is 586,767 bytes in size.

### FIELD

The invention relates to novel glucocorticosteroids, glucocorticosteroid-linkers and antibody drug conjugates (ADC's) comprising an antibody or antibody fragment which binds to an antigen expressed on immune cells, typically an antigen expressed on human immune cells. In some embodiments the ADCs comprise an anti-human VISTA (V-region Immunoglobulin-containing Suppressor of T cell Activation (1)) antibody or anti-VISTA antigen-binding antibody fragment, e.g., one having a short serum half-life (˜ 24-27 hours or less in a human VISTA knock-in rodent). The subject ADCs have a rapid onset of action and are potent for prolonged duration as they are very effectively internalized by immune cells in large amounts where they are cleaved releasing large amounts of active steroid payload. The invention also relates to the use of such ADCs and novel steroids for the treatment of autoimmune, allergic, inflammatory and cancer conditions, and particularly acute and chronic autoimmune, allergic and inflammatory conditions. The invention further relates to methods for reducing the adverse side effects and/or enhancing the efficacy of glucocorticoids by using such ADCs to selectively deliver these anti-inflammatory agents to target immune cells, typically human immune cells, optionally any of monocytes, neutrophils, T cells, Tregs, eosinophils, B cells, NK cells, et al., and particularly myeloid cells, or other immune cells which are involved in the pathology of the treated autoimmune, allergic, inflammatory or cancer condition thereby reducing potential toxicity to non-target cells.

### BACKGROUND

VISTA is an NCR ligand, whose closest phylogenetic relative is PD-L1. VISTA bears homology to PD-L1 but displays a unique expression pattern that is restricted to the hematopoietic compartment. Specifically, VISTA is constitutively and highly expressed on CD11b high myeloid cells, and expressed at lower levels on CD4⁺ and CD8⁺ T cells. Like PD-L1, VISTA is a ligand that profoundly suppresses immunity, and like PD-L1, blocking VISTA allows for the development of therapeutic immunity to cancer in pre-clinical oncology models. Whereas blocking VISTA enhances immunity, especially CD8⁺ and CD4⁺ mediated T cell immunity, treatment with a soluble Ig fusion protein of the extracellular domain of VISTA (VISTA-Ig) suppresses immunity and has been shown to arrest the progression of multiple murine models of autoimmune disease. Based on the foregoing the use of antagonist anti-VISTA antibodies to promote T cell immunity and treat conditions where this is beneficial such as cancer and infection has been reported. Conversely the use of agonist anti-VISTA antibodies to inhibit T cell immunity and treat conditions where this is therapeutically beneficial such as autoimmune, allergic and inflammatory conditions has been reported. Unfortunately, some anti-VISTA antibodies including some which were used in human clinical trials possess a very short serum half-life which is generally undesirable in the context of treating chronic conditions such as cancer or autoimmunity as this necessitates very frequent dosing which is inconvenient for the patient as well as costly. Additionally, the potential usage of anti-VISTA antibodies and VISTA fusion proteins to deliver payloads such as chemotherapeutics to cancer cells or tumor sites has been suggested.

Synthetic glucocorticoid receptor agonists (e.g., dexamethasone, prednisolone, budesonide, beclomethasone, betamethasone, cortisol, cortisone acetate, 16-alpha hydroxyprednisolone, dexamethasone, difluorasone, flumethasone, flunisolide, fluocinolone acetonide, fluticasone propionate, ciclesonide, methylprednisolone, prednisone, prednisolone, mometasone, triamcinolone acetonide et al.) are a potent class of small molecules used in the treatment of inflammation and disorders associated therewith. While these compounds are very efficacious at inhibiting inflammation associated with different conditions such as autoimmune, allergic and inflammatory disorders, cancer and infectious diseases, their utility in the chronic treatment of inflammatory, allergic and autoimmune diseases is limited due to their severe side effects.

Based on the foregoing several approaches have been explored to retain the anti-inflammatory efficacy of synthetic glucocorticoids while sparing the unwanted toxicities have been described (Rosen, J and Miner, J N Endocrine Reviews 26:452-64 (2005)). In particular, antibody drug conjugates (ADCs) have been developed wherein such compounds are conjugated to antibodies which target antigens expressed by immune cells including CD40, CD163, CD74, PRLR and TNF. Notwithstanding, there is still a need in the field of autoimmune, allergic and inflammatory disease for improved anti-inflammatory, autoimmune and allergic therapies and the development of improved anti-inflammatory, autoimmune and allergic therapeutics, e.g., with enhanced efficacy, prolonged efficacy and/or reduced side effects compared to existing therapeutics for treatment of such conditions.

### SUMMARY

It is an object of the invention to provide therapeutics for treating or preventing inflammation and disorders associated therewith by providing novel steroids, steroid-linkers and ADCs which comprise an antibody or antibody fragment that targets an antigen expressed by immune cells, typically human immune cells and in some embodiments VISTA wherein the antibody or antibody fragment is an anti-human VISTA antibody or anti-human VISTA antibody fragment that binds to VISTA expressing cells at physiologic pH.

It is a specific object of the invention to provide novel antibody drug conjugates (ADC's) comprising an anti-VISTA antibody or antibody fragment which possesses a very short serum half-life at physiological conditions (≈pH 7.5), defined herein as 1 to 72 hours, 1 to 32 hours, 1 to 16 hours, 1 to 8 hours, 1 to 4 hours or 1-2 hours in a human VISTA knock-in rodent or ˜ 3-4 days or less in a Cynomolgus macaque, which anti-VISTA antibody or antibody fragment is conjugated to a glucocorticoid receptor agonist or glucocorticoid receptor agonist-linker disclosed herein.

As shown infra, the subject ADCs possess a unique combination of advantages over previous ADCs for targeting and directing internalization of anti-inflammatory agents, particularly steroids into immune cells, because of the novel properties of the steroid linker payload therein which provides for rapid internalization and release of large amounts of active payload once internalized by an immune cell.

Also, the subject ADCs provide for high drug antibody ratios (DARs) because they are less prone to aggregation compared to previous ADCs comprising glucocorticosteroids.

Also, the subject ADCs provide for high potency, even at lower DARs, because the subject ADCs are more effectively internalized and release more active glucocorticosteroid payload into target immune cells compared to previous ADCs comprising glucocorticosteroids.

Also, in some embodiments the subject ADCs possess the combined benefits of the steroid linker payloads disclosed herein and an anti-VISTA antibody or antibody fragment, particularly one that binds to VISTA expressing immune cells at physiologic pH and which possesses a very short pK. Particularly, these ADCs bind to immune cells which express VISTA, e.g., at very high density and notwithstanding their very short PK are efficacious (elicit anti-inflammatory activity) for prolonged duration (i.e., possess PDs much longer than their pk), and therefore are well suited for treating chronic inflammatory or autoimmune or allergic diseases wherein prolonged and repeated administration is therapeutically warranted.

Also, the subject ADCs which comprise anti-VISTA antibodies or antibody fragments, target a broad range of immune cells including activated and non-activated T cells, Tregs, CD4 T cells, CD8 T cells, neutrophils, myeloid, monocytes, macrophages, eosinophils, dendritic cells, NK cells, and endothelial cells; therefore such ADCs may be used to treat diseases inflammatory or autoimmune or allergic diseases involving any or all of these types of immune cells. However, alternatively, the subject ADCs may comprise antibodies or antibody fragments which bind to other immune cell antigens, preferably antibodies or antibody fragments which effectively internalize target immune cells.

The subject ADCs have a rapid onset of efficacy and therefore may be used to treat for acute treatment. In the case of VISTA antibody containing ADCs these ADCs do not bind B cells and therefore should not be as immunosuppressive as free steroids.

Also, in the case of VISTA antibody containing ADCs the subject ADCs act on Tregs which are an important immune cell responsible for steroid efficacy and act on both resting and such as myeloid cells, monocytes, eosinophils, Tregs, CD8 T cells, CD4 T cells, immune cells and consequently are active (elicit anti-inflammatory activity) both in active and remission phases of inflammatory and autoimmune conditions.

Also, in the case of VISTA antibody containing ADCs the subject ADCs act on neutrophils, which immune cells are critical for acute inflammation.

Also, in the case of VISTA antibody containing ADCs the subject ADCs internalize immune cells very rapidly and constitutively because VISTA cell surface turnover is high

Further, in the case of VISTA antibody containing ADCs the subject ADCs possess a very short half-life (PK) and selectively target immune cells, therefore the subject ADCs should not be prone to non-target cell related toxicities and undesired peripheral steroid exposure (low non-specific loss effects).

Also, in the case of some VISTA antibody containing ADCs according to the invention the subject ADCs' biological activity (anti-inflammatory action) is entirely attributable to the anti-inflammatory payload (steroid) because the anti-VISTA antibody is one possessing a silent IgG therein which elicits no immunological functions (no blocking of any VISTA biology).

It is a specific object of the invention to provide novel glucocorticoid agonist compounds having the following structure of Formula (I):

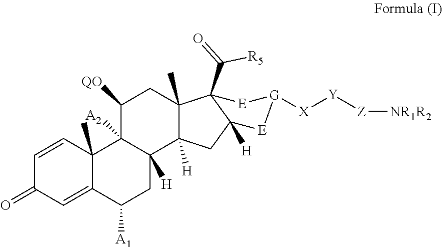

wherein X is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo[2.2.2]octane, adamantane, and cubane each of which can be substituted with 1-4 heteroatoms independently selected from F, Cl, Br, I, N, S, and O, each of which ring structure may contain at least one skeletal heteroatom selected from N, S, and O, and are optionally further substituted with 1-4 C1-3 alkyl or C1-3 perfluoroalkyl;
Z is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo[2.2.2]octane, adamantane, and cubane each of which can be substituted with 1-4 heteroatoms independently selected from F, Cl, Br, I, N, S, and O, each of which ring structure may contain at least one skeletal heteroatom selected from N, S, and O, and are optionally further substituted with 1-4 C1-3 alkyl or C1-3 perfluoroalkyl;
Y is selected from CHR1, O, S, and NR1;
E is selected from CH2 and O;
G is selected from CH, and N;
further wherein when G is CH and X is phenyl, Z is not phenyl;
the linkage of G to X may optionally be selected from C1-3 alkyl and ethylene oxide, each of which may be substituted with 1-4 heteroatoms independently selected from N, S, and O and are optionally further substituted with 1-4 C1-3 alkyl;
the linkage of X to Z may occupy any available position on X and Z;
substituent NR1R2 may occupy any available position on Z;
R1 is selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH2, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylaminoC(O)—;
when R1 is H, R2 may be selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH2, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylaminoC(O)—;
when R1 is H, linear or branched alkyl of 1-8 carbons, or heteroaryl, R2 may be a functional group selected from

[(C═O)CH(W)NH]m-[C═O]—[V]k-J,

(C═O)OCH2-p-aminophenyl-N—V-J,

(C═O)OCH2-p-aminophenyl-N—[(C═O)CH(W)NH]m-[C═O]—[V]k-J, and

[V]k-(C═O)OCH2-p-aminophenyl-N—[(C═O)CH(W)NH]m-[C═O]-J,
wherein m=1-6, k=0-1, and each permutation of W may independently be selected from H, [(CH2)nR3] where n=1-4, a branched alkyl chain terminating in R3, and a linear or branched polyethylene oxide group comprising 1-13 units;
R3 is selected from H, methyl, ethyl, isopropyl, OH, O-alkyl, NH2, NH-alkyl, N-dialkyl, SH, S-alkyl, guanidine, urea, carboxylic acid, carboxamide, carboxylic ester, substituted or unsubstituted aryl, substituted or unsubstituted heteroaryl, wherein said aryl and heteroaryl substituents may be selected from alkyl, haloalkyl, halogen, biphenyl, nitro, nitrile, —OH, —O-alkyl, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, and dialkylaminoC(O)—;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—;
alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH2, N3, thio, cyclooctyne, —OH, —CO2H, trans-cyclooctene, alkynyl, propargyl,

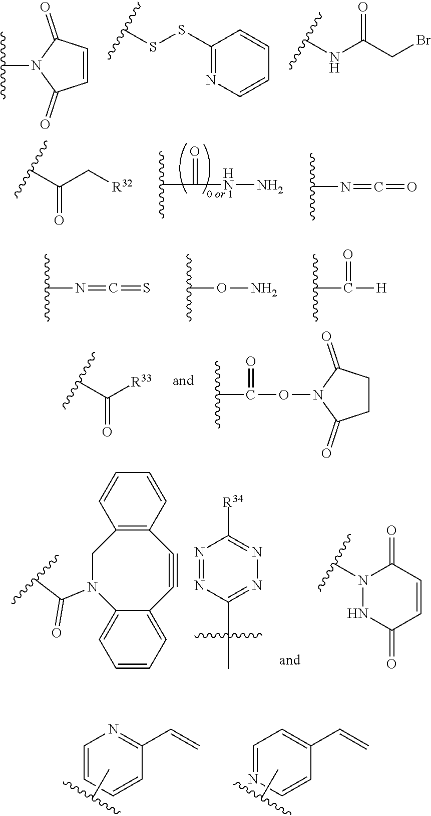

where R32 is selected from Cl, Br, F, mesylate, and tosylate and R33 is selected from Cl, Br, I, F, OH, —O—N-succinimidyl, —O-(4-nitrophenyl), —O-pentafluorophenyl or —O-tetrafluorophenyl R34 is H, Me, tetrazine-H, and tetrazine-Me;
R5 is selected from the group consisting of —CH₂OH, —CH2SH, —CH2Cl, —SCH2Cl, —SCH2F, —SCH2CF3, hydroxy, —OCH2CN, —OCH2Cl, —OCH2F, —OCH3, —OCH2CH3, —SCH2CN, and

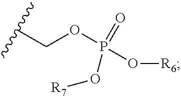

R6 and R7 are independently selected from hydrogen and C1-10 alkyl;
Q may be H,

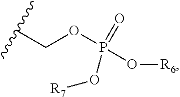

C(O)R8 where R8 is linear or branched alkyl of 1-8 carbons, or (C═O)NR4CHnNR4(C═O)OCH2-(V)n-J where n=1-4 and R4=H, alkyl or branched alkyl, or P(O)OR4;
A1 and A2 are independently selected from H and F; and
unless otherwise specified, all possible stereoisomers are claimed.

It is a specific object of the invention to provide a glucocorticoid agonist compound according to the foregoing, wherein X and Z are independently selected from phenyl, spiro[3.3]heptane, [1.1.1]bicyclopentane, and bicyclo[2.2.2]octane; Y is selected from CH₂ and O; permutations of W are independently selected from CH₂CH₂CO₂H and H, and further wherein when G is CH and X is phenyl, Z is not phenyl.

It is a specific object of the invention to provide a glucocorticoid agonist compound according to the foregoing, selected from any of the glucocorticoid agonist compounds disclosed in Example 3 or selected from those shown in FIG. 11 excluding INX J and INX L.

It is a specific object of the invention to provide a glucocorticoid agonist compound according to the foregoing, selected from the INX-steroid payloads, INX-steroid linkers and INX-antibody drug conjugate (ADC) compounds disclosed herein excluding INX J and INX L.

It is a specific object of the invention to provide a glucocorticoid agonist compound according to the foregoing, selected from the following:

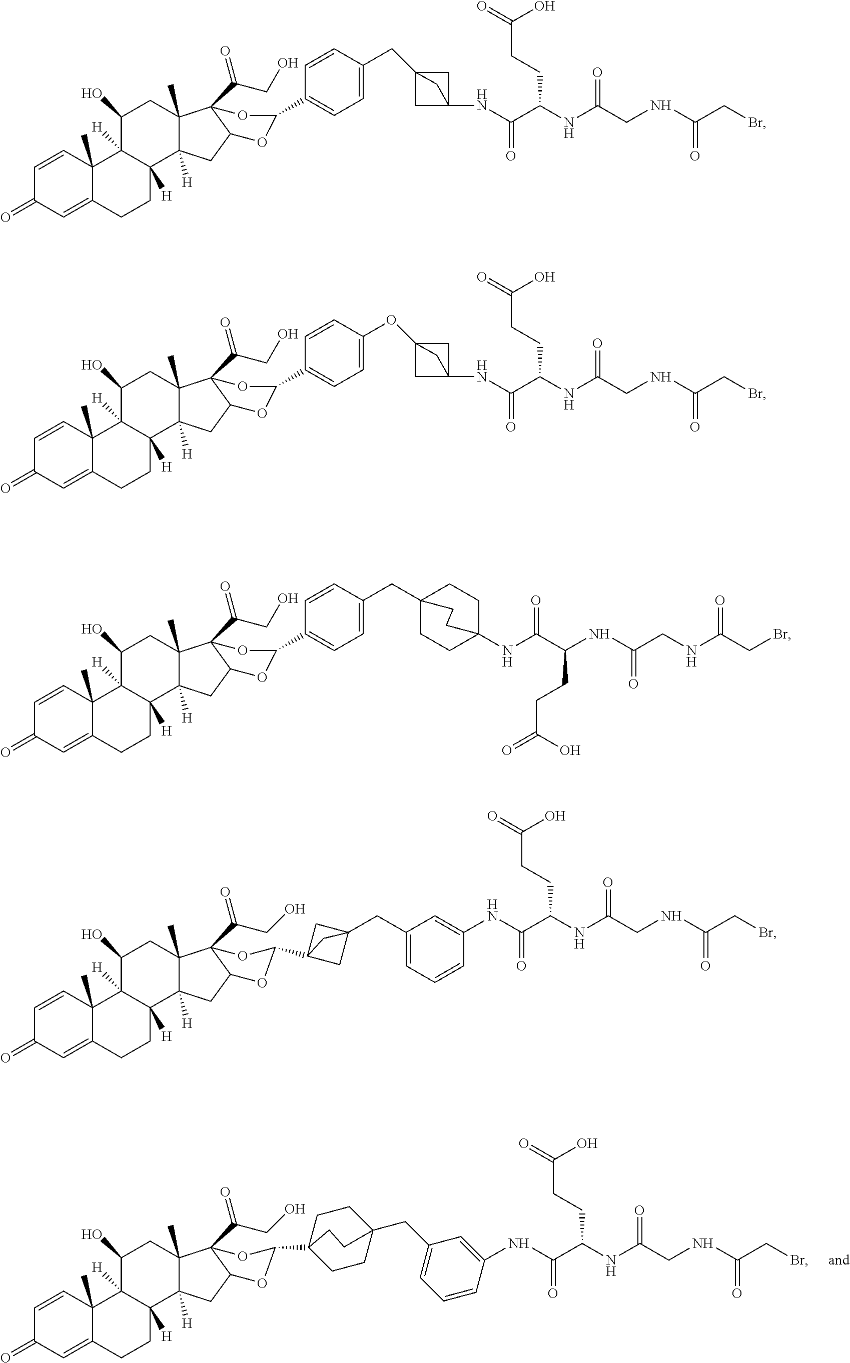

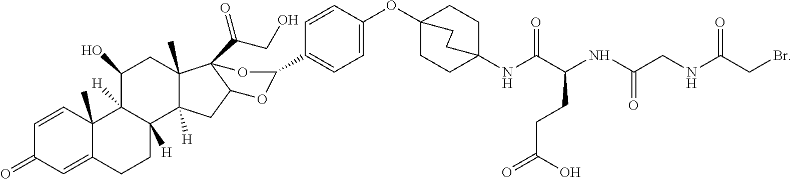

It is a specific object of the invention to provide a glucocorticoid agonist compound according to the foregoing, which is directly or indirectly attached to at least one cleavable or non-cleavable peptide and/or non-peptide linker (i.e., a “steroid-linker payload”). glucocorticoid agonist compound or steroid-linker payload.

It is a specific object of the invention to provide a compound (steroid-linker payload) that comprises at least one cleavable or non-cleavable linker (“L”), optionally “Q” a heterobifunctional group” or “heterotrifunctional group” which is a chemical moiety optionally used to connect the linker in the compound to an antibody or antibody fragment and at least one anti-inflammatory agent, (“AI”), wherein AI is a glucocorticoid agonist compound according to any of the foregoing which may be represented by the following structure:

Q-L-AI or AI-L-Q.

It is a specific object of the invention to provide a steroid-linker payload according to the foregoing, wherein the linker is selected from those disclosed herein.

It is a specific object of the invention to provide a steroid-linker payload according to any of the foregoing, comprising at least one cleavable or non-cleavable linker selected from PAB and/or an amino acid or a peptide, optionally 1-12 amino acids, further optionally dipeptide, a tripeptide, a quatrapeptide, a pentapeptide and further optionally Gly, Asn, Asp, Gln, Leu, Lys, Ala, Phe, Cit, Val, Val-Cit, Val-Ala, Val-Gly, Val-Gln, Ala-Val, Cit-Cit, Lys-Val-Cit, Asp-Val-Ala, Ala-Ala-Asn, Asp-Val-Ala, Ala-Val-Cit, Ala-Asn-Val, betaAla-Leu-Ala-Leu, Lys-Val-Ala, Val-Leu-Lys, Asp-Val-Cit, Val-Ala-Val, and Ala-Ala-Asn; or optionally at least one of GlcA, PAB, and Glu-Gly.

It is a specific object of the invention to provide a steroid-linker payload according to the foregoing, comprising at least one cleavable linker, and/or an immolative linker, is directly or indirectly attached to the glucocorticoid agonist steroid compound.

It is a more specific object of the invention to provide a glucocorticoid agonist steroid compound or steroid-linker payload or ADC containing according to any of the foregoing which is selected from any of the glucocorticoid agonist compounds or steroid-linker payload compounds disclosed in the examples, e.g., Example 3 and the compounds recited in FIG. 118A-O excluding INX J and INX L.

It is a specific object of the invention to provide a glucocorticoid agonist (Payload)-linker conjugate which is selected from:
(i) INX-SM-3-GluGly-Alkoxyamine, INX-SM-4-GluGly-Alkoxyamine, INX-SM-53-GluGly-Alkoxyamine, INX-SM-54-GluGly-Alkoxyamine, INX-SM-56-GluGly-Alkoxyamine, INX-SM-98-GluGly-Alkoxyamine, INX-SM-6-GluGly-Alkoxyamine, INX-SM-2-GluGly-Alkoxyamine, INX-SM-57-GluGly-Alkoxyamine, INX-SM-31-GluGly-Alkoxyamine, INX-SM-32-GluGly-Alkoxyamine, INX-SM-10-GluGly-Alkoxyamine, INX-SM-40-GluGly-Alkoxyamine, INX-SM-34-GluGly-Alkoxyamine, INX-SM-28-GluGly-Alkoxyamine, INX-SM-27-GluGly-Alkoxyamine, INX-SM-35-GluGly-Alkoxyamine, INX-SM-8-GluGly-Alkoxyamine, INX-SM-7-GluGly-Alkoxyamine, INX-SM-33-GluGly-Alkoxyamine or an glucocorticoid agonist (Payload)-linker conjugate wherein the Glu-Gly is substituted with a different cleavable peptide linker wherein another INX or INX-SM payload is substituted for the INX-SM payload comprised therein optionally selected from those in FIG. 118A-O; or
(ii) INX-SM-53-GluGly-Bromoacetyl, INX-SM-3-GluGly-Bromoacetyl, INX-SM-54-GluGly-Bromoacetyl, INX-SM-1-GluGly-Bromoacetyl, INX-SM-4-GluGly-Bromoacetyl, INX-SM-2-GluGly-Bromoacetyl, INX-SM-47-GluGly-Bromoacetyl, INX-SM-7-GluGly-Bromoacetyl, INX-SM-8-GluGly-Bromoacetyl, INX-SM-56-GluGly-Bromoacetyl, INX-SM-32-GluGly-Bromoacetyl, INX-SM-6-GluGly-Bromoacetyl, INX-SM-10-GluGly-Bromoacetyl, INX-SM-33-GluGly-Bromoacetyl, INX-SM-31-GluGly-Bromoacetyl, INX-SM-35-GluGly-Bromoacetyl, INX-SM-9-GluGly-Bromoacetyl, INX-SM-28-GluGly-Bromoacetyl, INX-SM-27-GluGly-Bromoacetyl, INX-SM-34-GluGly-Bromoacetyl, INX-SM-35-GluGly-Bromoacetyl, INX-SM-40-GluGly-Bromoacetyl or an glucocorticoid agonist (Payload)-linker conjugate wherein the Glu-Gly is substituted with a different cleavable peptide linker wherein another INX or INX-SM payload is substituted for the INX-SM payload comprised therein optionally selected from those in FIG. 118A-O;
(iii) INX-SM-53-GluGly-Dibenzocyclooctyne, INX-SM-1-GluGly-Dibenzocyclooctyne, INX-SM-4-GluGly-Dibenzocyclooctyne, INX-SM-54-GluGly-Dibenzocyclooctyne, INX-SM-7-GluGly-Dibenzocyclooctyne, INX-SM-8-GluGly-Dibenzocyclooctyne, INX-SM-2-GluGly-Dibenzocyclooctyne, INX-SM-57-GluGly-Dibenzocyclooctyne, INX-SM-40-GluGly-Dibenzocyclooctyne, INX-SM-34-GluGly-Dibenzocyclooctyne, INX-SM-28-GluGly-Dibenzocyclooctyne, INX-SM-27-GluGly-Dibenzocyclooctyne, INX-SM-35-GluGly-Dibenzocyclooctyne, INX-SM-9-GluGly-Dibenzocyclooctyne, INX-SM-10-GluGly-Dibenzocyclooctyne, INX-SM-31-GluGly-Dibenzocyclooctyne, INX-SM-32-GluGly-Dibenzocyclooctyne, INX-SM-33-GluGly-Dibenzocyclooctyne, INX-SM-56-GluGly-Dibenzocyclooctyne, INX-SM-6-GluGly-Dibenzocyclooctyne, INX-SM-3-GluGly-Dibenzocyclooctyne or an glucocorticoid agonist (Payload)-linker conjugate wherein the GluGly is substituted with a different cleavable peptide linker wherein another INX or INX-SM payload is substituted for the INX-SM payload comprised therein optionally selected from those in FIG. 118A-O; or
(iv) INX-SM-1-GluGly-NHS ester; INX-SM-31-GluGly-NHS ester; INX-SM-32-GluGly-NHS ester; INX-SM-33-GluGly-NHS ester; INX-SM-53-GluGly-NHS ester; INX-SM-7-GluGly-NHS ester; INX-SM-8-GluGly-NHS ester; INX-SM-2-GluGly-NHS ester; INX-SM-56-GluGly-NHS ester; INX-SM-6-GluGly-NHS ester; INX-SM-54-GluGly-NHS ester; INX-SM-4-GluGly-NHS ester; INX-SM-53-GluGly-NHS ester; INX-SM-3-GluGly-NHS ester; INX-SM-9-GluGly-NHS ester; INX-SM-40-GluGly-NHS ester; INX-SM-34-GluGly-NHS ester; INX-SM-28-GluGly-NHS ester; INX-SM-34-GluGly-NHS ester; INX-SM-28-GluGly-NHS ester; INX-SM-27-GluGly-NHS ester; INX-SM-35-GluGly-NHS ester; INX-SM-10-GluGly-NHS ester or an glucocorticoid agonist (Payload)-linker conjugate wherein the GluGly is substituted with a different cleavable peptide linker wherein another INX or INX-SM payload is substituted for the INX-SM payload comprised therein optionally selected from those in FIG. 118A-O;
(v) INX-SM-1-GluGly-Maleimide, INX-SM-3-GluGly-Maleimide, INX-SM-4-GluGly-Maleimide, INX-SM-8-GluGly-Maleimide, INX-SM-2-GluGly-Maleimide, INX-SM-7-GluGly-Maleimide, INX-SM-56-GluGly-Maleimide, INX-SM-6-GluGly-Maleimide, INX-SM-54-GluGly-Maleimide, INX-SM-53-GluGly-Maleimide, INX-SM-33-GluGly-Maleimide, INX-SM-35-GluGly-Maleimide, INX-SM-40-GluGly-Maleimide, INX-SM-34-GluGly-Maleimide, INX-SM-28-GluGly-Maleimide, INX-SM-27-GluGly-Maleimide, INX-SM-35-GluGly-Maleimide, INX-SM-9-GluGly-Maleimide, INX-SM-10-GluGly-Maleimide, INX-SM-31-GluGly-Maleimide, INX-SM-32-GluGly-Maleimide, INX-SM-57-GluGly-Maleimide or an glucocorticoid agonist (Payload)-linker conjugate wherein the GluGly is substituted with a different cleavable peptide linker wherein another INX or INX-SM payload is substituted for the INX-SM payload comprised therein optionally selected from those in FIG. 118A-O; or
(vi) INX-SM-3-GluGly-Tetrazine, INX-SM-53-GluGly-Tetrazine, INX-SM-1-GluGly-Tetrazine, INX-SM-54-GluGly-Tetrazine, INX-SM-6-GluGly-Tetrazine, INX-SM-56-GluGly-Tetrazine, INX-SM-4-GluGly-Tetrazine, INX-SM-10-GluGly-Tetrazine, INX-SM-31-GluGly-Tetrazine, INX-SM-32-GluGly-Tetrazine, INX-SM-33-GluGly-Tetrazine, INX-SM-7-GluGly-Tetrazine, INX-SM-8-GluGly-Tetrazine, INX-SM-9-GluGly-Tetrazine, INX-SM-27-GluGly-Tetrazine, INX-SM-35-GluGly-Tetrazine, INX-SM-2-GluGly-Tetrazine, INX-SM-40-GluGly-Tetrazine, INX-SM-34-GluGly-Tetrazine, INX-SM-28-GluGly-Tetrazine, INX-SM-27-GluGly-Tetrazine or an glucocorticoid agonist (Payload)-linker conjugate wherein the GluGly is substituted with a different cleavable peptide linker wherein another INX or INX-SM payload is substituted for the INX-SM payload comprised therein optionally selected from those in FIG. 118A-O; or
(vii) INX-SM-6-GluGly-Amine, INX-SM-54-GluGly-Amine, INX-SM-4-GluGly-Amine, INX-SM-53-GluGly-Amine, INX-SM-2-GluGly-Amine, INX-SM-56-GluGly-Amine, INX-SM-57-GluGly-Amine, INX-SM-35-GluGly-Amine, INX-SM-27-GluGly-Amine, INX-SM-40-GluGly-Amine, INX-SM-34-GluGly-Amine, INX-SM-28-GluGly-Amine, INX-SM-35-GluGly-Amine, INX-SM-9-GluGly-Amine, INX-SM-10-GluGly-Amine, INX-SM-31-GluGly-Amine, INX-SM-32-GluGly-Amine, INX-SM-33-GluGly-Amine, INX-SM-7-GluGly-Amine, INX-SM-8-GluGly-Amine, INX-SM-1-GluGly-Amine, INX-SM-3-GluGly-Amine or an glucocorticoid agonist (Payload)-linker conjugate wherein the GluGly is substituted with a different cleavable peptide linker wherein another INX or INX-SM payload is substituted for the INX-SM payload comprised therein optionally selected from those in FIG. 118A-O; or
(viii) INX-SM-53-PAB-GluGly-Alkoxyamine, INX-SM-1-PAB-GluGly-Alkoxyamine, INX-SM-3-PAB-GluGly-Alkoxyamine, INX-SM-2-PAB-GluGly-Alkoxyamine, INX-SM-56-PAB-GluGly-Alkoxyamine, INX-SM-35-PAB-GluGly-Alkoxyamine, INX-SM-25-PAB-GluGly-Alkoxyamine, INX-SM-27-PAB-GluGly-Alkoxyamine, INX-SM-35-PAB-GluGly-Alkoxyamine, INX-SM-9-PAB-GluGly-Alkoxyamine, INX-SM-10-PAB-GluGly-Alkoxyamine, INX-SM-31-PAB-GluGly-Alkoxyamine, INX-SM-32-PAB-GluGly-Alkoxyamine, INX-SM-33-PAB-GluGly-Alkoxyamine, INX-SM-57-PAB-GluGly-Alkoxyamine, INX-SM-7-PAB-GluGly-Alkoxyamine, INX-SM-8-PAB-GluGly-Alkoxyamine, INX-SM-6-PAB-GluGly-Alkoxyamine, INX-SM-54-PAB-GluGly-Alkoxyamine, INX-SM-4-PAB-GluGly-Alkoxyamine, INX-SM-40-PAB-GluGly-Alkoxyamine, INX-SM-34-PAB-GluGly-Alkoxyamine or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O; or
(ix) INX-SM-1-PAB-GluGly-Bromoacetyl, INX-SM-3-PAB-GluGly-Bromoacetyl, INX-SM-2-PAB-GluGly-Bromoacetyl, INX-SM-7-PAB-GluGly-Bromoacetyl, INX-SM-8-PAB-GluGly-Bromoacetyl, INX-SM-40-PAB-GluGly-Bromoacetyl, INX-SM-56-PAB-GluGly-Bromoacetyl, INX-SM-6-PAB-GluGly-Bromoacetyl, INX-SM-154PAB-GluGly-Bromoacetyl, INX-SM-4-PAB-GluGly-Bromoacetyl, INX-SM-33-PAB-GluGly-Bromoacetyl, INX-PAB-GluGly-Bromoacetyl, INX-SM-32-PAB-GluGly-Bromoacetyl, INX-SM-10-PAB-GluGly-Bromoacetyl, INX-SM-34-PAB-GluGly-Bromoacetyl, INX-SM-31-PAB-GluGly-Bromoacetyl, INX-SM-9-PAB-GluGly-Bromoacetyl, INX-SM-28-PAB-GluGly-Bromoacetyl, INX-SM-27-PAB-GluGly-Bromoacetyl, INX-SM-35-PAB-GluGly-Bromoacetyl, INX-SM-53-PAB-GluGly-Bromoacetyl or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O;
(x) INX-SM-6-PAB-GluGly-Dibenzocyclooctyne, INX-SM-54-PAB-GluGly-Dibenzocyclooctyne,
INX-SM-4-PAB-GluGly-Dibenzocyclooctyne, INX-SM-53-PAB-GluGly-Dibenzocyclooctyne, INX-SM-1-PAB-GluGly-Dibenzocyclooctyne, INX-SM-7-PAB-GluGly-Dibenzocyclooctyne, INX-SM-8-PAB-GluGly-Dibenzocyclooctyne, INX-SM-2-PAB-GluGly-Dibenzocyclooctyne, INX-SM-56-PAB-GluGly-Dibenzocyclooctyne, INX-SM-57-PAB-GluGly-Dibenzocyclooctyne, INX-SM-33-PAB-GluGly-Dibenzocyclooctyne, INX-SM-32-PAB-GluGly-Dibenzocyclooctyne, INX-SM-31-PAB-GluGly-Dibenzocyclooctyne, INX-SM-3-PAB-GluGly-Dibenzocyclooctyne, INX-SM-9-PAB-GluGly-Dibenzocyclooctyne, INX-SM-27-PAB-GluGly-Dibenzocyclooctyne, INX-SM-35-PAB-GluGly-Dibenzocyclooctyne, INX-SM-34-PAB-GluGly-Dibenzocyclooctyne, INX-SM-28-PAB-GluGly-Dibenzocyclooctyne, INX-SM-40-PAB-GluGly-Dibenzocyclooctyne, INX-SM-10-PAB-GluGly-Dibenzocyclooctyne or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O; or
(xi) INX-SM-56-PAB-GluGly-NHS ester, INX-SM-54-PAB-GluGly-NHS ester, INX-SM-4-PAB-GluGly-NHS ester, INX-SM-53-PAB-GluGly-NHS ester, INX-SM-1-PAB-GluGly-NHS ester, INX-SM-3-PAB-GluGly-NHS ester, INX-SM-33-PAB-GluGly-NHS ester, INX-SM-57-PAB-GluGly-NHS ester, INX-SM-7-PAB-GluGly-NHS ester, INX-SM-8-PAB-GluGly-NHS ester, INX-SM-27-PAB-GluGly-NHS ester, INX-SM-35-PAB-GluGly-NHS ester, INX-SM-9-PAB-GluGly-NHS ester, INX-SM-10-PAB-GluGly-NHS ester, INX-SM-31-PAB-GluGly-NHS ester, INX-SM-32-PAB-GluGly-NHS ester, INX-SM-40-PAB-GluGly-NHS ester, INX-SM-34-PAB-GluGly-NHS ester, INX-SM-28-PAB-GluGly-NHS ester, INX-SM-2-PAB-GluGly-NHS ester or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O; or
(xii) INX-SM-1-PAB-GluGly-Maleimide, INX-SM-53-PAB-GluGly-Maleimide, INX-SM-5-PAB-GluGly-Maleimide, INX-SM-2-PAB-GluGly-Maleimide, INX-SM-8-PAB-GluGly-Maleimide, INX-SM-56-PAB-GluGly-Maleimide, INX-SM-54-PAB-GluGly-Maleimide, INX-SM-4-PAB-GluGly-Maleimide, INX-SM-57-PAB-GluGly-Maleimide, INX-SM-7-PAB-GluGly-Maleimide, INX-SM-32-PAB-GluGly-Maleimide, INX-SM-31-PAB-GluGly-Maleimide, INX-SM-53-PAB-GluGly-Maleimide, INX-SM-3-PAB-GluGly-Maleimide, INX-SM-34-PAB-GluGly-Maleimide, INX-SM-28-PAB-GluGly-Maleimide, INX-SM-40-PAB-GluGly-Maleimide, INX-SM-27-PAB-GluGly-Maleimide, INX-SM-35-PAB-GluGly-Maleimide, INX-SM-9-PAB-GluGly-Maleimide, INX-SM-10-PAB-GluGly-Maleimide or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O; or
(xiii) INX-SM-6-PAB-GluGly-Tetrazine, INX-SM-54-PAB-GluGly-Tetrazine, INX-SM-4-PAB-GluGly-Tetrazine, INX-SM-53-PAB-GluGly-Tetrazine, INX-SM-1-PAB-GluGly-Tetrazine, INX-SM-3-PAB-GluGly-Tetrazine, INX-SM-57-PAB-GluGly-Tetrazine, INX-SM-7-PAB-GluGly-Tetrazine, INX-SM-8-PAB-GluGly-Tetrazine, INX-SM-2-PAB-GluGly-Tetrazine, INX-SM-31-PAB-GluGly-Tetrazine, INX-SM-32-PAB-GluGly-Tetrazine, INX-SM-33-PAB-GluGly-Tetrazine, INX-SM-56-PAB-GluGly-Tetrazine, INX-SM-35-PAB-GluGly-Tetrazine, INX-SM-9-PAB-GluGly-Tetrazine, INX-SM-40-PAB-GluGly-Tetrazine, INX-SM-34-PAB-GluGly-Tetrazine, INX-SM-28-PAB-GluGly-Tetrazine, INX-SM-27-PAB-GluGly-Tetrazine, INX-SM-35-PAB-GluGly-Tetrazine, INX-SM-10-PAB-GluGly-Tetrazine or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O; or
(xiv) INX-SM-1-PAB-GluGly-Amine, INX-SM-3-PAB-GluGly-Amine, INX-SM-8-PAB-GluGly-Amine, INX-SM-2-PAB-GluGly-Amine, INX-SM-56-PAB-GluGly-Amine, INX-SM-6-PAB-GluGly-Amine, INX-SM-54-PAB-GluGly-Amine, INX-SM-4-PAB-GluGly-Amine, INX-SM-53-PAB-GluGly-Amine, INX-SM-33-PAB-GluGly-Amine, INX-SM-53-PAB-GluGly-Amine, INX-SM-7-PAB-GluGly-Amine, INX-SM-9-PAB-GluGly-Amine, INX-SM-35-PAB-GluGly-Amine, INX-SM-40-PAB-GluGly-Amine, INX-SM-34-PAB-GluGly-Amine, INX-SM-28-PAB-GluGly-Amine, INX-SM-27-PAB-GluGly-Amine, INX-SM-35-PAB-GluGly-Amine, INX-SM-10-PAB-GluGly-Amine, INX-SM-31-PAB-GluGly-Amine, INX-SM-32-PAB-GluGly-Amine or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O; or
(xv) INX-SM-1-PAB-GlcA-Alkoxyamine, INX-SM-35-PAB-GlcA-Alkoxyamine, INX-SM-9-PAB-GlcA-Alkoxyamine, INX-SM-10-PAB-GlcA-Alkoxyamine, INX-SM-54-PAB-GlcA-Alkoxyamine, INX-SM-31-PAB-GlcA-Alkoxyamine, INX-SM-32-PAB-GlcA-Alkoxyamine, INX-SM-33-PAB-GlcA-Alkoxyamine, INX-SM-57-PAB-GlcA-Alkoxyamine, INX-SM-7-PAB-GlcA-Alkoxyamine, INX-SM-8-PAB-GlcA-Alkoxyamine, INX-SM-2-PAB-GlcA-Alkoxyamine, INX-SM-56-PAB-GlcA-Alkoxyamine, INX-SM-6-PAB-GlcA-Alkoxyamine, INX-SM-4-PAB-GlcA-Alkoxyamine, INX-SM-53-PAB-GlcA-Alkoxyamine, INX-SM-27-PAB-GlcA-Alkoxyamine, INX-SM-40-PAB-GlcA-Alkoxyamine, INX-SM-34-PAB-GlcA-Alkoxyamine, INX-SM-28-PAB-GlcA-Alkoxyamine, INX-SM-3-PAB-GlcA-Alkoxyamine or another glucocorticoid agonist (Payload)-linker conjugate wherein the GlcA and/or the PAB is substituted with a different cleavable peptide or non-peptide linker wherein another INX or INX-SM payload is substituted for the INX-SM payload comprised therein optionally selected from those in FIG. 118A-O; or
(xvi) INX-SM-3-PAB-GlcA-Bromoacetyl, INX-SM-4-PAB-GlcA-Bromoacetyl, INX-SM-56-PAB-GlcA-Bromoacetyl, INX-SM-54-PAB-GlcA-Bromoacetyl, INX-SM-4-PAB-GlcA-Bromoacetyl, INX-SM-53-PAB-GlcA-Bromoacetyl, INX-SM-7-PAB-GlcA-Bromoacetyl, INX-SM-8-PAB-GlcA-Bromoacetyl, INX-SM-2-PAB-GlcA-Bromoacetyl, INX-SM-40-PAB-GlcA-Bromoacetyl, INX-SM-57-PAB-GlcA-Bromoacetyl, INX-SM-33-PAB-GlcA-Bromoacetyl, INX-SM-10-PAB-GlcA-Bromoacetyl, INX-SM-34-PAB-GlcA-Bromoacetyl, INX-SM-31-PAB-GlcA-Bromoacetyl, INX-SM-32-PAB-GlcA-Bromoacetyl, INX-SM-35-PAB-GlcA-Bromoacetyl, INX-SM-9-PAB-GlcA-Bromoacetyl, INX-SM-28-PAB-GlcA-Bromoacetyl, INX-SM-27-PAB-GlcA-Bromoacetyl, INX-SM-1-PAB-GlcA-Bromoacetyl or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O; or
(xvii) INX-SM-4-PAB-GlcA-Dibenzocyclooctyne, INX-SM-54-PAB-GlcA-Dibenzocyclooctyne, INX-SM-1-PAB-GlcA-Dibenzocyclooctyne, INX-SM-54-PAB-GlcA-Dibenzocyclooctyne, INX-SM-33-PAB-GlcA-Dibenzocyclooctyne, INX-SM-57-PAB-GlcA-Dibenzocyclooctyne, INX-SM-7-PAB-GlcA-Dibenzocyclooctyne, INX-SM-8-PAB-GlcA-Dibenzocyclooctyne, INX-SM-2-PAB-GlcA-Dibenzocyclooctyne, INX-SM-5-PAB-GlcA-Dibenzocyclooctyne, INX-SM-6-PAB-GlcA-Dibenzocyclooctyne, INX-SM-35-PAB-GlcA-Dibenzocyclooctyne, INX-SM-9-PAB-GlcA-Dibenzocyclooctyne, INX-SM-10-PAB-GlcA-Dibenzocyclooctyne, INX-SM-31-PAB-GlcA-Dibenzocyclooctyne, INX-SM-32-PAB-GlcA-Dibenzocyclooctyne, INX-SM-27-PAB-GlcA-Dibenzocyclooctyne, INX-SM-35-PAB-GlcA-Dibenzocyclooctyne, INX-SM-28-PAB-GlcA-Dibenzocyclooctyne, INX-SM-34-PAB-GlcA-Dibenzocyclooctyne, INX-SM-40-PAB-GlcA-Dibenzocyclooctyne, INX-SM-3-PAB-GlcA-Dibenzocyclooctyne or another glucocorticoid agonist (Payload)-linker conjugate wherein the GlcA and/or the PAB linker is substituted with a different cleavable peptide or non-peptide linker wherein another INX or INX-SM payload is substituted for the INX-SM payload comprised therein optionally selected from those in FIG. 118A-O; or
(xviii) INX-SM-3-PAB-GlcA-NHS Ester, INX-SM-53-PAB-GlcA-NHS Ester, INX-SM-4-PAB-GlcA-NHS Ester, INX-SM-56-PAB-GlcA-NHS Ester, INX-SM-54-PAB-GlcA-NHS Ester, INX-SM-8-PAB-GlcA-NHS Ester, INX-SM-2-PAB-GlcA-NHS Ester, INX-SM-7-PAB-GlcA-NHS Ester, INX-SM-57-PAB-GlcA-NHS Ester, INX-SM-32-PAB-GlcA-NHS Ester, INX-SM-33-PAB-GlcA-NHS Ester, INX-SM-31-PAB-GlcA-NHS Ester, INX-SM-9-PAB-GlcA-NHS Ester, INX-SM-10-PAB-GlcA-NHS Ester, INX-SM-35-PAB-GlcA-NHS Ester, INX-SM-27-PAB-GlcA-NHS Ester, INX-SM-28-PAB-GlcA-NHS Ester, INX-SM-40-PAB-GlcA-NHS Ester, INX-SM-34-PAB-GlcA-NHS Ester, INX-SM-1-PAB-GlcA-NHS Ester or another glucocorticoid agonist (Payload)-linker conjugate wherein the GlcA and/or the PAB is substituted with a different cleavable peptide or non-peptide linker wherein another INX or INX-SM payload is substituted for the INX-SM payload comprised therein optionally selected from those in FIG. 118A-O; or
(xix) INX-SM-3-PAB-GlcA-Maleimide, INX-SM-4-PAB-GlcA-Maleimide, INX-SM-53-PAB-GlcA-Maleimide, INX-SM-31-PAB-GlcA-Maleimide, INX-SM-32-PAB-GlcA-Maleimide, INX-SM-33-PAB-GlcA-Maleimide, INX-SM-53-PAB-GlcA-Maleimide, INX-SM-7-PAB-GlcA-Maleimide, INX-SM-8-PAB-GlcA-Maleimide, INX-SM-2-PAB-GlcA-Maleimide, INX-SM-56-PAB-GlcA-Maleimide, INX-SM-6-PAB-GlcA-Maleimide, INX-SM-54-PAB-GlcA-Maleimide, INX-SM-1-PAB-GlcA-Maleimide, INX-SM-9-PAB-GlcA-Maleimide, INX-SM-35-PAB-GlcA-Maleimide, INX-SM-27-PAB-GlcA-Maleimide, INX-SM-28-PAB-GlcA-Maleimide, INX-SM-34-PAB-GlcA-Maleimide, INX-SM-40-PAB-GlcA-Maleimide, INX-SM-10-PAB-GlcA-Maleimide or another glucocorticoid agonist (Payload)-linker conjugate wherein the GlcA and/or the PAB is substituted with a different cleavable peptide or non-peptide linker wherein another INX or INX-SM payload is substituted for the INX-SM payload comprised therein optionally selected from those in FIG. 118A-O; or
(xx) INX-SM-33-PAB-GlcA-Tetrazine, INX-SM-57-PAB-GlcA-Tetrazine, INX-SM-7-PAB-GlcA-Tetrazine, INX-SM-8-PAB-GlcA-Tetrazine, INX-SM-2-PAB-GlcA-Tetrazine, INX-SM-56-PAB-GlcA-Tetrazine, INX-SM-6-PAB-GlcA-Tetrazine, INX-SM-54-PAB-GlcA-Tetrazine, INX-SM-4-PAB-GlcA-Tetrazine, INX-SM-9-PAB-GlcA-Tetrazine, INX-SM-35-PAB-GlcA-Tetrazine, INX-SM-27-PAB-GlcA-Tetrazine, INX-SM-28-PAB-GlcA-Tetrazine, INX-SM-34-PAB-GlcA-Tetrazine, INX-SM-40-PAB-GlcA-Tetrazine, INX-SM-10-PAB-GlcA-Tetrazine or another glucocorticoid agonist (Payload)-linker conjugate wherein the GlcAy and/or the PAB linker is substituted with a different cleavable peptide or non-peptide linker wherein another INX or INX-SM payload is substituted for the INX-SM payload comprised therein optionally selected from those in FIG. 118A-O; or
(xxi) INX-SM-1-PAB-GlcA-Amine, INX-SM-3-PAB-GlcA-Amine, INX-SM-53-PAB-GlcA-Amine, INX-SM-6-PAB-GlcA-Amine, INX-SM-54-PAB-GlcA-Amine, INX-SM-8-PAB-GlcA-Amine, INX-SM-2-PAB-GlcA-Amine, INX-SM-56-PAB-GlcA-Amine, INX-SM-4-PAB-GlcA-Amine, INX-SM-35-PAB-GlcA-Amine, INX-SM-8-PAB-GlcA-Amine, INX-SM-10-PAB-GlcA-Amine, INX-SM-31-PAB-GlcA-Amine, INX-SM-32-PAB-GlcA-Amine, INX-SM-33-PAB-GlcA-Amine, INX-SM-57-PAB-GlcA-Amine, INX-SM-27-PAB-GlcA-Amine, INX-SM-35-PAB-GlcA-Amine, INX-SM-34-PAB-GlcA-Amine, INX-SM-28-PAB-GlcA-Amine, INX-SM-40-PAB-GlcA-Amine, INX-SM-7-PAB-GlcA-Amine or another glucocorticoid agonist (Payload)-linker conjugate wherein the GlcA and/or the PAB linker is substituted with a different cleavable peptide or non-peptide linker wherein another INX or INX-SM payload is substituted for the INX-SM payload comprised therein optionally selected from those in FIG. 118A-O; or
(xxii) Alkoxyamine-GlcA-PAB-DMEDA-INX-SM3, or Alkoxyamine-GlyGlu-PAB-DMEDA-INX-SM3, or other linker payloads comprising the same or different peptide or non-peptide linkers wherein the linker is attached to the same or different INX Steroid via the C11-OH;
(xxiii) Bromoacetyl-GlcA-PAB-DMEDA-INX-SM3, or Bromoacetyl-GlyGlu-PAB-DMEDA-INX-SM3, or other linker payloads comprising the same or different peptide or non-peptide linkers wherein the linker is attached to the same or different INX Steroid via the C11-OH;
(xxiv) Dibenzocyclooctyne-GlcA-PAB-DMEDA-INX-SM3, or Dibenzocyclooctyne-GlyGlu-PAB-DMEDA-INX-SM3, or other linker payloads comprising the same or different peptide or non-peptide linkers wherein the linker is attached to the same or different INX Steroid via the C11-OH;
(xxv) Tetrazine-GlcA-PAB-DMEDA-INX-SM3, or Tetrazine-GlyGlu-PAB-DMEDA-INX-SM3, or other linker payloads comprising the same or different peptide or non-peptide linkers wherein the linker is attached to the same or different INX Steroid via the C11-OH;
(xxvi) Alkoxyamine-GlcA-PAB-DMEDA-INX-SM3, or Alkoxyamine-GlyGlu-PAB-DMEDA-INX-SM3, or other linker payloads comprising the same or different linkers wherein a linker is attached to the same or different INX Steroid payload via C17;
(xxvii) Bromoacetyl-GlcA-PAB-DMEDA-INX-SM3, or Bromoacetyl-GlyGlu-PAB-DMEDA-INX-SM3, or other linker payloads comprising the same or different linker wherein a linker is attached to the same or different INX Steroid payload via C17;
(xxviii) Maleimide-GlcA-PAB-DMEDA-INX-SM3, or Maleimide-GlyGlu-PAB-DMEDA-INX-SM3, or other linker payloads comprising the same or different linker wherein the linker is attached to the same or different INX Steroid payload via C17;
(xxix) Dibenzocyclooctyne-GlcA-PAB-DMEDA-INX-SM3, or Dibenzocyclooctyne-GlyGlu-PAB-DMEDA-INX-SM3, or other linker payloads comprising the same or different linker wherein the linker is attached to the same or different INX Steroid payload via C17;
(xxx) Tetrazine-GlcA-PAB-DMEDA-INX-SM3, or Tetrazine-GlyGlu-PAB-DMEDA-INX-SM3, or other linker payloads comprising the same or different linker wherein the linker is attached to the same or different INX Steroid payload via C17; and
(xxxi) Amine-GlcA-PAB-DMEDA-INX-SM3, or Amine-GlyGlu-PAB-DMEDA-INX-SM3, or other linker payloads comprising the same or different linker wherein the linker is attached to the same or different INX Steroid payload via C17.

13. An antibody drug conjugate (ADC) selected from the following:
Ab-Gly-Glu-PAB-DMEDA-INX-3 or Ab-GlcA-PAB-DMEDA-INX-SM-3 (alkoxyamine+ketone conjugation (C11-OH linked), or another ADC comprising a different INX-SM payload wherein the INX-SM payload is conjugated to the antibody via alkoxyamine+ketone conjugation and is C11-OH linked;
(ii) Ab-Gly-Glu-PAB-DMEDA-INX-3 or Ab-GlcA-PAB-DMEDA-INX-SM-3 (azide+dibenzocyclooctyne conjugation (C11-OH linked), or another ADC comprising a different INX-SM payload wherein the INX-SM payload is conjugated to the antibody via azide+dibenzocyclooctyne conjugation and is C11-OH linked;
(iii) Ab-Gly-Glu-PAB-DMEDA-INX-3 or Ab-GlcA-PAB-DMEDA-INX-SM-3 (haloacetyl+cysteine conjugation (C11-OH linked), or another ADC comprising a different INX-SM payload wherein the INX-SM payload is conjugated to the antibody via azide+dibenzocyclooctyne conjugation and is C11-OH linked;
(iv) Ab-Gly-Glu-PAB-DMEDA-INX-3 or Ab-GlcA-PAB-DMEDA-INX-SM-3 (maleimide+cysteine conjugation (C11-OH linked), or another ADC comprising a different INX-SM payload wherein the INX-SM payload is conjugated to the antibody via azide+dibenzocyclooctyne conjugation and is C11-OH linked; Ab-Gly-Glu-PAB-DMEDA-INX-3 or Ab-GlcA-PAB-DMEDA-INX-SM-3 (tetrazine (v)+trans-cyclooctene conjugation (C11-OH linked), or another ADC comprising a different INX-SM payload wherein the INX-SM payload is conjugated to the antibody via tetrazine+trans-cyclooctene conjugation and is C11-OH linked; (vi) Ab-Gly-Glu-PAB-DMEDA-INX-3 or Ab-GlcA-PAB-DMEDA-INX-SM-3 (Alkoxyamine+Ketone conjugation (C11-OH linked)), or another ADC comprising a different INX-SM payload wherein the INX-SM payload is conjugated to the antibody via Alkoxyamine+Ketone conjugation and is C11-OH linked;
(vii) Ab-Gly-Glu-PAB-DMEDA-INX-3 or Ab-GlcA-PAB-DMEDA-INX-SM-3 (Azide+Dibenzocyclooctyne conjugation (C17 linked)), or another ADC comprising a different INX-SM payload wherein the INX-SM payload is conjugated to the antibody via Azide+Dibenzocyclooctyne Ketone conjugation and is C17 linked; (viii) Ab-Gly-Glu-PAB-DMEDA-INX-3 or Ab-GlcA-PAB-DMEDA-INX-SM-3 (Haloacetyl+Cysteine conjugation (C17 linked)), or another ADC comprising a different INX-SM payload wherein the INX-SM payload is conjugated to the antibody via Azide+Dibenzocyclooctyne Ketone conjugation and is C17 linked;
(ix) Ab-Gly-Glu-PAB-DMEDA-INX-3 or Ab-GlcA-PAB-DMEDA-INX-SM-3 (Tetrazine+Trans-cyclooctene conjugation (C17 linked)), or another ADC comprising a different INX-SM payload wherein the INX-SM linker payload is conjugated to the antibody via Tetrazine+Trans-cyclooctene conjugation and is C17 linked;
(x) Ab-Gly-Glu-PAB-DMEDA-INX-3 or Ab-GlcA-PAB-DMEDA-INX-SM-3 (Amine+Glutamine conjugation using trans glutaminase (C17 linked)), or another ADC comprising a different INX-SM payload wherein the INX-SM linker payload is conjugated to the antibody via Amine+Glutamine conjugation using trans glutaminase and is C17 linked;
(xi) INX-SM-3-PAB-GlcA-Ab or INX-SM-3-PAB-Glu-Gly-Ab (alkoxyamine and Ketone Conjugation) (N-linked payload) or another ADC comprising a different INX-SM payload wherein the INX-SM linker payload is conjugated to the antibody via alkoxyamine and Ketone Conjugation and is N linked;
(xii) INX-SM-3-PAB-GlcA-Ab or INX-SM-3-PAB-Glu-Gly-Ab (haloacetyl Conjugation) (N-linked payload) or another ADC comprising a different INX-SM payload wherein the INX-SM linker payload is conjugated to the antibody via haloacetyl Conjugation and is N linked;
(xiii) INX-SM-3-PAB-GlcA-Ab or INX-SM-3-PAB-Glu-Gly-Ab (Azide+Dibenzocyclooctyne Conjugation) (N-linked payload) or another ADC comprising a different INX-SM payload wherein the INX-SM linker payload is conjugated to the antibody Azide+Dibenzocyclooctyne Conjugation and is N linked;
(xiv) INX-SM-3-GlcA-Ab or INX-SM-3-Glu-Gly-Ab (N-hydroxysuccinimide Conjugation) (N-linked payload) or another ADC comprising a different INX-SM payload wherein the INX-SM linker payload is conjugated to the antibody via N-hydroxysuccinimide Conjugation and is N linked;
(xv) INX-SM-3-PAB-GlcA-Ab or INX-SM-3-PAB-Glu-Gly-Ab (Azide+Dibenzocyclooctyne Conjugation) (N-linked payload) or another ADC comprising a different INX-SM payload wherein the INX-SM linker payload is conjugated to the antibody via Azide+Dibenzocyclooctyne Conjugation and is N linked;
(xvi) INX-SM-3-PAB-GlcA-Ab or INX-SM-3-PAB-Glu-Gly-Ab (N-hydroxysuccinimide Conjugation) (N-linked payload) or another ADC comprising a different INX-SM payload wherein the INX-SM linker payload is conjugated to the antibody via N-hydroxysuccinimide Conjugation and is N linked;
(xvii) INX-SM-3-Glu-Gly-Ab or INX-SM-3-PAB-Glu-Gly-Ab (Maleimide Conjugation) (N-linked payload) or another ADC comprising a different INX-SM payload wherein the INX-SM linker payload is conjugated to the antibody via Maleimide Conjugation and is N linked;
(xviii) INX-SM-3-Glu-Gly-Ab or INX-SM-3-PAB-Glu-Gly-Ab or INX-SM-3-PAB-GlcA-Ab (Trans-cyclooctene+Tetrazine Conjugation) (N-linked payload) or another ADC comprising a different INX-SM payload wherein the INX-SM linker payload is conjugated to the antibody via Trans-cyclooctene+Tetrazine Conjugation and is N linked;
(xix) INX-SM-3-Glu-Gly-Ab or INX-SM-3-PAB-Glu-Gly-Ab or INX-SM-3-PAB-GlcA-Ab (Amine Conjugation) (N-linked payload) or another ADC comprising a different INX-SM payload wherein the INX-SM linker payload is conjugated to the antibody via Trans-cyclooctene+Tetrazine Conjugation and is N linked.

It is a another specific object of the invention to provide an antibody drug conjugate (ADC) selected from the following:

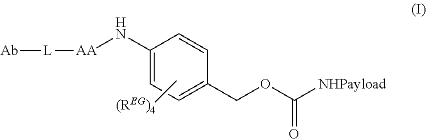

wherein,
Ab=Antibody, preferably an antibody that binds to human immune cells, preferably an anti-VISTA antibody that binds to human VISTA immune cells at physiologic pH;
L=Linker;
AA=Single, double, or triple amino acid sequence;

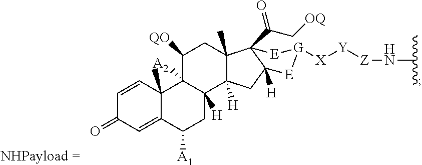

REG is independently selected from the group consisting of hydrogen, alkyl, biphenyl, —CF3, —NO2, —CN, fluoro, bromo, chloro, alkoxyl, alkylamino, dialkylamino, alkyl-C(O)O—, alkylamino-C(O)— and dialkylaminoC(O)—;

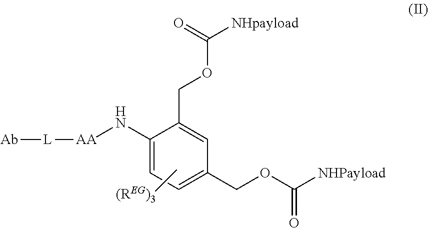

Ab=Antibody;
L=Linker;
AA=Single, double, or triple amino acid sequence;

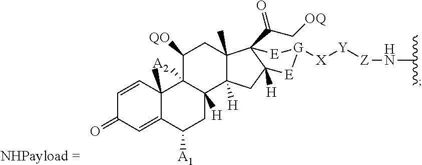

REG is independently selected from the group consisting of hydrogen, alkyl, biphenyl, —CF3, —NO2, —CN, fluoro, bromo, chloro, alkoxyl, alkylamino, dialkylamino, alkyl-C(O)O—, alkylamino-C(O)— and dialkylaminoC(O)—;

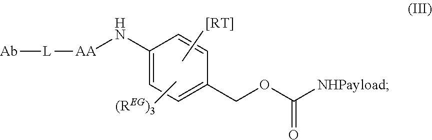

Ab=Antibody;
L=Linker;
AA=Single, double, or triple amino acid sequence or not present;

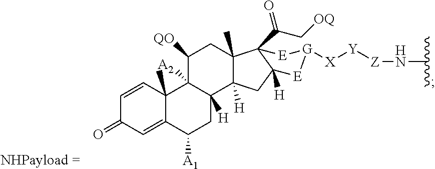

REG is independently selected from the group consisting of hydrogen, alkyl, biphenyl, —CF3, —NO2, —CN, fluoro, bromo, chloro, alkoxyl, alkylamino, dialkylamino, alkyl-C(O)O—, alkylamino-C(O)— and dialkylaminoC(O)—;

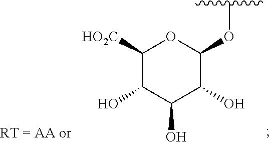 [SMILES: *O[C@@H]1O[C@H](C(=O)O)[C@@H](O)[C@H](O)[C@H]1O]

Ab=Antibody, optionally an anti-human VISTA antibody;
L=Linker,
AA=Single, double, or triple amino acid sequence;

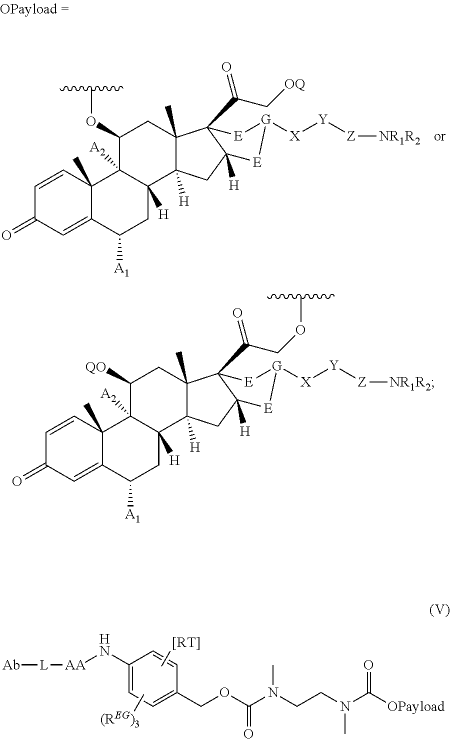

Ab=Antibody, typically one which binds to an antigen expressed on immune cells, typically human immune cells, e.g., VISTA;
L=Linker;
AA=Single, double, or triple amino acid sequence or not present;

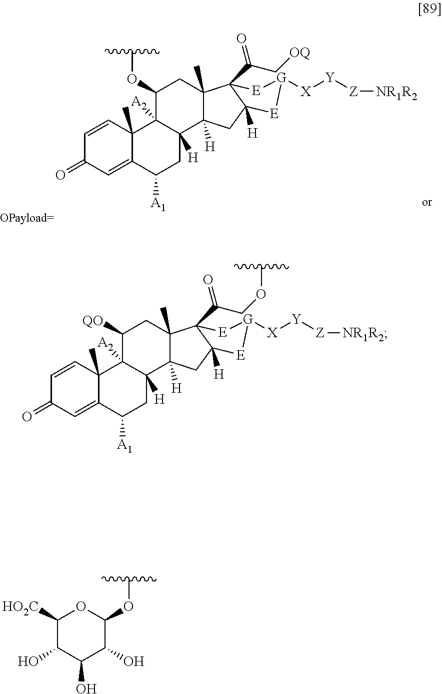

It is a specific object of the invention to provide an antibody drug conjugate (ADC) according to the foregoing, wherein the linker comprises a cleavable or non-cleavable peptide or immolative linker.

It is a specific object of the invention to provide an antibody drug conjugate (ADC) according to any of the foregoing, which comprises a linker which is selected from PAB and/or an amino acid or a peptide, optionally 1-12 amino acids, further optionally dipeptide, a tripeptide, a quatrapeptide, a pentapeptide and further optionally Gly, Asn, Asp, Gln, Leu, Lys, Ala, Phe, Cit, Val, Val-Cit, Val-Ala, Val-Gly, Val-Gln, Ala-Val, Cit-Cit, Lys-Val-Cit, Asp-Val-Ala, Ala-Ala-Asn, Asp-Val-Ala, Ala-Val-Cit, Ala-Asn-Val, betaAla-Leu-Ala-Leu, Lys-Val-Ala, Val-Leu-Lys, Asp-Val-Cit, Val-Ala-Val, and Ala-Ala-Asn.

It is a specific object of the invention to provide a steroid antibody conjugate compound selected from the following structures:

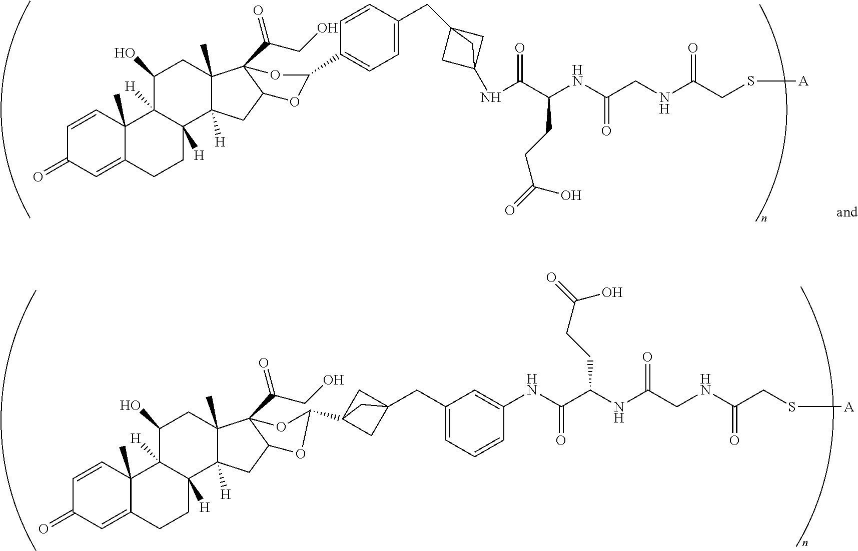

where n=2-12, 2-10, 2-8, 2-6, 2-4 and A is an antibody or antigen binding fragment thereof, preferably an antibody or antibody fragment which binds to an antigen expressed on an immune cell, preferably a human immune cell, and in exemplary embodiments an anti-human VISTA antibody.

It is a specific object of the invention to provide a glucocorticoid agonist compound of Formula (I):

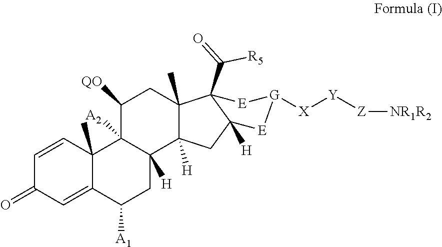

wherein
X is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo[2.2.2]octane, adamantane, and cubane each of which can be substituted with 1-4 heteroatoms independently selected from F, Cl, Br, I, N, S, and O, each of which ring structure may contain at least one skeletal heteroatom selected from N, S, and O, and are optionally further substituted with 1-4 C1-3 alkyl or C1-3 perfluoroalkyl;
Z is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo[2.2.2]octane, adamantane, and cubane each of which can be substituted with 1-4 heteroatoms independently selected from F, Cl, Br, I, N, S, and O, each of which ring structure may contain at least one skeletal heteroatom selected from N, S, and O, and are optionally further substituted with 1-4 C1-3 alkyl or C1-3 perfluoroalkyl;
Y is selected from CHR1, O, S, and NR1;
E is selected from CH2 and O;
G is selected from CH, and N;
further wherein when G is CH and X is phenyl, Z is not phenyl;
the linkage of G to X may optionally be selected from C1-3 alkyl and ethylene oxide, each of which may be substituted with 1-4 heteroatoms independently selected from N, S, and O and are optionally further substituted with 1-4 C1-3 alkyl;
the linkage of X to Z may occupy any available position on X and Z;
substituent NR1R2 may occupy any available position on Z;
R1 is selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH2, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylaminoC(O)—;
when R1 is H, R2 may be selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH2, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylaminoC(O)—;
when R1 is H, linear or branched alkyl of 1-8 carbons, or heteroaryl, R2 may be a functional group selected from

[(C═O)CH(W)NH]m-[C═O]—[V]k-J,

(C═O)OCH2-p-aminophenyl-N—V-J,

(C═O)OCH2-p-aminophenyl-N—[(C═O)CH(W)NH]m-[C═O]—[V]k-J, and

[V]k-(C═O)OCH2-p-aminophenyl-N—[(C═O)CH(W)NH]m-[C═O]-J,
wherein m=1-6, k=0-1, and each permutation of W may independently be selected from H, [(CH2)nR3] where n=1-4, a branched alkyl chain terminating in R3, and a linear or branched polyethylene oxide group comprising 1-13 units;
R3 is selected from H, methyl, ethyl, isopropyl, OH, O-alkyl, NH2, NH-alkyl, N-dialkyl, SH, S-alkyl, guanidine, urea, carboxylic acid, carboxamide, carboxylic ester, substituted or unsubstituted aryl, substituted or unsubstituted heteroaryl, wherein said aryl and heteroaryl substituents may be selected from alkyl, haloalkyl, halogen, biphenyl, nitro, nitrile, —OH, —O-alkyl, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, and dialkylaminoC(O)—;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—;
alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH2, N3, thio, cyclooctyne, —OH, —CO2H, trans-cyclooctene, alkynyl, propargyl,

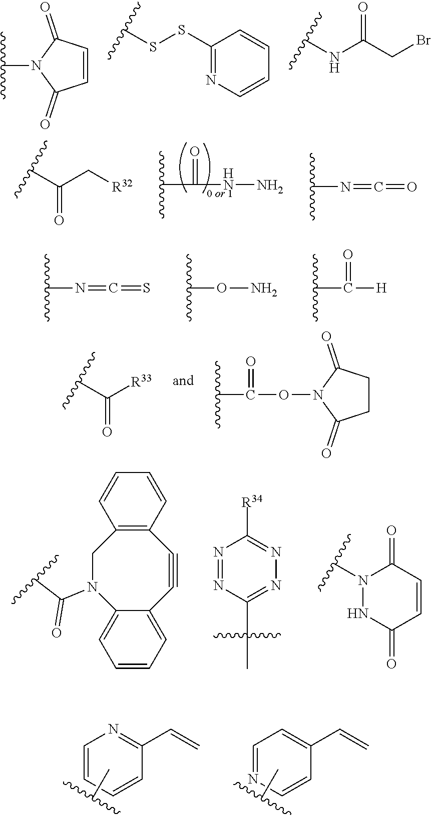

and where R32 is selected from Cl, Br, F, mesylate, and tosylate and R33 is selected from Cl, Br, I, F, OH, —O—N-succinimidyl, —O-(4-nitrophenyl), —O-pentafluorophenyl or —O-tetrafluorophenyl R34 is H, Me, tetrazine-H, and tetrazine-Me;
R5 is selected from the group consisting of —CH₂OH, —CH2SH, —CH2Cl, —SCH2Cl, —SCH2F, —SCH2CF3, hydroxy, —OCH2CN, —OCH2Cl, —OCH2F, —OCH3, —OCH2CH3, —SCH2CN, and

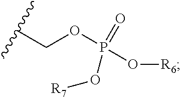

R6 and R7 are independently selected from hydrogen and C1-10 alkyl;

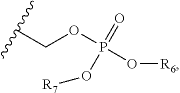

Q may be H, C(O)R8 where R8 is linear or branched alkyl of 1-8 carbons, or (C═O)NR4CHnNR4(C═O)OCH2-(V)n-J where n=1-4 and R4=H, alkyl or branched alkyl, or P(O)OR4;
A1 and A2 are independently selected from H and F; and
unless otherwise specified, all possible stereoisomers are included.

It is a specific object of the invention to provide a glucocorticoid agonist compound according to any of the foregoing, wherein Z is selected from

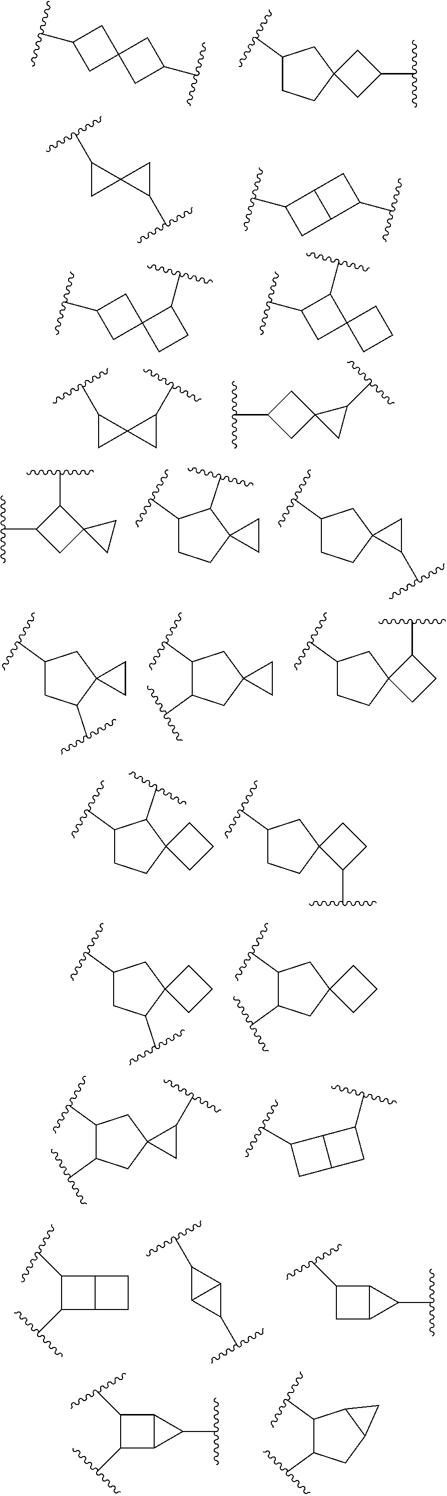

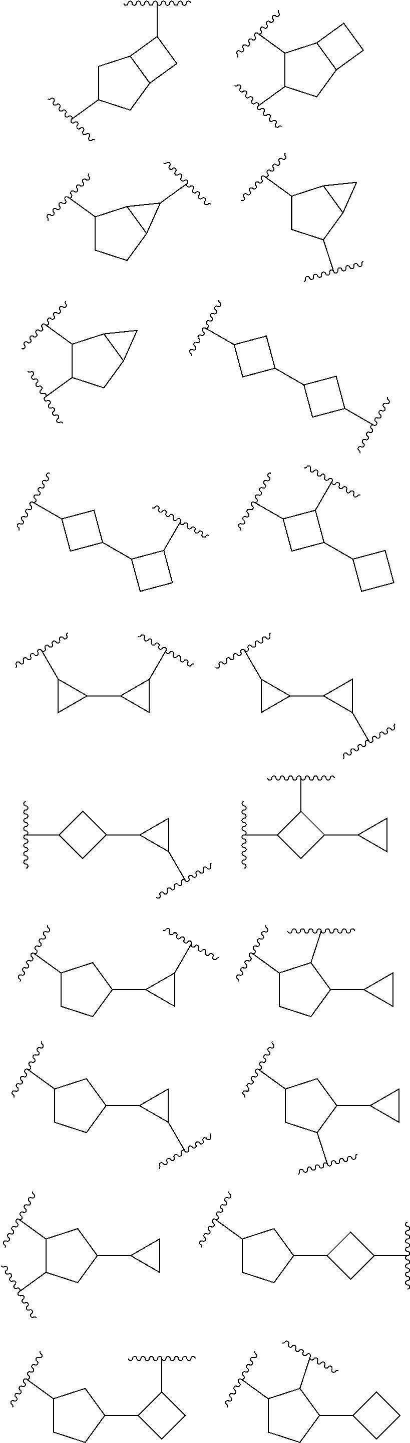

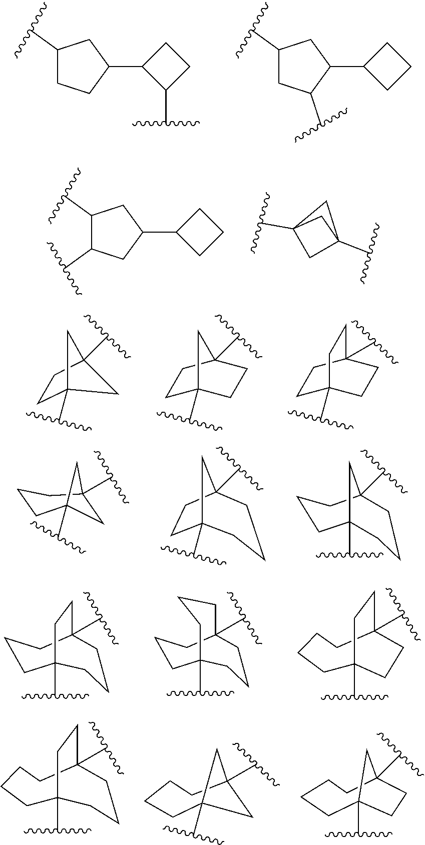

each of which may be substituted with 1-4 heteroatoms independently selected from F, Cl, Br, I, N, S, and O, and are optionally further substituted with 1-4 C1-3 alkyl or C1-3 perfluoroalkyl groups;
each of which ring structure may contain at least one additional skeletal heteroatom selected from N, S, and O; and
wherein each

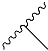

indicates a point of attachment to the rest of the formula and each of said points of attachment may be covalently bonded to the rest of the formula via an additional heteroatom selected from N, S, and O.

It is a specific object of the invention to provide a glucocorticoid agonist compound according to any of the foregoing, wherein Z—NR1 is selected from

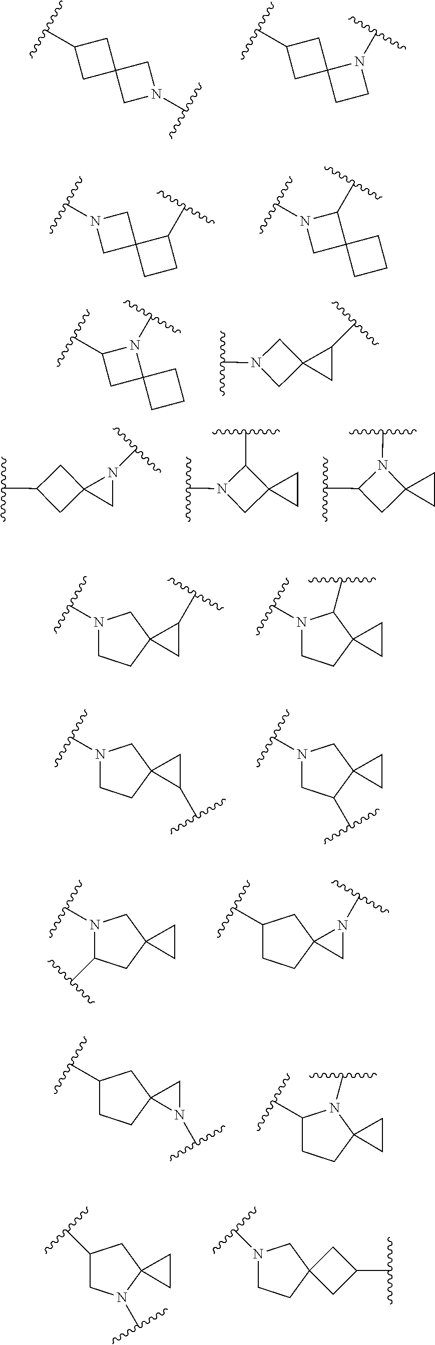

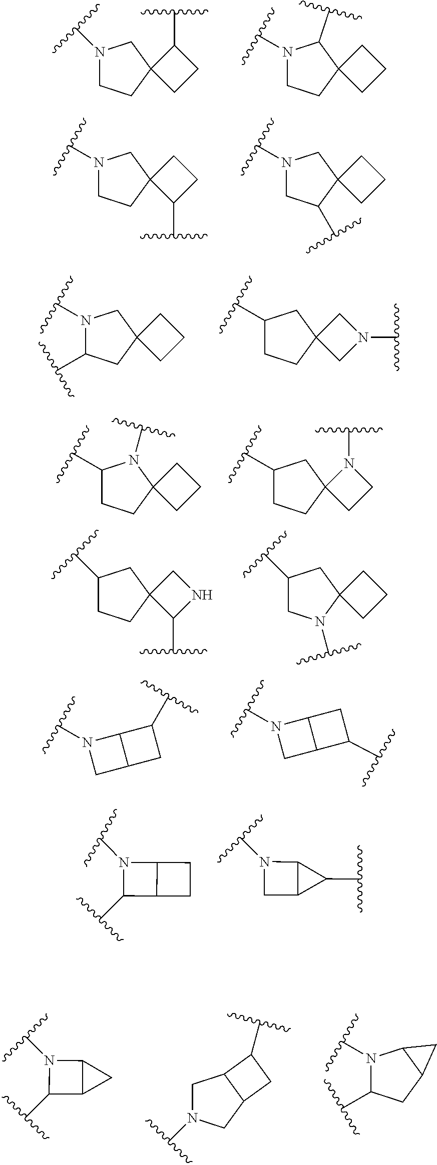

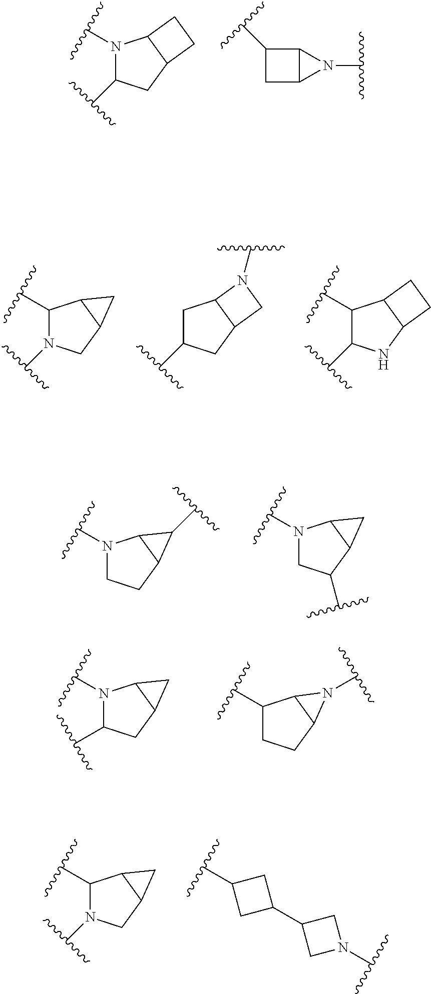

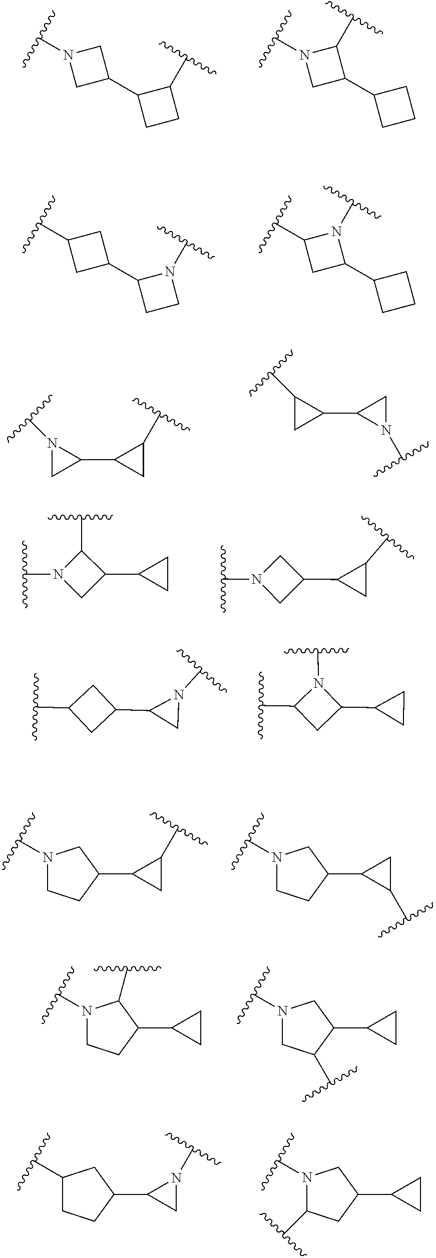

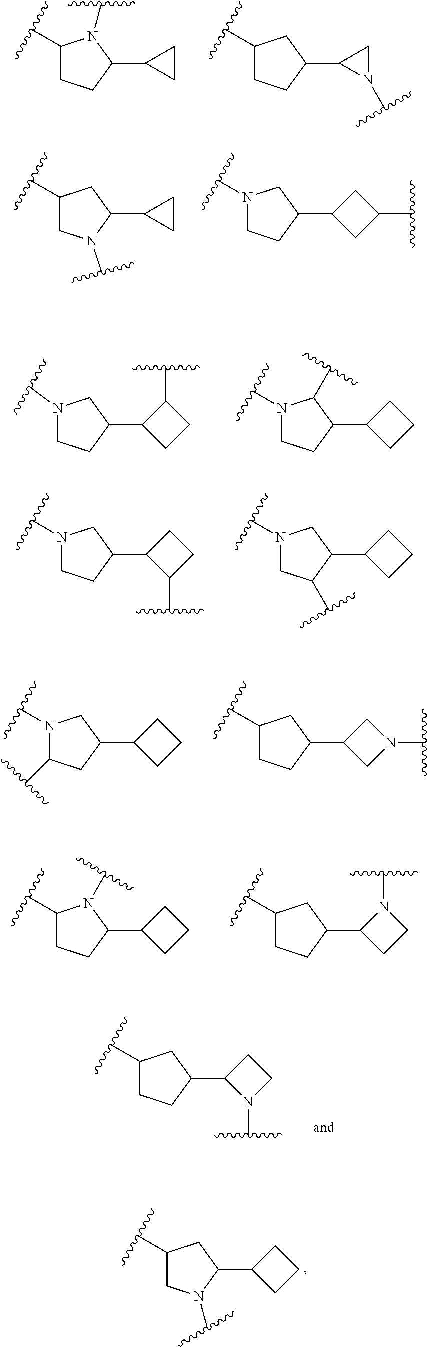

each of which may be substituted with 1-4 heteroatoms independently selected from F, Cl, Br, I, N, S, and O, and are optionally further substituted with 1 to 4 C1-3 alkyl or C1-3 perfluoroalkyl groups;
each of which ring structure may contain at least one additional skeletal heteroatom selected from N, S, and O; and
wherein each indicates

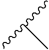

a point of attachment to the rest of the formula and each of said points of attachment may be covalently bonded to the rest of the formula via an additional heteroatom selected from N, S, and O.

It is a specific object of the invention to provide a glucocorticoid agonist compound which possesses the structure of Formula (II):

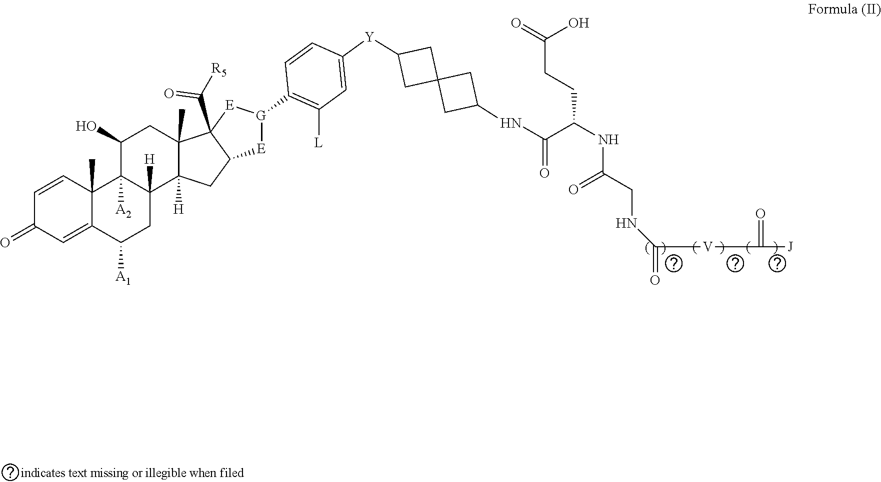

wherein
Y is selected from CH₂ and O;
E is selected from CH₂ and O;
G is selected from CH, and N;
L is selected from H and F;
R₅ is selected from —CH₂OH, —SCH₂F and

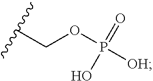 [SMILES: *COP(=O)(O)O]

A₁ and A₂ are independently selected from H and F;

V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH2, N3, thio, cyclooctyne, —OH, —CO2H, trans-cyclooctene, alkynyl, propargyl,

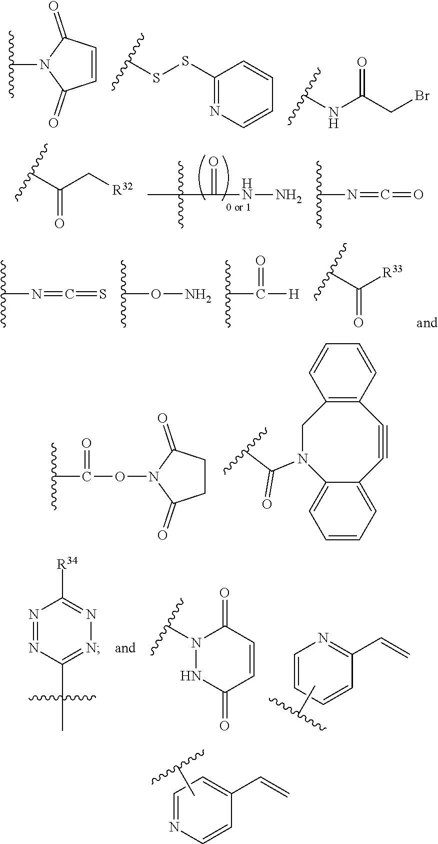

where R32 is selected from Cl, Br, F, mesylate, and tosylate and R33 is selected from Cl, Br, I, F, OH, —O—N-succinimidyl, —O-(4-nitrophenyl), —O-pentafluorophenyl or —O-tetrafluorophenyl R34 is H, Me, tetrazine-H, and tetrazine-Me.

It is a specific object of the invention to provide a glucocorticoid agonist compound according to any of the foregoing, which possesses the structure of Formula (III):

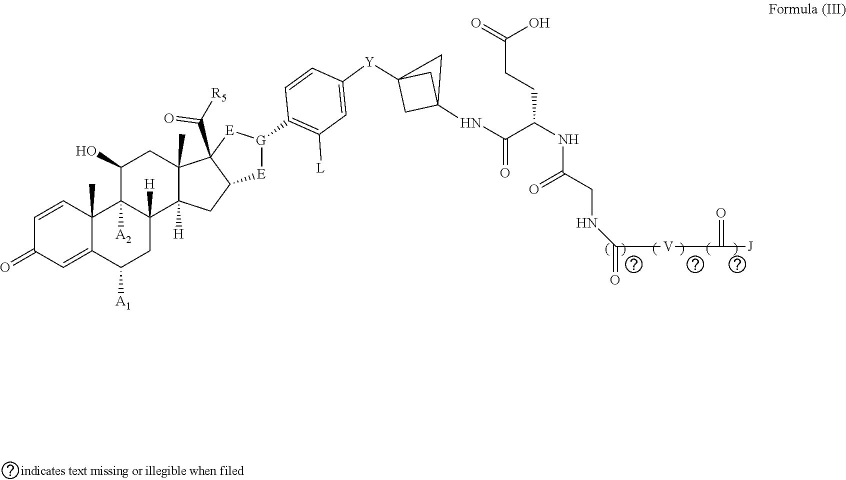

wherein
Y is selected from CH2 and O;
E is selected from CH2 and O;
G is selected from CH, and N;
L is selected from H and F;
R5 is selected from —CH₂OH, —SCH2F and

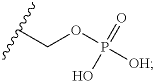 [SMILES: *COP(=O)(O)O]

A1 and A2 are independently selected from H and F;
V may be selected from an alkyl chain of 1-8 carbons; a linear or branched polyethylene oxide group comprising 1-13 units; linear or branched alkyl group comprising 1-8 carbons; —O-alkyl; carboxylic carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH2, N3, thio, cyclooctyne, —OH, —CO2H, trans-cyclooctene, alkynyl, propargyl,

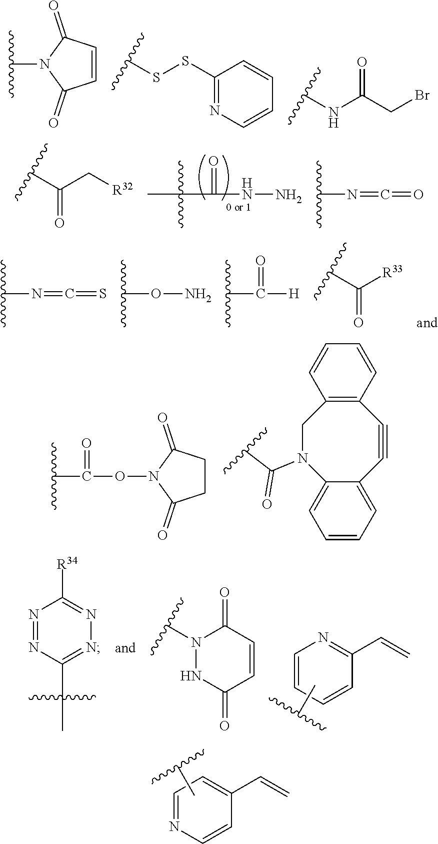

where R32 is selected from Cl, Br, F, mesylate, and tosylate and R33 is selected from Cl, Br, I, F, OH, —O—N-succinimidyl, —O-(4-nitrophenyl), —O-pentafluorophenyl or —O-tetrafluorophenyl R34 is H, Me, tetrazine-H, and tetrazine-Me.

It is a specific object of the invention to provide a glucocorticoid agonist compound according to any of the foregoing, which is selected from:

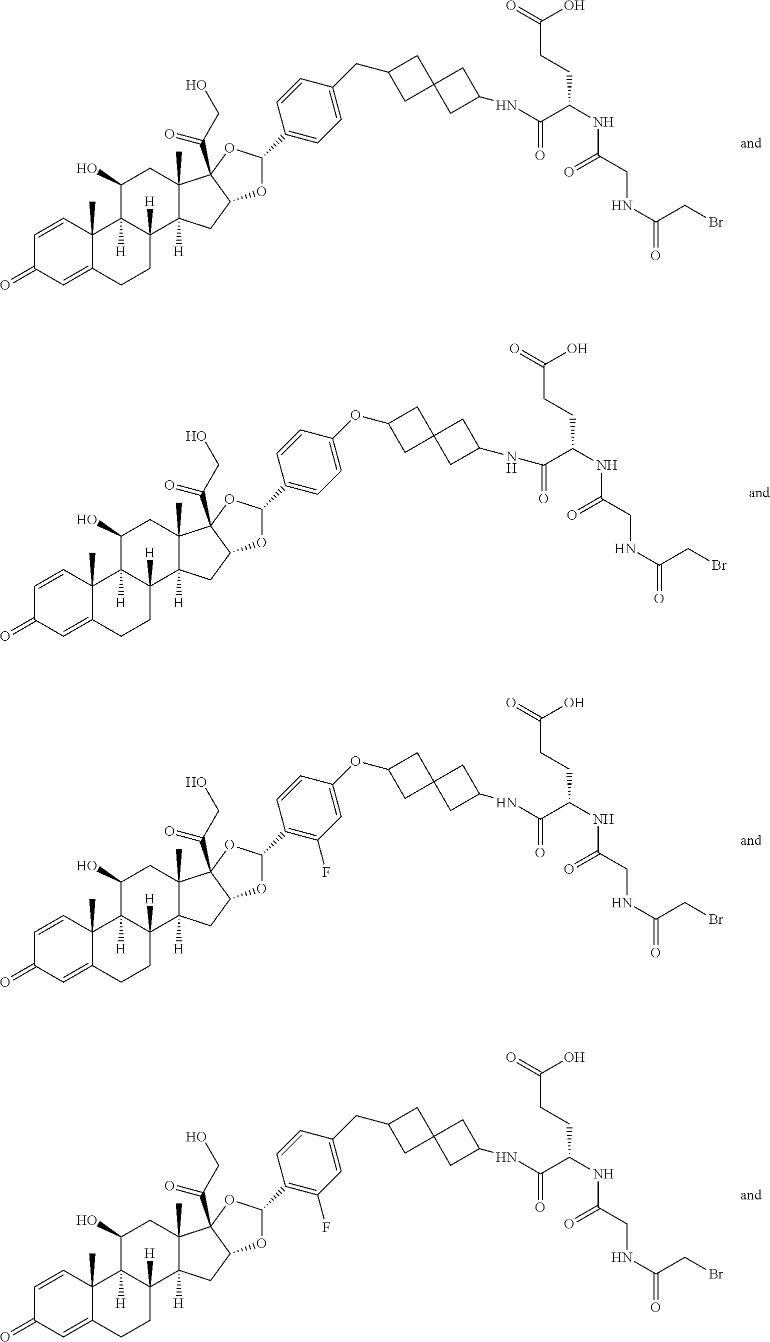

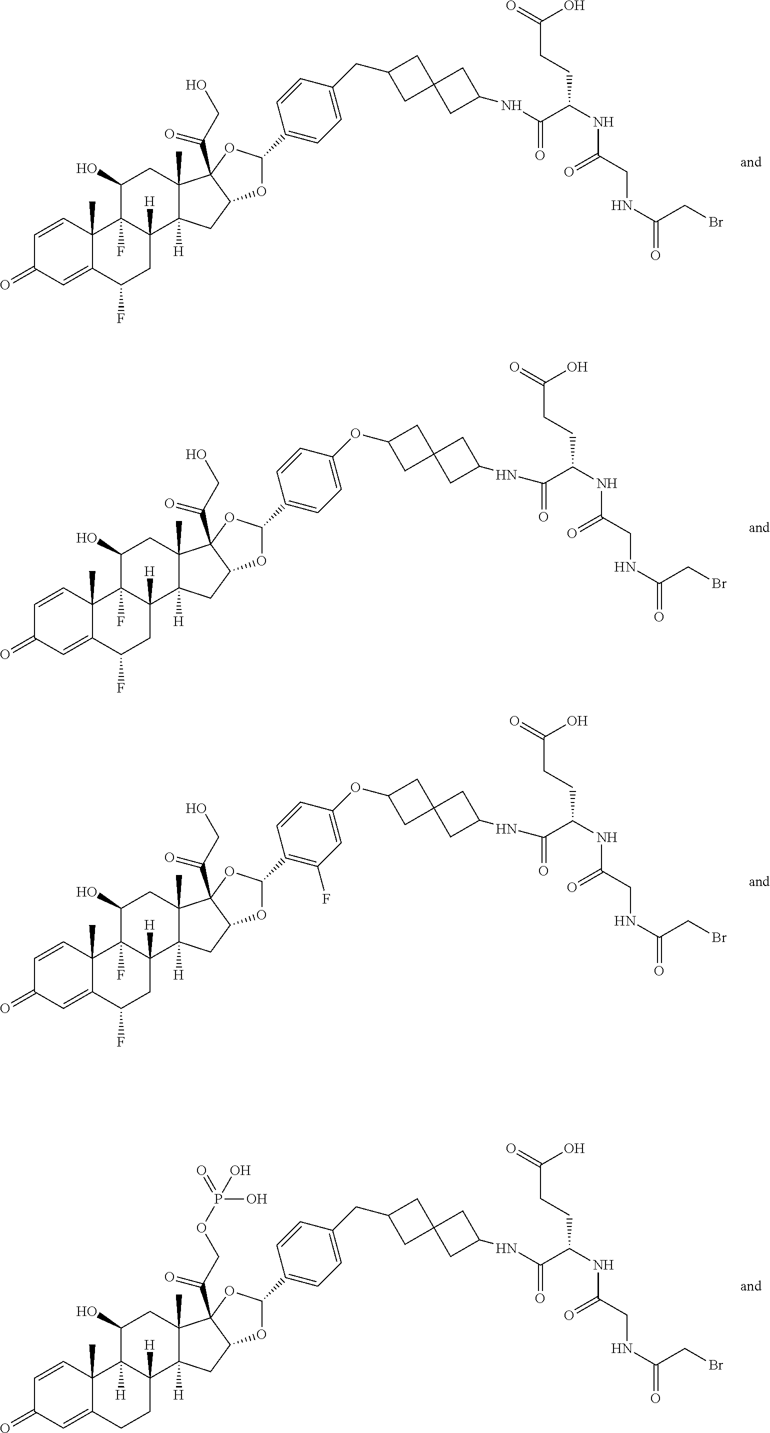

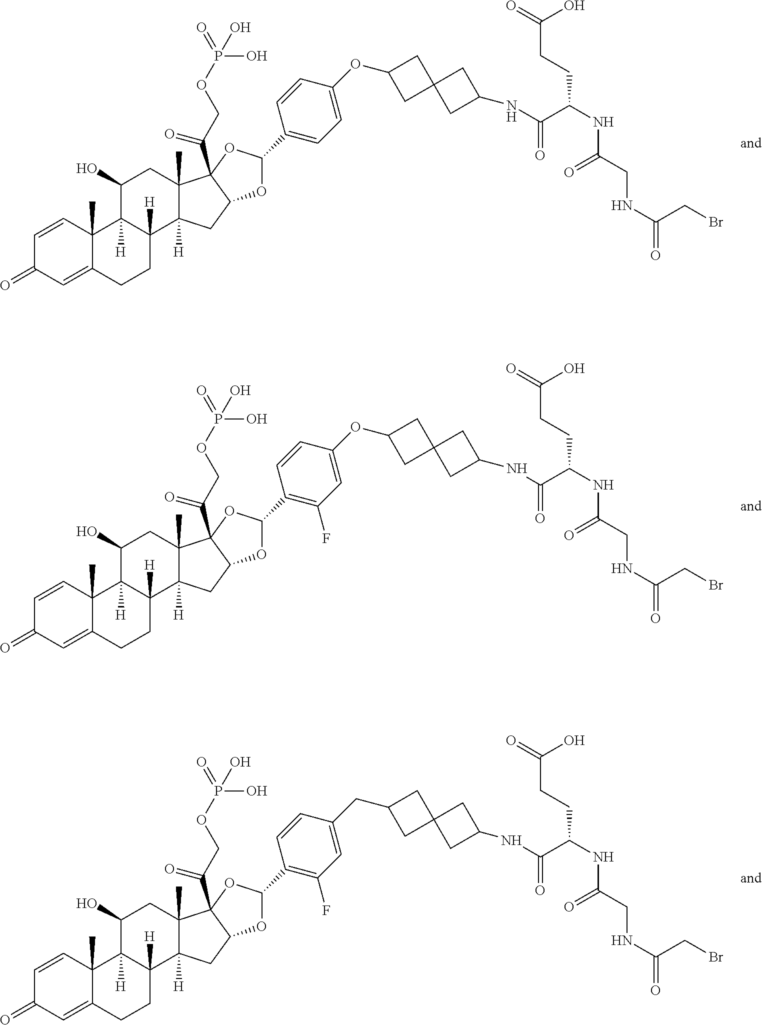

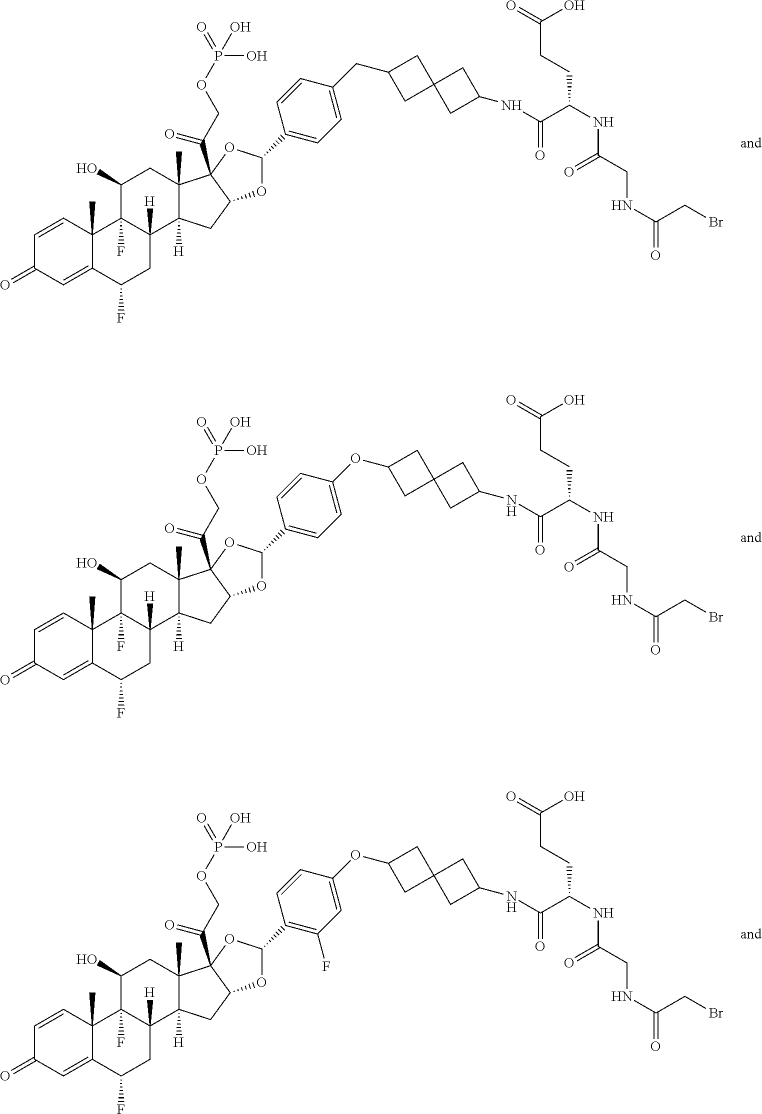

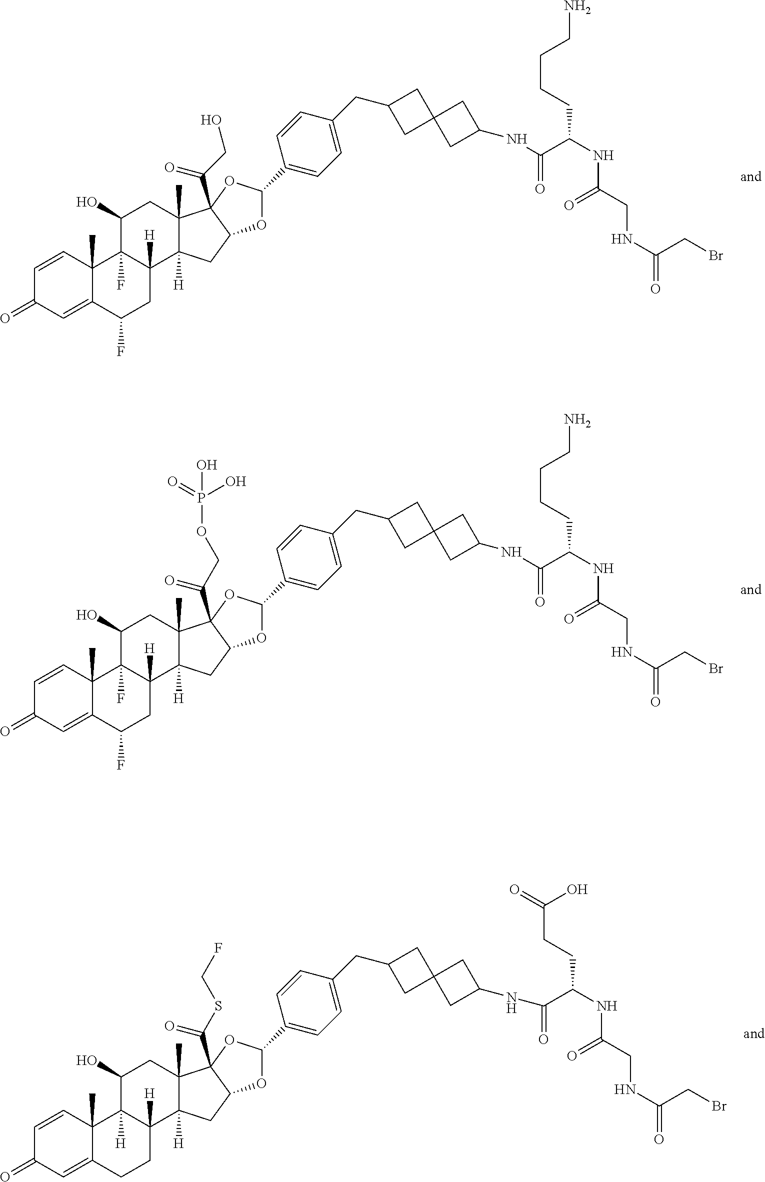

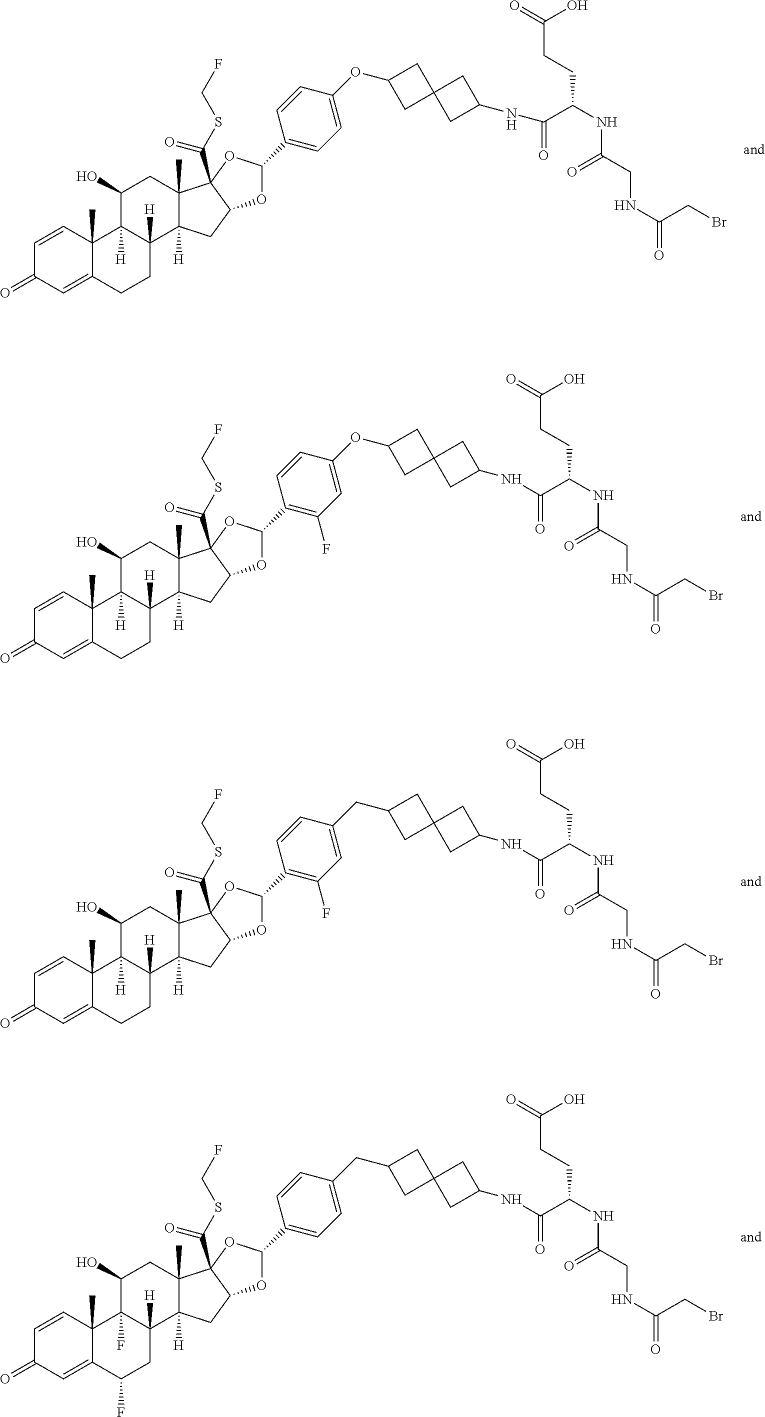

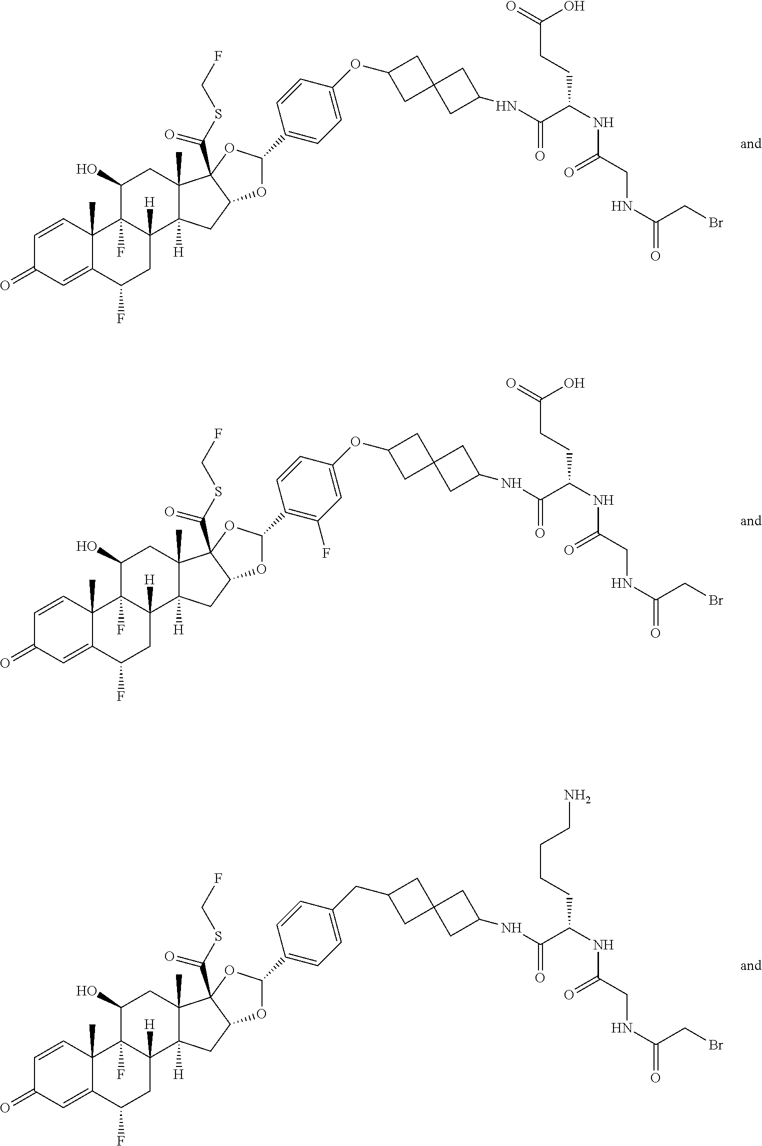

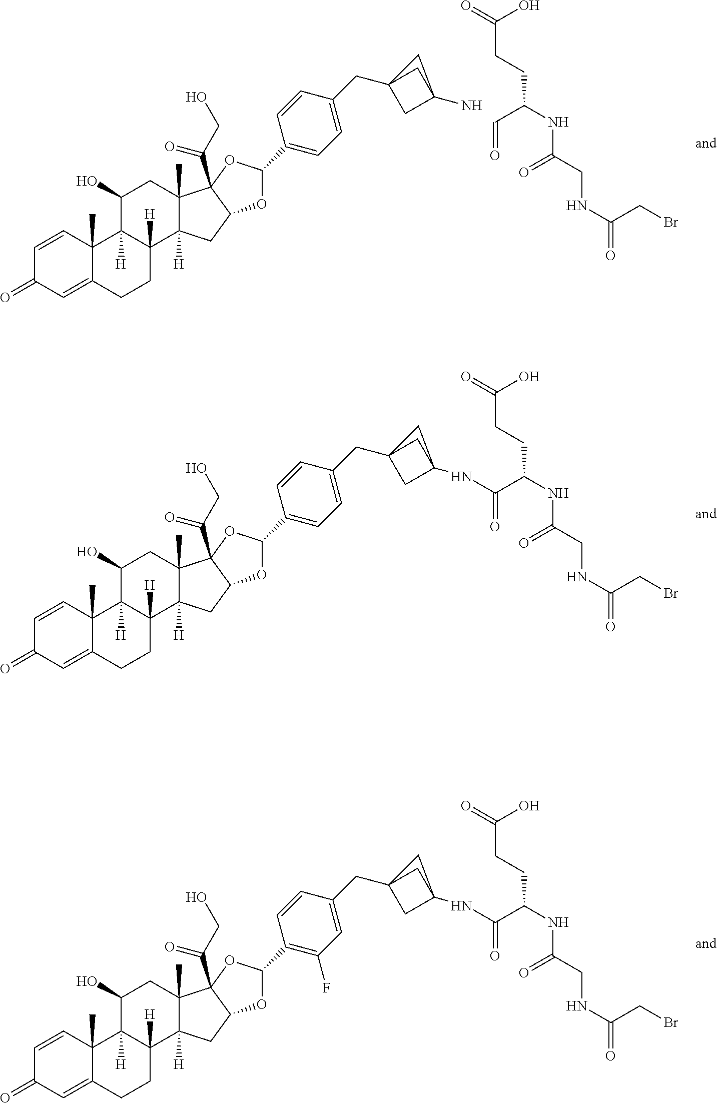

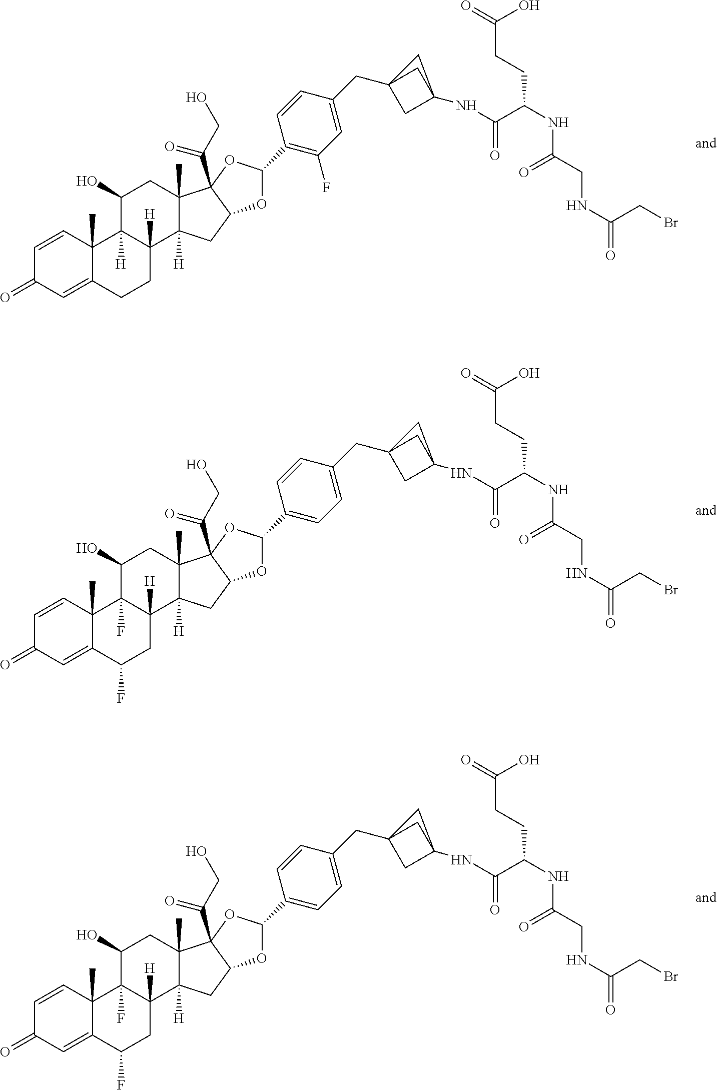

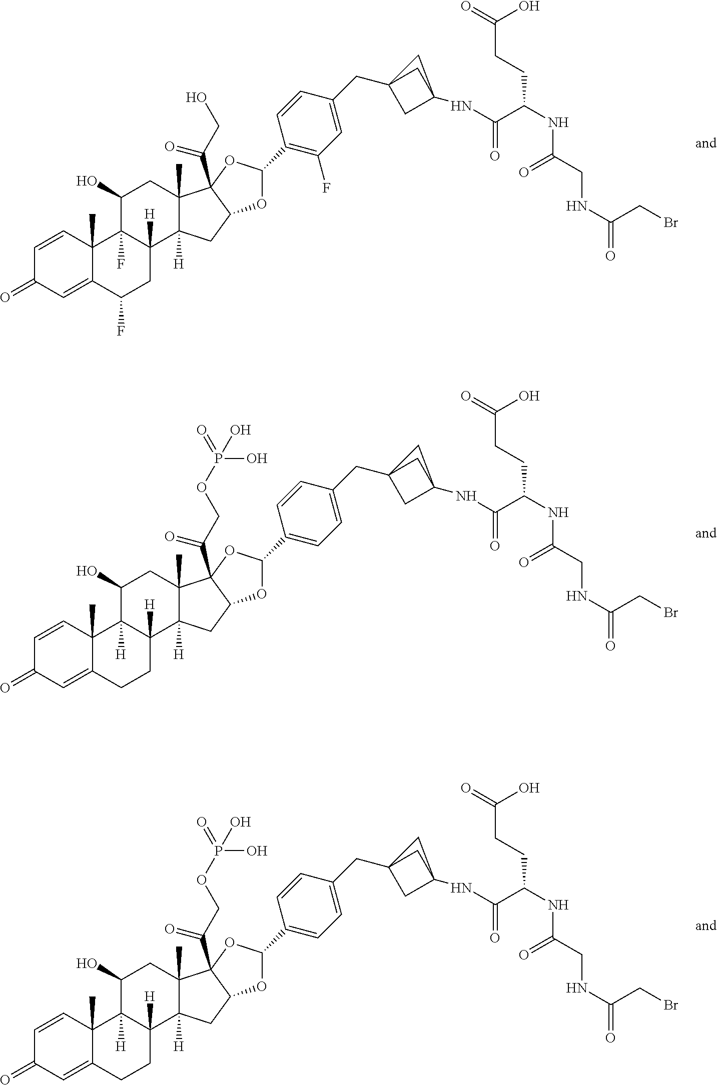

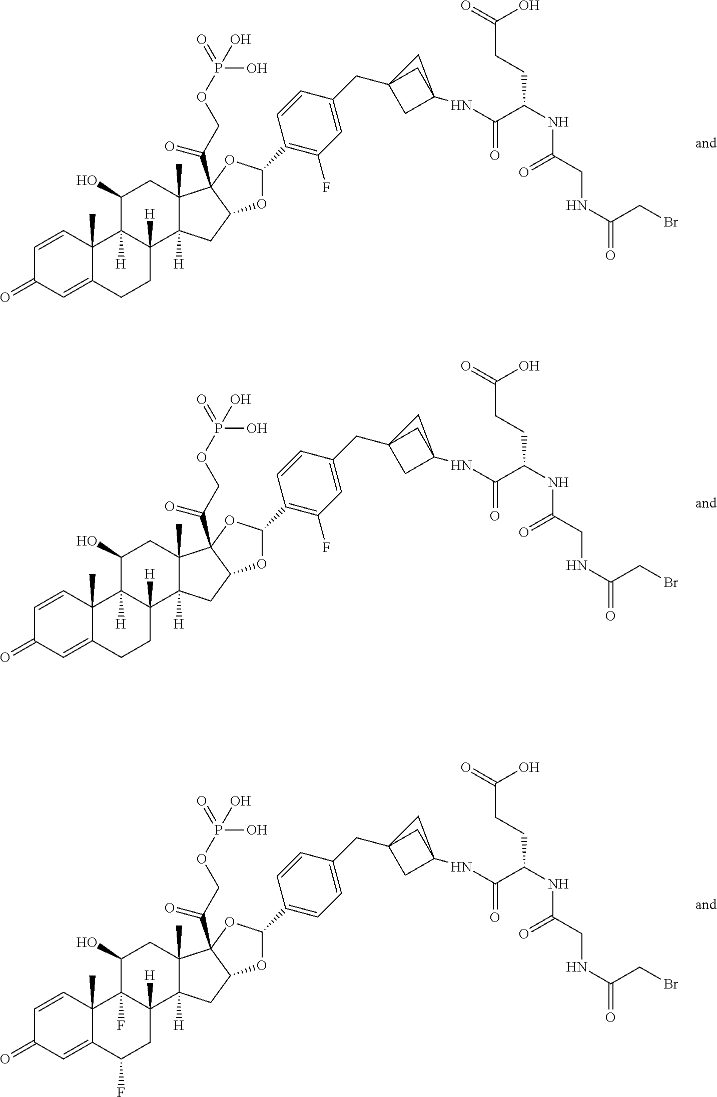

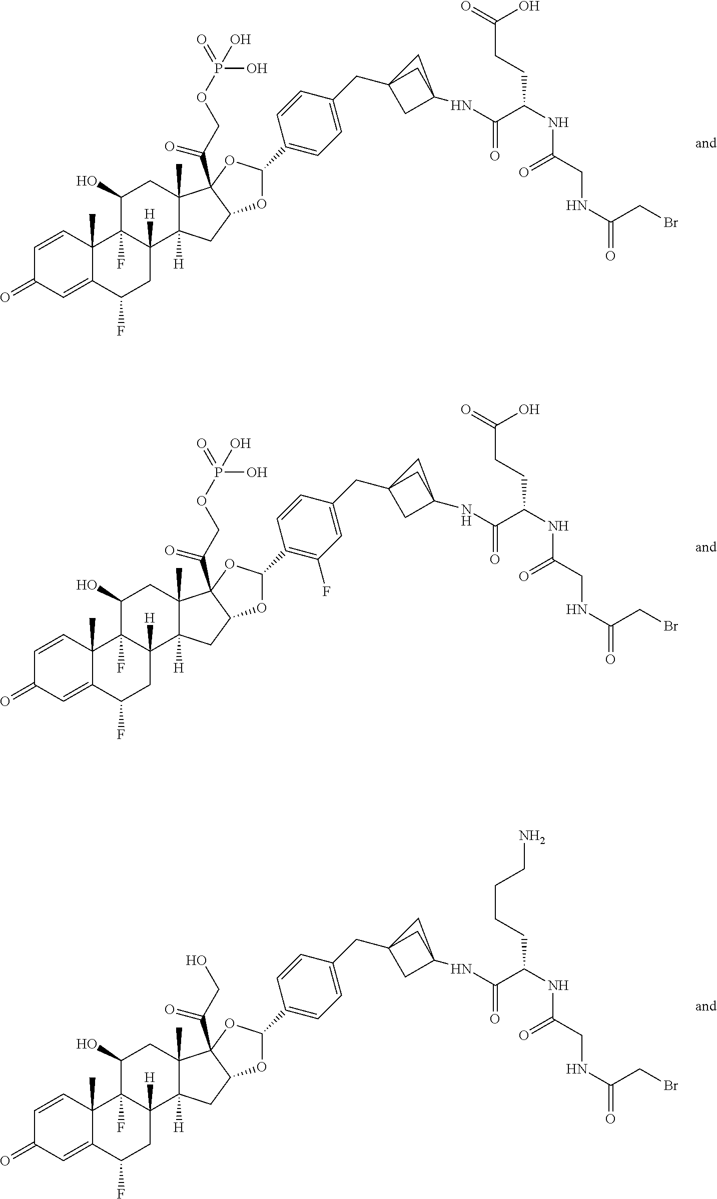

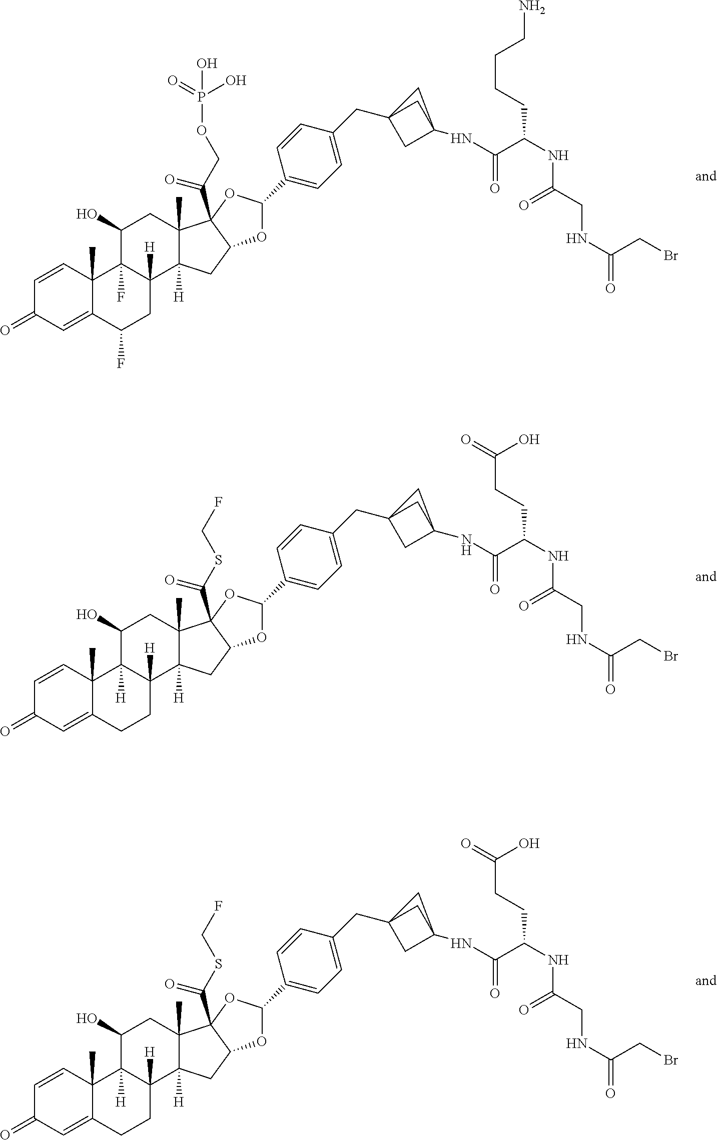

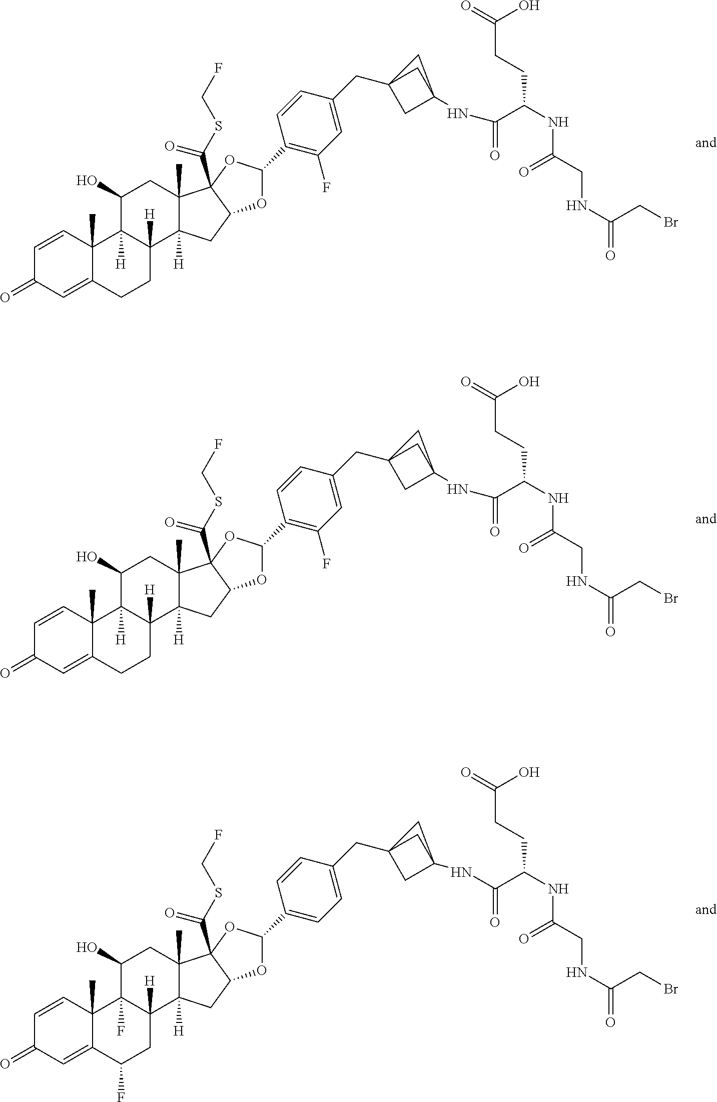

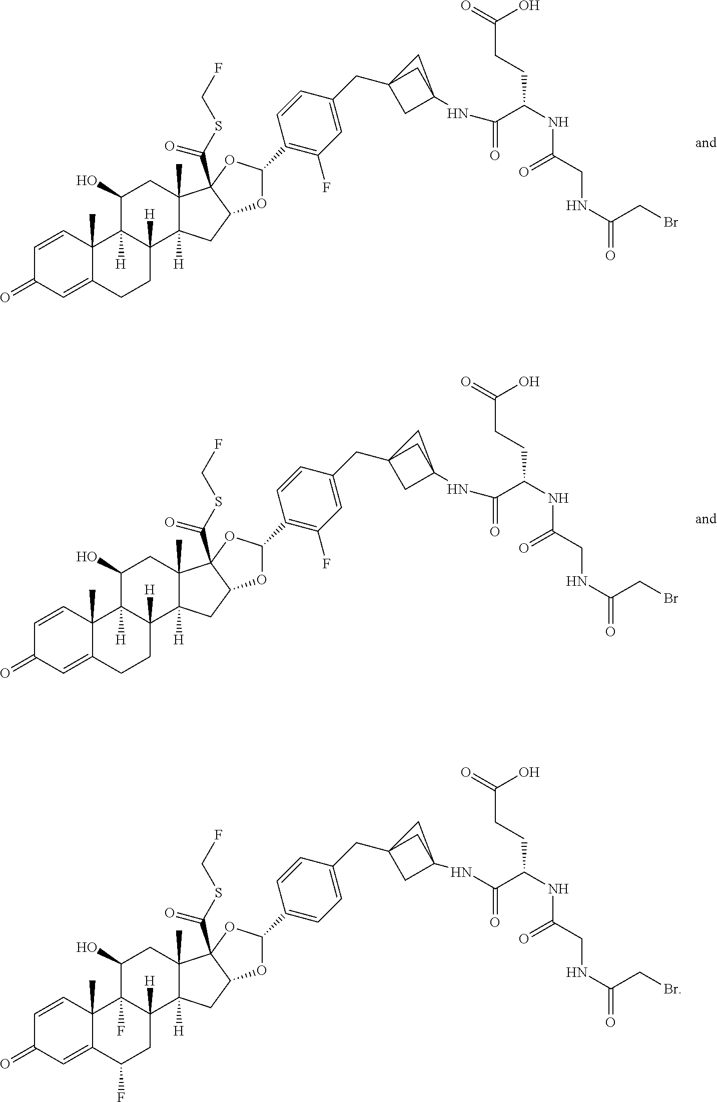

It is a specific object of the invention to provide a glucocorticoid agonist compound according to any of the foregoing, i.e., of Formula I, II or III, wherein X or Z may be spiro[3.3]heptane or [1.1.1]bicyclopentane and Y may be CH2 or O.

It is a specific object of the invention to provide an antibody drug conjugate (ADC) which comprises an antibody or antigen binding fragment thereof, preferably one which binds to an antigen expressed by an immune cell, preferably a human immune cell, which antibody or antigen binding fragment thereof, is attached to at least one glucocorticoid agonist compound according to any of the previous embodiments.

It is a specific object of the invention to provide an ADC which is selected from:

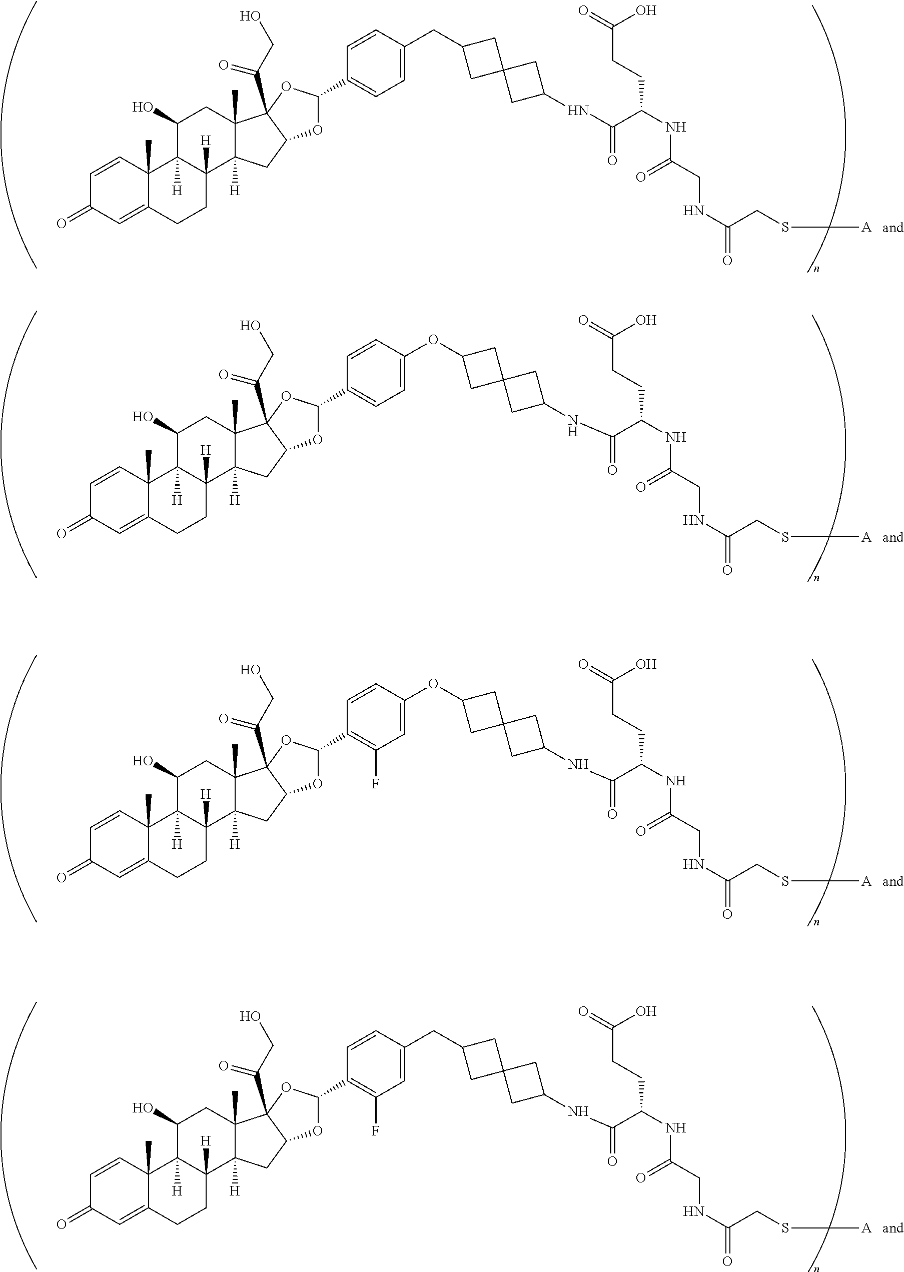

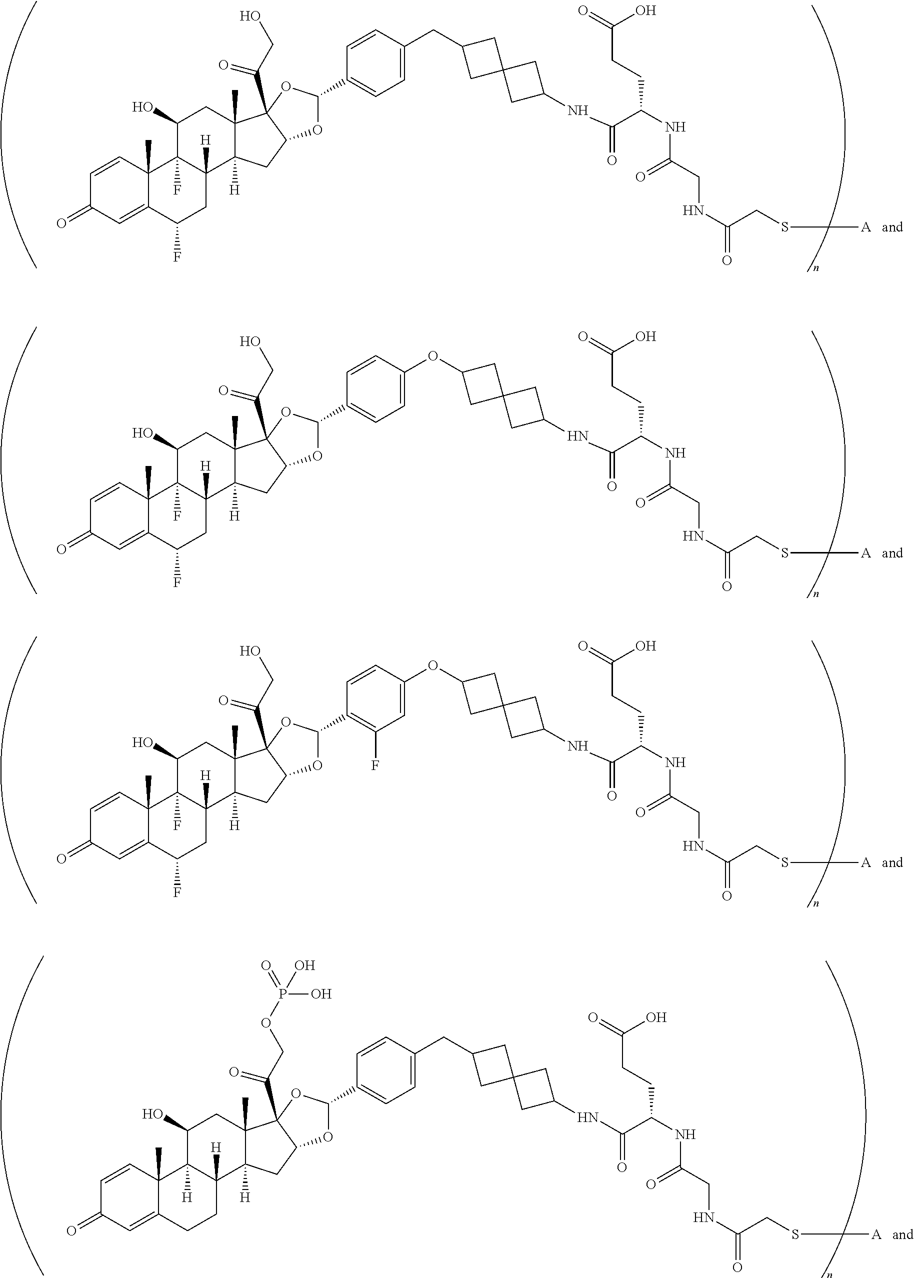

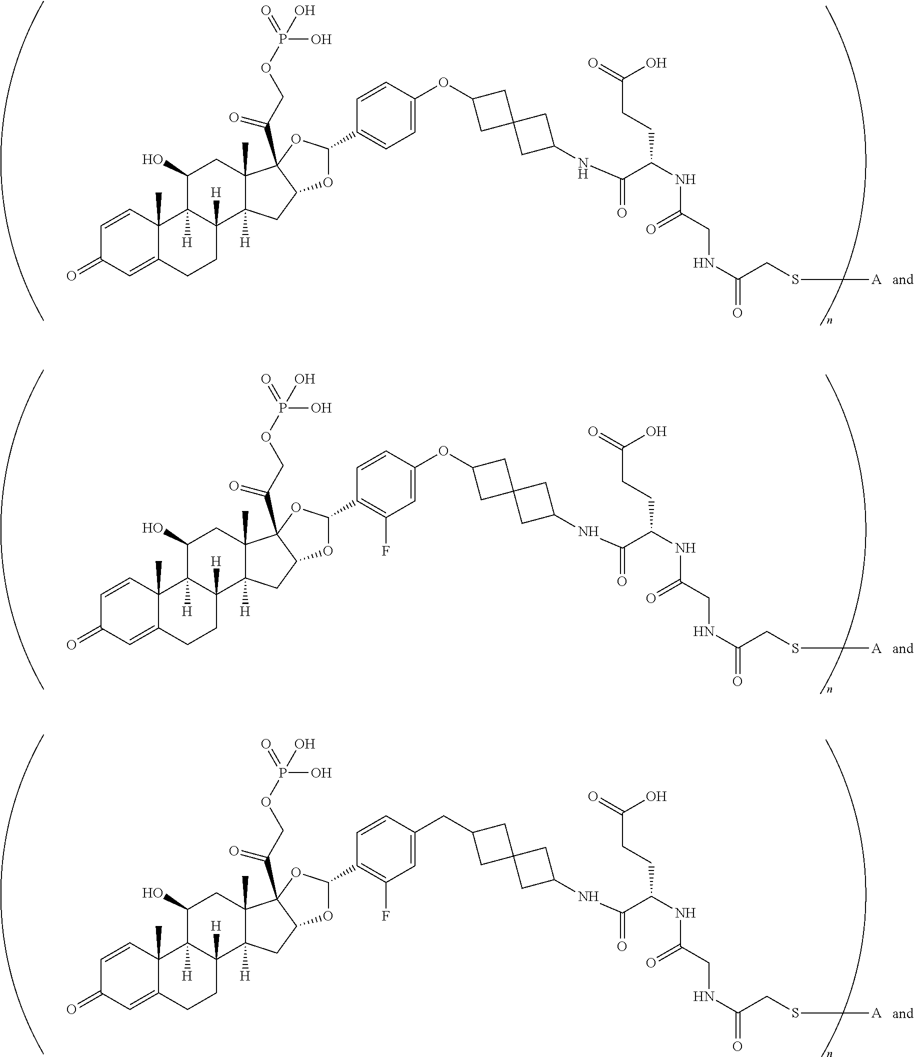

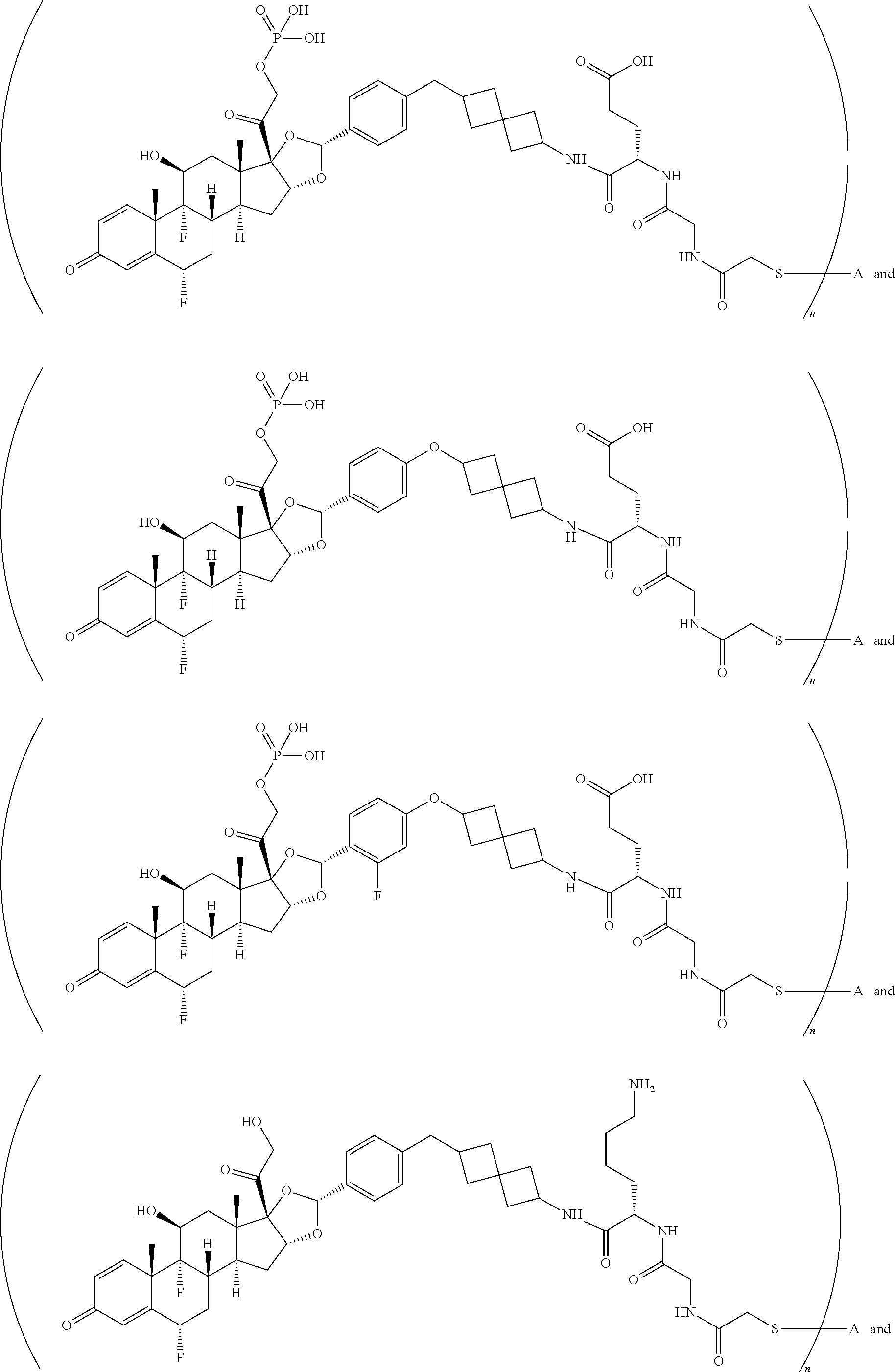

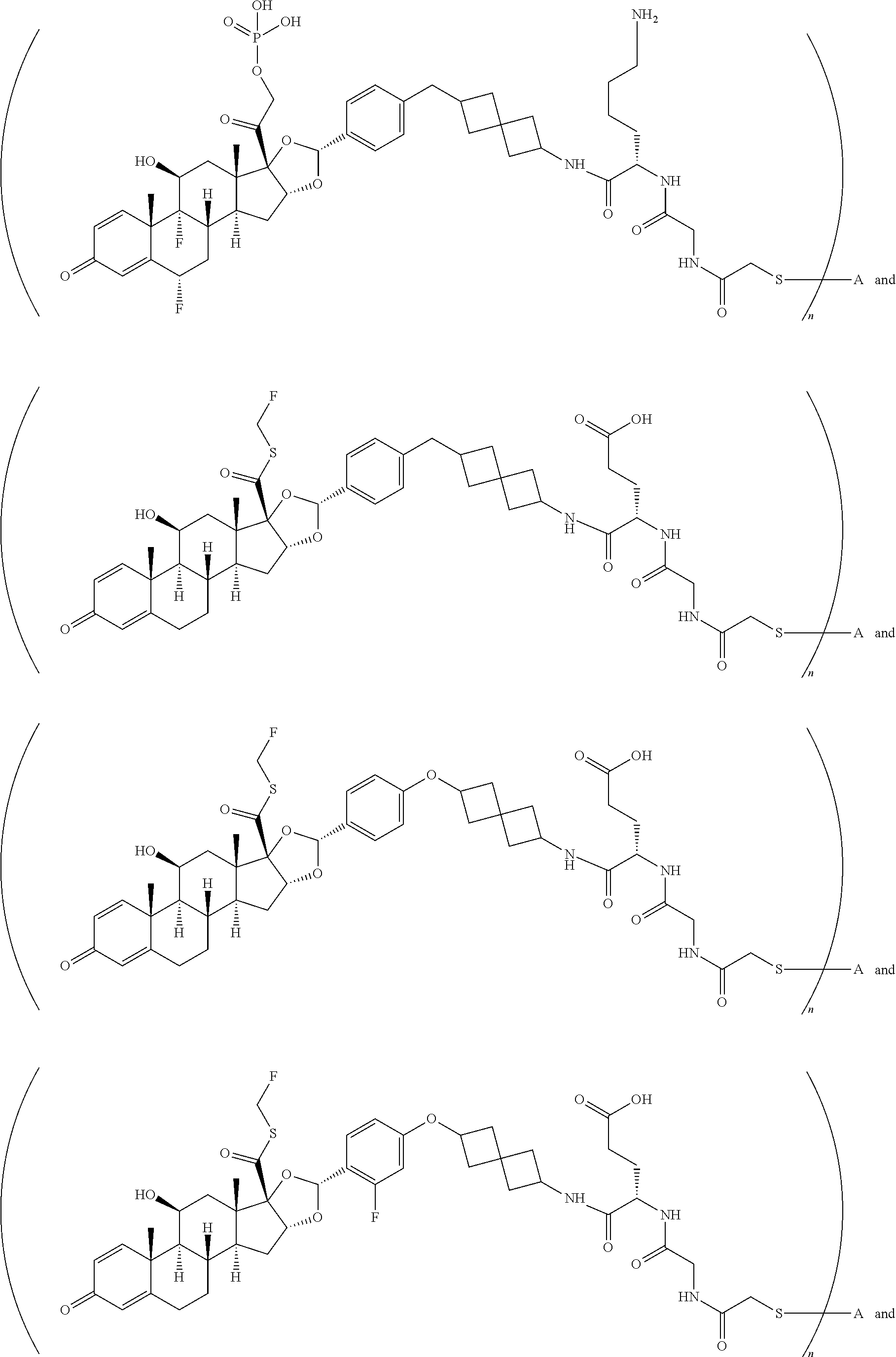

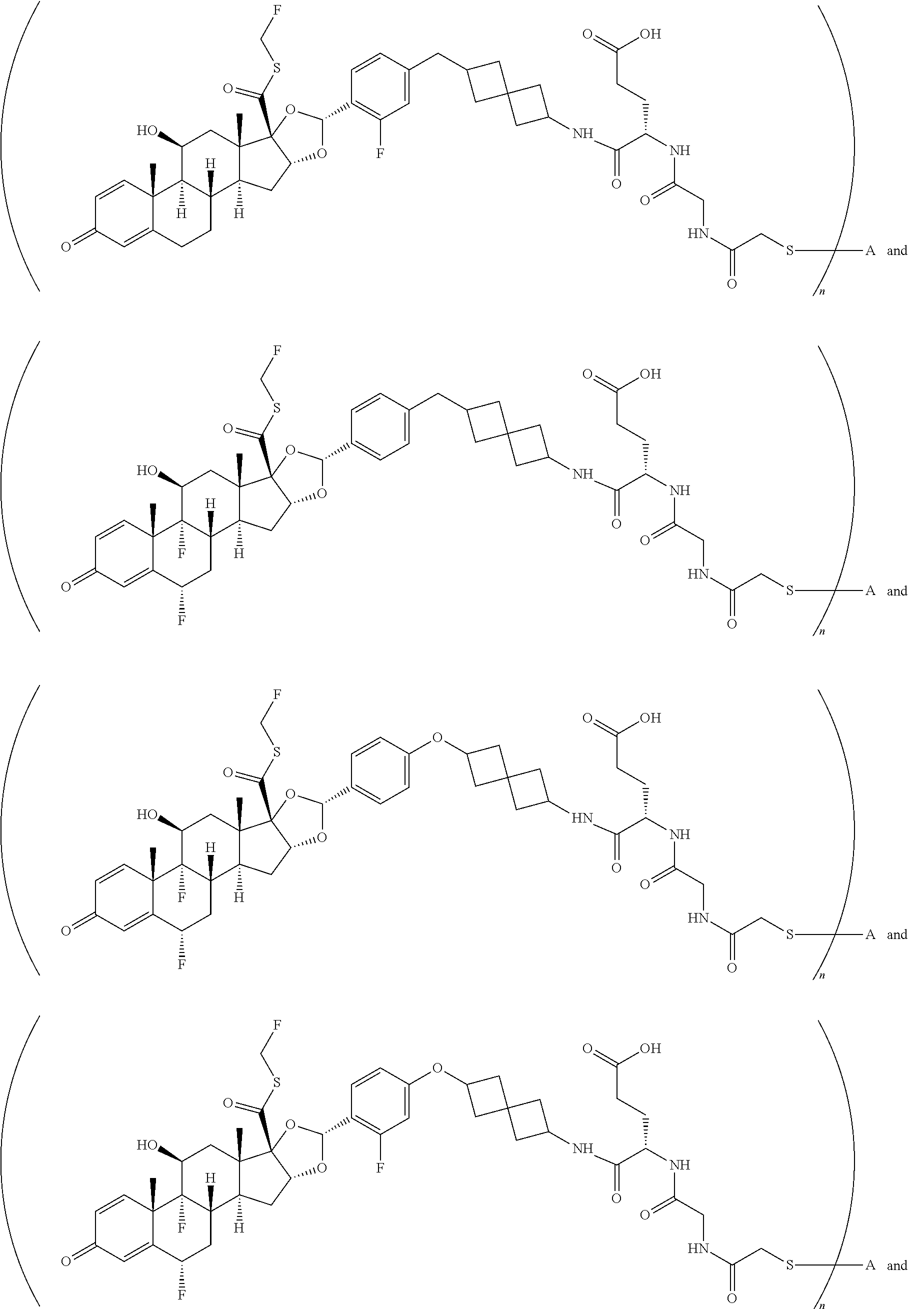

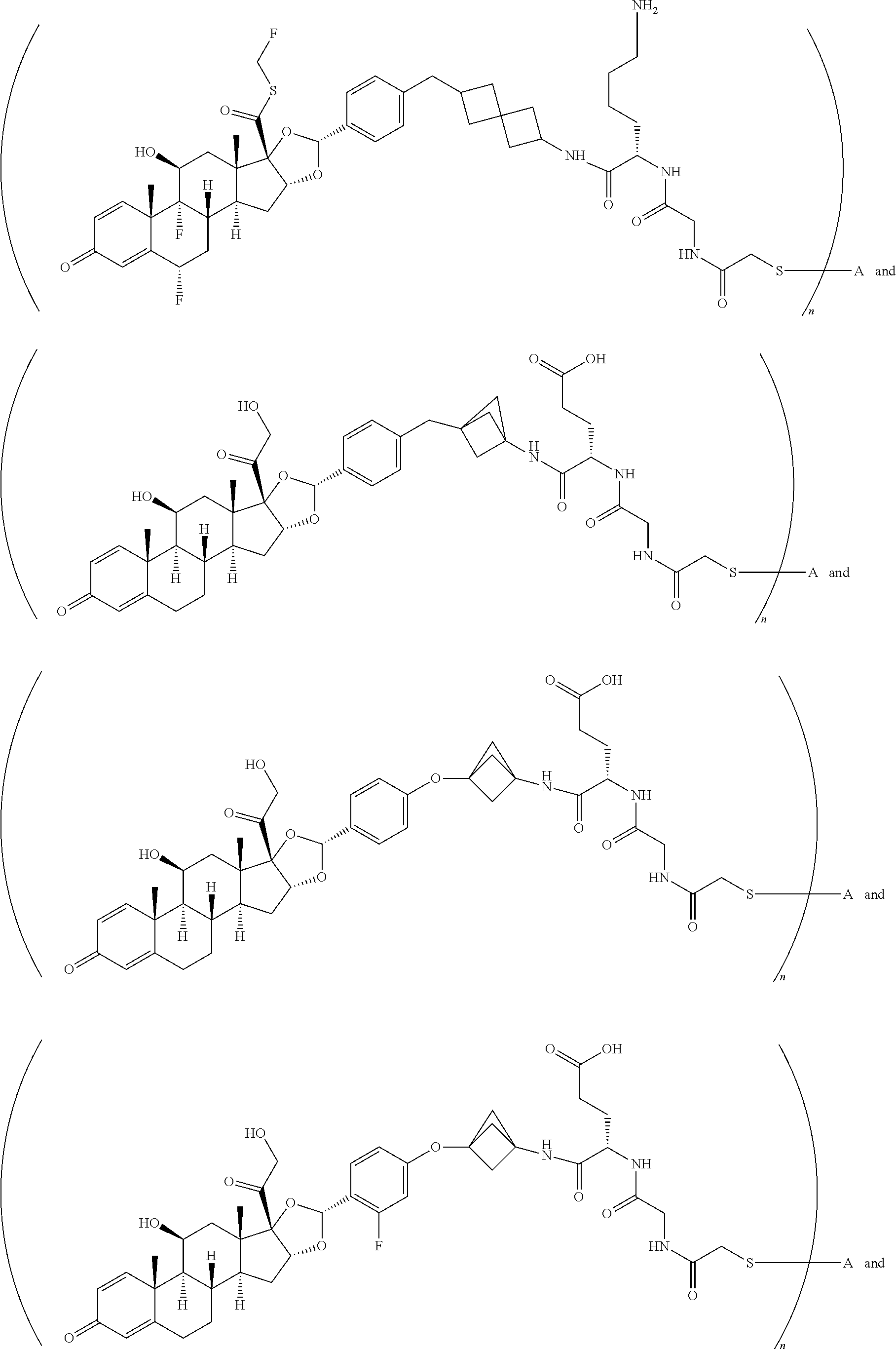

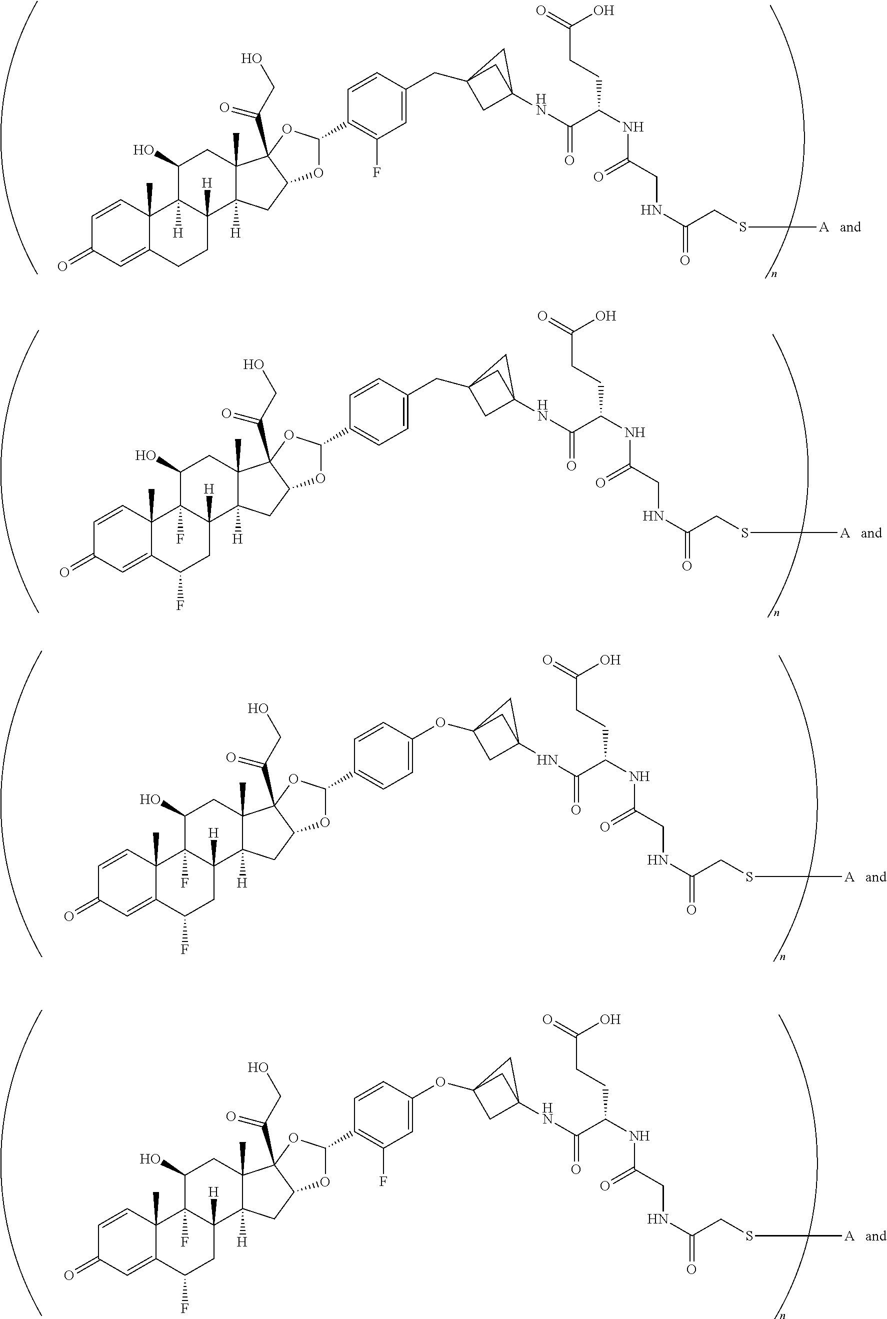

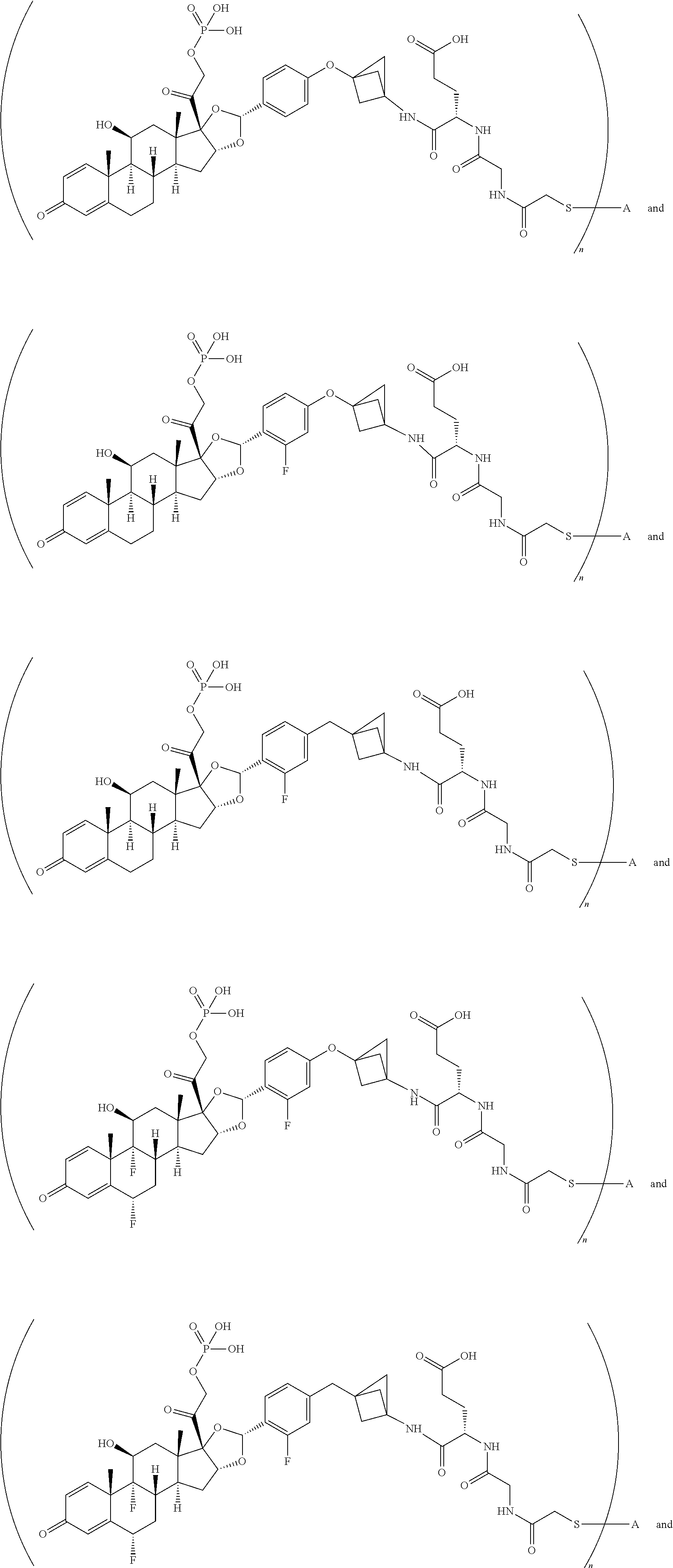

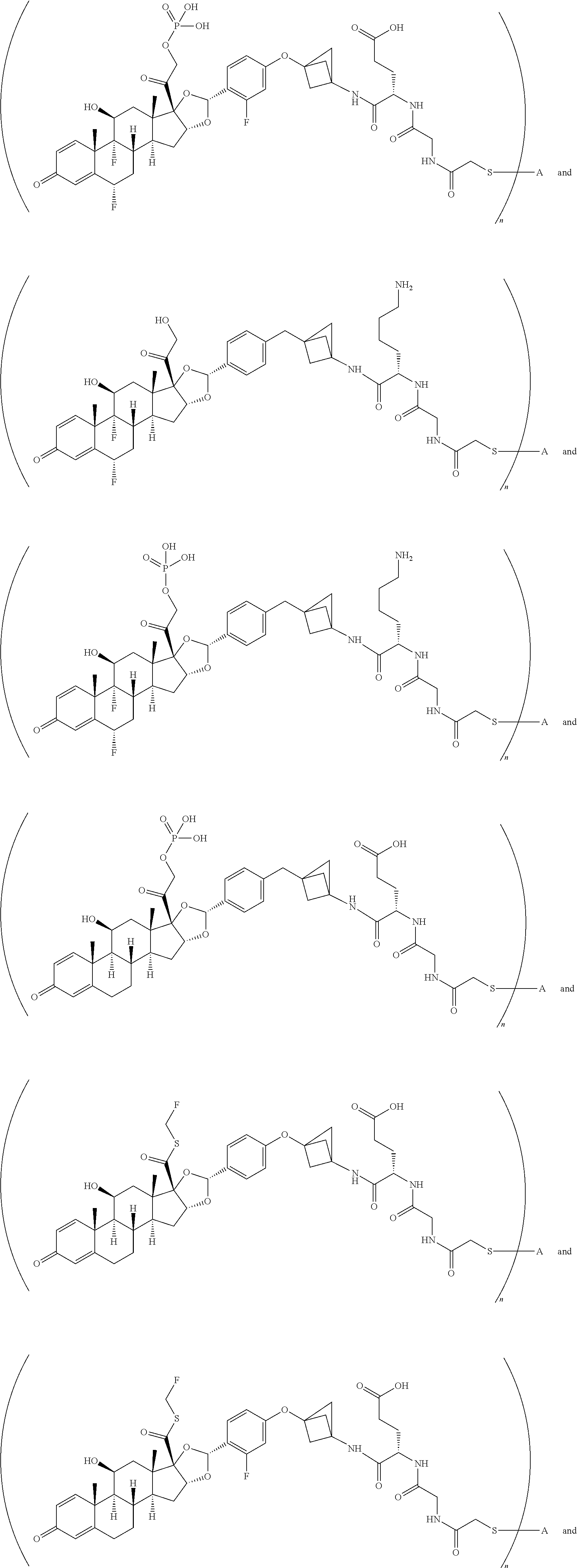

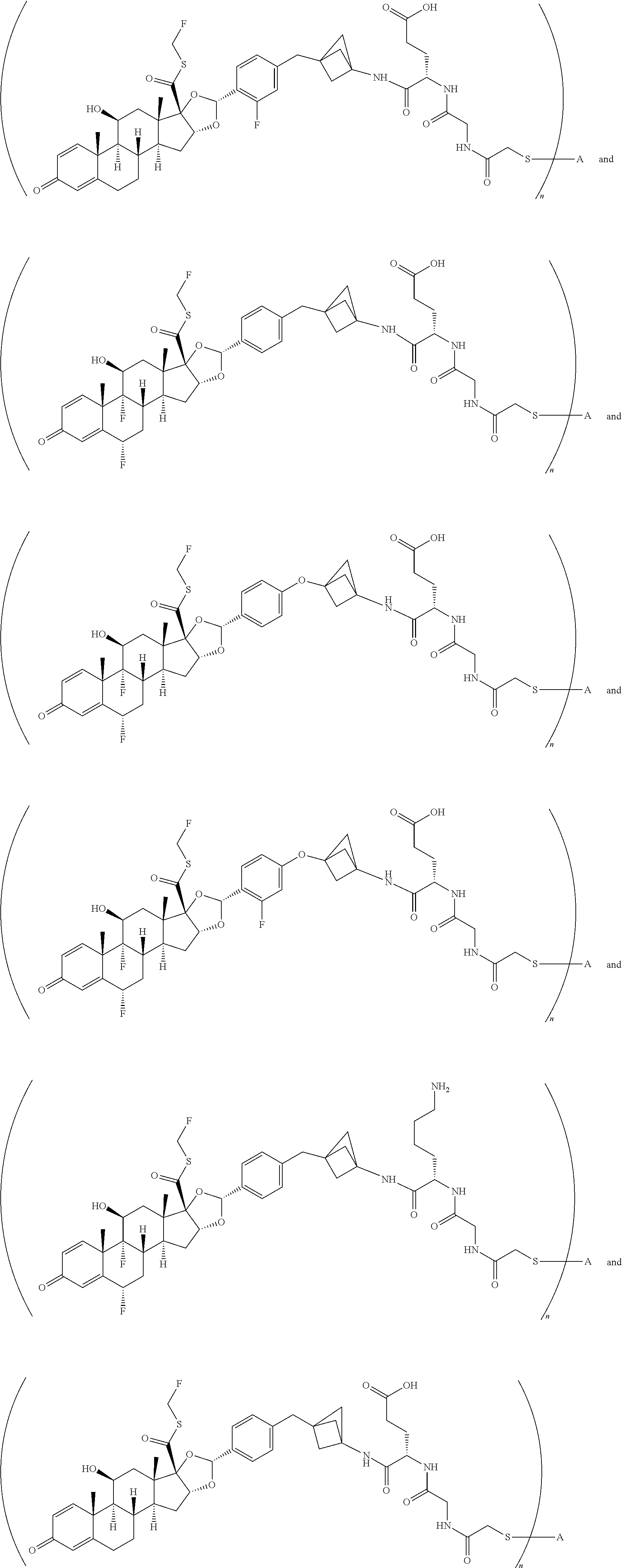

preferably where n=2-12, 2-10, 2-8, 2-6 or 2-4 and A is an antibody which binds to an antigen expressed by an immune cell, preferably a human immune cell and in some exemplary embodiments an anti-human VISTA antibody.

It is a specific object of the invention to provide a composition comprising at least one glucocorticoid agonist compound or steroid-linker conjugate of Formula I, II or III, or ADC containing according to any of the foregoing and a pharmaceutically acceptable carrier.

It is a specific object of the invention to provide a composition as set forth above which is suitable for in vivo administration to a subject in need thereof.

It is a specific object of the invention to provide a composition as set forth above, which comprises at least one excipient.

It is a specific object of the invention to provide a composition as set forth above, which comprises at least one stabilizer or buffer.

It is a specific object of the invention to provide a composition as set forth above which is suitable for parenteral administration, optionally by injection.

It is a specific object of the invention to provide a composition as set forth above which is suitable for injection to a subject in need thereof, optionally via intravenous, subcutaneous, intramuscular, intratumoral, or intrathecal administration.

It is a specific object of the invention to provide a composition as set forth above, which is subcutaneously, intramuscularly or intravenously administrable.

It is a specific object of the invention to provide a composition as set forth above, which is comprised in a device that provides for subcutaneous administration selected from the group consisting of a syringe, an injection device, an infusion pump, an injector pen, a needleless device, an autoinjector, and a subcutaneous patch delivery system, optionally a device which delivers to a patient a fixed dose of the glucocorticoid receptor agonist or ADC containing.

It is a specific object of the invention to provide the use of a glucocorticoid agonist compound or steroid-linker conjugate or ADC according to any of the foregoing, or a composition containing for treating, preventing or inhibiting inflammation, allergy or autoimmunity in a subject in need thereof.

It is a specific object of the invention to provide a glucocorticoid agonist compound or steroid-linker conjugate of Formula I, II or III, or ADC containing according to any of the foregoing, or a composition containing for use in the preparation of a medicament for treating, preventing or inhibiting inflammation or autoimmunity or an allergic reaction in a subject in need thereof.

It is a specific object of the invention to provide a method of treatment and/or prophylaxis, comprising administering to a patient in need thereof at least one glucocorticoid agonist compound or steroid-linker conjugate of Formula I, II or III, or ADC containing according to any of the foregoing, or a composition containing according to any of the foregoing.

It is a specific object of the invention to provide the use, medicament, composition or method of any of the foregoing, which is for the treatment of allergy, autoimmunity, transplant, gene therapy, inflammation, GVHD or sepsis, or to treat or prevent inflammatory, autoimmune, or allergic side effects associated with any of the foregoing conditions in a human subject.

It is a specific object of the invention to provide the use, medicament, composition or method of any of the foregoing, which is for acute use.

It is a specific object of the invention to provide the use, medicament, composition or method of any of the foregoing, which is for chronic use.

It is a specific object of the invention to provide the use, medicament, composition or method of any of the foregoing, which is for maintenance therapy.

It is a specific object of the invention to provide the use, medicament, composition or method of any of the foregoing, which is for the treatment or prophylaxis of acute or chronic inflammation and autoimmune and inflammatory indications associated therewith wherein the conditions optionally include Acquired aplastic anemia+, Acquired hemophilia+, Acute disseminated encephalomyelitis (ADEM)+, Acute hemorrhagic leukoencephalitis (AHLE)/Hurst's disease+, Agammaglobulinemia, primary+, Alopecia areata+, Ankylosing spondylitis (AS), Anti-NMDA receptor encephalitis+, Antiphospholipid syndrome (APS)+, Arteriosclerosis, Autism spectrum disorders (ASD), Autoimmune Addison's disease (AAD)+, Autoimmune dysautonomia/Autoimmune autonomic ganglionopathy (AAG), Autoimmune encephalitis+, Autoimmune gastritis, Autoimmune hemolytic anemia (AIHA)+, Autoimmune hepatitis (AIH)+, Autoimmune hyperlipidemia, Autoimmune hypophysitis/lymphocytic hypophysitis+, Autoimmune inner ear disease (AIED)+, Autoimmune lymphoproliferative syndrome (ALPS)+, Autoimmune myocarditis, Autoimmune oophoritis+, Autoimmune orchitis+, Autoimmune pancreatitis (AIP)/Immunoglobulin G4-Related Disease (IgG4-RD)+, Autoimmune polyglandular syndromes, Types I, II, & III+, Autoimmune progesterone dermatitis+, Autoimmune sudden sensorineural hearing loss (SNHL) Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Diabetes, type 1, Dermatitis herpetiformis, Dermatomyositis, Devic's disease (neuromyelitis optica). Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Fibrosing alveolitis, Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus (including nephritis and cutaneous), Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myelin Oligodendrocyte Glycoprotein Antibody Disorder, Myositis, Narcolepsy, Neonatal Lupus, Neuromyelitis optica, Neutropenia, Ocular cicatricial pemphigoid, Optic neuritis, Opsoclonus-myoclonus syndrome (OMS), Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary Biliary Cholangitis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Thyroid eye disease (TED), Tolosa-Hunt syndrome (THS), Transverse myelitis, Type 1 diabetes, Undifferentiated connective tissue disease (UCTD), Uveitis, Vasculitis, Vitiligo, Vogt-Koyanagi-Harada Disease, among others.

It is a specific object of the invention to provide the use, medicament, composition or method of any of the foregoing, which is for the treatment or prophylaxis of acute or chronic inflammation and autoimmune and inflammatory and allergic indications or side-effects associated therewith wherein the conditions optionally include Severe asthma, Giant cell arteritis, ANKA vasculitis and IBD (Colitis and Crohns).

It is a specific object of the invention to provide the use, medicament, composition or method of any of the foregoing, which is for the treatment or prophylaxis of a condition selected from rheumatoid arthritis, juvenile idiopathic arthritis, psoriatic arthritis, ankylosing spondylitis, adult Crohn's disease, pediatric Crohn's disease, ulcerative colitis, plaque psoriasis, hidradenitis suppurativa, uveitis, Bechet's disease, a spondyloarthropathy, or psoriasis.

It is a specific object of the invention to provide the use, medicament, composition or method of any of the foregoing, which is for treatment or prophylaxis in a patient who comprises one or more of the following:
(i) a chronic, acute, episodic allergic, inflammatory or inflammatory condition, e.g., a chronic, acute, episodic, and/or remitting/relapsing condition;
(ii) a condition primarily only effectively treatable with high doses of steroids, optionally asthma, COPD, polymyalgia rheumatica and/or giant cell arteritis, which patient optionally has been treated or is undergoing treatment with high steroid doses;
(iii) a condition with a comorbidity limiting steroid use, optionally diabetes mellitis, nonalcoholic steatohepatitis (NASH), morbid obesity, avascular necrosis/osteonecrosis (AVN), glaucoma, steroid-induced hypertension, severe skin fragility, and/or osteoarthritis;
(iv) a condition wherein safe long-term treatment agents are available, but wherein several months of induction with high-doses of steroids is desired, optionally AAV, polymyositis, dermamyositis, lupus, inflammatory lung disease, autoimmune hepatitis, inflammatory bowel disease, immune thrombocytopenia, autoimmune hemolytic anemia, gout patients wherein several months of induction with high-doses of steroids is therapeutically warranted;
(v) dermatologic conditions that require short/long-term treatment, optionally of uncertain treatment or duration and/or no effective alternative to steroid administration, optionally Stevens Johnson, other severe drug eruption conditions, conditions involving extensive contact dermatitis, other severe immune-related dermatological conditions such as PG, LCV, Erythroderma and the like;
(vi) conditions treated with high-dose corticosteroids for flares/reoccurrences, optionally COPD, asthma, lupus, gout, pseudogout;
(vii) immune-related neurologic diseases such as small-fiber neuropathy, MS (subset), chronic inflammatory demyelinating polyneuropathy, myasthenia gravis and the like;
(viii) hematological/oncology indications, optionally wherein high doses of steroids would potentially be therapeutically warranted or beneficial;
(ix) ophthalmologic conditions, optionally uveitis, iritis, scleritis, and the like;
(x) conditions associated with permanent or very prolonged adrenal insufficiency or secondary adrenal insufficiency, optionally latrogenic Addisonian crisis;
(xi) conditions often treated with long term, low dose steroids, optionally lupus, RA, psA, vasculitis, and the like; or
(xii) any combination of any of the foregoing.

It is a specific object of the invention to provide the use, medicament, composition or method of any of the foregoing, which is for treatment or prophylaxis in a patient who is in a special class of patients who are at risk of toxicity in steroid treatment such as pregnant/breast-feeding women, pediatric patients optionally those with growth impairment or cataracts, wherein the patient is further being treated with another active agent.

It is a specific object of the invention to provide the use, medicament, composition or method of any of the foregoing, wherein the patient is further being treated with an immunomodulatory antibody or fusion protein which optionally is selected from immmunoinhibitory antibodies or fusion proteins targeting one or more of CTLA4, PD-1, PDL-1, LAG-3, TIM-3, BTLA, B7-H4, B7-H3, VISTA, and/or agonistic antibodies or fusion protein targeting one or more of CD40, CD137, OX40, GITR, CD27, CD28 or ICOS.

It is a specific object of the invention to provide the use, medicament, composition or method of any of the foregoing, wherein the ADC comprises an antibody or antigen binding fragment comprising an antigen binding region that specifically binds to human V-domain Ig Suppressor of T cell Activation (human VISTA) (“A”), wherein the ADC, when administered to a subject in need thereof, is preferentially delivered to VISTA expressing immune cells, optionally one or more of monocytes, myeloid cells, T cells, Tregs, NK cells, Neutrophils, dendritic cells, eosinophils, macrophages, NK cells, and endothelial cells, and results in the functional internalization of the anti-inflammatory agent into one or more of said immune cells.

It is a specific object of the invention to provide the use, medicament, composition or method of any of the foregoing, wherein the ADC comprises an anti-human VISTA antibody or antibody fragment that preferentially binds to VISTA expressing cells at physiological pH (≈7.5); which optionally has a pK of at most 70 hours in a human VISTA knock-in rodent.

It is a specific object of the invention to provide the use, medicament, composition or method of any of the foregoing, wherein the ADC comprises an anti-human VISTA antibody or antibody fragment, which has a pK of at most 3.5±5 days, more typically at most 48 hours, at most 36 hours, at most 24 hours or at most 18 hours or at most 12 hours in a Cynomolgus macaque or a human at physiologic pH.

It is a specific object of the invention to provide the use, medicament, composition or method of any of the foregoing, wherein the ADC comprises an anti-human VISTA antibody or antibody fragment, which has a pK of at most 2.8 or 2.3 or 1.5 days or 1 day or 12 hours or 8 hours±0.5 days in Cynomolgus macaque or in a human at physiologic pH.

It is a specific object of the invention to provide the use, medicament, composition or method of any of the foregoing, wherein the ADC comprises an anti-human VISTA antibody or antibody fragment, which has a pK of at most 6-12 hours in a human VISTA rodent at physiologic pH.

It is a specific object of the invention to provide the use, medicament, composition or method of any of the foregoing, wherein the ADC comprises an anti-human VISTA antibody or antibody fragment, which comprises a linker which upon internalization of the ADC into VISTA-expressing immune cells, optionally one or more of activated or non-activated T cells, CD4 or CD8 T cells, Tregs, NK cells, Neutrophils, monocytes, myeloid cells, dendritic cells, NK cells, macrophages, eosinophils, and endothelial cells, is cleaved resulting in the release of a therapeutically effective amount of the anti-inflammatory agent (glucocorticoid agonist) in the immune cell, wherein it elicits anti-inflammatory activity.

It is a specific object of the invention to provide the use, medicament, composition or method of any of the foregoing, wherein the ADC comprises an anti-human VISTA antibody or antibody fragment the anti-VISTA antibody or antigen binding fragment has an in vivo serum half-life of about 2.3 days in a primate, optionally Cynomolgus macaque at physiological pH (˜pH 7.5).

It is a specific object of the invention to provide the use, medicament, composition or method of any of the foregoing, wherein the ADC comprises an anti-human VISTA antibody or antibody fragment, wherein the anti-VISTA antibody or antigen binding fragment has an in vivo serum half-life in serum at physiological pH (˜pH 7.5) in a human VISTA knock-in rodent of no more than 70 hours, no more than 60 hours, no more than 50 hours, no more than 40 hours, no more than 30 hours, no more than 24 hours, no more than 22-24 hours, no more than 20-22 hours, no more than 18-20 hours, no more than 16-18 hours, no more than 14-16 hours, no more than 12-14 hours, no more than 10-12 hours, no more than 8-10 hours, no more than 6-8 hours, no more than 4-6 hours, no more than 2-4 hours, no more than 1-2 hours, no more than 0.5 to 1.0 hours, or no more than 0.1-0.5 hours.

It is a specific object of the invention to provide the use, medicament, composition or method of any of the foregoing, wherein the ADC comprises an anti-human VISTA antibody or antibody fragment, wherein the PD/PK ratio of the ADC when used in vivo is at least 2:1, 3:1, 4:1, 5:1, 6:1, 7:1, 8:1, 9:1, 10:1, 11:1, 12:1, 13:1, 14:1 or greater in a human VISTA knock-in rodent or in a human or non-human primate, optionally Cynomolgus macaque.

It is a specific object of the invention to provide the use, medicament, composition or method of any of the foregoing, wherein the ADC comprises an anti-human VISTA antibody or antibody fragment, wherein the PD of the ADC is at least 4, 5, 6, 7, 8, 9, 10, 11, 12, 13, or 14 days, 2-4 weeks, a month or longer in any one of a rodent or in a human or non-human primate, optionally Cynomolgus macaque.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, wherein the antibody comprises an Fc region having impaired FcR binding or intact FcR binding.

It is a specific object of the invention to provide the use, medicament, composition or method of any of the foregoing, wherein the ADC comprises an antibody or antibody fragment targets a human immune cell expressed antigen, e.g., an anti-human VISTA antibody or antibody fragment, which comprises a human IgG1, IgG2, IgG3 or IgG4 Fc region having impaired FcR binding or intact FcR binding.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, which comprises a human IgG1 Fc region having impaired FcR binding.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, which comprises a human or non-human primate constant or Fc region which is modified to impair or eliminate binding to at least 2 native human Fc gamma receptors.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, which comprises a human or non-human primate constant or Fc region modified to impair or eliminate binding to any one, two, three, four or all five of the following FcRs: hFcγRI (CD64), FcyRIIA or hFcyRIIB, (CD32 or CD32A) and FcγRIIIA (CD16A) or FcγRIIIB (CD16B).

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, which comprises a human IgG2 kappa backbone, optionally with V234A/G237A/P238S/H268A/V309L/A330S/P331S silencing mutations in the Fc region.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, which comprises a human IgG1/kappa backbone with L234A/L235A silencing mutations in the Fc region and optionally a mutation which impairs complement (C1Q) binding.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, which comprises a human IgG1/kappa backbone, optionally with L234A/L235A silencing mutations and E269R and E233A mutations in the Fc region.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, wherein the binding of the antibody or antigen binding fragment to immune cells does not directly agonize or antagonize said immune cell expressed antigen mediated effects on immunity, e.g., VISTA-mediated effects on immunity.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, comprising a human IgG1, IgG2, IgG3 or IgG4 Fc region wherein endogenous FcR binding is not impaired.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, comprising a native (unmodified) human IgG2 Fc region.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, wherein the antibody or antigen binding fragment comprises a KD ranging from 0.0001 nM to 10.0 nM, 0.001 to 1.0 nM, or 0.01 to 0.7 or less determined by surface plasmon resonance (SPR) at 24° C. or 37° C.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, wherein the antibody or antigen binding fragment comprises a KD of 0.13 to 0.64 nM determined by surface plasmon resonance (SPR) at 24° C. or 37° C.

It is a specific object of the invention to provide the use, medicament, composition or method of any of the foregoing, wherein the ADC optionally comprises an antibody or antibody fragment that specifically binds to a human immune cell expressed antigen, e.g., an anti-human VISTA antibody or antibody fragment, wherein the drug antibody ratio ranges from about 1:1-12:1.

It is a specific object of the invention to provide the use, medicament, composition or method of any of the foregoing, wherein the ADC optionally comprises an anti-human VISTA antibody or antibody fragment, wherein the drug antibody ratio ranges from about 2-12:1, 2-8:1, 4-8:1, or 6-8:1.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, wherein the drug antibody ratio the drug antibody ratio is about 8:1 (n=8) or is about 4:1 (n=4).

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, which internalizes one or more of monocytes, myeloid cells, T cells, Tregs, macrophages and neutrophils.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment which does not appreciably internalize B cells.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, when administered to a subject in need thereof promotes the efficacy and/or reduces adverse side effects such as toxicity associated with the anti-inflammatory agent, compared to the same dosage of anti-inflammatory agent administered in naked (non-conjugated) form.

It is a specific object of the invention to provide the use, medicament, composition or method of any of the foregoing, wherein the ADC optionally comprises an antibody or antibody fragment that specifically binds to a human immune cell expressed antigen, e.g., an anti-human VISTA antibody or antibody fragment, wherein the glucocorticoid is optionally conjugated to the antibody or antigen-binding fragment via the interchain disulfides.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, which comprises an esterase sensitive linker.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment, wherein the cleavable linker is susceptible to one or more of acid-induced cleavage, photo-induced cleavage, peptidase-induced cleavage, esterase-induced cleavage, and disulfide bond cleavage.

It is a specific object of the invention to provide the use, medicament, composition or method of any of the foregoing, wherein the ADC comprises an antibody or antibody fragment that specifically binds to a human immune cell expressed antigen, e.g., an anti-human VISTA antibody or antibody fragment wherein the anti-VISTA antigen binding fragment comprised in the ADC comprises a Fab, F(ab′)2, or scFv antibody fragment.

It is a specific object of the invention to provide the use, medicament, composition or method of any of the foregoing, wherein the ADC comprises an anti-human VISTA antibody or antibody fragment, wherein the anti-VISTA antibody or antibody fragment contained therein is one which comprises the same CDRs as an antibody having the sequences in FIG. 8, 10 or 12 or is optionally selected from one that:
(i) comprises the V_{H} CDRs of SEQ ID NO: 100, 101 and 102 and the V_{L} CDRs of SEQ ID NO:103, 104 and 105;
(ii) comprises the V_{H} CDRs of SEQ ID NO: 110, 111 and 112 and the V_{L} CDRs of SEQ ID NO: 113, 114 and 115;
(iii) comprises the V_{H} CDRs of SEQ ID NO: 120, 121 and 122 and the V_{L} CDRs of SEQ ID NO: 123, 124 and 125;
(iv) comprises the V_{H} CDRs of SEQ ID NO: 130, 131 and 132 and the V_{L} CDRs of SEQ ID NO: 133, 134 and 135;
(v) comprises the V_{H} CDRs of SEQ ID NO: 140, 141 and 142 and the V_{L} CDRs of SEQ ID NO: 143, 144 and 145;
(vi) comprises the V_{H} CDRs of SEQ ID NO: 150, 151 and 152 and the V_{L} CDRs of SEQ ID NO: 153, 154 and 155;
(vii) comprises the V_{H} CDRs of SEQ ID NO: 160, 161 and 162 and the V_{L} CDRs of SEQ ID NO: 163, 164 and 165;
(viii) comprises the V_{H} CDRs of SEQ ID NO: 170, 171 and 172 and the V_{L} CDRs of SEQ ID NO:173, 174 and 175;
(ix) comprises the V_{H} CDRs of SEQ ID NO: 180, 181 and 182 and the V_{L} CDRs of SEQ ID NO: 183, 184 and 185;
(x) comprises the V_{H} CDRs of SEQ ID NO: 190, 191 and 192 and the V_{L} CDRs of SEQ ID NO: 193, 194 and 195;
(xi) comprises the V_{H} CDRs of SEQ ID NO:200, 201 and 202 and the V_{L} CDRs of SEQ ID NO:203, 204 and 205;
(xii) comprises the V_{H} CDRs of SEQ ID NO:210, 211 and 212 and the V_{L} CDRs of SEQ ID NO:213, 214 and 215;
(xiii) comprises the V_{H} CDRs of SEQ ID NO:220, 221 and 222 and the V_{L} CDRs of SEQ ID NO:223, 224 and 225;
(xiv) comprises the V_{H} CDRs of SEQ ID NO:230, 231 and 232 and the V_{L} CDRs of SEQ ID NO:233, 234 and 235;
(xv) comprises the V_{H} CDRs of SEQ ID NO:240, 241 and 242 and the V_{L} CDRs of SEQ ID NO:243, 244 and 245;
(xvi) comprises the V_{H} CDRs of SEQ ID NO:250, 251 and 252 and the V_{L} CDRs of SEQ ID NO:253, 254 and 255;
(xvii) comprises the V_{H} CDRs of SEQ ID NO:260, 261 and 262 and the V_{L} CDRs of SEQ ID NO:263, 264 and 265;
(xviii) comprises the V_{H} CDRs of SEQ ID NO:270, 271 and 272 and the V_{L} CDRs of SEQ ID NO:273, 274 and 275;
(xix) comprises the V_{H} CDRs of SEQ ID NO:280, 281 and 282 and the V_{L} CDRs of SEQ ID NO:283, 284 and 285;
(xx) comprises the V_{H} CDRs of SEQ ID NO:290, 291 and 292 and the V_{L} CDRs of SEQ ID NO:293, 294 and 295;
(xxi) comprises the V_{H} CDRs of SEQ ID NO:300, 301 and 302 and the V_{L} CDRs of SEQ ID NO:303, 304 and 305;
(xxii) comprises the V_{H} CDRs of SEQ ID NO:310, 311 and 312 and the V_{L} CDRs of SEQ ID NO:313, 314 and 315;
(xxiii) comprises the V_{H} CDRs of SEQ ID NO:320, 321 and 322 and the V_{L} CDRs of SEQ ID NO:323, 324 and 325;

(xxiv) comprises the V_{H} CDRs of SEQ ID NO:330, 331 and 332 and the V_{L} CDRs of SEQ ID NO:333, 334 and 335;
(xxv) comprises the V_{H} CDRs of SEQ ID NO:340, 341 and 342 and the V_{L} CDRs of SEQ ID NO:343, 344 and 345;
(xxvi) comprises the V_{H} CDRs of SEQ ID NO:350, 351 and 352 and the V_{L} CDRs of SEQ ID NO:353, 354 and 355;
(xxvii) comprises the V_{H} CDRs of SEQ ID NO:360, 361 and 362 and the V_{L} CDRs of SEQ ID NO:363, 364 and 365;
(xxviii) comprises the V_{H} CDRs of SEQ ID NO:370, 371 and 372 and the V_{L} CDRs of SEQ ID NO:373, 374 and 375;
(xxix) comprises the V_{H} CDRs of SEQ ID NO:380, 381 and 382 and the V_{L} CDRs of SEQ ID NO:383, 384 and 385;
(xxx) comprises the V_{H} CDRs of SEQ ID NO:390, 391 and 392 and the V_{L} CDRs of SEQ ID NO:393, 394 and 395;
(xxxi) comprises the V_{H} CDRs of SEQ ID NO:400, 401 and 402 and the V_{L} CDRs of SEQ ID NO:403, 404 and 405;
(xxxii) comprises the V_{H} CDRs of SEQ ID NO:410, 411 and 412 and the V_{L} CDRs of SEQ ID NO:413, 414 and 415;
(xxxiii) comprises the V_{H} CDRs of SEQ ID NO:420, 421 and 422 and the V_{L} CDRs of SEQ ID NO:423, 424 and 425;
(xxxiv) comprises the V_{H} CDRs of SEQ ID NO:430, 431 and 432 and the V_{L} CDRs of SEQ ID NO:433, 434 and 435;
(xxxv) comprises the V_{H} CDRs of SEQ ID NO:440, 441 and 442 and the V_{L} CDRs of SEQ ID NO:443, 444 and 445;
(xxxvi) comprises the V_{H} CDRs of SEQ ID NO:450, 451 and 452 and the V_{L} CDRs of SEQ ID NO:453, 454 and 455;
(xxxvii) comprises the V_{H} CDRs of SEQ ID NO:460, 461 and 462 and the V_{L} CDRs of SEQ ID NO:463, 464 and 465;
(xxxviii) comprises the V_{H} CDRs of SEQ ID NO:470, 471 and 472 and the V_{L} CDRs of SEQ ID NO:473, 474 and 475;
(xxxix) comprises the V_{H} CDRs of SEQ ID NO:480, 481 and 482 and the V_{L} CDRs of SEQ ID NO:483, 484 and 485;
(xl) comprises the V_{H} CDRs of SEQ ID NO:490, 491 and 492 and the V_{L} CDR polypeptides of SEQ ID NO:493, 494 and 495;
(xli) comprises the V_{H} CDRs of SEQ ID NO:500, 501 and 502 and the V_{L} CDR polypeptides of SEQ ID NO:503, 504 and 505;
(xlii) comprises the V_{H} CDRs of SEQ ID NO:510, 511 and 512 and the V_{L} CDR polypeptides of SEQ ID NO:513, 514 and 515;
(xliii) comprises the V_{H} CDRs of SEQ ID NO:520, 521 and 522 and the V_{L} CDR polypeptides of SEQ ID NO:523, 524 and 525;
(xliv) comprises the V_{H} CDRs of SEQ ID NO:530, 531 and 532 and the V_{L} CDR polypeptides of SEQ ID NO:533, 534 and 535;
(xlv) comprises the V_{H} CDRs of SEQ ID NO:540, 541 and 542 and the V_{L} CDR polypeptides of SEQ ID NO:543, 544 and 545;
(xlvi) comprises the V_{H} CDRs of SEQ ID NO:550, 551 and 552 and the V_{L} CDR polypeptides of SEQ ID NO:553, 554 and 555;
(xlvii) comprises the V_{H} CDRs of SEQ ID NO:560, 561 and 562 and the V_{L} CDRs of SEQ ID NO:563, 564 and 565;
(xlviii) comprises the V_{H} CDRs of SEQ ID NO:570, 571 and 572 and the V_{L} CDRs of SEQ ID NO:573, 574 and 575;
(xlix) comprises the V_{H} CDRs of SEQ ID NO:580, 581 and 582 and the V_{L} CDRs of SEQ ID NO:583, 584 and 585;
(l) comprises the V_{H} CDRs of SEQ ID NO:590, 591 and 592 and the V_{L} CDRs of SEQ ID NO:593, 594 and 595;
(li) comprises the V_{H} CDRs of SEQ ID NO:600, 601 and 602 and the V_{L} CDRs of SEQ ID NO:603, 604 and 605;
(lii) comprises the V_{H} CDRs of SEQ ID NO:610, 611 and 612 and the V_{L} CDRs of SEQ ID NO:613, 614 and 615;
(liii) comprises the V_{H} CDRs of SEQ ID NO:620, 621 and 622 and the V_{L} CDRs of SEQ ID NO:623, 624 and 625;
(liv) comprises the V_{H} CDRs of SEQ ID NO:630, 631 and 632 and the V_{L} CDRs of SEQ ID NO:633, 634 and 635;
(lv) comprises the V_{H} CDRs of SEQ ID NO:640, 641 and 642 and the V_{L} CDRs of SEQ ID NO:643, 644 and 645;
(lvi) comprises the V_{H} CDRs of SEQ ID NO:650, 651 and 652 and the V_{L} CDRs of SEQ ID NO:653, 654 and 655;
(lvii) comprises the V_{H} CDRs of SEQ ID NO:660, 661 and 662 and the V_{L} CDRs of SEQ ID NO:663, 664 and 665;
(lviii) comprises the V_{H} CDRs of SEQ ID NO:670, 671 and 672 and the V_{L} CDRs of SEQ ID NO:673, 674 and 675;
(lix) comprises the V_{H} CDRs of SEQ ID NO:680, 681 and 682 and the V_{L} CDRs of SEQ ID NO:683, 684 and 685;
(lx) comprises the V_{H} CDRs of SEQ ID NO:690, 691 and 692 and the V_{L} CDRs of SEQ ID NO:693, 694 and 695;
(lxi) comprises the V_{H} CDRs of SEQ ID NO:700, 701 and 702 and the V_{L} CDRs of SEQ ID NO:703, 704 and 705;
(lxii) comprises the V_{H} CDRs of SEQ ID NO:710, 711 and 712 and the V_{L} CDRs of SEQ ID NO:713, 714 and 715;
(lxiii) comprises the V_{H} CDRs of SEQ ID NO:720, 721 and 722 and the V_{L} CDRs of SEQ ID NO:723, 724 and 725;
(lxiv) comprises the V_{H} CDRs of SEQ ID NO:730, 731 and 732 and the V_{L} CDRs of SEQ ID NO:733, 734 and 735;
(lxv) comprises the V_{H} CDRs of SEQ ID NO:740, 741 and 742 and the V_{L} CDRs of SEQ ID NO:743, 744 and 745;
(lxvi) comprises the V_{H} CDRs of SEQ ID NO:750, 751 and 752 and the V_{L} CDRs of SEQ ID NO:753, 754 and 755;
(lxvii) comprises the V_{H} CDRs of SEQ ID NO: 760, 761 and 762 and the V_{L} CDRs of SEQ ID NO:763, 764 and 765;
(lxviii) comprises the V_{H} CDRs of SEQ ID NO:770, 771 and 772 and the V_{L} CDRs of SEQ ID NO:773, 774 and 775; (lxix) comprises the V_{H} CDRs of SEQ ID NO: 780, 781 and 782 and the V_{L} CDRs of SEQ ID NO:783, 784 and 785;
(lxx) comprises the V_{H} CDRs of SEQ ID NO:790, 791 and 792 and the V_{L} CDRs of SEQ ID NO:793, 794 and 795;
(lxxi) comprises the V_{H} CDRs of SEQ ID NO:800, 801 and 802 and the V_{L} CDRs of SEQ ID NO:803, 804 and 805;
(lxxii) comprises the V_{H} CDRs of SEQ ID NO:810, 811 and 812 and the V_{L} CDRs of SEQ ID NO: 813, 814 and 815.

It is a specific object of the invention to provide an antibody drug conjugate (ADC) of any one of the foregoing, wherein the ADC comprises an anti-VISTA antibody or antibody fragment that comprises the same CDRS as any one of VSTB92, VSTB56, VSTB95, VSTB103 and VSTB66.

It is a specific object of the invention to an antibody drug conjugate (ADC) of any one of the foregoing wherein the ADC comprises an anti-VISTA antibody or antibody fragment that comprises a V_{H} polypeptide and a V_{L} polypeptide which respectively possess at least 90%, 95% or 100% sequence identity to those of an antibody comprising the following V_{H} polypeptide and a V_{L} polypeptides and further wherein the CDRs are not modified:
(i) one comprising the V_{H} polypeptide of SEQ ID NO: 106 identity and the V_{L} polypeptide of SEQ ID NO: 108;
(ii) one comprising the V_{H} polypeptide of SEQ ID NO: 116 and the V_{L} polypeptide of SEQ ID NO:118;
(iii) one comprising the V_{H} polypeptide of SEQ ID NO: 126 and the V_{L} polypeptide of SEQ ID NO:128;
(iv) one comprising the V_{H} polypeptide of SEQ ID NO: 136 and the V_{L} polypeptide f SEQ ID NO: 138;
(v) one comprising the V_{H} polypeptide of SEQ ID NO: 146 and the V_{L} polypeptide of SEQ ID NO:148;
(vi) one comprising the V_{H} polypeptide of SEQ ID NO: 156 and the V_{L} polypeptide of SEQ ID NO:158;
(vii) one comprising the V_{H} polypeptide of SEQ ID NO: 166 and the V_{L} polypeptide of SEQ ID NO:168;
(viii) one comprising the V_{H} polypeptide of SEQ ID NO: 176 and the V_{L} polypeptide of SEQ ID NO:178;
(ix) one comprising the V_{H} polypeptide of SEQ ID NO: 186 and the V_{L} polypeptide of SEQ ID NO:188;
(x) one comprising the V_{H} polypeptide of SEQ ID NO: 196 and the V_{L} polypeptide of SEQ ID NO:198;
(xi) one comprising the V_{H} polypeptide of SEQ ID NO:206 and the V_{L} polypeptide of SEQ ID NO:208;
(xii) one comprising the V_{H} polypeptide of SEQ ID NO:216 and the V_{L} polypeptide of SEQ ID NO:218;
(xiii) one comprising the V_{H} polypeptide of SEQ ID NO:226 and the V_{L} polypeptide of SEQ ID NO:228;
(xiv) one comprising the V_{H} polypeptide of SEQ ID NO:236 and the V_{L} polypeptide of SEQ ID NO:238;
(xv) one comprising the V_{H} polypeptide of SEQ ID NO:246 and the V_{L} polypeptide of SEQ ID NO:248;
(xvi) one comprising the V_{H} polypeptide of SEQ ID NO:256 and the V_{L} polypeptide of SEQ ID NO:258;
(xvii) one comprising the V_{H} polypeptide of SEQ ID NO:266 and the V_{L} polypeptide of SEQ ID NO:268;
(xviii) one comprising the V_{H} polypeptide of SEQ ID NO:276 and the V_{L} polypeptide of SEQ ID NO:278;
(xix) one comprising the V_{H} polypeptide of SEQ ID NO:286 and the V_{L} polypeptide of SEQ ID NO:288;
(xx) one comprising the V_{H} polypeptide of SEQ ID NO:296 and the V_{L} polypeptide of SEQ ID NO:298;
(xxi) one comprising the V_{H} polypeptide of SEQ ID NO:306 and the V_{L} polypeptide of SEQ ID NO:308;
(xxii) one comprising the V_{H} polypeptide of SEQ ID NO:316 and the V_{L} polypeptide of SEQ ID NO:318;
(xxiii) one comprising the V_{H} polypeptide of SEQ ID NO:326 and the V_{L} polypeptide of SEQ ID NO:328;
(xxiv) one comprising the V_{H} polypeptide of SEQ ID NO:336 and the V_{L} polypeptide of SEQ ID NO:338;
(xxv) one comprising the V_{H} polypeptide of SEQ ID NO:346 and the V_{L} polypeptide of SEQ ID NO:348;
(xxvi) one comprising the V_{H} polypeptide of SEQ ID NO:356 and the V_{L} polypeptide of SEQ ID NO:358;
(xxvii) one comprising the V_{H} polypeptide of SEQ ID NO:366 and the V_{L} polypeptide of SEQ ID NO:368;
(xxviii) one comprising the V_{H} polypeptide of SEQ ID NO:376 and the V_{L} polypeptide of SEQ ID NO:378;
(xxix) one comprising the V_{H} polypeptide of SEQ ID NO:386 and the V_{L} polypeptide of SEQ ID NO:388;
(xxx) one comprising the V_{H} polypeptide of SEQ ID NO:396 and the V_{L} polypeptide of SEQ ID NO:398;
(xxxi) one comprising the V_{H} polypeptide of SEQ ID NO:406 and the V_{L} polypeptide of SEQ ID NO:408;
(xxxii) one comprising the V_{H} polypeptide of SEQ ID NO:416 and the V_{L} polypeptide of SEQ ID NO:418;
(xxxiii) one comprising the V_{H} polypeptide of SEQ ID NO:426 and the V_{L} polypeptide of SEQ ID NO:428;
(xxxiv) one comprising the V_{H} polypeptide of SEQ ID NO:436 and the V_{L} polypeptide of SEQ ID NO:438;
(xxxv) one comprising the V_{H} polypeptide of SEQ ID NO:446 and the V_{L} polypeptide of SEQ ID NO:448;
(xxxvi) one comprising the V_{H} polypeptide of SEQ ID NO:456 and the V_{L} polypeptide of SEQ ID NO:458;
(xxxvii) one comprising the V_{H} polypeptide of SEQ ID NO:466 and the V_{L} polypeptide of SEQ ID NO:468;
(xxxviii) one comprising the V_{H} polypeptide of SEQ ID NO:476 and the V_{L} polypeptide of SEQ ID NO:478;
(xxxix) one comprising the V_{H} polypeptide of SEQ ID NO:486 and the V_{L} polypeptide of SEQ ID NO:488;
(xl) one comprising the V_{H} polypeptide of SEQ ID NO:496 and the V_{L} polypeptide of SEQ ID NO:498;
(xli) one comprising the V_{H} polypeptide of SEQ ID NO:506 and the V_{L} polypeptide of SEQ ID NO:508;
(xlii) one comprising the V_{H} polypeptide of SEQ ID NO:516 and the V_{L} polypeptide of SEQ ID NO:518;
(xliii) one comprising the V_{H} polypeptide of SEQ ID NO:526 and the V_{L} polypeptide of SEQ ID NO:528;
(xliv) one comprising the V_{H} polypeptide of SEQ ID NO:536 and the V_{L} polypeptide of SEQ ID NO:533, 534 and 535;
(xlv) one comprising the V_{H} polypeptide of SEQ ID NO:546 and the V_{L} polypeptide of SEQ ID NO:548;
(xlvi) one comprising the V_{H} polypeptide of SEQ ID NO:556 and the V_{L} polypeptide of SEQ ID NO:558;
(xlvii) one comprising the V_{H} polypeptide of SEQ ID NO:566 and the V_{L} polypeptide of SEQ ID NO:568;
(xlviii) one comprising the V_{H} polypeptide of SEQ ID NO:576 and the V_{L} polypeptide of SEQ ID NO:578;
(xlix) one comprising the V_{H} polypeptide of SEQ ID NO:586 and the V_{L} polypeptide of SEQ ID NO:588;
(I) one comprising the V_{H} polypeptide of SEQ ID NO:596 and the V_{L} polypeptide of SEQ ID NO:598;
(li) one comprising the V_{H} polypeptide of SEQ ID NO:606 and the V_{L} polypeptide of SEQ ID NO:608;
(lii) one comprising the V_{H} polypeptide of SEQ ID NO:616 and the V_{L} polypeptide of SEQ ID NO:618;
(liii) one comprising the V_{H} polypeptide of SEQ ID NO:626 and the V_{L} polypeptide of SEQ ID NO:628;
(liv) one comprising the V_{H} polypeptide of SEQ ID NO:636 and the V_{L} polypeptide of SEQ ID NO:638;
(lv) one comprising the V_{H} polypeptide of SEQ ID NO:646 and the V_{L} polypeptide of SEQ ID NO:648;
(lvi) one comprising the V_{H} polypeptide of SEQ ID NO:656 and the V_{L} polypeptide of SEQ ID NO:658;
(lvii) one comprising the V_{H} polypeptide of SEQ ID NO:666 and the V_{L} polypeptide of SEQ ID NO:668;
(lviii) one comprising the V_{H} polypeptide of SEQ ID NO: 676 and the V_{L} polypeptide of SEQ ID NO:678;
(lix) one comprising the V_{H} polypeptide of SEQ ID NO:686 and the V_{L} polypeptide of SEQ ID NO:688;
(lx) one comprising the V_{H} polypeptide of SEQ ID NO: 696 and the V_{L} polypeptide of SEQ ID NO:698;
(lxi) one comprising the V_{H} polypeptide of SEQ ID NO:706 and the V_{L} polypeptide of SEQ ID NO:708;
(lxii) one comprising the V_{H} polypeptide of SEQ ID NO:716 and the V_{L} polypeptide of SEQ ID NO:718;
(lxiii) one comprising the V_{H} polypeptide of SEQ ID NO:726 and the V_{L} polypeptide of SEQ ID NO:728;
(lxiv) one comprising the V_{H} polypeptide of SEQ ID NO:736 and the V_{L} polypeptide of SEQ ID NO:738;
(lxv) one comprising the V_{H} polypeptide of SEQ ID NO:746 and the V_{L} polypeptide of SEQ ID NO:748;
(lxvi) one comprising the V_{H} polypeptide of SEQ ID NO:756 and the V_{L} polypeptide of SEQ ID NO:758;
(lxvii) one comprising the V_{H} polypeptide of SEQ ID NO:766 and the V_{L} polypeptide of SEQ ID NO:768;
(lxviii) one comprising the V_{H} polypeptide of SEQ ID NO:776 and the V_{L} polypeptide of SEQ ID NO:778;
(lxix) one comprising the V_{H} polypeptide of SEQ ID NO:786 and the V_{L} polypeptide of SEQ ID NO:788;
(lxx) one comprising the V_{H} polypeptide of SEQ ID NO:796 and the V_{L} polypeptide of SEQ ID NO:798;
(lxxi) one comprising the V_{H} polypeptide of SEQ ID NO:806 and the V_{L} polypeptide of SEQ ID NO:808; and (lxxii) one comprising the V_{H} polypeptide of SEQ ID NO:816 and the V_{L} polypeptide of SEQ ID NO: 818.

It is a specific object of the invention to provide a use, medicament, composition or method according to any of the foregoing, wherein the ADC comprises an anti-human VISTA antibody or antibody fragment, wherein the anti-VISTA antibody or antibody fragment comprises the same variable regions as one of VSTB92, VSTB56, VSTB95, VSTB103 and VSTB66.

It is a specific object of the invention to provide the use, medicament, composition or method according to any of the foregoing, wherein the ADC comprises an anti-human VISTA antibody or antibody fragment, optionally having CDR or variable sequences of one in FIG. 8, 10 or 12, wherein the anti-VISTA antibody or antibody fragment comprises a human IgG2 kappa backbone with V234A/G237A/P238S/H268A/V309L/A330S/P331S silencing mutations in the Fc region.

It is a specific object of the invention to provide the use, medicament, composition or method according to any of the foregoing, wherein the ADC comprises an anti-human VISTA antibody or antibody fragment, optionally having CDR or variable sequences of one in FIG. 8, 10 or 12, wherein the anti-VISTA antibody or antibody fragment comprises a human IgG1/kappa backbone with L234A/L235A silencing mutations in the Fc region.

It is a specific object of the invention to provide an ADC according to any of the foregoing wherein the glucocorticosteroid agonist or linker conjugate is conjugated to an antibody or antibody fragment that specifically binds to a human immune cell expressed antigen, e.g., an anti-VISTA antibody or antigen binding fragment via its interchain disulfides.

It is a specific object of the invention to provide a pharmaceutical composition comprising a therapeutically effective amount of at least one antibody drug conjugate (ADC) or steroid agonist or steroid-linker according to any of the foregoing and a pharmaceutically acceptable carrier.

It is a specific object of the invention to provide a composition according to any of the foregoing, which is administrable via an injection route, optionally intravenous, intramuscular, intrathecal, or subcutaneous administration.

It is a specific object of the invention to provide a composition according to any of the foregoing, which is subcutaneously administrable.

It is a specific object of the invention to provide a device comprising the glucocorticosteroid agonist, linker conjugate, ADC, composition or medicament according to any of the foregoing, that provides for subcutaneous administration selected from the group consisting of a syringe, an injection device, an infusion pump, an injector pen, a needleless device, an autoinjector, and a subcutaneous patch delivery system.

It is a specific object of the invention to provide a device as set forth above, which delivers to a patient a fixed dose of the glucocorticoid receptor agonist, or a functional derivative thereof, optionally which further comprises instructions informing the patient how to administer the ADC composition comprised therein and the dosing regimen.

It is a specific object of the invention to provide a method of treatment and/or prophylaxis, comprising administering to a patient in need thereof at least one antibody drug conjugate (ADC) or steroid or composition according to any of the foregoing, wherein said composition may be in a device according to any of the foregoing.

It is a specific object of the invention to provide a method of treatment and/or prophylaxis according to any of the foregoing, which is used in the treatment of allergy, autoimmunity, transplant, gene therapy, inflammation, GVHD or sepsis, or to treat or prevent inflammatory, autoimmune, or allergic side effects associated with any of the foregoing conditions in a human subject, optionally wherein the inflammation is associated with cancer, or an infection, optionally a viral or bacterial infection.

It is a specific object of the invention to provide a method of treatment and/or prophylaxis according to any of the foregoing, wherein the patient comprises a condition selected from rheumatoid arthritis, juvenile idiopathic arthritis, psoriatic arthritis, ankylosing spondylitis, adult Crohn's disease, pediatric Crohn's disease, ulcerative colitis, plaque psoriasis, hidradenitis suppurativa, uveitis, Bechet's disease, a spondyloarthropathy, or psoriasis.

It is a specific object of the invention to provide a method of treatment and/or prophylaxis according to any of the foregoing, wherein the patient comprises one or more of the following:
(i) a chronic, acute, episodic allergic, inflammatory or inflammatory condition, e.g., chronic, acute, episodic, remitting/relapsing;
(ii) a condition primarily only effectively treatable with high doses of steroids, optionally polymyalgia rheumatica and/or giant cell arteritis, which patient optionally has been treated or is undergoing treatment with high steroid doses;
(iii) a condition with a comorbidity limiting steroid use, optionally diabetes mellitis, nonalcoholic steatohepatitis (NASH), morbid obesity avascular necrosis/osteonecrosis (AVN), glaucoma, steroid-induced hypertension, severe skin fragility, and/or osteoarthritis;
(iv) a condition wherein safe long-term treatment agents are available, but wherein several months of induction with high-doses of steroids is desired, optionally AAV, polymyositis, dermamyositis, lupus, inflammatory lung disease, autoimmune hepatitis, inflammatory bowel disease, immune thrombocytopenia, autoimmune hemolytic anemia, gout patients wherein several months of induction with high-doses of steroids is therapeutically warranted;
(v) dermatologic conditions that require short/long-term treatment, optionally of uncertain treatment or duration and/or no effective alternative to steroid administration, optionally Stevens Johnson, other severe drug eruption conditions, conditions involving extensive contact dermatitis, other severe immune-related dermatological conditions such as PG, LCV, Erythroderma and the like;
(vi) conditions treated with high-dose corticosteroids for flares/reoccurrences, optionally COPD, asthma, lupus, gout, pseudogout;
(vii) immune-related neurologic diseases such as small-fiber neuropathy, MS (subset), chronic inflammatory demyelinating polyneuropathy, myasthenia gravis and the like;
(viii) hematological/oncology indications, optionally wherein high doses of steroids would potentially be therapeutically warranted or beneficial;
(ix) ophthalmologic conditions, optionally uveitis, iritis, scleritis, and the like;
(x) conditions associated with permanent or very prolonged adrenal insufficiency or secondary adrenal insufficiency, optionally latrogenic Addisonian crisis;
(xi) conditions often treated with long term, low dose steroids, optionally lupus, RA, psA, vasculitis, and the like;
(xii) special classes of patients such as pregnant/breast-feeding women, pediatric patients optionally those with growth impairment or cataracts, or any combination of the foregoing.

It is an object of the invention to provide a method, medicament or use according to any of the foregoing, wherein the patient is further being treated with another active agent.

It is a specific object of the invention to provide a method of treatment and/or prophylaxis according to any of the foregoing, wherein the patient is further being treated with an immunomodulatory antibody or fusion protein which is optionally selected from immmunoinhibitory antibodies or fusion proteins targeting one or more of CTLA4, PD-1, PDL-1, LAG-3, TIM-3, BTLA, B7-H4, B7-H3, VISTA, and/or agonistic antibodies or fusion protein targeting one or more of CD40, CD137, OX40, GITR, CD27, CD28 or ICOS.

It is a specific object of the invention to provide ex vivo use of an ADC or steroid according to any one of the foregoing, wherein immune cells from a patient or donor are contacted with an ADC or steroid according to any one of the foregoing, and then infused into a patient in need thereof, e.g., one with one or more of the conditions identified previously.

It is a specific object of the invention to provide an ADC according to any of the foregoing, wherein the linker is a positive, negative or neutral charged cleavable peptide, optionally esterase cleavable.

It is a specific object of the invention to provide an ADC of any of the foregoing, wherein the drug antibody ratio ranges from about 1:1-12:1 or 1:1-10:1.

It is a specific object of the invention to provide an ADC according to any of the foregoing, wherein the drug antibody ratio ranges from about 2-8:1, 4-8:1, or 6-8:1.

It is a specific object of the invention to provide an ADC according to any of the foregoing, wherein the drug antibody ratio the drug antibody ratio is about 8:1 (n=8) or 4:1 (n=4).

It is a specific object of the invention to provide an ADC according to any of the foregoing, which internalizes one or more of activated or non-activated monocytes, myeloid cells, B cells, NK cells, T cells, CD4 T cells, CD8 T cells, Tregs, mast cells, eosinophils, dendritic cells, mast cells, macrophages and neutrophils, among other immune cell types.

It is a specific object of the invention to provide an ADC according to any of the foregoing, which does not appreciably internalize activated or non-activated B cells.

It is a specific object of the invention to provide an ADC according to any of the foregoing, when administered to a subject in need thereof promotes the efficacy and/or reduces adverse side effects associated with the glucocorticoid receptor agonist, compared to the same dosage of anti-inflammatory agent administered in naked (non-conjugated) form.

It is a specific object of the invention to provide an ADC according to any of the foregoing, wherein the glucocorticoid receptor agonist is conjugated to the antibody or antigen-binding fragment via the interchain disulfides.

It is a specific object of the invention to provide an ADC according to any of the foregoing, which comprises an esterase sensitive linker.

It is a specific object of the invention to provide an ADC according to any of the foregoing, comprising a cleavable linker susceptible to one or more of acid-induced cleavage, photo-induced cleavage, peptidase-induced cleavage, esterase-induced cleavage, and disulfide bond cleavage.

It is a specific object of the invention to provide an ADC according to any of the foregoing, which comprises a non-cleavable linker that is substantially resistant to one or more of acid-induced cleavage, photo-induced cleavage, peptidase-induced cleavage, esterase-induced cleavage and disulfide bond cleavage.

It is a specific object of the invention to provide an ADC according to any of the foregoing, wherein the anti-VISTA antigen binding fragment comprised in the ADC comprises a Fab, F(ab′)2, or scFv antibody fragment.

It is a specific object of the invention to provide a method of treatment and/or prophylaxis, comprising administering to a patient in need thereof at least one antibody drug conjugate (ADC) or composition wherein said composition may be in a device according to any of the foregoing.

It is a specific object of the invention to provide a method of treatment and/or prophylaxis, comprising administering to a patient in need thereof at least one antibody drug conjugate (ADC) or composition wherein said composition may be in a device according to any of the foregoing for the treatment of allergy, autoimmunity, transplant, gene therapy, inflammation, GVHD or sepsis, or to treat or prevent inflammatory, autoimmune, or allergic side effects associated with any of the foregoing conditions in a human subject.

It is a specific object of the invention to provide a method of treatment and/or prophylaxis, comprising administering to a patient in need thereof at least one antibody drug conjugate (ADC) or composition wherein said composition may be in a device according to any of the foregoing for the treatment of patient who comprises a condition selected from rheumatoid arthritis, juvenile idiopathic arthritis, psoriatic arthritis, ankylosing spondylitis, adult Crohn's disease, pediatric Crohn's disease, ulcerative colitis, plaque psoriasis, hidradenitis suppurativa, uveitis, Bechet's disease, a spondyloarthropathy, or psoriasis.

It is a specific object of the invention to provide a method of treatment and/or prophylaxis, comprising administering to a patient in need thereof at least one antibody drug conjugate (ADC) or composition wherein said composition may be in a device according to any of the foregoing for the treatment of patient who comprises one or more of the following:
(i) a condition primarily only effectively treatable with high doses of steroids, optionally polymyalgia rheumatica and/or giant cell arteritis, which patient optionally has been treated or is undergoing treatment with high steroid doses;
(ii) a condition with a comorbidity limiting steroid use, optionally diabetes mellitis, nonalcoholic steatohepatitis (NASH), morbid obesity avascular necrosis/osteonecrosis (AVN), glaucoma. Steroid-induced hypertension, severe skin fragility, and/or osteoarthritis;
(iii) a condition wherein safe long-term treatment agents are available, but wherein several months of induction with high-doses of steroids is desired, optionally AAV, polymyositis, dermamyositis, lupus, inflammatory lung disease, autoimmune hepatitis, inflammatory bowel disease, immune thrombocytopenia, autoimmune hemolytic anemia, gout patients wherein several months of induction with high-doses of steroids is therapeutically warranted;
(iv) dermatologic conditions that require short/long-term treatment, optionally of uncertain treatment or duration and/or no effective alternative to steroid administration, optionally Stevens Johnson, other severe drug eruption conditions, conditions involving extensive contact dermatitis, other severe immune-related dermatological conditions such as PG, LCV, Erythroderma and the like;
(v) conditions treated with high-dose corticosteroids for flares/reoccurrences, optionally COPD, asthma, lupus, gout, pseudogout;
(vi) immune-related neurologic diseases such as small-fiber neuropathy, MS (subset), chronic inflammatory demyelinating polyneuropathy, myasthenia gravis and the like;
(vii) hematological/oncology indications, optionally wherein high doses of steroids would potentially be therapeutically warranted or beneficial;
(viii) ophthalmologic conditions, optionally uveitis, iritis, scleritis, and the like;
(ix) conditions associated with permanent or very prolonged adrenal insufficiency or secondary adrenal insufficiency, optionally latrogenic Addisonian crisis;
(x) conditions often treated with long term, low dose steroids, optionally lupus, RA, psA, vasculitis, and the like; and
(xi) special classes of patients such as pregnant/breast-feeding women, pediatric patients optionally those with growth impairment or cataracts.

It is a specific object of the invention to provide a method of treatment and/or prophylaxis, comprising administering to a patient in need thereof at least one antibody drug conjugate (ADC) or composition wherein said composition may be in a device according to any of the foregoing for the treatment of patient who is further being treated with another active agent.

It is a specific object of the invention to provide a method of treatment and/or prophylaxis, comprising administering to a patient in need thereof at least one antibody drug conjugate (ADC) or composition wherein said composition may be in a device according to any of the foregoing for the treatment of patient who is further being treated with an immunomodulatory antibody or fusion protein which is optionally selected from immmunoinhibitory antibodies or fusion proteins targeting one or more of CTLA4, PD-1, PDL-1, LAG-3, TIM-3, BTLA, B7-H4, B7-H3, VISTA, and/or agonistic antibodies or fusion protein targeting one or more of CD40, CD137, OX40, GITR, CD27, CD28 or ICOS.

It is a specific object of the invention to provide a method of treatment and/or prophylaxis, comprising administering to a patient in need thereof at least one antibody drug conjugate (ADC) or composition wherein said composition may be in a device according to any of the foregoing for the treatment or prophylaxis of acute or chronic inflammation and autoimmune and inflammatory indications associated therewith wherein the conditions optionally include severe asthma, giant cell arteritis, ANKA vasculitis and IBD (Colitis and Crohns).

It is a specific object of the invention to provide a method of treatment and/or prophylaxis, comprising administering to a patient in need thereof at least one antibody drug conjugate (ADC) or composition wherein said composition may be in a device according to any of the foregoing for the treatment or prophylaxis of a condition selected from rheumatoid arthritis, juvenile idiopathic arthritis, psoriatic arthritis, ankylosing spondylitis, adult Crohn's disease, pediatric Crohn's disease, ulcerative colitis, plaque psoriasis, hidradenitis suppurativa, uveitis, Bechet's disease, a spondyloarthropathy, or psoriasis.

It is a specific object of the invention to provide a method for effecting internalization of a steroid into one or more of myeloid cells, T cells, CD4 T cells, CD8 T cells, Tregs, NK cells, neutrophils, monocytes, B cells, NK cells, myeloid cells, dendritic cells, eosinophils, mast cells, and macrophages, among other immune cell types, comprising administering to a subject or contacting said cells ex vivo with an ADC according to any of the foregoing.

It is a specific object of the invention to provide a method for effecting internalization of a steroid into one or more of myeloid cells, NK cells, B cells, T cells, CD4 T cells, CD8 T cells, Tregs, NK cells, Neutrophils, monocytes, myeloid cells, Dendritic cells, eosinophils, mast cells, and macrophages comprising administering to a subject or contacting said cells ex vivo with an ADC according to any of the foregoing, which is effected ex vivo, and a purified or enriched composition comprising immune cells or comprising a specific type or types of immune cells selected from cells, CD4 T cells, CD8 T cells, Tregs, NK cells, Neutrophils, monocytes, myeloid cells, mast cells, Dendritic cells, eosinophils, and macrophages, among other immune cell types is contacted ex vivo with an ADC according to any of the foregoing and afterward introduced into a patient in need thereof.

It is a specific object of the invention to provide a method for effecting internalization of a steroid into one or more of myeloid cells, NK cells, B cells, T cells, CD4 T cells, CD8 T cells, Tregs, NK cells, Neutrophils, monocytes, mast cells, myeloid cells, Dendritic cells, eosinophils, mast cells, and macrophages comprising administering to a subject or contacting said cells ex vivo with an ADC according to any of the foregoing method for treating an inflammatory or autoimmune or allergic condition involving one or more of any of myeloid cells, NK cells, B cells, T cells, Tregs, NK cells, Neutrophils, monocytes, myeloid cells, Dendritic cells, mast cells, eosinophils, and macrophages comprising administering to a subject in need thereof an ADC according to any of the foregoing.

### BRIEF DESCRIPTION OF THE FIGURES

FIG. 1A-B: This Figure shows peptide mapping of a control VISTA antibody 767-IgG1.3 by trypsin digestion. The determined sequence for 767-IgG1.3 with identified tryptic peptides is underlined (A) Light chain (85.6% coverage) (B) Heavy chain (76.1% coverage).

FIG. 2A-B: This Figure shows the determined sequence for 767-IgG1.3 using Lys-C digestion. In the Figure Lys-C peptides are underlined (A) Light chain (63.3% coverage) (B) Heavy chain (76.3% coverage).

FIG. 3: This Figure contains the results of a binding experiment confirming that the synthesized control antibody 767-IgG1.3 and INX200 exhibit opposite pH dependent binding characteristics.

FIG. 4A-C: This Figure contains the results of binding studies revealing that DAR 8 conjugation with linker A does not impact VISTA binding to (A) INX200 (B) INX201 or (C) 767-IgG1.3.

FIG. 5: This Figure contains the results of a ConA experiment wherein female hVISTA knock-in animals were administered different naked and Dex conjugated anti-VISTA antibodies which detected G-CSF changes 6h post ConA in peripheral blood. Plasma concentrations measured using a mouse 7-plex (SEM; n=5/group) (Dosing: Dex-0.2=0.2 mg/Kg, Dex-2=2 mg/Kg, INX210 and INX210A at 10 mg/Kg, [INX210A provided 0.2 mg/kg dex payload]).

FIG. 6: This Figure contains the results of the results of a ConA studies wherein male hVISTA knock-in animals were administered different naked and Dex conjugated anti-VISTA antibodies. In the experiments in the Figure cytokine changes 6h post ConA in peripheral blood. Plasma concentrations measured using a mouse 7-plex (SEM; n=10/group, ordinary one-way ANOVA as compared to ConA-only group) (Dosing: Dex at 0.2 or 5 mg/Kg, INX210 and INX210A at 10 mg/Kg).

FIG. 7: This Figure contains the results of the results of a ConA experiment wherein animals were administered different naked and Dex conjugated anti-VISTA antibodies and cytokine changes were detected 6h post ConA in peripheral blood. Plasma concentrations were measured using an ELISA assay (SD; n=6/group; one-way ANOVA as compared to ConA-only group) (Dosing: Dex at 0.02, 0.2 or 2 mg/Kg, INX200A at 10, 5 and 1 mg/Kg).

FIG. 8 contains the sequences and sequence legend for the variable heavy and light and constant regions of INX200, INX201 and INX210.

FIG. 9 depicts exemplary budenoside derivatives.

FIG. 10A-10JJ contain a sequence table containing the CDR, variable heavy and light sequences, framework sequences and constant domains of exemplary anti-human VISTA antibodies VSTB49-VSTB116 (which possess short serum half-life in rodents and primates at physiological conditions (pH≈7.5)) and epitope information.

FIGS. 11A-11C contain exemplary steroid structures from those disclosed in Example 3.

FIGS. 12A-C contain the sequences of exemplary anti-VISTA antibodies and control antibodies disclosed in the Examples.

FIG. 13 contains a binding study for INX200, and 767-IgG1.3 vs. human IgG1si. Median fluorescence intensity measured for monocytes incubated with serial dilutions of antibodies tested (0-333 nM); dashed black line corresponds to autofluorescence of unstained cells; n=1.

FIG. 14 depicts what fraction of anti-VISTA antibodies (INX200) is internalized by immune cells. The intracellular pool of the cell bound antibodies were plotted over the 60 min of the time course; for each data point fluorescence was normalized to fluorescence of INX200 at time 0 min; mean±SD n=2 donors.

FIG. 15 contains the results of experiments assessing the internalization rate of the INX200 antibody. The internalization rate of INX200 antibody was assessed in monocytes over 60 min time course; anti-CD45 antibody was not internalized at any timepoint; shown as mean±SD, n=2 donors.

FIG. 16: contains a PK study for INX200, INX200A vs. human IgG1. Plasma concentrations of antibodies at annotated time points in hVISTA KI mice (SD; n=5/group).

FIG. 17: contains a PK study for 767-IgG1.3, 767-IgG1.3A vs. human IgG1. Plasma concentrations of antibodies at annotated time points in hVISTA KI mice (SD; n=5/group).

FIG. 18 contains the results of experiments assessing the potency of ADC conjugates according to the invention. In the experiments FKBP5 transcriptional activation following Dex (left) and ADC INX201J (right) treatment in peritoneal resident macrophages and spleen monocytes was assessed. Dex (left) effects were evaluated at 4 and 24h post 1 single i.p. injection at 2 mg/Kg. ADC (right) effects were analyzed at 24, 48, 72 and 96h post 1 single i.p. injection at 10 mg/Kg delivering 0.2 mg/Kg of GC payload. FKBP5 transcription levels were measured by real time PCR and presented as Log 2 fold change vs. PBS control group. Four mice per group were pooled together to generate sufficient material for the RNA preparation.

FIG. 19 contains the results of in vivo experiments showing that Dex treatment prevents the ex vivo induction of pro-inflammatory cytokines in PRM. Dex effects were evaluated at 2h post 1 single i.p. injection at 2 mg/Kg; IL-6 and TNFα were evaluated on cell supernatant (collected at 1h) using a mouse 32-plex (n=4 mice/group; unpaired T test).

FIG. 20 contains the results of experiments assessing the in vivo effects of INX201J or Dex treatment on TNFα in PRMs. The results show that INX201J or Dex treatment prevents the ex vivo induction of TNFα in PRMs. In these experiments Dex effects were evaluated at 2h post 1 single i.p. injection at 2 and 0.2 mg/Kg; INX201J effects were evaluated at 1 day (d-1), 2 days (d-2) and 4 days (d-4) post injection at 10 mg/Kg (equivalent to 0.2 mg/Kg payload). Cell supernatants were collected at 2h. TNFα was measured using an ELISA (n=4 mice/group; ordinary one-way ANOVA as compared to PBS-only group).

FIG. 21 contains the results of experiments assessing the long-term effects of exemplary ADCs according to the invention. The results show that all tested ADCs elicited long-term impact on the ex vivo induction of TNFα and IL-6 in PRMs. Dex effects were evaluated at 2h post 1 single i.p. injection at 2 mg/Kg; INX201J, INX231J, INX234J and INX240 J effects were evaluated at 4 days (−4) and 7 days (−7) post 1 single i.p. injection at 10 mg/Kg. Cell supernatants were collected at 2h. TNFα and IL-6 were measured using ELISA (n=4 mice/group; ordinary one-way ANOVA as compared to PBS-only group).

FIG. 22 contains the results of experiments assessing the potency of exemplary ADC conjugates according to the invention, i.e., INX231J, INX234J and INX240 J. The results indicate that INX231J, INX234J and INX240 J ADCs have comparable potencies in preventing ex vivo induction of TNFα and IL-6 in PRMs. Dex effects were evaluated at 2h post 1 single i.p. injection at 2 mg/Kg; INX231J, INX234J and INX240 J effects were evaluated at 7 days post 1 single i.p. injection at 10, 3 or 1 mg/Kg (0.2, 0.06 and 0.02 mg/Kg of GC payload). Cell supernatants were collected at 2h. TNFα and IL-6 were measured using ELISA (See methods section) (n=4 mice/group except for the PBS group n=1, for technical reasons; ordinary one-way ANOVA as compared to PBS-only group).

FIG. 23 contains experiments showing that the potencies of INX201J, INX201P, INX231J, INX234J and INX240J ADCs are comparable in preventing ex vivo induction of TNFα and IL-6 in PRM. INX201J, INX201P, INX231J, INX234J, INX240 J and Dex effects were evaluated at 7 days post 1 single i.p. injection; ADCs were dosed at 10 mg/Kg (0.2 mg/Kg of GC payload) and Dex at 2 mg/Kg. Cell supernatants were collected at 2h. TNFα and IL-6 were measured using ELISA (n=4 mice/group except for the PBS and Dex groups with n=3, for technical reasons; ordinary one-way ANOVA as compared to PBS-only group).

FIG. 24: INX201J, INX231P, INX234P and INX240P ADCs have comparable potencies in preventing ex vivo induction of TNFα in PRM. ADCs effects were evaluated at 7 days post 1 single i.p. injection; ADCs were dosed at 10 mg/Kg (0.2 mg/Kg of GC payload). Cell supernatants were collected at 2h. TNFα was measured using ELISA (see methods section) (n=4 mice/group; ordinary one-way ANOVA as compared to PBS-only group, SEM).

FIG. 25: INX231P, INX231R, INX233P and INX234P have comparable potencies in preventing ex vivo induction of TNFα and IL-6 in PRM. ADCs effects were evaluated at 7 days post 1 single i.p. injection; ADCs were dosed at 10 mg/Kg (0.2 mg/Kg of GC payload). Cell supernatants were collected at 24h. TNFα and IL-6 were measured using ELISA (see methods section) (n=4 mice/group; ordinary one-way ANOVA as compared to PBS-only group, SEM).

FIG. 26: Potency evaluation of GC linker payloads INX R, INX O, INX S, INX V and INX W vs INX P conjugated to INX231 in preventing ex vivo induction of TNFα and IL-6 in PRM. ADCs effects were evaluated at 7 days post 1 single i.p. injection; ADCs were dosed at 0.2 mg/Kg of GC payload. Cell supernatants were collected at 24h. TNFα and IL-6 were measured using ELISA (see methods section) (n=4 mice/group; ordinary one-way ANOVA as compared to PBS-only group, SEM).

FIG. 27: Potency evaluation of GC payloads INX231S, INX231V, and INX231W vs INX231P in inducing FKBP5 transcription in PRM. ADCs effects were evaluated at 1, 7 and 14 days post 1 single i.p. injection; ADCs were dosed at 0.2 mg/Kg of GC payload. FKBP5 expression was measured by quantitative real time PCR and presented as Log 2 fold change vs. PBS control group (n=4 mice/group; ordinary one-way ANOVA as compared to PBS-only group, SEM).

FIG. 28: Potency evaluation of GC payloads INX231S, INX231V, and INX231W vs INX231P in preventing ex vivo induction of TNFα and IL-6 in PRM. ADCs effects were evaluated at 1, 7 and 14 days post 1 single i.p. injection; ADCs were dosed at 0.2 mg/Kg of GC payload. Cell supernatants were collected at 24h. TNFα and IL-6 were measured using ELISA (see methods section) (n=4 mice/group; ordinary one-way ANOVA as compared to PBS-only group, SEM).

FIG. 29: Potency evaluation of GC payloads INX234A3, INX234A4, INX234T, INX201L and INX231S in inducing FKBP5 transcription in peritoneal resident macrophages (upper row) and spleen cells (lower row). ADCs effects were evaluated at 1, 7 and 14 days post 1 single i.p. injection; ADCs were dosed at 0.2 mg/Kg of GC payload. FKBP5 expression was measured by quantitative real time PCR and presented as Log 2 fold change vs. PBS control group (n=4 mice/group; ordinary one-way ANOVA as compared to PBS-only group, SEM).

FIG. 30: Potency evaluation of GC payloads INX234A3, INX234A4, INX234T, INX201L and INX231S in preventing ex vivo induction of TNFα and IL-6 in PRM. ADCs effects were evaluated at 1, 7 and 14 days post 1 single i.p. injection; ADCs were dosed at 0.2 mg/Kg of GC payload. Cell supernatants were collected at 24h. TNFα and IL-6 were measured using ELISA (see methods section) (n=4 mice/group; ordinary one-way ANOVA as compared to PBS-only group, SEM).

FIG. 31: Potency evaluation of GC payloads INX234V, INX234A5 and INX234A11 in inducing FKBP5 transcription in peritoneal resident macrophages. ADCs effects were evaluated at 7 and 14 days post 1 single i.p. injection; ADCs were dosed at 0.2 mg/Kg of GC payload. FKBP5 expression was measured by quantitative real time PCR and presented as Log 2 fold change vs. PBS control group (n=4 mice/group; ordinary one-way ANOVA as compared to PBS-only group, SEM).

FIG. 32: Potency evaluation of GC payloads INX234V, INX234A5 and INX234A11 in preventing ex vivo induction of TNFα and IL-6 in PRM. ADCs effects were evaluated at 14 days post 1 single i.p. injection; ADCs TNFα and IL-6 were measured using ELISA (see methods section) (n=4 mice/group; ordinary one-way ANOVA as compared to PBS-only group, SEM).

FIG. 33: Potency evaluation of GC payloads INX234V, INX231A7, INX231A12 and INX231A23 in inducing FKBP5 transcription in peritoneal resident macrophages. ADCs effects were evaluated at 7 and 21 days post 1 single i.p. injection; ADCs were dosed at 0.2 mg/Kg of GC payload, except for INX231A7 that was dosed at 0.08 mg/Kg of payload. FKBP5 expression was measured by quantitative real time PCR and presented as Log 2 fold change vs. PBS control group (n=4 mice/group; ordinary one-way ANOVA as compared to PBS-only group, SEM).

FIG. 34: Potency evaluation of GC payloads INX234V, INX231A7, INX231A12 and INX231A23 in preventing ex vivo induction of TNFα and IL-6 in PRM. ADCs effects were evaluated at 7, 14 and 21 days post 1 single i.p. injection; ADCs were dosed at 0.2 mg/Kg of GC payload. Cell supernatants were collected at 24h. TNFα and IL-6 were measured using ELISA (see methods section) (n=4 mice/group; ordinary one-way ANOVA as compared to PBS-only group, SEM).

FIG. 35: IL-12p40 changes at 2 (left) and 4h (right) post LPS in peripheral blood. Plasma concentrations measured using a mouse multi-plex; Dosing: Dex (square) was dosed 2h before LPS stimulation at 0.02, 0.2, 2 and 5 mg/Kg, INX201J (circle) was dosed 2 or 17h before LPS injection at 10 mg/Kg providing 0.2 mg/Kg of GC. The PBS only group (grey solid triangle) indicates the baseline cytokine level in the absence of stimulation; PBS+LPS (black solid triangle) (SEM; n=5/group except where technical failures are excluded from analysis; ordinary one-way ANOVA as compared to PBS+LPS group).

FIG. 36: Cytokine changes at 2h post LPS in peripheral blood. Plasma concentrations measured using a mouse 5-plex; Dosing: Dex was dosed 2h before LPS stimulation at 0.002, 0.02, 0.2, 2 mg/Kg (square) or at 2 mg/Kg 17h pre LPS (black solid square), INX201J (circle) was dosed 17h before LPS injection at 0.02, 0.06, 0.2 mg/Kg of GC payload. The PBS only group (solid grey triangle) indicates the baseline cytokine level in the absence of stimulation; PBS+LPS (solid black triangle) (SEM; n=5/group, except where technical failures are excluded from analysis; ordinary one-way ANOVA as compared to PBS+LPS group).

FIG. 37 shows TNFα changes at 2h post LPS in peripheral blood. TNFα plasma concentrations were measured using ELISA; Dosing: Dex was dosed 2h before LPS stimulation at 0.2 and 2 mg/Kg (square), INX201J (circle) was dosed 17h before LPS injection at 0.06 and 0.2 mg/Kg of GC payload. The PBS group (solid black triangle) received PBS at 2h pre LPS. IgG1siJ (G1siJ) group (triangle) received human IgG1 silent conjugated to GC at 0.2 mg/Kg of payload 17h pre LPS. (SEM; n=5/group except where technical failures are excluded from analysis; ordinary one-way ANOVA as compared to PBS group).

FIG. 38 shows TNFα changes at 2h post LPS in peripheral blood. TNFα plasma concentrations were measured by ELISA; Dosing: Dex was dosed 2h before LPS stimulation at 0.2 and 2 mg/Kg (square), INX201J (circle) and INX201N (inverted triangle) was dosed 17h before LPS injection at 0.2 mg/Kg of GC payload. The PBS group received PBS at 2h pre LPS (solid black triangle). (SEM; n=5/group except where technical failures are excluded from analysis; ordinary one-way ANOVA as compared to PBS group).

FIG. 39 shows TNFα (left) and IL-12p40 (right) changes at 2h post LPS in peripheral blood. Cytokine plasma concentrations were measured by ELISA; Dosing: PBS (solid circle), INX201J (square), INX231J (triangle), INX234J (lozenge), and INX201P (inverted triangle) were dosed 17h before LPS injection, at 0.2 mg/Kg of GC payload (SEM; n=5/group; ordinary one-way ANOVA as compared to PBS group).

FIG. 40 shows TNFα (left) and IL-12p40 (right) changes at 2h post LPS in peripheral blood. Cytokine plasma concentrations were measured by ELISA; Dosing: PBS (solid triangle), INX201J (circle), INX201O (square) and INX201P (lozenge) were dosed 17h before LPS injection at 0.2 mg/Kg of GC payload (SEM; n=5/group except where technical failures are excluded from analysis; ordinary one-way ANOVA as compared to PBS group).

FIG. 41 shows TNFα (right) and IL-12p40 (left) changes at 2h post LPS in peripheral blood. Cytokine plasma concentrations were measured by ELISA; Dosing: PBS, INX201J (circle), INX201O (square) and INX201P (lozenge) were dosed 17h before LPS injection at 0.2 mg/Kg of GC payload (SEM; n=5/group except where technical failures are excluded from analysis; ordinary one-way ANOVA as compared to PBS group (solid black triangle)).

FIG. 42 shows TNFα (right) and IL-12p40 (left) changes at 2h post LPS in peripheral blood. Cytokine plasma concentrations were measured by ELISA; all ADCs and PBS were dosed 20h before LPS injection, at 0.2 mg/Kg of GC payload (INX231P (square), INX231R (triangle), INX233P (lozenge)) (SEM; n=5/group except where technical failures are excluded from analysis; ordinary one-way ANOVA as compared to PBS group (solid circle)).

FIG. 43 shows TNFα (right) and IL-12p40 (left) changes at 2h post LPS in peripheral blood. Cytokine plasma concentrations were measured by ELISA; all ADCs and PBS were dosed 20h before LPS injection, at 0.2 mg/Kg of GC payload (INX231P (solid square), INX231R (solid triangle), INX201O (solid lozenge), INX231S (circle), INX231V (square), INX231W (triangle)) (SEM; n=4/group except for INX231S where 2 technical failures were excluded from analysis; ordinary one-way ANOVA as compared to PBS group (solid circle) showed non-significant data).

FIG. 44 shows FKBP5 transcriptional activation following ADCs treatment in peritoneal resident 4 days post ADC treatment. ADCs were injected i.p. on day 0 delivering 0.2 mg/Kg of GC payload each; PRM were isolated on day 3. FKBP5 transcription levels were measured by real time PCR and presented as Log 2 fold change vs. PBS control group (SEM, ordinary one-way ANOVA as compared to PBS group, n=4).

FIG. 45: TNFα (right) and IL-12p40 (left) changes at 2h post LPS in peripheral blood. Cytokine plasma concentrations were measured by ELISA; all ADCs and PBS were dosed 20h before LPS injection, at 0.2 mg/Kg of GC payload (SEM; n=5/group except for INX234P, INX234A4 and INX234T for which 1 technical failure per group was recorded; ordinary one-way ANOVA as compared to PBS group).

FIG. 46: TNFα (right) and IL-12p40 (left) changes at 2h post LPS in peripheral blood. Cytokine plasma concentrations were measured by ELISA; all ADCs and PBS were dosed 20h before LPS injection, at 0.2 mg/Kg of GC payload (INX234V (solid square), INX234A5 (solid triangle), INX234A11 (solid lozenge)) (SEM; n=5/group ex; ordinary one-way ANOVA as compared to PBS (solid circle) group).

FIG. 47: TNFα (right) and IL-12p40 (left) changes at 2h post LPS in peripheral blood. Cytokine plasma concentrations were measured by ELISA; all ADCs and PBS were dosed 20h before LPS injection, at 0.2 mg/Kg of GC payload except for INX231A7 which was dosed at 0.08 mg/Kg of payload (INX234V (solid square), INX231A7 (solid triangle), INX231A12 (solid lozenge), INX231A23 (circle), INX234A1 (square), INX234A13 (triangle)) (SEM; n=5/group ex; ordinary one-way ANOVA as compared to PBS group).

FIG. 48: TNFα (right) and IL-12p40 (left) changes at 2h post LPS in peripheral blood. Cytokine plasma concentrations were measured by ELISA; all ADCs and PBS were dosed 20h before LPS injection, at 0.2 mg/Kg of GC payload (SEM; n=5/group except but 1 sample had to be censored in the PBS, INX234P, INX201V groups and 2 in the INX234A9 group for technical reason, ordinary one-way ANOVA as compared to the PBS group.

FIG. 49 contains the results of experiments detecting VISTA expression on different cells. As shown therein VISTA is highly expressed in liver endothelial cells. CD45-CD31+ non-immune endothelial cells isolated from hVISTA knock-in mouse liver and stained with anti-human VISTA (red line, shifted right) or unstained (solid gray).

FIG. 50 contains the results of experiments detecting FKBP5 transcriptional activation following INX201J injection in adrenal gland, brain, liver and spleen. As shown therein INX201J effects were measured at 20h post 1 single i.p. injection at 0.3, 3, 10 mg/Kg (delivering 0.006, 0.06, and 0.2 mg/Kg of payload, respectively). Dex effects were measured 2h post a single i.p. injection at 0.2 or 2 mg/Kg. FKBP5 transcription levels were measured by real time PCR and presented as Log 2 fold change vs. the mean of the PBS control group (n=4 mice/group; ordinary one-way ANOVA as compared to PBS-only group).

FIG. 51: INX-SM-3, INX-SM-4, and INX-SM-1 inhibit IL-1β (left) and IL-6 (right) production. Cytokine levels were measured at 24 hr for human PBMCS incubated with 1 ng/mL LPS and serial dilutions (1000-1 nM) of steroid payloads, with the no treatment control plotted on the log-scale x-axis at <1 nM; n=1 donor, standard deviation plotted from technical duplicates.

FIG. 52: INX-SM-1, INX-SM-3, INX-SM-4 and INX-SM-6 inhibit IL-1β production. Cytokine levels measured at 24 hr for human PBMCS incubated with 1 ng/ml LPS and serial dilutions (1000-1 nM) of steroid payloads, with the no treatment control plotted on the log-scale x-axis at <1 nM; n=1 donor, standard deviation plotted from technical duplicates.

FIG. 53: INX-SM-9, INX-SM-31 and INX-SM-35 inhibit IL-1β (top) and IL-6 (bottom) production. Cytokine levels measured at 24 hr for human PBMCS incubated with 1 ng/mL LPS and serial dilutions (1000-0.2 nM) of steroid payloads, with the no treatment control plotted on the log-scale x-axis at <0.2 nM; n=2 donors-representative donor shown. Standard deviation plotted from technical duplicates.

FIG. 54: INX-SM-32 inhibits IL-1β (top) and IL-6 (bottom) production. Cytokine levels measured at 24 hr for human PBMCS incubated with 1 ng/mL LPS and serial dilutions (500-1 nM) of steroid payloads, with the no treatment control plotted on the log-scale x-axis at <1 nM; n=2. Representative donor shown. Standard deviation plotted from technical duplicates.

FIG. 55: INX-SM-10 shows robust inhibition in IL-1β (top) and IL-6 (bottom) production. INX-SM-33 demonstrated modest inhibition of cytokine production. Cytokine levels measured at 24 hr for human PBMCS incubated with 1 ng/ml LPS and serial dilutions (1000-0.5 nM) of steroid payloads, with the no treatment control plotted on the log-scale x-axis at <0.5 nM; n=1 donor, standard deviation plotted from technical duplicates.

FIG. 56: INX-SM-2 and INX-SM-7 show inhibition in IL-1β. Average Cytokine levels measured at 24 hr for human PBMCS incubated with 1 ng/ml LPS and serial dilutions (1000-0.16 nM) of steroid payloads, with the no treatment control plotted on the log-scale x-axis at <0.16 nM; n=1, standard deviation plotted from technical duplicates.

FIG. 57 shows that halogenation at both C6 and C9, but not C9 alone provides increased potency. Average cytokine levels measured at 24 hr for human PBMCS incubated with 1 ng/mL LPS and serial dilutions (1000-0.16 nM) of steroid payloads, with the no treatment control plotted on the log-scale x-axis at <0.16 nM; n=1, standard deviation plotted from technical duplicates.

FIG. 58: INX P conjugated antibodies directed against other surface targets retain anti-inflammatory effects of anti-VISTA conjugates. Average cytokine levels measured at 24 hr for human PBMC incubated with 1 ng/mL LPS and serial dilutions (8,000-0.26 nM conjugated payload) of glucocorticoid conjugates; n=1, mean of technical duplicates.

FIG. 59: INX-SM-43 shows moderate inhibition of huIL1-β. Average cytokine levels measured at 24 hr for human PBMC incubated with 1 ng/ml LPS and serial dilutions (100-0.032 nM) of glucocorticoid payloads; n=1, mean of technical duplicates.

FIG. 60: INX-SM-44 shows moderate inhibition of huIL1-β. Average cytokine levels measured at 24 hr for human PBMC incubated with 1 ng/mL LPS and serial dilutions (1000-0.32 nM) of glucocorticoid payloads; n=1, mean of technical duplicates.

FIG. 61: INX-SM-25 and INX-SM-3 show robust inhibition in IL-1β production. INX-SM-45 and INX-SM-46 demonstrated more modest inhibition of cytokine production. Cytokine levels measured at 24 hr for human PBMC incubated with 1 ng/mL LPS and serial dilutions (1000-0.5 nM) of steroid payloads, with the no treatment control plotted on the log-scale x-axis at <0.5 nM; n=1 donor, mean of technical duplicates plotted.

FIG. 62: Dramatically increased potency of INX231V over INX231P and INX231J. Average cytokine levels for A. hull-1β B. huIL-6 measured at 24 hr for human PBMC incubated with 1 ng/mL LPS and serial dilutions (1000-0.16 nM) of conjugated steroid linker payloads, with the no treatment control plotted on the log-scale x-axis at <0.16 nM; n=1, standard deviation plotted from technical duplicates. Values above the ULOQ (12,500 pg/mL for IL-1b and 150,000 pg/mL for IL-6) were plotted as the extrapolated value.

FIG. 63: INX231V has substantial potency, and INX231P modest potency over INX231J. Average cytokine levels for IL-1β measured at 24 hr for human PBMC incubated with 1 ng/mL LPS and serial dilutions (1000-0.16 nM) of conjugated steroid linker payloads, with the no treatment control plotted on the log-scale x-axis at <0.16 nM; n=1, mean plotted of technical duplicates

FIG. 64: Enhanced potency of INX231S, INX234T and INX234A3 over INX231J. Average cytokine levels for IL-1β measured at 24 hr for human PBMC incubated with 1 ng/mL LPS and serial dilutions (1000-0.16 nM) of conjugated steroid linker payloads, with the no treatment control plotted on the log-scale x-axis at <0.16 nM; n=1, mean plotted of technical duplicates.

FIG. 65: INX201 may lead to enhanced early potency of conjugates vs INX231. Average cytokine levels for IL-1β measured at 24 hr for human PBMC incubated with 1 ng/ml LPS and serial dilutions (1000-0.16 nM) of conjugated steroid linker payloads, with the no treatment control plotted on the log-scale x-axis at <0.16 nM; n=1, mean plotted of technical duplicates.

FIG. 66: Analogs of INX V are potent relative to INX231J. Average cytokine levels for IL-1β measured at 24 hr for human PBMC incubated with 1 ng/ml LPS and serial dilutions (1000-0.16 nM) of conjugated steroid linker payloads, with the no treatment control plotted on the log-scale x-axis at <0.16 nM; n=1, mean plotted of technical duplicates.

FIG. 67: INX-SM-36, INX-SM-32 (top) and INX-SM-3, INX-SM-J2 (bottom) inhibit IL-1β. INX-SM-32 and INX-SM-36 inhibit IL-1β with similar potency to dexamethasone. INX-SM-3 and INX-SM-J2 have similar potency to budesonide. Cytokine levels measured at 24 hr for human PBMC incubated with 1 ng/mL LPS and serial dilutions (1000-0.5 nM) of steroid payloads, with the no treatment control plotted on the log-scale x-axis at <0.5 nM; n=1 donor, mean of technical duplicates plotted.

FIG. 68: INX-SM-32, INX-J2, and INX-SM-3 elicit similar inhibition of IL-1β, INX-SM-37 weakly inhibits IL-1β. INX-SM-32, INX-J2 and INX-SM-3 inhibit IL-1β with similar potency. Cytokine levels measured at 24 hr for human PBMC incubated with 1 ng/mL LPS and serial dilutions (1000-0.15 nM) of steroid payloads, with the no treatment control plotted on the log-scale x-axis at <0.5 nM; n=1 donor, mean of technical duplicates plotted.

FIG. 69: INX231V is substantially more potent than other INX231/INX234 conjugates. Average cytokine levels for IL-1β measured at 24 hr for human PBMC incubated with 1 ng/mL LPS and serial dilutions (1000-0.16 nM) of conjugated steroid linker payloads, with the no treatment control plotted on the log-scale x-axis at <0.16 nM; n=1, mean plotted of technical duplicates.

FIG. 70: Phosphorylated and halogenated analogs of INX V are potent relative to INX J. Average cytokine levels for IL-1β measured at 24 hr for human PBMC incubated with 1 ng/mL LPS and serial dilutions (1000-0.16 nM) of conjugated steroid linker payloads, with the no treatment control plotted on the log-scale x-axis at <0.16 nM; n=1, mean plotted of technical duplicates.

FIG. 71: INX-SM-14, INX-SM-15 and INX-J2 have similar inhibition of IL-1β (top) and IL-6 (bottom). INX-SM-17 weakly inhibits IL-1β but not IL-6. INX-SM-14, INX-SM-15 and INX-J2 inhibit IL-1β and IL-6 with similar potency. Cytokine levels measured at 24 hr for human PBMC incubated with 1 ng/mL LPS and serial dilutions (1000-0.15 nM) of steroid payloads, with the no treatment control plotted on the log-scale x-axis at 0.01 nM; n=1 donor, mean of technical duplicates plotted

FIG. 72: INX-SM-40 and INX-SM-34 weakly inhibit IL-1β (top) and IL-6 (bottom) relative to INX-J2. Cytokine levels measured at 24 hr for human PBMC incubated with 1 ng/ml LPS and serial dilutions (1000-0.15 nM) of steroid payloads, with the no treatment control plotted on the log-scale x-axis at 0.01 nM; n=1 donor, mean of technical duplicates plotted.

FIG. 73: INX-SM-49 and INX-SM-47 weakly inhibit IL-1β (top) and IL-6 (bottom) relative to INX-J2. Cytokine levels (IL1-β and IL-6) measured at 24 hr for human PBMC incubated with 1 ng/mL LPS and serial dilutions (1000-0.15 nM) of steroid payloads, with the no treatment control plotted on the log-scale x-axis at 0.01 nM; n=1 donor, mean of technical duplicates plotted.

FIG. 74: INX231A9 and INX201V show enhanced potency over INX234J and INX201J in reducing IL-1β production. Cytokine levels measured at 24 hr for human PBMC incubated with 1 ng/mL LPS and serial dilutions of anti-VISTA conjugates with respect to conjugated payload concentration (1000-0.15 nM), with the no treatment control plotted on the log-scale x-axis at 0.1 nM; n=1 donor, mean of technical duplicates plotted.

FIG. 75: INX V and INX A23 at equivalent or reduced DAR show enhanced potency over INX J in reducing IL-1β production. Cytokine levels measured at 24 hr for human PBMC incubated with 1 ng/mL LPS and serial dilutions of anti-VISTA conjugates with respect to total ADC concentration (20-0.003 μg/mL), with the no treatment control plotted on the log-scale x-axis at 0.001 μg/mL; n=1 donor, mean of technical duplicates plotted

FIG. 76: INX V conjugates have enhanced potency over INX J conjugate in IL-1β impact even with reduced DAR. Cytokine levels measured at 24 hr for human PBMC incubated with 1 ng/mL LPS and serial dilutions of anti-VISTA conjugates with respect to total ADC concentration (20-0.003 μg/mL), with the no treatment control plotted on the log-scale x-axis at 0.001 μg/mL; n=1 donor, mean of technical duplicates plotted

FIG. 77 contains the results of experiments comparing the PK properties of an exemplary inventive antibody INX200 vs. human IgG1. As shown therein plasma concentrations of antibodies at annotated time points in hVISTA KI mice (SD; n=5/group).

FIG. 78 contains the results of experiments comparing the PK properties of 767-IgG1.3 vs. human IgG1. As shown therein plasma concentrations of antibodies at annotated time points in hVISTA KI mice (SD; n=5/group).

FIG. 79 contains the results of experiments comparing the PK values of other exemplary anti-VISTA antibodies according to the invention, i.e., INX231, INX234, INX237 and INX240. As shown therein plasma concentrations of antibodies at annotated time points in hVISTA KI mice (SD; n=5/group). Left graph shows y and x axes in Log 10, while for right graph, only the y axis is in Log 10.

FIG. 80 contains the results of experiments comparing the PK values of exemplary anti-VISTA antibodies according to the invention, i.e., INX901, INX904, INX907 and INX908. Plasma concentrations of antibodies at annotated time points in hVISTA KI mice (SD; n=5/group).

FIG. 81 contains the results of experiments comparing the PK values of different ADCs according to the invention, i.e., INX201J, INX231J, INX234J and INX240J. Plasma concentrations of antibodies at annotated time points in hVISTA KI mice (SD; n=4/group).

FIG. 82 contains results of experiments assaying the impact of long-term treatment with an exemplary VISTA Ab ADC conjugate INX201J and dexamethasone on corticosterone levels. The Figure shows changes in plasma corticosterone levels. (SEM, one-way ANOVA, n=8 except for PBS control group in right graph with n=6).

FIG. 83 shows Ag-specific CD8 T cell numbers from peripheral blood on day 6 post immunization in EXPERIMENT 1 in Example 12. (SEM, one-way ANOVA, n=5).

FIG. 84 shows Ag-specific CD8 T cell numbers from peripheral blood on day 6 post immunization in EXPERIMENT 2 in Example 12. The graph on the left shows the PBS control group with all samples included, the one on the right shows the PBS control group with one outlier removed (SEM, one-way ANOVA, n=5 except for naïve; one sample was excluded in the group with Dex at 0.2 mg/Kg as a failed immunization).

FIG. 85 shows Ag-specific CD8 T cell numbers from peripheral blood on day 6 post immunization in Experiment 3 in Example 12. In this experiment, multiple samples had to be excluded due to a technical problem during processing: PBS group n=3, Dex at 2 mg/Kg n=2, Dex at 0.2 mg/Kg n=3, INX201J D-1 n=5, INX201J D-7 n=2, INX231J D-7 n=3, INX234J D-7 n=5, INX240 J D-7 n=4 (SEM, one-way ANOVA, D=day).

FIG. 86 shows Ag-specific CD8 T cell numbers from peripheral blood on day 6 post immunization in Experiment 3 in Example 12. For technical reasons, 2 samples were excluded in the PBS, INX231P and INX234P groups; for all the other groups n=5 (SEM, one-way ANOVA).

FIG. 87 shows changes in absolute cell numbers in peripheral blood in the 2 experiment schedules. OVA challenge on days 14 to 18 and on days 21 to 25 (SEM, one-way ANOVA, n=10 except for naïve group with n=5).

FIG. 88 shows changes in immunoglobulin productions in peripheral blood in the 2 experiment schedules. OVA challenge on days 14 to 18 (Part 1) and on days 21 to 25 (Part 2) (SEM, one-way ANOVA, n=10 except for naïve group with n=5).

FIGS. 89A-89B shows changes in immune infiltrate in BAL in the 2 experiment schedules. OVA challenge on days 14 to 18 (Part 1) and on days 21 to 25 (Part 2); A) Changes in myeloid infiltrate; B) in lymphocytic infiltrate (SEM, one-way ANOVA, n=10 with 2 samples censored in control group, 3 in both Dex group and INX201J group; for naïve group n=5).

FIG. 90 shows changes in cytokine levels in BAL in the 2 experiment schedules. OVA challenge on days 14 to 18 (Part 1) and on days 21 to 25 (Part 2) (SEM, on-way ANOVA, n=10 with 2 samples censored in control group, 3 in both Dex group and INX201J group; for naïve group n=5).

FIG. 91 shows lung disease scoring for Part 1 of the study. (SEM, one-way ANOVA, n=10 except for naïve group n=5).

FIG. 92 shows FKBP5 transcriptional activation following INX231J injection in spleen (left) and blood (right) cells. INX231J effects and hlgG1siJ (grey) were measured at 20h post 1 single i.v. injection at 5 mg/Kg (delivering 0.1 mg/Kg of payload). Dex effects were measured 2h post a single i.p. injection at 2 mg/Kg. FKBP5 transcription levels were measured by real time PCR and presented as Log 2 fold change vs. the mean of the PBS control group. (n=4 mice/group; ordinary one-way ANOVA as compared to PBS-only group.

FIG. 93 shows FKBP5 transcriptional activation following INX231P injection in C57BI/6 mice. INX231P effects were measured at 20h post 1 single i.v. injection at 10 mg/Kg (delivering 0.2 mg/Kg of payload). Dex effects were measured 2h post a single i.p. injection at 2 mg/Kg. FKBP5 transcription levels were measured by real time PCR and presented as Log 2 fold change vs. the mean of the PBS control group. (n=4 mice/group; ordinary one-way ANOVA as compared to PBS-only group).

FIG. 94 contains experimental results which show FKBP5 transcriptional activation following INX231P injection in C57BI/6 or hVISTA KI mice. INX231P effects were measured at 20h post 1 single i.v. injection at 10 mg/Kg (delivering 0.2 mg/Kg of payload). Dex effects were measured 2h post a single i.p. injection at 2 mg/Kg. FKBP5 transcription levels were measured by real time PCR and presented as Log 2 fold change vs. the mean of the PBS control group. (n=4 mice/group; ordinary one-way ANOVA as compared to PBS-only group).

FIG. 95 contains experimental results which show that in vivo Dex treatment causes decrease in ex vivo monocyte inflammatory response to LPS. Mice were injected i.p. with PBS or Dex at 2 mg/Kg or 0.2 mg/Kg. After 2h, spleen monocytes were isolated, put in culture and subjected to LPS stimulation at 0, 10 and 100 ng/ml. 24h supernatants were analyzed on Luminex 32-plex (n=5 mice/group but samples 1,2,3 and 4,5 were pooled into 2 samples).

FIG. 96 contains experiment results which show that in vivo treatment with INX231P impact on ex vivo monocyte inflammatory response to LPS. Mice were injected i.p. with PBS or Dex at 2 mg/Kg 2 h, 2 or 6 days before cell isolation; injected i.v. with INX231P and INX901 at 10 mg/Kg 1, 3 and 7 days before cell isolation. After isolation, spleen monocytes were put in culture and subjected to LPS stimulation at 0 or 10 ng/ml (only 10 ng/ml is shown). 24h supernatants were analyzed by ELISA (n=4 mice/group; one-way ANOVA comparing to PBS treated group was done only for the day 1 (D1) samples).

FIG. 97 contains experiment results which show that in vivo treatment with INX231P impact on ex vivo monocyte inflammatory response to LPS. Mice were injected i.p. with PBS or Dex at 2 mg/Kg 2h before cell isolation; injected i.v. with INX231P and INX901 at 10 mg/Kg 24h before cell isolation. Spleen monocytes were put in culture and subjected to LPS stimulation at 10 and 100 ng/ml. 24h supernatants were analyzed by ELISA (n=4 mice/group; separate ordinary one-way ANOVA as compared to PBS treated group for each LPS dose).

FIG. 98 shows FKBP5 transcriptional activation in B cells or monocytes. Cells were treated with 20 nM of free J payload, or equimolar amounts of payload conjugated to INX201 (INX201J) or isotype control (huIgG1si J). Transcript levels were analyzed as technical duplicates.

FIG. 99 shows FKBP5 transcriptional activation in monocytes. Cells were treated with increasing amount of INX201J [0-100 nM payload]). The 0 payload represents treatment with unconjugated INX201 antibody alone at the same amount of antibody as in the 100 nM payload INX201J dose. Transcript levels were analyzed as technical duplicates.

FIG. 100 shows FKBP induction in T regs from 2 donors treated with 20 nM INX-SM-3 (free payload) or the molar payload equivalent of INX231P (conjugated payload). Samples were generated and analyzed as singlicates. Isolated Treg purity was ≥75% as assessed by flow cytometry.

FIG. 101 shows FKBP5 induction in T regs from 1 donor treated with 20 nM payload equivalent of INX201J relative to 20 nM payload equivalent huIgG1si J. Samples were analyzed as technical duplicates. Isolated Treg purity was ≥75% as assessed by flow cytometry.

FIG. 102 summarizes the reported consensus RNA expression levels by different immune cells for VISTA and other ADC targets (CD40, TNFα, CD74, CD163 (PRLR) based on the “Transcripts Per Million” (TPM) reported wherein a TPM<10 represents (minimal/no expression “−”); a TPM 10-100 represents (low/intermediate expression “+”); and a TPM>100 (high expression “++”).

FIG. 103A-E summarize the quantification of antigen density for VISTA, CD74, CD163 and mTNFα on identified cell populations A) monocytes express VISTA, CD74 and CD163; B) B cells express CD74; C) CD4+ T cells, D) CD4+ T regs and E) CD8+ T cells express VISTA (mean±SD, n=5 donors).

FIGS. 104A-F show the quantification of antigen density for VISTA, CD74, CD163 and mTNFα on identified cell populations in human blood A) monocytes express VISTA, CD74 and CD163; B) B cells express CD74; C) neutrophils express VISTA, D) CD4+ T cells, E) CD4+ T regs and F) CD8+ T cells express VISTA (mean±SD, n=3).

FIG. 105 contains data showing that steroid responsive genes in bone are significantly impacted by free Dex, whereas a VISTA ADC (INX231P) has limited impact. In the figure Fkpb5 is shown on the left, RANKL the middle left, ddit4 the middle right and Fam107a on the extreme right. In the experiments INX231P effects were measured at 20h post 1 single i.p. injection at 10 mg/Kg (delivering 0.2 mg/Kg of payload). Dex effects were measured 2h post a single i.p. injection at 2 mg/Kg. Gene transcription levels were measured by RNAseq and presented as normalized counts. (n=5 mice/group; ordinary one-way ANOVA as compared to PBS-only group).

FIG. 106 contains the results of experiments comparing the effects of a VISTA ADC (INX234P) relative to vehicle control on steroid responsive gene expression in peripheral blood cells in cynomolgus monkeys. In these experiments INX234P effects were measured at 24h post 1 single i.v. dose at 10 mg/Kg (delivering 0.2 mg/Kg of payload). Gene transcription levels were measured by RNAseq and presented as normalized counts. (n=2 vehicle, n=6 ADC/group; unpaired t test vs vehicle).

FIG. 107 contains the results of experiments evaluating the effects of a VISTA ADC (INX234P) on specific non-target cells. As shown from the data INX234P had limited to no impact on FKBP5 in white adipose, brain and bone. In the experiments INX234P effects were measured at 24h post 1 single i.v. dose at 10 mg/Kg (delivering 0.2 mg/Kg of payload) or D8 (vehicle). Gene transcription levels were measured by RNAseq and presented as normalized counts. (n=2 vehicle, n=3 ADC/group for tissues; unpaired t test vs vehicle-INX234P was non-significant).

FIG. 108 contains the results of experiments evaluating the effects of ADCs on steroid responsive genes. The data shows some residual Dex effect at 24h in white adipose tissue; ADC gene expression is similar to vehicle control. Free Dex (2 mg/Kg) and INX234P (10 mg/Kg-delivering 0.2 mg/Kg of payload) effects were measured at 24h post 1 single i.v. dose or day 8 (vehicle). Gene transcription levels were measured by RNAseq and presented as normalized counts. (n=2 vehicle, n=2 dexamethasone, n=3 ADC/group for tissues; unpaired t test vs vehicle).

FIG. 109A-D: contains experimental results showing the high accumulation of active payload (INX-SM-3) at 24h in INX234P treated monkeys correlates with VISTA expressing tissues. Panel (A) shows released payload (INX-SM-3), and Panel (B) shows cysteine modified linker/payload (INXP-cys) and Panel (C) shows dexamethasone, measured at 24h post 1 single i.v. dose of either INX234P (10 mg/kg-delivering 0.2 mg/Kg of payload) or free dexamethasone (2 mg/kg). Panel (D) shows that at day 8, released payload (INX-SM-3) persisted in VISTA expressing tissues in INX234P treated monkeys. Accumulated compound levels were measured by LC-MS/MS and presented as ng of compound/g of tissue (n=3 ADC/group for INX234P except for bone marrow on day 8, n=2 due to limited sample, n=2 Dex) (Duod=duodenum).

FIG. 110 compares the expression of VISTA to other proteins (particularly proteins which have been targeted with other steroid ADCs) on activated immune cells (monocytes). In the experiment human whole blood from healthy donors was activated with LPS (100 μL per well in U bottom 96 well plate; 1 μg/mL LPS; 2 hr at 37° C.). Cell surface protein expression levels on activated immune (monocyte) cells was assessed by flow cytometry. Directly conjugated antibodies used for staining in these experiments included anti-VISTA clone GG8, CD163 clone GHI/61, CD74 clone 332516, and mTNFα clone mAb11. As shown, VISTA expression patterns were similar to expression levels observed on non-activated cells whereas the other protein expression levels were low. Specifically, mTNFα MFI was only marginally higher than the FMO (Fluorescence Minus One) control.

FIG. 111 contains the results of experiments assessing the PK of an ADC according to the invention, i.e., INX234P, in Cyno. INX234P was dosed i.v. at 15 mg/Kg. Animals were bled at the time points indicated, serum was isolated and antibody levels measured (n=4 cynomolgus monkeys, SEM).

FIG. 112 shows hematological changes induced by one dose of INX234P. In these experiments INX234P was dosed i.v. at 15 mg/Kg. Animals were bled at the time points indicated, blood smear done and different cell populations counted (n=4 cynomolgus monkeys, SEM) (WBC=white blood cells, NEUT=neutrophils, LYMPH=lymphocytes, MONO=monocytes, EOS=eosinophils, BASO=basophils, RBC=red blood cells, Retic=reticulocytes).

FIG. 113 contains the results of experiments detecting cortisol changes induced by one dose of INX234P in individual animals. In these experiments INX234P was dosed i.v. at 15 mg/Kg. Animals were bled at the time points indicated, serum isolated and cortisol levels measured by ELISA (n=4 cynomolgus monkeys).

FIG. 114 contains the results of experiments detecting the PK of linker payload (P-cys) and released payload (SM3) in PBL. INX234P was dosed i.v. at 15 mg/Kg. Animals were bled at the time points indicated, PBL isolated and analyzed by MS (n=4 cynomolgus monkeys, SEM).

FIG. 115 contains the results of PK assays which detected the PK of linker payload (P-cys) and released payload (SM3) in serum. INX234P was dosed i.v. at 15 mg/Kg. Animals were bled at the time points indicated, serum isolated and analyzed by MS (n=4 cynomolgus monkeys, SEM).

FIG. 116 contains the results of which show that steroid responsive genes in PBLs are upregulated by INX234P. In these experiments INX234P was dosed i.v. at 15 mg/Kg. Animals were bled at the time points indicated, PBL isolated and subjected to RNA isolation, gene transcription levels were measured by RNAseq and are presented as fold changes vs pre-bleed (n=4 cynomolgus monkeys).

FIG. 117 contains experiments showing the effects of INX201J, INX231P and INX231V on FKBP5 induction in human PBMC by 4h. In the experiments FKBP5 induction was detected in human PBMCs incubated with 1 μM conjugated payload at 4h as measured by RT PCR relative to GAPDH; where n=1 donor.

FIG. 118A-O contains the proprietary and chemical name and structures of exemplary glucocorticoid agonist compounds and glucocorticoid agonist-linker compounds of Formula I, II and III.

FIG. 119 contains the results of GVHD experiments showing that INX234P treatment reduces human PBMC expansion. In these experiments peripheral blood was collected on day 21 and human CD45 positive cells quantified by flow cytometry. Mice were dosed once a week from day 0 to day 34 (SEM; n=8/group) (Dosing at 10 mg/Kg, INX234P provided 0.2 mg/kg of INX P linker payload).

FIG. 120 contains GVHD experimental results showing that INX234P treatment improves mouse survival. Mice were dosed i.p. at 10 mg/Kg (or 0.2 mg/Kg of INX P linker payload) once a week from day 0 to day 34. Upper left graph shows the mean weight changes as percentage of initial body weight; the 3 bottom graphs show the individual mice % of weight loss; the right upper graph is a Kaplan-Meyer survival curve; the grey bar above (lower graphs) or below (upper graphs) indicates the treatment period (SEM; n=8/group).

FIG. 121 contains GVHD experiments showing that INX234V/P treatment reduces human T cell expansion. Peripheral blood was collected weekly starting on day 15 post transfer and human CD45+ CD3+ positive cells were quantified by flow cytometry. Mice were dosed once a week from day 0 (SEM; n=8/group) (Dosing at 10 mg/Kg, INX234V and INX234P provided 0.2 mg/kg of INX V or INX P linker payload respectively).

FIG. 122 contains data obtained in a GVHD model showing that INX234V/P treatment improves mouse survival. Mice were dosed i.p. at 10 mg/Kg (or 0.2 mg/Kg of INX V or INX P linker payload) once a week starting on day 0. Upper left graph shows the mean weight changes as percentage of initial body weight; the 3 bottom graphs show the individual mice % of weight loss; the right upper graph is a Kaplan-Meyer survival curve.

FIG. 123 contains data obtained in a colitis model showing that INX234P treatment improves mouse survival. In the experiments the mice were dosed i.p. at 10 mg/Kg (and 0.2 mg/Kg of INX P linker payload where applicable) with both INX234P and INX234, once a week from day 0 to day 61. The upper left graph shows the mean weight changes as percentage of initial body weight; the 3 bottom graphs show the individual mice % of weight loss; the right upper graph is a Kaplan-Meyer survival curve; the grey bar above (lower graphs) or below (upper graphs) indicates the treatment period (SEM; n=10/group except the INX234P group with n=9).

FIG. 124 contains data obtained in a colitis model showing that high dose dexamethasone treatment improves mouse survival. In the experiments the mice were dosed i.p. at 2 (high) or 0.2 (low) mg/Kg once a week from day 0 to day 61. The upper left graph shows the mean weight changes as percentage of initial body weight; the 3 bottom graphs show the individual mice % of weight loss; the right upper graph is a Kaplan-Meyer survival curve; the grey bar above (lower graphs) or below (upper graphs) indicates the treatment period (SEM; n=10/group).

FIG. 125 contains data obtained in a colitis model showing that INX234V treatment improves mouse survival. In the experiments the mice were dosed i.p. at 10 mg/Kg (and 0.2 mg/Kg of INX V linker payload where applicable) for both INX234V and INX234 once a week from day 21 to day 80. The upper left graph shows the mean weight changes as percentage of initial body weight; the 3 bottom graphs show the individual mice % of weight loss; the right upper graph is a Kaplan-Meyer survival curve; the grey bar above (lower graphs) or below (upper graphs) indicates the treatment period (SEM; n=10/group except for INX234 treated group where n=5).

FIG. 126 contains data obtained in a colitis model showing that low dose dexamethasone treatment improves mouse survival. In the experiments the mice were dosed d i.p. at 2 (high) or 0.2 (low) mg/Kg once a week from day 21 to day 80. The upper left graph shows the mean weight changes as percentage of initial body weight; the 3 bottom graphs show the individual mice % of weight loss; the right upper graph is a Kaplan-Meyer survival curve; the grey bar above (lower graphs) or below (upper graphs) indicates the treatment period (SEM; n=10/group).

FIG. 127 contains data obtained in a colitis model showing that INX234V treatment prevents T cell expansion and activation. In the experiments the mice were dosed i.p. at 10 mg/Kg (and 0.2 mg/Kg of V payload where applicable) for INX234V or INX234 and at 2 (high) or 0.2 (low) mg/Kg of Dex once a week from day 21 to day 80. The upper left graphs show naïve T cell number from blood for INX234V and INX234 vs PBS on the left and Dex at high and low doses vs PBS on the right; lower left and right graphs indicate the frequencies of CD45^{RB+} CD4⁺ T cells for the same groups; the grey bar above indicates the treatment period (SEM; n=10/group except for INX234 treated group where n=5).

### DETAILED DESCRIPTION

Provided herein are novel glucocorticosteroids, glucocorticosteroid-linkers and antibody drug conjugates (ADC's) comprising an antibody or antibody fragment which binds to an antigen expressed on immune cells, typically an antigen expressed on human immune cells. In some embodiments the ADCs comprise an anti-human VISTA (V-region Immunoglobulin-containing Suppressor of T cell Activation (1)) antibody or anti-VISTA antigen-binding antibody fragment, e.g., one having a short serum half-life (˜ 24-27 hours or less in a human VISTA knock-in rodent). In exemplary embodiments the subject ADCs have a rapid onset of action and are potent for prolonged duration as they are very effectively internalized by immune cells in large amounts where they are cleaved releasing large amounts of active steroid payload. The invention also relates to the use of such ADCs and novel steroids for the treatment of autoimmune, allergic and inflammatory conditions. The invention further relates to methods for reducing the adverse side effects and/or enhancing the efficacy of glucocorticoids by using such ADCs to selectively deliver these anti-inflammatory agents to target immune cells, such as monocytes, neutrophils, T cells, Tregs, eosinophils, macrophages, dendritic cells, NK cells, et al., and particularly myeloid cells, thereby reducing potential toxicity otherwise elicited by steroid compounds to non-target cells.

Specifically provided herein are ADC's comprising an anti-VISTA antibody or antibody fragment, typically one which antibody or antibody fragment possesses a very short serum half-life at physiological conditions (pH≈7.5), generally a serum half-life of to 72 hours, 1 to 32 hours, 1 to 16 hours, 1 to 8 hours, 1 to 4 hours or 1-2 hours+0.5 hour in a human VISTA knock-in rodent or ≈3.5, 3, 2.5, or 2.3 days±0.5 days in a primate (Cynomolgus macaque) at physiological conditions (≈pH 7.5) and a glucocorticoid receptor agonist of any one of formulae (I), (II) or (III) disclosed herein, which optionally are attached via a linker, e.g., a peptide or non-peptide linker which optionally may be cleavable under specific conditions, e.g., esterase cleavable dipeptide linker, and which optionally is directly or indirectly attached to an antibody via a heterobifunctional or heterotrifunctional group, wherein such ADC's when administered to a subject in need thereof deliver such glucocorticoid receptor agonist to target immune cells, e.g., monocytes, T cells, neutrophils, Tregs, CD8 T cells, CD4T cells, eosinophils, dendritic cells, NK cells, macrophages, or myeloid cells and result in the functional internalization of the glucocorticoid receptor agonist therein where it elicits the desired inhibitory effect on inflammation without eliciting or eliciting substantially reduced adverse side effects such as toxicity to non-target cells. Further provided are methods of making such ADCs and methods of using the same, in particular for use in the treatment of autoimmune, allergic and inflammatory conditions such as those previously identified.

More specifically provided are glucocorticoid agonist compound having the following structure of Formula (I):

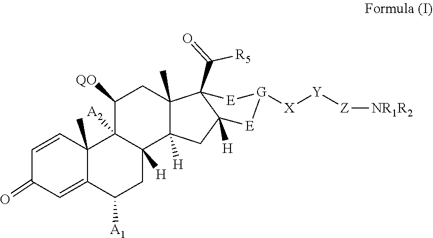

X is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo[2.2.2]octane, adamantane, and cubane each of which can be substituted with 1-4 heteroatoms independently selected from F, Cl, Br, I, N, S, and O, each of which ring structure may contain at least one skeletal heteroatom selected from N, S, and O, and are optionally further substituted with 1-4 C1-3 alkyl or C1-3 perfluoroalkyl;
Z is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo[2.2.2]octane, adamantane, and cubane each of which can be substituted with 1-4 heteroatoms independently selected from F, Cl, Br, I, N, S, and O, each of which ring structure may contain at least one skeletal heteroatom selected from N, S, and O, and are optionally further substituted with 1-4 C1-3 alkyl or C1-3 perfluoroalkyl; Y is selected from CHR1, O, S, and NR1;
E is selected from CH2 and O;
G is selected from CH, and N;
further wherein when G is CH and X is phenyl, Z is not phenyl;
the linkage of G to X may optionally be selected from C1-3 alkyl and ethylene oxide, each of which may be substituted with 1-4 heteroatoms independently selected from N, S, and O and are optionally further substituted with 1-4 C1-3 alkyl;
the linkage of X to Z may occupy any available position on X and Z;
substituent NR1R2 may occupy any available position on Z;
R1 is selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH2, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylaminoC(O)—;
when R1 is H, R2 may be selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH2, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylaminoC(O)—;
when R1 is H, linear or branched alkyl of 1-8 carbons, or heteroaryl, R2 may be a functional group selected from

[(C═O)CH(W)NH]m-[C═O]—[V]k-J,

(C═O)OCH2-p-aminophenyl-N—V-J,

(C═O)OCH2-p-aminophenyl-N—[(C═O)CH(W)NH]m-[C═O]—[V]k-J, and

[V]k-(C═O)OCH2-p-aminophenyl-N—[(C═O)CH(W)NH]m-[C═O]-J,
wherein m=1-6, k=0-1, and each permutation of W may independently be selected from H, [(CH2)nR3] where n=1-4, a branched alkyl chain terminating in R3, and a linear or branched polyethylene oxide group comprising 1-13 units;
R3 is selected from H, methyl, ethyl, isopropyl, OH, O-alkyl, NH2, NH-alkyl, N-dialkyl, SH, S-alkyl, guanidine, urea, carboxylic acid, carboxamide, carboxylic ester, substituted or unsubstituted aryl, substituted or unsubstituted heteroaryl, wherein said aryl and heteroaryl substituents may be selected from alkyl, haloalkyl, halogen, biphenyl, nitro, nitrile, —OH, —O-alkyl, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, and dialkylaminoC(O)—;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH2, N3, thio, cyclooctyne, —OH, —CO2H, trans-cyclooctene, alkynyl, propargyl,

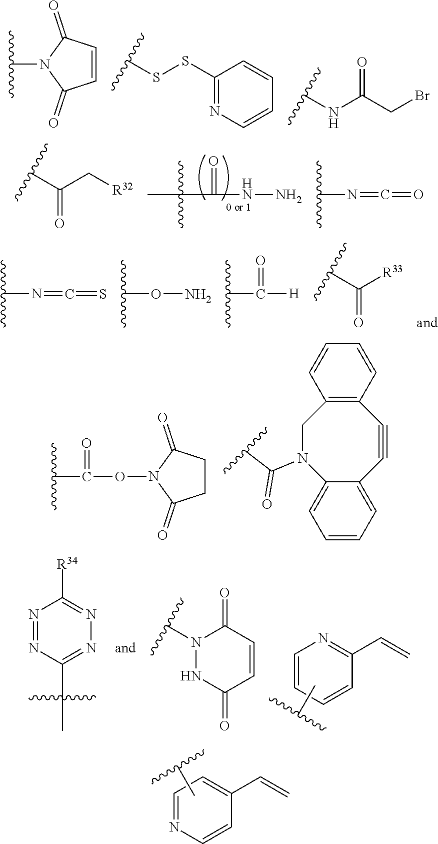

where R32 is selected from Cl, Br, F, mesylate, and tosylate and R33 is selected from Cl, Br, I, F, OH, —O—N-succinimidyl, —O-(4-nitrophenyl), —O-pentafluorophenyl or —O-tetrafluorophenyl R34 is H, Me, tetrazine-H, and tetrazine-Me;
R5 is selected from the group consisting of —CH₂OH, —CH2SH, —CH2Cl, —SCH2Cl, —SCH2F, —SCH2CF3, hydroxy, —OCH2CN, —OCH2Cl, —OCH2F, —OCH3, —OCH2CH3, —SCH2CN, and

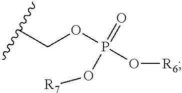

R6 and R7 are independently selected from hydrogen and C1-10 alkyl;
Q may be H,

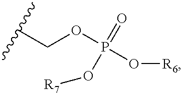

C(O)R8 where R8 is linear or branched alkyl of 1-8 carbons, or (C═O)NR4CHnNR4(C═O)OCH2-(V)n-J where n=1-4 and R4=H, alkyl or branched alkyl, or P(O)OR4;
A1 and A2 are independently selected from H and F; and
unless otherwise specified, all possible stereoisomers are claimed.

Further provided are glucocorticoid agonist compounds which possess the structure of Formula (II):

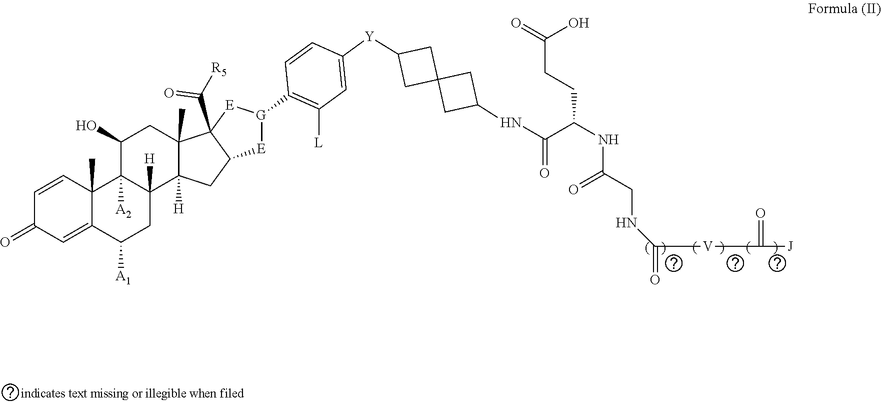

wherein
Y is selected from CH2 and O;
E is selected from CH2 and O;
G is selected from CH, and N;
L is selected from H and F;
R5 is selected from —CH₂OH, —SCH2F and

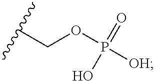 [SMILES: *COP(=O)(O)O]

A1 and A2 are independently selected from H and F;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH2, N3, thio, cyclooctyne, —OH, —CO2H, trans-cyclooctene, alkynyl, propargyl,

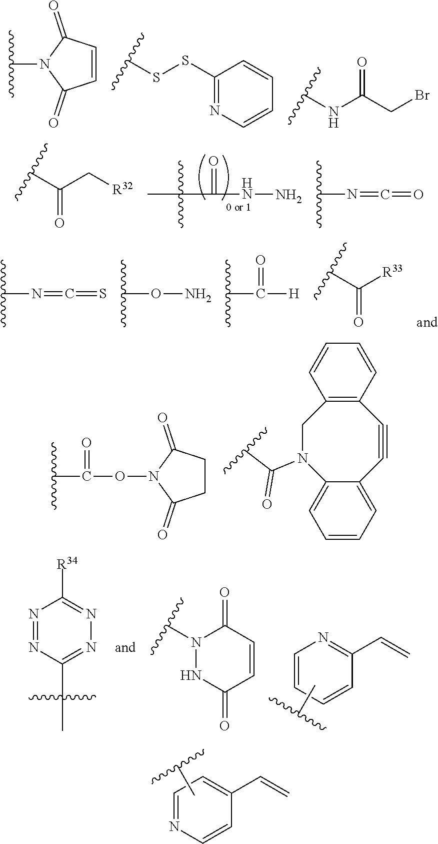

where R₃₂ is selected from Cl, Br, F, mesylate, and tosylate and R33 is selected from Cl, Br, I, F, OH, —O—N-succinimidyl, —O-(4-nitrophenyl), —O-pentafluorophenyl or —O-tetrafluorophenyl R34 is H, Me, tetrazine-H, and tetrazine-Me.

Also provided are glucocorticoid agonist compounds which possess the structure of Formula (III):

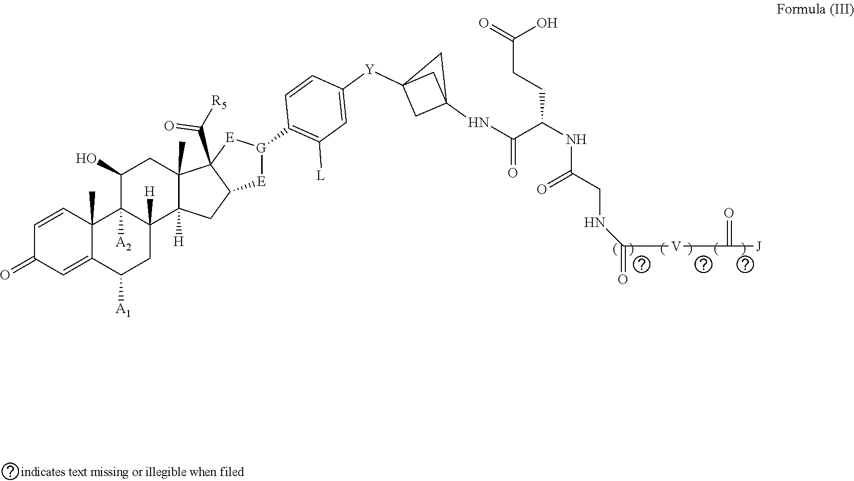

wherein
Y is selected from CH2 and O;
E is selected from CH2 and O;
G is selected from CH, and N;
L is selected from H and F;
R5 is selected from —CH₂OH, —SCH2F and

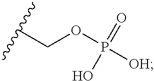 [SMILES: *COP(=O)(O)O]

A1 and A2 are independently selected from H and F;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH2, N3, thio, cyclooctyne, —OH, —CO2H, trans-cyclooctene, alkynyl, propargyl,

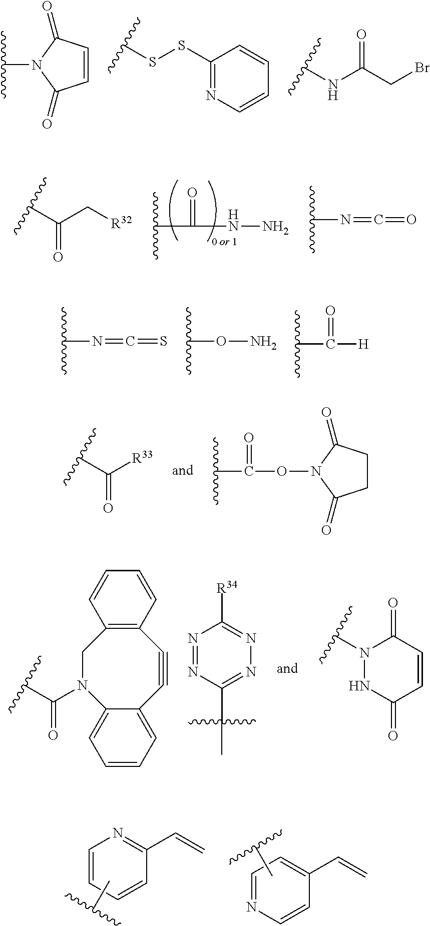

where R32 is selected from Cl, Br, F, mesylate, and tosylate and R33 is selected from Cl, Br, I, F, OH, —O—N-succinimidyl, —O-(4-nitrophenyl), —O-pentafluorophenyl or —O-tetrafluorophenyl R34 is H, Me, tetrazine-H, and tetrazine-Me.

Also provided are glucocorticoid agonist-linker compounds which comprise a glucocorticoid agonist having the structure of Formula (I), (II) or (III), attached to a cleavable or non-cleavable linker.

Further provided are ADCs comprising an antibody that binds to an immune cell antigen, e.g., VISTA, attached to a glucocorticoid agonist having the structure of Formula (I), (II) or (III), which is in turn attached to a cleavable or non-cleavable linker.

Additionally provided are compositions and medicaments comprising said glucocorticoid agonists, glucocorticoid agonist-linkers and ADCs containing same.

Further provided are therapeutic and prophylactic uses of such glucocorticoid agonists, glucocorticoid agonist-linkers and ADCs containing same, especially for treating inflammatory, allergic and autoimmune conditions.

With that general understanding unless defined otherwise, all technical and scientific terms used herein have the same meaning as those commonly understood by one of ordinary skill in the art to which this invention belongs. Although methods and materials similar or equivalent to those described herein may be used in the invention or testing of the present invention, suitable methods and materials are described herein. The materials, methods and examples are illustrative only, and are not intended to be limiting. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation, and delivery, and treatment of patients.

### Definitions

To facilitate an understanding of the present disclosure, a number of terms and phrases are defined below.

As used in the description herein and throughout the claims that follow, the meaning of “a,” “an,” and “the” includes plural reference unless the context clearly dictates otherwise.

In the present disclosure, the term “glucocorticosteroid” or “steroid” refers to naturally-occurring or synthetic steroid hormones that interact with glucocorticoid receptors. Non-limiting exemplary glucocorticosteroids include those described in WO 2009/069032, US20180126000, WO05/028495 among others and preferably refer to the novel glucocorticoid agonists of Formula I, II or III, and glucocorticoid agonist-linkers and ADCs containing same disclosed herein. Non-limiting examples of known glucocorticosteroids include:

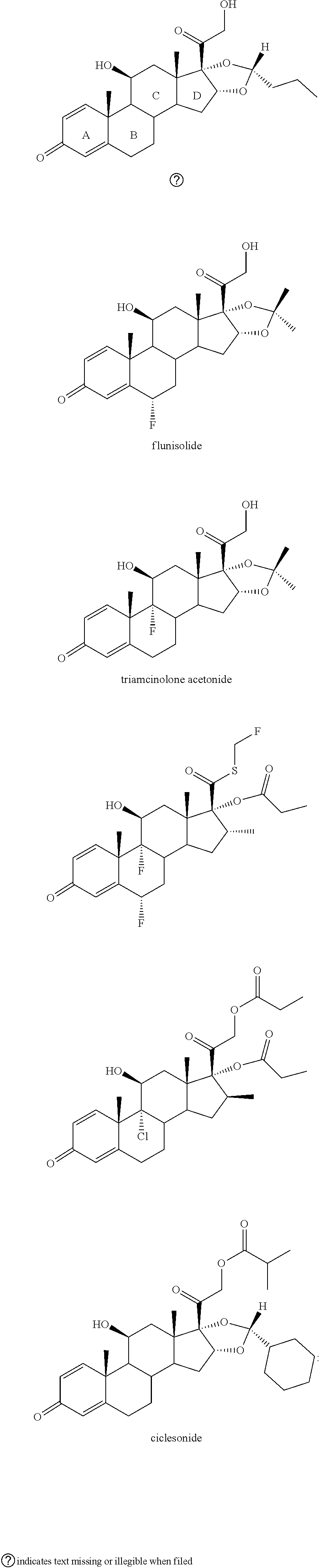

Other known glucocorticosteroids are described in WO 2009/069032. Specific examples of glucocorticosteroids include, 16-alpha hydroxyprednisolone, dexamethasone, difluorasone, flumethasone, flunisolide, fluocinolone acetonide, fluticasone propionate, ciclesonide, methylprednisolone, prednisone, prednisolone, mometasone, triamcinolone acetonide and the novel glucocorticoid agonists of Formula I, II or III, and glucocorticoid agonist-linkers and ADCs containing same disclosed herein.

A “glucocorticosteroid derivative” is a compound derived by the addition or removal of one or more atoms or functional groups in order to facilitate attachment of the “glucocorticosteroid derivative” to another moiety, e.g., a linker and/or an antibody or antibody fragment. Generally, this addition or removal will not preclude the activity of the “glucocorticosteroid derivative”, i.e., its ability to elicit anti-inflammatory activity upon internalization by an immune cell. “Glucocorticosteroid derivatives” specifically include a “radical of a glucocorticosteroid” or a “glucocorticosteroid radical”.

A “radical of a glucocorticosteroid” or a “glucocorticosteroid radical” is produced by the removal of one or more atoms from a parent glucocorticosteroid, i.e., hydrogen atoms, in order to facilitate the attachment of the parent glucocorticosteroid to another moiety, typically a linker. For example, a hydrogen atom may be removed from any suitable —NH₂ group of the parent glucocorticosteroid; a hydrogen atom may be removed from any suitable —OH group of the parent glucocorticosteroid a hydrogen atom may be removed from any suitable —SH group; a hydrogen atom maty be removed from any suitable —N(H)— group; a hydrogen atom is removed from any suitable —CH₃, —CH₂— or —CH═ group of the parent glucocorticosteroid.

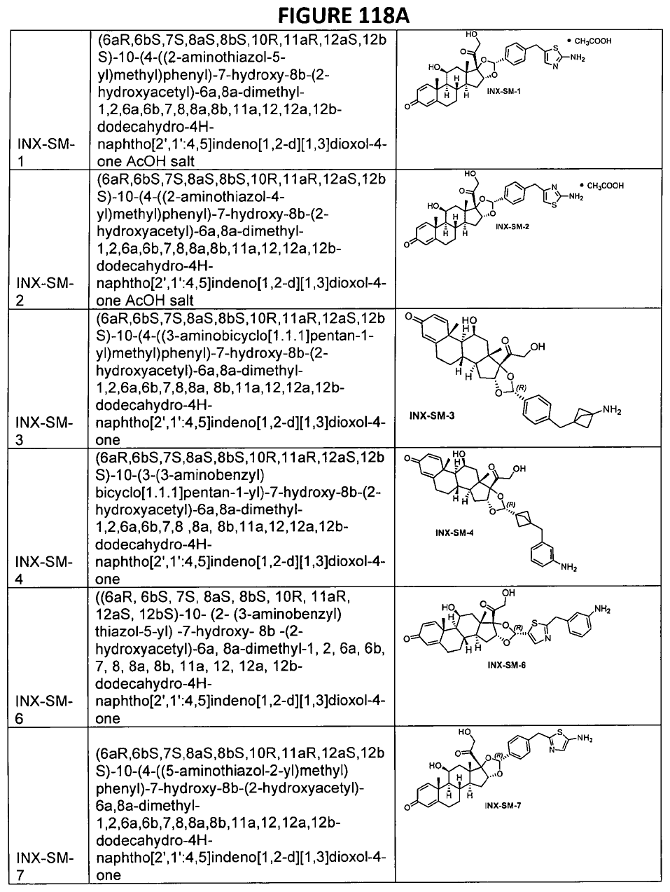

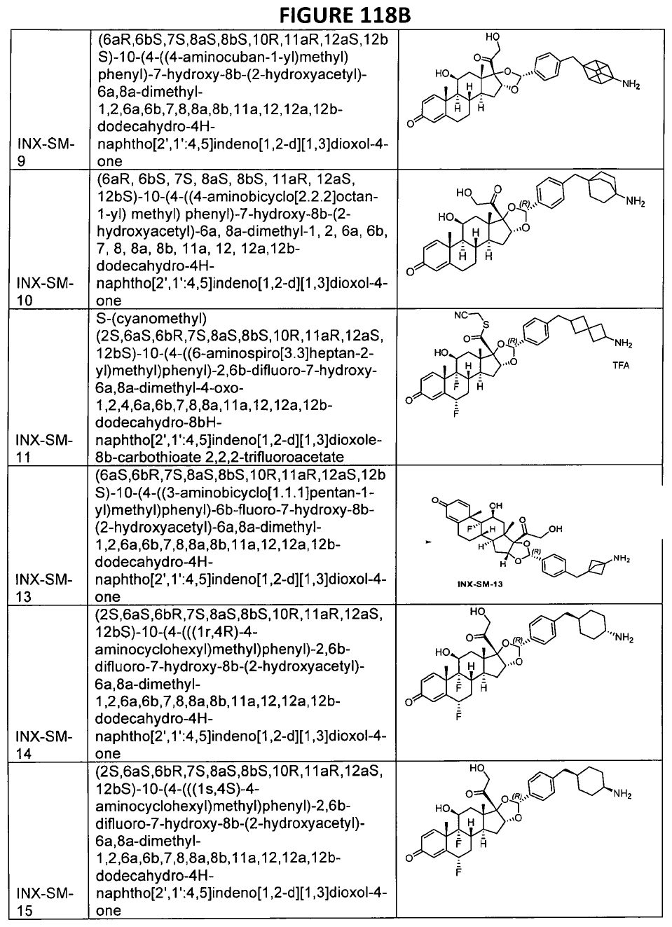

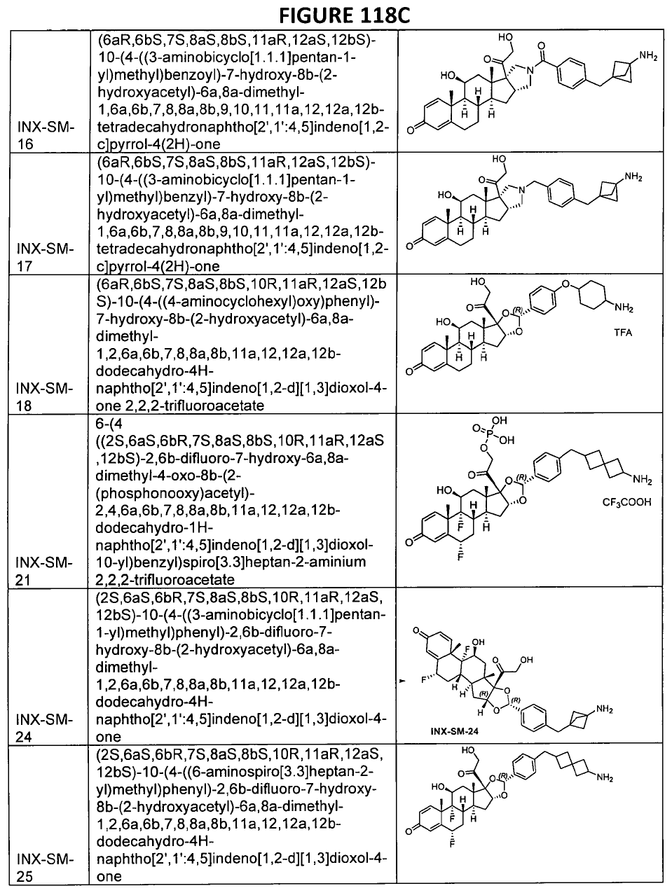

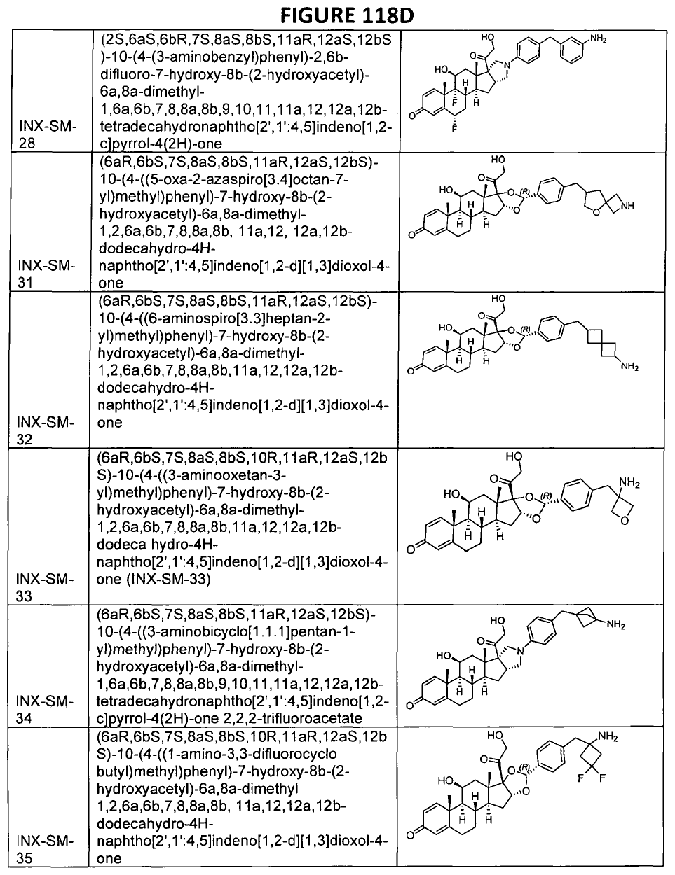

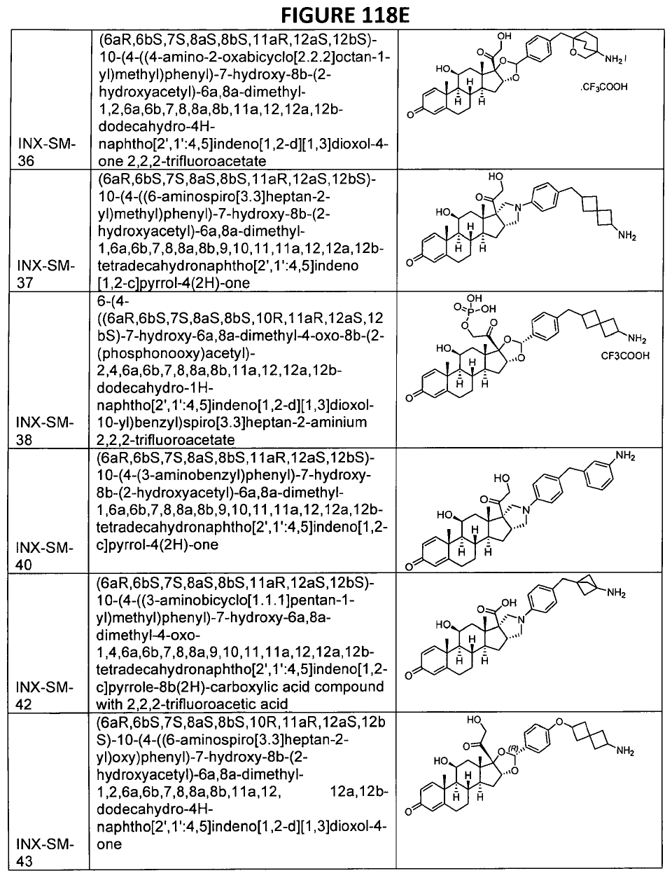

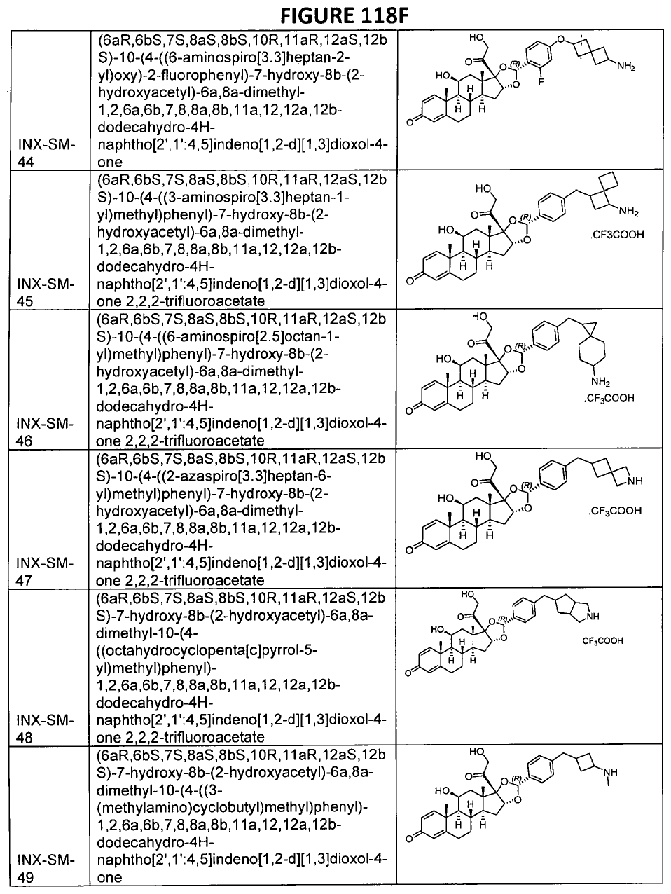

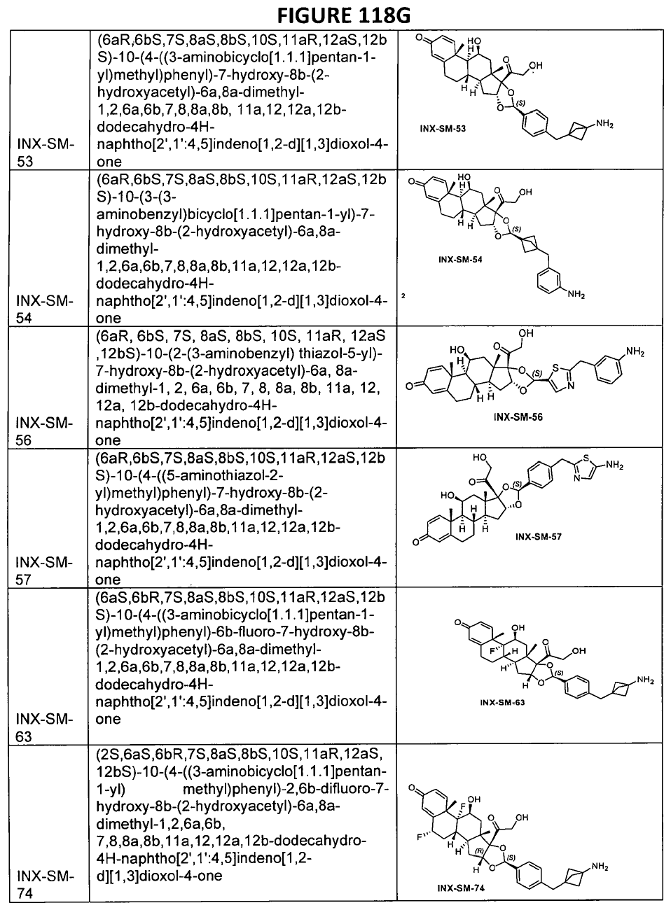

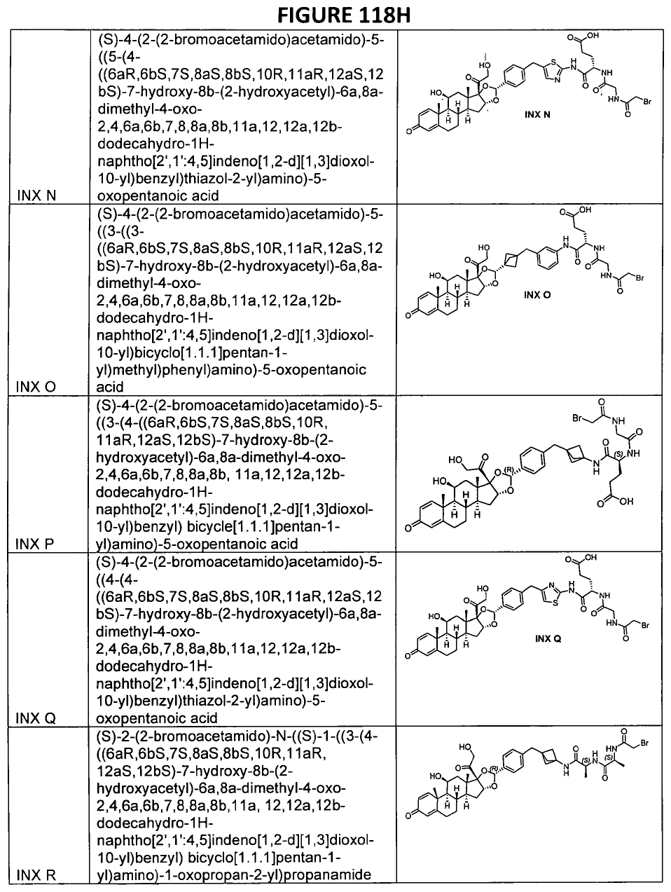

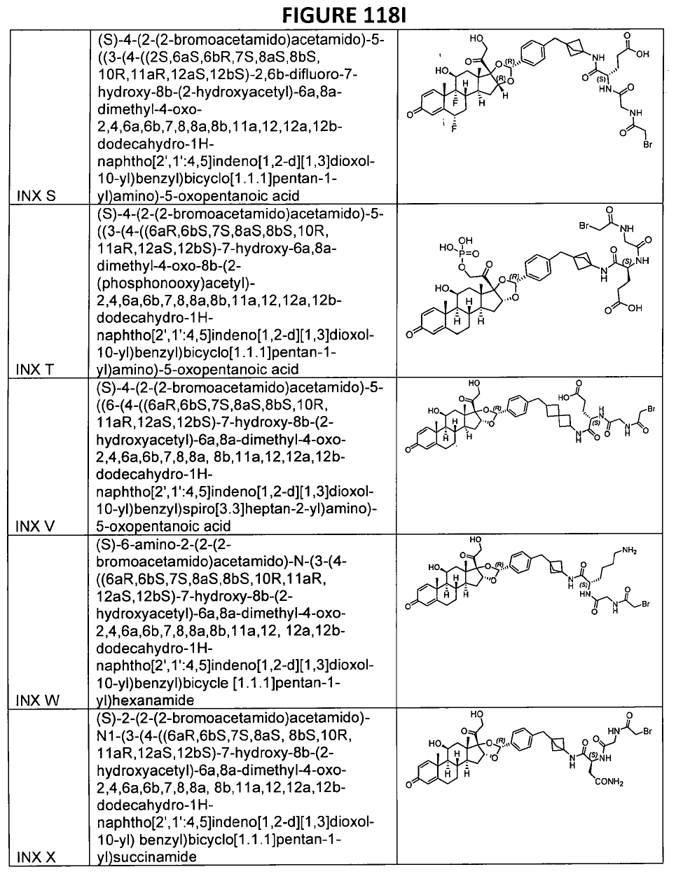

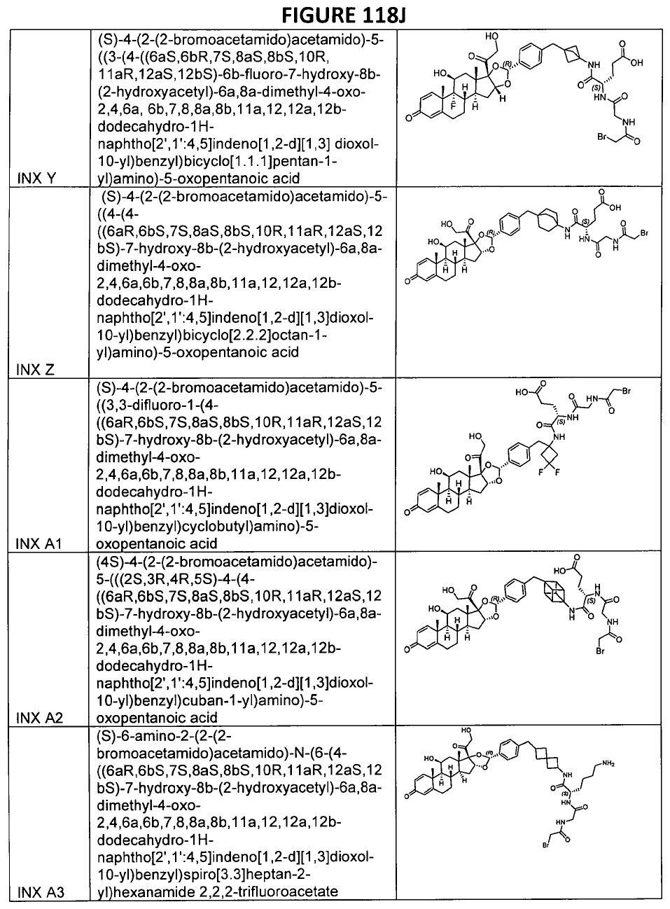

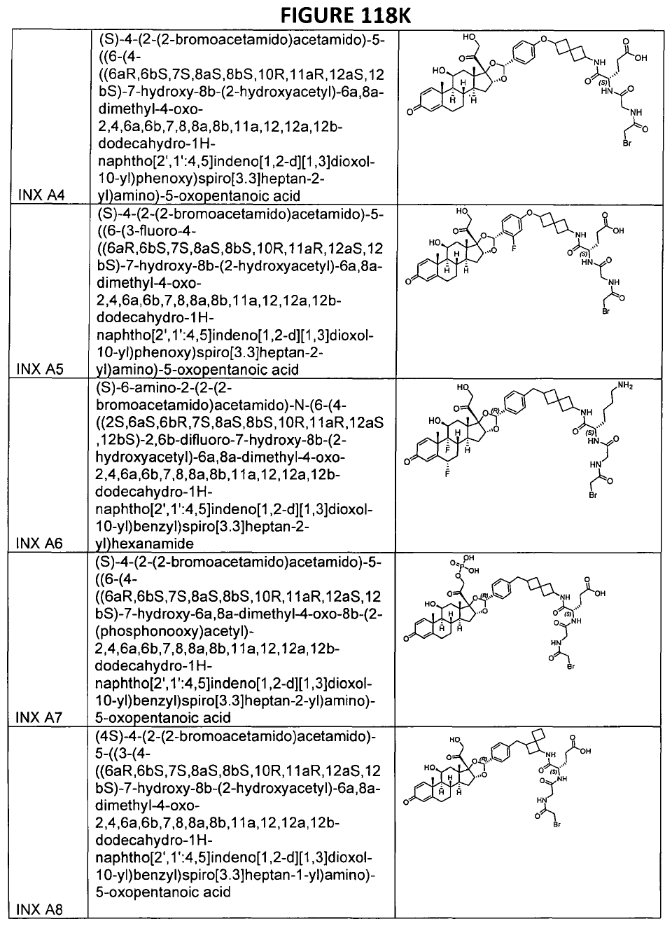

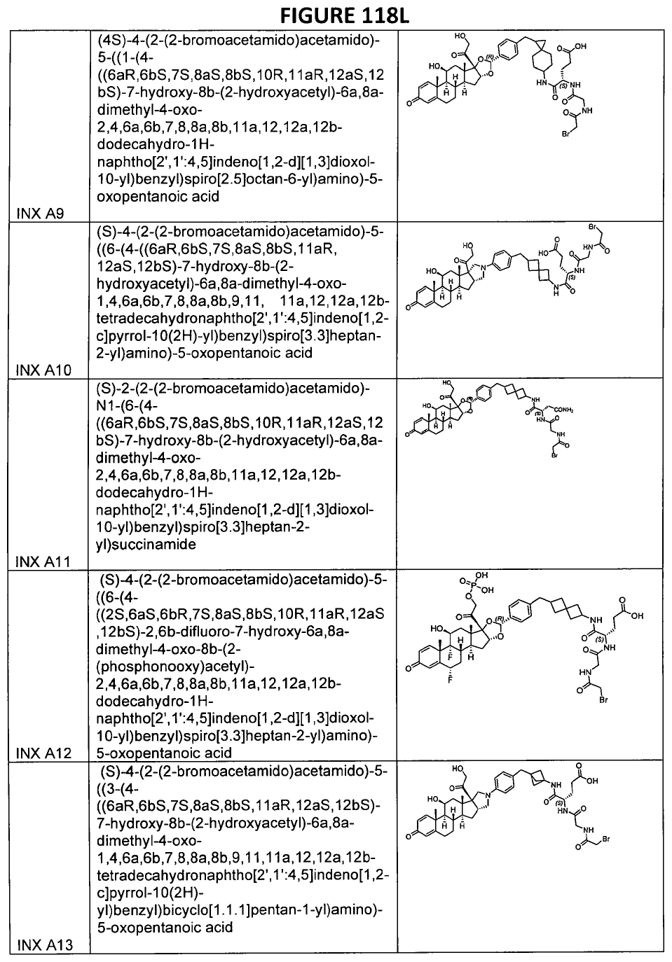

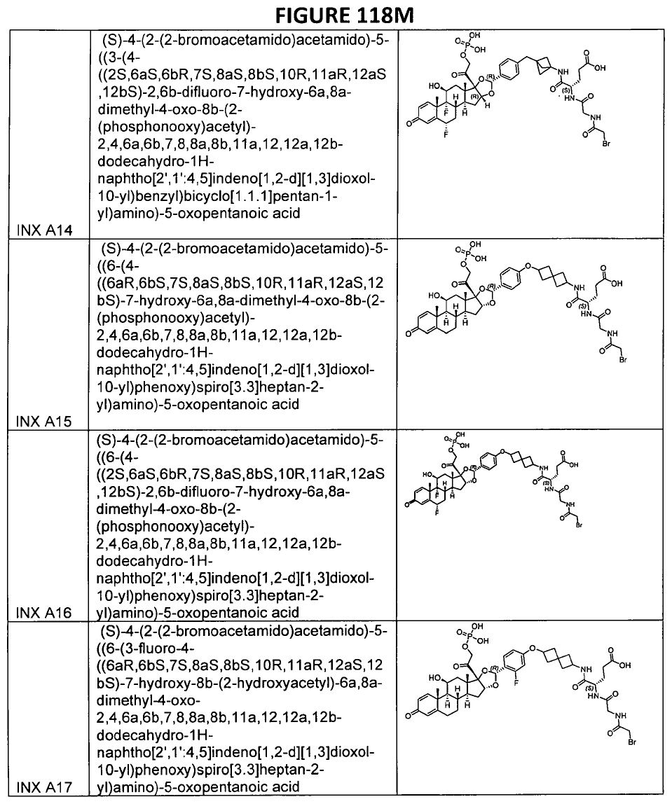

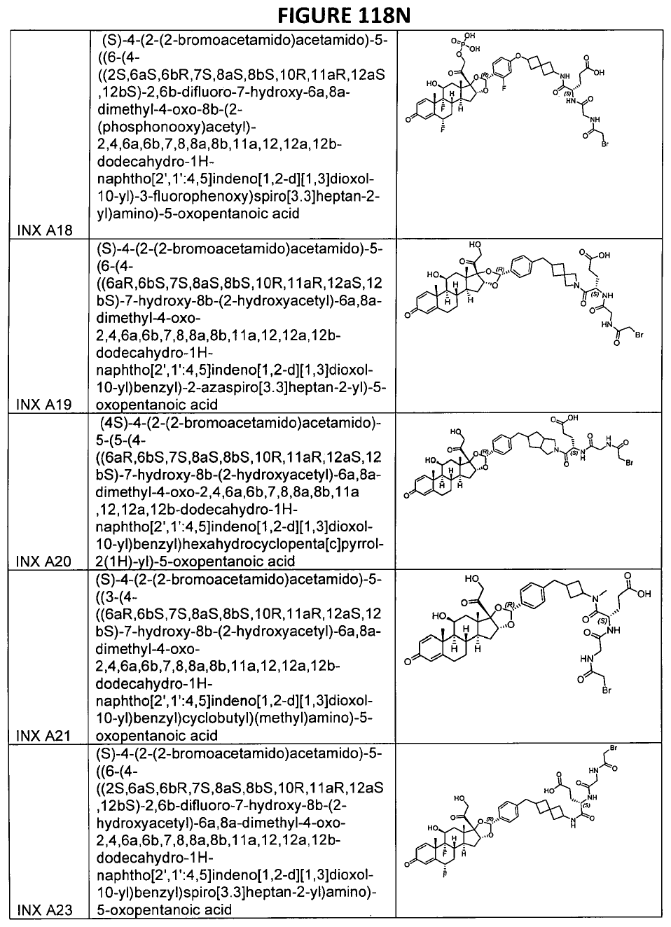

In the present disclosure, the term “heterobifunctional group” or the term “heterotrifunctional group” refers to a chemical moiety ((“Q”) in the generic formula for ADCs disclosed herein) that optionally may be used to connect a linker and the anti-VISTA antibody or antibody fragment. Heterobi- and tri-functional groups are characterized as having different reactive groups at either end of the chemical moiety. Non-limiting exemplary heterobifunctional groups are disclosed in US Publication No.: 20180126000, incorporated by reference herein and which are further exemplified in the ADC conjugates disclosed in the Exemplary Embodiments section and in the examples, e.g., Example 3 and the compounds in FIG. 118A-O of this application.

Heterobi- and tri-functional groups are well known in the art for producing protein conjugates and antibody drug conjugates (ADCs) specifically. These moieties are characterized as having different reactive groups at either end of the chemical moiety. Non-limiting exemplary heterobifunctional groups include:

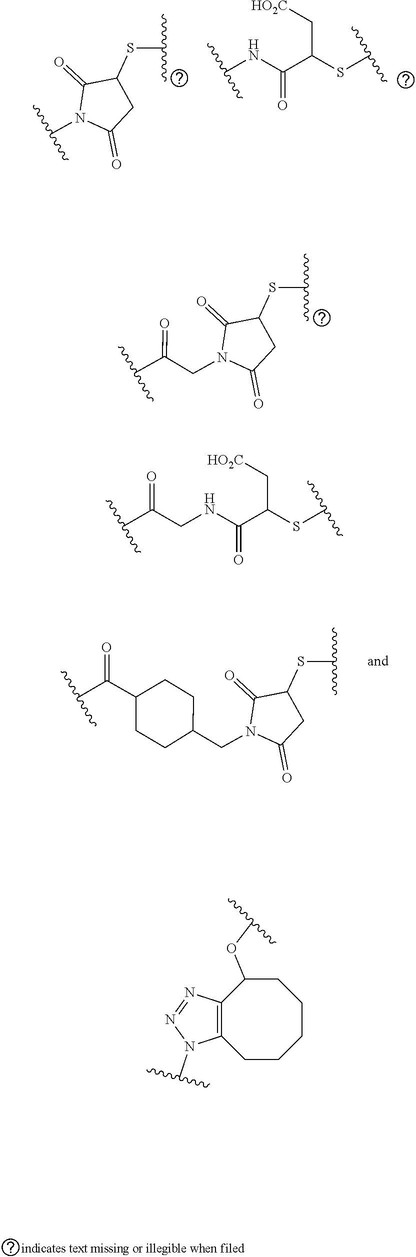 [SMILES: *SC1CC(=O)N(*)C1=O ; *NC(=O)C(CC(=O)O)S* ; *SC1CC(=O)N(CC(*)=O)C1=O ; *SC(CC(=O)O)C(=O)NCC(*)=O ; *SC1CC(=O)N(CC2CCC(C(*)=O)CC2)C1=O ; *OC1CCCCCc2c1nnn2*]

An exemplary heterotrifunctional
I group is:

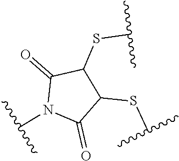 [SMILES: *SC1C(=O)N(*)C(=O)C1S*]

As used herein, the terms “antibody” and “antibodies” are terms of art and can be used interchangeably herein and refer to a molecule with an antigen-binding site that specifically binds an antigen.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc. As used herein, the term “antibody” encompasses bispecific and multispecific antibodies.

The term “antibody fragment” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)₂, and Fv fragments, linear antibodies, and single chain antibodies. An “antigen-binding fragment” can be a bispecific or multispecific antigen-binding fragment.

A “blocking” antibody or an “antagonist” antibody is one which inhibits or reduces biological activity of the antigen it binds, such as VISTA. In some embodiments, blocking antibodies or antagonist antibodies substantially or completely inhibit the biological activity of the antigen. The biological activity can be reduced by 10%, 20%, 30%, 50%, 70%, 80%, 90%, 95%, or even 100%.

A “promoting” antibody or an “enhancing” antibody an “agonist” antibody is one which enhances or increases a biological activity of the antigen it binds, such as VISTA. In some embodiments, blocking antibodies or antagonist antibodies substantially or completely inhibit the biological activity of the antigen. The biological activity can be reduced by 10%, 20%, 30%, 50%, 70%, 80%, 90%, 95%, or even 100%.

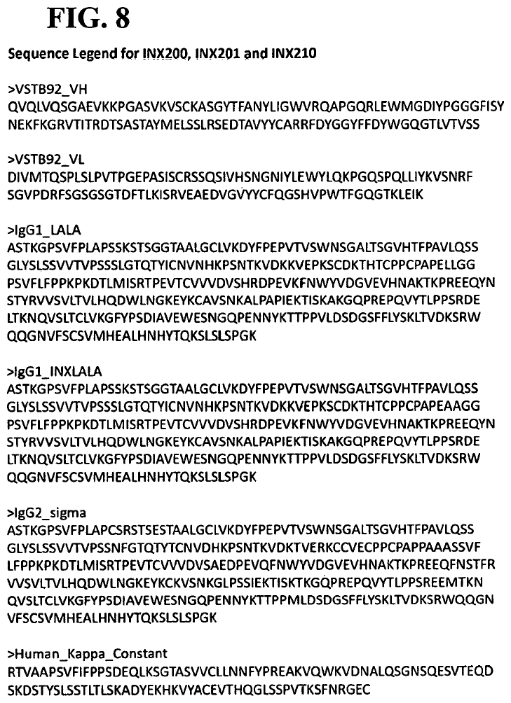

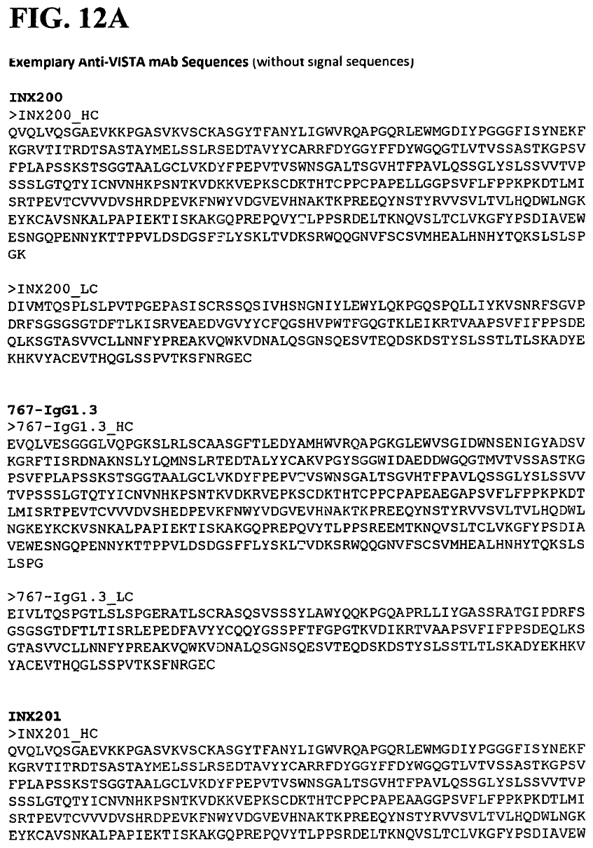

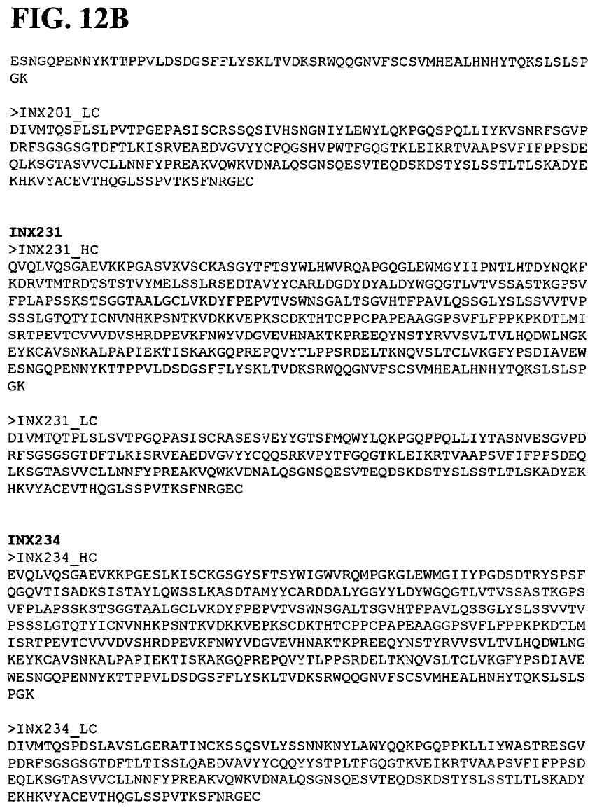

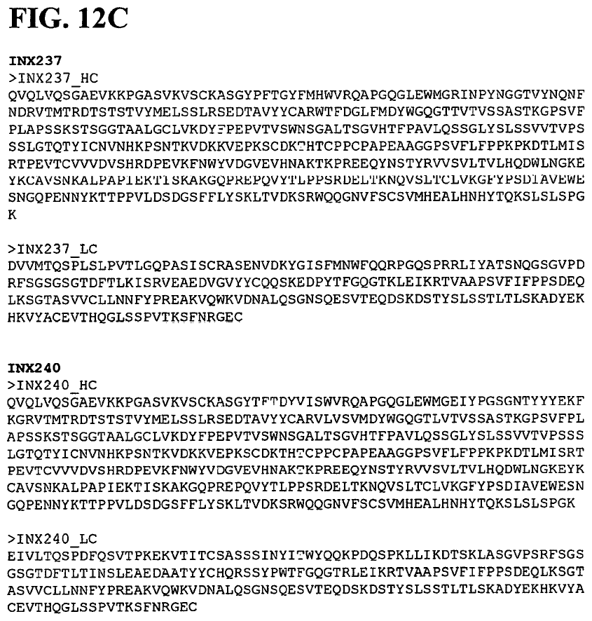

The term “anti-VISTA antibody” or “an antibody that binds to VISTA” refers to an antibody that specifically binds VISTA, generally human VISTA with sufficient affinity such that the antibody is useful for targeting VISTA expressing immune cells. The extent of binding of an anti-VISTA antibody to an unrelated, non-VISTA protein can be less than about 10% of the binding of the antibody to VISTA as measured, e.g., by a radioimmunoassay (RIA). In certain embodiments, an antibody that binds to VISTA has a dissociation constant (Kd) of ≤1 μM, ≤100 nM, ≤10 nM, ≤1 nM, or ≤0.1 nM. Exemplary anti-VISTA antibodies and fragments comprised in the subject ADCs will comprise the same CDRs and/or same variable heavy and light chin polypeptides as in an of VSTB94 or VSTB49-116, i.e., respectively having the sequences shown in FIG. 8, 10 and FIG. 12.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)₂, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91 (3): 969-973 (1994), and Roguska et al., Protein Eng. 9 (10): 895-904 (1996). In some embodiments, a “humanized antibody” is a resurfaced antibody.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). Unless explicitly indicated otherwise, the numbering system used herein is the Kabat numbering system.

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

In certain aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In certain aspects, the CDRs of an antibody or antigen-binding fragment thereof can be determined according to the IMGT numbering system as described in Lefranc M-P, (1999) The Immunologist 7:132-136 and Lefranc M-P et al., (1999) Nucleic Acids Res 27:209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

In certain aspects, the CDRs of an antibody or antigen-binding fragment thereof can be determined according to MacCallum R M et al., (1996) J Mol Biol 262:732-745. See also, e.g., Martin A. “Protein Sequence and Structure Analysis of Antibody Variable Domains,” in Antibody Engineering, Kontermann and Dubel, eds., Chapter 31, pp. 422-439, Springer-Verlag, Berlin (2001).

In certain aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.).

A “constant region” of an antibody refers to the constant region of the antibody light chain or the constant region of the antibody heavy chain, either alone or in combination.

The term “human” antibody means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric” antibodies refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc.) with the desired specificity, affinity, and capability while the constant regions are homologous to the sequences in antibodies derived from another (usually human) to avoid eliciting an immune response in that species.

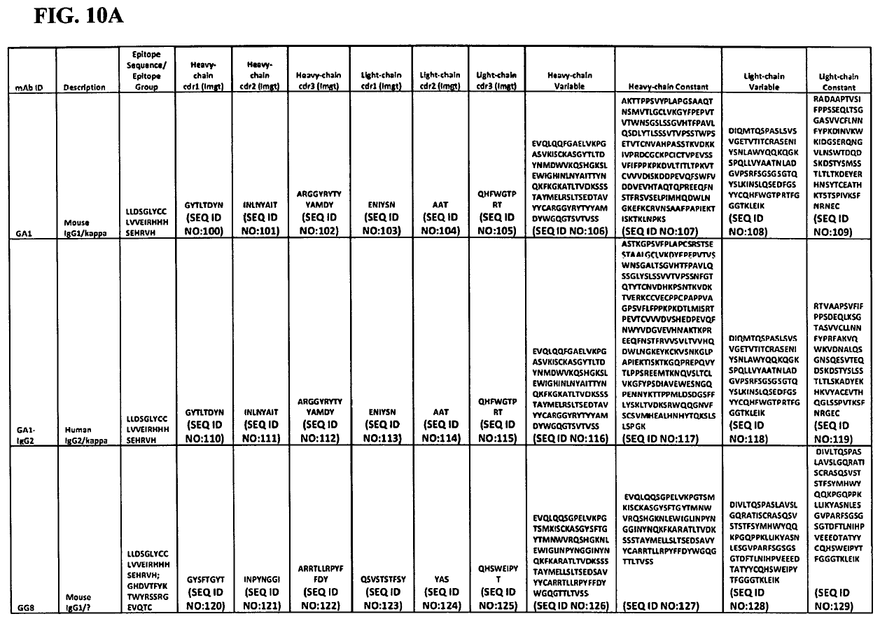

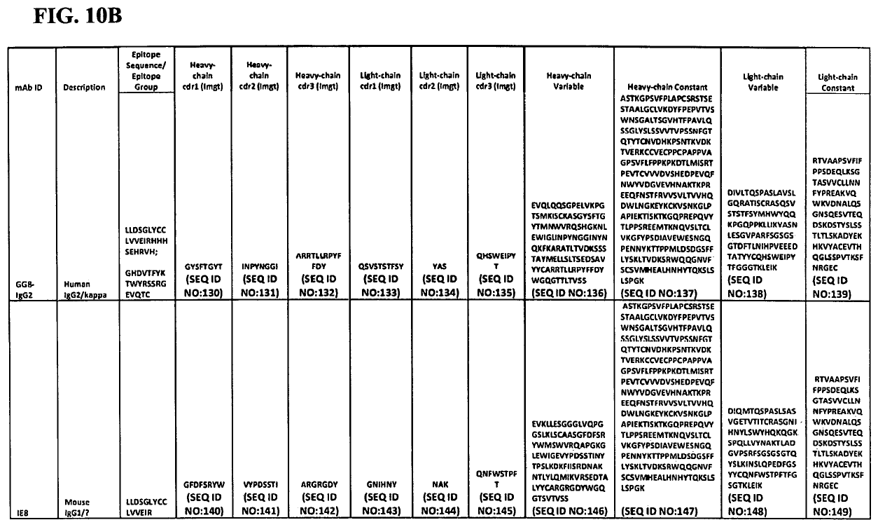

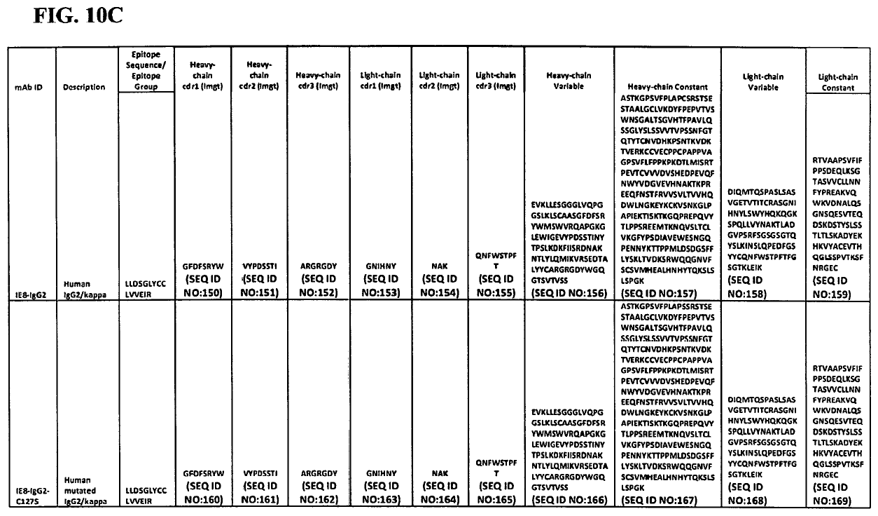

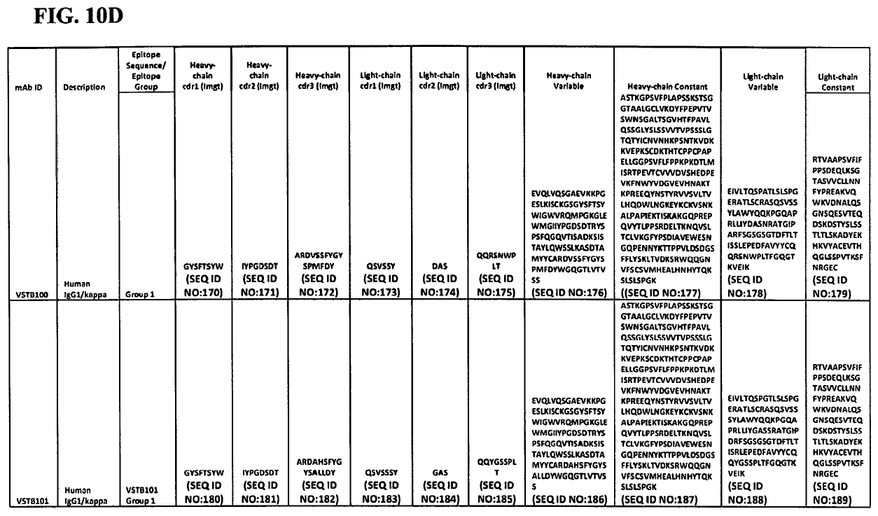

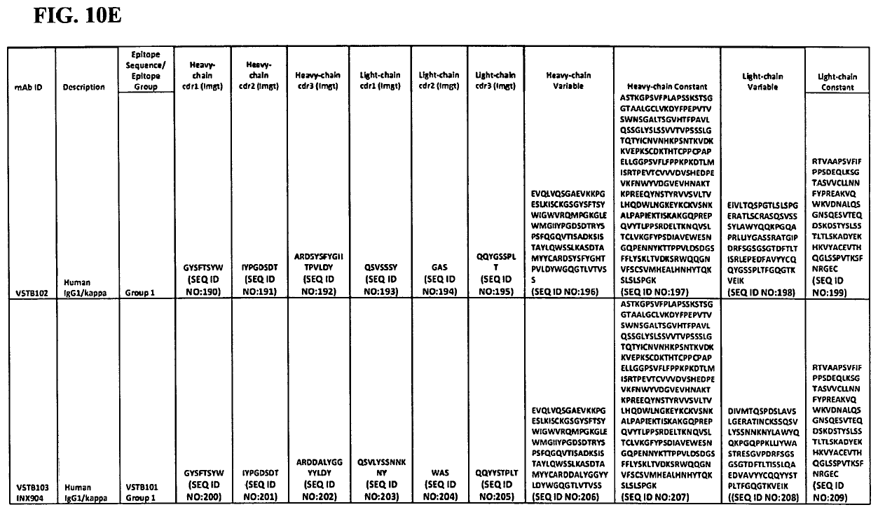

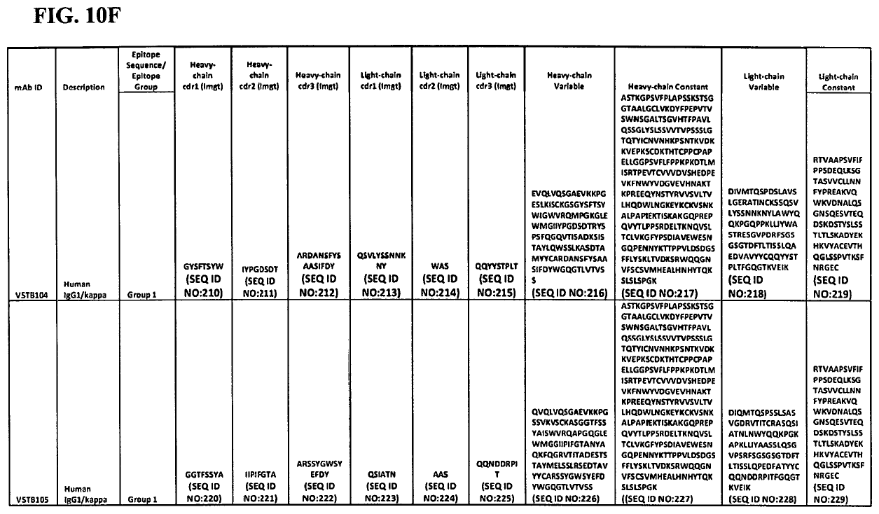

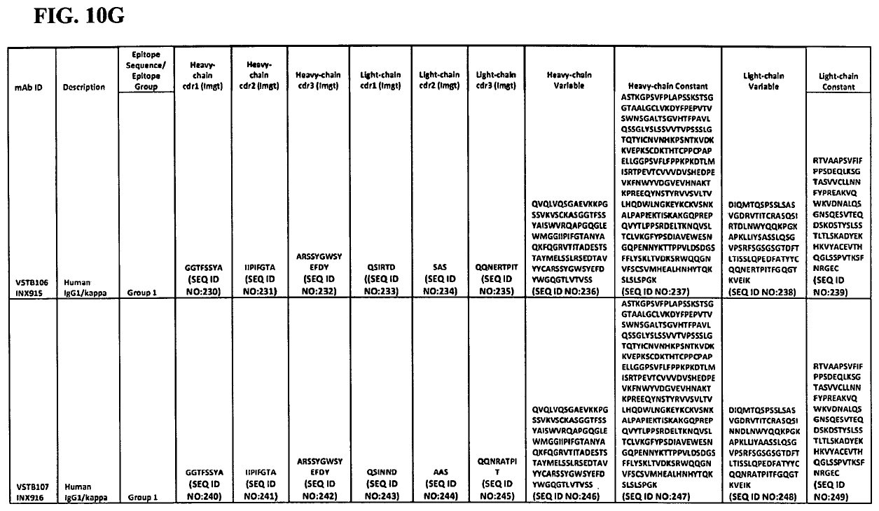

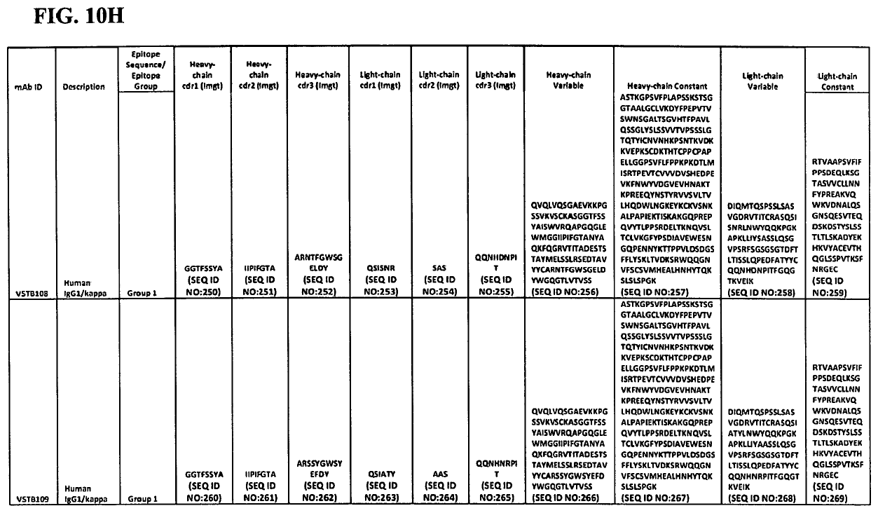

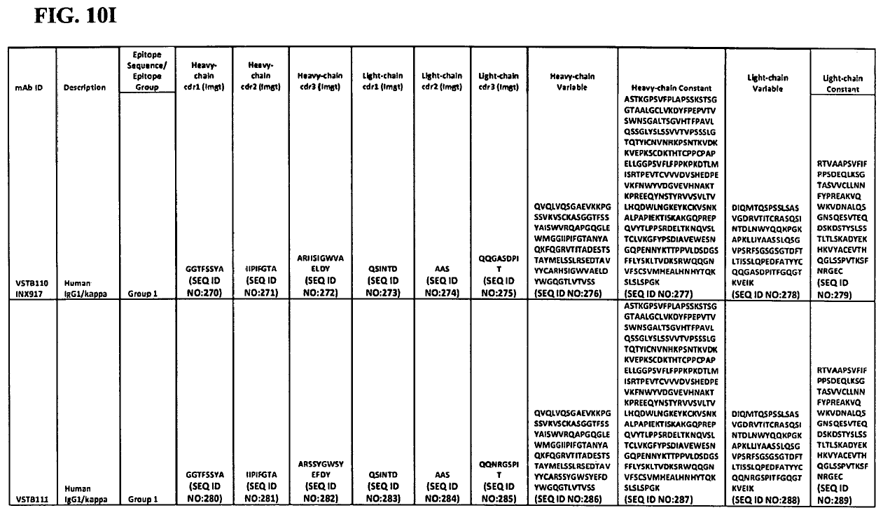

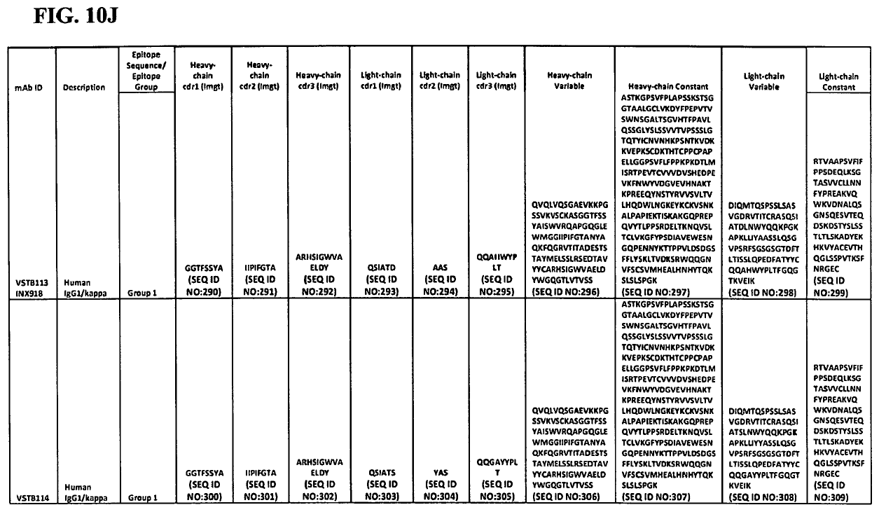

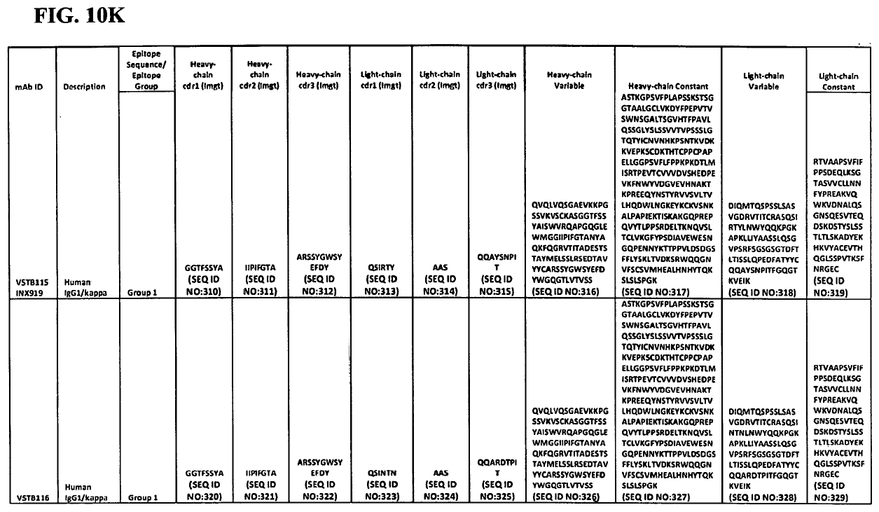

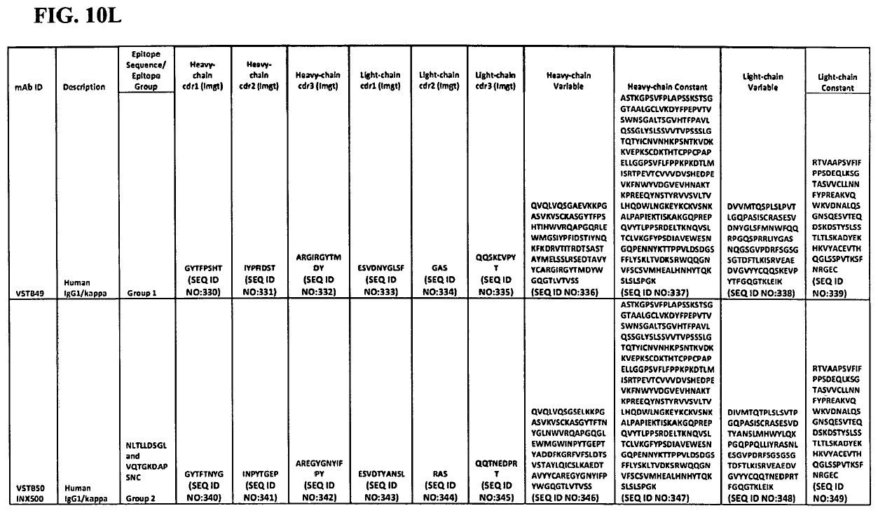

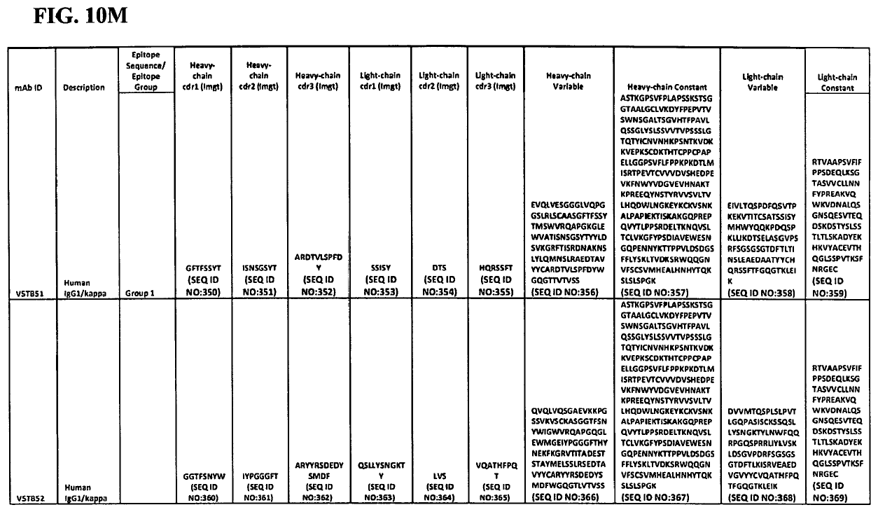

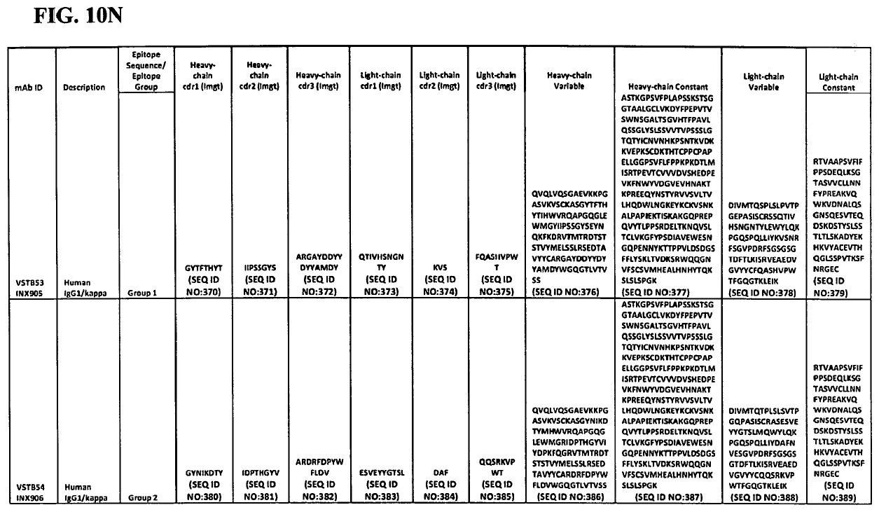

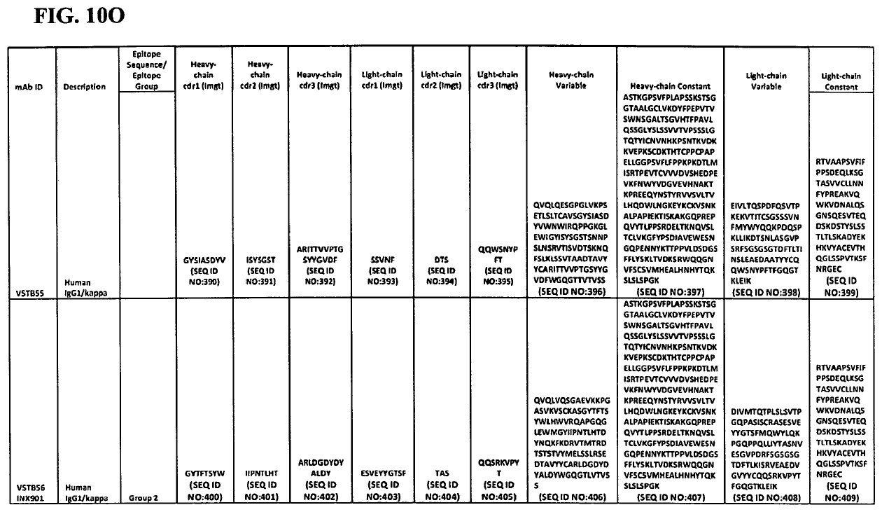

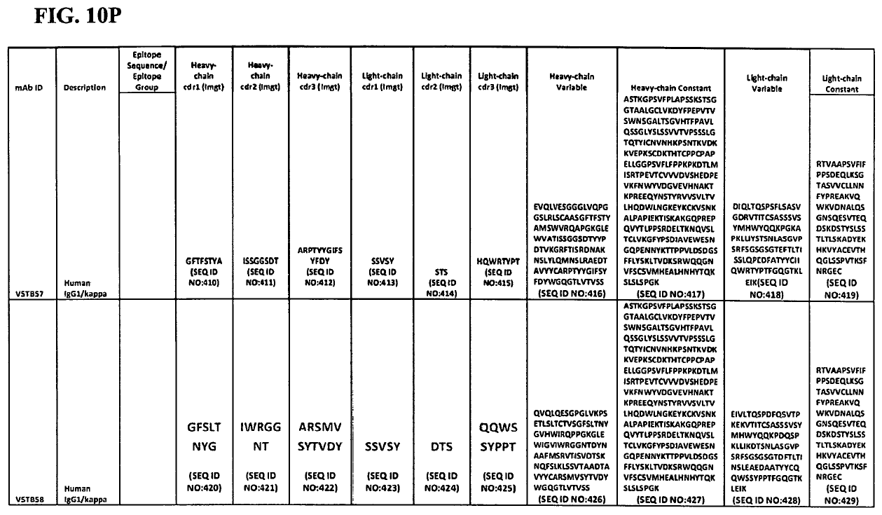

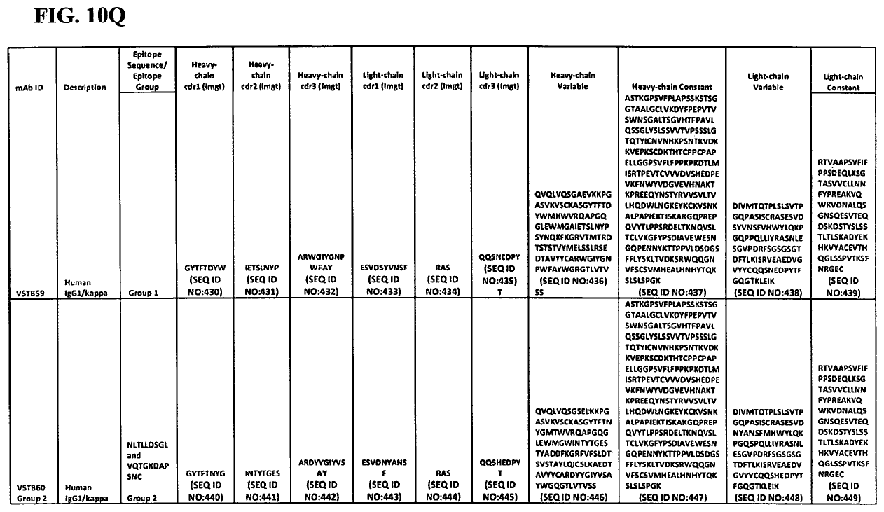

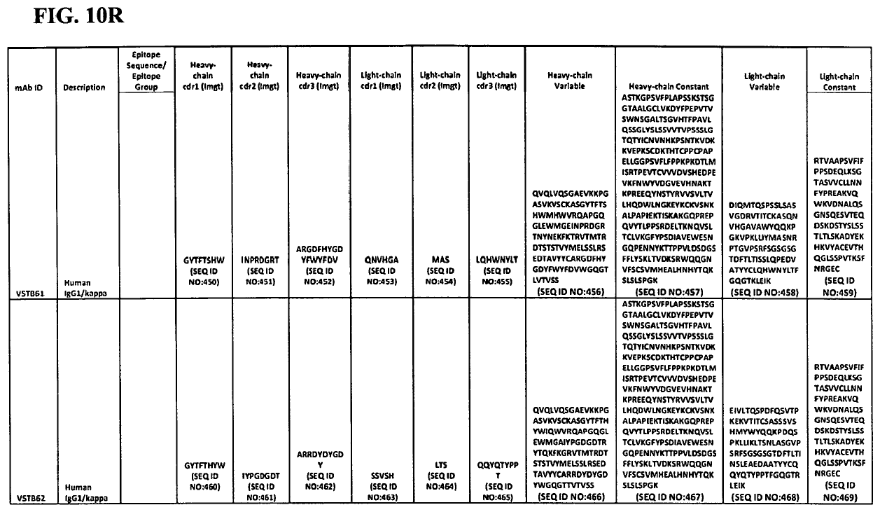

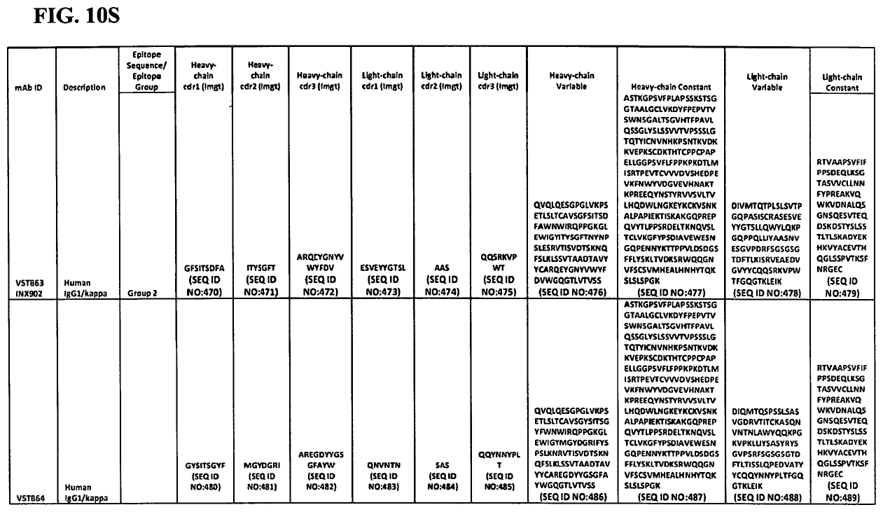

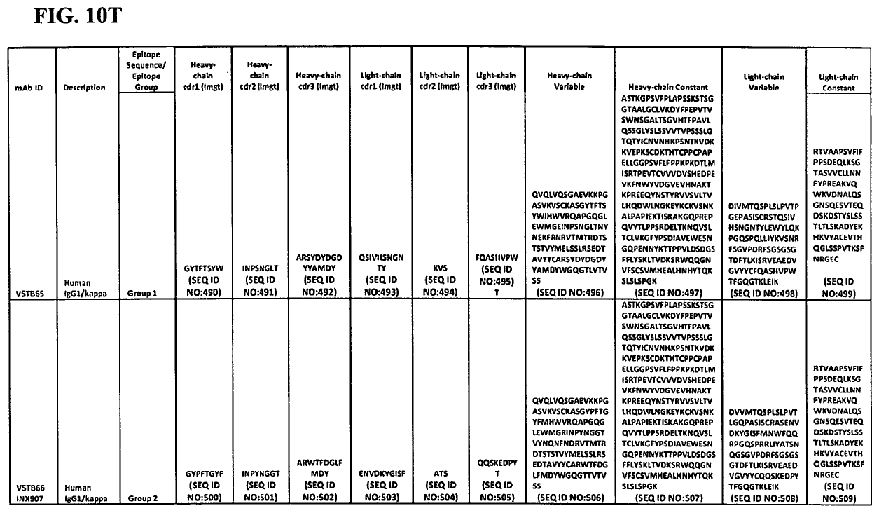

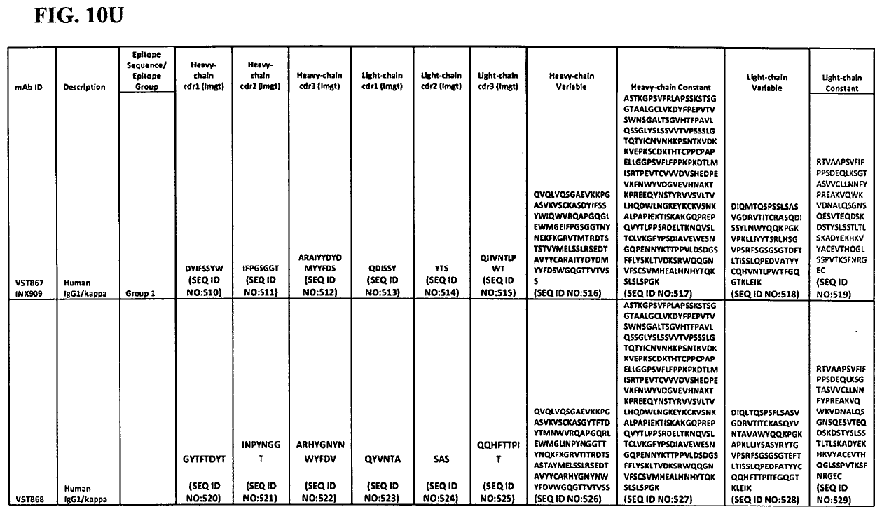

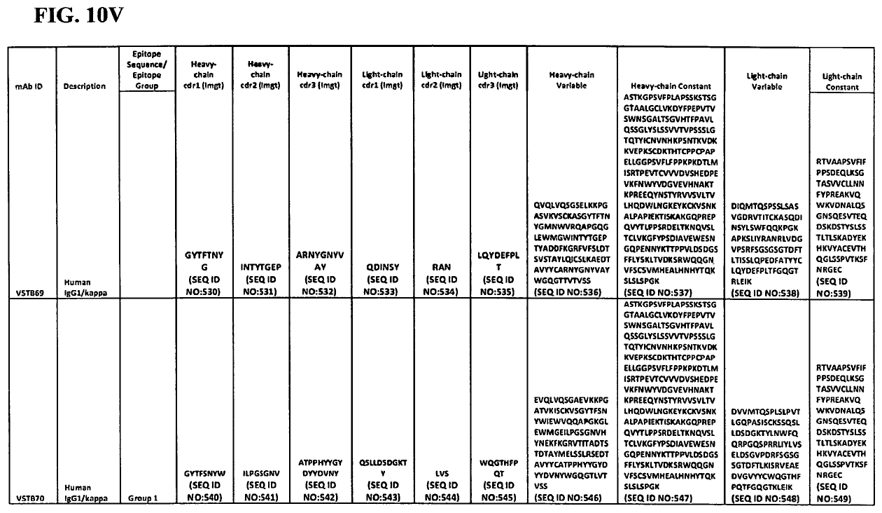

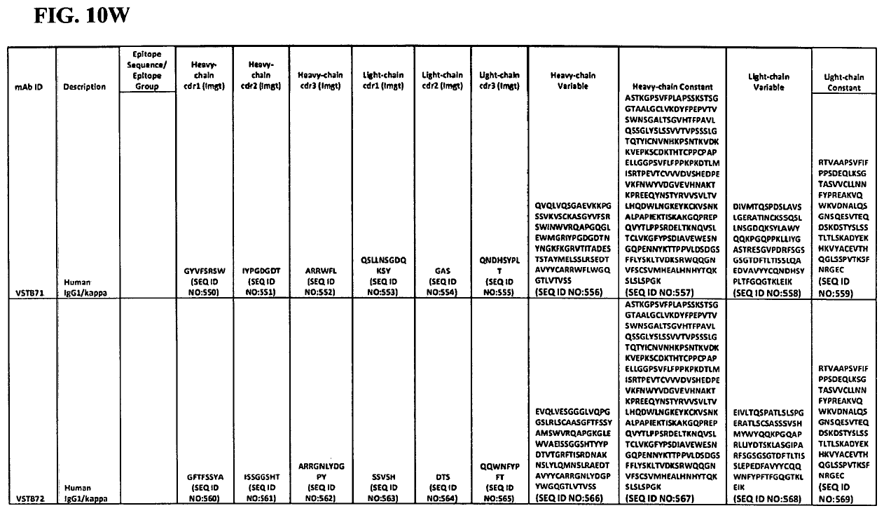

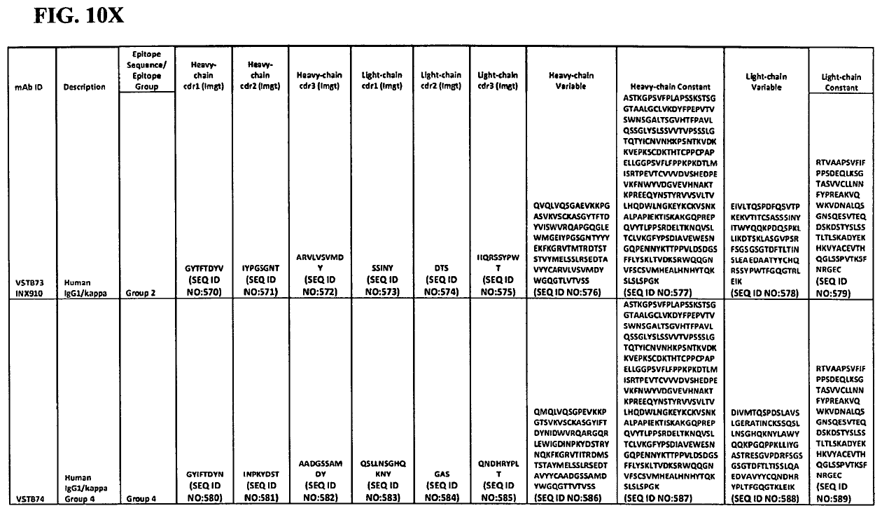

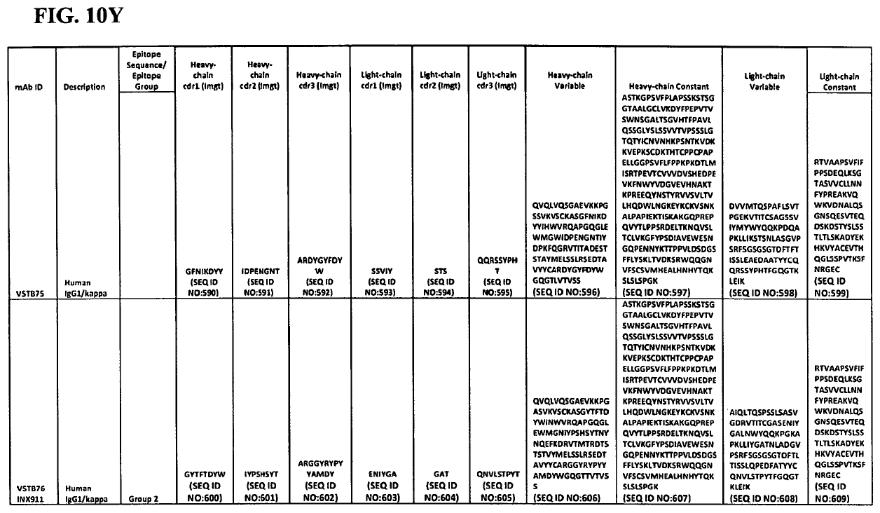

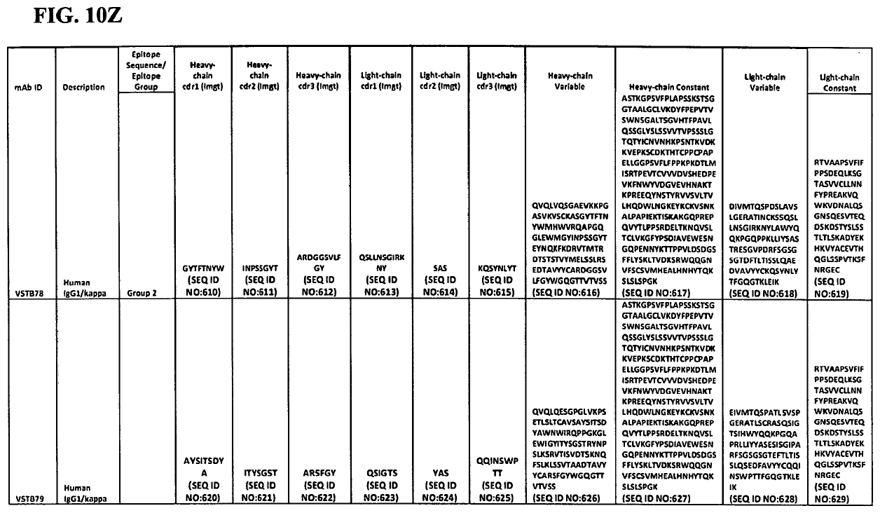

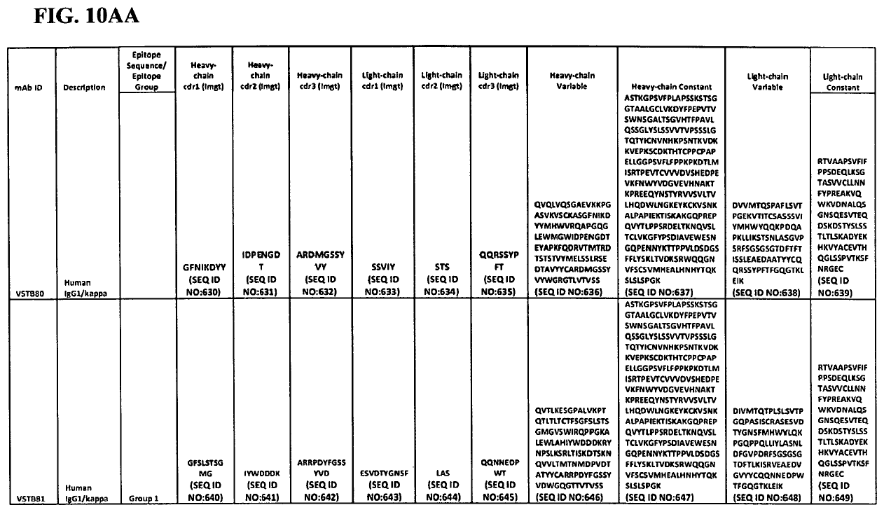

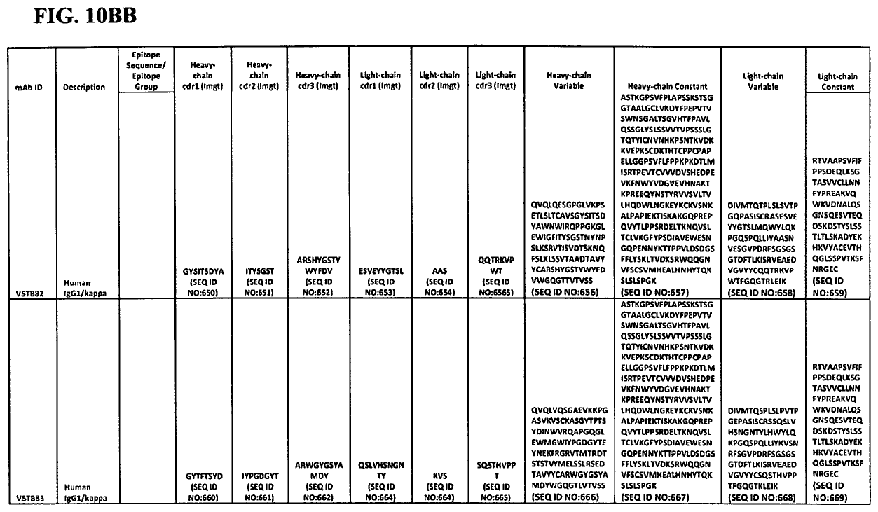

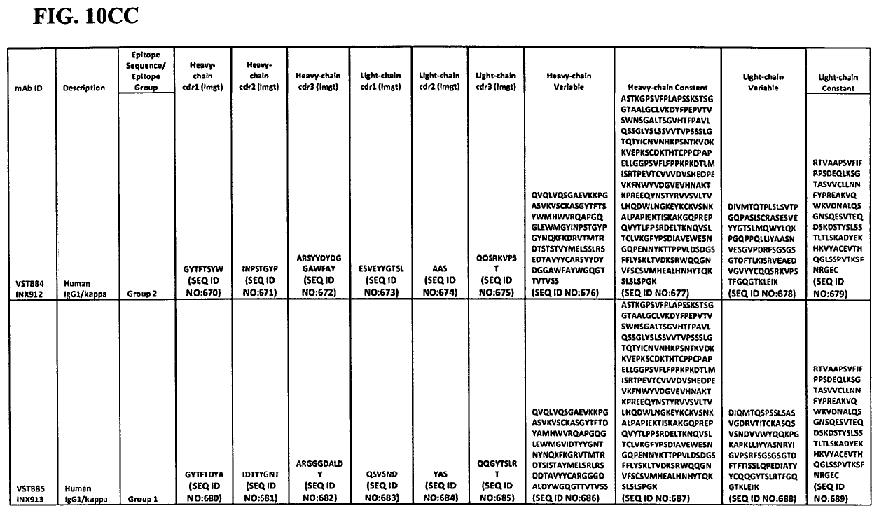

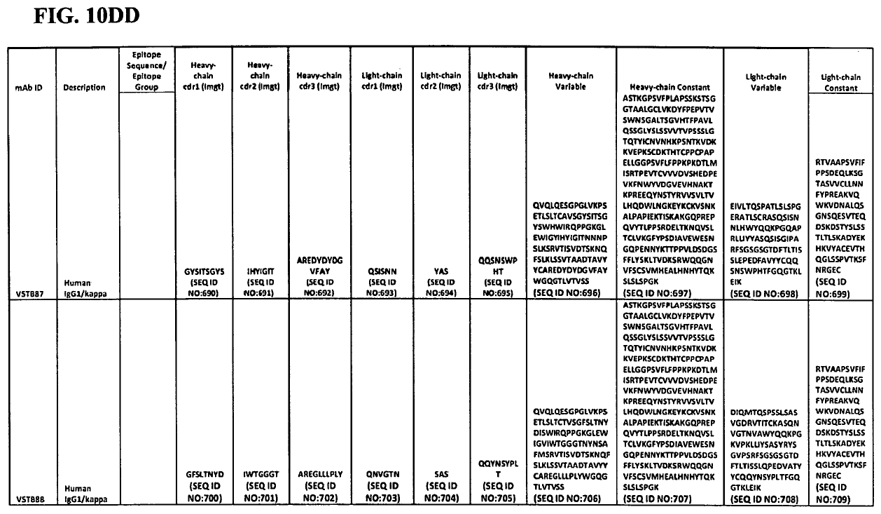

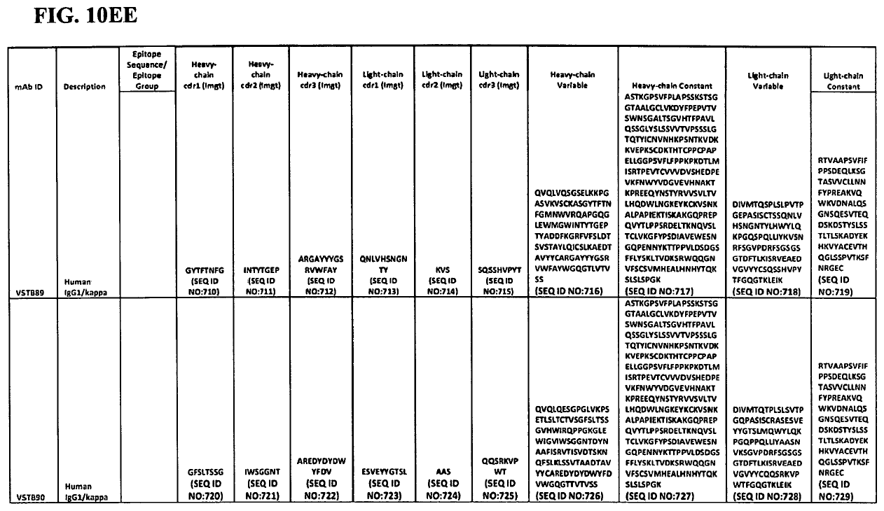

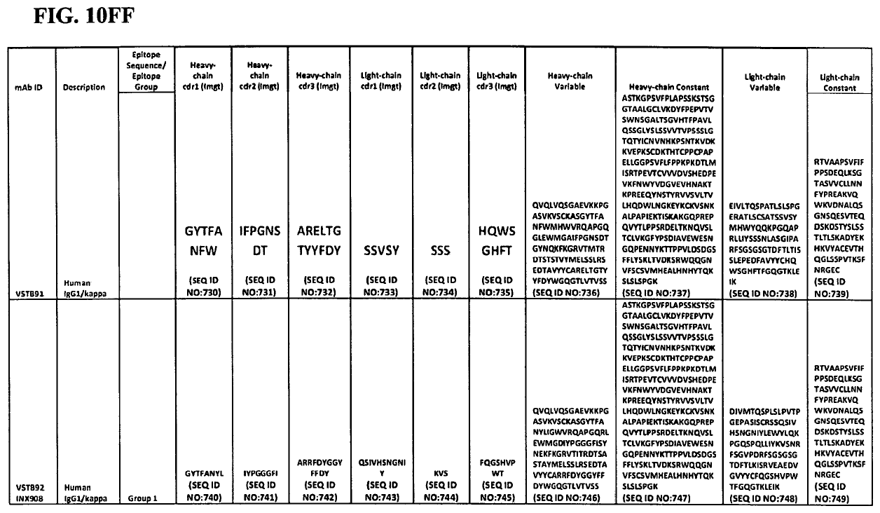

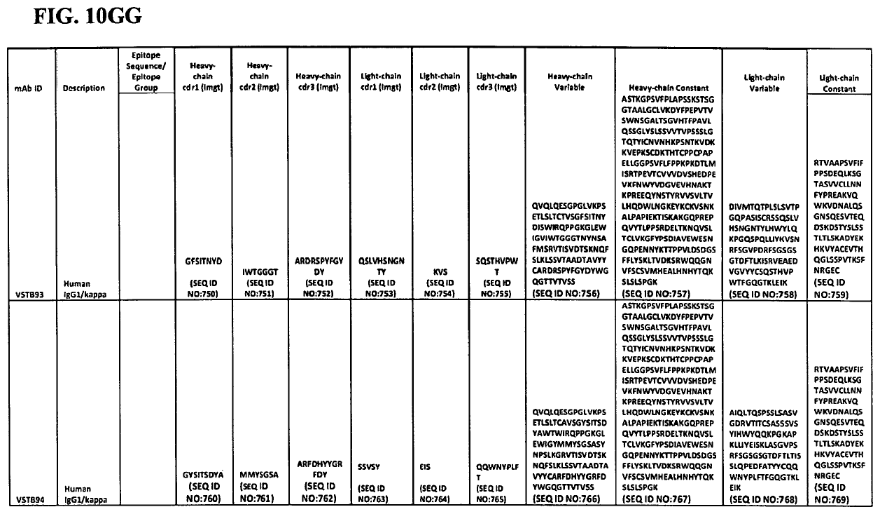

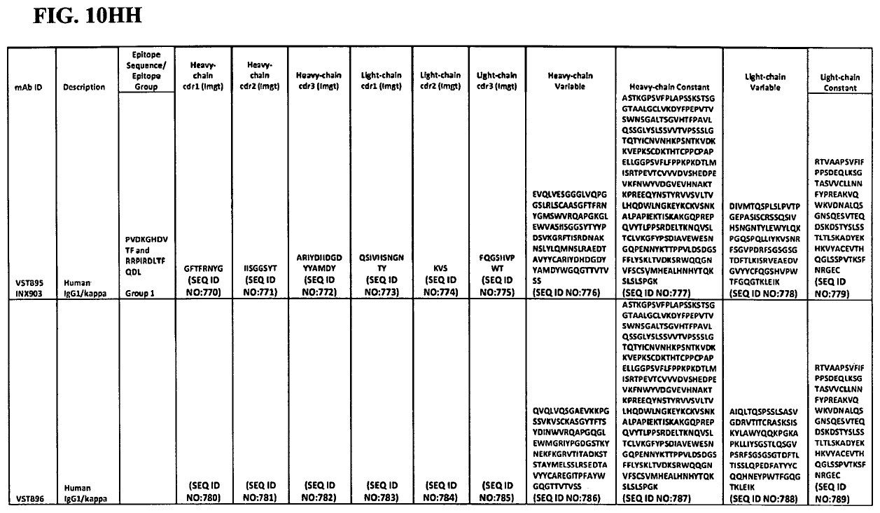

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation. Preferred epitopes on VISTA to which exemplary anti-VISTA antibodies may bind are identified in FIG. 10.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Such methods include surface plasmon resonance (BIAcore), ELISA, Kinexa Biosensor, scintillation proximity assays, ORIGEN immunoassay (IGEN), fluorescence quenching, fluorescence transfer, and/or yeast display. Binding affinity may also be screened using a suitable bioassay. In the present application the Kd of exemplary anti-VISTA antibodies comprised in exemplary ADCs was determined by surface plasmon resonance (SPR) methods on a ProteOn instrument.

“Or better” when used herein to refer to binding affinity refers to a stronger binding between a molecule and its binding partner. “Or better” when used herein refers to a stronger binding, represented by a smaller numerical Kd value. For example, an antibody which has an affinity for an antigen of “0.6 nM or better”, the antibody's affinity for the antigen is <0.6 nM, i.e. 0.59 nM, 0.58 nM, 0.57 nM etc. or any value less than 0.6 nM.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

An antibody is said to “competitively inhibit” binding of a reference antibody to a given epitope if the antibody preferentially binds to that epitope or an overlapping epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

“Isotype” herein refers to the antibody class (e.g., IgM, IgG1, IgG3, IgG3 or IgG4) that is encoded by the heavy chain constant region genes.

“K-assoc” or “Ka”, as used herein, refers broadly to the association rate of a particular antibody-antigen interaction, whereas the term “Kdiss” or “Kd,” as used herein, refers to the dissociation rate of a particular antibody-antigen interaction.

The term “KD”, as used herein, is intended to refer to the dissociation constant, which is obtained from the ratio of Kd to Ka (i. e., Kd/Ka) and is expressed as a molar concentration (M). KD values for antibodies can be determined using methods well established in the art such as plasmon resonance (BIAcore®), ELISA and KINEXA. A preferred method for determining the KD of an antibody is by using surface Plasmon resonance, preferably using a biosensor system such as a BIAcore® system or by ELISA. Typically, these methods are effected at 25° or 37° C. Antibodies for therapeutic usage generally will possess a KD when determined by surface Plasmon resonance of 50 nM or less or more typically 1 nM or less at 25° or 37° C.

The phrase “Kd” herein refers Kd is the equilibrium dissociation constant, a calculated ratio of Koff/Kon, between the antibody and its antigen. The association constant (Kon) is used to characterize how quickly the antibody binds to its target. Herein the antibody Kd was determined by surface plasmon resonance (SPR) using a Proteon instrument.

The phrase “PK” herein refers to the in vivo half-life or duration (time) that half of the amount of an antibody or antibody fragment or an antibody drug conjugate (ADC), preferably an anti-VISTA or antibody fragment according to the invention, (i.e., one comprising an anti-VISTA antibody or antibody fragment that binds to VISTA expressing cells at physiologic pH) and an anti-inflammatory agent (AI), which AI is a small molecule which requires cell internalization for efficacy (anti-inflammatory activity) and typically a steroid and more typically a glucocorticosteroid agonist of Formula I, II or III, remains in peripheral circulation in the serum. PK may be determined in vivo in a subject administered the antibody or antibody fragment or ADC, e.g., a human VISTA knock-in rodents or in a primate (e.g., human or Cynomolgus macaque). As noted infra, the anti-VISTA antibodies comprised in ADCs according to the invention typically will comprise a short PK's i.e., generally around 2.3±0.7 days in Cynomolgus macaque and typically at most≈2.5 days and more typically only a day, few hours or less in human VISTA knock-in rodents.

The phrase “PD” herein refers to the duration (time) that a dosage of an antibody or antibody drug conjugate (ADC) according to the invention, e.g., one comprising an anti-VISTA antibody or antibody fragment that binds to VISTA expressing cells at physiologic pH, and an anti-inflammatory agent (AI), which AI is a small molecule which requires cell internalization for efficacy (anti-inflammatory activity) and typically a steroid and more typically comprises a glucocorticosteroids agonist of Formula I, II or III, which upon internalization into a target cell elicits efficacy (anti-inflammatory activity). The PD for a steroid, e.g., a glucocorticosteroids agonist of Formula I, II or III, or glucocorticoid agonist-linkers and ADCs containing same as disclosed herein may be determined by different assays. For example, PD of a VISTA ADC according to the invention may be determined in vitro using VISTA expressing immune cells contacted with the ADC or may be determined in vivo in a subject administered the ADC dosage, e.g., a rodent or primate (e.g., human or Cynomolgus macaque). Additionally, because the exemplary anti-VISTA ADCs bind to different immune cells (e.g., T cells, Tregs, monocytes, macrophages, neutrophils) and further since these ADCs internalize different types of immune cells differently based on the relative expression of the antigen bound thereby on said immune cell, e.g., VISTA expression, and further because the turn-over rate of such immune cells varies, the PD values if determined in vitro using different types of immune cells, e.g., VISTA expressing immune cells will vary. Generally herein in the case of anti-VISTA ADCs the PD is represented based on the duration of anti-inflammatory activity elicited by macrophages as these cells are present in the circulation and (surprisingly) VISTA antibody comprising ADCs according to the invention have been demonstrated to elicit prolonged anti-inflammatory activity in macrophages, e.g., weeks or even a month after ADC administration. However, if the ADC targets different immune cells than VISTA, e.g., B or NK cells then the PD potentially would be determined by detecting inflammatory activity in these cells as internalization of the glucocorticoid agonist would be in these cells.

The phrase “PD/PK ratio” herein refers to the ratio of the PD and PK values of an ADC according to the invention determined in vitro or in vivo in immune cells of a particular species or in an animal model, e.g., in the case of anti-VISTA ADCs in a human VISTA knock-in rodent or in a primate (e.g., human or Cynomolgus macaque).[As shown infra, the PD/PK ratios of anti-VISTA ADCs according to the invention have been demonstrated to be surprisingly high, i.e., as high as 14:1 or 28:1 in VISTA knock-in rodents and Cyno have been demonstrated. Moreover, analogous or higher PD/PK ratios are anticipated to be obtained in humans and other non-human primates since the expression of VISTA by different immune cells in rodents and human and primates is very similar and further since drug metabolism generally occurs much quicker in rodents than in human and non-human primates. While Applicant does not wish to be bound by this theory; in the case of VISTA antibody comprising ADCs according to the invention, it is believed that the subject ADCs internalize specific types of VISTA expressing cells in very high quantities because of the high density of surface VISTA expression on these immune cells which apparently creates a “depot effect”, i.e., the depot of internalized ADCs are very slowly metabolized, thereby providing for surprisingly prolonged release of therapeutically effective (anti-inflammatory) amounts of the anti-inflammatory agent (e.g., a steroid such as a glucocorticosteroid agonist of Formula I, II or III or glucocorticosteroid agonist-linker or ADC containing same).

“Onset of efficacy” refers to the time that the efficacy of a therapeutic agent, e.g., a steroid or ADC conjugate, commences in vivo. In the present invention this can be detected in a subject administered a glucocorticosteroid agonist of Formula I, II or III or glucocorticosteroid agonist-linker or ADC conjugate according to the invention, using known in vivo assays which detect the anti-inflammatory efficacy of steroids. As disclosed infra, anti-VISTA ADCs according to the invention have been shown to have a rapid onset of efficacy, i.e., about 2 hours in human VISTA knock-in rodents.

The phrase “substantially similar,” or “substantially the same”, as used herein, denotes a sufficiently high degree of similarity between two numeric values (generally one associated with an antibody of the disclosure and the other associated with a reference/comparator antibody) such that one of skill in the art would consider the difference between the two values to be of little or no biological and/or statistical significance within the context of the biological characteristic measured by said values (e.g., Kd values). The difference between said two values can be less than about 50%, less than about 40%, less than about 30%, less than about 20%, or less than about 10% as a function of the value for the reference/comparator antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

The term “immunoconjugate,” “conjugate,” “antibody-drug conjugate,” or “ADC” as used herein refers to a compound or a derivative thereof that is linked to an anti-VISTA antibody or fragment thereof) and an anti-inflammatory agent such as a glucocorticosteroid agonist and generally a linker intervening which may be represented by a generic formula: (AI-L-Q) n-A, wherein AI=anti-inflammatory agent, generally a small-molecule glucocorticoid receptor agonist, e.g., a glucocorticosteroid agonist compound according to Formula 1, II or III as disclosed herein, L=linker, Q=heterobifunctional group, a heterotrifunctional group, or is absent, and A=an anti-VISTA antibody or VISTA binding fragment thereof that preferentially binds to human VISTA at physiologic pH and which generally possess a short pK as afore-described, and n is an integer greater than 1, optionally from 1-10. Immunoconjugates can also be defined by the generic formula in reverse order: A-(Q-L-AI)ₙ.

In the present disclosure, the term “linker” refers to any chemical moiety capable of linking an antibody or antibody fragment (e.g., antigen binding fragments) or functional equivalent to an anti-inflammatory agent drug, generally a glucocorticosteroid receptor agonist, e.g., a glucocorticosteroid agonist of Formula I, II or III. Linkers may be susceptible to cleavage (a “cleavable linker”) thereby facilitating release of the anti-inflammatory agent such as a glucocorticosteroid. For example, such cleavable linkers may be susceptible to acid-induced cleavage, photo-induced cleavage, peptidase-induced cleavage, esterase-induced cleavage, and disulfide bond cleavage, at conditions under whereby the glucocorticosteroid and/or the antibody remains active before or after internalization into an immune cell such as a neutrophil, monocyte, macrophage, eosinophil, T cell, dendritic cell, Treg, NK cell, B cell, mast cell, macrophage or myeloid cell, among other immune cell types. Alternatively, linkers may be substantially resistant to cleavage (a “noncleavable linker”).

Non-cleavable linkers include any chemical moiety capable of linking an anti-inflammatory agent such as a glucocorticosteroid agonist of Formula I, II or III to an antibody in a stable, covalent manner and does not fall off under the categories listed above for cleavable linkers. Thus, non-cleavable linkers are substantially resistant to acid-induced cleavage, photo-induced cleavage, peptidase-induced cleavage, esterase-induced cleavage and disulfide bond cleavage. Furthermore, non-cleavable refers to the ability of the chemical bond in the linker or adjoining to the linker to withstand cleavage induced by an acid, photolabile-cleaving agent, a peptidase, an esterase, or a chemical or physiological compound that cleaves a disulfide bond, at conditions under which a glucocorticosteroid and/or the antibody does not lose its activity before or after internalization into an immune cell such as a monocyte or myeloid cell.

Some cleavable linkers are cleaved by peptidases (“peptidase cleavable linkers”). Only certain peptides are readily cleaved inside or outside cells, See e.g. Trout et al., 79 Proc. Natl. Acad. Sci. USA, 626-629 (1982) and Umemoto et al. 43 Int. J. Cancer, 677-684 (1989). Furthermore, peptides are composed of α-amino acid units and peptidic bonds, which chemically are amide bonds between the carboxylate of one amino acid and the amino group of a second amino acid. Other amide bonds, such as the bond between a carboxylate and the a amino acid group of lysine, are understood not to be peptidic bonds and are considered non-cleavable.

Some linkers are cleaved by esterases (“esterase cleavable linkers”). Only certain esters can be cleaved by esterases present inside or outside of cells. Esters are formed by the condensation of a carboxylic acid and an alcohol. Simple esters are esters produced with simple alcohols, such as aliphatic alcohols, and small cyclic and small aromatic alcohols.

In some embodiments, the cleavable linker component may comprise a peptide comprising one to ten amino acid residues. In these embodiments, the peptide allows for cleavage of the linker by a protease, thereby facilitating release of the anti-inflammatory agent, e.g., glucocorticosteroid upon exposure to intracellular proteases, such as lysosomal enzymes (Doronina et al. (2003) Nat. Biotechnol. 21:778-784). Exemplary peptides include, but are not limited to, dipeptides, tripeptides, tetrapeptides, and pentapeptides. Exemplary dipeptides include, but are not limited to, alanine-alanine (ala-ala), valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly) as well as the specific linkers identified in the “Exemplary Embodiments” section and embodied in Example 3 of this application.

A peptide may comprise naturally-occurring and/or non-natural amino acid residues. The term “naturally-occurring amino acid” refer to Ala, Asp, Cys, Glu, Phe, Gly, His, He, Lys, Leu, Met, Asn, Pro, Gin, Arg, Ser, Thr, Val, Trp, and Tyr. “Non-natural amino acids” (i.e., amino acids do not occur naturally) include, by way of non-limiting example, homoserine, homoarginine, citrulline, phenylglycine, taurine, iodotyrosine, seleno-cysteine, norleucine (“Nle”), norvaline (“Nva”), beta-alanine, L- or D-naphthalanine, ornithine (“Orn”), and the like. Peptides can be designed and optimized for enzymatic cleavage by a particular enzyme, for example, a tumor-associated protease, cathepsin B, C and D, or a plasmin protease.

Amino acids also include the D-forms of natural and non-natural amino acids. “D-” designates an amino acid having the “D” (dextrorotary) configuration, as opposed to the configuration in the naturally occurring (“L-”) amino acids. Natural and non-natural amino acids can be purchased commercially (Sigma Chemical Co., Advanced Chemtech) or synthesized using methods known in the art.

The term “drug antibody ratio” or “DAR” refers to the number of anti-inflammatory agent or functional derivative (i.e., radical derived from a small-molecule glucocorticoid receptor agonist, e.g., a glucocorticosteroid of Formula I, II or III). Thus, in the immunoconjugate having the generic formula (AI-L-Q) n-A or the reverse, the DAR is defined by the variable “n.” Typically in the subject ADCs “n” ranges from 1-12.

When referring to a compound having formula (AI-L-Q) n-A representing an individual immunoconjugate, the DAR refers to the number of inflammatory agent or functional derivative (e.g., radical derived from a small-molecule glucocorticoid receptor agonist, e.g., a glucocorticosteroid such as dexamethasone or Budesonide or a novel glucocorticosteroid of Formula I, II or III which are linked to the A (e.g., n optionally is an integer or fraction of 1 to 12). linked to a particular A (e.g., n is an integer of 1 to 12).

When referring to a compound having formula (AI-L-Q) n-A representing a plurality of immunoconjugates, the DAR refers to the average number of anti-inflammatory agents or functional derivatives (e.g., radical derived from a small-molecule glucocorticoid receptor agonist, e.g., a glucocorticosteroid such as a novel steroid of Formula I, II or III which are linked to the A (e.g., n is an integer or fraction of 1 to 12) by a linker. Thus, by way of an example, a compound having formula (AI-L-Q) n-A comprising a first immunoconjugate with 3 AI per A and a second immunoconjugate with 4 AI per A would have a DAR (i.e., an “n”) of 3.5.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

An “effective amount” of an ADC or glucocorticoid receptor agonist as disclosed herein is an amount sufficient to carry out a specifically stated purpose. An “effective amount” can be determined in relation to the stated purpose.

The term “therapeutically effective amount” refers to an amount of an immunoconjugate or glucocorticoid receptor agonist effective to “treat” a disease or disorder in a subject or mammal. A “prophylactically effective amount” refers to an amount effective to achieve the desired prophylactic result.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. Prophylactic or preventative measures refer to measures that prevent and/or slow the development of a targeted pathological condition or disorder. Thus, those in need of prophylactic or preventative measures include those prone to have the disorder and those in whom the disorder is to be prevented.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. If present, modification to the nucleotide structure can be imparted before or after assembly of the polymer. The sequence of nucleotides can be interrupted by non-nucleotide components. A polynucleotide can be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced, for example, by phosphonate groups, phosphate groups, protected by standard protecting groups, or activated to prepare additional linkages to additional nucleotides, or can be conjugated to solid supports. The 5′ and 3′ terminal OH can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages can be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “vector” means a construct, which is capable of delivering, and optionally expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in certain embodiments, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, Proc. Natl. Acad. Sci., 87:2264-2268 (1990), as modified in Karlin et al., Proc. Natl. Acad. Sci., 90:5873-5877 (1993), and incorporated into the NBLAST and XBLAST programs (Altschul et al., Nucleic Acids Res., 25:3389-3402 (1991)). In certain embodiments, Gapped BLAST can be used as described in Altschul et al., Nucleic Acids Res. 25:3389-3402 (1997). BLAST-2, WU-BLAST-2 (Altschul et al., Methods in Enzymology, 266:460-480 (1996)), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48): 444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100 times (Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman (Advances in Applied Mathematics 2:482 489 (1981)) to find the best segment of homology between two sequences. When using Bestfit or any other sequence alignment program to determine whether a particular sequence is, for instance, 95% identical to a reference sequence according to the present disclosure, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value there between, and can be over a longer region than 60-80 residues, for example, at least about 90-100 residues, and in some embodiments, the sequences are substantially identical over the full length of the sequences being compared, such as the coding region of a nucleotide sequence for example.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In some embodiments, conservative substitutions in the sequences of the polypeptides and antibodies of the disclosure do not abrogate the binding of the antibody containing the amino acid sequence, to the antigen(s), e.g., the VISTA to which the antibody binds. Methods of identifying nucleotide and amino acid conservative substitutions which do not eliminate antigen binding are well-known in the art (see, e.g., Brummell et al., Biochem. 32:1180-1 187 (1993); Kobayashi et al., Protein Eng. 12 (10): 879-884 (1999); and Burks et al., Proc. Natl. Acad. Sci. USA 94:412-417 (1997)).

As used herein, “substantially pure” refers to material which is at least 50% pure (i.e., free from contaminants), more preferably at least 90% pure, more preferably at least 95% pure, more preferably at least 98% pure, more preferably at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3.

As used herein, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, ImmunoMethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202:163 (1996), may be performed.

A “functional Fc region” possesses at least one effector function of a native sequence Fc region. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” or “endogenous FcR” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95%, at least about 96%, at least about 97%, at least about 98%, at least about 99% sequence identity therewith.

As used herein “antibody-dependent cell-mediated cytotoxicity” and “ADCC” refer to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMCS) and NK cells. Alternatively, or additionally, ADCC activity of the molecule of interest may be assessed in vivo, e.g., in an animal model such as that disclosed in Clynes et al., 1998, PNAS (USA), 95:652-656.

In the present disclosure, the term “halo” as used by itself or as part of another group refers to —CI, —F, —Br, or —I. For example, the halo is —Cl or —F.

In the present disclosure, the term “hydroxy” as used by itself or as part of another group refers to —OH.

In the present disclosure, the term “thiol” or the term “sulfhydryl” as used by itself or as part of another group refers to —SH.

In the present disclosure, the term “alkyl” as used by itself or as part of another group refers to unsubstituted straight- or branched-chain aliphatic hydrocarbons containing from one to twelve carbon atoms, i.e., C₁₋₁₂ alkyl, or the number of carbon atoms designated, e.g., a C₁ alkyl such as methyl, a C₂ alkyl such as ethyl, a C₃ alkyl such as propyl or isopropyl, a C₁₋₃ alkyl such as methyl, ethyl, propyl, or isopropyl, and so on. For example, the alkyl is a C₁₋₁₀ alkyl. In another example, the alkyl is a C₁₋₆ alkyl. In another example, the alkyl is a C₁₋₄ alkyl. In another example, the alkyl is a straight chain C1-10 alkyl. In another example, the alkyl is a branched chain C₃₋₁₀ alkyl. In another example, the alkyl is a straight chain C₁₋₆ alkyl. In another example, the alkyl is a branched chain C₃₋₆ alkyl. In another example, the alkyl is a straight chain C₁₋₄ alkyl. In another example, the alkyl is a branched chain C₃₋₄ alkyl. In another example, the alkyl is a straight or branched chain C₃₋₄ alkyl. Non-limiting exemplary C₁₋₁₀ alkyl groups include methyl, ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tert-butyl, and iso-butyl.

In the present disclosure, the term “optionally substituted alkyl” as used by itself or as part of another group refers to an alkyl that is either unsubstituted or substituted with one, two, or three substituents independently selected from the group consisting of nitro, hydroxy, cyano, haloalkoxy, aryloxy, alkylthio, sulfonamido, alkylcarbonyl, arylcarbonyl, alkylsulfonyl, arylsulfonyl, carboxy, carboxamido, alkoxycarbonyl, thiol, —N(H)C(═O) NH₂, and —N(H)C═NH) NH2, optionally substituted aryl, and optionally substituted heteroaryl. For instance, the optionally substituted alkyl is substituted with two substituents. In another example, the optionally substituted alkyl is substituted with one substituent. In another example, the optionally substituted alkyl is unsubstituted. Non-limiting exemplary substituted alkyl groups include —CH₂OH, —CH₂SH, —CH₂Ph, —CH₂ (4-OH)Ph, —CH₂ (imidazolyl), —CH₂CH₂CO₂H, —CH₂CH₂SO₂CH₃, —CH₂CH₂COPh, and —CH₂OC(═O)CH₃.

In the present disclosure, the term “cycloalkyl” as used by itself or as part of another group refers to unsubstituted saturated or partially unsaturated, e.g., containing one or two double bonds, cyclic aliphatic hydrocarbons containing one to three rings having from three to twelve carbon atoms, i.e., C₃₋₁₂ cycloalkyl, or the number of carbons designated. In one example, the cycloalkyl has two rings. In another example, the cycloalkyl has one ring. In another example, the cycloalkyl is saturated. In another example, the cycloalkyl is unsaturated. In another example, the cycloalkyl is a C₃₋₈ cycloalkyl. In another example, the cycloalkyl is a C₃₋₆ cycloalkyl. The term “cycloalkyl” is meant to include groups wherein a ring —CH₂— is replaced with a —C(═O)—. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, cyclopentenyl, and cyclopentanone.

In the present disclosure, the term “optionally substituted cycloalkyl” as used by itself or as part of another group refers to a cycloalkyl that is either unsubstituted or substituted with one, two, or three substituents independently selected from the group consisting of halo, nitro, cyano, hydroxy, alkylcarbonyloxy, cycloalkylcarbonyloxy, amino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, (heterocyclo)alkyl, and —OC(═O)-amino, The term optionally substituted cycloalkyl includes cycloalkyl groups having a fused optionally substituted aryl, e.g., phenyl, or fused optionally substituted heteroaryl, e.g., pyridyl. An optionally substituted cycloalkyl having a fused optionally substituted aryl or fused optionally substituted heteroaryl group may be attached to the remainder of the molecule at any available carbon atom on the cycloalkyl ring. In one example, the optionally substituted cycloalkyl is substituted with two substituents. In another example, the optionally substituted cycloalkyl is substituted with one substituent. In another example, the optionally substituted cycloalkyl is unsubstituted.

In the present disclosure, the term “aryl” as used by itself or as part of another group refers to unsubstituted monocyclic or bicyclic aromatic ring systems having from six to fourteen carbon atoms, i.e., a C₆₋₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one example, the aryl group is phenyl or naphthyl.

In the present disclosure, the term “optionally substituted aryl” as used herein by itself or as part of another group refers to an aryl that is either unsubstituted or substituted with one to five substituents independently selected from the group consisting of halo, nitro, cyano, hydroxy, thiol, amino, alkylamino, dialkylamino, optionally substituted alkyl, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, haloalkylsulfonyl cycloalkylsulfonyl, (cycloalkyl)alkylsulfonyl, arylsulfonyl, heteroarylsulfonyl, heterocyclosulfonyl, carboxy, carboxyalkyl, optionally substituted cycloalkyl, alkenyl, alkynyl, optionally substituted aryl, optionally substituted heteroaryl, optionally substituted heterocyclo, alkoxycarbonyl, alkoxyalkyl, (amino)alkyl, (carboxamido)alkyl, and (heterocyclo)alkyl.

In one example, the optionally substituted aryl is an optionally substituted phenyl. In another example, the optionally substituted phenyl has four substituents. In another example, the optionally substituted phenyl has three substituents. In another example, the optionally substituted phenyl has two substituents. In another example, the optionally substituted phenyl has one substituent. In another example, the optionally substituted phenyl is unsubstituted. Non-limiting exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, 4-(pyridin-4-ylsulfonyl) phenyl The term optionally substituted aryl includes phenyl groups having a fused optionally substituted cycloalkyl or fused optionally substituted heterocyclo group. An optionally substituted phenyl having a fused optionally substituted cycloalkyl or fused optionally substituted heterocyclo group may be attached to the remainder of the molecule at any available carbon atom on the phenyl ring.

In the present disclosure, the term “alkenyl” as used by itself or as part of another group refers to an alkyl containing one, two or three carbon-to-carbon double bonds. In one example, the alkenyl has one carbon-to-carbon double bond. In another example, the alkenyl is a C2-6 alkenyl. In another example, the alkenyl is a C2-4 alkenyl. Non-limiting exemplary alkenyl groups include ethenyl, propenyl, isopropenyl, butenyl, sec-butenyl, pentenyl, and hexenyl.

In the present disclosure, the term “optionally substituted alkenyl” as used herein by itself or as part of another group refers to an alkenyl that is either unsubstituted or substituted with one, two or three substituents independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, optionally substituted alkyl, optionally substituted cycloalkyl, alkenyl, alkynyl, optionally substituted aryl, heteroaryl, and optionally substituted heterocyclo.

In the present disclosure, the term “alkynyl” as used by itself or as part of another group refers to an alkyl containing one to three carbon-to-carbon triple bonds. In one example, the alkynyl has one carbon-to-carbon triple bond. In another example, the alkynyl is a C2-6 alkynyl. In another example, the alkynyl is a C2-4 alkynyl. Non-limiting exemplary alkynyl groups include ethynyl, propynyl, butynyl, 2-butynyl, pentynyl, and hexynyl groups.

In the present disclosure, the term “optionally substituted alkynyl” as used herein by itself or as part refers to an alkynyl that is either unsubstituted or substituted with one, two or three substituents independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, optionally substituted alkyl, cycloalkyl, alkenyl, alkynyl, optionally substituted aryl, optionally substituted heteroaryl, and heterocyclo.

In the present disclosure, the term “haloalkyl” as used by itself or as part of another group refers to an alkyl substituted by one or more fluorine, chlorine, bromine and/or iodine atoms. In one example, the alkyl group is substituted by one, two, or three fluorine and/or chlorine atoms. In another example, the haloalkyl group is a C₁₋₄ haloalkyl group. Non-limiting exemplary haloalkyl groups include fluoromethyl, 2-fluoroethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

In the present disclosure, the term “alkoxy” as used by itself or as part of another group refers to an optionally substituted alkyl, optionally substituted cycloalkyl, optionally substituted alkenyl, or optionally substituted alkynyl attached to a terminal oxygen atom. In one example, the alkoxy is an optionally substituted alkyl attached to a terminal oxygen atom. In one example, the alkoxy group is a C₁₋₆ alkyl attached to a terminal oxygen atom. In another example, the alkoxy group is a C₁₋₄ alkyl attached to a terminal oxygen atom. Non-limiting exemplary alkoxy groups include methoxy, ethoxy, and tert-butoxy.

In the present disclosure, the term “alkylthio” as used by itself or as part of another group refers to an optionally substituted alkyl attached to a terminal sulfur atom. In one example, the alkylthio group is a C₁₋₄ alkylthio group. Non-limiting exemplary alkylthio groups include —SCH₃ and —SCH₂CH₃.

In the present disclosure, the term “haloalkoxy” as used by itself or as part of another group refers to a haloalkyl attached to a terminal oxygen atom. Non-limiting exemplary haloalkoxy groups include fluoromethoxy, difluoromethoxy, trifluoromethoxy, and 2,2,2-trifluoroethoxy.

In the present disclosure, the term “heteroaryl” refers to unsubstituted monocyclic and bicyclic aromatic ring systems having 5 to 14 ring atoms, i.e., a 5- to 14-membered heteroaryl, wherein at least one carbon atom of one of the rings is replaced with a heteroatom independently selected from the group consisting of oxygen, nitrogen and sulfur. In one example, the heteroaryl contains 1, 2, 3, or 4 heteroatoms independently selected from the group consisting of oxygen, nitrogen and sulfur. In one example, the heteroaryl has three heteroatoms. In another example, the heteroaryl has two heteroatoms. In another example, the heteroaryl has one heteroatom. In another example, the heteroaryl is a 5- to 10-membered heteroaryl. In another example, the heteroaryl is a 5- or 6-membered heteroaryl. In another example, the heteroaryl has 5 ring atoms, e.g., thienyl, a 5-membered heteroaryl having four carbon atoms and one sulfur atom. In another example, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl,β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one example, the heteroaryl is selected from the group consisting of thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl), isoxazolyl (e.g., isoxazol-3-yl, isoxazol-4-yl, and isoxazol-5-yl), and indazolyl (e.g., 1H-indazol-3-yl). The term “heteroaryl” is also meant to include possible N-oxides. A non-limiting exemplary N-oxide is pyridyl N-oxide.

In one example, the heteroaryl is a 5- or 6-membered heteroaryl. In one example, the heteroaryl is a 5-membered heteroaryl, i.e., the heteroaryl is a monocyclic aromatic ring system having 5 ring atoms wherein at least one carbon atom of the ring is replaced with a heteroatom independently selected from nitrogen, oxygen, and sulfur. Non-limiting exemplary 5-membered heteroaryl groups include thienyl, furyl, pyrrolyl, oxazolyl, pyrazolyl, imidazolyl, thiazolyl, isothiazolyl, and isoxazolyl. In another example, the heteroaryl is a 6-membered heteroaryl, e.g., the heteroaryl is a monocyclic aromatic ring system having 6 ring atoms wherein at least one carbon atom of the ring is replaced with a nitrogen atom. Non-limiting exemplary 6-membered heteroaryl groups include pyridyl, pyrazinyl, pyrimidinyl, and pyridazinyl.

In the present disclosure, the term “optionally substituted heteroaryl” as used by itself or as part of another group refers to a heteroaryl that is either unsubstituted or substituted with one two, three, or four substituents, independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, haloalkylsulfonyl cycloalkylsulfonyl, (cycloalkyl)alkylsulfonyl, arylsulfonyl, heteroarylsulfonyl, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, and (heterocyclo)alkyl. In one example, the optionally substituted heteroaryl has one substituent. In another example, the optionally substituted heteroaryl is unsubstituted. Any available carbon or nitrogen atom can be substituted. The term optionally substituted heteroaryl includes heteroaryl groups having a fused optionally substituted cycloalkyl or fused optionally substituted heterocyclo group. An optionally substituted heteroaryl having a fused optionally substituted cycloalkyl or fused optionally substituted heterocyclo group may be attached to the remainder of the molecule at any available carbon atom on the heteroaryl ring.

In the present disclosure, the term “heterocyclo” as used by itself or as part of another group refers to unsubstituted saturated and partially unsaturated, e.g., containing one or two double bonds, cyclic groups containing one, two, or three rings having from three to fourteen ring members, i.e., a 3- to 14-membered heterocyclo, wherein at least one carbon atom of one of the rings is replaced with a heteroatom. Each heteroatom is independently selected from the group consisting of oxygen, sulfur, including sulfoxide and sulfone, and/or nitrogen atoms, which can be oxidized or quaternized. The term “heterocyclo” includes groups wherein a ring —CH₂— is replaced with a —C(═O)—, for example, cyclic ureido groups such as 2-imidazolidinone and cyclic amide groups such as β-lactam, γ-lactam, δ-lactam, ε-lactam, and piperazin-2-one. The term “heterocyclo” also includes groups having fused optionally substituted aryl groups, e.g., indolinyl or chroman-4-yl. In one embodiment, the heterocyclo group is a C₄₋₆ heterocyclo, i.e., a 4-, 5- or 6-membered cyclic group, containing one ring and one or two oxygen and/or nitrogen atoms. In one embodiment, the heterocyclo group is a C₄₋₆ heterocyclo containing one ring and one nitrogen atom. The heterocyclo can be optionally linked to the rest of the molecule through any available carbon or nitrogen atom. Non-limiting exemplary heterocyclo groups include azetidinyl, dioxanyl, tetrahydropyranyl, 2-oxopyrrolidin-3-yl, piperazin-2-one, piperazine-2,6-dione, 2-imidazolidinone, piperidinyl, morpholinyl, piperazinyl, pyrrolidinyl, and indolinyl.

In the present disclosure, the term “optionally substituted heterocyclo” as used herein by itself or part of another group refers to a heterocyclo that is either unsubstituted or substituted with one, two, three, or four substituents independently selected from the group consisting of halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, cycloalkylcarbonyl, alkoxycarbonyl, CF₃C(═O)—, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, or (heterocyclo)alkyl. Substitution may occur on any available carbon or nitrogen atom, or both.

In the present disclosure, the term “amino” as used by itself or as part of another group refers to a radical of the formula —NRO^{a}R^{b}, wherein R^{a} and R^{b} are each independently selected from the group consisting of hydrogen, optionally substituted alkyl, and aralkyl, or R^{a} and R^{b} are taken together to form a 3- to 8-membered optionally substituted heterocyclo. Non-limiting exemplary amino groups include —NH2 and —N(H)(CH₃).

In the present disclosure, the term “carboxamido” as used by itself or as part of another group refers to a radical of formula —C(═O)NR^{a}R^{b}, wherein R^{a} and R^{b} are each independently selected from the group consisting of hydrogen, optionally substituted alkyl, hydroxyalkyl, and optionally substituted aryl, optionally substituted heterocyclo, and optionally substituted heteroaryl, or R^{a} and R^{b} taken together with the nitrogen to which they are attached form a 3- to 8-membered optionally substituted heterocyclo group. In one embodiment, R^{a} and R^{b} are each independently hydrogen or optionally substituted alkyl. In one embodiment, R^{a} and R^{b} are taken together to taken together with the nitrogen to which they are attached form a 3- to 8-membered optionally substituted heterocyclo group. Non-limiting exemplary carboxamido groups include —CONH₂, —CON(H)CH₃, and —CON(CH₃)₂.

In the present disclosure, the term “alkoxycarbonyl” as used by itself or as part of another group refers to a carbonyl group, i.e., —C(═O)—, substituted with an alkoxy. In one embodiment, the alkoxy is a C₁₋₄ alkoxy. Non-limiting exemplary alkoxycarbonyl groups include —C(═O)OMe, —C(═O)OEt, and —C(═O)OtBu.

In the present disclosure, the term “carboxy” as used by itself or as part of another group refers to a radical of the formula —CO₂H.

In the present disclosure, the term “self-immolative group” or “immolative group” or “immolative linker” refers to all or part of a cleavable linker and comprises a bifunctional chemical moiety that is capable of covalently linking two spaced chemical moieties into a normally stable tripartite molecule, can release one of the spaced chemical moieties from the tripartite molecule by means of enzymatic cleavage; and following enzymatic cleavage, can spontaneously cleave from the remainder of the molecule to release the other of the spaced chemical moieties, e.g., a glucocorticosteroid of Formula I, II or III. In some embodiments, an immolative linker comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the immolative linker is p-aminobenzyloxycarbonyl (PAB). (See Example 3 and Exemplary Embodiments section of this application).

In the present disclosure, the term “protecting group” or “PG” refers to a group that blocks, i.e., protects, a functionality, e.g., an amine functionality while reactions are carried out on other functional groups or parts of the molecule. Those skilled in the art will be familiar with the selection, attachment, and cleavage of amine protecting groups, and will appreciate that many different protective groups are known in the art, the suitability of one protective group or another being dependent on the particular the synthetic scheme planned. Treatises on the subject are available for consultation, such as Wuts, P. G. M.; Greene, T. W., “Greene's Protective Groups in Organic Synthesis”, 4th Ed., J. Wiley & Sons, N Y, 2007. Suitable protecting groups include the carbobenzyloxy (Cbz), tert-butyloxycarbonyl (BOC), 9-fluorenylmethyloxycarbonyl (FMOC), and benzyl (Bn) group. In one embodiment, the protecting group is the BOC group.

In the present disclosure, the term “ethylene glycol” refers to a chemical of the formula —OCH2CH2O—.

In the present disclosure, the term “ethylene oxide” refers to a chemical of the formula —CH2CH2O—.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

“Autoimmunity” or “autoimmune disease or condition,” as used herein, refers broadly to a disease or disorder arising from and directed against an individual's own tissues or a co-segregate or manifestation thereof or resulting condition therefrom, and includes. Herein autoimmune conditions include inflammatory or allergic conditions, e.g., chronic diseases characterized by a host immune reaction against self-antigens potentially associated with tissue destruction such as rheumatoid arthritis characterized by inflammation and/or wherein steroids are an effective treatment.

“Allergic disease or condition” or “allergic reaction” are conditions caused by hypersensitivity of the immune system to typically harmless substances or antigens in the environment. These diseases include by way of example atopic dermatitis, allergic asthma, primary immunodeficiency, chronic sinusitis, eosinophil-associated diseases and other conditions involving allergic responses or reactions.

“Immune cell,” as used herein, refers broadly to cells that are of hematopoietic origin and that play a role in the immune response. Immune cells include but are not limited to lymphocytes, such as B cells and T cells; natural killer cells; dendritic cells, and myeloid cells, such as monocytes, macrophages, eosinophils, mast cells, basophils, and granulocytes, among other immune cell types.

“Immune related disease (or disorder or condition)” as used herein should be understood to encompass any disease disorder or condition selected from the group including but not limited to autoimmune diseases, inflammatory disorders and immune disorders associated with graft transplantation rejection, such as acute and chronic rejection of organ transplantation, allogenic stem cell transplantation, autologous stem cell transplantation, bone marrow transplantation, and graft versus host disease.

“Inflammatory disorders”, “inflammatory conditions” and/or “inflammation”, used interchangeably herein, refers broadly to chronic or acute inflammatory diseases, and expressly includes inflammatory autoimmune diseases and inflammatory allergic conditions. These conditions include by way of example inflammatory abnormalities characterized by dysregulated immune response to harmful stimuli, such as pathogens, damaged cells, or irritants. Inflammatory disorders underlie a vast variety of human diseases. Non-immune diseases with etiological origins in inflammatory processes include cancer, atherosclerosis, and ischemic heart disease. Examples of disorders associated with inflammation include: Chronic prostatitis, Glomerulonephritis, Hypersensitivities, Pelvic inflammatory disease, Reperfusion injury, Sarcoidosis, Vasculitis, Interstitial cystitis, normocomplementemic urticarial vasculitis, pericarditis, myositis, anti-synthetase syndrome, scleritis, macrophage activation syndrome, Behçet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, TNF receptor-associated periodic syndrome (TRAPSP), gingivitis, periodontitis, hepatitis, cirrhosis, pancreatitis, myocarditis, vasculitis, gastritis, gout, gouty arthritis, and inflammatory skin disorders, selected from the group consisting of psoriasis, atopic dermatitis, eczema, rosacea, urticaria, and acne.

“Mammal,” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, camels, chimpanzees, chinchillas, cattle, dogs, goats, gorillas, hamsters, horses, humans, lemurs, llamas, mice, non-human primates, pigs, rats, sheep, shrews, squirrels, tapirs, and voles. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D. C.

“Patient,” or “subject” or “recipient”, “individual”, or “treated individual” are used interchangeably herein, and refers broadly to any animal that needs treatment either to alleviate a disease state or to prevent the occurrence or reoccurrence of a disease state. Also, “Patient” as used herein, refers broadly to any animal that has risk factors, a history of disease, susceptibility, symptoms, and signs, was previously diagnosed, is at risk for, or is a member of a patient population for a disease. The patient may be a clinical patient such as a human or a veterinary patient such as a companion, domesticated, livestock, exotic, or zoo animal.

“Subject” or “patient” or “individual” in the context of therapy or diagnosis herein includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc., i.e., anyone suitable to be treated according to the present invention include, but are not limited to, avian and mammalian subjects, and are preferably mammalian. Any mammalian subject in need of being treated according to the present invention is suitable. Human subjects of both genders and at any stage of development (i. e., neonate, infant, juvenile, adolescent, and adult) can be treated according to the present invention. The present invention may also be carried out on animal subjects, particularly mammalian subjects such as mice, rats, dogs, cats, cattle, goats, sheep, and horses for veterinary purposes, and for drug screening and drug development purposes. “Subjects” is used interchangeably with “individuals” and “patients.”

“Therapy,” “therapeutic,” “treating,” or “treatment”, as used herein, refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms (e.g., inflammation, pain). Therapy also encompasses “prophylaxis”. The term “reduced”, for purpose of therapy, refers broadly to the clinically significant reduction in signs and/or symptoms. Therapy includes treating relapses or recurrent signs and/or symptoms (e.g., inflammation, pain). Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms (e.g., inflammation, pain).

Having defined certain terms and phrases used in the present application, the novel glucocorticosteroid steroid agonists, glucocorticosteroid steroid agonist-linkers and ADCs containing same, methods for the production and use thereof according to the invention are further described below.

The present invention relates to ADCs comprising a novel glucocorticosteroid agonist of Formula I, II or III directly or indirectly attached via a linker to an antibody or antibody fragment comprising an antigen binding region that binds to an immune cell antigen, typically a human immune cell antigen, e.g., human V-domain Ig Suppressor of T cell Activation (VISTA). However, it is shown herein that ADCs comprising glucocorticosteroid steroid agonists and glucocorticosteroid steroid agonist-linkers according to the invention are also efficacious when coupled to an antibody or fragment that binds to immune cell antigens other than VISTA.

In some exemplary embodiments the ADCs comprise an antibody or fragment possesses a short serum half-life under physiological pH conditions (≈pH 7.5), e.g., wherein the serum half-life of the antibody or fragment in a rodent (human VISTA knock-in) generally is 1 to 72 hours, 1 to 32 hours, 1 to 16 hours, 1 to 8 hours, 1 to 4 hours or 1-2 hours±0.5 hour in a human VISTA knock-in rodent or ≈3.5, 3, 2.5, or 2.3 days±0.5 days in a primate (Cynomolgus macaque) at physiological conditions (≈pH 7.5), which anti-human VISTA antibody or antibody fragment is directly attached or indirectly via a linker to an anti-inflammatory agent, e.g. a steroid or corticosteroid receptor agonist of Formula I, II or III or corticosteroid receptor agonist-linker containing same as disclosed herein or a functional derivative or radical thereof, i.e., a derivative which when released from an ADC containing upon internalization into an immune cell elicits the desired anti-inflammatory effect when administered to a subject, e.g., human or other mammal.

Particularly in the case of ADCs comprising an anti-VISTA antibody or antibody fragment the ADCs will specifically bind to VISTA expressing immune cells at physiologic pH and the corticosteroid receptor agonist of Formula I, II or III will be released from the ADC upon internalization into target (immune) cells such as neutrophils, monocytes such as myeloid cells, macrophages, T cells, CD4 T cells. CD8 T cells, Tregs, and other immune cells present in peripheral blood. This release of the corticosteroid receptor agonist is apparently elicited by enzymes, e.g., esterases, which provide for cleavage of the ADC after it is internalized by the target immune cells. The release of the corticosteroid receptor agonist from an ADC containing upon internalization into an immune cell then selectively elicits the desired anti-inflammatory effect in immune cells which express the antigen bound by the ADC, e.g., VISTA. As noted previously, efficacy (anti-inflammatory activity) of the corticosteroid receptor agonist is only attained after such steroid compound is internalized by a cell, e.g., an immune cell which expresses VISTA or other antigen bound by the ADC.

In preferred embodiments the antibody or fragment, e.g., an anti-VISTA antibody or fragment will comprise an Fc region that is silent, i.e., mutated to impair FcR binding, e.g., a silent IgG1, IgG2, IgG3 or IgG4, most typically a silent IgG2 or silent IgG1 or the antibody or fragment may lack an Fc region or comprise an Fc fragment which does not bind to FcRs. Exemplary silent Fc regions are disclosed infra. Thereby the ADC comprising the antibody or fragment which binds to an immune cell antigen, e.g., anti-VISTA antibody or fragment while binding to and being internalized into antigen expressing immune cells in some instances will not elicit a modulatory effect on the antigen bound thereby, e.g., VISTA, i.e., it will not agonize or antagonize the effects of the antigen it binds, e.g., it will not agonize or antagonize the suppressive effects of VISTA on immunity. Rather the therapeutic effects elicited by the ADC will be solely or predominantly attributable to the anti-inflammatory agent(s) bound thereto, i.e., a corticosteroid receptor agonist of Formula I, II or III which when comprised in an ADC upon administration is internalized and released into an immune cell and elicits the desired anti-inflammatory effect, only or preferentially in target immune cells which express the antigen bound by the ADC.

Because the subject ADCs, e.g., anti-VISTA ADCs, selectively bind to target immune cells, e.g., myeloid cells, T cells, neutrophils, monocytes, et al., the subject ADCs will be potent in many immune cells but will still alleviate or prevent adverse side effects elicited by many anti-inflammatory agents, e.g. corticosteroid receptor agonists such as dexamethasone, budesonide and other steroids, which may occur when such steroidal compounds are internalized by non-target cells.

Further, the subject ADCs, e.g., anti-VISTA ADCs which selectively bind to and internalize naive and activated target VISTA expressing immune cells, e.g., naive and activated monocytes, macrophages, T cells, T regs, CD4 T cells, CD8 T cells, neutrophils, eosinophils, dendritic cells, NK cells, and myeloid cells, may facilitate the use of reduced dosages of the inventive corticosteroid receptor agonists compared to conventional free steroids such as dexamethasone, budesonide and other steroids such as previously identified and generally known in the art. Also, the subject corticosteroid receptor agonist compounds of Formula I, II or III or corticosteroid receptor agonist-linker compounds containing when bound to antibodies which target other immune cell antigens, may be used to treat conditions wherein any or all of these specific types of immune cells which express such antigen are involved in disease pathology.

In the specific case of VISTA ADCs comprising the subject corticosteroid receptor agonist compounds of Formula I, II or III or corticosteroid receptor agonist-linker compounds containing, the subject ADCs possess a unique combination of advantages relative to previously reported ADCs for targeting and directing internalization of anti-inflammatory agents, particularly those for effecting internalization of steroids into immune cells, e.g., ADCs which target CD74, CD163, TNF, and PRLR; because of the combined benefits of VISTA as an ADC target and the specific properties of the anti-VISTA antibody which is comprised in the subject ADCs (i.e., binds to VISTA expressing immune cells at physiologic pH and possesses a very short pK but nonetheless elicits a long PD) and the advantages of the novel the subject corticosteroid receptor agonist compounds of Formula I, II or III provided herein.

Particularly, in the specific case of VISTA ADCs the subject ADCs bind to immune cells which express VISTA at very high density and notwithstanding their very short PK are efficacious (elicit anti-inflammatory activity) for prolonged duration therein, and therefore are well suited for treating chronic inflammatory or autoimmune or allergic diseases wherein prolonged and repeated administration of a steroid is therapeutically warranted.

Also, in the specific case of anti-VISTA ADCs the subject ADCs target a broad range of immune cells including neutrophils, myeloid, T cells, Tregs, macrophages, and endothelial cells; or ADCs which bind to other antigens expressed on immune cells involved in allergic, inflammatory and autoimmune responses and conditions, therefore the subject ADCs may be used to treat diseases such as inflammatory or autoimmune or allergic diseases, and conditions associated with inflammation such as heart disease, ARDS, cancer and infection involving any or all of these types of immune cells. For example, the subject ADCs may be used to treat or prevent inflammation associated with bacterial or viral infections such as COVID-19, influenza virus, pneumonia (viral or bacterial) infection and the like. However, the invention is not limited to VISTA ADCs as Applicant has shown that the novel glucocorticosteroid steroid agonist-linkers of formulae (I), (II) and (III) provided herein when linked to antibodies which target other immune antigens are also effectively internalized and release active steroid payload therein.

Further, because the subject ADCs have a rapid onset of efficacy, e.g., they can elicit anti-inflammatory activity within 2 hours of administration, they may be used for acute treatment, which may be especially beneficial in the context of treating/preventing inflammation associated with bacterial or viral infections such as COVID-19 and other coronaviruses, influenza virus, pneumonia (viral or bacterial) infection and the like which if not rapidly treated can give rise to a cytokine storm, ARDS and in worst case scenario sepsis or septic shock.

Moreover, in the specific case of anti-VISTA ADCs, VISTA, unlike some other ADC target antigens, is expressed exclusively by immune cells; therefore the subject ADCs will not be prone to internalize non-target cells.

Also, in the specific case of anti-VISTA ADCs, the subject ADCs do not bind B cells they should not be as immunosuppressive as free steroids, which should be beneficial in subjects receiving the subject ADCs repeatedly and/or for a prolonged duration since chronic steroid use has been correlated to some cancers, infections and other conditions, likely an unintended consequence of prolonged immunosuppression from prolonged steroid use. However, it should be understood that the subject ADCs when bound to antibodies which target other antigens, that these ADCs may be used to treat conditions wherein any or all of these specific types of immune cells that are bound thereby are involved in disease pathology.

Additionally, in the specific case of VISTA ADCs comprising the subject corticosteroid receptor agonist compounds of Formula I, II or III, or corticosteroid receptor agonist-linker compounds containing same, the subject ADCs act on Tregs which are an important immune cell responsible for steroid efficacy, therefore they may be more effective broadly or specifically, particularly in treating autoimmune, allergic or inflammatory conditions or inflammation involving Tregs in relation to previous ADCs comprising corticosteroid receptor agonist compounds.

Further, in the specific case of VISTA ADCs comprising the subject corticosteroid receptor agonist compounds of Formula I, II or III, or corticosteroid receptor agonist-linker compounds containing same, the subject ADCs act on both resting (naïve) and activated immune cells, e.g., monocytes, macrophages, T cells, T regs, CD4 T cells, CD8 T cells, neutrophils, eosinophils, dendritic cells, NK cells, and myeloid cells, (VISTA constitutively expressed thereon) and consequently the subject ADCs will remain active (elicit anti-inflammatory activity) both in active and remission phases of allergic, inflammatory and autoimmune conditions.

Moreover, because VISTA ADCs comprising the subject corticosteroid receptor agonist compounds of Formula I, II or III, act on neutrophils, which immune cells are critical for acute inflammation, the subject ADCS will be useful in treating acute inflammation and/or inflammatory or autoimmune or allergic conditions characterized by infrequent or sporadic inflammatory episodes.

Also, the subject ADCs which comprise a novel glucocorticosteroid steroid agonist-linker of formulae (I), (II) or (III) advantageously internalize immune cells rapidly and deliver large amounts of active steroid payload resulting in rapid and prolonged efficacy.

In the specific case of VISTA ADCs the ADCs have been shown to internalize immune cells very rapidly (e.g., within about a half hour) because VISTA cell surface turnover is high, which further indicates that the subject ADCS are well suited for treating acute inflammation and/or inflammatory or autoimmune or allergic conditions characterized by infrequent or sporadic inflammatory episodes.

Further, in some instances the subject ADCs will possess a very short half-life (PK) and only bind immune cells; therefore the subject ADCs should not less prone to target related toxicities and undesired peripheral steroid exposure (low non-specific loss effects) compared to other ADCs comprising antibodies of conventional (longer) pKs such as Humira.

Yet further in some embodiments the subject ADCs' biological activity (anti-inflammatory action) is entirely attributable to the anti-inflammatory payload (steroid) comprised therein, i.e., in instances wherein the antibody, e.g., anti-VISTA antibody possesses a silent IgG such as a silent IgG1 or IgG2 Fc region it elicits no VISTA-mediated immunological functions (no blocking of any VISTA biology).

Based at least on the foregoing combination of advantages the subject ADCs should be well suited for acute and chronic usage, and will be suitable for both therapeutic and prophylactic usage, i.e., for reducing or inhibiting inflammation, preventing the onset of inflammation, prolonging the non-active phase of the disease, and for use in treating a myriad of different types of inflammatory, allergic and autoimmune diseases.

As mentioned, in some embodiments the subject ADCs comprise an anti-VISTA antibody which binds to VISTA, (generally human VISTA) expressing immune cells at physiologic pH conditions and which possesses a short half-life or PK. Typically, these antibodies will comprise a silent Fc or no Fc and the binding of the ADC to VISTA expressing cells will not elicit any effect on VISTA signaling or VISTA-mediated effects on immunity.

By contrast, in some embodiments the antibody in the ADC, e.g., an anti-VISTA antibody or an antibody which targets another immune cell antigen will comprise a functional IgG, e.g., a functional IgG1, IgG2, IgG3 or IgG4. In the case of an anti-VISTA antibody comprising a functional IgG2, such ADC may promote VISTA or other immune cell antigen mediated-signaling or VISTA or other immune cell antigen associated functions such as suppression of T cell proliferation and T cell activity and suppression of some pro-inflammatory cytokines. This may yield additive or synergistic effects on the suppression of inflammation, allergic reactions and/or autoimmunity,

The CDRs and variable sequences of exemplary anti-VISTA antibodies and antibody fragments, i.e., which possess fragment possesses a short serum half-life under physiological pH conditions (≈pH 7.5), e.g., wherein the serum half-life of the antibody or fragment in a cynomolgus monkey or human generally is around 2.3 days $0.7 days, or less and in a rodent (human VISTA knock-in) is generally 1 to 72 hours, 1 to 32 hours, 1 to 16 hours, 1 to 8 hours, 1 to 4 hours or 1-2 hours±0.5 hour in a human VISTA knock-in rodent or =3.5, 3, 2.5, or 2.3 days±0.5 days in a primate (Cynomolgus macaque) at physiological conditions (≈pH 7.5) may be found in FIG. 8, 10 and FIG. 12.

Exemplary inflammatory agents which may be incorporated into the inventive ADCs, i.e., which may be conjugated to anti-VISTA antibodies and anti-VISTA antibody fragments, e.g., via a linker and optionally further by an heterobifunctional group include steroid or corticosteroid receptor agonists such as corticosteroids previously generically described and more specifically budesonide, beclomethasone, betamethasone, Ciclesonide, cortisol, cortisone, cortisone acetate, 16-alpha hydroxyprednisolone, dexamethasone, difluorasone, ethamethasoneb, flumethasone, flunisolide, fluocinolone acetonide, fludrocortisone, fluticasone propionate (Flovent™, Flonase™), hydrocortisone, ciclesonide, methylprednisolone, prednisone, prednisolone, mometasone, Pulmicort, triamcinolone, triamcinolone acetonide or another steroid compound or derivative thereof possessing anti-inflammatory or steroid activity and in particular include the novel steroids of Formula I, II or III and steroid-linkers according to the invention, and functional derivatives thereof. Preferred exemplary ADCs, steroids, and steroid-linkers according to the invention are disclosed in the examples infra, particularly in Example 3, in the Exemplary Embodiments section, and are depicted in FIG. 118A-O.

It is contemplated that the subject ADCs may be used to treat a subject, e.g., human or non-human mammal having any condition wherein alleviation of inflammation is therapeutically warranted by use of an anti-inflammatory agent such as a steroid. Such conditions may be associated with acute or chronic inflammation, e.g., sporadic or episodic. In some preferred embodiments the subject will have a condition that requires repeated and/or high dosages of the anti-inflammatory agent such as a corticosteroid receptor agonist wherein dosing under conventional conditions, i.e., wherein the anti-inflammatory is naked or unconjugated, the drug may elicit undesired side effects such as toxicity to non-targeted cells. Such conditions include autoimmune and inflammatory conditions. Non-limiting examples of such conditions include of allergy, autoimmunity, transplant, gene therapy, inflammation, GVHD or sepsis, infection, cancer or to treat or prevent inflammatory, autoimmune, or allergic side effects associated with any of the foregoing conditions in a human subject.

In some other preferred embodiments the subject will have an acute or chronic inflammatory condition or flare-up wherein a rapid onset of efficacy is therapeutically desirable, e.g., an inflammatory condition characterized by repeated acute inflammatory episodes, frequent or infrequent, optionally wherein repeated and/or high dosages of the anti-inflammatory agent such as a corticosteroid receptor agonist is therapeutically warranted, and optionally wherein dosing under conventional conditions, i.e., wherein the anti-inflammatory is naked or unconjugated, the drug may elicit undesired side effects such as toxicity to non-targeted cells. Such conditions include autoimmune and inflammatory conditions, cancer, and infectious conditions associated with inflammation, e.g., characterized by acute and/or severe inflammatory episodes.

Non-limiting examples of such conditions include allergy, autoimmunity, transplant, gene therapy, inflammation, cancer, GVHD or sepsis, infection (e.g., bacterial, viral, fungal, parasitic), acute respiratory distress syndrome (ARDS) or to treat or prevent inflammatory, autoimmune, or allergic side effects associated with any of the foregoing conditions in a human subject.

Other specific exemplary conditions wherein use of the subject ADCs may be beneficial include, rheumatoid arthritis, juvenile idiopathic arthritis, psoriatic arthritis, ankylosing spondylitis, adult Crohn's disease, pediatric Crohn's disease, ulcerative colitis, plaque psoriasis, hidradenitis suppurativa, uveitis, Bechet's disease, a spondyloarthropathy, or psoriasis.

Other exemplary conditions and instances wherein use of the subject ADCs may be therapeutically beneficial include:
(i) conditions primarily only effectively treatable with high doses of steroids, optionally polymyalgia rheumatica and/or giant cell arteritis, which patient optionally has been treated or is undergoing treatment with high steroid doses;
(ii) conditions with a comorbidity limiting steroid use, optionally diabetes mellitis, nonalcoholic steatohepatitis (NASH), morbid obesity avascular necrosis/osteonecrosis (AVN), glaucoma. Steroid-induced hypertension, severe skin fragility, and/or osteoarthritis;
(iii) conditions wherein safe long-term treatment agents are available, but wherein several months of induction with high-doses of steroids is desired, optionally AAV, polymyositis, dermamyositis, lupus, inflammatory lung disease, autoimmune hepatitis, inflammatory bowel disease, immune thrombocytopenia, autoimmune hemolytic anemia, gout patients wherein several months of induction with high-doses of steroids is therapeutically warranted;
(iv) dermatologic conditions that require short/long-term treatment, optionally of uncertain treatment or duration and/or no effective alternative to steroid administration, optionally Stevens Johnson, other severe drug eruption conditions, conditions involving extensive contact dermatitis, other severe immune-related dermatological conditions such as PG, LCV, Erythroderma and the like;
(v) conditions treated with high-dose corticosteroids for flares/reoccurrences, optionally COPD, asthma, lupus, gout, pseudogout;
(vi) immune-related neurologic diseases such as small-fiber neuropathy, MS (subset), chronic inflammatory demyelinating polyneuropathy, myasthenia gravis and the like;
(vii) hematological/oncology indications, optionally wherein high doses of steroids would potentially be therapeutically warranted or beneficial;
(viii) ophthalmologic conditions, optionally uveitis, iritis, scleritis, and the like;
(ix) conditions associated with permanent or very prolonged adrenal insufficiency or secondary adrenal insufficiency, optionally latrogenic Addisonian crisis;
(x) conditions often treated with long term, low dose steroids, optionally lupus, RA, psA, vasculitis, and the like; and
(xi) special classes of patients such as pregnant/breast-feeding women, pediatric patients optionally those with growth impairment or cataracts.

Compositions containing ADCs or novel glucocorticosteroids of Formula I, II or II according to the invention or steroid-linkers containing same may be used alone or in association with other therapeutics, especially other immunosuppressant molecules or anti-inflammatories or other therapeutics used in treating autoimmune, allergic and inflammatory conditions such as drugs used in the treatment of e.g., acquired immune deficiency syndrome (AIDS), acquired splenic atrophy, acute anterior uveitis, Acute Disseminated Encephalomyelitis (ADEM), acute gouty arthritis, acute necrotizing hemorrhagic leukoencephalitis, acute or chronic sinusitis, acute purulent meningitis (or other central nervous system inflammatory disorders), acute serious inflammation, Addison's disease, adrenalitis, adult onset diabetes mellitus (Type II diabetes), adult-onset idiopathic hypoparathyroidism (AOIH), Agammaglobulinemia, agranulocytosis, vasculitides, including vasculitis, optionally, large vessel vasculitis, optionally, polymyalgia rheumatica and giant cell (Takayasu's) arthritis, allergic conditions, allergic contact dermatitis, allergic dermatitis, allergic granulomatous angiitis, allergic hypersensitivity disorders, allergic neuritis, allergic reaction, alopecia areata, alopecia totalis, Alport's syndrome, alveolitis, optionally allergic alveolitis or fibrosing alveolitis, Alzheimer's disease, amyloidosis, amylotrophic lateral sclerosis (ALS; Lou Gehrig's disease), an eosinophil-related disorder, optionally eosinophilia, anaphylaxis, ankylosing spondylitis, angiectasis, antibody-mediated nephritis, Anti-GBM/Anti-TBM nephritis, antigen-antibody complex-mediated diseases, antiglomerular basement membrane disease, anti-phospholipid antibody syndrome, antiphospholipid syndrome (APS), aphthae, aphthous stomatitis, aplastic anemia, arrhythmia, arteriosclerosis, arteriosclerotic disorders, arthritis, optionally rheumatoid arthritis such as acute arthritis, or chronic rheumatoid arthritis, arthritis chronica progrediente, arthritis deformans, ascariasis, aspergilloma, granulomas containing eosinophils, aspergillosis, aspermiogenese, asthma, optionally asthma bronchiale, bronchial asthma, or auto-immune asthma, ataxia telangiectasia, ataxic sclerosis, atherosclerosis, autism, autoimmune angioedema, autoimmune aplastic anemia, autoimmune atrophic gastritis, autoimmune diabetes, autoimmune disease of the testis and ovary including autoimmune orchitis and oophoritis, autoimmune disorders associated with collagen disease, autoimmune dysautonomia, autoimmune ear disease, optionally autoimmune inner ear disease (AGED), autoimmune endocrine diseases including thyroiditis such as autoimmune thyroiditis, autoimmune enteropathy syndrome, autoimmune gonadal failure, autoimmune hearing loss, autoimmune hemolysis, Autoimmune hepatitis, autoimmune hepatological disorder, autoimmune hyperlipidemia, autoimmune immunodeficiency, autoimmune inner ear disease (AIED), autoimmune myocarditis, autoimmune neutropenia, autoimmune pancreatitis, autoimmune polyendocrinopathies, autoimmune polyglandular syndrome type I, autoimmune retinopathy, autoimmune thrombocytopenia purpura (ATP), autoimmune thyroid disease, autoimmune urticaria, autoimmune-mediated gastrointestinal diseases, Axonal & neuronal neuropathies, Balo disease, Behçet's disease, benign familial and ischemia-reperfusion injury, benign lymphocytic angiitis, Berger's disease (IgA nephropathy), bird-fancier's lung, blindness, Boeck's disease, bronchiolitis obliterans (non-transplant) VS NSIP, bronchitis, bronchopneumonic aspergillosis, Bruton's syndrome, bullous pemphigoid, Caplan's syndrome, Cardiomyopathy, cardiovascular ischemia, Castleman's syndrome, Celiac disease, celiac sprue (gluten enteropathy), cerebellar degeneration, cerebral ischemia, and disease accompanying vascularization, Chagas disease, channelopathies, optionally epilepsy, channelopathies of the CNS, chorioretinitis, choroiditis, an autoimmune hematological disorder, chronic active hepatitis or autoimmune chronic active hepatitis, chronic contact dermatitis, chronic eosinophilic pneumonia, chronic fatigue syndrome, chronic hepatitis, chronic hypersensitivity pneumonitis, chronic inflammatory arthritis, Chronic inflammatory demyelinating polyneuropathy (CIDP), chronic intractable inflammation, chronic mucocutaneous candidiasis, chronic neuropathy, optionally IgM polyneuropathies or IgM-mediated neuropathy, chronic obstructive airway disease, chronic pulmonary inflammatory disease, Chronic recurrent multifocal osteomyelitis (CRMO), chronic thyroiditis (Hashimoto's thyroiditis) or subacute thyroiditis, Churg-Strauss syndrome, cicatricial pemphigoid/benign mucosal pemphigoid, coronavirus mediated infections such as SARS-CoV-2 (COVID-19), SARS-CoV, MERS, SARS-CoV-2 and associated side-effects, CNS inflammatory disorders, CNS vasculitis, Coeliac disease, Cogan's syndrome, cold agglutinin disease, colitis polyposa, colitis such as ulcerative colitis, colitis ulcerosa, collagenous colitis, conditions involving infiltration of T cells and chronic inflammatory responses, congenital heart block, congenital rubella infection, Coombs positive anemia, coronary artery disease, Coxsackie myocarditis, CREST syndrome (calcinosis, Raynaud's phenomenon), Crohn's disease, cryoglobulinemia, Cushing's syndrome, cyclitis, optionally chronic cyclitis, heterochronic cyclitis, iridocyclitis, or Fuch's cyclitis, cystic fibrosis, cytokine-induced toxicity, deafness, degenerative arthritis, demyelinating diseases, optionally autoimmune demyelinating diseases, demyelinating neuropathies, dengue, dermatitis herpetiformis and atopic dermatitis, dermatitis including contact dermatitis, dermatomyositis, dermatoses with acute inflammatory components, Devic's disease (neuromyelitis optica), diabetic large-artery disorder, diabetic nephropathy, diabetic retinopathy, Diamond Blackfan anemia, diffuse interstitial pulmonary fibrosis, dilated cardiomyopathy, discoid lupus, diseases involving leukocyte diapedesis, Dressler's syndrome, Dupuytren's contracture, echovirus infection, eczema including allergic or atopic eczema, encephalitis such as Rasmussen's encephalitis and limbic and/or brainstem encephalitis, encephalomyelitis, optionally allergic encephalomyelitis or encephalomyelitis allergica and experimental allergic encephalomyelitis (EAE), endarterial hyperplasia, endocarditis, endocrine ophthalmopathy, endometriosis, endomyocardial fibrosis, endophthalmia phacoanaphylactica, endophthalmitis, enteritis allergica, eosinophilia-myalgia syndrome, eosinophilic fascitis, epidemic keratoconjunctivitis, epidermolysis bullosa acquisita (EBA), episclera, episcleritis, Epstein-Barr virus infection, erythema elevatum et diutinum, erythema multiforme, erythema nodosum leprosum, erythema nodosum, erythroblastosis fetalis, esophageal dysmotility, Essential mixed cryoglobulinemia, ethmoid, Evan's syndrome, Experimental Allergic Encephalomyelitis (EAE), Factor VIII deficiency, farmer's lung, febris rheumatica, Felty's syndrome, fibromyalgia, fibrosing alveolitis, filariasis, focal segmental glomerulosclerosis (FSGS), food poisoning, frontal, gastric atrophy, giant cell arthritis (temporal arthritis), giant cell hepatitis, giant cell polymyalgia, glomerulonephritides, glomerulonephritis (GN) with and without nephrotic syndrome such as chronic or acute glomerulonephritis (e.g., primary GN), Goodpasture's syndrome, gouty arthritis, granulocyte transfusion-associated syndromes, granulomatosis including lymphomatoid granulomatosis, granulomatosis with polyangiitis (GPA), granulomatous uveitis, Grave's disease, Guillain-Barre syndrome, gutatte psoriasis, hemoglobinuria paroxysmatica, Hamman-Rich's disease, Hashimoto's disease, Hashimoto's encephalitis, Hashimoto's thyroiditis, hemochromatosis, hemolytic anemia or immune hemolytic anemia including autoimmune hemolytic anemia (AIHA), hemolytic anemia, hemophilia A, Henoch-Schonlein purpura, Herpes gestationis, human immunodeficiency virus (HIV) infection, hyperalgesia, hypogammaglobulinemia, hypogonadism, hypoparathyroidism, idiopathic diabetes insipidus, idiopathic facial paralysis, idiopathic hypothyroidism, idiopathic IgA nephropathy, idiopathic membranous GN or idiopathic membranous nephropathy, idiopathic nephritic syndrome, idiopathic pulmonary fibrosis, idiopathic sprue, Idiopathic thrombocytopenia purpura (ITP), IgA nephropathy, IgE-mediated diseases, optionally anaphylaxis and allergic or atopic rhinitis, IgG4-related sclerosing disease, ileitis regionalis, immune complex nephritis, immune responses associated with acute and delayed hypersensitivity mediated by cytokines and T-lymphocytes, immune-mediated GN, immunoregulatory lipoproteins, including adult or acute respiratory distress syndrome (ARDS), Inclusion body myositis, infectious arthritis, infertility due to antispermatozoan antibodies, inflammation of all or part of the uvea, inflammatory bowel disease (IBD) inflammatory hyperproliferative skin diseases, inflammatory myopathy, insulin-dependent diabetes (type 1), insulitis, Interstitial cystitis, interstitial lung disease, interstitial lung fibrosis, iritis, ischemic re-perfusion disorder, joint inflammation, Juvenile arthritis, juvenile dermatomyositis, juvenile diabetes, juvenile onset (Type I) diabetes mellitus, including pediatric insulin-dependent diabetes mellitus (IDDM), juvenile-onset rheumatoid arthritis, Kawasaki syndrome, keratoconjunctivitis sicca, kypanosomiasis, Lambert-Eaton syndrome, leishmaniasis, leprosy, leucopenia, leukocyte adhesion deficiency, Leukocytoclastic vasculitis, leukopenia, lichen planus, lichen sclerosus, ligneous conjunctivitis, linear IgA dermatosis, Linear IgA disease (LAD), Loffler's syndrome, lupoid hepatitis, lupus (including nephritis, cerebritis, pediatric, non-renal, extra-renal, discoid, alopecia), Lupus (SLE), lupus erythematosus disseminatus, Lyme arthritis, Lyme disease, lymphoid interstitial pneumonitis, malaria, male and female autoimmune infertility, maxillary, medium vessel vasculitis (including Kawasaki's disease and polyarteritis nodosa), membrano- or membranous proliferative GN (MPGN), including Type I and Type II, and rapidly progressive GN, membranous GN (membranous nephropathy), Meniere's disease, meningitis, microscopic colitis, microscopic polyangiitis, migraine, minimal change nephropathy, Mixed connective tissue disease (MCTD), mononucleosis infectiosa, Mooren's ulcer, Mucha-Habermann disease, multifocal motor neuropathy, multiple endocrine failure, multiple organ injury syndrome such as those secondary to septicemia, trauma or hemorrhage, multiple organ injury syndrome, multiple sclerosis (MS) such as spino-optical MS, multiple sclerosis, mumps, muscular disorders, myasthenia gravis such as thymoma-associated myasthenia gravis, myasthenia gravis, myocarditis, myositis, narcolepsy, necrotizing enterocolitis, and transmural colitis, and autoimmune inflammatory bowel disease, necrotizing, cutaneous, or hypersensitivity vasculitis, neonatal lupus syndrome (NLE), nephrosis, nephrotic syndrome, neurological disease, neuromyelitis optica (Devic's), neuromyelitis optica, neuromyotonia, neutropenia, non-cancerous lymphocytosis, nongranulomatous uveitis, non-malignant thymoma, ocular and orbital inflammatory disorders, ocular cicatricial pemphigoid, oophoritis, ophthalmia symphatica, opsoclonus myoclonus syndrome (OMS), opsoclonus or opsoclonus myoclonus syndrome (OMS), and sensory neuropathy, optic neuritis, orchitis granulomatosa, osteoarthritis, palindromic rheumatism, pancreatitis, pancytopenia, PANDAS (Pediatric Autoimmune Neuropsychiatric Disorders Associated with Streptococcus), paraneoplastic cerebellar degeneration, paraneoplastic syndrome, paraneoplastic syndromes, including neurologic paraneoplastic syndromes, optionally Lambert-Eaton myasthenic syndrome or Eaton-Lambert syndrome, parasitic diseases such as Leishmania, paroxysmal nocturnal hemoglobinuria (PNH), Parry Romberg syndrome, pars planitis (peripheral uveitis), Parsonnage-Turner syndrome, parvovirus infection, pemphigoid such as pemphigoid bullous and skin pemphigoid, pemphigus (including pemphigus vulgaris), pemphigus erythematosus, pemphigus foliaceus, pemphigus mucus-membrane pemphigoid, pemphigus, peptic ulcer, periodic paralysis, peripheral neuropathy, perivenous encephalomyelitis, pernicious anemia (anemia perniciosa), pernicious anemia, phacoantigenic uveitis, pneumonocirrhosis, POEMS syndrome, polyarteritis nodosa, Type I, II, & III, polyarthritis chronica primaria, polychondritis (e.g., refractory or relapsed polychondritis), polyendocrine autoimmune disease, polyendocrine failure, polyglandular syndromes, optionally autoimmune polyglandular syndromes (or polyglandular endocrinopathy syndromes), polymyalgia rheumatica, polymyositis, polymyositis/dermatomyositis, polyneuropathies, polyradiculitis acuta, post-cardiotomy syndrome, posterior uveitis, or autoimmune uveitis, postmyocardial infarction syndrome, postpericardiotomy syndrome, post-streptococcal nephritis, post-vaccination syndromes, presenile dementia, primary biliary cirrhosis, primary hypothyroidism, primary idiopathic myxedema, primary lymphocytosis, which includes monoclonal B cell lymphocytosis, optionally benign monoclonal gammopathy and monoclonal garnmopathy of undetermined significance, MGUS, primary myxedema, primary progressive MS (PPMS), and relapsing remitting MS (RRMS), primary sclerosing cholangitis, progesterone dermatitis, progressive systemic sclerosis, proliferative arthritis, psoriasis such as plaque psoriasis, psoriasis, psoriatic arthritis, pulmonary alveolar proteinosis, pulmonary infiltration eosinophilia, pure red cell anemia or aplasia (PRCA), pure red cell aplasia, purulent or nonpurulent sinusitis, pustular psoriasis and psoriasis of the nails, pyelitis, pyoderma gangrenosum, Quervain's thyroiditis, Raynaud's phenomenon, reactive arthritis, recurrent abortion, reduction in blood pressure response, reflex sympathetic dystrophy, refractory sprue, Reiter's disease or syndrome, relapsing polychondritis, reperfusion injury of myocardial or other tissues, reperfusion injury, respiratory distress syndrome, restless legs syndrome, retinal autoimmunity, retroperitoneal fibrosis, Reynaud's syndrome, rheumatic diseases, rheumatic fever, rheumatism, rheumatoid arthritis, rheumatoid spondylitis, rubella virus infection, Sampter's syndrome, sarcoidosis, schistosomiasis, Schmidt syndrome, SCID and Epstein-Barr virus-associated diseases, sclera, scleritis, sclerodactyl, scleroderma, optionally systemic scleroderma, sclerosing cholangitis, sclerosis disseminata, sclerosis such as systemic sclerosis, sensoneural hearing loss, seronegative spondyloarthritides, Sheehan's syndrome, Shulman's syndrome, silicosis, Sjögren's syndrome, sperm & testicular autoimmunity, sphenoid sinusitis, Stevens-Johnson syndrome, stiff-man (or stiff-person) syndrome, subacute bacterial endocarditis (SBE), subacute cutaneous lupus erythematosus, sudden hearing loss, Susac's syndrome, Sydenham's chorea, sympathetic ophthalmia, systemic lupus erythematosus (SLE) or systemic lupus erythematodes, cutaneous SLE, systemic necrotizing vasculitis, ANCA-associated vasculitis, optionally Churg-Strauss vasculitis or syndrome (CSS), tabes dorsalis, Takayasu's arteritis, telangiectasia, temporal arteritis/Giant cell arteritis, thromboangiitis ubiterans, thrombocytopenia, including thrombotic thrombocytopeniarpura (TTP) and autoimmune or immune-mediated thrombocytopenia such as idiopathic thrombocytopenia purpura (ITP) including chronic or acute ITP, thrombocytopenia purpura (TTP), thyrotoxicosis, tissue injury, Tolosa-Hunt syndrome, toxic epidermal necrolysis, toxic-shock syndrome, transfusion reaction, transient hypogammaglobulinemia of infancy, transverse myelitis, traverse myelitis, tropical pulmonary eosinophilia, tuberculosis, ulcerative colitis, undifferentiated connective tissue disease (UCTD), urticaria, optionally chronic allergic urticaria and chronic idiopathic urticaria, including chronic autoimmune urticaria, uveitis, anterior uveitis, uveoretinitis, valvulitis, vascular dysfunction, vasculitis, vertebral arthritis, vesiculobullous dermatosis, vitiligo, Wegener's granulomatosis (Granulomatosis with Polyangiitis (GPA)), Wiskott-Aldrich syndrome, or x-linked hyper IgM syndrome.

The subject ADCs and novel corticosteroids of Formula I, II or III and corticosteroid-linkers containing may be used for both the prophylactic and/or therapeutic treatment of inflammation and diseases associated with inflammation including by way of example autoimmune disorders, inflammatory disorders, infectious diseases and cancer. A preferred application of the subject ADCs is for the treatment of chronic diseases associated with inflammation. Moreover, in the specific case of VISTA antibody comprising ADCs, as shown in the examples, quite unexpectedly these ADCs, notwithstanding the short pK of the anti-VISTA antibody which is comprised therein (which binds to VISTA expressing cells at physiological conditions and which is not engineered to alter or optimize pH binding or to enhance half-life), i.e., typically a pK of around 2.3 days or less in cyno and typically only a few hours in human VISTA engineered mice, has been found to maintain potency for a much more prolonged period (PD) relative to the much shorter half-life (pK) of the antibody.

As is shown herein VISTA ADC conjugates according to the invention when evaluated in vitro and in vivo models have been demonstrated to provide for PD/PK ratios of at least at least 14:1 and even 28:1 or longer in immune cells. (In fact the PD/PK ratios may be higher because the rodents or non-human primates were euthanized at the time PD was determined therefore not permitting a longer assessment of potency).

While Applicant does not want to be bound by their belief, it is theorized that VISTA ADCs comprising the subject corticosteroid receptor agonist compounds of Formula I, II or III, are delivered in very high amounts in target VISTA expressing cells such as macrophages, T cells, and Tregs and other VISTA expressing immune cells including immune cells which have long cell turnovers (weeks, months or longer). Essentially, it appears that a depot effect is created within specific types of immune cells, i.e., a large quantity or “depot” of the subject ADCs are internalized into such VISTA expressing immune cells, e.g., macrophages and myeloid cells, because of their very high surface expression of VISTA. This in turn apparently results in this depot comprising the internalized ADCs being slowly metabolized or cleaved within the immune cell, e.g., by cell enzymes. In vivo studies disclosed herein indicate that the metabolism or cleavage of internalized ADCs according to the invention apparently may occur for over a week, 2 weeks, 4 weeks or longer in a rodent or primate thereby providing for gradual and prolonged release of therapeutically effective amounts of the steroid payload within the host's immune cells. This occurs notwithstanding the fact that by that time (because of the short pk of the ADC and antibody therein) that no appreciable amount of the ADC should remain in the serum (i.e., based on the short pK of the ADC not enough of ADC molecules will still remain in the peripheral circulation in order to elicit a therapeutically significant effect on immunity).

Moreover, while these observations are highly surprising; it is anticipated since drug metabolism generally occurs much faster in rodents than in primates (i.e., drug metabolism is much slower in humans than in rodents); and further since the levels of VISTA expression and immune cells which express VISTA are similar in rodents and in humans and non-human primates, that the subject ADCs will possess similar or even greater PD/PK ratios in humans and other primates. Accordingly, the subject ADCs should be well suited for therapeutic applications wherein prolonged drug efficacy is desired or necessary.

Moreover, Applicant has found that ADCs according to the invention possess intrinsic advantages compared to previous reported ADCs because of the properties afforded by the novel glucocorticosteroids and steroid agonist-linkers of formulae (I), (II) and (III), i.e., ADCS containing same do not seem to be prone to aggregation, and provide for high DAR's and moreover very effectively internalize immune cells and deliver high amounts of active payload therein resulting in rapid and prolonged efficacy. Notwithstanding the foregoing, ADCs according to the invention which possess low DARs, e.g., a DAR of 4.0 or less have also been shown herein to exhibit good potency and may alternatively be used in therapy, e.g., because they may possess better developability properties.

As mentioned, another preferred usage of the subject ADCs and the novel glucocorticosteroids of Formula I, II or III is for acute usage, i.e., for treating acute inflammation. As is shown in the examples glucocorticosteroids of Formula I, II or III and ADCs containing according to the invention have a rapid onset of efficacy, e.g., they can elicit anti-inflammatory effects as rapid as within 2 hours after administration. Acute usage of the subject anti-VISTA ADCs is further advantageous because the subject anti-VISTA ADCs have been demonstrated to effectively target and internalize neutrophils wherein they elicit anti-inflammatory effects but not non-target cells. This is especially beneficial in acute usage as neutrophils are involved in the early stages of inflammatory responses, accordingly the subject glucocorticosteroids of Formula I, II or III and ADCs containing are also well suited for treating acute inflammatory indications.

Another preferred usage of the subject ADCs and the novel corticosteroids of Formula I, II or III is for maintenance therapy. In the specific case of VISTA ADCs, because VISTA is expressed on both activated and non-activated (naïve) immune cells, e.g., monocytes, macrophages, T cells, T regs, CD4 T cells, CD8 T cells, neutrophils, eosinophils, dendritic cells, NK cells, and myeloid cells, (VISTA is constitutively expressed thereby), the subject ADCs can be administered periodically, over a prolonged time period, and such administration will elicit anti-inflammatory activity both when the treated subject is in the active stage of an inflammatory response as well as when the subject is in disease remission. This is therapeutically beneficial as many chronic autoimmune. allergic and inflammatory disorders are known to be characterized by active periods or episodes wherein the patient experiences inflammation and other symptoms or pathologic reactions associated with the disease and periods of remission wherein the disease symptoms including inflammation and other symptoms or pathologic reactions associated with the disease are not present or are much less severe (i.e., remitting/relapsing or episodic). It is anticipated that because the subject ADCs bind to both activated and non-activated immune cells, e.g., e.g., monocytes, macrophages, T cells, T regs, CD4 T cells, CD8 T cells, neutrophils, eosinophils, dendritic cells, NK cells, and myeloid cells, that a patient treated with the subject ADCs may more effectively maintain disease remission, i.e., the period of remission should be more prolonged and/or the active phase of the disease may manifest in a much less severe form because of the maintained anti-inflammatory efficacy of the subject ADCs on target immune cells both during active disease and during remission.

Moreover, the subject ADCs should be well suited for prolonged or chronic usage because of their absence of any effect on non-target cells, i.e. non-immune cells. As is shown in the examples infra the subject ADCs virtually exclusively act on target immune cells and not on non-immune cells (some anti-inflammatory activity was detected in the liver, however, this is likely explained by the fact that the liver comprises immune cells).

Also, in embodiments wherein the pK of the subject ADCs is short (but surprisingly still have a long PD) the subject ADCs do not remain the serum for prolonged duration, i.e., they rapidly bind to and are internalized by immune cells wherein they deliver their anti-inflammatory payload and are potent for prolonged duration, apparently because the ADCs are efficiently and rapidly taken up in large amounts by immune cells and are slowly metabolized within these immune cells. Therefore, since the subject ADCs are only present in the peripheral circulation for short duration, the subject ADCs have limited opportunity to interact with non-target cells as compared to ADCs which have a long pK because the antibody comprised therein possess a long PK (which is conventional for most therapeutic antibodies). A particular example is ADCs which comprise Humira, which like most conventional therapeutic antibodies possesses a long pK i.e., around a month.

Still further the subject ADCs should be well suited for prolonged or chronic usage because in some embodiments the efficacy of ADCs according to the invention (particularly anti-VISTA ADCs according to the invention comprising Fc regions engineered to impair FcR and complement binding) is entirely attributable to the anti-inflammatory payload, e.g., a steroid of formula (I), (II) or (III). Essentially the antibody, e.g., an anti-VISTA antibody in such instance only provides a targeting function, i.e., it facilitates the binding and internalization of the ADC by target immune cells. However, the binding of such ADC to a VISTA expressing immune cell does not modulate the activity of VISTA, i.e., the anti-VISTA antibody comprising an Fc engineered to preclude Fc crosslinking does not antagonize or agonize VISTA activity.[This is to be contrasted to existing ADCs for delivery of steroids comprising an antibody which elicits a biological effect upon binding to target antigen (such as Humira ADCs)]. This should be beneficial from a dosing perspective as ADC potency only depends on the anti-inflammatory payload. Also, this is further therapeutically beneficial as anti-VISTA or other immune cell antigen targeting antibodies could otherwise in some instances elicit a proinflammatory cytokine response which could be undesirable in the context of a drug the objective of which is to alleviate inflammation.

Acute and chronic autoimmune and inflammatory indications wherein the subject ADCs may be used have been afore-mentioned and include Acquired aplastic anemia+, Acquired hemophilia+, Acute disseminated encephalomyelitis (ADEM)+, Acute hemorrhagic leukoencephalitis (AHLE)/Hurst's disease+, Agammaglobulinemia, primary+, Alopecia areata+, Ankylosing spondylitis (AS), Anti-NMDA receptor encephalitis+, Antiphospholipid syndrome (APS)+, Arteriosclerosis, Autism spectrum disorders (ASD), Autoimmune Addison's disease (AAD)+, Autoimmune dysautonomia/Autoimmune autonomic ganglionopathy (AAG), Autoimmune encephalitis+, Autoimmune gastritis, Autoimmune hemolytic anemia (AIHA)+, Autoimmune hepatitis (AIH)+, Autoimmune hyperlipidemia, Autoimmune hypophysitis/lymphocytic hypophysitis+, Autoimmune inner ear disease (AIED)+, Autoimmune lymphoproliferative syndrome (ALPS)+, Autoimmune myocarditis, Autoimmune oophoritis+, Autoimmune orchitis+, Autoimmune pancreatitis (AIP)/Immunoglobulin G4-Related Disease (IgG4-RD)+, Autoimmune polyglandular syndromes, Types I, II, & III+, Autoimmune progesterone dermatitis+, Autoimmune sudden sensorineural hearing loss (SNHL) Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Diabetes, type 1, Dermatitis herpetiformis, Dermatomyositis, Devic's disease (neuromyelitis optica). Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Fibrosing alveolitis, Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopeniarpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus (including nephritis and cutaneous), Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myelin Oligodendrocyte Glycoprotein Antibody Disorder, Myositis, Narcolepsy, Neonatal Lupus, Neuromyelitis optica, Neutropenia, Ocular cicatricial pemphigoid, Optic neuritis, Opsoclonus-myoclonus syndrome (OMS), Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary Biliary Cholangitis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopeniaurpura (TTP), Thyroid eye disease (TED), Tolosa-Hunt syndrome (THS), Transverse myelitis, Type 1 diabetes, Undifferentiated connective tissue disease (UCTD), Uveitis, Vasculitis, Vitiligo, Vogt-Koyanagi-Harada Disease, among others.

Preferred indications wherein the ADCs should be therapeutically effective include Severe asthma, COPD, Giant cell arteritis, ANKA vasculitis and IBD (Colitis (e.g., ulcerative) and Crohns). Of course, it should be understood that the disease conditions identified herein are intended to be exemplary and not exhaustive.

The subject ADCs may be combined with other therapeutics which may be administered in the same or different compositions, at the same or different time. For example, the subject ADCs may be administered in a therapeutic regimen that includes the administration of a PD-1 or PD-L1 agonist, CTLA4-Ig, a cytokine, a cytokine agonist or antagonist, or another immunosuppressive receptor agonist or antagonist.

Other examples of specific immmunoinhibitory molecules that may be combined with ADCs according to the invention include antibodies that block a costimulatory signal (e.g., against CD28 or ICOS), antibodies that activate an inhibitory signal via CTLA4, and/or antibodies against other immune cell markers (e.g., against CD40, CD40 ligand, or cytokines), fusion proteins (e.g., CTLA4-Fc or PD-1-Fc), and immunosuppressive drugs (e.g., rapamycin, cyclosporine A, or FK506).

### Modified Fc Region in ADCs According to the Invention

As mentioned, in some preferred embodiments of the invention the ADC comprises an Fc which may be engineered to include modifications within the Fc region, typically to alter one or more functional properties of the antibody, such as complement fixation, Fc receptor binding, and/or antigen-dependent cellular cytotoxicity. Furthermore, in some embodiments of the invention the ADC may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CHI is altered to, for example, facilitate assembly of the light and heavy chains or to increase or decrease the stability of the antibody.

In another embodiment, the Fc hinge region of an antibody is mutated to further decrease the biological half-life of the antibody. More specifically, one or more amino acid mutations are introduced into the CH2-CH3 domain interface region of the Fc-hinge fragment such that the antibody has impaired Staphylococcal protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector functions of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the CI component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region in the ADC is modified to increase the affinity of the antibody for an Fγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (See Shields, R. L. et al. (2001) J. Biol. Chem. 276:6591-6604). Specific mutations at positions 256, 290, 298, 333, 334 and 339 are shown to improve binding to FcγRIII. Additionally, the following combination mutants are shown to improve FcγRIII binding: T256A/S298A, S298A/E333A, S298A/K224A and S298A/E333A/K334A. Furthermore, mutations such as M252Y/S254T/T256E or M428L/N434S improve binding to FcRn and increase antibody circulation half-life (See Chan C A and Carter P J (2010) Nature Rev Immunol 10:301-316).

In still another embodiment, the antibody in the ADC can be modified to abrogate in vivo Fab arm exchange. Specifically, this process involves the exchange of IgG4 half-molecules (one heavy chain plus one light chain) between other IgG4 antibodies that effectively results in b specific antibodies which are functionally monovalent. Mutations to the hinge region and constant domains of the heavy chain can abrogate this exchange (See Aalberse, R C, Schuurman J., 2002, Immunology 105:9-19).

In still another embodiment, the glycosylation of an antibody in the ADC s modified. For example, an aglycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally, or alternatively, an antibody in the ADC can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. For example, the cell lines Ms704, Ms705, and Ms709 lack the fucosyltransferase gene, FUT8 (a (1,6) fucosyltransferase), such that antibodies expressed in the Ms704, Ms705, and Ms709 cell lines lack fucose on their carbohydrates. The Ms704, Ms705, and Ms709 FUT8 cell lines are created by the targeted disruption of the FUT8 gene in CHO/DG44 cells using two replacement vectors (See U.S. Patent Publication No. 20040110704 by Yamane et al. and Yamane-Ohnuki et al. (2004) Biotechnol Bioeng 87:614-22). As another example, EP 1, 176, 195 by Hanai et al. describes a cell line with a functionally disrupted FUT8 gene, which encodes a fucosyl transferase, such that antibodies expressed in such a cell line exhibit hypofucosylation by reducing or eliminating the a 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (See also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., P(I,4)-N-acetylglucosaminyltransferase III (GnTIII)) such that antibodies expressed in the engineered cell lines exhibit increased bisecting GlcNac structures which results in increased ADCC activity of the antibodies (See also Umana et al. (1999) Nat. Biotech. 17:176-180). Alternatively, the fucose residues of the antibody may be cleaved off using a fucosidase enzyme. For example, the fucosidase α-L-fucosidase removes fucosyl residues from antibodies (Tarentino, A. L. et al. (1975) Biochem. 14:5516-23).

As mentioned in the exemplary embodiments the Fc region of the antibody is mutated to impair FcR binding and optionally to impair complement binding. These mutations include those mutations comprised in their exemplary antibodies. These mutations include any or all of L234A/L235A and L234A/L235A/E269R/K322A (IgG1 Fc); and V234A/G237A/P238s.V309L/A330S/P331S (IgG2 Fc).

### Ex Vivo Use of ADCs According to the Invention

According to at least some embodiments, immune cells, e.g., monocytes or myeloid cells, neutrophils, monocytes, T cells, B cells, NK cells, macrophages, mast cells, dendritic cells, Tregs, and other hematopoietic cells, among other immune cell types can be contacted ex vivo with the subject ADCs to elicit anti-inflammatory responses and the contacted cells then infused into a patient, e.g., one having an allergic, autoimmune or inflammatory condition wherein reduction of inflammation is therapeutically desired. modulate immune responses.

Exemplary Uses of Subject ADCs and Pharmaceutical Compositions Containing for Treatment of Autoimmune Disease

The ADCs and novel steroids of Formula I, II or III described herein may be used for treating an immune system related disease. Optionally, the immune system related condition comprises an autoimmune or inflammatory disease such as those identified previously, e.g., transplant rejection, severe asthma, colitis or IBD, graft-versus-host disease. Optionally the treatment is combined with another moiety useful for treating immune related condition.

Thus, treatment of multiple sclerosis using the subject ADCs may be combined with, for example, any known therapeutic agent or method for treating multiple sclerosis, optionally as described herein.

Thus, treatment of rheumatoid arthritis or other arthritic condition, using the subject ADCs may be combined with, for example, any known therapeutic agent or method for treating rheumatoid arthritis, optionally as described herein.

Thus, treatment of IBD, using the using the subject ADCs may be combined with, for example, any known therapeutic agent or method for treating IBD, optionally as described herein.

Thus, treatment of psoriasis, using the subject ADCs may be combined with, for example, any known therapeutic agent or method for treating psoriasis, optionally as described herein.

Thus, treatment of type 1 diabetes using the subject ADCs may be combined with, for example, any known therapeutic agent or method for treating type 1 diabetes, optionally as described herein.

Thus, treatment of uveitis, using the subject ADCs may be combined with, for example, any known therapeutic agent or method for treating uveitis, optionally as described herein.

Thus, treatment of psoriasis using the subject ADCs may be combined with, for example, any known therapeutic agent or method for treating psoriasis, optionally as described herein.

Thus, treatment of Sjögren's syndrome, using the subject ADCs may be combined with, for example, any known therapeutic agent or method for treating for Sjögren's syndrome, optionally as described herein.

Thus, treatment of systemic lupus erythematosus, using the subject ADCs may be combined with, for example, any known therapeutic agent or method for treating for systemic lupus erythematosus, optionally as described herein.

Thus, treatment of GVHD, using the subject ADCs may be combined with, for example, any known therapeutic agent or method for treating GVHD, optionally as described herein.

Thus, treatment of chronic or acute infection and/or hepatotoxicity associated therewith, e.g., hepatitis, using the subject ADCs may be combined with, for example, any known therapeutic agent or method for treating for chronic or acute infection and/or hepatotoxicity associated therewith, optionally as described herein.

Thus, treatment of chronic or acute Severe asthma, using the subject ADCs may be combined with, for example, any known therapeutic agent or method for treating for Severe asthma, optionally as described herein.

Thus, treatment of chronic or acute Giant cell arteritis, using the subject ADCs may be combined with, for example, any known therapeutic agent or method for treating for Giant cell arteritis, optionally as described herein.

Thus, treatment of chronic or acute ANKA vasculitis, using the subject ADCs may be combined with, for example, any known therapeutic agent or method for treating for ANKA vasculitis, optionally as described herein.

Thus, treatment of chronic or acute IBD (Colitis and Crohns), using the subject ADCs may be combined with, for example, any known therapeutic agent or method for treating for ANKA vasculitis, optionally as described herein.

Again, it should be understood that the disease conditions identified herein and proposed treatments using the subject corticosteroid compounds, corticosteroid-linker compounds and ADCs containing same are intended to be exemplary and not exhaustive.

In the above-described therapies preferably a subject with one of the aforementioned or other autoimmune or inflammatory conditions will be administered an ADC according to the invention, thereby preventing or ameliorating the disease symptoms.

### Pharmaceutical Compositions

In another aspect, the present invention provides a composition, e.g., a pharmaceutical composition, containing one or a combination of ADCs or novel steroids of Formula I. II or III or corticosteroid-linker compounds according to the invention and optionally another immunosuppressive or other active agent. Thus, the present invention features a pharmaceutical composition comprising a therapeutically effective amount of ADCs or novel steroids of Formula I, II or III or corticosteroid-linker compounds containing according to the invention. In particular the present invention features a pharmaceutical composition comprising a therapeutically effective [anti-inflammatory] amount of at least one of the novel steroids of Formula I, II or III or corticosteroid-linker compounds containing or ADCs containing according to the invention.

The term “therapeutically effective amount” refers to an amount of agent according to the present invention that is effective to treat a disease or disorder in a mammal. The therapeutic agents of the present invention can be provided to the subject alone or as part of a pharmaceutical composition where they are mixed with a pharmaceutically acceptable carrier. In many instances ADCs according to the invention will be used in combination with other immunotherapeutics or other therapeutic agents useful in treating a specific condition.

A composition is said to be a “pharmaceutically acceptable” if its administration can be tolerated by a recipient patient. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion).

Such compositions include sterile water, buffered saline (e.g., Tris-HCl, acetate, phosphate), pH and ionic strength and optionally additives such as detergents and solubilizing agents (e.g., Polysorbate 20, Polysorbate 80), antioxidants (e.g., ascorbic acid, sodium metabisulfite), preservatives (e.g., Thimersol, benzyl alcohol) and bulking substances (e.g., lactose, mannitol). Non-aqueous solvents or vehicles may also be used as detailed below.

Examples of suitable aqueous and nonaqueous carriers that may be employed in the pharmaceutical compositions according to at least some embodiments of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Depending on the route of administration, the active compound, i.e., monoclonal or polyclonal antibodies and antigen-binding fragments and conjugates containing same, and/or alternative scaffolds, that specifically bind any one of VISTA proteins, or bispecific molecule, may be coated in a material to protect the compound from the action of acids and other natural conditions that may inactivate the compound. The pharmaceutical compounds according to at least some embodiments of the invention may include one or more pharmaceutically acceptable salts. A “pharmaceutically acceptable salt” refers to a salt that retains the desired biological activity of the parent compound and does not impart any undesired toxicological effects (See e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydriodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition according to at least some embodiments of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, a-tocopherol, and the like; and (3) metal chelating agents, such as citric acid, ethylenediamine tetraacetic acid (EDTA), sorbitol, tartaric acid, phosphoric acid, and the like.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions according to at least some embodiments of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

A composition of the present invention can be administered via one or more routes of administration using one or more of a variety of methods known in the art. As will be appreciated by the skilled artisan, the route and/or mode of administration will vary depending upon the desired results. Preferred routes of administration for therapeutic agents according to at least some embodiments of the invention include intravascular delivery (e.g. injection or infusion), intravenous, intramuscular, intradermal, intraperitoneal, subcutaneous, spinal, oral, enteral, rectal, pulmonary (e.g. inhalation), nasal, topical (including transdermal, buccal and sublingual), intravesical, intravitreal, intraperitoneal, vaginal, brain delivery (e.g. intra-cerebroventricular, intracerebral, and convection enhanced diffusion), CNS delivery (e.g. intrathecal, perispinal, and intra-spinal) or parenteral (including subcutaneous, intramuscular, intravenous and intradermal), transmucosal (e.g., sublingual administration), administration or administration via an implant, or other parenteral routes of administration, for example by injection or infusion, or other delivery routes and/or forms of administration known in the art. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In a specific embodiment, a protein, a therapeutic agent or a pharmaceutical composition according to at least some embodiments of the present invention can be administered intraperitoneally or intravenously.

Alternatively, an ADC according to the invention can be administered via a non-parenteral route, such as a topical, epidermal or mucosal route of administration, for example, intranasally, orally, vaginally, rectally, sublingually or topically.

Therapeutic compositions comprising ADCs according to the invention can be administered with medical devices known in the art. For example, in a preferred embodiment, a therapeutic composition according to at least some embodiments of the invention can be administered with a needles hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the ADCs can be formulated to ensure proper distribution in vivo. For example, the blood-brain barrier (BBB) excludes many highly hydrophilic compounds. To ensure that the therapeutic compounds according to at least some embodiments of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J Physiol. 1233:134); pl20 (Schreier et al. (1994) J. Biol. Chem. 269:9090); See also K. Keinanen; M. L. Laukkanen (1994) FEBS Lett. 346:123; J. J. Killion; and I. J. Fidler (1994) Immunomethods 4:273.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., ADC according to the invention, may be coated in a material to protect the compound from the action of acids and other natural conditions that may inactivate the compound. The pharmaceutical compounds according to at least some embodiments of the present invention may include one or more pharmaceutically acceptable salts.

A “pharmaceutically acceptable salt” refers to a salt that retains the desired biological activity of the parent compound and does not impart any undesired toxicological effects (See e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition according to at least some embodiments of the present invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, a-tocopherol, and the like; and (3) metal chelating agents, such as citric acid, ethylenediamine tetraacetic acid (EDTA), sorbitol, tartaric acid, phosphoric acid, and the like. Examples of suitable aqueous and nonaqueous carriers that may be employed in the pharmaceutical compositions according to at least some embodiments of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions according to at least some embodiments of the present invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient, i.e., ADC or novel steroid compound according to the invention, which can be combined with a carrier material to produce a single dosage form will vary depending upon the subject being treated, and the particular mode of administration. The amount of active ingredient, i.e., ADC or novel steroid compound according to the invention, which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about I percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms according to at least some embodiments of the present invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the ADC disclosed herein, in some embodiments the dosage ranges will generally comprise administration of an amount of the ADC which delivers the same or lesser amount of the anti-inflammatory agent, e.g., a steroid of formula I, II or III, for therapeutic efficacy compared to that required for efficacy of conventional steroid compounds such as such as dexamethasone or budesonide when administered via conventional routes, i.e., wherein the steroid is administered in naked or unconjugated form to treat the specific condition. In exemplary embodiments the dosage ranges will generally comprise administration of an amount of the ADC which delivers a reduced amount of the anti-inflammatory agent, e.g., from 10-90% thereof, e.g., of dexamethasone or budesonide, for therapeutic efficacy than if the A1 were administered via conventional routes, i.e., wherein the steroid is administered in naked or unconjugated form to treat the specific condition, as it is anticipated based on the results obtained to date that the present ADCs, aside from reducing or eliminating adverse side effects of the A1 such as a steroid, will be more effectively delivered to the desired target immune cells and will be less prone to reach non-target cells, thereby reducing the required dosage effective amount of the steroid and/or reducing effects non non-target cells.

The ADCs disclosed herein can be administered on multiple occasions. Intervals between single dosages can be, for example, every 3-5 days, weekly, bi-weekly, every 2-3 weeks, etc. In some methods, the dosage is adjusted to achieve a plasma steroid concentration of a desired level. Determining an effective dosing regimen for treatment or prophylaxis using the subject ADCs should be relatively facile compared to other ADCs wherein the antibody therein elicits a biologic or therapeutic effect as the therapeutic activity of the subject ADCs in some instances is entirely governed by the anti-inflammatory payload. (Essentially, in some instances the antibody only targets and directs internalization of the subject ADCs into specific immune cells and itself elicits no effect on immunity).

Alternatively, the ADC can be administered as a sustained release formulation, in which case less frequent administration is required. The dosage and frequency of administration can vary depending on whether the treatment is prophylactic or therapeutic. In prophylactic applications, a relatively low dosage may be administered at relatively infrequent intervals over a long period of time. Some patients may continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime. As mentioned the subject ADCs are preferred for such uses as they remain in the peripheral circulation for a very short duration, do not bind to non-immune cells and do not appreciably elicit toxicity to non-target cells.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

### Exemplary Embodiments

This invention provides antibody drug conjugates (ADC's) that comprise an antibody or antigen binding fragment comprising an antigen binding region that specifically binds to an immune cell antigen, e.g., human V-domain Ig Suppressor of T cell Activation (human VISTA) (“A”), a cleavable and/or non-cleavable linker (“L”) and at least one small molecule anti-inflammatory agent (“AI”), optionally “Q”, a heterobifunctional group” or “heterotrifunctional group” which is a chemical moiety optionally used to connect the linker to the anti-VISTA antibody or antibody fragment and at least one small molecule anti-inflammatory agent (“A1”) (typically a steroid), said ADC being represented by the formula:

“A-(Q-L-AI) n” or “(AI-L-Q) n-A” wherein “n” is at least 1 and may be as high as 12 or more, and wherein the ADC, or composition containing, when administered to a subject in need thereof, is preferentially delivered to cells that express the antigen bound by the antibody, e.g., VISTA expressing immune cells, optionally monocytes, myeloid cells, T cells, Tregs, eosinophils, CD4 or CD8 T cells, neutrophils; and results in the functional internalization of the small molecule anti-inflammatory agent into said immune cells, optionally at physiological conditions (≈pH 7.5), preferably wherein the antibody or antibody fragment, e.g., an anti-VISTA antibody or antigen binding fragment, has a short in vivo serum half-life in serum at physiological pH (˜pH 7.5), optionally an in vivo serum half-life in serum at physiological pH (˜pH 7.5) in a rodent (human VISTA knock-in mouse or rat) of no more than about 70 hours, no more than about 60 hours, no more than 50 hours, no more than 40 hours, no more than 30 hours, no more than 24 hours, no more than 22-24 hours, no more than 20-22 hours, no more than 18-20 hours, no more than 16-18 hours, no more than 14-16 hours, no more than 12-14 hours, no more than 10-12 hours, no more than 8-10 hours, no more than 6-8 hours, no more than 4-6 hours, no more than 2-4 hours, no more than 1-2 hours, no more than 0.5 to 1.0 hours, or no more than 0.1-0.5 hours and/or in a primate (e.g., human or Cynomolgus macaque) of no more than about 3, 2.5, or 2.3±0.7 days.

Exemplary cleavable and non-cleavable linkers which may be incorporated into the subject ADCs have been previously identified herein and are well known in the art. Specific types and examples of such types of linkers which may be used in ADCs according to the invention are further identified below.

As mentioned, while steroid compounds are very efficacious at inhibiting inflammation associated with different conditions such as autoimmune, allergic and inflammatory disorders, cancer and infectious diseases, their utility in the chronic treatment of disease is limited due to severe side effects which will be alleviated when they are incorporated into ADCs according to the invention.

Particularly the invention includes ADCs according to the invention wherein the A1 comprises a steroid (glucocorticoid agonist) which comprises the following generic structures:

Specifically provided herein are glucocorticoid agonist compounds having the following structure of Formula (I):

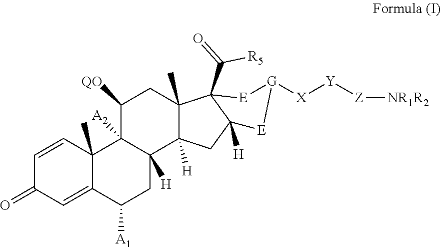

wherein X is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo[2.2.2]octane, adamantane, and cubane each of which can be substituted with 1-4 heteroatoms independently selected from F, Cl, Br, I, N, S, and O, each of which ring structure may contain at least one skeletal heteroatom selected from N, S, and O, and are optionally further substituted with 1-4 C1-3 alkyl or C1-3 perfluoroalkyl;
Z is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo[2.2.2]Octane, Adamantane, and Cubane Each of which can be Substituted with 1-4 heteroatoms independently selected from F, Cl, Br, I, N, S, and O, each of which ring structure may contain at least one skeletal heteroatom selected from N, S, and O, and are optionally further substituted with 1-4 C₁₋₃ alkyl or C₁₋₃ perfluoroalkyl;
Y is selected from CHR₁, O, S, and NR₁;
E is selected from CH₂ and O;
G is selected from CH, and N;
further wherein when G is CH and X is phenyl, Z is not phenyl;
the linkage of G to X may optionally be selected from C₁₋₃ alkyl and ethylene oxide, each of which may be substituted with 1-4 heteroatoms independently selected from N, S, and O and are optionally further substituted with 1-4 C₁₋₃ alkyl;
the linkage of X to Z may occupy any available position on X and Z;
substituent NR₁R₂ may occupy any available position on Z;
R₁ is selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylaminoC(O)—;
when R₁ is H, R₂ may be selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylaminoC(O)—;
when R₁ is H, linear or branched alkyl of 1-8 carbons, or heteroaryl, R2 may be a functional group selected from

[(C═O)CH(W)NH]ₘ—[C═O]—[V]ₖ-J,

(C═O)OCH₂-p-aminophenyl-N—V-J,

(C═O)OCH₂-p-aminophenyl-N—[(C═O)CH(W)NH]ₘ—[C═O]—[V]ₖ-J, and

[V]ₖ—(C═O)OCH₂-p-aminophenyl-N—[(C═O)CH(W)NH]ₘ—[C═O]-J,

wherein m=1-6, k=0-1, and each permutation of W may independently be selected from H, [(CH₂)ₙR₃] where n=1-4, a branched alkyl chain terminating in R₃, and a linear or branched polyethylene oxide group comprising 1-13 units;
R₃ is selected from H, methyl, ethyl, isopropyl, OH, O-alkyl, NH2, NH-alkyl, N-dialkyl, SH, S-alkyl, guanidine, urea, carboxylic acid, carboxamide, carboxylic ester, substituted or unsubstituted aryl, substituted or unsubstituted heteroaryl, wherein said aryl and heteroaryl substituents may be selected from alkyl, haloalkyl, halogen, biphenyl, nitro, nitrile, —OH, —O-alkyl, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)—, and dialkylaminoC(O)—;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH₂, N₃, thio, cyclooctyne, —OH, —CO₂H, trans-cyclooctene, alkynyl, propargyl,

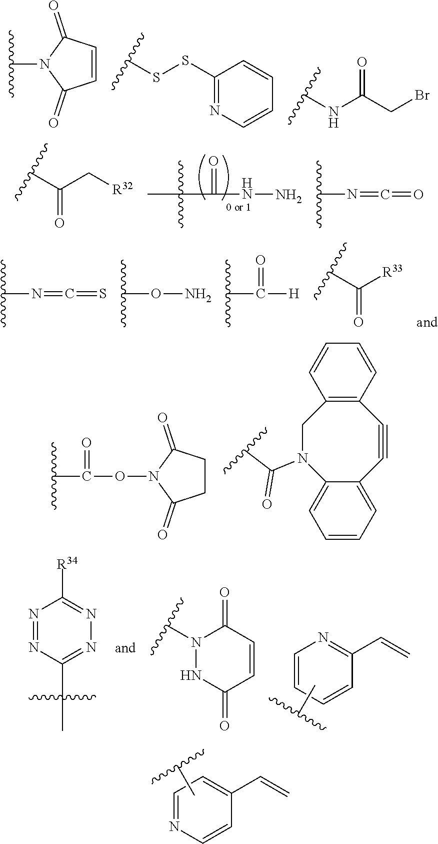

where R₃₂ is selected from Cl, Br, F, mesylate, and tosylate and R₃₃ is selected from Cl, Br, I, F, OH, —O—N-succinimidyl, —O-(4-nitrophenyl), —O-pentafluorophenyl or —O-tetrafluorophenyl R₃₄ is H, Me, tetrazine-H, and tetrazine-Me;
R₅ is selected from the group consisting of —CH₂OH, —CH₂SH, —CH₂Cl, —SCH₂Cl, —SCH₂F, —SCH₂CF₃, hydroxy, —OCH₂CN, —OCH₂Cl, —OCH₂F, —OCH₃, —OCH₂CH₃, —SCH₂CN, and

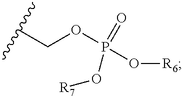

R₆ and R₇ are independently selected from hydrogen and C1-10 alkyl;
Q may be H,

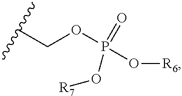

C(O)R₈ where R₈ is linear or branched alkyl of 1-8 carbons, or (C═O)NR₄CHₙNR₄ (C═O)OCH₂—(V)ₙ-J where n=1-4 and R₄=H, alkyl or branched alkyl, or P(O)OR₄;
A₁ and A₂ are independently selected from H and F; and unless otherwise specified, all possible stereoisomers are claimed, and further optionally, X and Z are independently selected from phenyl, spiro[3.3]heptane, [1.1.1]bicyclopentane, and bicyclo[2.2.2]octane; Y is selected from CH₂ and O; permutations of W are independently selected from CH₂CH₂CO₂H and H, and further wherein when G is CH and X is phenyl, Z is not phenyl;
   OR
glucocorticoid agonist compounds which possess the structure of Formula (II):

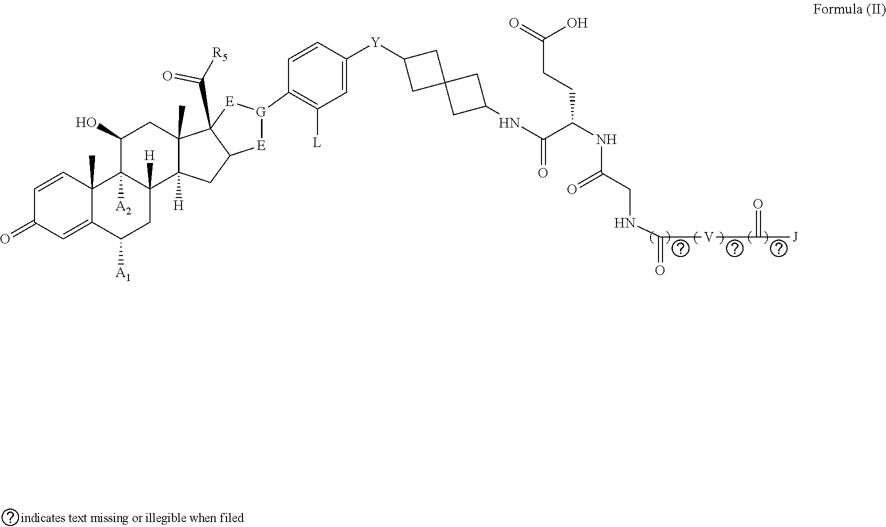

wherein
Y is selected from CH₂ and O;
E is selected from CH₂ and O;
G is selected from CH, and N;
L is selected from H and F;
R₅ is selected from —CH₂OH, —SCH₂F and

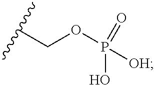 [SMILES: *COP(=O)(O)O]

A₁ and A₂ are independently selected from H and F;

V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH₂, N₃, thio, cyclooctyne, —OH, —CO₂H, trans-cyclooctene, alkynyl, propargyl,

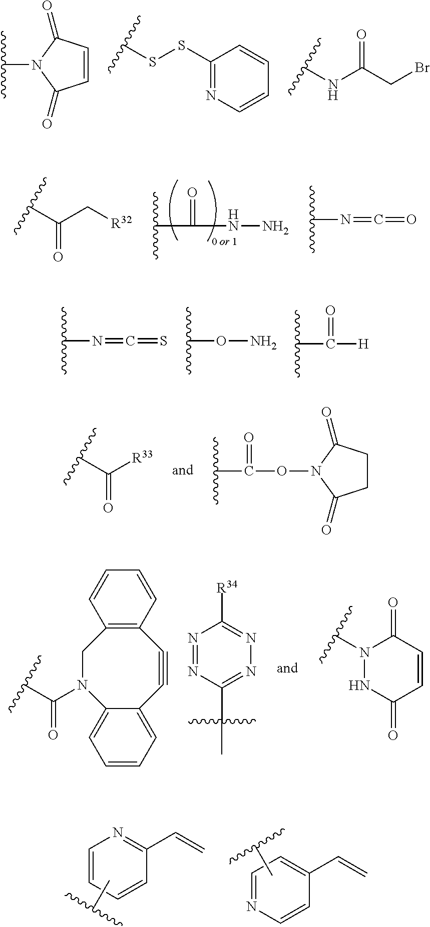

where R₃₂ is selected from Cl, Br, F, mesylate, and tosylate and R₃₃ is selected from Cl, Br, I, F, OH, —O—N-succinimidyl, —O-(4-nitrophenyl), —O-pentafluorophenyl or —O-tetrafluorophenyl R₃₄ is H, Me, tetrazine-H, and tetrazine-Me;
OR
glucocorticoid agonist compounds which possess the structure of Formula (III):

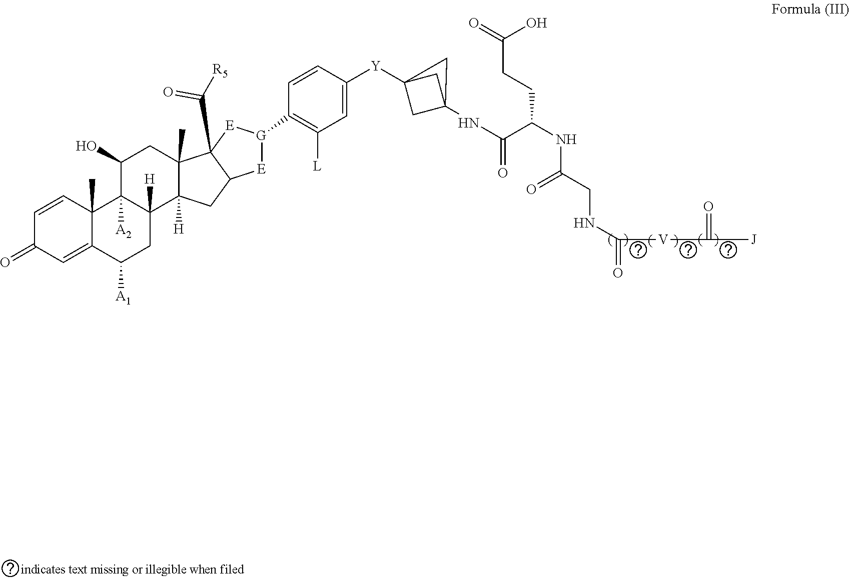

wherein
Y is selected from CH2 and O;
E is selected from CH2 and O;
G is selected from CH, and N;
L is selected from H and F;
R₅ is selected from —CH₂OH, —SCH₂F and

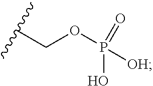 [SMILES: *COP(=O)(O)O]

A₁ and A₂ are independently selected from H and F;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH₂, N₃, thio, cyclooctyne, —OH, —CO₂H, trans-cyclooctene, alkynyl, propargyl,

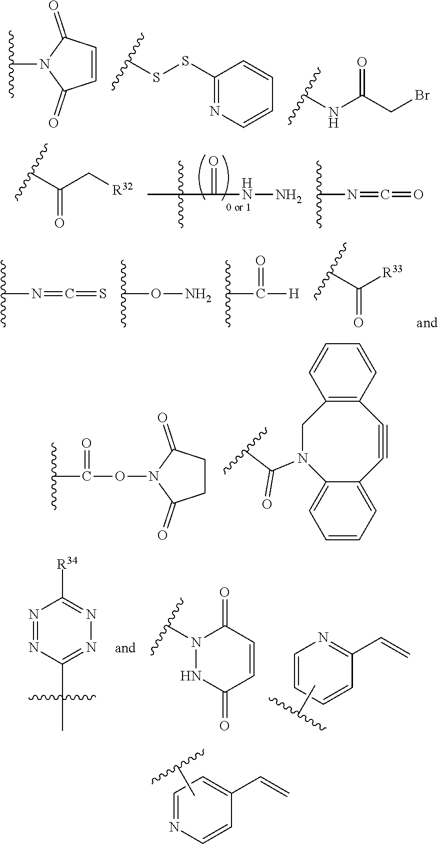

where R₃₂ is selected from Cl, Br, F, mesylate, and tosylate and R₃₃ is selected from Cl, Br, I, F, OH, —O—N-succinimidyl, —O-(4-nitrophenyl), —O-pentafluorophenyl or —O-tetrafluorophenyl R₃₄ is H, Me, tetrazine-H, and tetrazine-Me.

### I. EXEMPLARY LINKERS

As mentioned different linkers may be incorporated into ADCs according to the invention. Such linkers have been previously identified in the definition section wherein a “linker” was defined. Additionally, exemplary linkers which may be incorporated into ADCs according to the invention are identified below:

### A. Immolative Linker ADCs

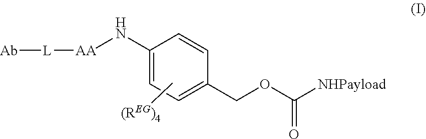

Ab=Antibody, preferably an antibody that binds to human immune cells, preferably an anti-VISTA antibody that binds to human VISTA immune cells at physiologic pH;
L=Linker;
AA=Single, double, or triple amino acid sequence;

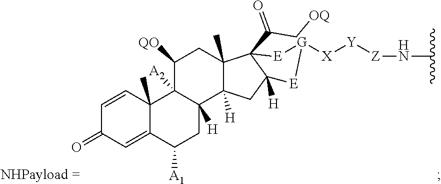

R^{EG} is independently selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—;

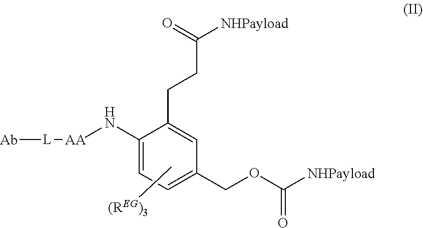

Ab=Antibody;
L=Linker;
AA=Single, double, or triple amino acid sequence;

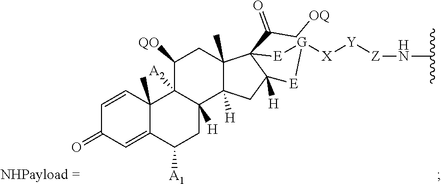

R^{EG} is independently selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—;

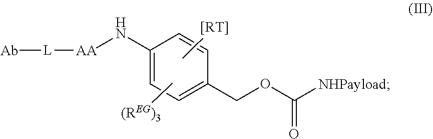

Ab=Antibody;
L=Linker,
AA=Single, double, or triple amino acid sequence or not present;

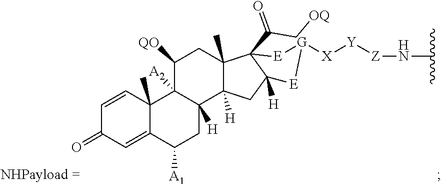

REG is independently selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—;

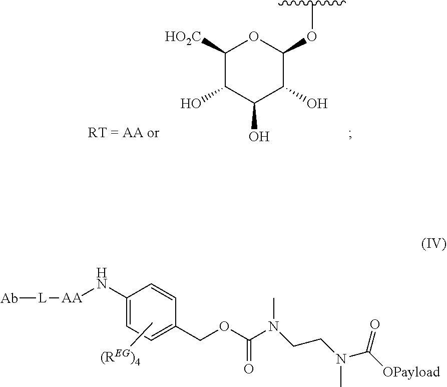

Ab=Antibody, optionally an anti-human VISTA antibody;
L=Linker;
AA=Single, double, or triple amino acid sequence;

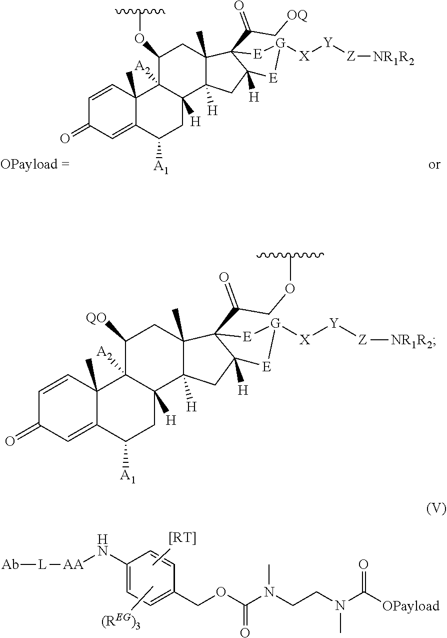

Ab=Antibody;
L=Linker;
AA=Single, double, or triple amino acid sequence or not present;

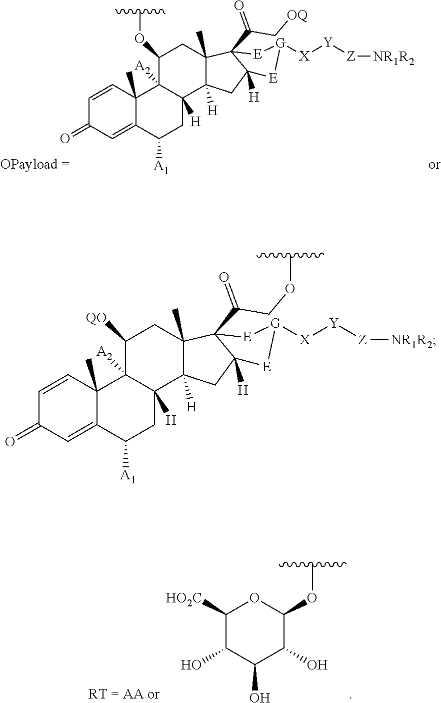

### B. Amino Acid (AA) Linkers

### (I) Sequences Cleaved by Cathepsins

a. Single Amino Acid Linkers

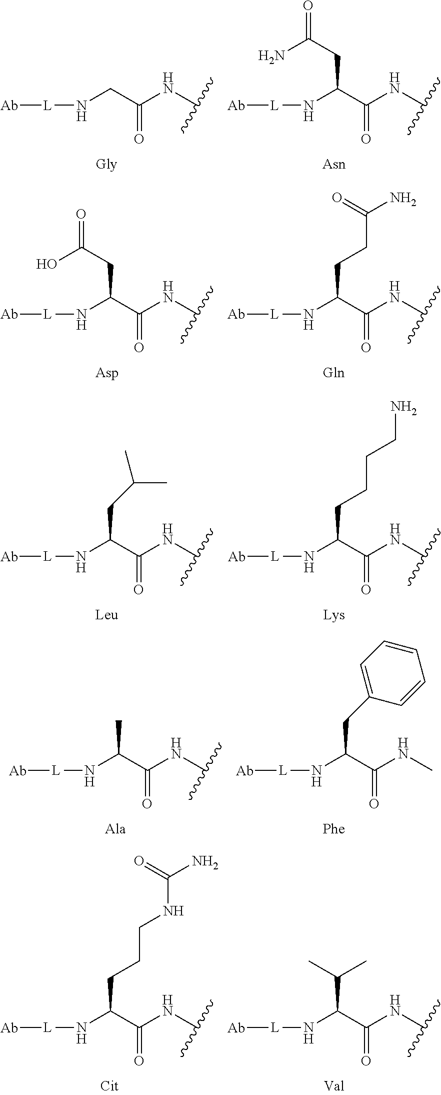

b. Dipeptide Linkers

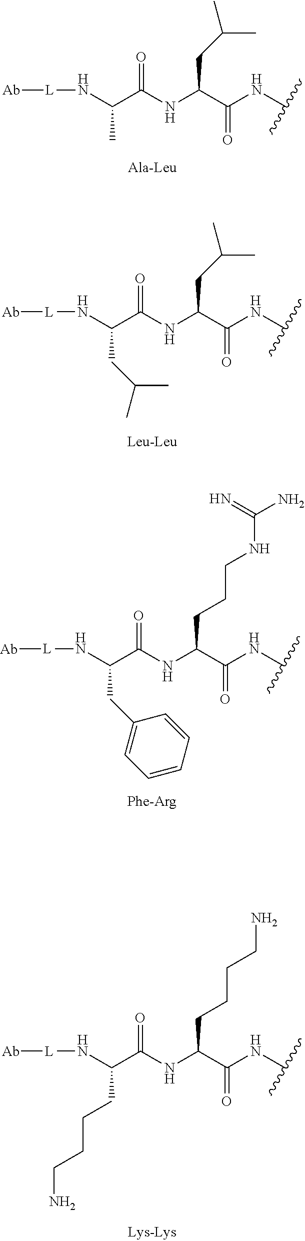

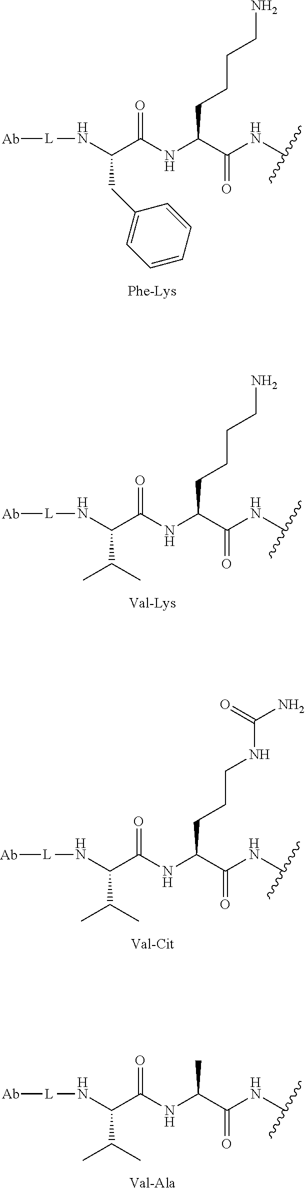

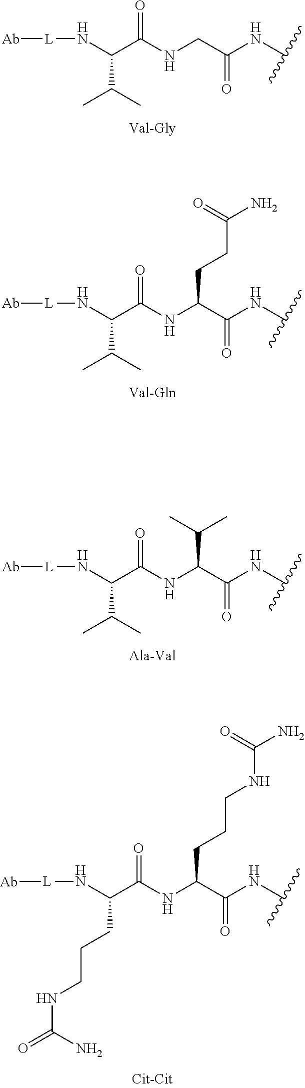

c. Tripeptide Linkers

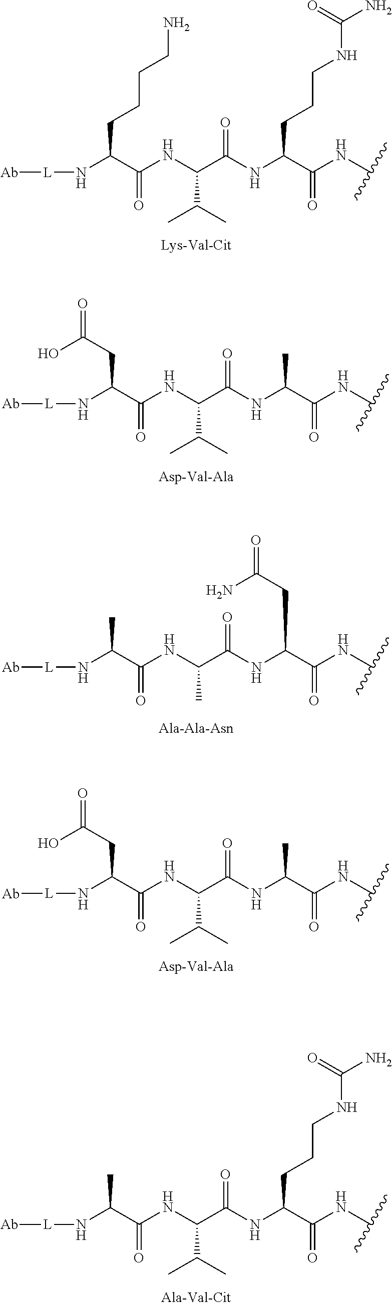

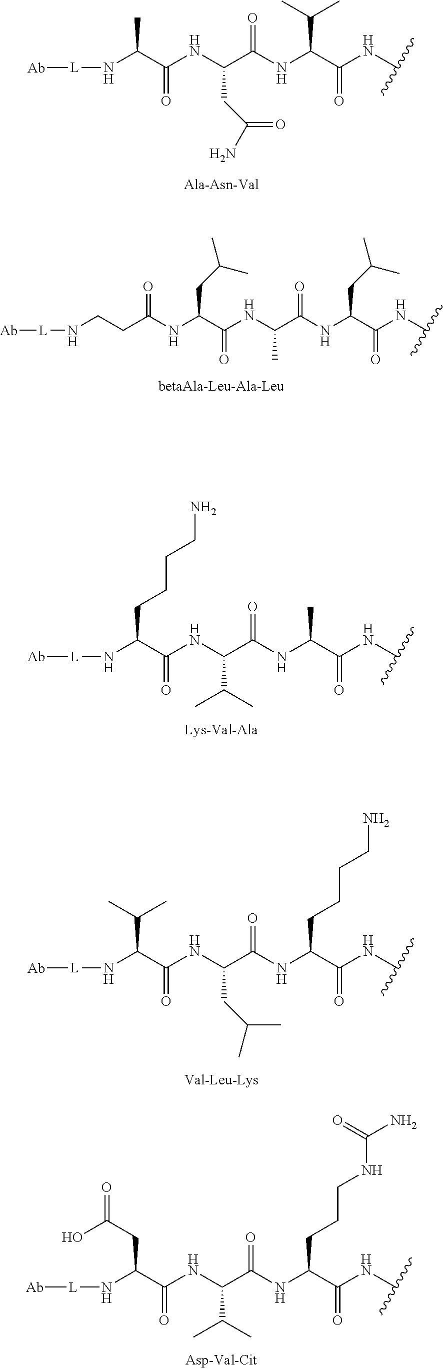

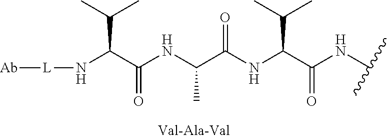

### (I) Legumain Cleavable Linkers

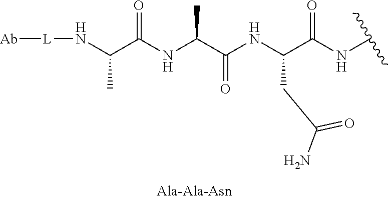

where,
L=linker
Ab=antibody

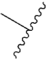

indicates point of attachment to the payload or an immolative linker.

### II. EXEMPLARY ANTIBODY CONJUGATION STRATEGIES

Different conjugation strategies may be used to conjugate the anti-VISTA antibody to the linker and payload (steroid or other anti-inflammatory compound). Detailed synthetic methods for producing exemplary ADCs and linker payloads are provided in the examples. Additionally, exemplary conjugation strategies are provided below:

### (I) Payload-Linker-J

Where payload is:

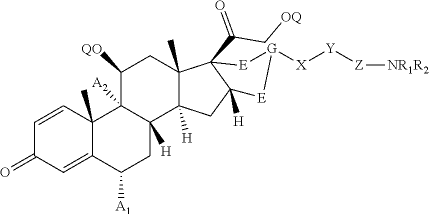

Linker=Q, R1 or R2
J is a functional group suitable for reacting with a complementary functional group on an Ab to form the antibody-drug conjugate.
J is selected from:

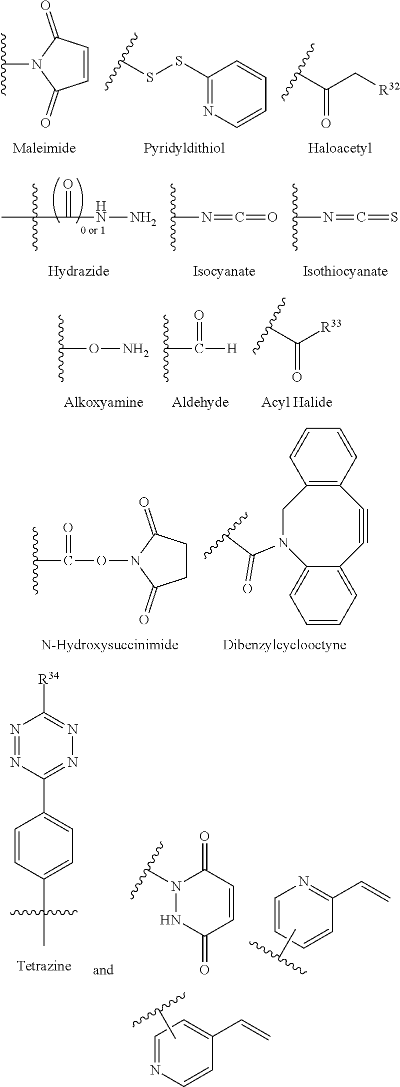

indicates a point of attachment of J to the linker selected from Q, R₁ or R₂.
where R₃₂ is Cl, Br, F, mesylate, or tosylate, R₃₃ is Cl, Br, I, F, OH, —O—N-succinimidyl, —O-(4-nitrophenyl), —O-pentafluorophenyl, or —O-tetrafluorophenyl and R₃₄ is H, Me or pyridyl;
—OH group can be esterified with a carboxy group on the antibody, for example, on an aspartic or glutamic acid side chain;
—CO2H group can be esterified with a —OH group or amidated with an amino group (for example on a lysine side chain) on the antibody;

N-hydroxysuccinimide group is functionally an activated carboxyl group and can conveniently be amidated by reaction with an amino group (e.g., from lysine);

A maleimide group can be conjugated with an —SH group on the antibody (e.g., from cysteine or from the chemical modification of the antibody to introduce a sulfhydryl functionality), in a Michael addition reaction;

Where an antibody does not have a cysteine, —SH, available for conjugation, an ¿-amino group in the side chain of a lysine residue can be reacted with 2-iminothiolane or N-succinimidyl-3-(2-pyridyldithio) propionate (“SPDP”) to introduce a free thiol (—SH) group-creating a cysteine surrogate. The thiol group can react with a maleimide or other nucleophile acceptor group to effect conjugation.

An antibody Ab can be modified with 4-(N-Maleimidomethyl) cyclohexanecarboxylic acid N-hydroxysuccinimide ester (“SMCC”) or its sulfonated variant sulfo-SMCC, both of which are available from Sigma-Aldrich, to introduce a maleimide group thereto. Then, conjugation can be effected with a drug-linker compound having an —SH group on the linker.

Copper-free “click chemistry,” in which an azide group (—N3) adds across a strained cyclooctyne to form an 1,2,3-triazole ring. The azide can be located on the antibody and the cyclooctyne on the drug-linker moiety, or vice-versa. A preferred cyclooctyne group is dibenzocyclooctyne (DBCO).

Introducing a non-natural amino acid into an antibody, with the non-natural amino acid providing a functionality for conjugation with a reactive functional group in the drug moiety. For instance, the non-natural amino acid p-acetylphenylalanine can be incorporated into an antibody or other polypeptide. The ketone group in p-acetylphenyalanine can be a conjugation site via the formation of an oxime with a hydroxylamino group on the linker-drug moiety. Alternatively, the non-natural amino acid p-azidophenylalanine (or p-azidomethyl-I-phenylalanine) can be incorporated into an antibody to provide an azide functional group for conjugation via click chemistry with DBCO to form a 1,2,3-triazole ring.

Another example would be the incorporation of an unnatural amino acid containing strained alkenes norbornene, trans-cyclooctene or cyclopropene which can undergo inverse electron demad Diels Alder “click chemistry” reaction with tetrazine to form a bicyclic diazine product.

Another conjugation technique uses the enzyme transglutaminase (preferably bacterial transglutaminase from Streptomyces mobaraensis or BTG). BTG forms an amide bond between the side chain carboxamide of a glutamine (the amine acceptor) and an alkyleneamino group (the amine donor), which can be, for example, the E-amino group of a lysine or a 5-amino-n-pentyl group. In a typical conjugation reaction, the glutamine residue is located on the antibody, while the alkyleneamino group is located on the linker-drug moiety.

### III. EXEMPLARY ANTIBODY CONJUGATES

ADC conjugates according to the invention optionally comprising an antibody or fragment which targets a desired immune cell antigen, e.g., an anti-VISTA antibody (which optionally binds to human VISTA at physiologic pH and which has a short PK as defined previously), one or more cleavable and/or non-cleavable linkers and one or more payloads (steroid or other anti-inflammatory compound) optionally attached to an immolative linker may be produced using detailed synthetic methods above-described and as disclosed in the examples. Some exemplary ADC structures and conjugation methods are provided below:

### (I) Preferred Examples

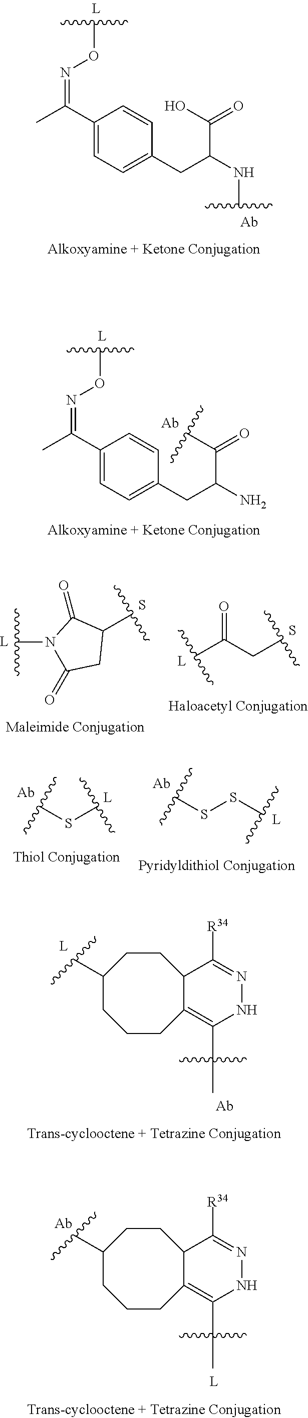

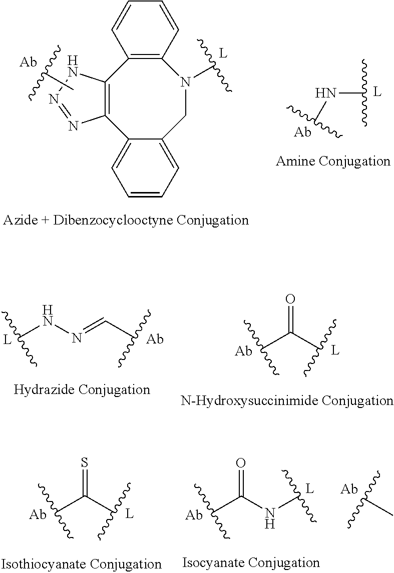

indicates a point of attachment to the antibody, or an antigen binding fragment thereof

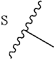

indicates a point of attachment to the antibody, or an antigen binding fragment thereof, via a sulfur atom of a cysteine residue; or a pharmaceutically acceptable salt, tautomer, stereoisomer, and/or mixture of stereoisomers thereof.

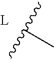

indicates a point of attachment to the linker or AA

### IV. EXEMPLARY PAYLOAD-LINKER STRUCTURES

Different payloads (steroid or other anti-inflammatory compound) attached to a linker may be produced using detailed synthetic methods above-described and as disclosed in the examples. Exemplary payload-linker structures are provided below as well as in FIG. 118A-O:

### (I) Payload-Linker-J

Where,
Linker=Protease cleavable sequence (AA)
J=Alkoxyamine

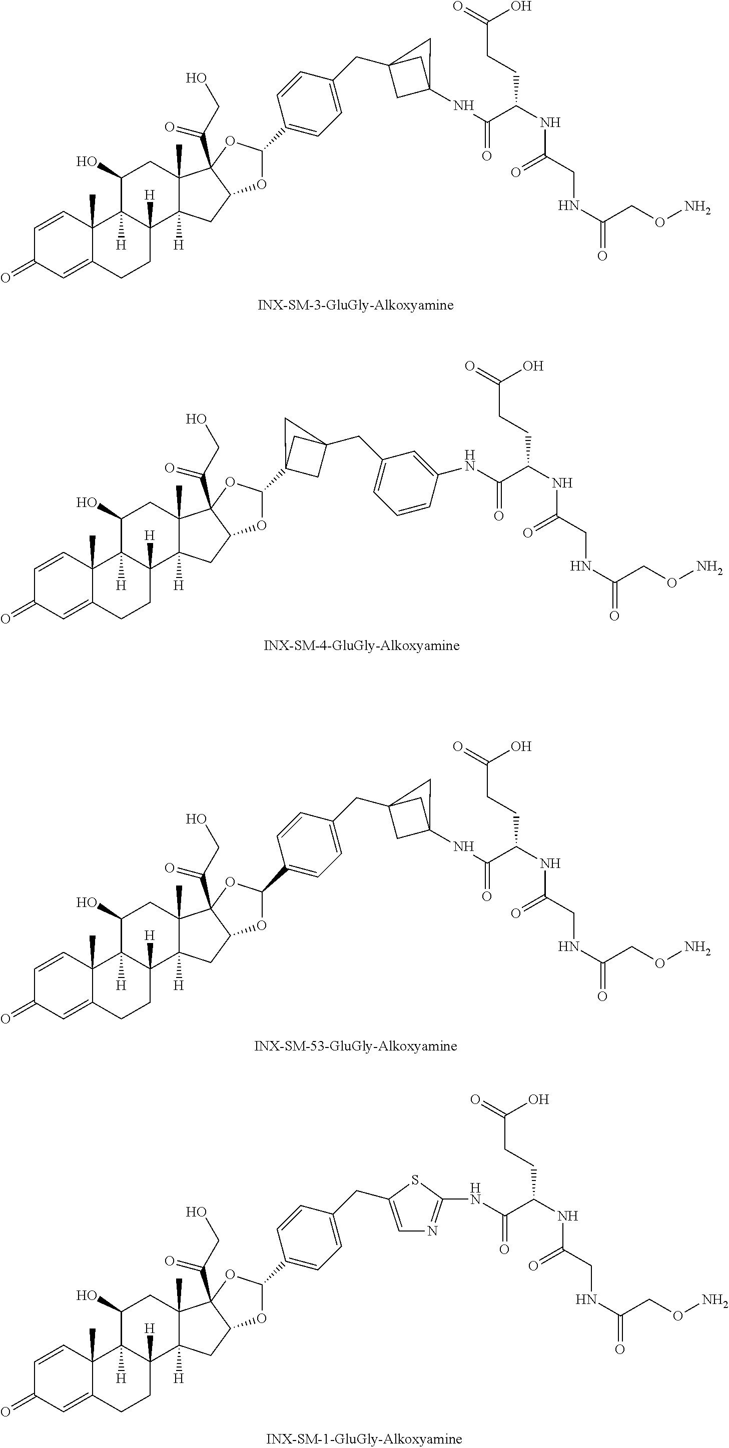

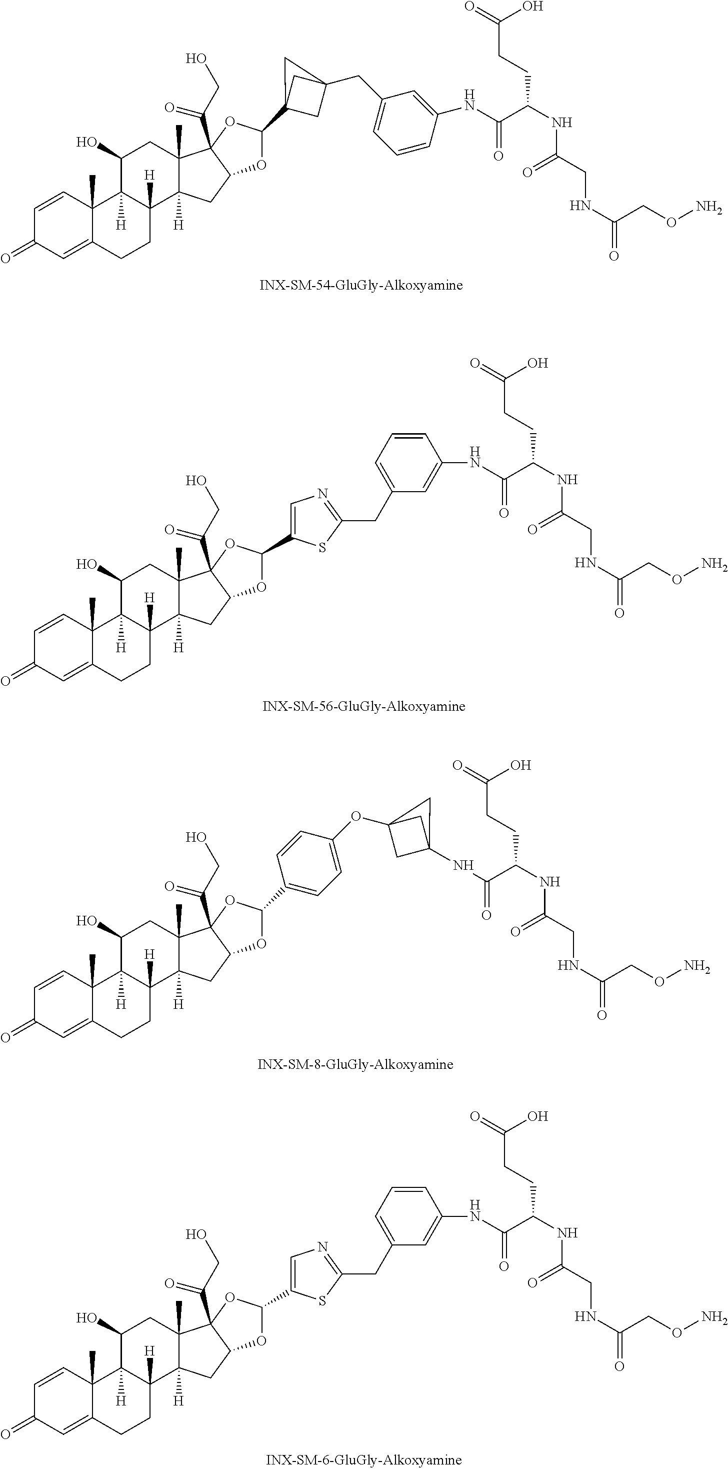

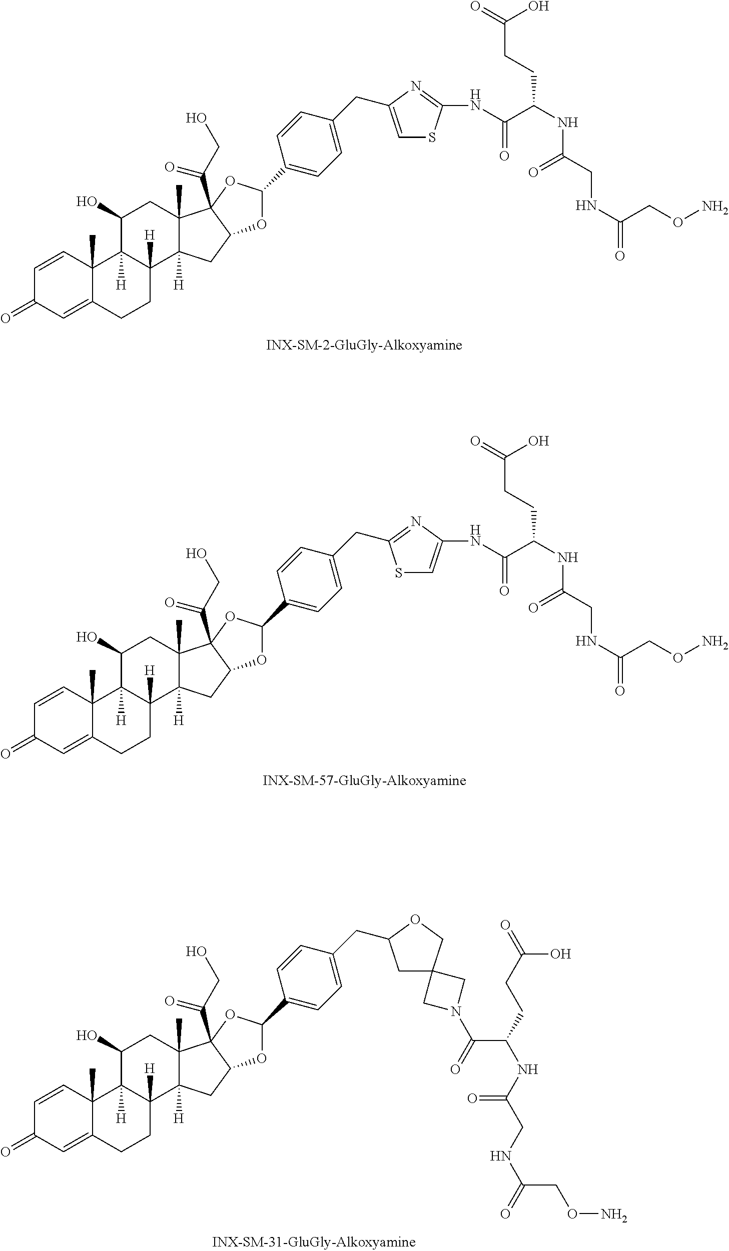

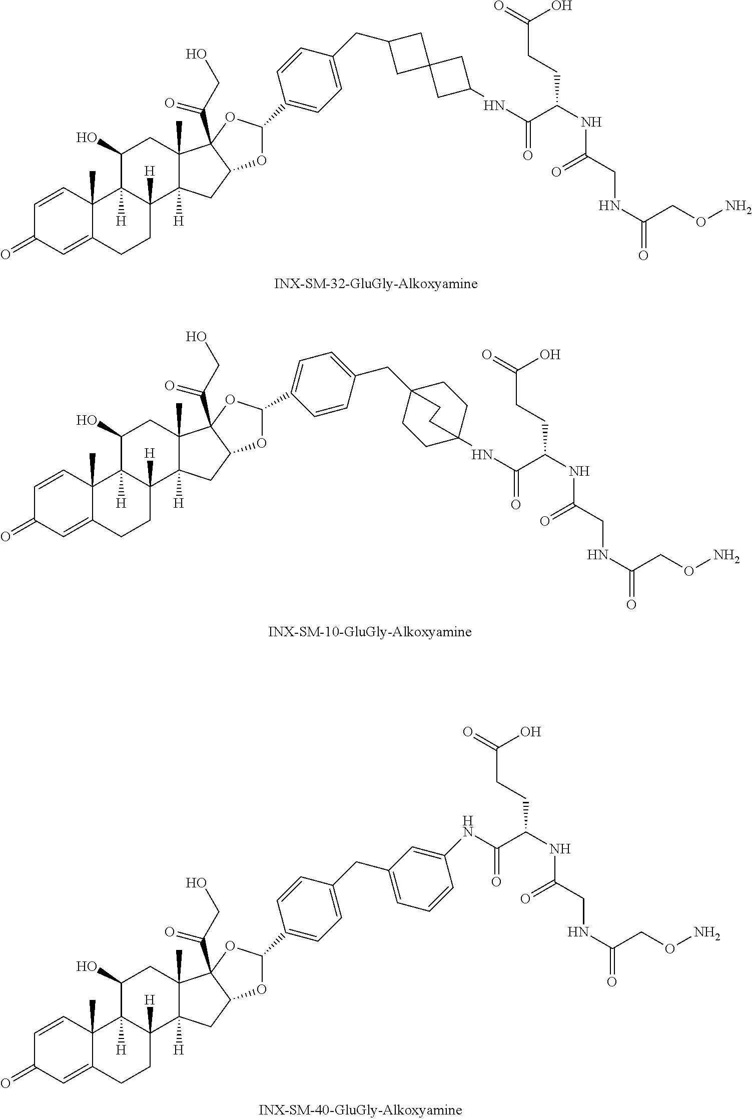

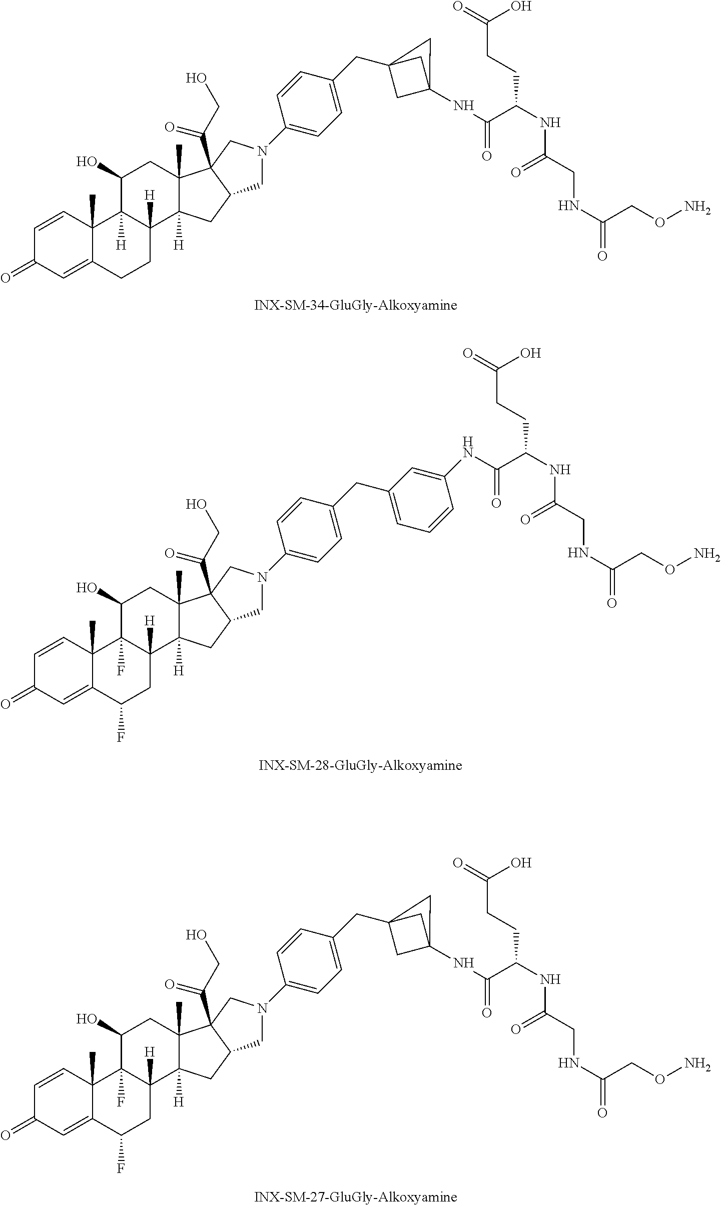

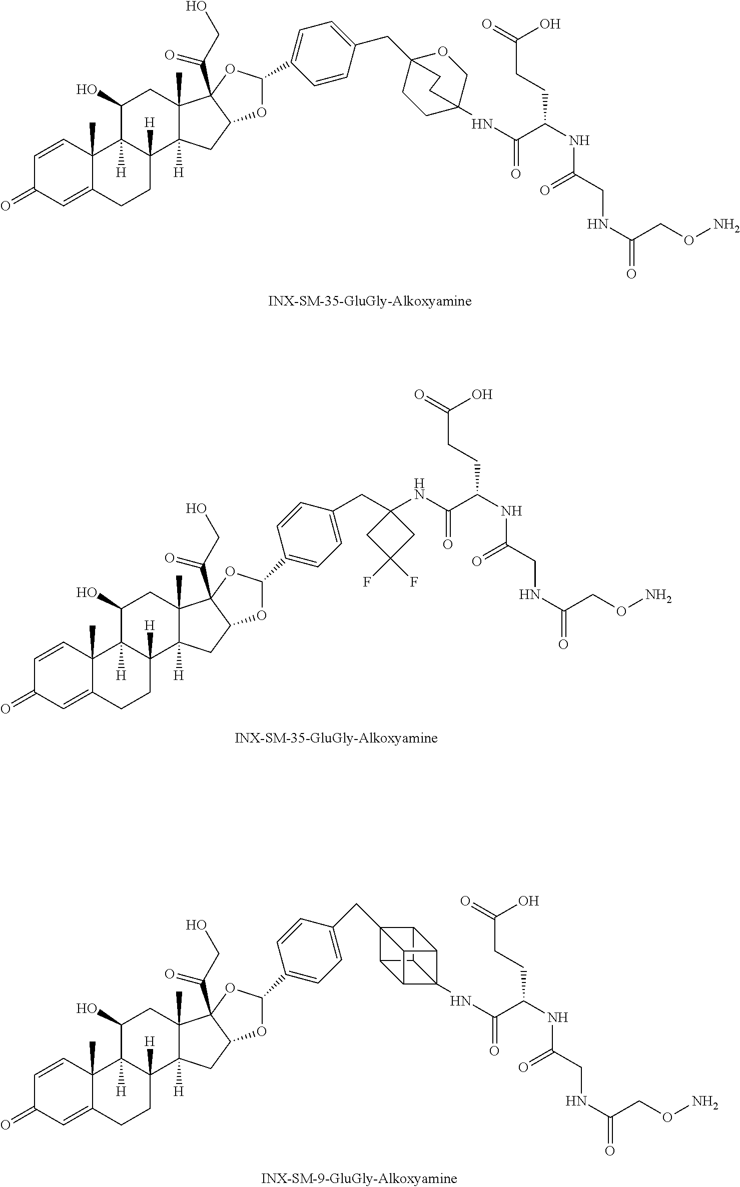

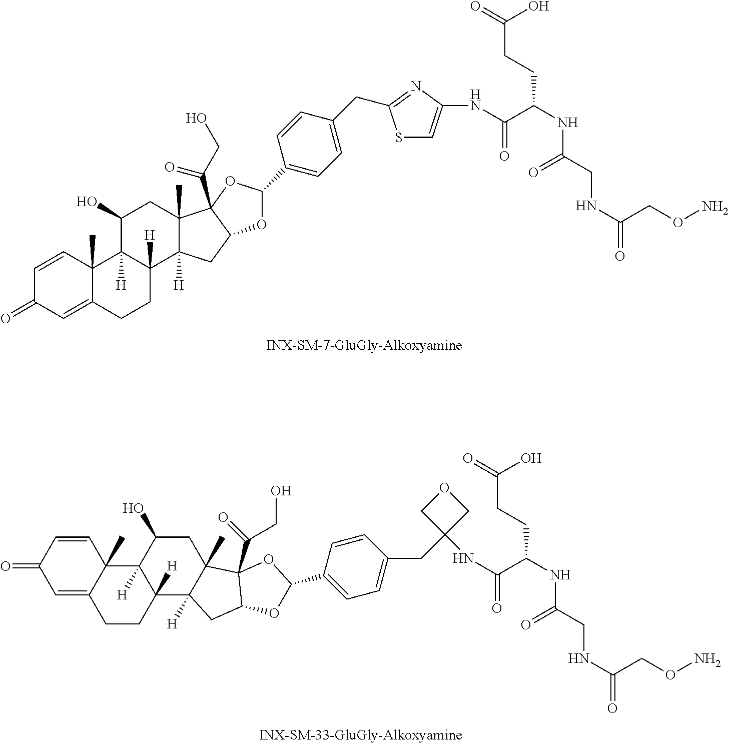

Where,
Linker=Protease cleavable sequence (AA)
J=Bromoacetyl

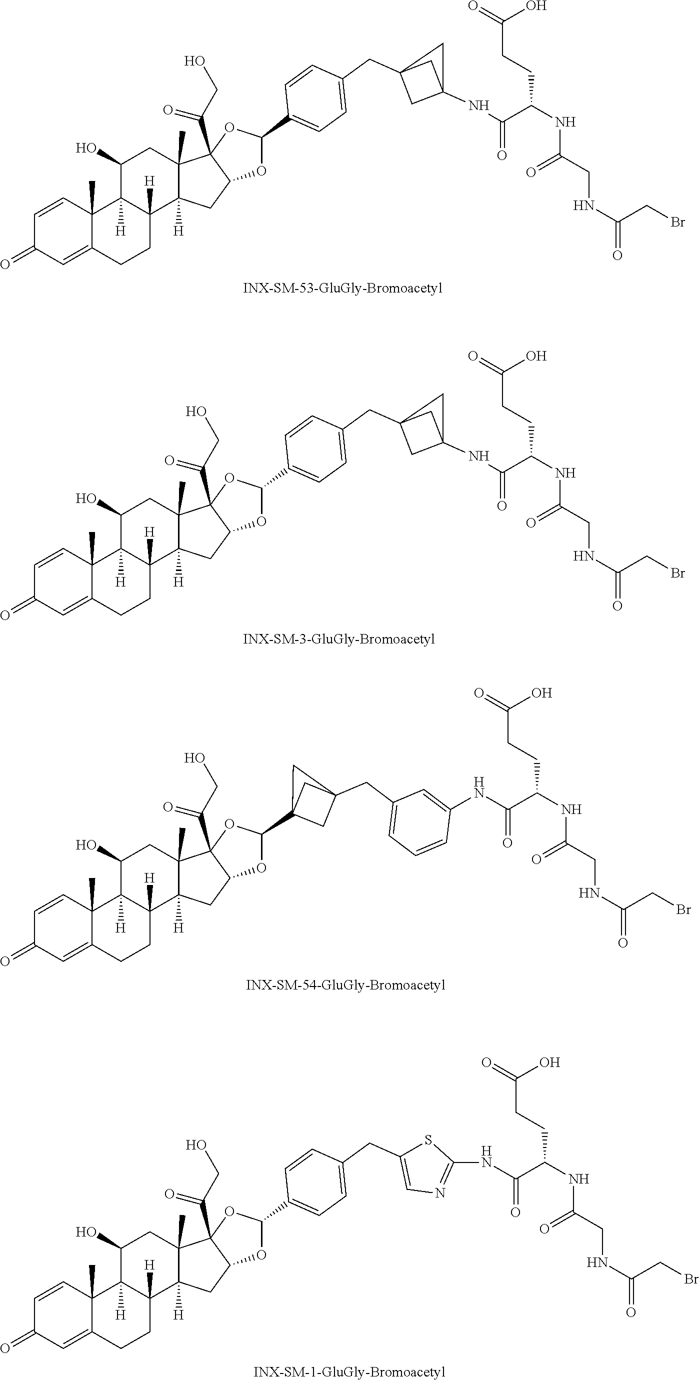

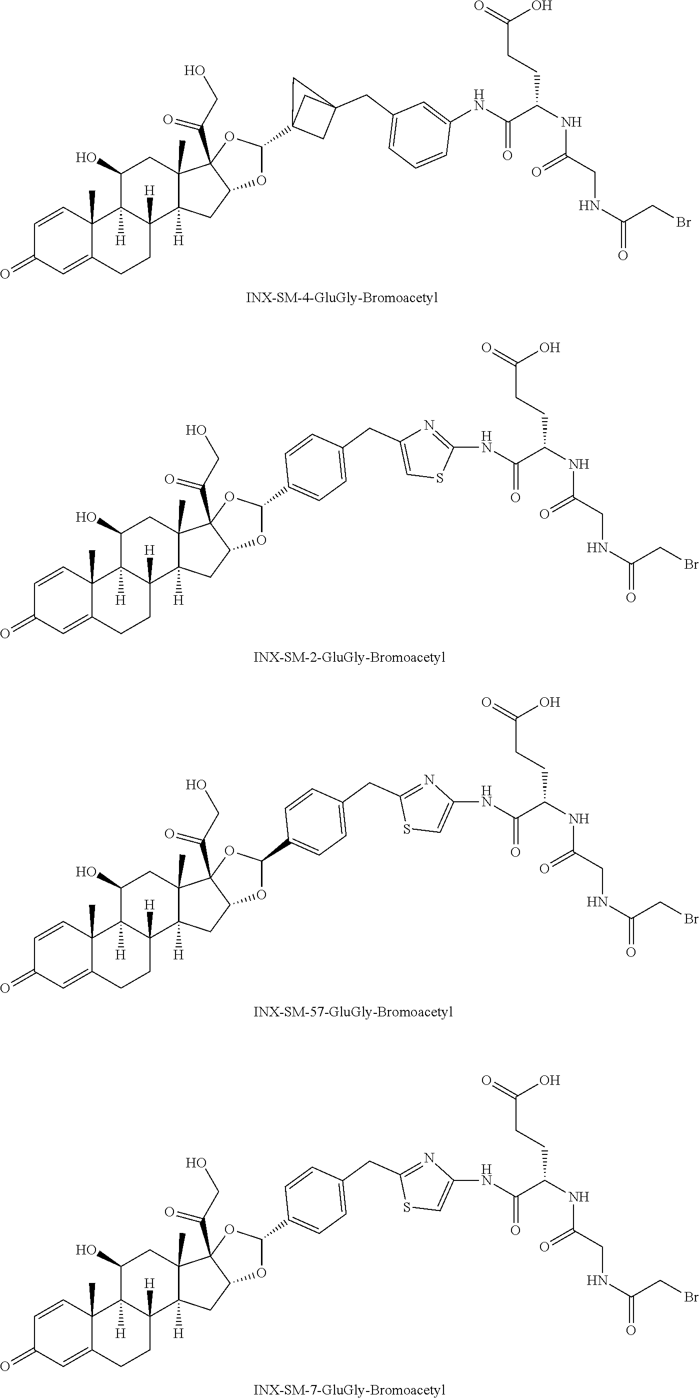

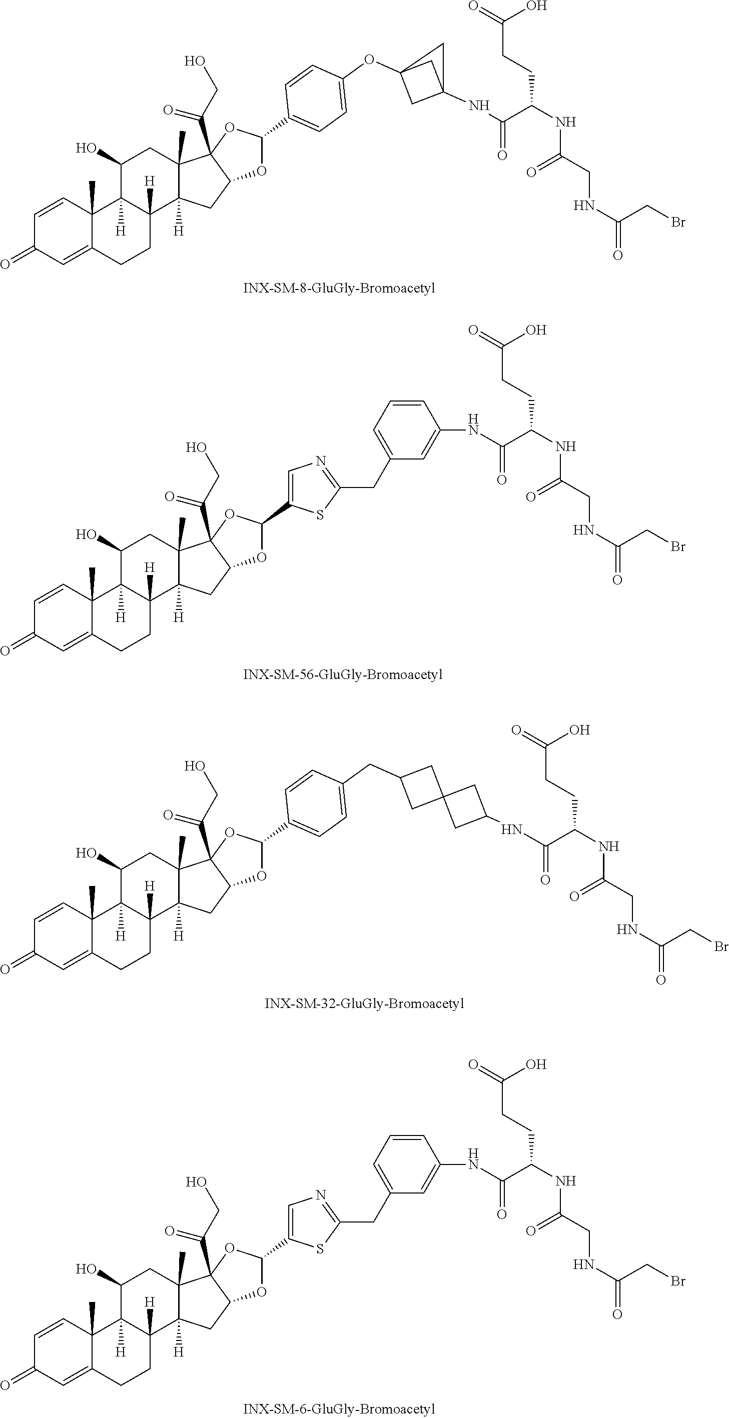

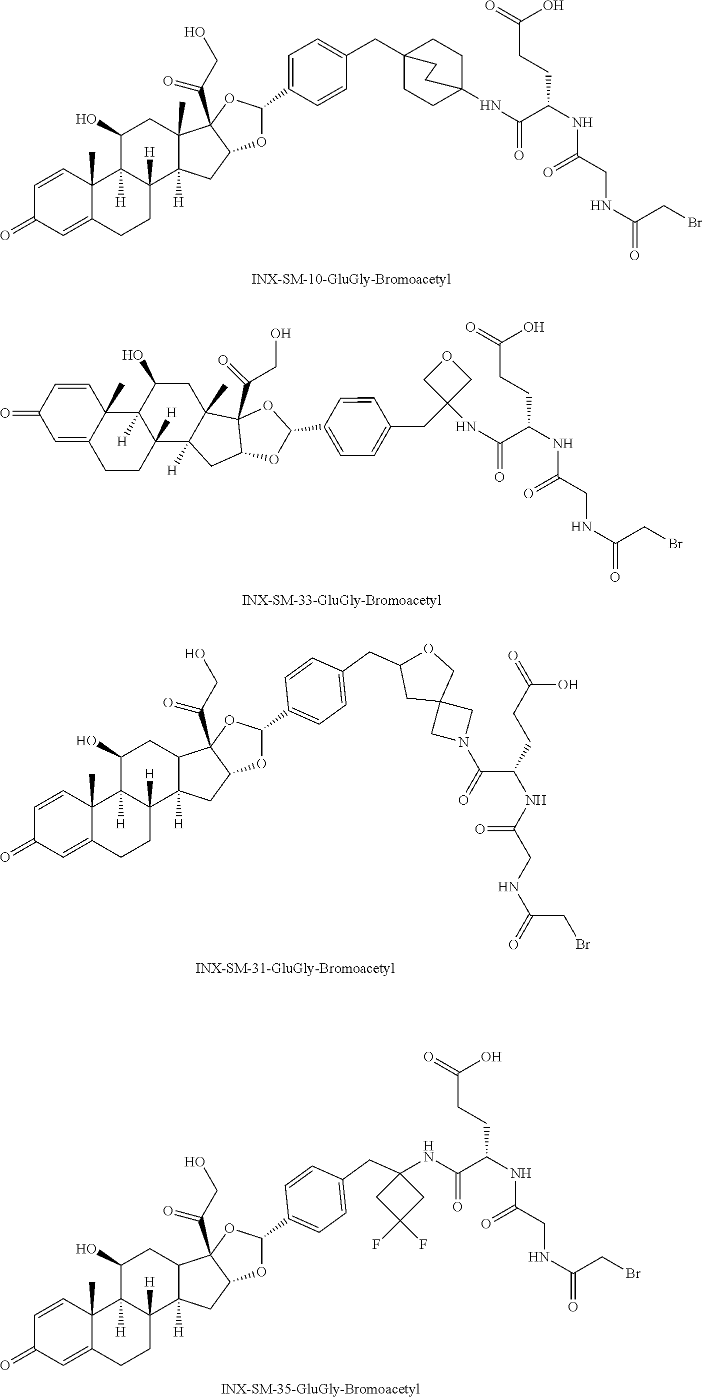

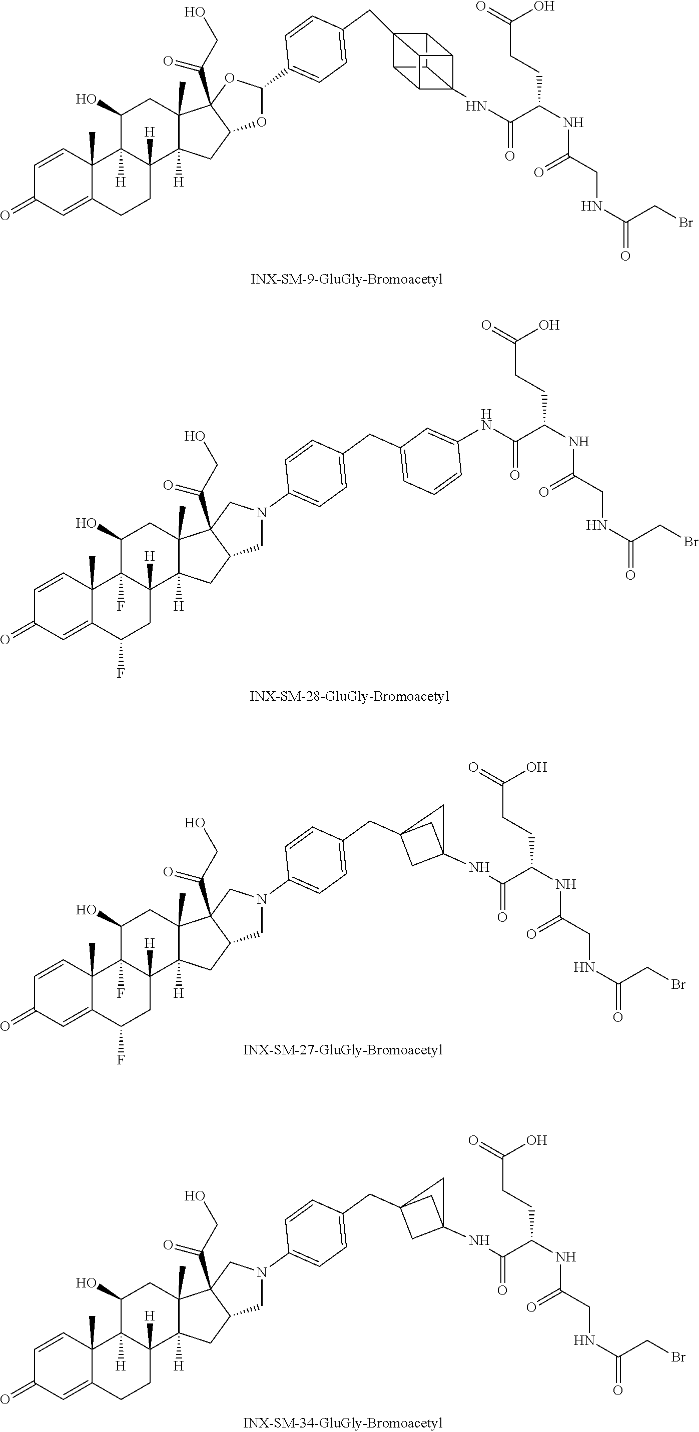

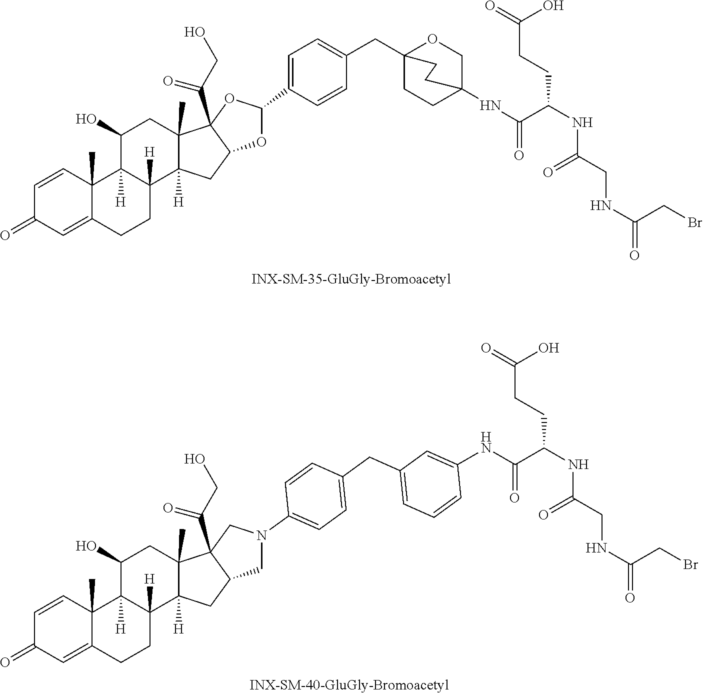

Where,
J=Dibenzylcyclooctyne
Linker=Protease cleavable sequence (AA)

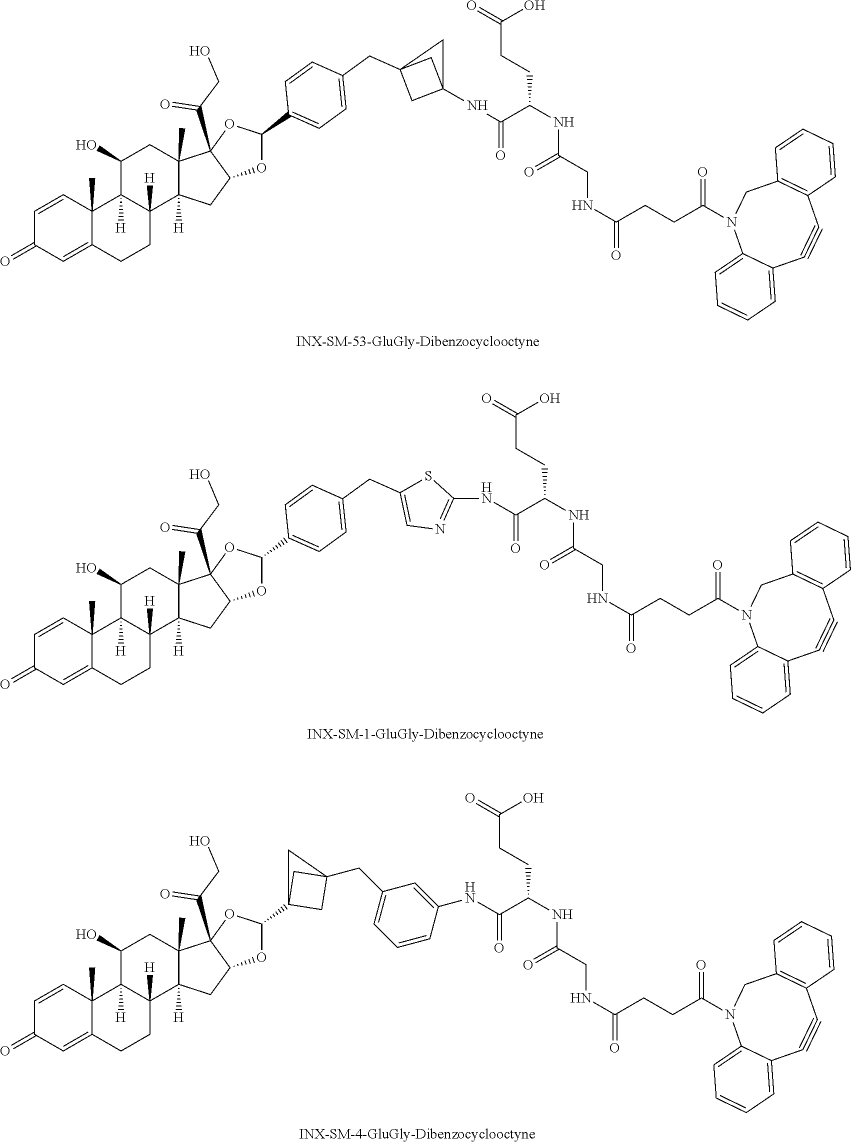

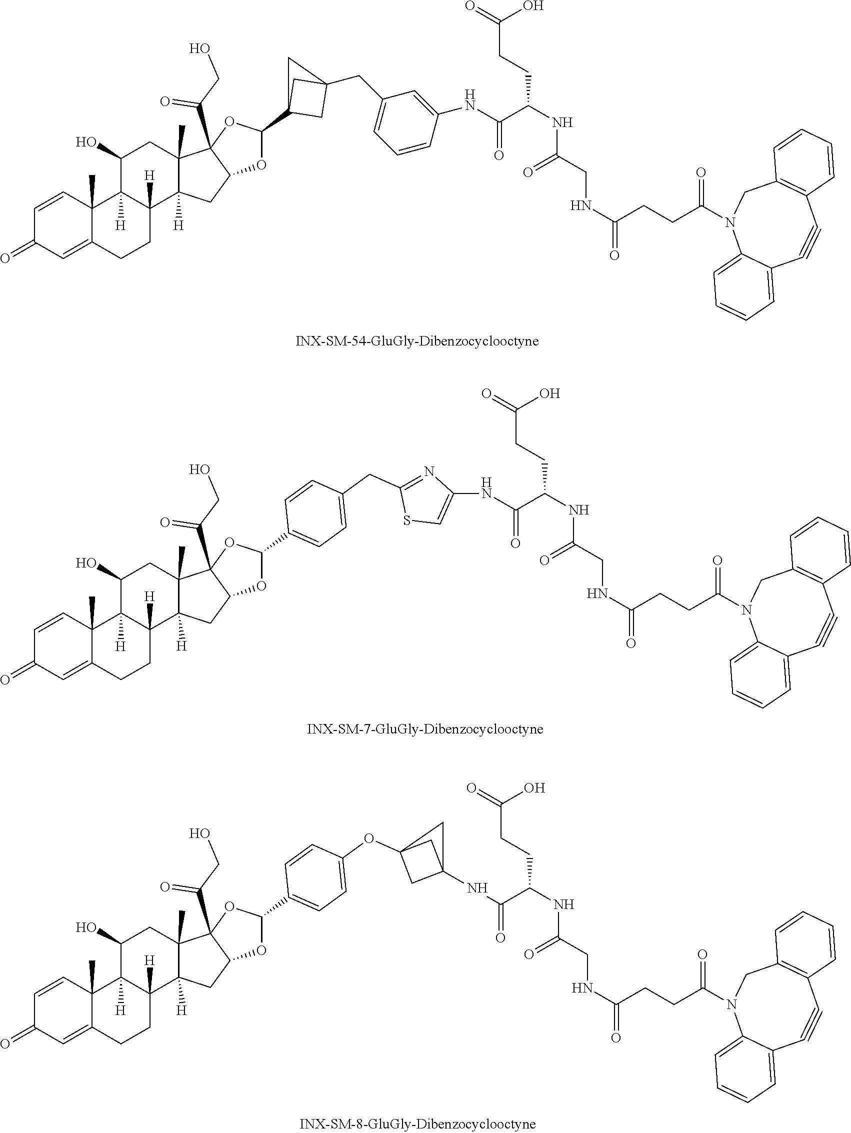

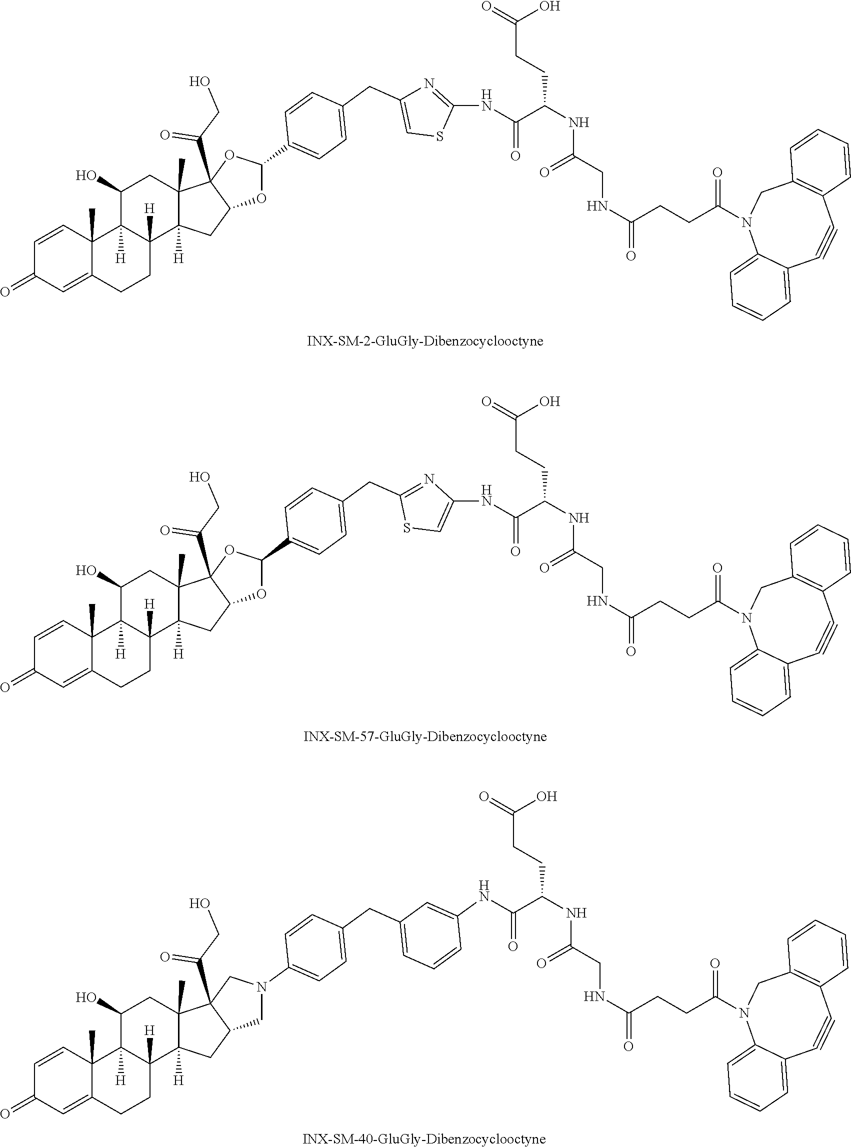

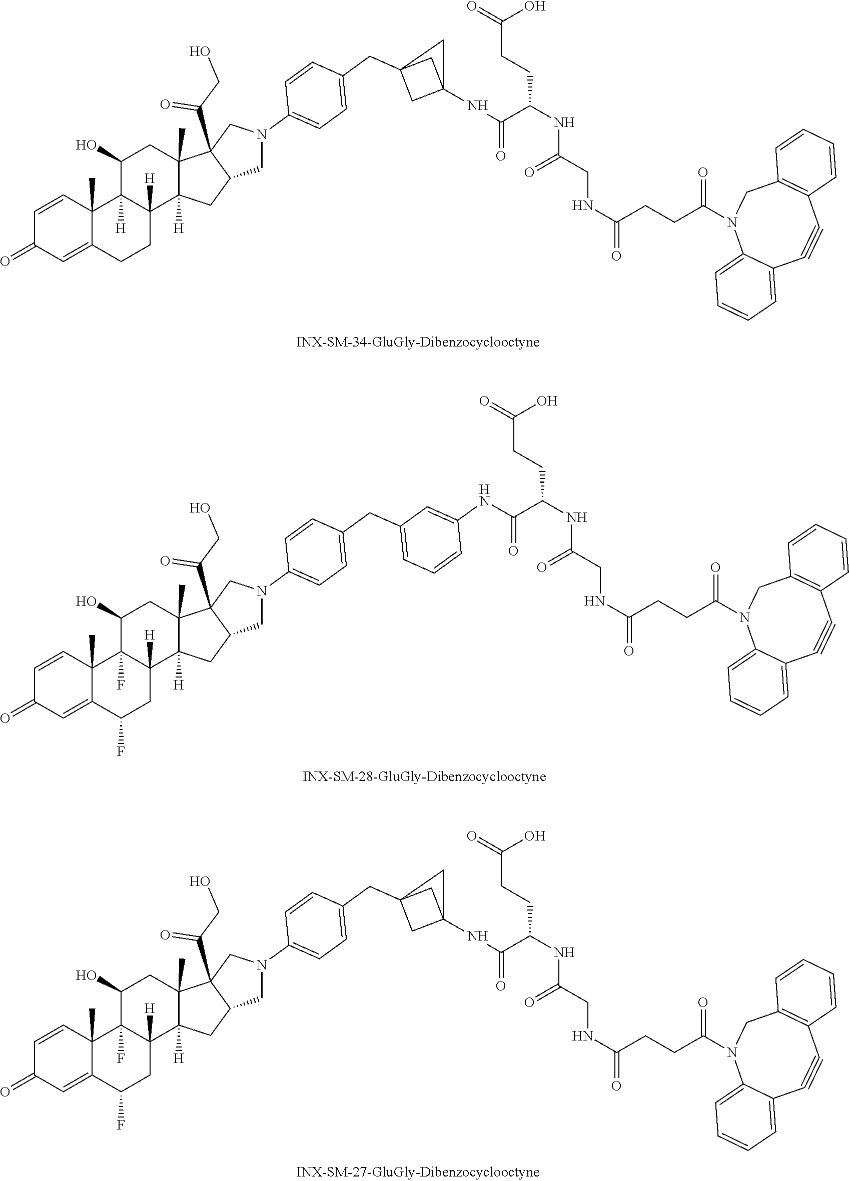

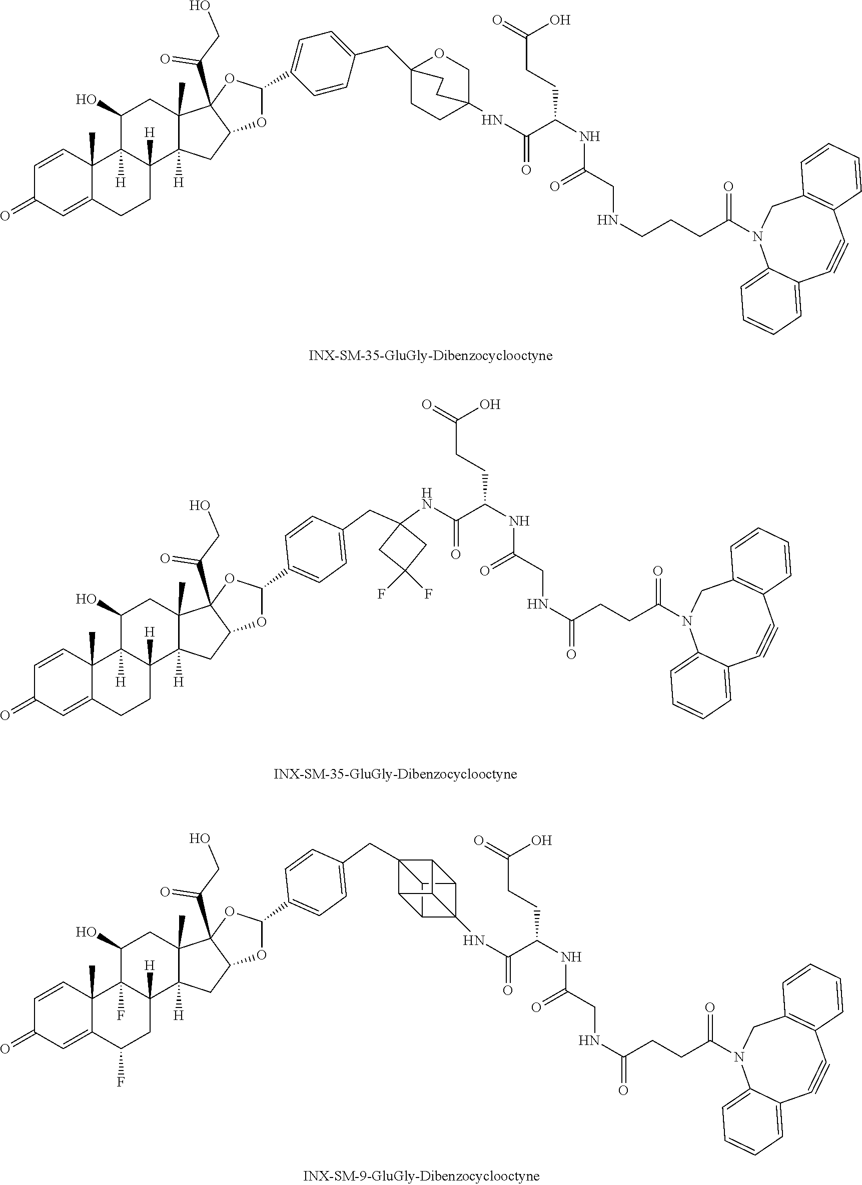

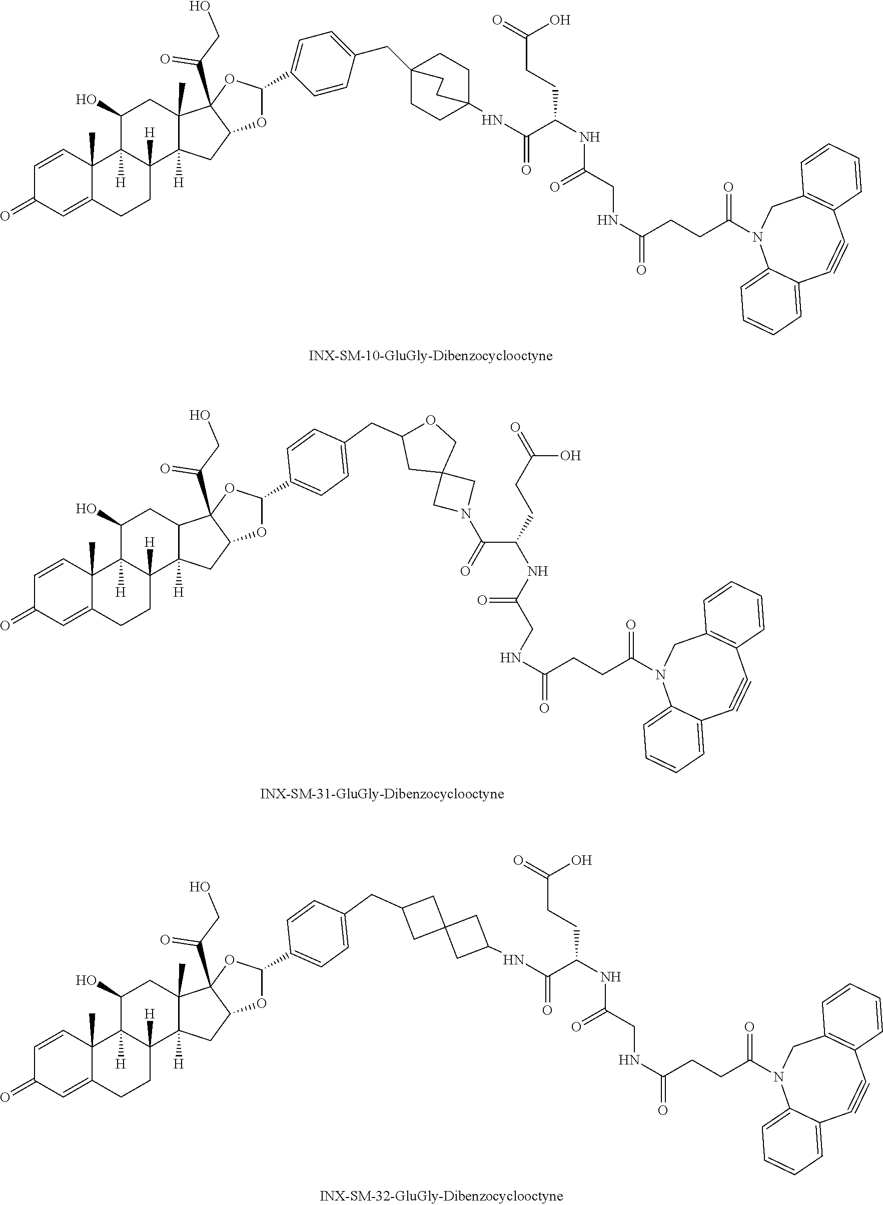

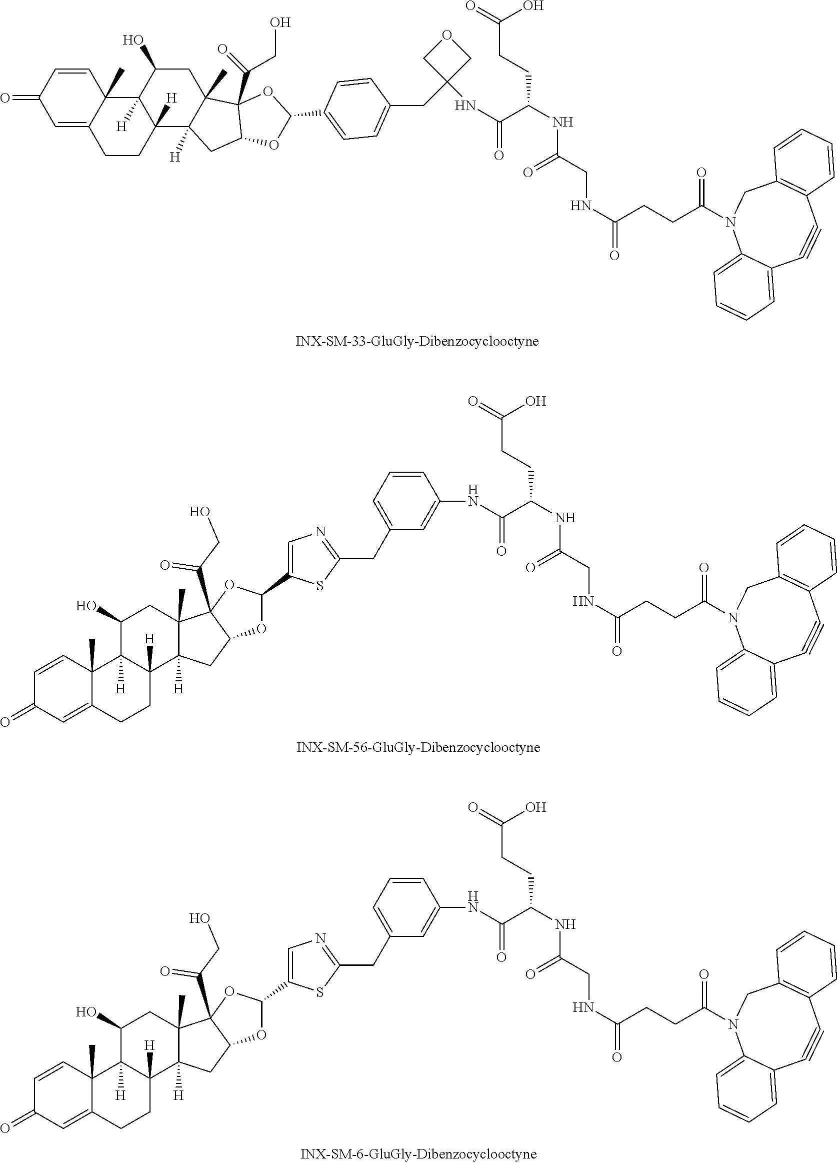

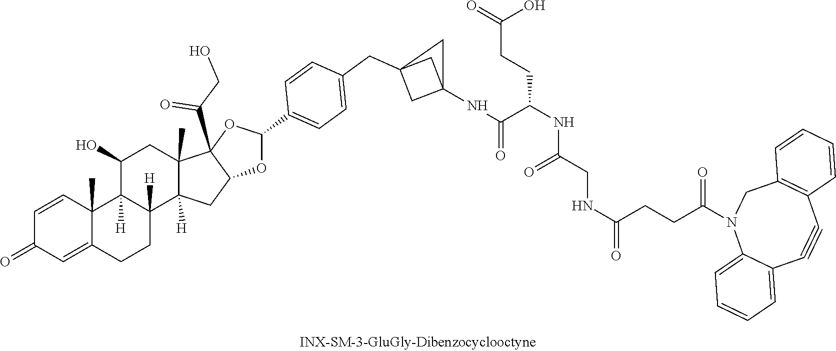

Where,
J=Hydroxysuccinimide
J=Hydroxysuccinimide

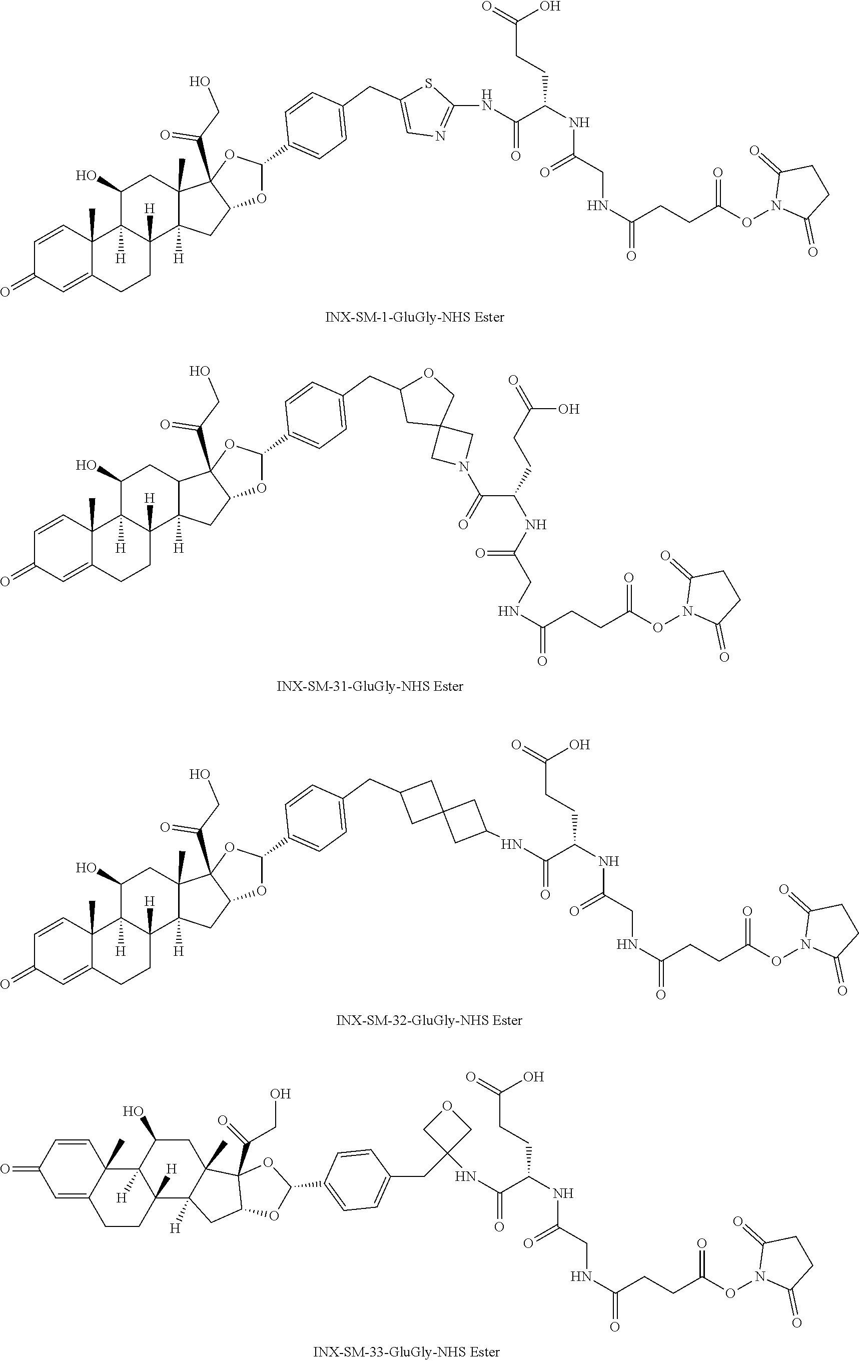

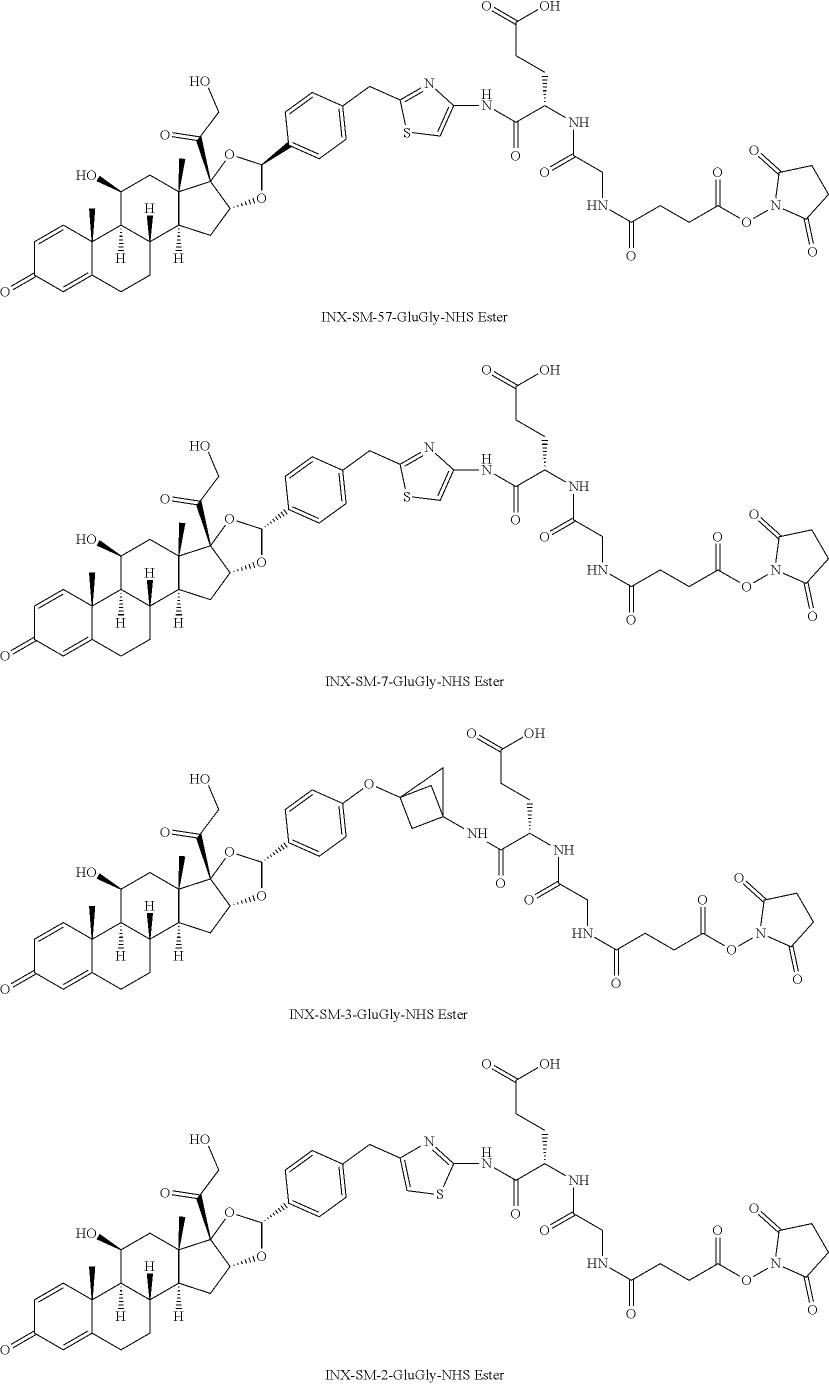

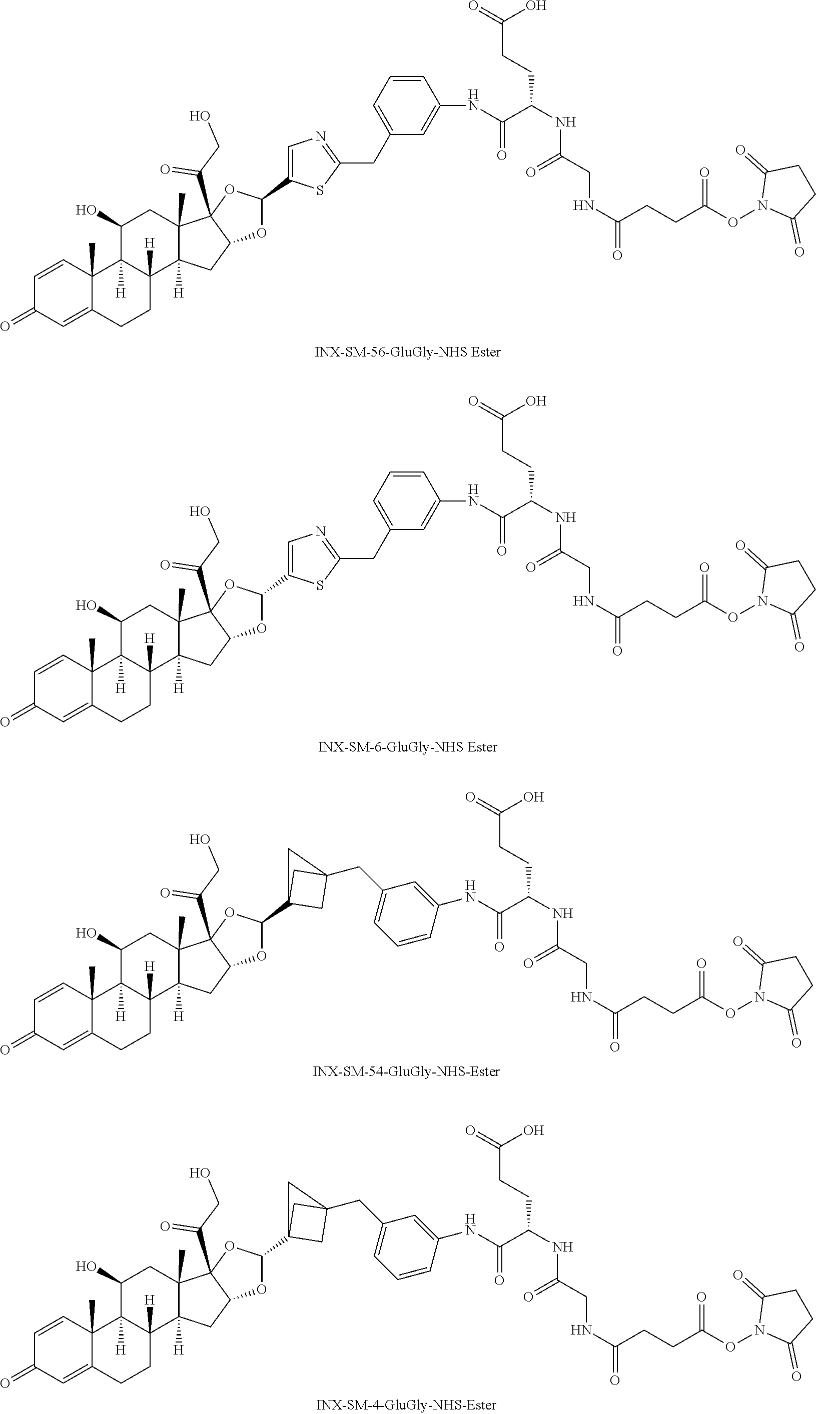

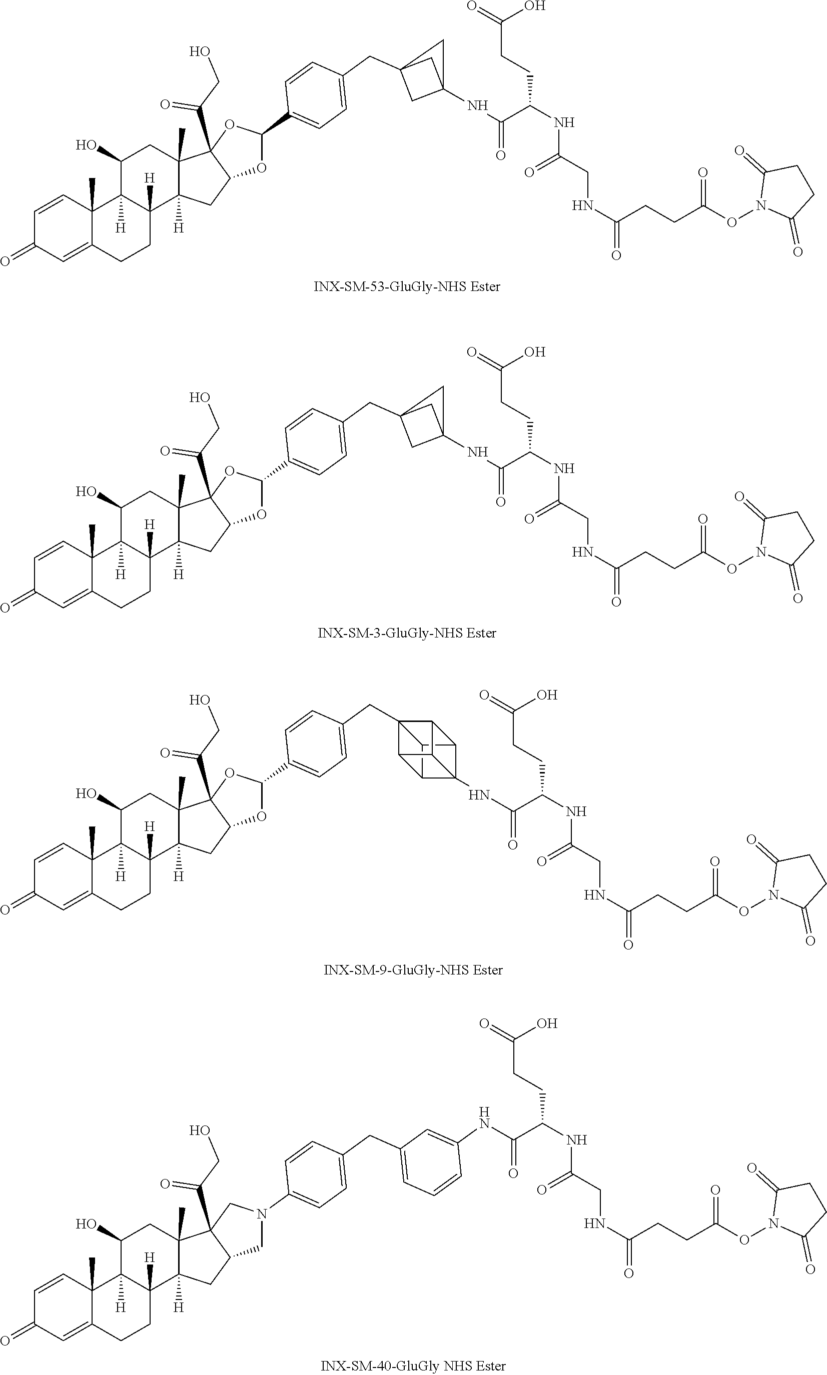

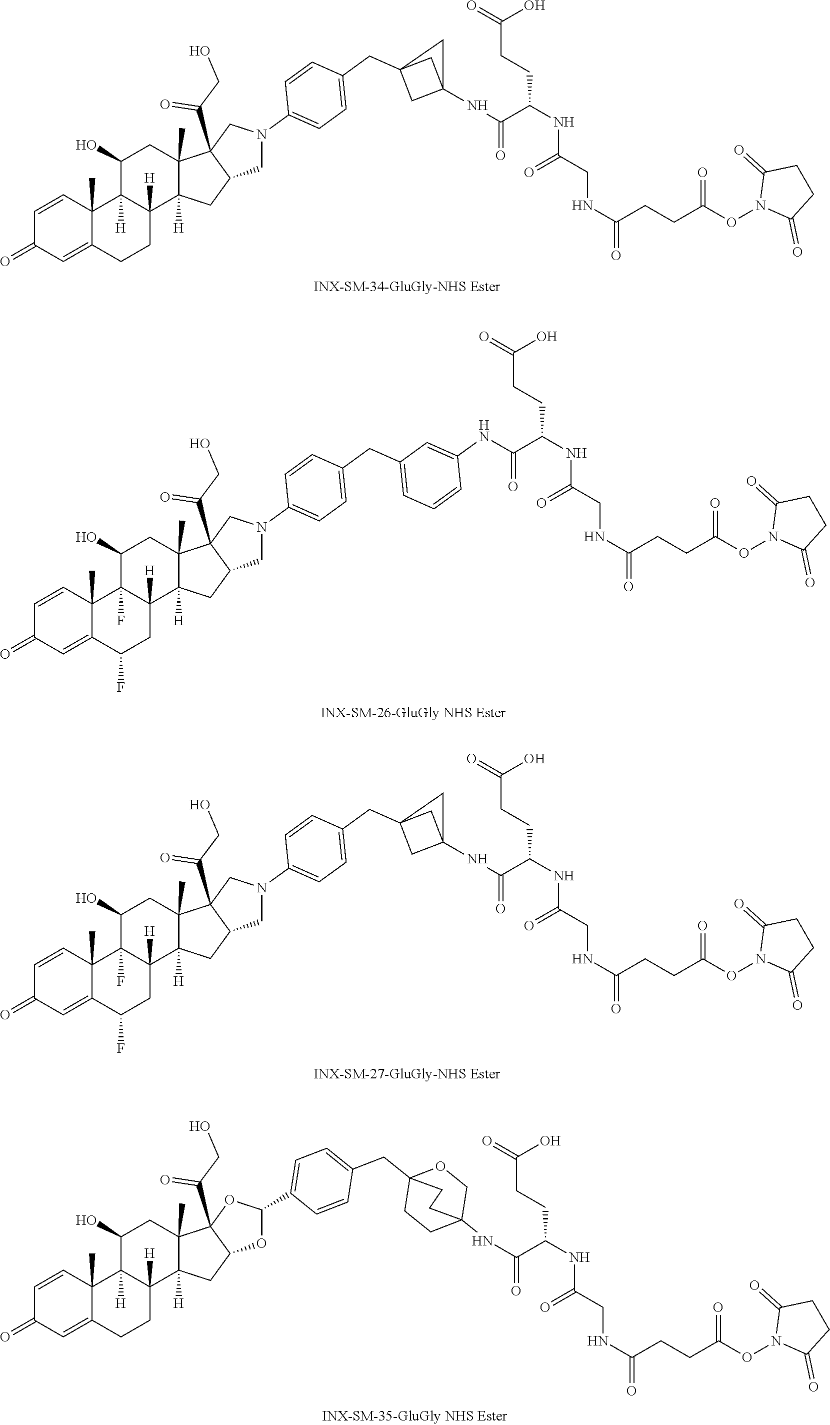

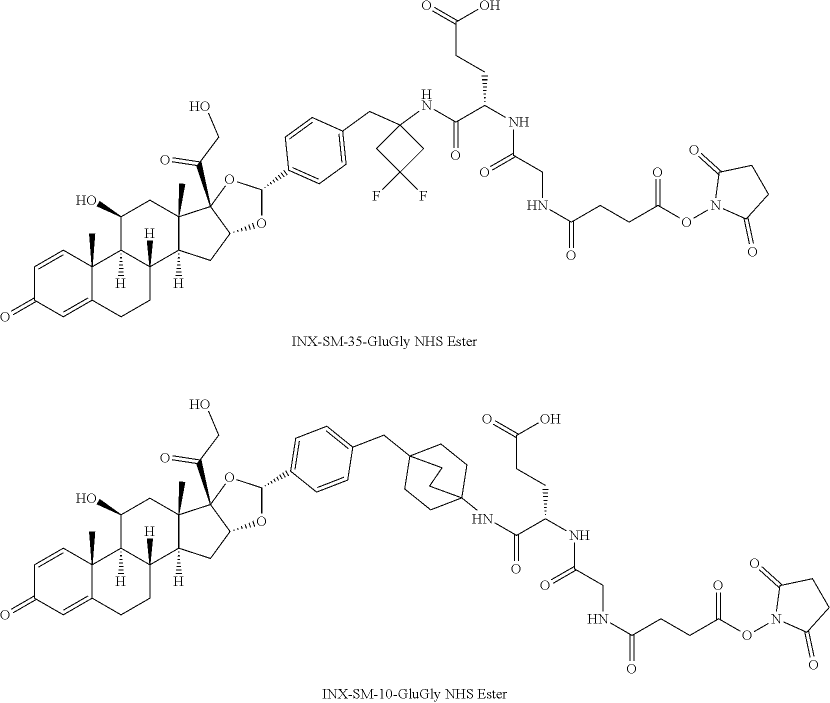

Where,
Linker=Protease cleavable sequence (AA)
J=Maleimide

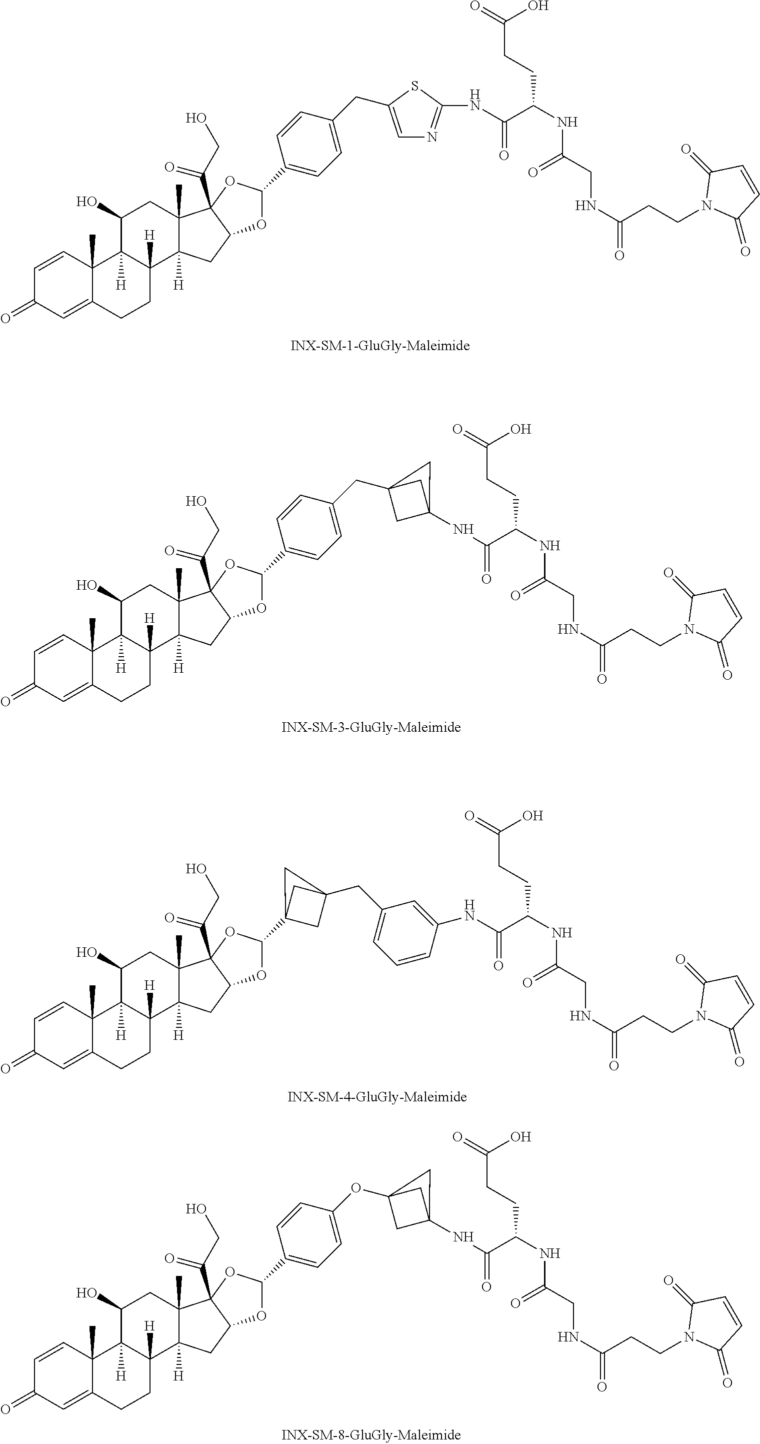

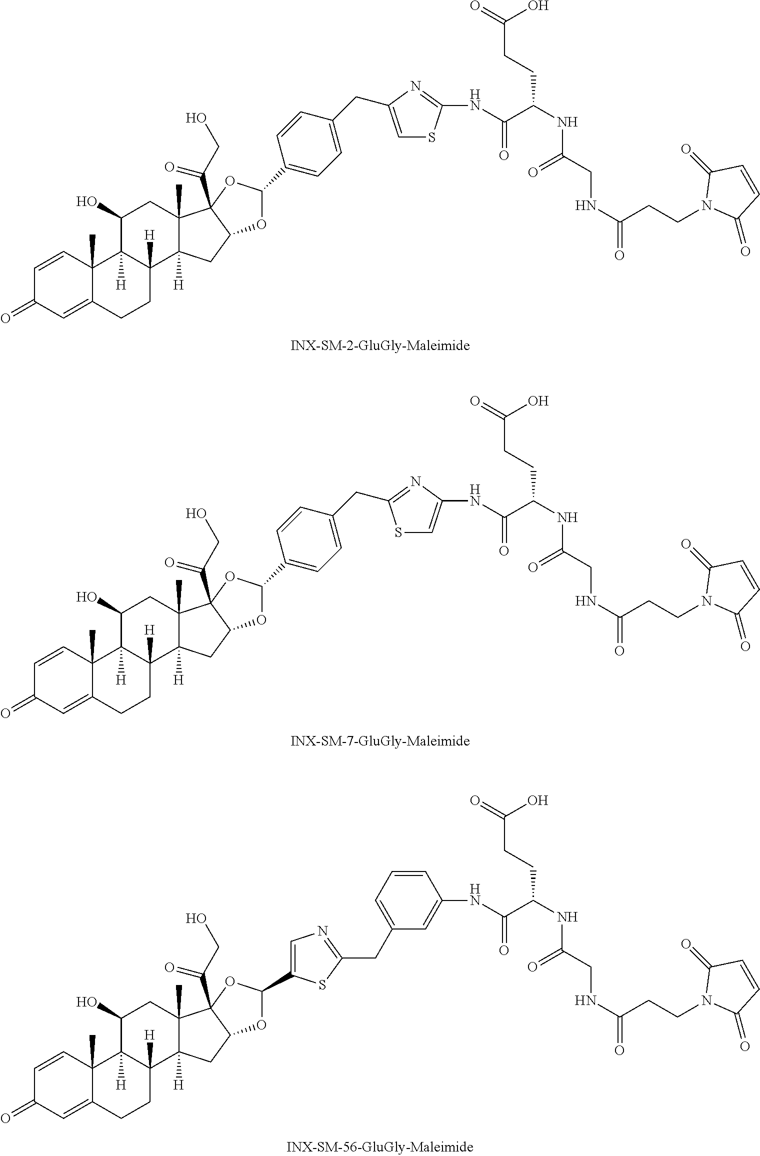

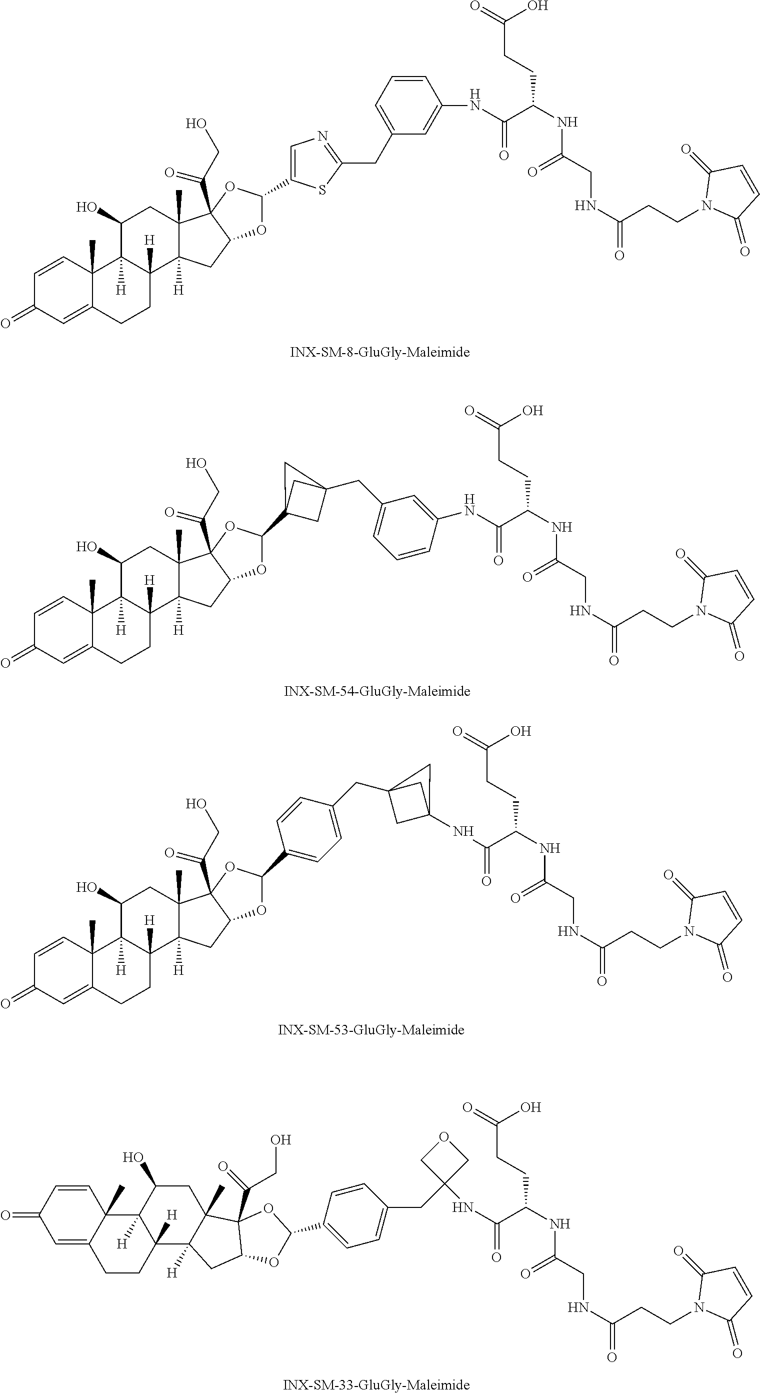

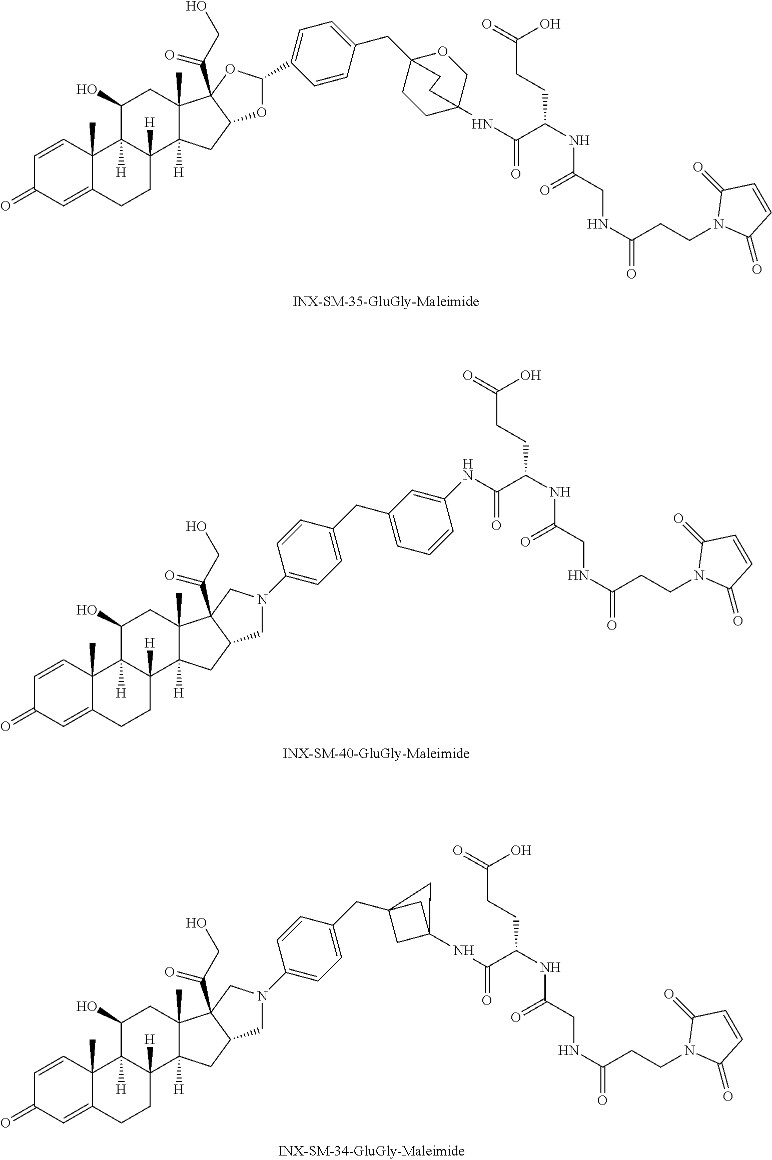

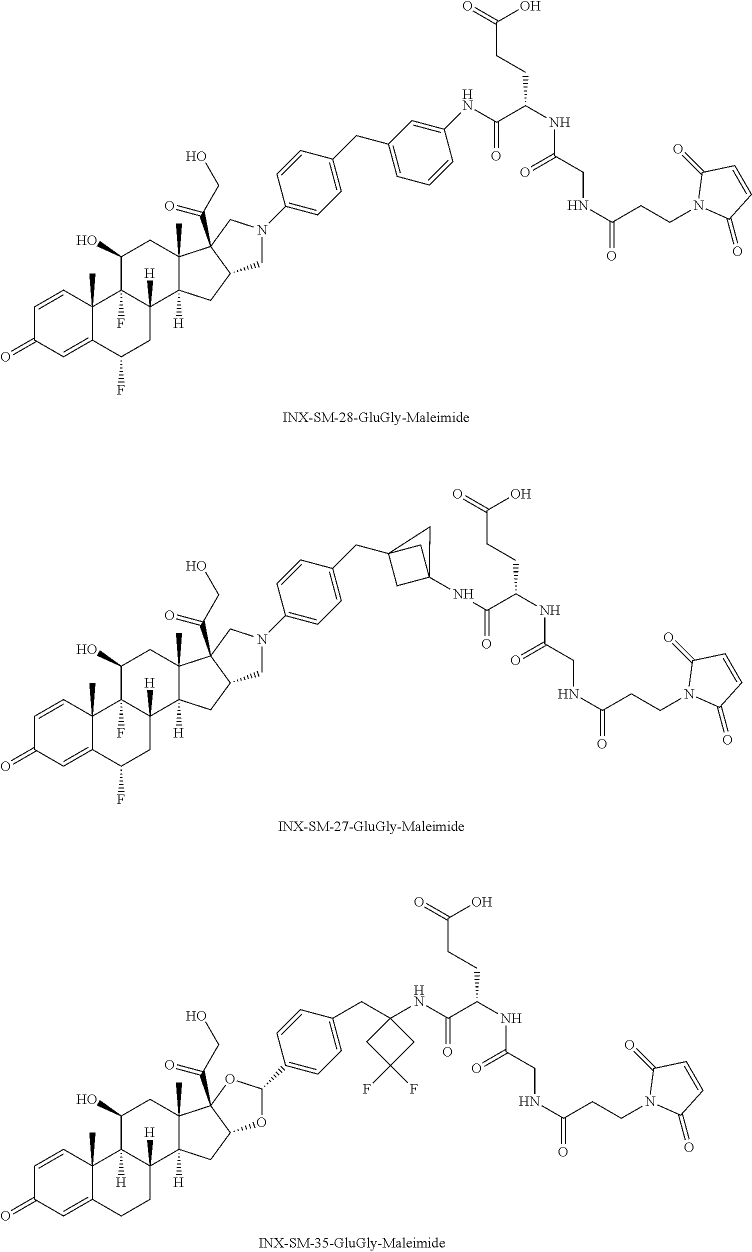

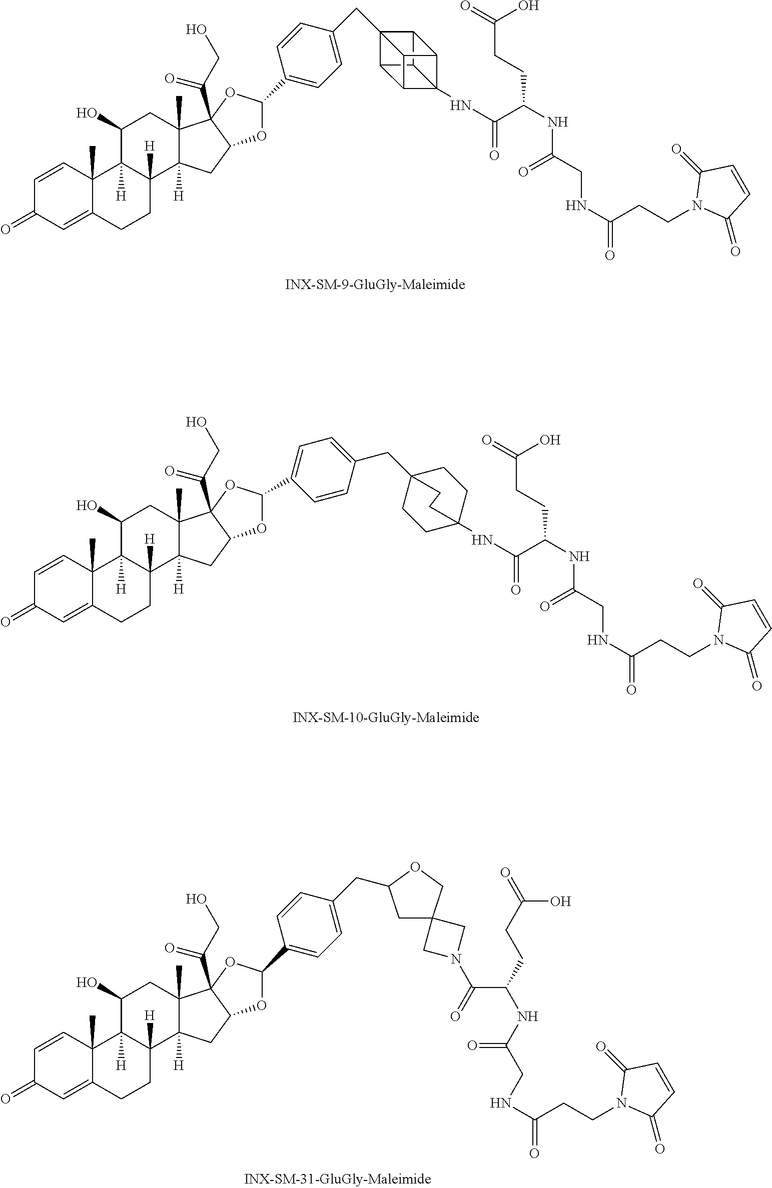

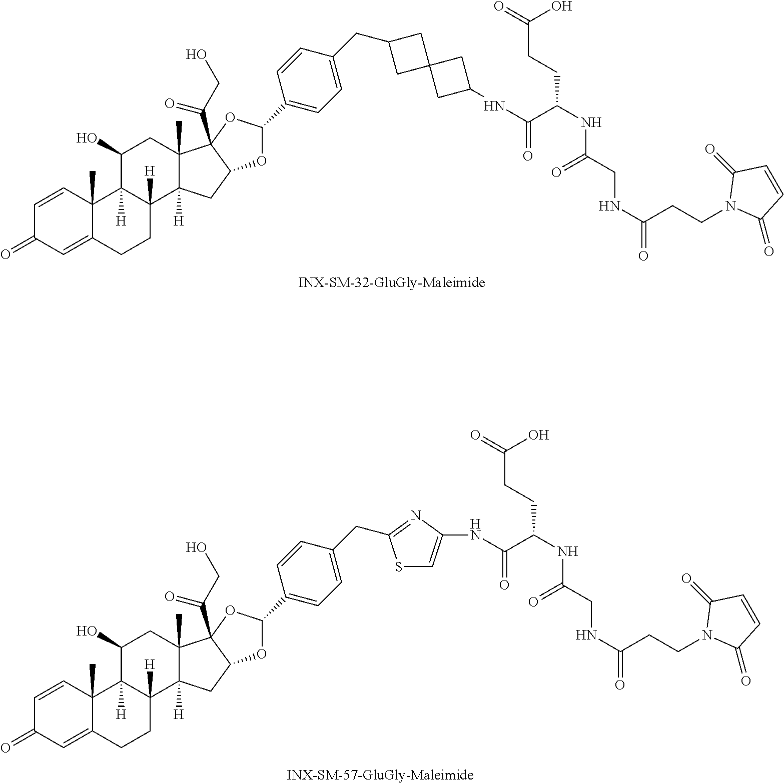

Where,
Linker=Protease cleavable sequence (AA)
J=Tetrazine

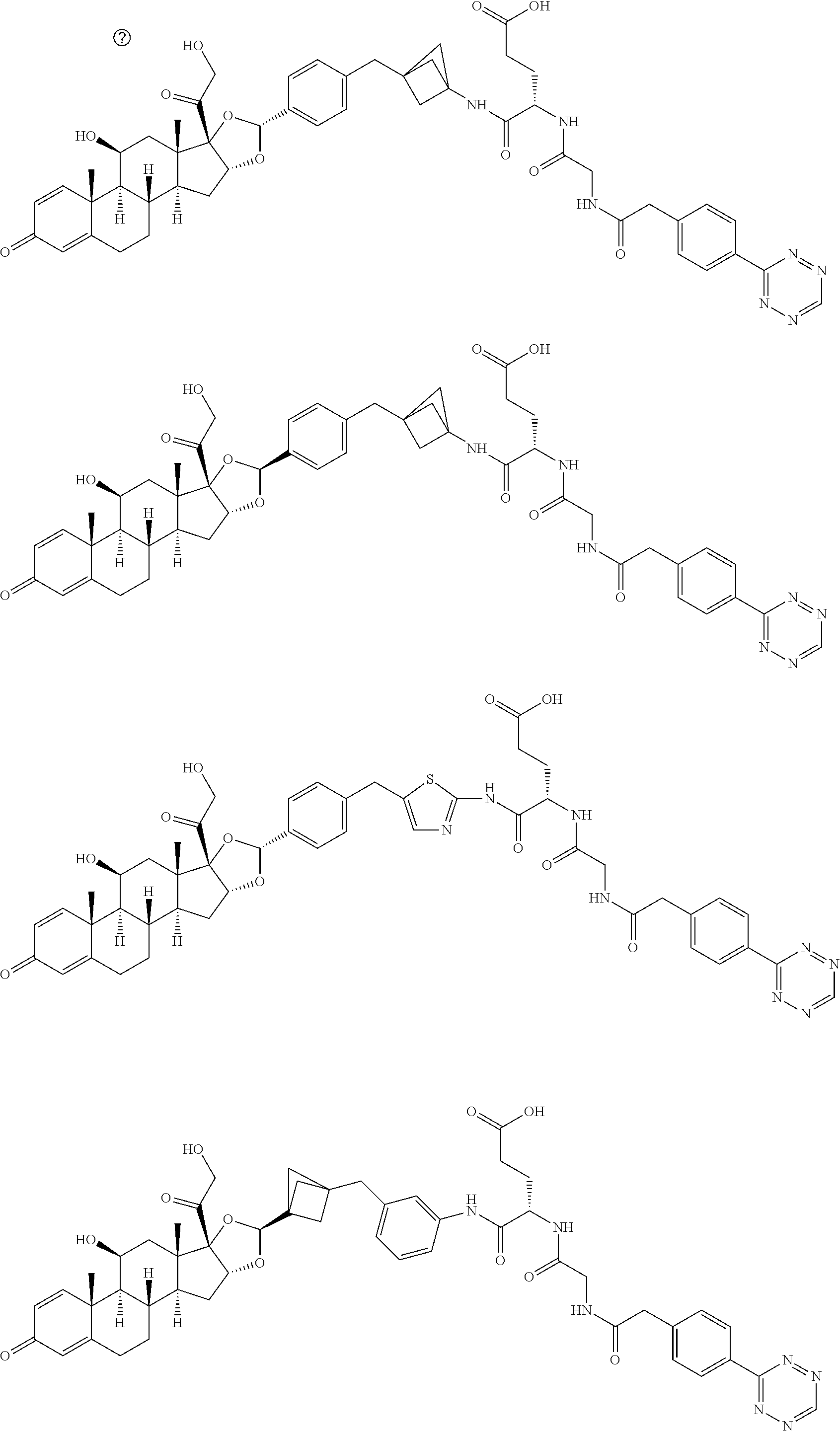

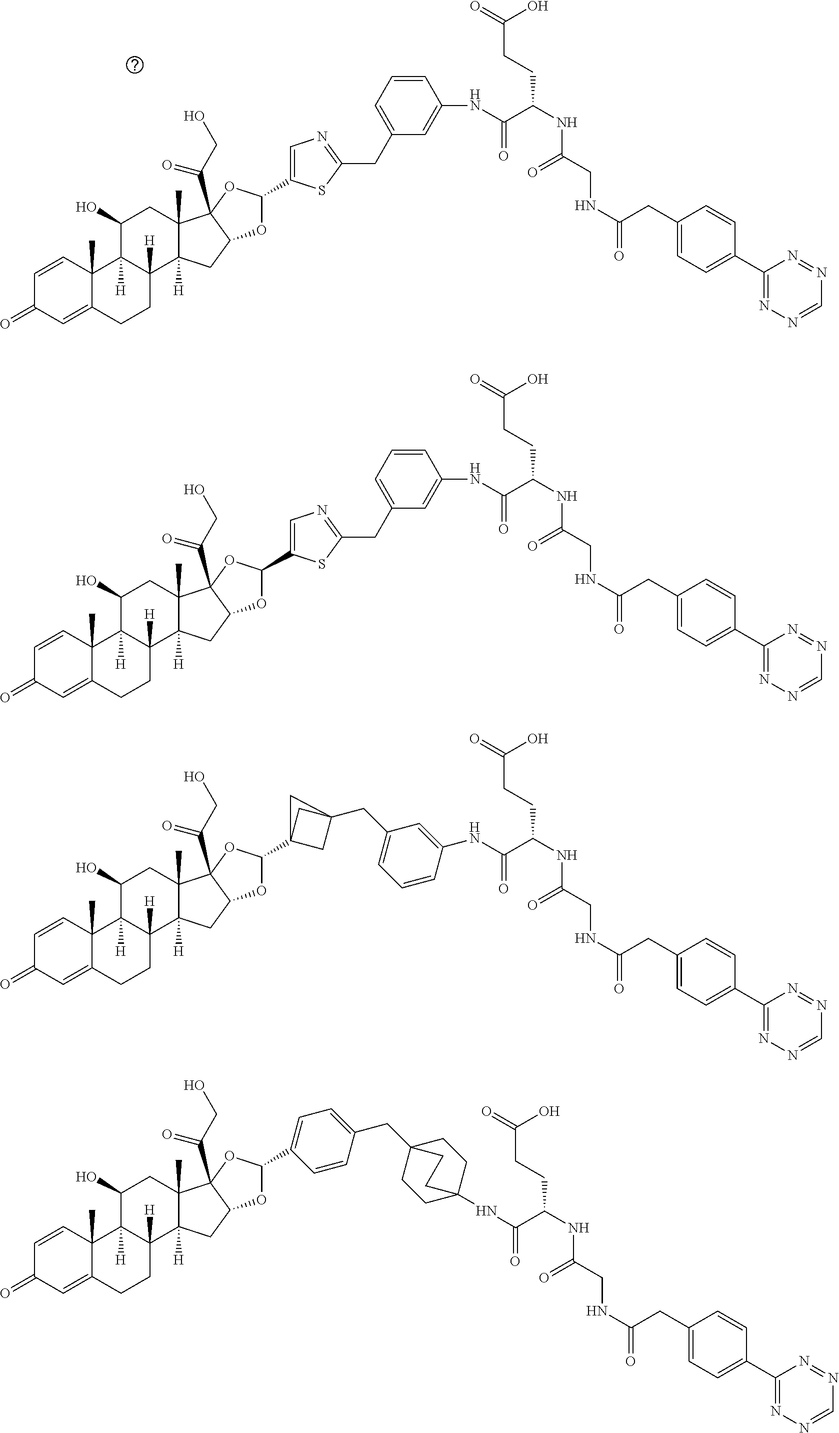

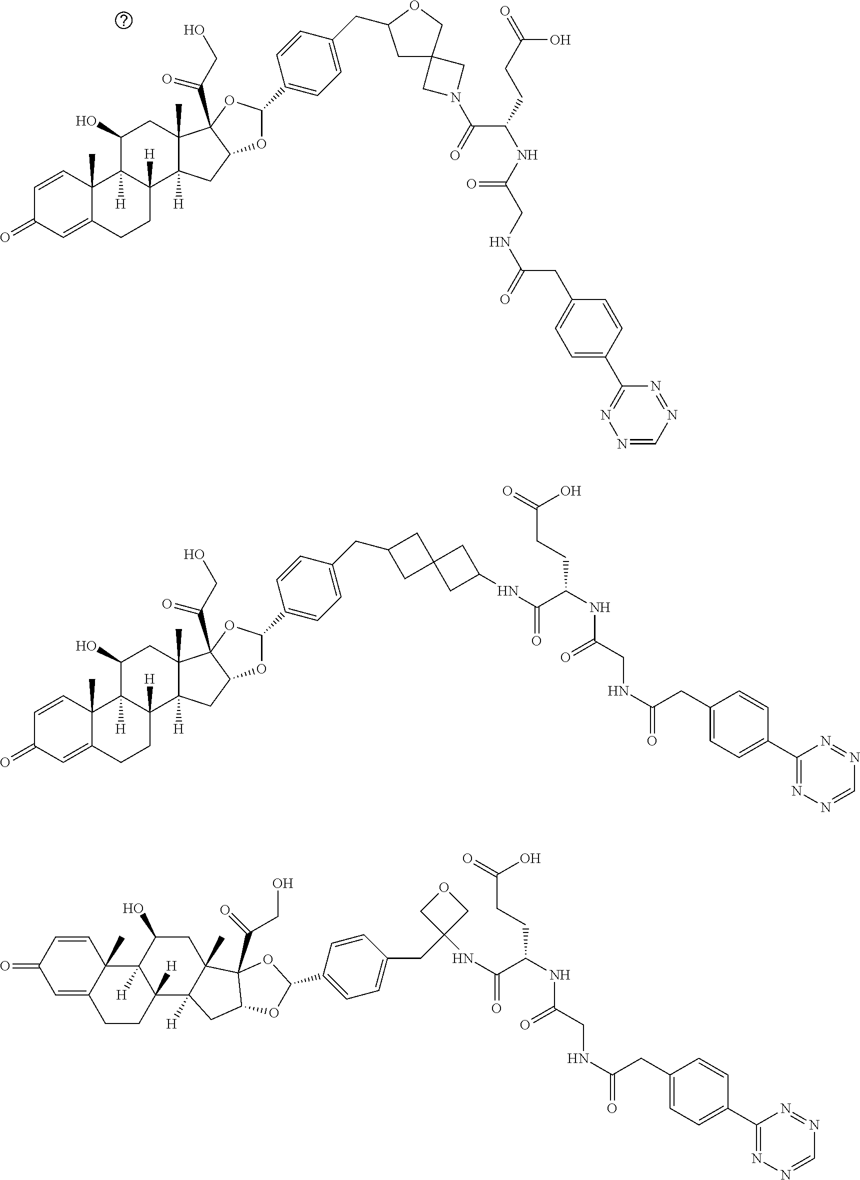

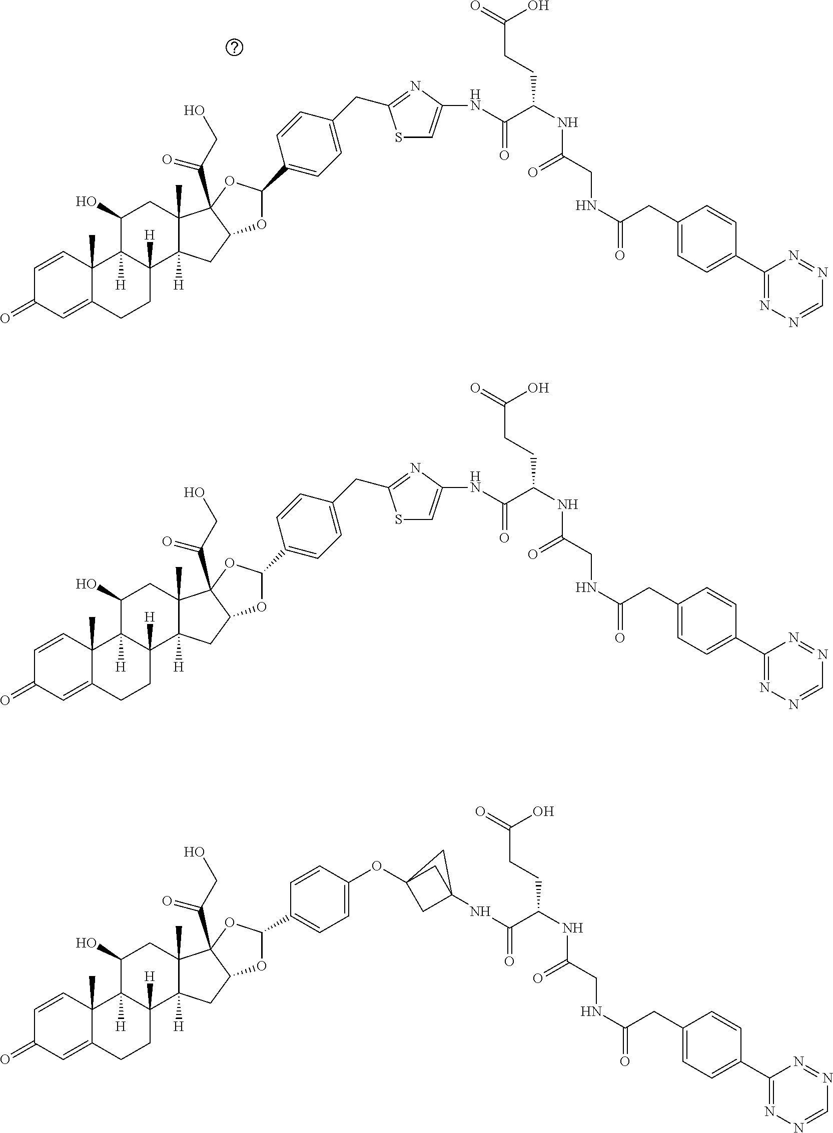

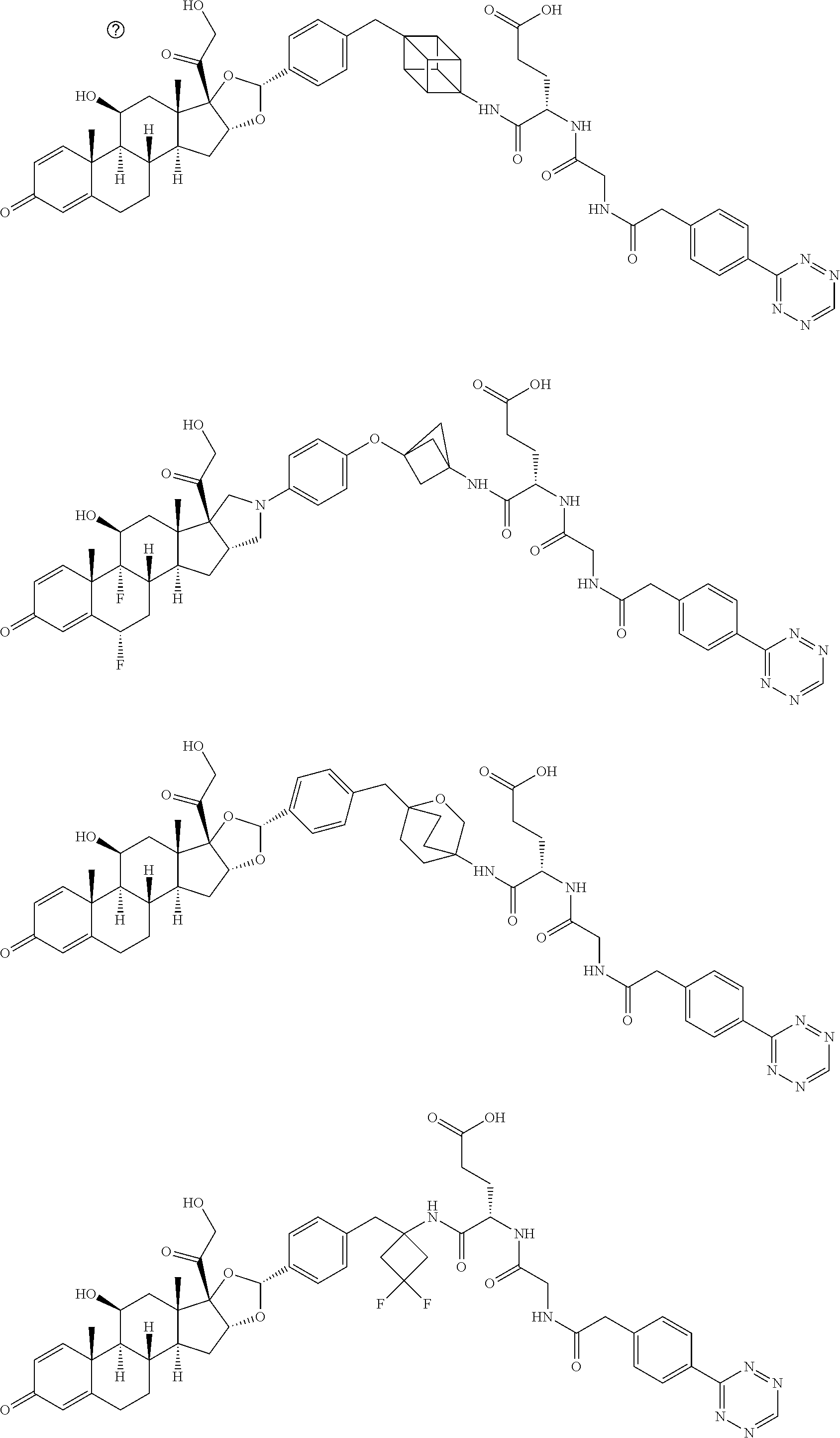

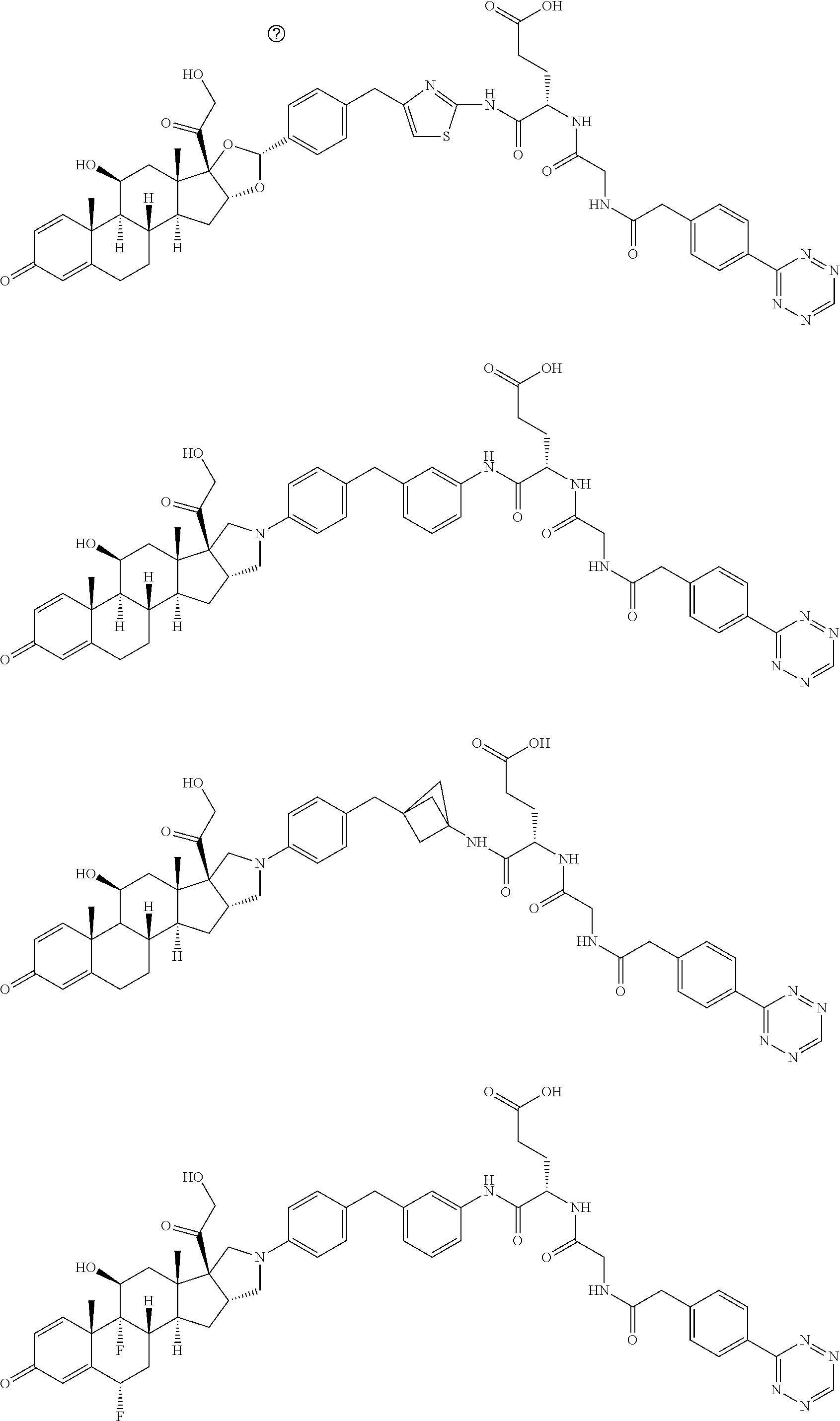

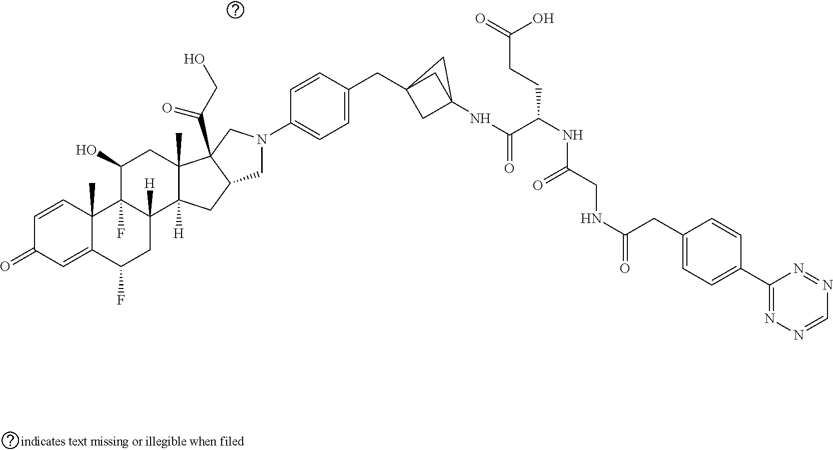

Where,
Linker=Protease cleavable sequence (AA)
J=TG Conjugation

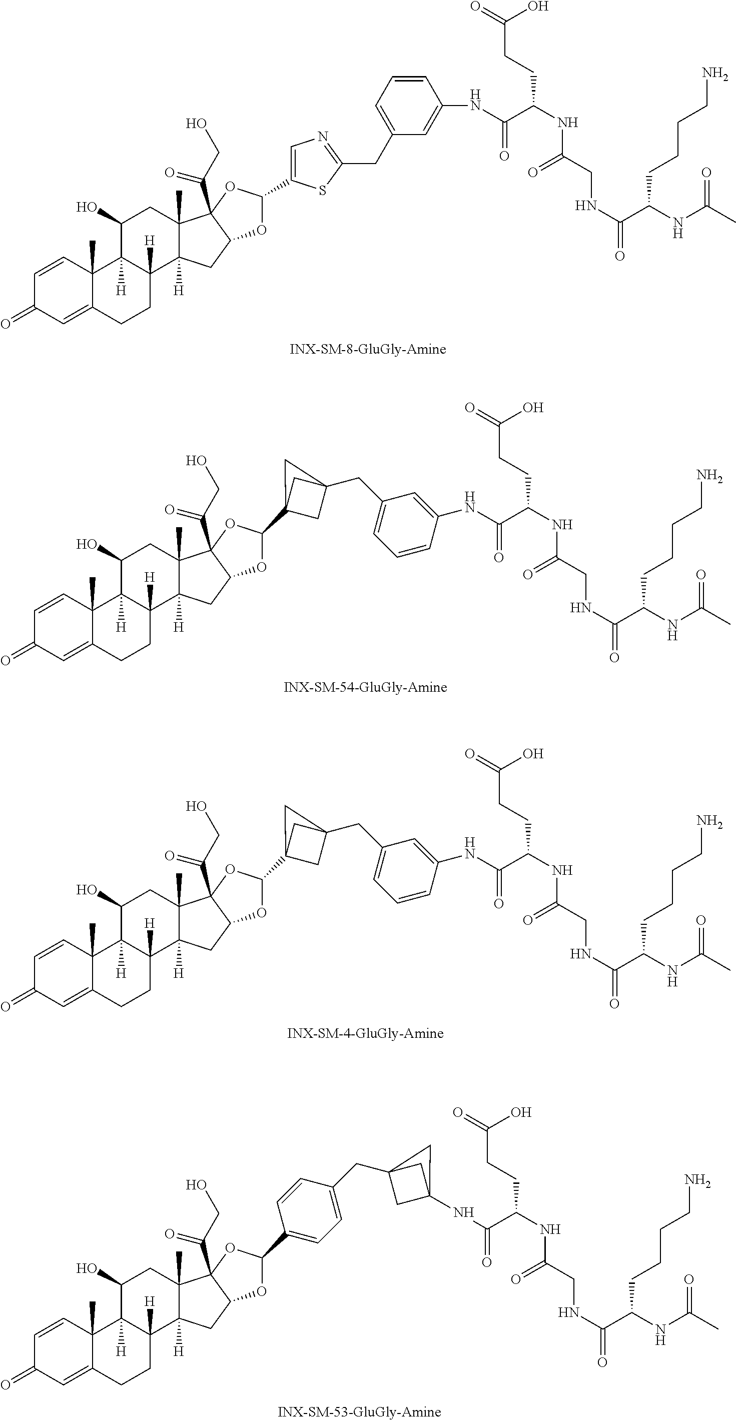

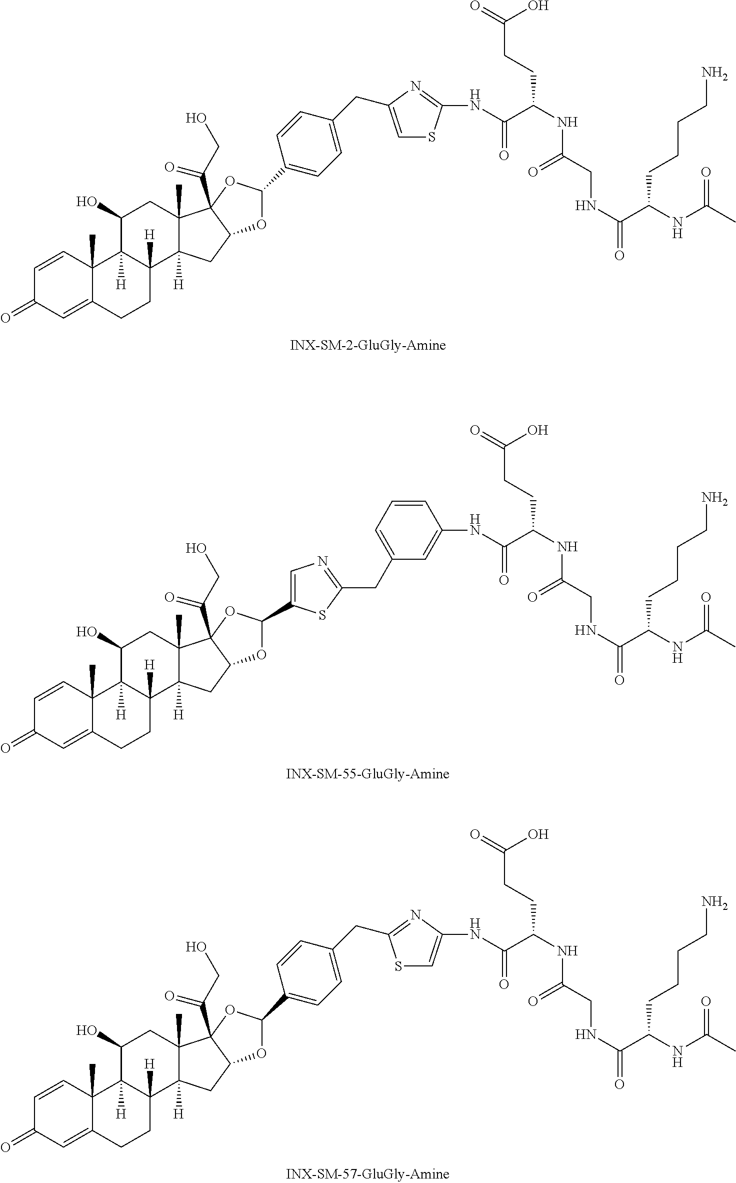

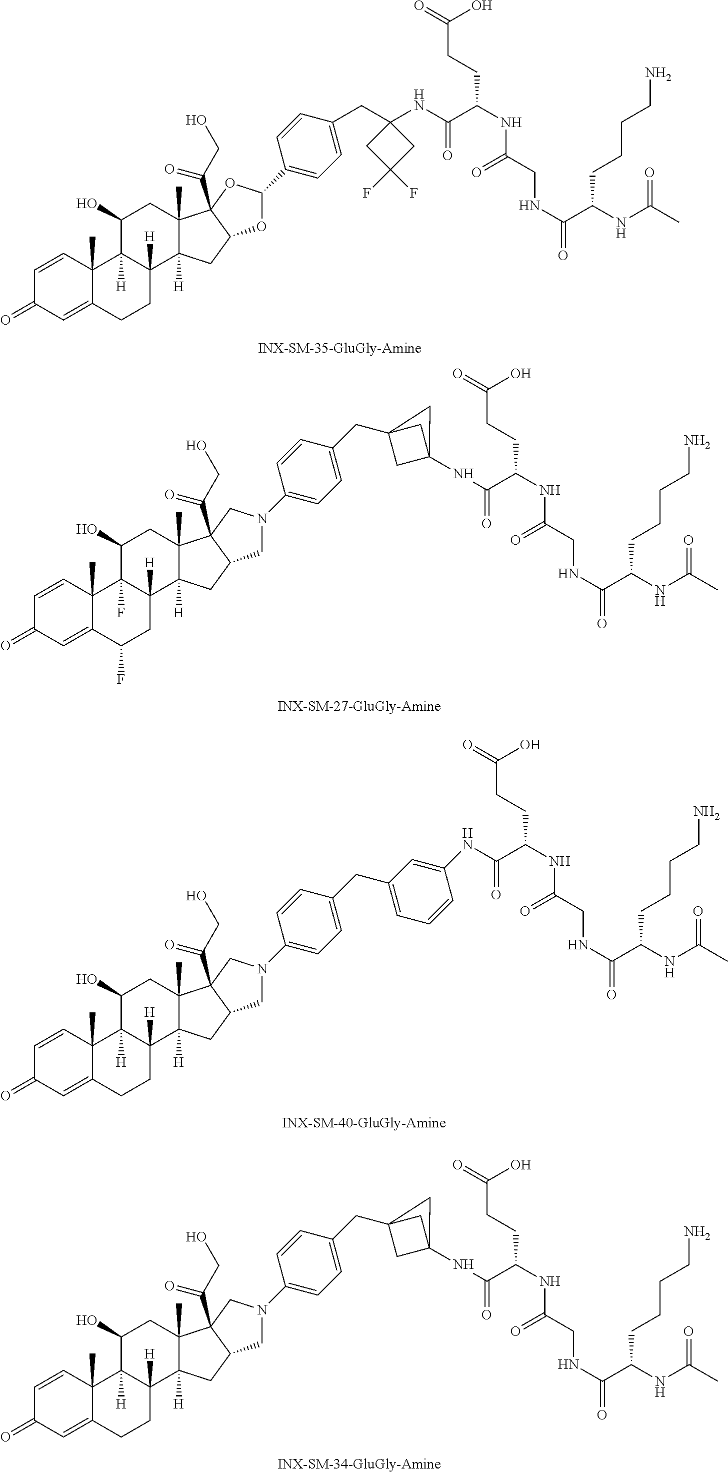

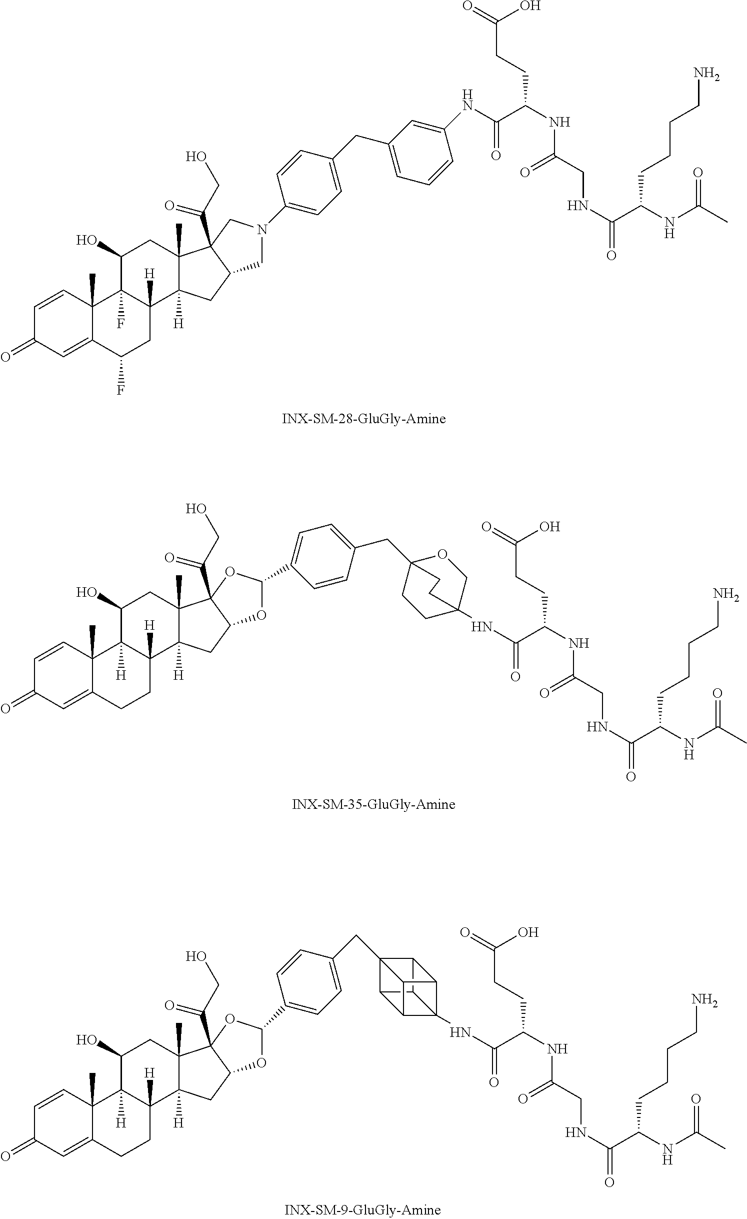

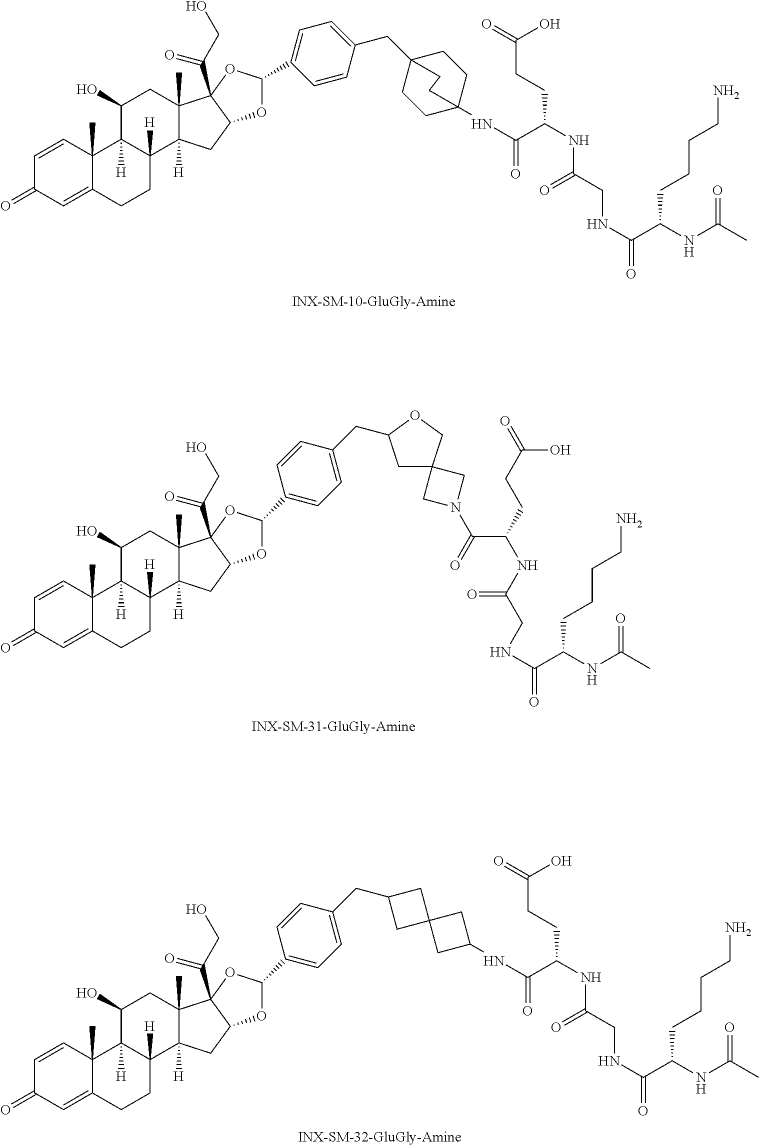

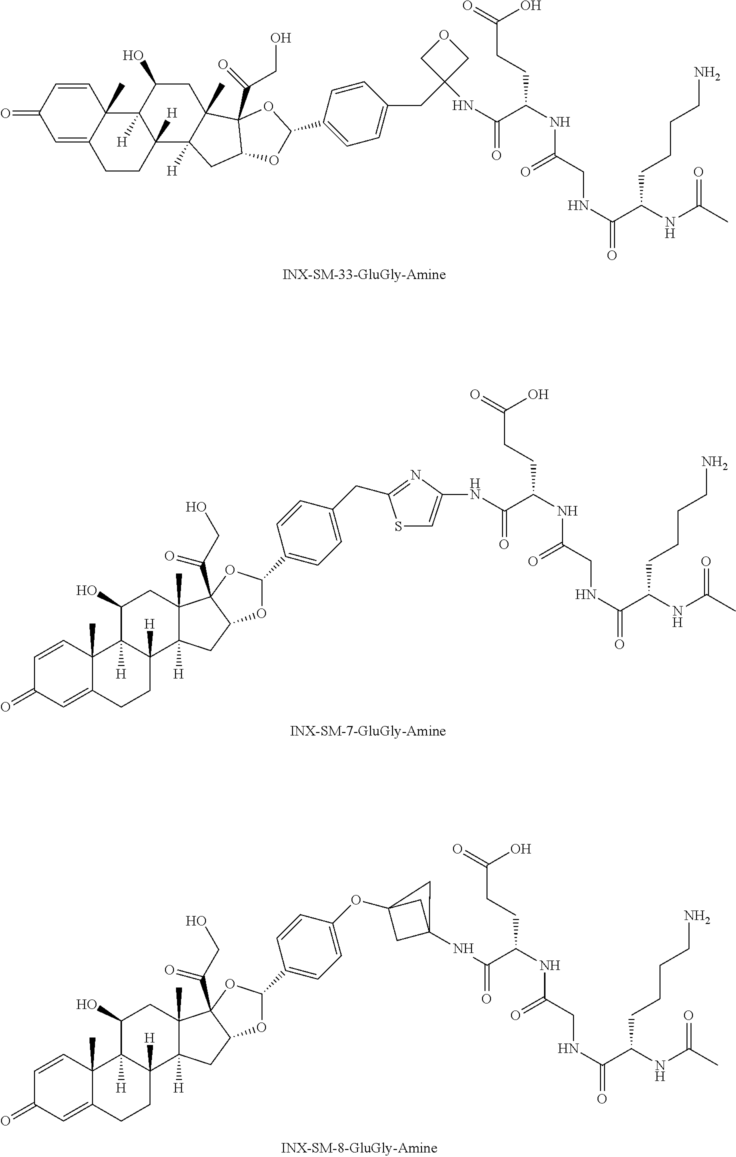

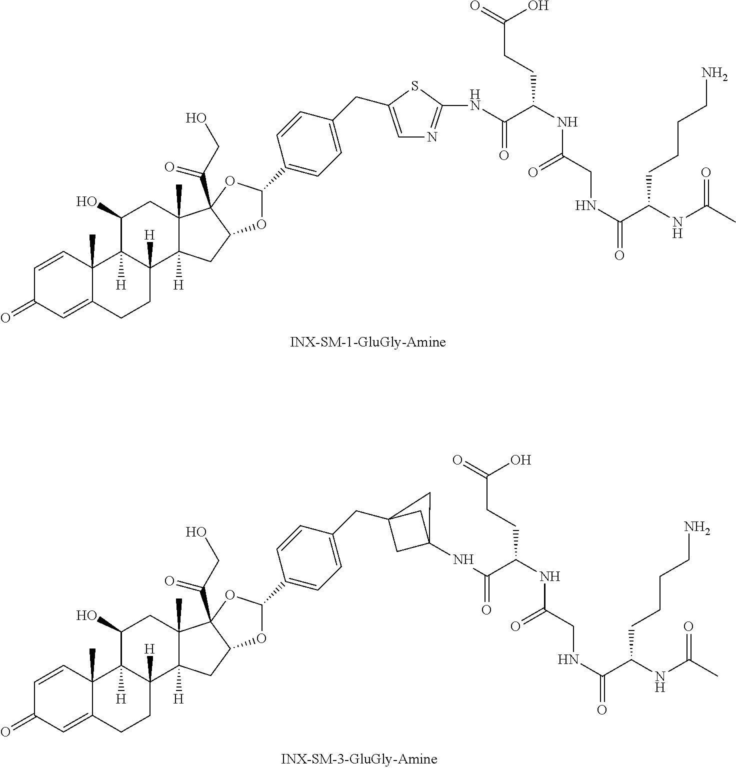

### (II) Payload-Linker-J

Where,
Linker=Protease cleavable sequence (AA)+Immolative linker Para Amino Benzyl (PAB)
J=Alkoxyamine

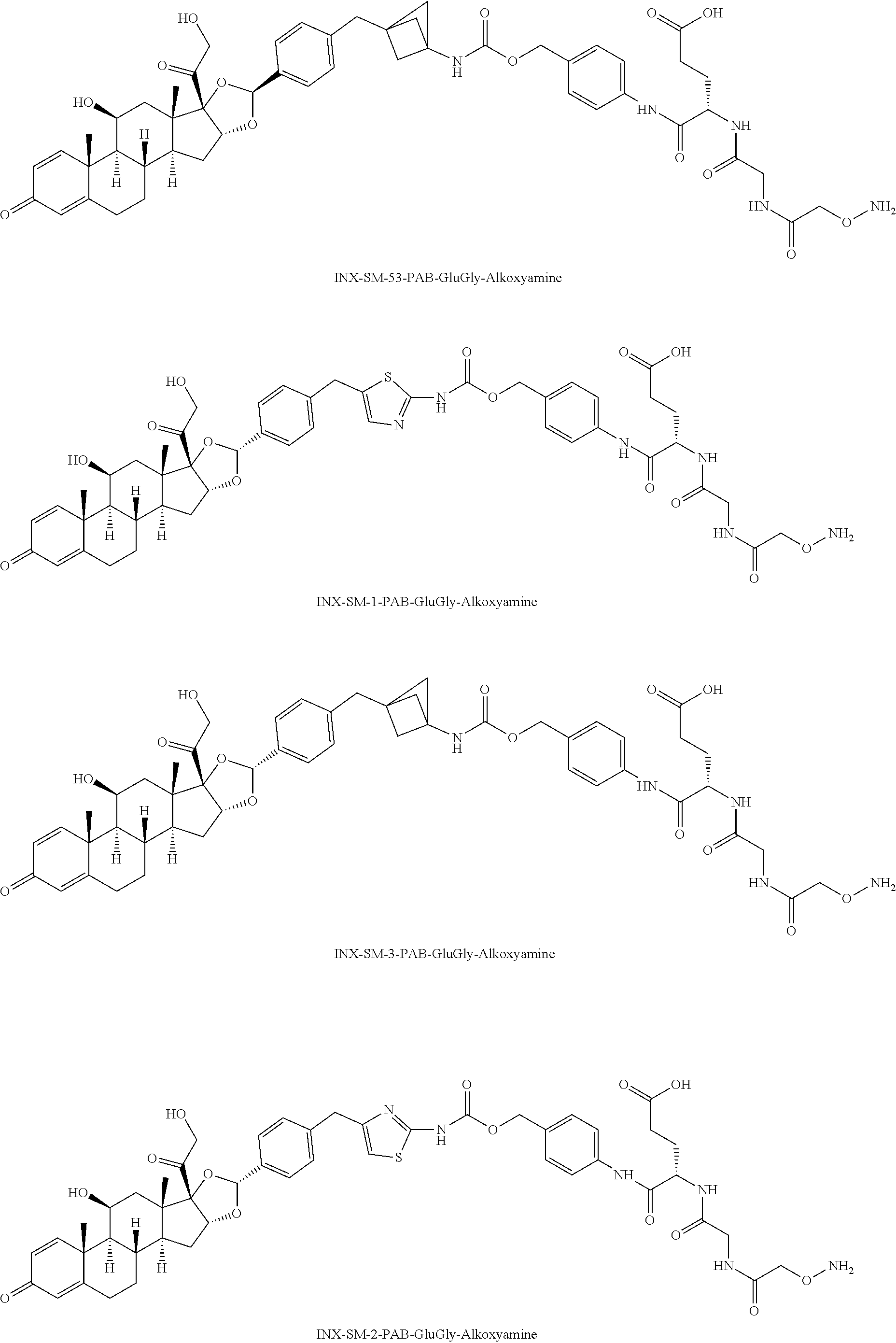

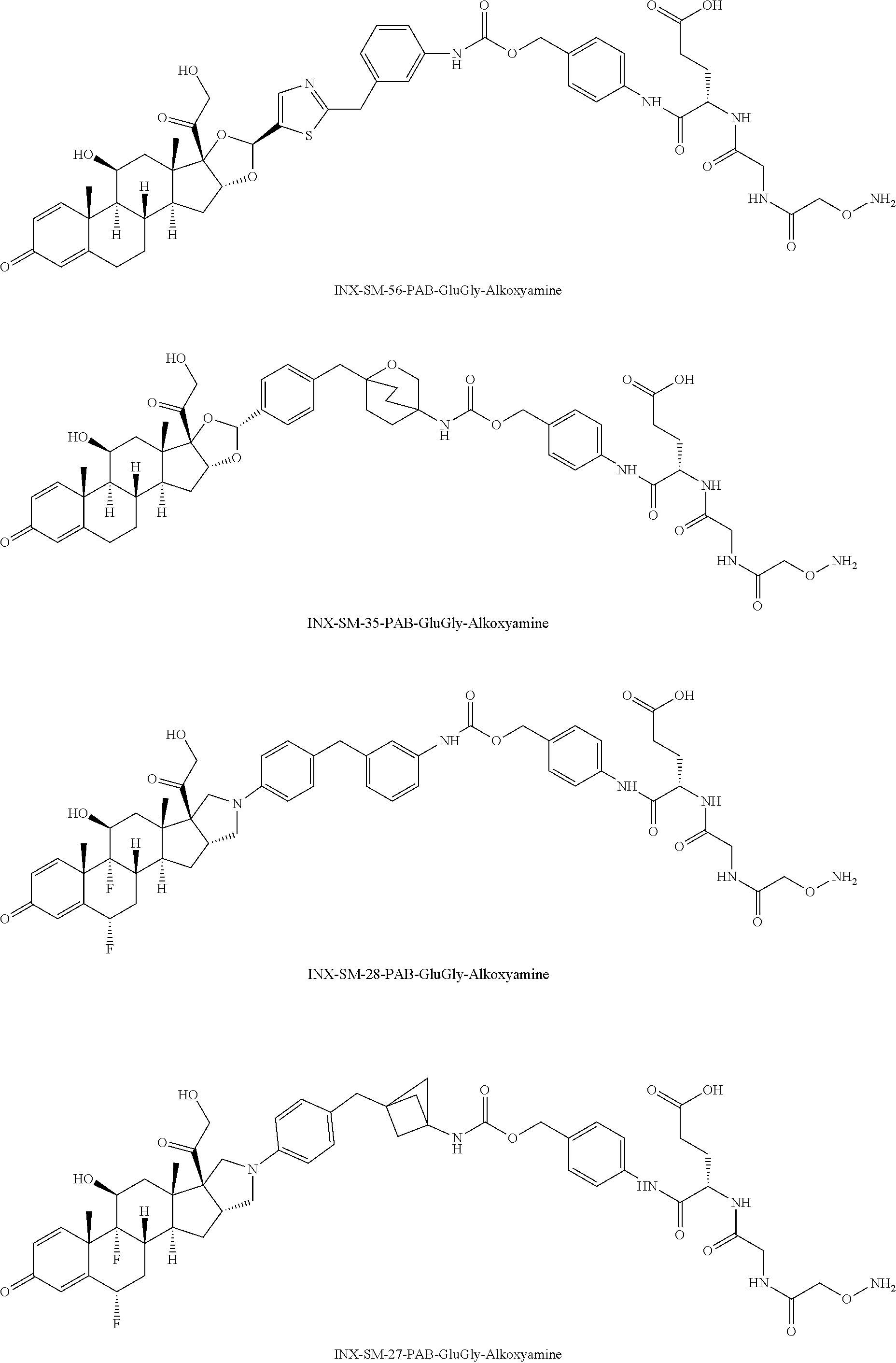

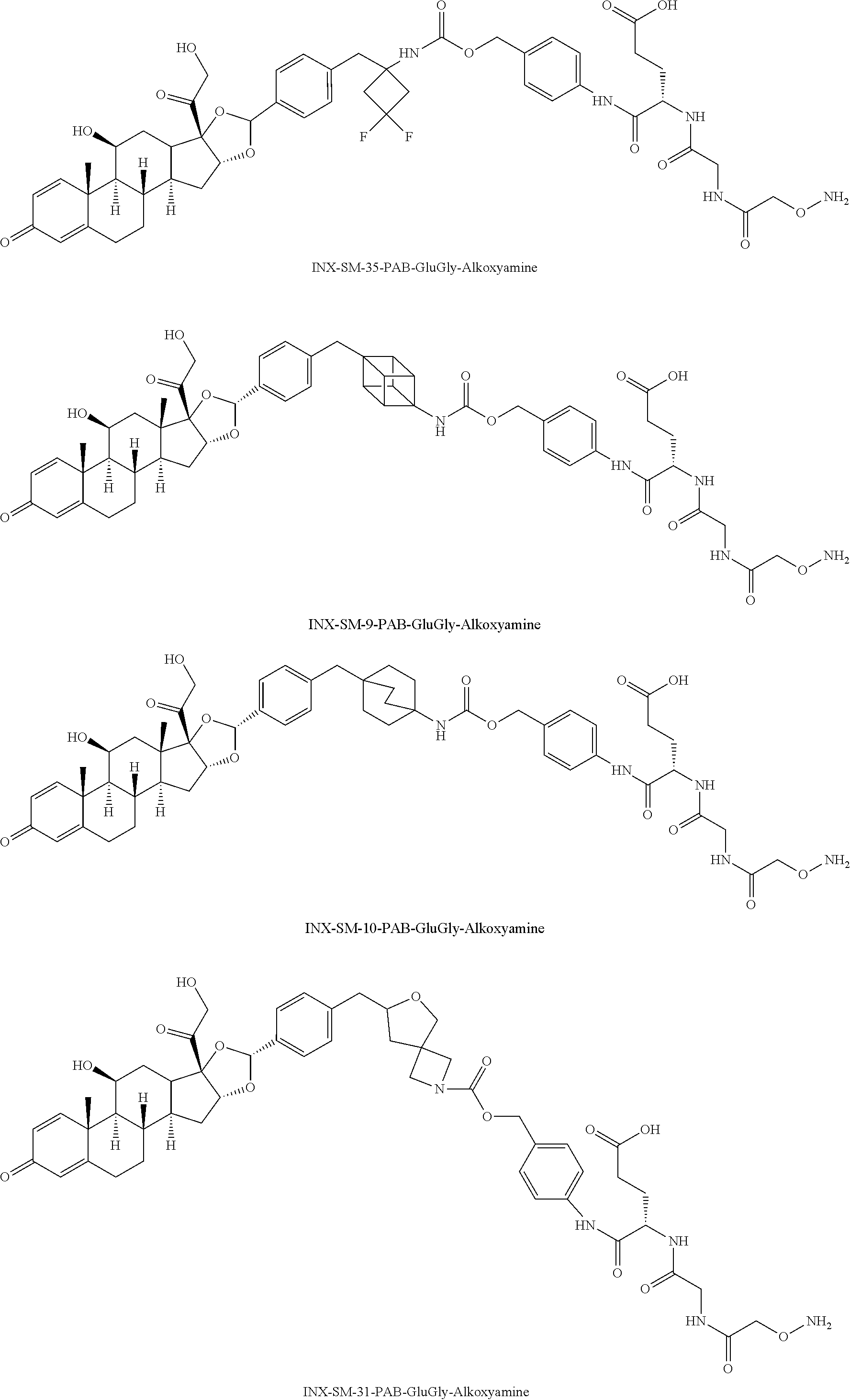

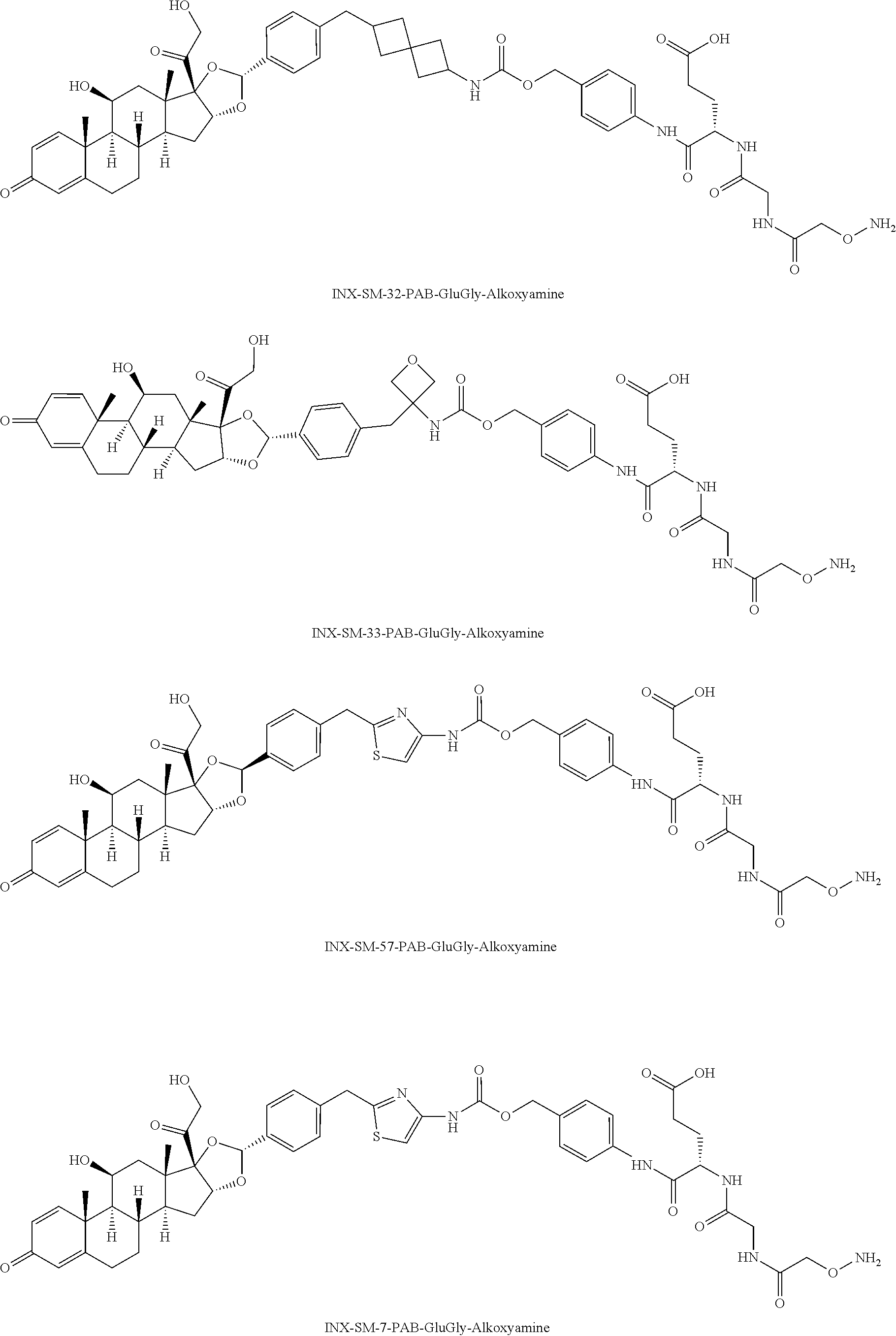

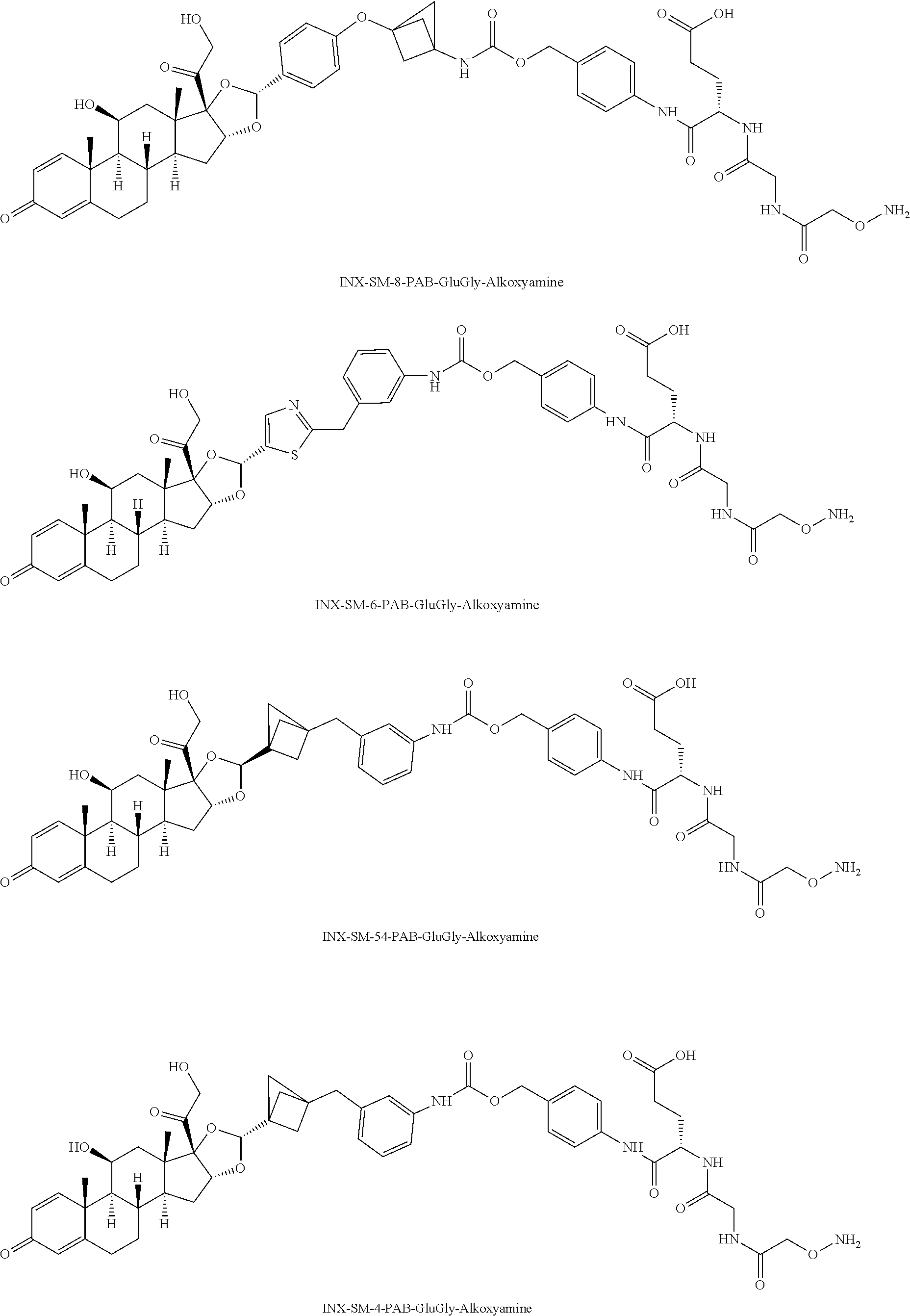

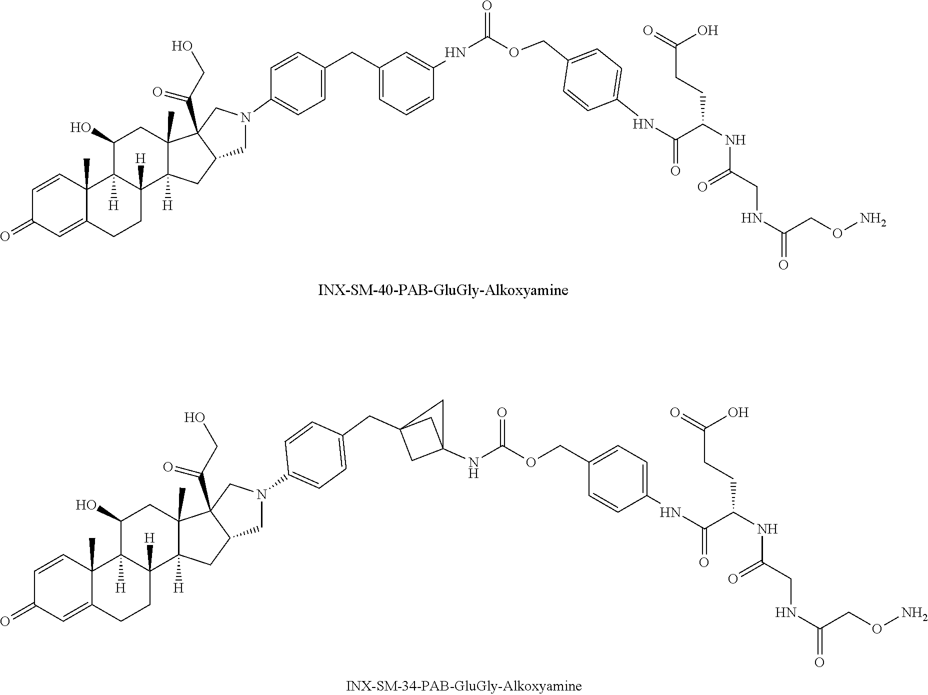

Where,
Linker=Protease cleavable sequence (AA)+Immolative linker Para Amino Benzyl (PAB)
J=Bromoacetyl

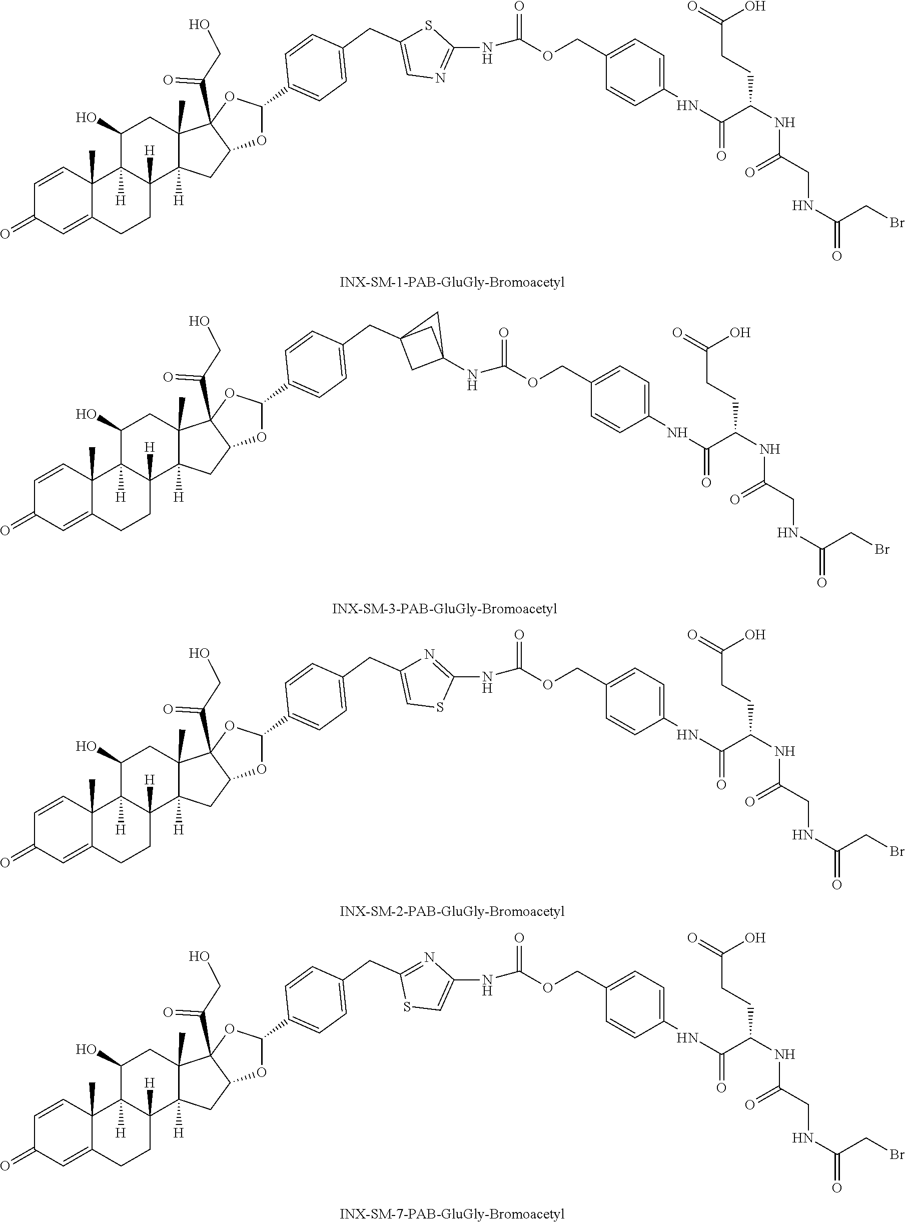

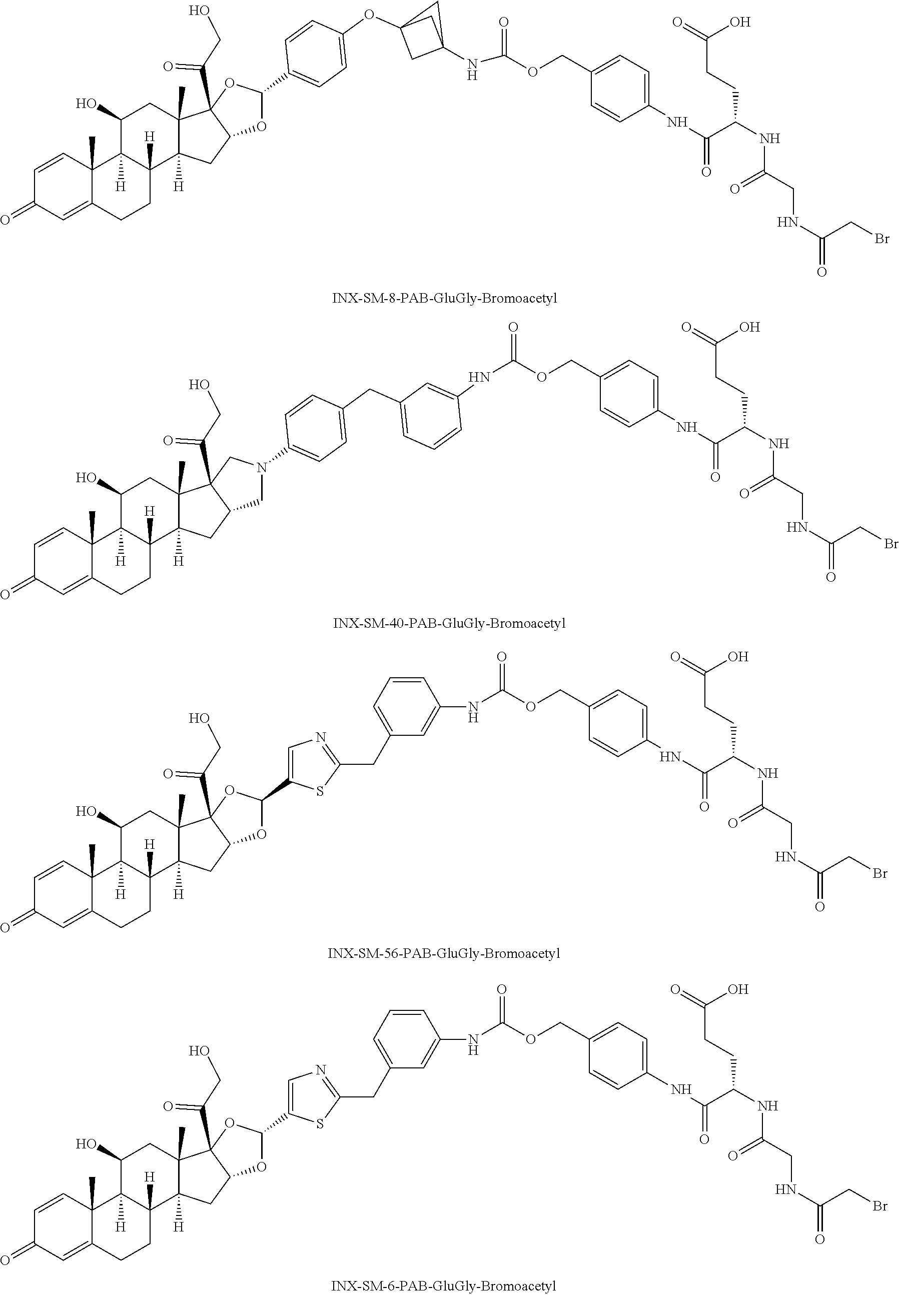

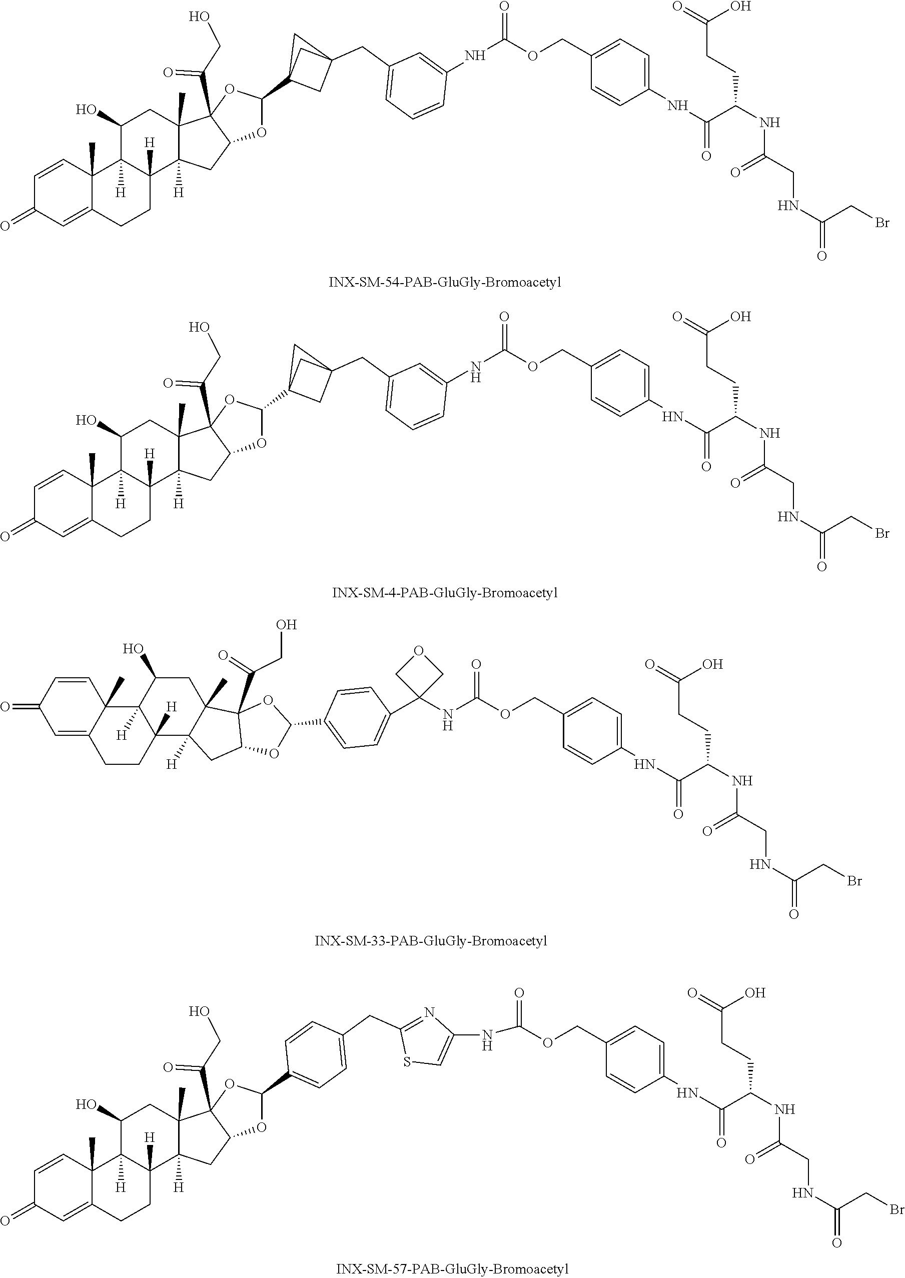

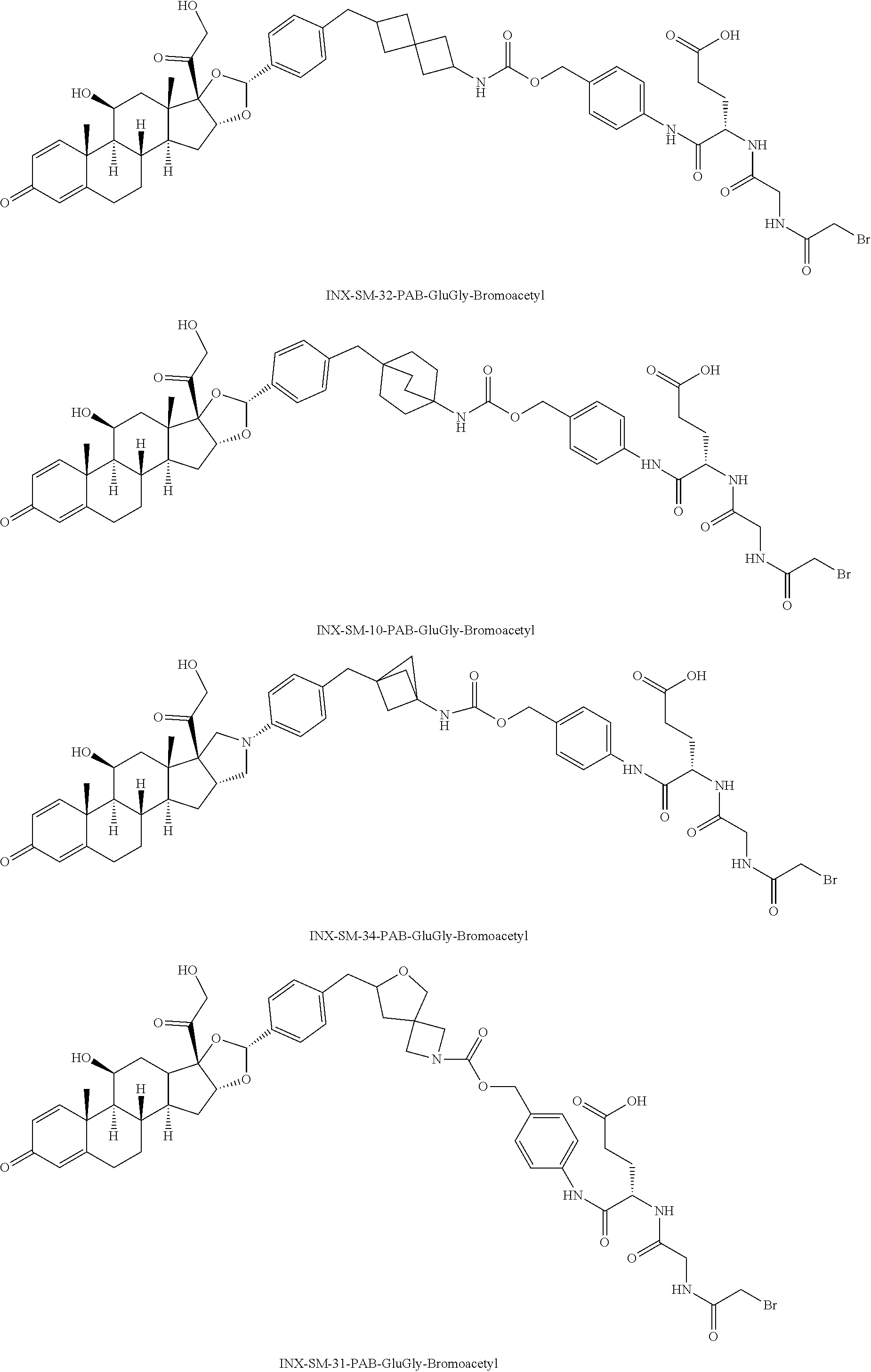

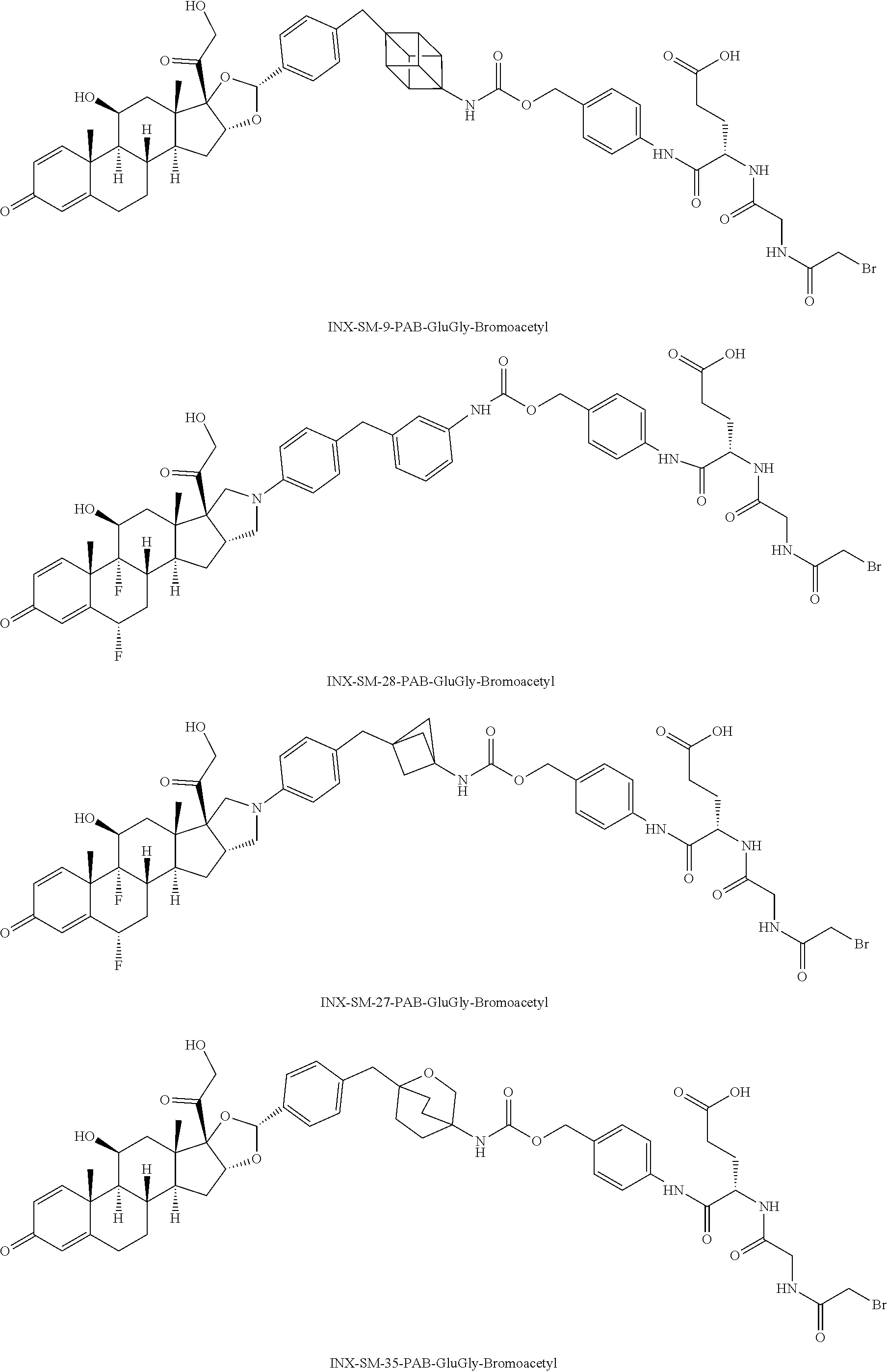

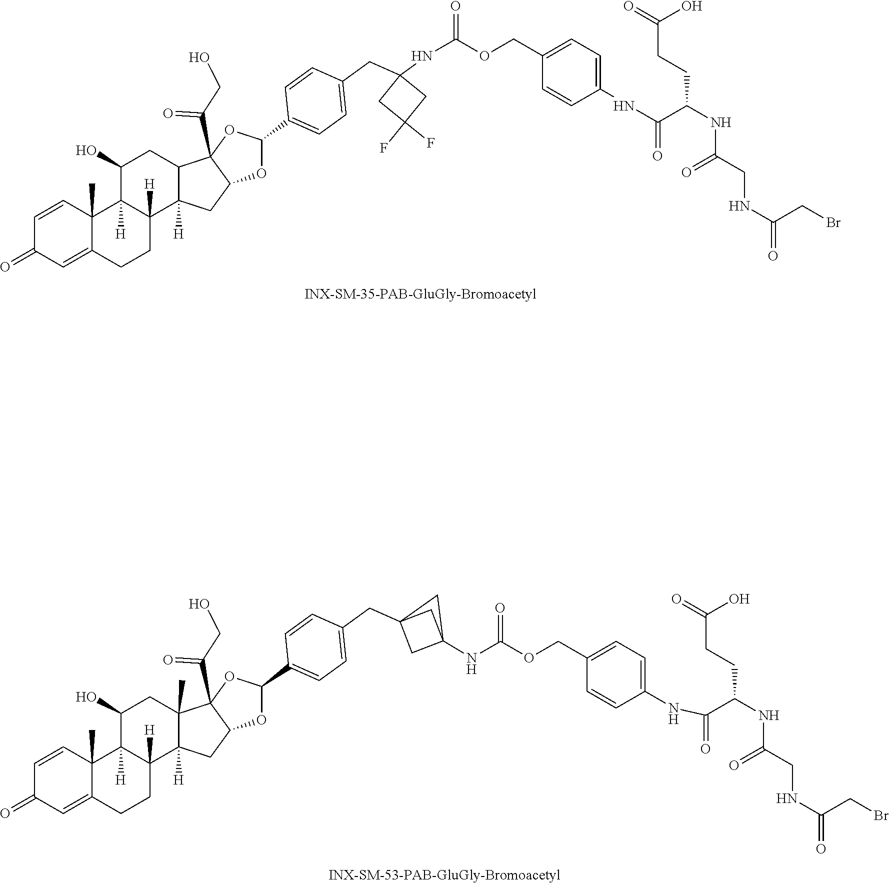

Where,
Linker=Protease cleavable sequence (AA)+Immolative linker Para Amino Benzyl (PAB)
J=Dibenzocyclooctyne

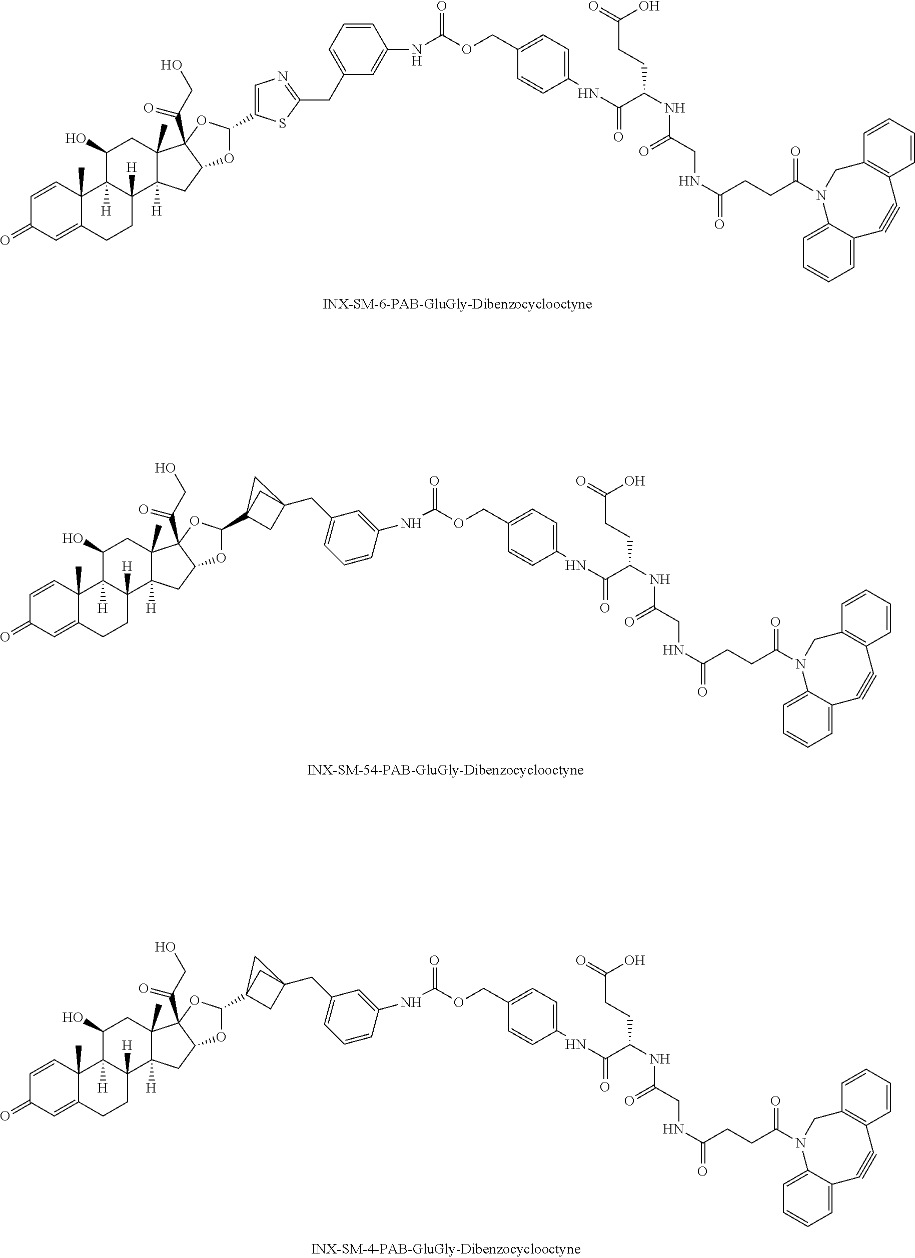

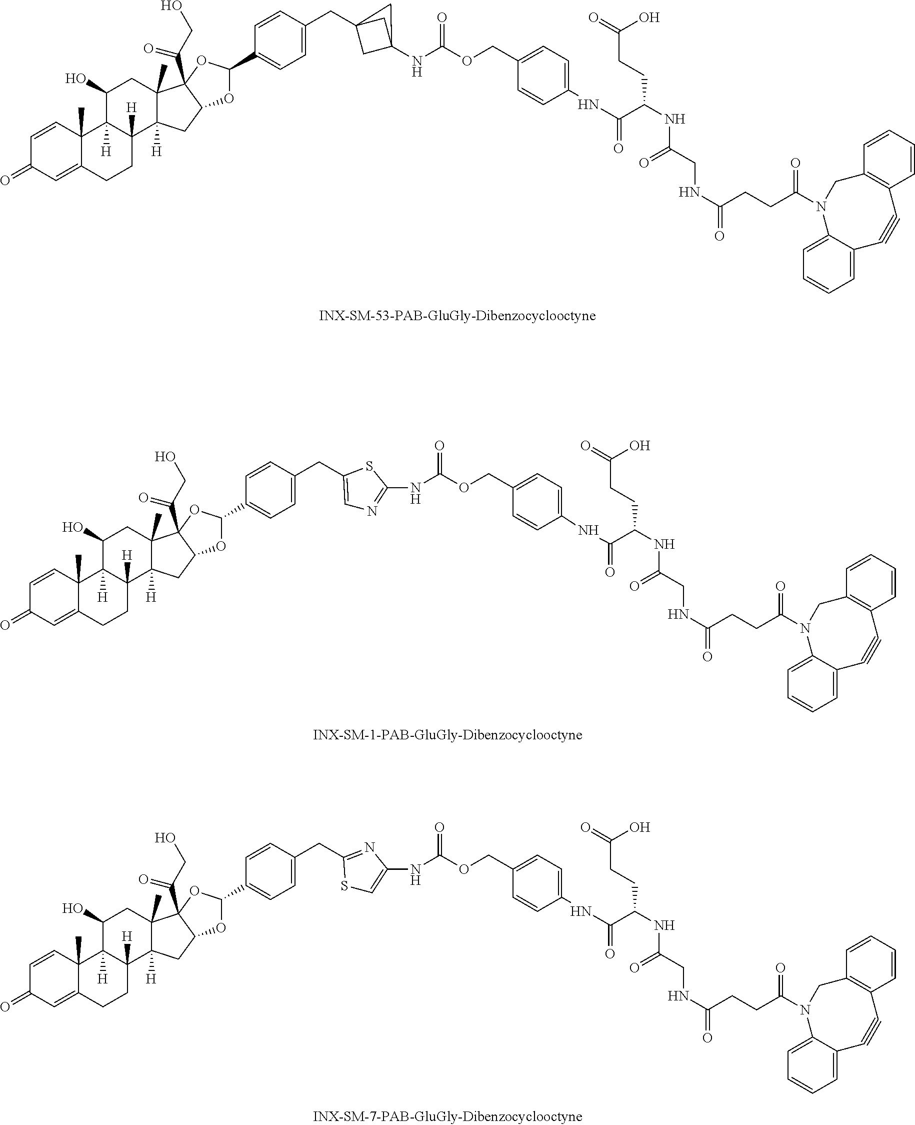

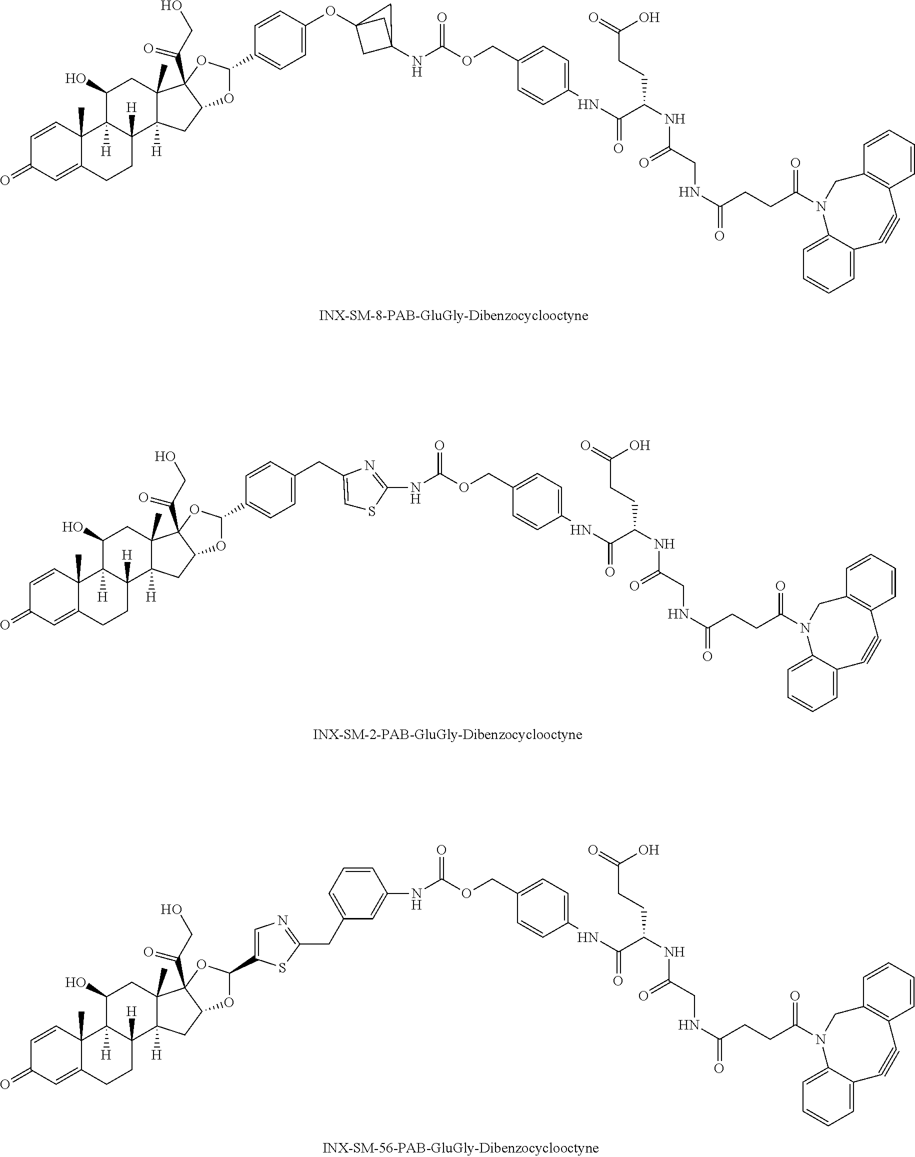

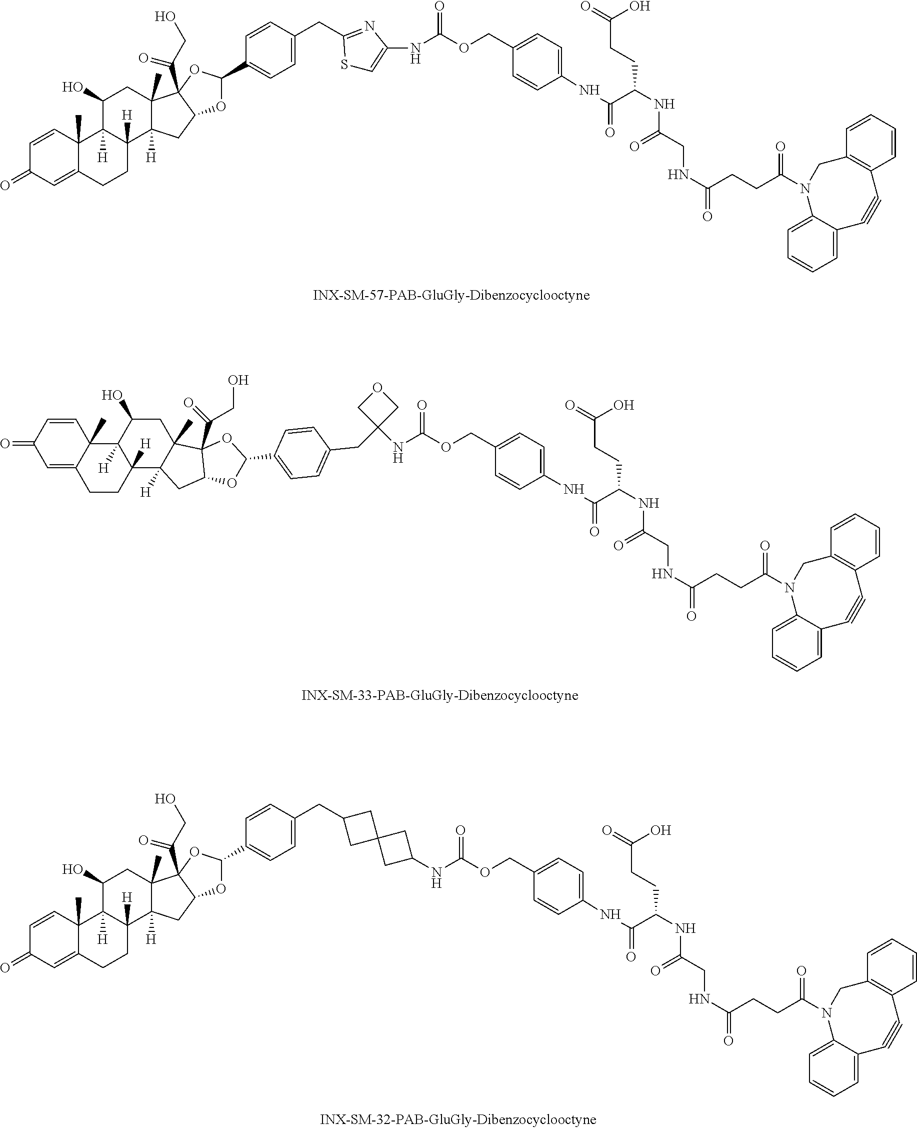

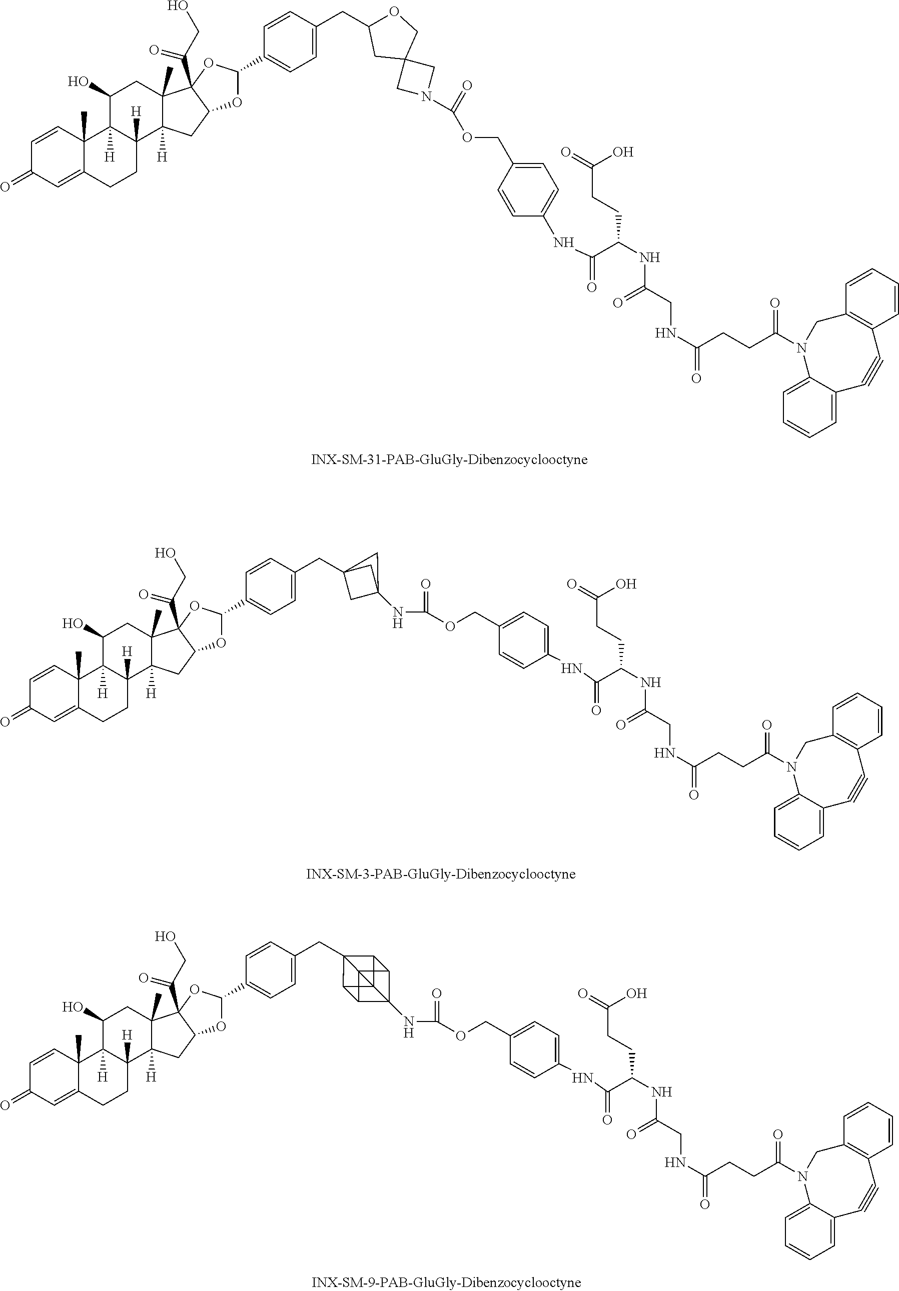

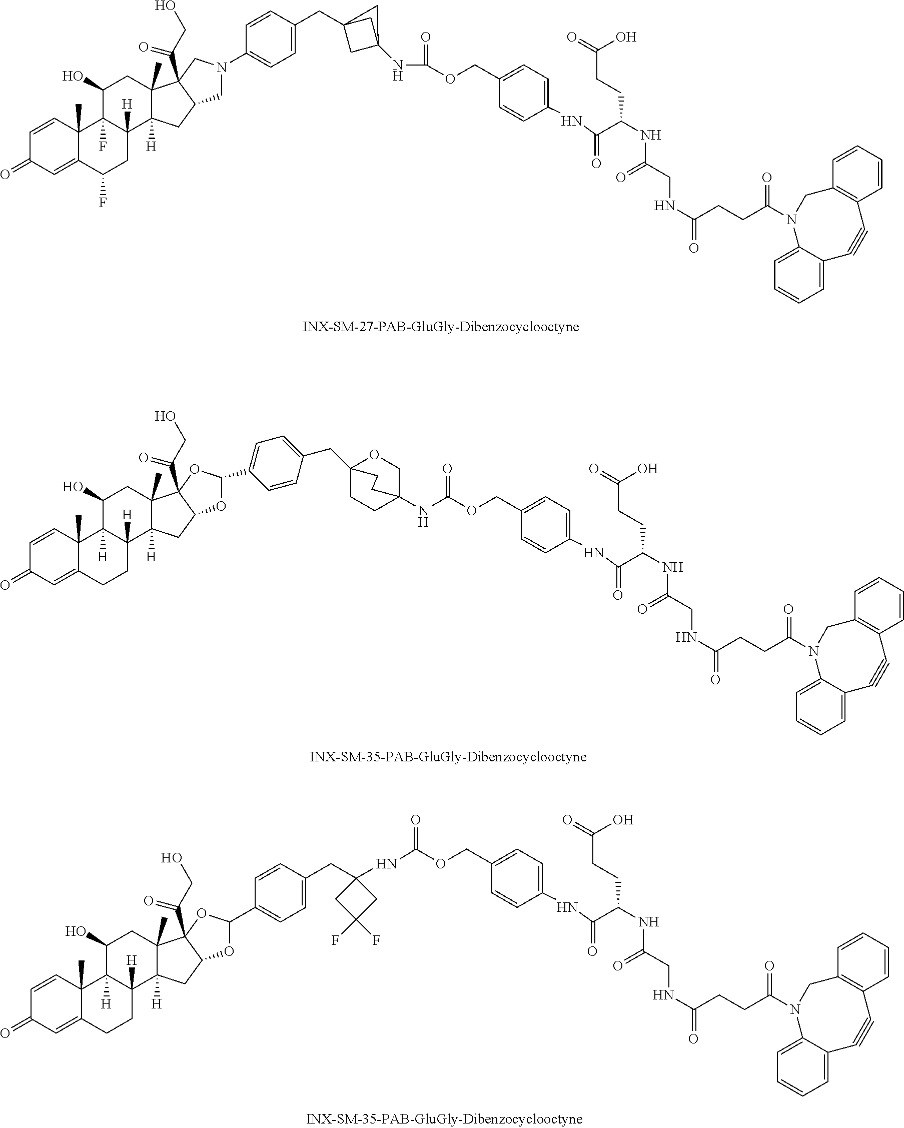

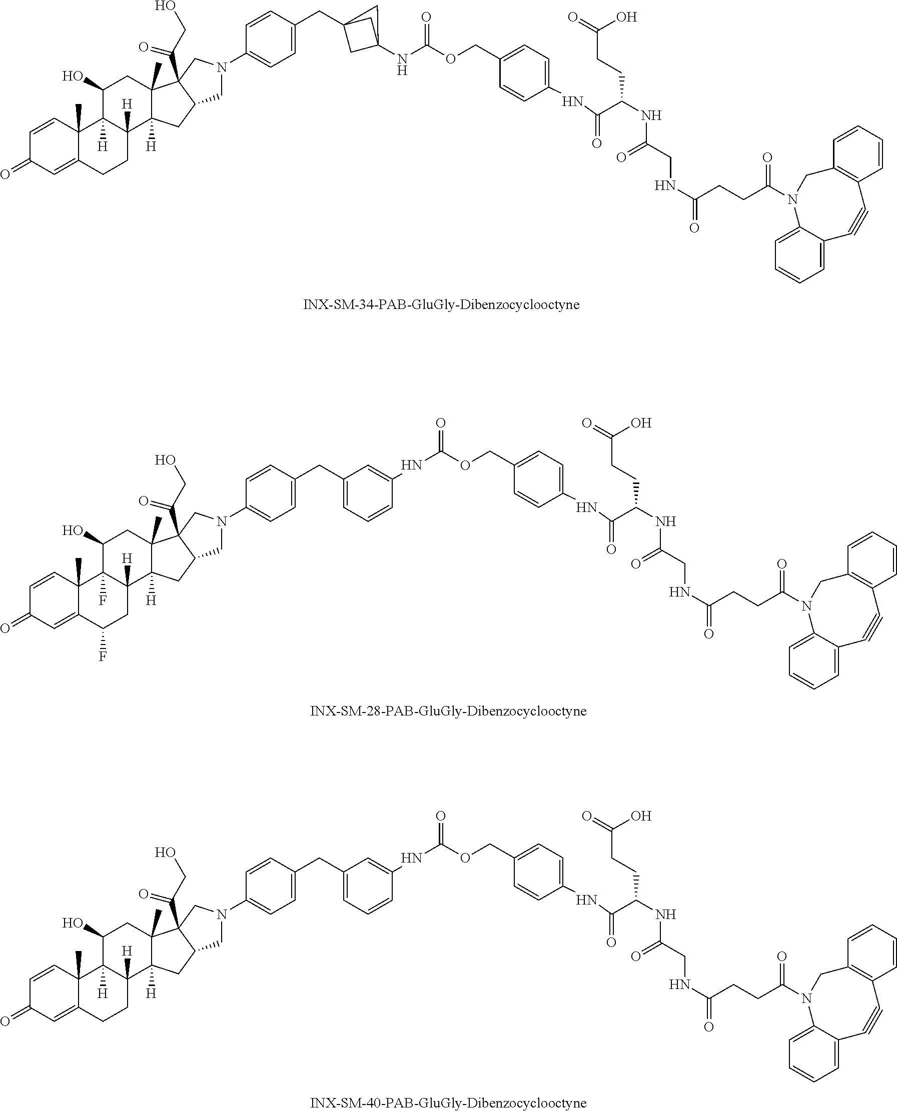

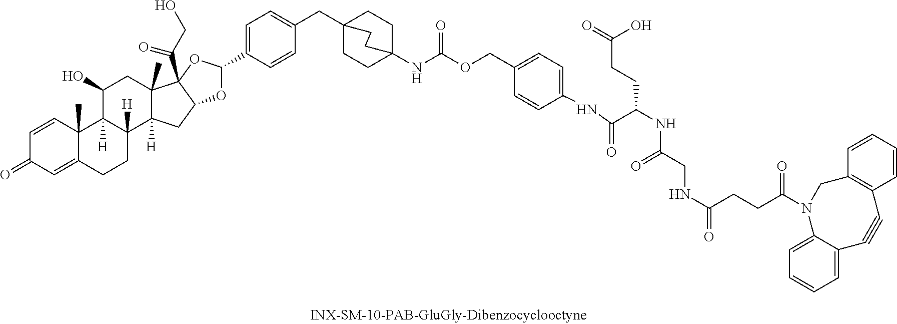

Where,
Linker=Protease cleavable sequence (AA)+Immolative linker Para Amino Benzyl (PAB)
J=Hydroxysuccinimide

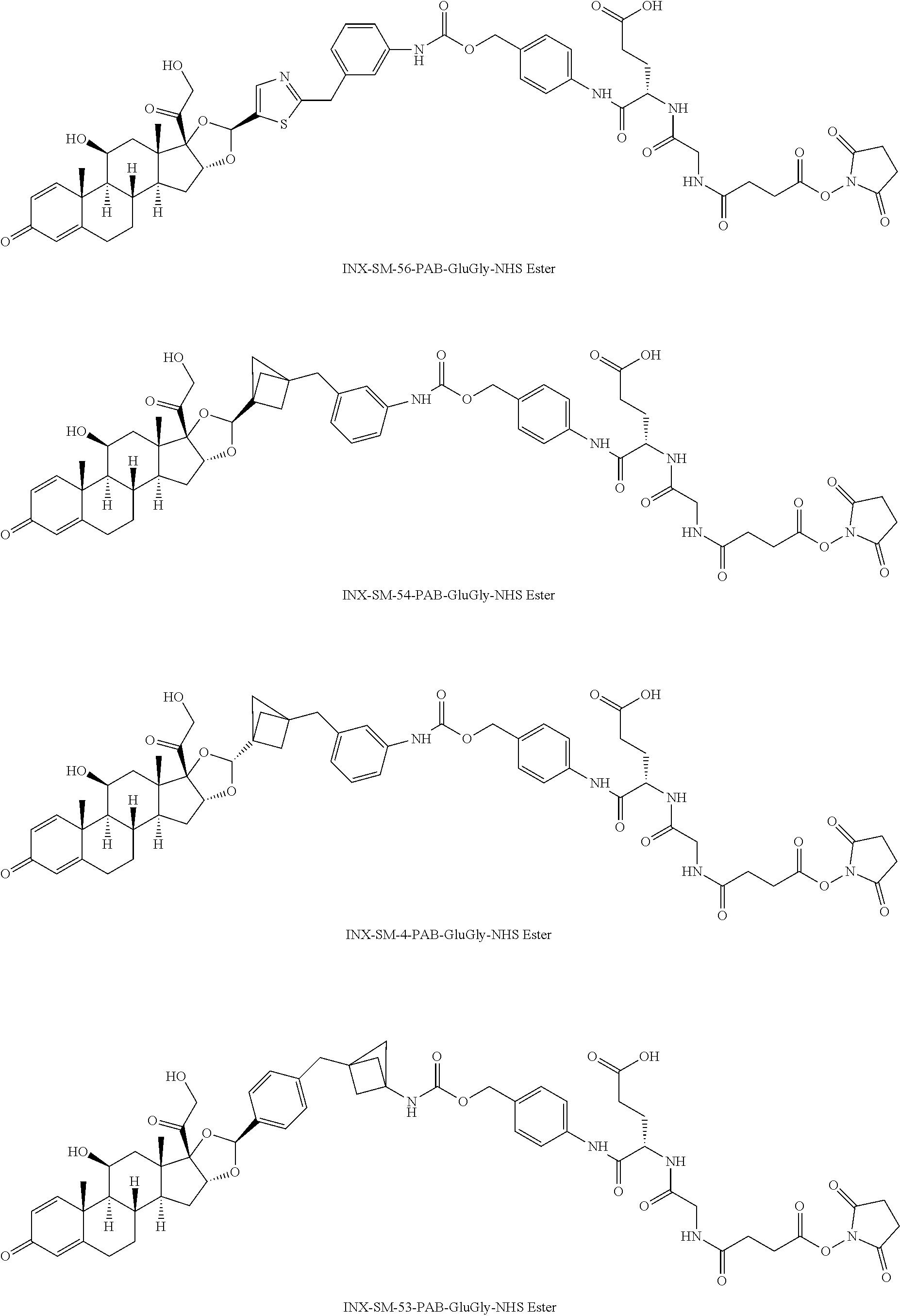

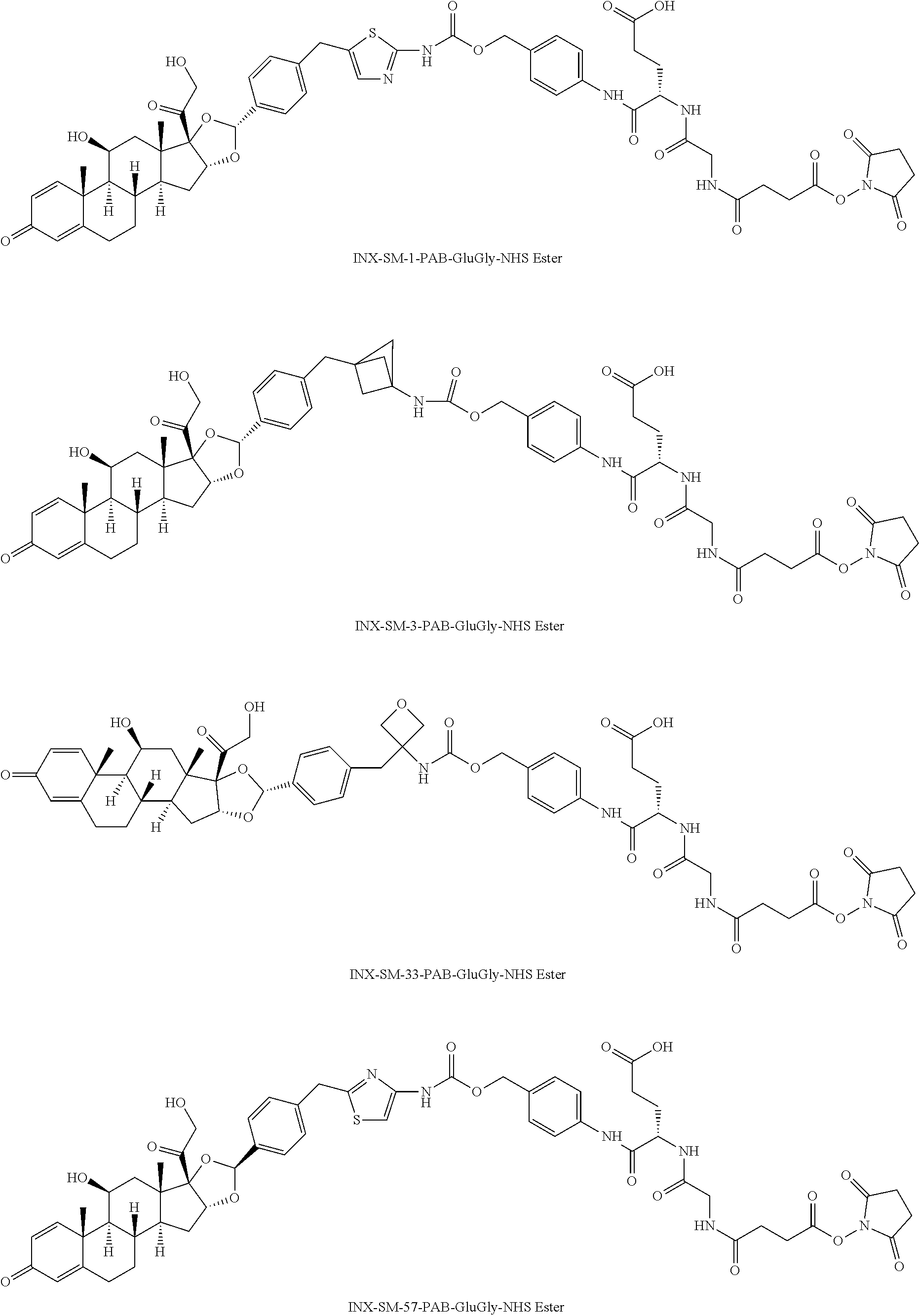

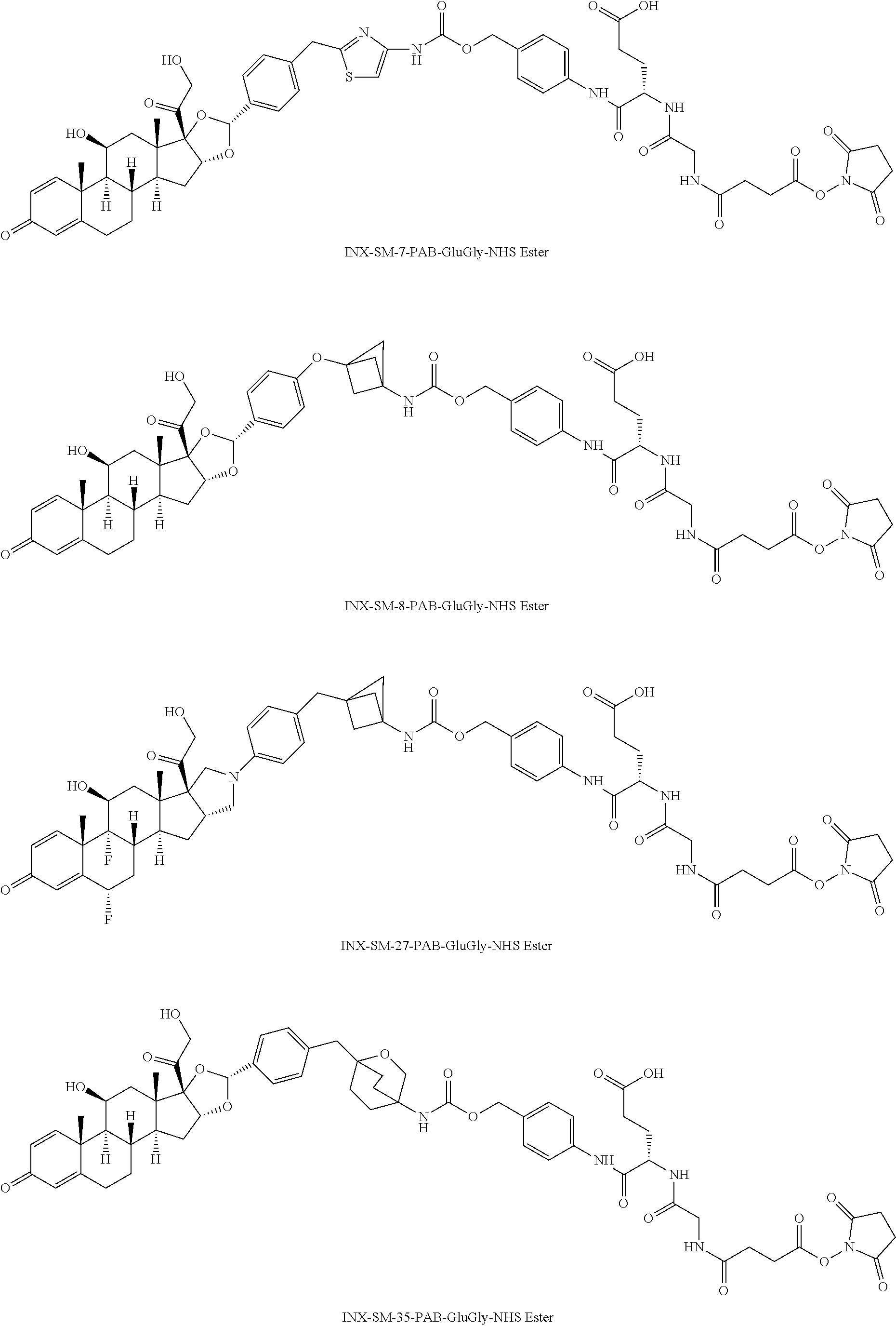

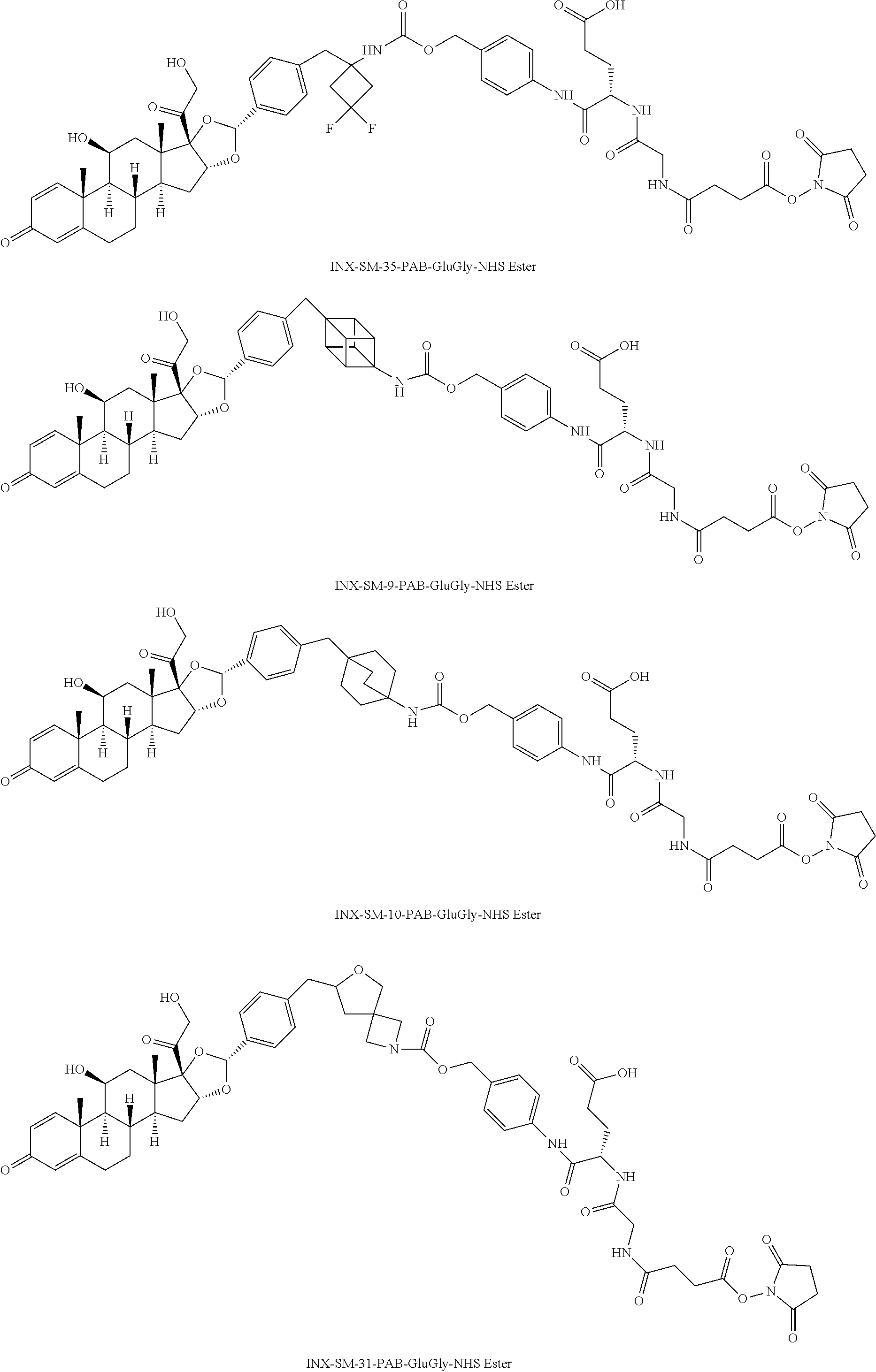

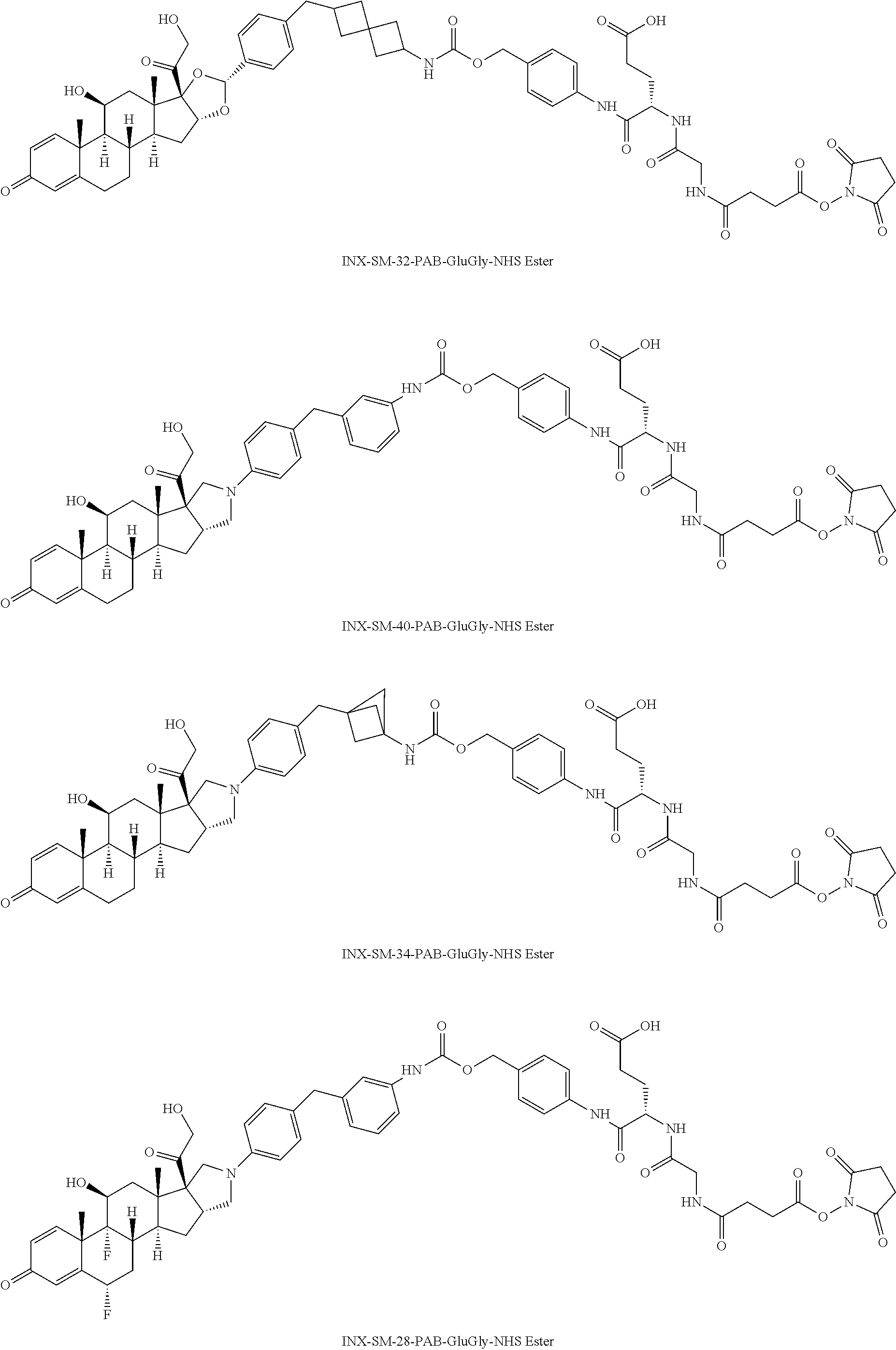

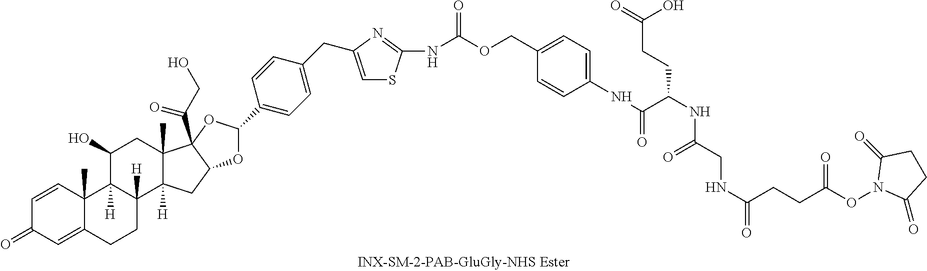

Where,
Linker=Protease cleavable sequence (AA)+Immolative linker Para Amino Benzyl (PAB)
J=Maleimide

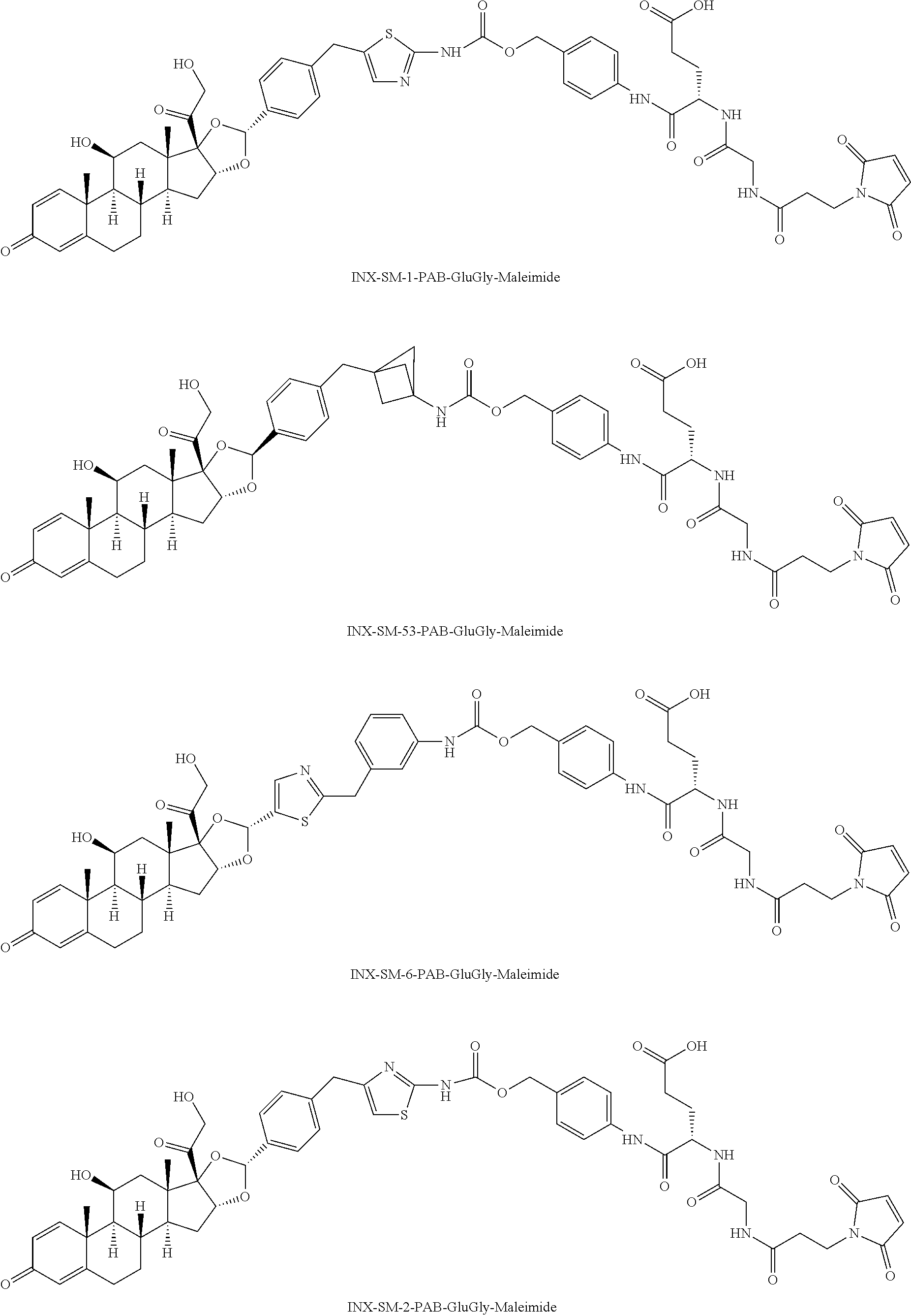

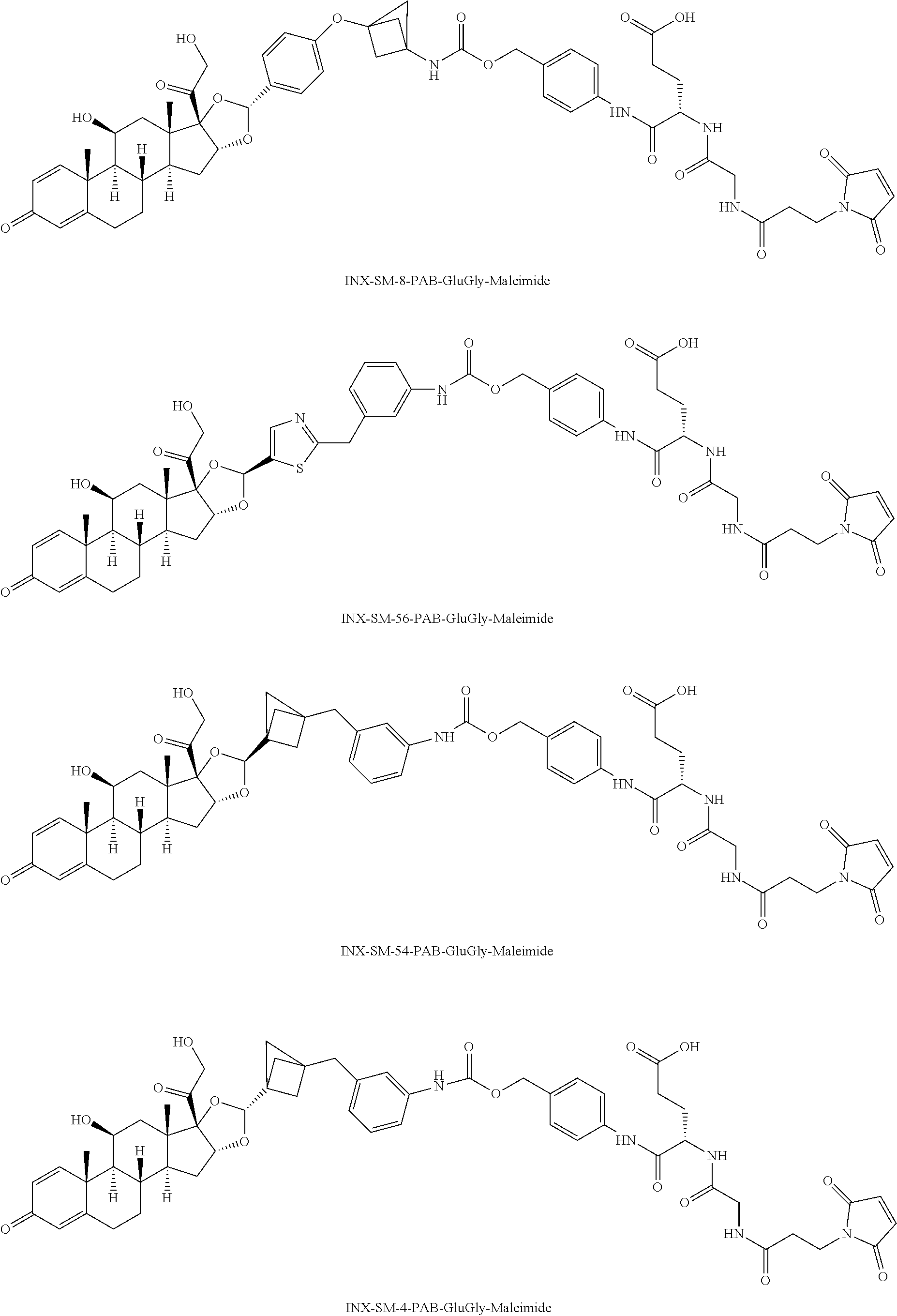

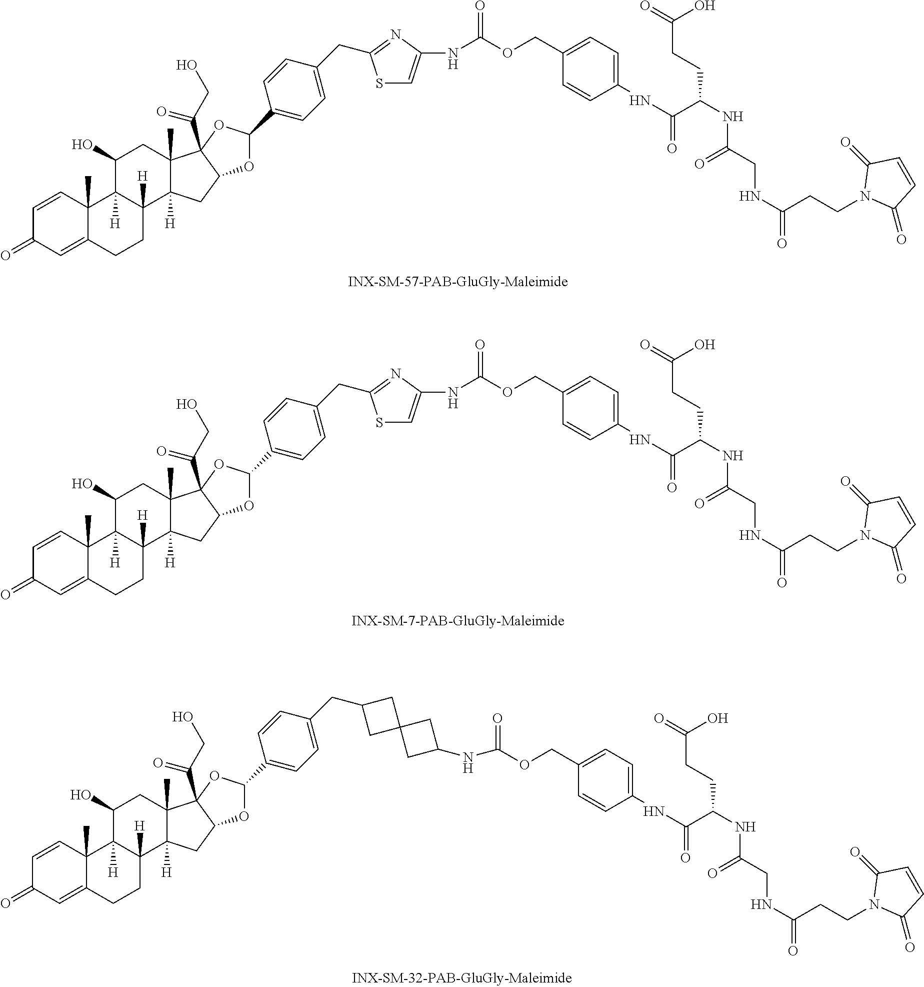

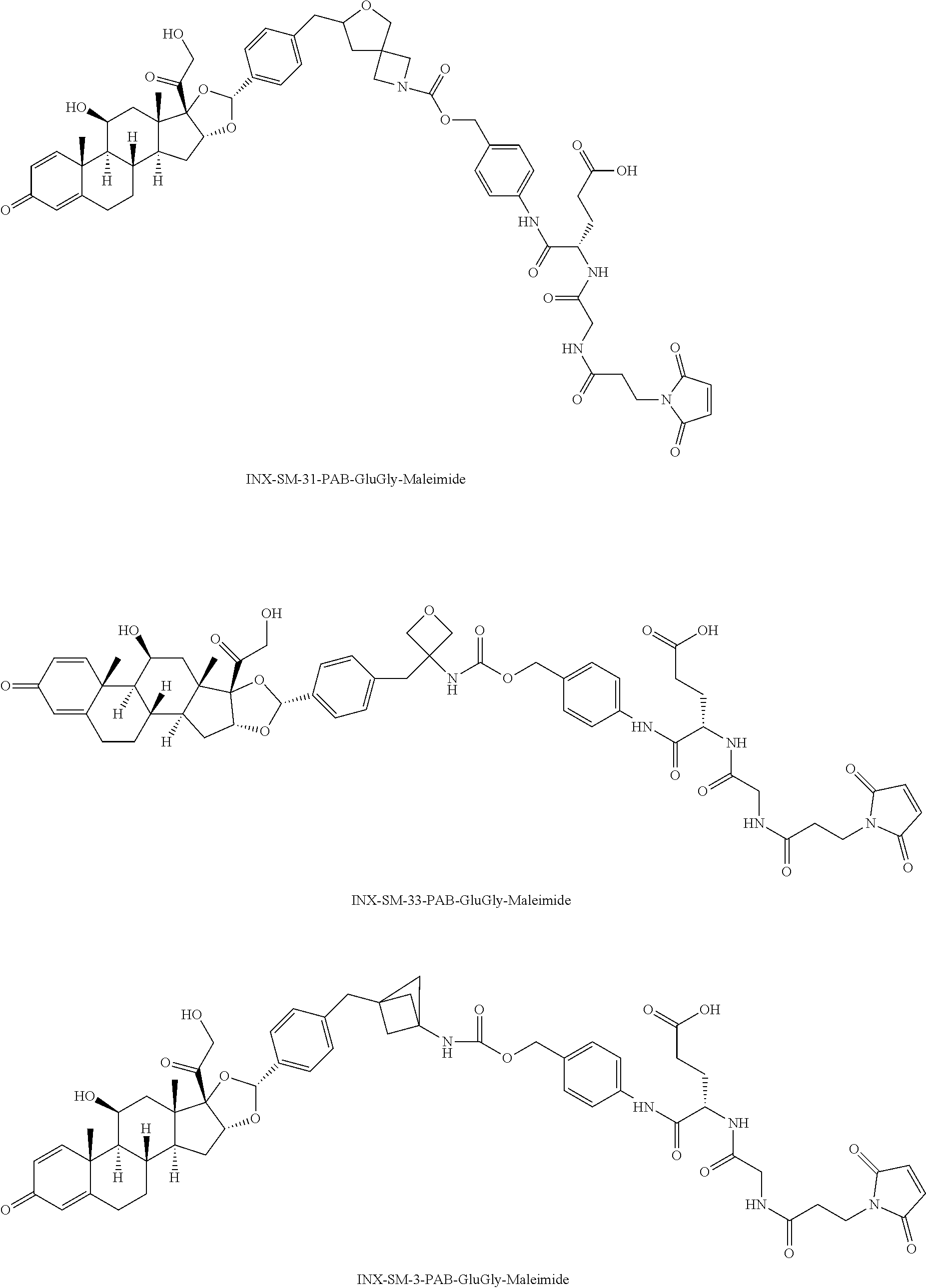

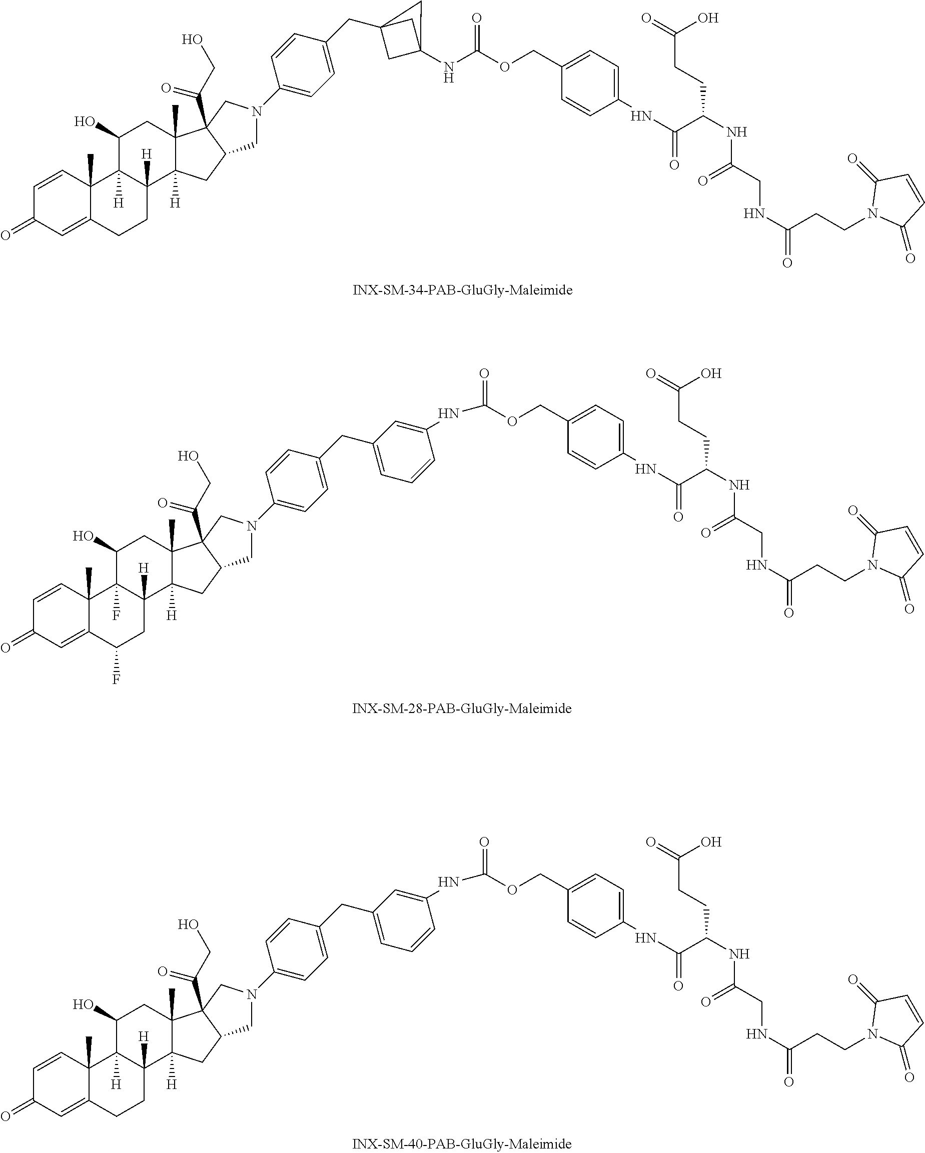

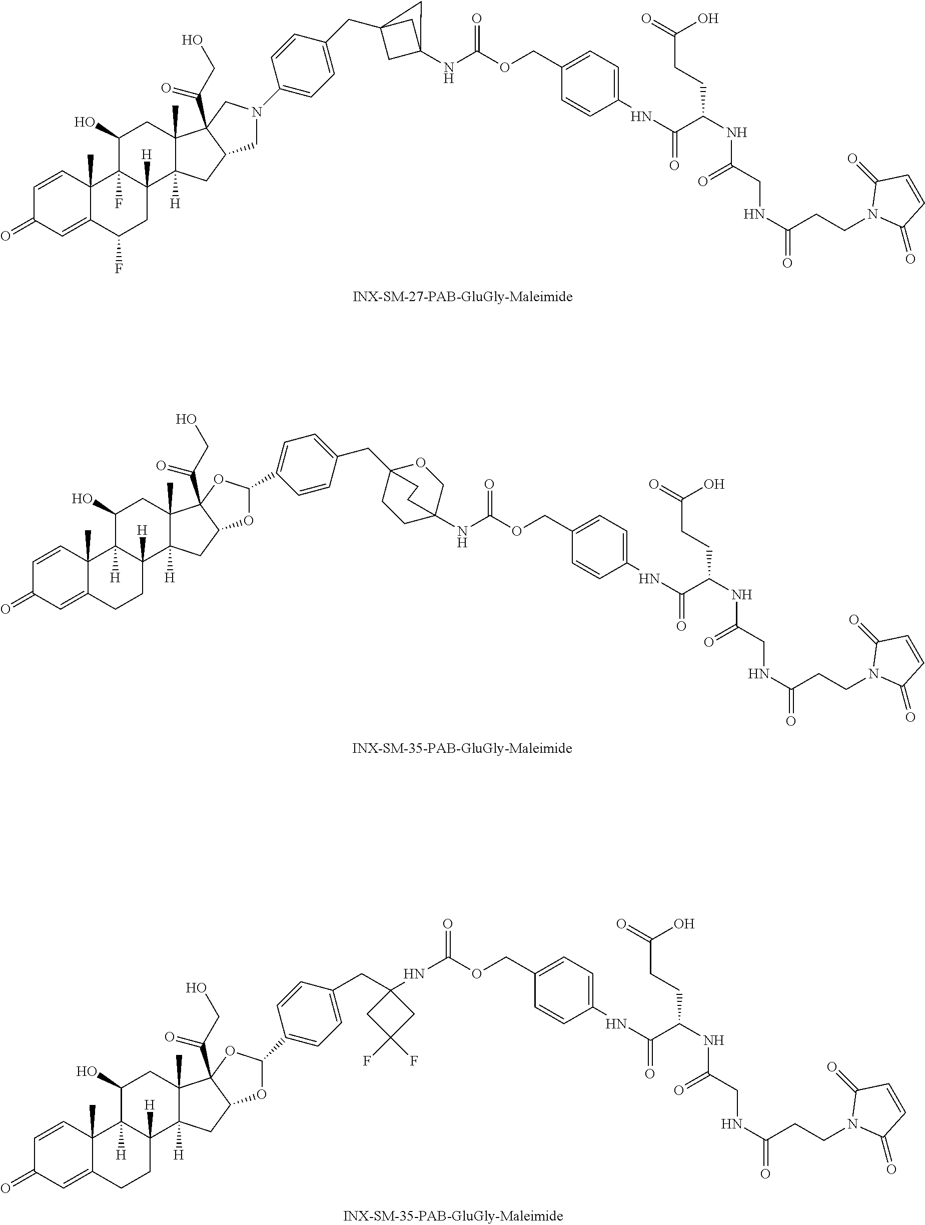

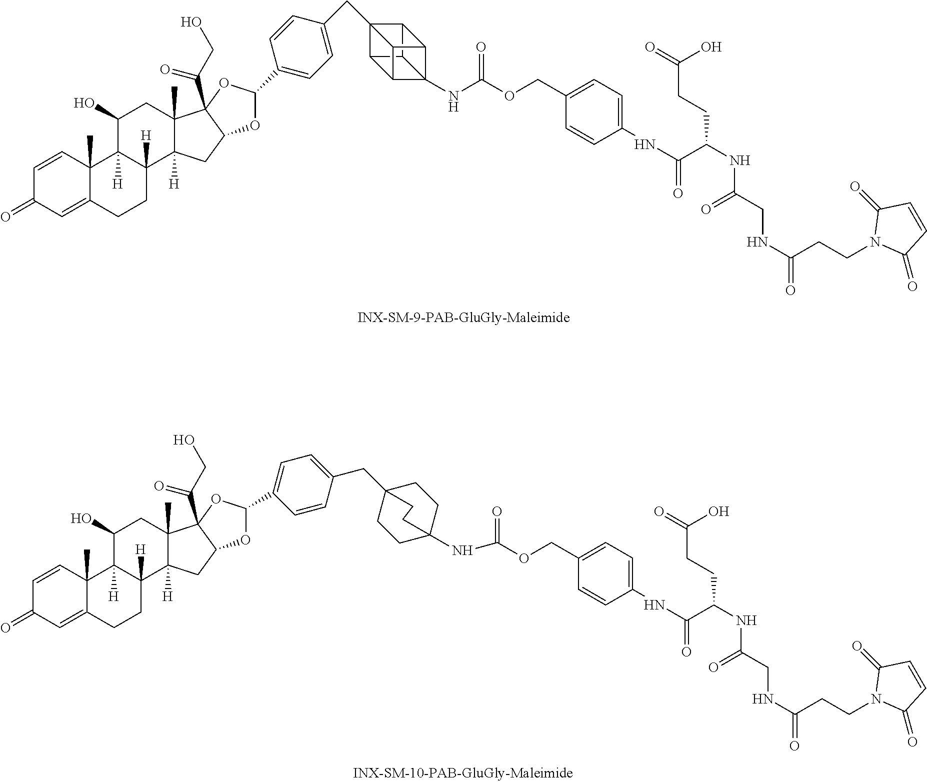

Where,
Linker=Protease cleavable sequence (AA)+Immolative linker Para Amino Benzyl (PAB)
J=Tetrazine

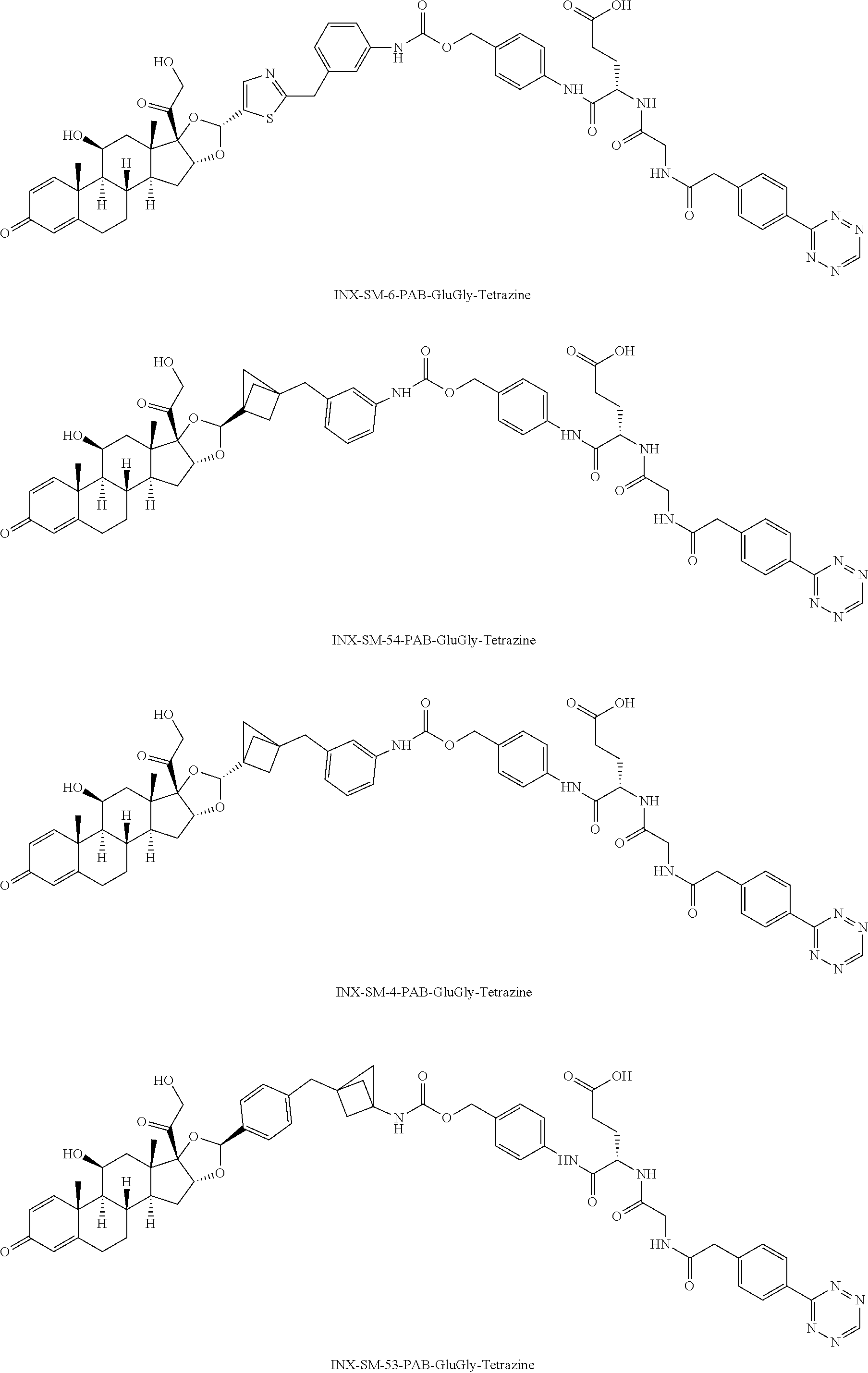

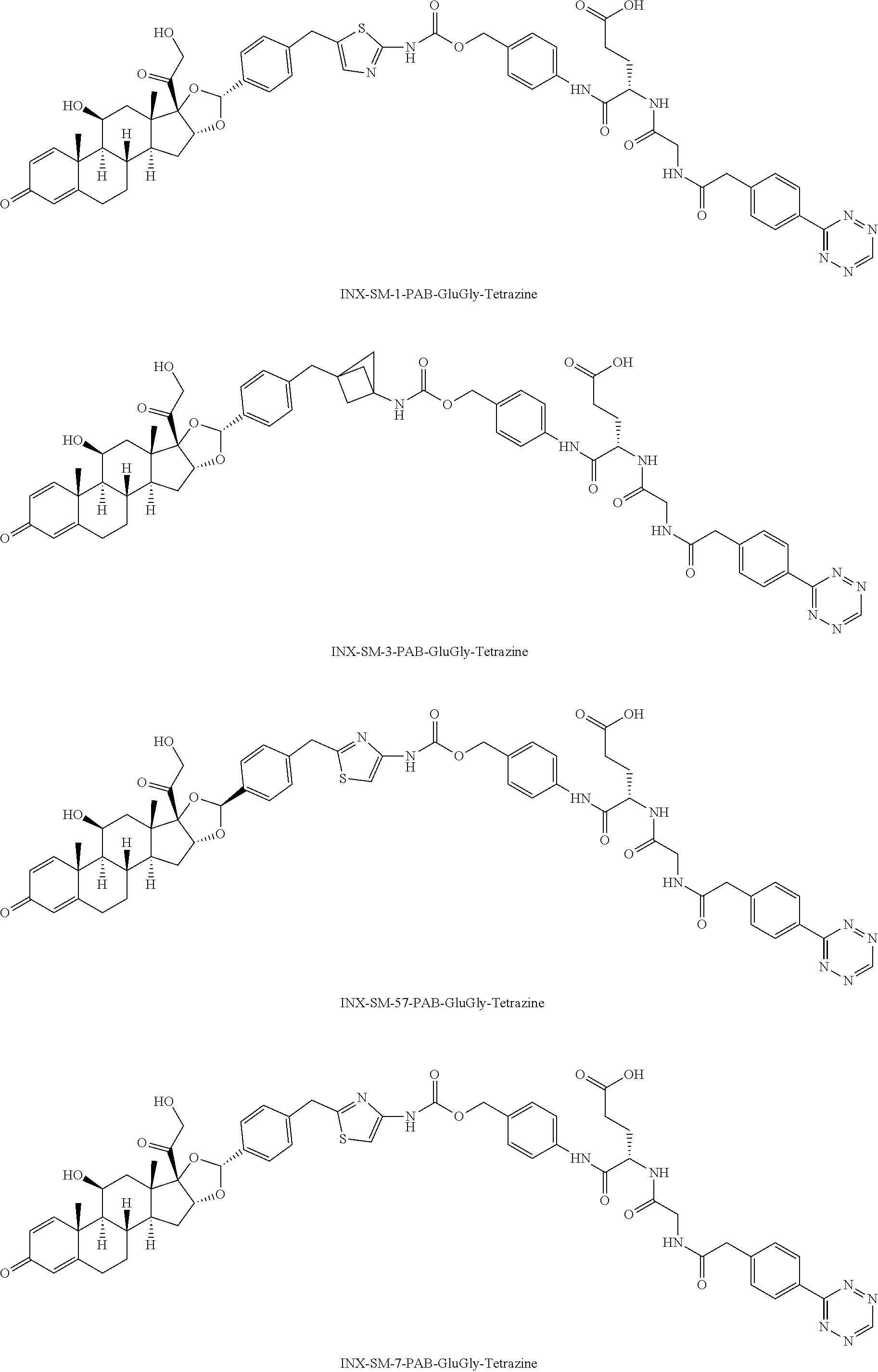

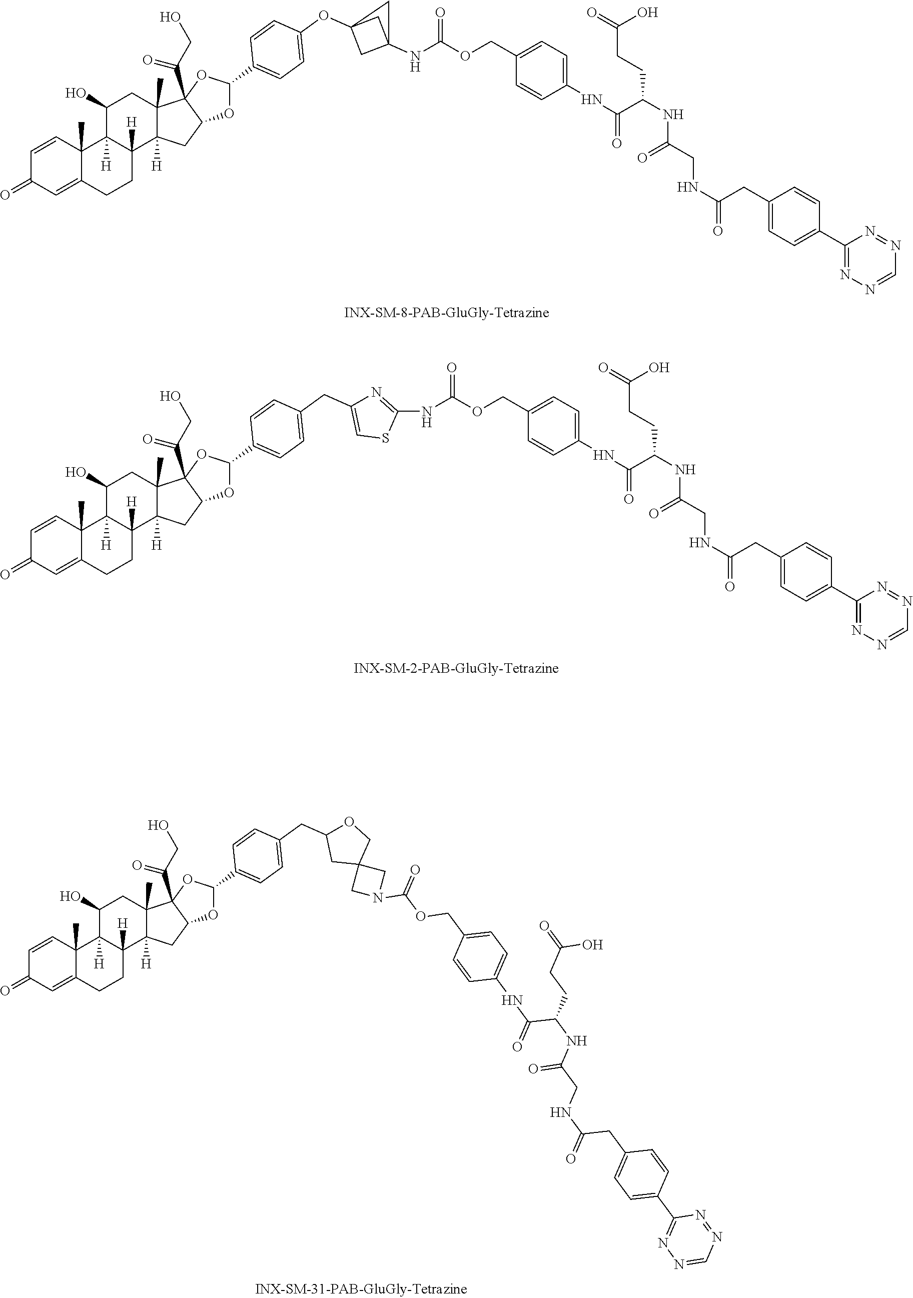

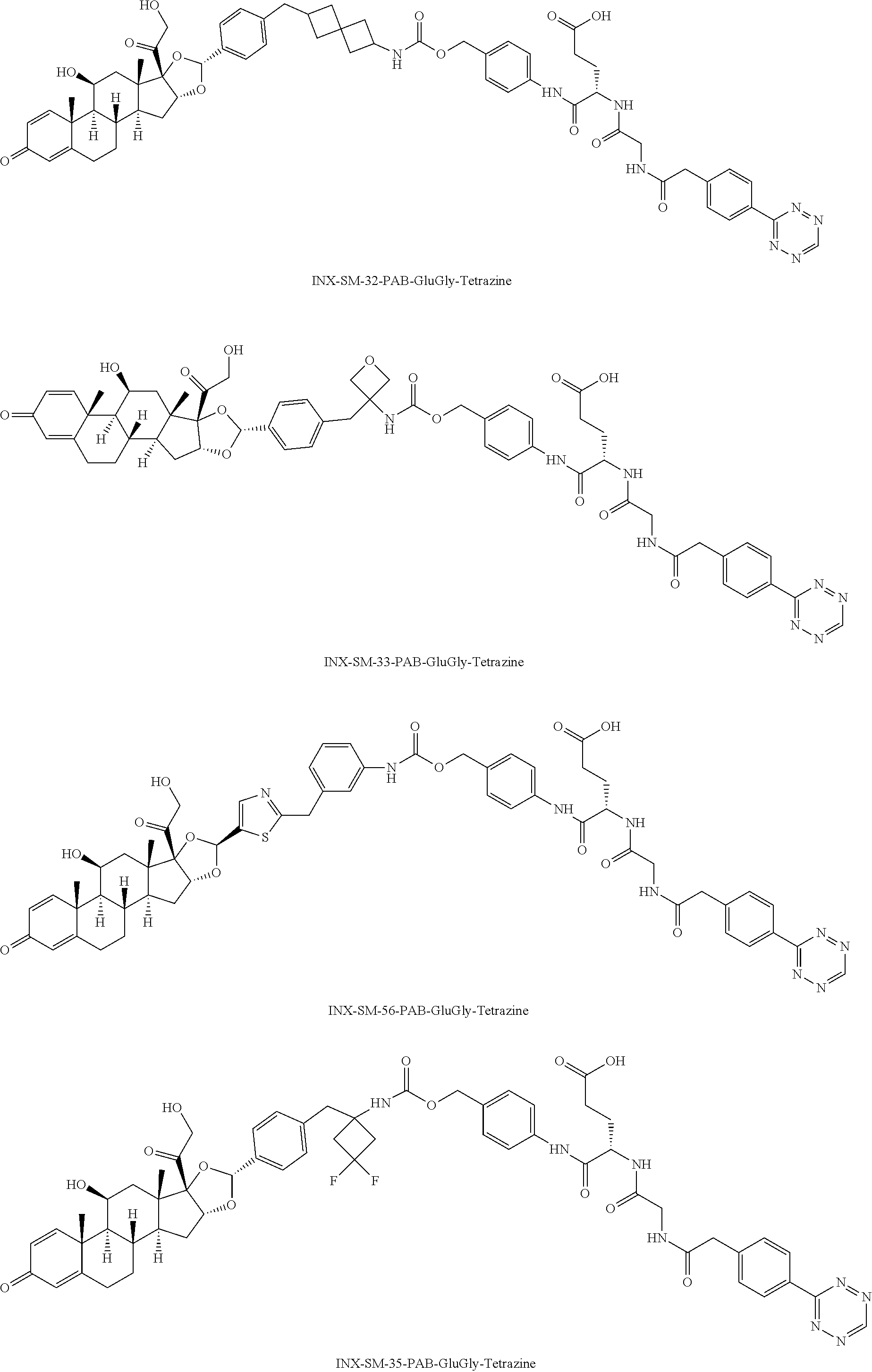

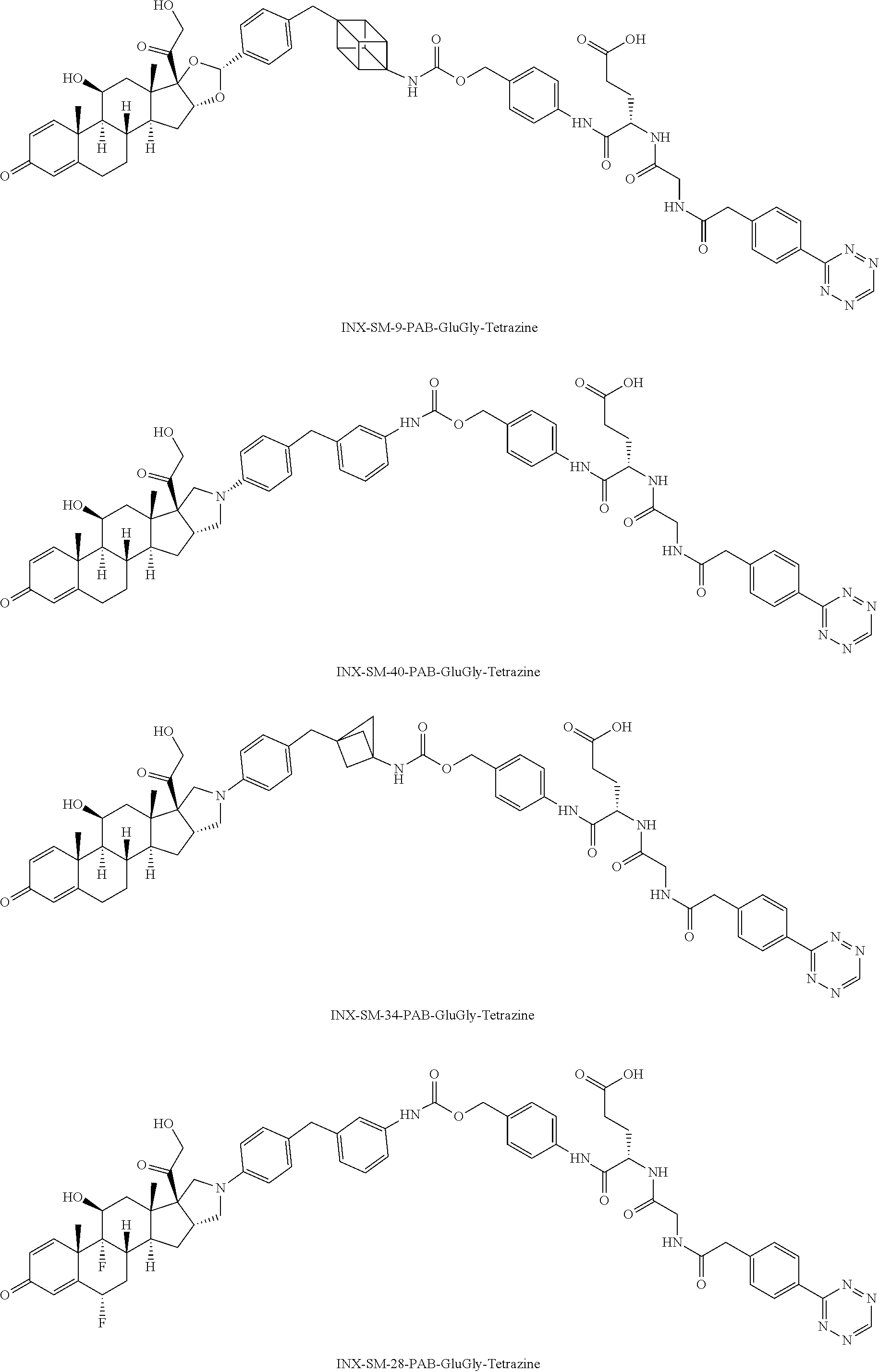

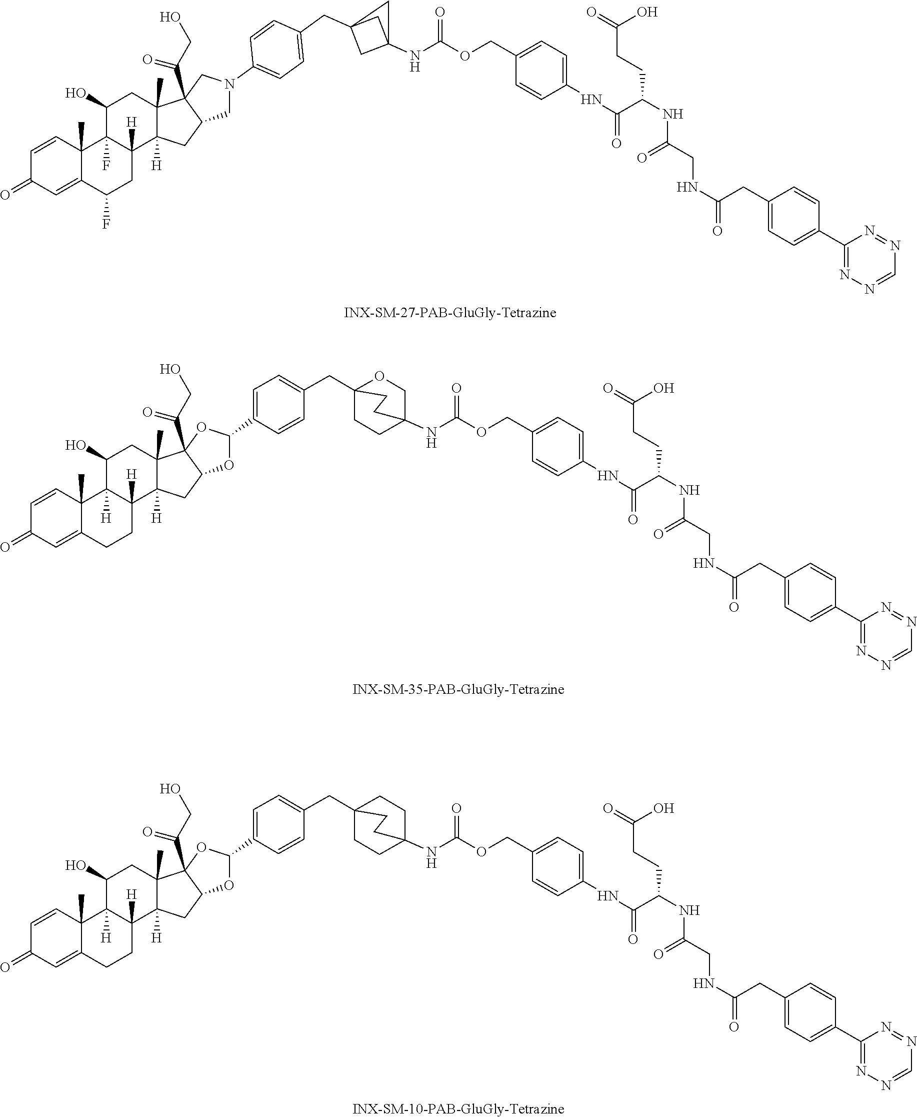

Where,
Linker=Protease cleavable sequence (AA)+Immolative linker Para Amino Benzyl (PAB)
J=Amine

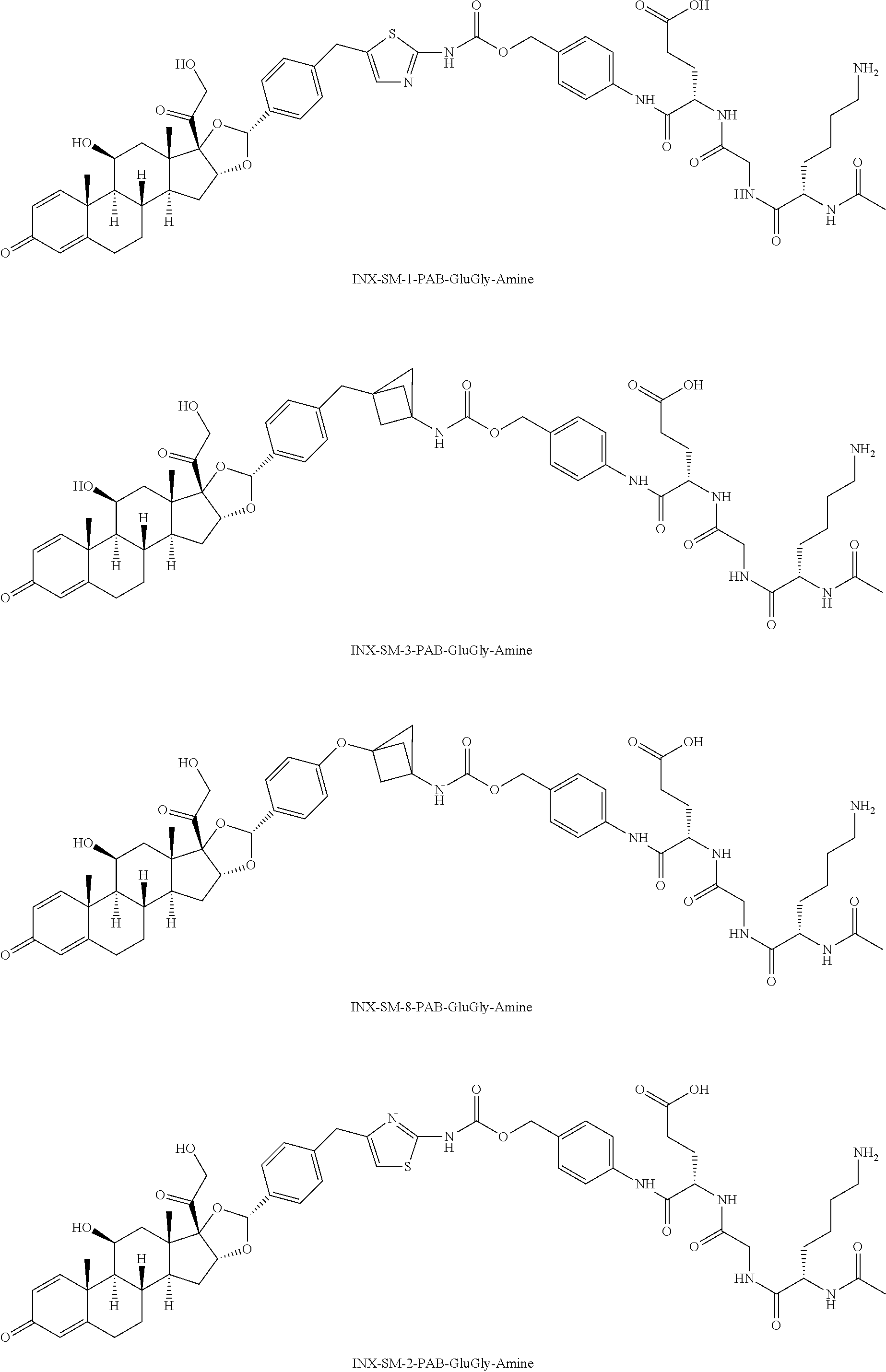

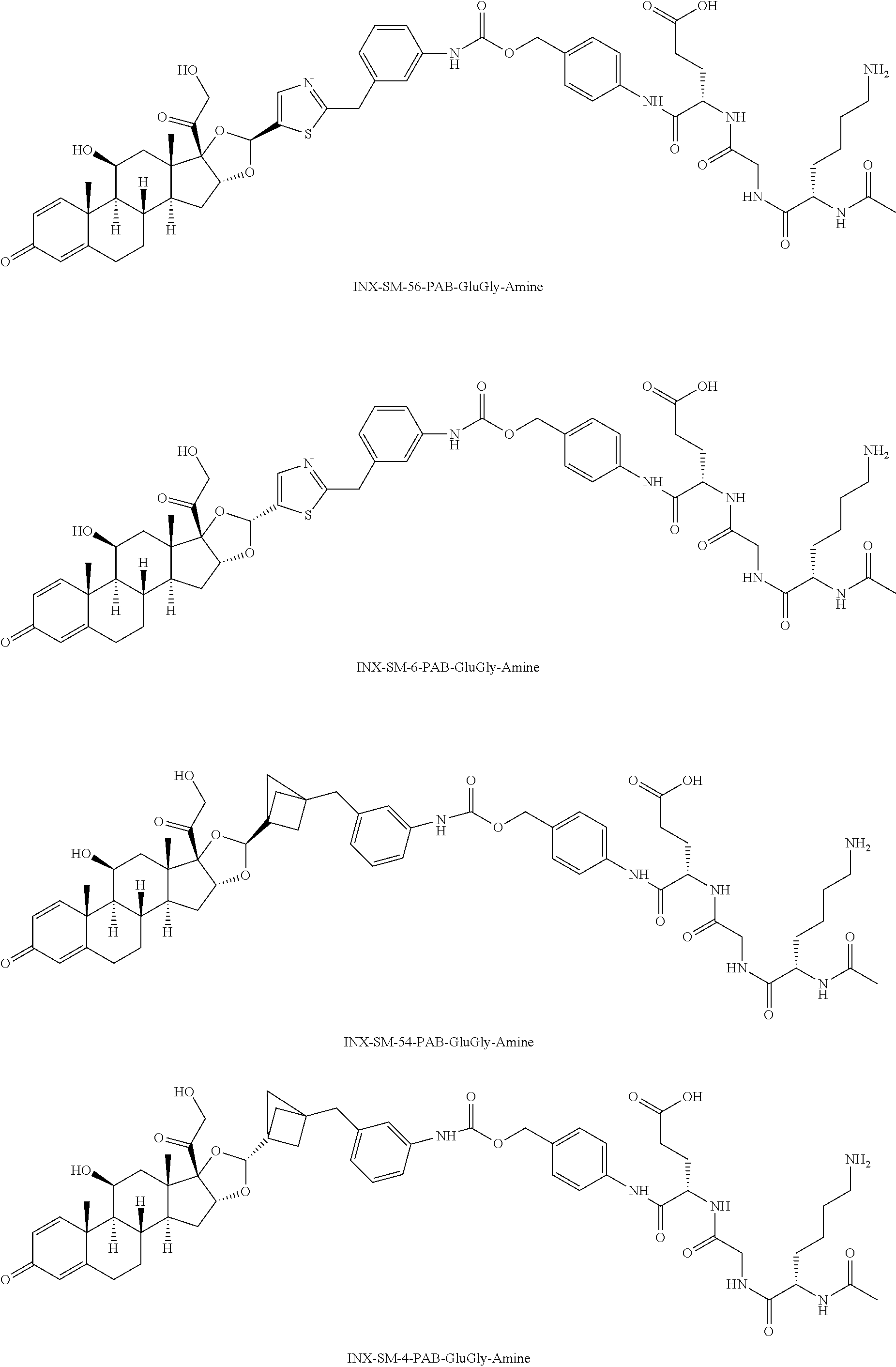

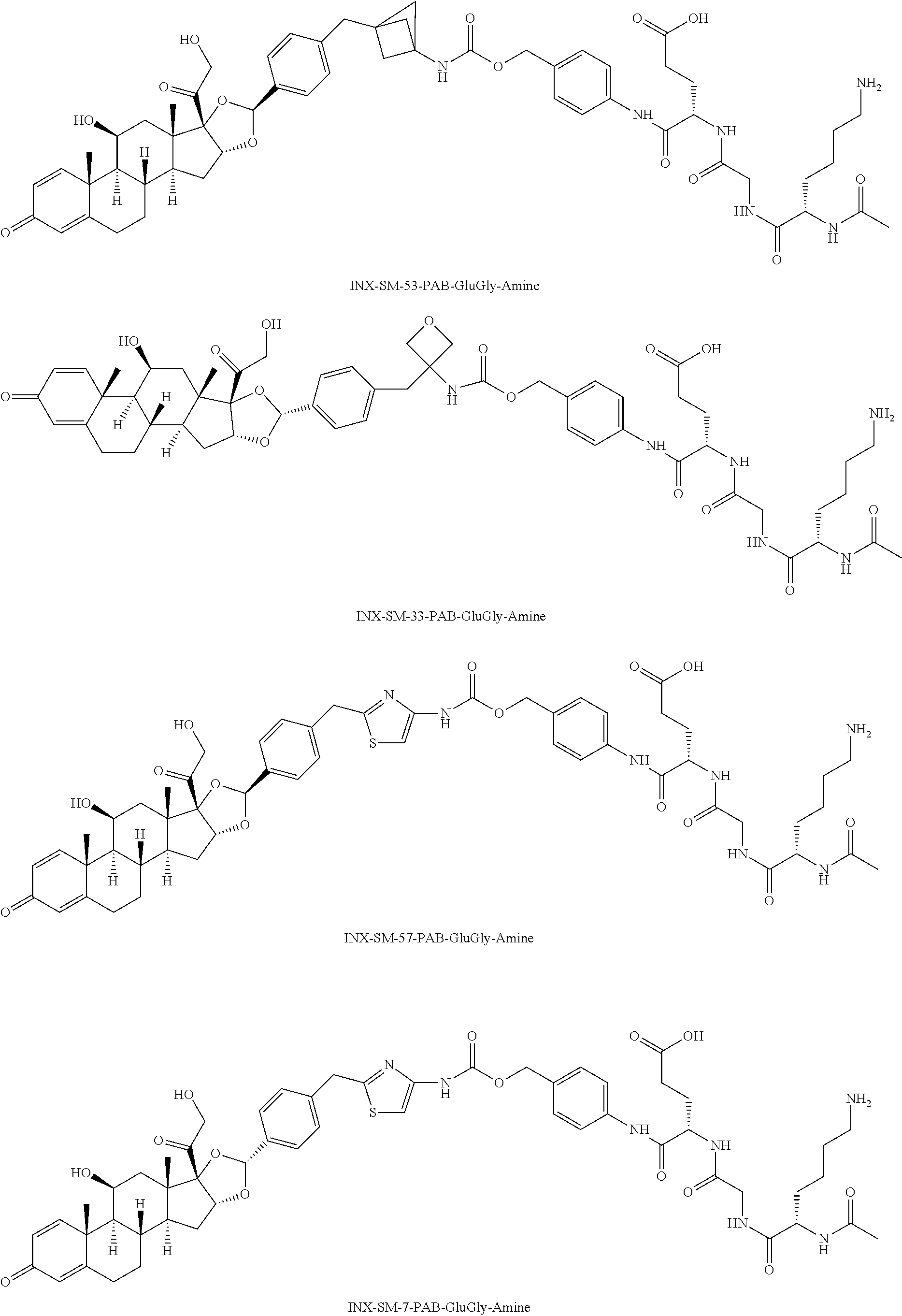

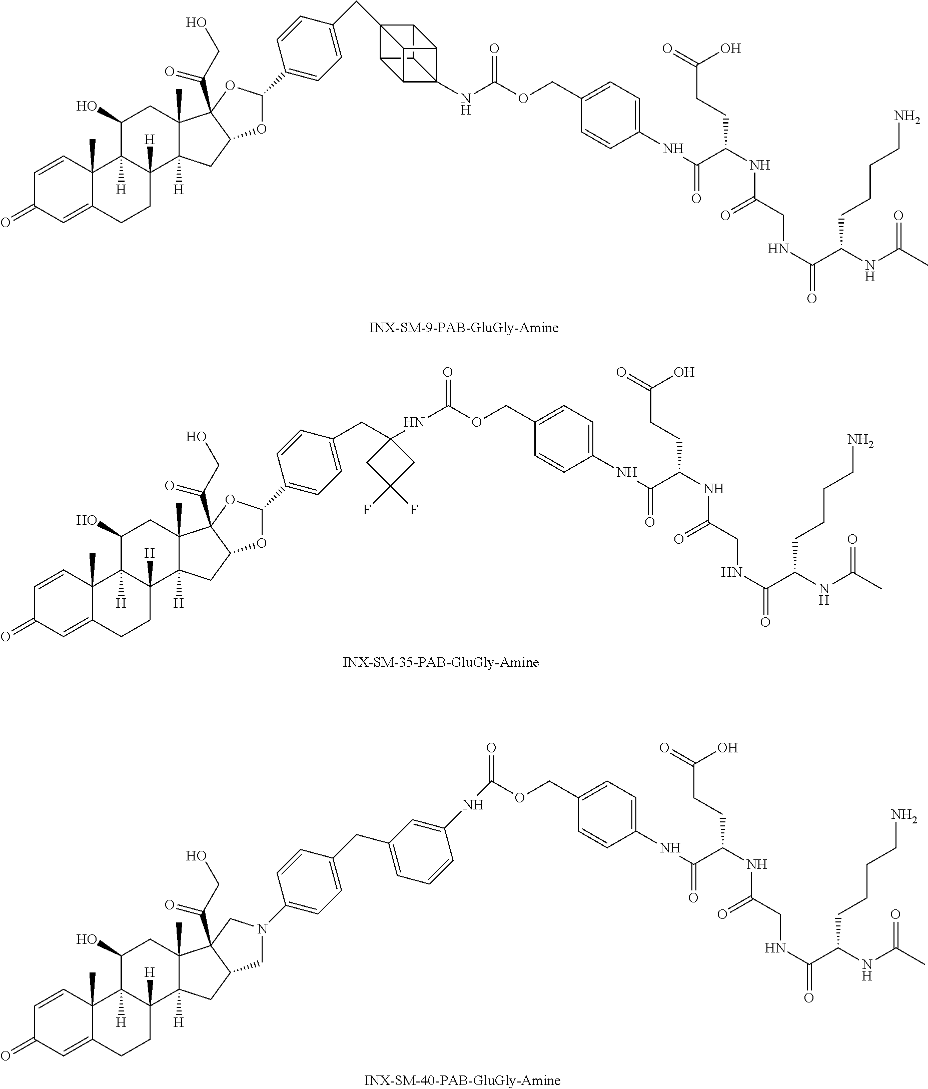

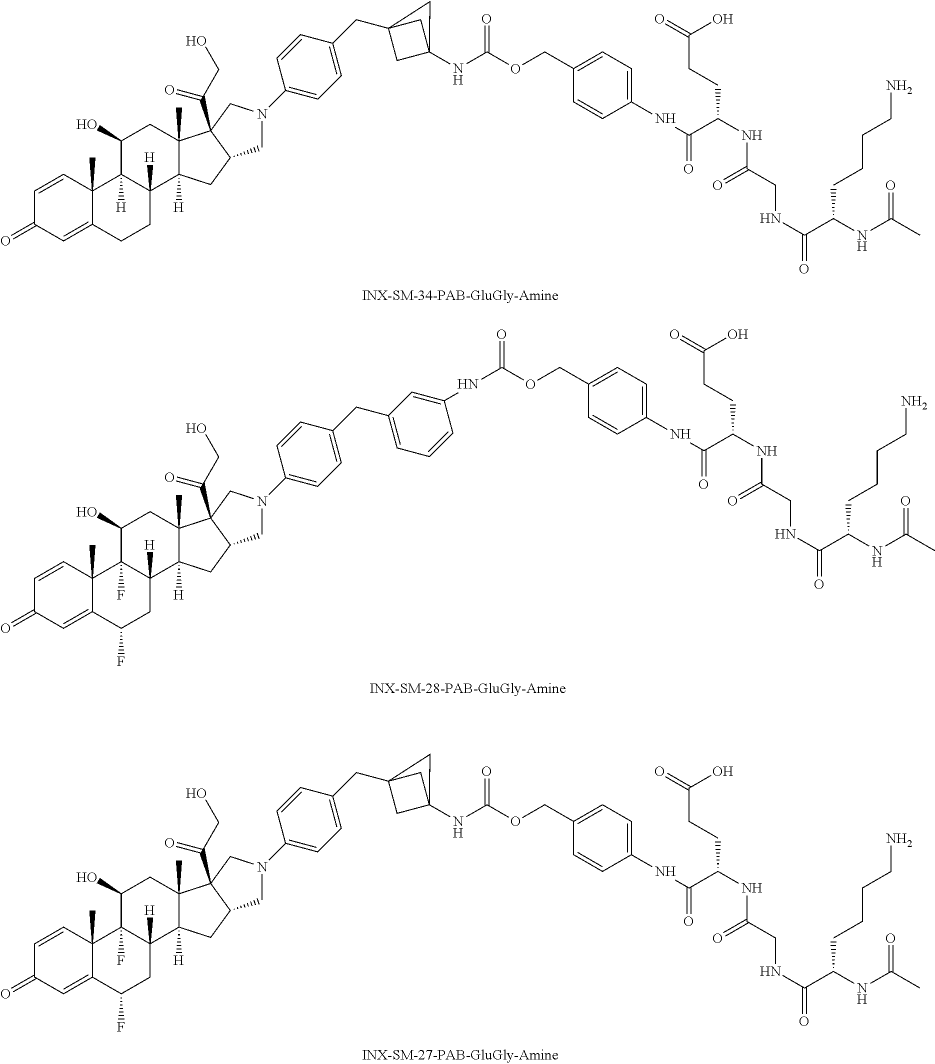

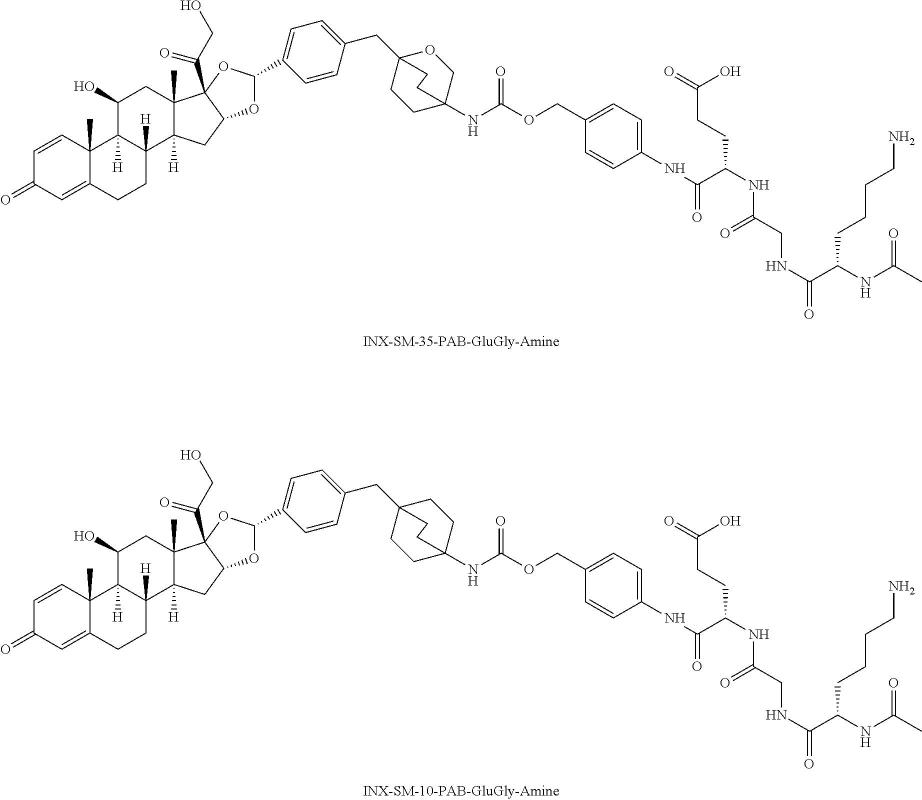

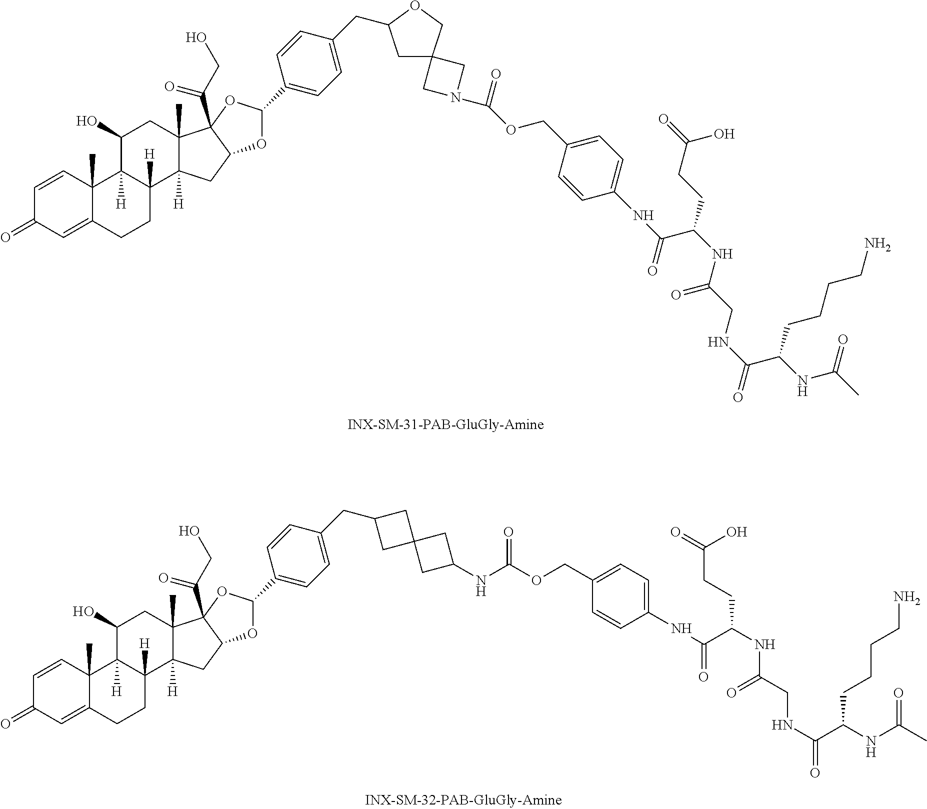

### (II) Payload-Linker-J

Where,
Linker=Glucuronidase cleavable sugar (GlcA)+Immolative linker Para Amino Benzyl
J=Alkoxyamine

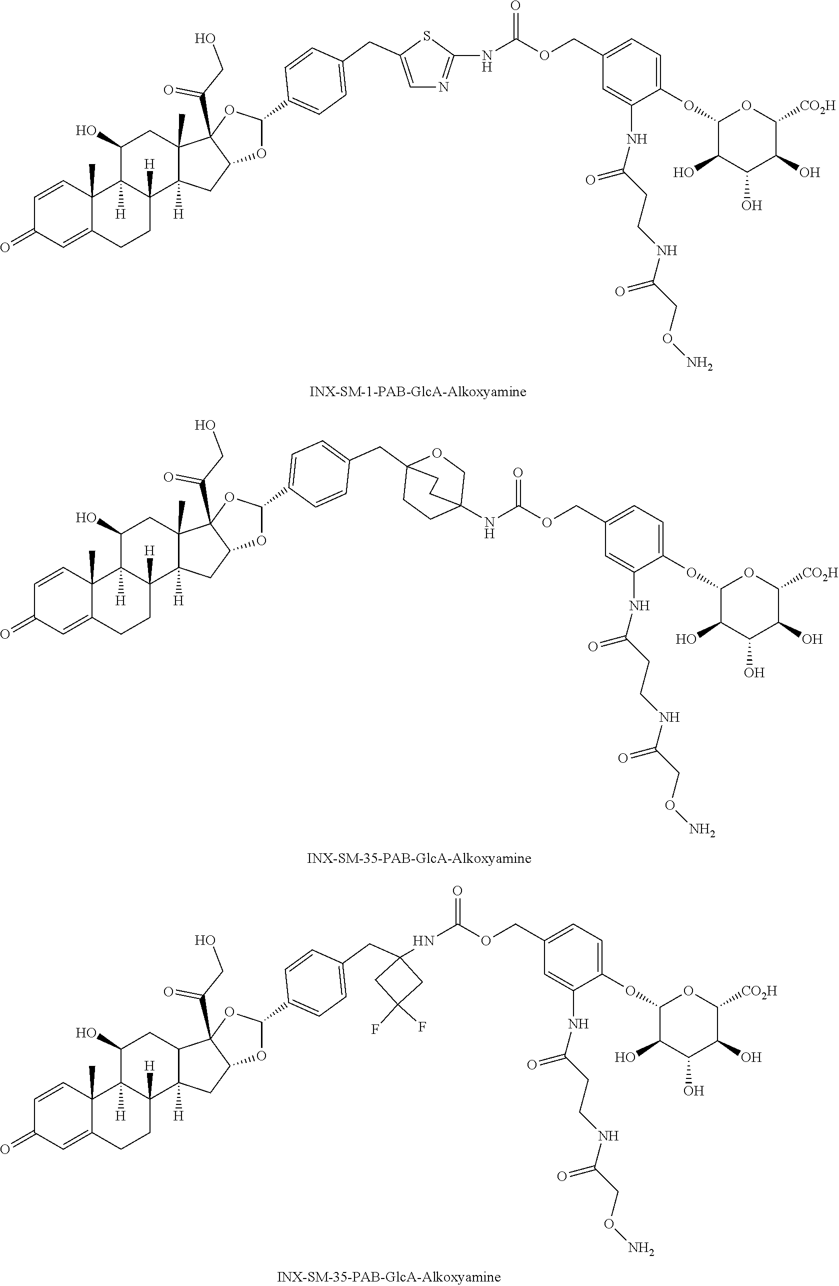

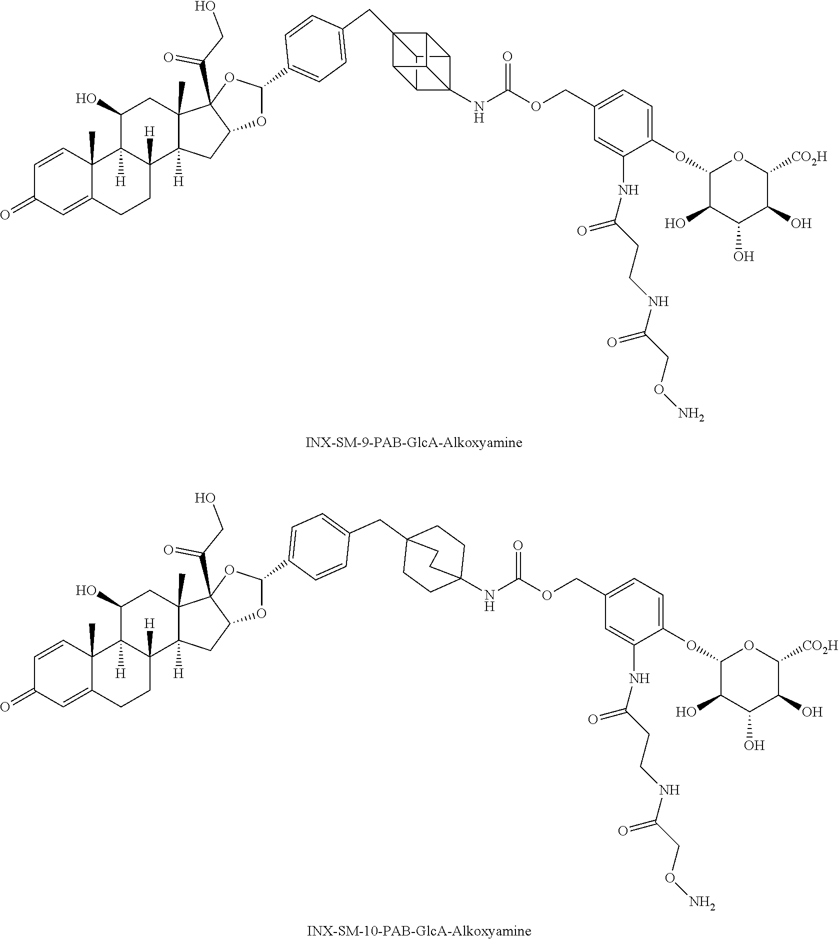

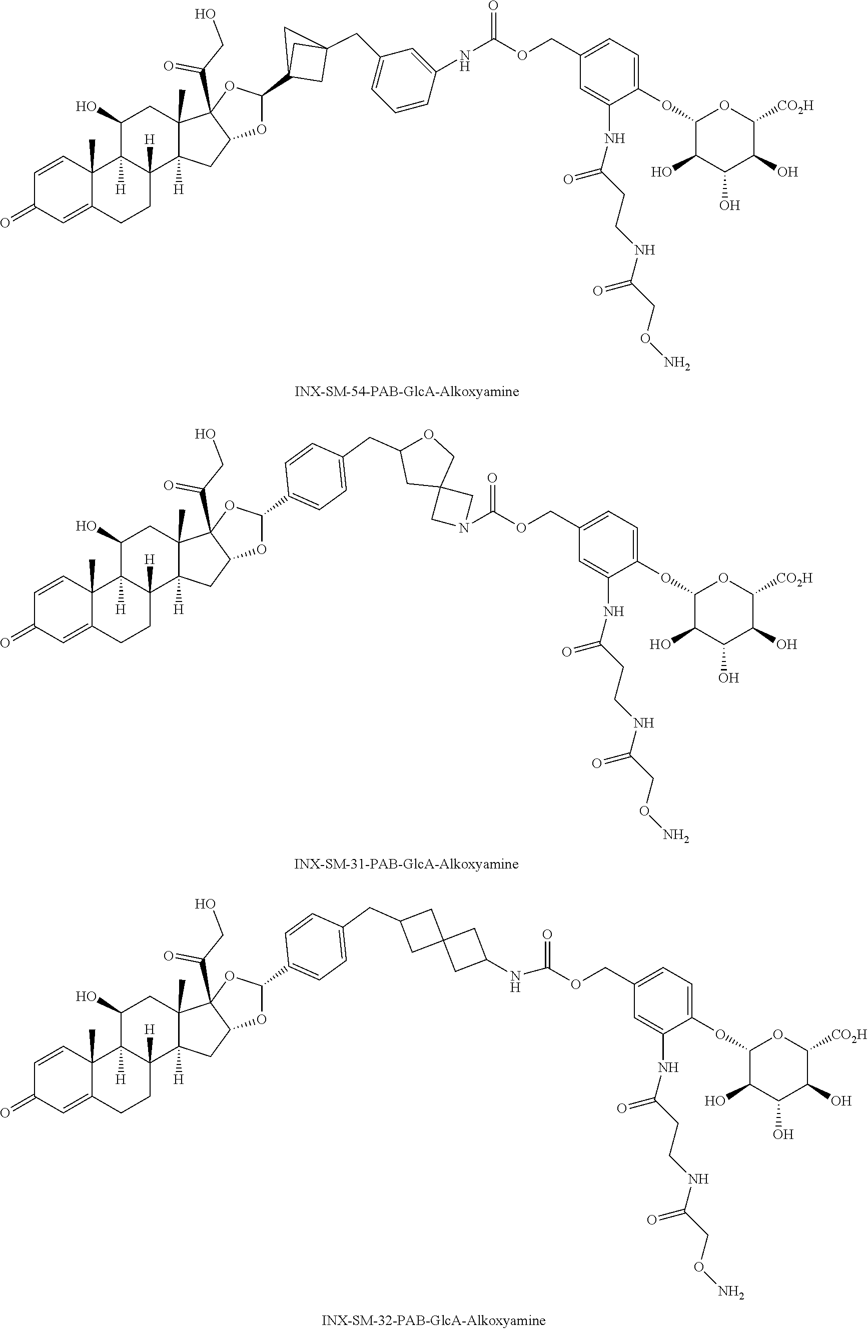

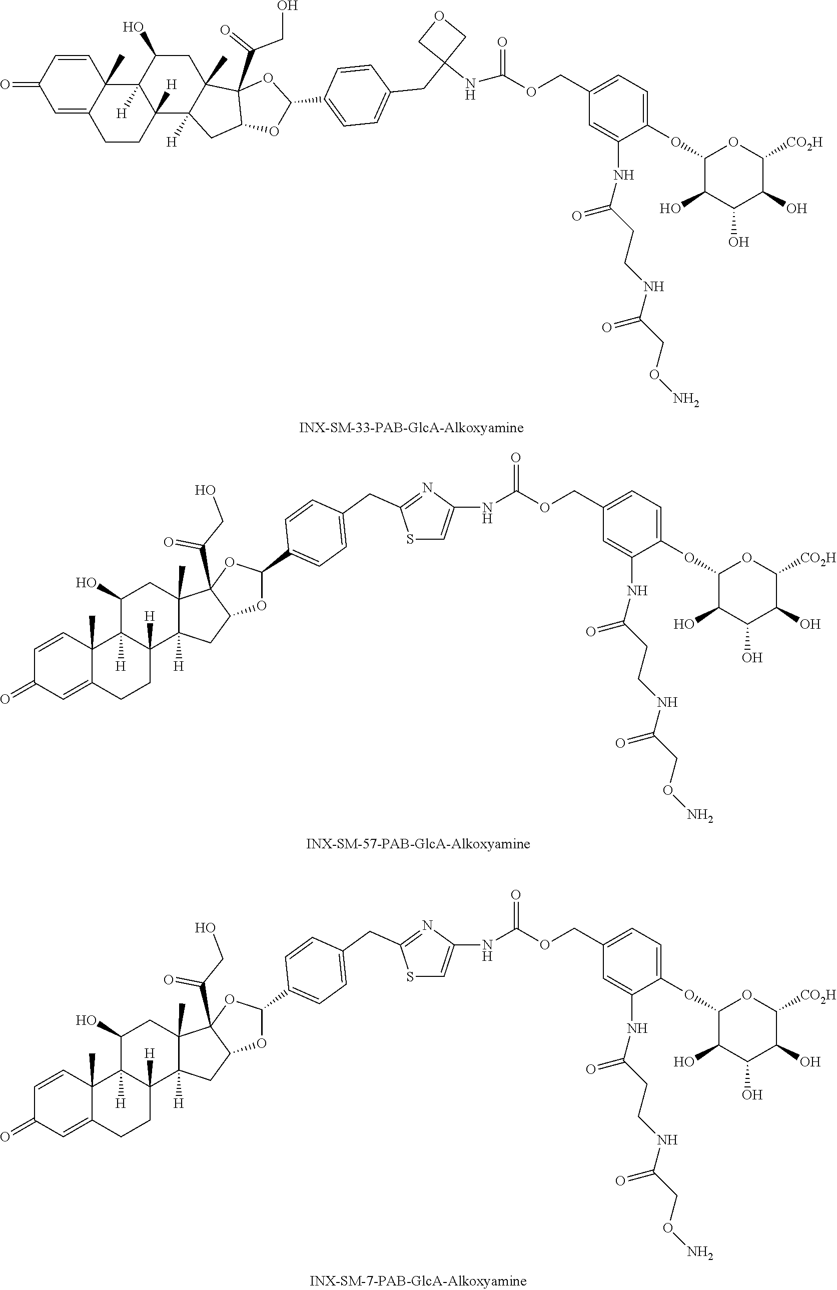

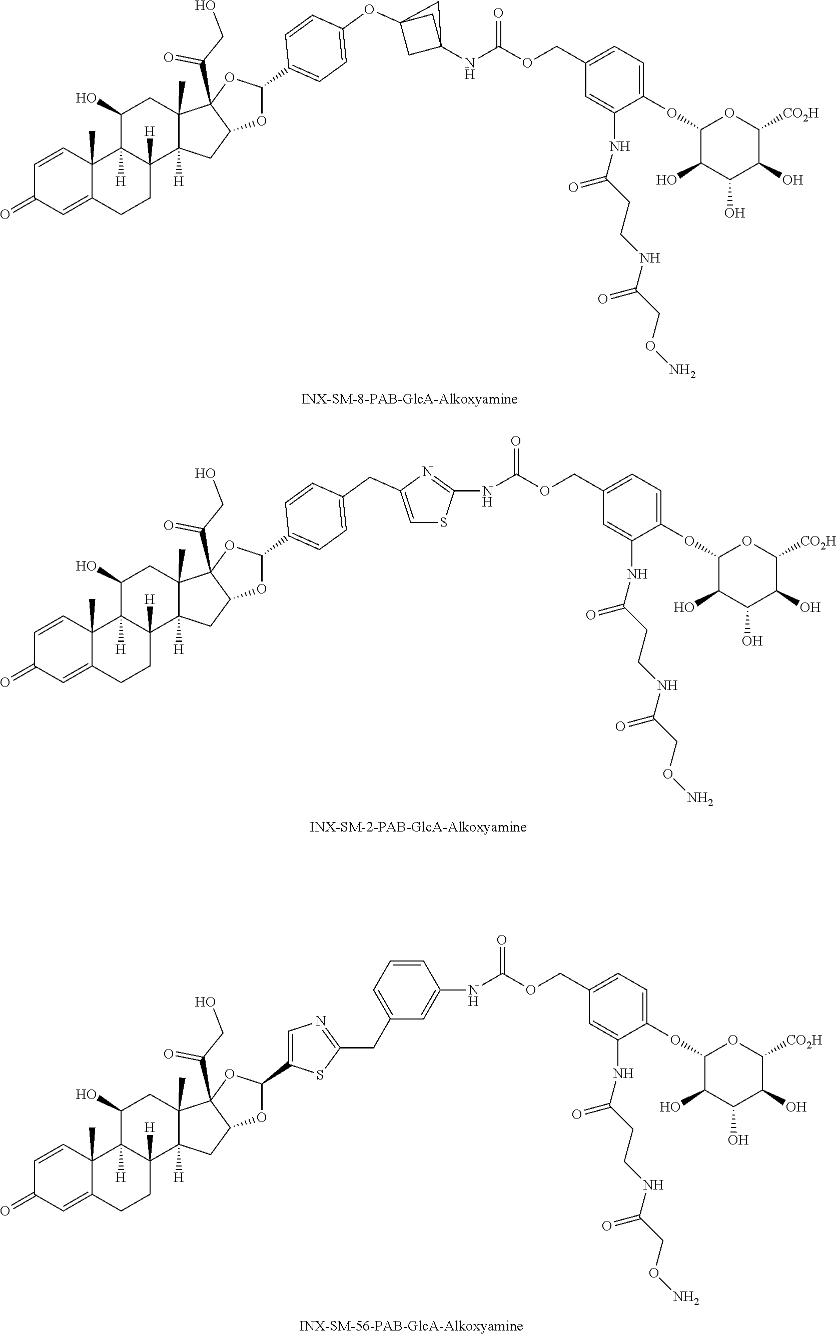

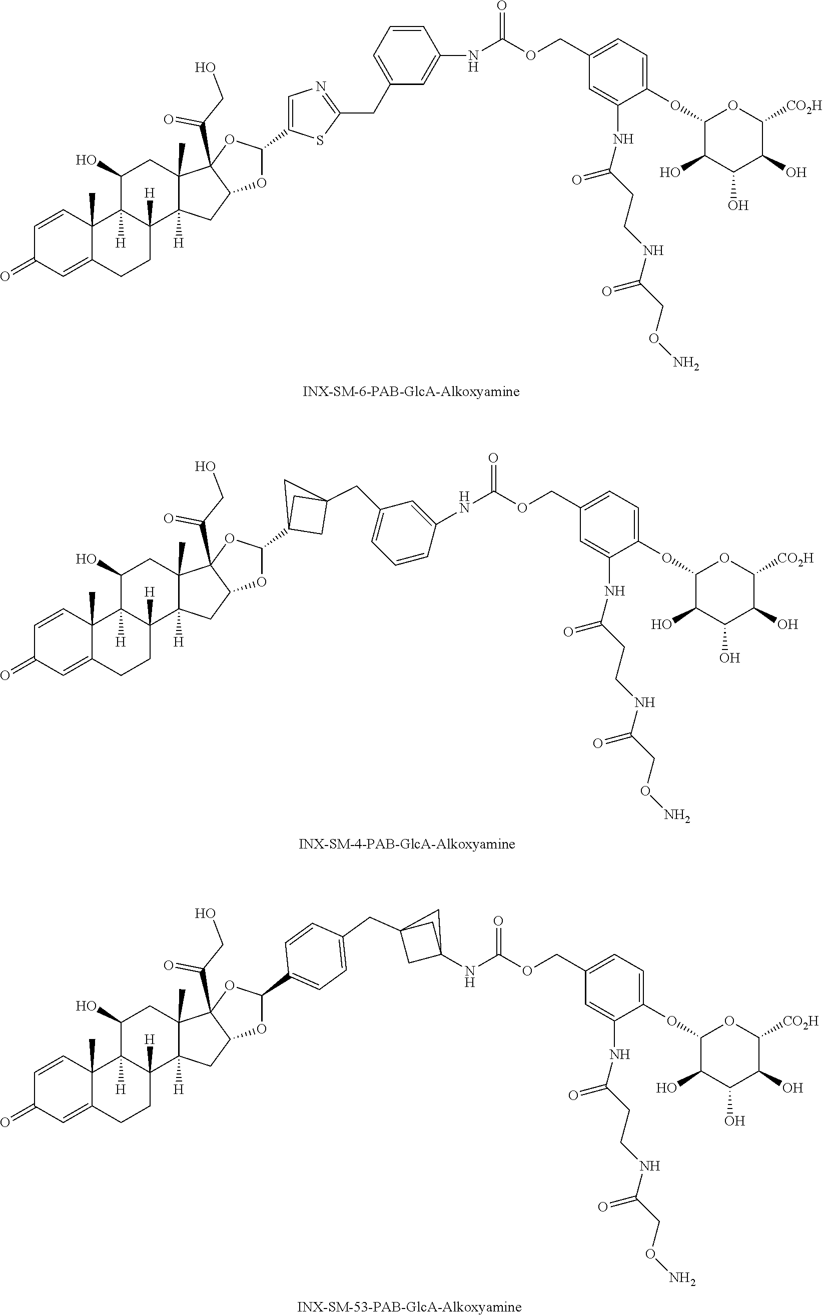

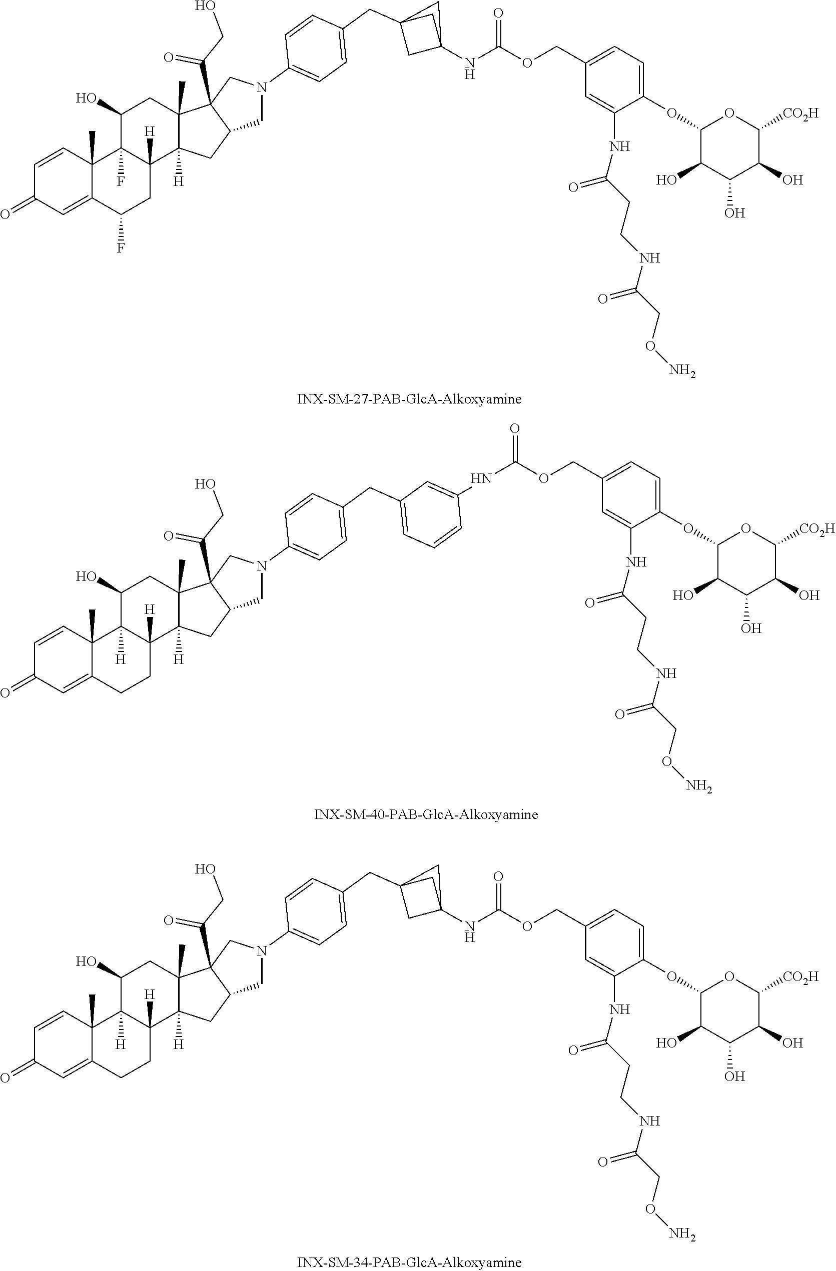

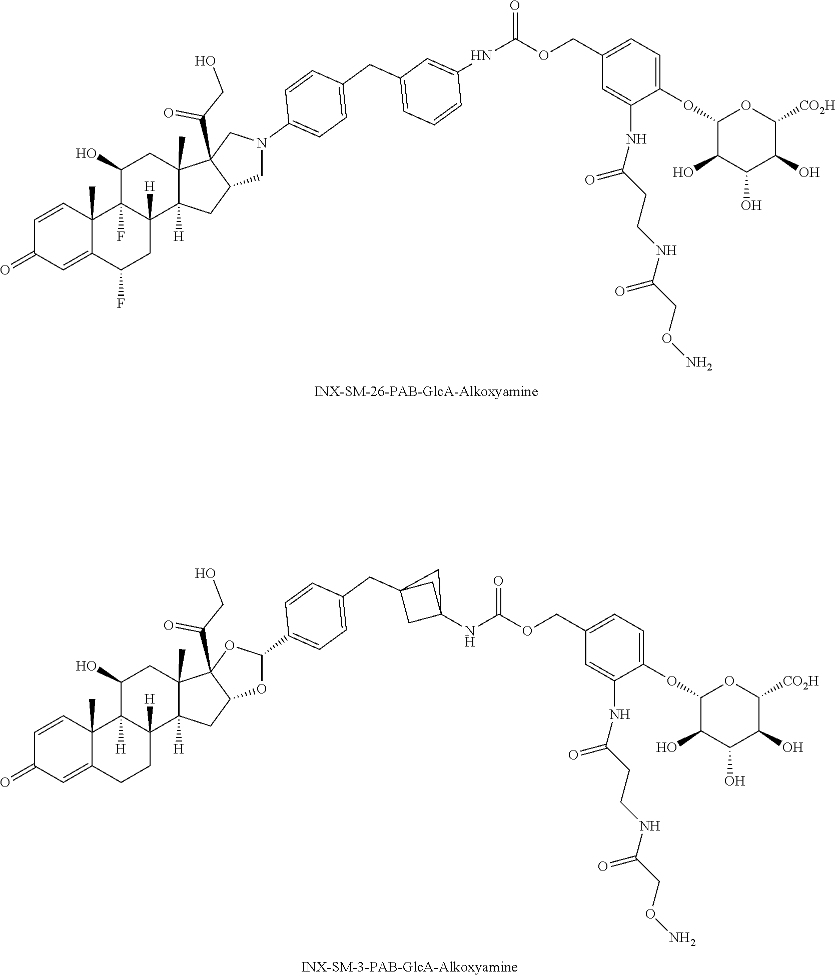

Where,
Linker=Glucuronidase cleavable sugar (GlcA)+Immolative linker Para Amino Benzyl (PAB)
J=Bromoacetyl

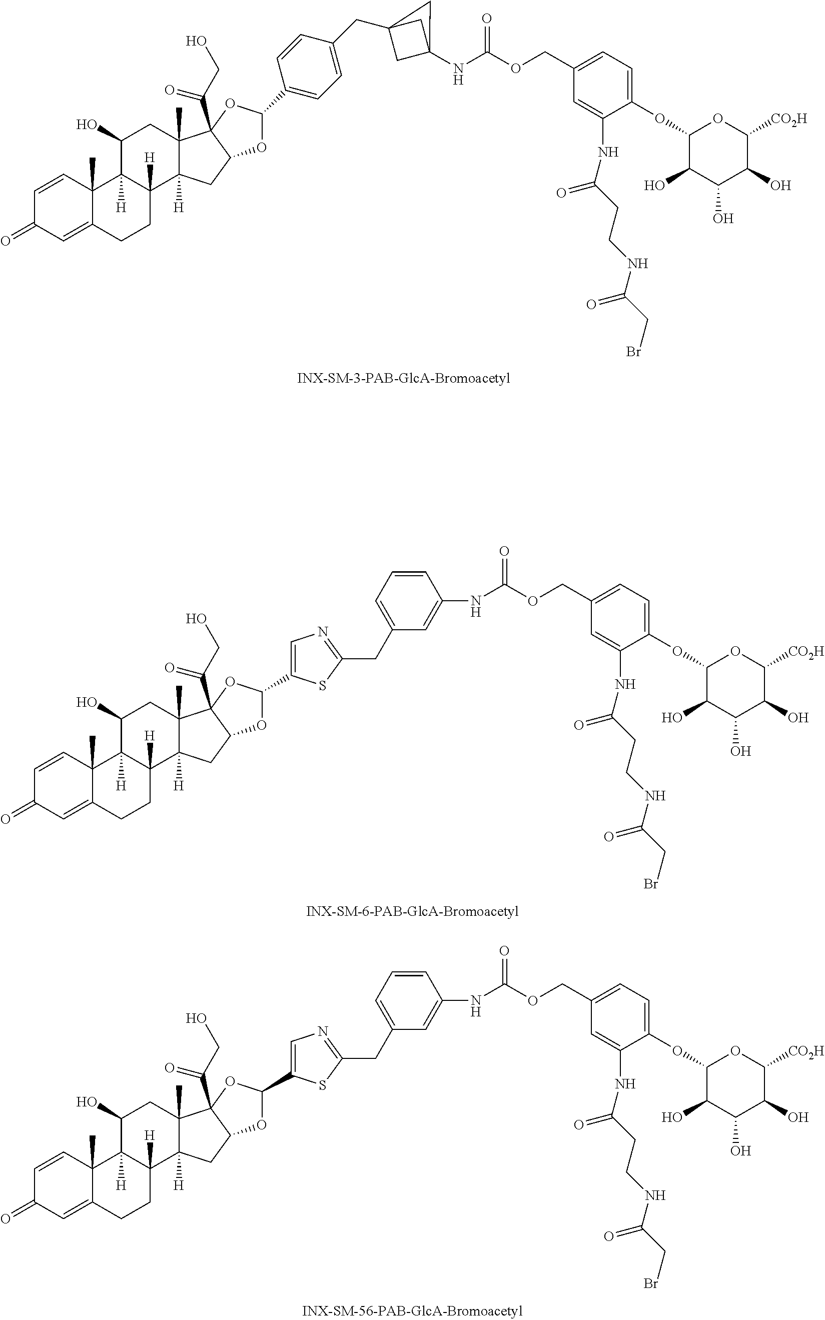

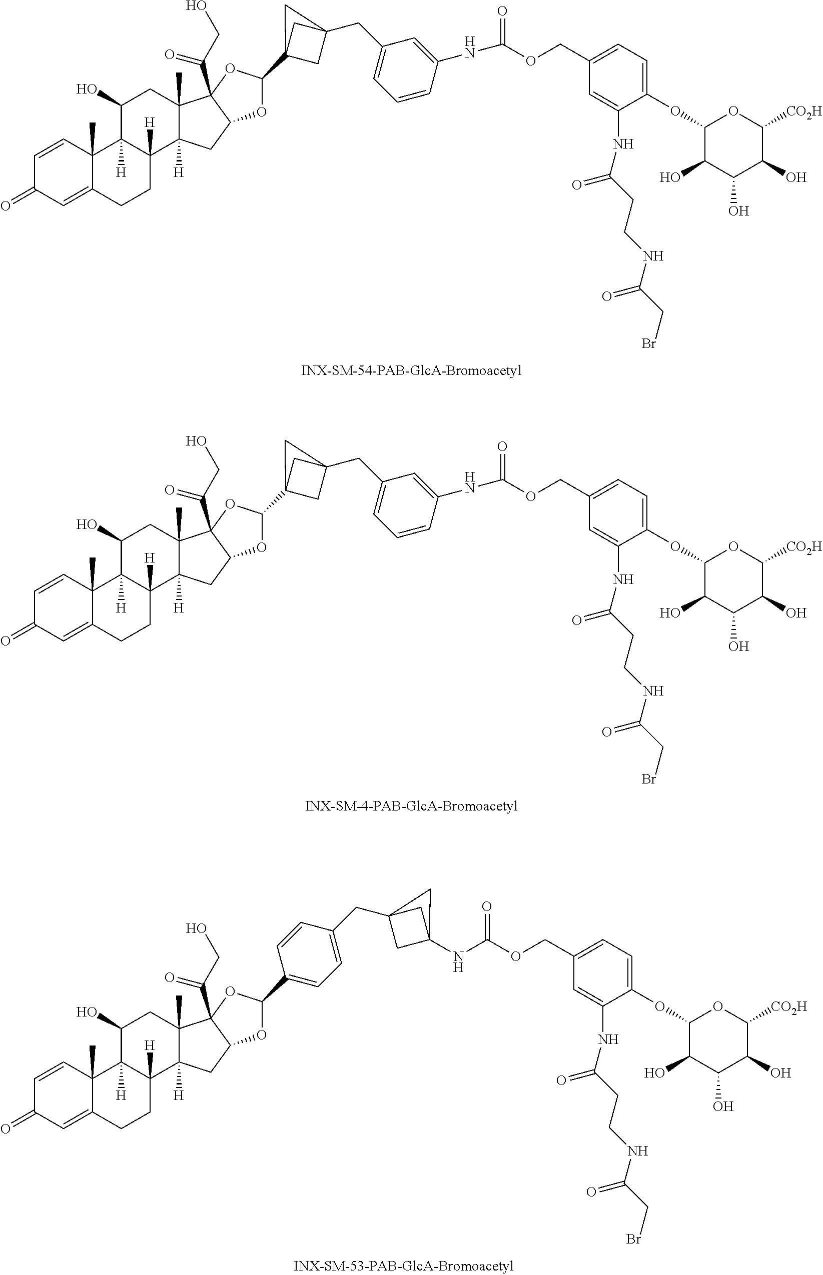

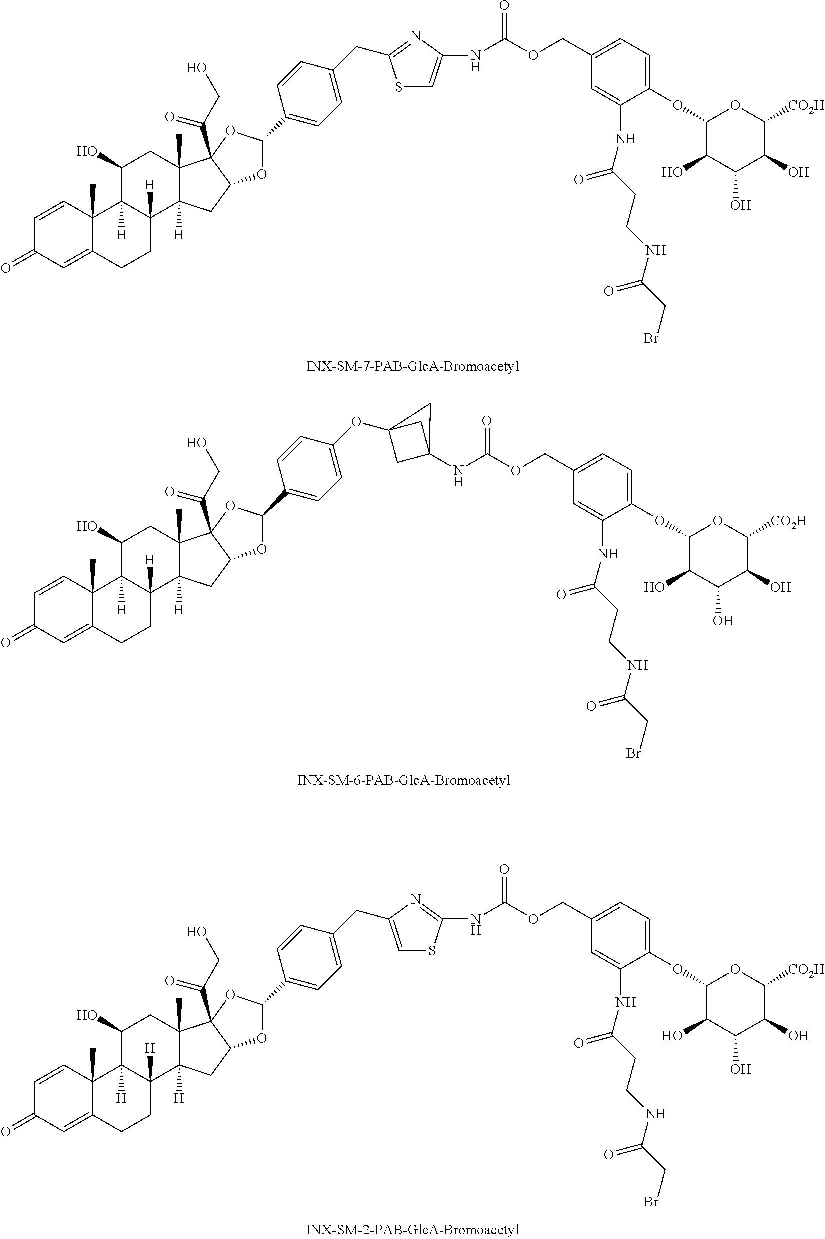

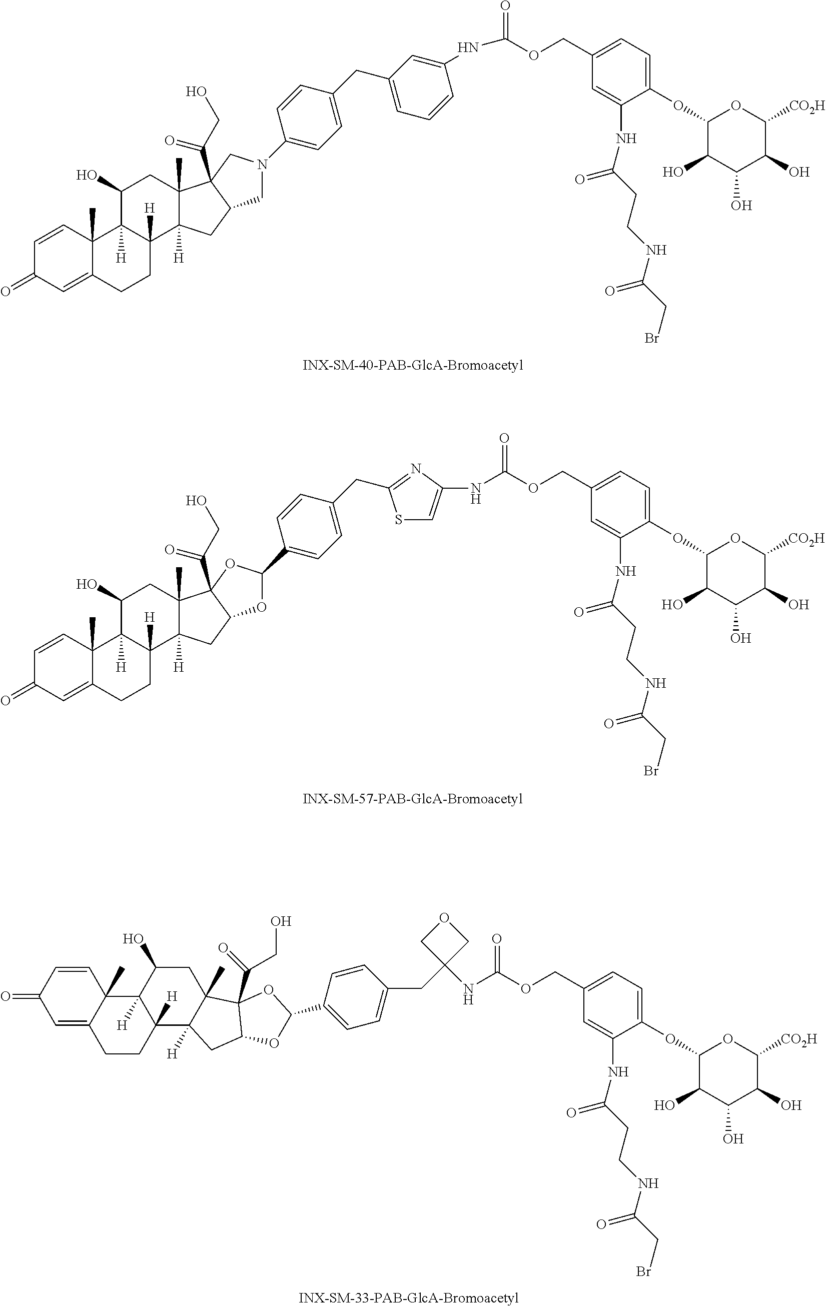

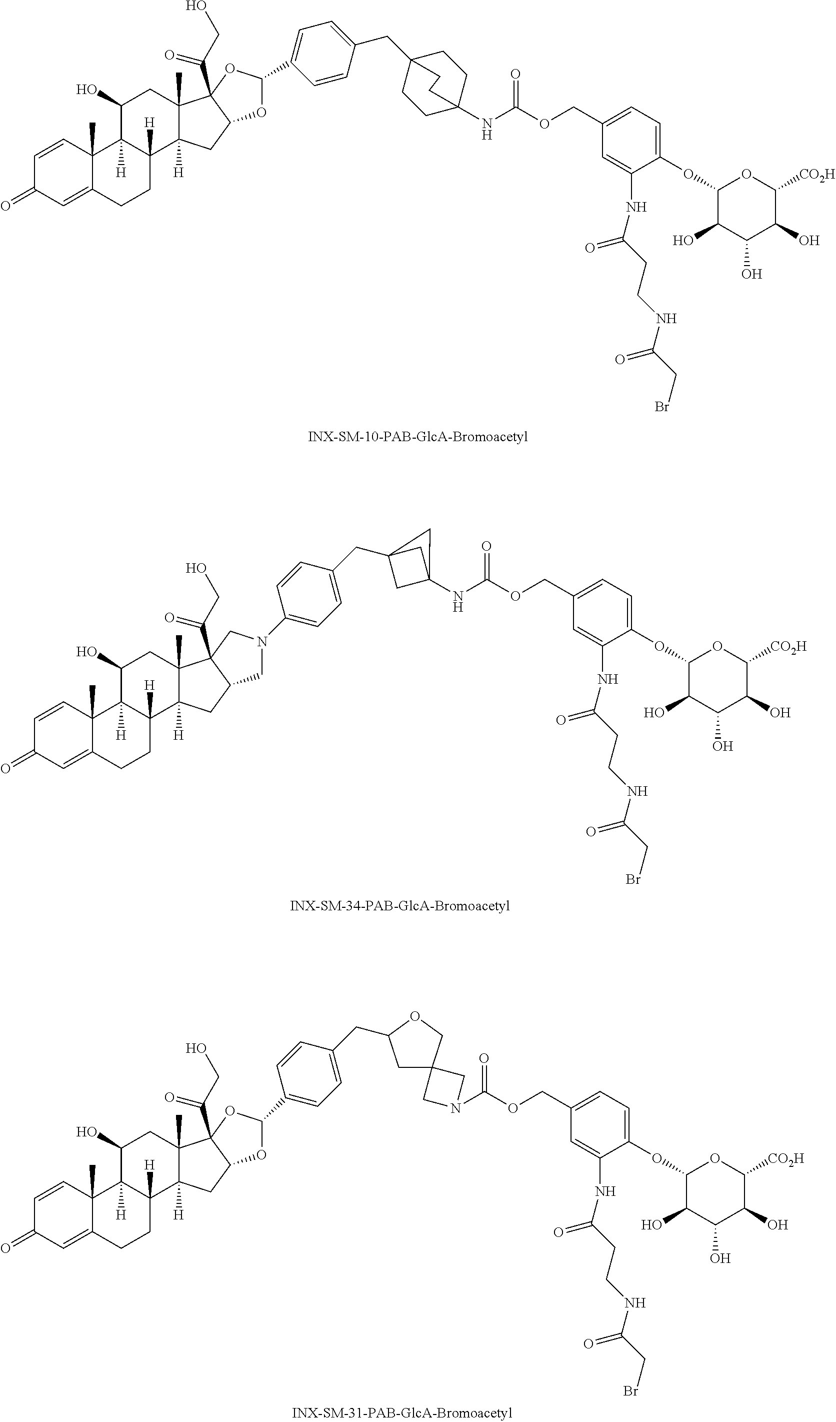

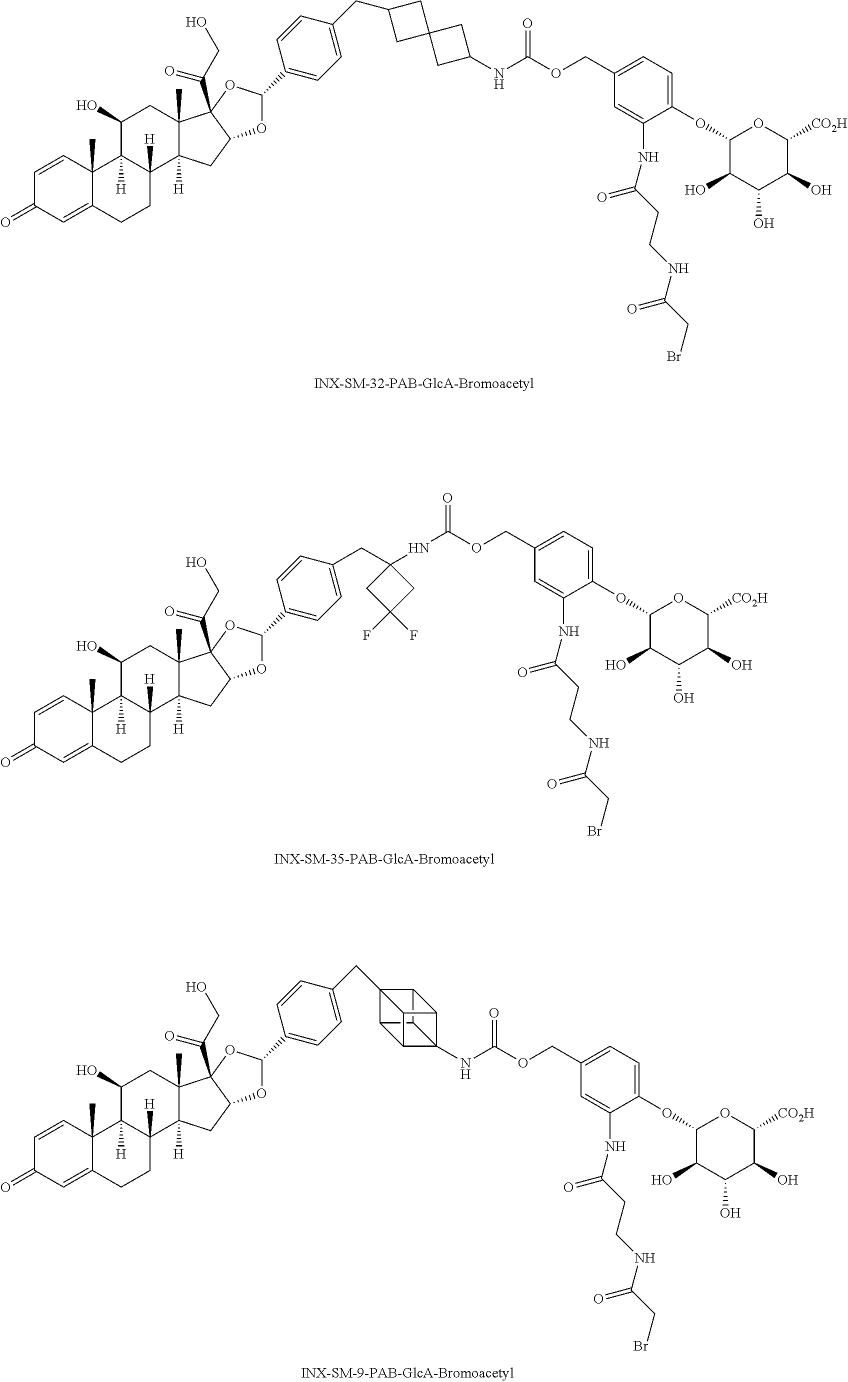

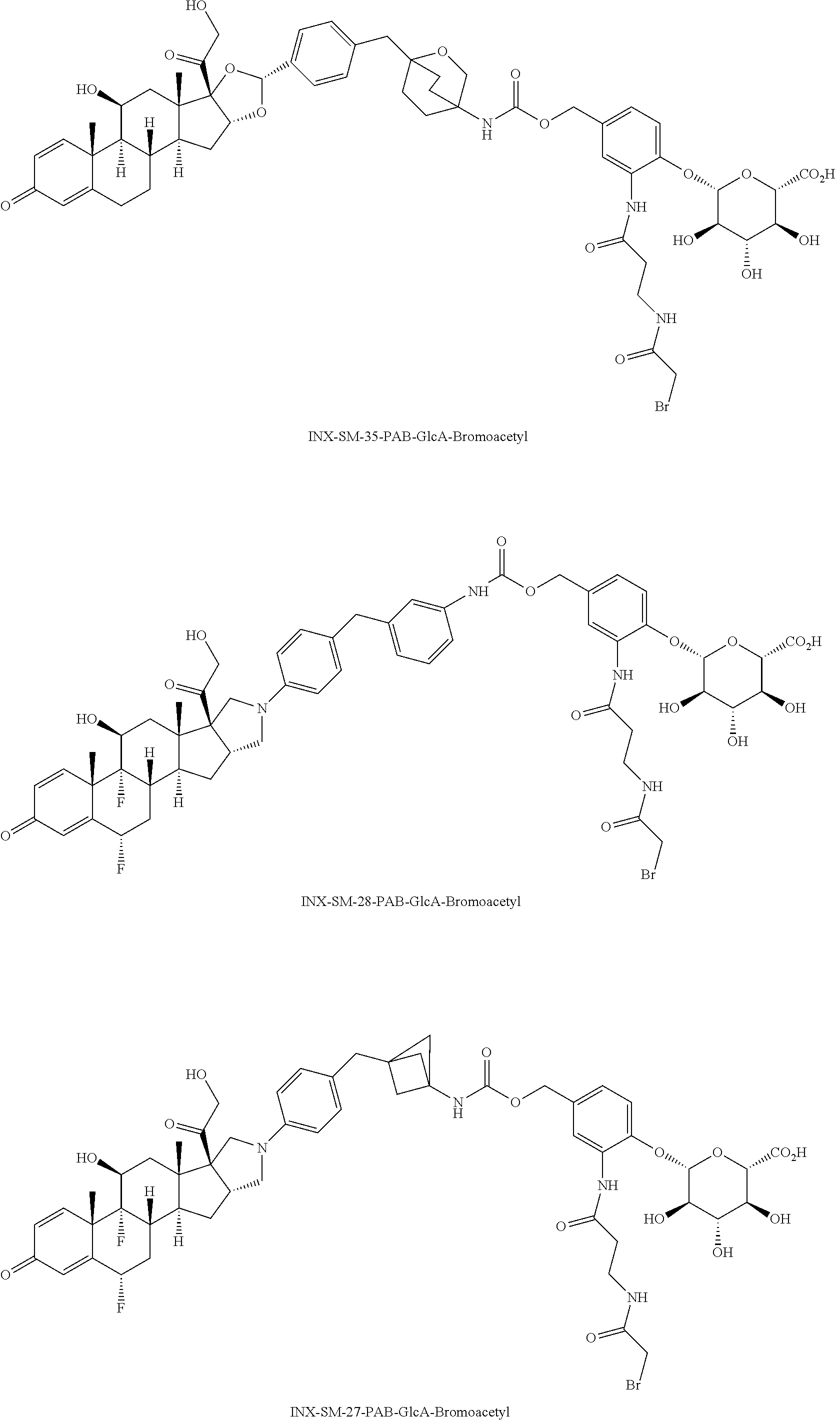

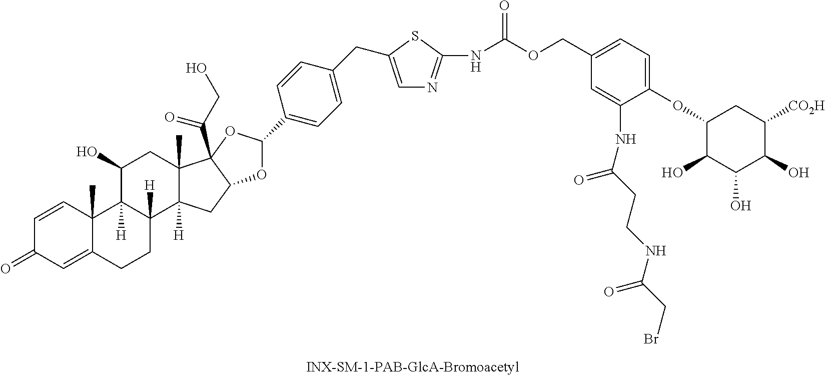

Where,
Linker=Glucuronidase cleavable sugar (GlcA)+Immolative linker Para Amino Benzyl (PAB)
J=Dibenzocyclooctyne

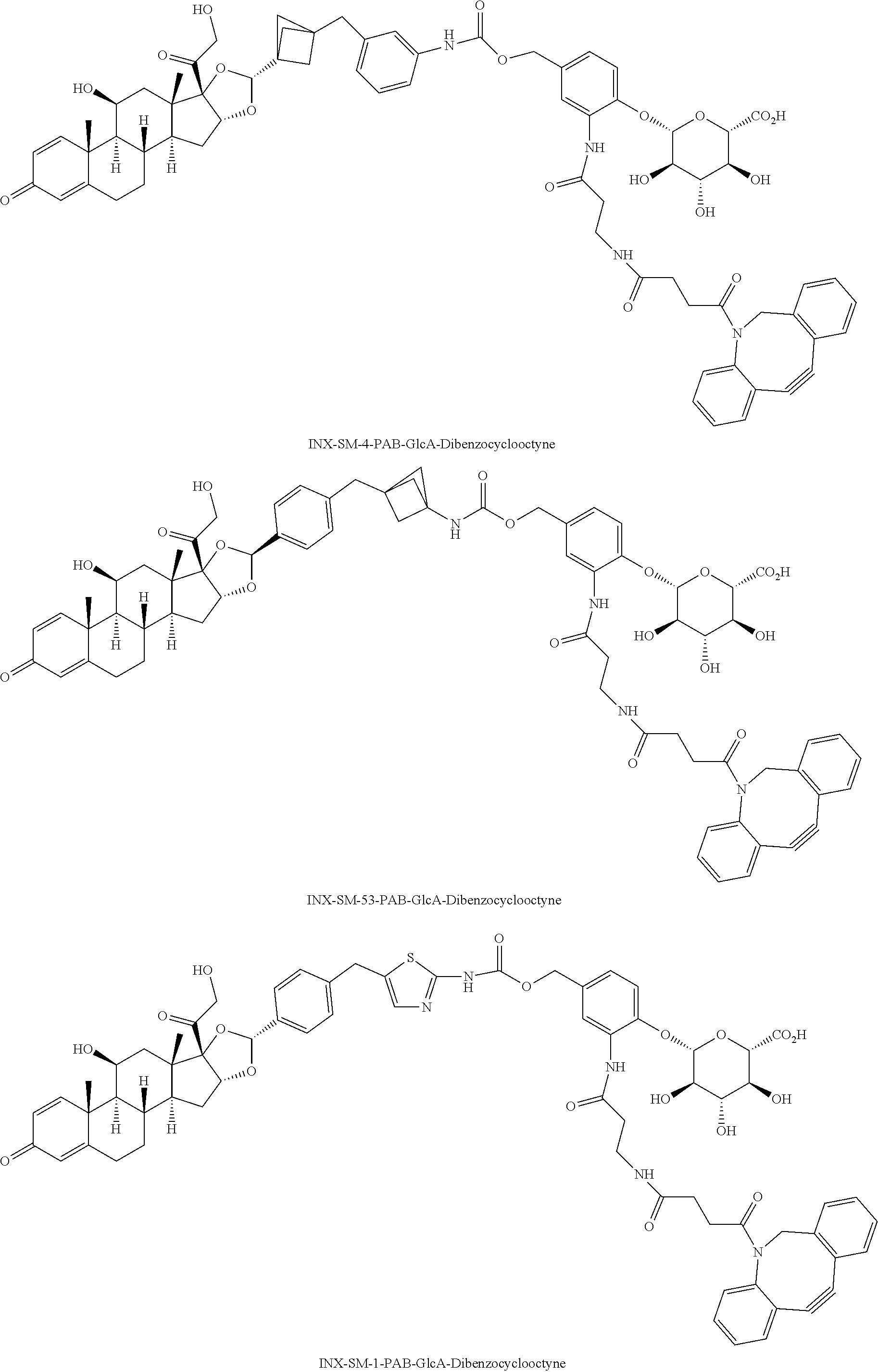

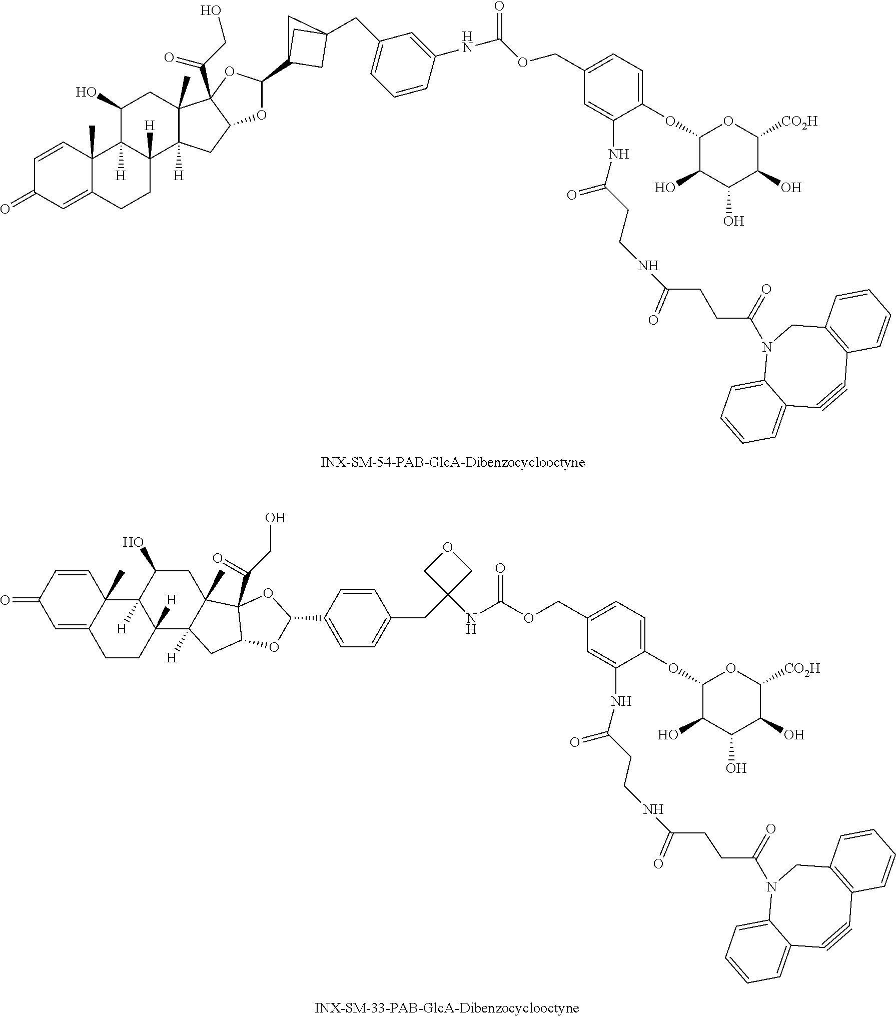

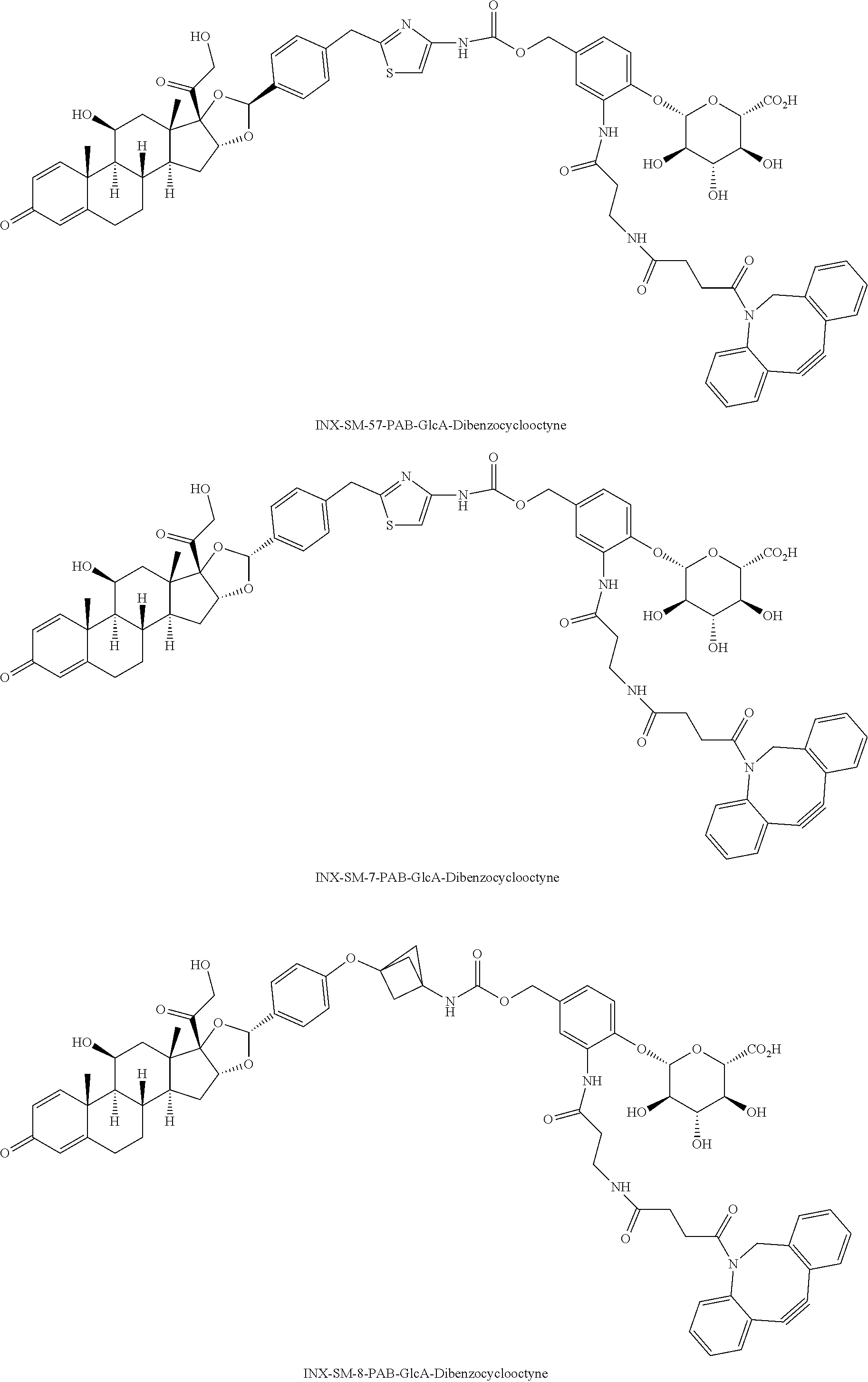

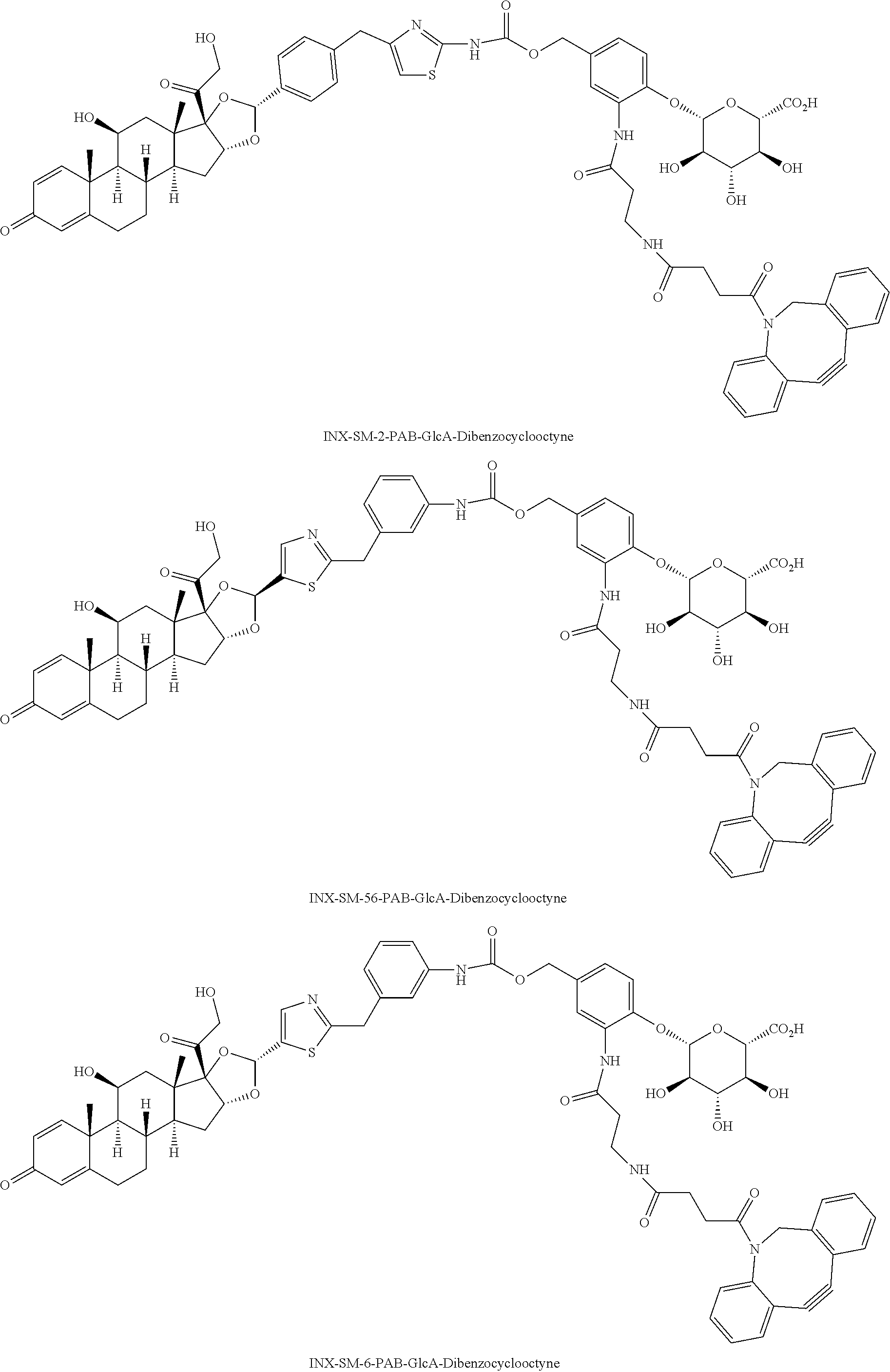

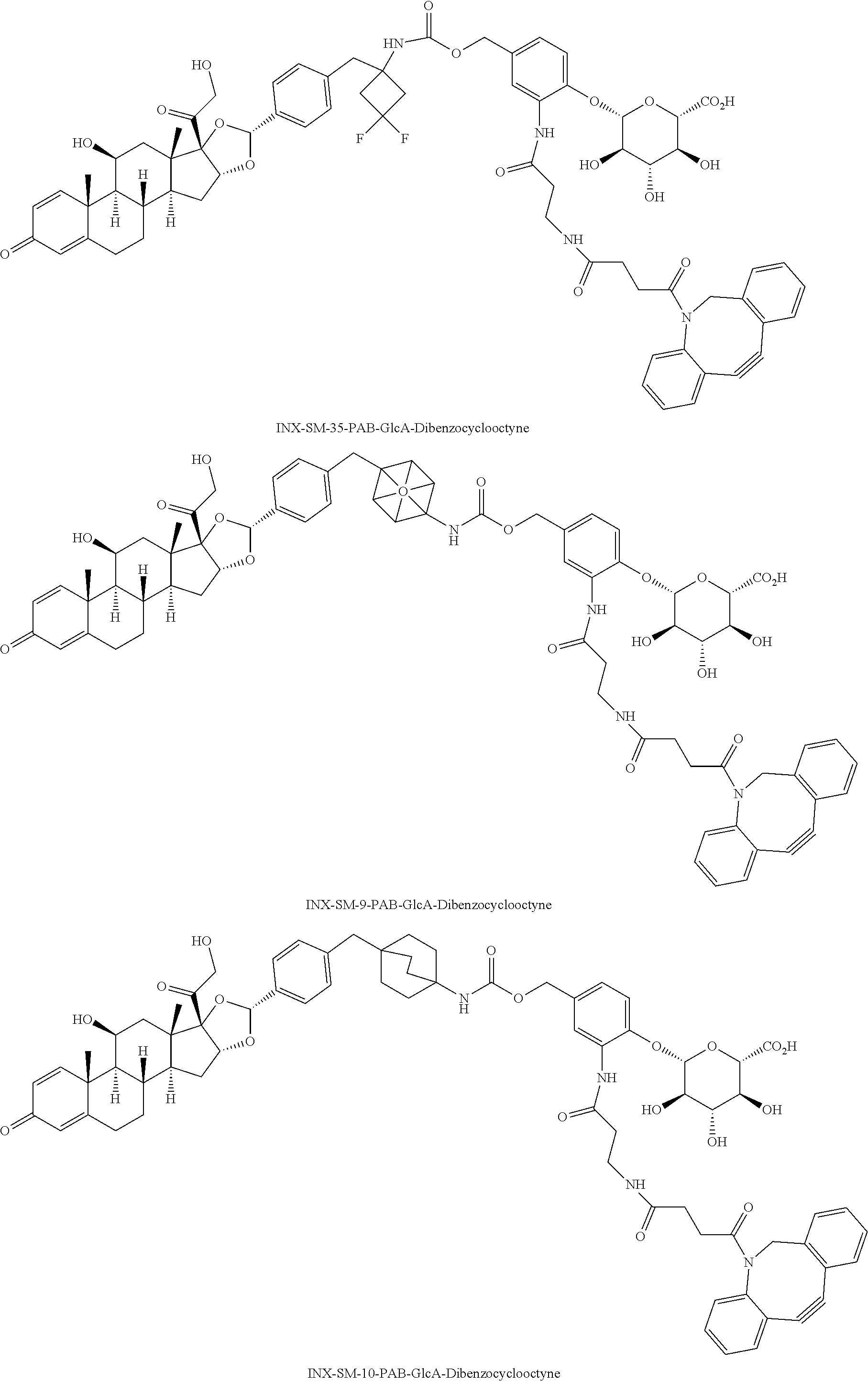

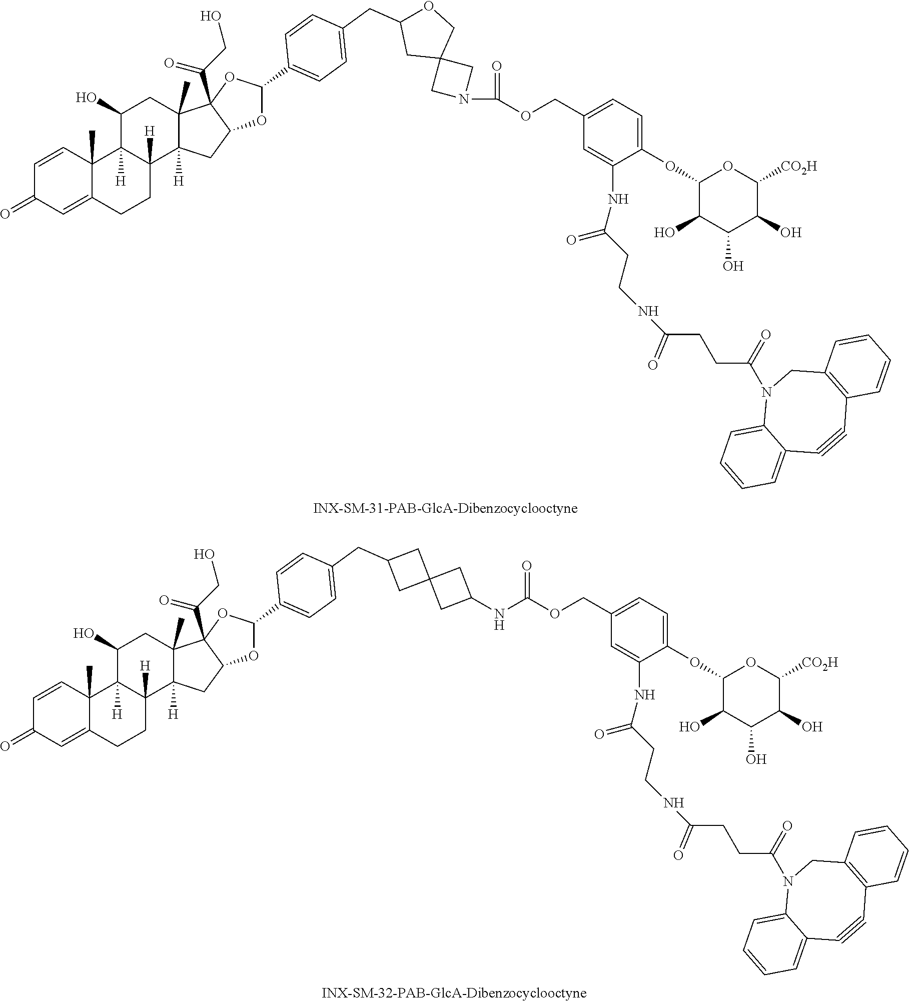

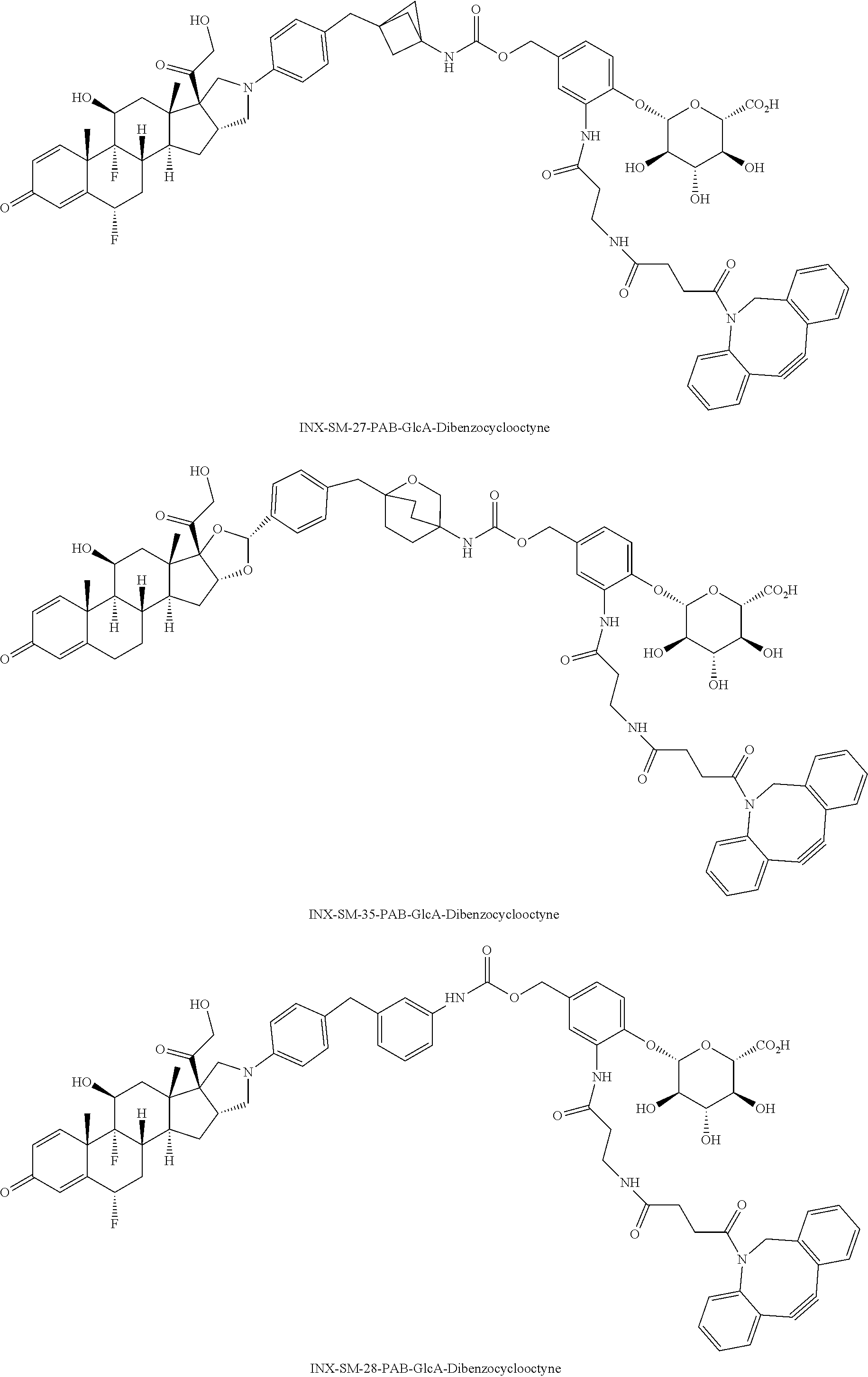

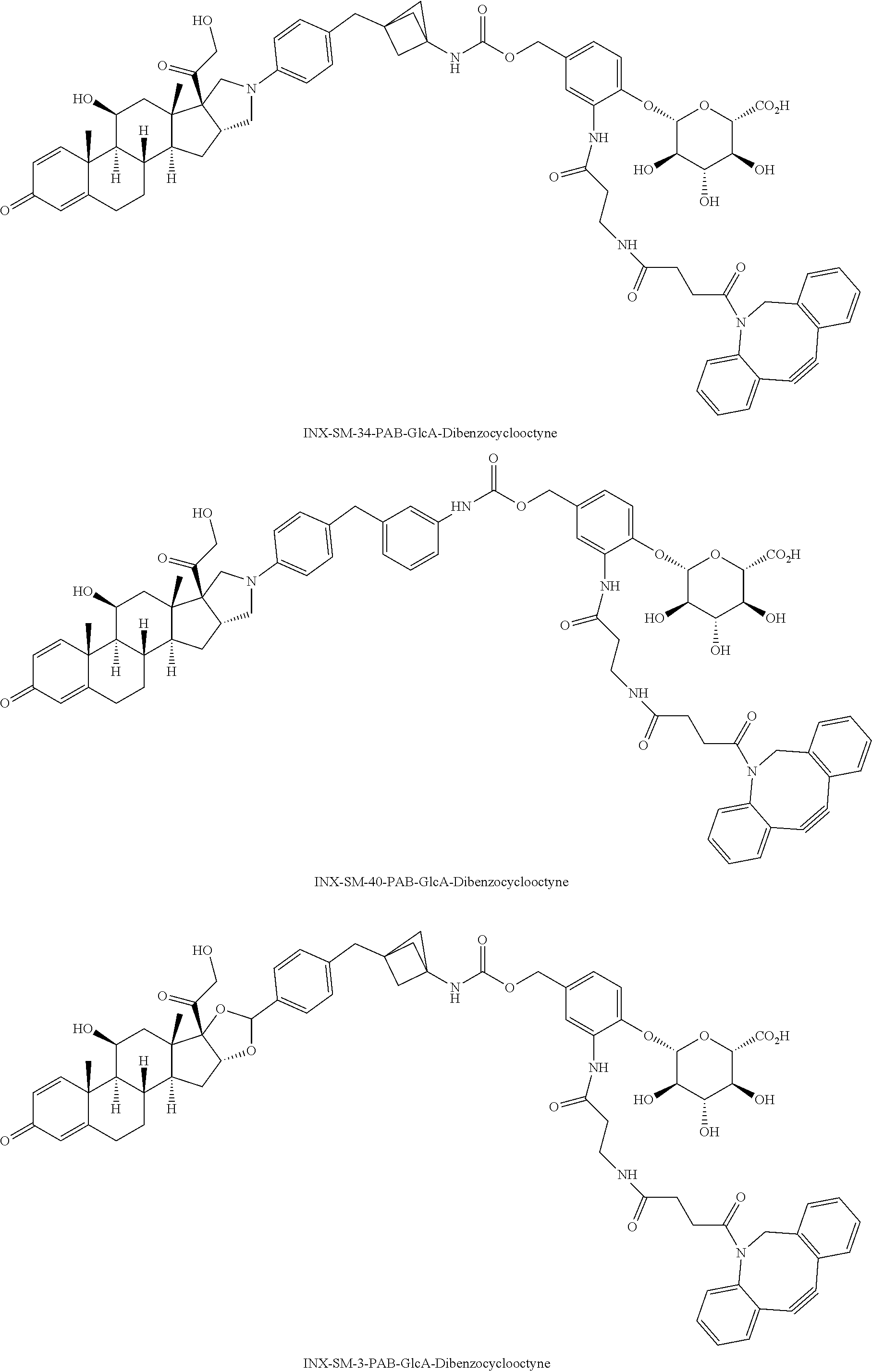

Where,
Linker=Glucuronidase cleavable sugar (GlcA)+Immolative linker Para Amino Benzyl (PAB)
J=Hydroxysuccinimide

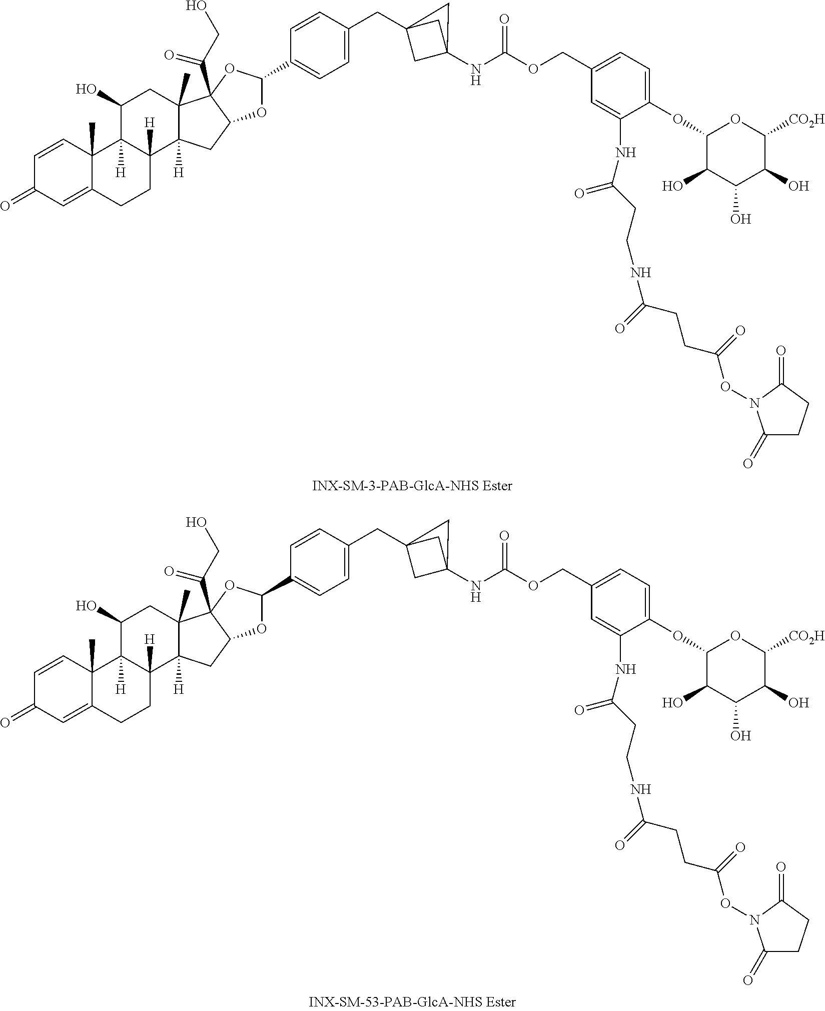

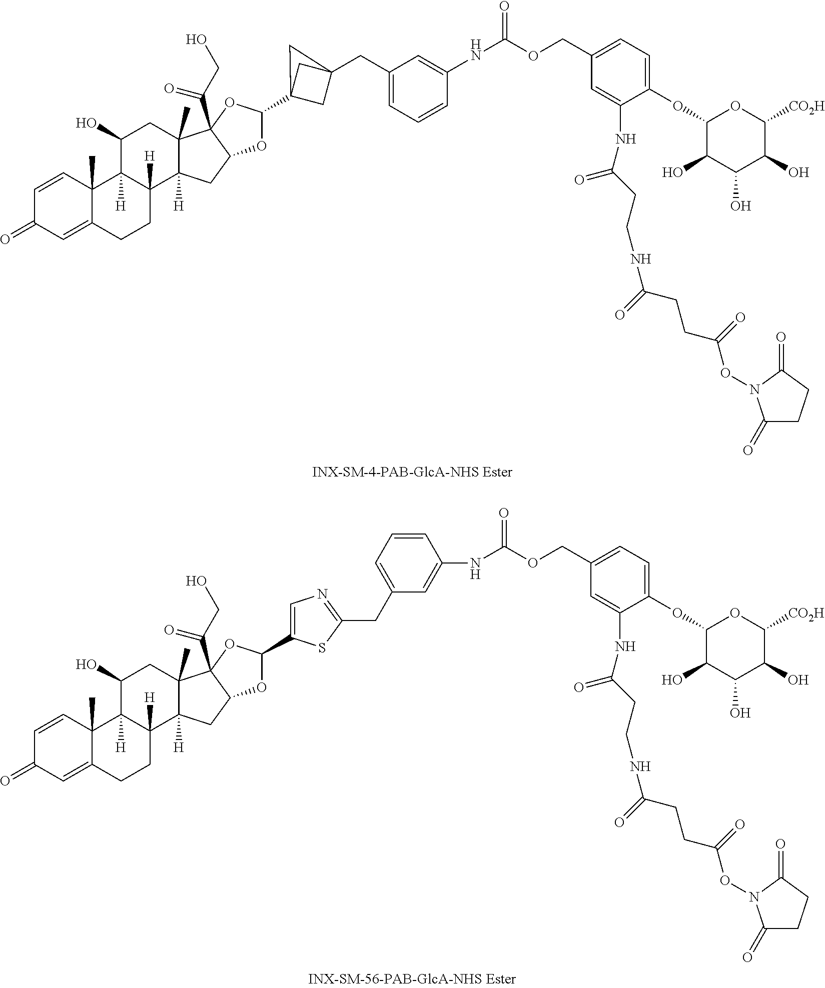

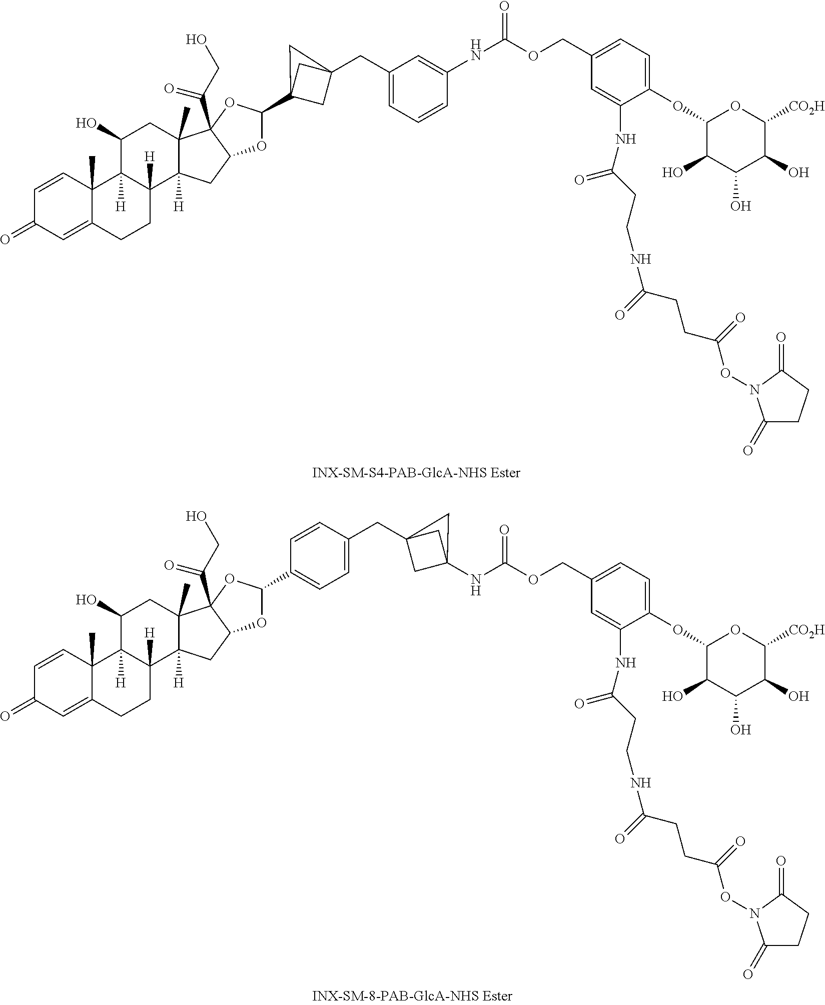

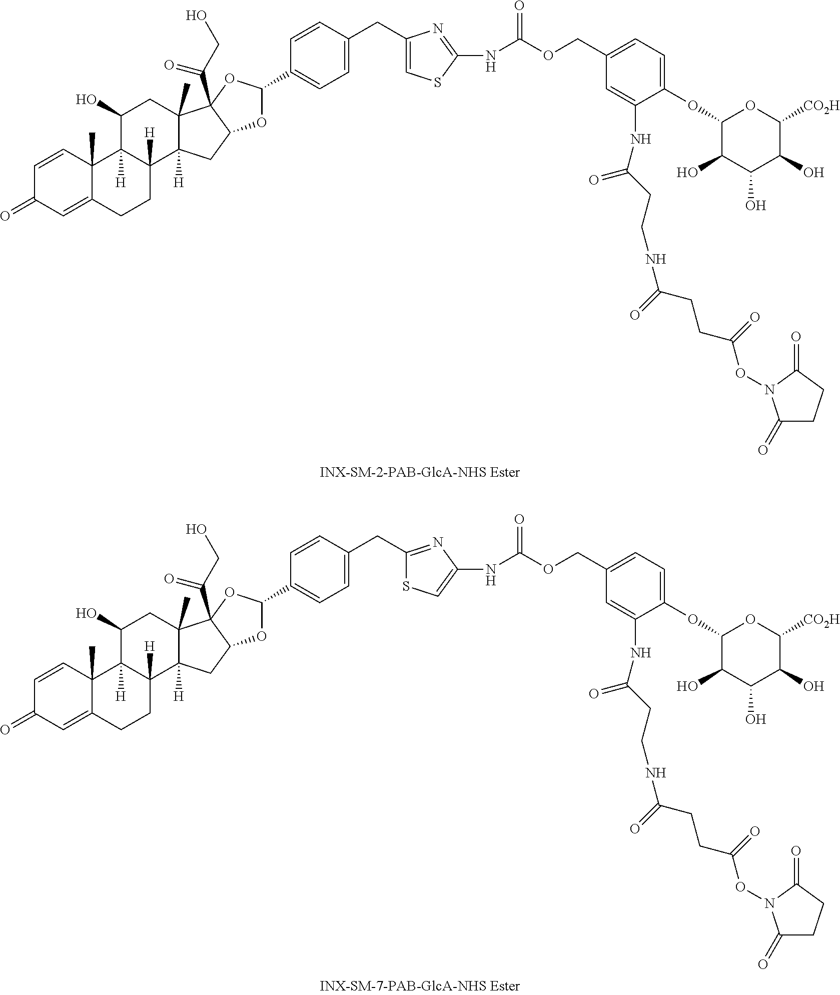

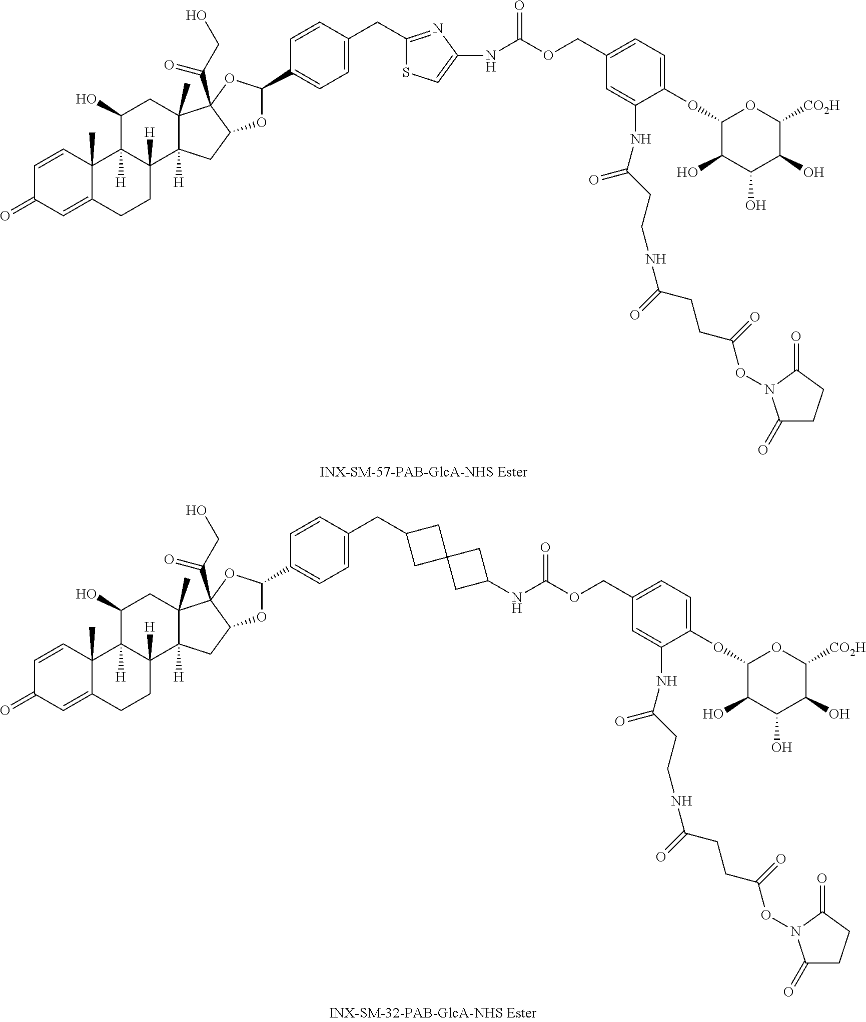

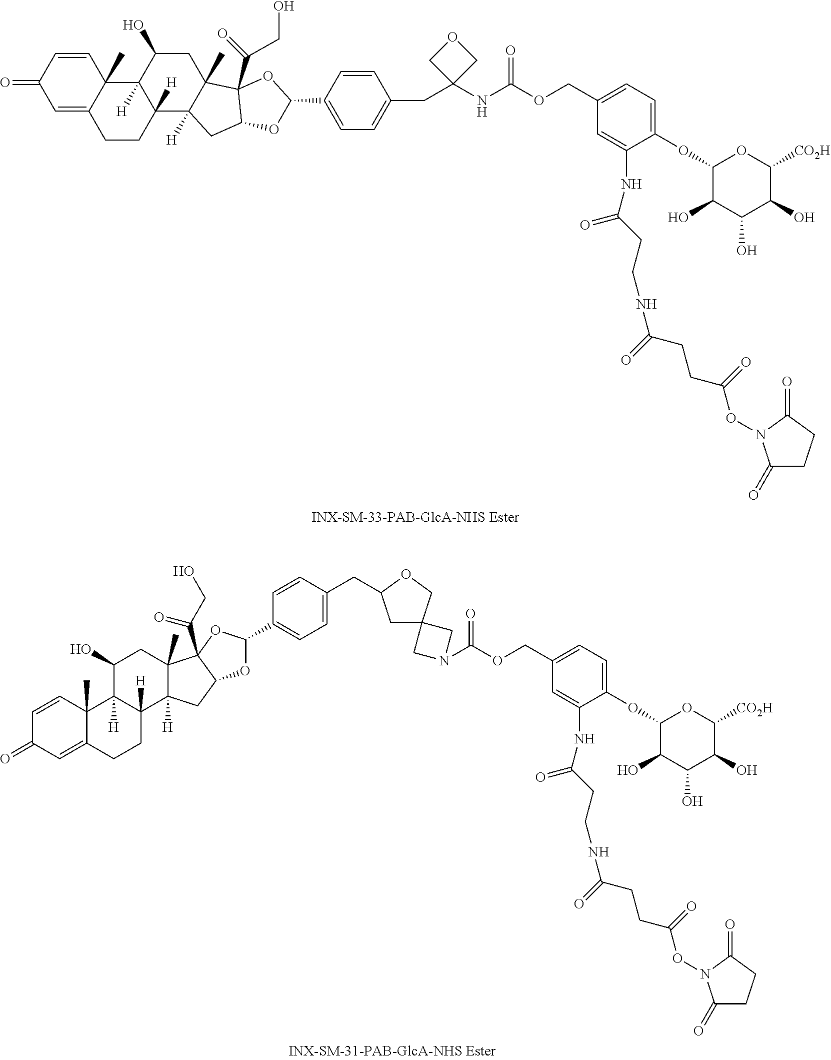

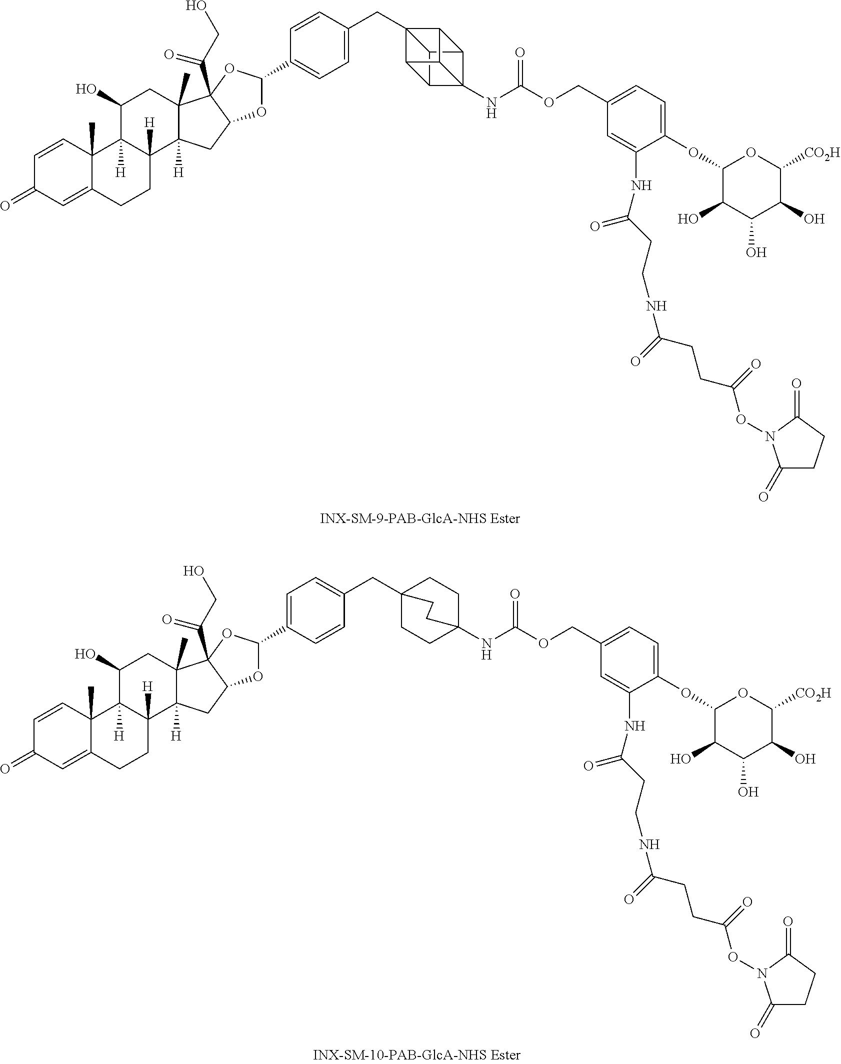

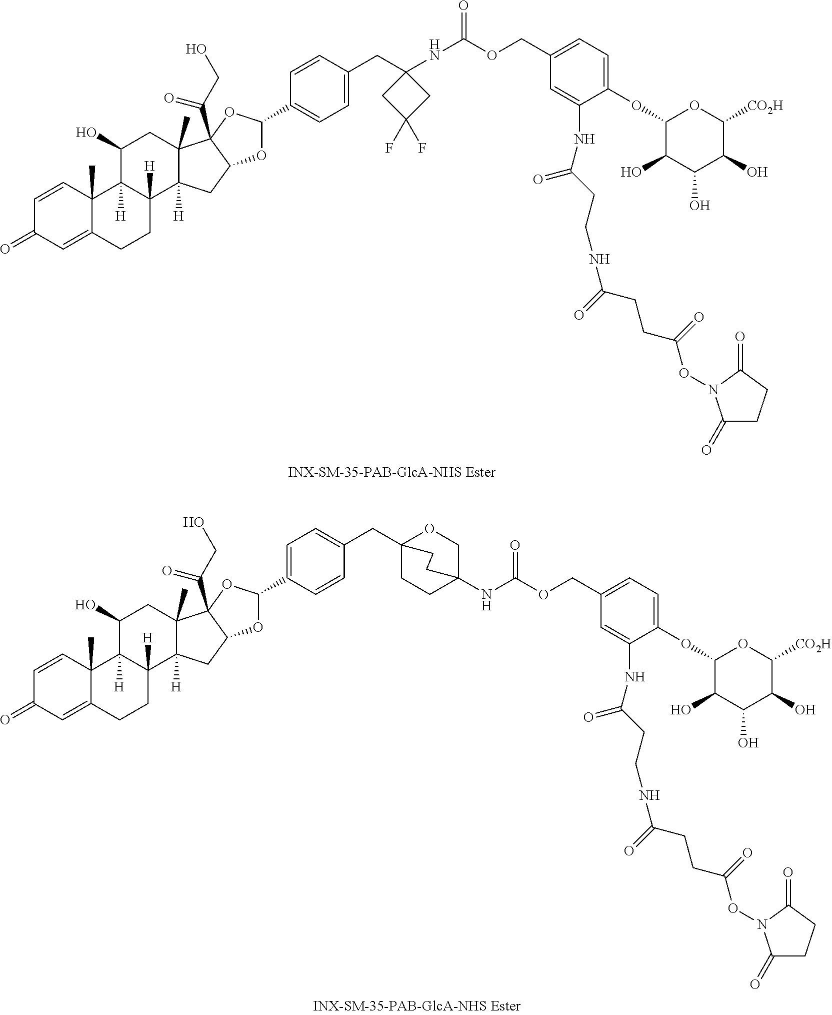

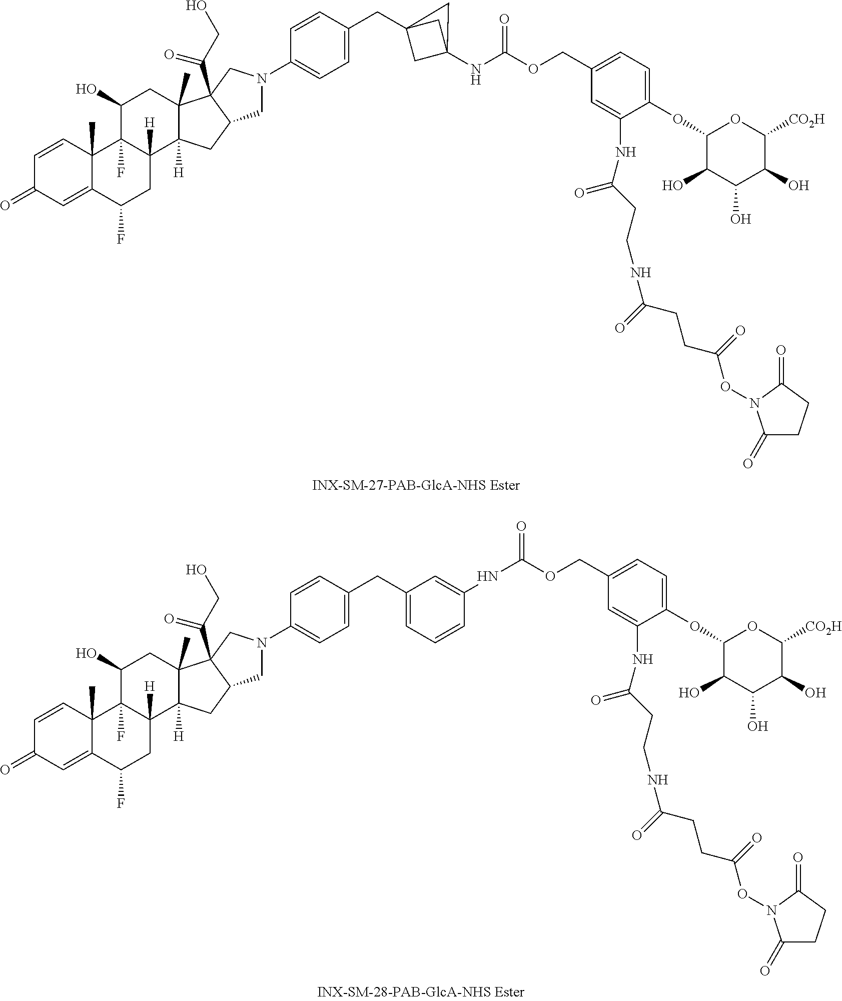

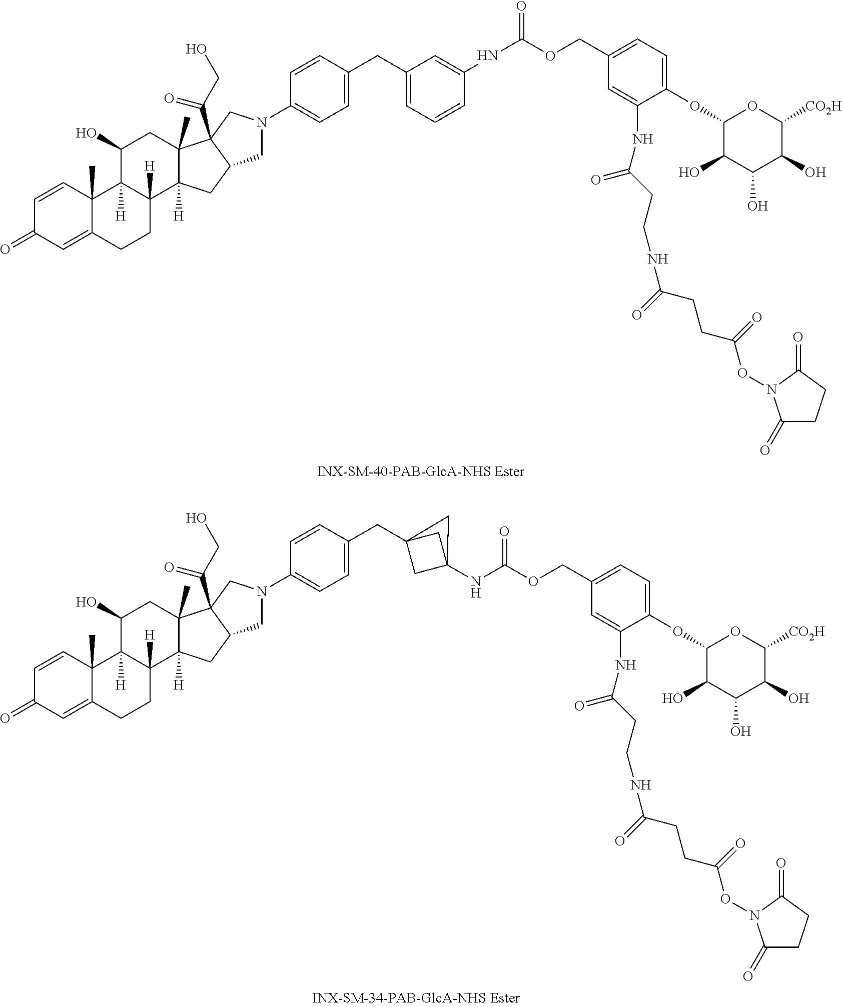

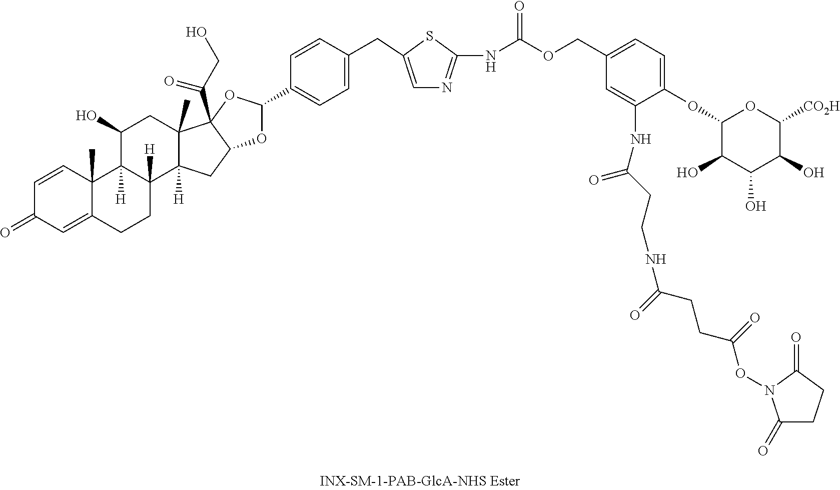

Wherein,
Linker=Glucuronidase cleavable sugar (GlcA)+Immolative linker Para Amino Benzyl (PAB)
J=Maleimide

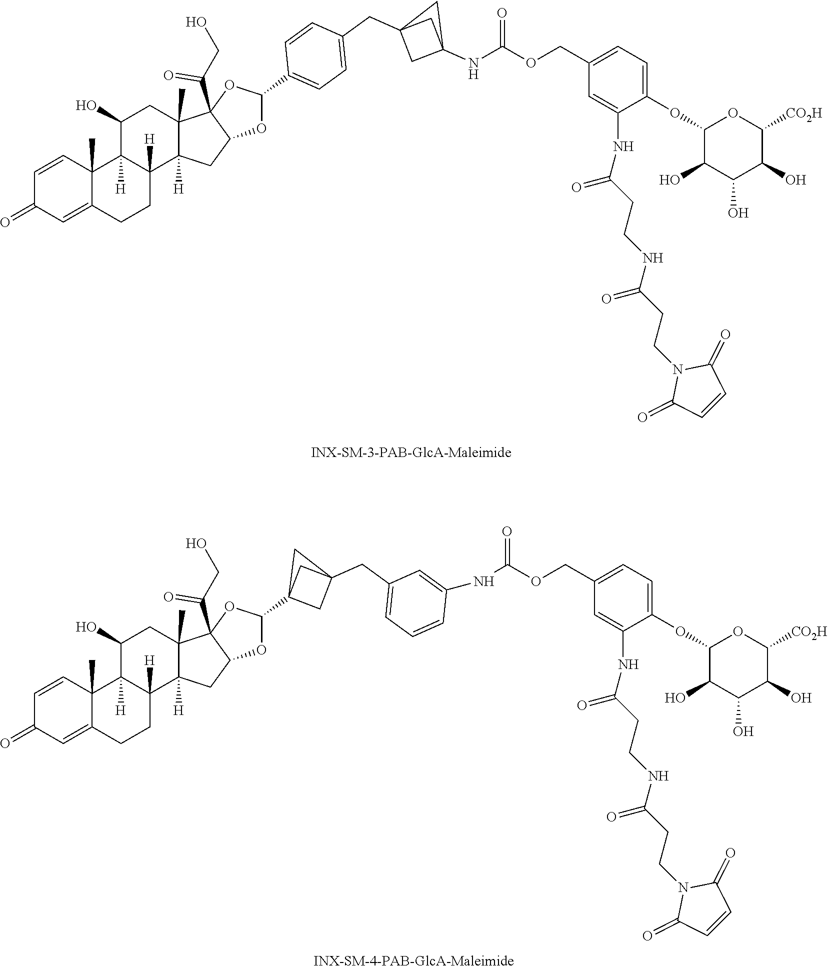

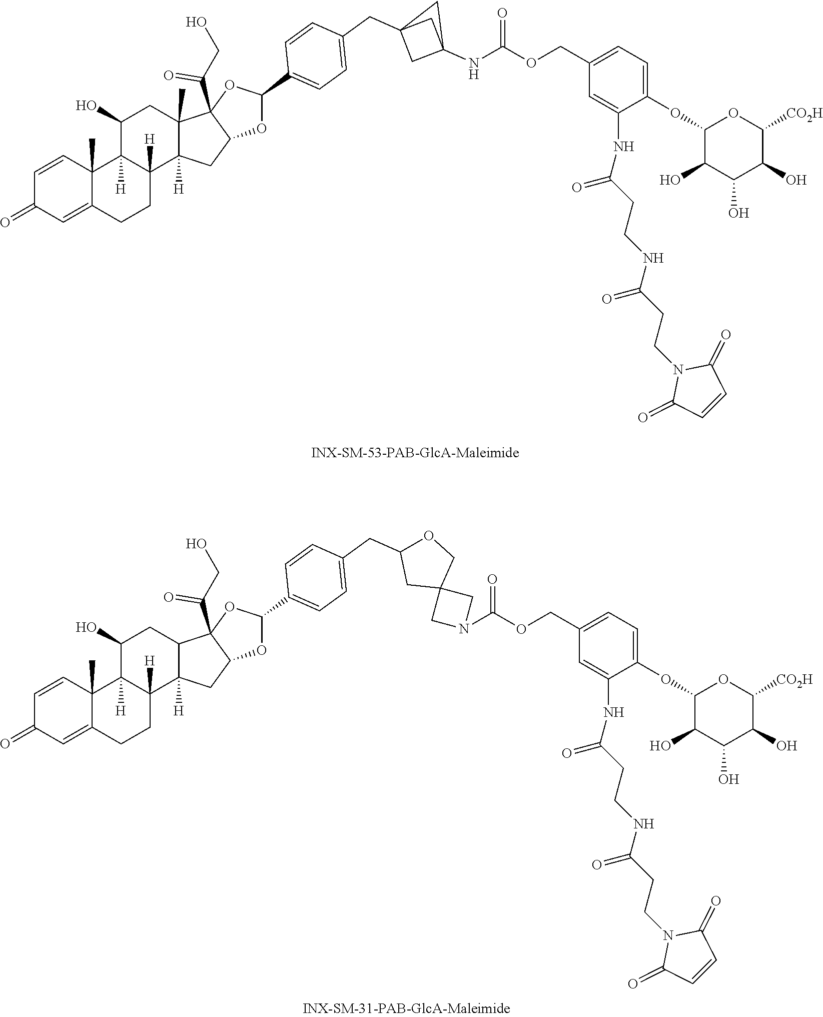

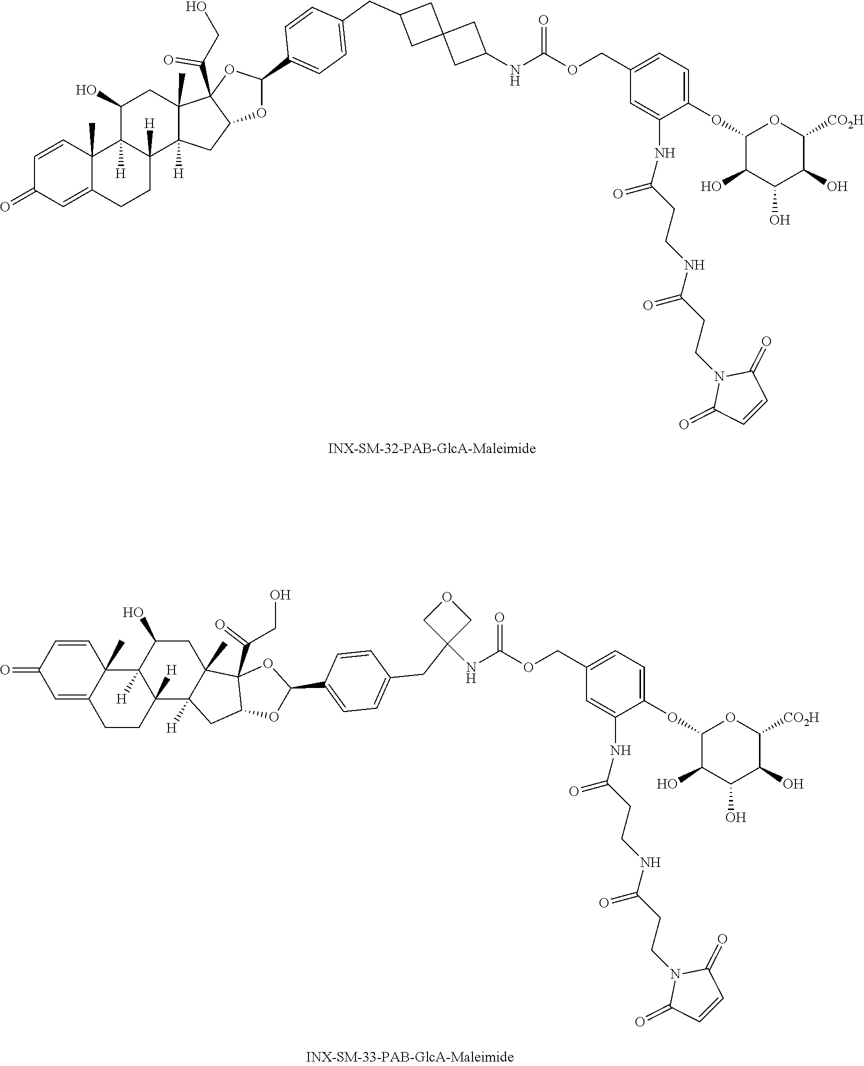

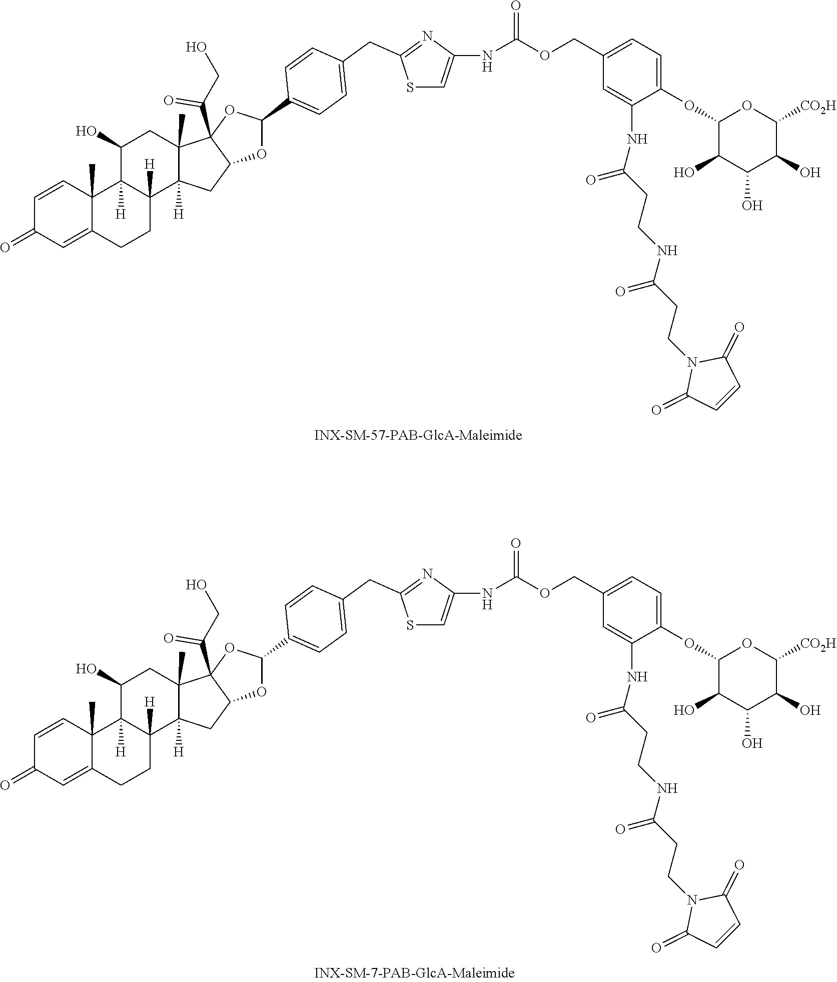

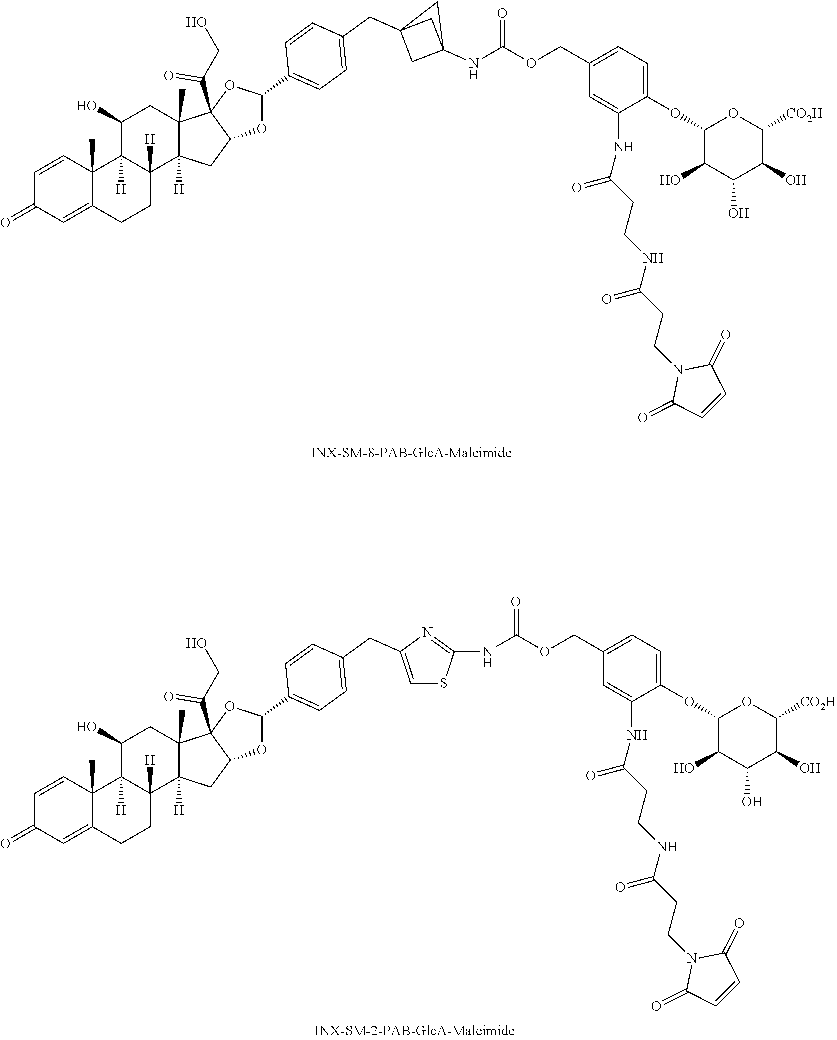

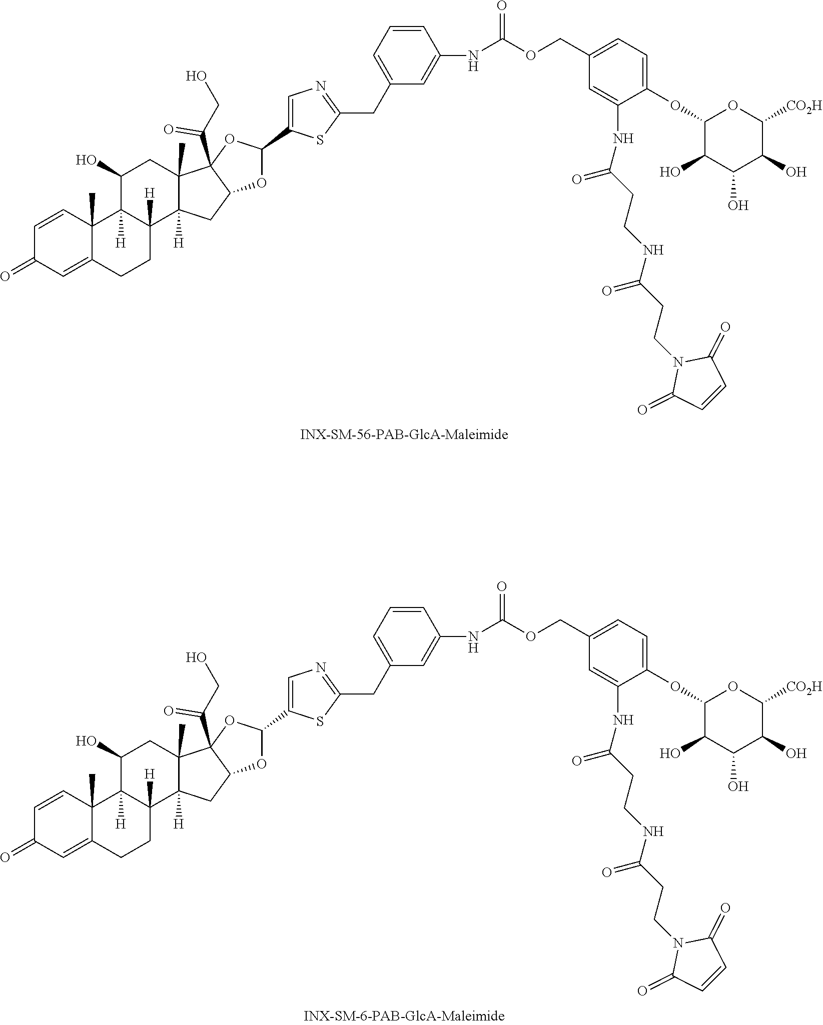

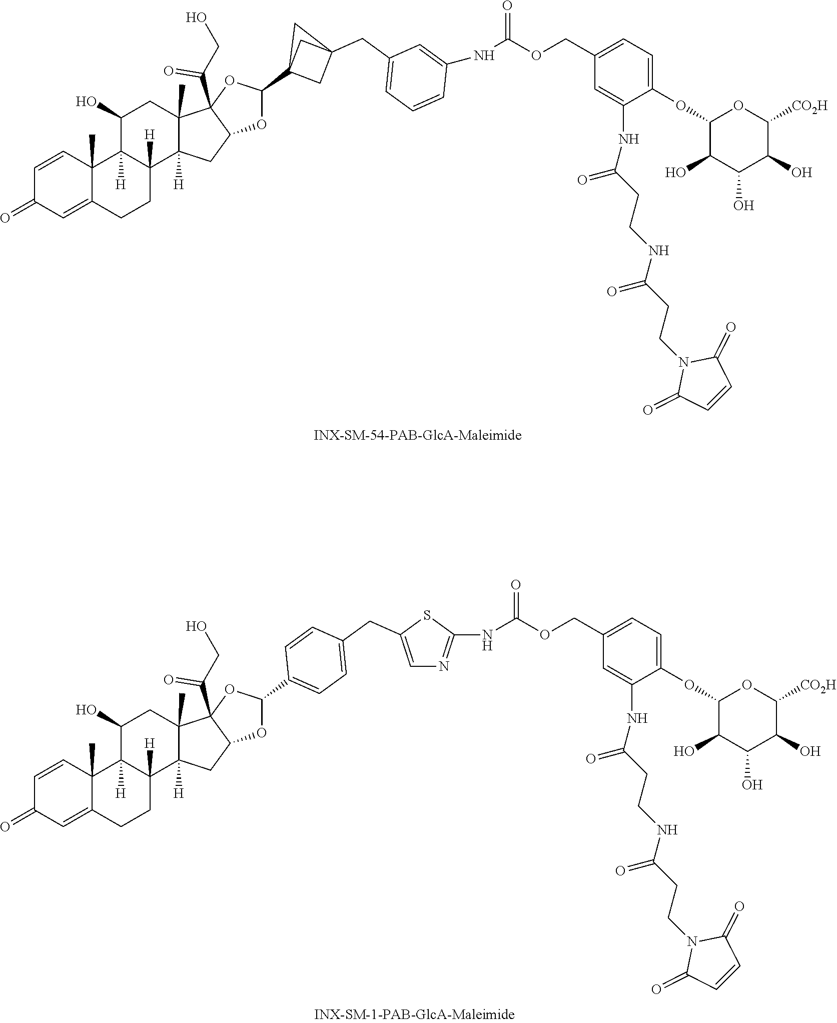

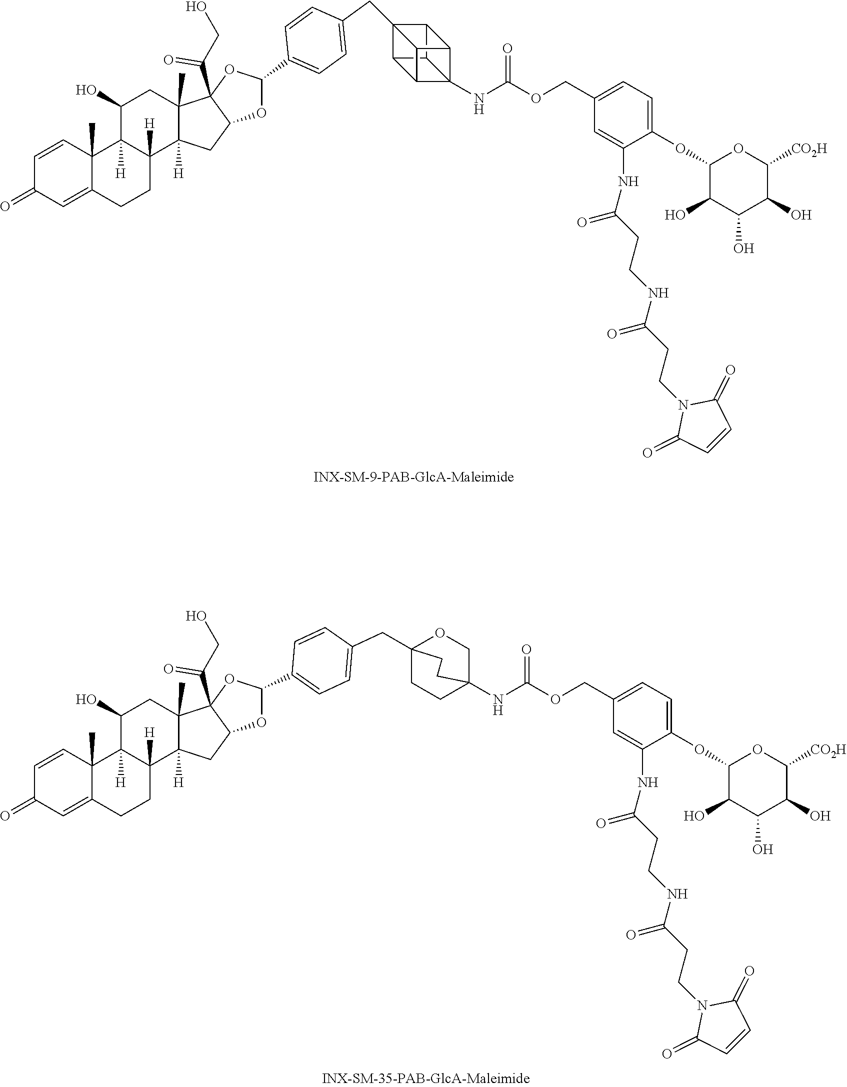

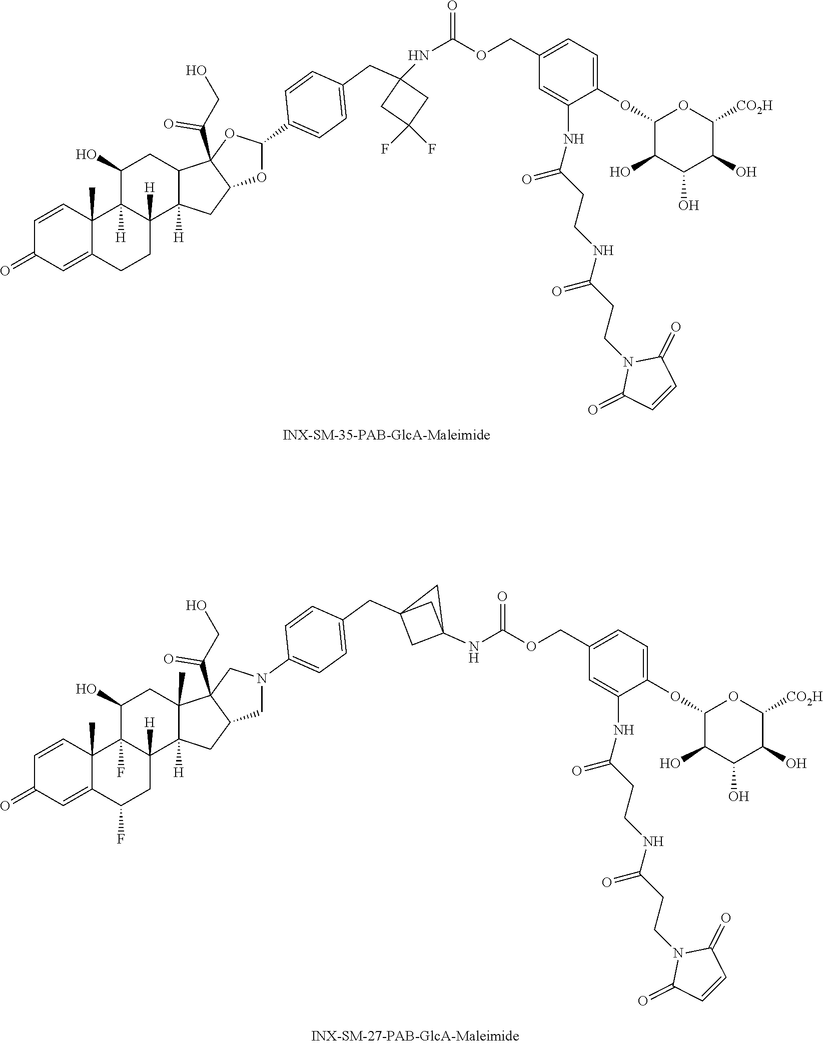

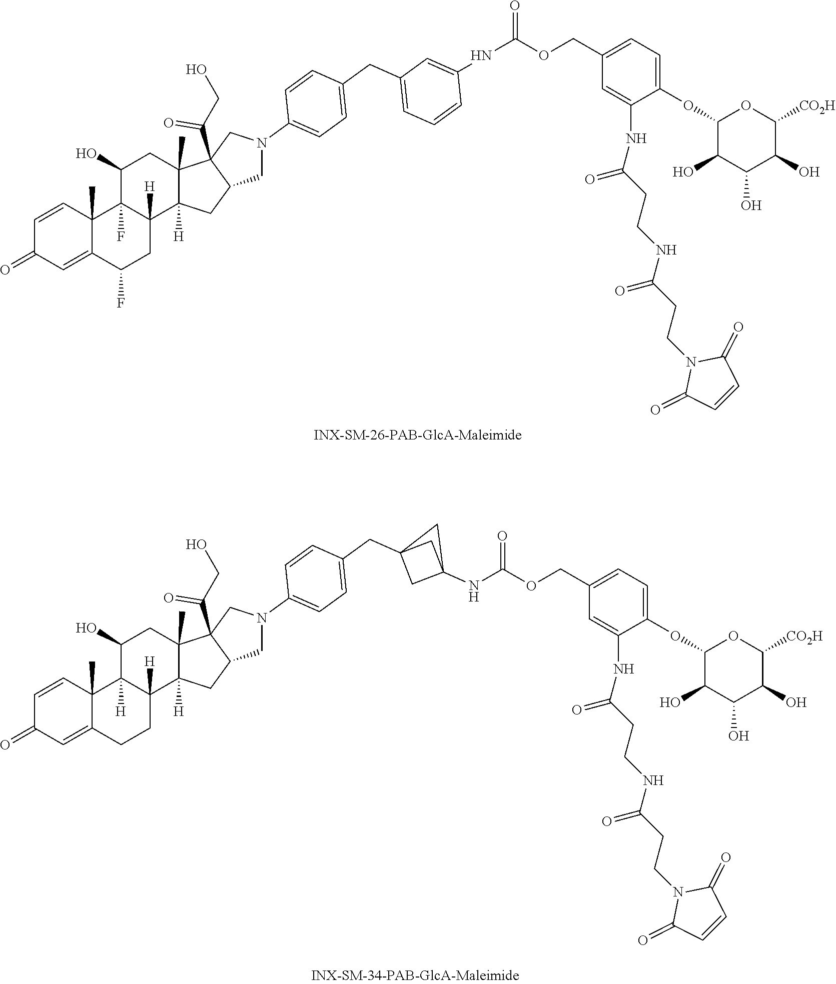

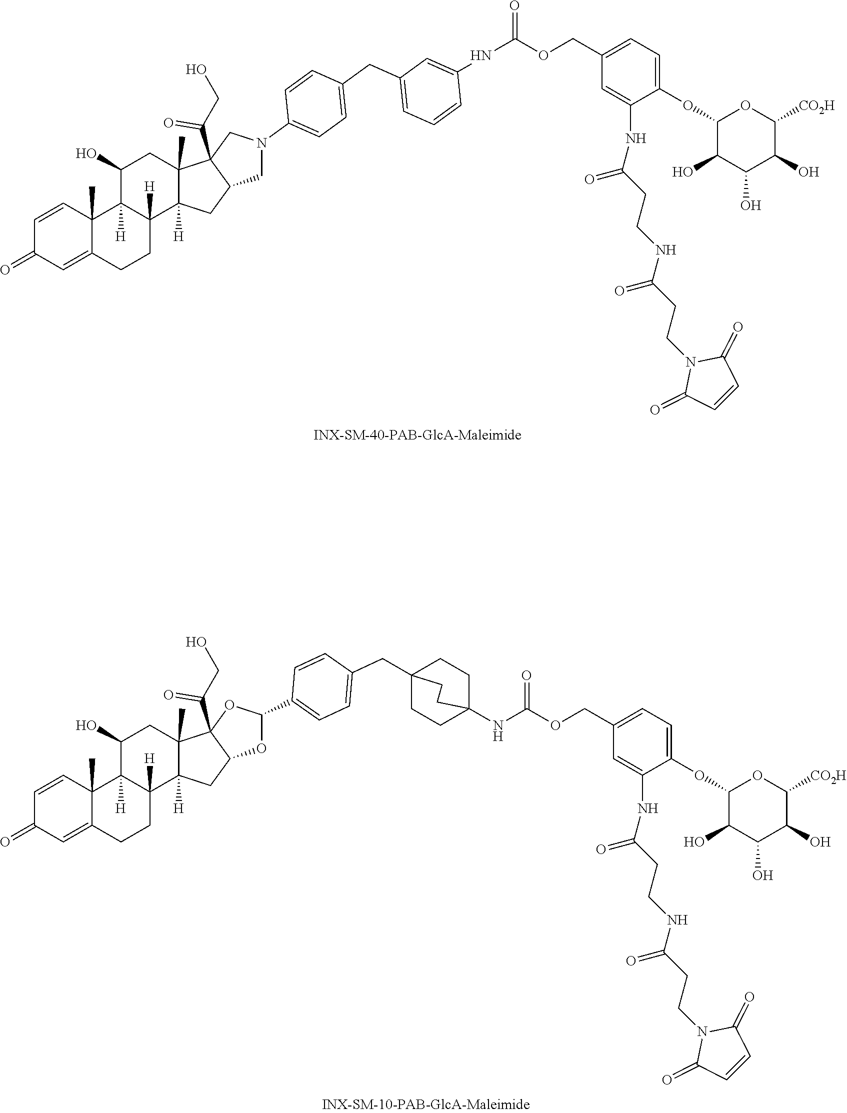

Where,
Linker=Glucuronidase cleavable sugar (GlcA)+Immolative linker Para Amino Benzyl (PAB)
J=Tetrazine

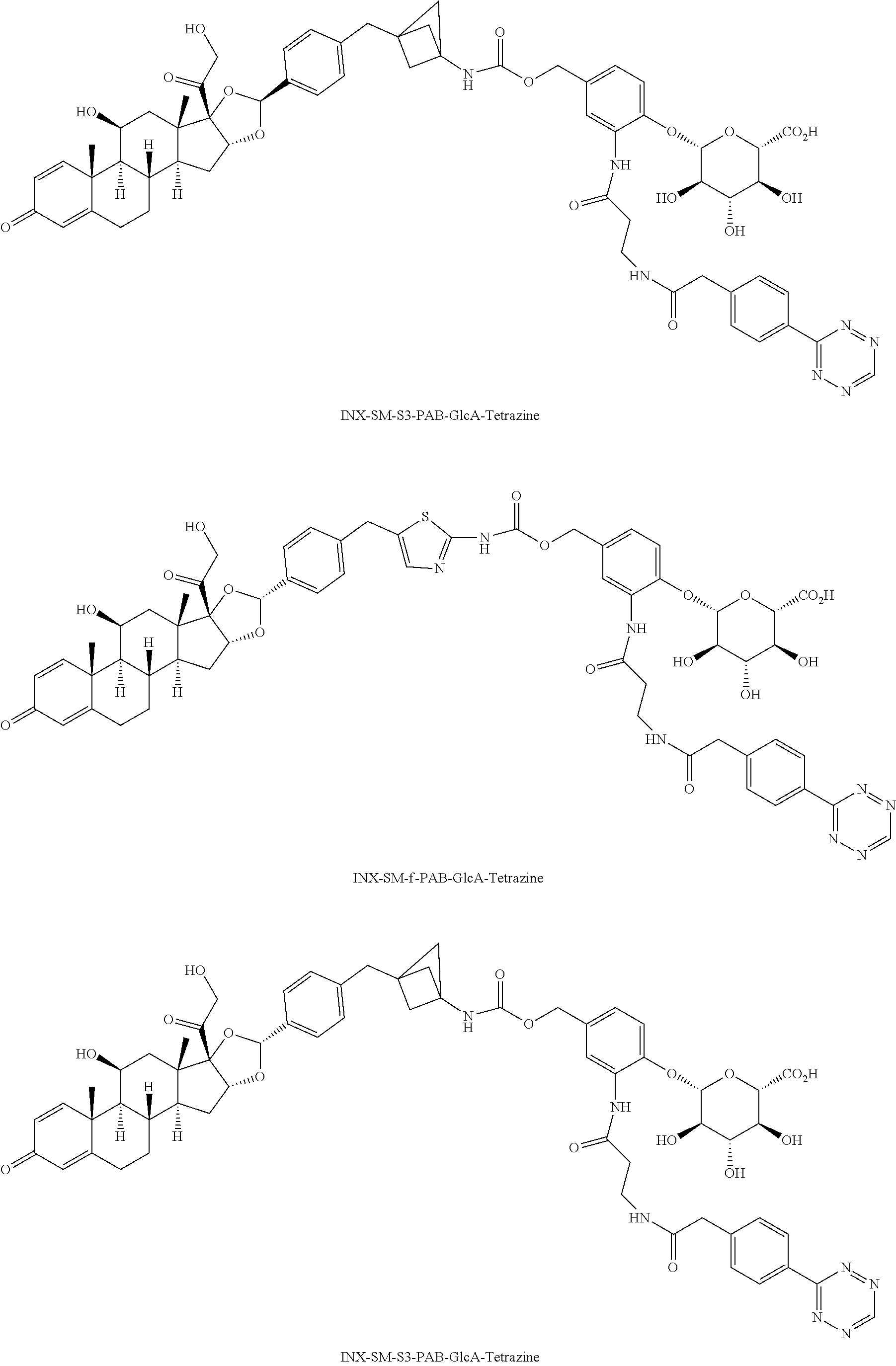

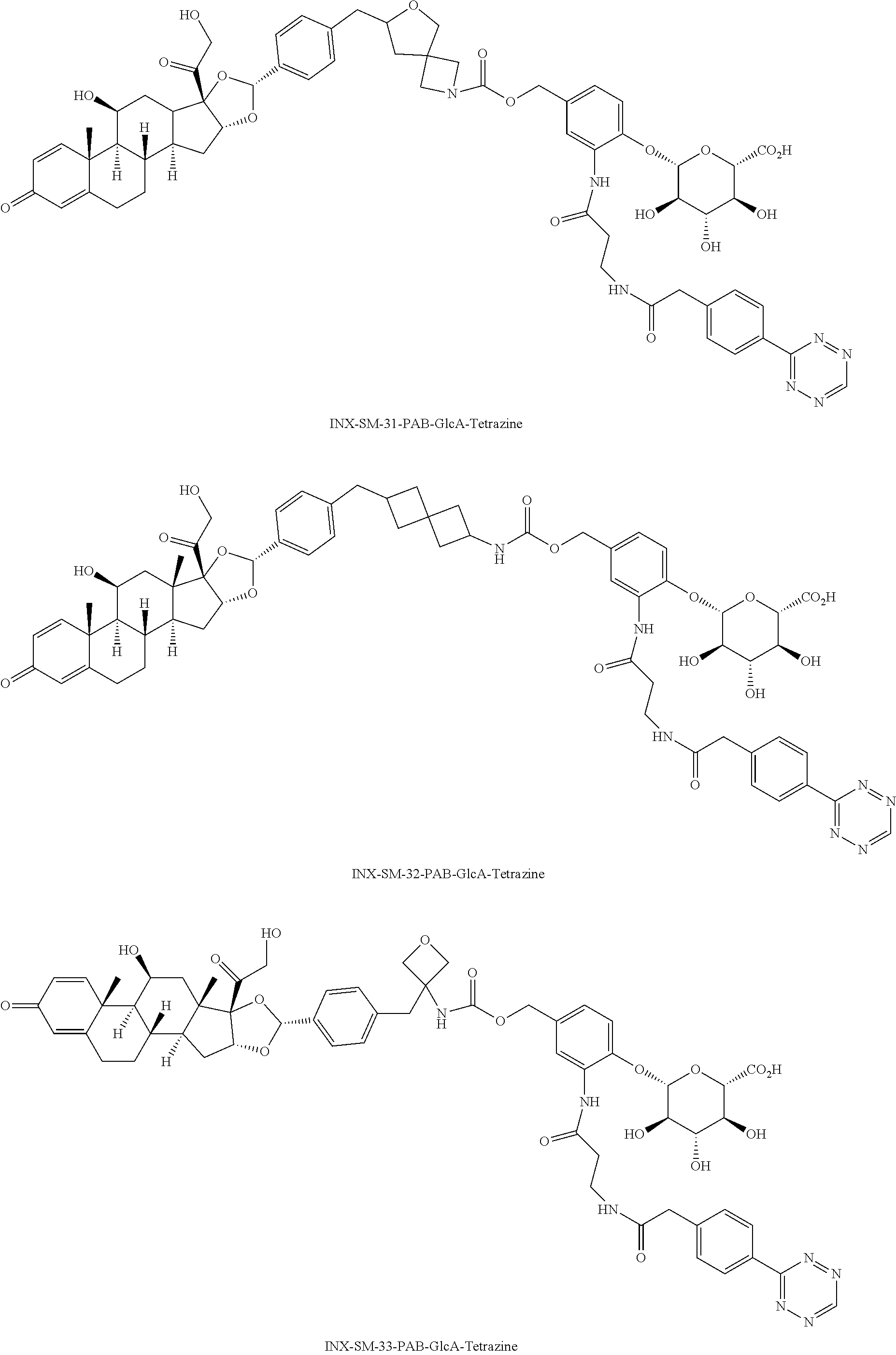

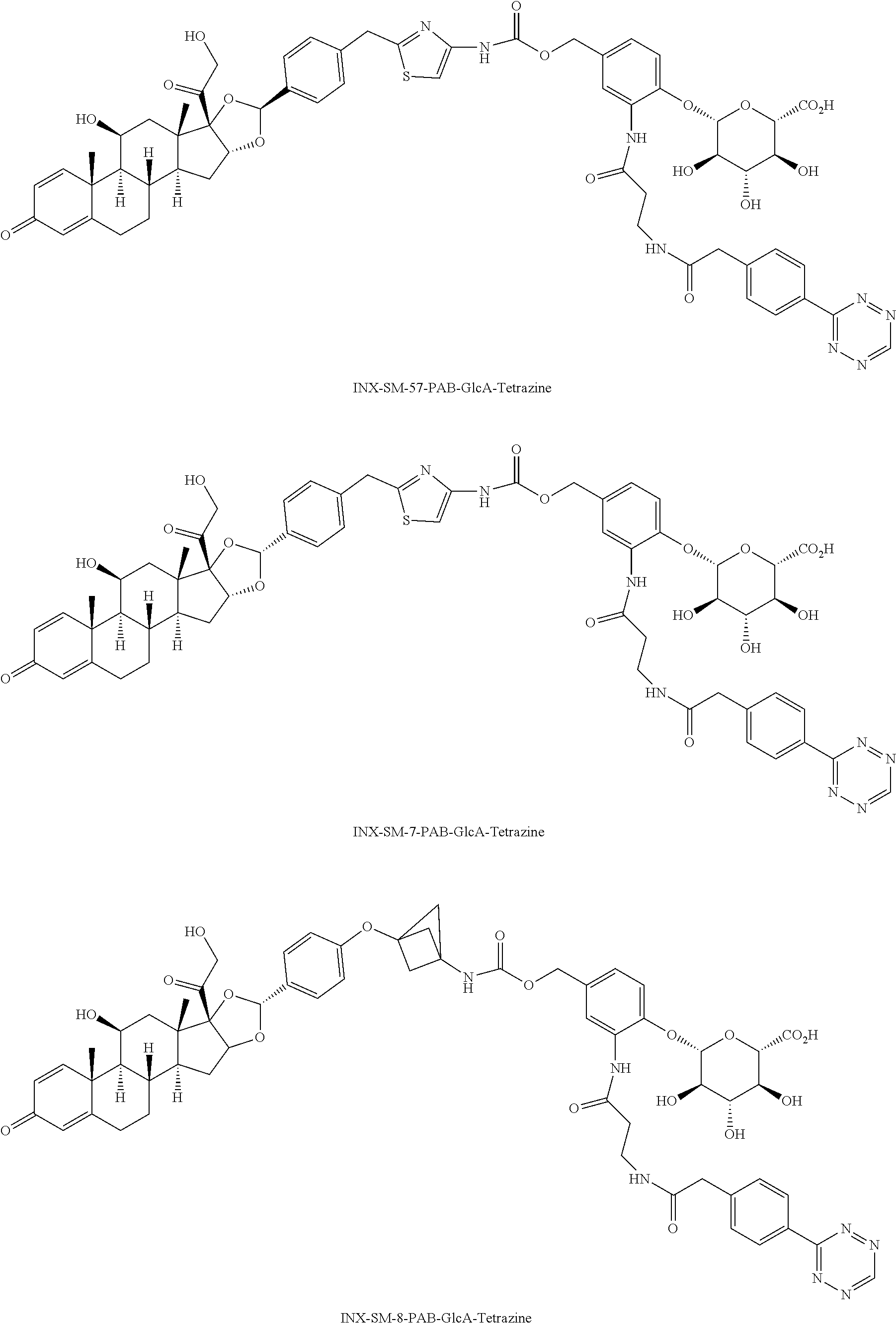

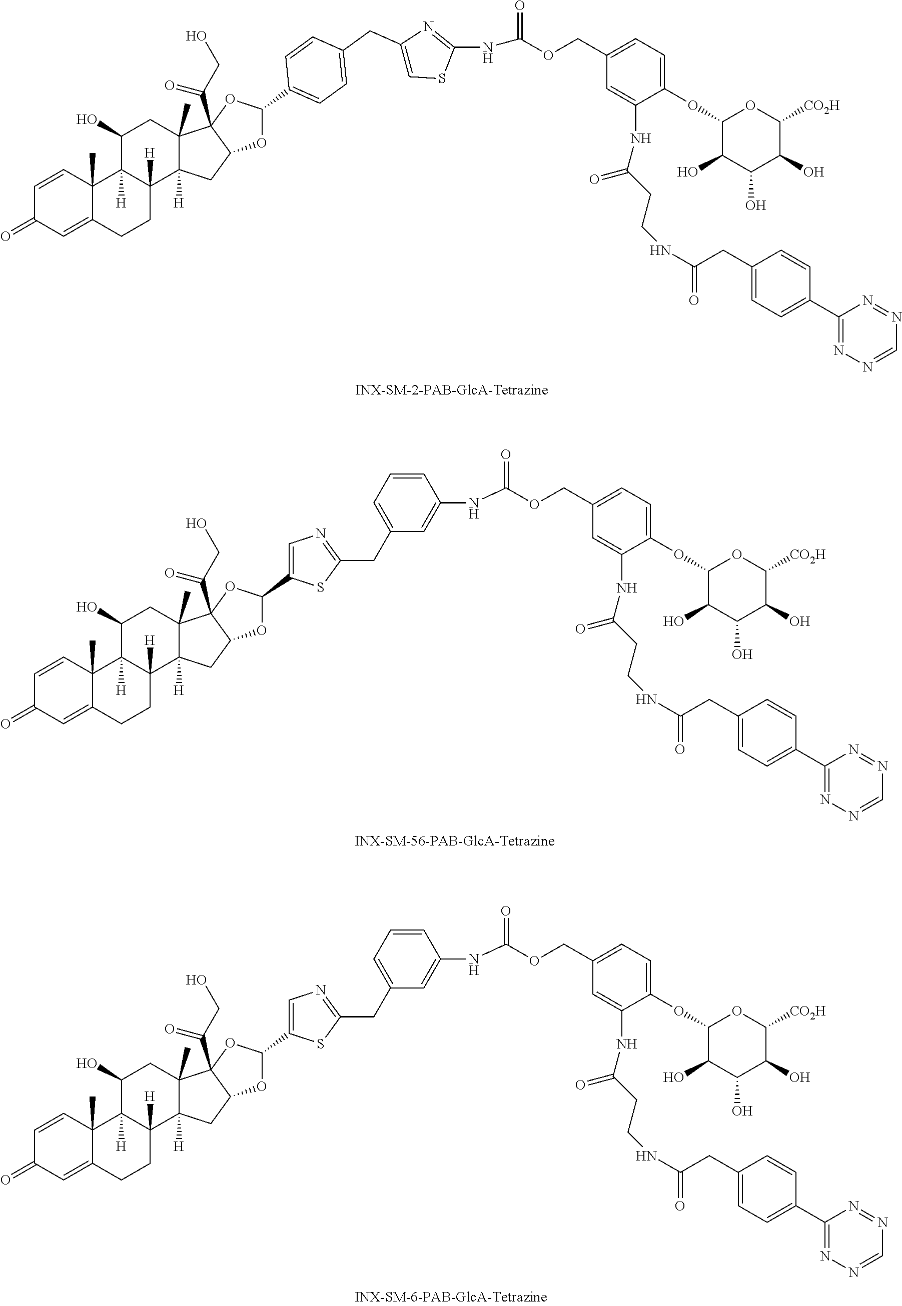

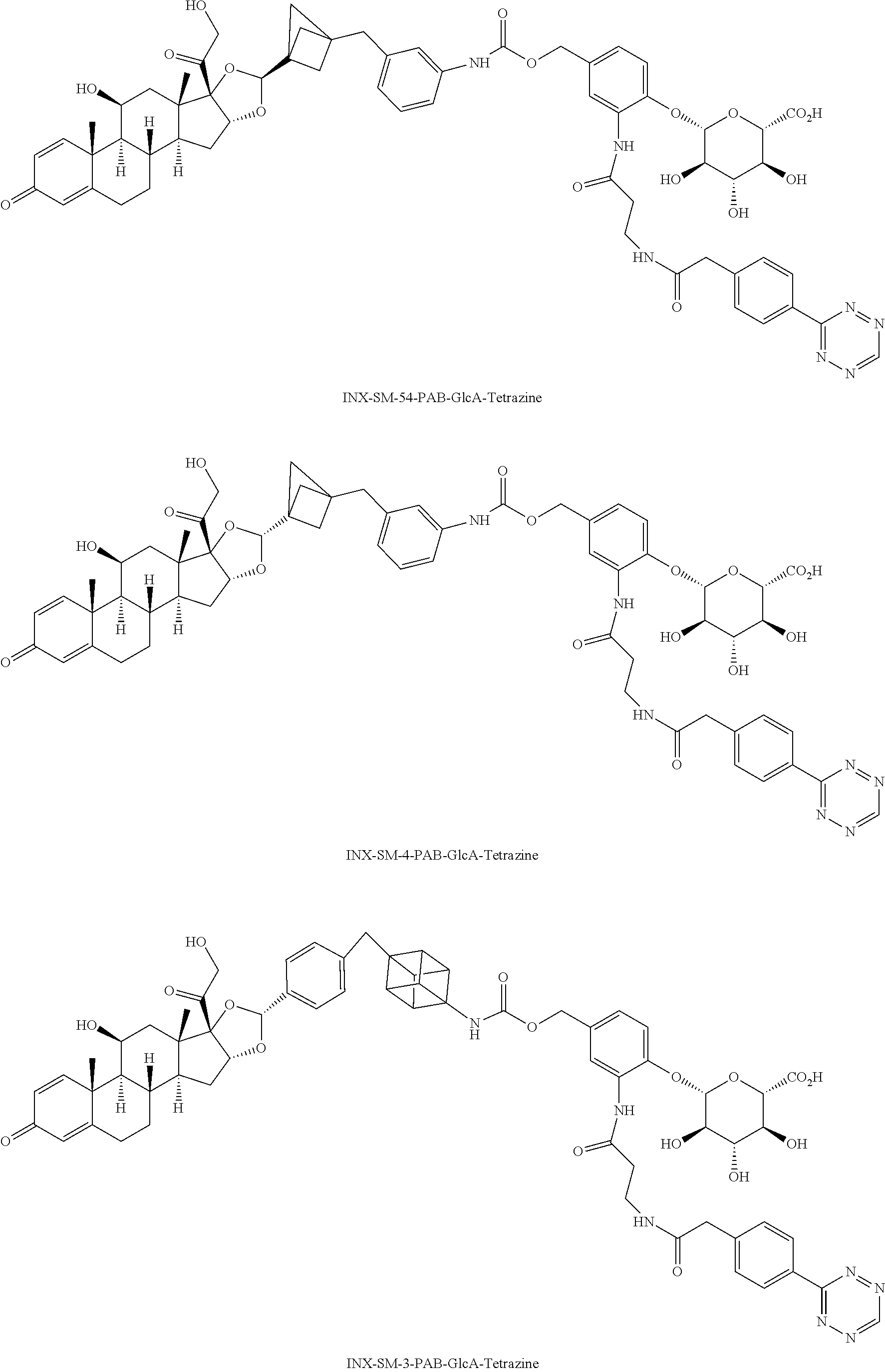

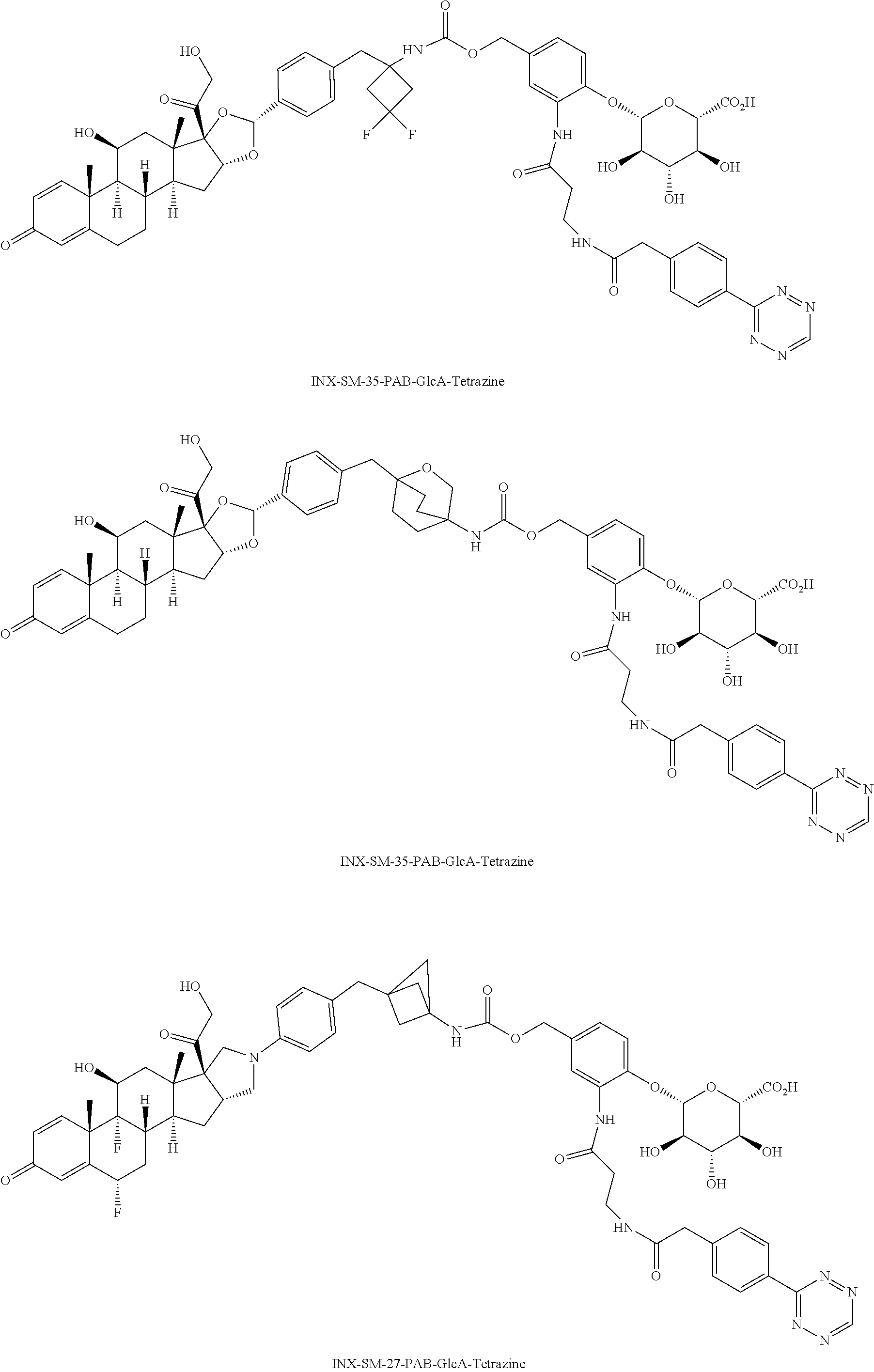

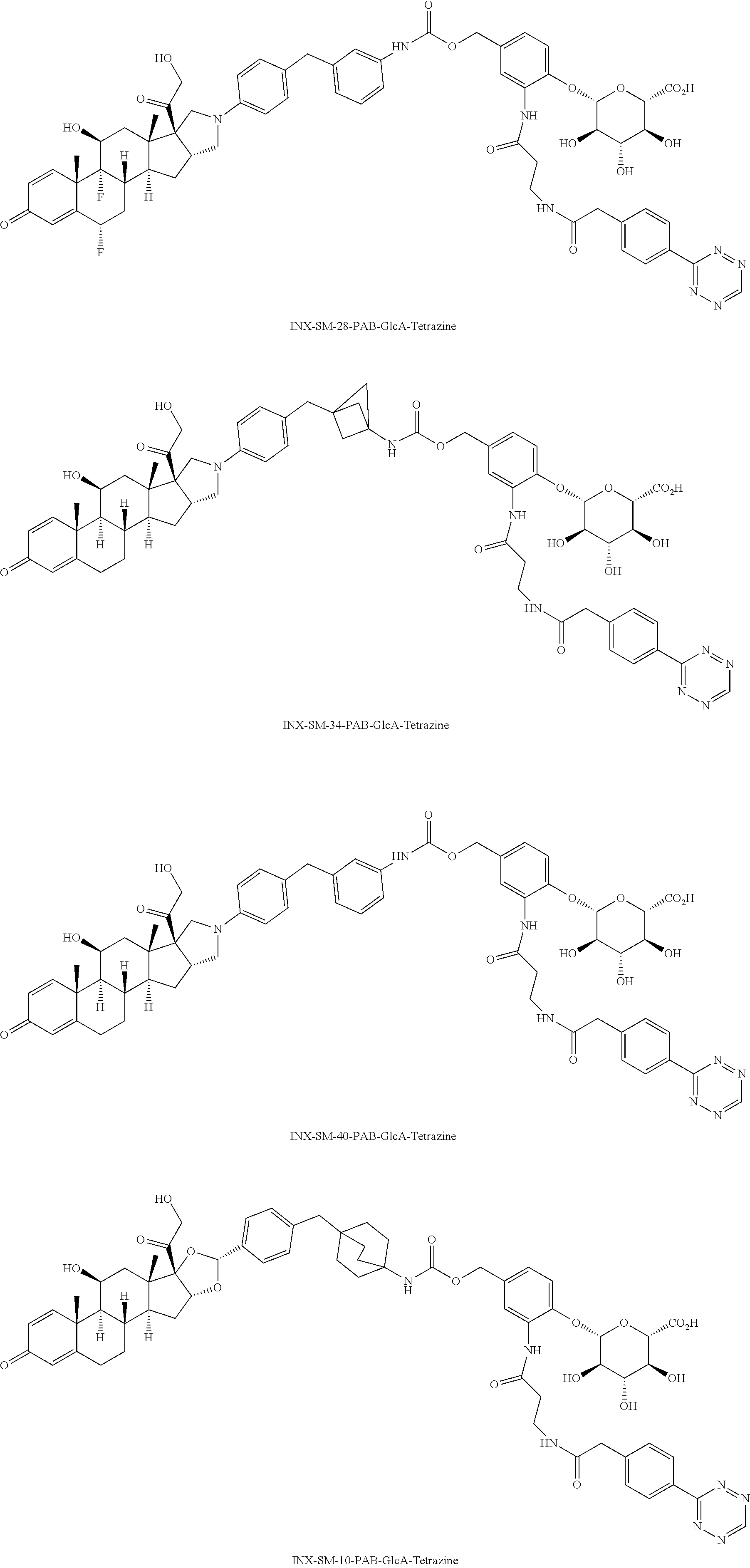

Where,

Linker=Glucuronidase cleavable sugar (GlcA)+Immolative linker Para Amino Benzyl (PAB)
J=Amine

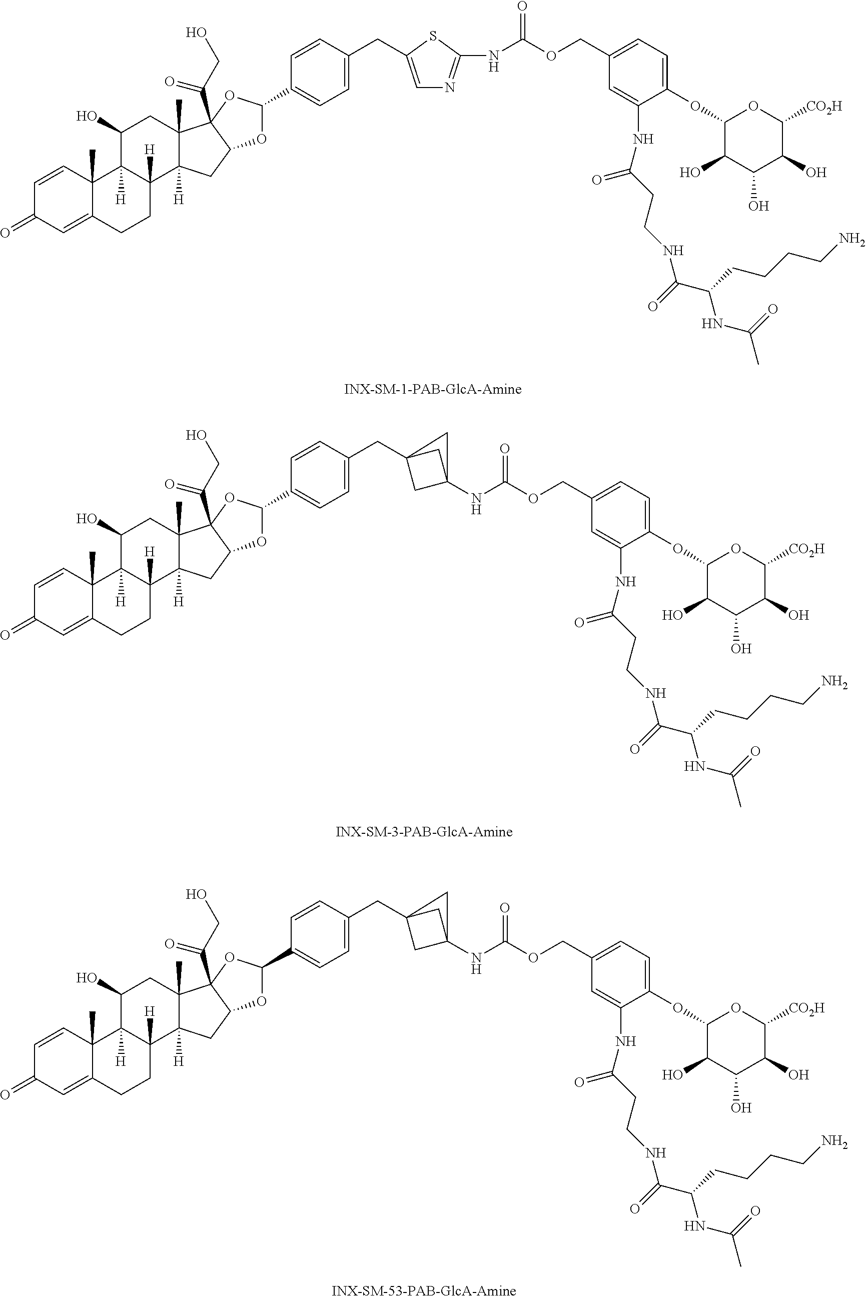

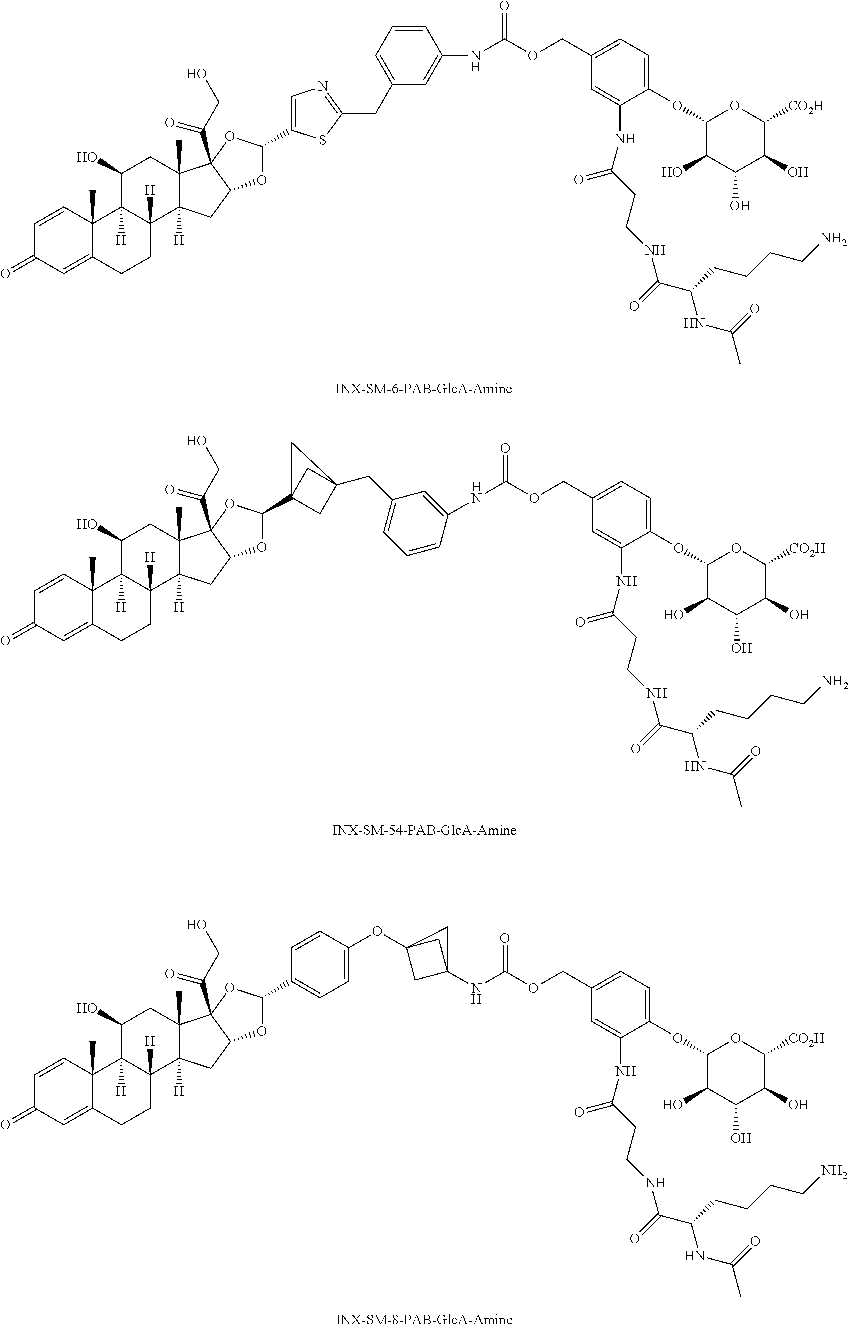

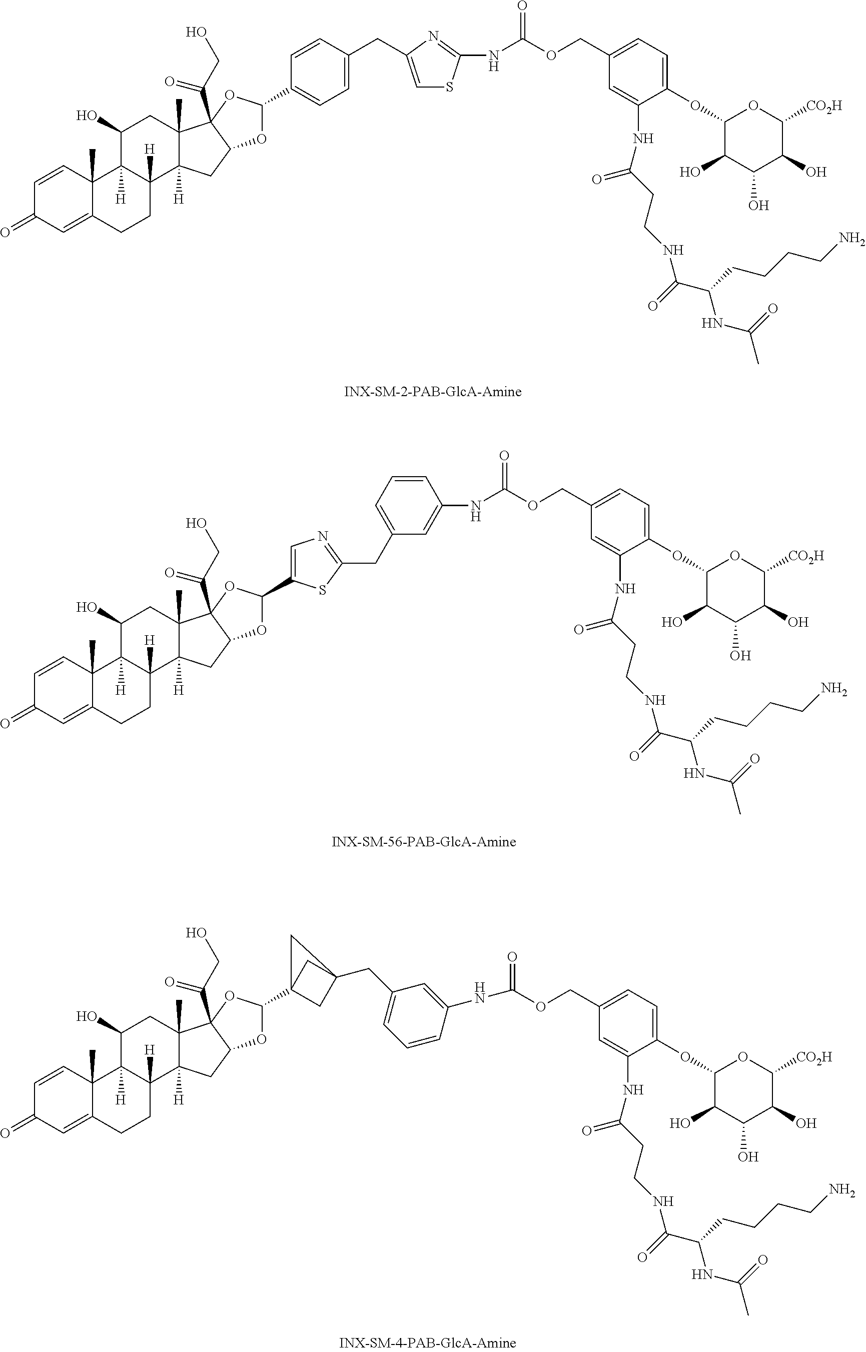

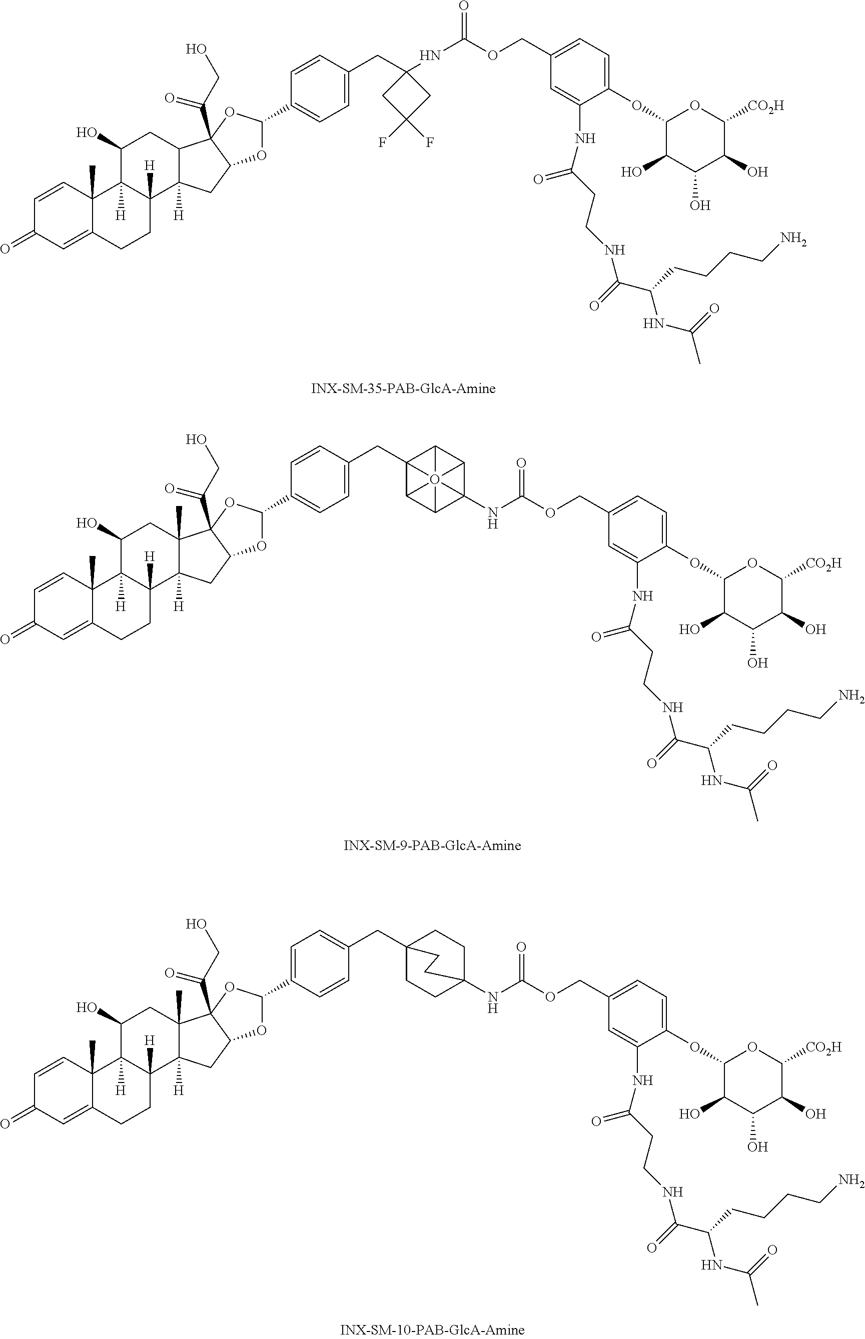

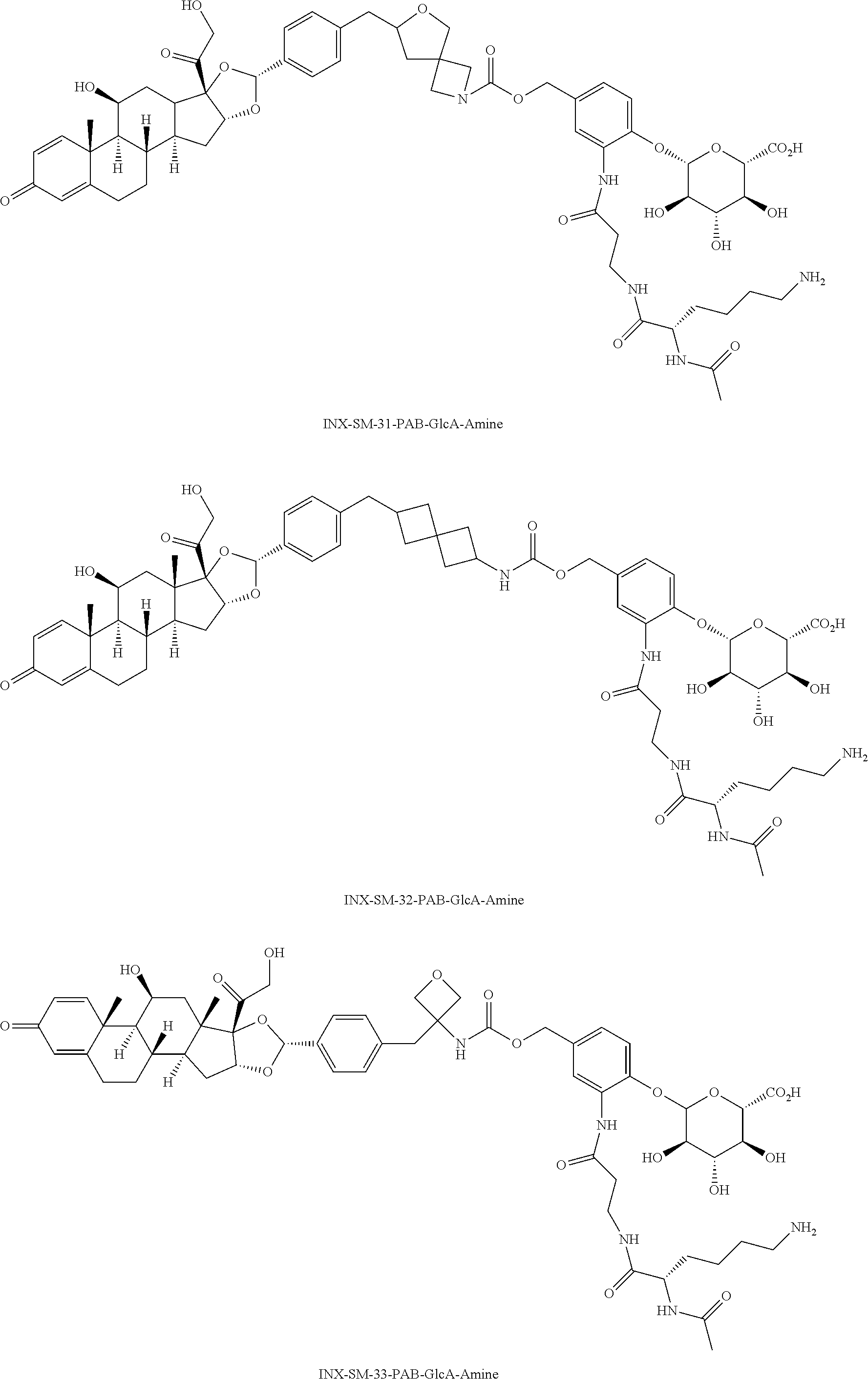

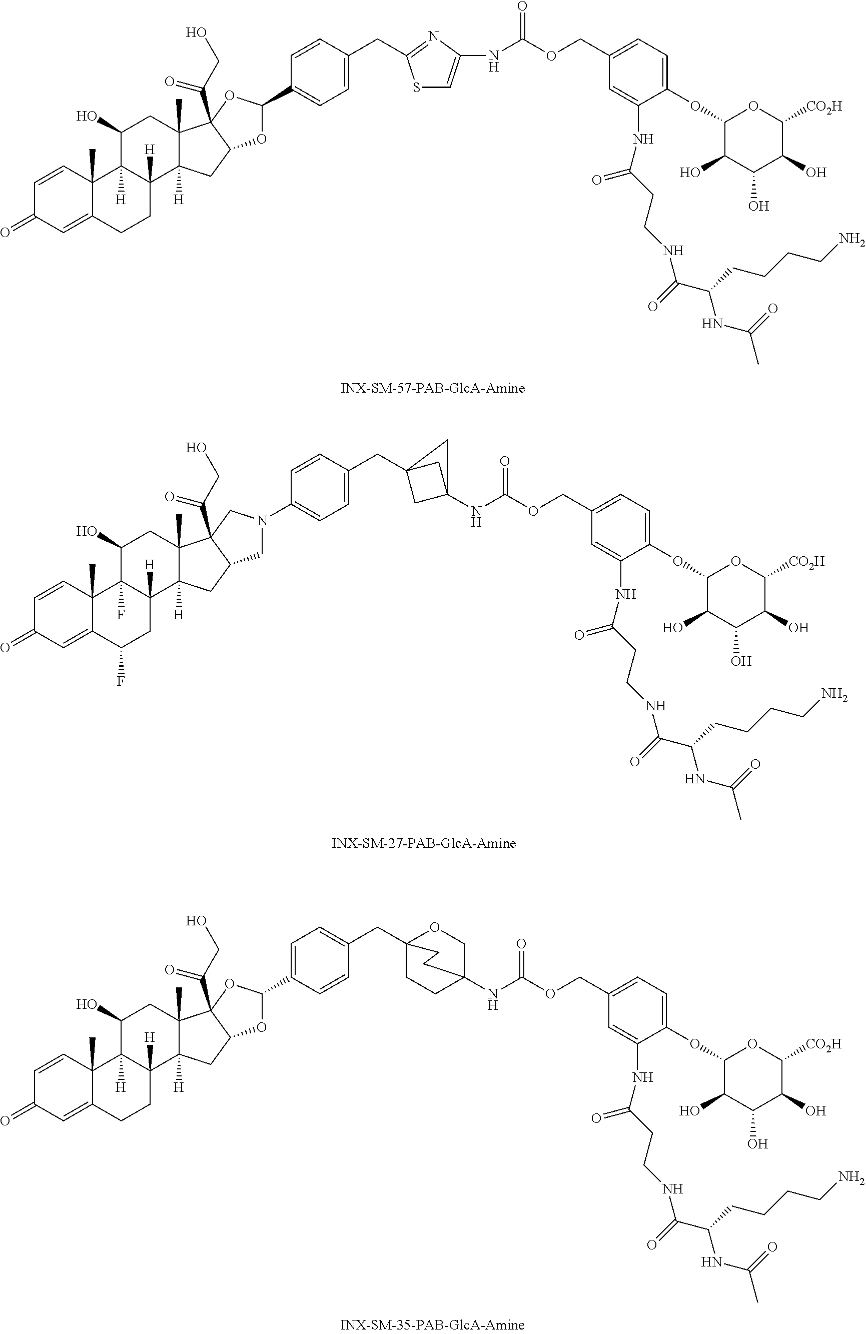

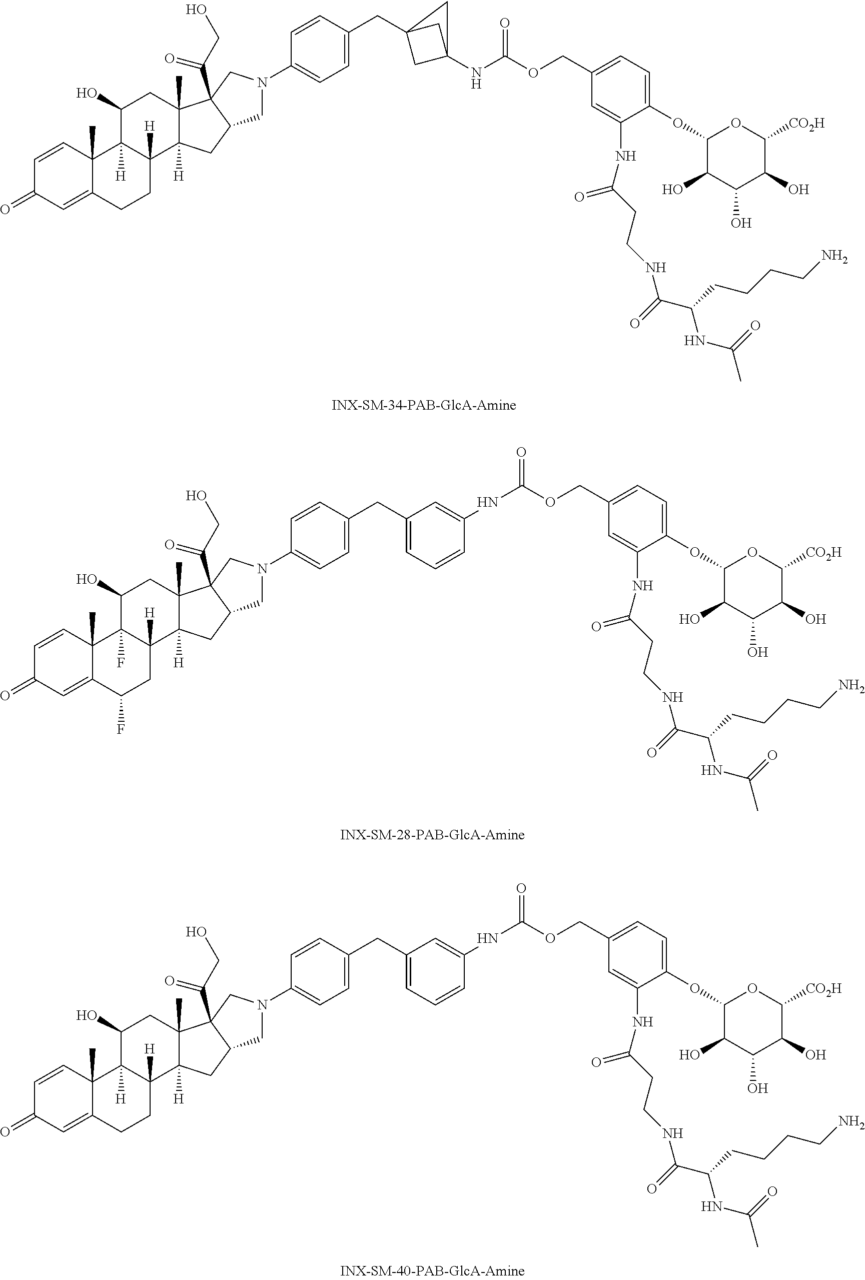

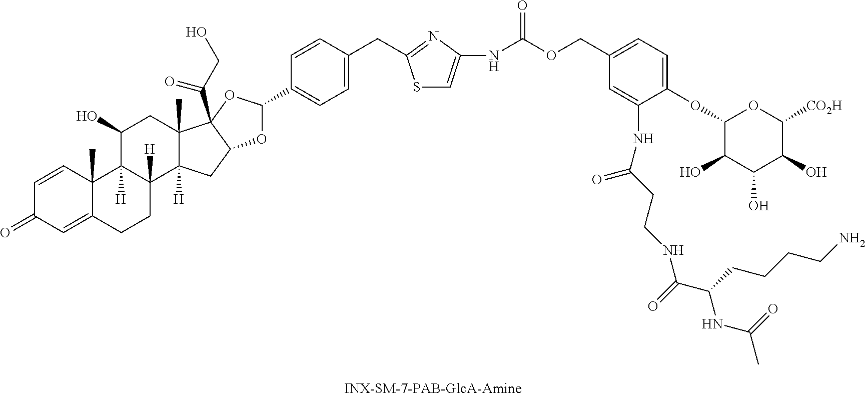

### (IV) Payload-Linker-J

### Alternative Site of Linker-J Attachment to Payload (C11-OH).

### INX-SM-3 is Used as a Payload Example

### Alkoxyamine

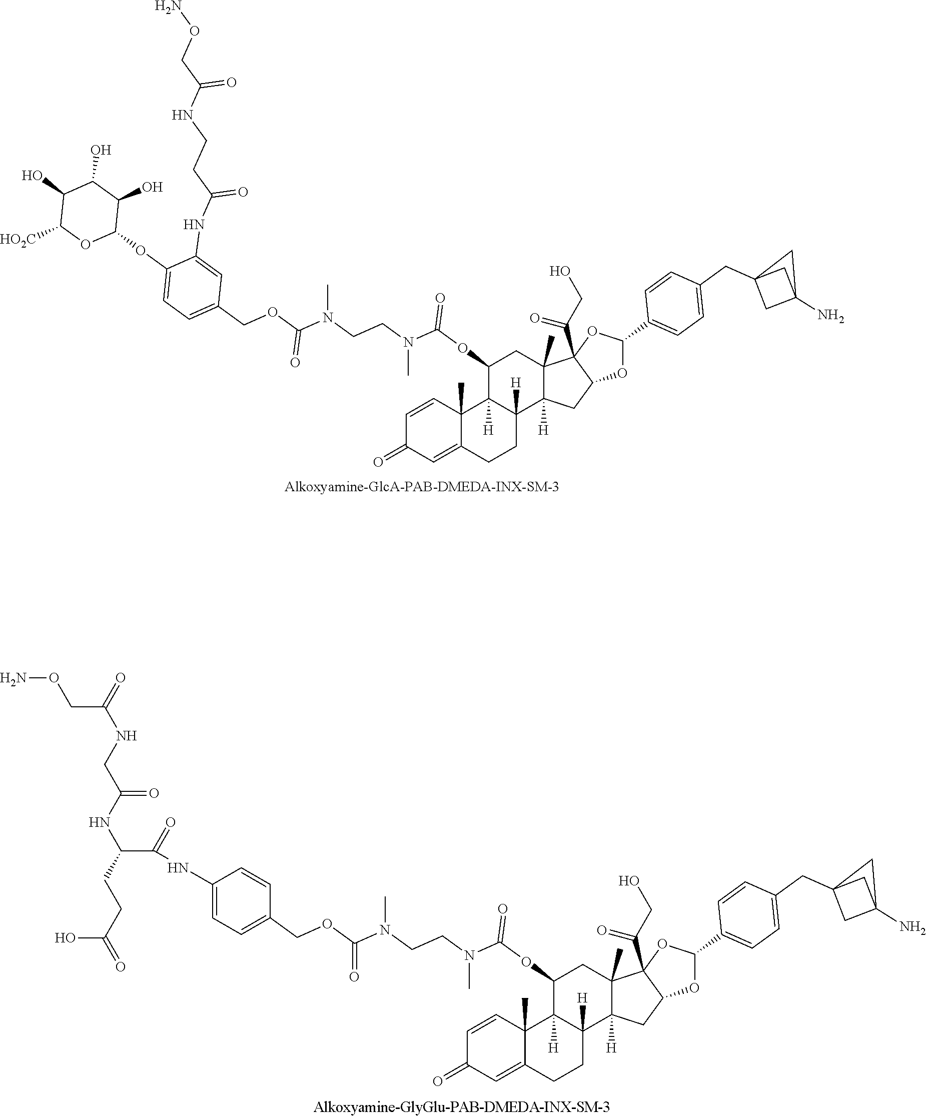

### Bromoacetyl

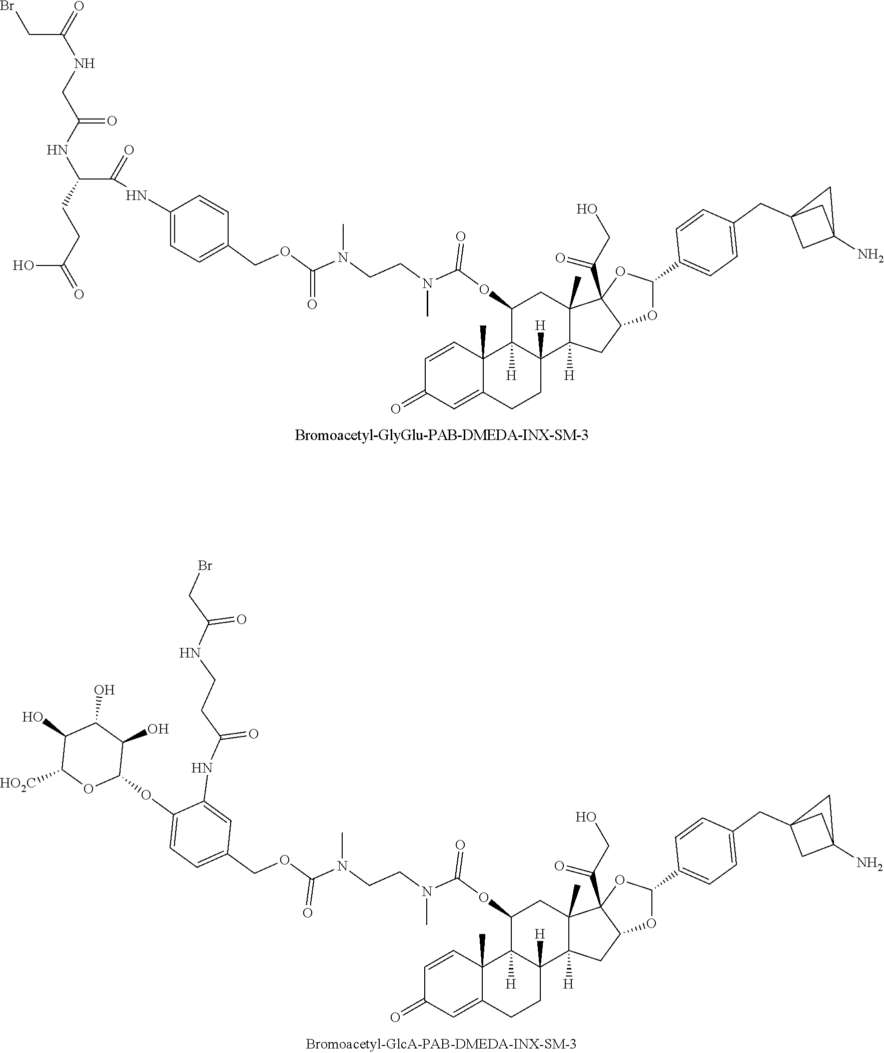

### Maleimide

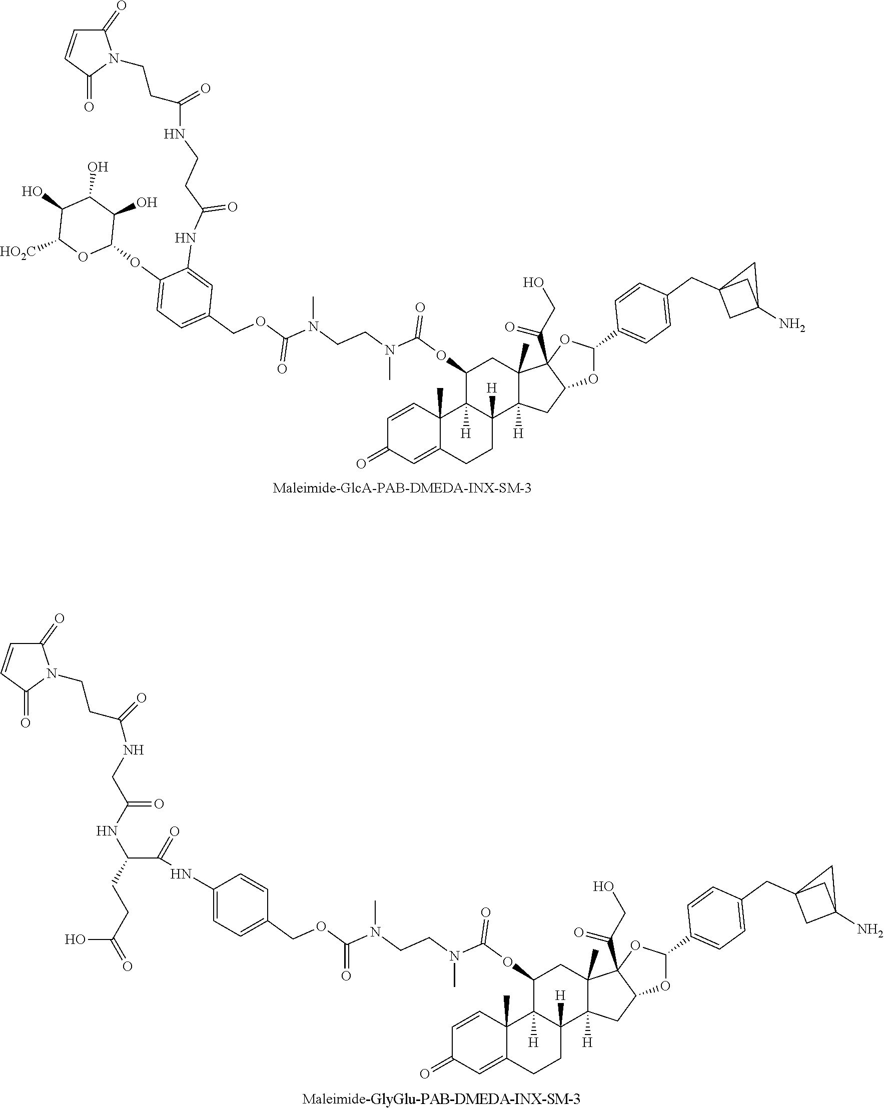

### Dibenzocyclooctyne

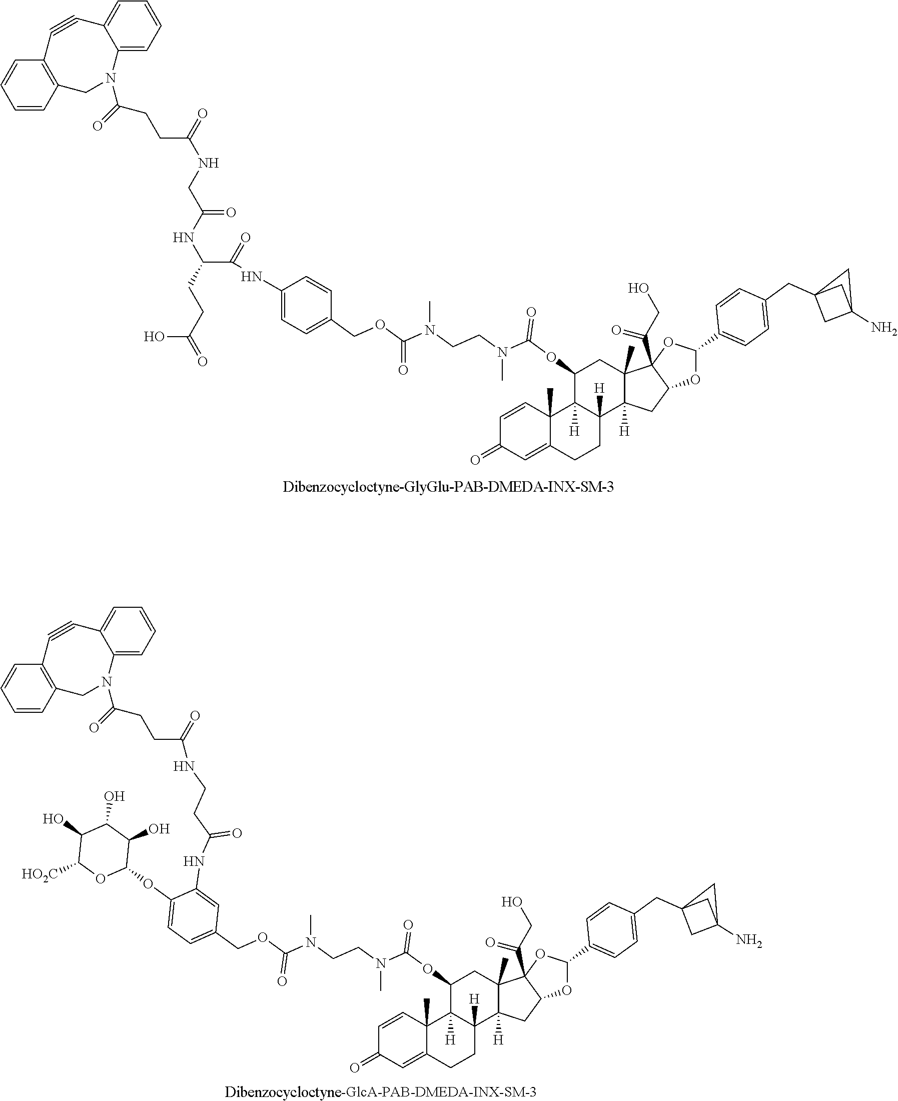

### Tetrazine

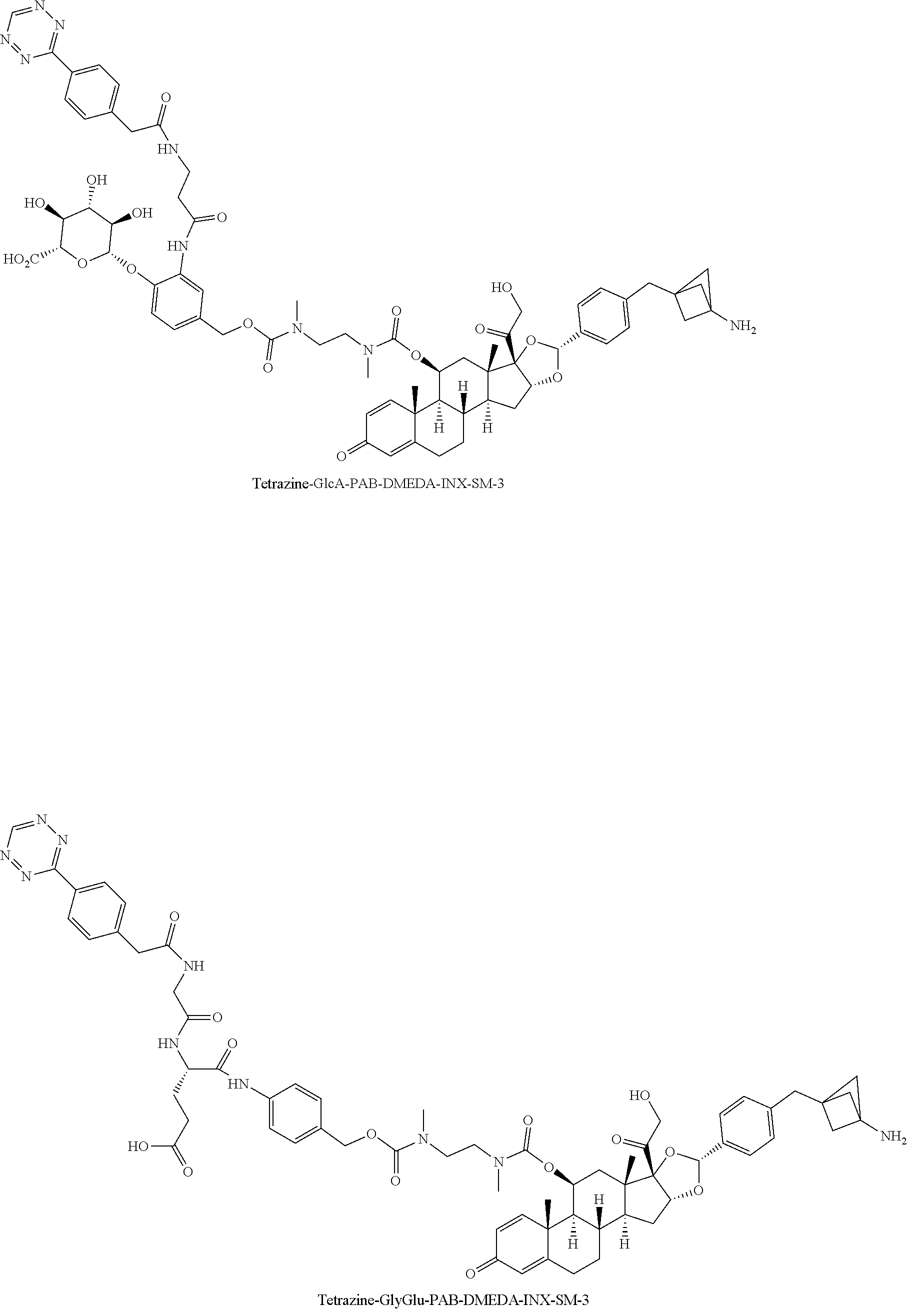

### Amine

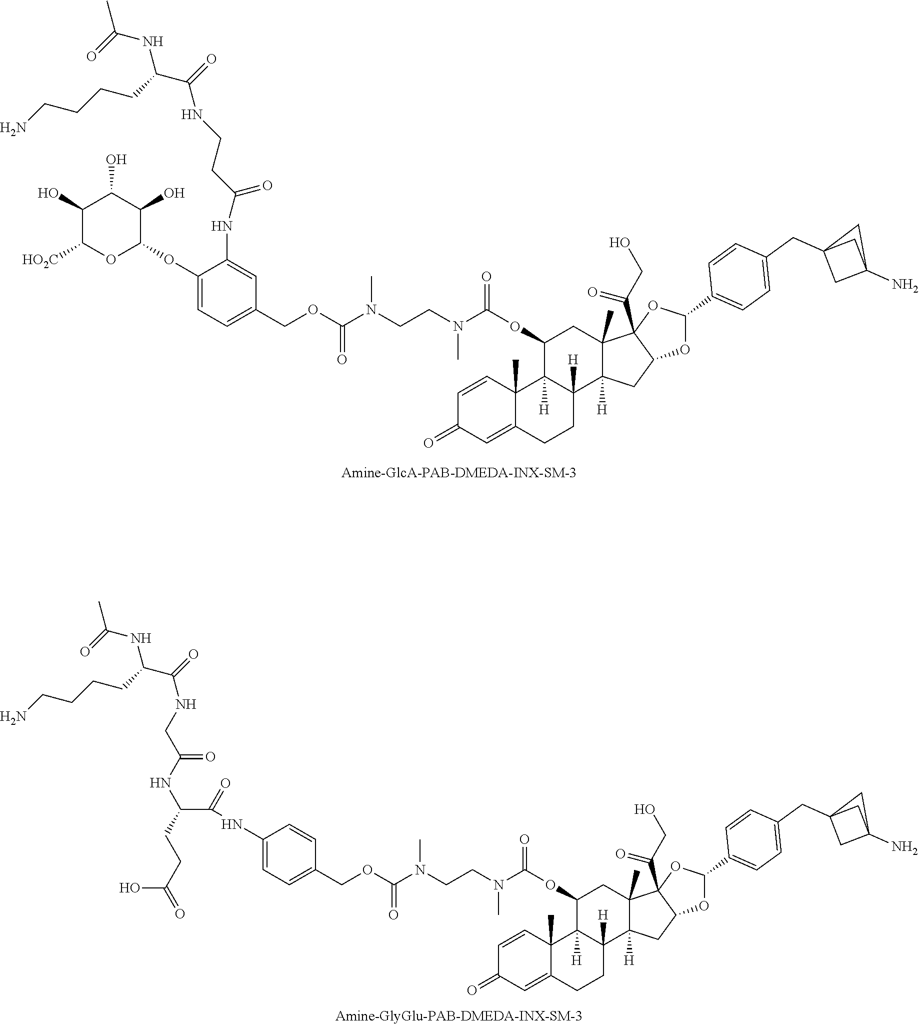

### (IV) Payload-Linker-J

Alternative site of Linker-J attachment to Payload (C17).
INX-SM-3 is used as a payload example

### Alkoxyamine

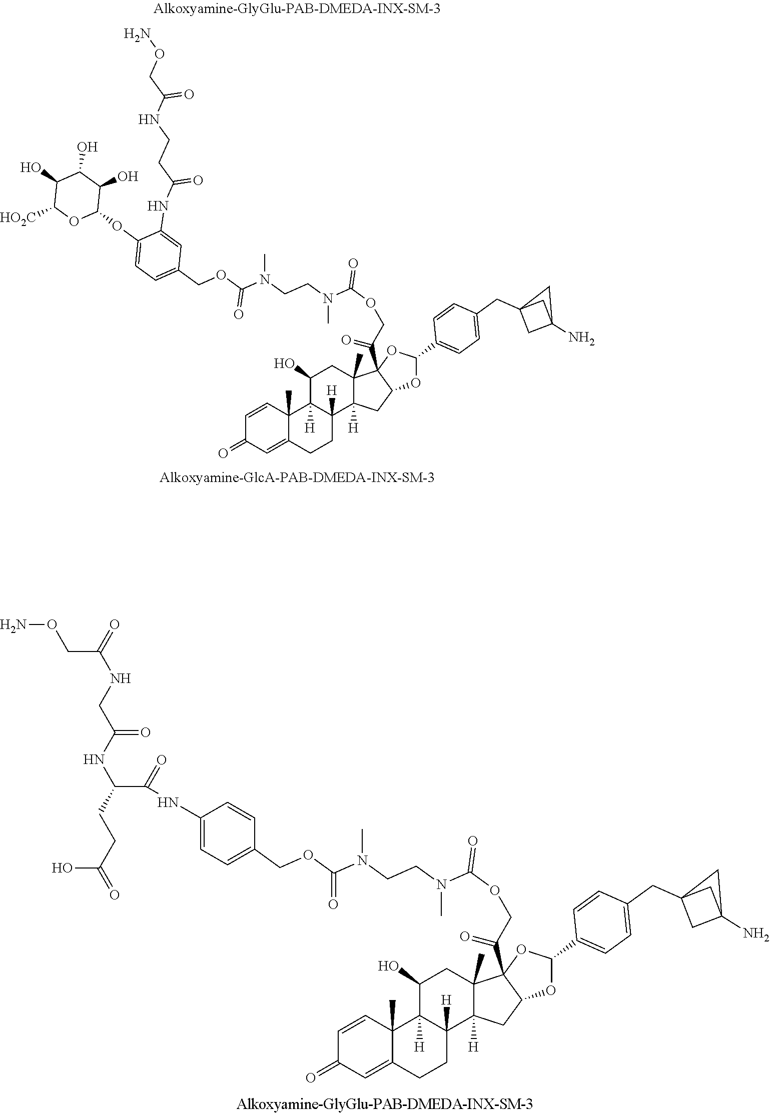

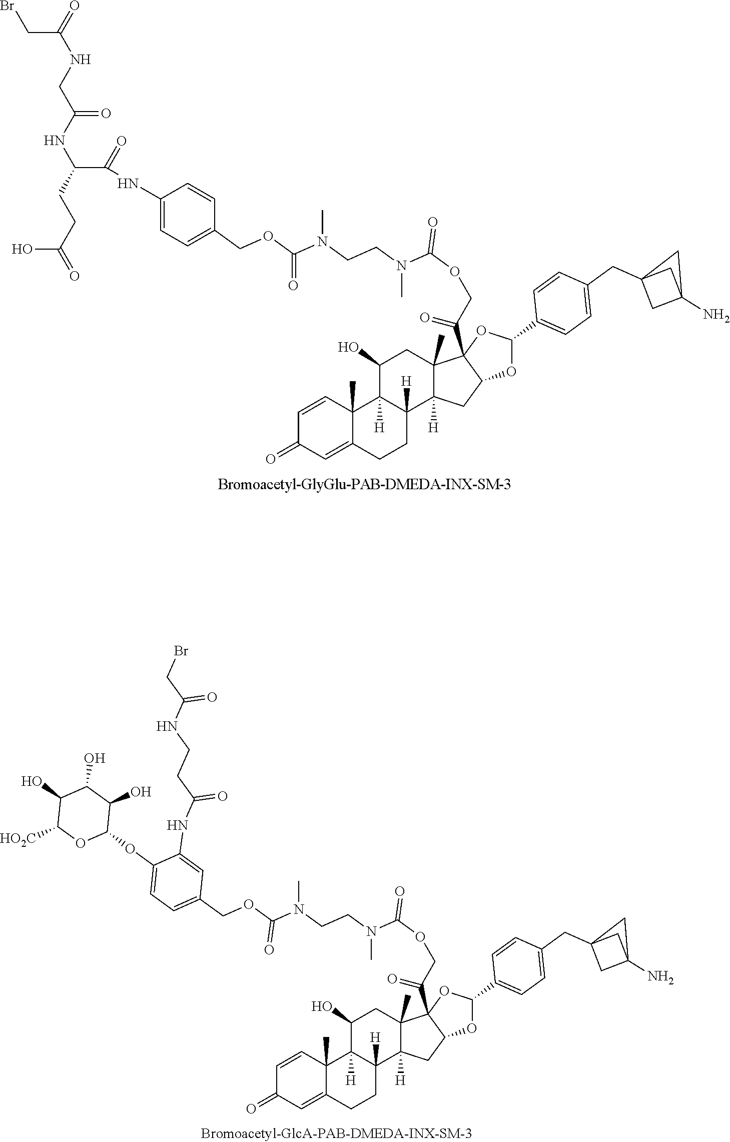

### Maleimide

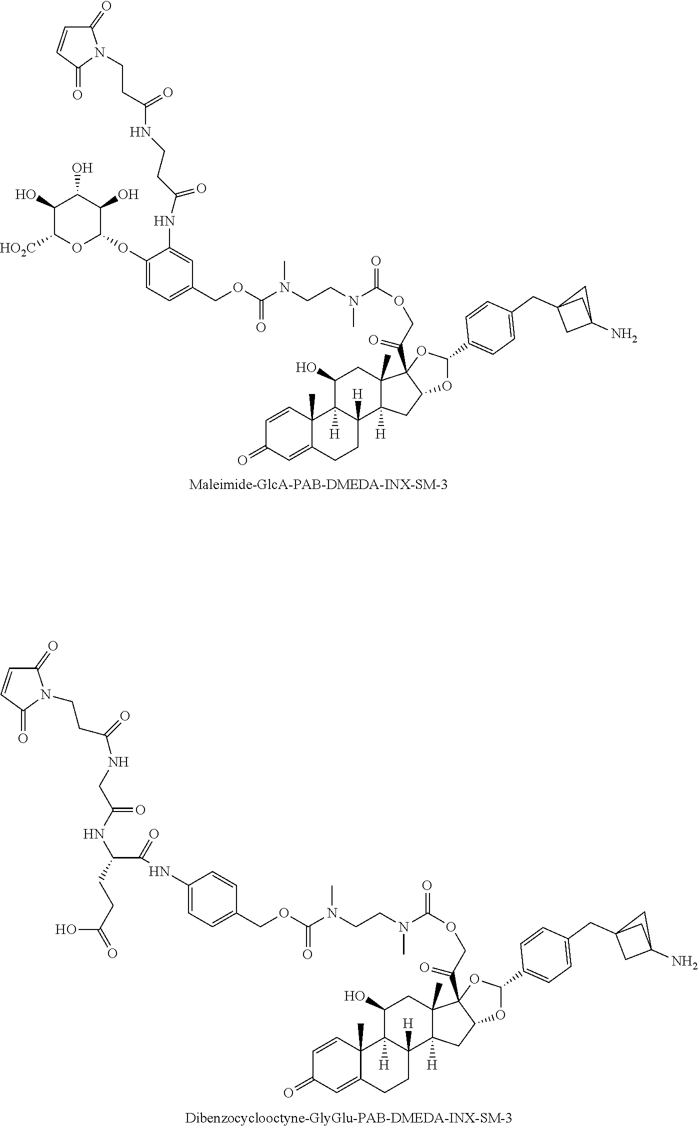

### Dibenzocyclooctyne

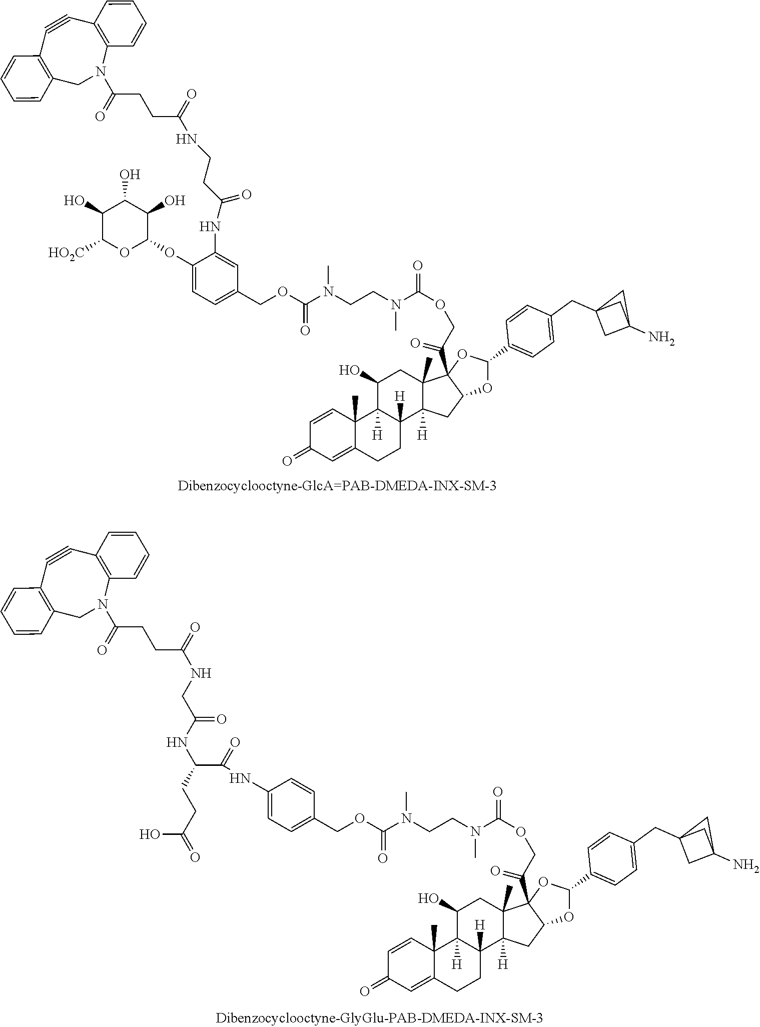

### Tetrazine

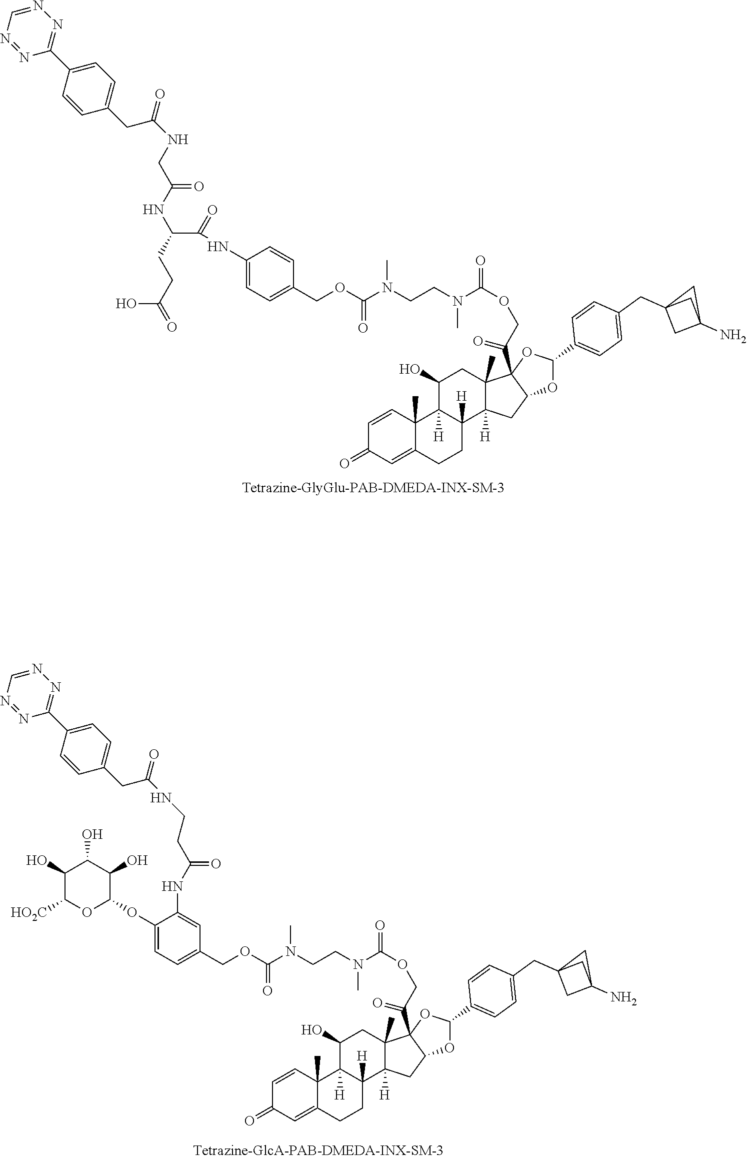

### Amine

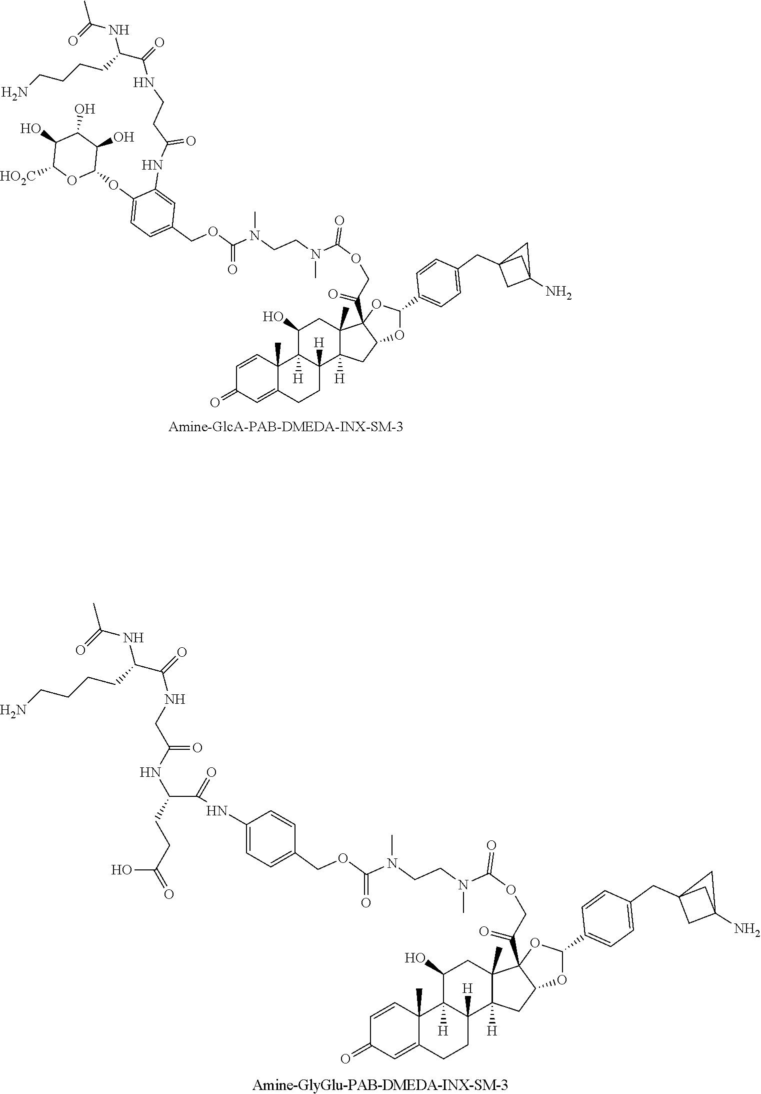

### V. EXEMPLARY PAYLOAD-LINKER-AB CONJUGATES (WHEREIN INX-SM-3 IS AN EXEMPLARY PAYLOAD)

Different ADC conjugates comprising an antibody or antibody fragment that binds to an antigen expressed by an immune cell, optionally an anti-VISTA antibody or fragment having the pH binding/PK properties described herein, one or more linkers and one or more payloads (steroid or other anti-inflammatory compound) may be produced using detailed synthetic methods above-described and as disclosed in the examples. Some exemplary ADCs comprising an exemplary steroid payload (INX-SM-3) are provided below. Other exemplary ADCs are disclosed in the examples and may further comprise other steroid payloads according to the invention, i.e., those comprising a steroid compound of Formula I, II or III as disclosed herein.

### Alkoxyamine+Ketone Conjugation (C11-OH Linked)

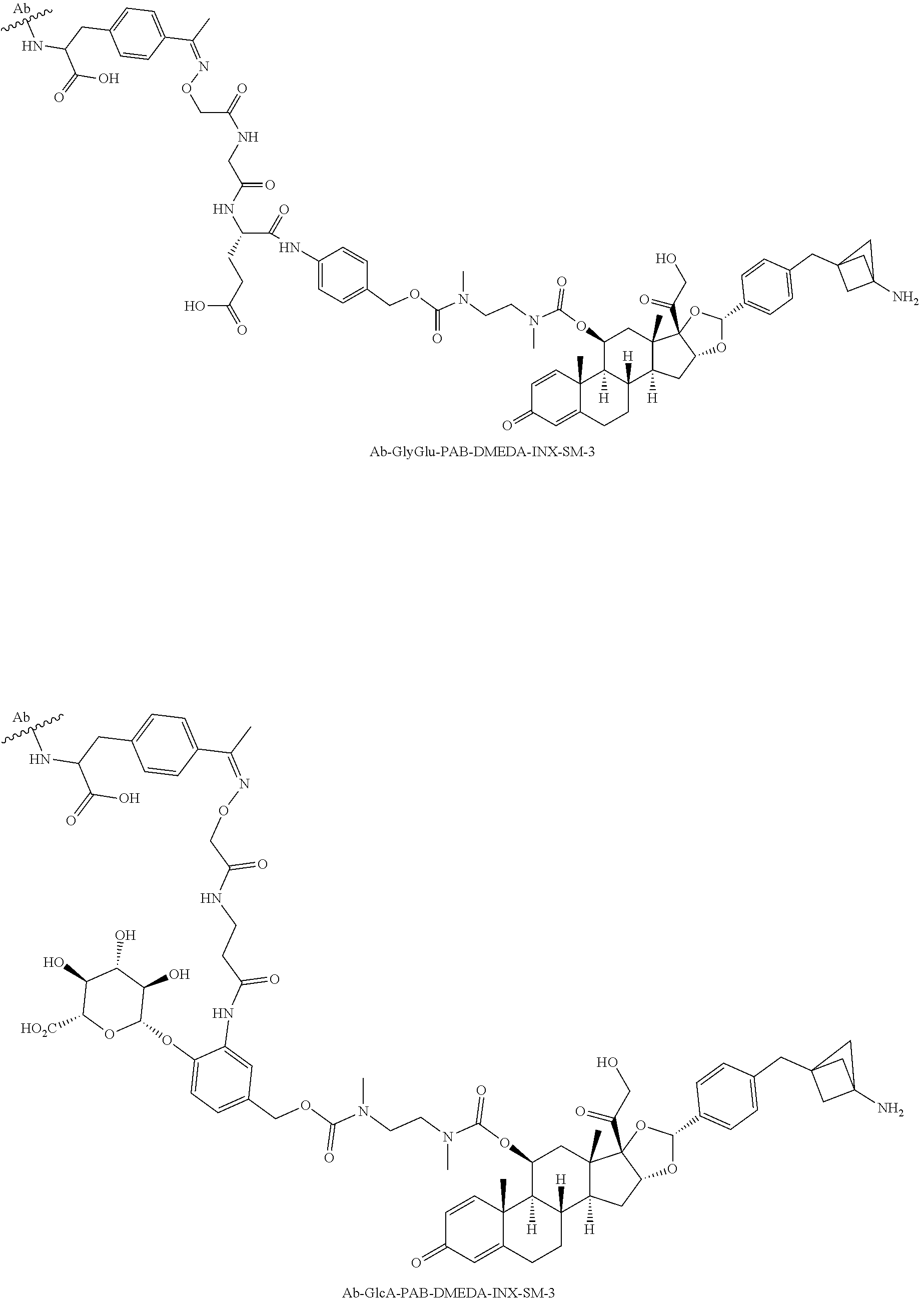

Azide+Dibenzocyclooctyne Conjugation (C11-OH linked)

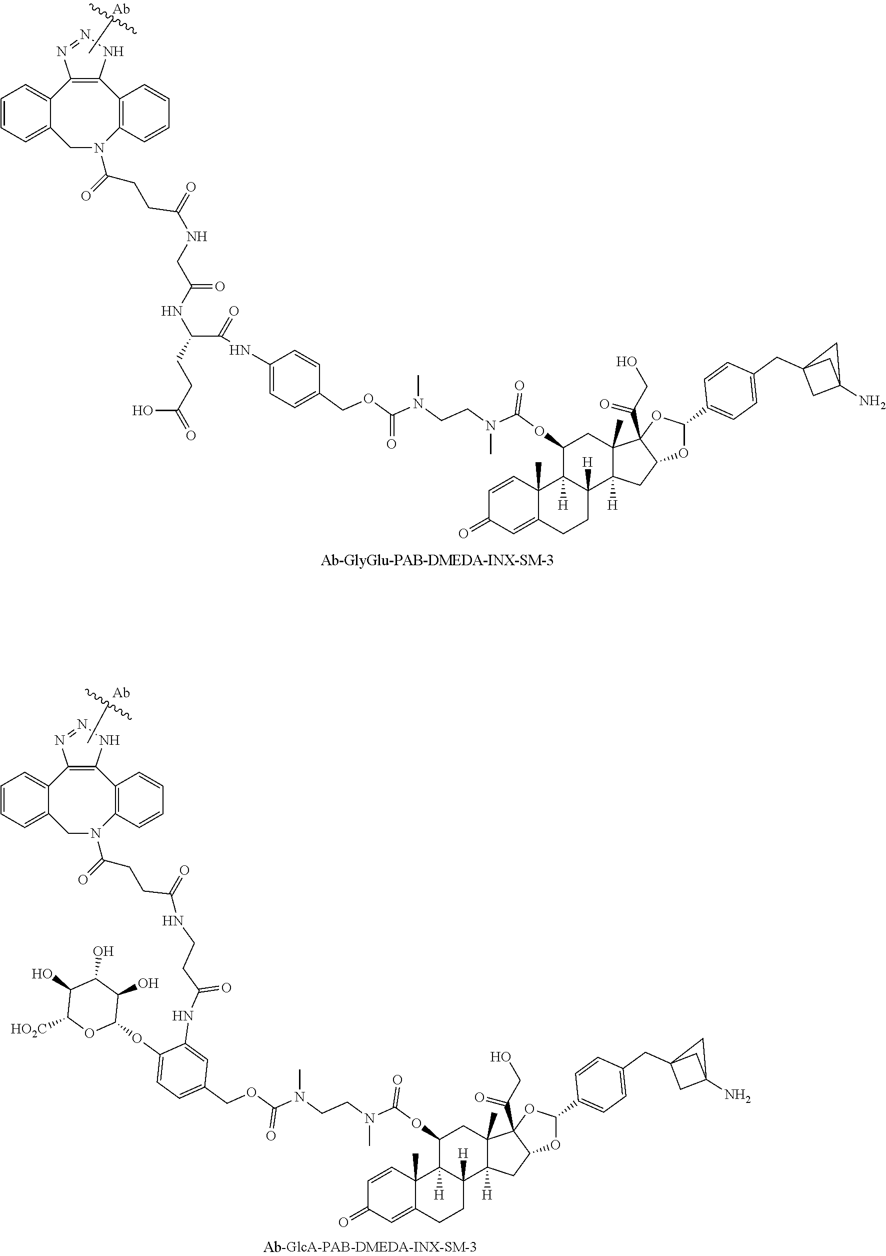

### Haloacetyl+Cysteine Conjugation (C11-OH Linked)

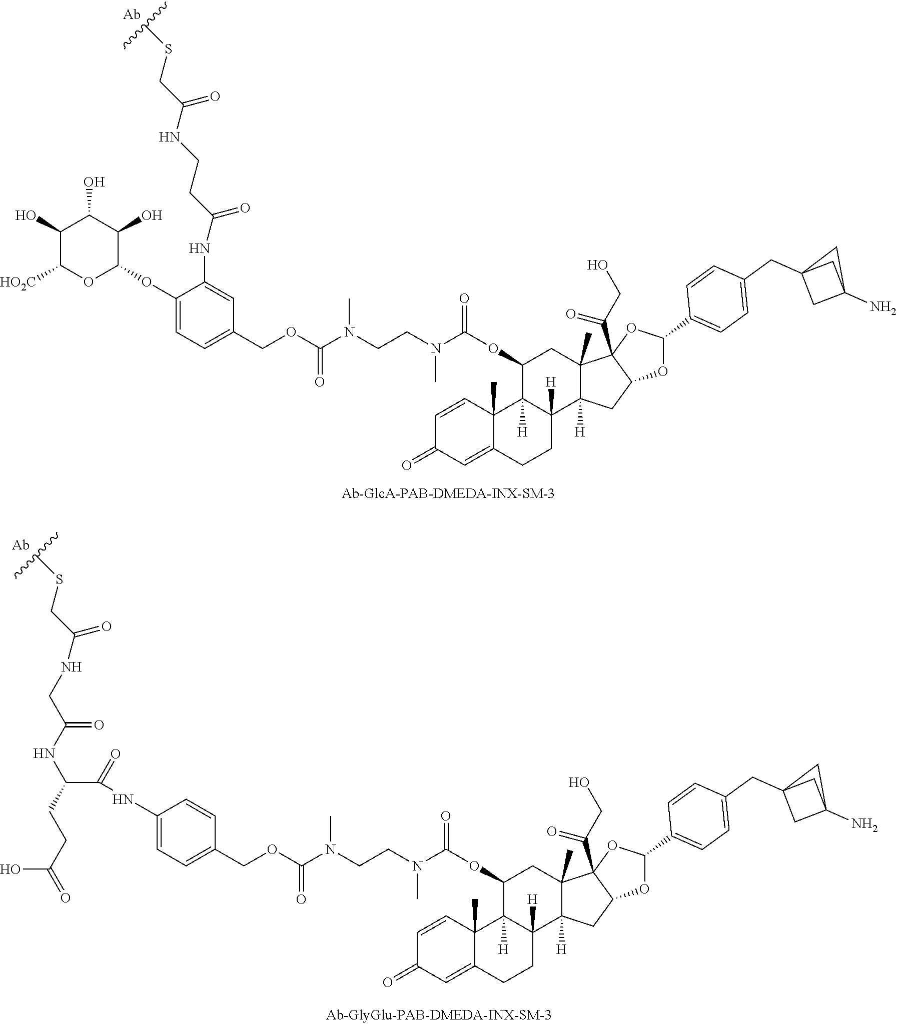

Maleimide+Cysteine Conjugation (C11-OH linked)

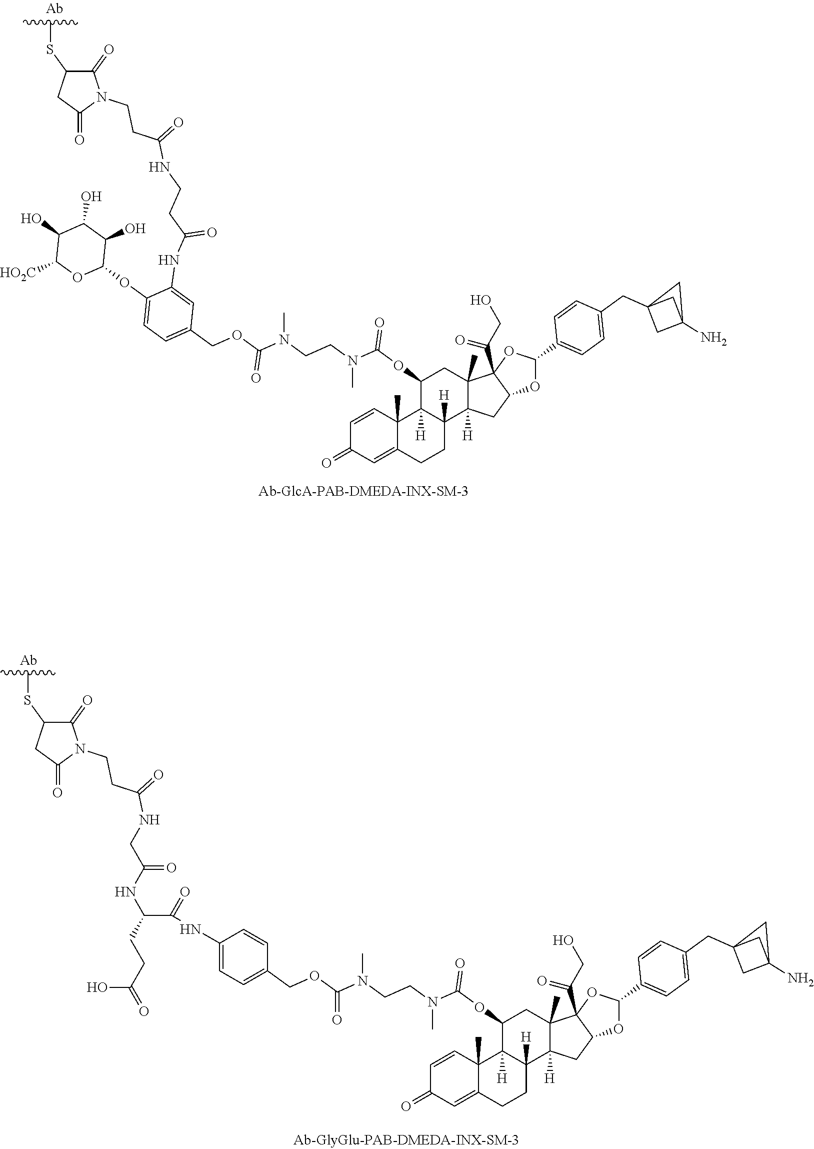

### Tetrazine+Trans-Cyclooctene Conjugation (C11-OH Linked)

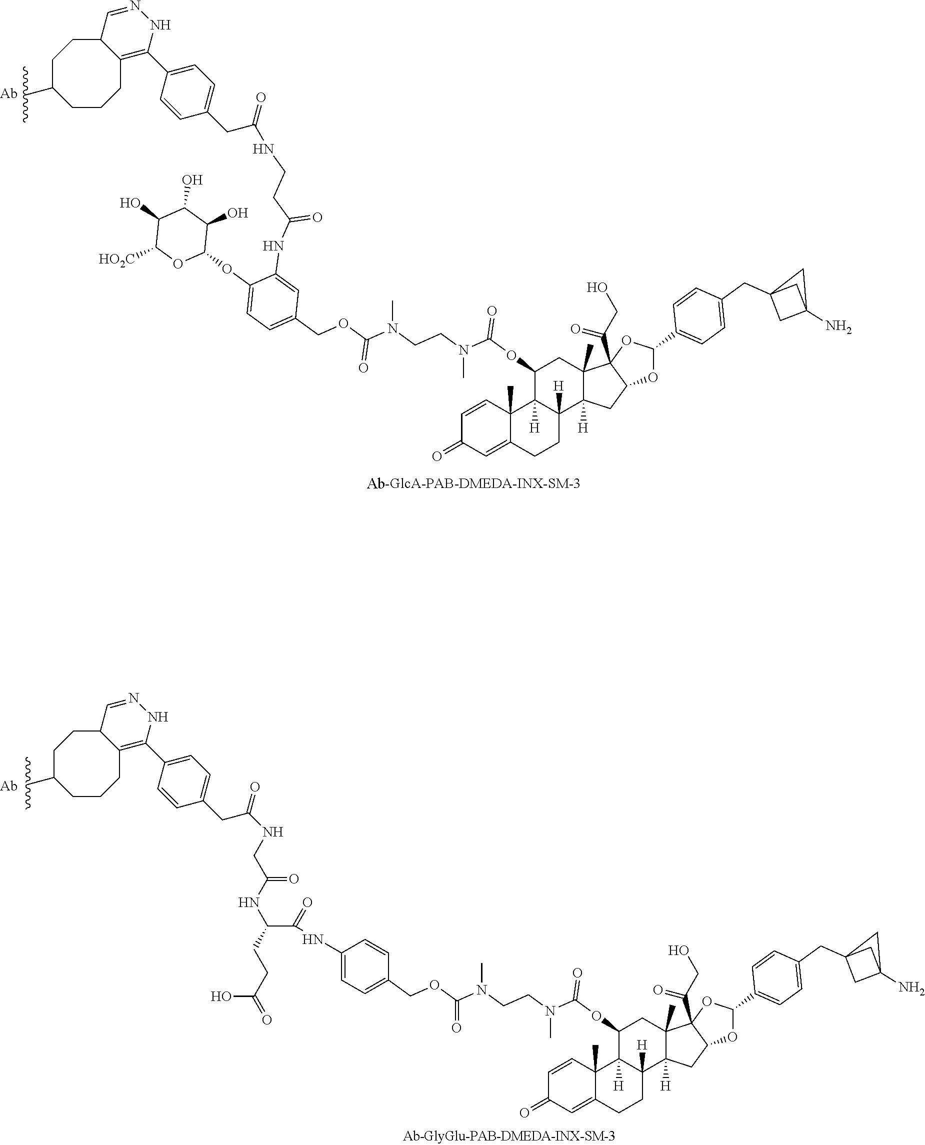

Amine+Glutamine Conjugation using trans glutaminase (C11-OH linked)

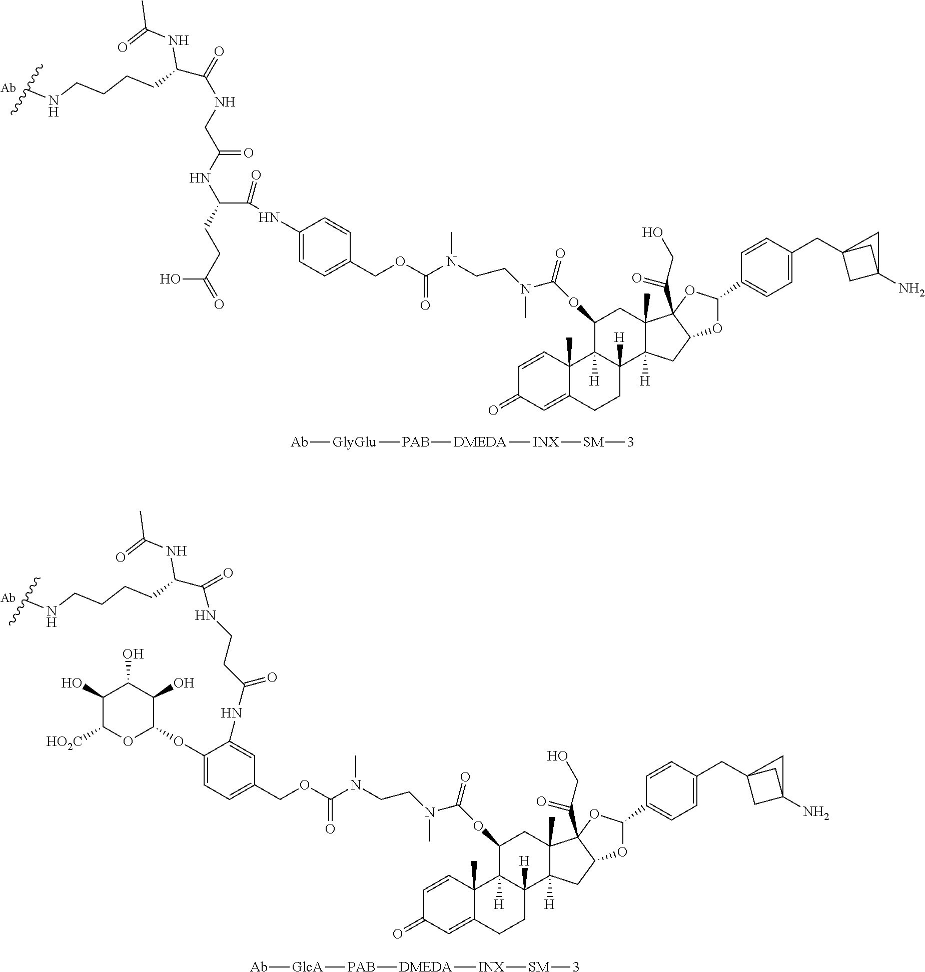

### Alkoxyamine+Ketone Conjugation (C17)

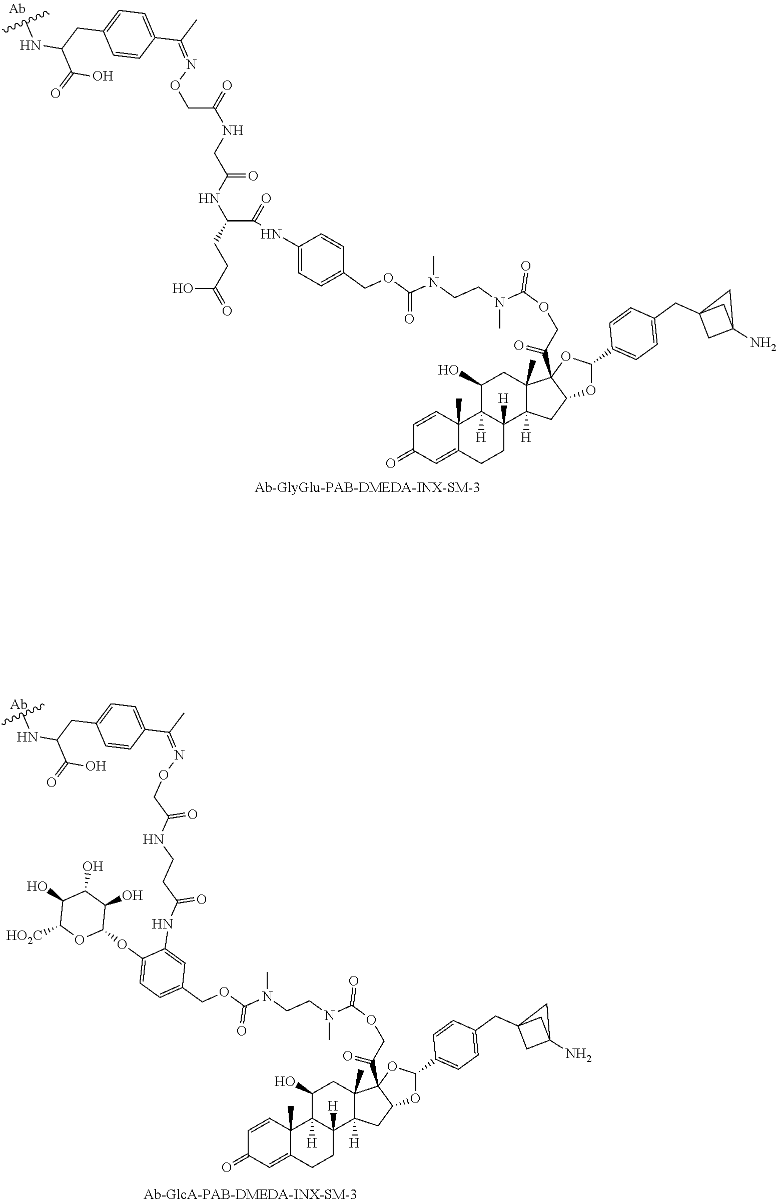

### Azide+Dibenzocyclooctyne Conjugation (C17)

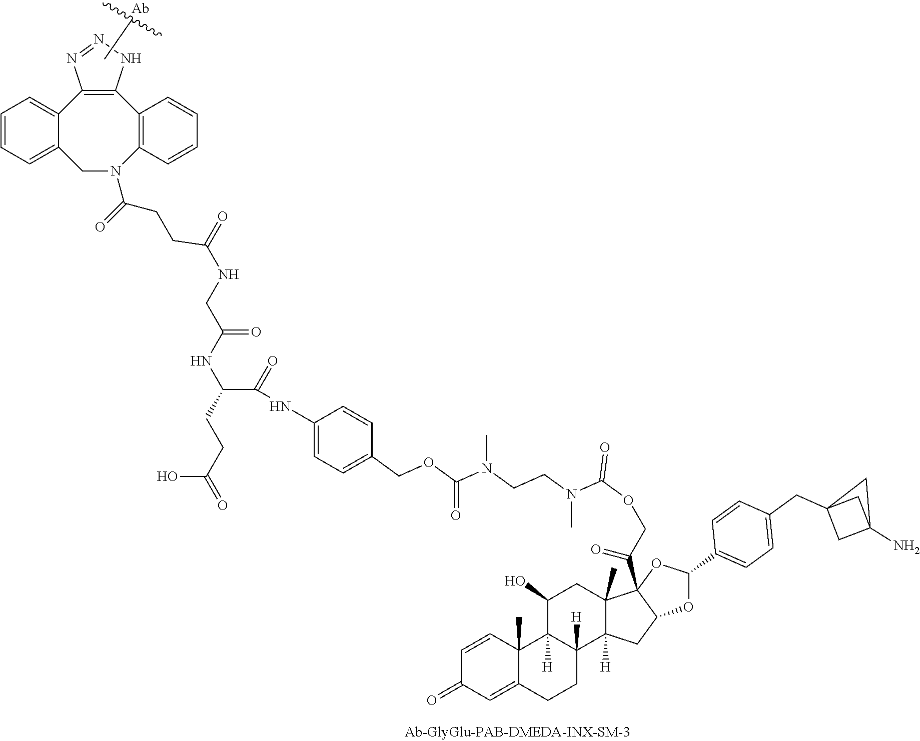

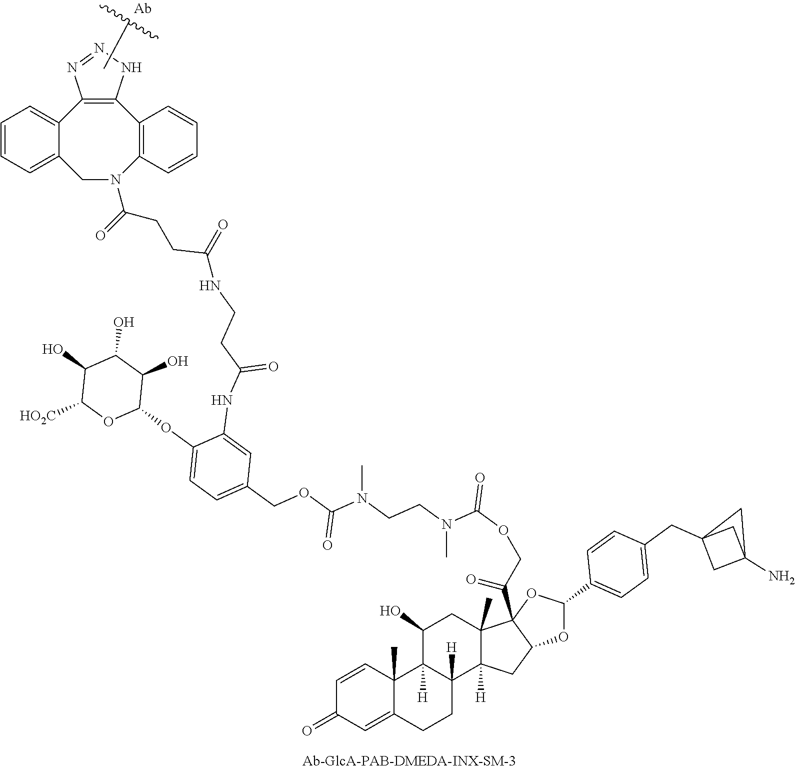

### Haloacetyl Conjugation to Cysteine (C17)

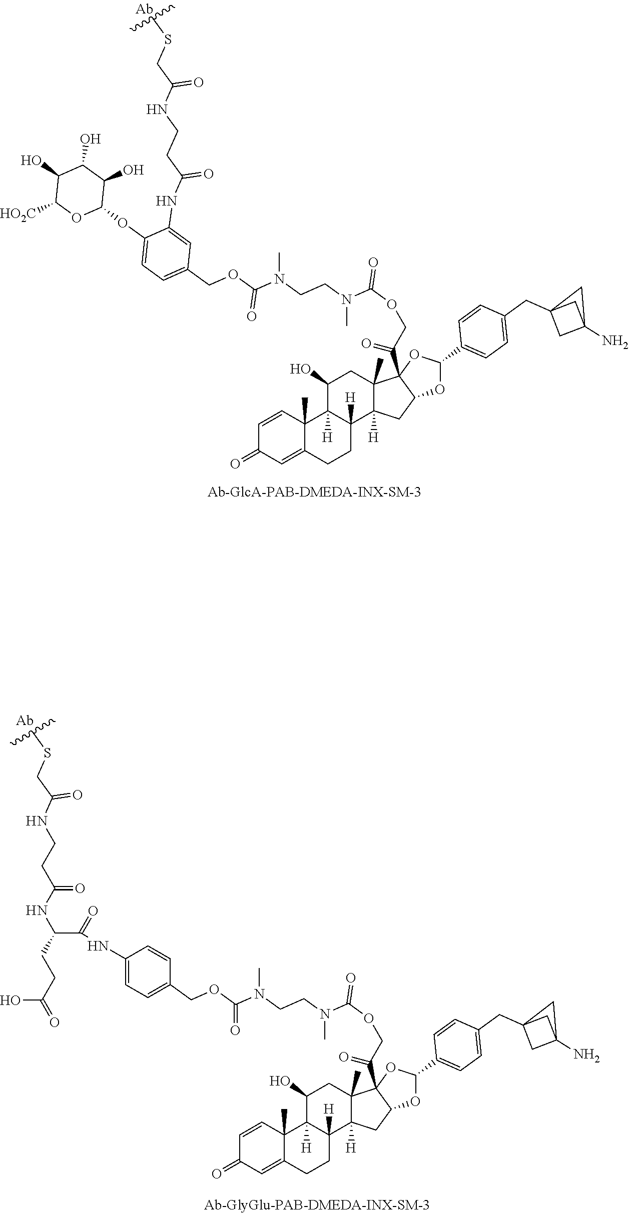

### Maleimide Conjugation to Cysteine (C17)

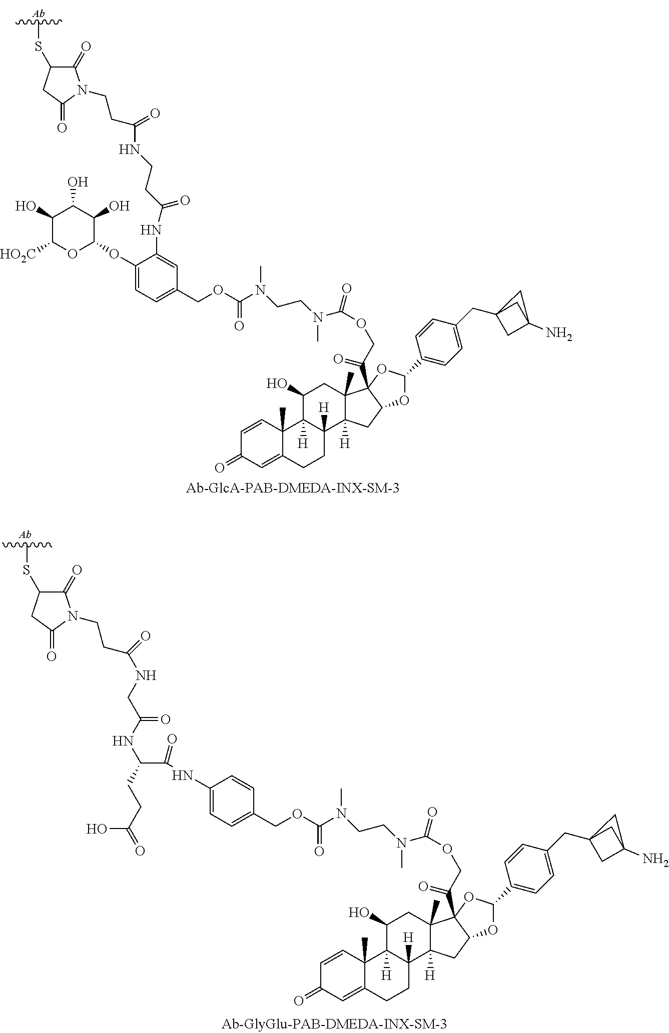

### Tetrazine+Trans-cyclooctene (C17)

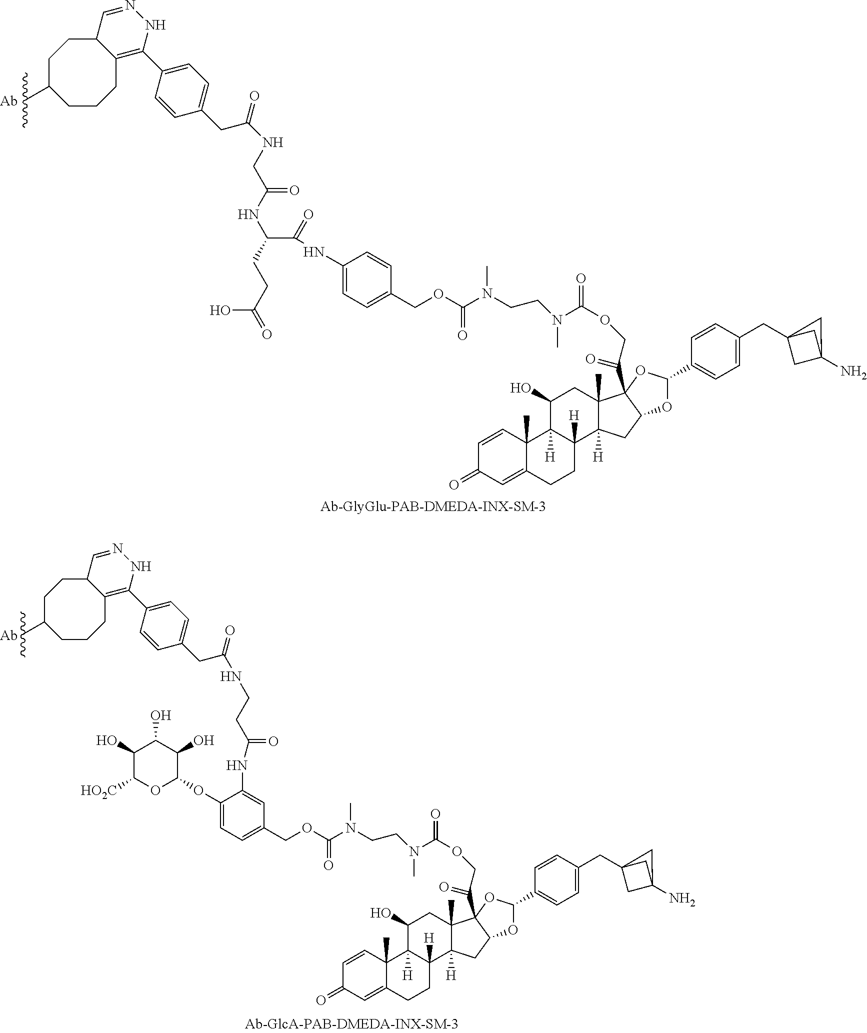

Amine+Glutamine conjugation using trans glutaminase (C17)

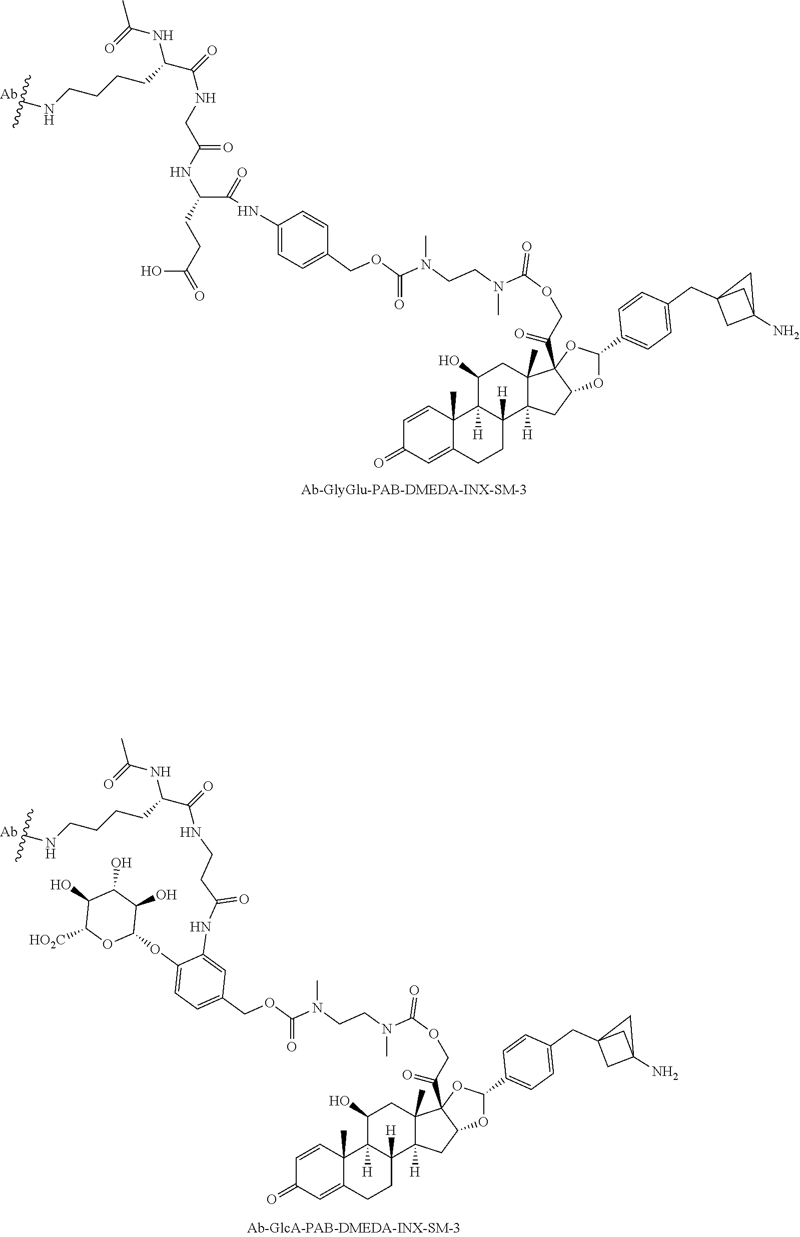

### N-Linked Payload-Linker-Ab ADC's

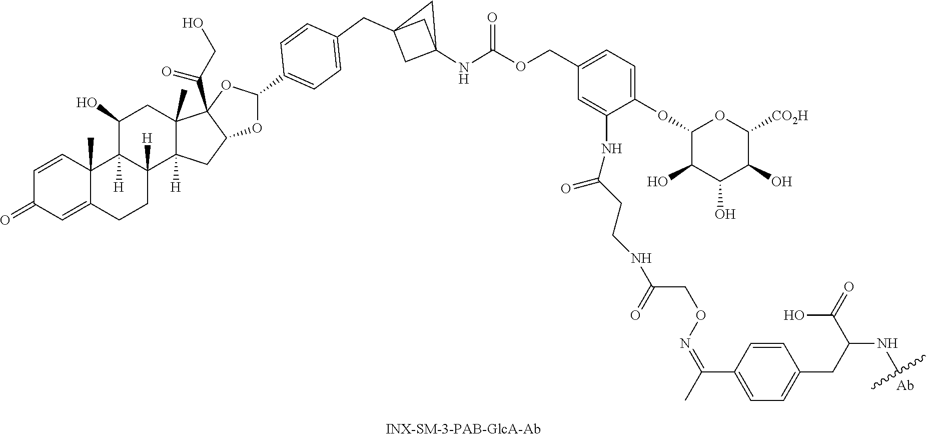

### Axoxyamine: Matsno Conjugation

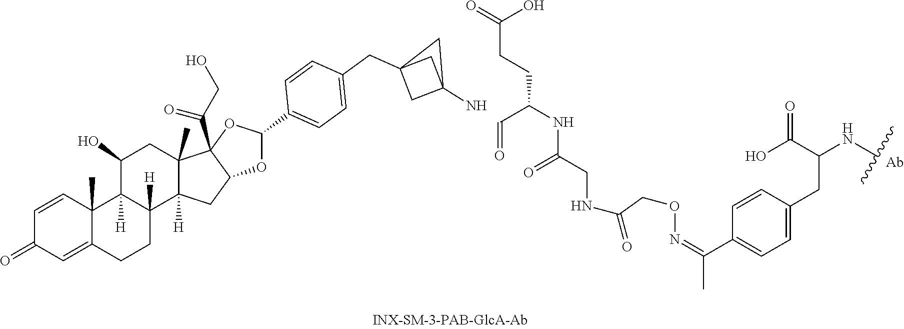

### Alkoxyamine+Ketone Conjugation

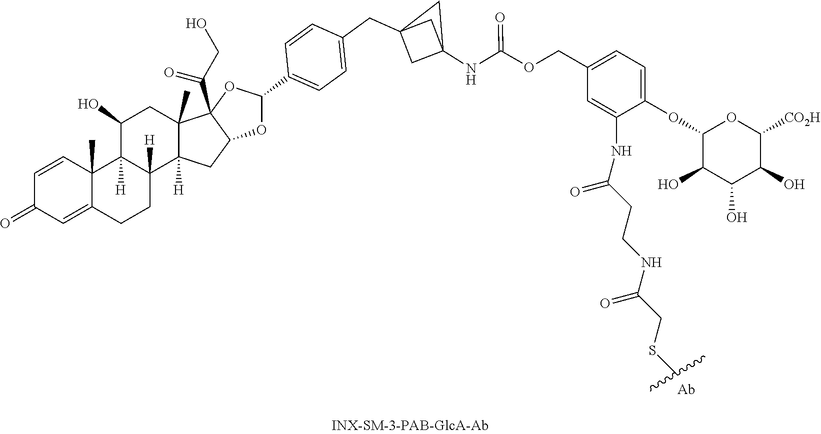

### Haloacetyl Conjunction

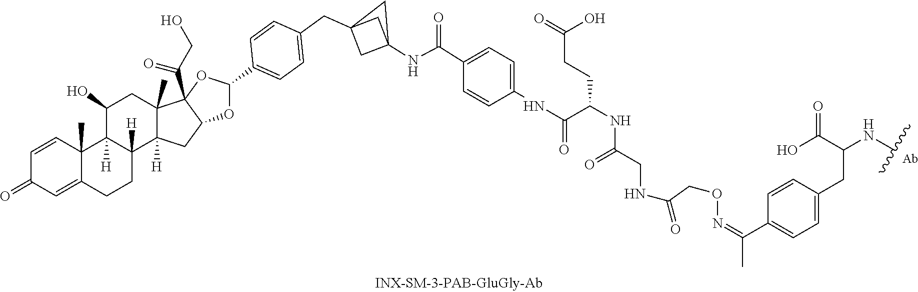

### Haloacetyl Conjunction

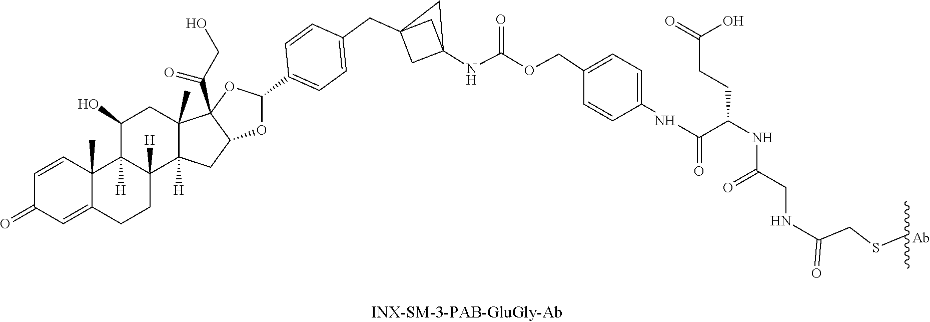

### Azide+Dibenzocyclooctyne Conjugation

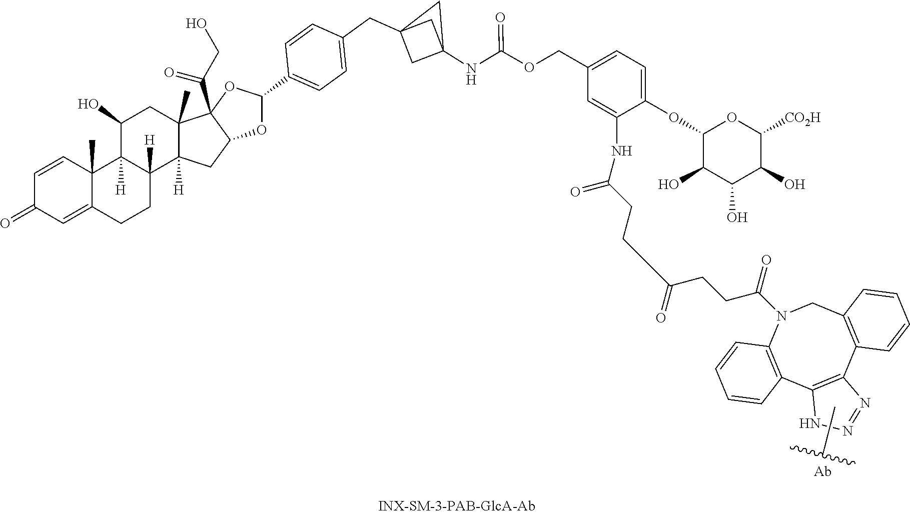

### Azide+Dibenzocyclooctyne Conjunction

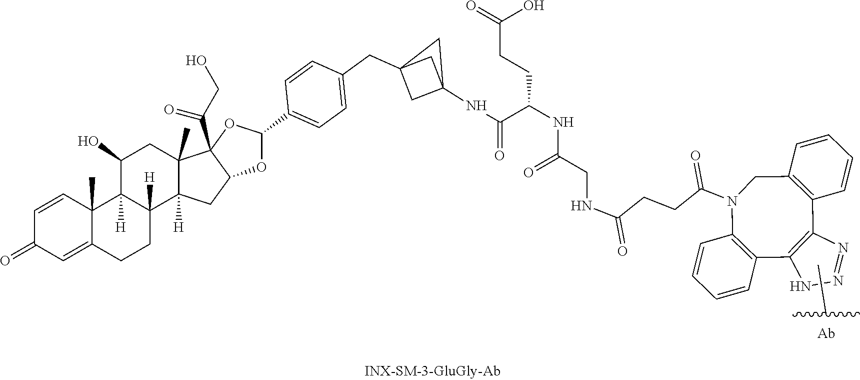

### Azide+Dibenzocyclooctyne Conjunction

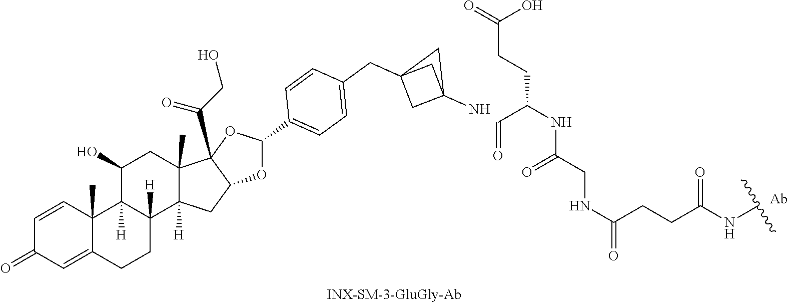

### N-Hydroxysuccinamide Conjunction

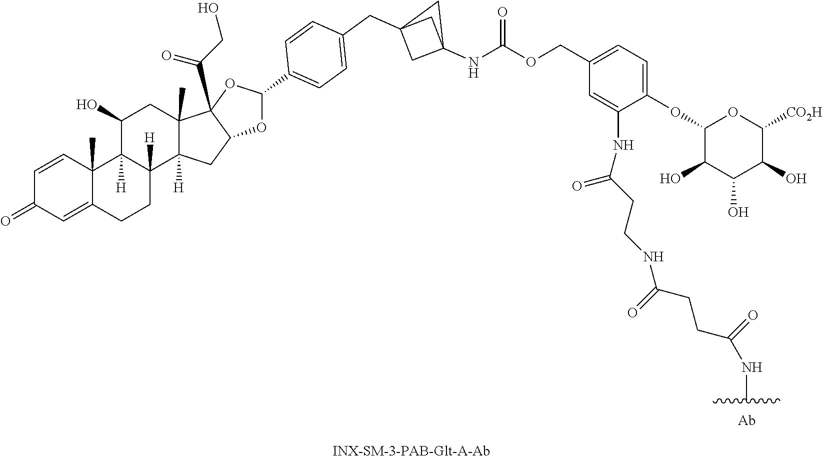

### Azide+Dibenzocyclooctyne Conjunction

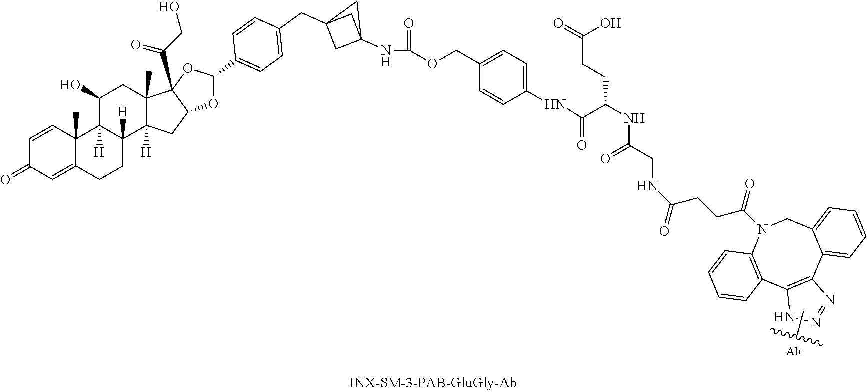

### N-Hydroxysuccinimide Conjugation

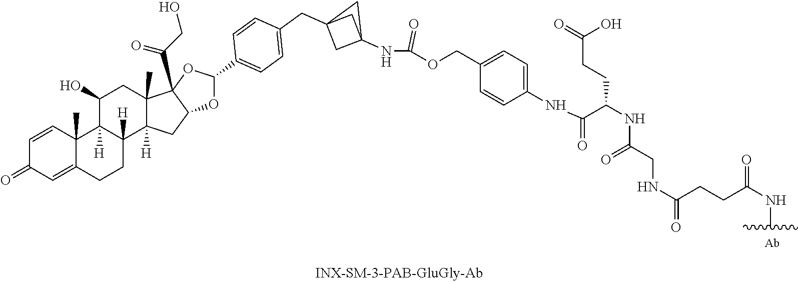

### Maleimide Conjugation

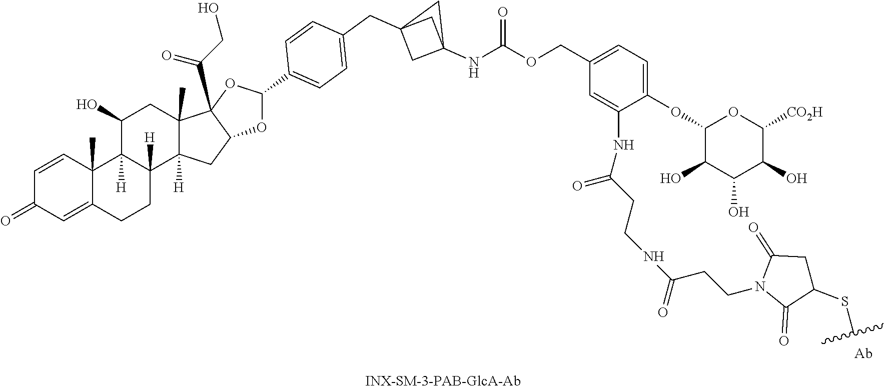

### Maleimide Conjugation

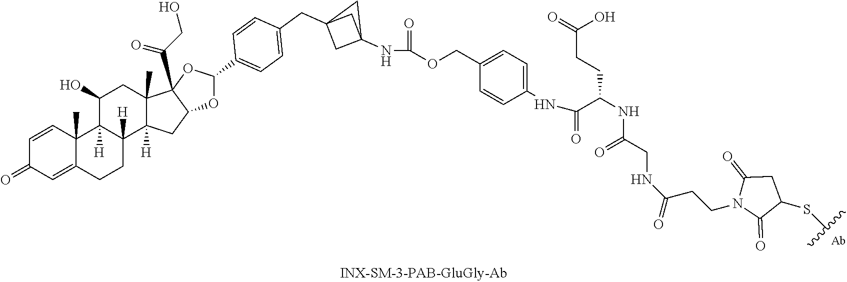

### Maleimide Conjugation

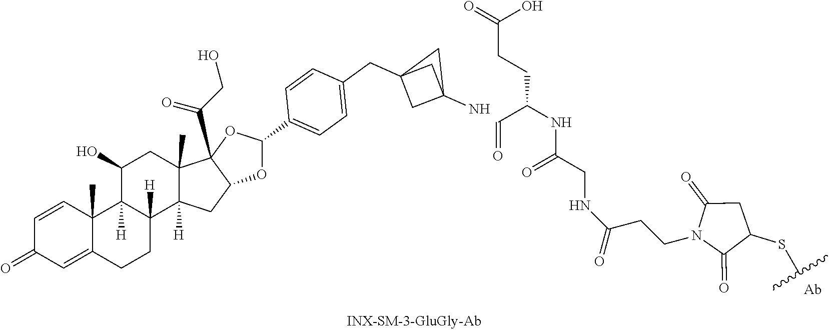

### Trans-cyclodiene+Tetrazine Conjugation

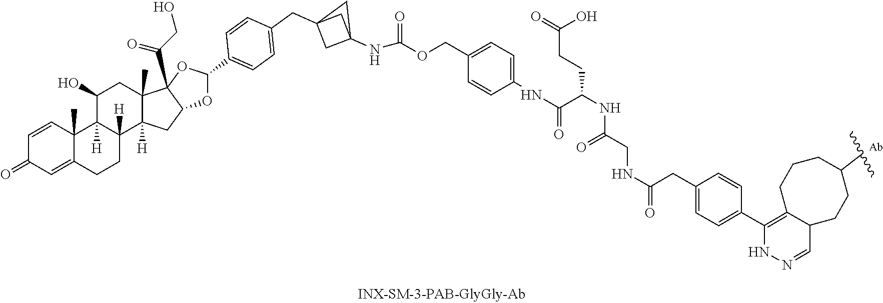

### Trans-cyclooctene+Tetrazine Conjugation

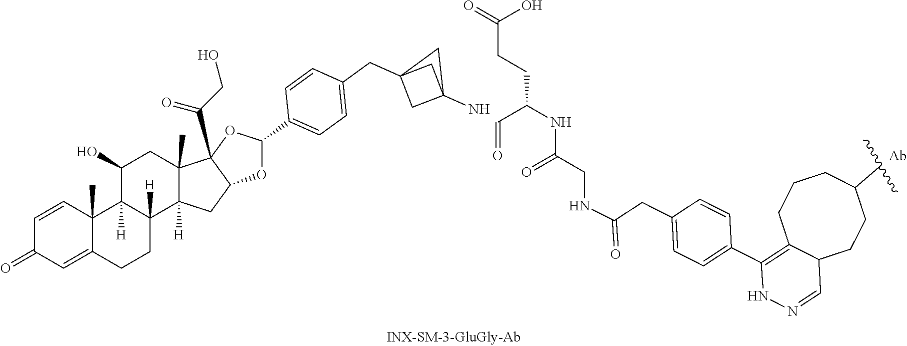

### Trans-cyclooctene+Tetrazine Conjugation

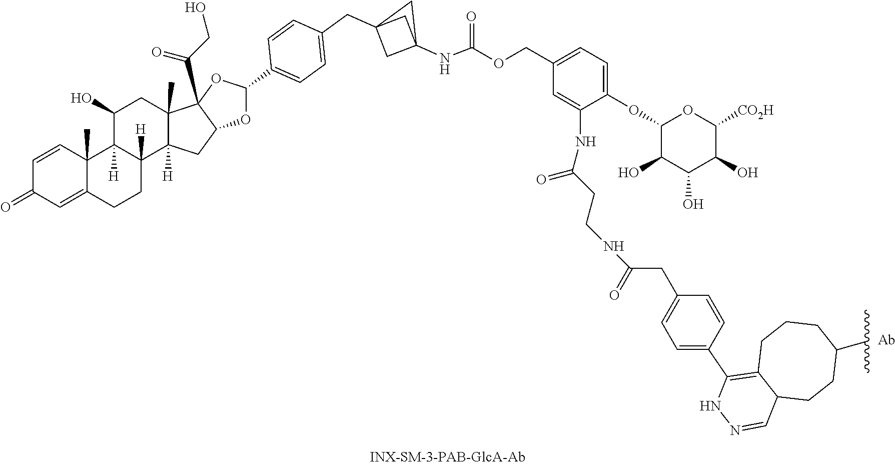

### Amine Conjugation

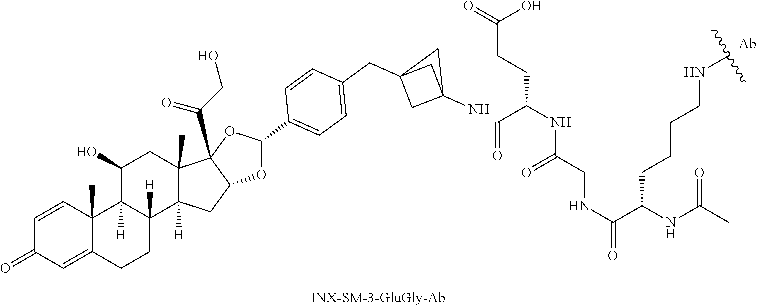

### Amine Conjugation

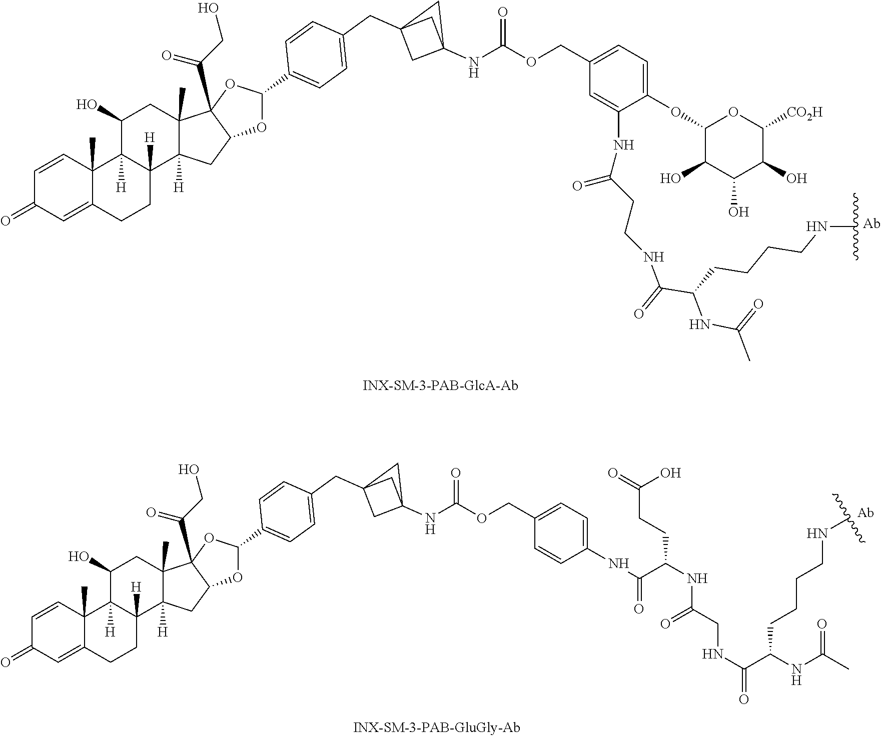

### Haloacetyl Conjugation

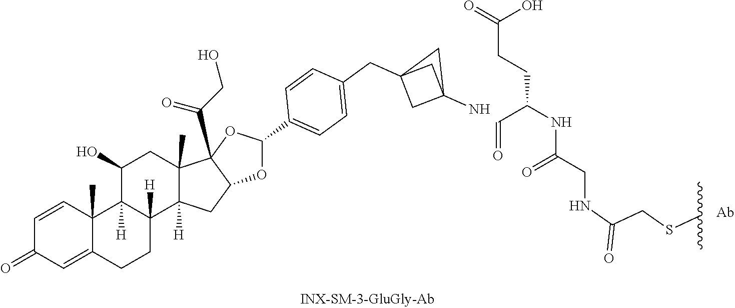

### Therapeutic Applications of Steroid Payloads of Formula I, II and III, Steroid-Linker Payloads and ADCs Containing

ADCs which comprise a synthetic glucocorticoid agonist according to the invention may be produced as above-described and in the examples infra. In some exemplary embodiments the antibody contained therein will comprise an anti-human VISTA antibody or fragment which binds to immune cells at physiologic pH and which moreover possesses a short PK or may comprise an antibody or antibody fragment that binds to another immune cell antigen, preferably an antibody or antibody fragment which efficiently internalizes target immune cells and preferably which does not substantially bind to or internalize non-target cells. Generally the subject ADCs will comprise a steroid of Formula I, II or III or steroid-linker containing same and an antibody or antibody fragment which binds to an antigen expressed on specific types of immune cells, preferably an antigen that is only expressed on target immune cells, or which is expressed at substantially higher levels on target immune cells compared to non-target cells which express the antigen, e.g., at least 2-fold, 4-fold, 10-fold higher or greater.

These ADCs may be used for the prophylactic and/or therapeutic treatment of inflammation, allergy, autoimmunity, and diseases associated with inflammation, autoimmunity or allergy including by way of example autoimmune disorders, inflammatory disorders, allergic disorders and cancer conditions disclosed herein. Again, a preferred application of the subject ADCs including those which comprise a steroid of Formula I, II or Ill is for the treatment of chronic diseases associated with inflammation or autoimmunity.

As shown herein, the subject VISTA targeting ADCs, notwithstanding the short pK of the anti-VISTA antibody which is comprised therein which binds to VISTA expressing cells at physiological conditions and which is not engineered to alter or optimize pH binding, i.e., typically around 2.3 days or less in cyno and no more than about 70 hours, no more than about 60 hours, no more than 50 hours, no more than 40 hours, no more than 30 hours, no more than 24 hours, no more than 22-24 hours, no more than 20-22 hours, no more than 18-20 hours, no more than 16-18 hours, no more than 14-16 hours, no more than 12-14 hours, no more than 10-12 hours, no more than 8-10 hours, no more than 6-8 hours, no more than 4-6 hours, no more than 2-4 hours, no more than 1-2 hours, no more than 0.5 to 1.0 hours, or no more than 0.1-0.5 hours in human VISTA engineered mice, has been found to maintain potency for a prolonged period (PD) relative to the much shorter half-life (PK) of the antibody.

As is shown herein ADC conjugates according to the invention which comprise such anti-VISTA antibodies or antibody fragments when evaluated in vivo models have been demonstrated to provide for PD/PK ratios of at least 14:1 and 28:1. Again while Applicant does not want to be bound by their belief, it is theorized that the subject ADCs which comprise such anti-VISTA antibodies or antibody fragments are delivered in very high amounts in target VISTA expressing cells such as macrophages which have long cell turnovers (weeks, months or longer). Essentially, it appears that a depot effect may be created, i.e., a large quantity of the subject ADCs are internalized into VISTA expressing immune cells, i.e., because of very high expression of VISTA whereupon the ADCs are slowly metabolized or cleaved e.g., by cell enzymes resulting in the gradual and prolonged release of therapeutically effective amounts of the steroid payload within the immune cell. However, it should be emphasized that ADCs which comprise steroid payloads according to the invention, i.e., of Formula I, II or III, may comprise antibodies or antibody fragments which bind to other antigens, typically antigens expressed on human immune cells. In fact in the examples Applicant shows that ADCs comprising steroid payloads according to the invention which comprise antibodies which target different immune cell antigens also possess desired potency properties.

Having described the invention, the following examples are provided to further illustrate the invention and its inherent advantages.

### Examples

The following examples describe exemplary embodiments of the invention.

### Abbreviations Used in Examples

Ab Antibody
AF488 Alexa Fluor 488
ADC antibody drug conjugate
BSA bovine serum albumin, V fraction
CD14 Monocyte differentiation antigen CD14
CD20 T-Cell Surface Glycoprotein CD4
CD4 Differentiation antigen CD20
CD8 T-Cell Surface Glycoprotein CD8
CD66b Granulocyte GPI-linked glycoprotein
SSC side scatter
CD25 IL-2R a chain
CD127 IL-7 receptor a chain
ConA Concanavalin A
CPT Citric acid/phosphate with 0.05% Tween 20
CPTB: Citric acid/phosphate with 0.05% Tween 20 and 1% BSA
DAR drug antibody ratio
Dex Dexamethasone
ECD Extracellular domain
FA formaldehyde fluorescence activated cell sorting
FACS FBS fetal bovine serum
Fc heavy chain constant region (hinge/CH2/CH3) of an antibody
FMO Fluoresence Minus One Control (Experimental cells stained with all the fluorophores minus one fluorophore)
GC Glucocorticoid
h hour
HIC Hydrophobic interaction chromatography
HMW High molecular weight
i.p. intraperitoneal
i.v. Intravenous
KI knock-in
LAL Limulus Amebocyte Lysate
LOD limit of detection
LOQ limit of quantitation
LPS Lipopolysaccharides
M molar concentration
mAb monoclonal antibody
MFI median fluorescence intensity
min minute
MS Mass spectrometry
mTNFα membrane tumor necrosis factor alpha
pAb polyclonal antibody
PBS phosphate buffered saline
PBMCS peripheral blood mononuclear cells
PBS phosphate buffered saline
PD Pharmacodynamic
PK Pharmacokinetic
PRM Peritoneal resident macrophages
PT PBS with 0.05% Tween 20
PTB PBS with 0.05% Tween 20 and 1% BSA
PTS Portable Test System
QC Quality control
RP-HPLC
Reverse Phase-High Pressure Liquid Chromatography
RPMI RPMI 1640, basal culture medium
RSV respiratory syncytial virus
RT Room temperature
SEC Size exclusion chromatography
SPR Surface plasmon resonance
SSC side scatter
TMDD Target mediated drug disposition
WB Whole Blood

### Example 1: Synthesis and Characterization of Exemplary Steroid-Anti-VISTA Antibody Conjugates

### A. Synthesis

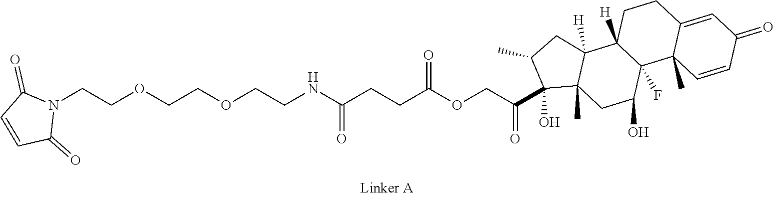

### Scheme for Synthesis of Linker A

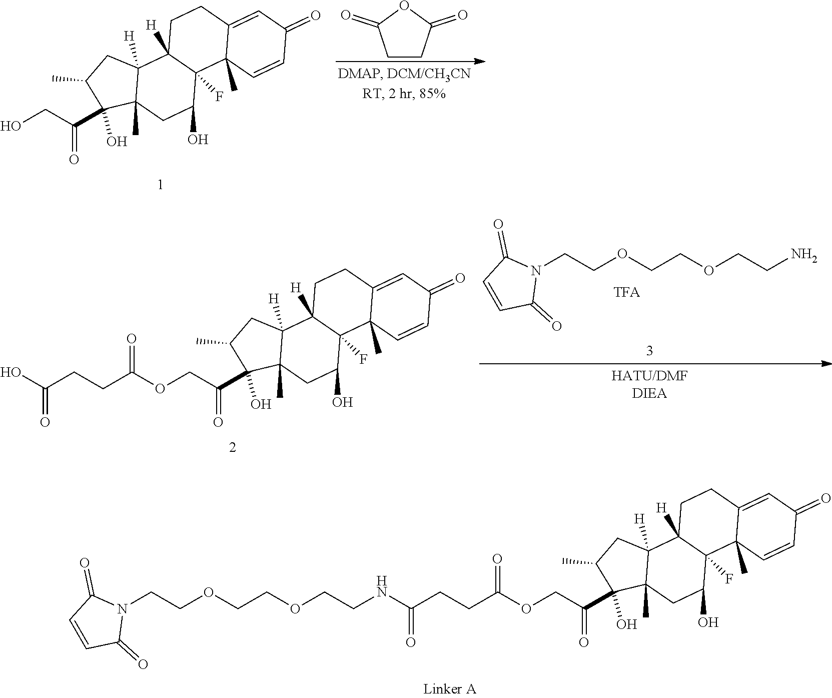

### Procedure

### General Procedure for the Preparation of Compound 2

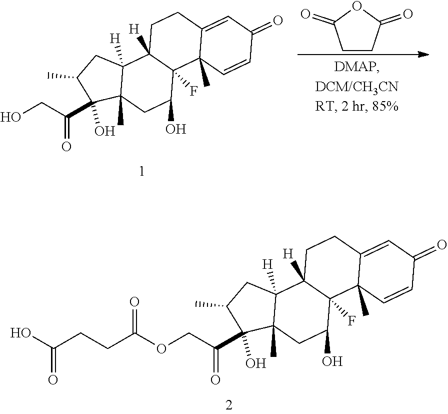

To a solution of compound 1 (3.0 g, 7.64 mmol, 1.0 eq) in a dichloromethane/acetonitrile (500 mL/100 mL) were added cyclic anhydride (3.0 g, 30.58 mmol, 4.0 eq) and DMAP (1.8 g, 15.29 mmol, 2.0 eq). The reaction mixture was allowed to stir at rt for 2 h and the mixture was concentrated under reduced pressure. The residue was purified by column chromatography on silica gel eluted with DCM/MeOH (10% to 15%)+0.1% AcOH to afford the compound 2 (3.2 g, 85%) as white solid.

TLC: DCM/MeOH=10:1, UV

R_{f} (Compound 1)=0.45

R_{f} (Compound 2)=0.30

LC-MS: 394.40 (M+1)

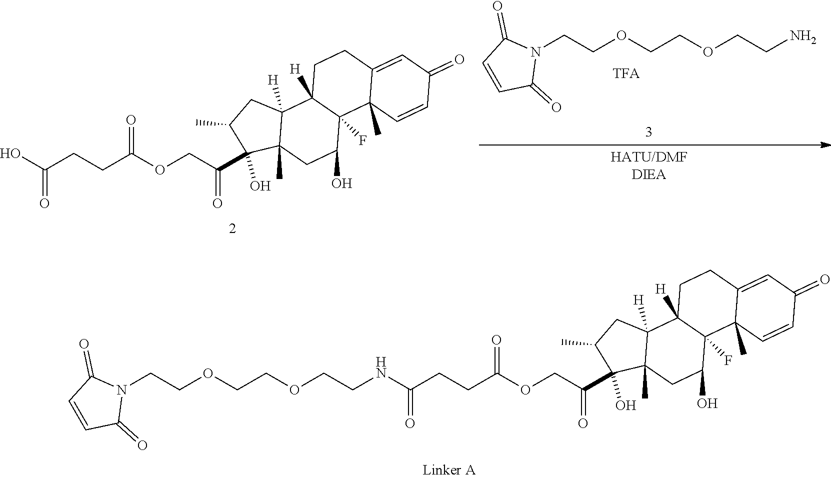

To a solution of 2 (220 mg, 0.45 mmol) and 3 (230 mg, 0.67 mmol) in NMP (4 mL) was added HATU (342 mg, 0.90 mmol) and DIPEA (232 mg, 1.8 mmol). The mixture was stirred at rt for 5 h. The mixture was purified by prep-HPLC (ACN/H2O, 0.1% HCOOH) to give Linker A (122 mg, 39%).

LCMS: 703 [M+H],

¹H NMR (CDCl₃, 300 MHz) (δ, ppm) 7.20 (d, J=9.0 Hz, 1H), 6.73 (s, 2H), 6.52 (br, 1H), 6.33 (d, J=9.0 Hz, 1H), 6.11 (s, 1H), 4.91 (q, J=17.3 Hz, 2H), 4.35 (d=9.3 Hz, 1H), 3.76-3.42 (m, 10H), 3.03 (m, 1H), 2.79 (m, 2H), 2.65-2.56 (m, 3H), 2.42-2.06 (m, 7H), 1.84-1.63 (m, 3H), 1.22 (m, 1H), 1.02 (s, 3H), 0.90 (d, J=7.2 Hz, 3H).

¹⁹F NMR (CDCl₃) (δ, ppm)-166.09 (q).

### General Scheme for Preparation of Conjugates with Linker A

To conjugate eight linker A per antibody, the antibody was buffer exchanged into PBS buffer pH 7.4 at 10 mg/mL concentration, after which 7 equivalents of TCEP was added and incubated at 37° C. for 2 hours. The reduced antibody was then buffer exchanged by PD-10 column (GE Healthcare) with 50 mM borate buffer pH 8.0 containing 2 mM EDTA, after which 12 equivalents of linker A (freshly prepared as 10 mM stock solution in DMSO) was added, the reaction was left at ambient temperature in a tube revolver at 10 rpm for 1 hour. The conjugate containing eight linker-A per antibody was purified using a PD-10 desalting column with PBS buffer pH 7.4. Following elution, the conjugate was further buffer exchanged and concentrated to the desired concentration using Amicon Ultra 15 mL Centrifugal Filters with 30 kDa molecular weight cutoff (MWCO). Mass Spectrometry To determine the Drug to antibody ratio (DAR), the conjugate was incubated with 25 mM of DTT for 30 minutes at 37° C. The reduced conjugate was diluted 50-fold in water and analyzed on a Waters ACQUITY UPLC interfaced to Xevo G2-S QTOF mass spectrometer. Deconvoluted masses were obtained using Waters MassLynx 4.2 Software. Drug to antibody ratios (DAR) were calculated using a weighted average of the peak intensities corresponding to each drug loading species using the formula below:

$$DAR = Σ ( drug load distribution ( \% ) of each Ab with drug load n ) ( n ) / 100$$

### SEC Method

Purity of the conjugate was determined through size exclusion high performance liquid chromatography (SEC-HPLC) using a 20-minute isocratic method with a mobile phase of 0.2M sodium phosphate, 0.2M potassium chloride, 15 w/v isopropanol, pH 6.8. An injection volume of 10 μL was loaded to a TSKgel SuperSW3000 column, at a constant flow rate of 0.35 mL/min. Chromatographs were integrated based on elution time to calculate the purity of monomeric conjugate species.

After synthesis of antibody drug conjugates (ADCs) as described above the naked antibodies and ADCs underwent a quality control process to assess and confirm conjugation, ability to bind to VISTA and endotoxin levels. Also, a control pH-dependent binding anti-VISTA antibody (767-IgG1.3 antibody) which possesses a relatively long in vivo half-life at physiological conditions was synthesized and was analyzed using peptide mapping to confirm its sequence identity and its pH-dependent binding.

### B. Confirmation of Drug Antibody Ratio and Purity by SEC

The conjugation level, presence of high molecular weight (HMW) aggregates, and endotoxin levels were assessed for conjugates following conjugation with linker A (assays performed by Abzena). Briefly, conjugation level was assessed via reverse phase HPLC, mass spectrometry or both. The level of HMW aggregates was assessed via size exclusion column. Endotoxin level was assessed via Charles River endosafe-PTS system, using an LAL test cartridge.

200 μg of the control anti-human VISTA antibody (767-IgG1.3) was digested either with trypsin (1/20 trypsin/protein) at 23° C. for 14h or Lys-C(1/50 Lys-C/protein) at 37° C. for 14h. 80 μg sample was analyzed by mass spectrometry on an Agilent QTOF 6530B. Sequence searches were performed using BioConfirm 9.0.

### C. ELISA Results

### 1. ELISA for Determination of pH Specific Binding

A 96-well flat-bottom plate (Thermo Scientific Nunc Immuno Maxisorp, cat #442404) was coated with 767-IgG1.3 or INX200 diluted to 1 μg/mL in PBS for one hour at room temperature (RT). The wells were washed three times with PT (PBS with 0.05% Tween 20) then blocked with PTB (PBS with 0.05% Tween 20 and 1% BSA) for 1.5 hour at RT.

Biotinylated hIX50 (human VISTA ECD, produced at Aragen Bioscience, biotinylated at ImmuNext) was diluted ranging from 1000 to 0.001 ng/ml in citric acid/phosphate with 0.05% Tween 20 and 1% BSA (CPTB) at pH 6.1, 6.7 or 7.5. The wells were washed three times with citric acid/phosphate with 0.05% Tween 20 (CPT) at pH 6.1, 6.7 or 7.5 then biotinylated hIX50 was added to the wells and incubated for one hour at RT.

After three washes with CPT at pH 6.1, 6.7 or 7.5, streptavidin coupled to HRP (Southern Biotech, cat #7100-05), was used as detection reagent at a dilution of 1/2000 in CPTB at pH 6.1, 6.7 or 7.5 and incubated for one hour at RT. Following three washes with CPT at pH 6.1, 6.7 or 7.5, the ELISA reaction was revealed using TMB (Thermo Scientific, cat #34028) as a colorimetric substrate. After five min at RT, the reaction was stopped with 1M H2S04.

### 2. ELISA for VISTA Binding Confirmation of Naked and Drug-Conjugated Antibodies

A 96-well flat-bottom plates (same as above) were coated with hIX50 (human VISTA ECD, produced at Aragen Bioscience for ImmuNext) at 1 μg/ml in PBS for one hour at RT. After three washes, the wells were blocked with PTB for one hour at RT.

INX200, INX200A, INX201, INX201A, 767-IgG1.3 or 767-IgG1.3A were diluted ranging from 500 to 0.03, 100 to 0.02, or 400 or 0.1 ng/ml in PTB. The wells were washed three times with PT then diluted antibodies were added to the wells and incubated for one hour at RT.

After three washes with PT, mouse anti-human Kappa-HRP (Southern Biotech, cat #9230-05) was used at 1/2000 diluted in PTB as a detection reagent, incubating 1 hour at RT. Following three washes, the ELISA reaction was revealed using TMB substrate. After 5 min at RT, the reaction was stopped with 1M H₂SO₄.

### D. Conjugation Level and SEC Purity Levels for Assessed Antibodies

Conjugation of linker A involved full reduction of interchain disulfides followed by full modification with linker A (as confirmed by mass spectrometry [MS] conjugation assessment). Minimal HMW aggregates were detected as assessed by size exclusion chromatography (SEC) and reported as % purity (See Table 1 below).

TABLE 1
Antibody conjugation level and SEC purity levels (*MS
based conjugation level [orange column in table]
was used for all calculations for dexamethasone equivalence)

| | | | | Puri- | Endotoxin |
|---|---|---|---|---|---|
| | | Conjugation level* | | ty by | level |
| ADC | Lot | MS | RP-HPLC | SEC | (EU/mg) |
| 767-IgG1.3A | JCC-0624-003 | 8.0 | 8.0 | >95% | <0.1 |
| INX200A | JZ-0556-010 | 7.8 | 7.2 | >98% | <0.2 |
| INX200A | JZ-0556-005 | 8.0 | 7.5 | >97% | <0.1 |
| INX201A | JCC-0624-004A | 8 | 7.47 | >95% | <0.1 |
| INX210A | JZ-0556-003 | 8.1 | 7.6 (HIC) | >96% | <0.5 |

### E. Peptide Mapping of 767-IgG1.3

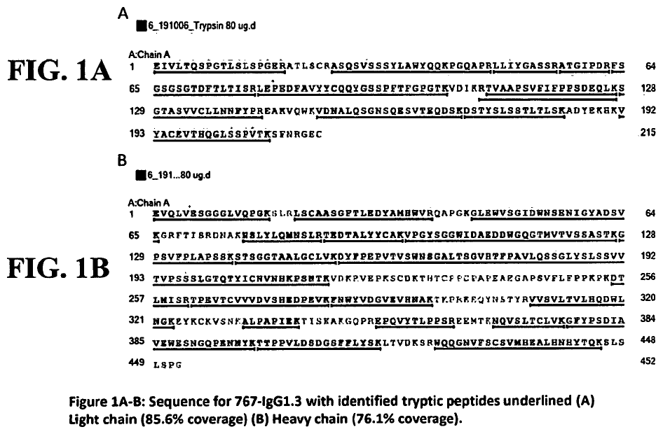

As shown in FIG. 1 trypsin digestion resulted in 85.6% Light chain sequence coverage and 76.1% heavy chain sequence coverage. The Figure shows the sequence for 767-IgG1.3 with identified tryptic peptides underlined (A) Light chain (85.6% coverage) (B) Heavy chain (76.1% coverage).

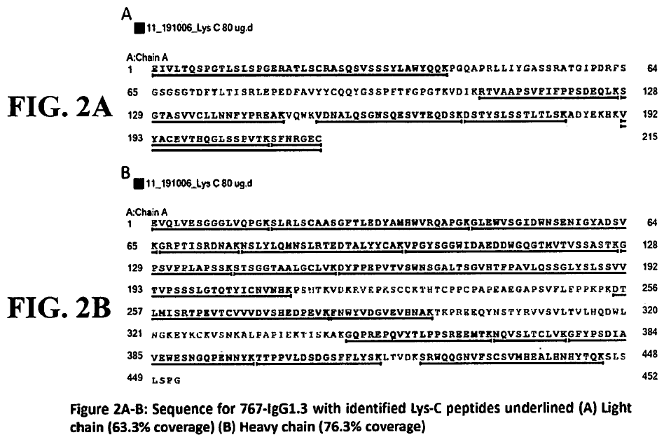

As shown in FIG. 2 Lys-C digestion resulted in 63.3% light chain sequence coverage and 76.3% heavy chain sequence coverage. The Figure shows the sequence for 767-IgG1.3 with identified Lys-C peptides underlined (A) Light chain (63.3% coverage) (B) Heavy chain (76.3% coverage)

Total combined sequence coverage between the trypsin and Lys-C digestion strategies was 91.7% light chain sequence coverage and 80.8% heavy chain sequence coverage. Both light and heavy chains match the intended sequences, as described in WO 2018/169993 A1. Based thereon we confirmed that the cloned and expressed sequence is that of 767-IgG1.3.

### F. Comparison of VISTA Binding of Anti-VISTA Antibodies at Different pH Conditions

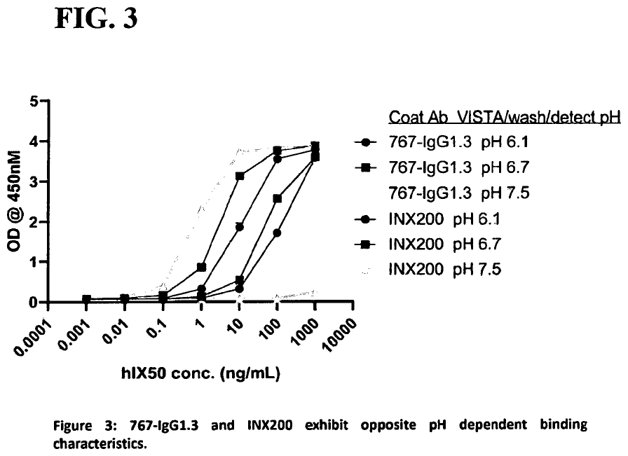

As shown in FIG. 3 plate bound 767-IgG1.3 and INX200 were confirmed to possess opposite anti-VISTA pH dependent binding characteristics. Specifically, FIG. 3 shows that at pH 7.5 (physiological pH), 767-IgG1.3 had minimal binding to soluble VISTA, that binding to VISTA was appreciably higher at pH 6.7, and that the highest degree of binding was at pH 6.1 (lowest pH tested. By contrast, INX200 had the highest degree of binding to soluble VISTA at physiological pH and INX200 binds to soluble VISTA much less as pH decreases (again relative binding at pH 6.7 and pH 6.1 was compared). Therefore, 767-IgG1.3 and INX200 exhibit opposite pH dependent binding characteristics.

### G. Effect of Drug Conjugation on VISTA Binding

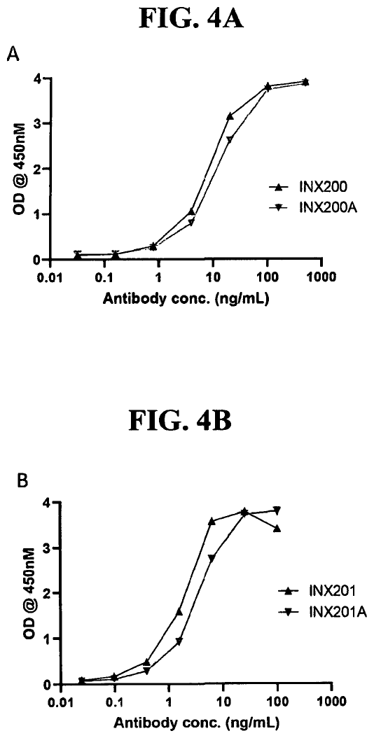

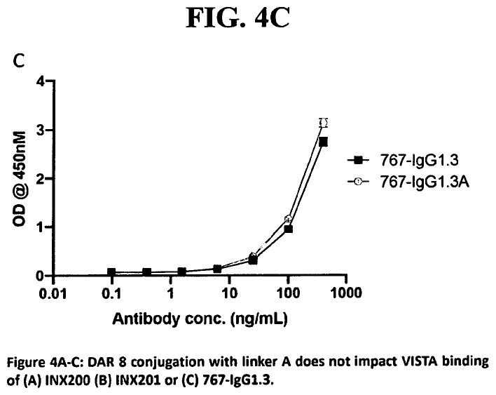

The anti-VISTA antibody drug conjugates identified above were demonstrated in in vitro and in vivo ADC studies to have undergone full reduction of the interchain disulfides with approximately DAR 8 conjugation to dexamethasone-based linker A. Additionally, as shown in FIGS. 4A-C DAR 8 conjugation with linker A was shown to have a negligible effect on the binding of INX200A, INX201A or 767-IgG1.3A to VISTA compared to naked antibodies (FIG. 4A-C).

### H. Conclusions

The above described experiments and data confirm that the control 767-IgG1.3 antibody comprises the same sequence and functional characteristics (pH dependent binding) of the 767-IgG1.3 antibody described previously. These data further confirm that all anti-VISTA antibody drug conjugates which were made underwent full cysteine reduction and that DAR 8 conjugation using a dexamethasone-based linker A resulted in minimal HMW aggregate formation (as assessed by SEC purity) and further showed that such conjugation had negligible impact on the binding of the antibody drug conjugates to human VISTA.

### Example 2: In Vivo Characterization of Exemplary Anti-VISTA Drug Conjugates

### A. ConA Model

Again, because VISTA is highly expressed on most hematopoietic cells, particularly on myeloid cells we selected it as a potential target for anti-inflammatory antibody drug conjugates (ADC's). To assess its potential efficacy in the development of ADCs for potential use in treating autoimmune and inflammatory diseases, the efficacy of Dex-Antibody drug conjugates was evaluated in a short-term model of concanavalin A-induced liver inflammation (ConA-induced hepatitis).

This model involves the intravenous (i.v.) injection of the plant lectin concanavalin A (ConA) in mice and comprises a widely used model for acute immune mediated hepatitis in mice. In contrast to several other models for acute hepatic damage, ConA-induced injury is primarily driven by the activation and recruitment of T cells to the liver. Hence, the ConA model has unique features with respect to its pathogenesis and important similarities to immune-mediated hepatitis in humans, such as autoimmune hepatitis, acute viral hepatitis or distinct entities of drug toxicity leading to immune activation. The ConA model is characterized by a burst of pro-inflammatory cytokines that can be monitored as early as 6h post injection. By 24h, high levels of pro-inflammatory cytokines are still detected, and liver damage/necrosis can be observed by histopathology. We took advantage of this model by mainly monitoring cytokine response at 6h post ConA injection. As discussed below and shown in the Figures these studies showed that Dex treatment has a dose dependent effect on G-CSF, IFNγ, IL-2, IL-6, IL-12p40, IL-12p70 and KC so our studies focused on measuring some of these cytokines.

### B. Study Design

In these experiments, mice received antibody or Dex treatments ˜15 h before disease initiation. Concanavalin A dosing was adjusted to generate acute but non-lethal inflammation at 6 hr, established in preliminary experiments. Blood was collected at 6h post ConA i.v. injection, and plasma isolated for cytokine analyses.

The objective of the in vivo studies was to evaluate the relative efficacy of these INX human VISTA antibodies conjugated to dexamethasone via an esterase sensitive linker as compared to free Dex in ConA-induced hepatitis. Particularly, in vivo studies were conducted to evaluate the efficacy of anti-human VISTA antibodies (INX210 [silent IgG2 Fc], INX200 [silent IgG1 Fc] and 767.3-IgG1.3 [control pH sensitive antibody]) naked or conjugated to Dexamethasone in the Concanavalin A-induced hepatitis model (respectively EXPERIMENT 1, 2, and 3).

These experiments were conducted in human VISTA knock-in (hVISTA KI) mice. hVISTA KI mice have the human VISTA cDNA knocked-in in place of the mouse VISTA gene, and express human VISTA both at RNA and protein levels. Also, in order to rule out gender-based differences inefficacy these experiments were performed in female and male mice. All animals received treatment (antibody or dexamethasone) 15h before Concanavalin A (ConA) injection. Mice were then bled at 6h post ConA injection and cytokine responses evaluated as markers of disease progression.

### C. Methods and Materials

### Anti-VISTA Antibodies and Conjugates

INX200: Humanized anti-human VISTA antibody on a human IgG1/kappa backbone with L234A/L235A silencing mutations in the Fc region which possesses a very short serum half-life at physiological pH (see Table 7 infra) and comprising the variable heavy and light sequences and IgG1 Fc region contained in FIG. 8).

INX200A: INX200 conjugated to dexamethasone drug via the interchain disulfides with a drug/antibody ratio (DAR) of ˜8. The linker/payload (A) consists of an esterase sensitive linker with a dexamethasone payload (as described in Graverson et al, 2012).

INX201: Humanized anti-human VISTA antibody on a human IgG1/kappa backbone with L234A/L235A/E269R/K322A silencing mutations in the Fc region which possesses a very short serum half-life at physiological pH (see Table 7 infra) having variable heavy and light sequences and IgG1 Fc region contained in FIG. 8).

INX201A: INX201 antibody conjugated to dexamethasone drug via the interchain disulfides with a drug/antibody ratio (DAR) of 8. The linker/payload (A) again consists of an esterase sensitive linker with a dexamethasone payload (as described in Graverson et al, 2012).

INX210: Humanized anti-human VISTA antibody on a human IgG2/kappa backbone with V234A/G237A/P238S/H268A/V309L/A330S/P331S silencing mutations in the Fc region having variable heavy and light sequences and IgG1 Fc region contained in FIG. 8) which possesses a very short serum half-life (see Table 7 infra) (Vafa et al, 2014).

INX210A: INX210 antibody conjugated to drug via the interchain disulfides with a drug/antibody ratio (DAR) of ˜8. The linker/payload (A) again consists of an esterase sensitive linker with a dexamethasone payload (as described in Graverson et al, 2012).

767-IgG1: Control humanized anti-human VISTA antibody developed by Five Prime Therapeutics and Bristol-Myers Squibb Company on a human IgG1/kappa backbone with L234A/L235E/G237A silencing mutations in the Fc region having variable heavy and light sequences and IgG1 Fc region contained in FIG. 8) which possesses a much longer serum half-life at physiological pH (more than 24 hours in rodents and primates)). This antibody was designed to bind VISTA at low pH (e.g. pH 6) but to have minimal binding at physiological pH (pH 7.4) (WO 2018/169993 A1).

767-IgG1A: 767-IgG1 antibody conjugated to drug via the interchain disulfides with a drug/antibody ratio (DAR) of ˜8. The linker/payload (A) again consists of an esterase sensitive linker with a dexamethasone payload (as described in Graverson et al, 2012).

### Antibody Dosing:

All antibodies were diluted in PBS and injected intraperitoneal (i.p.) in a volume of 0.2 ml to deliver a dose of 10 mg/Kg.

### Dexamethasone

Dexamethasone (sterile injection from Phoenix, NDC 57319-519-05), was diluted in PBS and dosed at 5, 2, 0.2 and 0.02 mg/Kg via i.p. injection.

### Concanavalin A

Concanavalin A was obtained from Sigma Aldrich (C2010). Depending on its lot, ConA can be more or less virulent so preliminary experiments were always conducted to define the best ConA dosing to generate acute but non-lethal inflammation at 6 hr: 15 mg/Kg for EXPERIMENT 1 and 2 (lot #SLBX7517) and 7.5 mg/Kg for Experiment 3 and 4 (lot #SLCC2664).

### Mice

hVISTA mice were bred at Sage Labs (Boyertown, PA). The mice, aged 8-12 weeks, first transited for 3 weeks in our quarantine facility, and then were transferred to the regular facility. They were acclimated for 1 to 2 weeks prior to experiment initiation.

### Blood Draw and Preparation

Peripheral blood was harvested from the retro-orbital cavity using a glass Pasteur pipette that was 1st rinsed with heparin to prevent coagulation. Blood was then centrifuged at 400 rcf for 5 min and plasma collected and stored at −80° C. before cytokine analysis.

### Plasma Cytokine Analysis

Cytokine analyses were conducted on 25 μl of plasma using a Millipore mouse 7-plex platform.

### Experiment 1 and 2: Cytokines Included in the Analysis for In Vivo Studies were G-CSF, IL-2 IFNγ, IL-6, IL-12p40, IL-12p70 and KC

Experiment 3: For in vivo Experiment 3, only G-CSF and KC were analyzed via ELISA using R&D Duo sets for G-CSF (DY414-05; Expected <100,000 pg/mL of G-CSF and likely <50,000 pg/mL-Kit detection level: 2000 pg/mL-31.3 pg/mL) and KC (DY453-05; expected <120,000 pg/mL and likely <50,000 pg/mL-Kit detection level: 1000 pg/mL-15.6 pg/mL).

### D. Results

### Experiment 1: INX210A Efficacy in ConA-Induced Hepatitis in Female hVISTA KI Mice

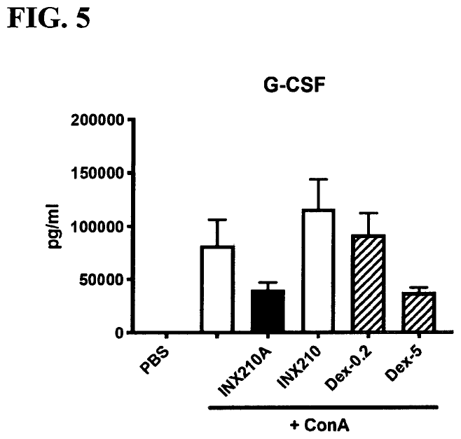

FIG. 5 shows G-CSF changes at 6h post ConA in peripheral blood of female hVISTA KI mice. Plasma concentrations measured using a mouse 7-plex (SEM; n=5/group) (Dosing: Dex-0.2=0.2 mg/Kg, Dex-2=2 mg/Kg, INX210 and INX210A at 10 mg/Kg, [INX210A provided 0.2 mg/kg dex payload]).

As shown in the Figure, treatment with INX210A showed some efficacy (though non-significant) in controlling ConA-induced G-CSF upregulation, comparable to Dex treatment at 5 mg/Kg. By contrast, the non-Dex conjugated antibody INX210 or Dex administered 0.2 mg/Kg (which is the molar equivalent of Dex delivered by INX210A) had no anti-inflammatory impact.

Because we observed high levels of intragroup variability in the ConA response, the data from the 6 other cytokines is not included as it varied too much for interpretation. This is not unexpected because when experiments are run in female mice the effect of ConA is highly dependent on the hormonal state of the animal. While female mice may show a higher susceptibility to ConA, they also show greater variation in the disease outcome. All subsequent ConA experiments were run in male mice.

### Experiment 2: INX210A Efficacy in ConA-Induced Hepatitis in Male hVISTA KI Mice

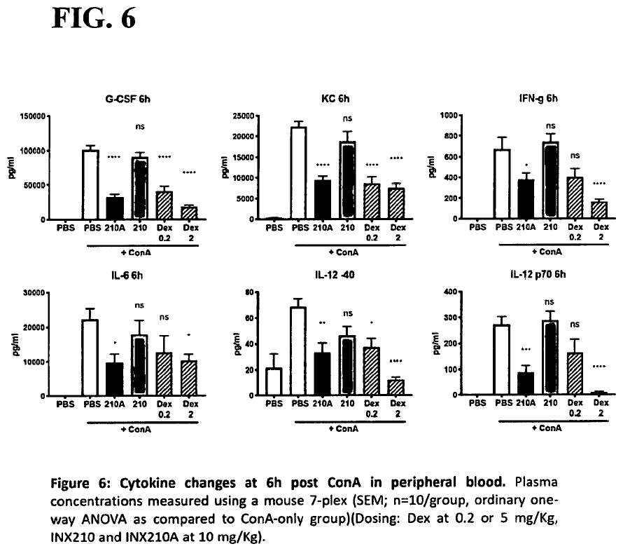

FIG. 6 shows cytokine changes at 6h post ConA in peripheral blood in male hVISTA KI mice. Plasma concentrations measured using a mouse 7-plex (SEM; n=10/group, ordinary one-way ANOVA as compared to ConA-only group) (Dosing: Dex at 0.2 or 5 mg/Kg, INX210 and INX210A at 10 mg/Kg).

As has been previously reported in the literature, male mice displayed more consistent cytokine responses to ConA. Six out of 7 cytokines analyzed showed significant reduction (1 to 3 fold) when compared to the untreated ConA group at 6h following INX210A treatment (FIG. 6) The decreases were intermediate between Dex 0.2 mg/kg (the molar equivalent of INX210A dex payload) and Dex 5 mg/kg. By contrast no efficacy was noted in the INX210 treated group.

### Experiment 3: Dose Response for INX200A on Concanavalin A-Induced Hepatitis in DDE1 Male Mice

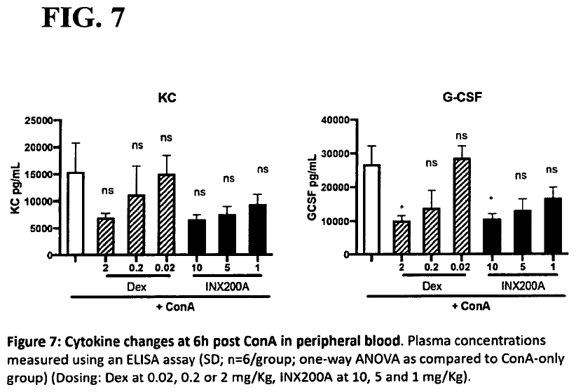

FIG. 7 shows cytokine changes at 6h post ConA in peripheral blood in DDE1 male mice. In the experiments cytokine plasma concentrations measured using an ELISA assay (SD; n=6/group; one-way ANOVA as compared to ConA-only group) (Dosing: Dex at 0.02, 0.2 or 2 mg/Kg, INX200A at 10, 5 and 1 mg/Kg).

To evaluate if the ADC INX200A confers an efficacy boost, the response to various Dex dosages to the equivalent Dex payload from ADC (0.2 mg/Kg of free Dex=INX200A at 10 mg/Kg; 0.02 mg/Kg of free Dex=INX200A at 1 mg/Kg) were compared. As can be seen from the data in FIG. 7, while free Dex has lost efficacy in controlling cytokine response at 0.02 mg/Kg, INX200A at 1 mg/Kg is still efficacious. More generally the data show the following:

### Experiment 1

In female hVISTA KI mice, INX210 when conjugated to Dex (INX210A), showed efficacy in controlling ConA-induced G-CSF responses.

### Experiment 2

Male hVISTA KI mice display more consistent responses to ConA injury.
INX210 when conjugated to Dex (INX210A) showed efficacy in controlling ConA-induced cytokine responses. The naked antibody had no efficacy.
INX210A dosed at 10 mg/Kg delivers ˜0.2 mg/Kg of Dex; Efficacy observed with
INX210A was comparable to efficacy of free Dex at 0.2 mg/Kg.

### Experiment 3

The dose response experiment showed improved/boost in efficacy when Dex payload is delivered via the ADC INX200A: while free Dex at 0.02 mg/Kg has no efficacy, the molar equivalent delivered via ADC shows high potency.

### Conclusions

We show that the anti-VISTA antibody (INX210) when conjugated to Dex (INX210A) can prevent ConA induced inflammation as efficiently or better than free Dex at equivalent molar dosage of Dex. Unconjugated, INX210, has no impact. We also show that conjugating Dex to the anti-VISTA antibody INX200 improved Dex delivery as we show that free Dex at 0.02 mg/Kg has no efficacy while the molar equivalent delivered via ADC has high potency.

### Example 3: Synthesis of Exemplary Steroid Payloads and Antibody Drug Conjugates

### Procedures for Synthesis of Steroid Payloads and Conjugates

In this example we describe the synthesis of novel steroids according to the invention, conjugates wherein said steroid is coupled to a linker and/or a bifunctional or trifunctional group which permits attachment of the steroid linker conjugate to an antibody, and antibody drug conjugates (ADCs) comprising said steroid coupled to a linker and/or a bifunctional or trifunctional group coupled to an antibody, optionally an anti-VISTA antibody that binds to human VISTA at physiologic pH and which comprises a short pK, or an antibody or antibody fragment which targets another antigen selectively expressed on immune cells, typically human immune cells.

As noted previously these steroids in general will comprise a glucocorticoid agonist compounds and possess the structure of Formula I, II or III previously disclosed. Exemplary compounds of Formula I, II and III are depicted in FIG. 118A-O. The synthesis of these and other exemplary compounds is described herein.

### General Procedures

The following general procedures were used for liquid chromatography (preparative or analytical) and nuclear magnetic resonance.

### Liquid Chromatography

Unless noted otherwise, the following conditions were used for high pressure liquid chromatography (HPLC) purification or for liquid chromatography-mass spectrometry (LC-MS):

### LCMS Method A

Sample analysis according to this method was performed on an Agilent 1260 LCMS-4-QUAD system with an Onyx™ Monolithic C18 LC Column, 50×2 mm. Samples were run using a gradient of 5-95% A in B over 6 minutes, where A=0.05% AcOH in water/ACN (95:5 v/v) and B=0.05% AcOH in ACN.

### LCMS Method B

Sample analysis according to this method was performed on a Waters Acquity LCMS-5-SQD system with a Kinetex® 1.7 μm C18 100 Å, LC Column 50×2.1 mm. Samples were run using a gradient of 10-95% A in B over 2.5 minutes, where A=0.02% formic acid in water and B=0.05% formic acid in ACN.

### Following are the LCMS Methods Used for the Analysis of Final Targets:

### LCMS Method-1:

Column Details: X-BRIDGE BEH 2.1*50 mm 2.5 μm

Machine Details:—Water Acquity UPLC-H Class equipped with PDA and Acquity SQ detector, Column temperature: 35° C., Auto sampler temperature: 5° C., Mobile Phase A: 0.1% Formic acid in Milli Q water (pH=2.70), Mobile Phase B: 0.1% Formic acid in Milli Q water: Acetonitrile (10:90).

Mobile phase gradient details: T=0 min (97% A, 3% B) flow: 0.8 mL/min; T=0.75 min (97% A, 3% B) flow: 0.8 mL/min; gradient to T=2.7 min (2% A, 98% B) flow: 0.8 mL/min; gradient to T=3 min (0% A, 100% B) flow: 1 mL/min; T=3.5 min (0% A, 100% B) flow: 1 mL/min; gradient to T=3.51 min (97% A, 3% B) flow: 0.8 mL/min; end of run at T=4 min (97% A, 3% B), Flow rate: 0.8 mL/min, Flow rate: 0.8 mL/min, Run Time: 4 min, UV Detection Method: PDA.

### Mass Parameter:

Probe: ESI, Mode of Ionization: positive and negative, Cone voltage: 30V and 10 V, capillary voltage: 3.0 KV, Extractor Voltage: 1V, Rf Lens: 0.1 V, Temperature of source: 120° C., Temperature of Desolvation: 400° C. Cone Gas Flow: 100 L/hour, Desolvation Gas flow: 800 L/hour.

### LCMS Method-2:

Column Details: Xtimate C18 4.6*150 mm 5 μm

Machine Details: Waters 996 Photodiode Array Detector equipped with Waters Micro mass ZQ detector, Column temperature: 35° C., Auto sampler temperature: 15° C., Mobile Phase A: 5 mM Ammonium Acetate and 0.1% Formic acid (pH=3.50) in Milli Q water, Mobile Phase B: Methanol

Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.0 mL/min, Run Time: 17 min, UV Detection Method: PDA.

### Mass Parameter:

Probe: ESI, Mode of Ionization: Positive and Negative, Cone voltage: 30 and 10V, capillary voltage: 3.0 KV, Extractor Voltage: 2V, Rf Lens: 0.1V, Temperature of source: 120° C., Temperature of Probe: 400° C., Cone Gas Flow: 100 L/Hr, Desolvation Gas flow: 800 L/Hr.

### LCMS Method-3:

Column Details: Sunfire C18 150×4.6 mm, 3.5 μm

Machine Details: Agilent 1260 Infinity-II and G6125C (LC/MSD) mass detector, Column temperature: 35° C., Auto sampler temperature: 15° C., Mobile Phase A: 5 mM Ammonium Acetate and 0.1% Formic acid (pH=3.50) in Milli Q water, Mobile Phase B: Methanol Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.0 mL/min, Run Time: 17 min, UV Detection Method: PDA.

### Mass Parameter:

Probe: MMI, Mode of Ionization: (ESI) positive and negative, Fragment voltage: 30V & 70 V, capillary voltage: 3000 V, Gas temperature of source: 325° C., Temperature of vaporizer: 225° C., Gas Flow: 12 L/min, Nebulizer: 50.

### HPLC Method-1:

Column Details: Sunfire C18 (150 mm×4.6 mm), 3.5 μm

Machine Details: Agilent Technologies. 1260 Series, Infinity-II with PDA detector, Column temperature: 35° C., Auto sampler temperature: 15° C., Mobile Phase A: 0.05% Trifluoroacetic acid in Milli Q water (pH=2.2), Mobile Phase B: Acetonitrile.

Mobile phase gradient details: T=0 min (90% A, 10% B) flow: 1.0 mL/min; T=7.0 min (10% A, 90% B) flow: 1.0 mL/min; gradient to T=9.0 min (00% A, 100% B) flow: 1.0 mL/min; gradient to T=14 min (00% A, 100% B) flow: 1.0 mL/min; T=14.01 min (90% A, 10% B) flow: 1 mL/min; end of run at T=17 min (90% A, 10% B) flow: 1.0 mL/min, Flow rate: 1.0 mL/min, Run Time: 17 min, UV Detection Method: PDA.

### HPLC Method-2

Column Details: Atlantis C18 (150 mm×4.6 mm),5.0 μm or Welch C18 (150 mm×4.6 mm),5.0 μm

Machine Details:—Waters Alliance e2695 with 2998 PDA detector, Column temperature: 35° C., Auto sampler temperature: 15° C., Mobile Phase A: 0.1% Ammonia in Milli Q water (pH=10.5), Mobile Phase B: Acetonitrile.

Mobile phase gradient details: T=0 min (90% A, 10% B) flow: 1.0 mL/min; T=7.0 min (10% A, 90% B) flow: 1.0 mL/min; gradient to T=9.0 min (00% A, 100% B) flow: 1.0 mL/min; gradient to T=14 min (00% A, 100% B) flow: 1.0 mL/min; T=14.01 min (90% A, 10% B) flow: 1 mL/min; end of run at T=17 min (90% A, 10% B) flow: 1.0 mL/min, Flow rate: 1.0 mL/min, Run Time: 17 min, UV Detection Method: PDA.

HPLC details: Waters Alliance e2695 with 2998 PDA detector; Column Details: Atlantis C18 (150 mm×4.6 mm),5.0 μm or Welch C18 (150 mm×4.6 mm), 5.0 μm; Mobile Phase A: 0.1% Ammonia in Milli Q water (pH=10.5), Mobile Phase B: Acetonitrile; Flow rate: 1.0 mL/min, Run Time: 17 min

### NMR

The following conditions were used for obtaining proton nuclear magnetic resonance (NMR) spectra: NMR spectra were recorded on an ¹H NMR (400 MHZ) Bruker Advancer-III HD FT-NMR spectrophotometer (Bruker, USA). The crude NMR data was analyzed using Topspin 3.6.3 software.

Chemical shifts are reported in parts per million (ppm) downfield from the position of TMS inferred by the deuterated NMR solvent. Apparent multiplicities are reported as: singlet-s, doublet-d, triplet-t, quartet-q, or multiplet-m. Peaks that exhibit broadening are further denoted as br. Integrations are approximate. It should be mentioned that integration intensities, peak shapes, chemical shifts and coupling constants can be dependent on solvent, concentration, temperature, pH and other factors.

### Experimental Details

All reactions were conducted under a dry nitrogen atmosphere unless otherwise stated. All the key chemicals were used as received. All other commercially available materials, such as solvents, reagents and catalyst were used without further purification. Reactions were monitored by thin layer chromatography (TLC) using pre-coated Merck silica gel 60F254 aluminium sheets (Merck, Germany). The visualization of TLC plates was accomplished using UV light, ninhydrin spray, and iodine vapors. Column chromatographic separations were carried out using 230-400 mesh, 100-200 mesh and 60-120 mesh silica gel or C18 silica as stationary phase using appropriate mobile phase.

### Synthesis of S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid (INX J)

### Reaction Scheme

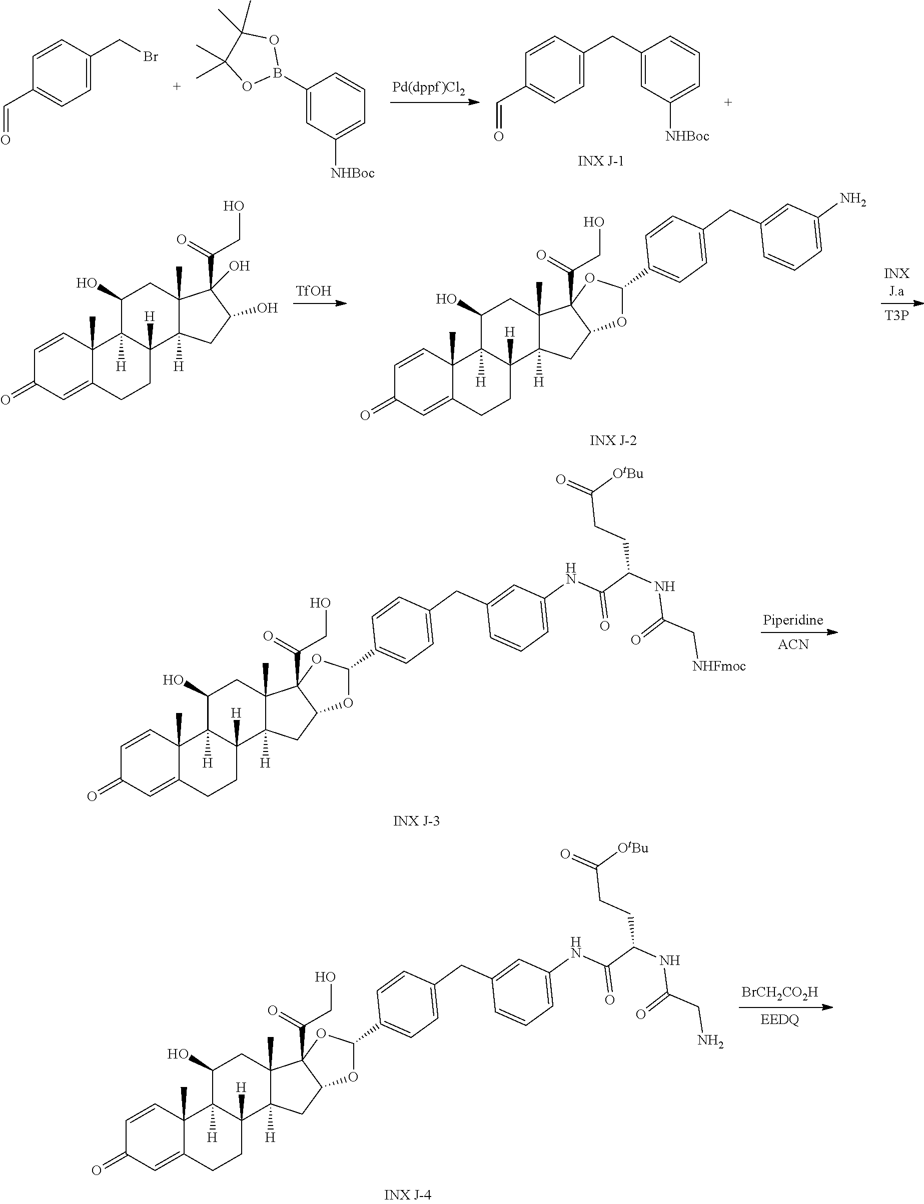

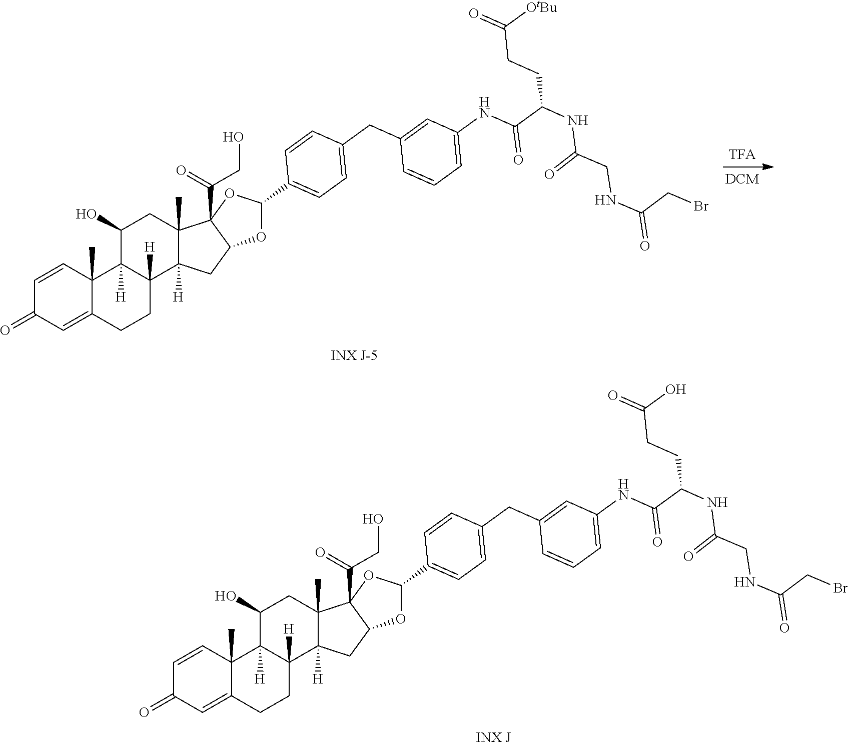

### Synthesis of (S)-2-(2-((((9H-fluoren-9-yl)methoxy)carbonyl)amino)acetamido)-5-(tert-butoxy)-5-oxopentanoic acid (INX J.a)

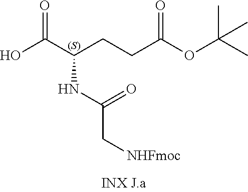

### Procedure

A round bottom flask was charged with Fmoc-Gly-OSu (1.0 g, 2.535 mmol, 1.0 eq), H-Glu(OtBu)-OH (0.6183 g, 3.043 mmol, 1.2 eq), and sodium bicarbonate (0.4260 g, 5.07 mmol, 2.0 eq). A solution of water and 1,4-dioxane (1:1, 26 mL) was added and the mixture was allowed to stir overnight at room temperature. Starting material consumption was confirmed by LCMS and the solvent was reduced, removing the dioxane but leaving the water. The mixture was then acidified to pH 2-3, added to a separatory funnel, and extracted with 5:1 isopropyl acetate/isopropanol (3×100 mL). Combined organics were dried over Na₂SO₄, filtered, reduced, loaded onto an Isco C18 Aq 100 g reverse phase column, and eluted with a mobile phase of 0-100% acetonitrile (0.05% AcOH additive) in H₂O (0.05% AcOH additive). The fractions containing pure product were combined, frozen, and lyophilized to afford 0.9982 g of INX J.a, 82% yield, as a white solid. LCMS Method B (ESI+): C₂₆H₃₁N₂O₇ [M+H]⁺ requires 483.21, found 483.25 at 1.14 minutes.

### Synthesis of tert-butyl (3-(4-formylbenzyl)phenyl)carbamate (INX J-1)

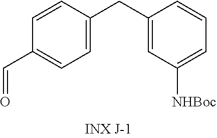 [SMILES: CC(C)(C)OC(=O)Nc1cccc(Cc2ccc(C=O)cc2)c1]

### Procedure

A round bottom flask was back-filled with argon and charged with tert-butyl (3-(4,4,5,5-tetramethyl-1,3,2-dioxaborolan-2-yl)phenyl)carbamate (4.2765 g, 13.40 mmol, 1.0 eq), 4-bromomethylbenzaldehyde (4.0 g, 20.1 mmol, 1.5 eq), potassium carbonate (9.2594 g, 67.0 mmol, 5.0 eq), and [1,1′-Bis(diphenylphosphino)ferrocene]dichloropalladium-dichloromethane complex (0.3841 g, 0.469 mmol, 0.035 eq). Anhydrous THF (84 mL) was added to the flask, which was then equipped with a reflux condenser and heated to 80° C. for 16 h. Starting material consumption was confirmed by LCMS and the mixture was then cooled, diluted with water (200 mL), added to a separatory funnel, and extracted with EtOAc (3×100 mL). The combined organic extracts were dried over Na₂SO₄, filtered, reduced, and loaded onto an Isco Rf Gold 80 g SiO₂ column and eluted with a mobile phase of 0-100% EtOAc in hexanes. The fractions containing pure product were combined and reduced to afford 3.529 g of compound INX J-1, 85% yield, as a clear oil which crystallized overnight after removal from reduced pressure. LCMS Method A (ESI−): C₁₉H₂₀NO₃ [M−H]⁻ requires 310.15, found 310.1 at 3.080 minutes.

### Synthesis of (6aR,6bS,7S,8aS,8bS,10R,11aR,12aS,12bS)-10-(4-(3-aminobenzyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX J-2)

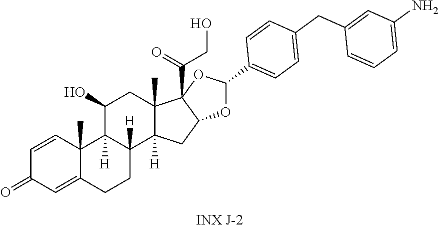

### Procedure

A round bottom flask was charged with 16-α-hydroxyprednisolone (3.30 g, 8.765 mmol, 1.0 eq), aldehyde INX J-1 (3.0023 g, 9.641 mmol, 1.1 eq), and MgSO₄ (3.1659 g, 26.29 mmol, 3.0 eq). The solids were suspended in acetonitrile (88 mL) and the mixture was cooled to 0° C., whereupon trifluoromethanesulfonic acid (3.9 mL, 43.83 mmol, 5.0 eq) was added dropwise. After 10-20 minutes the reaction turned pink, and the starting material was fully consumed after 1 h. The solvent was reduced and the crude was purified in two batches, each being loaded onto to an Isco C18 Aq 275 g reverse phase column and eluted with a mobile phase of 5-100% acetonitrile (0.05% AcOH additive) in H₂O (0.05% AcOH additive). The fractions from both batches containing pure product were combined, frozen, and lyophilized to afford 2.50 g of INX J-2, 50% yield, as a white solid. LCMS Method A (ESI+): C35H40NO6 [M+H]⁺ requires 570.28, found 570.3 at 2.572 minutes.

### Synthesis of tert-butyl(S)-4-(2-((((9H-fluoren-9-yl)methoxy)carbonyl)amino)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (INX J-3)

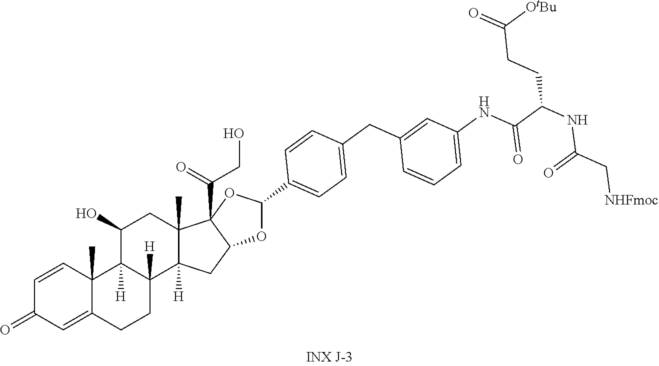

### Procedure

DMF (2.3 mL) was added to a round bottom flask that was charged with bis-amino acid INX J.a (0.3074 g, 0.6372 mmol, 1.1 eq). Aniline INX J-2 (0.330 g, 0.579 mmol, 1.0 eq) was then added, followed by triethylamine (0.24 mL, 1.73 mmol, 3.0 eq). The solution was cooled to 0° C., whereupon a solution of 50% propanephosphonic acid anhydride in DMF (0.70 mL, 1.1586 mmol, 2.0 eq) was added. The mixture was allowed to stir 16 h while warming to room temperature. Once reaction completion was confirmed by LCMS, the crude mixture was directly loaded onto an Isco C18 Aq 50 g reverse phase column and eluted with a mobile phase of 0-100% acetonitrile (0.05% AcOH additive) in H₂O (0.05% AcOH additive). The fractions containing pure product were combined, frozen, and lyophilized to afford 0.200 g of INX J-3, 33% yield, as a white solid. LCMS Method A (ESI+): C61H68N3012 [M+H]⁺ requires 1034.47, found 1034.4 at 3.073 minutes.

### Synthesis of tert-butyl(S)-4-(2-aminoacetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate AcOH salt (INX J-4)

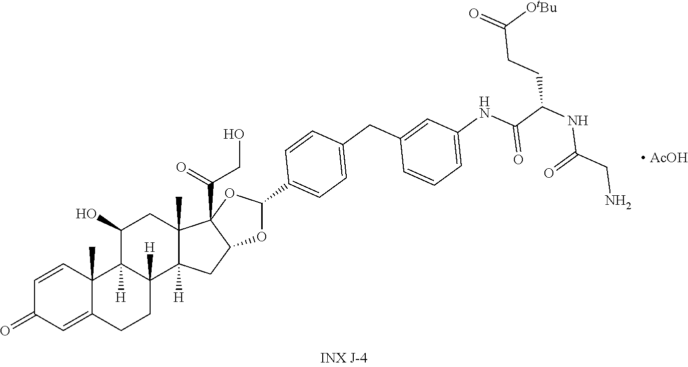

### Procedure

A vial was charged with compound INX J-3 (0.080 g, 0.0774 mmol, 1.0 eq) which was then dissolved in acetonitrile (0.50 mL) and piperidine (62 μL). The mixture was allowed to stir until all starting material was deprotected, 30 min. The solvent was reduced, the crude was diluted in DMSO, and loaded onto an Isco C18 Aq 15.5 g reverse phase column and eluted with a mobile phase of 0-100% acetonitrile (0.05% AcOH additive) in H₂O (0.05% AcOH additive). The fractions containing pure product were combined, frozen, and lyophilized to afford 0.0423 g of INX J-4·AcOH, 63% yield, as a clear oil. LCMS Method A (ESI+): C₄₆H₅₈N₃O₁₀ [M+H]⁺ requires 812.40, found 812.4 at 2.638 minutes.

### Synthesis of tert-butyl(S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (INX J-5)

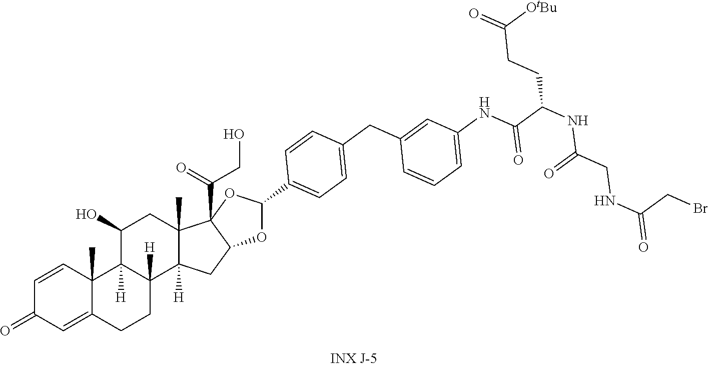

### Procedure

A vial was charged with 2-bromoacetic acid (0.0092 g, 0.0665 mmol, 2.1 eq) and DMF (0.33 mL). N-Ethoxycarbonyl-2-ethoxy-1,2-dihydroquinoline (0.0156 g, 0.0632 mmol, 2.0 eq) was added and the mixture was allowed to stir for ˜90 minutes. Amine INX J-4·AcOH (0.0270 g, 0.0309 mmol, 1.0 eq) was then added to the solution along with sodium bicarbonate (0.0140 g, 0.1665 mmol, 5.4 eq) and the mixture was allowed to stir for 2 h (until all INX J-4 was consumed). Once reaction completion was confirmed by LCMS, the crude mixture was directly loaded onto an Isco C18 Aq 5.5 g reverse phase column and eluted with a mobile phase of 0-100% acetonitrile (0.05% AcOH additive) in H₂O (0.05% AcOH additive). The fractions containing pure product were combined, frozen, and lyophilized to afford 0.0100 g of INX J-5, 35% yield, as a white solid. LCMS Method A (ESI+): C₄₈H₅₉BrN₃O₁₁ [M+H]⁺ requires 932.33, found 932.2 at 2.926 minutes.

### Synthesis of (S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid (INX J)

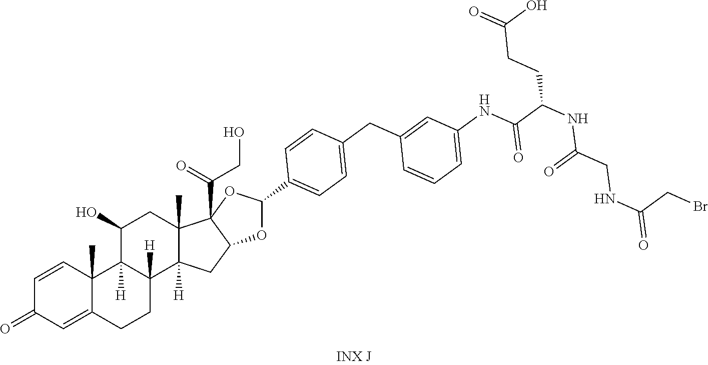

A vial was charged with tert-butyl ester INX J-5 (0.010 g, 0.01072 mmol, 1.0 eq), which was dissolved in a solution of 50% TFA in DCM (0.200 mL) and allowed stir for 1 h. Once reaction completion was confirmed by LCMS, the solvent was removed, the residue was dissolved in DMSO, and loaded onto an Isco C18 Aq 5.5 g reverse phase column and eluted with a mobile phase of 0-100% acetonitrile (0.05% AcOH additive) in H₂O (0.05% AcOH additive). The fractions containing pure product were combined, frozen, and lyophilized to afford 0.0033 g of INX J, 35% yield, as a white solid. LCMS Method A (ESI+): C₄₄H₅₁BrN₃O₁₁ [M+H]⁺ requires 876.26, found 877.2 at 2.524 minutes.

### Synthesis of S)-4-(2-(2-bromoacetamido)acetamido)-5-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid (INX L)

### Reaction Scheme

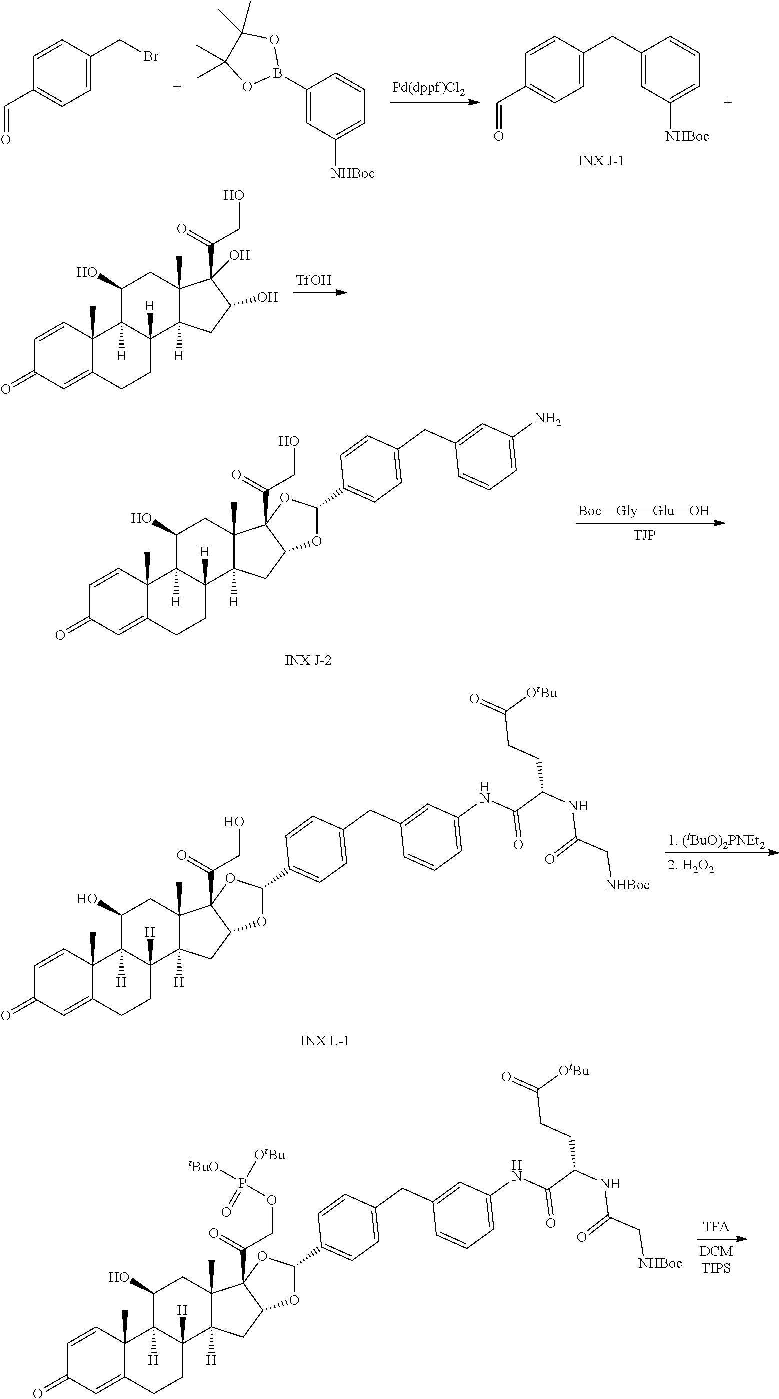

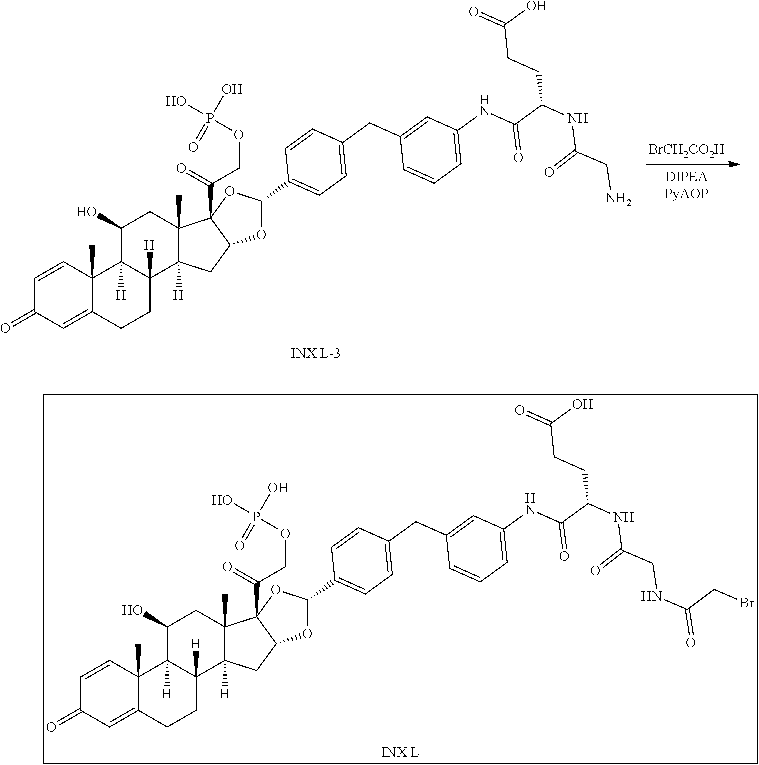

### Synthesis of (S)-5-(tert-butoxy)-2-(2-((tert-butoxycarbonyl)amino)acetamido)-5-oxopentanoic acid (Boc-Gly-Glu(OtBu)-OH)

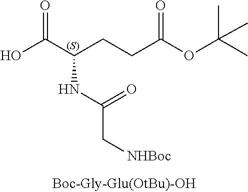

A round bottom flask was charged with Boc-Gly-OSu (12.0 g, 44.07 mmol, 1.0 eq), H-Glu(OtBu)-OH (9.8524 g, 48.47 mmol, 1.1 eq), and sodium bicarbonate (7.4040 g, 88.14 mmol, 2.0 eq). A solution of water and 1,4-dioxane (1:1, 220 mL) was added and the mixture was allowed to stir overnight at room temperature. Starting material consumption was confirmed by LCMS and the solvent was reduced, removing the dioxane but leaving the water. The mixture was then acidified to pH 2-3, forming a precipitate which was then filtered and dried on a lyophilizer to afford 14.0152 g of Boc-Gly-Glu(OtBu)-OH, 88% yield, as a white solid. LCMS Method A (ESI+): C₁₆H₂₉N₂O₇ [M+H]⁺ requires 361.19, found 361.2 at 2.122 minutes.

### Synthesis of tert-butyl(S)-4-(2-((tert-butoxycarbonyl)amino)acetamido)-5-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (INX L-1)

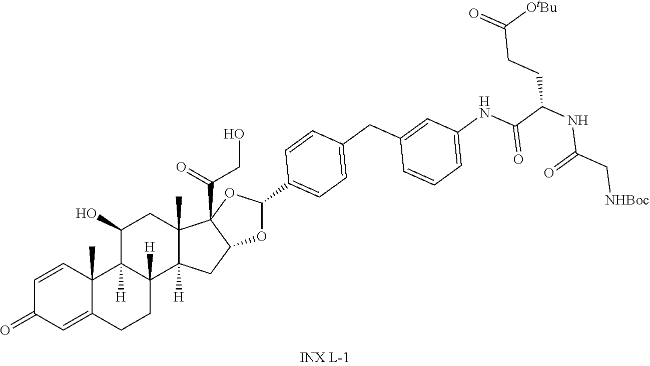

### Procedure

An oven-dried vial under inert atmosphere was charged with amine INX J-2 (0.8200 g, 2.63 mmol, 1.0 eq), Boc-Gly-Glu(OtBu)-OH (2.5916 g, 7.197 mmol, 2.73 eq), ((7-Azabenzotriazol-1-yloxy)tripyrrolidinophosphonium hexafluorophosphate) (2.2515 g, 4.318 mmol, 1.64 eq), and DMF (15 mL mL). Next, N,N-Diisopropylethylamine (1.5 mL, 8.636 mmol, 3.3 eq) was added and the mixture was allowed to stir until all the amine was consumed, 1 h. The crude solution was then added directly to an Isco C18 Aq 100 g reverse phase column and eluted with a mobile phase of 0-100% acetonitrile (0.05% TFA additive) in H₂O (0.05% TFA additive). The fractions containing pure product were combined, frozen, and lyophilized to afford 0.4404 g of INX L-1, 34% yield, as a white solid. LCMS Method A (ESI+): C₅₁H₆₆N₃O₁₂ [M+H]⁺ requires 912.46, found 912.4 at 2.524 minutes.

### Synthesis of tert-butyl(S)-4-(2-((tert-butoxycarbonyl)amino)acetamido)-5-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (INX L-2)

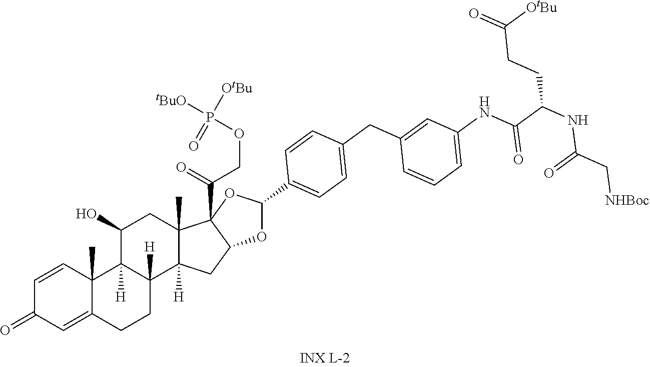

### Procedure

An oven-dried vial under inert atmosphere was charged with tert-butyl ester INX L-1 (0.200 g, 0.220 mmol, 1.0 eq) and DMF (0.50 mL). Next, 1-H tetrazole (0.1540 g, 2.20 mmol, 10 eq) and di-tert-butyl N,N-diethylphosphoramidite (1.311 g, 5.265 mmol, 24.0 eq) were added and the mixture was allowed to stir for 72 h to achieve 90% conversion. Hydrogen peroxide (2 mL) was added and the mixture was allowed to stir for 1 h before being loaded onto an Isco C18 Aq 50 g reverse phase column and eluted with a mobile phase of 0-100% acetonitrile (0.05% AcOH additive) in H₂O (0.05% AcOH additive). The fractions containing pure product were combined, frozen, and lyophilized to afford 0.120 g of INX L-2, 49% yield, as a white solid. LCMS Method A (ESI+): C₅₉H₈₃N₃O₁₅P [M+H]⁺ requires 1104.55, found 1104.5 at 3.894 minutes.

### Synthesis of (S)-4-(2-aminoacetamido)-5-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid TFA salt (INX L-3)

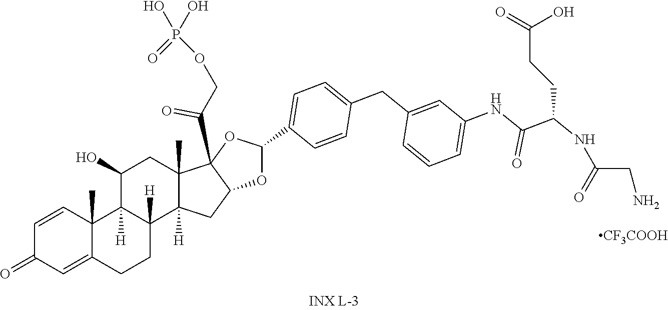

### Procedure

A round bottom flask was charged with tert-butyl ester INX L-2 (0.772 g, 0.7 mmol, 1.0 eq), DCM (10 mL), trifluoroacetic acid (5 mL), and triisopropylsilane (1.2 mL). The mixture was allowed to stir for 8 h at room temperature. Starting material consumption was confirmed by LCMS and the solvent was reduced. The resulting residue was dissolved in DMF (4 mL), loaded onto an Isco C18 Aq 100 g reverse phase column, and eluted with a mobile phase of 0-100% acetonitrile (0.05% TFA additive) in H₂O (0.05% TFA additive). The fractions containing pure product were combined, frozen, and lyophilized to afford 0.3976 g of INX L-3. TFA, 54% yield, as a white solid. LCMS Method A (ESI+): C₄₂H₅₁N₃O₁₃P [M+H]⁺ requires 836.3, found 836.3 at 2.053 minutes.

### Synthesis of (S)-4-(2-(2-bromoacetamido)acetamido)-5-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid (INX L)

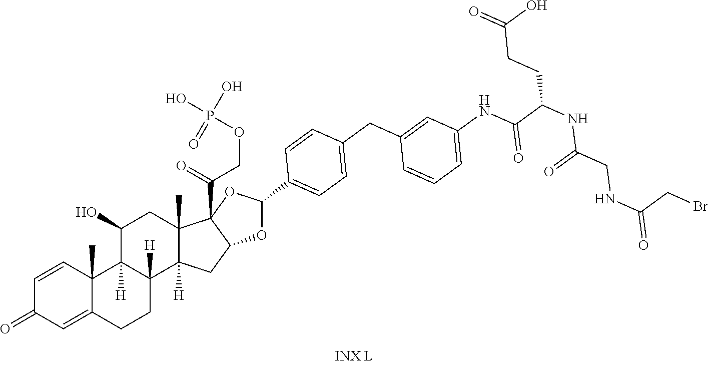

### Procedure

A round bottom flask was charged with 2-bromoacetic acid (0.0250 g, 0.180 mmol, 3.5 eq), DMF (0.50 mL), (7-azabenzotriazol-1-yloxy)tripyrrolidinophosphonium hexafluorophosphate (0.0470 g, 0.090 mmol, 1.7 eq), and N,N-diisopropylethylamine (0.0155 g, 0.120 mmol, 2.3 eq). In a separate vial, amine INX L-3·TFA (0.050 g, 0.052 mmol, 1.0 eq) was dissolved in DMF (2.0 mL) and added to the vessel containing the bromoacetic acid and coupling agent. The mixture was allowed to stir for 30 minutes and starting material consumption was confirmed by LCMS. The crude mixture was purified by preparative HPLC with a mobile phase of 0-100% acetonitrile (0.05% AcOH additive) in H₂O (0.05% AcOH additive). The fractions containing pure product were combined, frozen, and lyophilized to afford 0.030 g of INX L, 60% yield, as a white solid. LCMS Method A (ESI+): C₄₄H₅₂BrN₃O₁₄P [M+H]⁺ requires 956.78, found 956.2 at 2.323 minutes.

### Synthesis of INX-SM-1 and INX N

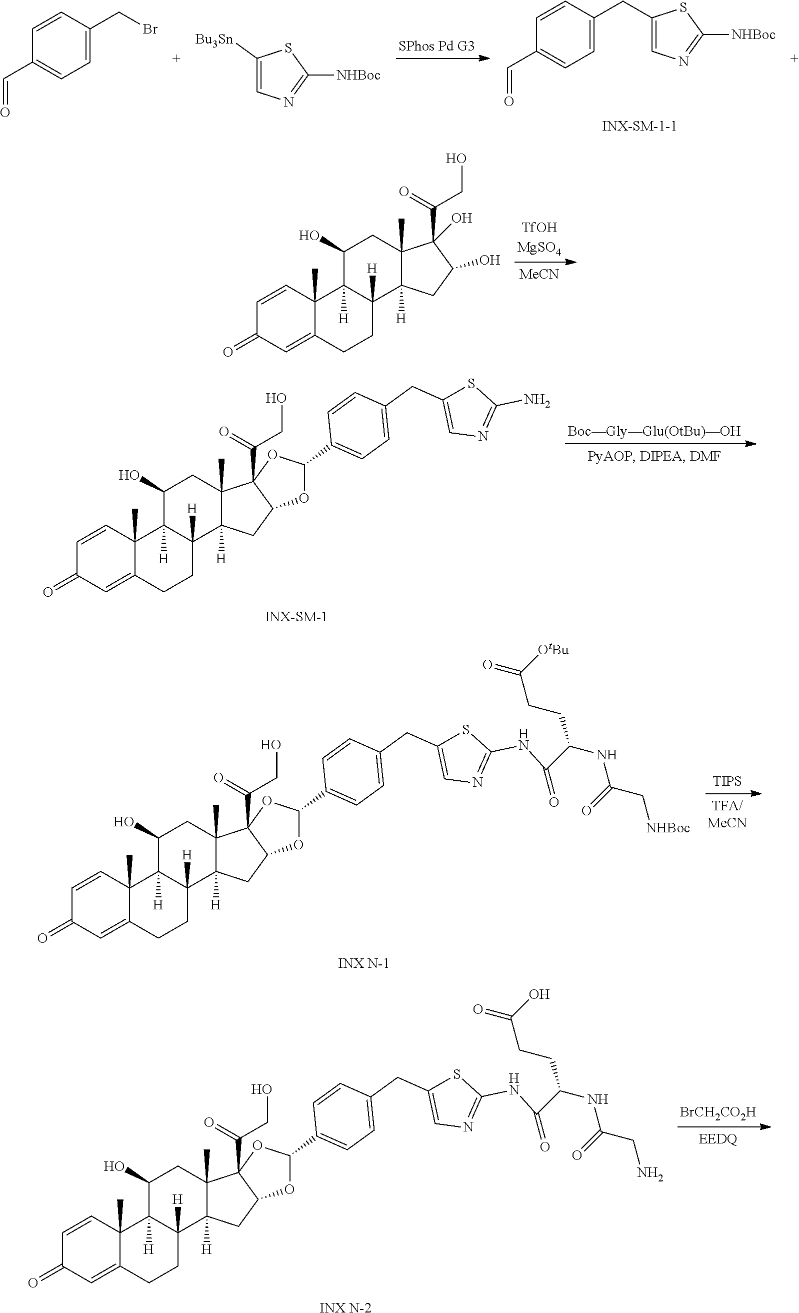

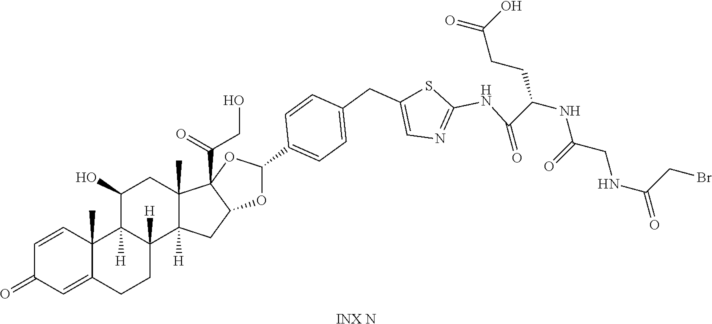

### Synthesis of allyl(S)-(1-((4-(hydroxymethyl)phenyl)amino)-1-oxopropan-2-yl)carbamate (INX-SM-1-1)

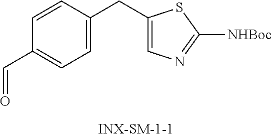 [SMILES: CC(C)(C)OC(=O)Nc1ncc(Cc2ccc(C=O)cc2)s1]

### Procedure

A round bottom flask under inert atmosphere was charged with 4-(Bromomethyl) benzaldehyde (1.465 g, 7.40 mmol, 1.2 eq), tert-butyl (5-(tributylstannyl) thiazol-2-yl)carbamate (3.00 g, 6.10 mmol, 1.0 eq), tripotassium phosphate (3.902 g, 18.40 mmol, 3.0 q), and (2-Dicyclohexylphosphino-2′,6′-dimethoxybiphenyl) [2-(2′-amino-1,1′-biphenyl)]palladium (II) methanesulfonate (1.148 g, 1.50 mmol, 20 mol %). Water (10 mL) and THF (100 mL) were degassed and then added and the mixture was refluxed overnight. Upon completion, which was determined via LCMS, the mixture was cooled to room temperature, reduced, and loaded onto an Isco C18 Aq 450 g reverse phase column and eluted with a mobile phase of 0-100% acetonitrile (10 mM NH₄OAc additive) in H₂O (10 mM NH₄OAc additive). The fractions containing pure product were combined, frozen, and lyophilized to afford 1.064 g of INX-SM-1-1, 55% yield, as an off-white solid. LCMS Method B (ESI+): C₁₁H₁₁N₂OS [M−Boc+H]⁺ requires 219.10, found 219.04 at 1.66 minutes.

### Synthesis of (6aR,6bS,7S,8aS,8bS,10R,11aR,12aS,12bS)-10-(4-((2-aminothiazol-5-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one AcOH salt (INX-SM-1)

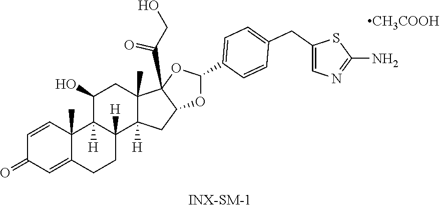

### Procedure

A round bottom flask was charged with 16-α-hydroxyprednisolone (1.1833 g, 3.143 mmol, 1.0 eq), aldehyde INX-SM-1-1 (1.10 g, 3.458 mmol 1.1 eq), and MgSO₄ (1.1355 g, 9.431 mmol, 3.0 eq). The solids were suspended in acetonitrile (31 mL) and the mixture was cooled to 0° C., whereupon trifluoromethanesulfonic acid (1.4 mL, 15.718 mmol, 5.0 eq) was added dropwise. After 10-20 minutes, the reaction turned pink, and the starting material was consumed after 1 h. The solvent was reduced, the crude was loaded onto to an Isco C18 Aq 275 g reverse phase column and eluted with a mobile phase of 0-100% acetonitrile (0.05% AcOH additive) in H₂O (0.05% AcOH additive). The fractions containing pure product were combined, frozen, and lyophilized to afford 1.059 g of INX-SM-1·AcOH, 53% yield, as a white solid. LCMS Method B (ESI+): C₃₂H₃₇N₂O₆S [M+H]⁺ requires 577.23, found 577.93 at 1.10 minutes.

### Synthesis of tert-butyl(S)-4-(2-((tert-butoxycarbonyl)amino)acetamido)-5-((5-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) thiazol-2-yl)amino)-5-oxopentanoate (INX N-1)

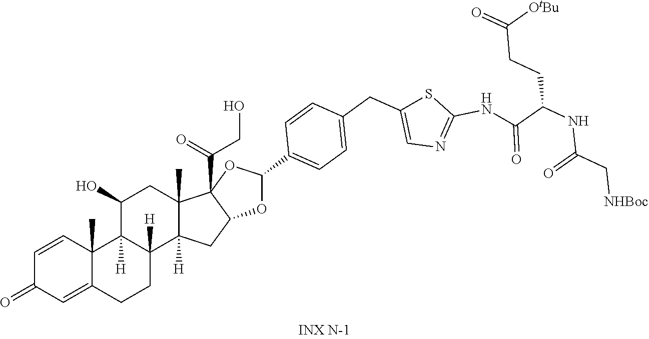

### Procedure

A round bottom flask was charged with INX-SM-1·AcOH (1.000 g, 1.57 mmol, 1.0 eq), Boc-Gly-Glu(OtBu)-OH (3.1212 g, 8.607 mmol, 5.5 eq), and PyAOP (4.5210 g, 8.678 mmol, 5.5 eq). A mixture of 1:1 DCM/DMF (22 mL total volume) was added, followed by DIPEA (3.0 mL, 17.356 mmol, 11.0 eq) and the mixture was stirred for 5 hours. Once most of INX-SM-1 was consumed, the solvent was reduced (to just DMF) and the crude mixture was loaded onto an Isco C18 Aq 275 g reverse phase column and eluted with a mobile phase of 5-100% acetonitrile (0.05% AcOH additive) in H₂O (0.05% AcOH additive). The fractions containing pure product were combined, frozen, and lyophilized to afford 0.4050 g of INX N-1, 28% yield, as a white solid. LCMS Method A (ESI+): C₄₈H₆₃N₄O₁₂S [M+H]⁺ requires 919.41, found 919.4 at 3.089 minutes.

### Synthesis of (S)-4-(2-aminoacetamido)-5-((5-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) thiazol-2-yl)amino)-5-oxopentanoic acid TFA salt (INX N-2)

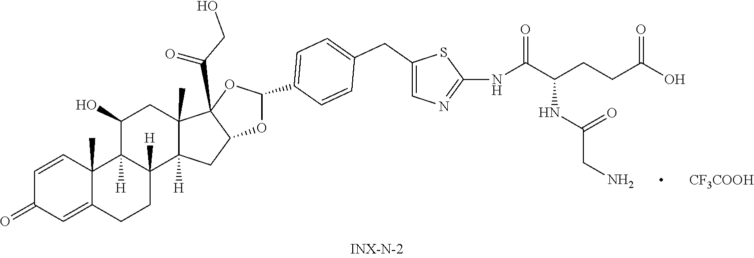

### Procedure

A round bottom flask was charged with tert-butyl ester INX N-1 (0.200 g, 0.2177 mmol, 1.0 eq), MeCN (2.0 mL), trifluoroacetic acid (2.0 mL), and triisopropylsilane (0.70 mL, 3.266 mmol, 15.0 eq). The mixture was allowed to stir for 3 h at room temperature. Starting material consumption was confirmed by LCMS and the solvent was reduced. The resulting residue loaded onto an Isco C18 Aq 30 g reverse phase column and eluted with a mobile phase of 0-100% acetonitrile (0.10% TFA additive) in H₂O (0.10% TFA additive). The fractions containing pure product were combined, frozen, and lyophilized to afford 0.0954 g of INX N-2.TFA, 50% yield, as a white solid. LCMS Method A (ESI+): C₃₉H₄₇N₄O₁₀S [M+H]⁺ requires 763.29, found 763.3 at 1.732 minutes.

### Synthesis of (S)-4-(2-(2-bromoacetamido)acetamido)-5-((5-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) thiazol-2-yl)amino)-5-oxopentanoic acid (INX N)

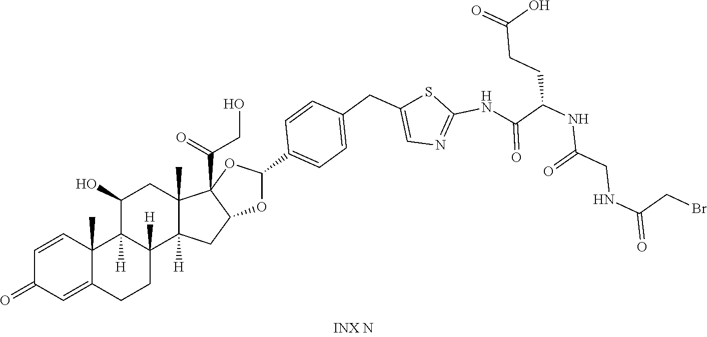

### Procedure

A vial was charged with 2-bromoacetic acid (0.0127 g, 0.0913 mmol, 2.0 eq), which was dissolved in DMF (0.500 mL). N-Ethoxycarbonyl-2-ethoxy-1,2-dihydroquinoline (0.0215 g, 0.0867 mmol, 1.9 eq) was added and the mixture was allowed to stir for 90 minutes. Amine INX N-2·TFA (0.040 g, 0.0457 mmol, 1.0 eq) was then added to the solution along with sodium bicarbonate (0.0230 g, 0.2739 mmol, 6.0 eq) and the mixture was allowed to stir for 2 h (until all INX N-2 was consumed). Once reaction completion was confirmed by LCMS, the crude mixture was directly loaded onto an Isco C18 Aq 15.5 g reverse phase column and eluted with a mobile phase of 0-100% acetonitrile (0.05% AcOH additive) in H₂O (0.05% AcOH additive). The fractions containing pure product were combined, frozen, and lyophilized to afford 0.0091 g of INX N, 22% yield, as a fluffy yellow solid. LCMS Method A (ESI+): C₄₁H₄₈BrN₄O₁₁S [M+H]⁺ requires 883.21, found 883.2 at 2.247 minutes.

### Synthesis of INX-SM-2 and INX Q

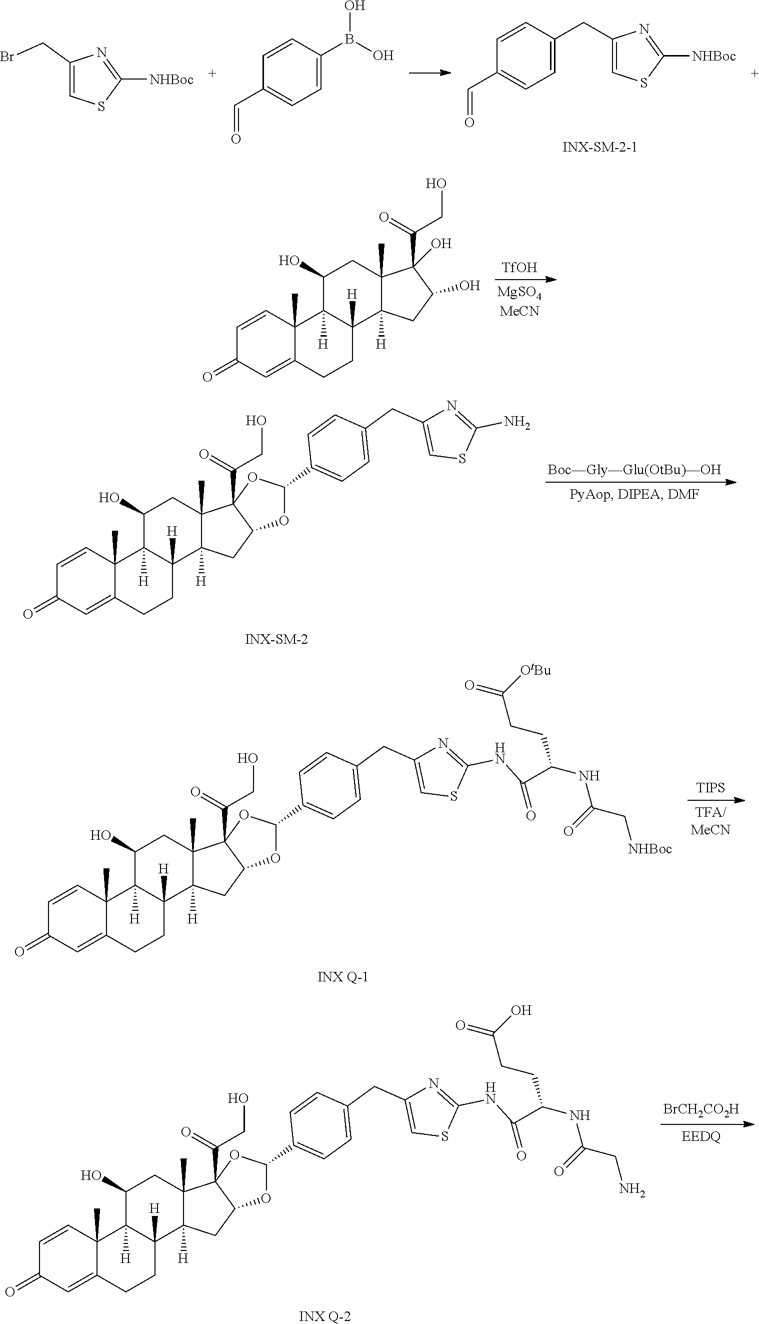

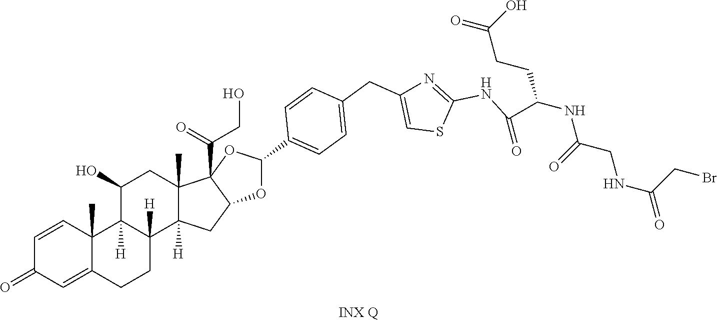

### Synthesis of tert-butyl (4-(4-formylbenzyl) thiazol-2-yl)carbamate (INX-SM-2-1)

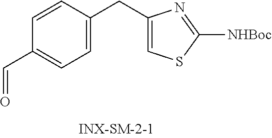 [SMILES: CC(C)(C)OC(=O)Nc1nc(Cc2ccc(C=O)cc2)cs1]

### Procedure

A round bottom flask was back-filled with argon and charged with tert-butyl (4-(bromomethyl)thiazol-2-yl)carbamate (0.150 g, 0.5115 mmol, 1.5 eq), (4-formylphenyl)boronic acid (0.0511 g, 0.3411 mmol, 1.0 eq), potassium carbonate (0.2357 g, 1.706 mmol, 5.0 eq), and [1,1′-Bis(diphenylphosphino)ferrocene]dichloropalladium-dichloromethane complex (0.0279 g, 0.0341 mmol, 0.10 eq). Anhydrous THF (2.5 mL) was added to the flask, which was then equipped with a reflux condenser and heated to 80° C. for 16 h. Starting material consumption was confirmed by LCMS and the mixture was then cooled, diluted with water (10 mL), added to a separatory funnel, and extracted with EtOAc (3×20 mL). The combined organic extracts were dried over Na₂SO₄, filtered, reduced, and loaded onto an Isco Rf Gold 24 g SiO₂ column and eluted with a mobile phase of 0-100% EtOAc in hexanes. The fractions containing pure product were combined and reduced to afford 0.0082 g of compound IN-SM-2-1, 8% yield, as a clear oil which crystallized overnight after removal from reduced pressure. LCMS Method A (ESI+): C₁₆H₁₉N₂O₃S [M+H]⁺ requires 319.10, found 319.1 at 2.1716 minutes.

### Synthesis of (6aR,6bS,7S,8aS,8bS,10R,11aR,12aS,12bS)-10-(4-((2-aminothiazol-4-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one AcOH salt (INX-SM-2)

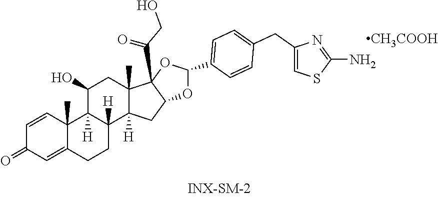

### Procedure

A round bottom flask was charged with 16-α-hydroxyprednisolone (0.1936 g, 0.5143 mmol, 1.0 eq), aldehyde INX-SM-2-1 (0.1800 g, 0.5659 mmol 1.1 eq), and MgSO₄ (0.1857 g, 1.5428 mmol, 3.0 eq). The solids were suspended in acetonitrile (5.1 mL) and the mixture was cooled to 0° C., whereupon trifluoromethanesulfonic acid (0.23 mL, 2.571 mmol, 5.0 eq) was added dropwise. After 10-20 minutes, the reaction turned pinkish and the starting material was consumed after ˜1 h. The solvent was reduced, the crude was loaded onto to an Isco C18 Aq 30 g reverse phase column, and eluted with a mobile phase of 0-100% acetonitrile (0.05% AcOH additive) in H₂O (0.05% AcOH additive). The fractions containing pure product were combined, frozen, and lyophilized to afford 0.1680 g of INX-SM-2·AcOH, 52% yield, as a white solid. LCMS Method A (ESI+): C₃₂H₃₇N₂O₆S [M+H]⁺ requires 577.23, found 577.3 at 1.974 minutes.

### Synthesis of tert-butyl(S)-4-(2-((tert-butoxycarbonyl)amino)acetamido)-5-((4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) thiazol-2-yl)amino)-5-oxopentanoate (INX Q-1)

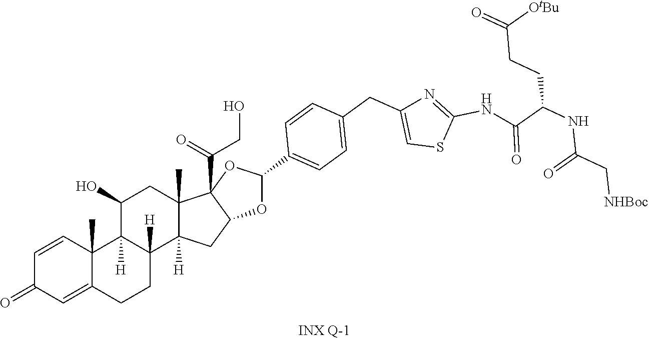

### Procedure

A round bottom flask was charged with INX-SM-2·AcOH (0.1125 g, 0.176 mmol, 1.0 eq), Boc-Gly-Glu(OtBu)-OH (0.0700 g, 0.1760 mmol, 1 eq), and PyAOP (0.1220 g, 0.2340 mmol, 1.3 eq). DMF (1.6 mL) was added, followed by DIPEA (0.081 mL, 0.4686 mmol, 2.6 eq) and the mixture was stirred at room temperature for 2 hours. Once most of INX-SM-2 was consumed, the crude mixture was loaded onto an Isco C18 Aq 15.5 g reverse phase column and eluted with a mobile phase of 0-100% acetonitrile (0.05% AcOH additive) in H₂O (0.05% AcOH additive). The fractions containing pure product were combined, frozen, and lyophilized to afford 0.060 g of INX Q-1, 37% yield, as a white solid. LCMS Method A (ESI+): C₄₈H₆₃N₄O₁₂S [M+H]⁺ requires 919.41, found 919.4 at 2.931 minutes.

### Synthesis of (S)-4-(2-aminoacetamido)-5-((4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) thiazol-2-yl)amino)-5-oxopentanoic acid TFA salt (INX Q-2)

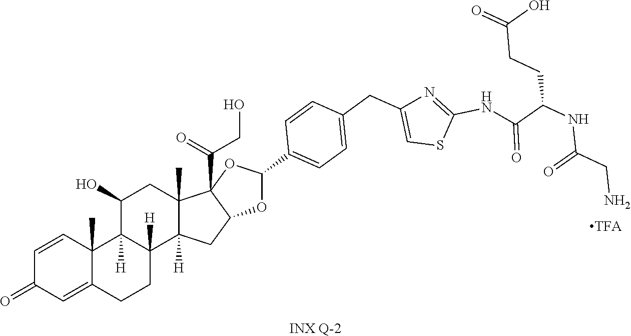

### Procedure

A round bottom flask was charged with tert-butyl ester INX Q-1 (0.0800 g, 0.0871 mmol, 1.0 eq), MeCN (1.0 mL), trifluoroacetic acid (1.0 mL), and triisopropylsilane (0.178 mL, 0.871 mmol, 10.0 eq). The mixture was allowed to stir for 3 h at room temperature. Starting material consumption was confirmed by LCMS and the solvent was reduced. The resulting residue loaded onto an Isco C18 Aq 15.5 g reverse phase column and eluted with a mobile phase of 0-100% acetonitrile (0.10% TFA additive) in H₂O (0.10% TFA additive). The fractions containing pure product were combined, frozen, and lyophilized to afford 0.0100 g of INX Q-2. TFA, 13% yield, as a white solid. LCMS Method A (ESI+): C₃₉H₄₇N₄O₁₀S [M+H]⁺ requires 763.29, found 763.2 at 1.945 minutes.

### Synthesis of (S)-4-(2-(2-bromoacetamido)acetamido)-5-((4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) thiazol-2-yl)amino)-5-oxopentanoic acid (INX Q)

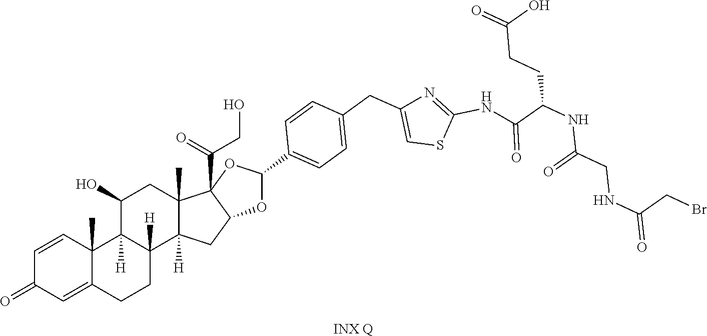

### Procedure

A vial was charged with 2-bromoacetic acid (0.0036 g, 0.0262 mmol, 2.3 eq), which was dissolved in DMF (0.500 mL). N-Ethoxycarbonyl-2-ethoxy-1,2-dihydroquinoline (0.0062 g, 0.0250 mmol, 2.2 eq) was added and the mixture was allowed to stir for 90 minutes. Amine INX Q-2.TFA (0.010 g, 0.0114 mmol, 1.0 eq) was then added to the solution along with sodium bicarbonate (0.0066 g, 0.0786 mmol, 6.9 eq) and the mixture was allowed to stir for 2 h (until all INX Q-2 was consumed). Once reaction completion was confirmed by LCMS, the crude mixture was directly loaded onto an Isco C18 Aq 5.5 g reverse phase column and eluted with a mobile phase of 0-100% acetonitrile (0.05% AcOH additive) in H₂O (0.05% AcOH additive). The fractions containing pure product were combined, frozen, and lyophilized to afford 0.0036 g of INX Q, 36% yield, as a fluffy yellow solid. LCMS Method B (ESI+): C₄₁H₄₈BrN₄O₁₁S [M+H]⁺ requires 883.21, found 883.53 at 1.20 minutes.

### Synthesis of INX-SM-3 & INX-SM-53

### Reaction Scheme

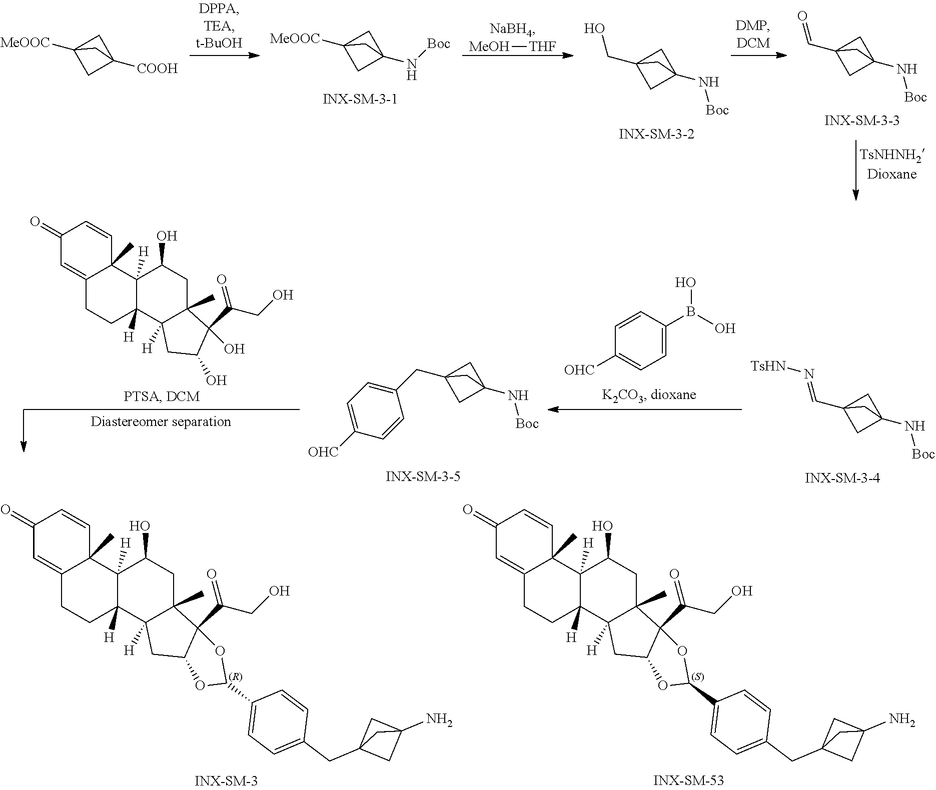

### Synthesis of methyl 3-((tert-butoxycarbonyl)amino)bicyclo[1.1.1]pentane-1-carboxylate (INX-SM-3-1)

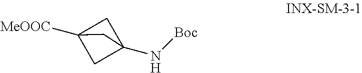 [SMILES: COC(=O)C12CC(NC(=O)OC(C)(C)C)(C1)C2]

### Procedure

To a solution of 3-(methoxycarbonyl) bicyclo[1.1.1]pentane-1-carboxylic acid (10 g, 58.76 mmol) in tert-Butyl alcohol (20 mL) diphenylphosphoryl azide (DPPA) (20.2 mL, 88.15 mmol) and triethyl amine (33.04 mL, 235.0 mmol) was added at room temperature. The reaction mixture was heated at 80° C. for 1 h. After completion of reaction as indicated by TLC, reaction mixture was poured into water and extracted with ethyl acetate. The combined organic layer was dried over Na₂SO₄ and evaporated under vacuum to give crude product. The crude was purified by silica gel column chromatography (ethyl acetate/hexane: 12:88) to give INX-SM-3-1 as white solid (10 g, 70.55%). ¹H NMR (CDCl3) δ: 7.43 (bs, 1H), 3.69 (s, 3H), 2.30 (s, 6H), 1.46 (s, 9H).

### Synthesis of tert-butyl (3-(hydroxymethyl)bicyclo[1.1.1]pentan-1-yl)carbamate (INX-SM-3-2)

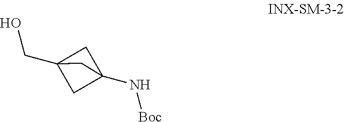 [SMILES: CC(C)(C)OC(=O)NC12CC(CO)(C1)C2]

### Procedure

To a stirred solution of methyl 3-((tert-butoxycarbonyl)amino)bicyclo[1.1.1]pentane-1-carboxylate (INX-SM-3-1) (5 g, 20.70 mmol) in THF:MeOH (3:1) (20 mL), sodium borohydride (3.9 g, 103.5 mmol) was added at room temperature and stirred for another 16 h. After completion of reaction as indicated by TLC, reaction mixture was quenched with dil. aqueous HCl solution and extracted with ethyl acetate. The combined organic layer was dried over Na₂SO₄ and evaporated under vacuum to yield INX-SM-3-2 crude product (4.3 g, 97.38%). LCMS: 214.0 [M+H]⁺; ¹H NMR (CDCl3) δ: 4.99 (bs, 1H), 3.72 (s, 2H), 1.95 (s, 6H), 1.42 (s, 6H).

### Synthesis of tert-butyl (3-formylbicyclo[1.1.1]pentan-1-yl)carbamate (INX-SM-3-3)

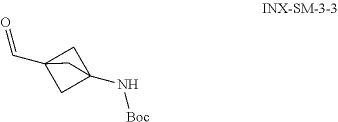 [SMILES: CC(C)(C)OC(=O)NC12CC(C=O)(C1)C2]

### Procedure

To a stirred solution of tert-butyl (3-(hydroxymethyl)bicyclo[1.1.1]pentan-1-yl)carbamate (INX-SM-3-2) (0.1 g, 0.46 mmol) in DCM (2 mL), Dess-Martin periodinane (DMP) (0.40 g, 40.93 mmol) was added at room temperature and stirred for 30 min. After completion of reaction as indicated by TLC, reaction mixture was quenched with saturated NaHCO₃ solution and extracted with ethyl acetate. The combined organic layer was dried over Na₂SO₄ and evaporated under vacuum to give crude product. The crude was purified by silica gel column chromatography (ethyl acetate/hexane: 40:60) to give INX-SM-3-3 as white solid (0.050 g, 52%). ¹H NMR (DMSO-d6) δ: 9.59 (s, 1H), 7.68 (bs, 1H), 2.12 (s, 6H), 1.37 (s, 9H).

### Synthesis of tert-butyl (3-((2-tosylhydrazono)methyl)bicyclo[1.1.1]pentan-1-yl) carbamate (INX-SM-3-4)

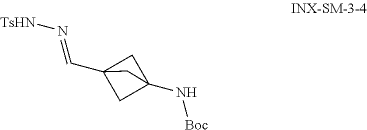

### Procedure

To a stirred solution of tert-butyl (3-formylbicyclo[1.1.1]pentan-1-yl)carbamate (INX-SM-3-3) (0.40 g, 1.89 mmol) in dioxane (5 mL), p-toluenesulfonhydrazide (8.8 g, 47.20 mmol) was added and stirred for 2 h at 50° C. After completion of reaction as indicated by TLC, reaction mixture was poured into water and extracted with ethyl acetate. The combined organic layer was dried over Na₂SO₄ and evaporated under vacuum to give crude product. The crude was purified by silica gel column chromatography (ethyl acetate/hexane: 30:70) to give INX-SM-3-4 as white solid (0.28 g, 38.96%). LCMS: 324.5 (M-56); ¹H NMR (DMSO-d6) δ: 11.07 (s, 1H), 7.66 (d, J=8 Hz, 2H), 7.40 (d, J=8 Hz, 2H), 7.23 (s, 1H), 2.38 (s, 3H), 1.90 (s, 6H), 1.36 (s, 9H).

### Synthesis of tert-butyl (3-(4-formylbenzyl)bicyclo[1.1.1]pentan-1-yl)carbamate (INX-SM-3-5)

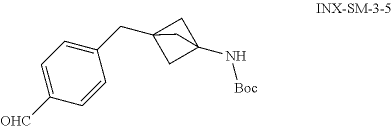 [SMILES: CC(C)(C)OC(=O)NC12CC(Cc3ccc(C=O)cc3)(C1)C2]

### Procedure

To a stirred solution of tert-butyl)-(3-((2-tosylhydrazono) methyl)bicyclo[1.1.1]pentan-1-yl)carbamate (INX-SM-3-4) (3.20 g, 8.43 mmol) in dioxane (30 mL), (4-formylphenyl)boronic acid (1.64 g, 8.43 mmol) and K₂CO3 (1.74 g, 12.64 mmol) was added at room temperature and stirred for another 2 h at 110° C. After completion of reaction as indicated by TLC, reaction mixture was poured into water and extracted with ethyl acetate. The combined organic layer was dried over Na₂SO₄ and evaporated under vacuum to give crude product. The crude was purified by silica gel column chromatography (ethyl acetate/hexane: 15:85) to give INX-SM-3-5 as white solid (0.81 g, 31.87%). LCMS: 302.5 (M+H)⁺; ¹H NMR (DMSO-d6) δ: 9.97 (s, 1H), 7.84 (d, J=7.6 Hz, 2H), 7.33 (d, J=7.6 Hz, 2H), 2.89 (s, 2H), 1.68 (s, 6H), 1.33 (s, 9H).

### Synthesis of (6aR,6bS,7S,8aS,8bS,10R,11aR,12aS,12bS)-10-(4-((3-aminobicyclo[1.1.1]pentan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-3); and (6aR,6bS,7S,8aS,8bS,10S,11aR,12aS,12bS)-10-(4-((3-aminobicyclo[1.1.1]pentan-1-yl)methyl)phenyl)-7-hydroxy-8b-(2-hydroxyacetyl)-6a,8a-dimethyl-1,2,6a,6b,7,8,8a,8b, 11a,12,12a,12b-dodecahydro-4H-naphtho[2′,1′:4,5]indeno[1,2-d][1,3]dioxol-4-one (INX-SM-53)

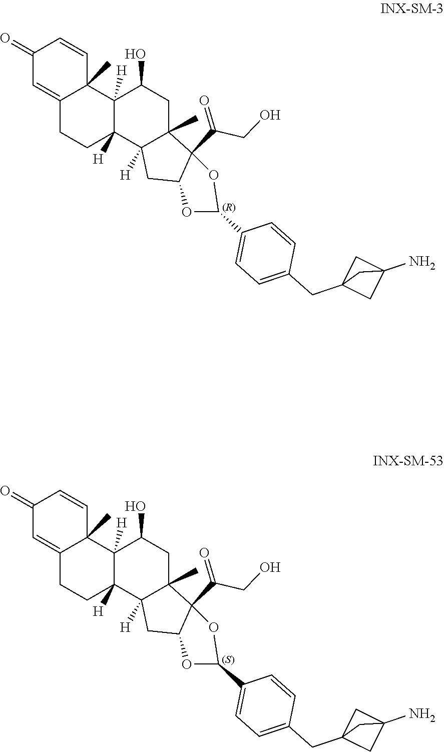

To a solution of tert-butyl (3-(4-formylbenzyl)bicyclo[1.1.1]pentan-1-yl)carbamate (INX-SM-3-5) (1.0 g, 3.31 mmol) and (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (16-α-hydroxyprednisolone) (1.24 g, 3.31 mmol) in DCM (10 mL), PTSA (0.95 g, 4.97 mmol) was added and stirred for another 16 h at room temperature. After completion of reaction as indicated by TLC, reaction mixture was poured into water and extracted with ethyl acetate. The combined organic layer was dried over Na₂SO₄ and evaporated under vacuum to give the crude product as mixture of isomers. The crude was purified by prep-HPLC and then the isomers were separated by chiral prep-HPLC (Column: IG 250*21 μm, 5 micron, Mobile phase: A=0.1% ammonia in Heptane, B=IPA:ACN (70:30), A:B=60:40) to give Isomer-1 and Isomer-2. These isomers were eluted at retention time 6.72 min (Isomer-1) and 11.87 min (Isomer-2).
INX-SM-3 (Isomer-1): LCMS: 561.0 (M+H)⁺; ¹H NMR (400 MHZ, MeOD, Key proton assignment): δ: 5.45 (s, 1H, Acetal-H), 5.07 (d, J=5.2 Hz, 1H, C16H)
INX-SM-53 (Isomer-2): LCMS 561.1 (M+H)⁺; ¹H NMR (400 MHZ, MeOD, Key proton assignment): δ: 6.13 (s, 1H, Acetal-H), 5.41 (d, J=5.6 Hz, 1H, C16H)

### Synthesis of (S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicycle[1.1.1]pentan-1-yl)amino)-5-oxopentanoic acid (INX—P)

### Reaction Scheme

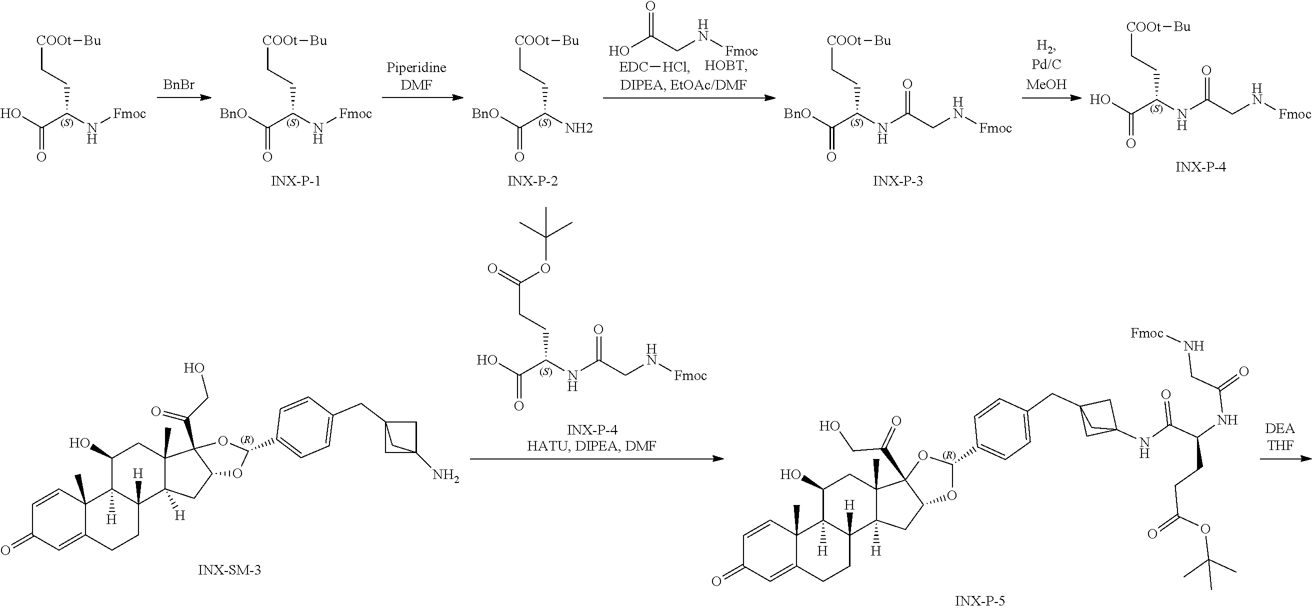

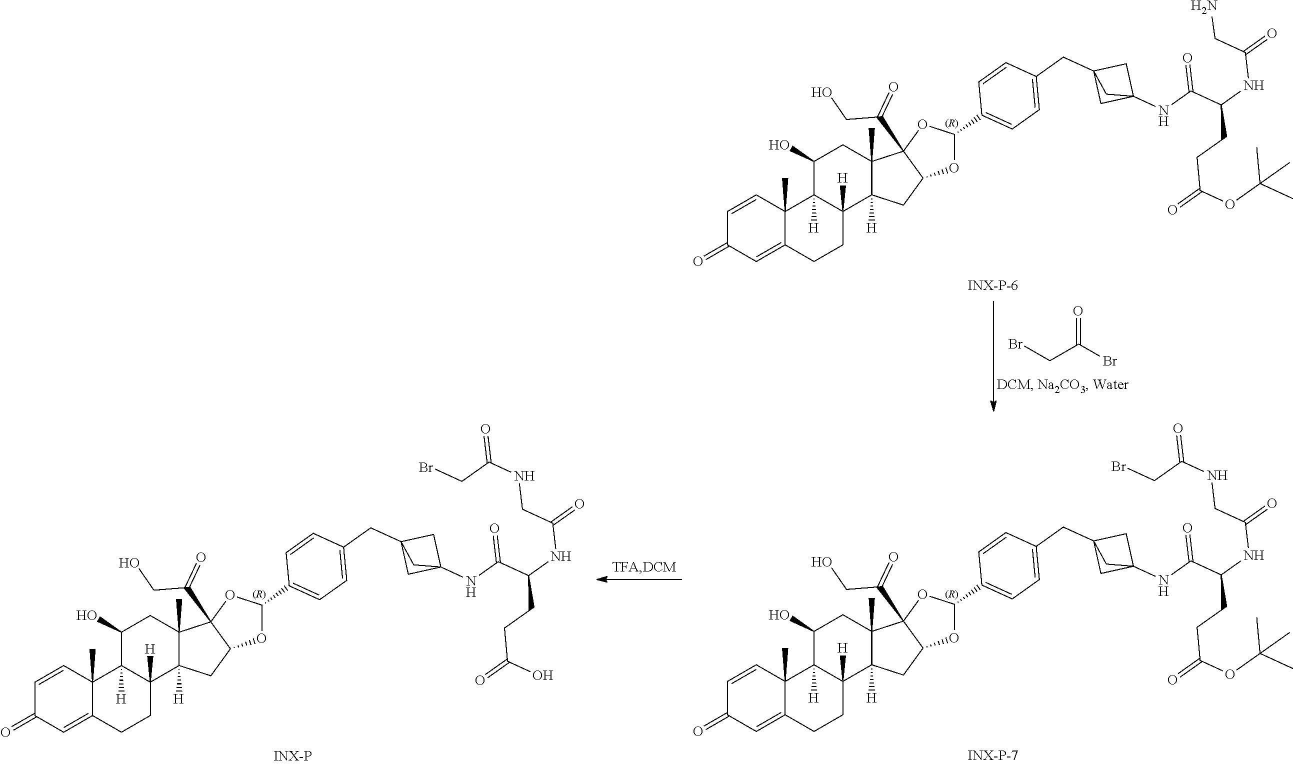

### Synthesis of 1-benzyl 5-(tert-butyl) (((9H-fluoren-9-yl)methoxy)carbonyl)-L-glutamate

### (INX-P-1)

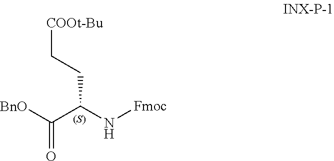

### Procedure

A 500 mL three-necked round bottom flask was charged with(S)-2-((((9H-fluoren-9-yl)methoxy)carbonyl)amino)-5-(tert-butoxy)-5-oxopentanoic acid (25 g, 58.82 mmol) and sodium bicarbonate (9.8 g, 116.66 mmol) in DMF (200 mL). To this suspension, benzyl bromide (10.9 g, 63.74 mmol) was added at room temperature and stirred for 16 h at room temperature. After completion of reaction as indicated by TLC, reaction mixture was poured into water and extracted with ethyl acetate. The combined organic layer was washed with water, dried over Na₂SO₄ and evaporated under vacuum. The crude was triturated with diethyl ether and pentane to give INX P-1 as white solid (26 g, 85.83%). LCMS: 516.4 (M+H)⁺.

### Synthesis of 1-benzyl 5-(tert-butyl) L-glutamate (INX-P-2)

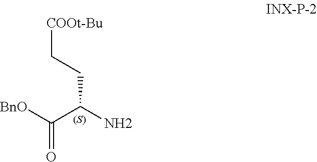

### Procedure

A 500 mL single-necked round bottom flask was charged with 1-benzyl 5-(tert-butyl) (((9H-fluoren-9-yl)methoxy)carbonyl)-L-glutamate (INX-P-1) (26 g, 50.42 mmol) and THF (200 mL). To this solution, diethyl amine (36.8 g, 504.11 mmol) was added and stirred for 3 h at room temperature. After completion of reaction as indicated by TLC, reaction mixture was poured into water and extracted with ethyl acetate. The combined organic layer was dried over Na₂SO₄, evaporated under vacuum and triturated with pentane to give INX-P-2 as light-yellow sticky (28 g). Crude product was directly used for next step without any analytical data.

### Synthesis of 1-benzyl 5-(tert-butyl) (((9H-fluoren-9-yl)methoxy)carbonyl) glycyl-L-glutamate (INX-P-3)

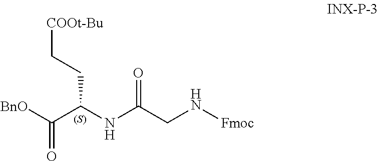

### Procedure

A 500 mL single-necked round bottom flask was charged with (((9H-fluoren-9-yl)methoxy)carbonyl) glycine (28.0 g, 94.27 mmol) and DMF (200 mL). To this solution, EDC·HCl (19.7 g, 102.76 mmol), HOBT (13.9 g, 102.76 mmol), DIPEA (24.2 g, 187.24 mmol) and 1-benzyl 5-(tert-butyl) L-glutamate (INX-P-2) (30.38 g, 103.25 mmol) were added at room temperature and stirred for 1 h. After completion of reaction as indicated by TLC, reaction mixture was quenched with water and extracted with ethyl acetate. The combined organic layer was dried over Na₂SO₄ and evaporated under vacuum to give crude product. The crude was purified by column chromatography (ethyl acetate/hexane, 50:50) to give INX-P-3 as light yellow (12.0 g, 23.64%). LCMS: 574.4 (M+H)⁺.

### Synthesis of (S)-2-(2-((((9H-fluoren-9-yl)methoxy)carbonyl)amino)acetamido)-5-(tert-butoxy)-5-oxopentanoic acid (INX-P-4)

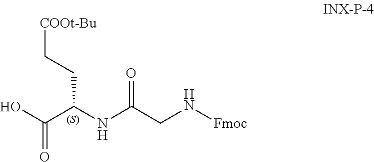

### Procedure

A 500 mL single-necked round bottom flask was charged with 1-benzyl 5-(tert-butyl) (((9H-fluoren-9-yl)methoxy)carbonyl) glycyl-L-glutamate (INX-P-3) (12.0 g, 20.95 mmol) in MeOH (120 mL). To this solution, 10% Pd/C (2.4 g) was added at room temperature and purged with hydrogen for 3-4h. After completion of reaction as indicated by TLC, reaction mixture was filtered through a bed of celite and the filtrate was evaporated under vacuum. The crude was purified by reverse phase column chromatography (acetonitrile/water) to give INX-P-4 as off white solid (5 g, 49.45%). LCMS: 483.2 (M+H)⁺.

### Synthesis of tert-butyl(S)-4-(2-((((9H-fluoren-9-yl)methoxy)carbonyl)amino)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-P-5)

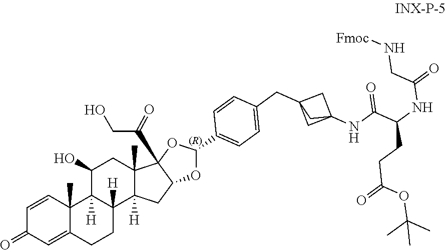

### Procedure

A 50 mL single-necked round bottom flask was charged with(S)-2-(2-((((9H-fluoren-9-yl)methoxy)carbonyl)amino)acetamido)-5-(tert-butoxy)-5-oxopentanoic acid (INX-P-4) (0.47 g, 0.97 mmol), HATU (0.55 g, 1.45 mmol), DIPEA (0.25 g, 1.94 mmol) and DMF (4 mL) at room temperature. To this solution, (6aR,6bS,7S,8aS,8bS,10R,11aR,12aS,12bS)-10-(4-((3-aminobicyclo[1.1.1]pentan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-3) (0.59 g, 1.06 mmol) was added at room temperature and stirred for 1 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water, 50:50) to give INX-P-5 as light-yellow solid (0.42 g, 57.38%). LCMS: 1025.0 (M+H)⁺.

### Synthesis of tert-butyl(S)-4-(2-aminoacetamido)-5-((3-(4-((6aR,6bS,7S,8aS,8bS, 10R,11aR,12aS,12bS)-7-hydroxy-8b-(2-hydroxyacetyl)-6a,8a-dimethyl-4-oxo-2,4,6a,6b,7,8, 8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-P-6)

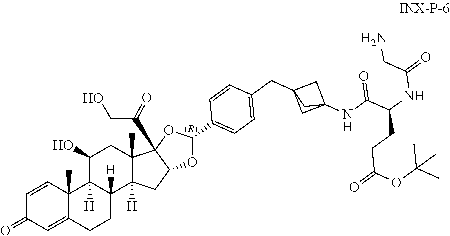

### Procedure

A 50 mL single-necked round bottom flask was charged with tert-butyl(S)-4-(2-((((9H-fluoren-9-yl)methoxy)carbonyl)amino)acetamido)-5-((3-(4-11aR,12aS,12bS)-7-hydroxy-8b-(2-hydroxyacetyl)-6a,8a-((6aR,6bS,7S,8aS,8bS,10R,12,12a,12b-dodecahydro-1H-dimethyl-4-oxo-2,4,6a,6b,7,8,8a,8b,11a, naphtho[2′,1′:4,5]indeno[1,2-d][1,3]dioxol-10-yl)benzyl)bicycle[1.1.1]pentan-1-yl)amino)-5-oxopentanoate (INX-P-5) (0.40 g, 0.41 mmol) and THF (4 mL). To this solution, diethyl amine (0.40 g, 4.10 mmol) was added and stirred for 3 h at room temperature. After completion of reaction as indicated by TLC, reaction mixture was evaporated under vacuum to give INX-P-6 as yellow solid (0.23 g, 73.43%) LCMS: 802.1 (M+H)⁺.

### Synthesis of tert-butyl(S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-P-7)

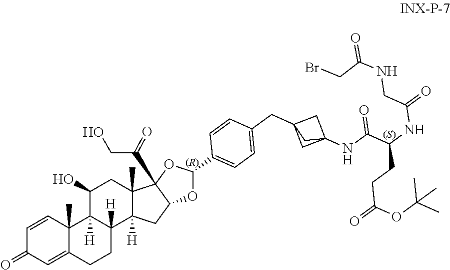

A 25 mL single-necked round bottom flask was charged with tert-butyl(S)-4-(2-aminoacetamido)-5-((3-(4-((6aR,6bS,7S,8aS,8bS,10R,11aR,12aS,12bS)-7-hydroxy-8b-(2-hydroxy acetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-P-6) (0.23 g, 0.28 mmol) and DCM (2 mL). To this solution, Na₂CO₃ (0.12 g, 0.57 mmol) solution in water (1 mL) followed by bromoacetyl bromide (0.029 g, 0.28 mmol) was added at room temperature and stirred for 1 h. After completion of reaction as indicated by TLC, reaction mixture was quenched with water and extracted with DCM. The combined organic layer was dried over Na₂SO₄ and evaporated under vacuum. The crude was purified by reverse phase column chromatography (acetonitrile/water, 50:50) to give INX-P-7 as pale yellow solid (0.090 g, 34.00%). LCMS: 922.9 & 924.8 (M & M+2).

### Synthesis of (S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicycle[1.1.1]pentan-1-yl)amino)-5-oxopentanoic acid (INX—P)

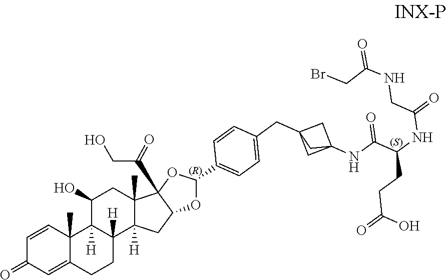

### Procedure

A 10 mL single-necked round bottom flask was charged with tert-butyl(S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxo pentanoate (INX-P-7) (0.090 g, 0.097 mmol) and DCM (2 mL). To this solution, TFA (0.055 g, 0.48 mmol) was added and stirred for 2 h at room temperature. After completion of reaction as indicated by TLC, reaction mixture was evaporated under vacuum. The crude was purified by prep-HPLC to obtain INX—P (Column: SUNFIRE Prep C18 OBD, 19×250 mm, 5 μm, Mobile phase: A=0.1% FA in Water, B=acetonitrile; A:B, 55:45), Retention time 15.51 min to give R-Isomer as off white solid (0.010 g, 11.83%). LCMS: 866.80 & 868.8 (M & M+2); ¹H NMR (400 MHZ, DMOS-d6, Key proton assignment): δ: 5.40 (s, 1H, Acetal-H), 4.92 (d, J=4.8 Hz, 1H, C16H).

### Synthesis of (6aR,6bS,7S,8aS,8bS,10R,11aR,12aS,12bS)-10-(3-(3-aminobenzyl) bicyclo[1.1.1]pentan-1-yl)-7-hydroxy-8b-(2-hydroxyacetyl)-6a,8a-dimethyl-1,2,6a,6b,7,8, 8a,8b,11a,12,12a,12b-dodecahydro-4H-naphtho[2′,1′:4,5]indeno[1,2-d][1,3]dioxol-4-one (INX-SM-4); and

### (6aR,6bS,7S,8aS,8bS,10S,11aR,12aS,12bS)-10-(3-(3-aminobenzyl)bicyclo[1.1.1]pentan-1-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-54)

### Reaction Scheme

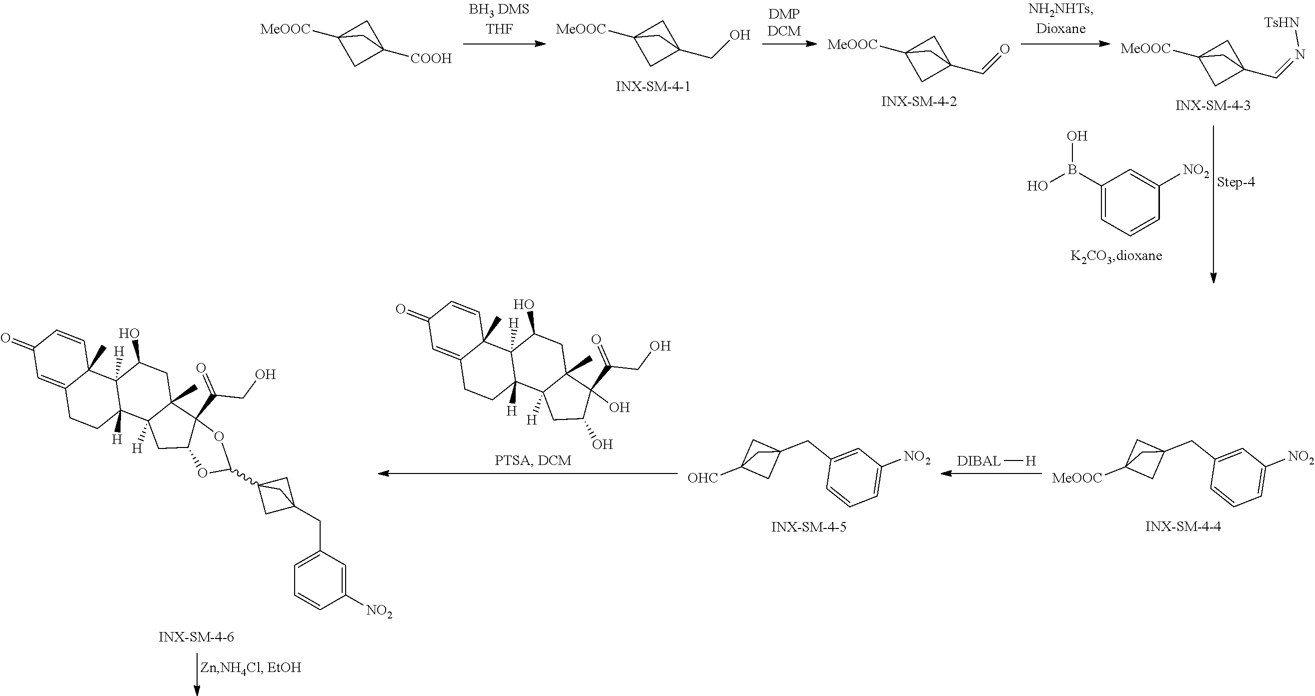

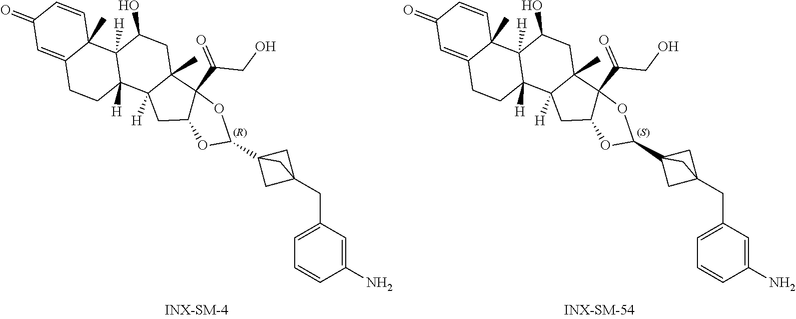

### Synthesis methyl 3-(hydroxymethyl)bicyclo[1.1.1]pentane-1-carboxylate (INX-SM-4-1)

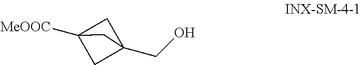 [SMILES: COC(=O)C12CC(CO)(C1)C2]

### Procedure

To a solution of 3-(methoxycarbonyl) bicyclo[1.1.1]pentane-1-carboxylic acid (10 g, 58.75 mmol) in THF (15 mL), borane dimethyl sulfide (BH3·DMS) (13.49 mL, 176.2 mmol) was added drop wise at 0° C. The reaction mixture was allowed to stir at 0° C. for additional 30 min. After completion of reaction as indicated by TLC, reaction mixture was quenched by slow addition of dil. HCl solution. The product was extracted with ethyl acetate and combined organic layer was dried over Na₂SO₄ and evaporated under vacuum to give the title compound as gummy solid (8.2 g, 89.30%). The crude was carried forward in next step. 1H NMR (CDCl3) δ: 3.68 (s, 3H), 3.63 (s, 2H), 3.07 (bs, 1H), 2.05 (s, 6H).

### Synthesis of methyl 3-formylbicyclo[1.1.1]pentane-1-carboxylate (INX-SM-4-2)

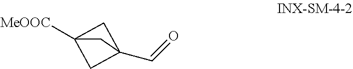 [SMILES: COC(=O)C12CC(C=O)(C1)C2]

### Procedure

To a stirred solution of methyl 3-(hydroxymethyl)bicyclo[1.1.1]pentane-1-carboxylate (INX-SM-4-1) (8.0 g, 56.27 mmol) in DCM (240 mL), Dess-Martin periodinane (DMP) (23.87 g, 56.27 mmol) was added at 0° C. and stirred for another 2 h at room temperature. After completion of reaction as indicated by TLC, reaction mixture was quenched with saturated solution of NaHCO₃. The reaction mixture was extracted with DCM. The combined organic layer was dried over Na₂SO₄ and evaporated under vacuum to give the title compound as gummy white solid (12 g, crude). The crude product was carried forward for next step without purification.

### Synthesis of methyl 3-((2-tosylhydrazono)methyl)bicyclo[1.1.1]pentane-1-carboxylate (INX-SM-4-3)

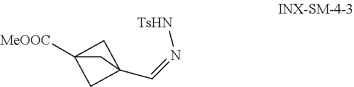

### Procedure

A mixture of methyl 3-formylbicyclo[1.1.1]pentane-1-carboxylate (INX-SM-4-2) (8 g, 51.88 mmol) and p-toluenesulfonyl hydrazide (9.66 g, 51.88 mmol) in dioxane (120 mL) was heated at 50° C. for 2 h. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane: 60:40) to give the title compound as white solid (10 g, 60.34%). LCMS: 323.2 (M+H)⁺; ¹H NMR (DMSO-d6) δ: 11.19 (s, 1H), 7.66 (d, J=8 Hz, 2H), 7.40 (d, J=8 Hz, 2H), 7.20 (s, 1H), 3.60 (s, 3H), 2.38 (s, 3H), 2.09 (s, 6H).

### Synthesis of methyl 3-(3-nitrobenzyl)bicyclo[1.1.1]pentane-1-carboxylate (INX-SM-4-4)

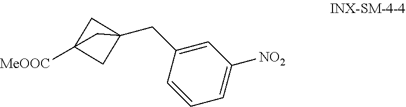 [SMILES: COC(=O)C12CC(Cc3cccc([N+](=O)[O-])c3)(C1)C2]

### Procedure

To a stirred solution of methyl 3-((2-tosylhydrazono)methyl)bicyclo[1.1.1]pentane-1-carboxylate (INX-SM-4-3) (4 g, 12.42 mmol) in dioxane (30 mL), (4-nitrophenyl)boronic acid (2.07 g, 12.42 mmol) and K₂CO3 (2.57 g, 18.63 mmol) was added at room temperature and stirred at 110° C. for 2 h. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane: 06:94) to give title compound as white solid (0.520 g, 16.04%). ¹H NMR (DMSO-d6) δ: 8.10 (d, J=6.4 Hz, 1H), 8.01 (s, 1H), 7.64-7.59 (m, 2H), 3.55 (s, 3H), 2.95 (s, 2H), 1.82 (s, 6H).

### Synthesis of 3-(3-nitrobenzyl)bicyclo[1.1.1]pentane-1-carbaldehyde (INX-SM-4-5)

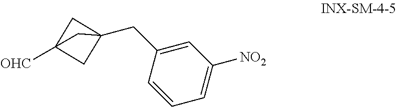 [SMILES: O=CC12CC(Cc3cccc([N+](=O)[O-])c3)(C1)C2]

### Procedure

To a stirred solution of methyl 3-(3-nitrobenzyl)bicyclo[1.1.1]pentane-1-carboxylate (INX-SM-4-4) (0.490 g, 1.87 mmol) in DCM (25 mL), diisobutylaluminium hydride (1M in toluene, 3.2 mL, 3.75 mmol) was added at −78° C. and stirred further for 30 min. After completion of reaction as indicated by TLC, reaction mixture was quenched with dilute HCl solution and allowed to come at room temperature then extracted with DCM. The combined organic layer was washed with brine, dried over Na₂SO₄ and evaporated under vacuum. The crude was purified by silica gel column chromatography (ethyl acetate/hexane: 18:82) to give title compound as white solid (0.27 g, 62.26%). ¹H NMR (DMSO-d6) δ: 9.55 (s, 1H), 8.12 (d, J=8 Hz, 1H), 7.99 (s, 1H), 7.51 (t, J=7.6 Hz, 1H), 7.44 (d, J=7.6 Hz, 1H), 2.95 (s, 2H), 1.93 (s, 6H).

### Synthesis of (6aR,6bS,7S,8aS,8bS,10R,11aR,12aS,12bS)-7-hydroxy-8b-(2-hydroxy acetyl)-6a,8a-dimethyl-10-(3-(3-nitrobenzyl)bicyclo[1.1.1]pentan-1-yl)-1, 2,6a,6b,7,8,8a, 8b,11a,12,12a,12b-dodecahydro-4H-naphtho[2′,1′:4,5]indeno[1,2-d][1,3]dioxol-4-one (INX-SM-4-6)

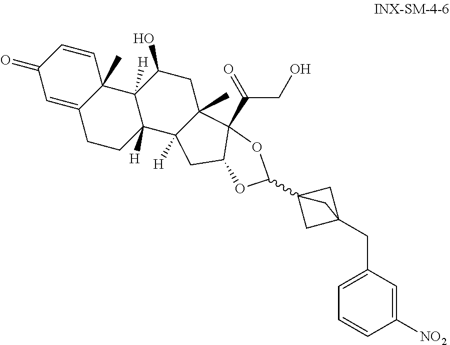

### Procedure

To a stirred solution of 3-(3-nitrobenzyl)bicyclo[1.1.1]pentane-1-carbaldehyde (INX-SM-4-5) (0.27 g, 1.16 mmol) in DCM (30 mL) was added (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dode cahydro-3H-cyclopenta[a]phenanthren-3-one (16-alpha-hydroxyprednisolone) (0.351 g, 0.93 mmol) and p-toluenesulfonic acid (0.30 g, 1.76 mmol). The reaction mixture was stirred for additional 16h at room temperature. After completion of reaction as indicated by TLC, reaction mixture was poured into water and extracted with DCM. The combined organic layer was dried over Na₂SO₄ and evaporated under vacuum to give title compound as mixture of isomer (0.470 g, crude). LCMS: 590.93 (M+H)⁺.

Further the isomers were separated by chiral prep HPLC (Column: IG 250*21 μm, 5 micron, Mobile phase: A=0.1% ammonia in Heptane, B=IPA:ACN (70:30), A:B=75:25) to give Isomer-1 and Isomer-2. These isomers were eluted at retention time 12.85 min (Isomer-1) and 19.40 min (Isomer-2).
Isomer-1: ¹H NMR (400 MHZ, CDCl3) Fr-1: δ 4.94 (d, 1H, C16H), 4.57 (s, 1H, Acetal-H)
Isomer-2: ¹H NMR (400 MHZ, CDCl3) Fr-1: δ 5.19 (d, 1H, C16H), 5.08 (s, 1H, Acetal-H)

### Synthesis of (6aR,6bS,7S,8aS,8bS,10R,11aR,12aS,12bS)-10-(3-(3-aminobenzyl) bicyclo[1.1.1]pentan-1-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-4); and

### (6aR,6bS,7S,8aS,8bS,10S,11aR,12aS,12bS)-10-(3-(3-aminobenzyl)bicyclo[1.1.1]pentan-1-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-54)

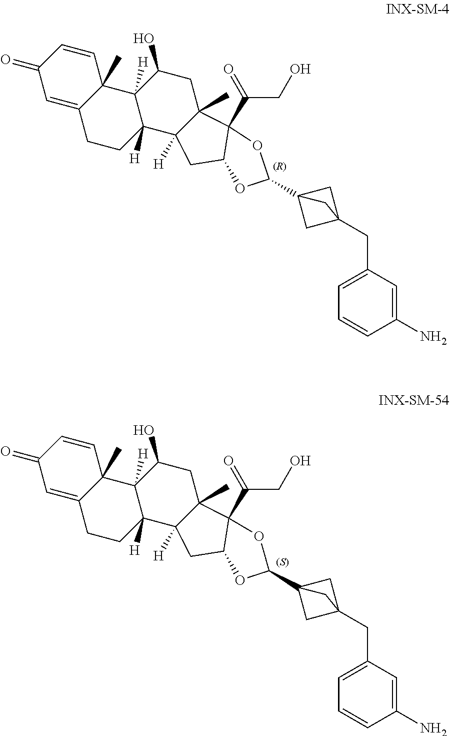

### Procedure

To a stirred solution of (INX-SM-4-6, mix of isomer) (0.30 g, 0.50 mmol) in ethanol (10 mL) was added NH₄Cl (0.22 g, 4.0 mmol) and Zn dust (0.26 g, 4.0 mmol). The reaction mixture was stirred for 2 h at 80° C. After completion of reaction as indicated by TLC, reaction mixture was filtered, and filtrate was evaporated under vacuum to give title compound as mixture of isomer (0.360 g, crude).

Further the isomers were separated by chiral prep HPLC (Column: IG 250*21 μm, 5 micron, Mobile phase: A=0.1% ammonia in Heptane, B=IPA:ACN (70:30), A:B=82:18) to give Isomer-1 and Isomer-2. These isomers were eluted at retention time 27.96 min (Isomer-1) and 43.90 min (Isomer-2).
INX-SM-4 (Isomer-1): LCMS: 560.90 (M+H)⁺; ¹H NMR (400 MHZ, MeOD, Key proton assignment) δ: 5.00-4.90 (m, 2H, acetal & C16-H)
INX-SM-54 (Isomer-2: LCMS: 561.00 (M+H)⁺; ¹H NMR (400 MHZ, MeOD, Key proton assignment) δ: 5.16 (d, J=7.2 Hz, 1H, C16-H), 5.09 (s, 1H, acetal-H)

### Synthesis of S)-4-(2-(2-bromoacetamido)acetamido)-5-((3-((3-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) bicyclo[1.1.1]pentan-1-yl)methyl)phenyl)amino)-5-oxopentanoic acid (INX Q)

### Reaction Scheme

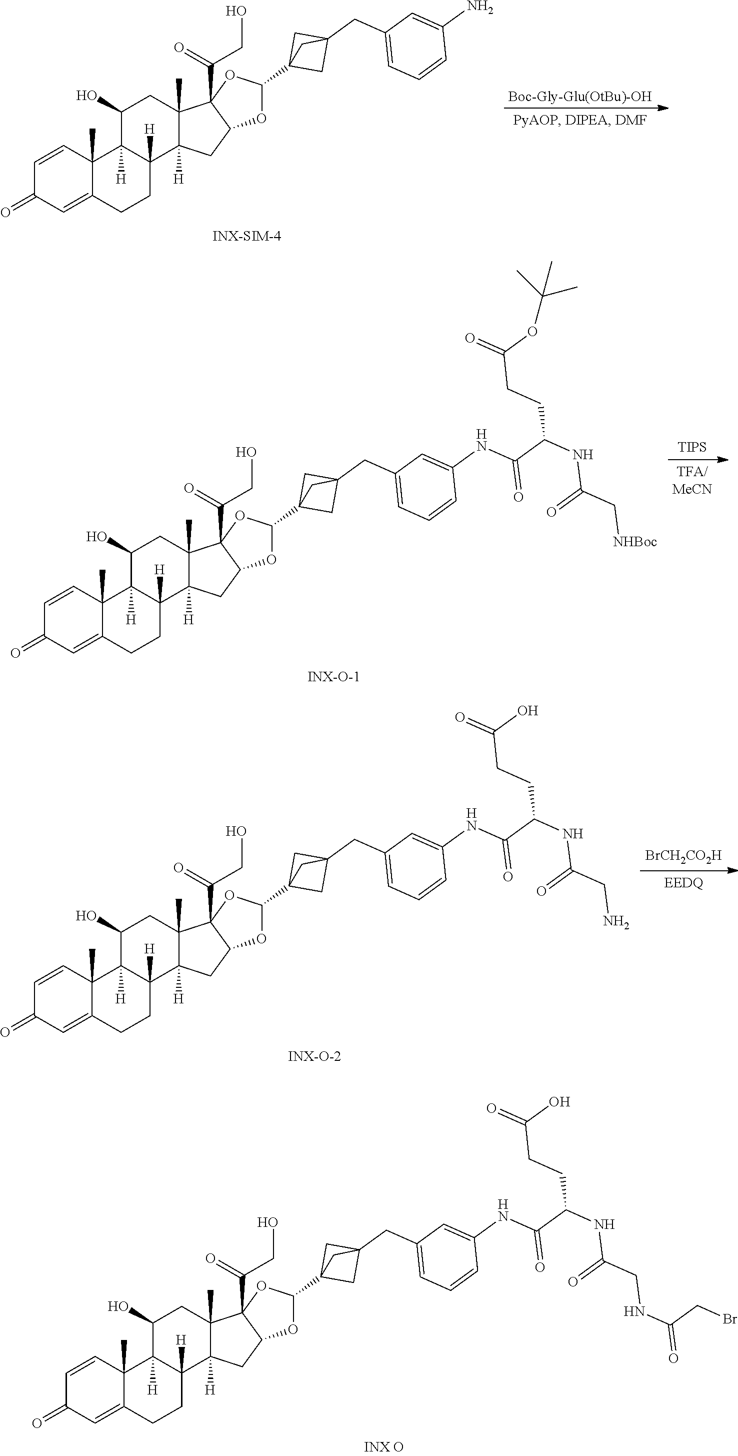

### Synthesis of tert-butyl(S)-4-(2-((tert-butoxycarbonyl)amino)acetamido)-5-((3-((3-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) bicyclo[1.1.1]pentan-1-yl)methyl)phenyl)amino)-5-oxopentanoate (INX O-1)

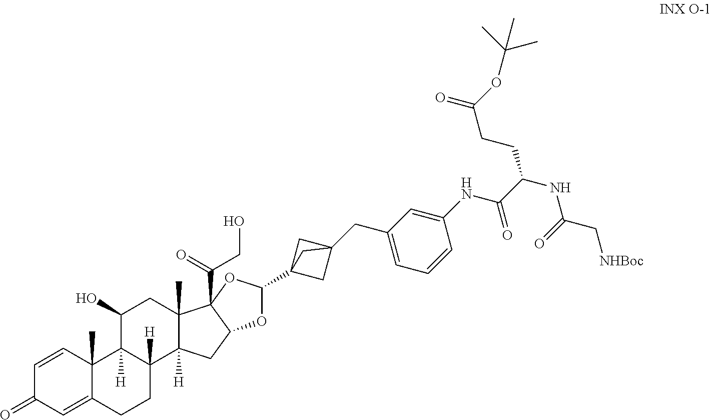

A round bottom flask was charged with INX-SM-4 (0.050 g, 0.0894 mmol, 1.0 eq), Boc-Gly-Glu(OtBu)-OH (0.0805 g, 0.2235 mmol, 2.5 eq), and PyAOP (0.1165 g, 0.2235 mmol, 2.5 eq). DMF (0.10 mL) was added, followed by DIPEA (0.078 mL, 0.4470 mmol, 5.0 eq) and the mixture was stirred for 45 minutes. At this point all INX-SM-4 was consumed and there was a 2:1 ratio of desired product to bis Gly-Glu coupled compound. The crude mixture was loaded onto an Isco C18 Aq 30 g reverse phase column and eluted with a mobile phase of 5-100% acetonitrile (0.05% AcOH additive) in H₂O (0.05% AcOH additive). The fractions containing pure product were combined, frozen, and lyophilized to afford 0.0220 g of INX O-1, 28% yield, as a white solid. LCMS Method B (ESI+): C₅₀H₆₈N₃O₁₂ [M+H]⁺ requires 902.47, found 902.88 at 1.76 minutes.

### Synthesis of (S)-4-(2-aminoacetamido)-5-((3-((3-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) bicyclo[1.1.1]pentan-1-yl)methyl)phenyl)amino)-5-oxopentanoic acid TFA salt (INX O-2)

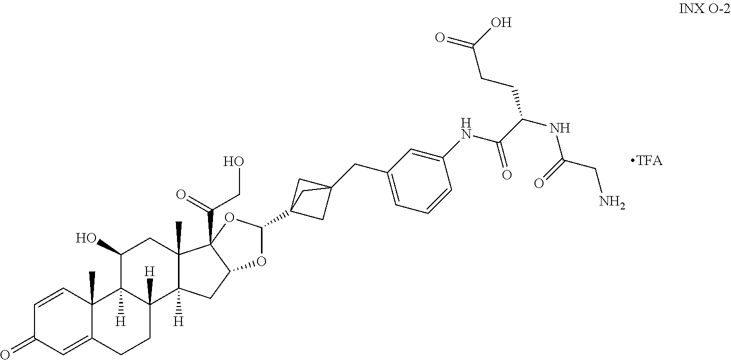

### Procedure

A round bottom flask was charged with tert-butyl ester INX O-1 (0.020 g, 0.022 mmol, 1.0 eq), MeCN (0.50 mL), trifluoroacetic acid (1.0 mL), and triisopropylsilane (0.075 mL, 0.3662 mmol, 16.6 eq). The mixture was allowed to stir for 1 h at room temperature. Starting material consumption was confirmed by LCMS and the solvent was reduced. The resulting residue was loaded onto an Isco C18 Aq 30 g reverse phase column and eluted with a mobile phase of 0-100% acetonitrile (0.10% TFA additive) in H₂O (0.10% TFA additive). The fractions containing pure product were combined, frozen, and lyophilized to afford 0.0144 g of INX O-2.TFA, 76% yield, as a white solid. LCMS Method A (ESI+): C₄₁H₅₂N3010 [M+H]⁺ requires 746.36, found 746.3 at 2.088 minutes.

### Synthesis of (S)-4-(2-(2-bromoacetamido)acetamido)-5-((3-((3-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) bicyclo[1.1.1]pentan-1-yl)methyl)phenyl)amino)-5-oxopentanoic acid (INX Q)

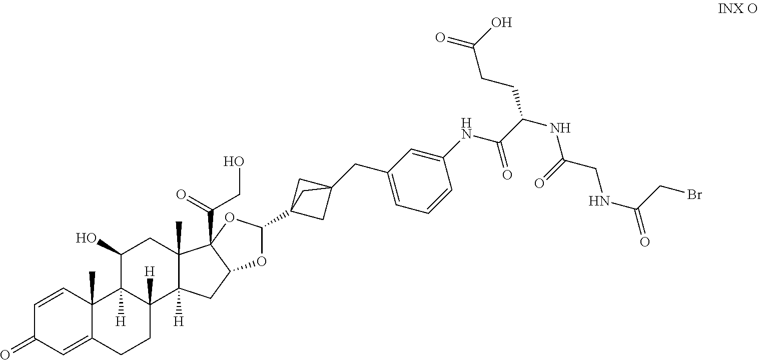

### Procedure

A vial was charged with 2-bromoacetic acid (0.0205 g, 0.1476 mmol, 2 eq), which was dissolved in DMF (0.40 mL). N-Ethoxycarbonyl-2-ethoxy-1,2-dihydroquinoline (0.0347 g, 0.1402 mmol, 2 eq) was added and the mixture was allowed to stir for 90 minutes. Amine INX O-2.TFA (0.0622 g, 0.072 mmol, 1.0 eq) was then added to the solution along with sodium bicarbonate (0.0371 g, 0.4428 mmol, 6.15 eq) and the mixture was allowed to stir for 2 h (until all INX O-2 was consumed). Once reaction completion was confirmed by LCMS, the crude mixture was directly loaded onto an Isco C18 Aq 5.5 g reverse phase column and eluted with a mobile phase of 0-100% acetonitrile (0.05% AcOH additive) in H₂O (0.05% AcOH additive). The fractions containing pure product were combined, frozen, and lyophilized to afford 0.0124 g of INX O, 20% yield, as a fluffy white solid. LCMS Method A (ESI+): C₄₃H₅₃BrN₃O₁₁ [M+H]⁺ requires 866.28, found 866.3 at 2.174 minutes.

### Synthesis of INX-SM-6 and INX-SM-56

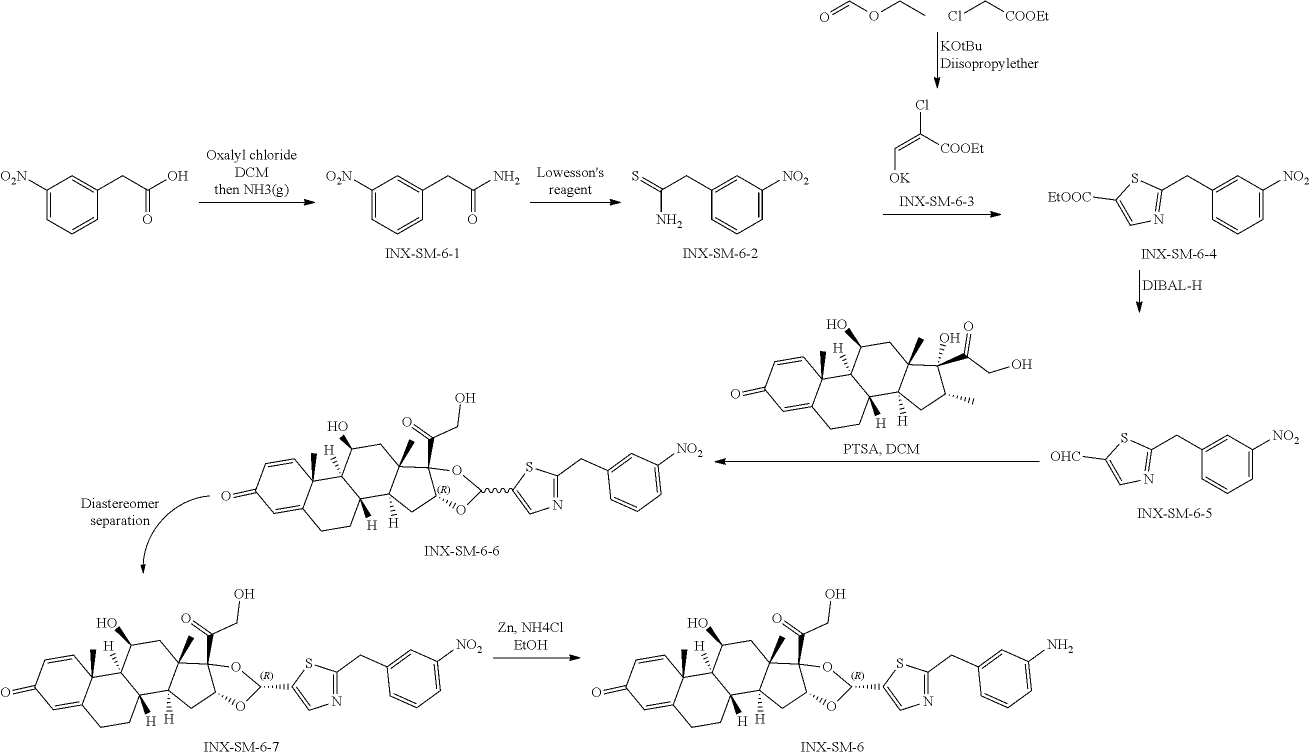

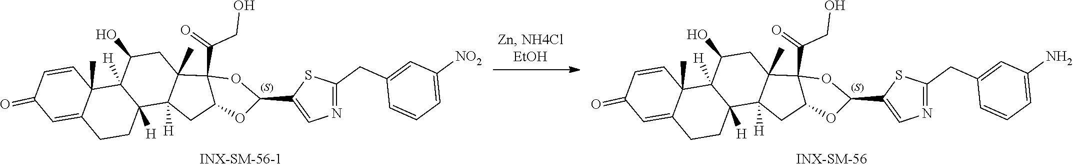

### Synthesis of 2-(3-nitrophenyl) acetamide (INX-SM-6-1)

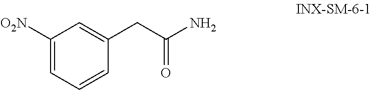 [SMILES: NC(=O)Cc1cccc([N+](=O)[O-])c1]

### Procedure

To a solution of 2-(3-nitrophenyl) acetic acid (0.5 g, 2.76 mmol) in DCM (15 mL), oxalyl chloride (0.71 mL, 8.28 mmol) was added drop wise at 0° C. The reaction mixture was allowed to stir at room temperature for additional 1 h. After completion of reaction as indicated by TLC, reaction mixture was concentrated under vacuum to give gummy liquid which was dissolved in DCM and ammonia gas was purged into at 0° C. After completion of reaction as indicated by TLC, the reaction mixture was quenched with sodium bicarbonate solution and the product was extracted with ethyl acetate. The combined organic layer was dried over Na₂SO₄ and evaporated under vacuum to give title compound as off white solid (0.3 g, 60.33%). The crude was carried forward in next step. LCMS: 181.1 (M+H)⁺.

### Synthesis of 2-(3-nitrophenyl) ethanethioamide (INX-SM-6-2)

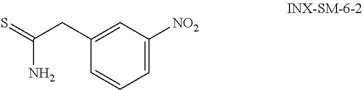 [SMILES: NC(=S)Cc1cccc([N+](=O)[O-])c1]

### Procedure

To a stirred solution of 2-(3-nitrophenyl) acetamide (INX-SM-6-1) (3.0 g, 16.6 mmol) in THF (50 mL), Lawesson's reagent (13.4 g, 33.33 mmol) was added at room temperature and stirred the reaction mixture at reflux temperature for 14 h. After completion of reaction as indicated by TLC, reaction mixture was quenched with water and extracted the product with ethyl acetate. The combined organic layer was dried over Na₂SO₄ and evaporated under vacuum. The crude was purified by silica gel column chromatography (ethyl acetate/hexane: 28:72) to give title compound as pale-yellow solid (3.0 g, 91.76%). LCMS: 197.1 (M+H)⁺; ¹H NMR (DMSO): 9.62, 9.54 (2 brs, 2H), 8.28 (s, 1H), 8.13 (d, J=8.0 Hz, 1H), 7.80 (d, J=7.6 Hz, 1H), 7.63 (t, J=8.0 Hz, 1H), 3.96 (s, 2H).

### Synthesis of potassium 2-chloro-3-ethoxy-3-oxoprop-1-en-1-olate (INX-SM-6-3)

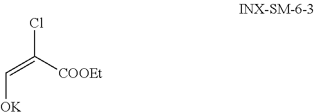

### Procedure

To a solution of methyl ethyl formate (0.5 g, 6.75 mmol) and ethyl 2-chloroacetate (0.824 g, 6.75 mmol) in diisopropyl ether (25 mL), potassium tert-butoxide (0.75 g, 6.75 mmol) was added 0° C. and allowed to stir at rt for 3 h. After completion of reaction as indicated by TLC, reaction mixture was evaporated under vacuum. The crude was purified by triturating with diethyl ether and dried under vacuum to give title compound as yellow solid (0.55 g, 71.40%). ¹H NMR (DMSO-d6) δ: 8.94 (s, 1H), 3.94 (q, 2H), 1.11 (t, 3H).

### Synthesis of ethyl 2-(3-nitrobenzyl) thiazole-5-carboxylate (INX-SM-6-4)

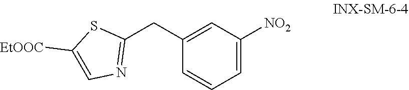 [SMILES: CCOC(=O)c1cnc(Cc2cccc([N+](=O)[O-])c2)s1]

### Procedure

Potassium 2-chloro-3-ethoxy-3-oxoprop-1-en-1-olate (INX-SM-6-3) (5.5 g) was treated with dil. HCl and extracted by ethyl acetate and dried over Na₂SO₄ and concentrated to give yellow semi solid of ethyl 2-chloro-3-oxopropanoate (3.0 g). To a stirred solution of 2-(3-nitrophenyl) ethanethioamide (INX-SM-6-2) (3 g, 15.30 mmol) in ethanol (50 mL), ethyl 2-chloro-3-oxopropanoate (2.75 g, 18.36 mmol) and Na₂SO₄ (8.03 g, 76.53 mmol) was added and stirred at 80° C. for 12 h. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane: 30:70) to give the title compound as yellowish liquid (1.6 g, 35.80%). LCMS: 293.40 (M+H)⁺; ¹H NMR (CDCl3) δ: 8.34 (s, 1H), 8.22-8.19 (m, 2H), 7.70 (d, J=7.6 Hz, 1H), 7.57 (t, J=8 Hz, 1H), 4.49 (s, 2H), 4.32 (q, 2H), 1.31 (t, 3H).

### Synthesis of 2-(3-nitrobenzyl) thiazole-5-carbaldehyde (INX-SM-6-5)

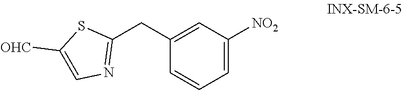 [SMILES: O=Cc1cnc(Cc2cccc([N+](=O)[O-])c2)s1]

### Procedure

To a stirred solution of ethyl 2-(3-nitrobenzyl) thiazole-5-carboxylate (INX-SM-6-4) (1.6 g, 5.4 mmol) in DCM (100 mL), diisobutylaluminum hydride (DIBAL) (1M in toluene, 12.05 ml, 12.05 mmol) was added at −78° C. and stirred further for 20 min at −78° C. After completion of reaction as indicated by TLC, reaction mixture was quenched with dilute HCl solution and allowed to come at room temperature. The product was extracted with DCM. The combined organic layer was washed with brine, dried over Na₂SO₄ and evaporated under vacuum. The crude was purified by silica gel column chromatography (ethyl acetate/hexane: 10:90) to give title compound as off white solid (0.400 g, 29.44%). LCMS: 249.29 (M+H)⁺; ¹H NMR (DMSO-d6) δ: 10.00 (s, 1H), 8.62 (s, 1H), 8.30 (s, 1H), 8.17 (d, J=8.0 Hz, 1H), 7.85 (d, J=7.6 Hz, 1H), 7.67 (t, J=8 Hz, 1H), 4.65 (s, 2H).

### Synthesis of (6aR,6bS,7S,8aS,8bS,10R,11aR,12aS,12bS)-7-hydroxy-8b-(2-hydroxyacetyl)-6a,8a-dimethyl-10-(2-(3-nitrobenzyl) thiazol-5-yl)-1,2,6a,6b,7,8,8a,8b,11a,12,12a,12b-dodecahydro-4H-naphtho[2′,1′:4,5]indeno[1,2-d][1,3]dioxol-4-one (INX-SM-6-7); and (6aR,6bS,7S,8aS,8bS,10S,11aR,12aS,12bS)-7-hydroxy-8b-(2-hydroxyacetyl)-6a,8a-dimethyl-10-(2-(3-nitrobenzyl) thiazol-5-yl)-1,2,6a,6b,7,8,8a,8b,11a,12,12a,12b-dodecahydro-4H-naphtho[2′,1′:4,5]indeno[1,2-d][1,3]dioxol-4-one (INX-SM-56-1)

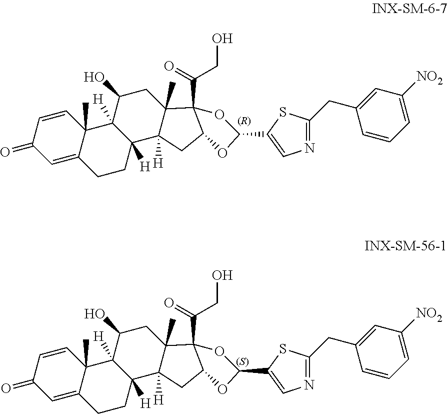

### Procedure

To a stirred solution of 2-(3-nitrobenzyl) thiazole-5-carbaldehyde ((INX-SM-6-5) (0.4 g, 1.06 mmol) in DCM (20 mL) was added (8S,9S,10R,11S,13S,14S,16R,17S)-11,16,17-trihydroxy-17-(2-hydroxyacetyl)-10, 13-dimethyl-6, 7,8,9,10,11,12,13,14,15,16,17-dodecahydro-3H-cyclopenta[a]phenanthren-3-one (16-alpha-hydroxyprednisolone) (0.211 g, 0.84 mmol) and p-toluenesulfonic acid (1.0 g, 5.30 mmol) and stirred for 8 h at room temperature. After completion of reaction as indicated by TLC, reaction mixture was quenched with bicarbonate solution and extracted with DCM. The combined organic layer was dried over Na₂SO₄ and evaporated under vacuum. The crude was purified by flash chromatography (Methanol/DCM: 6:94) to give compound as mixture of diastereomers (INX-SM-6-6).

Further the diastereomers were separated by prep HPLC (Column: YMC-Actus Triart Prep C18-S,250×20 mm S-10 μm, 12 mm, Mobile phase: A=0.05% ammonia in water, B=20% A-Line in ACN, A:B=45:55). These isomers were eluted at retention time 13.5 min (INX-SM-6-7, Isomer-1) (0.030 g, 8.8%) and 18.50 min (INX-SM-56-1, Isomer-2) (0.040 g, 11.8%).

### Synthesis of ((6aR,6bS,7S,8aS,8bS,10R,11aR,12aS,12bS)-10-(2-(3-aminobenzyl) thiazol-5-yl)-7-hydroxy-8b-(2-hydroxyacetyl)-6a,8a-dimethyl-1, 2, 6a,6b,7,8,8a,8b, 11a,12,12a,12b-dodecahydro-4H-naphtho[2′,1′:4,5]indeno[1,2-d][1,3]dioxol-4-one (INX-SM-6)

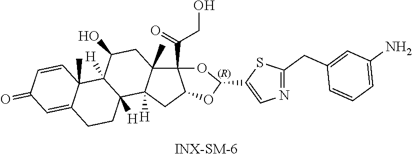

### Procedure

To a stirred solution of (INX-SM-6-7, Isomer-1) (0.030 g, 0.049 mmol) in ethanol (2 mL) was added NH₄Cl (0.020 g, 0.39 mmol) and Zn metal (0.025 g, 0.39 mmol). The reaction mixture was heated at 80° C. for 2 h. After completion of reaction as indicated by TLC, reaction mixture was filtered, and filtrate was evaporated under vacuum. The crude was purified by reverse phase prep HPLC (0.05% Ammonia-Acetonitrile) to give title compound as white solid (0.005 g, 17.8%).
INX-SM-6 (R-Isomer): LCMS: 577.2 (M+H)⁺; ¹H NMR (400 MHZ, MeOD, Key proton assignment): δ: 5.86 (s, 1H, Acetal-H), 5.02 (d, C-16H).

### Synthesis of (6aR,6bS,7S,8aS,8bS,10S,11aR,12aS,12bS)-10-(2-(3-aminobenzyl) thiazol-5-yl)-7-hydroxy-8b-(2-hydroxyacetyl)-6a,8a-dimethyl-1, 2, 6a,6b,7,8,8a,8b, 11a,12,12a,12b-dodecahydro-4H-naphtho[2′,1′:4,5]indeno[1,2-d][1,3]dioxol-4-one (INX-SM-56)

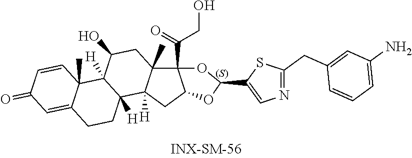

### Procedure

To a stirred solution of (INX-SM-56-1, Isomer-2) (0.040 g, 0.065 mmol) in ethanol (2 mL) was added NH₄Cl (0.027 g, 0.52 mmol) and Zn metal (0.034 g, 0.52 mmol). The reaction mixture was heated at 80° C. for 2 h. After completion of reaction as indicated by TLC, reaction mixture was filtered, and filtrate was evaporated under vacuum. The crude was further purified by reverse phase prep HPLC (0.05% Ammonia-Acetonitrile) to give title compound as white solid (0.015 g, 39%); LCMS: 577.1 (M+H)⁺.
INX-SM-56 (S-Isomer): LCMS: 577.2 (M+H)⁺; ¹H NMR (400 MHZ, MeOD, Key proton assignment): δ: 6.40 (s, 1H, Acetal-H), 5.33 (d, J=6.0 Hz, C-16H)

### Synthesis of INX-SM-7 & INX-SM-57

### Reaction Scheme

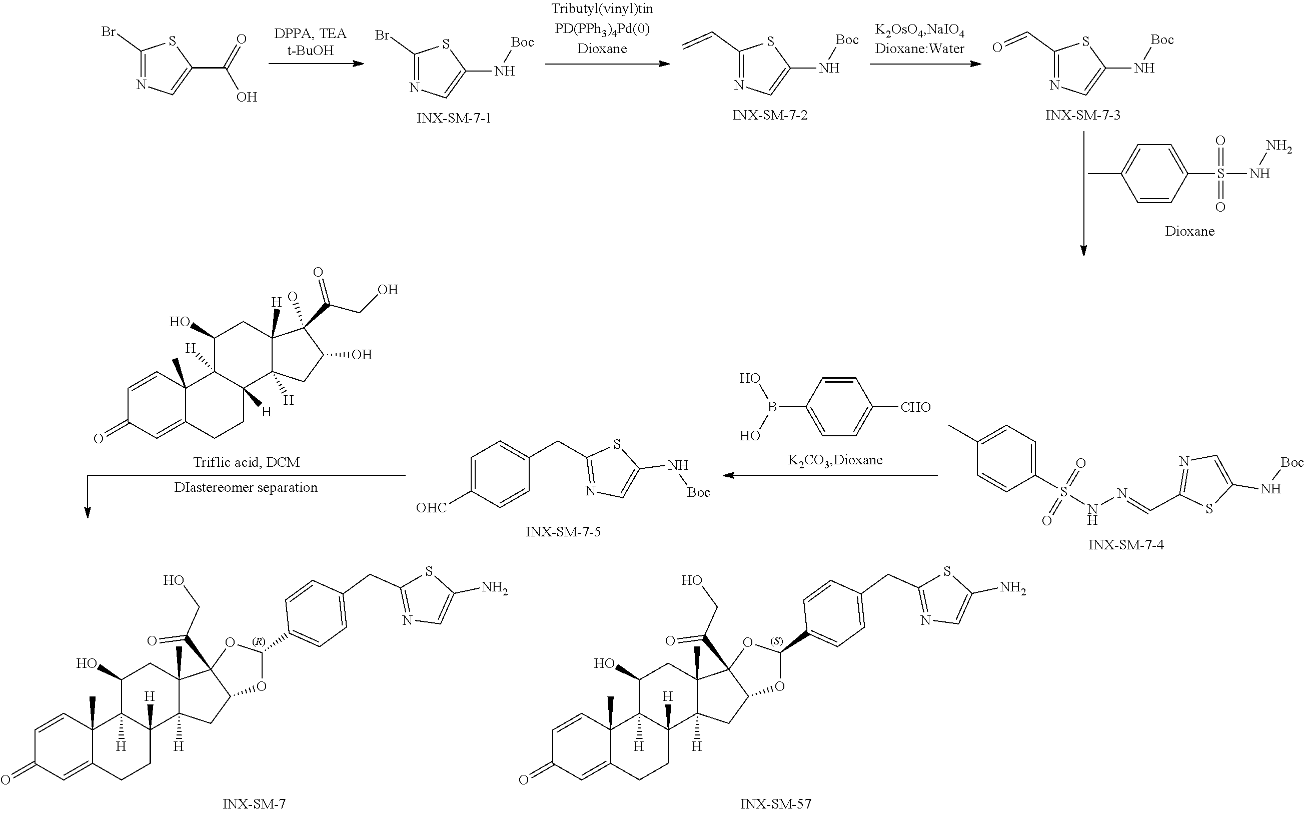

### Synthesis of tert-butyl (2-bromothiazol-5-yl)carbamate (INX-SM-7-1)

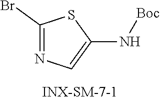 [SMILES: CC(C)(C)OC(=O)Nc1cnc(Br)s1]

### Procedure

To a solution of 2-bromothiazole-5-carboxylic acid (5.0 g, 24.0 mmol) in t-BuOH (50 mL), diphenylphosphoryl azide (DPPA) (7.74 mL, 36.0 mmol) and triethylamine (13.48 ml, 96.1 mmol) were added and allowed to stir at 80° C. for 12 h. After completion of reaction as indicated by TLC, reaction mixture was evaporated under vacuum. The crude was purified by silica gel column chromatography (ethyl acetate/hexane: 10:90) to give title compound as brown solid (2.3 g, 34.28%). LCMS: 278 (M+H)⁺; ¹H NMR (DMSO-d6): 10.98 (s, 1H), 7.09 (s, 1H) 1.46 (s, 9H).

### Synthesis of tert-butyl (2-vinylthiazol-5-yl)carbamate (INX-SM-7-2)

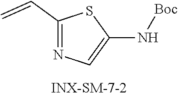

### Procedure

To a stirred solution of tert-butyl (2-bromothiazol-5-yl)carbamate (INX-SM-7-1) (1.5 g, 5.37 mmol) dioxane (50 mL), tributyl (vinyl) tin (1.70 g, 5.37 mmol) was added at room temperature and degassed with N₂ (g) for 15 min. Tetrakis triphenylphosphine palladium (0) (0.310 g, 0.26 mmol) was added to the reaction mixture and stirred the reaction mixture at 100° C. for 12 h. After completion of reaction as indicated by TLC, reaction mixture was filtered through celite and filtrate was evaporated under vacuum. The crude was purified by silica gel column chromatography (ethyl acetate/hexane: 20:80) to give title compound (0.9 g, 74.2%). LCMS: 227.0 (M+H)⁺; ¹H NMR (DMSO-d6): 10.72 (s, 1H), 7.26 (s, 1H), 6.76 (dd, J=11.2 & 17.6 Hz, 1H), 5.82 (d, J=17.6 Hz, 1H), 5.42 (d, J=11.2 Hz, 1H), 1.46 (s, 9H).

### Synthesis of tert-butyl (2-formylthiazol-5-yl)carbamate (INX-SM-7-3)

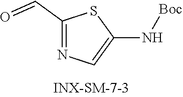 [SMILES: CC(C)(C)OC(=O)Nc1cnc(C=O)s1]

### Procedure

To a solution of tert-butyl (2-vinylthiazol-5-yl)carbamate (INX-SM-7-2) (3.8 g, 16.8 mmol) in dioxane (50 mL), a solution of K₂OsO₄·2H₂O (0.179 g, 0.48 mmol) in water (2 ml) was added. NaIO₄ (18.15 g, 85.2 mmol) was dissolved in water (10 ml) and added to the reaction mixture stirred at rt for 3 h. After completion of reaction as indicated by TLC, reaction mixture was filtered through celite bad and filtrate was evaporated under vacuum. The crude was purified by silica gel column chromatography (ethyl acetate:hexane: 15:85) to give the title compound as a pale-yellow solid (2.5 g, 65.22%). LCMS: 229.0 (M+H)⁺.

### Synthesis of tert-butyl-(2-((2-tosylhydrazono)methyl)thiazol-5-yl)carbamate (INX-SM-7-4)

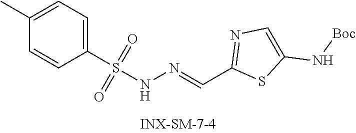 [SMILES: Cc1ccc(S(=O)(=O)N/N=C/c2ncc(NC(=O)OC(C)(C)C)s2)cc1]

### Procedure

To a solution of tert-butyl (2-formylthiazol-5-yl)carbamate (INX-SM-7-3) (2.5 g, 10.9 mmol) in dioxane (50 mL), p-toluenesulphonylhydrazide (2.23 g, 12.0 mmol) was added and stirred the reaction mixture at 90° C. for 5 h. After completion of reaction as indicated by TLC, reaction mixture was evaporated under vacuum. The crude was purified by silica gel column chromatography (ethyl acetate:hexane: 25:75) to give title compound as a pale-yellow solid (2.8 g, 64.48%). LCMS: 397.0 (M+H)⁺.

### Synthesis of tert-butyl (2-(4-formylbenzyl) thiazol-5-yl)carbamate (INX-SM-7-5)

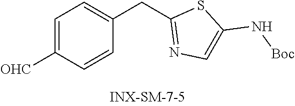 [SMILES: CC(C)(C)OC(=O)Nc1cnc(Cc2ccc(C=O)cc2)s1]

### Procedure

To a stirred solution of tert-butyl-(2-((2-tosylhydrazono)methyl)thiazol-5-yl) carbamate (INX-SM-7-4) (2.8 g, 7.06 mmol) in dioxane (50 mL), (4-formylphenyl)boronic acid (1.16 g, 7.76 mmol) and K₂CO3 (1.94 g, 14.12 mmol) were added and stirred at 110° C. for 2 h. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane: 20:80) to give title compound as a pale-yellow solid (0.4 g, 17.79%). LCMS: 319.0 (M+H)⁺.

### Synthesis of (6aR,6bS,7S,8aS,8bS,10R,11aR,12aS,12bS)-10-(4-((5-aminothiazol-2-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-7); and

### (6aR,6bS,7S,8aS,8bS,10S,11aR,12aS,12bS)-10-(4-((5-aminothiazol-2-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-57)

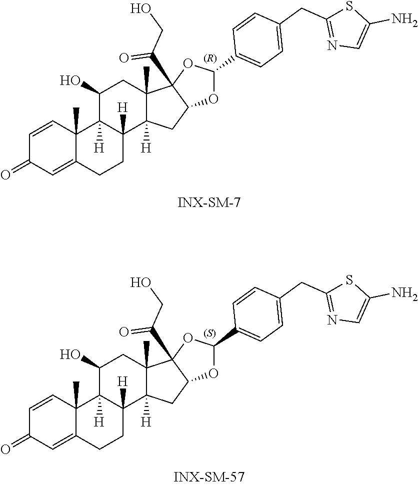

### Procedure

To a stirred solution of tert-butyl (2-(4-formylbenzyl) thiazol-5-yl)carbamate (INX-SM-7-5) (0.1 g, 0.31 mmol) and (8S,9S,10R,11S,13S,14S,16R,17S)-11,16,17-trihydroxy-17-(2-hydroxyacetyl)-10, 13-dimethyl-6, 7, 8, 9, 10, 11, 12, 13, 14, 15, 16, 17-dodecahydro-3H-cyclopenta[a]phenanthren-3-one (16-alpha-hydroxyprednisolone) (0.118 g, 0.31 mmol) in DCM (50 mL), a solution of triflic acid (0.15 g, 1.03 mmol) in acetonitrile (6.2 ml) was added and stirred at room temperature for 1 h. After completion of reaction as indicated by TLC, reaction mixture was poured into saturated NaOH Solution and extracted with MDC. The combined organic layer was dried over Na₂SO₄ and evaporated under vacuum to give title compound as mixture of isomer (0.060 g, crude).

Further the diastereomers were separated by prep HPLC (Column: Xbridge prep, C18, OBD19*250 mm, 5 micron, Mobile phase: A=0.05% ammonia in water, B=ACN (67:33), A:B=67:33) to give Isomer-1 and Isomer-2. These isomers were eluted at retention time 17.70 min (Isomer-1) and 20.87 min (Isomer-2).
INX-SM-7 (Isomer-1, R-Isomer): (Yield: 0.010 g, 3%). LCMS: 577.4 (M+H)⁺; ¹H NMR (400 MHz, MeOD, Key proton assignment): δ: 5.47 (s, 1H, Acetal-H), 5.06 (d, J=5.2 Hz, 1H, C16H)
INX-SM-57 (Isomer-2, S Isomer): (Yield: 0.003 g, 0.6%). LCMS 577.4 (M+H)⁺; ¹H NMR (400 MHZ, MeOD, Key proton assignment): δ: 6.03 (s, 1H, Acetal-H), 5.41 (d, J=5.2 Hz, 1H, C16H)

### Synthesis of INX-SM-13 and INX-SM-63

### Reaction Scheme

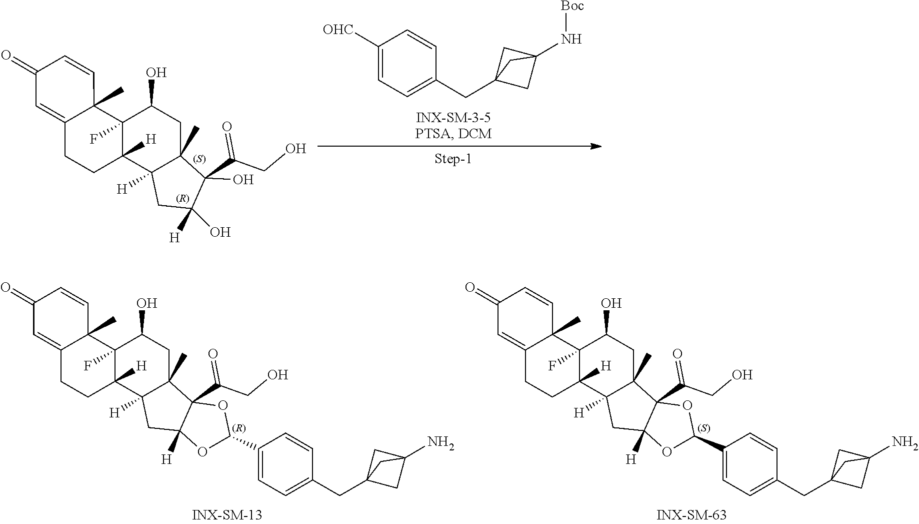

### Synthesis of (6aS,6bR,7S,8aS,8bS,10R,11aR,12aS,12bS)-10-(4-((3-aminobicyclo[1.1.1]pentan-1-yl)methyl)phenyl)-6b-fluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-13); and

### (6aS,6bR,7S,8aS,8bS,10S,11aR,12aS,12bS)-10-(4-((3-aminobicyclo[1.1.1]pentan-1-yl)methyl)phenyl)-6b-fluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-63)

### Procedure

To a solution of tert-butyl (3-(4-formylbenzyl)bicyclo[1.1.1]pentan-1-yl)carbamate (INX-SM-3-5) (0.180 g, 0.597 mmol) and (8S,9R,10S,11S,13S,14S,16R,17S)-9-fluoro-11, 16, 17-trihydroxy-17-(2-hydroxyacetyl)-10, 13-dimethyl-6,7,8,9, 10, 11, 12, 13, 14, 15, 16, 17-dodecahydro-3H-cyclopenta[a]phenanthren-3-one (Triamcinolone) (0.259 g, 0.656 mmol) in DCM (2 mL), p-toluenesulfonic acid (0.908 g, 4.77 mmol) was added and stirred at room temperature for another 16 h. After completion of reaction as indicated by TLC, reaction mixture was poured into sat. NaHCO₃ solution and extracted with ethyl acetate. The combined organic layer was dried over Na₂SO₄ and evaporated under vacuum to give crude product compound as mixture of isomers. Further the crude product was purified and isomers were separated by reverse phase prep-HPLC (Column: YMC-Actus Triart Prep C18-S, 250×20 mm S-10 μm, 12 nm, Mobile phase: A=0.05% Ammonia in Water, B=ACN: MeOH (50:50). These isomers were eluted at retention time 14 min (Isomer-1) and 19.5 min (Isomer-2).
INX-SM-13 (Isomer-1): (Yield: 0.038 g, 11.08%). LCMS: 578.20 (M+H)⁺; ¹H NMR (400 MHZ, MeOD, Key proton assignment): δ: 5.47 (s, 1H, Acetal-H), 5.05 (d, J=5.2 Hz, 1H, C16H)
INX-SM-63 (Isomer-2): (Yield: 0.005 g, 1.45%). LCMS 578.30 (M+H)⁺; ¹H NMR (400 MHZ, MeOD, Key proton assignment): δ: 6.13 (s, 1H, Acetal-H), 5.42 (d, J=6.8 Hz, 1H, C16H)

### Synthesis of INX-SM-24 and INX-SM-74

### Reaction Scheme

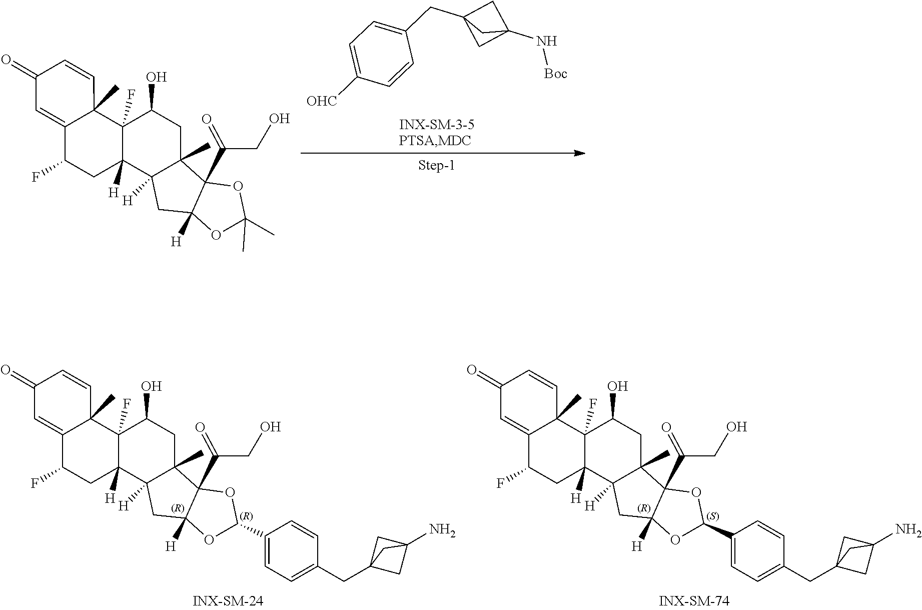

### Synthesis of (2S,6aS,6bR,7S,8aS,8bS,10R,11aR,12aS,12bS)-10-(4-((3-aminobicyclo[1.1.1]pentan-1-yl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-24); and (2S,6aS,6bR,7S,8aS,8bS,10S,11aR,12aS,12bS)-10-(4-((3-aminobicyclo[1.1.1]pentan-1-yl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-74)

### Procedure

To a solution of tert-butyl (3-(4-formylbenzyl)bicyclo[1.1.1]pentan-1-yl)carbamate (INX-SM-3-5) (0.500 g, 1.66 mmol) and (2S,6aS,6bR,7S,8aS,8bS,11aR,12aS,12bS)-2,6b-difluoro-7-hydroxy-8b-(2-hydroxyacetyl)-6a,8a,10, 10-tetramethyl-1,2,6a,6b,7,8,8a,8b,11a,12,12a,12b-dodecahydro-4H-naphtho[2′,1′:4,5]indeno[1,2-d][1,3]dioxol-4-one (Fluocinolone acetonide) (0.716 g, 1.65 mmol) in DCM (10 mL), p-toluenesulfonic acid (2.5 g, 13.26 mmol) was added and stirred at room temperature for another 16 h. After completion of reaction as indicated by TLC, reaction mixture was poured into sat. NaHCO₃ solution and extracted with ethyl acetate. The combined organic layer was dried over Na₂SO₄ and evaporated under vacuum to give the crude product as mixture of isomers. Further the crude product was purified and isomers were separated by reverse phase prep-HPLC (Column: Unisil 10-120 C18 Ultra, 250×21.2 mm×10 μm, Mobile phase: A=0.05% Ammonia in Water, B=Acetonitrile) to give Isomer-1 and Isomer-2. These isomers were eluted at retention time 13.5 min. (Isomer-1) and 19.5 min (Isomer-2).
INX-SM-24 (R-Isomer): (Yield 0.100 g, 10.11%).). LCMS: 596.20 (M+H)⁺; ¹H NMR (400 MHZ, MeOD, Key proton assignment): δ: 5.48 (s, 1H, Acetal-H), 5.06 (d, J=4.4 Hz, 1H, C16H)
INX-SM-74 (S-isomer): (Yield 0.020 g, 2.02%). LCMS: 596.20 (M+H)⁺; ¹H NMR (400 MHZ, MeOD, Key proton assignment): δ: 6.17 (s, 1H, Acetal-H), 5.43 (d, J=7.2 Hz, 1H, C16H)

### Synthesis of (6aR,6bS,7S,8aS,8bS,10R,11aR,12aS,12bS)-10-(4-((4-aminocuban-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-9)

### Reaction Scheme

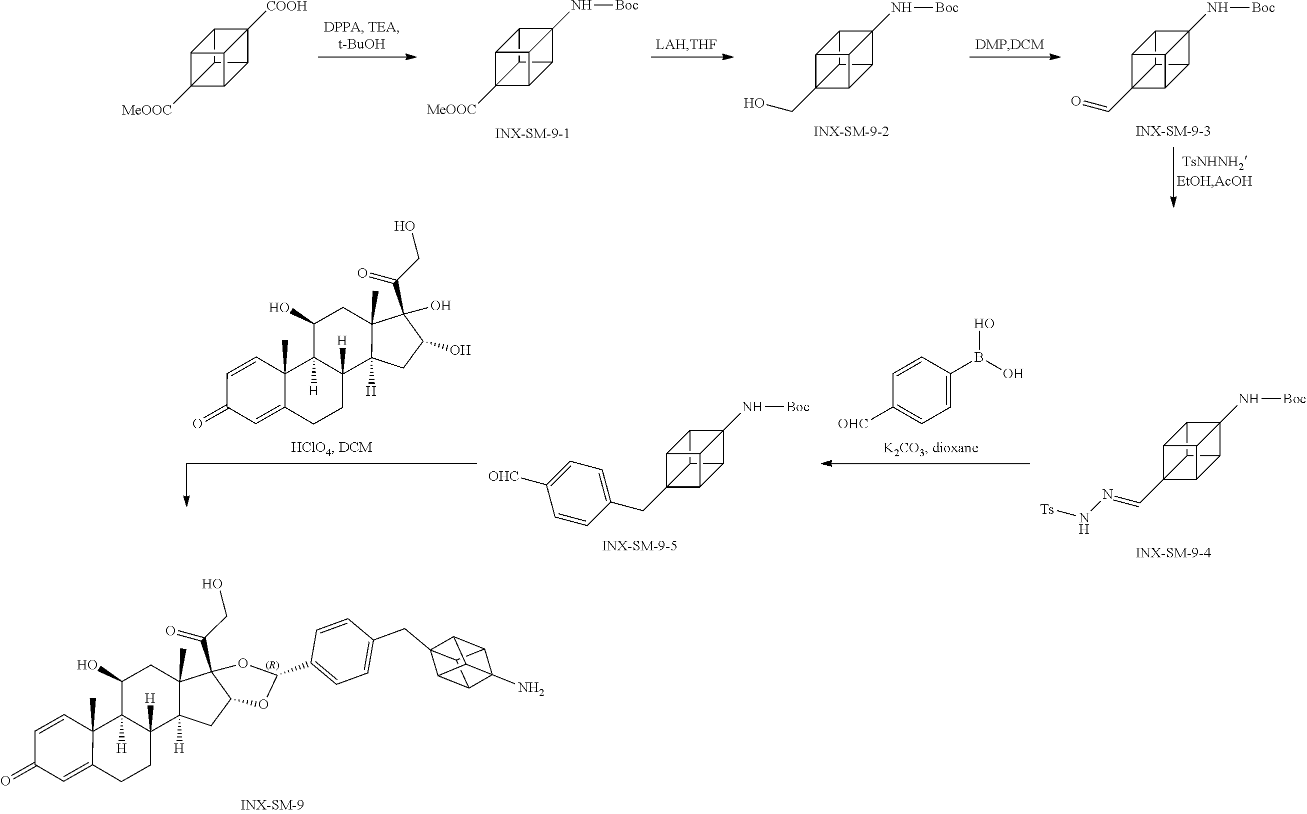

### Synthesis of methyl 4-((tert-butoxycarbonyl)amino)cubane-1-carboxylate (INX-SM-9-1)

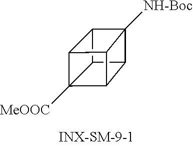

### Procedure

A 100 mL single-necked round bottom flask was charged with 4-methoxycarbonyl cubanecarboxylic acid (2 g, 9.69 mmol) and tert-Butyl alcohol (60 mL). To this solution, diphenylphosphoryl azide (DPPA) (3.1 mL, 14.54 mmol) and triethylamine (10.8 mL, 77.59 mmol) were added at room temperature and stirred for 30 min at room temperature. The reaction mixture was heated at 80° C. for 1 h. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane: 15:85) to give title compound as white solid (0.90 g, 33.46%). ¹H NMR (CDCl₃) δ: 4.1 (bs, 6H), 3.71 (s, 3H), 1.46 (s, 9H).

### Synthesis of tert-butyl (4-(hydroxymethyl)cuban-1-yl)carbamate (INX-SM-9-2)

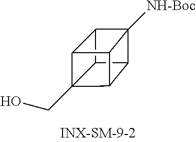

A 100 mL three-neck round bottom flask was charged with methyl 4-((tert-butoxycarbonyl)amino) cubane-1-carboxylate (INX-SM-9-1) (0.9 g, 3.24 mmol) and THF (40 mL) under nitrogen. To this solution, 1M lithium aluminium hydride in THF (3.2 mL, 3.24 mmol) was added at −78° C. and stirred for another 1 h. After completion of reaction as indicated by TLC, reaction mixture was quenched with 1N NaOH solution and extracted with ethyl acetate. The combined organic layer was dried over Na₂SO₄ and evaporated under vacuum to give crude product (0.8 g, 98.88%). ¹H NMR (DMSO-d6) δ: 7.58 (bs, 1H), 4.42 (t, 1H), 3.80 (bs, 3H), 3.57 (bs, 3H) 3.48 (d, 2H, J=5.2), 1.37 (s, 9H).

### Synthesis of 4tert-butyl (4-formylcuban-1-yl)carbamate (INX-SM-9-3)

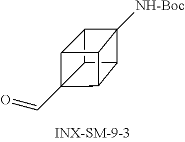

### Procedure

A 100 mL three-necked round bottom flask was charged with tert-butyl (4-(hydroxymethyl) cuban-1-yl)carbamate (INX-SM-9-2) (0.9 g, 3.60 mmol) and DCM (25 mL) under nitrogen. To this solution, Dess-Martin periodinane (DMP) (3.06 g, 7.21 mmol) was added at 0° C. and stirred for 1 h. After completion of reaction as indicated by TLC, reaction mixture was filtered through celite and washed with diethyl ether. The combined filtrate was evaporated under vacuum to give title compound as white solid (1.0 g, crude, quantitative). The crude was used immediately for next step.

### Synthesis tert-butyl (4-((2-tosylhydrazono)methyl)cuban-1-yl)carbamate (INX-SM-9-4)

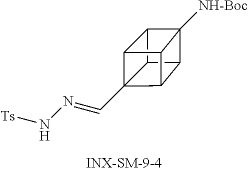

### Procedure

A 50 mL single-necked round bottom flask was charged with tert-butyl (4-formylcuban-1-yl)carbamate (INX-SM-9-3) (1.0 g, 4.04 mmol) and EtOH (30 mL) under nitrogen. To this solution, p-toluenesulfonylhydrazide (1.1 g, 6.06 mmol) was added with catalytic amount of AcOH and stirred for 1 h at room temperature. After completion of reaction as indicated by TLC, reaction mixture was poured into water. The solid was filtered and the product was dried under vacuum. The crude was purified by silica gel column chromatography (ethyl acetate:hexane, 1:4) to give title compound as white solid (0.8 g, 47.61%). LCMS: 416.3 (M+H)⁺; ¹H NMR (DMSO-d6) δ: 11.07 (s, 1H), 7.67 (d, J=8.4 Hz, 2H), 7.40-7.38 (m, 3H), 3.85-3.81 (m, 6H), 2.38 (s, 3H), 1.36 (s, 9H).

### Synthesis of tert-butyl (4-(4-formylbenzyl)cuban-1-yl)carbamate (INX-SM-9-5)

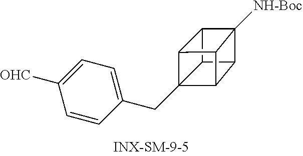

A 35 mL vial was charged with tert-butyl (4-((2-tosylhydrazono)methyl)cuban-1-yl)carbamate (INX-SM-9-4) (0.50 g, 1.20 mmol) and dioxane (10 mL) under nitrogen. The reaction mixture was purged for 10 min with N2. To this solution, (4-formyl phenyl)boronic acid (0.36 g, 2.40 mmol) and K₂CO3 (0.33 g, 2.41 mmol) were added at room temperature and stirred for 1 h at 110° C. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane, 15:85) to give title compound as white solid (0.040 g, 9.85%). ¹H NMR (DMSO-d6) δ: 9.96 (s, 1H), 7.83 (d, J=8 Hz, 2H), 7.59 (bs, 1H), 7.40 (d, J=7.6 Hz, 2H), 3.76 (bs, 3H), 3.58 (bs, 3H), 2.96 (s, 2H), 1.35 (s, 9H).

### Synthesis of (6aR,6bS,7S,8aS,8bS,10R,11aR,12aS,12bS)-10-(4-((4-aminocuban-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-9)

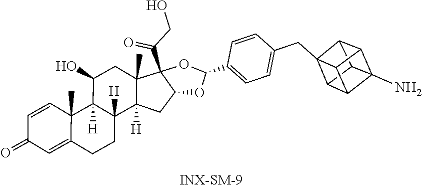

### Procedure

A 10 mL single-necked round bottom flask was charged with tert-butyl ((2r,3R,4s,5S)-4-(4-formylbenzyl)cuban-1-yl)carbamate (INX-SM-9-5) (0.035 g, 0.10 mmol) and (8S,9S,10R,11S,13S,14S,16R,17S)-11,16, 17-trihydroxy-17-(2-hydroxyacetyl)-10,13-dimethyl-6,7,8,9,10,11, 12,13,14,15,16,17-dodecahydro-3H-cyclopenta[a]phenanthren-3-one (16-alpha-hydroxy prednisolone) (0.038 g, 0.10 mmol), MgSO₄ (0.062 g, 0.51 mmol) and DCM (10 mL). To this solution, HClO₄ (0.157 g, 1.55 mmol) was added and stirred for 1 h at room temperature. After completion of reaction as indicated by TLC, reaction mixture was quenched with sat. NaHCO₃ solution and concentrated under vacuum. The crude was triturate with cold water and participated was filtered and dried under vacuum. The crude was purified by prep-HPLC (Column: SUNFIRE Prep C18 OBD, 19×250 mm, 5 μm, Mobile phase: A=0.1% FA in Water, B=ACN: MeOH: IPA (65:25:10), A:B, 67:33); Retention time 15.14 min to give R-Isomer as white solid (0.010 g, 16.18%); LCMS: 597.4 (M+H)⁺; ¹H NMR (400 MHZ, MeOD, Key proton assignment): δ: 5.47 (s, 1H, Acetal-H), 5.06 (d, J=4.8 Hz, 1H, C16H).

### Synthesis of (6aR,6bS,7S,8aS,8bS,11aR,12aS,12bS)-10-(4-((6-aminospiro[3.3]heptan-2-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-32)

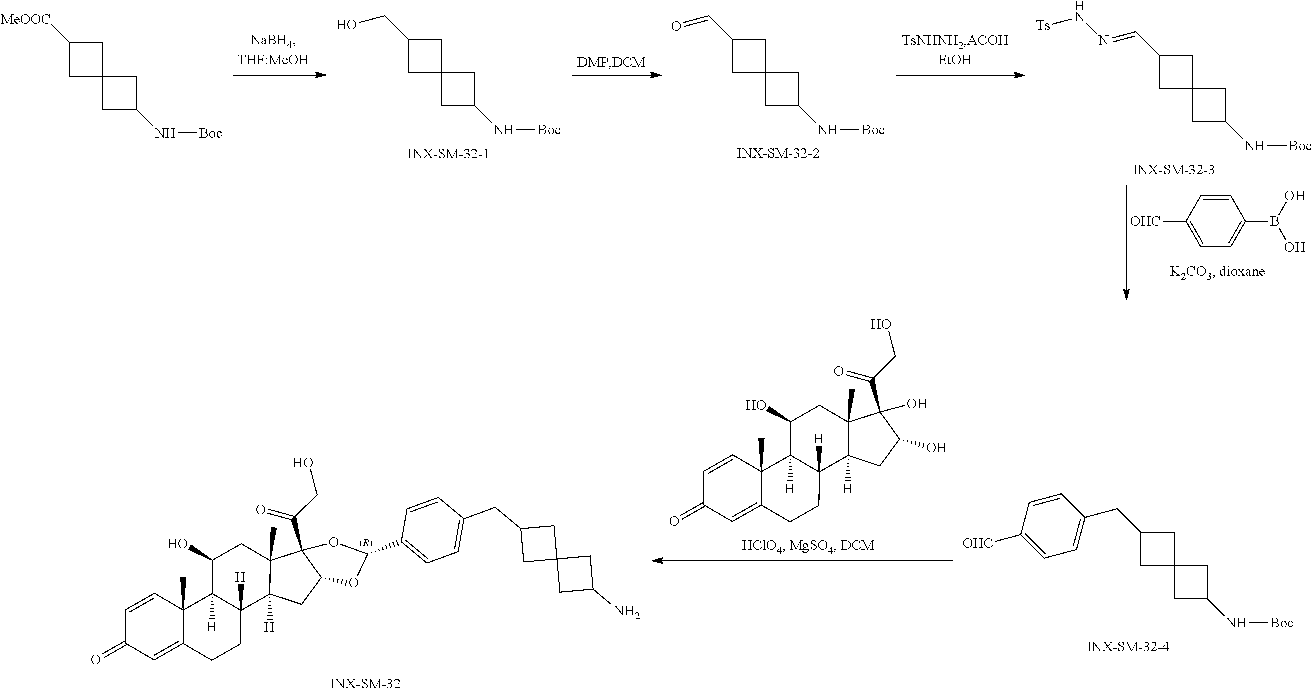

### Synthesis of tert-butyl (6-(hydroxymethyl)spiro[3.3]heptan-2-yl)carbamate (INX-SM-32-1)

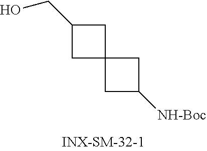

### Procedure

A 50 mL single-necked round bottom flask was charged with methyl 6-((tert-butoxycarbonyl)amino) spiro[3.3]heptane-2-carboxylate (2.0 g, 7.43 mmol) and THF:MeOH (15:5 mL) under nitrogen. To this solution, NaBH₄ (1.4 g, 37.17 mmol) was added portion-wise at 0° C. and stirred for another 4 h at room temperature. After completion of reaction as indicated by TLC, reaction mixture was dilute with water and adjusted neutral pH with 1N HCl. The product was extracted with ethyl acetate. The combined organic layer was dried over Na₂SO₄ and evaporated under vacuum to give crude product (2.0 g, quantitative). LCMS: 186.2 (M+H−56), ¹H NMR (CDCl3) δ: 4.63 (bs, 1H), 3.97 (bs, 1H), 3.54 (d, J=6.8 Hz, 2H), 2.50-2.25 (m, 3H), 2.20-1.95 (m, 2H), 1.90-1.40 (m, 5H), 1.46 (s, 9H).

### Synthesis of tert-butyl (6-formylspiro[3.3]heptan-2-yl)carbamate (INX-SM-32-2)

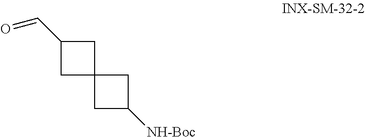

### Procedure

A 50 mL single-necked round bottom flask was charged with tert-butyl (6-(hydroxymethyl)spiro[3.3]heptan-2-yl)carbamate (INX-SM-32-1) (2.0 g, 8.30 mmol) and DCM (20 mL) under nitrogen. To this solution, Dess-Martin periodinane (DMP) (3.51 g, 8.30 mmol) was added at 0° C. and stirred for 2 h. After completion of reaction as indicated by TLC, reaction mixture was filtered through celite and washed with diethyl ether. The combined organic layer was evaporated under vacuum. The crude was purified by silica gel column chromatography (ethyl acetate/hexane, 40:60) to give title compound as yellow solid (1.7 g, 85.72%). LCMS: 184.2 (M+H-56).

### Synthesis of tert-butyl (6-((2-tosylhydrazono)methyl)spiro[3.3]heptan-2-yl)carbamate (INX-SM-32-3)

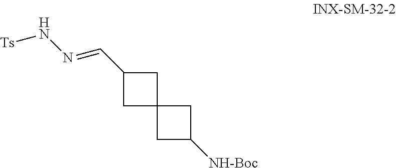

### Procedure

A 50 mL single-necked round bottom flask was charged with tert-butyl (6-formylspiro[3.3]heptan-2-yl)carbamate (INX-SM-32-2) (1.5 g, 6.27 mmol) and EtOH (15 mL) under nitrogen. To this solution, p-toluenesulfonhydrazide (1.16 g, 6.27 mmol) and catalytic amount of AcOH (0.2 mL) were added and stirred for 2 h at room temperature. After completion of reaction as indicated by TLC, reaction mixture was poured into water. The solid was filtered and dried under vacuum to give title compound as white solid (2.2 g, 86.13%). LCMS: 425.5 (M+18).

### Synthesis of tert-butyl (6-(4-formylbenzyl) spiro[3.3]heptan-2-yl)carbamate (INX-SM-32-4)

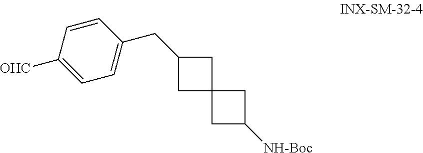

### Procedure

A 35 mL vial was charged with tert-butyl (6-((2-tosylhydrazono)methyl) spiro[3.3]heptan-2-yl)carbamate (INX-SM-32-3) (1.0 g, 2.45 mmol) and dioxane (10 mL) under nitrogen. To this solution, (4-formylphenyl)boronic acid (0.36 g, 2.45 mmol) and K₂CO3 (0.51 g, 3.68 mmol) were added at room temperature and stirred at 100° C. for another 2 h. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane, 30:70) to give title compound as yellow solid (0.16 g, 19.79%). LCMS: 274.3 (M+H-56).

### Synthesis of (6aR,6bS,7S,8aS,8bS,11aR,12aS,12bS)-10-(4-((6-aminospiro[3.3]heptan-2-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-32)

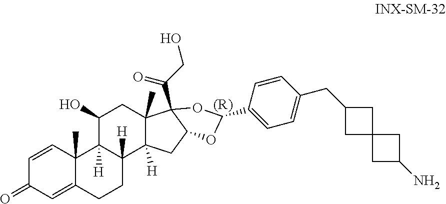

### Procedure

A 35 mL vial was charged with tert-butyl (6-(4-formylbenzyl) spiro[3.3]heptan-2-yl)carbamate (INX-SM-32-4) (0.16 g, 0.48 mmol), (8S,9S,10R,11S,13S,14S,16R,17S)-11, 16, 17-trihydroxy-17-(2-hydroxyacetyl)-10, 13-dimethyl-6,7,8,9, 10, 11, 12, 13, 14, 15, 16, 17-dodecahydro-3H-cyclopenta[a]phenanthren-3-one (16-alpha-hydroxyprednisolone) (0.13 g, 0.34 mmol), MgSO₄ (0.29 g, 2.43 mmol) and DCM (4 mL). To this solution, HClO₄ (0.40 g, 2.43 mmol) was added and stirred for another 2 h at room temperature. After completion of reaction as indicated by TLC, reaction mixture was quenched with sat. NaHCO₃ solution and concentrated over vacuum. The crude was triturated with cold water and precipitated solid was filtered and dried under vacuum.

The crude was purified by prep-HPLC (Column: SUNFIRE Prep C18 OBD, 19×250 mm, 5 μm, Mobile phase: A=0.1% FA in water, B=acetonitrile, A:B, 80:20), Retention time 18.54 min to give R-Isomer as white solid (0.045 g, 15.76%); LCMS: 588.4 (M+H)⁺; ¹H NMR (400 MHZ, MeOD, Key proton assignment): δ: 5.45 (s, 1H, Acetal-H), 5.05 (d, J=4.8 Hz, 1H, C16H).

### Synthesis of (6aR,6bS,7S,8aS,8bS,11aR,12aS,12bS)-10-(4-((5-oxa-2-azaspiro[3.4]octan-7-yl)methyl)phenyl)-7-hydroxy-8b-(2-hydroxyacetyl)-6a,8a-dimethyl-1,2,6a,6b,7,8,8a,8b, 11a,12,12a,12b-dodecahydro-4H-naphtho[2′,1′:4,5]indeno[1,2-d][1,3]dioxol-4-one (INX-SM-31)

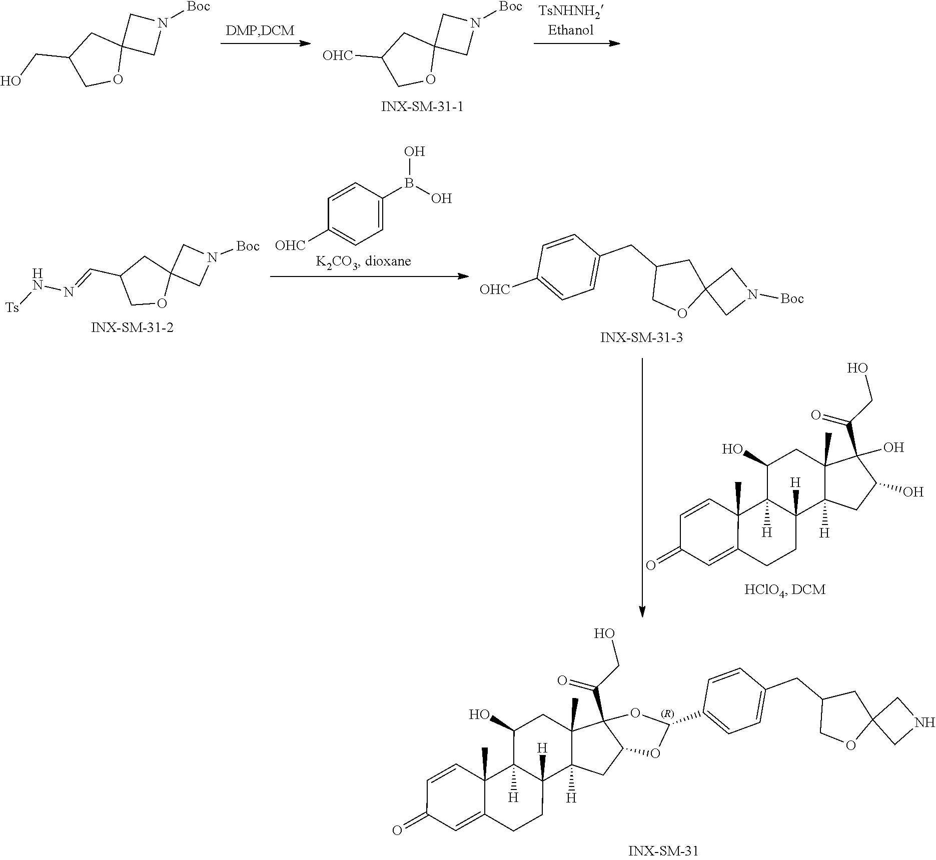

### Synthesis of tert-butyl 7-formyl-5-oxa-2-azaspiro[3.4]octane-2-carboxylate (INX-SM-31-1)

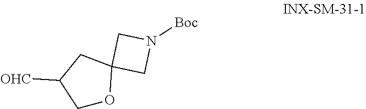 [SMILES: CC(C)(C)OC(=O)N1CC2(CC(C=O)CO2)C1]

### Procedure

A 100 mL three-necked round bottom flask was charged with tert-butyl 7-(hydroxymethyl)-5-oxa-2-azaspiro[3.4]octane-2-carboxylate (1.0 g, 4.11 mmol) and DCM (20 mL) under nitrogen. To this solution, Dess-Martin periodinane (DMP) (3.40, 8.22 mmol) was added at room temperature and stirred for 30 min. After completion of reaction as indicated by TLC, reaction mixture was quenched with saturated NaHCO₃ solution and extracted with ethyl acetate. The combined organic layer was dried over Na₂SO₄ and evaporated under vacuum to give title compound as gummy solid (0.8 g, 78.71%). ¹H NMR (DMSO-d6) δ: 9.59 (s, 1H), 4.08-4.04 (m, 1H), 3.88-3.70 (m, 5H), 3.21-3.19 (m, 1H), 2.37-2.21 (m, 2H), 1.36 (s, 9H).

### Synthesis of tert-butyl-7-((2-tosylhydrazono)methyl)-5-oxa-2-azaspiro[3.4]octane-2-carboxylate (INX-SM-31-2)

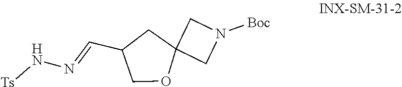 [SMILES: Cc1ccc(S(=O)(=O)N/N=C/C2COC3(C2)CN(C(=O)OC(C)(C)C)C3)cc1]

### Procedure

A 50 mL single-necked round bottom flask was charged with tert-butyl 7-formyl-5-oxa-2-azaspiro[3.4]octane-2-carboxylate (INX-SM-31-1) (0.8 g, 4.04 mmol) and EtOH (30 mL) under nitrogen. To this solution, p-toluenesulfonylhydrazide (0.92 g, 4.97 mmol) and catalytic amount of AcOH were added and stirred for 2 h at room temperature. After completion of reaction as indicated by TLC, reaction mixture was poured into water and the solid was filtered and dried under vacuum. The crude was purified by silica gel column chromatography (ethyl acetate/hexane, 20:80) to give title compound as white solid (0.7 g, 51.56%). LCMS: 410.8 (M+H)⁺.

### Synthesis of tert-butyl 7-(4-formylbenzyl)-5-oxa-2-azaspiro[3.4]octane-2-carboxylate (INX-SM-31-3)

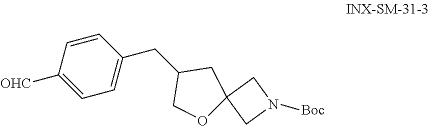 [SMILES: CC(C)(C)OC(=O)N1CC2(CC(Cc3ccc(C=O)cc3)CO2)C1]

### Procedure

A 35 mL vial was charged with tert-butyl-7-((2-tosylhydrazono)methyl)-5-oxa-2-azaspiro[3.4]octane-2-carboxylate (INX-SM-31-2) (0.72 g, 1.76 mmol) and dioxane (10 mL) under nitrogen. To this solution, (4-formylphenyl)boronic acid (0.26 g, 1.76 mmol) and K₂CO3 (0.48 g, 3.52 mmol) were added at room temperature and stirred at 110° C. for another 1 h. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane, 15:85) to give title compound as white solid (0.30 g, 52.96%). LCMS: 332.8 (M+H)⁺.

### Synthesis of (6aR,6bS,7S,8aS,8bS,11aR,12aS,12bS)-10-(4-((5-oxa-2-azaspiro[3.4]octan-7-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-31)

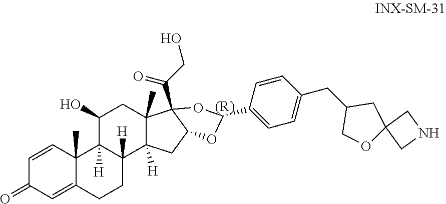

### Procedure

A 10 mL single-necked round bottom flask was charged with tert-butyl 7-(4-formylbenzyl)-5-oxa-2-azaspiro[3.4]octane-2-carboxylate (INX-SM-31-3) (0.30 g, 0.90 mmol), (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (16-alpha-hydroxyprednisolone) (0.34 g, 0.90 mmol), MgSO₄ (0.54 g, 4.52 mmol) and DCM (5 mL). To this solution, was added HClO₄ (0.45 g, 4.52 mmol) and stirred for another 1 h at room temperature. After completion of reaction as indicated by TLC, reaction mixture was poured into water and extracted with ethyl acetate.

The combined organic layer was dried over Na₂SO₄ and evaporated under vacuum. The crude was purified by The crude was purified by prep-HPLC (Column: SUNFIRE Prep C18 OBD, 19×250 mm, 5 μm, Mobile phase: A=0.1% FA in Water, B=ACN: MEOH: IPA (65:25:10); Retention time: 16.40 min to give R-Isomer as white solid 0.022 g, 4.50%); LCMS: 591.3 (M+H)⁺; ¹H NMR (400 MHZ, MeOD, Key proton assignment): δ: 5.44 (s, 1H, Acetal-H), 5.06 (d, J=4.8 Hz, 1H, C16H).

### Synthesis of (6aR,6bS,7S,8aS,8bS,10R,11aR,12aS,12bS)-10-(4-((3-aminooxetan-3-yl)methyl)phenyl)-7-hydroxy-8b-(2-hydroxyacetyl)-6a,8a-dimethyl-1,2,6a,6b,7,8,8a,8b,11a,12,12a,12b-dodeca hydro-4H-naphtho[2′,1′:4,5]indeno[1,2-d][1,3]dioxol-4-one (INX-SM-33)

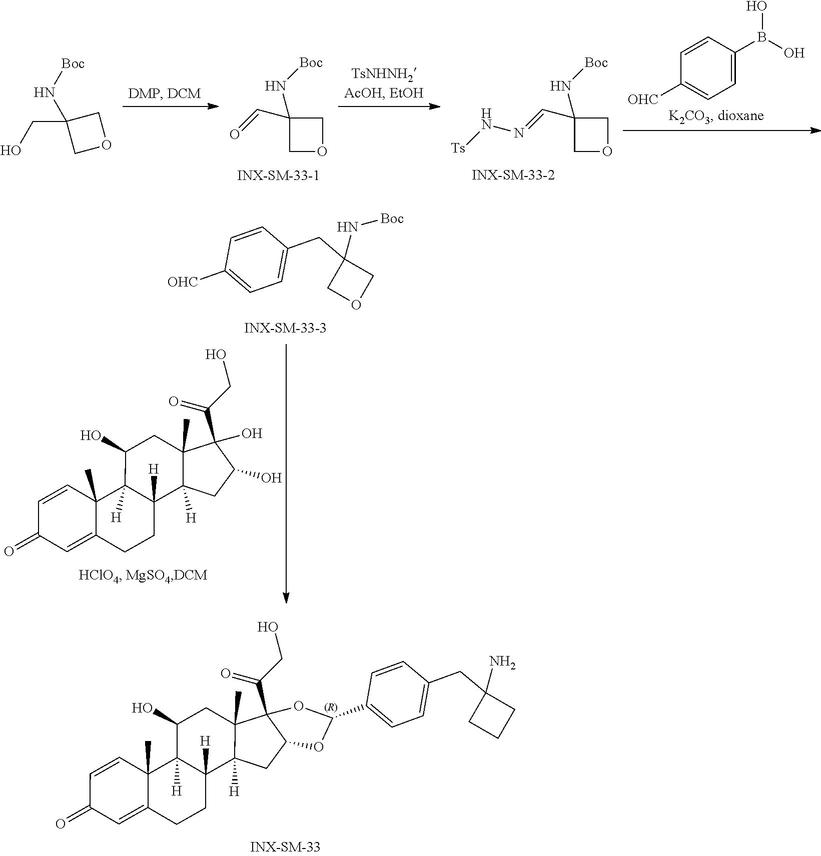

### Synthesis of tert-butyl (3-formyloxetan-3-yl)carbamate (INX-SM-33-1)

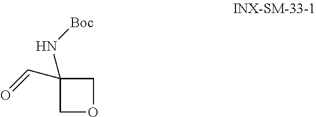 [SMILES: CC(C)(C)OC(=O)NC1(C=O)COC1]

### Procedure

A 100 mL three-necked round bottom flask was charged with tert-butyl (3-(hydroxymethyl)oxetan-3-yl)carbamate (2.0 g, 9.84 mmol) and DCM (20 mL) under nitrogen. To this solution, Dess-Martin periodinane (DMP) (4.17 g, 9.84 mmol) was added at 0° C. and stirred for 2 h. After completion of reaction as indicated by TLC, reaction mixture was filtered through celite and washed with diethyl ether. The combined organic layer was evaporated under vacuum to give crude product. The crude was purified by silica gel column chromatography (ethyl acetate/hexane: 45:55) to give the title compound as yellow solid (2.0 g, quantitative). ¹H NMR (CDCl3) δ: 9.85 (s, 1H), 5.50-5.42 (m, 1H), 5.10-4.940 (m, 1H), 4.86-4.84 (d, 2H), 1.47 (s, 9H).

### Synthesis of tert-butyl (3-((2-tosylhydrazono)methyl)oxetan-3-yl)carbamate (INX-SM-33-2)

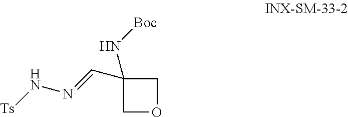 [SMILES: Cc1ccc(S(=O)(=O)N/N=C/C2(NC(=O)OC(C)(C)C)COC2)cc1]

### Procedure

A 50 mL single-necked round bottom flask was charged with tert-butyl (3-formyloxetan-3-yl)carbamate (INX-SM-33-1) (1.7 g, 8.44 mmol) and EtOH (17 mL) under nitrogen. To this solution, p-toluenesulfonylhydrazide (1.57 g, 8.44 mmol) and catalytic AcOH were added and stirred for 2 h at room temperature. After completion of reaction as indicated by TLC, reaction mixture was poured into water and extracted with ethyl acetate. The combined organic layer was dried over Na₂SO₄ and evaporated under vacuum to give crude product. The crude was purified by silica gel column chromatography (ethyl acetate/hexane, 50:50) to give title compound as white solid (2.5 g, 80.10%). LCMS: 387.4 (M+18).

### Synthesis of tert-butyl (3-(4-formylbenzyl) oxetan-3-yl)carbamate (INX-SM-33-3)

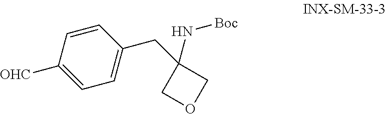 [SMILES: CC(C)(C)OC(=O)NC1(Cc2ccc(C=O)cc2)COC1]

### Procedure

A 50 mL vial was charged with tert-butyl (3-((2-tosylhydrazono)methyl)oxetan-3-yl)carbamate (INX-SM-33-2) (2.5 g, 6.77 mmol) and dioxane (25 mL) under nitrogen. To this solution, (4-formylphenyl)boronic acid (1.0 g, 6.77 mmol) and K₂CO3 (1.4 g, 10.16 mmol) were added at room temperature and stirred for another 2 h at 100° C. After completion of reaction as indicated by TLC, reaction mixture was poured into water and extracted with ethyl acetate. The combined organic layer was dried over Na₂SO₄ and evaporated under vacuum to give crude product. The crude was purified by silica gel column chromatography (ethyl acetate/hexane, 30:70) to give title compound as yellow solid (0.25 g, 12%). LCMS: 292.2 (M+H)⁺.

### (6aR,6bS,7S,8aS,8bS,10R,11aR,12aS,12bS)-10-(4-((3-aminooxetan-3-yl)methyl)phenyl)-7-hydroxy-8b-(2-hydroxyacetyl)-6a,8a-dimethyl-1,2,6a,6b,7,8,8a,8b,11a,12,12a,12b-dodeca hydro-4H-naphthol[2′,1′:4,5]indeno[1,2-d][1,3]dioxol-4-one (INX-SM-3.3)

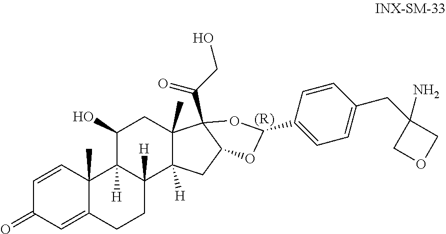

### Procedure

A 35 mL vial was charged with tert-butyl (3-(4-formylbenzyl) oxetan-3-yl)carbamate (INX-SM-33-1) (0.080 g, 0.27 mmol), (8S,9S,10R,11S,13S,14S,16R,17S)-11,16,17-trihydroxy-17-(2-hydroxyacetyl)-10, 13-dimethyl-6,7,8,9, 10, 11, 12, 13, 14, 15, 16, 17-dodecahydro-3H-cyclopenta[a]phenanthren-3-one (16-alpha-hydroxyprednisolone) (0.073 g, 0.19 mmol), MgSO₄ (0.16 g, 1.37 mmol) and DCM (2 mL). To this solution, HClO₄ (0.23 g, 1.37 mmol) was added and stirred for another 4 h at room temperature. After completion of reaction as indicated by TLC, reaction mixture was quenched with sat. NaHCO₃ solution and concentrated under vacuum. The crude was triturated with cold water and precipitated solid was filtered and dried under vacuum. The crude was purified by prep-HPLC (Column: SUNFIRE Prep C18 OBD, 19×250 mm, 5 μm, Mobile phase: A=0.1% FA IN WATER, B=Acetonitrile; A:B,80:20); Retention time: 8.70 min to give R-isomer of title compound as white solid (0.004 g, 2.65%) LCMS: 551.3 (M+H)⁺; ¹H NMR (400 MHZ, MeOD, Key proton assignment): δ: 5.48 (s, 1H, Acetal-H), 5.07 (d, J=5.4 Hz, 1H, C16H).

### Synthesis of (6aR,6bS,7S,8aS,8bS,11aR,12aS,12bS)-10-(4-((4-aminobicyclo[2.2.2]octan-1-yl)methyl)phenyl)-7-hydroxy-8b-(2-hydroxyacetyl)-6a,8a-dimethyl-1, 2, 6a,6b,7,8,8a,8b,11a,12,12a,12b-dodecahydro-4H-naphtho[2′,1′:4,5]indeno[1,2-d][1,3]dioxol-4-one (INX-SM-10)

### Reaction Scheme

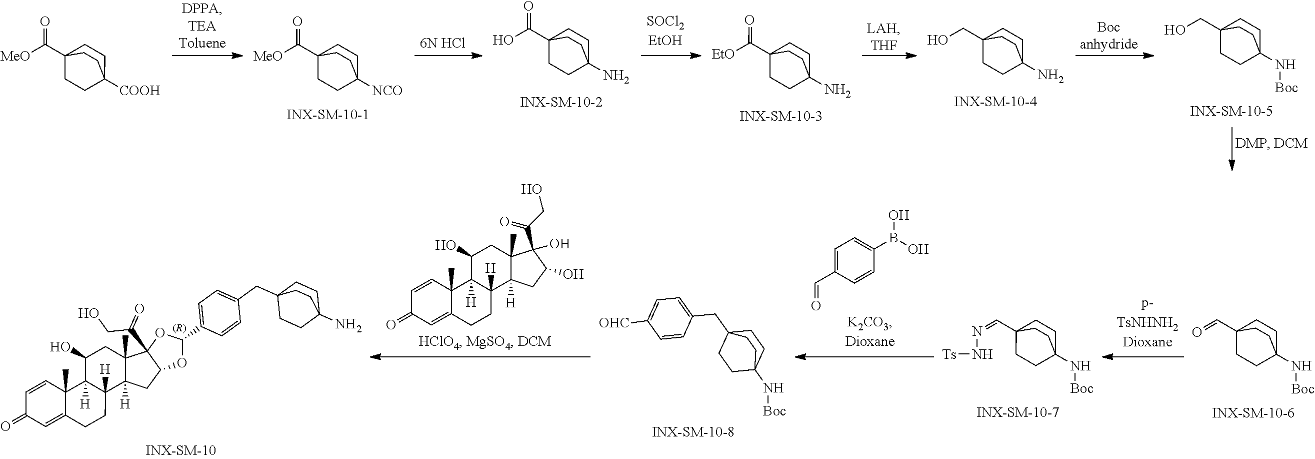

### Synthesis of methyl 4-isocyanatobicyclo[2.2.2]octane-1-carboxylate (INX-SM-10-1)

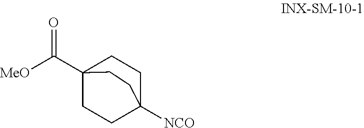

### Procedure

A 50 mL single-necked round bottom flask was charged with 4-(methoxy carbonyl) bicyclo[2.2.2]octane-1-carboxylic acid (1 g, 4.47 mmol) and toluene (20 mL). To this solution, diphenylphosphoryl azide (DPPA) (1.29 g, 4.47 mmol) and triethyl amine (0.47 g, 4.47 mmol) were added. The reaction mixture was heated at 110° C. for 2 h. After completion of reaction as indicated by TLC, reaction mixture was cooled at room temperature, diluted with ethyl acetate and washed with 10% citric acid solution and then saturated bicarbonate solution. The combined organic layer was dried over Na₂SO₄ and evaporated under vacuum. The crude was purified by silica gel column chromatography (ethyl acetate/hexane, 10:90) to give title compound as colorless liquid. (0.45 g, 45.46%). ¹H NMR (CDCl₃) δ: 3.62 (s, 3H), 1.90-1.87 (m, 12H).

### Synthesis of 4-aminobicyclo[2.2.2]octane-1-carboxylic acid (INX-SM-10-2)

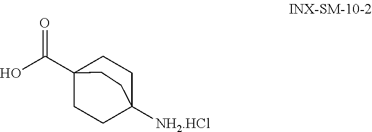

### Procedure

A 25 mL single-necked round bottom flask was charged with methyl 4-isocyanatobicyclo[2.2.2]octane-1-carboxylate (INX-SM-10-1) (0.45 g, 2.15 mmol) and 6N HCl (10 mL). The reaction mixture was stirred at room temperature for another 12 h. After completion of reaction as indicated by TLC, reaction mixture was evaporated under vacuum to give crude product containing title compound. The crude was triturated with n-pentene and diethyl ether to give white solid (0.45 g, quantitative). ¹H NMR (DMSO-d6) δ: 12.21 (bs, 1H), 8.20 (s, 3H), 1.83-1.69 (m, 12H).

### Synthesis of ethyl 4-aminobicyclo[2.2.2]octane-1-carboxylate (INX-SM-10-3)

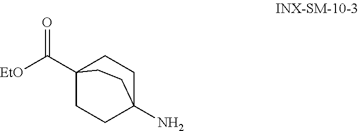 [SMILES: CCOC(=O)C12CCC(N)(CC1)CC2]

### Procedure

A 25 mL three-necked round bottom flask was charged with ethanol (5 mL) under nitrogen. To this solution, thionyl chloride (0.62 g, 5.32 mmol) was added at 0° C. and 4-aminobicyclo[2.2.2]octane-1-carboxylic acid (INX-SM-10-2) (0.45 g, 2.65 mmol) was added and refluxed for 3 h. After completion of reaction as indicated by TLC, reaction mixture was evaporated under vacuum to give crude product. The crude was purified by trituration with n-pentene and diethyl ether to give title compound as white solid (0.55 g, quantitative). LCMS: 198.20; ¹H NMR (DMSO-d6) δ: 8.13 (s, 1H), 7.68 (s, 2H), 4.04-4.99 (q, J=6.8 Hz, 2H), 1.82-1.71 (m, 12H) 1.16-1.12 (t, 3H, J=8 Hz).

### Synthesis of (4-aminobicyclo[2.2.2]octan-1-yl) methanol (INX-SM-10-4)

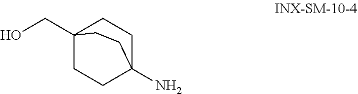 [SMILES: NC12CCC(CO)(CC1)CC2]

### Procedure

A 25 mL three-necked round bottom flask was charged with ethyl 4-aminobicyclo[2.2.2]octane-1-carboxylate (INX-SM-10-3) (0.55 g, 2.27 mmol) and THF (5.5 mL) under nitrogen. To this solution, LiAlH₄ (1M in THF) (6.9 mL, 6.9 mmol) was added at −20° C. and stirred at room temperature for 2 h. After completion of reaction as indicated by TLC, reaction mixture was quenched with 10% NaOH solution and filtered through celite bed. The filtrate was dried over Na₂SO₄ and evaporated under vacuum. The crude was triturated with n-pentene and diethyl ether to give title compound as white solid (0.30 g, 69.32%). LCMS: 156.1 (M+H)⁺; ¹H NMR (DMSO-d6) δ: 3.05 (s, 2H), 1.48-1.37 (m, 12H).

### Synthesis of tert-butyl (4-(hydroxymethyl)bicyclo[2.2.2]octan-1-yl)carbamate (INX-SM-10-5)

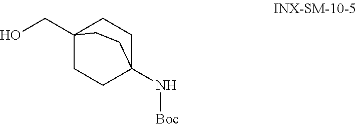 [SMILES: CC(C)(C)OC(=O)NC12CCC(CO)(CC1)CC2]

### Procedure

A 25 mL single-necked round bottom flask was charged with (4-aminobicyclo

octan-1-yl) methanol (INX-SM-10-4) (0.30 g, 1.93 mmol) and DCM (15 mL) under nitrogen. To this solution, Boc-anhydride (0.63 g, 2.90 mmol) was added at room temperature and stirred for another 16 h. After completion of reaction as indicated by TLC, reaction mixture was poured into water and extracted with ethyl acetate. The combined organic layer was washed with saturated bicarbonate solution, dried over Na₂SO₄ and evaporated under vacuum. The crude was triturated with diisopropyl ether to give title compound as white solid (0.45 g, 91.19%). LCMS: 200.2 (M+H-56); ¹H NMR (CDCl3) δ: 4.34 (bs, 1H), 3.27 (s, 2H), 1.86=1.82 (m, 6H), 1.59-1.53 (m, 6H), 1.43 (s, 9H).

### Synthesis of tert-butyl (4-formylbicyclo[2.2.2]octan-1-yl)carbamate (INX-SM-10-6)

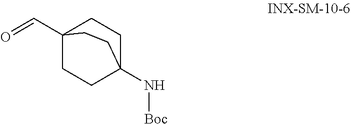 [SMILES: CC(C)(C)OC(=O)NC12CCC(C=O)(CC1)CC2]

### Procedure

A 25 mL single-necked round bottom flask was charged with tert-butyl (4-(hydroxymethyl)bicyclo[2.2.2]octan-1-yl)carbamate (INX-SM-10-5) (0.45 g, 1.76 mmol) and THF (10 mL). Dess-Martin periodinane (DMP) (1.12 g, 2.64 mmol) was added at room temperature and stirred for 1.5h at room temperature. After completion of reaction as indicated by TLC, reaction mixture was quenched with aqueous NaHCO₃ solution and extracted with ethyl acetate. The combined organic layer was washed with brine, dried over Na₂SO₄ and evaporated under vacuum to give title compound as white solid (0.45 g, crude). LCMS: 198.3 (M+H-56).

### Synthesis of tert-butyl (4-((2-tosylhydrazono)methyl)bicyclo[2.2.2]octan-1-yl) carbamate (INX-SM-10-7)

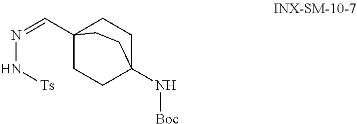 [SMILES: Cc1ccc(S(=O)(=O)N/N=C\C23CCC(NC(=O)OC(C)(C)C)(CC2)CC3)cc1]

### Procedure

A 10 mL glass vial was charged with tert-butyl (4-formylbicyclo[2.2.2]octan-1-yl) carbamate (INX-SM-10-6) (0.45 g, 1.77 mmol) and ethanol (5 mL). To this solution, p-toluenesulfonylhydrazide (0.39 g, 2.13 mmol) and acetic acid (0.05 g, 0.88 mmol) were added at room temperature and stirred for 1 h at room temperature. After completion of reaction as indicated by TLC, reaction mixture was poured into water. The white solid was filtered and dried under vacuum to give title compound as off white solid (0.38g 51.42%). LCMS: 422.3 (M+H)⁺; ¹H NMR (DMSO-d6) δ: 10.72 (s, 1H), 7.65 (d, J=8.4 Hz, 2H), 7.39 (d, J=8.0 Hz, 2H) 7.02 (s, 1H), 6.37 (bs, 1H), 2.37 (s, 3H), 1.71-1.69 (m, 6H), 1.46-1.42 (m, 6H), 1.34 (s, 9H).

### Synthesis of tert-butyl (4-(4-formylbenzyl)bicyclo[2.2.2]octan-1-yl)carbamate (INX-SM-10-8)

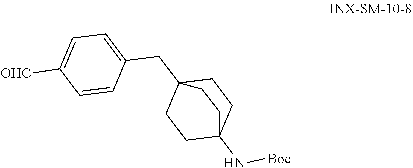 [SMILES: CC(C)(C)OC(=O)NC12CCC(Cc3ccc(C=O)cc3)(CC1)CC2]

A 50 mL single-necked round bottom flask was charged with tert-butyl (4-((2-tosylhydrazono)methyl)bicyclo[2.2.2]octan-1-yl)carbamate (INX-SM-10-7) (1.0 g, 2.37 mmol) and dioxane (20 mL). (4-Formylphenyl)boronic acid (0.53 g, 3.55 mmol) and K₂CO3 (0.49 g, 3.55 mmol) were added at room temperature and stirred for another 2 h at 110° C. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane: 50:50) to give title compound as colorless liquid (0.06 g, 7.36%). LCMS: 288.8 (M+H-56).

### Synthesis of (6aR,6bS,7S,8aS,8bS,11aR,12aS,12bS)-10-(4-((4-aminobicyclo[2.2.2]octan-1-yl)methyl)phenyl)-7-hydroxy-8b-(2-hydroxyacetyl)-6a,8a-dimethyl-1, 2, 6a,6b,7,8,8a,8b,11a,12,12a,12b-dodecahydro-4H-naphtho[2′,1′:4,5]indeno[1,2-d][1,3]dioxol-4-one (INX-SM-10)

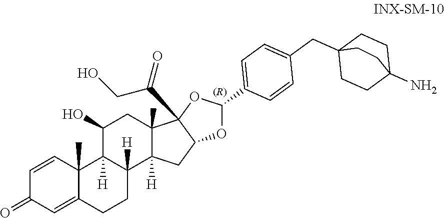

### Procedure

A 10 mL single-necked round bottom flask was charged with tert-butyl (4-(4-formylbenzyl)bicyclo[2.2.2]octan-1-yl)carbamate (INX-SM-10-8) (0.05 g, 0.145 mmol) and (8S,9S,10R,11S,13S,14S,16R,17S)-11,16,17-trihydroxy-17-(2-hydroxyacetyl)-10,13-dimethyl-6,7, 8, 9, 10, 11, 12, 13, 14, 15, 16, 17-dodecahydro-3H-cyclopenta[a]phenanthren-3-one (16-alpha-hydroxyprednisolone) (0.054 g, 0.14 mmol), MgSO₄ (0.080 g, 0.73 mmol) and DCM (5 mL). To this solution, HClO₄ (0.072 g, 0.73 mmol) was added and stirred for another 1 h at room temperature. After completion of reaction as indicated by TLC, reaction mixture was quenched with saturated bicarbonate solution and extracted with ethyl acetate. The combined organic layer was dried over Na₂SO₄ and evaporated under vacuum. The crude was purified by prep-HPLC (Column: SUNFIRE Prep C18 OBD, 19×250 mm, 5 μm, Mobile phase: A=0.1% FA in water, B=ACN: MEOH: IPA (65:25:10); A:B, 80:20); Retention time 18.76 min to give R-Isomer of title compound as white solid (0.015 g, 14.27%); LCMS 603.52 (M+H)⁺; ¹H NMR (400 MHZ, MeOD Key proton assignment) δ: 5.46 (s, 1H, Acetal-H), 5.06 (d, J=4.80 Hz, 1H, C16H).

### Synthesis of (6aR,6bS,7S,8aS,8bS,10R,11aR,12aS,12bS)-10-(4-((1-amino-3,3-difluorocyclo butyl)methyl)phenyl)-7-hydroxy-8b-(2-hydroxyacetyl)-6a,8a-dimethyl 1,2,6a,6b,7,8,8a,8b,11a,12,12a,12b-dodecahydro-4H-naphtho[2′,1′:4,5]indeno[1,2-d][1,3]dioxol-4-one (INX-SM-35)

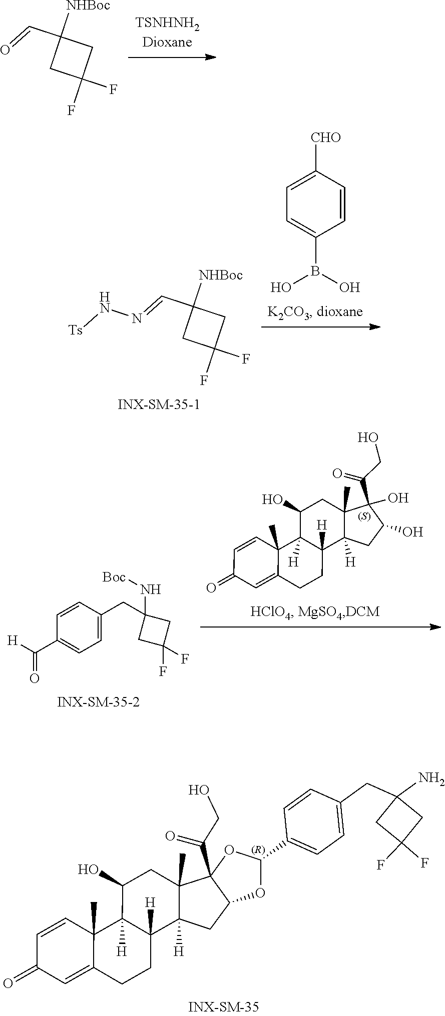

### Synthesis of tert-butyl (E)-(3,3-difluoro-1-((2-tosylhydrazono)methyl)cyclobutyl)carbamate (INX-SM-35-1)

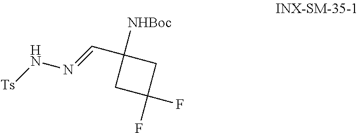 [SMILES: Cc1ccc(S(=O)(=O)N/N=C/C2(NC(=O)OC(C)(C)C)CC(F)(F)C2)cc1]

### Procedure

A 30 mL glass vial was charged with tert-butyl (3,3-difluoro-1-formylcyclobutyl) carbamate (0.50 g, 2.12 mmol) and dioxane (5 mL) under nitrogen. To this solution, p-toluenesulfonylhydrazide (0.4 g, 2.12 mmol) was added and stirred for 2 h at 90° C. After completion of reaction as indicated by TLC, reaction mixture was poured into water and extracted with ethyl acetate. The combined organic layer was dried over Na₂SO₄ and evaporated under vacuum to give crude product. The crude was purified by silica gel column chromatography (ethyl acetate/hexane, 30:70) to give title compound as light-yellow solid (0.55 g, 64.23%). LCMS: 348.1 (M+H-56).

### Synthesis of tert-butyl (3,3-difluoro-1-(4-formylbenzyl)cyclobutyl)carbamate (INX-SM-35-2)

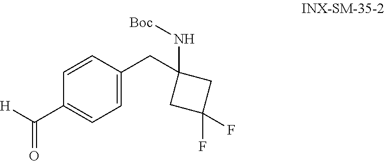 [SMILES: CC(C)(C)OC(=O)NC1(Cc2ccc(C=O)cc2)CC(F)(F)C1]

### Procedure

A 30 mL vial was charged with tert-butyl (3,3-difluoro-1-((2-tosylhydrazono) methyl)cyclobutyl)carbamate (INX-SM-35-1) (0.50 g, 1.72 mmol) and dioxane (5 mL) under nitrogen. To this solution, (4-formylphenyl)boronic acid (0.18 g, 1.72 mmol) and K₂CO3 (0.25 g, 1.85 mmol) were added at room temperature and stirred for another 2 h at 110° C. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane, 10:90) to give title compound as white solid (0.11 g, 24.80%). LCMS: 326.1 (M+H)⁺.

### Synthesis of (6aR,6bS,7S,8aS,8bS,10R,11aR,12aS,12bS)-10-(4-((1-amino-3,3-difluorocyclo butyl)methyl)phenyl)-7-hydroxy-8b-(2-hydroxyacetyl)-6a,8a-dimethyl 1,2,6a,6b,7,8,8a,8b,11a,12,12a,12b-dodecahydro-4H-naphtho[2′,1′:4,5]indeno[1,2-d][1,3]dioxol-4-one (INX-SM-35)

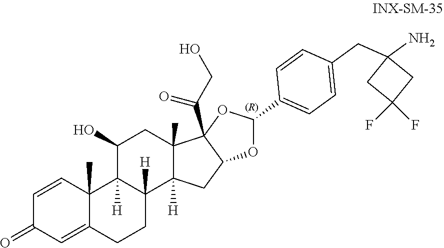

### Procedure

A 25 mL single-necked round bottom flask was charged with tert-butyl (3,3-difluoro-1-(4-formylbenzyl)cyclobutyl)carbamate (INX-SM-35-3) (0.11 g, 0.33 mmol), (8S,9S,10R,11S,13S,14S,16R,17S)-11,16,17-trihydroxy-17-(2-hydroxyacetyl)-10, 13-dimethyl-6,7,8,9,10,11,12,13,14, 15,16,17-dodecahydro-3H-cyclopenta[a]phenanthren-3-one (16-alpha-hydroxyprednisolone) (0.1 g, 0.27 mmol), MgSO₄ (0.2 g, 1.69 mmol) and DCM (3 mL). To this solution, HClO₄ (0.16 g, 1.69 mmol) was added and stirred for another 2 h at room temperature. After completion of reaction as indicated by TLC, reaction mixture was poured into water and extracted with ethyl acetate. The combined organic layer was dried over Na₂SO₄ and evaporated under vacuum. The crude was purified by prep-HPLC (Column: YMC-Actus Triart Prep C18-S, 250×20 mm S-5 μm, 12 nm, Mobile phase: A=0.05% ammonia in water, B=Acetonitrile; A:B, 58:42), Retention time 18.36 min to give R-Isomer (Fr-1) of title compound as white solid (0.030 g, 15.58%); LCMS: 585.4 (M+H)⁺; ¹H NMR (400 MHZ, MeOD, Key proton assignment): δ: 5.48 (s, 1H, Acetal-H), 5.07 (d, J=5.2 Hz, 1H, C16H).

### Synthesis of INX-A1

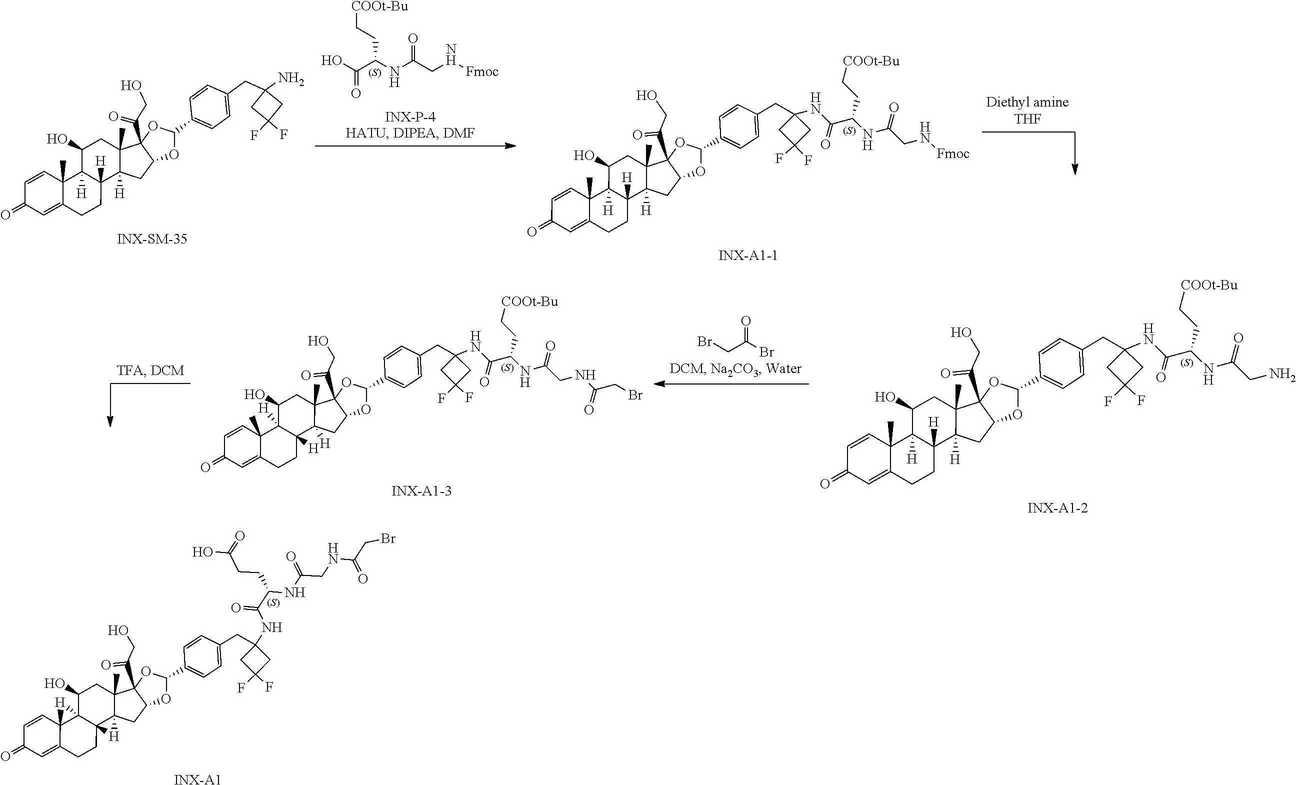

### Synthesis of tert-butyl(S)-4-(2-((((9H-fluoren-9-yl)methoxy)carbonyl)amino)acetamido)-5-((3,3-difluoro-1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)amino)-5-oxopentanoate (INX-AI-1)

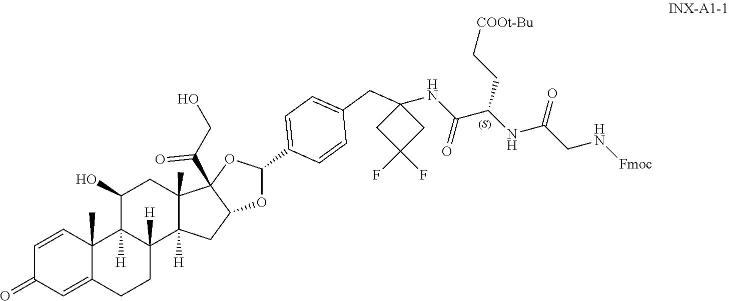

### Procedure

A 10 mL single-necked round bottom flask was charged with(S)-2-(2-((((9H-fluoren-9-yl)methoxy)carbonyl)amino)acetamido)-5-(tert-butoxy)-5-oxopentanoic acid (INX-P-4) (0.20 g, 0.41 mmol), HATU (0.24 g, 0.64 mmol), DMF (2 mL) and DIPEA (0.11 g, 0.82 mmol) at room temperature. To this solution, tert-butyl(S)-4-(2-((((9H-fluoren-9-yl)methoxy)carbonyl) amino)acetamido)-5-((3,3-difluoro-1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)amino)-5-oxopentanoate (INX-SM-35) (0.25 g, 0.41 mmol) was added and stirred for 1 h at room temperature. After completion of reaction as indicated by TLC, reaction mixture was poured into water and extracted with ethyl acetate. The combined organic layer was dried over Na₂SO₄ and evaporated under vacuum to give crude product. The crude was purified by reverse phase column chromatography (acetonitrile/water: 50:50) to give the title compound as pale yellow solid (0.24 g, 52.03%).

### Synthesis of tert-butyl(S)-4-(2-aminoacetamido)-5-((3,3-difluoro-1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)amino)-5-oxopentanoate (INX-AI-2)

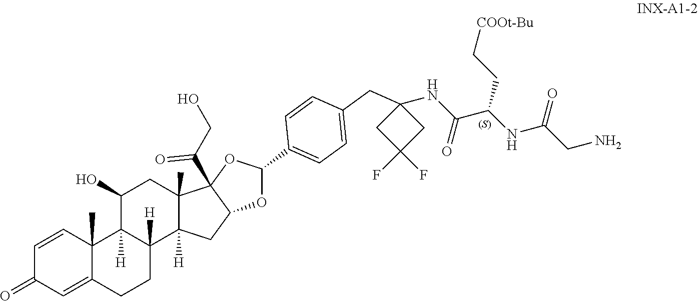

### Procedure

A 10 mL single-necked round bottom flask was charged with tert-butyl(S)-4-(2-((((9H-fluoren-9-yl)methoxy)carbonyl)amino)acetamido)-5-((3,3-difluoro-1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)amino)-5-oxopentanoate (INX-AI-1) (0.2 g, 0.12 mmol) and THF (3 mL). To this solution, diethyl amine (0.3 g, 0.24 mmol) was added at room temperature and stirred for 3 h. After completion of reaction as indicated by TLC, reaction mixture was evaporated under vacuum and triturated with diethyl ether and pentane to give title compound as yellow solid (0.13 g, 68.74%) LCMS: 827.6 (M+1).

### Synthesis of tert-butyl(S)-4-(2-(2-bromoacetamido)acetamido)-5-((3,3-difluoro-1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)amino)-5-oxopentanoate (INX-AI-3)

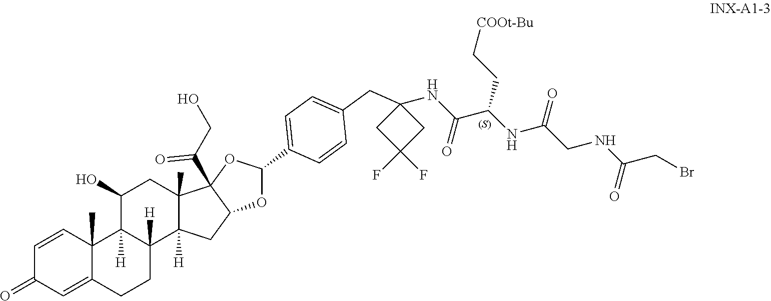

A 10 mL single-necked round bottom flask was charged with tert-butyl(S)-4-(2-aminoacetamido)-5-((3,3-difluoro-1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)amino)-5-oxopentanoate (INX-AI-2) (0.1 g, 0.12 mmol) and DCM (4 mL). To this solution, Na₂CO₃ (0.048 g, 0.24 mmol) solution in water (1 mL) and bromo acetyl bromide (0.005 g, 0.48 mmol) were added drop wise at room temperature and stirred for 1 h. After completion of reaction as indicated by TLC, reaction mixture was quenched with water and extracted with DCM. The combined organic layer was dried over Na₂SO₄ and evaporated under vacuum. The crude was purified by reverse phase column chromatography (acetonitrile/water: 50:50) to give title compound as pale yellow solid (0.10 g, 67.10%). LCMS: 946.8, 848.9 (M &M+2).

### Synthesis of (S)-4-(2-(2-bromoacetamido)acetamido)-5-((3,3-difluoro-1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)amino)-5-oxopentanoic acid (INX-AI-1)

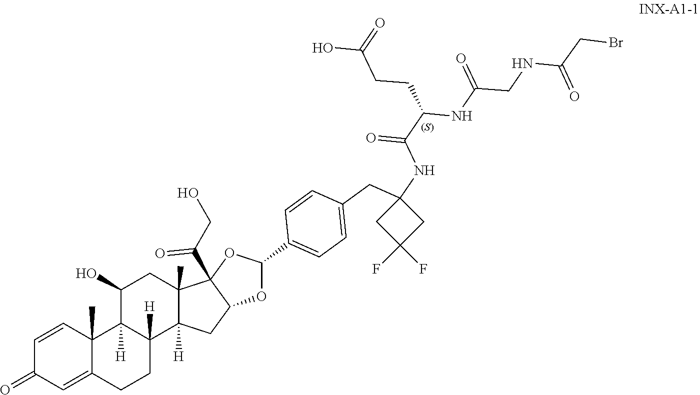

### Procedure

A 10 mL single-necked round bottom flask was charged with tert-butyl(S)-4-(2-(2-bromoacetamido)acetamido)-5-((3,3-difluoro-1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)amino)-5-oxopentanoate (INX-AI-3) (0.10 g, 0.01 mmol) in DCM (2 mL). To this solution, TFA (0.24 g, 2.10 mmol) was added at room temperature and stirred for 2 h at room temperature. After completion of reaction as indicated by TLC, reaction mixture was evaporated under vacuum to give title compound as off white solid. (0.090 g, 95.67%). LCMS: 890.90, 893.0 (M&M+2).

### Synthesis of (S)-4-(2-(2-bromoacetamido)acetamido)-5-((6-(4-11aR,12aS,12bS)-7-hydroxy-8b-(2-hydroxyacetyl)-6a,8a-((6aR,6bS,7S,8aS,8bS,10R, dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX—V)

### Reaction Scheme

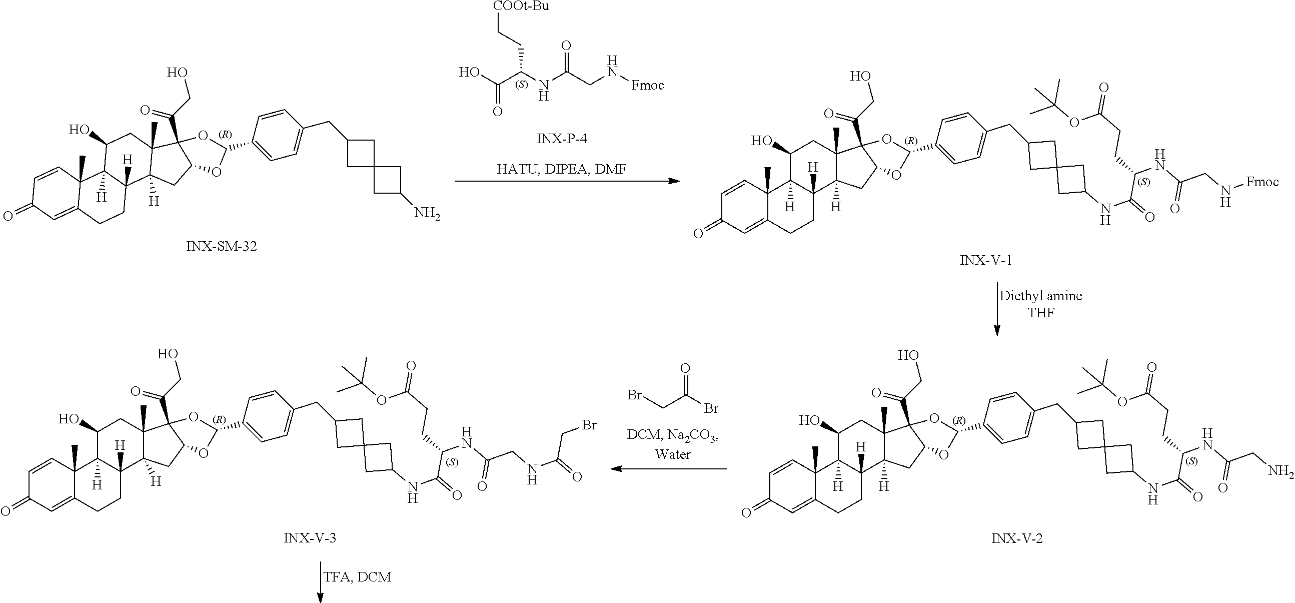

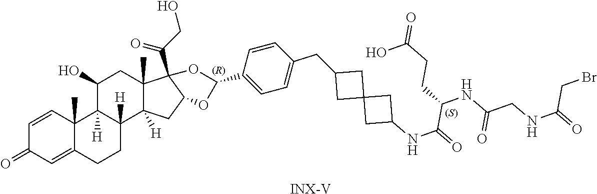

### Synthesis of tert-butyl(S)-4-(2-((((9H-fluoren-9-yl)methoxy)carbonyl)amino)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX—V-1)

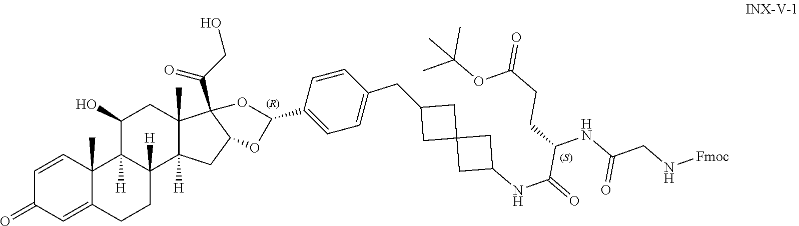

### Procedure

A 10 mL single-necked round bottom flask was charged with(S)-2-(2-((((9H-fluoren-9-yl)methoxy)carbonyl)amino)acetamido)-5-(tert-butoxy)-5-oxopentanoic acid (INX-P-4) (0.25 g, 0.41 mmol) and HATU (0.20 g, 0.41 mmol), DMF (2 mL) and DIPEA (0.10 g, 0.82 mmol) at room temperature. To this solution, (6aR,6bS,7S,8aS,8bS,10R,11aR,12aS,12bS)-10-(4-((6-aminospiro[3.3]heptan-2-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-32) (0.25 g, 0.41 mmol) was added and stirred for 1 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water: 50:50) to give title compound as pale-yellow solid. It was used immediately for next step.

### Synthesis of tert-butyl(S)-4-(2-aminoacetamido)-5-((6-(4-((6aR,6bS,7S,8aS,8bS, 10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX—V-2)

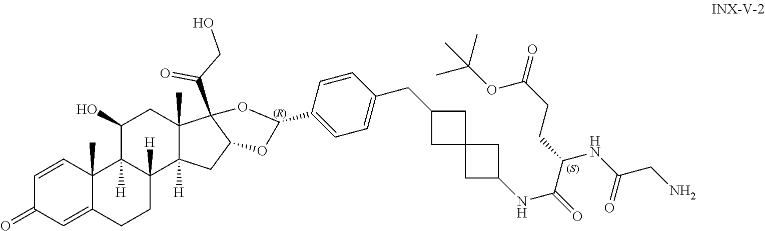

### Procedure

A 10 mL single-necked round bottom flask was charged with tert-butyl(S)-4-(2-((((9H-fluoren-9-yl)methoxy)carbonyl)amino)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX—V-1) (0.2 g, 0.19 mmol) and THF (2 mL). To this solution, diethyl amine (0.14 g, 1.9 mmol) was added at room temperature and stirred for 3 h at room temperature. After completion of reaction as indicated by TLC, reaction mixture was evaporated under vacuum and triturated with diethyl ether to give yellow solid (0.15 g, 90.12%). LCMS: 831.9 (M+H)⁺.

### Synthesis of tert-butyl(S)-4-(2-(2-bromoacetamido)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX—V-3)

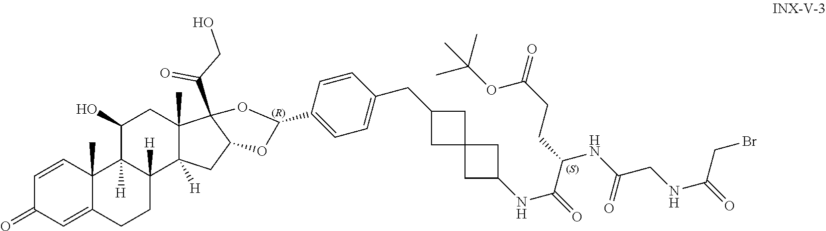

### Procedure

A 10 mL single-necked round bottom flask was charged with tert-butyl(S)-4-(2-aminoacetamido)-5-((6-(4-((6aR,6bS,7S,8aS,8bS,10R,11aR,12aS,12bS)-7-hydroxy-8b-(2-hydroxy acetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX—V-2) (0.15 g, 0.28 mmol) in DCM (3 mL). To this solution, Na₂CO₃ (0.11 g, 0.57 mmol) solution in water (1 mL) followed by bromoacetyl bromide (0.037 g, 0.18 mmol) was added dropwise at room temperature and stirred for 1 h. After completion of reaction as indicated by TLC, reaction mixture was quenched with water and extracted with DCM. The combined organic layer was dried over Na₂SO₄ and evaporated under vacuum. The crude was purified by reverse phase column chromatography (acetonitrile/water: 50:50) to give title compound as pale-yellow solid (0.070 g, 40%). LCMS: 950.9, 952.9 (M & M+2).

### Synthesis of (S)-4-(2-(2-bromoacetamido)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX—V)

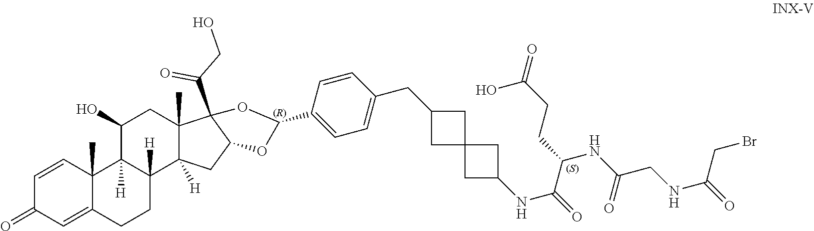

### Procedure

A 10 mL single-necked round bottom flask was charged with tert-butyl(S)-4-(2-(2-bromoacetamido)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX—V-3) (0.070 g, 0.07 mmol) and DCM (2 mL). To this solution, TFA (0.055 g, 0.71 mmol) was added and stirred for 2 h at room temperature. After completion of reaction as indicated by TLC, reaction mixture was evaporated under vacuum to give crude product as light-yellow solid. The crude was purified by prep-HPLC (Column: Xbridge Prep, C18, OBD 19×250 mm, 5 μm; Mobile phase: A=0.1% FA in Water, B=acetonitrile; A:B, 58:42) to give R-Isomer which was eluted at retention time 16.92 min to give title compound as off white solid (0.004 g, 11.83%). LCMS: 895.1 & 897.1 (M& M+2); ¹H NMR (400 MHZ, MeOD, Key proton assignment): δ: 5.44 (s, 1H, Acetal-H), 5.05 (d, J=4.8 Hz, 1H, C16H).

### Synthesis of (S)-6-amino-2-(2-(2-bromoacetamido)acetamido)-N-(3-(4-12aS,12bS)-7-hydroxy-8b-(2-hydroxyacetyl)-6a,8a-((6aR,6bS,7S,8aS,8bS,10R,11aR, dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicycle[1.1.1]pentan-1-yl) hexanamide (INX—W)

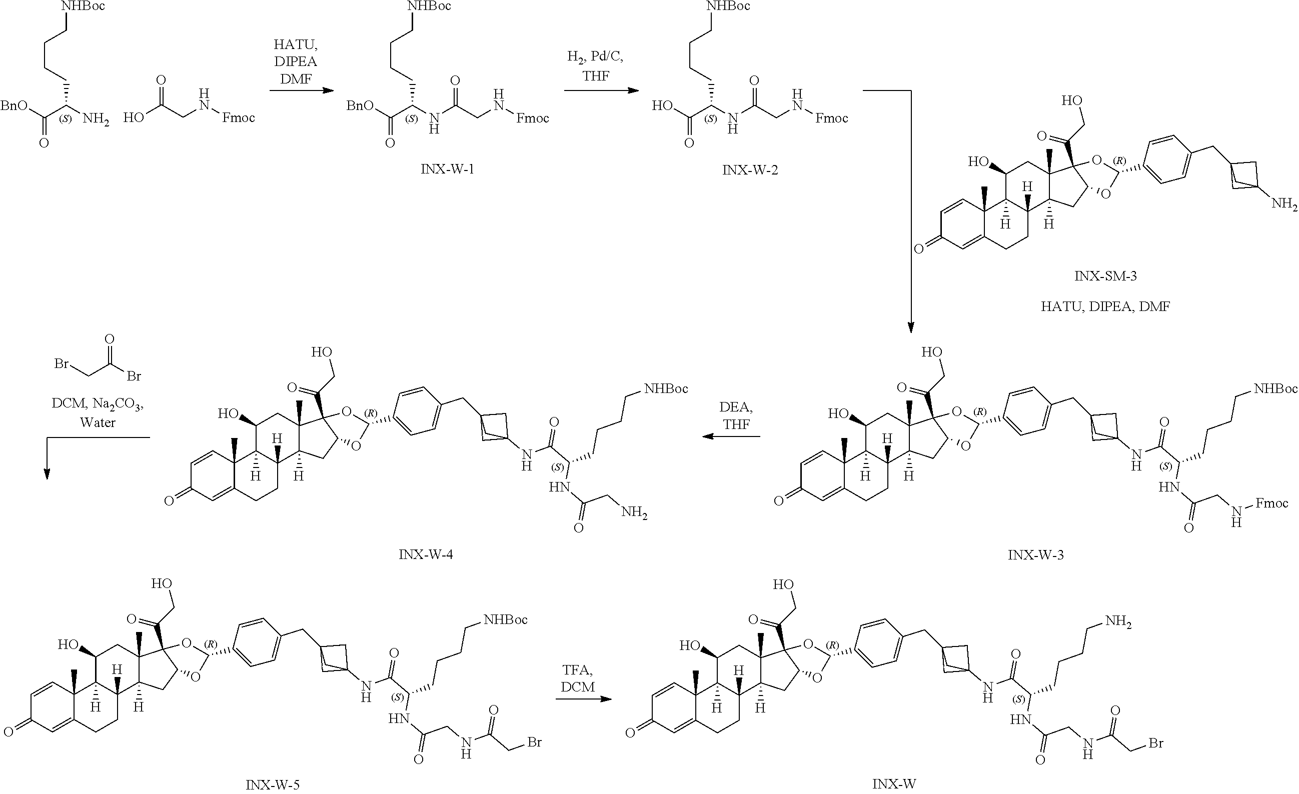

### Synthesis of benzyl N2-((((9H-fluoren-9-yl)methoxy)carbonyl) glycyl)-N6-(tert-butoxy carbonyl)-L-lysinate (INX—W-1)

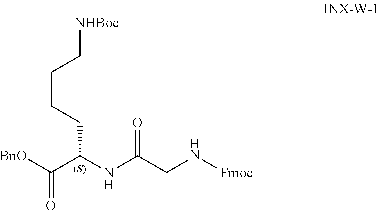

### Procedure

A 250 mL single-necked round bottom flask was charged with (((9H-fluoren-9-yl)methoxy)carbonyl) glycine (8.8 g, 29.62 mmol), HATU (16.9 g, 44.67 mmol), DMF (100 mL) and DIPEA (16 g, 89.28 mmol) at room temperature. To this solution, benzyl N6-(tert-butoxycarbonyl)-L-lysinate (10 g, 29.76 mmol) was added and stirred for 4 h at room temperature. After completion of reaction as indicated by TLC, reaction mixture was poured into water and extracted with ethyl acetate. The combined organic layer was dried over Na₂SO₄ and evaporated under vacuum. The crude was purified by column chromatography (ethyl acetate:hexane, 30:70) to give title compound as light-yellow solid (15 g, 81.96%). LCMS: 616.6 (M+H)⁺.

### Synthesis of N2-((((9H-fluoren-9-yl)methoxy)carbonyl) glycyl)-N6-(tert-butoxycarbonyl)-L-lysine (INX—W-2)

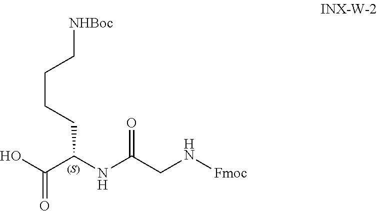

A 100 mL single-necked round bottom flask was charged with benzyl N2-((((9H-fluoren-9-yl)methoxy)carbonyl) glycyl)-N6-(tert-butoxycarbonyl)-L-lysinate (INX—W-1) (5 g, 8.12 mmol) and MeOH (50 mL). To this solution, 10% Pd/C (2.5 g) was added at room temperature and purged hydrogen for 2 h. After completion of reaction as indicated by TLC, reaction mixture was filtered through celite and filtrate was evaporated under vacuum. The crude was purified by reverse phase column chromatography (acetonitrile:water, 50:50) to give title compound as yellow solid (0.5 g, 23.43%). LCMS: 426.2 (M+1-Boc).

### Synthesis of (9H-fluoren-9-yl) methyl (2-(((S)-6-((tert-butoxycarbonyl)amino)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-1-oxohexan-2-yl)amino)-2-oxoethyl)carbamate (INX—W-3)

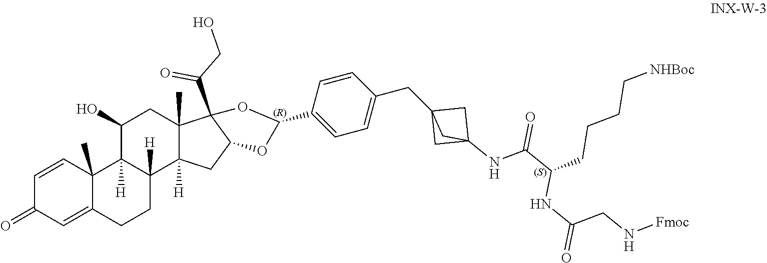

### Procedure

A 50 mL single-necked round bottom flask was charged with N2-((((9H-fluoren-9-yl)methoxy)carbonyl) glycyl)-N6-(tert-butoxycarbonyl)-L-lysine (INX—W-2) (0.5 g, 0.95 mmol), HATU (0.54 g, 1.42 mmol), DMF (25 mL) and DIPEA (0.5 mL, 2.85 mmol) at room temperature. To this solution, (6aR,6bS,7S,8aS,8bS,10R,11aR,12aS,12bS)-10-(4-((3-amino bicyclo[1.1.1]pentan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-3) (0.53 g, 0.95 mmol) was added and stirred for 4 h at room temperature. After completion of reaction as indicated by TLC, reaction mixture was poured into water and extracted with ethyl acetate. The combined organic layer was dried over Na₂SO₄ and evaporated under vacuum to give crude product. The crude was purified by reverse phase column chromatography (acetonitrile:water, 70:30) to give title compound as yellow solid (0.45 g, 44.32%). LCMS: 1067.7 (M+H)⁺.

### Synthesis of tert-butyl ((S)-5-(2-aminoacetamido)-6-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicycle[1.1.1]pentan-1-yl)amino)-6-oxohexyl)carbamate (INX—W-4)

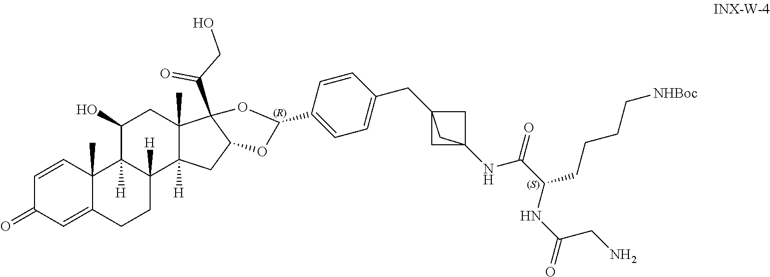

### Procedure

A 50 mL single-necked round bottom flask was charged with (9H-fluoren-9-yl) methyl (2-(((S)-6-((tert-butoxycarbonyl)amino)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodeca hydro-1H-naphtho[2′,1′:4,5]indeno[1,2-d][1,3]dioxol-10-yl)benzyl)bicyclo[1.1.1]pentan-1-yl)amino)-1-oxohexan-2-yl)amino)-2-oxoethyl)carbamate (INX—W-3) (0.45 g, 0.42 mmol) and THF (20 mL). To this solution, diethyl amine (0.30 g, 4.21 mmol) was added and stirred for 2 h at room temperature. After completion of reaction as indicated by TLC, reaction mixture was concentrated under vacuum. The crude was purified by trituration with diethyl ether-hexane and dried under vacuum to give title compound as yellow solid (0.35 g, 98.23%). LCMS: 845.6 (M+H)⁺.

### Synthesis of tert-butyl ((S)-5-(2-(2-bromoacetamido)acetamido)-6-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-6-oxohexyl)carbamate (INX—W-5)

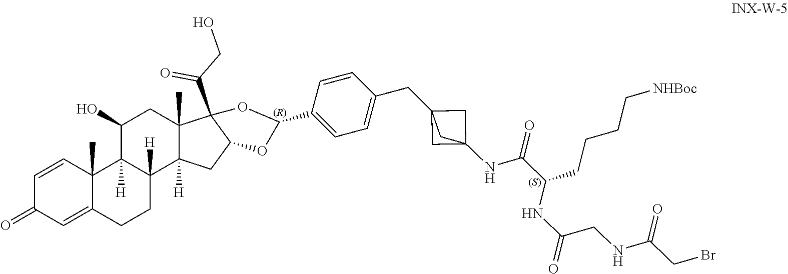

### Procedure

A 25 mL single-necked round bottom flask was charged with tert-butyl ((S)-5-(2-aminoacetamido)-6-((3-(4-((6aR,6bS,7S,8aS,8bS,10R,11aR,12aS,12bS)-7-hydroxy-8b-(2-hydroxy acetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicycle[1.1.1]pentan-1-yl)amino)-6-oxohexyl)carbamate (INX—W-4) (0.35 g, 0.41 mmol) and DCM (5 mL). To this solution, Na₂CO₃ (0.070 g, 0.82 mmol) in water (1 mL) followed by bromoacetyl bromide (0.1 g, 0.49 mmol) was added at room temperature and stirred for 1 h. After completion of reaction as indicated by TLC, reaction mixture was quenched with water and extracted with DCM. The combined organic layer was dried over Na₂SO₄ and evaporated under vacuum. The crude was purified by reverse phase column chromatography (acetonitrile/water, 50:50) to give title compound as off white solid (0.330 g, 82.48%). LCMS: 967.5 (M+H)⁺.

### Synthesis of (S)-6-amino-2-(2-(2-bromoacetamido)acetamido)-N-(3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicycle[1.1.1]pentan-1-yl) hexanamide (INX—W)

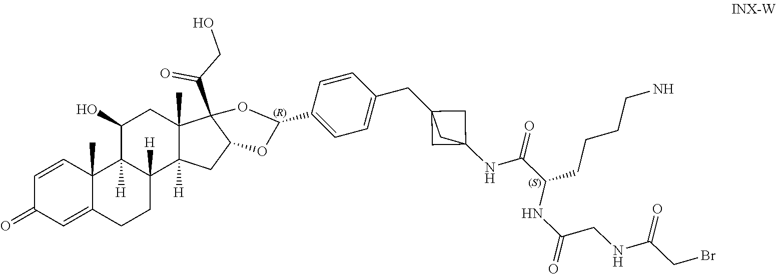

### Procedure

A 10 mL single-necked round bottom flask was charged with tert-butyl ((S)-5-(2-(2-bromoacetamido)acetamido)-6-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-6-oxohexyl)carbamate (INX—W-5) (0.10 g, 0103 mmol) and DCM (5 mL). To this solution, TFA (0.059 g, 0.52 mmol) was added and stirred for 2 h at room temperature. After completion of reaction as indicated by TLC, reaction mixture was evaporated under vacuum. The crude INX—W was purified by prep-HPLC (Column: SUNFIRE Prep C18 OBD, 19×250 mm, 5 μm, Mobile phase: A=0.1% FA in water, B=ACN: MeOH: IPA (65:25:10); A:B, 62:38); Retention time 19.06 min to give R-Isomer as white solid (0.008 g, 8.93%); ¹H NMR (400 MHz, MeOD, Key proton assignment): δ: 5.47 (s, 1H, Acetal-H), 5.07 (d, J=4.8 Hz, 1H, C16H).

### Synthesis of (S)-2-(2-bromoacetamido)-N—((S)-1-((3-(4-((6aR,6bS,7S,8aS,8bS,10R,11aR, 12aS,12bS)-7-hydroxy-8b-(2-hydroxyacetyl)-6a,8a-dimethyl-4-oxo-12,12a,12b-dodecahydro-1H-naphtho[2′,1′:4,5]indeno[1,2-bicyclo[1.1.1]pentan-1-yl)amino)-1-oxopropan-2-2,4,6a,6b,7,8,8a,8b,11a, d][1,3]dioxol-10-yl)benzyl) yl) propanamide (INX—R)

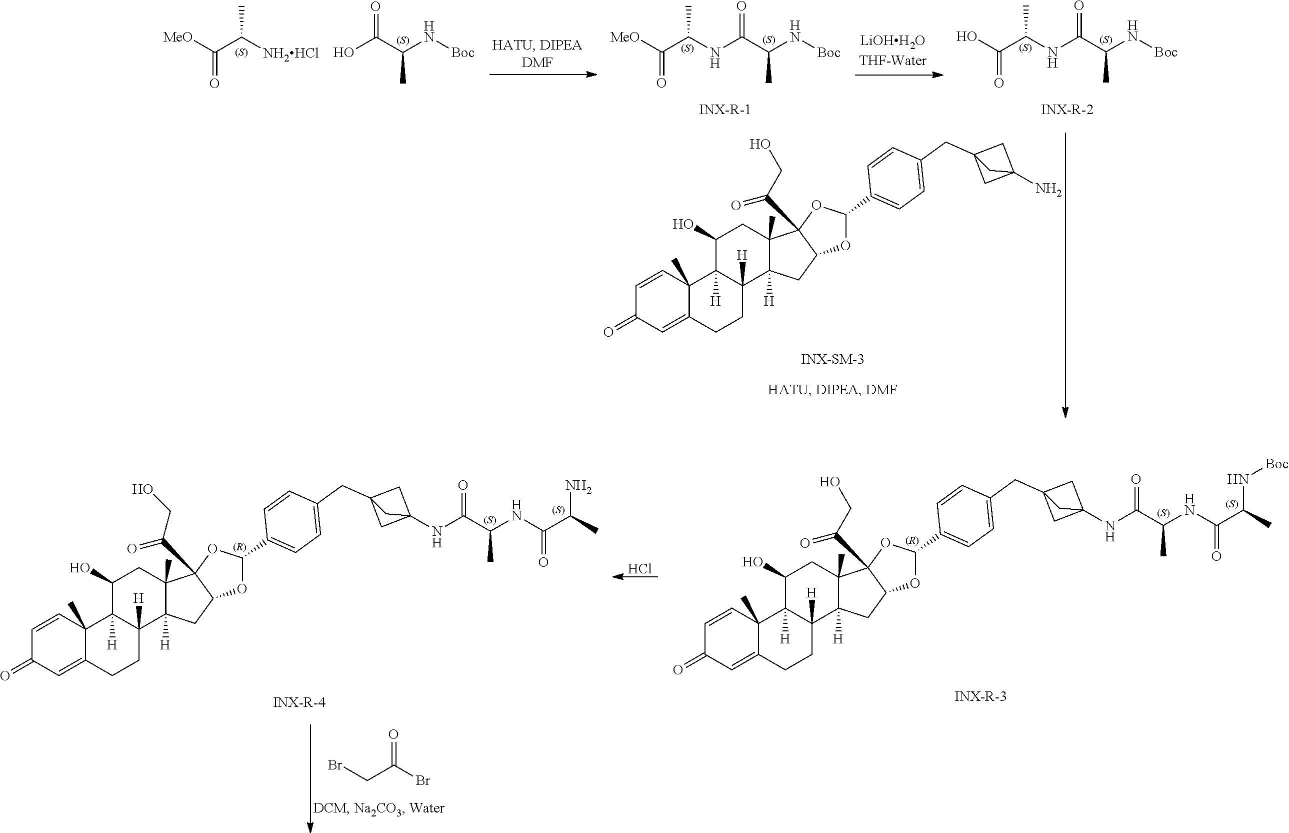

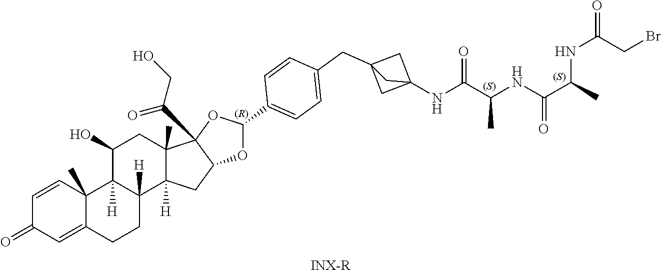

### Synthesis of methyl (tert-butoxycarbonyl)-L-alanyl-L-alaninate (INX-R-1)

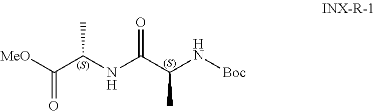

### Procedure

A 30 mL glass vial was charged with (tert-butoxycarbonyl)-L-alanine (5.0 g, 26.45 mmol), DIPEA (1.36 mL, 79.36 mmol) and DMF (50 mL) under nitrogen. To this solution, HATU (15.07 g, 39.67 mmol) was added at 0° C. followed by methyl L-alaninate hydrochloride (3.69 g, 26.45 mmol). Stirred the reaction mixture for 30 min at room temperature. After completion of reaction as indicated by TLC, reaction mixture was quenched with ice cold water and extracted with ethyl acetate. The combined organic layer was dried over Na₂SO₄ and evaporated under vacuum. The crude was triturated with hexane and DCM to give title compound as white solid (5.5 g, 56.20%). LCMS 275.3 (M+H)⁺.

### Synthesis of (tert-butoxycarbonyl)-L-alanyl-L-alanine (INX—R2)

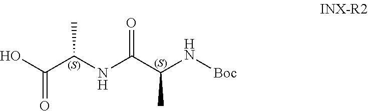

A 100 mL glass sealed vial was charged with (tert-butoxycarbonyl)-L-alanyl-L-alaninate (INX-R-1) (4.5 g, 16.42 mmol) and THF-Water (9:1) (55 mL). To this solution, LiOH·H₂O (20.69 g, 49.26 mmol) was added and stirred for 2 h at 60° C. After completion of reaction as indicated by TLC, reaction mixture was poured into water and extracted with ethyl acetate. The combined organic layer was dried over Na₂SO₄ and evaporated under vacuum. The crude was triturated with hexane and DCM to give title compound as white solid (4.0 g, 93.70%). LCMS: 261.20 (M+H)⁺.

### Synthesis of tert-butyl ((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-1-oxopropan-2-yl)amino)-1-oxopropan-2-yl)carbamate (INX-R-3)

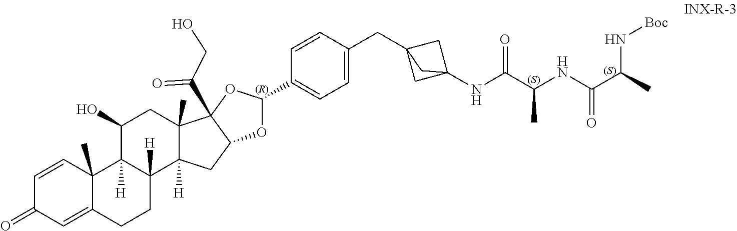

### Procedure

A 30 mL glass vial was charged with (tert-butoxycarbonyl)-L-alanyl-L-alanine (INX-R-2) (0.33 g, 1.26 mmol) in DMF (5 mL) and DIPEA (0.65 mL, 3.78 mmol) under nitrogen. To this solution, HATU (0.96 g, 2.52 mmol) Is added at 0° C. followed by (6aR,6bS,7S,8aS,8bS,10R,11aR,12aS,12bS)-10-(4-((3-aminobicyclo[1.1.1]pentan-1-yl)methyl)phenyl)-7-hydroxy-8b-(2-hydroxyacetyl)-6a,8a-dimethyl-1,2,6a,6b,7,8,8a,8b,11a, 12,12a,12b-dodecahydro-4H-naphtho[2′,1′:4,5]indeno[1,2-d][1,3]dioxol-4-one (INX-SM-3) (0.70 g, 1.26 mmol). The resulting reaction mixture was stirred for 1 h at room temperature. After completion of reaction as indicated by TLC, reaction mixture was quenched with ice cold water. The solid was filtered and dried under vacuum. The crude was purified by silica gel column chromatography (Methanol/DCM: 6:94) to give title compound as white solid (0.35 g, 34.42%). LCMS: 802.6 (M+H)⁺.

### Synthesis of (S)-2-amino-N—((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-1-oxopropan-2-yl) propanamide (INX-R-4)

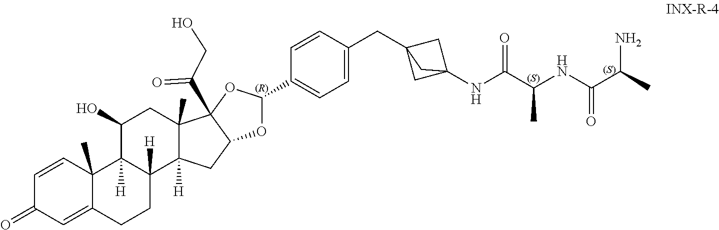

A 10 mL single-necked round bottom flask was charged with tert-butyl ((S)-1-(((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-1-oxopropan-2-yl)amino)-1-oxopropan-2-yl)carbamate (INX-R-3) (0.35 g, 0.44 mmol) and DCM (3 mL). To this solution, 2M HCl in diethyl ether (3 ml) was added and stirred for another 2 h at room temperature. After completion of reaction as indicated by TLC, reaction mixture was evaporated under vacuum and triturated with diethyl ether and n-pentane to give title compound as yellow solid (0.3 g, 97.92%). LCMS: 702.5 (M+H)⁺.

### Synthesis of (S)-2-(2-bromoacetamido)-N—((S)-1-((3-(4-((6aR,6bS,7S,8aS,8bS,10R,11aR, 12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-1-oxopropan-2-yl) propanamide (INX—R)

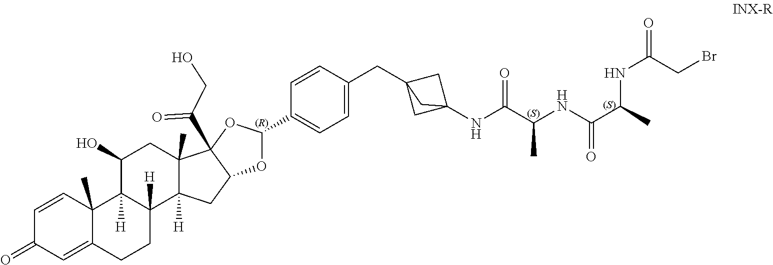

### Procedure

A 10 mL single-necked round bottom flask was charged with(S)-2-amino-N—((S)-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-1-oxopropan-2-yl) propanamide (INX-R-4) (0.30 g, 0.43 mmol) and DCM: Water (8:2) (3.6 mL) under nitrogen. To this solution, Na₂CO₃ (0.91 g, 0.855 mmol) was added followed by bromoacetyl bromine (0.87 g, 0.43 mmol) and stirred at room temperature for 1 h. After completion of reaction as indicated by TLC, reaction mixture was poured into water and extracted with DCM. The combined organic layer was dried over Na₂SO₄ and evaporated under vacuum. The crude title compound INX—R was purified by prep-HPLC (Column: Xbridge Prep, C18, OBD 19×250 mm, 5 μm, Mobile phase: A=0.05% NH₃ in water, B=acetonitrile; A:B, 65:35), Retention time 24.10 min to give R-Isomer as white solid (0.030 g, 8.53%); LCMS: 822.5, 824.4 (M&M+2); ¹H NMR (400 MHZ, MeOD, Key proton assignment): δ: 5.46 (s, 1H, Acetal-H), 5.05 (d, J=5.2 Hz, 1H, C16H).

### Synthesis of (S)-2-(2-(2-bromoacetamido)acetamido)-N1-(3-(4-((6aR,6bS,7S,8aS, 8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl) succinamide (INX—X)

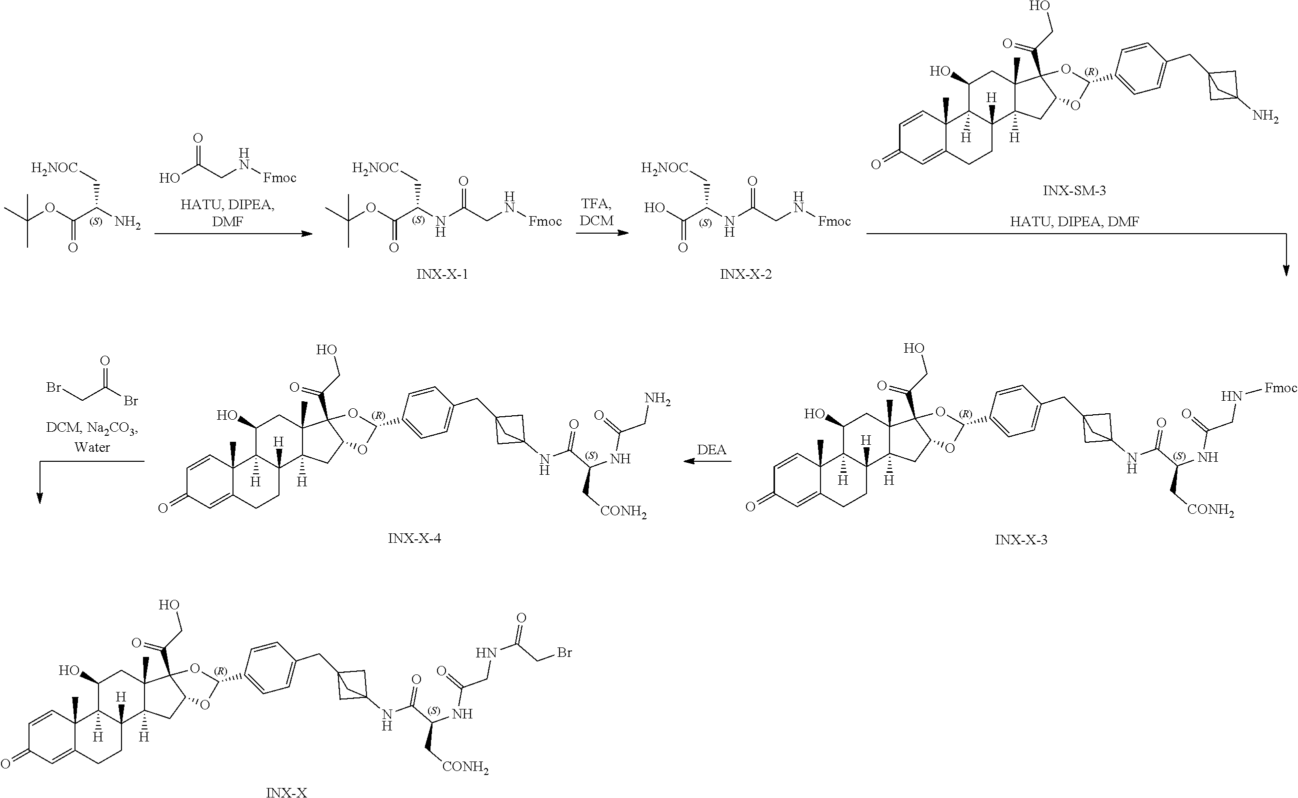

### Synthesis of methyl tert-butyl (((9H-fluoren-9-yl)methoxy)carbonyl) glycyl-L-asparaginate (INX—X-1)

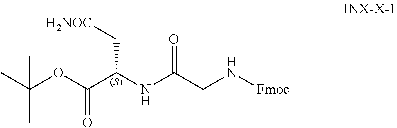

### Procedure

A 100 mL screw cap glass vial was charged with (((9H-fluoren-9-yl)methoxy)carbonyl) glycine (5.0 g, 16.83 mmol), DIPEA (8.68 mL, 50.50 mmol) and DMF (50 mL) under nitrogen. To this solution, HATU (7.67 g, 20.19 mmol) was added at 0° C. followed by tert-butyl L-asparaginate (3.79 g, 20.19 mmol). Stirred the reaction mixture for 1 hr at room temperature. After completion of reaction as indicated by TLC, reaction mixture was quenched with ice cold water and extracted with ethyl acetate. The combined organic layer was dried over Na₂SO₄ and evaporated under vacuum. The crude was triturated with hexane and DCM to give title compound as white solid (7.5 g, 95.41%). LCMS 412.83 (M-56).

### Synthesis of (((9H-fluoren-9-yl)methoxy)carbonyl)glycyl-L-asparagine (INX—X2)

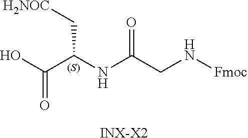

### Procedure

A 250 mL single-necked round bottom flask was charged with methyl tert-butyl (((9H-fluoren-9-yl)methoxy)carbonyl) glycyl-L-asparaginate (INX—X-1) (2.0 g, 4.28 mmol) and DCM (50 mL). To this solution, TFA (40 ml) was added and stirred for 2 h at room temperature. After completion of reaction as indicated by TLC, reaction mixture was evaporated under vacuum and triturated with diethyl ether and DCM to give title compound as white solid (1.5 g, 85.22%). LCMS: 412.8 (M+H)⁺.

### Synthesis of (9H-fluoren-9-yl) methyl (2-(((S)-4-amino-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-1,4-dioxobutan-2-yl)amino)-2-oxoethyl)carbamate (INX—X-3)

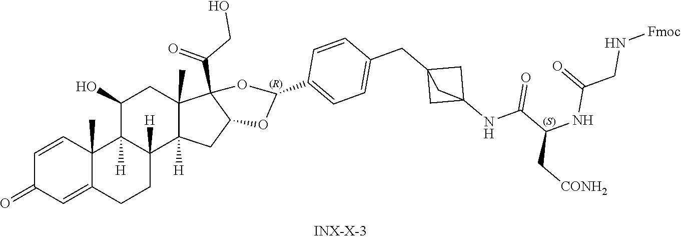

### Procedure

A 30 mL glass vial was charged with (((9H-fluoren-9-yl)methoxy)carbonyl) glycyl-L-asparagine (INX—X-2) (0.4 g, 0.973 mmol), DMF (5 mL), HATU (0.96 g, 2.52 mmol) and (6aR,6bS,7S,8aS,8bS,10R,11aR,12aS,12bS)-10-(4-((3-aminobicyclo[1.1.1]pentan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-3) (0.70 g, 1.26 mmol) under nitrogen. To this solution, DIPEA (0.50 mL, 2.91 mmol) was added and stirred for 30 min at room temperature. After completion of reaction as indicated by TLC, reaction mixture was quenched with ice cold water. The solid was filtered and dried under vacuum. The crude was triturated with diethyl ether and n-pentane to give title compound as white solid (0.6 g, 64.72%). LCMS: 954.24 (M+H)⁺.

### Synthesis of (S)-2-(2-aminoacetamido)-N1-(3-(4-((6aR,6bS,7S,8aS,8bS,10R,11aR,12aS, 12bS)-7-hydroxy-8b-(2-hydroxyacetyl)-6a,8a-dimethyl-4-oxo-2,4,6a,6b,7,8,8a,8b,11a,12, 12a,12b-dodecahydro-1H-naphtho[2′,1′:4,5]indeno[1,2-d][1,3]dioxol-10-yl)benzyl)bicycle[1.1.1]pentan-1-yl) succinamide (INX—X-4)

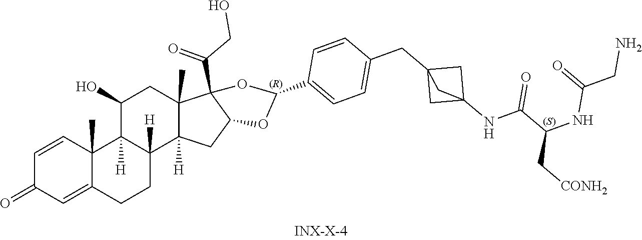

### Procedure

A 25 mL single-necked round bottom flask was charged with (9H-fluoren-9-yl) methyl (2-(((S)-4-amino-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-1,4-dioxobutan-2-yl)amino)-2-oxoethyl)carbamate (INX—X-3) (0.30 g, 0.314 mmol) and THF (5 mL). To this solution, DEA (0.48 mL, 4.72 mmol) was added and stirred for another 4 h at room temperature. After completion of reaction as indicated by TLC, reaction mixture was evaporated under vacuum and triturated with hexane to give title compound as yellow solid (0.20 g, 96.06%). LCMS: 731.0 (M+H)⁺.

### Synthesis of (S)-2-(2-(2-bromoacetamido)acetamido)-N1-(3-(4-((6aR,6bS,7S,8aS, 8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl) succinamide (INX—X)

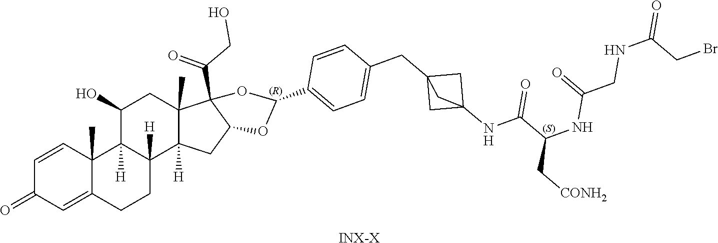

### Procedure

A 25 mL single-necked round bottom flask was charged with(S)-2-(2-aminoacetamido)-N1-(3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl) succinamide (INX—X-4) (0.20 g, 0.273 mmol) and THF-Water (8:2) (3.6 mL) under nitrogen. To this reaction mixture, Na₂CO₃ (0.58 g, 0.55 mmol) followed by bromoacetyl bromine (0.066 g, 0.33 mmol) was added and stirred for 4 h at rt. After completion of reaction as indicated by TLC, reaction mixture was poured into water and extracted with ethyl acetate. The combined organic layer was dried over Na₂SO₄ and evaporated under vacuum. The crude title compound INX—X was purified by prep-HPLC (Column: YMC-Actus Triart Prep C18-S, 250×20 mm S-5 μm, 12 nm, Mobile phase: A=0.05% NH₃ in water, B=acetonitrile; A:B 62:38), Retention time 17.79 min to give R-Isomer as white solid (0.030 g, 8.53%); LCMS: 852.7 (M+H)⁺; ¹H NMR (400 MHZ, MeOD, Key proton assignment): δ: 5.46 (s, 1H, Acetal-H), 5.06 (d, J=5.2 Hz, 1H, C16H).

Synthesis of ((6aS,6bR,7S,8aS,8bS,10R, (S)-4-(2-(2-bromoacetamido)acetamido)-5-((3-(4-11aR,12aS,12bS)-6b-fluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoic acid (INX—Y)

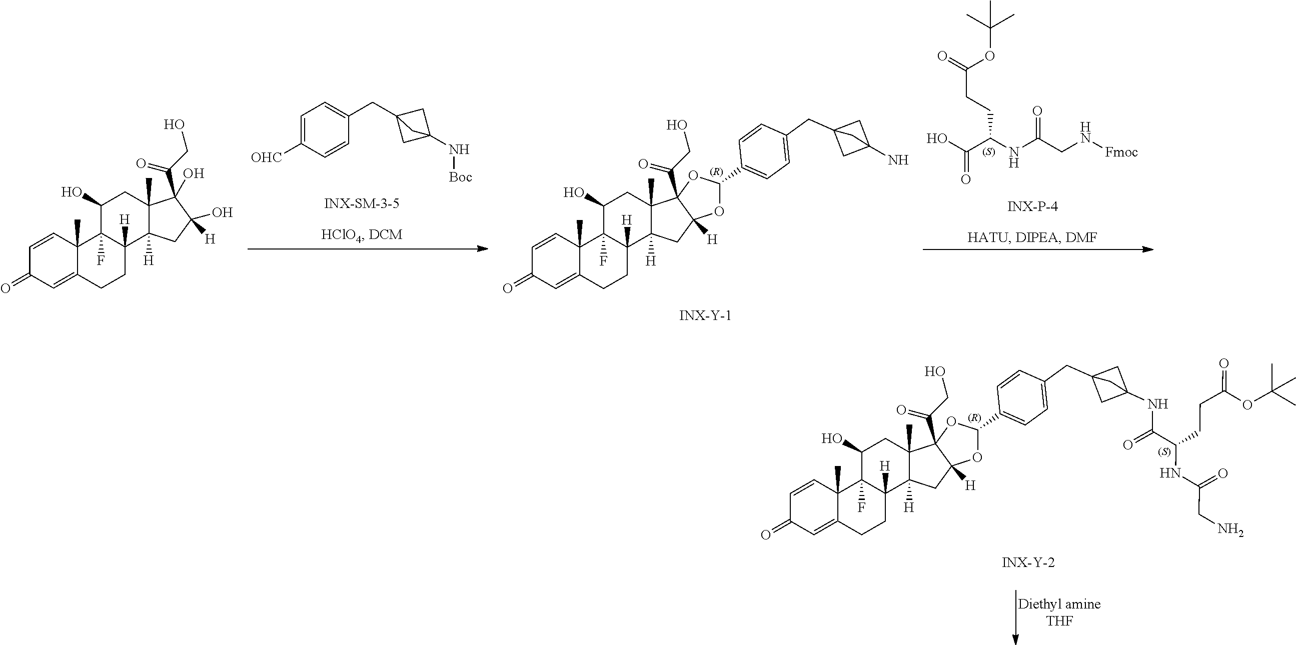

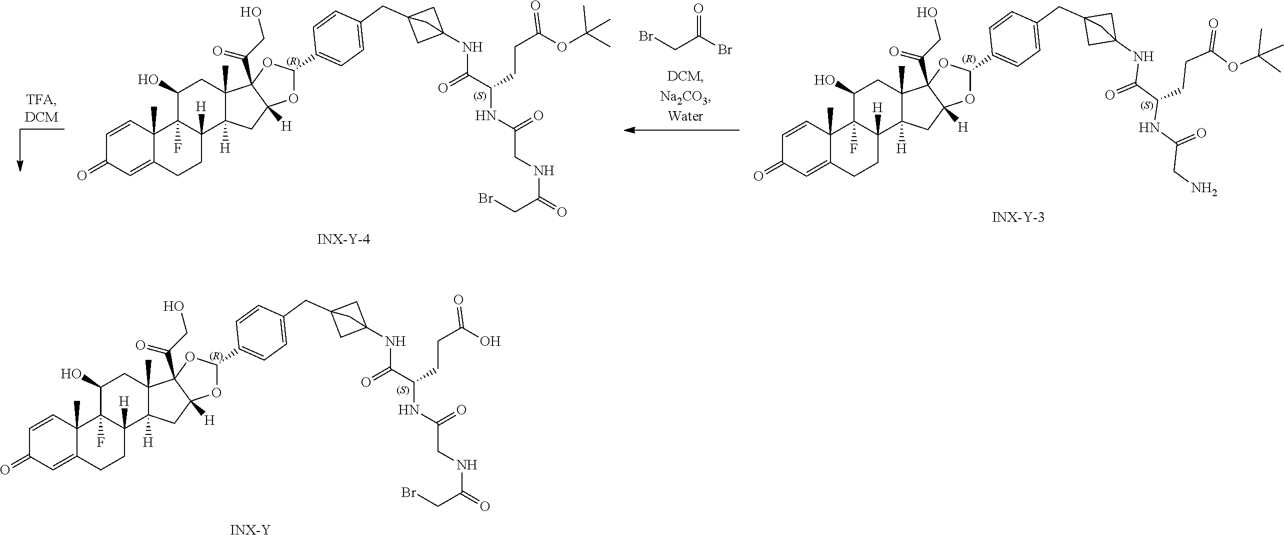

### Synthesis of (6aS,6bR,7S,8aS,8bS,10R,11aR,12aS,12bS)-10-(4-((3-aminobicyclo[1.1.1]pentan-1-yl)methyl)phenyl)-6b-fluoro-7-hydroxy-8b-(2-hydroxyacetyl)-6a,8a-dimethyl-1,2,6a,6b,7,8, 8a,8b,11a,12,12a,12b-dodecahydro-4H-naphtho[2′,1′:4,5]indeno[1,2-d][1,3]dioxol-4-one (INX—Y-1)

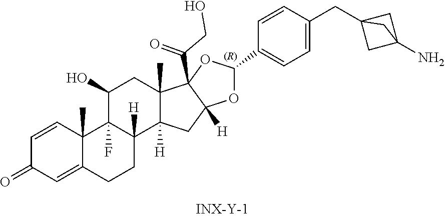

### Procedure

A 100 ml single-necked round bottom flask was charged with (8S,9R,10S,11S,13S,14S,16R,17S)-9-fluoro-11,16, 17-trihydroxy-17-(2-hydroxyacetyl)-10,13-dimethyl-6,7,8,9,10,11,12,13,14,15,16,17-dodecahydro-3H-cyclopenta[a]phenanthren-3-one (Triamcinolone) (1.0 g, 2.53 mmol) and tert-butyl (3-(4-formylbenzyl)bicyclo[1.1.1]pentan-1-yl)carbamate (INX-SM-3-5) (0.76 g, 2.53 mmol) and DCM (10 mL). To this solution, MgSO₄ (1.51 g, 12.65 mmol) was added and stirred for 5 min at room temperature. HClO₄ (1.2 g, 12.65 mmol) was added to the reaction mixture and stirred for another 1 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water; 60:40) to give title compound as light yellow (0.5 g, 34.14%). LCMS: 579.4 (M+H)⁺

### Synthesis of tert-butyl(S)-4-(2-((((9H-fluoren-9-yl)methoxy)carbonyl)amino)acetamido)-5-((3-(4-((6aS,6bR,7S,8aS,8bS,10R,11aR,12aS,12bS)-6b-fluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—Y-2)

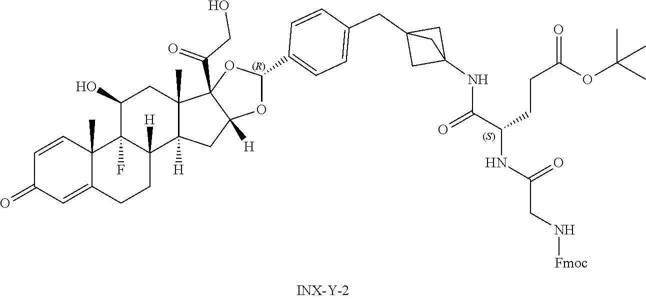

### Procedure

A 50 mL single-necked round bottom flask was charged with(S)-2-(2-((((9H-fluoren-9-yl)methoxy)carbonyl)amino)acetamido)-5-(tert-butoxy)-5-oxopentanoic acid (INX-P-4) (0.42 g, 0.87 mmol), HATU (0.49 g, 1.30 mmol), DMF (4 mL) and DIPEA (0.22 g, 1.74 mmol) at room temperature. To this solution, (6aS,6bR,7S,8aS,8bS,10R,11aR,12aS,12bS)-10-(4-((3-aminobicyclo[1.1.1]pentan-1-yl)methyl)phenyl)-6b-fluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX—Y-1) (0.50 g, 0.97 mmol) was added at room temperature and stirred for 1 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water, 50:50) to give title compound as light-yellow solid (0.65 g, 71.42%).

### Synthesis of tert-butyl(S)-4-(2-aminoacetamido)-5-((3-(4-((6aS,6bR,7S,8aS,8bS,10R,11aR,12aS,12bS)-6b-fluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—Y-3)

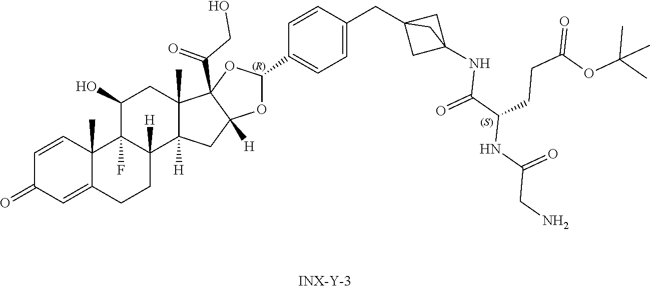

A 50 mL single-necked round bottom flask was charged with tert-butyl(S)-4-(2-((((9H-fluoren-9-yl)methoxy)carbonyl)amino)acetamido)-5-((3-(4-((6aS,6bR,7S,8aS,8bS,10R,11aR,12aS,12bS)-6b-fluoro-7-hydroxy-8b-(2-hydroxyacetyl)-6a,8a-dimethyl-4-oxo-2,4,6a,6b,7,8, 8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—Y-2) (0.65 g, 0.62 mmol) in THF (4 mL).To this solution, diethyl amine (0.40 g, 64.24 mmol) was added at room temperature and stirred for 3 h at room temperature. After completion of reaction as indicated by TLC, reaction mixture was evaporated under vacuum and triturated with diethyl ether to give title compound as yellow solid (0.42 g, 84.82%). LCMS: 821.4 (M+H)⁺.

### Synthesis of tert-butyl(S)-4-(2-(2-bromoacetamido)acetamido)-5-((3-(4-((6aS,6bR,7S,8aS,8bS,10R,11aR,12aS,12bS)-6b-fluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—Y-4)

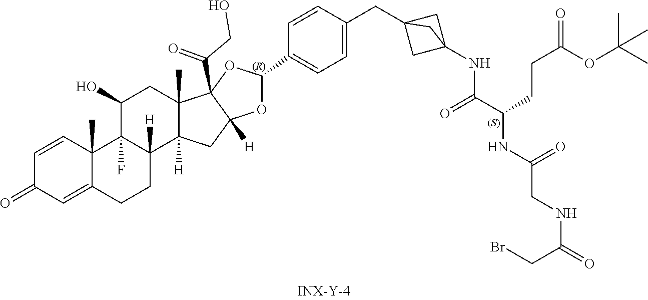

### Procedure

A 50 mL single-necked round bottom flask was charged with tert-butyl(S)-4-(2-aminoacetamido)-5-((3-(4-((6aS,6bR,7S,8aS,8bS,10R,11aR,12aS,12bS)-6b-fluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—Y-3) (0.42 g, 0.51 mmol) in DCM (10 mL). To this solution, Na₂CO₃ (0.11 g, 1.02 mmol) dissolved in water (1 ml) followed by bromoacetyl bromide (0.10 g, 0.51 mmol) were added at room temperature and stirred for 1 h. After completion of reaction as indicated by TLC, reaction mixture was quenched with water and extracted with DCM. The combined organic layer was dried over Na₂SO₄ and evaporated under vacuum. The crude was purified by reverse phase column chromatography (acetonitrile:water, 60:40) to give title compound as pale yellow solid (0.20 g, 41.45%). LCMS: 942.0 (M+H)⁺.

### Synthesis of (S)-4-(2-(2-bromoacetamido)acetamido)-5-((3-(4-((6aS,6bR,7S,8aS,8bS,10R,11aR,12aS,12bS)-6b-fluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoic acid (INX—Y)

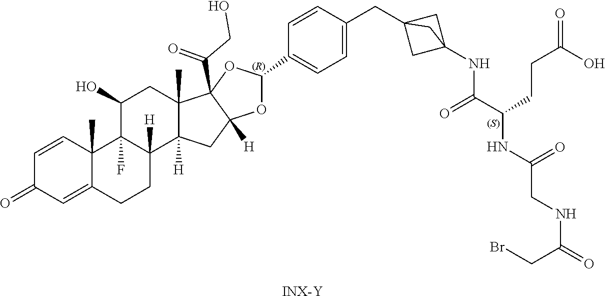

### Procedure

A 10 mL single-necked round bottom flask was charged with tert-butyl(S)-4-(2-(2-bromoacetamido)acetamido)-5-((3-(4-((6aS,6bR,7S,8aS,8bS,10R,11aR,12aS,12bS)-6b-fluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—Y-4) (0.20 g, 0.20 mmol) and DCM (2 mL). To this solution, TFA (0.11 g, 1.01 mmol) was added and stirred for 2 h at room temperature. After completion of reaction as indicated by TLC, reaction mixture was evaporated under vacuum. The crude INX—Y was purified by prep-HPLC (Column: Xbridge Prep, C18, 30×250 mm, 5 μm, Mobile phase: A=0.1% Formic acid in water, B=ACN: MeOH, 50:50; A:B, 47:53); Retention time 18.83 min to give R-Isomer (Fr-1) as white solid (0.040 g, 22.37%). LCMS: 885.8 (M+H)⁺; ¹H NMR (400 MHZ, DMSO-d6, Key proton assignment): δ: 5.48 (s, 1H, Acetal-H), 5.06 (d, J=4.8 Hz, 1H, C16H).

### Synthesis of (S)-4-(2-(2-bromoacetamido)acetamido)-5-((3-(4-((2S,6aS,6bR,7S,8aS,8bS, 10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoic acid (INX—S)

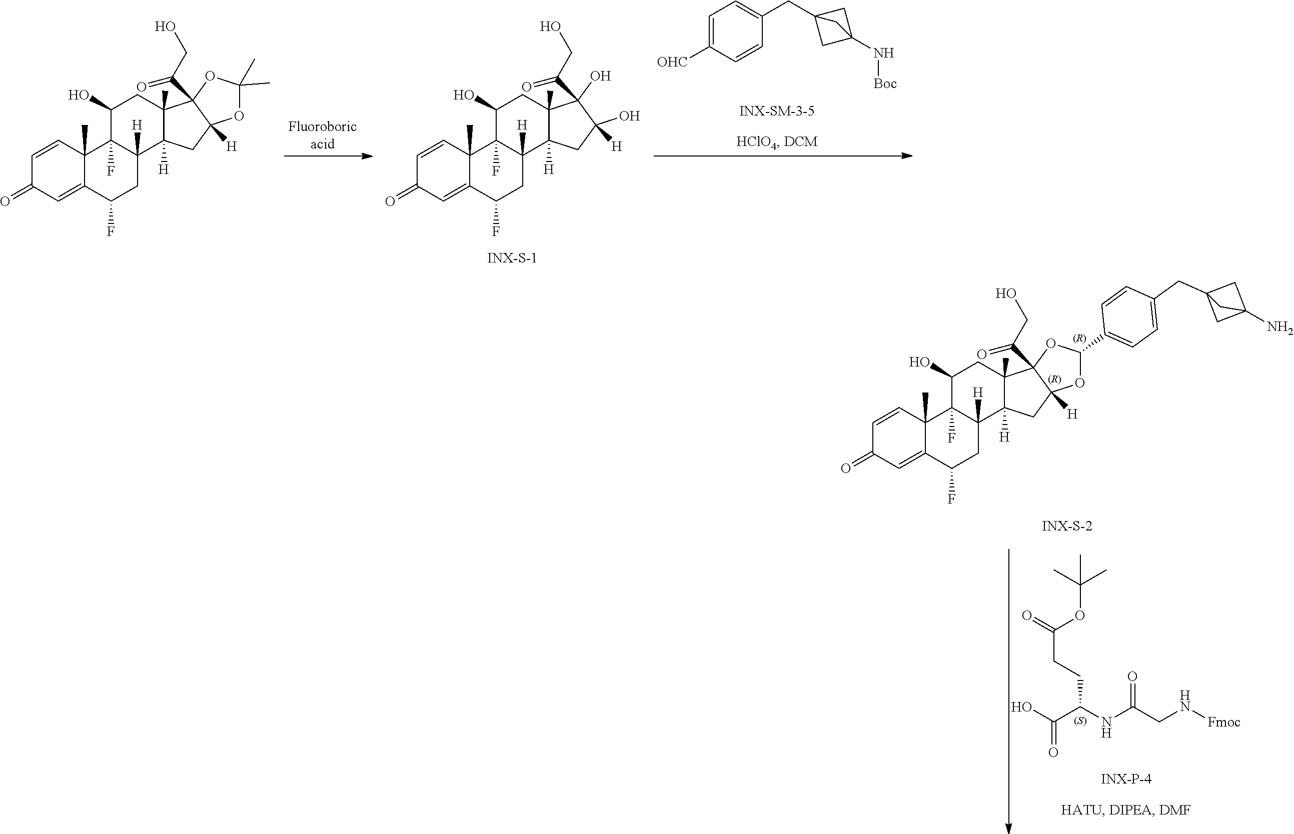

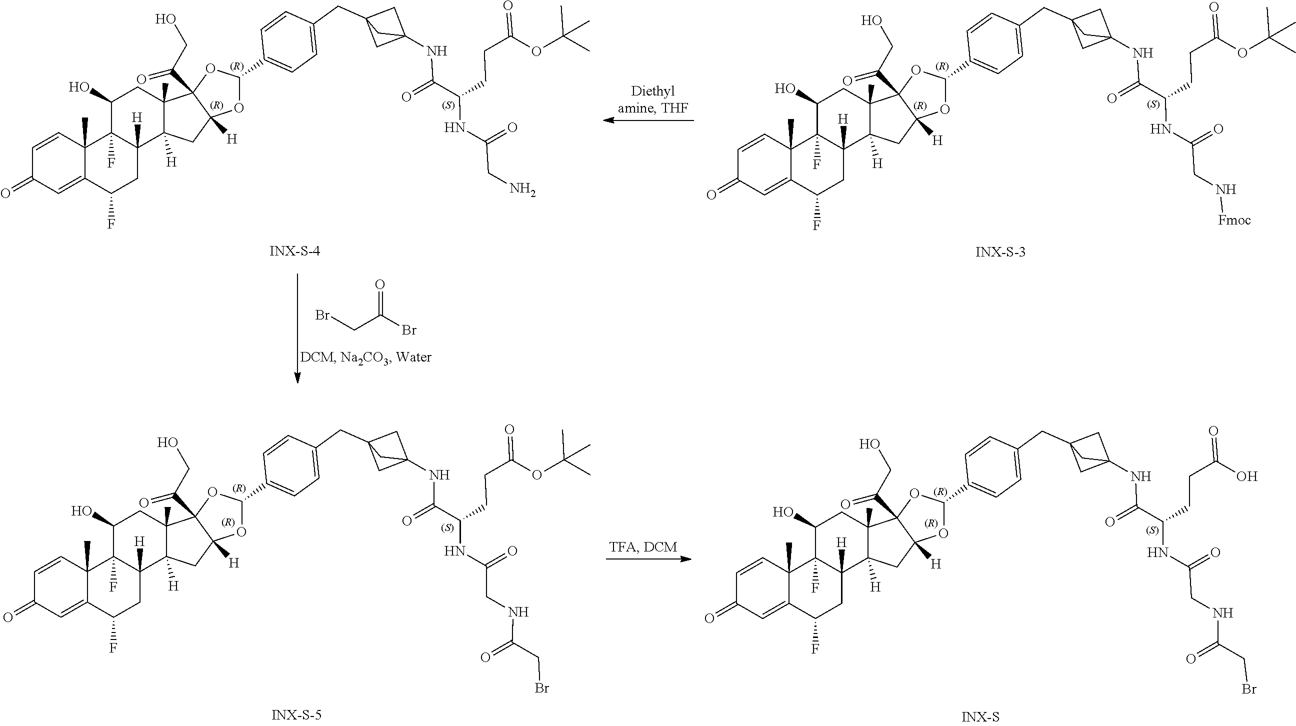

### Synthesis of (6S,8S,9R,10S,11S,13S,14S,16R,17S)-6,9-difluoro-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (INX—S-1)

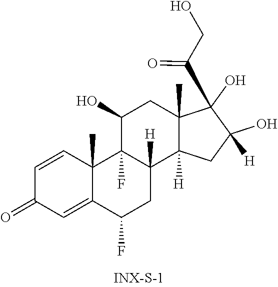

### Procedure

A 25 mL single-necked round bottom flask was charged with (2S,6aS,6bR,7S,8aS,8bS,11aR,12aS,12bS)-2,6b-difluoro-7-hydroxy-8b-(2-hydroxyacetyl)-6a,8a,10, 10-tetramethyl-1,2,6a,6b,7,8,8a,8b,11a,12,12a,12b-dodecahydro-4H-naphtho[2′,1′:4,5]indeno[1,2-d][1,3]dioxol-4-one (Fluocinolone acetonide) (1.0 g) and 50% aqueous HBF₄ (20 ml) was added and then stirred for another 16 h at room temperature. After completion of reaction as indicated by TLC, the solid was filtered, washed with water and dried under vacuum to yield INX—S-1 (1.0 g, quantitative). LCMS: 413.3 (M+H)⁺.

### Synthesis of ((2S,6aS,6bR,7S,8aS,8bS,10R,11aR,12aS,12bS)-10-(4-((3-aminobicyclo[1.1.1]pentan-1-yl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX—S-2)

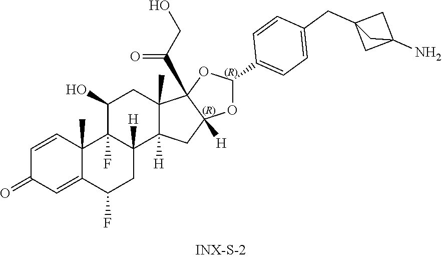

### Procedure

A 25 mL single-necked round bottom flask was charged with 6S,8S,9R,10S,11S,13S,14S,16R,17S)-6,9-difluoro-11,16,17-trihydroxy-17-(2-hydroxyacetyl)-10,13-dimethyl-6,7,8,9,10,11,12,13,14,15, 16, 17-dodecahydro-3H-cyclopenta[a]phenanthren-3-one (INX—S-1) (1.0 g, 2.42 mmol) and tert-butyl (3-(4-formylbenzyl)bicyclo[1.1.1]pentan-1-yl)carbamate (0.80 g, 2.66 mmol) and DCM (10 mL). To this solution, MgSO₄ (1.42 g, 12.14 mmol) was added and stirred for another 5 min at room temperature. HClO₄ (1.2 g, 12.14 mmol) was added and stirred for another 1 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water, 60:40) to give title compound as light yellow (0.61 g, 42.28%). LCMS: 596.4 (M+H)⁺.

### Synthesis of tert-butyl(S)-4-(2-((((9H-fluoren-9-yl)methoxy)carbonyl)amino)acetamido)-5-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—S-3)

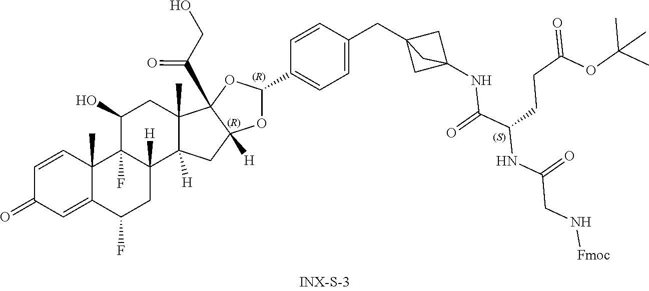

### Procedure

A 50 mL single-necked round bottom flask was charged with(S)-2-(2-((((9H-fluoren-9-yl)methoxy)carbonyl)amino)acetamido)-5-(tert-butoxy)-5-oxopentanoic acid (INX-P-4) (0.45 g, 0.93 mmol), HATU (0.53 g, 1.40 mmol), DMF (4 mL) and DIPEA (0.23 g, 1.86 mmol) at room temperature. To this solution, ((2S,6aS,6bR,7S,8aS,8bS,10R,11aR,12aS,12bS)-10-(4-((3-aminobicyclo[1.1.1]pentan-1-yl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX—S-2) (0.61 g, 1.02 mmol) was added at room temperature and stirred for 1 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water, 50:50) to give title compound as light-yellow solid (0.40 g, 56.19%). LCMS: 1061.5 (M+H)⁺.

### Synthesis of tert-butyl(S)-4-(2-aminoacetamido)-5-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—S-4)

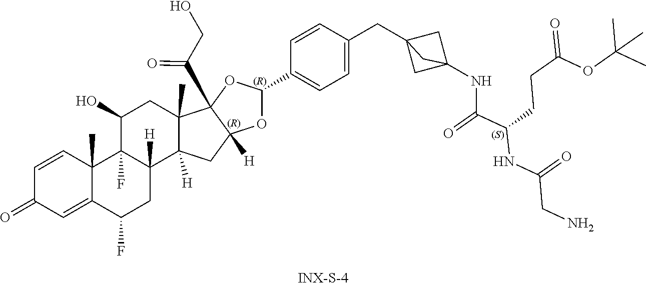

### Procedure

A 50 mL single-necked round bottom flask was charged with tert-butyl(S)-4-(2-((((9H-fluoren-9-yl)methoxy)carbonyl)amino)acetamido)-5-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—S3) (0.41 g, 0.39 mmol) and THF (4 mL). To this solution, diethyl amine (0.28 g, 3.91 mmol) was added at room temperature and stirred for 3 h. After completion of reaction as indicated by TLC, reaction mixture was evaporated under vacuum to give title compound as yellow solid (0.26 g, 82.24%). LCMS: 838.5 (M+H)⁺.

### Synthesis of tert-butyl(S)-4-(2-(2-bromoacetamido)acetamido)-5-((3-(4-((2S,6aS,6bR, 7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—S-5)

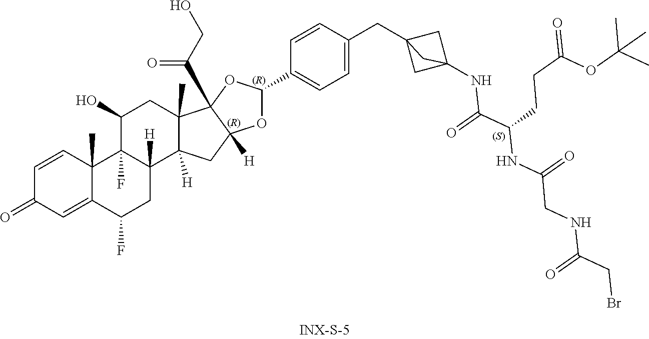

### Procedure

A 10 mL single-necked round bottom flask was charged with tert-butyl(S)-4-(2-aminoacetamido)-5-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—S4) (0.22 g, 0.26 mmol) in DCM (2 mL). To this solution, Na₂CO₃ (0.10 g, 0.53 mmol) in water (1 mL) followed by bromoacetyl bromide (0.030 g, 0.28 mmol) was added at room temperature and stirred for 1 h. After completion of reaction as indicated by TLC, reaction mixture was quenched with water and extracted with DCM. The combined organic layer was dried over Na₂SO₄ and evaporated under vacuum. The crude was purified by reverse phase column chromatography (acetonitrile/water: 60:40) to give title compound as light yellow (0.1 g, 39.72%). LCMS: 960.4 (M+H)⁺.

### Synthesis of (S)-4-(2-(2-bromoacetamido)acetamido)-5-((3-(4-((2S,6aS,6bR,7S,8aS,8bS, 10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoic acid (INX—S)

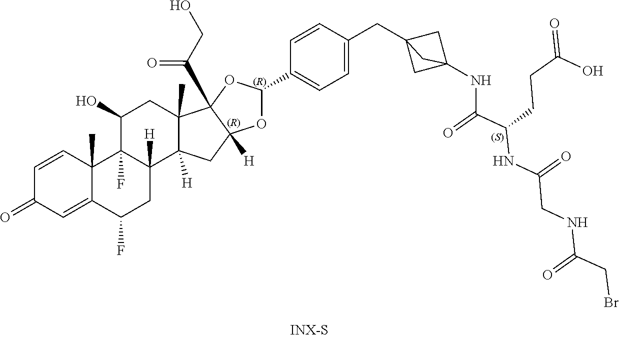

### Procedure

A 10 mL single-necked round bottom flask was charged with tert-butyl(S)-4-(2-(2-bromo acetamido)acetamido)-5-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicycle[1.1.1]pentan-1-yl)amino)-5-oxopentanoate (INX—S-5) (0.10 g, 0.10 mmol) in DCM (2 mL). To this solution, TFA (0.059 g, 0.52 mmol) was added at room temperature and stirred for 2 h at room temperature. After completion of reaction as indicated by TLC, reaction mixture was directly evaporated under vacuum. The crude INX—S was purified by prep-HPLC (Column: SUNFIRE Prep C18 OBD, 19×250 mm, 5 μm, Mobile phase: A=0.1% Formic acid in water, B=Acetonitrile; A:B, 65:35); Retention time 17.7 min to give R-Isomer as white solid (0.011 g, 11.68%). LCMS: 902.3, 904.3 (M & M+2). ¹H NMR (400 MHz, DMSO-d6, Key proton assignment): δ: 5.45 (s, 1H, Acetal-H), 4.95 (d, J=4.8 Hz, 1H, C16H).

### Synthesis of (S)-4-(2-(2-bromoacetamido)acetamido)-5-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoic acid (INX-T)

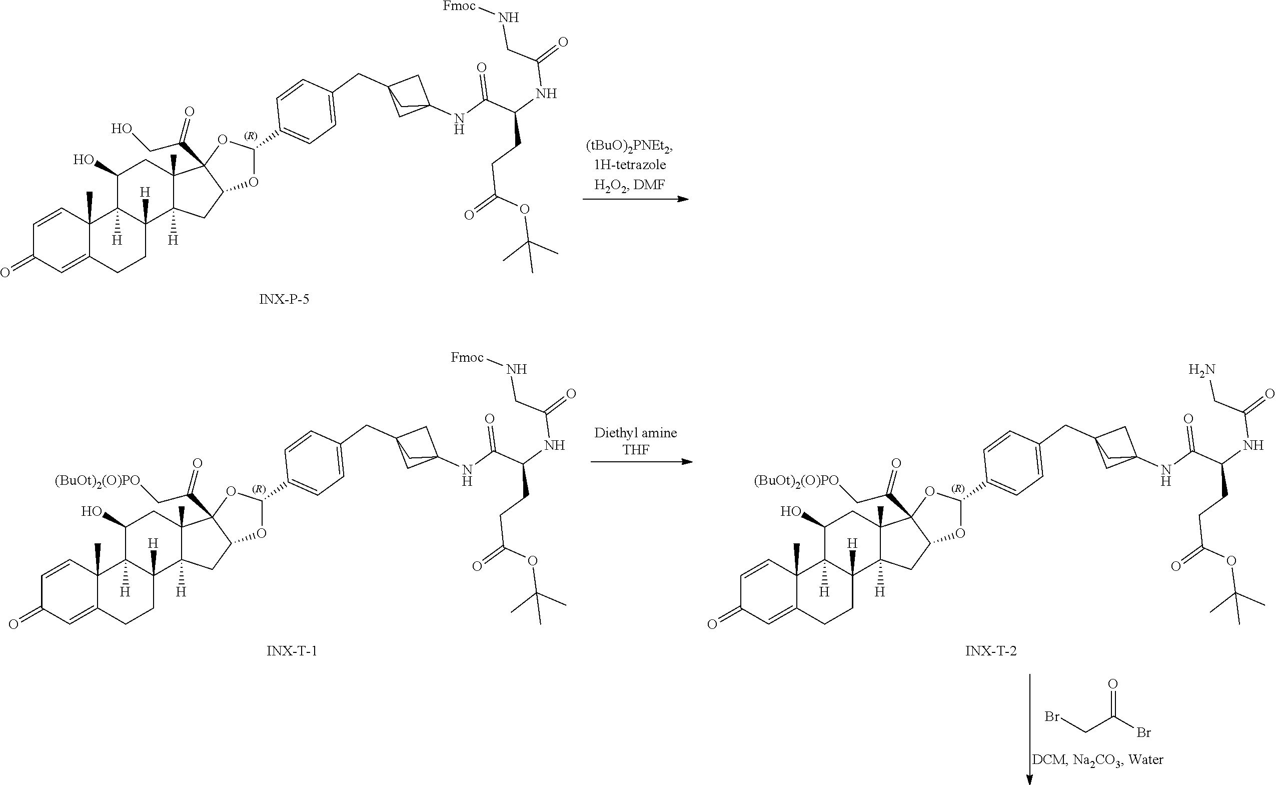

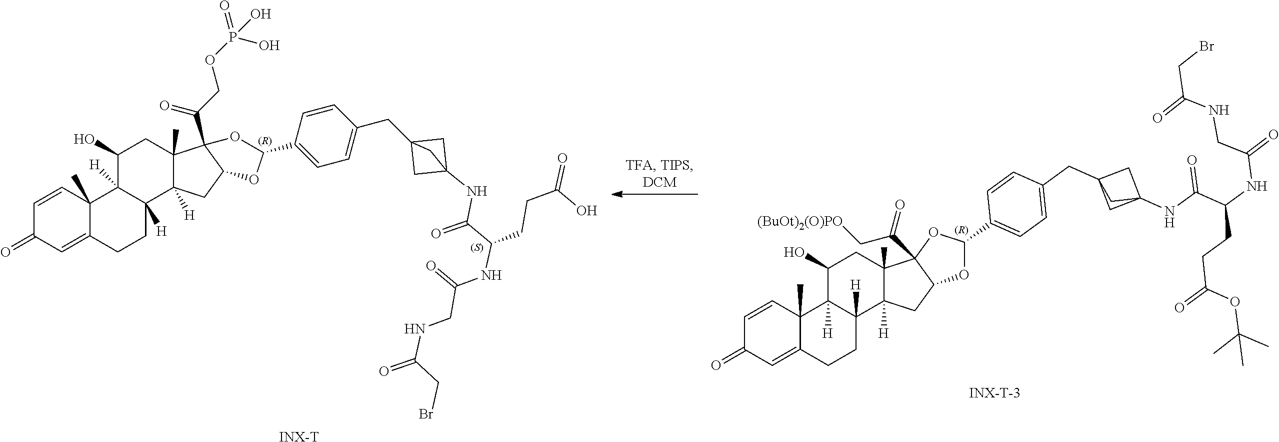

### Synthesis of tert-butyl(S)-4-(2-((((9H-fluoren-9-yl)methoxy)carbonyl)amino)acetamido)-5-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-T-1)

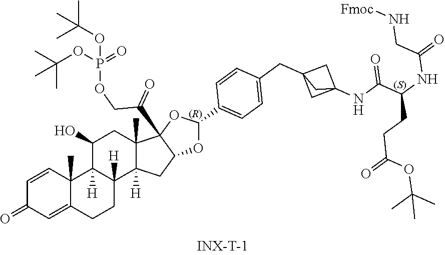

A 10 mL vial was charged with tert-butyl(S)-4-(2-((((9H-fluoren-9-yl)methoxy)carbonyl)amino)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-P-5) (0.20 g, 0.19 mmol) and DMF (1 mL). To this solution, 1H-tetrazole (0.137 g, 1.950 mmol) and (tBuO)₂PNEt₂ (1.3 g, 4.68 mmol) were added at room temperature and stirred for 79 h at room temperature. After completion of reaction as indicated by TLC, hydrogen peroxide (1.3 g, 4.68 mmol) was added into the solution. The crude was purified by reverse phase column chromatography (acetonitrile:water, 80:20) to give title compound as light yellow solid (0.070 g, 29.47%). It was immediately used for next step.

### Synthesis of tert-butyl(S)-4-(2-aminoacetamido)-5-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl) amino)-5-oxopentanoate (INX-T-2)

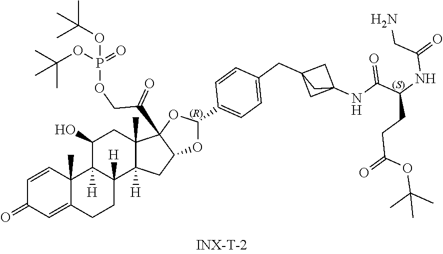

### Procedure

A 10 mL glass vial was charged with tert-butyl(S)-4-(2-((((9H-fluoren-9-yl)methoxy)carbonyl)amino)acetamido)-5-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-T-1) (0.070 g, 0.057 mmol) in THF (1 mL). To this solution, diethyl amine (0.042 g, 0.57 mmol) was added at room temperature and stirred for 16 h at room temperature. After completion of reaction as indicated by TLC, reaction mixture was concentrated. The crude was purified by trituration with diethyl ether and hexane to give title compound as pale yellow solid (0.30 g, 52.44%). It was used immediately for next step.

### Synthesis of tert-butyl(S)-4-(2-(2-bromoacetamido)acetamido)-5-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate

### (INX-T-3)

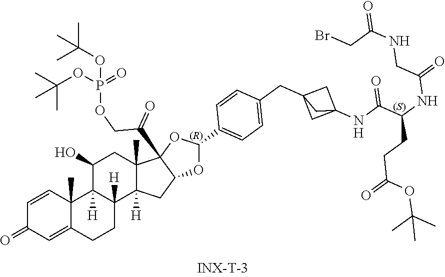

### Procedure

A 10 mL glass vial was charged with tert-butyl(S)-4-(2-aminoacetamido)-5-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-T-2) (0.030 g, 0.030 mmol) in DCM (1 mL). To this solution, Na₂CO₃ (0.006 g, 0.060 mmol) solution in water (0.1 mL) and bromoacetyl bromide (0.006 g, 0.030 mmol) were added stirred for 1 h at room temperature. After completion of reaction as indicated by TLC, reaction mixture was quenched with water and extracted with DCM. The combined organic layer was dried over Na₂SO₄ and evaporated under vacuum to give crude product as off white solid (0.040 g, crude)

### Synthesis of (S)-4-(2-(2-bromoacetamido)acetamido)-5-((3-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoic acid (INX-T)

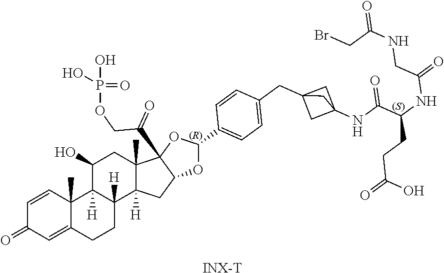

### Procedure

A 10 mL single-necked round bottom flask was charged with tert-butyl(S)-4-(2-(2-bromoacetamido)acetamido)-5-((3-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-T-3) (0.001 g, 0.0089 mmol) in DCM (1 mL). To this solution, TFA (0.005 g, 0.043 mmol) and catalytic triisopropylsilane were added at room temperature and stirred for 20 min. After completion of reaction as indicated by TLC, reaction mixture was evaporated under vacuum to give title compound as yellow solid. (0.006 g, 71%). LCMS: 946.2, 948.2 (M& M+2).

### Synthesis of INX-A

### Reaction Scheme

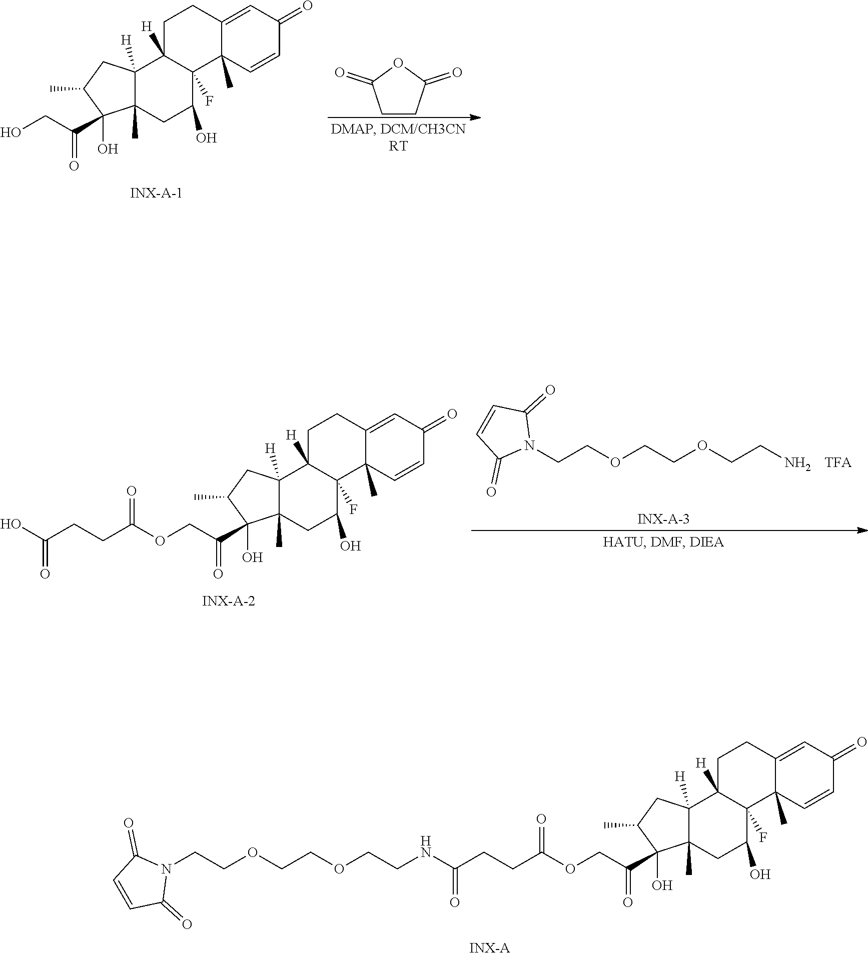

### Synthesis of INX-A-2

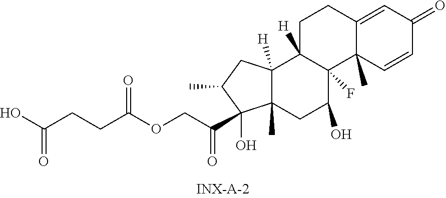 [SMILES: C[C@@H]1C[C@H]2[C@@H]3CCC4=CC(=O)C=C[C@]4(C)[C@@]3(F)[C@@H](O)C[C@]2(C)[C@@]1(O)C(=O)COC(=O)CCC(=O)O]

### Procedure

To a solution of compound INX-A-1 (3.0 g, 7.64 mmol, 1.0 eq) in a dichloromethane/acetonitrile (500 mL/100 mL) were added cyclic anhydride (3.0 g, 30.58 mmol, 4.0 eq) and DMAP (1.8 g, 15.29 mmol, 2.0 eq). The reaction mixture was allowed to stir at rt for 2 h and the mixture was concentrated under reduced pressure. The residue was purified by column chromatography on silica gel eluted with DCM/MeOH (10% to 15%)+0.1% AcOH to afford the compound INX-A-2 (3.2 g, 85%) as white solid. TLC: DCM/MeOH=10:1. Rf (Compound 1)=0.45. Rf (Compound 2)=0.30. LC-MS: (M+H)+=394.40

### Synthesis of INX-A

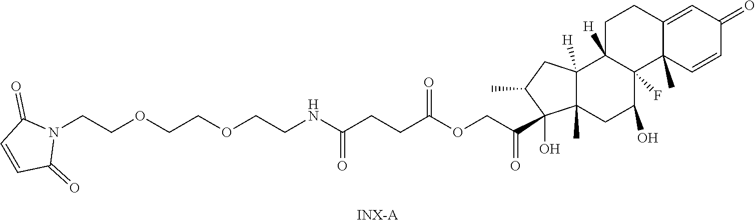

### Procedure

To a solution of INX-A-2 (220 mg, 0.45 mmol) and INX-A-3 (230 mg, 0.67 mmol) in NMP (4 mL) was added HATU (342 mg, 0.90 mmol) and DIPEA (232 mg, 1.8 mmol). The mixture was stirred at rt for 5 h. The mixture was purified by prep-HPLC (ACN/H₂O, 0.1% HCOOH) to give INX-A (122 mg, 39%). LCMS: [M+H]⁺=703; ¹H NMR (CDCl₃, 300 MHZ) (δ, ppm) 7.20 (d, J=9.0 Hz, 1H), 6.73 (s, 2H), 6.52 (br, 1H), 6.33 (d, J=9.0 Hz, 1H), 6.11 (s, 1H), 4.91 (q, J=17.3 Hz, 2H), 4.35 (d=9.3 Hz, 1H), 3.76-3.42 (m, 10H), 3.03 (m, 1H), 2.79 (m, 2H), 2.65-2.56 (m, 3H), 2.42-2.06 (m, 7H), 1.84-1.63 (m, 3H), 1.22 (m, 1H), 1.02 (s, 3H), 0.90 (d, J=7.2 Hz, 3H). 19F NMR (CDCl₃) (δ, ppm)-166.09 (q).

All reactions described below were conducted under a dry nitrogen atmosphere unless otherwise stated. All the key chemicals were used as received. All other commercially available materials, such as solvents, reagents and catalyst were used without further purification. Reactions were monitored by thin layer chromatography (TLC) using pre-coated Merck silica gel 60F254 aluminium sheets (Merck, Germany). The visualization of TLC plates was accomplished using the UV light, ninhydrine spray, and iodine vapours. ¹H NMR (400 MHZ) was recorded on Bruker Advancer-III HD FT-NMR spectrophotometer (Bruker, USA) and interpreted manually. Column chromatographic separations were carried out using 230-400 mesh, 100-200 mesh and 60-120 mesh silica gel or C18 silica as stationary phase using appropriate mobile phase.

### Following are the LCMS Methods Used for the Analysis of Final Targets.

### LCMS Method-1

Column Details: X-BRIDGE BEH 2.1*50 mm 2.5 μm

Machine Details:—Water Acquity UPLC-H Class equipped with PDA and Acquity SQ detector, Column temperature: 35° C., Auto sampler temperature: 5° C., Mobile Phase A: 0.1% Formic acid in Milli Q water (pH=2.70), Mobile Phase B: 0.1% Formic acid in Milli Q water: Acetonitrile (10:90).

Mobile phase gradient details: T=0 min (97% A, 3% B) flow: 0.8 mL/min; T=0.75 min (97% A, 3% B) flow: 0.8 mL/min; gradient to T=2.7 min (2% A, 98% B) flow: 0.8 mL/min; gradient to T=3 min (0% A, 100% B) flow: 1 mL/min; T=3.5 min (0% A, 100% B) flow: 1 mL/min; gradient to T=3.51 min (97% A, 3% B) flow: 0.8 mL/min; end of run at T=4 min (97% A, 3% B), Flow rate: 0.8 mL/min, Flow rate: 0.8 mL/min, Run Time: 4 min, UV Detection Method: PDA.

### Mass Parameter:

Probe: ESI, Mode of Ionisation: positive and negative, Cone voltage: 30V and 10 V, capillary voltage: 3.0 KV, Extractor Voltage: 1V, Rf Lens: 0.1 V, Temperature of source: 120° C., Temperature of Desolvation: 400° C. Cone Gas Flow: 100 L/hour, Desolvation Gas flow: 800 L/hour.

### LCMS Method-2

Column Details: X-BRIDGE BEH 2.1*50 mm 2.5 μm

Machine Details: Waters Acquity Ultra performance LC equipped with PDA and attached with QDA detector, Column temperature: 35° C., Auto sampler temperature: 5° C., Mobile Phase A: 0.1% Formic acid in Milli Q water (pH=2.70), Mobile Phase B: 0.1% Formic acid in Milli Q water: Acetonitrile (10:90). Mobile phase gradient details: T=0 min (97% A, 3% B) flow: 0.8 mL/min; T=0.75 min (97% A, 3% B) flow: 0.8 mL/min; gradient to T=2.7 min (2% A, 98% B) flow: 0.8 mL/min; gradient to T=3 min (0% A, 100% B) flow: 1 mL/min; T=3.5 min (0% A, 100% B) flow: 1 mL/min; gradient to T=3.51 min (97% A, 3% B) flow: 0.8 mL/min; end of run at T=4 min (97% A, 3% B), Flow rate: 0.8 mL/min, Run Time: 4 min, UV Detection Method: PDA.

Mass Parameter: Probe: ESI, Mode of Ionisation: Positive and Negative, Cone voltage: 30 V and 10 V, capillary voltage: 0.8 KV, Extractor Voltage: 1V, Rf Lens: 0.1 V, Temperature of source: 120° C., Temperature of Probe: 600° C. Cone Gas Flow: Default, Desolvation Gas flow: Default.

### LCMS Method-3

Column Details: Xtimate C18 4.6*50 mm 5 μm

Machine Details:—Waters Acquity Ultra performance LC connected with PDA and equipped with SQ detector, Column temperature: 35° C., Auto sampler temperature: 5° C., Mobile Phase A: 5 mM Ammonium Bicarbonate (pH=7.35) in Milli Q water, Mobile Phase B: Acetonitrile. Mobile phase gradient details: T=0 min (97% A, 3% B) Flow rate=1.0 ml/min, T=0.20 min (97% A, 3% B) Flow rate=1.0 ml/min; gradient to T=2.70 min (20% A, 80% B) Flow rate=1.0 ml/min; gradient to T=3.0 min (0% A, 100% B) Flow rate=1.2 ml/min; T=3.50 min (0% A, 100% B) Flow rate=1.2 ml/min; T=3.51 min (97% A,3% B) Flow rate=1.0 ml/min; end of run at T=4.0 min (97% A, 3% B) Flow rate=1.0 ml/min, Run Time: 4 min, UV Detection Method: PDA.

Mass Parameter: Probe: ESI, Mode of Ionisation: Positive and Negative, Cone voltage: 30 and 10 V, capillary voltage: 3.0 KV, Extractor Voltage: 2 V, Rf Lens: 0.1 V, Temperature of source: 120° C., Temperature of Probe: 400° C., Cone Gas Flow: 100 L/Hr, Desolvation Gas flow: 800 L/Hr.

### LCMS Method-4

Column Details: Xtimate C18 4.6*150 mm 5 μm

Machine Details: Waters 996 Photodiode Array Detector equipped with Waters Micro mass ZQ detector, Column temperature: 35° C., Auto sampler temperature: 15° C., Mobile Phase A: 5 mM Ammonium Acetate and 0.1% Formic acid (pH=3.50) in Milli Q water, Mobile Phase B: Methanol

Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.0 mL/min, Run Time: 17 min, UV Detection Method: PDA.

### Mass Parameter:

Probe: ESI, Mode of Ionisation: Positive and Negative, Cone voltage: 30 and 10V, capillary voltage: 3.0 KV, Extractor Voltage: 2V, Rf Lens: 0.1V, Temperature of source: 120° C., Temperature of Probe: 400° C., Cone Gas Flow: 100 L/Hr, Desolvation Gas flow: 800 L/Hr.

### LCMS Method-5

Column Details: Sunfire C18 150×4.6 mm, 3.5 μm
Machine Details: Agilent 1260 Infinity-II and G6125C (LC/MSD) mass detector, Column temperature: 35° C., Auto sampler temperature: 15° C., Mobile Phase A: 5 mM Ammonium Acetate and 0.1% Formic acid (pH=3.50) in Milli Q water, Mobile Phase B: Methanol
Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.0 mL/min, Run Time: 17 min, UV Detection Method: PDA.

### Mass Parameter:

Probe: MMI, Mode of Ionisation: (ESI) positive and negative, Fragment voltage: 30V & 70 V, capillary voltage: 3000 V, Gas temperature of source: 325° C., Temperature of vaporizer: 225° C., Gas Flow: 12 L/min, Nebulizer: 50.

### HPLC Method-1

Column Details: Sunfire C18 (150 mm×4.6 mm),3.5 μm

Machine Details: Agilent Technologies. 1260 Series, Infinity-II with PDA detector, Column temperature: 35° C., Auto sampler temperature: 15° C., Mobile Phase A: 0.05% Trifluoroacetic acid in Milli Q water (pH=2.2), Mobile Phase B: Acetonitrile.

Mobile phase gradient details: T=0 min (90% A, 10% B) flow: 1.0 mL/min; T=7.0 min (10% A, 90% B) flow: 1.0 mL/min; gradient to T=9.0 min (0% A, 100% B) flow: 1.0 mL/min; gradient to T=14 min (0% A, 100% B) flow: 1.0 mL/min; T=14.01 min (90% A, 10% B) flow: 1 mL/min; end of run at T=17 min (90% A, 10% B) flow: 1.0 mL/min, Flow rate: 1.0 mL/min, Run Time: 17 min, UV Detection Method: PDA.

### HPLC Method-2

Column Details: Atlantis C18 (150 mm×4.6 mm),5.0 μm or Welch C18 (150 mm 4.6 mm),5.0 μm

Machine Details:—Waters Alliance e2695 with 2998 PDA detector, Column temperature: 35° C., Auto sampler temperature: 15° C., Mobile Phase A: 0.1% Ammonia in Milli Q water (pH=10.5), Mobile Phase B: Acetonitrile. Mobile phase gradient details: T=0 min (90% A, 10% B) flow: 1.0 mL/min; T=7.0 min (10% A, 90% B) flow: 1.0 mL/min; gradient to T=9.0 min (0% A, 100% B) flow: 1.0 mL/min; gradient to T=14 min (0% A, 100% B) flow: 1.0 mL/min; T=14.01 min (90% A, 10% B) flow: 1 mL/min; end of run at T=17 min (90% A, 10% B) flow: 1.0 mL/min, Flow rate: 1.0 mL/min, Run Time: 17 min, UV Detection Method: PDA.

HPLC details: Waters Alliance e2695 with 2998 PDA detector; Column Details: Atlantis C18 (150 mm×4.6 mm),5.0 μm or Welch C18 (150 mm×4.6 mm), 5.0 μm; Mobile Phase A: 0.1% Ammonia in Milli Q water (pH=10.5), Mobile Phase B: Acetonitrile; Flow rate: 1.0 mL/min, Run Time: 17 min

### HPLC Method-3

Column Details: Agilent 1260 Infinity-II and G6125C (LC/MSD) mass detector, Column temperature: 35° C., Auto sampler temperature: 15° C., Mobile Phase A: 5 mM ammonium acetate+0.1% formic acid in water (pH=3.5) Mobile Phase B: MeOH. Mobile phase gradient details: T=0 minutes (90% A, 10% B); T=7.0 minutes (10% A, 90% B); gradient to T=9 minutes (0% A, 100% B gradient to T=14 minutes (0% A, 100% B); gradient to T=14.1 minutes (90% A, 10% B); T=17.0 minutes (90% A, 10% B). Flow rate: 1 mL/min, Runtime: 17 min. UV Detection Method: PDA. Mass parameter: Probe: MMI, Mode of Ionisation: (ESI) positive and negative, Fragment voltage: 70 V and 30V, capillary voltage: 3000 V, Gas temperature of source: 325° C., Temperature of vaporizer: 225° C., Gas Flow: 12 L/min, Nebulizer: 50.

### Synthesis of tert-butyl (6-(4-formylphenoxy) spiro[3.3]heptan-2-yl)carbamate (INX-SM-43-1)

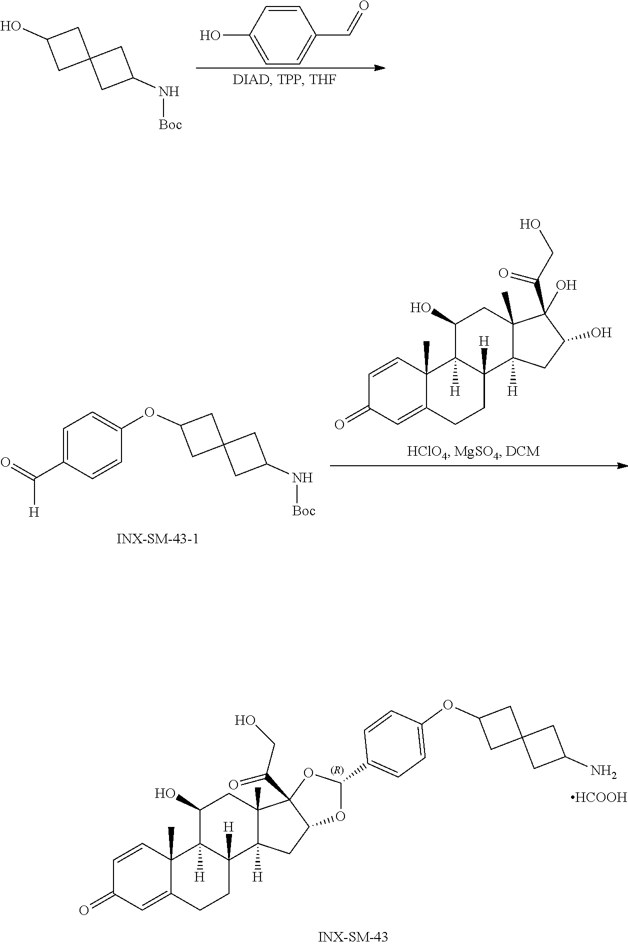

### Synthesis of tert-butyl (6-(4-formylphenoxy) spiro[3.3]heptan-2-yl)carbamate (INX-SM-43-1)

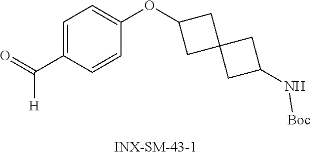 [SMILES: CC(C)(C)OC(=O)NC1CC2(C1)CC(Oc1ccc(C=O)cc1)C2]

A 10 mL glass vial was charged with tert-butyl (6-hydroxyspiro[3.3]heptan-2-yl)carbamate (0.050 g, 0.21 mmol) and 4-hydroxybenzaldehyde (0.053 g, 0.43 mmol) in THF (0.7 mL) under nitrogen. To this solution, triphenylphosphine (0.086 g, 0.32 mmol) and DIAD (0.066 g, 0.32 mmol) were added and stirred at 65° C. for 2 h. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane, 80:20) to give title compound as white solid (0.025 g, 35.9%). LCMS: 332.2 [M+H]⁺, ¹H NMR (400 MHZ, DMOS-d6) δ: 9.84 (s, 1H), 7.82 (d, J=8.8 Hz, 2H), 7.10 (br s, 1H), 7.01 (d, J=8.8 Hz, 2H), 4.72 (quint, 1H), 3.90-3.80 (m, 1H), 2.60-2.40 (m, 4H), 2.25-1.90 (m, 4H), 1.35 (s, 9H).

### Synthesis of (6aR,6bS,7S,8aS,8bS,10R,11aR,12aS,12bS)-10-(4-((6-aminospiro[3.3]heptan-2-yl)oxy) phenyl)-7-hydroxy-8b-(2-hydroxyacetyl)-6a,8a-dimethyl-1,2,6a,6b,7,8,8a,8b,11a,12, 12a,12b-dodecahydro-4H-naphtho[2′,1′:4,5]indeno[1,2-d][1,3]dioxol-4-one (INX-SM-43)

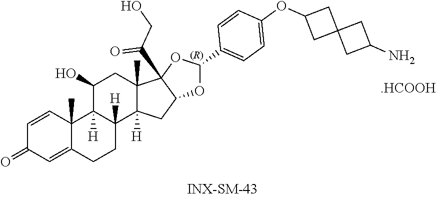

### Procedure

A 10 mL glass vial was charged with tert-butyl (6-(4-formylphenoxy)spiro[3.3]heptan-2-yl)carbamate (INX-SM-43-1) (0.1 g, 0.30 mmol) and (8S,9S,10R,11S,13S,14S,16R,17S)-11,16,17-trihydroxy-17-(2-hydroxyacetyl)-10,13-dimethyl-6,7,8,9,10,11,12,13,14,15, 16, 17-dodecahydro-3H-cyclopenta[a]phenanthren-3-one (16-alpha-hydroxyprednisolone) (0.113 g, 0.30 mmol) in DCM (2 mL). To this solution, MgSO₄ (0.181 g, 1.50 mmol) followed by HClO₄ (0.459 g, 4.53 mmol) were added and stirred for another 2 h at room temperature. After completion of reaction as indicated by TLC, reaction mixture was poured into water and extracted with DCM. The combined organic layer was dried over Na₂SO₄ and evaporated under vacuum. The crude was purified by prep-HPLC (Column: YMC-Actus Triart Prep C18 (250×20) mm, 5 μm, Mobile phase: A=0.1% FA in water, B=Acetonitrile+10% MTBE; A:B=80:20), retention time 17.19 min) to give title compound as white solid (0.017 g, 8.91%). LCMS: 590.4 [M+H]⁺; ¹H NMR (400 MHZ, MeOD): 0 7.46 (d, J=10.0 Hz, 1H), 7.34 (d, J=8.4 Hz, 2H), 6.81 (d, J=8.4 Hz, 2H), 6.26 (d, J=10.0 Hz, 1H), 6.04 (s, 1H), 5.41 (s, Acetal-H, 1H), 5.04 (d, J=4.8 Hz, C16H, 1H), 4.70-4.25 (m, 4H), 3.67-3.63 (m, 1H), 2.70-1.60 (m, 17H), 1.51 (s, 3H), 1.20-1.03 (m, 2H), 0.99 (s, 3H).

A 10 ml glass vial was charged with

### Synthesis of (S)-4-(2-(2-bromoacetamido)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoic acid (INX-A4)

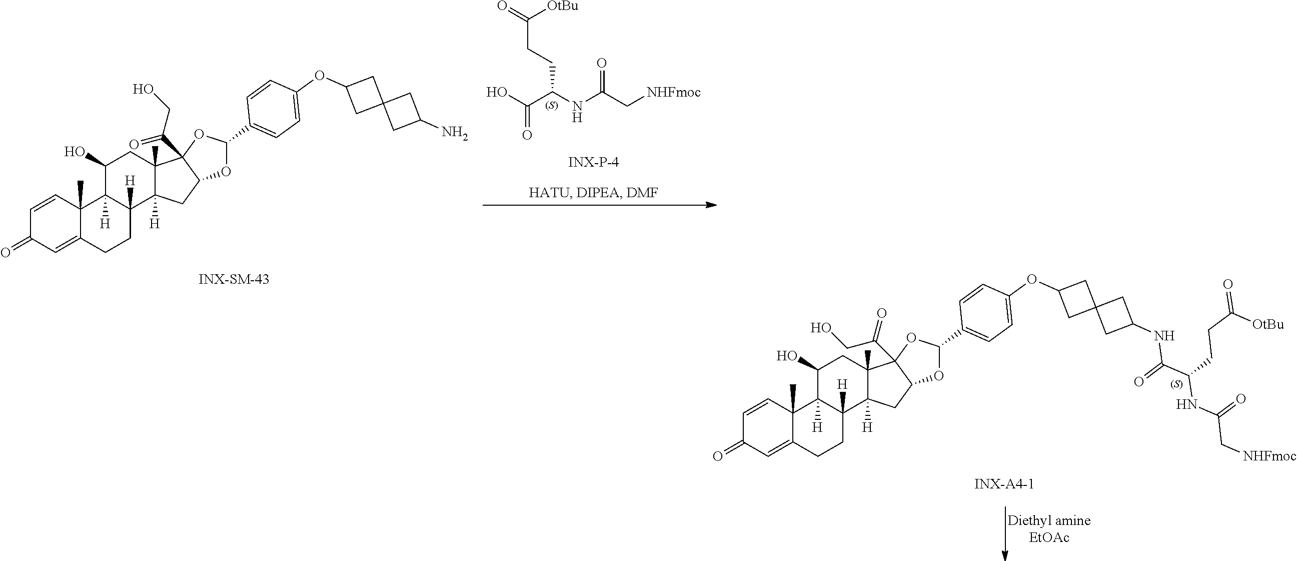

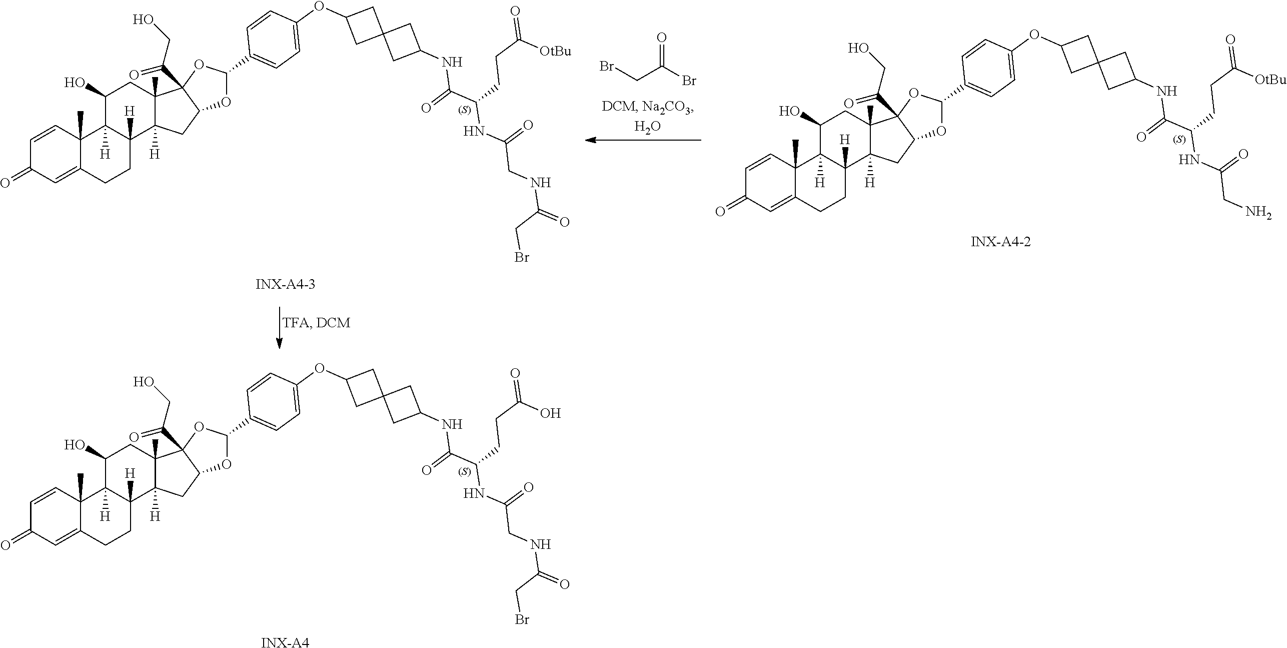

### Synthesis of tert-butyl(S)-4-(2-((((9H-fluoren-9-yl)methoxy)carbonyl)amino)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A4-1)

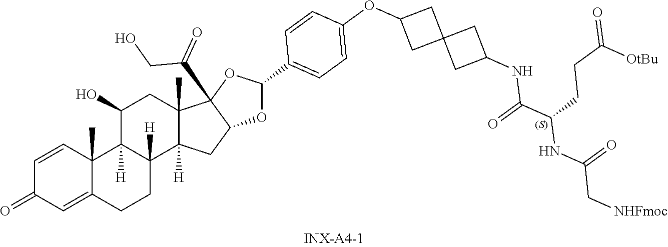

### Procedure

A 50 mL round bottom flask was charged with(S)-2-(2-((((9H-fluoren-9-yl)methoxy)carbonyl)amino)acetamido)-5-(tert-butoxy)-5-oxopentanoic acid (INX-P-4) (0.8 g, 1.65 mmol), DIPEA (0.85 mL, 4.9 mmol) and DMF (8 mL) under nitrogen. To this solution, HATU (1.26 g, 3.31 mmol) and (6aR,6bS,7S,8aS,8bS,10R,11aR,12aS,12bS)-10-(4-((6-aminospiro[3.3]heptan-2-yl)oxy) phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-43) (1.07 g, 1.82 mmol) were added at 0° C. Reaction mixture was allowed to stir at room temperature for 1 h. After completion of reaction as indicated by TLC, reaction mixture was poured into ice cold water and the precipitated solid was filtered and dried under vacuum. The crude solid was triturated with diethyl ether/n-pentane to give title compound as white solid (1.3 g, 74.38%). LCMS: 1054.40 [M+H]⁺.

### Synthesis of tert-butyl(S)-4-(2-aminoacetamido)-5-((6-(4-((6aR,6bS,7S,8aS,8bS,10R,11a R,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A4-2)

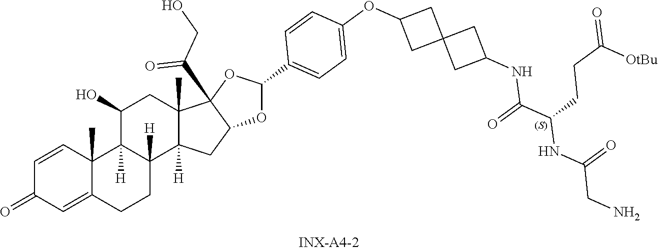

### Procedure

A 100 mL single-necked round bottom flask was charged with tert-butyl(S)-4-(2-((((9H-fluoren-9-yl)methoxy)carbonyl)amino)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A4-1) (1.3 g, 1.23 mmol) and THF (15 mL). To this solution, DEA (2.5 ml, 23.97 mmol) was added at room temperature and stirred for 2 h. After completion of reaction as indicated by TLC, reaction mixture was evaporated under vacuum and triturated with diethyl ether and DCM to give title compound as yellow solid (0.9 g, 88.23%). LCMS: 832.39 [M+H]⁺.

### Synthesis of tert-butyl(S)-4-(2-(2-bromoacetamido)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A4-3)

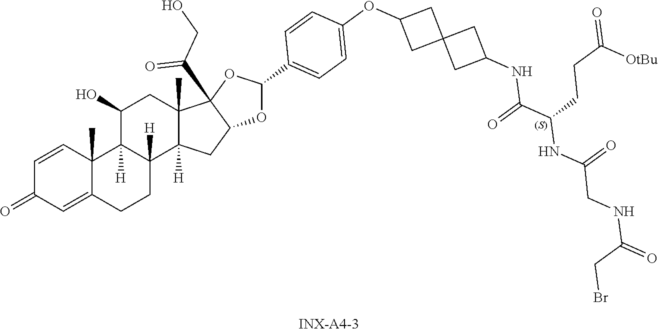

### Procedure

A 100 mL single-necked round bottom flask was charged with tert-butyl(S)-4-(2-aminoacetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A4-2) (0.8 g, 0.961 mmol) and DCM-Water (8:2, 12 mL). To this solution, Na₂CO₃ (0.30 g, 2.88 mmol) followed by bromoacetyl bromine (0.23 g, 1.15 mmol) were added and stirred at room temperature for 4 h. After completion of reaction as indicated by TLC, reaction mixture was poured into water and extracted with DCM. The combined organic layer was dried over Na₂SO₄ and evaporated under vacuum. The crude was purified by silica gel chromatography (MeOH/DCM, 5:95) to give title compound as yellow solid (0.4 g, 43.65%). LCMS: 954.61 [M+H]⁺.

### Synthesis of (S)-4-(2-(2-bromoacetamido)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoic acid (INX-A4)

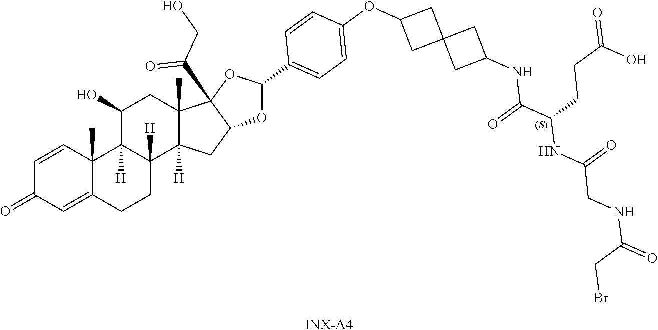

### Procedure

A 25 mL single-necked round bottom flask was charged with tert-butyl(S)-4-(2-(2-bromoacetamido)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A4-3) (0.40 g, 0.420 mmol) and DCM (10 mL). To this solution, TFA (4 mL) was added and stirred for another 4 h at room temperature. After completion of reaction as indicated by TLC, reaction mixture was evaporated under vacuum and triturated with hexane. The crude was purified by prep-HPLC (Column: Xbridge Prep, C18, OBD 19×250 mm, 5 μm, Mobile phase: A=0.05% TFA IN WATER, B=Acetonitrile, A:B=72:28), retention time 16.39 min to give title compound as white solid (0.006 g, 1.59%); LCMS: 896.2 [M+H]⁺; ¹H NMR (400 MHZ, MeOD): 0 7.48 (d, J=10.0 Hz, 1H), 7.36 (d, J=8.4 Hz, 2H), 6.83 (d, J=8.4 Hz, 2H), 6.29 (d, J=10.0 Hz, 1H), 6.06 (s, 1H), 5.43 (s, Acetal-H, 1H), 5.00-3.50 (m, 11H), 2.90-1.60 (m, 21H), 1.53 (s, 3H), 1.20-1.03 (m, 2H), 0.91 (s, 3H).

### Synthesis of (6aR,6bS,7S,8aS,8bS,10R,11aR,12aS,12bS)-10-(4-((6-aminospiro[3.3]heptan-2-yl)oxy)-2-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-44)

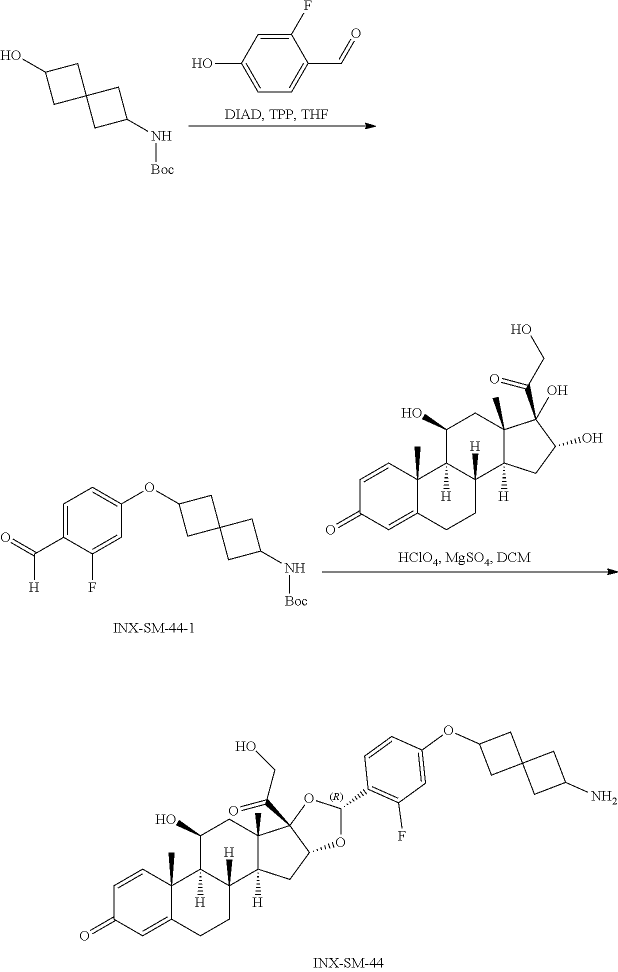

### Synthesis of tert-butyl (6-(3-fluoro-4-formylphenoxy) spiro[3.3]heptan-2-yl)carbamate (INX-SM-44-1)

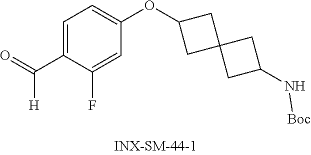 [SMILES: CC(C)(C)OC(=O)NC1CC2(C1)CC(Oc1ccc(C=O)c(F)c1)C2]

### Procedure

A 35 mL glass vial was charged with tert-butyl (6-hydroxyspiro[3.3]heptan-2-yl)carbamate (0.50 g, 2.19 mmol), 2-fluoro-4-hydroxybenzaldehyde (0.61 g, 4.35 mmol), triphenylphosphine (0.86 g, 3.29 mmol) and THF (7 mL) under nitrogen. To this solution, DIAD (0.66 g, 3.29 mmol) was added and heated the reaction mixture for 2 h at 65° C. After completion of reaction as indicated by TLC, reaction mixture was poured into water and extracted with ethyl acetate. The combined organic layer was dried over Na₂SO₄ and evaporated under vacuum. The crude was purified by normal phase column chromatography (ethyl acetate/hexane, 20:80) to give title compound as white solid (0.40 g, 51.97%). LCMS: 350.1[M+H]⁺ (6aR,6bS,7S,8aS,8bS,10R,11aR,12aS,12bS)-10-(4-((6-Synthesis of aminospiro[3.3]heptan-2-yl)oxy)-2-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-44)

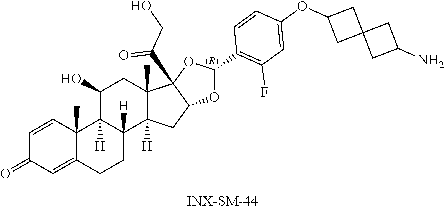

### Procedure

A 25 mL single neck round bottom flask was charged with tert-butyl (6-(3-fluoro-4-formylphenoxy) spiro[3.3]heptan-2-yl)carbamate (INX-SM-44-1) (0.20 g, 0.57 mmol) and (8S,9S,10R,11S,13S,14S,16R,17S)-11,16,17-trihydroxy-17-(2-hydroxyacetyl)-10, 13-dimethyl-6,7,8,9,10,11,12,13,14,15, 16, 17-dodecahydro-3H-cyclopenta[a]phenanthren-3-one (0.215 g, 0.57 mmol) in DCM (5 mL). To this solution, MgSO₄ (0.34 g, 2.86 mmol) and HClO₄ (0.57 g, 5.72 mmol) were added and stirred for 2 h at room temperature. After completion of reaction as indicated by TLC, reaction mixture was quenched with sat. NaHCO₃ solution and concentrated under vacuum. Then crude was triturated with cold water and precipitated solid was filtered and dried under vacuum. The crude was purified by reverse phase column chromatography (water: acetonitrile, 60:40) to give title compound as white solid (0.020 g, 5.75%). LCMS: 608.4 [M+H]⁺; ¹H NMR (400 MHZ, MeOD, Key proton assignment): δ: 7.46-7.42 (m, 2H), 6.67-6.55 (m, 2H), 6.25 (d, J=10 Hz, 1H), 6.27 (s, 1H), 5.64 (s, Acetal-H, 1H), 5.04-5.03 (d, J=4.8 Hz, C16H, 1H), 4.63-4.29 (m, 4H), 3.64-3.60 (m, 1H), 2.70-1.73 (m, 17H), 1.50 (s, 3H), 1.30-1.12 (m, 2H), 0.92 (s, 3H).

### Synthesis of (S)-4-(2-(2-bromoacetamido)acetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoic acid (INX-A5)

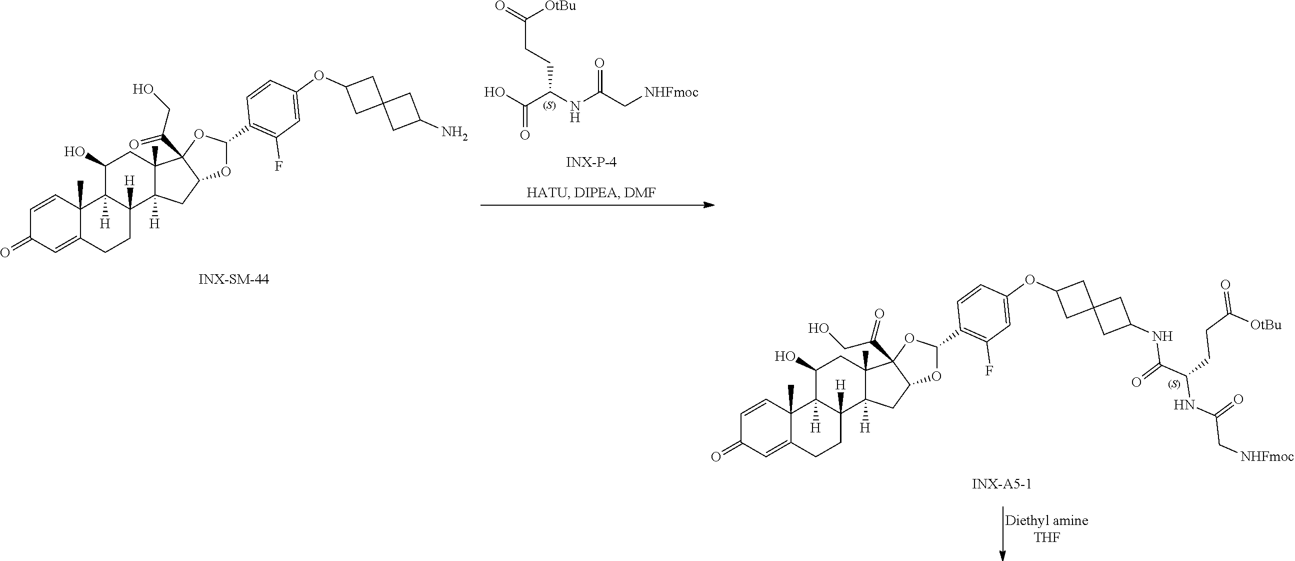

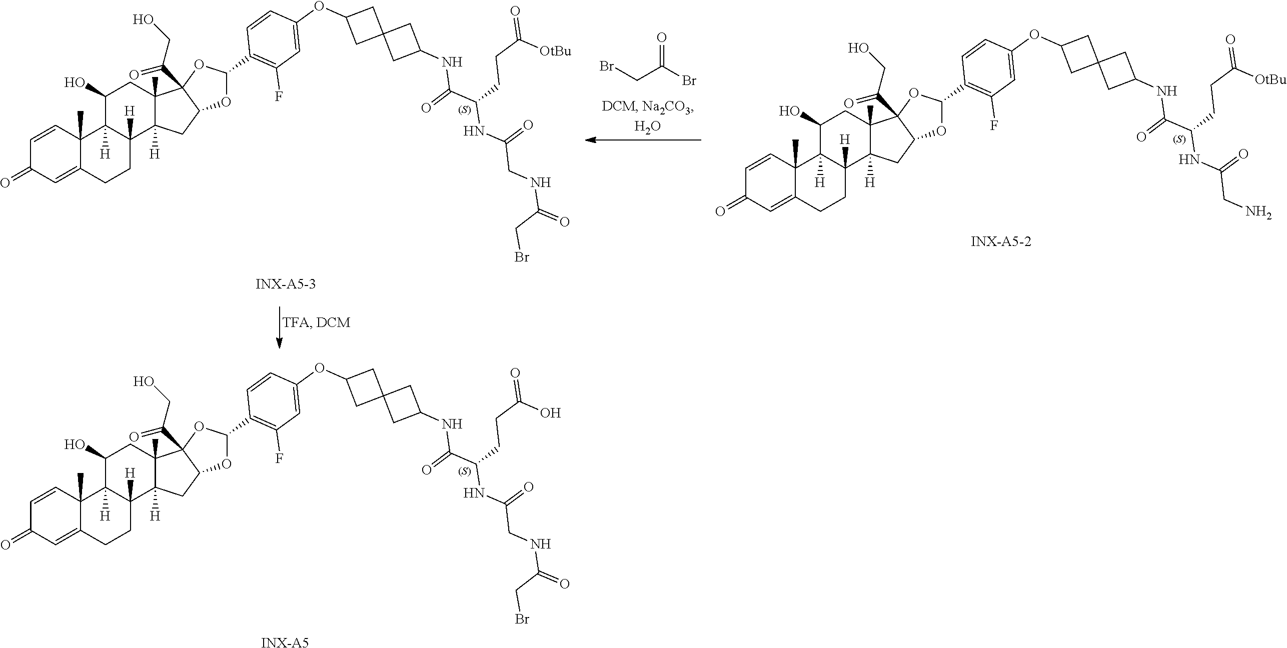

### Synthesis of tert-butyl(S)-4-(2-((((9H-fluoren-9-yl)methoxy)carbonyl)amino)acetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A5-1)

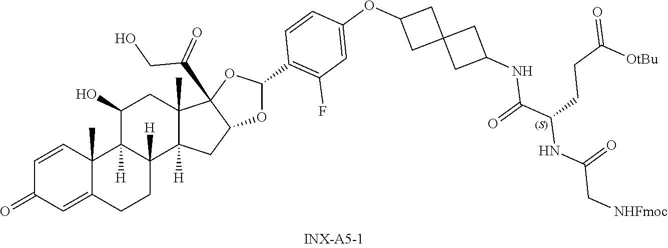

### Procedure

A 50 mL round bottom flask was charged with(S)-2-(2-((((9H-fluoren-9-yl)methoxy)carbonyl)amino)acetamido)-5-(tert-butoxy)-5-oxopentanoic acid (INX-P-4) (1.0 g, 2.07 mmol) and DMF (10 mL) under nitrogen and cooled at 0° C. To this solution, HATU (1.57 g, 4.14 mmol), (6aR,6bS,7S,8aS,8bS,10R,11aR,12aS,12bS)-10-(4-((6-aminospiro[3.3]heptan-2-yl)oxy)-2-fluorophenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-44) (1.25 g, 2.07 mmol) then DIPEA (1.0 mL, 6.22 mmol) were added at 0° C. Reaction mixture was allowed to stir at room temperature for 1 h. After completion of reaction as indicated by TLC, reaction mixture was poured into ice cold water. The resulting solid was filtered and dried under vacuum. The solid was triturated with diethyl ether and n-pentane to give title compound as white solid (1.3 g, 59.09%). LCMS: 1072.8 [M+H]⁺.

### Synthesis of tert-butyl(S)-4-(2-aminoacetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A5-2)

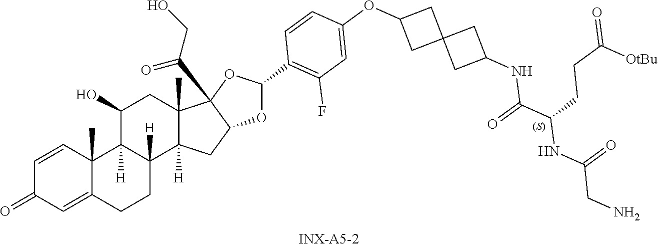

### Procedure

A 50 mL single-necked round bottom flask was charged with tert-butyl(S)-4-(2-((((9H-fluoren-9-yl)methoxy)carbonyl)amino)acetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A5-1) (1.3 g, 1.21 mmol) and THF (10 mL). To this solution, DEA (1.8 ml, 17.26 mmol) was added and stirred for 2 h at room temperature. After completion of reaction as indicated by TLC, reaction mixture was evaporated under vacuum and triturated with diethyl ether and DCM to give title compound as yellow solid (0.9 g, 87.37%). LCMS: 850.5 [M+H]⁺.

### Synthesis of tert-butyl(S)-4-(2-(2-bromoacetamido)acetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A5-3)

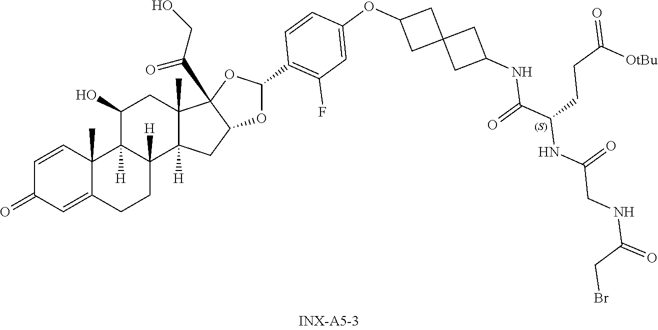

A 25 mL single-necked round bottom flask was charged with tert-butyl(S)-4-(2-aminoacetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A5-2) (0.9 g, 1.05 mmol) and DCM-Water (8:2, 12 mL). To this solution, Na₂CO₃ (0.33 g, 3.17 mmol) followed by bromoacetyl bromine (0.25 g, 1.27 mmol) were added and stirred for 4 h at room temperature. After completion of reaction as indicated by TLC, reaction mixture was poured into water and extracted with DCM. The combined organic layer was dried over Na₂SO₄ and evaporated under vacuum. The crude was purified by silica gel chromatography (MeOH/DCM, 5:95) to give title compound as yellow solid (0.5 g, 49.01%). LCMS: calculated for C₄₈H₆₂⁷⁹BrFN₃O₁₂ (970.35), found 970.40 [M+H]⁺.

### Synthesis of (S)-4-(2-(2-bromoacetamido)acetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoic acid (INX-A5)

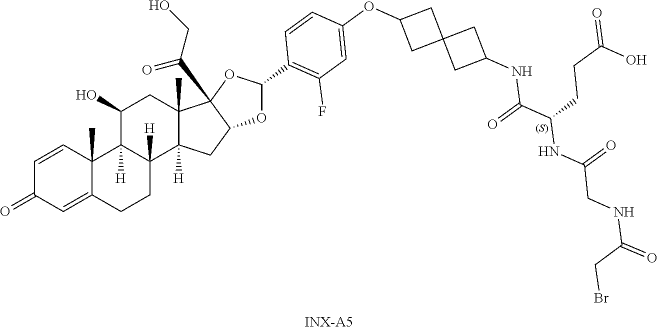

### Procedure

A 25 mL single-necked round bottom flask was charged with tert-butyl(S)-4-(2-(2-bromoacetamido)acetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A5-3) (0.2 g, 0.0.205 mmol) and DCM (7 mL). To this solution, TFA (3 mL) was added and stirred at room temperature for 4 h. After completion of reaction as indicated by TLC, reaction mixture was evaporated under vacuum and triturated with hexane. The crude was purified by prep-HPLC (Column: SUNFIRE Prep C18 OBD, 19×250 mm, 5 μm 12 nm, Mobile phase: A=0.05% TFA in water, B=Acetonitrile, A:B=45:55, retention time 12 min) to give title compound as white solid (0.006 g, 3.18%);

LCMS: calculated for C₄₄H₅₄⁷⁹BrFN₃O₁₂ (914.29), found 914.5 [M+H]⁺; ¹H NMR (400 MHZ, MeOD): 0 7.47-7.42 (m, 2H), 6.68 (dd, J=8.4 & 2 Hz, 1H), 6.59 (dd, J=12.4 & 2.4 Hz, 1H), 6.27 (J=10.0 & 2.0 Hz, 1H), 6.04 (s, 1H), 5.65 (s, 1H, Acetal-H), 5.03 (d, J=5.2 Hz, 1H, C16H), 4.64-4.55 (m, 2H), 4.44-4.43 (m, 1H), 4.34-4.30 (m, 2H), 4.22-4.16 (m, 1H), 3.95 (d, 2H), 3.92-3.90 (m, 2H), 2.80-1.80 (m, 21H), 1.51 (s, 3H), 1.25-1.15 (m, 2H), 1.00 (s, 3H).

### Synthesis of (INX-J2)

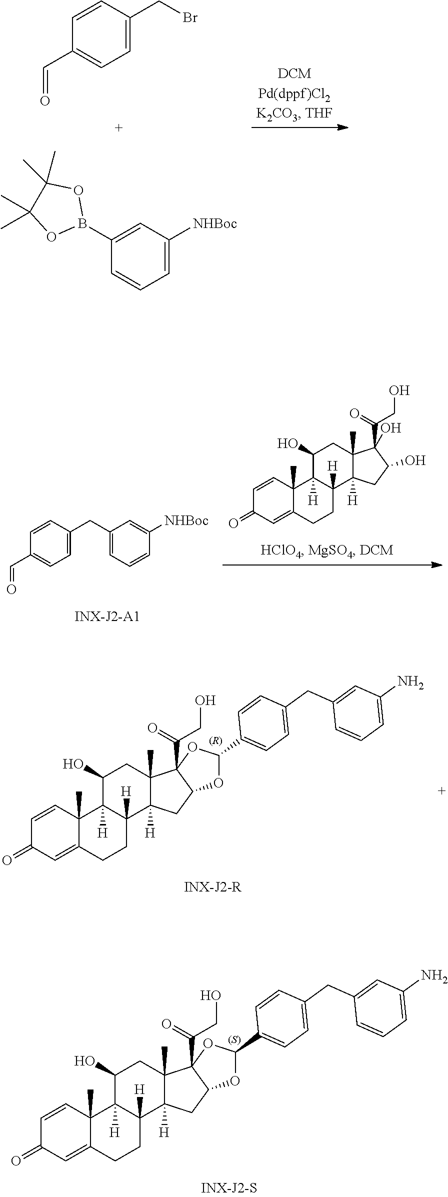

### Synthesis of tert-butyl (3-(4-formylbenzyl)phenyl)carbamate (INX-J2-A1)

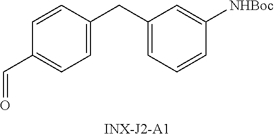 [SMILES: CC(C)(C)OC(=O)Nc1cccc(Cc2ccc(C=O)cc2)c1]

### Procedure

A 50 ml single necked round bottom flask was charged with 4-(bromomethyl) benzaldehyde (0.5 g, 2.5 mmol) and THF (5 ml). To this solution, tert-butyl (3-(4,4,5,5-tetramethyl-1,3,2-dioxaborolan-2-yl)phenyl)carbamate (1.2 g, 3.7 mmol) and potassium carbonate (0.8 g, 6.2 mmol) were added and purged with N₂ for 15 min. PdCl₂·dppf-DCM (0.3 g, 0.15 mmol) was added to the reaction mixture and allowed to stir for 16 h at 80° C. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate:hexane, 5:95) to give title compound as light yellow solid (0.32 g, 40.95%). LCMS: 312.4 [M+H]⁺.

### Synthesis of (6aR,6bS,7S,8aS,8bS,10R,11aR,12aS,12bS)-10-(4-(3-aminobenzyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-J2-R); and (6aR,6bS,7S,8aS,8bS,10S,11aR,12aS,12bS)-10-(4-(3-aminobenzyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-J2-S)

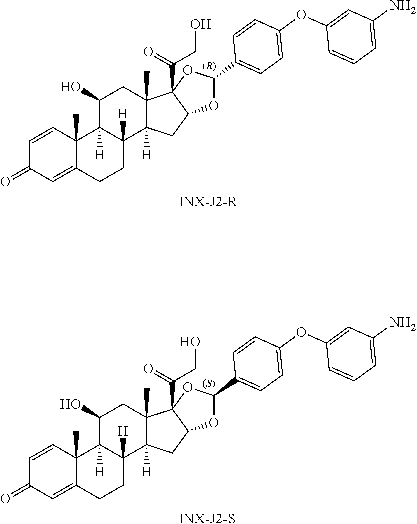

A 10 mL single-necked round bottom flash was charged with (tert-butyl (3-(4-formylbenzyl)phenyl)carbamate (INX-J2-A1) (0.30 g, 0.96 mmol), (8S,9S,10R,11S,13S,14S,16R,17S)-11,16,17-trihydroxy-17-(2-hydroxyacetyl)-10,13-dimethyl-6,7,8,9,10,11,12,13,14,15,16, 17-dodecahydro-3H-cyclopenta[a]phenanthren-3-one (16 alpha-hydroxyprednisole) (0.36 g, 0.96 mmol) was dissolved and DCM (10 mL). To this solution, MgSO₄ (0.57 g, 4.8 mmol) was added and stirred for a 5 min at room temperature. HClO₄ (0.48 g, 4.82 mmol) was added to the reaction mixture and stirred at room temperature for another 3 h. After completion of reaction as indicated by TLC, reaction mixture was quenched with NaHCO₃ solution and extracted with DCM. The combined organic layer was dried over Na₂SO₄ and evaporated under vacuum. The crude was purified by prep HPLC (Column: VIRDIS prep silica, 2EP-OBD, 250×19 mm S-5 μm, Mobile phase: A=0.1% ammonia in heptane, B=IPA: MTBE (70:30); A:B, 80:20) to give Isomer-1 and Isomer-2. These isomers were eluted at retention time 25.49 min (INX-J2-S), and 31.53 min (INX-J2-R). INX-J2-R (Isomer-2, R-Isomer): (Yield: 0.10 g, 18.2%). LCMS: 570.4 [M+H]⁺; ¹H NMR (400 MHz, MeOD): δ: 7.45 (d, J=10 Hz, 1H), 7.35 (d, J=8.0 Hz, 2H), 7.20 (d, J=7.6 Hz, 2H), 7.00 (t, 1H), 6.56-6.52 (m, 3H), 6.27 (d, J=10 Hz, H), 6.03 (s, 1H), 5.44 (s, 1H, Acetal-H), 5.06 (d, J=5.2 Hz, 1H, C16H), 4.63 (d, J=19.2 Hz, 1H), 4.43 (brs, 1H), 4.33 (d, J=19.2 Hz, 1H), 3.85 (s, 2H), 2.70-1.600 (m, 9H), 1.51 (s, 3H), 1.20-1.00 (m, 2H), 0.93 (s, 3H).

INX-J2-S(Isomer-1, S-Isomer): (Yield: 0.010 g, 1.82%). LCMS 570.4 [M+H]⁺; ¹H NMR (400 MHz, MeOD): δ: 7.49 (d, J=10 Hz, 1H), 7.23-7.17 (m, 4H), 7.01 (t, 1H), 6.57-6.52 (m, 3H), 6.27 (d, J=10 Hz, 1H), 6.12 (s, Acetal-H, 1H), 6.04 (s, 1H), 5.40 (d, J=6.4 Hz, C16H, 1H), 4.43 (br s, 1H), 4.33 (d, J=19.2 Hz, 1H), 4.12 (d, J=19.2 Hz, 1H), 3.85 (s, 2H), 2.70-1.60 (m, 9H), 1.52 (s, 3H), 1.20-1.00 (m, 2H), 0.93 (s, 3H).

### Synthesis of S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid (INX-J)

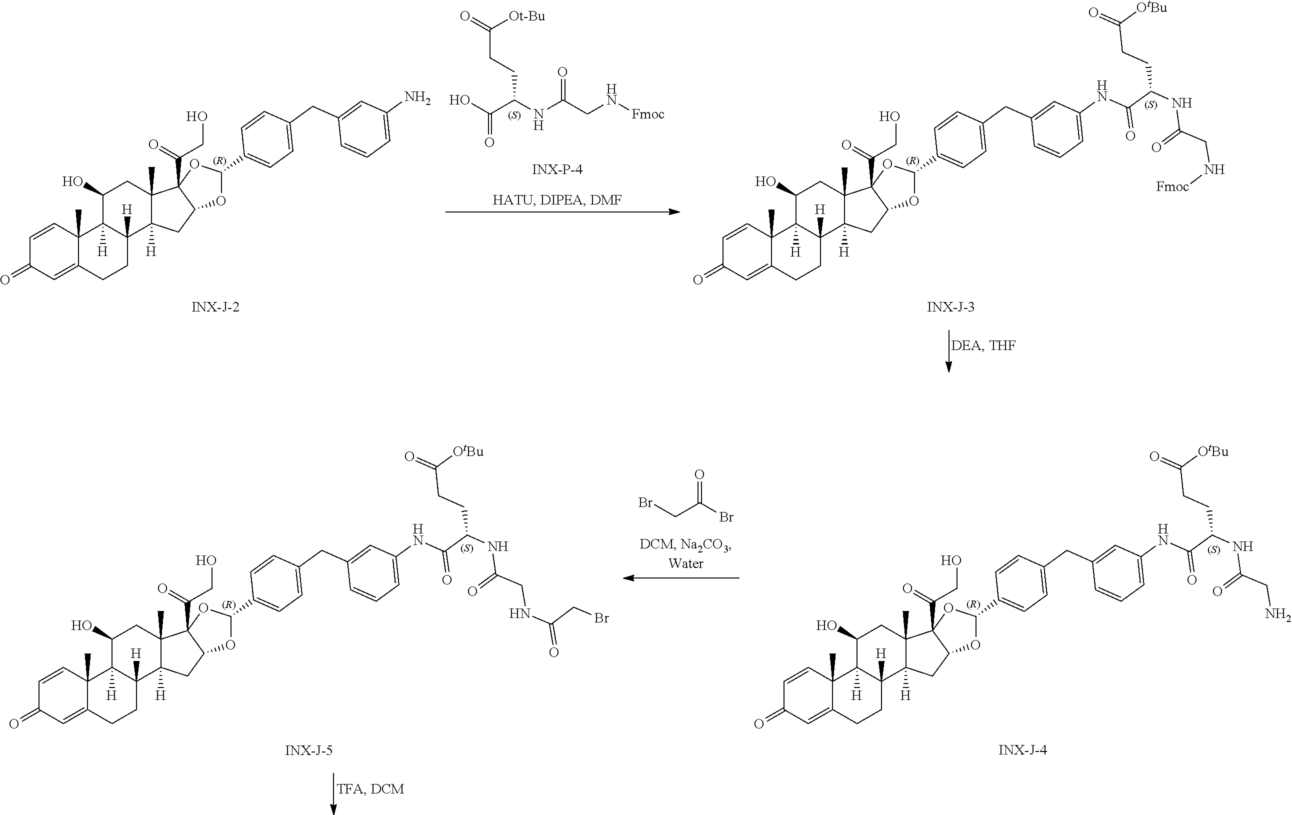

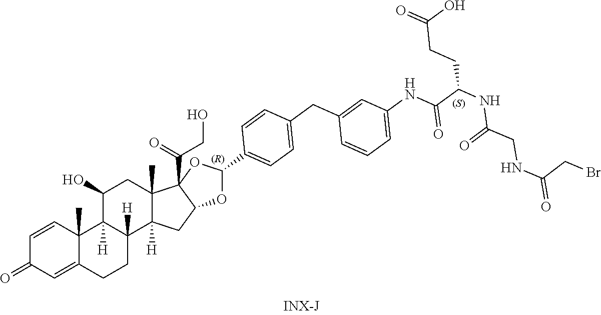

### Synthesis of tert-butyl(S)-4-(2-((((9H-fluoren-9-yl)methoxy)carbonyl)amino)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (INX-J-3)

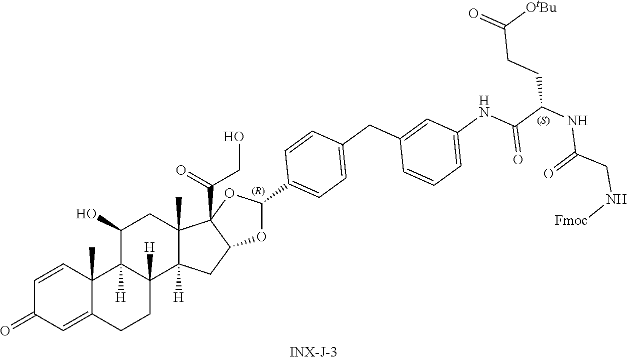

### Procedure

A 50 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) (0.8 g, 1.65 mmol), HATU (0.94, 2.47 mmol), DIPEA (0.42 g, 3.30 mmol) and DMF (8 mL) at room temperature under N₂. To this solution, (6aR,6bS,7S,8aS,8bS,10R,11aR,12aS,12bS)-10-(4-(3-aminobenzyl)phenyl)-7-hydroxy-8b-(2-hydroxyacetyl)-6a,8a-dimethyl 1,2,6a,6b,7,8,8a,8b,11a,12,12a,12b-dodecahydro-4H-naphtho[2′,1′:4,5]indeno[1,2-d][1,3]dioxol-4-one (INX-J-2-R) (0.93 g, 1.65 mmol) was added at room temperature and stirred for 1 h. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water: 60:40) to give title compound as light yellow solid (0.4 g, 23.31%). LCMS: 1034.8 [M+H]⁺.

### Synthesis of tert-butyl(S)-4-(2-aminoacetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (INX-J-4)

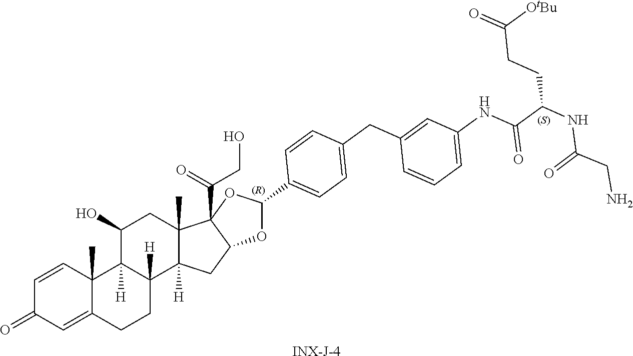

### Procedure

A 50 mL single-necked round bottom flash was charged with tert-butyl(S)-4-(2-((((9H-fluoren-9-yl)methoxy)carbonyl)amino)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (INX-J3) (0.4 g, 0.38 mmol) and THF (4 mL). To this solution, diethylamine (0.27 g, 3.8 mmol) was added at room temperature and stirred for 3 h. After completion of reaction as indicated by TLC, reaction mixture was evaporated under vacuum to give title compound as yellow solid (0.3 g, crude) LCMS: 812.5 [M+H]⁺.

### Synthesis of tert-butyl(S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (INX-J-5)

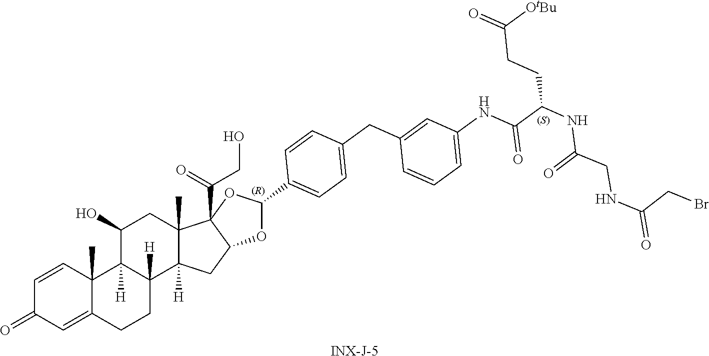

### Procedure

A 25 mL single-necked round bottom flash was charged with tert-butyl(S)-4-(2-aminoacetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (INX-J-4) (0.3 g, 0.36 mmol) and DCM (3 mL). To this solution, Na₂CO₃ (0.075 g, 0.72 mmol) dissolved in water (0.5 mL) followed by bromoacetyl bromide (0.088 g, 0.43 mmol) were added at room temperature and stirred for further 1 h. After completion of reaction as indicated by TLC, reaction mixture was quenched with water and extracted with DCM. The combined organic layer was dried over Na₂SO₄ and evaporated under vacuum. The crude was purified by trituration with diethyl ether to give title compound as pale yellow solid (0.27 g, 78.29%).

LCMS: 932.5[M+H]⁺

### Synthesis of (S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid (INX-J)

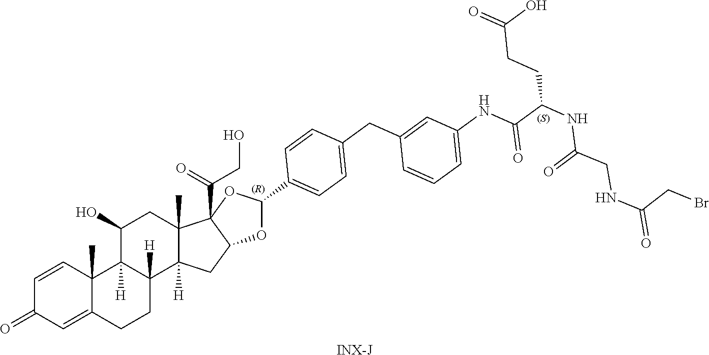

### Procedure

A 10 mL single-necked round bottom flash was charged with tert-butyl(S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoate (INX-J5) (0.27 g, 0.28 mmol) in DCM (2 mL). To this solution, TFA (0.15 g, 1.40 mmol) was added and stirred at room temperature for 2 h. After completion of reaction as indicated by TLC, reaction mixture was evaporated under vacuum. The crude was purified by prep-HPLC (Column: C18 (250*21.2) mm, 5 μm, Mobile phase: A=0.05% TFA in water, B=acetonitrile, A:B=48:52), retention time 12.5 min) to give title compound as off-white solid (0.020 g, 10.62%). LCMS: calculated for C₄₄H₅₁⁷⁹BrN₃O₁₁ (876.27), found 876.40 [M+H]⁺; ¹H NMR (400 MHZ, MeOD): 0 7.47-7.36 (m, 5H), 7.23-7.20 (m, 3H), 6.96 (d, 1H), 6.27 (d, J=10 Hz, 1H), 6.04 (s, 1H), 5.45 (s, Acetal-H, 1H), 5.06 (d, J=5.2 Hz, C16H, 1H), 4.70-4.20 (m, 4H), 4.10-4.00 (m, 6H), 2.70-1.60 (m, 13H), 1.50 (s, 3H), 1.10-1.02 (m, 2H), 1.00 (3H).

### Synthesis of (S)-4-(2-(2-(((R)-2-amino-2-carboxyethyl) thio) acetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid (INX-J-CYS)

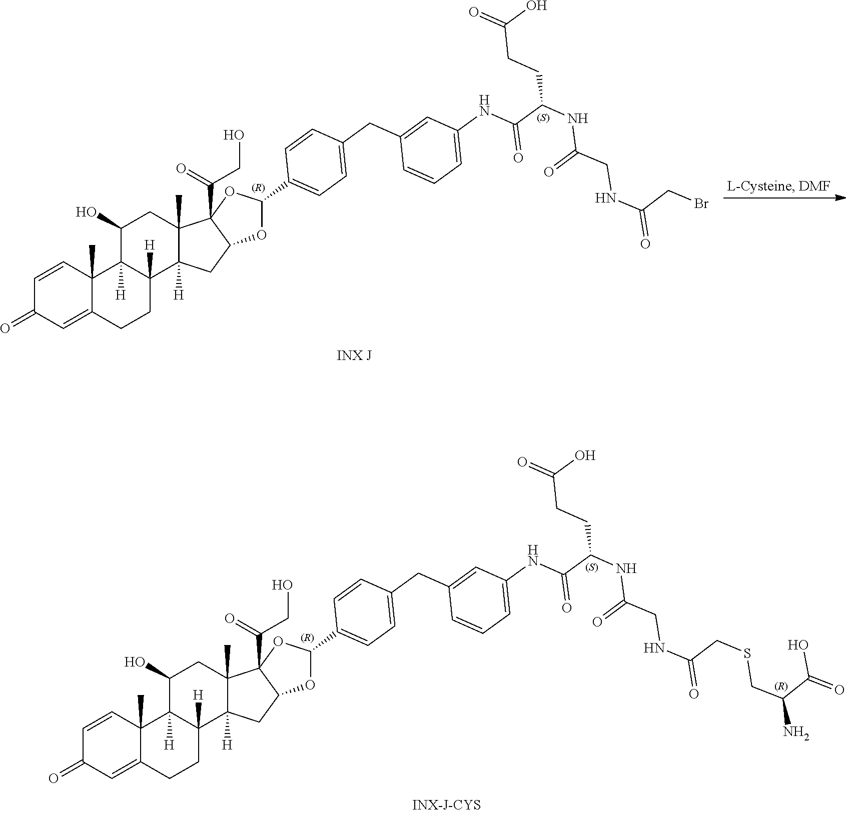

### Procedure

A 10 mL single-necked round bottom flash was charged with(S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)phenyl)amino)-5-oxopentanoic acid (INX-J) (0.2 g, 0.22 mmol) and DMF (1 mL). To this solution, L-cysteine (0.041 g, 0.34 mmol) was added and stirred for 2 h at room temperature. After completion of reaction as indicated by LCMS, reaction mixture was lyophilized and the crude was purified by prep HPLC (Column: YMC-Actus Triart Prep C18-S, 250×20 mm S-5 μm, 12 nm, Mobile phase: A=0.05% TFA in water, B=acetonitrile, A:B=78:22), retention time 20 min) to give title compound as off white solid (0.010 g, 4.78%). LCMS: calculated for C₄₇H₅₇⁷⁹BrN₄O₁₃S (917.36), found 917.4 [M+H]⁺.

¹H NMR (400 MHZ, MeOD): 0 7.47-7.36 (m, 5H), 7.24-7.20 (m, 3H), 6.96 (d, 1H), 6.27 (d, J=10 Hz, 1H), 6.04 (s, 1H), 5.45 (s, Acetal-H, 1H), 5.06 (d, J=5.2 Hz, C16H, 1H), 4.70-4.20 (m, 4H), 4.10-3.90 (m, 5H), 3.50-3.00 (m, 4H), 2.70-1.60 (m, 13H), 1.50 (s, 3H), 1.10-1.02 (m, 2H), 1.00 (3H).

### Synthesis of (2S,6aS,6bR,7S,8aS,8bS,10R,11aR,12aS,12bS)-10-(4-((6-aminospiro[3.3]heptan-2-yl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-25)

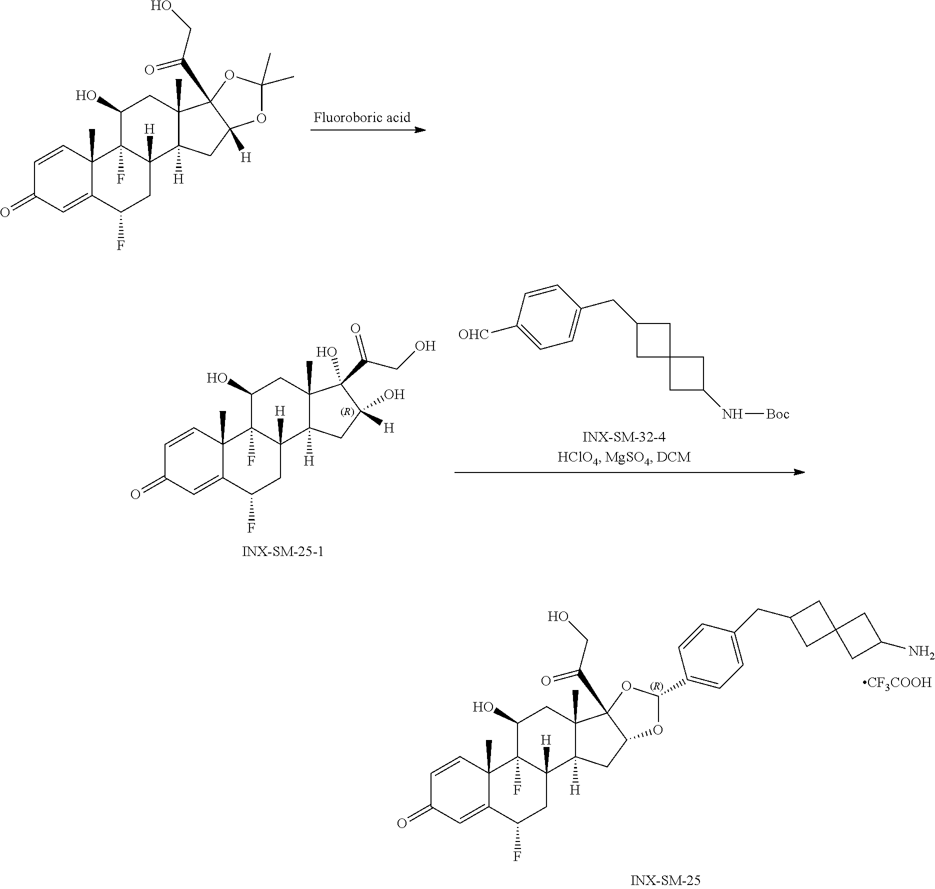

### Synthesis of (6S,8S,9R,10S,11S,13S,14S,16R,17S)-6,9-difluoro-11,16, 17-trihydroxy-17-(2-hydroxyacetyl)-10,13-dimethyl-6,7,8,9,10,11,12,13,14,15,16,17-dodecahydro-3H-cyclopenta[a]phenanthren-3-one (INX-SM-25-1)

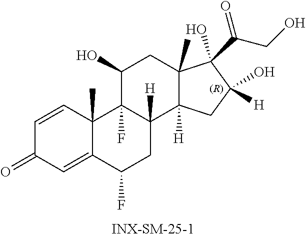

### Procedure

A 35 mL glass vial was charged with Fluocinolone acetonide (1 g, 2.23 mmol) and fluoroboric acid (10 mL). The mixture was stirred at room temperature for 16 h. After completion of reaction as indicated by TLC, reaction mixture was poured into water and the resulted solid was filtered and dried under vacuum to give title compound as white solid (0.8 g, 87.77%).

LCMS: 413.2 [M+H]⁺.

### Synthesis of (2S,6aS,6bR,7S,8aS,8bS,10R,11aR,12aS,12bS)-10-(4-((6-aminospiro[3.3]heptan-2-yl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-25)

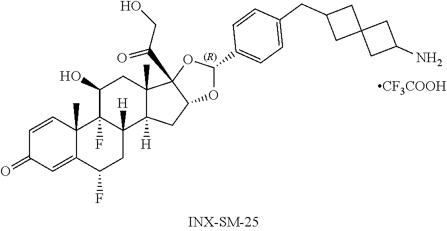

### Procedure

A 25 mL single-necked round bottom flash was charged with tert-butyl (6-(4-formylbenzyl) spiro[3.3]heptan-2-yl)carbamate (INX-SM-32-4) (0.10 g, 0.33 mmol), (6S,8S,9R,10S,11S,13S,14S,16R,17S)-6,9-difluoro-11,16,17-trihydroxy-17-(2-hydroxyacetyl)-10, 13-dimethyl-6,7,8,9, 10, 11, 12, 13, 14, 15, 16, 17-dodecahydro-3H-cyclopenta[a]phenanthren-3-one (INX-SM-25-1) (0.1 g, 0.24 mmol) and DCM (3 mL). To this solution, MgSO₄ (0.18 g, 1.59 mmol) and HClO₄ (0.15 g, 1.59 mmol) were added and stirred for 2 h at room temperature. After completion of reaction as indicated by TLC, reaction mixture was poured into water and extracted with ethyl acetate. The combined organic layer was dried over Na₂SO₄ and evaporated under vacuum. The crude was purified by prep-HPLC (Column: SUNFIRE Prep C18 OBD, 19×250 mm, 5 μm, Mobile phase: A=0.05% TFA in water, B=Acetonitrile, A:B=70:30), retention time 9.98 min) to give title compound as white solid (0.024 g, 13.40%). LCMS: 624.4 [M+H]⁺; ¹H NMR (400 MHZ, MeOD,): δ: 8.55 (br s, 1H), 7.36-7.33 (m, 3H), 7.16 (d, J=8.0 Hz, 2H), 6.36-6.33 (m, 2H), 5.66-5.51 (m, J=45.6 Hz, CHF, 1H) 5.48 (s, Acetal-H, 1H), 5.07 (d, J=3.2 Hz, C16H, 1H), 5.07-5.06 (m, 1H), 4.63 (d, J=19.2 Hz, 1H), 4.36-4.31 (m, 2H), 3.58-3.54 (m, 1H), 2.75-1.60 (m, 18H), 1.60 (s, 3H), 1.00 (s, 3H).

### Synthesis of (S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX-A23)

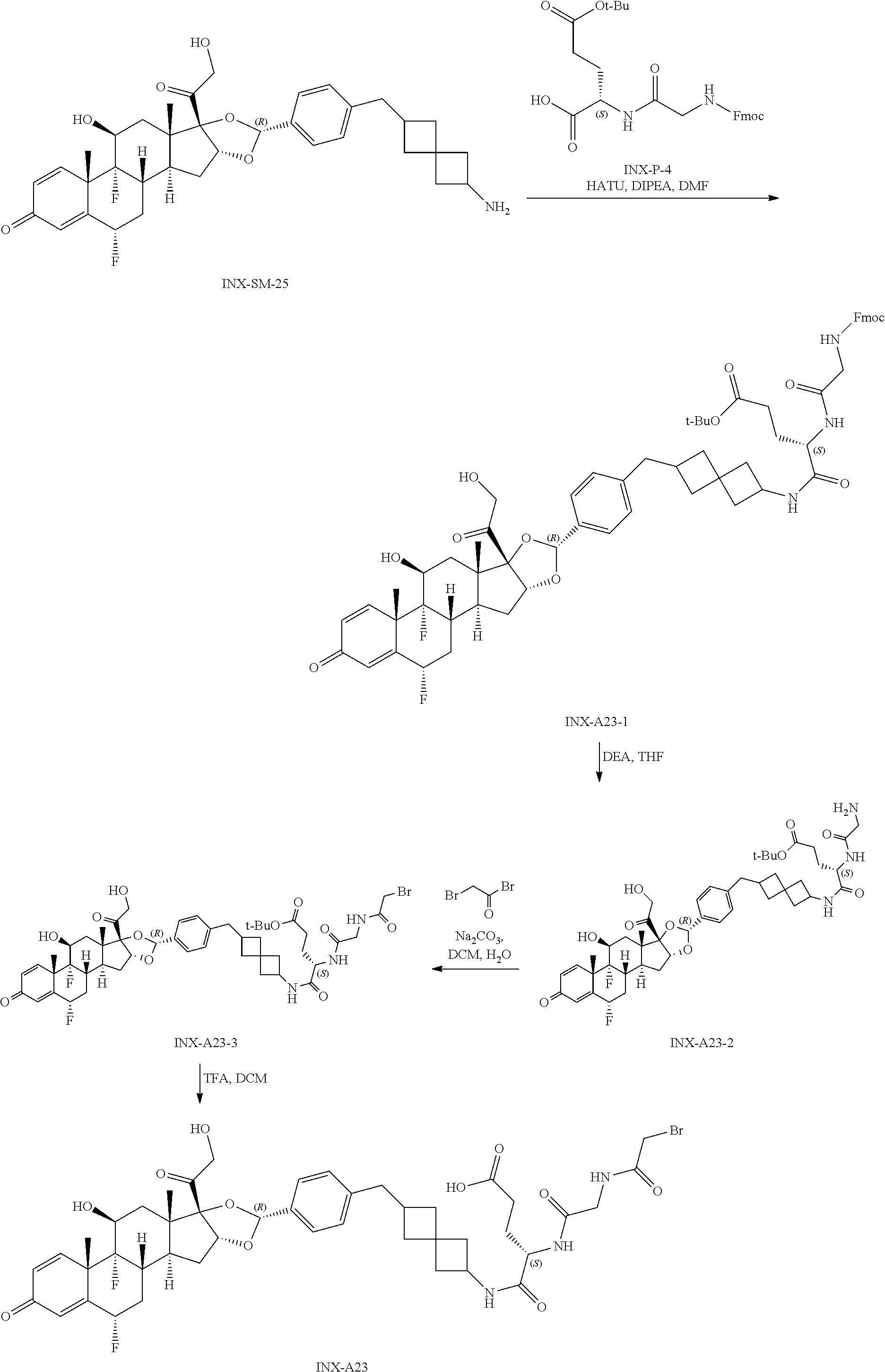

### Synthesis of tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A23-1)

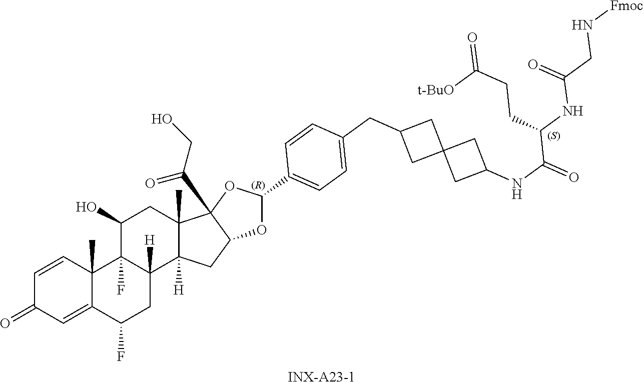

### Procedure

A 10 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) (0.38 g, 0.78 mmol), HATU (0.608 g, 1.6 mmol) and DMF (5 mL). To this solution, DIPEA (0.25 g, 2.0 mmol) followed by (2S,6aS,6bR,7S,8aS,8bS,10R,11aR,12aS,12bS)-10-(4-((6-aminospiro[3.3]heptan-2-yl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-25) (0.5 g, 0.80 mmol) was added at room temperature and stirred for 1 h. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water: 50:50) to give title compound as light yellow solid (0.3 g, 34.39%). LCMS 1088.6 [M+H]⁺.

### Synthesis of tert-butyl(S)-4-(2-aminoacetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A23-2)

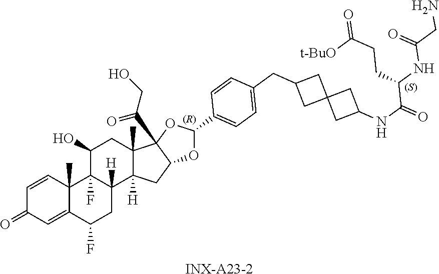

### Procedure

A 10 mL single-necked round bottom flash was charged with tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A23-1) (0.12 g, 0.11 mmol) and THF (2 mL). To this solution, diethyl amine (0.08 g, 1.10 mmol) was added at room temperature and stirred for 3 h. After completion of reaction as indicated by TLC, reaction mixture was evaporated under vacuum and triturated with diethyl ether to give title compound as yellow solid (0.08 g, 83.78%) LCMS: 866.6 [M+H]⁺.

### Synthesis of tert-butyl(S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A23-3)

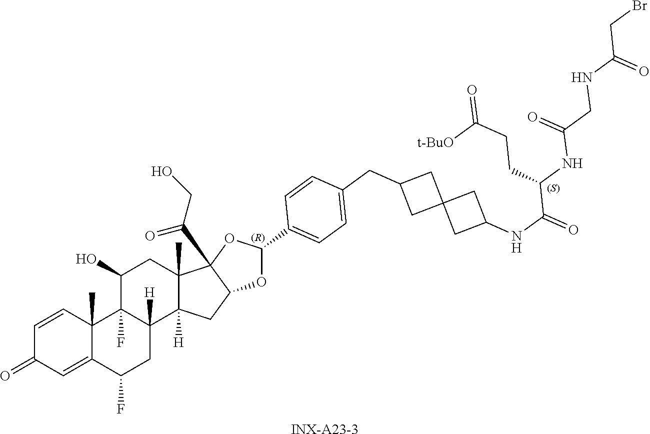

### Procedure

A 10 mL single-necked round bottom flash was charged with tert-butyl(S)-4-(2-aminoacetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A23-2) (0.08 g, 0.09 mmol) and DCM (3 mL). To this solution, Na₂CO₃ (0.039 g, 0.36 mmol) dissolved in water (1 mL) followed by bromoacetyl bromide (0.037 g, 0.18 mmol) were added and stirred for 1 h at room temperature. After completion of reaction as indicated by TLC, reaction mixture was quenched with water and extracted with DCM. The combined organic layer was dried over Na₂SO₄ and evaporated under vacuum. The crude was purified by reverse phase column chromatography (acetonitrile:water, 50:50) to give title compound as light yellow (0.080 g, 40%). LCMS: 986.6 [M+H]⁺.

### Synthesis of (S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX-A23)

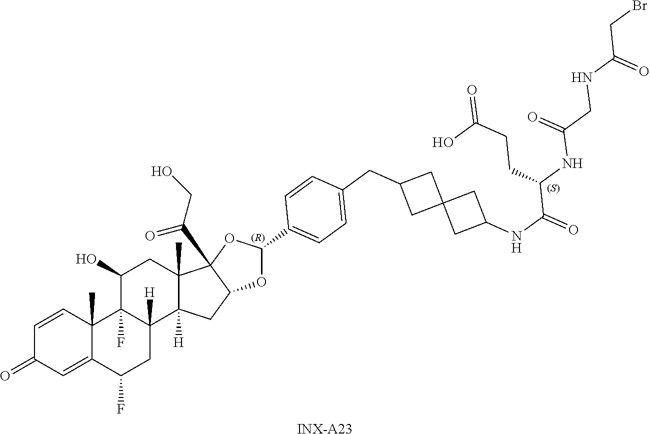

### Procedure

A 10 mL single-necked round bottom flash was charged with tert-butyl(S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A23-3) (0.080 g, 0.08 mmol) and DCM (2 mL). To this solution, TFA (0.048 g, 0.45 mmol) was added and stirred for 2 h at room temperature. After completion of reaction as indicated by TLC, reaction mixture was evaporated under vacuum. The crude was purified by prep-HPLC (Column: SUNFIRE Prep C18 OBD, 19×250 mm, 5 μm, Mobile phase: A=0.05% TFA in water, B=acetonitrile; A:B=58:42), retention time 14.62 min) to give title compound as off white solid (0.008 g, 10.60%). LCMS: calculated for C₄₅H₅₄⁷⁹BrF₂N₃O₁₁Na (953.80), found 953.5 [M+Na]⁺; ¹H NMR (400 MHZ, MeOD, Key proton assignment): δ: 7.36-7.34 (m, 3H), 7.17 (d, J=8.0 Hz, 2H), 6.38-6.35 (m, 2H), 5.66-5.49 (m, CHF, 1H), 5.48 (s, 1H, Acetal-H), 5.07 (d, J=4.4 Hz, 1H, C16H), 4.64 (d, 1H), 4.37-4.32 (m, 2H), 4.15-4.10 (m, 1H), 3.94-3.90 (m, 4H), 2.68 (d, 2H), 2.50-1.65 (m, 22H), 1.60 (s, 3H), 0.90 (s, 3H).

### Synthesis of (S)-6-amino-2-(2-(2-bromoacetamido)acetamido)-N-(6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl) hexanamide (INX-A6)

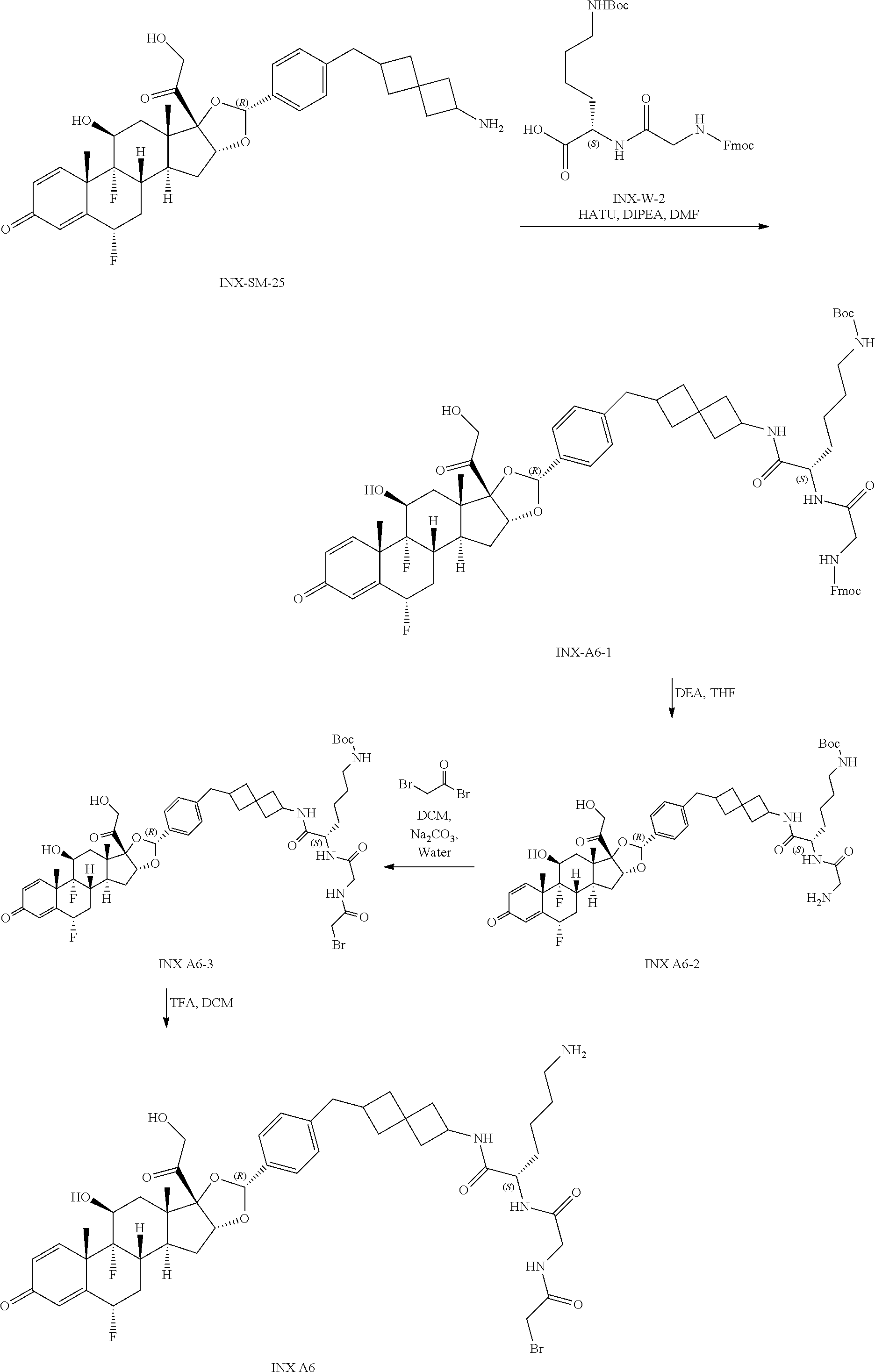

### Synthesis of tert-butyl ((S)-5-(2-((((9H-fluoren-9-yl)methoxy)carbonyl)amino)acetamido)-6-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-6-oxohexyl)carbamate (INX A6-1)

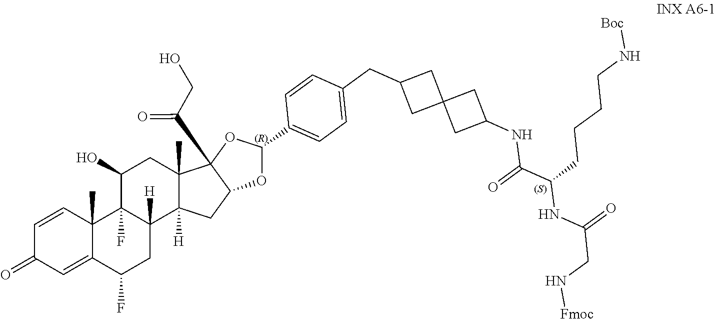

A 25 mL single-necked round bottom flash was charged with N2-((((9H-fluoren-9-yl)methoxy)carbonyl) glycyl)-N6-(tert-butoxycarbonyl)-L-lysine (INX—W-2) (0.25 g, 0.48 mmol), HATU (0.27 g, 0.72 mmol) and DMF (3 mL). To this solution, DIPEA (0.12 g, 0.96 mmol) and (2S,6aS,6bR,7S,8aS,8bS,10R,11aR,12aS,12bS)-10-(4-((6-aminospiro[3.3]heptan-2-yl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-25) (0.3 g, 0.48 mmol) were added to the reaction mixture and stirred for 1 h. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile:water, 50:50) to give title compound as light yellow Solid (0.28 g, 51.46%). LCMS: 1131.7 [M+H]⁺.

### Synthesis of tert-butyl ((S)-5-(2-aminoacetamido)-6-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-6-oxohexyl)carbamate (INX A6-2)

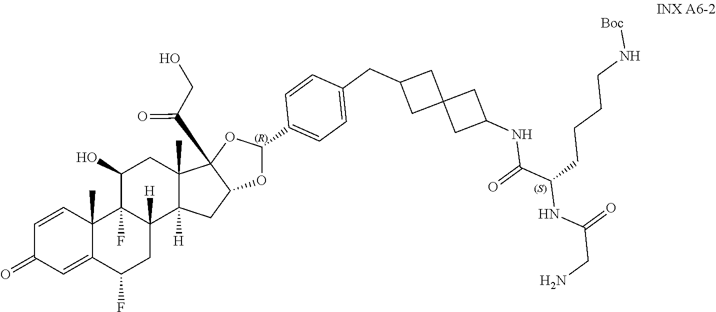

### Procedure

A 25 mL single-necked round bottom flash was charged with tert-butyl ((S)-5-(2-((((9H-fluoren-9-yl)methoxy)carbonyl)amino)acetamido)-6-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-6-oxohexyl)carbamate (INX A6-1) (0.25 g, 0.22 mmol) and THF (3 mL). To this solution, diethylamine (0.16 g, 2.0 mmol) was added at room temperature and stirred for 3 h. After completion of reaction as indicated by TLC, reaction mixture was evaporated under vacuum and triturated with diethyl ether to give title compound as yellow solid (0.19 g, 94.21%). LCMS: 909.5 [M+H]⁺.

### Synthesis of tert-butyl ((S)-5-(2-(2-bromoacetamido)acetamido)-6-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-6-oxohexyl)carbamate (INX A6-3)

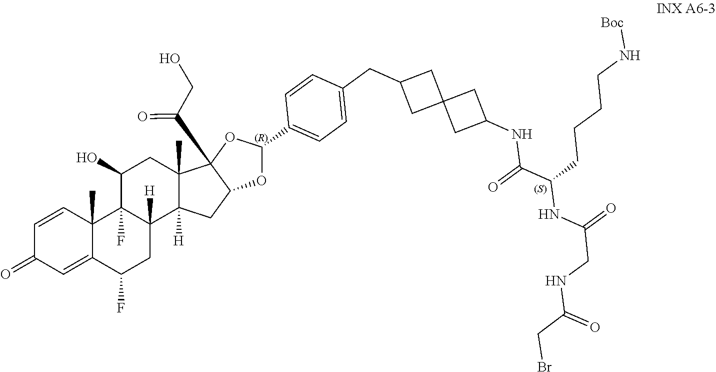

### Procedure

A 10 mL single-necked round bottom flash was charged with tert-butyl ((S)-5-(2-aminoacetamido)-6-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-6-oxohexyl)carbamate (INX A6-2) (0.19 g, 0.21 mmol) and DCM (3 mL). To this solution, Na₂CO₃ (0.093 g, 0.83 mmol) dissolved in water (1 mL) followed by bromoacetyl bromide (0.088 g, 0.41 mmol) were added and stirred at room temperature for 1 h. After completion of reaction as indicated by TLC, reaction mixture was quenched with water and extracted with DCM. The combined organic layer was dried over Na₂SO₄ and evaporated under vacuum. The crude was purified by reverse phase column chromatography (acetonitrile:water, 50:50) to give title compound as light yellow solid (0.090 g, 41.81%). LCMS: 1029.7 [M+H]⁺.

### Synthesis of (S)-6-amino-2-(2-(2-bromoacetamido)acetamido)-N-(6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl) hexanamide 2,2,2-trifluoroacetate (INX A6)

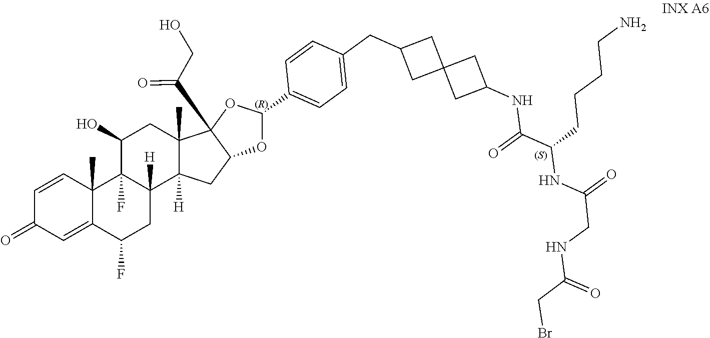

### Procedure

A 10 mL single-necked round bottom flash was charged with of tert-butyl ((S)-5-(2-(2-bromoacetamido)acetamido)-6-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-6-oxohexyl)carbamate (INX A6-3) (0.070 g, 0.067 mmol) in DCM (4 mL). To this solution, TFA (0.038 g, 0.33 mmol) was added at room temperature and stirred for 2 h at room temperature. After completion of reaction as indicated by TLC, reaction mixture was evaporated under vacuum. The crude was purified by prep-HPLC (Column: YMC-Actus Triart Prep C18-S, 250×20 mm S-5 μm, 12 nm, Mobile phase: A=0.05% TFA in water, B=acetonitrile, A:B=50:50), retention time 16 min) to give title compound as an off white solid (0.005 g, 7.05%). LCMS: 929.59 [M+H]⁺; ¹H NMR (400 MHZ, MeOD): 0 7.36-7.34 (m, 3H), 7.16 (d, J=8.0 Hz, 2H), 6.38-6.34 (m, 2H), 5.67-5.49 (m, CHF, J=48.8 Hz, 1H) 5.49 (s, Acetal-H, 1H), 5.08 (d, J=3.6 Hz, C16H, 1H), 4.65 (d, J=19.6 Hz, 1H), 4.37-4.32 (m, 3H), 4.20-4.10 (m, 2H), 3.97 (s, 2H), 3.94 (d, 2H), 3.00-2.90 (m, 2H), 2.80-2.60 (m, 2H), 2.50-1.55 (m, 20H), 1.60 (s, 3H), 1.55-1.40 (m, 2H), 1.00 (s, 3H).

### Synthesis of (S)-2-(2-(2-bromoacetamido)acetamido)-N1-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)succinamide (INX-A11)

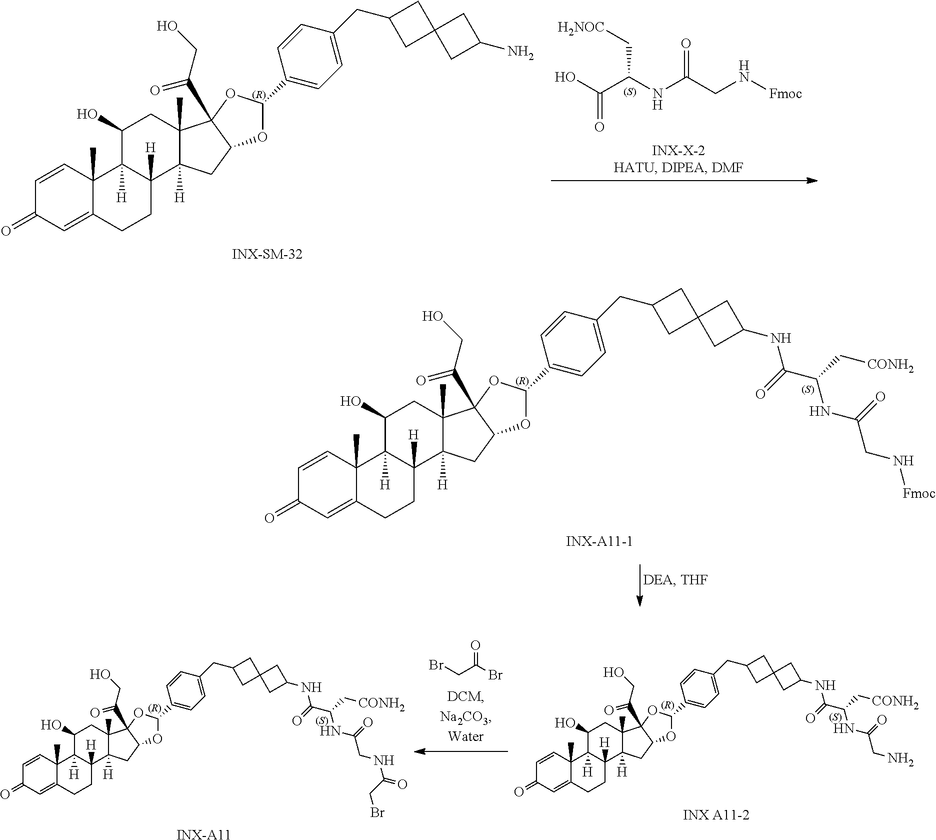

### Synthesis of (9H-fluoren-9-yl) methyl (2-(((S)-4-amino-1-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-1,4-dioxobutan-2-yl)amino)-2-oxoethyl)carbamate (INX-A11-1)

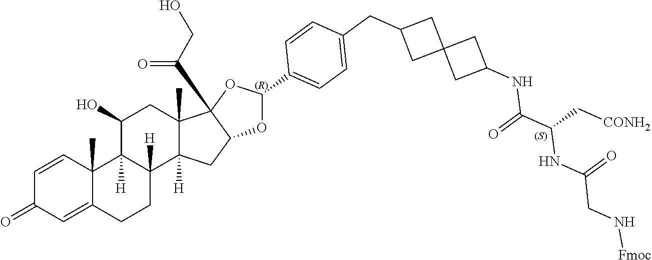

A 25 mL single-necked round bottom flash was charged with (6aR,6bS,7S,8aS,8bS,10R,11aR,12aS,12bS)-10-(4-((6-aminospiro[3.3]heptan-2-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-32) (0.50 g, 0.85 mmol), (((9H-fluoren-9-yl)methoxy)carbonyl) glycyl-L-asparagine (INX—X-2) (0.52 g, 1.27 mmol) and DMF (5 mL). To this solution, HATU (0.64 g, 1.70 mmol) and DIPEA (0.32 g, 2.55 mmol) were added at room temperature and stirred for 16 h. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile:water, 50:50) to give title compound as light yellow solid (0.32 g, 38.34%). LCMS: 981.5 [M+H]⁺.

### Synthesis of (S)-2-(2-aminoacetamido)-N1-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl) succinamide (INX-A11-2)

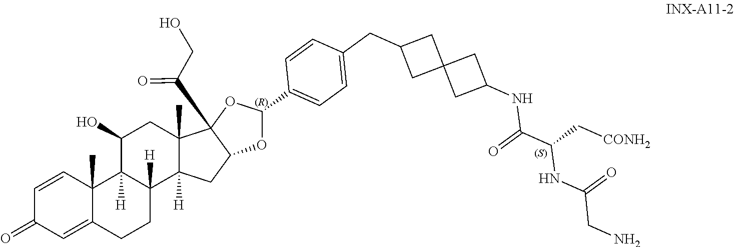

### Procedure

A 10 mL single-necked round bottom flash was charged with (9H-fluoren-9-yl) methyl (2-(((S)-4-amino-1-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-1,4-dioxobutan-2-yl)amino)-2-oxoethyl)carbamate (INX-A11-1) (0.32 g, 0.32 mmol) and THF (5 mL). To this solution, diethyl amine (0.23 g, 3.23 mmol) was added at room temperature and stirred for 2 h. After completion of reaction as indicated by TLC, reaction mixture was evaporated under vacuum and triturated with diethyl ether to give title compound as yellow solid (0.20 g, 82.35%). LCMS: 759.5 [M+H]⁺.

### Synthesis of (S)-2-(2-(2-bromoacetamido)acetamido)-N1-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl) succinamide (INX-A11)

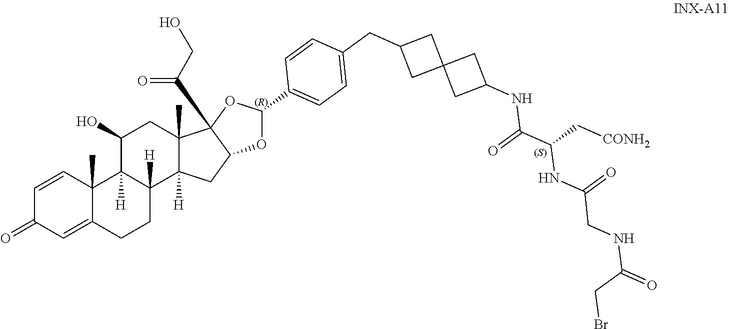

### Procedure

A 25 mL single-necked round bottom flash was charged with(S)-2-(2-aminoacetamido)-N1-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl) succinamide (INX-A11-2) (0.1 g, 0.13 mmol) and DCM (3 mL). To this solution, Na₂CO₃ (0.055 g, 0.52 mmol) dissolved in water (0.5 mL) followed by bromoacetyl bromide (0.053 g, 0.26 mmol) were added at room temperature and stirred for 1 h. After completion of reaction as indicated by TLC, reaction mixture was quenched with water and extracted with DCM. The combined organic layer was dried over Na₂SO₄ and evaporated under vacuum. The crude title compound was purified by prep HPLC

(Column: YMC C18 120 gm, 50 μm, Mobile phase: A=0.05% TFA in water, B=acetonitrile; A:B, 55:45), retention time 15.51 min) to give title compound as white solid (0.035 g, 30.5%). LCMS: calculated for C₄₄H₅₆⁷⁹BrFN₄O₁₀ (879.32), found 879.4 [M+H]⁺.

### Synthesis of S-(2-((2-(((S)-4-amino-1-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-1,4-dioxobutan-2-yl)amino)-2-oxoethyl)amino)-2-oxoethyl)-L-cysteine compound with 2,2,2-trifluoroacetic acid (INX-A11-CYS)

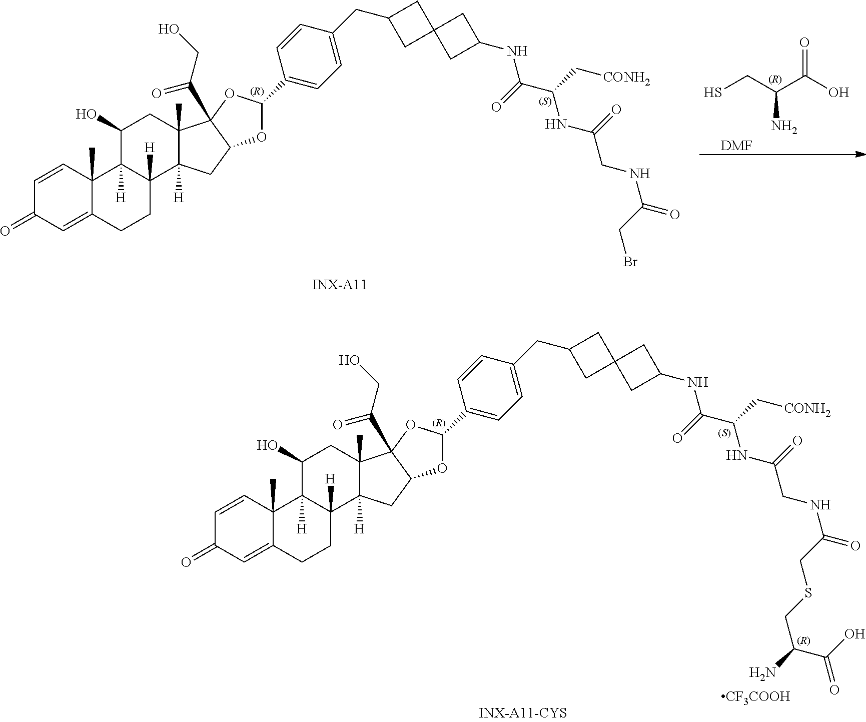

### Procedure

A 10 mL single-necked round bottom flash was charged with(S)-2-(2-(2-bromoacetamido)acetamido)-N1-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl) succinamide (INX-A11) (0.1 g, 0.11 mmol) and DMF (1 mL). To this solution, L-cysteine (0.026 g, 0.22 mmol) was added and stirred for 16 h at room temperature. After completion of reaction as indicated by LCMS, reaction mixture was lyophilized and the crude was purified by prep HPLC (Column: SUNFIRE Prep C18 OBD, 19×250 mm, 5 μm, Mobile phase: A=0.05% TFA in water, B=acetonitrile: MTBE, 90:10, A:B=60:40), retention time 16 min) to give title compound as off white solid (0.0096 g, 9.61%). LCMS: 920.5 [M+H]⁺; ¹H NMR (400 MHZ, MeOD): 8.54 (t, 1H), 8.34 (d, 1H), 7.88 (br d, 3H), 7.43 (br s, 1H), 7.35-7.31 (m, 3H), 7.15 (d, J=8.0 Hz, 2H), 6.87 (br s, 1H), 6.17 (d, J=10 Hz 1H), 5.94 (s, 1H), 5.39 (s, Acetal-H, 1H), 5.10 (br s, 1H), 4.93 (d, J=5.2 Hz, C16H, 1H), 4.80 (br s, 1H), 4.53-4.43 (m, 2H), 4.30-4.16 (m, 2H), 4.01-3.98 (m, 1H), 3.71 (d, 2H), 2.90-3.00 (m, 2H), 2.70-1.60 (m, 25H), 1.40 (s, 3H), 1.02-0.95 (m, 2H), 0.87 (s, 3H).

### Synthesis of (6aR,6bS,7S,8aS,8bS,10R,11aR,12aS,12bS)-10-(4-((3-aminospiro[3.3]heptan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-45)

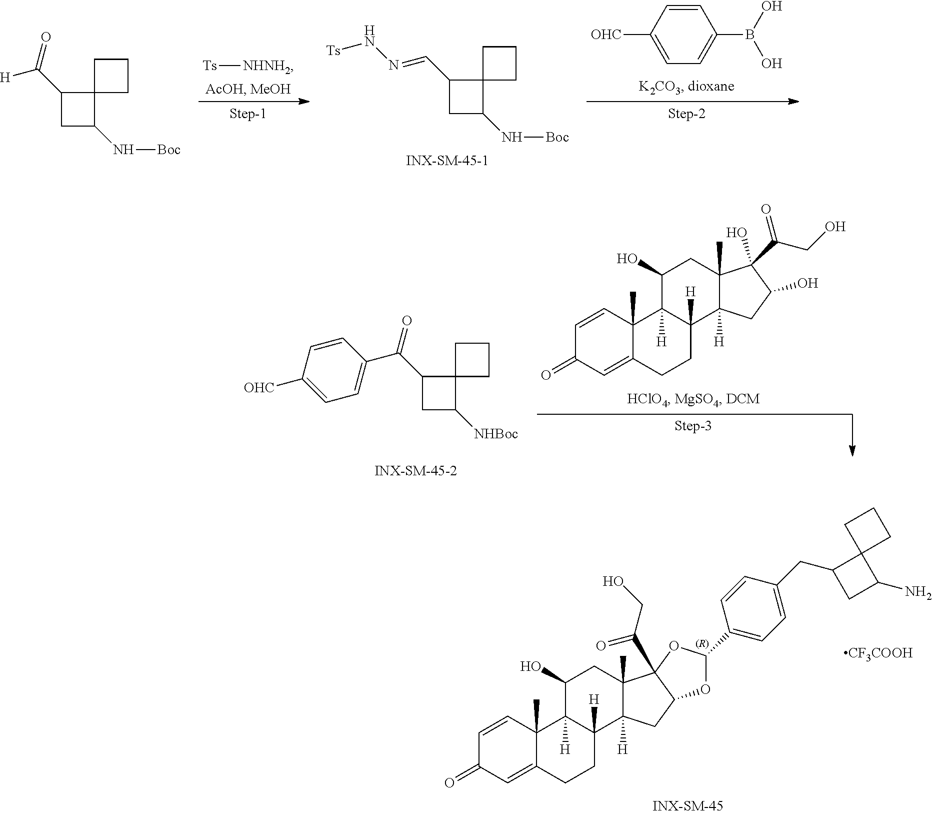

### Synthesis of tert-butyl (E)-(3-((2-tosylhydrazono)methyl)spiro[3.3]heptan-1-yl)carbamate (INX-SM-45-1)

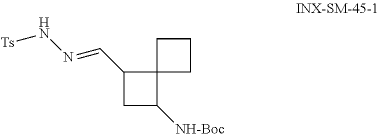

### Procedure

A 50 mL single-necked round bottom flash was charged tert-butyl (3-formylspiro[3.3]heptan-1-yl)carbamate (1.0 g, 4.18 mmol) and EtOH (25 mL) under nitrogen. To this solution, p-toluenesulfonhydrazide (0.934 g, 6.276 mmol) was added and catalytic amount of AcOH (0.2 mL) and stirred for 0.5h at room temperature. After completion of reaction as indicated by TLC, reaction mixture was poured into water and solid was formed and filter it. The compound dry by under vacuum to give title compound as white solid (0.81 g, 46.94%). LCMS: 407.53 [M+H]⁺

### Synthesis of tert-butyl (3-(4-formylbenzyl) spiro[3.3]heptan-1-yl)carbamate (INX-SM-45-2)

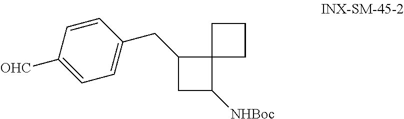 [SMILES: CC(C)(C)OC(=O)NC1CC(Cc2ccc(C=O)cc2)C12CCC2]

### Procedure

A 35 mL vial with tert-butyl (E)-(3-((2-tosylhydrazono)methyl)spiro[3.3]heptan-1-yl)carbamate (INX-SM-45-1) (0.5 g, 1.22 mmol) and dioxane (20 mL) under nitrogen. To this solution, (4-formylphenyl)boronic acid (0.551 g, 3.68 mmol) and K₂CO3 (0.508 g, 3.68 mmol) was added at room temperature and stirred for another 2 h at 100° C. After completion of reaction as indicated by TLC, reaction mixture was poured into water and extracted with ethyl acetate. The combined organic layer was dried over Na₂SO₄ and evaporated under vacuum to give crude product. The crude was purified by silica gel column chromatography (ethyl acetate/hexane, 30:70) to give title compound as yellow sticky solid (0.180 g, 44.7%). LCMS: 330 [M+H]⁺.

### Synthesis of (6aR,6bS,7S,8aS,8bS,10R,11aR,12aS,12bS)-10-(4-((3-aminospiro[3.3]heptan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-45)

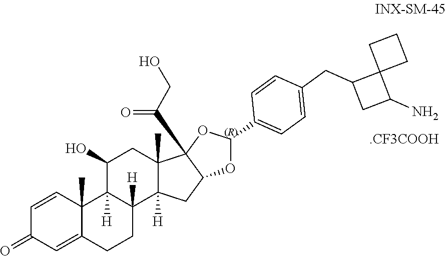

### Procedure

A 50 mL single-necked round bottom flash was charged with tert-butyl (3-(4-formylbenzyl) spiro[3.3]heptan-1-yl)carbamate (INX-SM-45-2) (0.400 g, 1.244 mmol) and (8S,9S,10R,11S,13S,14S,16R,17S)-11,16,17-trihydroxy-17-(2-hydroxyacetyl)-10, 13-dimethyl-6,7,8,9,10,11,12,13,14,15, 16,17-dodecahydro-3H-cyclopenta[a]phenanthren-3-one (16-alpha-hydroxyprednisolone) (0.457 g, 1.244 mmol). It was dissolved in DCM (8 mL) and MgSO₄ (0.730 g, 6.07 mmol) was added into the solution. To this solution, HClO₄ (0.405 g, 6.07 mmol) was added and stirred for another 2 h at room temperature. After completion of reaction as indicated by TLC, reaction mixture was quenched by sat. NaHCO₃ solution and concentrated over vacuum. Then crude was triturate with cold water and participated was filtered and dried under vacuum to give crude. The portion of crude (0.10 g) was purified by prep-HPLC (Column: C18 (250*21.2) MM, 5 μm, Mobile phase: A=0.05% TFA in water, B=acetonitrile, A:B=53:46, retention time 12 min) to give title compound (0.013 g, 7%). LCMS: 588.4 [M+H]⁺; ¹H NMR (400 MHZ, MeOD): 0 7.47 (d, J=10.0 Hz, 1H), 7.40 (d, J=8.0 Hz, 2H), 7.28 (d, J=8.0 Hz, 2H), 6.27 (dd, J=10.0 & 1.6 Hz 1H), 6.04 (s, 1H), 5.49 (s, Acetal-H, 1H), 5.08 (d, J=4.8 Hz, C16H, 1H), 4.61 (d, J=19.2 Hz, 1H), 4.44-4.43 (m, 1H), 4.34 (d, J=19.2 Hz, 1H), 3.05-2.95 (m, 1H), 2.80-1.60 (m, 20H), 1.52 (s, 3H), 1.20-1.00 (m, 2H), 1.01 (s, 3H).

### Synthesis of (4S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-1-yl)amino)-5-oxopentanoic acid (INX-A8)

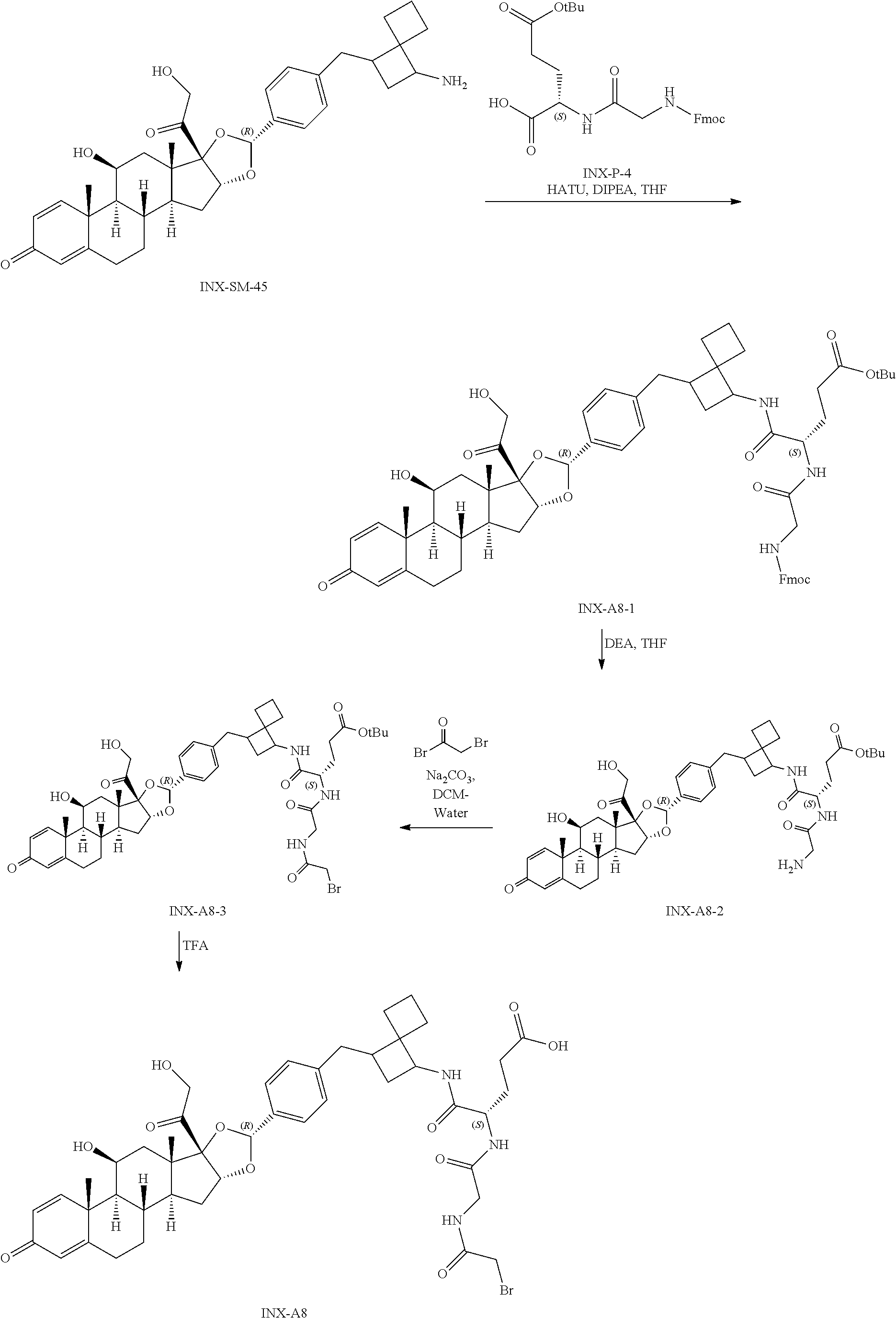

### Synthesis of tert-butyl (4S)-4-(2-((((9H-fluoren-9-yl)methoxy)carbonyl)amino)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-1-yl)amino)-5-oxopentanoate (INX-A8-1)

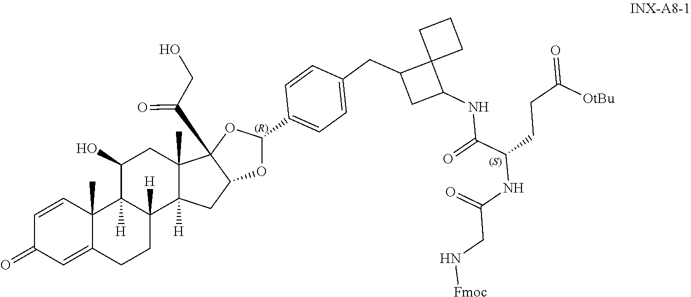

### Procedure

A 25 mL single-necked round bottom flash was charged(S)-2-(2-((((9H-fluoren-9-yl)methoxy)carbonyl)amino)acetamido)-5-(tert-butoxy)-5-oxopentanoic acid (INX-P-4) (0.410 g, 0.850 mmol) and HATU (0.485 g, 1.27 mmol) in DMF (5 mL) was added DIPEA (0.329 g, 2.55 room mmol) at temperature. To this solution, tert (6aR,6bS,7S,8aS,8bS,10R,11aR,12aS,12bS)-10-(4-((3-aminospiro[3.3]heptan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-45) (0.5 g, 0.850 mmol) was added at room temperature and stirred for 3 h at room temperature. After completion of reaction as indicated by TLC, reaction mixture was poured into water and extracted with ethyl acetate. The combined organic layer was dried over Na₂SO₄ and evaporated under vacuum to give crude product. The crude was purified by Normal phase column chromatography (DCM: MeOH, 90:10) to give title compound as light yellow solid (0.3 g, 33.51%). LCMS: 1052.9 [M+H]⁺

### Synthesis of tert-butyl (4S)-4-(2-aminoacetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-1-yl)amino)-5-oxopentanoate (INX-A8-2)

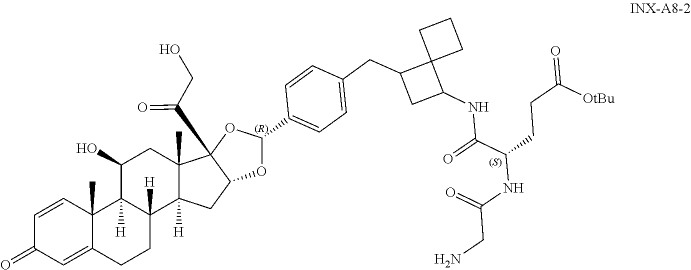

### Procedure

A 25 mL single-necked round bottom flash was charged with tert-butyl (4S)-4-(2-((((9H-fluoren-9-yl)methoxy)carbonyl)amino)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-1-yl)amino)-5-oxopentanoate (INX-A8-1) (0.3 g, 0.285 mmol) in THF (5 mL). To this solution, diethylamine (0.208 g, 2.85 mmol) was added at room temperature and stirred for 2.5h at room temperature. After completion of reaction as indicated by TLC, reaction mixture was directly concentrated over vacuum to afford crude and purified by trituration with DCM and hexane and dried under vacuum to give title compound as white solid (0.175 g, 74%). LCMS: 831 [M+H]⁺.

### Synthesis of tert-butyl (4S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-1-yl)amino)-5-oxopentanoate (INX-A8-3)

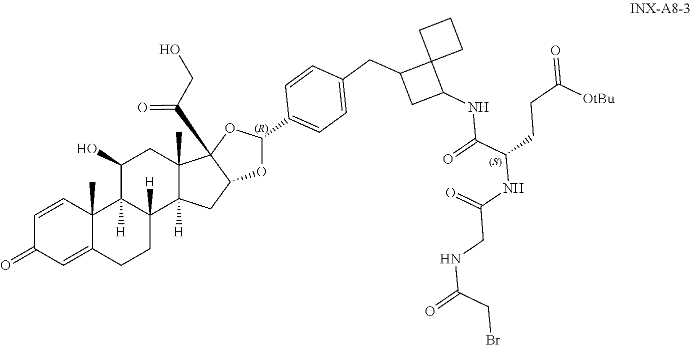

### Procedure

A 25 mL single-necked round bottom flash was charged tert-butyl (4S)-4-(2-aminoacetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-1-yl)amino)-5-oxopentanoate (INX-A8-2) (0.175 g, 0.210 mmol) in DCM (5 mL). To this solution, Na₂CO₃ (0.044 g, 0.421 mmol) at room temperature which is dissolve in water (1 ml) than to it bromoacetyl bromide (0.042 g, 0.210 mmol) was added at room temperature and stirred for 1.5h. After completion of reaction as indicated by TLC, reaction mixture was quenched with water and extracted with MDC. The combined organic layer was dried over Na₂SO₄ and evaporated under vacuum to give crude product. The crude was purified by reverse phase column chromatography (acetonitrile/water: 50:50) to give title compound as off white solid (0.055 g, 27.43%). LCMS: calculated for C₄₉H₆₅⁷⁹BrFN₃O₁₁ (950.38), found 950.6 [M+H]⁺.

### Synthesis of (4S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-1-yl)amino)-5-oxopentanoic acid (INX-A8)

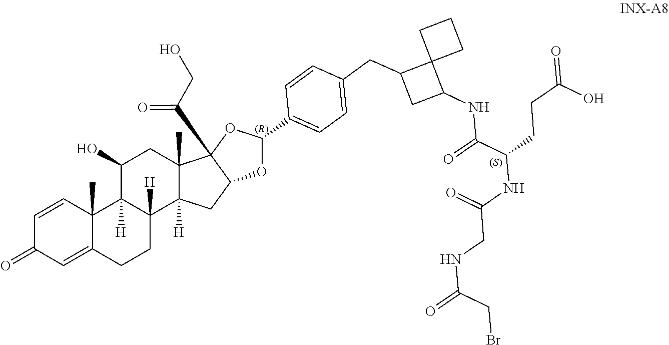

### Procedure

A 25 mL single-necked round bottom flash was charged with of tert-butyl (4S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-1-yl)amino)-5-oxopentanoate (INX-A8-3) (0.250 g, 0.262 mmol) and DCM (15 mL). To this solution, TFA (2.5 mL) was added at room temperature and stirred for 2 h at room temperature. After completion of reaction as indicated by TLC, reaction mixture was evaporated under vacuum. The crude was purified by prep-HPLC (Column: C18, 250*21.2 mm, 5 μm, Mobile phase: A=0.05% trifluroacetic acid in water, B=acetonitrile, A:B=65:25, retention time 4 min) to give title compound as off white solid (0.014 g, 5.95%). LCMS: 894.4 [M+H]⁺; ¹H NMR (400 MHZ, MeOD): δ: 7.47 (d, J=10.0 Hz, 1H), 7.37 (d, J=8.4 Hz, 2H), 7.25 (d, J=8.0 Hz, 2H), 6.27 (dd, J=10.0 & 1.6 Hz 1H), 6.04 (s, 1H), 5.46 (s, Acetal-H, 1H), 5.07 (d, J=5.2 Hz, C16H, 1H), 4.63 (d, J=19.6 Hz, 1H), 4.45-4.43 (m, 2H), 4.34 (d, J=19.6 Hz, 1H), 4.10-3.90 (m, 5H), 2.90-1.60 (m, 24H), 1.52 (s, 3H), 1.25-1.05 (m, 2H), 1.00 (s, 3H).

### Synthesis of (6aR,6bS,7S,8aS,8bS,10R,11aR,12aS,12bS)-10-(4-((6-aminospiro[2.5]octan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-46)

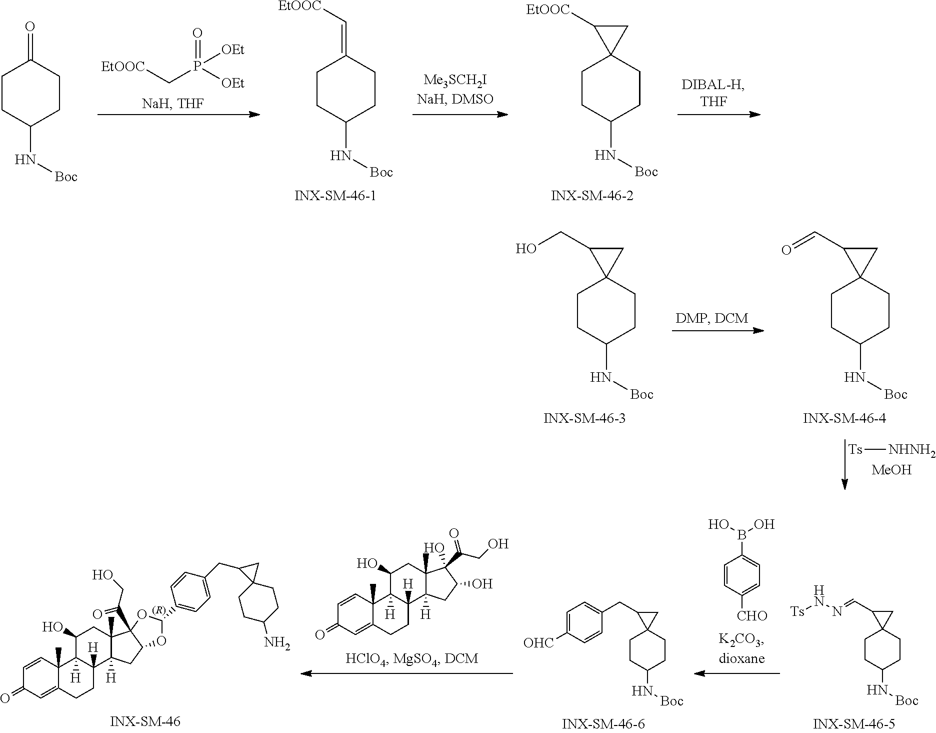

### Synthesis of ethyl 2-(4-((tert-butoxycarbonyl)amino)cyclohexylidene)acetate (INX-SM-46-1)

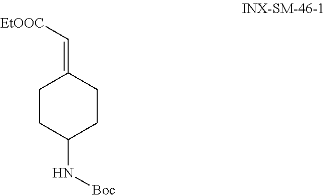 [SMILES: CCOC(=O)C=C1CCC(NC(=O)OC(C)(C)C)CC1]

### Procedure

A 100 mL single-necked round bottom flask was charged with NaH (1.2 g, 30.5 mmol) and THF (50 mL). To this solution, ethyl 2-(diethoxyphosphoryl) acetate (6.83 g, 30.5 mmol) was added at 0° C. and stirred at room temperature for 30 min. Solution of tert-butyl (4-oxocyclohexyl)carbamate (5.0 g, 23.47 mmol) in THF (5 mL) was added to the reaction mixture and stirred at room temperature for 12 hr. After completion of reaction as indicated by TLC, reaction mixture was quenched with saturated solution of NH₄Cl (100 mL) and extracted with ethyl acetate. The combined organic layer was dried over Na₂SO₄ and evaporated under vacuum to give title compound as white solid. (6.5 g, 97.78%). LCMS: 284.1 [M+H]⁺.

### Synthesis of ethyl 6-((tert-butoxycarbonyl)amino) spiro[2.5]octane-1-carboxylate (INX-SM-46-2)

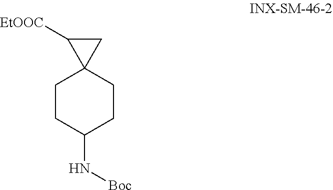 [SMILES: CCOC(=O)C1CC12CCC(NC(=O)OC(C)(C)C)CC2]

### Procedure

A 100 mL single-necked round bottom flask was charged with 60% NaH (0.7 g, 17.6 mmol) and DMSO (40 mL). To this solution, Trimethyl sulfoxonium iodide (3.8 g, 17.6 mmol) was added at 0° C. and stirred at room temperature for 30 min. Solution of ethyl 2-(4-((tert-butoxycarbonyl)amino)cyclohexylidene)acetate (INX-SM-46-1) (2.0 g, 7.06 mmol) in DMSO (5 mL) was added to this solution and allowed to stir at room temperature for 12 hr. After completion of reaction as indicated by TLC, reaction mixture was quenched with saturated solution of NH₄Cl (100 mL) and extracted with ethyl acetate. The combined organic layer was dried over Na₂SO₄ and evaporated under vacuum. The crude was purified by silica gel column chromatography (ethyl acetate:hexane, 20:80) to give title compound as off white solid (0.75 g, 35.73%). LCMS 298.2 [M+H]⁺.

### Synthesis of tert-butyl (1-(hydroxymethyl)spiro[2.5]octan-6-yl)carbamate (INX-SM-46-3)

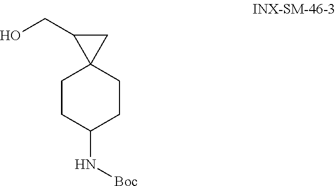 [SMILES: CC(C)(C)OC(=O)NC1CCC2(CC1)CC2CO]

### Procedure

A 35 mL glass vial was charged with ethyl 6-((tert-butoxycarbonyl)amino) spiro[2.5]octane-1-carboxylate (INX-SM-46-2) (0.1 g, 0.33 mmol) and THF (5 mL) under nitrogen. To this solution, LiBH₄ (3.3 mL, 67.3 mmol) was added at 0° C. and stirred at room temperature for 12 h. After completion of reaction as indicated by TLC, reaction mixture was quenched with dil. HCl (30 mL) and extracted with ethyl acetate. The combined organic layer was dried over Na₂SO₄ and evaporated under vacuum. The crude was purified by silica gel column chromatography (ethyl acetate/hexane, 50:50) to give title compound as colorless liquid (0.05 g, 58.22%). LCMS: 256.2 [M+H]⁺.

### Synthesis of tert-butyl (1-formylspiro[2.5]octan-6-yl)carbamate (INX-SM-46-4)

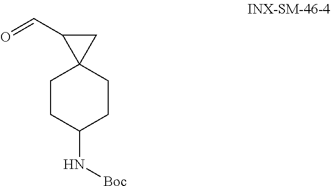 [SMILES: CC(C)(C)OC(=O)NC1CCC2(CC1)CC2C=O]

A 25 mL single-necked round bottom flask was charged with tert-butyl (1-(hydroxymethyl)spiro[2.5]octan-6-yl)carbamate (INX-SM-46-3) (0.2 g, 0.78 mmol) and THF (5 mL). To this solution, DMP (0.498 g, 1.17 mmol) was added at room temperature and stirred for 2 h. After completion of reaction as indicated by TLC, reaction mixture was quenched with NaHCO₃ solution and extracted with ethyl acetate. The combined organic layer was washed with brine, dried over Na₂SO₄ and evaporated under vacuum to give title compound as yellow liquid (0.2 g, crude) and it was forwarded as such for the next step without further purification.

### Synthesis of tert-butyl (E)-(1-((2-tosylhydrazono)methyl)spiro[2.5]octan-6-yl)carbamate (INX-SM-46-5)

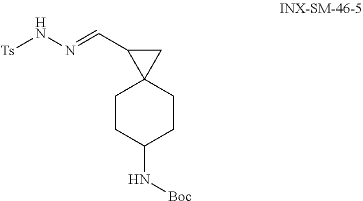 [SMILES: Cc1ccc(S(=O)(=O)N/N=C/C2CC23CCC(NC(=O)OC(C)(C)C)CC3)cc1]

### Procedure

A 10 mL glass vial was charged with tert-butyl (1-formylspiro[2.5]octan-6-yl)carbamate (INX-SM-46-4) (0.85 g, 3.35 mmol) and ethanol (20 mL). To this solution, p-toluenesulfonyl hydrazide (0.74 g, 4.03 mmol) and acetic acid (0.05 g, 0.88 mmol) was added and stirred for 1 h at room temperature. After completion of reaction as indicated by TLC, reaction mixture was poured into water and the solid was filtered and dried under vacuum to give title compound as white solid (1.0 g, 70.70%). LCMS: 422.3 [M+H]⁺

### Synthesis of tert-butyl (1-(4-formylbenzyl) spiro[2.5]octan-6-yl)carbamate (INX-SM-46-6)

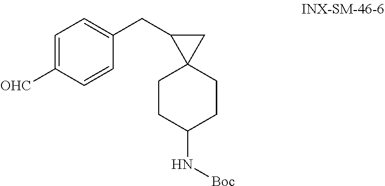 [SMILES: CC(C)(C)OC(=O)NC1CCC2(CC1)CC2Cc1ccc(C=O)cc1]

### Procedure

A 50 mL single-necked round bottom flask was charged with tert-butyl (E)-(1-((2-tosylhydrazono)methyl)spiro[2.5]octan-6-yl)carbamate (INX-SM-46-5) (0.5 g, 1.18 mmol), K2CO3 (0.24 g, 1.78 mmol) and dioxane (15 mL) was added and the mixture was degassed with N2 for 30 min. To this solution, (4-formylphenyl)boronic acid (0.26 g, 1.78 mmol) was added and heated at 110° C. for 2 h. After completion of reaction as indicated by TLC, reaction mixture was poured into water (50 mL) and extracted with ethyl acetate. The combined organic layer was dried over Na₂SO₄ and evaporated under vacuum. The crude was purified by silica gel column chromatography (ethyl acetate:hexane, 1:1) to give title compound as colorless liquid (0.27 g, 67.5%). LCMS: 344.21 [M+H]⁺

### Synthesis of (6aR,6bS,7S,8aS,8bS,10R,11aR,12aS,12bS)-10-(4-((6-aminospiro[2.5]octan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-46)

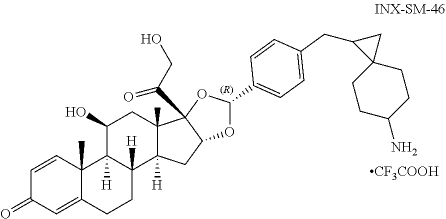

### Procedure

A 35 mL glass vial was charged with tert-butyl (1-(4-formylbenzyl) spiro[2.5]octan-6-yl)carbamate (INX-SM-46-6) (0.55 g, 1.60 mmol), (8S,9S,10R,11S,13S,14S,16R,17S)-11, 16, 17-trihydroxy-17-(2-hydroxyacetyl)-10, 13-dimethyl-6,7, 8, 9, 10, 11, 12, 13, 14, 15, 16, 17-dodecahydro-3H-cyclopenta[a]phenanthren-3-one (16-α-hydroxyprednisolone) (0.602 g, 1.60 mmol) and DCM (5 mL). To this solution, MgSO₄ (0.96 g, 8.01 mmol) and HClO₄ (0.80 g, 8.01 mmol) were added and stirred for 1 h at room temperature. After completion of reaction as indicated by TLC, reaction mixture was quenched with saturated sodium bicarbonate solution and extracted with ethyl acetate. The combined organic layer was dried over Na₂SO₄ and evaporated under vacuum. The portion of the crude (0.150 g) was purified by prep-HPLC (Column: C18-(250×21.2 mm), 5 MICRON, Mobile phase: A=0.05% TFA in water, B=acetonitrile: methanol: IPA (65:25:10); A:B, 75:25), retention time 13.67 min) to give title compound (0.005 g, 2.8%). LCMS: 602.4 [M+H]⁺;

¹H NMR (400 MHZ, MeOD): δ: 7.47 (d, J=10.0 Hz, 1H), 7.39 (d, J=8.0 Hz, 2H), 7.31 (d, J=8.0 Hz, 2H), 6.27 (dd, J=10.0 & 2.4 Hz 1H), 6.04 (s, 1H), 5.47 (s, Acetal-H, 1H), 5.07 (d, J=5.2 Hz, C16H, 1H), 4.70-4.60 (m, 2H), 4.45 (br s, 1H), 4.35 (d, J=19.2 Hz, 1H), 3.00-0.80 (m, 22H), 1.51 (s, 3H), 1.00 (s, 3H), 00.59-0.56 (m, 1H), 26-0.25 (m, 1H).

### Synthesis of (4S)-4-(2-(2-bromoacetamido)acetamido)-5-((1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[2.5]octan-6-yl)amino)-5-oxopentanoic acid (INX-A9)

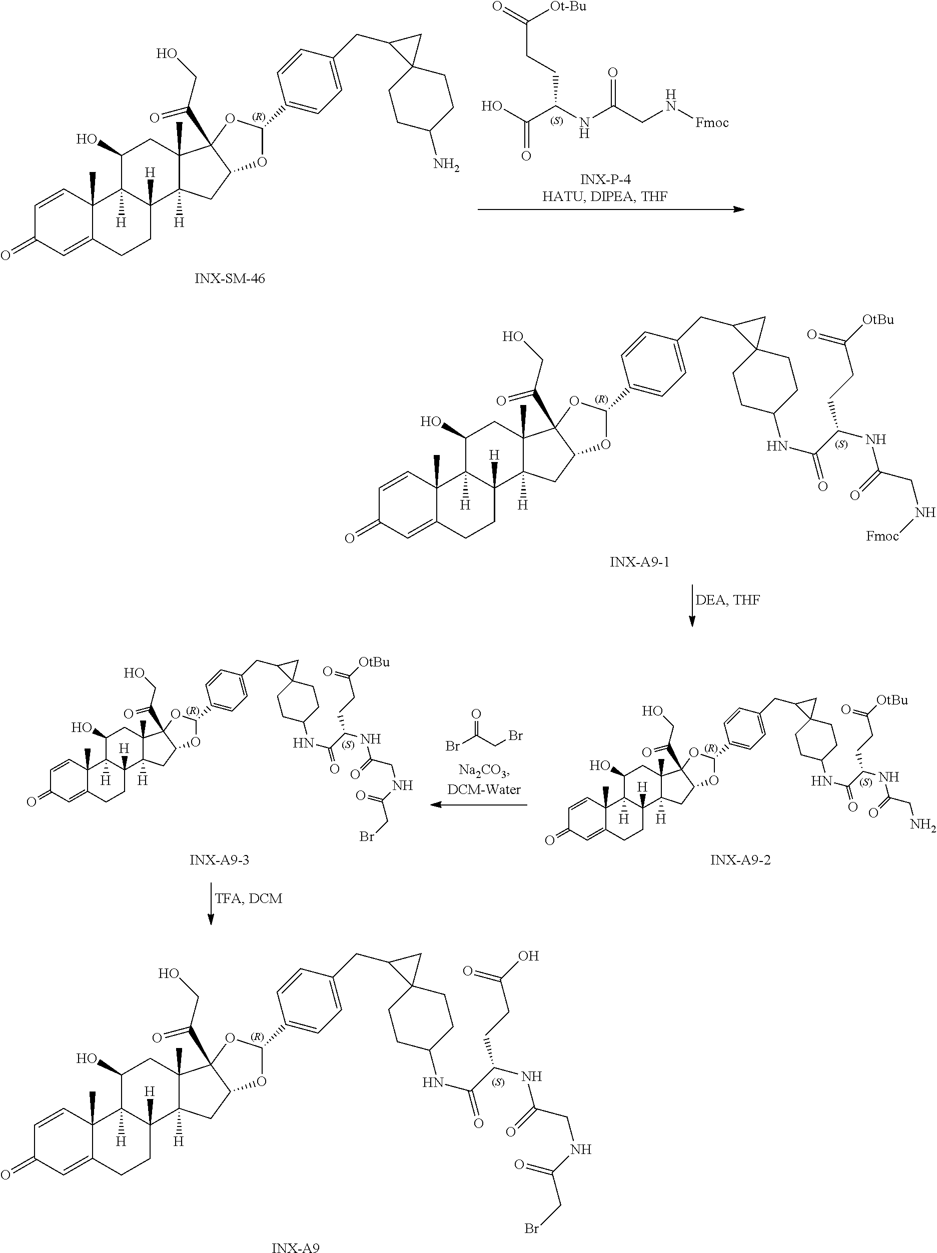

### Synthesis of tert-butyl (4S)-4-(2-((((9H-fluoren-9-yl)methoxy)carbonyl)amino)acetamido)-5-((1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[2.5]octan-6-yl)amino)-5-oxopentanoate (INX-A9-1)

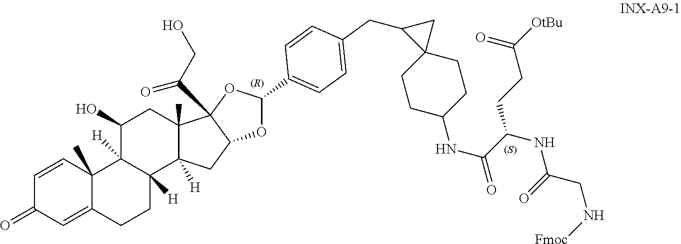

### Procedure

A 10 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) (0.32 g, 0.68 mmol), ((6aR,6bS,7S,8aS,8bS,10R,11aR,12aS,12bS)-10-(4-((6-aminospiro[2.5]octan-1-yl)methyl)phenyl)-7-hydroxy-8b-(2-hydroxyacetyl)-6a,8a-dimethyl-1, 2, 6a,6b,7,8,8a,8b,11a,12,12a,12b-dodecahydro-4H-naphtho[2′,1′:4,5]indeno[1,2-d][1,3]dioxol-4-one (INX-SM-46) (0.4 g, 0.68 mmol) and DMF (5 mL). To this solution, DIPEA (0.13 g, 1.02 mmol) and HATU (0.33 g, 1.02 mmol) were added and stirred for 1 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water, 50:50) to give title compound as pale yellow solid (0.3 g, 42.31%). LCMS: 1066.41 [M+H]⁺.

### Synthesis of tert-butyl (4S)-4-(2-aminoacetamido)-5-((1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[2.5]octan-6-yl)amino)-5-oxopentanoate (INX-A9-2)

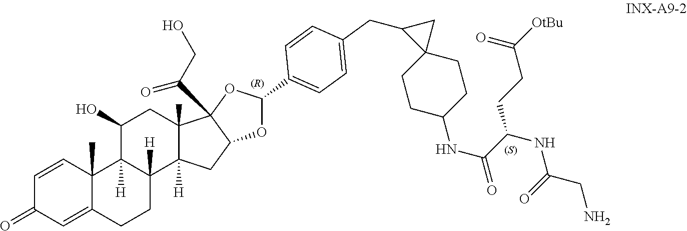

### Procedure

A 10 mL glass vial was charged with tert-butyl (4S)-4-(2-((((9H-fluoren-9-yl)methoxy)carbonyl)amino)acetamido)-5-((1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[2.5]octan-6-yl)amino)-5-oxopentanoate (INX-A9-1) (0.3 g, 0.28 mmol) and THF (3 mL). To this solution, diethyl amine (0.20 g, 2.81 mmol) was added at room temperature and stirred for 3 h. After completion of reaction as indicated by TLC, reaction mixture was evaporated under vacuum and triturated with diethyl ether and pentane to give title compound as yellow solid (0.16 g, 67.40%) LCMS: 844.5 [M+H]⁺.

### Synthesis of tert-butyl (4S)-4-(2-(2-bromoacetamido)acetamido)-5-((1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[2.5]octan-6-yl)amino)-5-oxopentanoate (INX-A9-3)

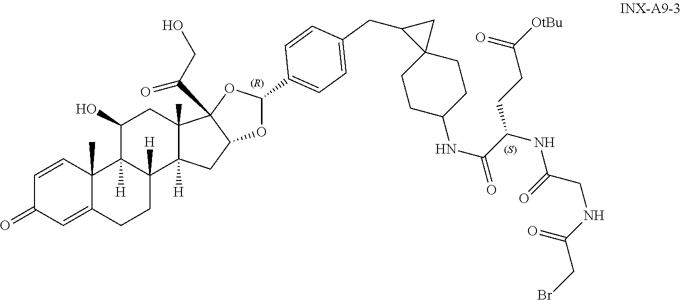

### Procedure

A 25 mL single-necked round bottom flash was charged with tert-butyl (4S)-4-(2-aminoacetamido)-5-((1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[2.5]octan-6-yl)amino)-5-oxopentanoate (INX-A9-2) (0.16 g, 0.18 mmol) and DCM (10 mL). To this solution, Na₂CO₃ (0.040 g, 0.37 mmol) dissolved in water (1 mL) and bromoacetyl bromide (0.038 g, 0.18 mmol) were added and stirred for 1 h at room temperature. After completion of reaction as indicated by TLC, reaction mixture was quenched with water and extracted with 10% methanol in DCM. The combined organic layer was dried over Na₂SO₄ and evaporated under vacuum. The crude was purified by reverse phase column chromatography (acetonitrile/water, 70:30) to give title compound as pale yellow solid (0.13 g, 74.8%). LCMS: 964.4 [M+H]⁺.

### Synthesis of (4S)-4-(2-(2-bromoacetamido)acetamido)-5-((1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[2.5]octan-6-yl)amino)-5-oxopentanoic acid (INX-A9)

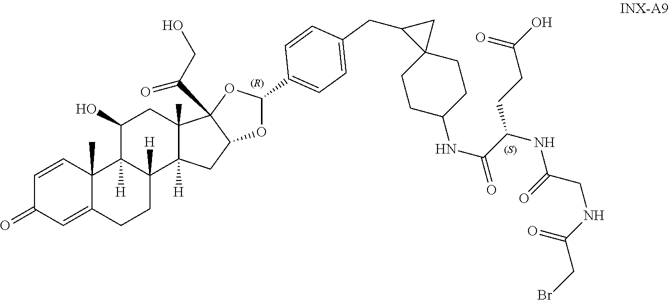

### Procedure

A 10 mL single-necked round bottom flash was charged with tert-butyl (4S)-4-(2-(2-bromoacetamido)acetamido)-5-((1-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[2.5]octan-6-yl)amino)-5-oxopentanoate (INX-A9-3) (0.13 g, 0.13 mmol) and DCM (2 mL). To this solution, TFA (0.38 g, 3.3 mmol) was added and stirred for 2 h at room temperature. After completion of reaction as indicated by TLC, reaction mixture was evaporated under vacuum (0.11 g, Crude). The crude was purified by The crude was purified by prep-HPLC (Column: SUNFIRE Prep C18 OBD, 19×250 mm, 5 μm, Mobile phase: A=0.05% TFA in Water, B=acetonitrile; A:B, 60:40), retention time 14.4 min) to give title compound as off white solid (0.007 g, 5.72%). LCMS: 908.40 [M+H]⁺; ¹H NMR (400 MHZ, MeOD): 0 7.49-7.27 (m, 5H), 6.28 (d, J=10 Hz, 1H), 6.06 (s, 1H), 5.48 (s, Acetal-H, 1H), 5.08 (d, J=5.2 Hz, C16H, 1H), 4.70-3.70 (m, 9H), 3.00-0.90 (m, 26H), 1.50 (s, 3H), 1.10 (s, 3H), 0.54-0.51 (m, 1H), 0.19-0.17 (m, 1H).

### Synthesis of (S)-4-(2-(2-bromoacetamido)acetamido)-5-((4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[2.2.2]octan-1-yl)amino)-5-oxopentanoic acid (INX-Z)

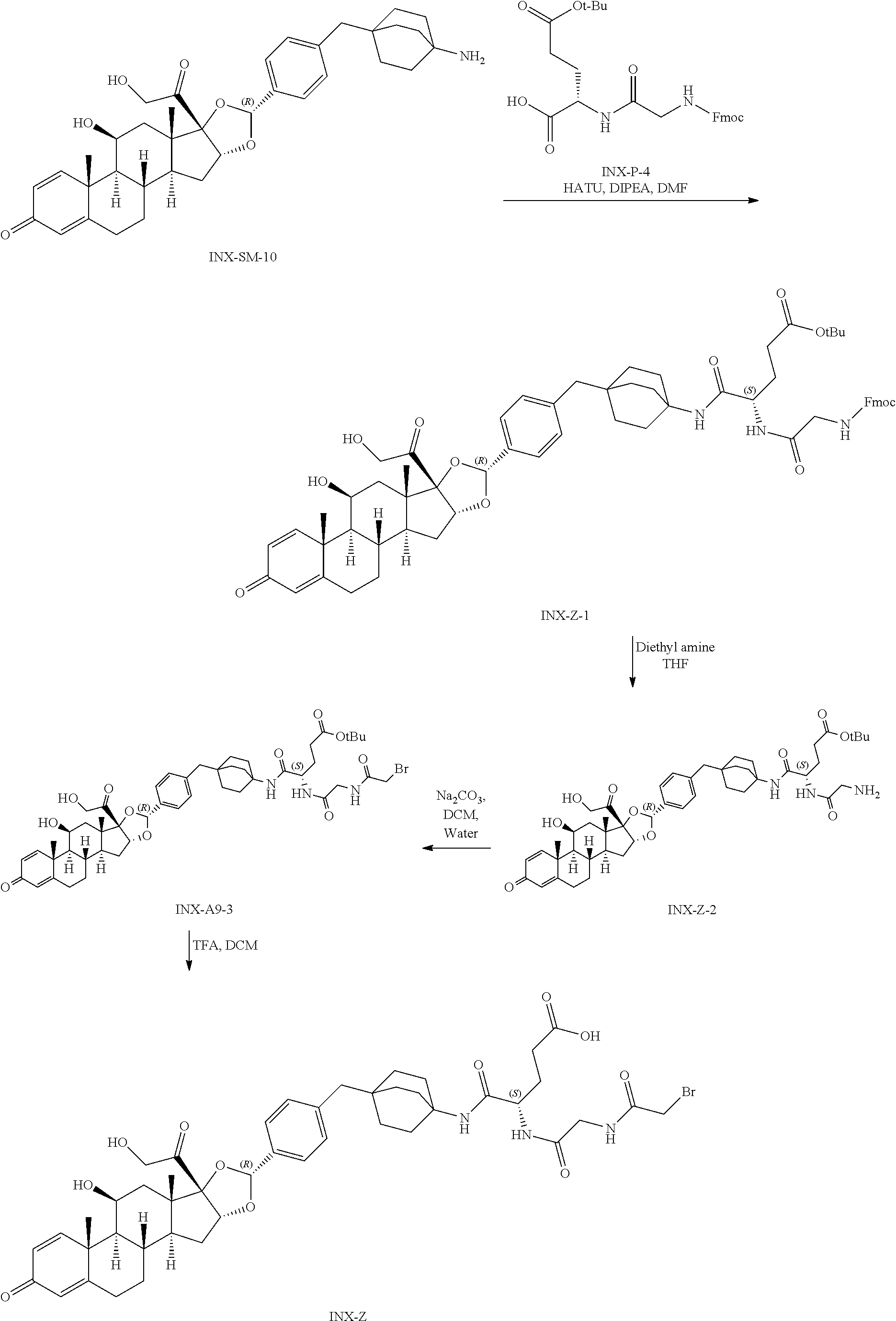

### Synthesis of tert-butyl(S)-4-(2-((((9H-fluoren-9-yl)methoxy)carbonyl)amino)acetamido)-5-((4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[2.2.2]octan-1-yl)amino)-5-oxopentanoate (INX-Z-1)

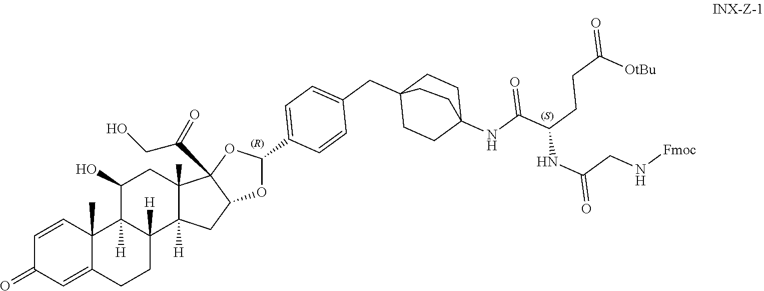

### Procedure

A 10 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) (0.20 g, 0.41 mmol), and (6aR,6bS,7S,8aS,8bS,11aR,12aS,12bS)-10-(4-((4-aminobicyclo[2.2.2]octan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-10) (0.28 g, 0.46 mmol) was taken in DMF (2 mL). To this solution DIPEA (0.21 g, 1.6 mmol) and HATU (0.44 g, 1.1 mmol) were added and stirred for 1 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water: 50:50) to give title compound as pale yellow solid (0.1 g, 20.16%). LCMS 1066.9 [M+H]⁺.

### Synthesis of tert-butyl(S)-4-(2-aminoacetamido)-5-((4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[2.2.2]octan-1-yl)amino)-5-oxopentanoate (INX-Z-2)

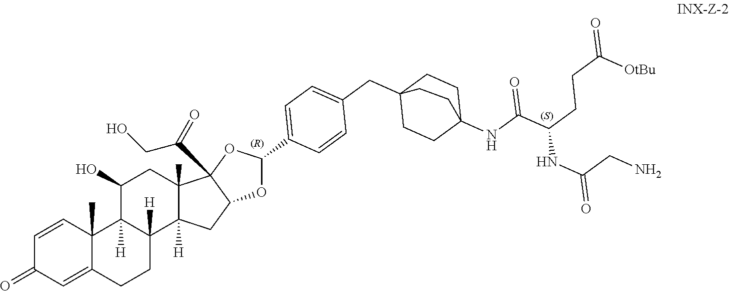

### Procedure

A 10 mL single-necked round bottom flash was charged with tert-butyl (4S)-4-(2-((((9H-fluoren-9-yl)methoxy)carbonyl)amino)acetamido)-5-((4-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[2.2.2]octan-1-yl)amino)-5-oxopentanoate (INX-Z-1) (0.15 g, 0.14 mmol) and THF (5 mL). To this solution, diethyl amine (0.10 g, 1.4 mmol) was added and stirred for 3 h at room temperature. After completion of reaction as indicated by TLC, reaction mixture was evaporated under vacuum and triturated with diethyl ether and pentane to give title compound as yellow solid (0.08 g, 67.38%). LCMS: 844.6 [M+H]⁺.

### Synthesis of tert-butyl(S)-4-(2-(2-bromoacetamido)acetamido)-5-((4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[2.2.2]octan-1-yl)amino)-5-oxopentanoate (INX-Z-3)

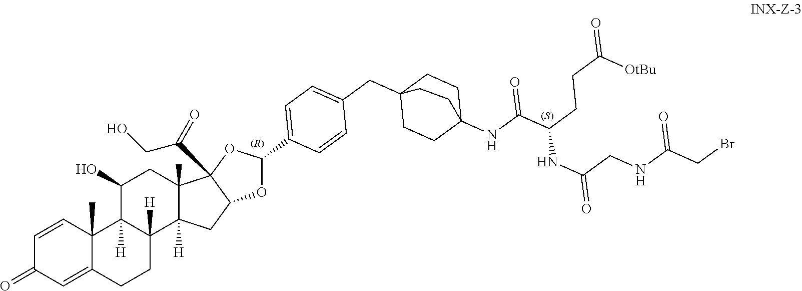

### Procedure

A 10 mL single-necked round bottom flash was charged with tert-butyl (4S)-4-(2-aminoacetamido)-5-((4-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[2.2.2]octan-1-yl)amino)-5-oxopentanoate (INX-Z-2) (0.08 g, 0.094 mmol) and DCM (2 mL). To this solution, Na₂CO₃ (0.030 g, 0.28 mmol) solution in water (1 mL) and bromoacetyl bromide (0.024 g, 0.12 mmol) were added dropwise at room temperature and stirred the reaction mixture for 1 h. After completion of reaction as indicated by TLC, reaction mixture was quenched with water and extracted with DCM. The combined organic layer was dried over Na₂SO₄ and evaporated under vacuum to give title compound as pale yellow solid (0.08 g, 88.2%). LCMS: calculated for C50H67⁷⁹BrN₃O₁₁ (964.40), found 964.6 [M+H]⁺.

### Synthesis of (S)-4-(2-(2-bromoacetamido)acetamido)-5-((4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[2.2.2]octan-1-yl)amino)-5-oxopentanoic acid (INX-Z)

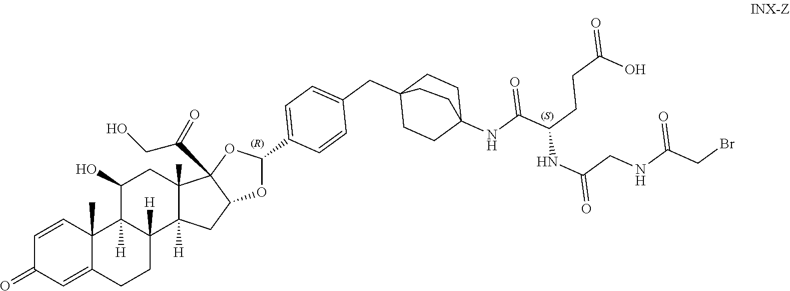

A 10 mL single-necked round bottom flash was charged with tert-butyl (4S)-4-(2-(2-bromoacetamido)acetamido)-5-((4-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[2.2.2]octan-1-yl)amino)-5-oxopentanoate (INX-Z-3) (0.08 g, 0.08 mmol) and DCM (2 mL). To this solution, TFA (0.23 g, 2.10 mmol) was added and stirred the reaction mixture for 2 h at room temperature. After completion of reaction as indicated by TLC, reaction mixture was evaporated under vacuum. The crude was purified by prep HPLC (column YMC-Actus Triart Prep C18-S, 250×20 mm S-5 μm, 12 nm, Mobile phase: A=0.1% FA in water, B=ACN: MEOH (50:50), A:B=35:65, retention time 20.17 min) to give title compound as off white solid (0.005 g, 6.64%) LCMS: calculated for C₄₆H₅₉⁷⁹BrN₃O₁₁ (908.33), found 908.4 [M+H]⁺; ¹H NMR (400 MHZ, MeOD) Key proton assignment): ¹H NMR (400 MHZ, MeOD): 0 7.46 (d, J=10.4 Hz, 1H), 7.34 (d, J=7.6 Hz, 2H), 7.09 (d, J=7.6 Hz, 2H), 6.26 (d, J=9.6 Hz, 1H), 6.04 (s, 1H), 5.44 (s, Acetal-H, 1H), 5.05 (d, J=5.2 Hz, C16H, 1H), 5.00-4.10 (m, 9H), 2.70-1.60 (m, 20H), 1.51 (s, 3H), 1.50-1.40 (m, 6H), 1.25-1.05 (m, 2H), 1.00 (s, 3H).

### Synthesis of (6aR,6bS,7S,8aS,8bS,11aR,12aS,12bS)-10-(4-((4-amino-2-oxabicyclo[2.2.2]octan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-36)

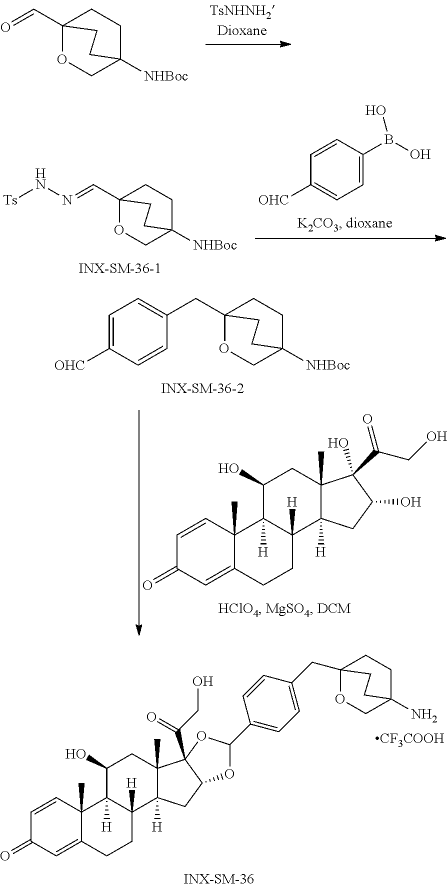

### Synthesis of tert-butyl (E)-(1-((2-tosylhydrazono)methyl)-2-oxabicyclo[2.2.2]octan-4-yl)carbamate (INX-SM-36-1)

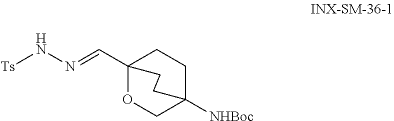 [SMILES: Cc1ccc(S(=O)(=O)N/N=C/C23CCC(NC(=O)OC(C)(C)C)(CC2)CO3)cc1]

### Procedure

A 50 mL single-necked round bottom flash was charged with tert-butyl (1-formyl-2-oxabicyclo[2.2.2]octan-4-yl)carbamate (0.2 g, 0.78 mmol) and EtOH (5 mL) under nitrogen. To this solution, p-toluenesulfonhydrazide (0.218 g, 1.17 mmol) and catalytic amount of AcOH (0.1 mL) were added and stirred the reaction mixture for 0.5h at room temperature. After completion of reaction as indicated by TLC, reaction mixture was poured into water and the resulted solid was filtered and dried under vacuum to give title compound as white solid (0.30 g, 90.42%). LCMS: 424.23 [M+H]⁺

### Synthesis of tert-butyl (1-(4-formylbenzyl)-2-oxabicyclo[2.2.2]octan-4-yl)carbamate (INX-SM-36-2)

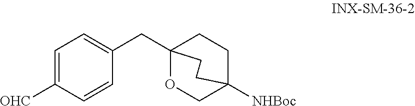 [SMILES: CC(C)(C)OC(=O)NC12CCC(Cc3ccc(C=O)cc3)(CC1)OC2]

### Procedure

A 35 mL glass vial was charged with tert-butyl (E)-(1-((2-tosylhydrazono)methyl)-2-oxabicyclo[2.2.2]octan-4-yl)carbamate (INX-SM-36-1) (0.1 g, 0.236 mmol) and dioxane (3 mL) under nitrogen. To this solution, (4-formylphenyl)boronic acid (0.053 g, 0.354 mmol) and K2CO3 (0.048 g, 0.354 mmol) were added and stirred at 100° C. for 3 h. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane, 30:70) to give title compound as yellow sticky solid (0.030 g, 36.78%). LCMS: 346.2 [M+H]⁺

### Synthesis of (6aR,6bS,7S,8aS,8bS,11aR,12aS,12bS)-10-(4-((4-amino-2-oxabicyclo[2.2.2]octan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-36)

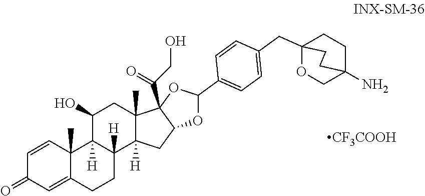

A 50 mL single-necked round bottom round bottom flash was charged with tert-butyl (1-(4-formylbenzyl)-2-oxabicyclo[2.2.2]octan-4-yl)carbamate (INX-SM-36-2) (0.025 g, 0.072 mmol), (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (16-alpha-hydroxyprednisolone) (0.027 g, 0.072 mmol) and DCM (2 mL). To this solution, MgSO₄ (0.043 g, 0.3615 mmol) and HClO₄ (0.036 g, 0.3615 mmol) were added and stirred for 2 h at room temperature. After completion of reaction as indicated by TLC, reaction mixture was quenched with sat. NaHCO₃ solution and concentrated under vacuum. The crude was triturated with cold water and participated solid was filtered and dried under vacuum. The crude was purified by prep-HPLC (Column: YMC-Actus Triart Prep C18-S, 250×20 mm S-5 μm, 12 nm, Mobile phase: A=0.05% TFA in water, B=acetonitrile; A:B=75:25, retention time 12.52 min) to give title compound as white solid. (0.008 g, 15.54%). LCMS: 604.4 [M+H]⁺; ¹H NMR (400 MHZ, MeOD): δ: 7.47 (d, J=10.0 Hz, 1H), 7.37 (d, J=8.0 Hz, 2H), 7.22 (d, J=8.0 Hz, 2H), 6.27 (dd, J=10.0 & 1.6 Hz 1H), 6.04 (s, 1H), 5.47 (s, Acetal-H, 1H), 5.08 (d, J=4.8 Hz, C16H, 1H), 4.65 (d, J=19.6 Hz, 1H), 4.45-4.40 (m 1H), 4.33 (d, J=19.6 Hz, 1H), 3.30 (s, 2H), 2.73 (s, 2H), 2.70-2.69 (m, 1H), 2.50-1.60 (m, 16H), 1.52 (s, 3H), 1.20-1.04 (m, 2H), 1.01 (s, 3H).

### Synthesis of ((4S)-4-(2-(2-bromoacetamido)acetamido)-5-((1-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoic acid (INX-A24)

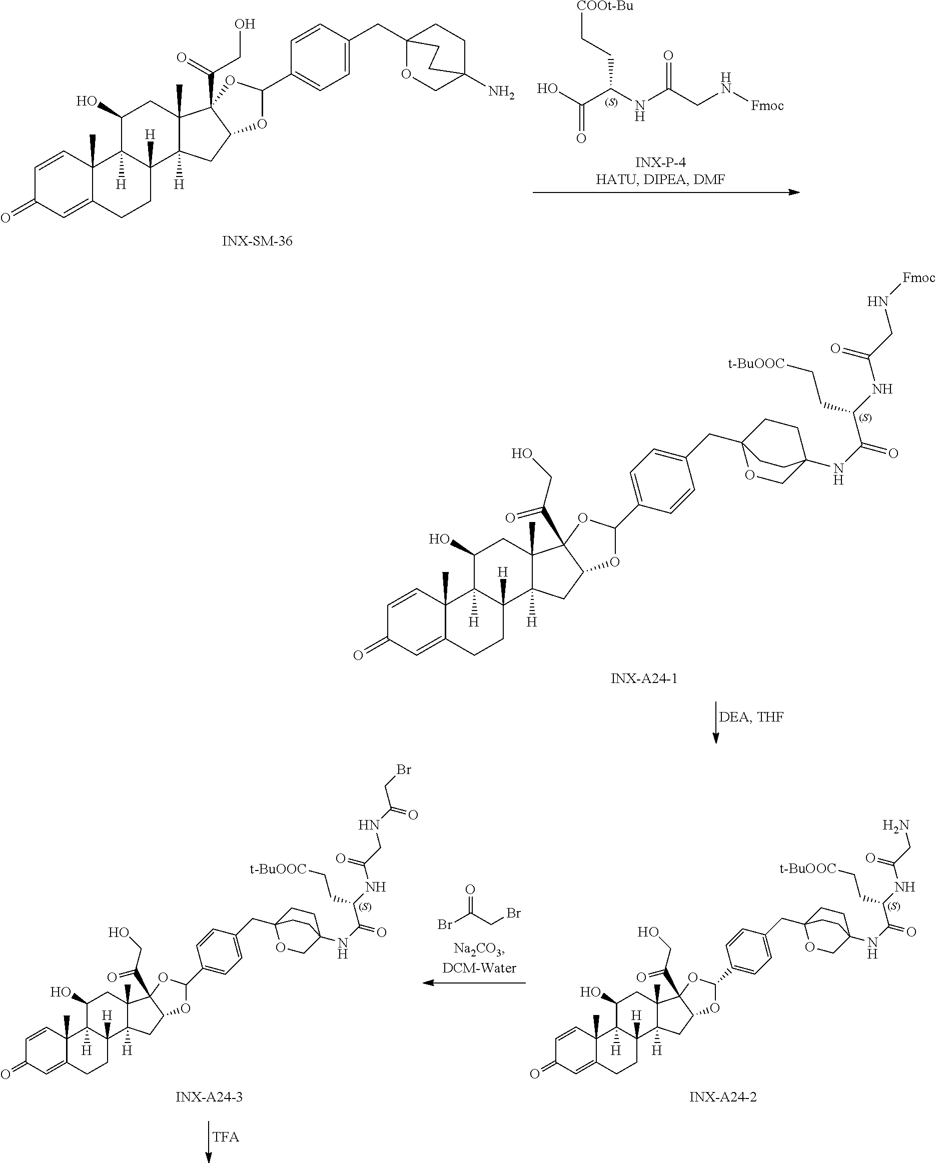

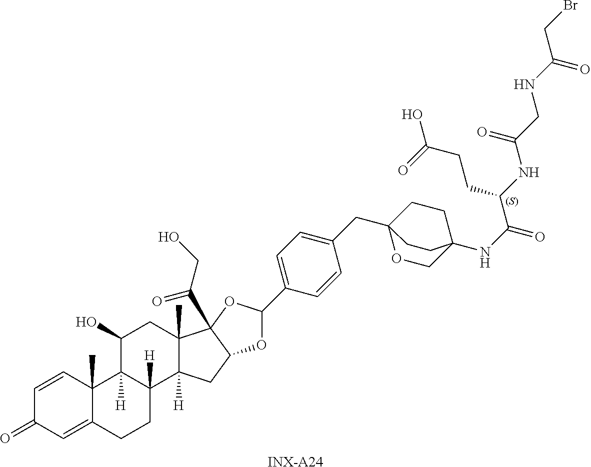

### Synthesis of tert-butyl (4S)-4-(2-((((9H-fluoren-9-yl)methoxy)carbonyl)amino)acetamido)-5-((1-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A24-1)

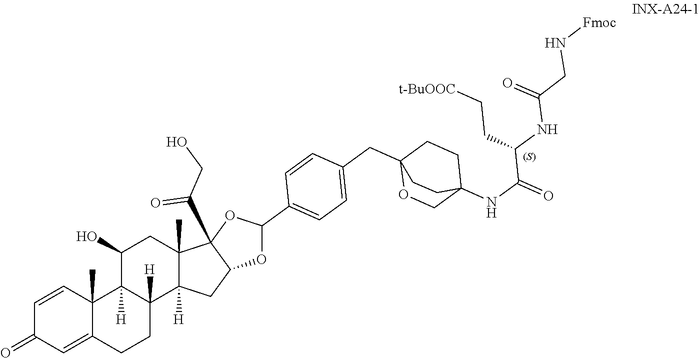

### Procedure

A 50 mL single-necked round bottom flash was charged(S)-2-(2-((((9H-fluoren-9-yl)methoxy)carbonyl)amino)acetamido)-5-(tert-butoxy)-5-oxopentanoic acid (INX-P-4) (0.263 g, 0.546 mmol), HATU (0.207 g, 0.546 mmol) and DMF (3 mL). To this solution, DIPEA (0.141 g, 0.109 mmol) followed by (6aR,6bS,7S,8aS,8bS,11aR,12aS,12bS)-10-(4-((4-amino-2-oxabicyclo[2.2.2]octan-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-36) (0.330 g, 0.546 mmol) were added and stirred for 3 h at room temperature. After completion of reaction as indicated by TLC, reaction mixture was poured into water and extracted with ethyl acetate. The combined organic layer was dried over Na₂SO₄ and evaporated under vacuum. The crude was purified by silica gel column chromatography (MeOH/DCM, 10:90) to give title compound as white solid (0.3 g, 51.38%). LCMS: 1068.7 [M+H]⁺

### Synthesis of tert-butyl (4S)-4-(2-aminoacetamido)-5-((1-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A24-2)

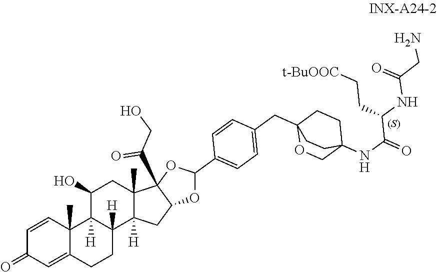

### Procedure

A 25 mL single-necked round bottom flash was charged with tert-butyl (4S)-4-(2-((((9H-fluoren-9-yl)methoxy)carbonyl)amino)acetamido)-5-((1-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A 24-1) (0.5 g, 0.468 mmol) and THF (5 mL). To this solution, diethylamine (0.342 g, 4.68 mmol) was added and stirred for 2.5h at room temperature. After completion of reaction as indicated by TLC, reaction mixture was concentrated under vacuum. The crude was purified by trituration with DCM and hexane to give title compound as white solid (0.35 g, 88.38%).

LCMS: 846.5 [M+H]⁺.

### Synthesis tert-butyl (4S)-4-(2-(2-bromoacetamido)acetamido)-5-((1-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A24-3)

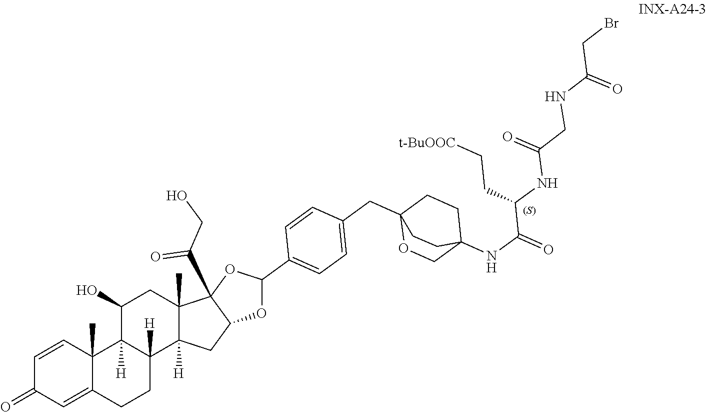

### Procedure

A 25 mL single-necked round bottom flash was charged with tert-butyl (4S)-4-(2-aminoacetamido)-5-((1-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoatev (INX-A24-2) (0.365 g, 0.431 mmol) and DCM (5 mL). To this solution, Na₂CO₃ (0.091 g, 0.862 mmol) dissolved in water (0.5 mL) followed by bromoacetyl bromide (0.087 g, 0.431 mmol) were added and stirred for 0.5h at room temperature. After completion of reaction as indicated by TLC, reaction mixture was quenched with water and extracted with DCM. The combined organic layer was dried over Na₂SO₄ and evaporated under vacuum to give title compound as off white solid (0.22 g, 52.8%). LCMS: 966.4 [M+H]⁺

### Synthesis of ((4S)-4-(2-(2-bromoacetamido)acetamido)-5-((1-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoic acid (INX-A24)

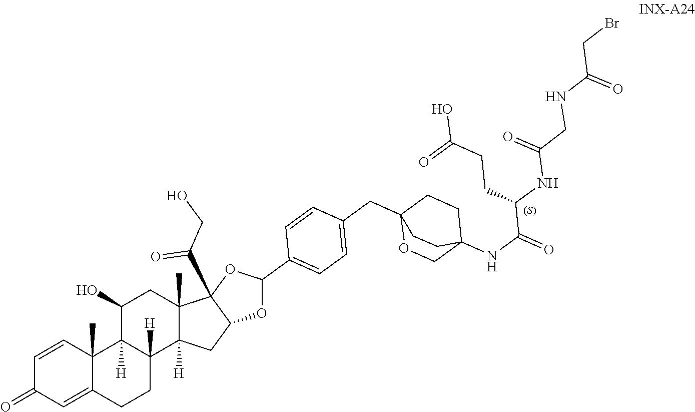

### Procedure

A 25 mL single-necked round bottom flash was charged with tert-butyl (4S)-4-(2-(2-bromoacetamido)acetamido)-5-((1-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A24-3) (0.2 g, 0.206 mmol) and DCM (5 mL). To this solution, TFA (0.5 mL) was added and stirred for 2 h at room temperature. After completion of reaction as indicated by TLC, reaction mixture was evaporated under vacuum. The crude was purified by prep-HPLC (Column: X-bridge Prep, C18, OBD (250×19) mm, 5 μm, Mobile phase: A=0.05% TFA in water, B=Acetonitrile: MeOH: IPA (65:25:10); A:B=70:30, retention time 15.88 min) to give title compound as off white solid (0.025 g, 13.32%). LCMS: calculated for C₄₅H₅₇⁷⁹BrN₃O₁₂ (910.31), found 910.6 [M+H]⁺; ¹H NMR (400 MHZ, MeOD): δ: 7.47 (d, J=10.0 Hz, 1H), 7.37 (d, J=8.0 Hz, 2H), 7.20 (d, J=8.0 Hz, 2H), 6.27 (dd, J=10.0, 1H), 6.04 (s, 1H), 5.46 (s, Acetal-H, 1H), 5.08 (d, J=5.2 Hz, C16H, 1H), 4.64 (d, J=19.6 Hz, 1H), 4.45-4.44 (m 1H), 4.34 (d, J=19.6 Hz, 1H), 4.27-4.22 (m, 1H), 3.98-3.89 (m, 6H), 2.70 (s, 2H), 2.50-1.55 (m, 21H), 1.52 (s, 3H), 1.20-1.04 (m, 2H), 1.01 (s, 3H).

### Synthesis of (4S)-4-(2-(2-bromoacetamido)acetamido)-5-((1-(4-((6aR,6bS,7S,8aS,8bS,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoic acid (INX-A25)

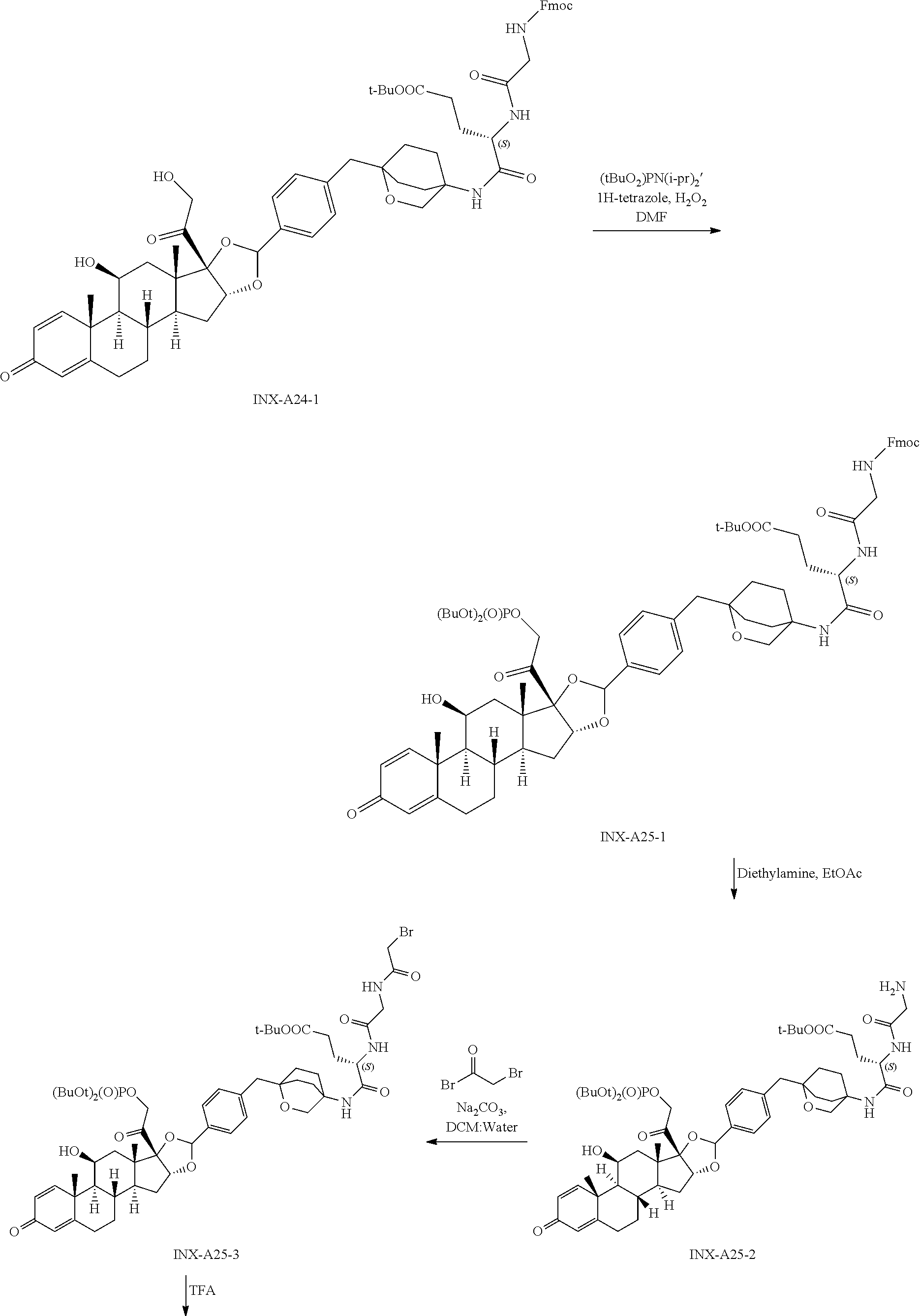

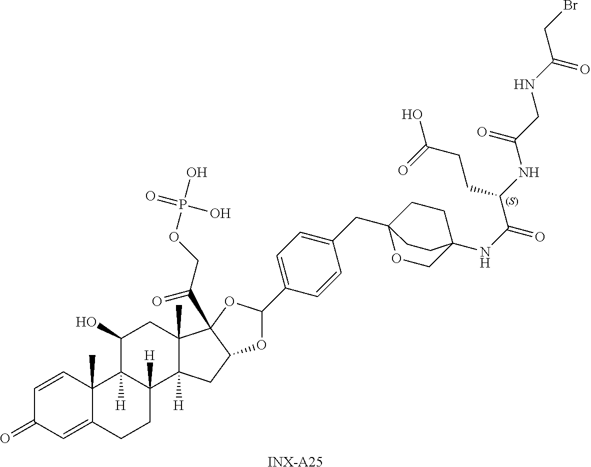

### Synthesis of tert-butyl (4S)-4-(2-((((9H-fluoren-9-yl)methoxy)carbonyl)amino)acetamido)-5-((1-(4-((6aR,6bS,7S,8aS,8bS,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A25-1)

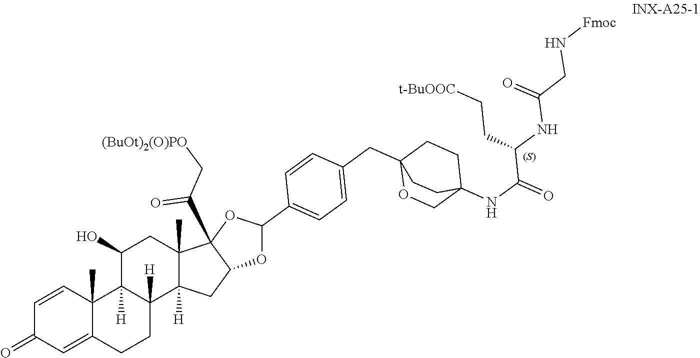

### Procedure

A 35 mL glass vial was charged with tert-butyl (4S)-4-(2-((((9H-fluoren-9-yl)methoxy)carbonyl)amino)acetamido)-5-((1-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A24-1) (0.300 g, 0.280 mmol) and DMF (0.6 mL). To this solution, 1H-tetrazole (0.196 g, 2.800 mmol) and (tBuO)₂PN(iPr)₂ (1.86 g, 6.741 mmol) were added and stirred for 24 h at room temperature. The reaction mixture was cooled to 0° C. and hydrogen peroxide (3 mL) was added into the reaction mixture. Stirred the reaction mixture and then concentrated under vacuum. The crude was purified by reverse phase column chromatography (acetonitrile:water, 70:30) to obtain title compound as white solid (0.140 g, 39.55%). LC/MS: 1260.7 [M+H]⁺

### Synthesis of tert-butyl (4S)-4-(2-aminoacetamido)-5-((1-(4-((6aR,6bS,7S,8aS,8bS,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A25-2)

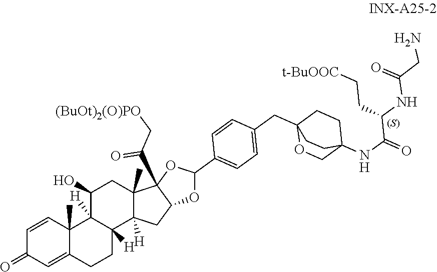

### Procedure

A 35 mL glass vial was charged with tert-butyl (4S)-4-(2-((((9H-fluoren-9-yl)methoxy)carbonyl)amino)acetamido)-5-((1-(4-((6aR,6bS,7S,8aS,8bS,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A25-1) (0.140 g, 0.111 mmol) and EtOAc (2 mL). To this solution, diethyl amine (0.081 g, 1.111 mmol) was added and stirred for 2 h at room temperature. After completion of reaction as indicated by TLC, reaction mixture was diluted with EtOAc and washed with H₂O. The organic layer was dried over Na₂SO₄ and concentrated under vacuum. The crude was triturated with hexane to obtain title compound as white solid (0.080 g, 69.37%). LC/MS: 1039.0 [M+H]⁺

### Synthesis of tert-butyl (4S)-4-(2-(2-bromoacetamido)acetamido)-5-((1-(4-((6aR,6bS,7S,8aS,8bS,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A25-3)

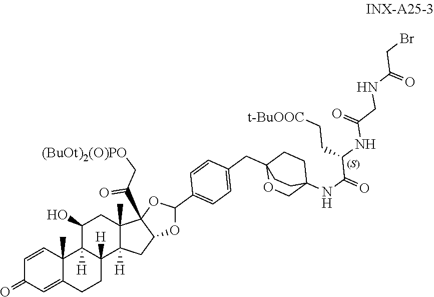

### Procedure

A 35 mL glass vial was charged with tert-butyl (4S)-4-(2-aminoacetamido)-5-((1-(4-((6aR,6bS,7S,8aS,8bS,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A25-2) (0.080 g, 0.077 mmol) and DCM (0.9 mL). To this solution, Na₂CO₃ (0.017 g, 0.154 mmol) dissolved in water (0.1 mL) followed by bromoacetyl bromide (0.016 g, 0.077 mmol) were added at room temperature. The reaction mixture was stirred for 30 min at room temperature. After completion of reaction as indicated by TLC, reaction mixture was quenched with water and extracted with DCM. The combined organic layer was dried over Na₂SO₄ and concentrated under vacuum (0.080 g, 89.57%). LC/MS: 1160.4 [M+H]⁺

### Synthesis of (4S)-4-(2-(2-bromoacetamido)acetamido)-5-((1-(4-((6aR,6bS,7S,8aS,8bS,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoic acid (INX-A25)

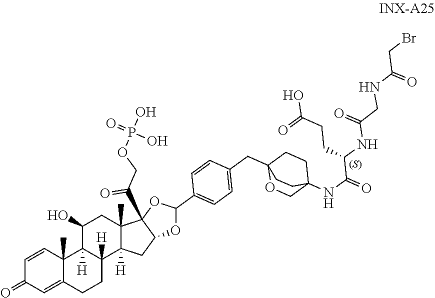

### Procedure

A 25 mL single-necked round bottom flash was charged with tert-butyl (4S)-4-(2-(2-bromoacetamido)acetamido)-5-((1-(4-((6aR,6bS,7S,8aS,8bS,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-oxabicyclo[2.2.2]octan-4-yl)amino)-5-oxopentanoate (INX-A25-3) (0.080 g, 0.069 mmol) and DCM (2 mL). To this solution, TFA (0.2 mL) was added and stirred for 2 h at room temperature. After completion of reaction as indicated by TLC, reaction mixture was evaporated under vacuum. The crude was purified by prep-HPLC (Column: YMC-Actus Triart Prep C18-S, 250×20 mm S-5 μm, 12 nm, Mobile phase: A=0.05% TFA in water, B=Acetonitrile; A:B, 70:30); Retention time 12.88 min) to give title compound as white solid (0.007 g, 10.24%). LCMS: calculated for C₄₅H₅₈⁷⁹BrN₃O₁₅P (990.28), found 990.5 [M+H]⁺; ¹H NMR (400 MHZ, MeOD): δ: 7.51-7.47 (m, 1H), 7.36 (d, J=8.4 Hz, 2H), 7.20 (d, J=8.0 Hz, 2H), 6.27 (dd, J=10.0 & 1.6 Hz, 1H), 6.04 (s, 1H), 5.52 (s, Acetal-H, 1H), 5.07 (d, J=5.2 Hz, C16H, 1H), 4.85-4.80 (m, 1H). 4.50-4.40 (m, 1H), 4.35-4.20 (m, 1H), 4.00-3.80 (m, 6H), 2.70-1.55 (m, 24H), 1.52 (s, 3H), 1.25-1.05 (m, 2H), 1.03 (s, 3H)

### Synthesis of (S)-4-(2-(2-bromoacetamido)acetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoic acid (INX-A17)

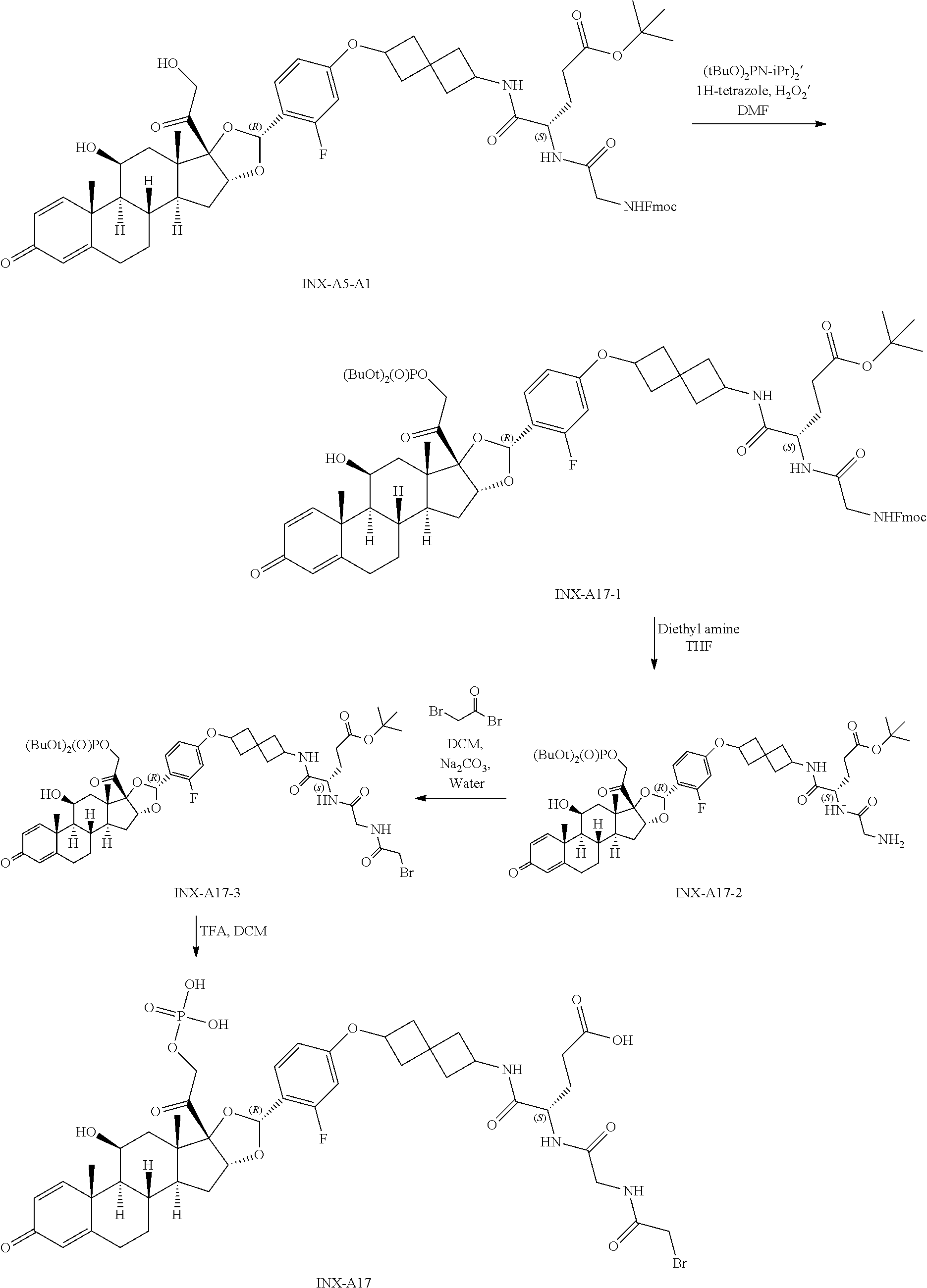

### Synthesis of tert-butyl(S)-4-(2-((((9H-fluoren-9-yl)methoxy)carbonyl)amino)acetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A17-1)

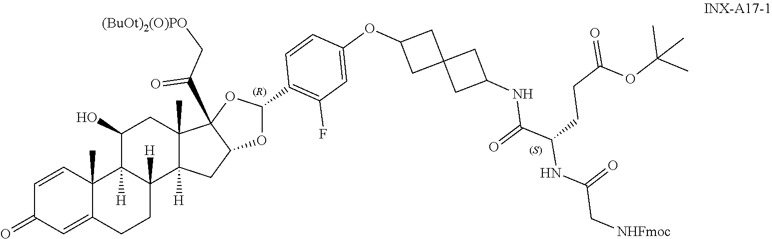

### Procedure

A 35 mL glass vial was charged with (6aR,6bS,7S,8aS,8bS,10R,11aR,12aS,12bS)-10-(4-((6-aminospiro[3.3]heptan-2-yl)oxy)-2-fluorophenyl)-7-hydroxy-8b-(2-hydroxyacetyl)-6a,8a-dimethyl-1,2,6a,6b,7,8,8a,8b,11a,12,12a,12b-dodecahydro-4H naphtho[2′,1′:4,5]indeno[1,2-d][1,3]dioxol-4-one (INX-A5-1) (0.3 g, 0.280 mmol) and DMF (1 mL). To this solution, 1H-tetrazole (0.19 g, 2.80 mmol) and (tBuO)₂PN(iPr)₂ (1.8 g, 6.72 mmol) were added at room temperature and stirred for 16 h. After completion of reaction as indicated by TLC, hydrogen peroxide (3 ml) was added at 0° C. and stirred for 2 h. The crude was subjected to reverse phase column chromatography (acetonitrile:water, 80:20) to give title compound as light yellow solid (0.18 g, 50.84%). LCMS: 1264.5 [M+H]⁺

### Synthesis of tert-butyl(S)-4-(2-aminoacetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A 17-2)

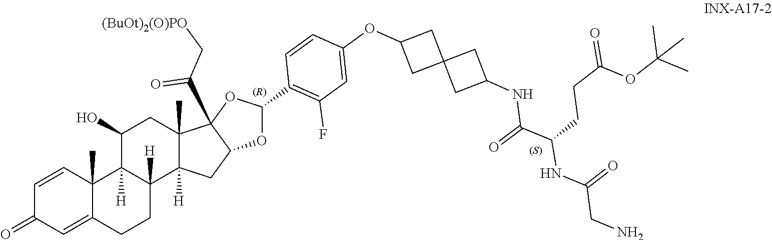

### Procedure

A 35 mL glass vial was charged with tert-butyl(S)-4-(2-((((9H-fluoren-9-yl)methoxy)carbonyl)amino)acetamido)-5-((6-(3-fluoro-4 ((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A17-1) (0.18 g, 0.17 mmol) and THF (1 mL). To this solution, diethyl amine (0.125 g, 1.70 mmol) was added and stirred the reaction mixture for 16 h at room temperature. After completion of reaction as indicated by TLC, reaction mixture was evaporated under vacuum. The crude was triturated with diethyl ether and hexane to give title compound as yellow solid (0.15 g, crude). LCMS: 1042.9 [M+H]⁺

### Synthesis of tert-butyl(S)-4-(2-(2-bromoacetamido)acetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A17-3)

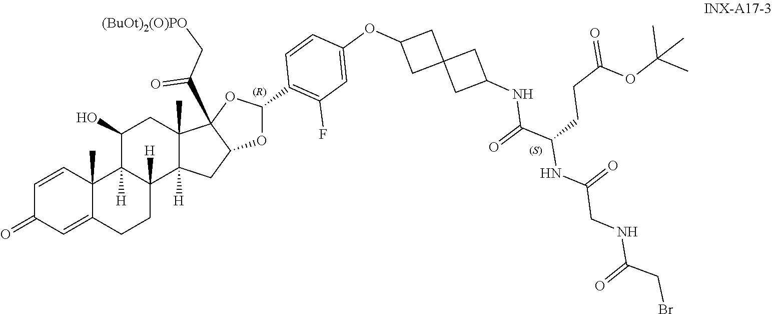

A 10 mL glass vial was charged with tert-butyl(S)-4-(2-aminoacetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A17-2) (0.13 g, 0.14 mmol) and DCM (1 mL). To this solution, Na₂CO₃ (0.030 g, 0.28 mmol) dissolved in water (0.3 mL) followed by bromoacetyl bromide (0.043 g, 0.21 mmol) were added and stirred for 1 h at room temperature. After completion of reaction as indicated by TLC, reaction mixture was quenched with water and extracted with DCM. The combined organic layer was dried over Na₂SO₄ and evaporated under vacuum to give crude title compound as off white solid (0.12 g, 73.69%) LCMS: 1162.6 [M+H]⁺

### Synthesis of (S)-4-(2-(2-bromoacetamido)acetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoic acid (INX-A17)

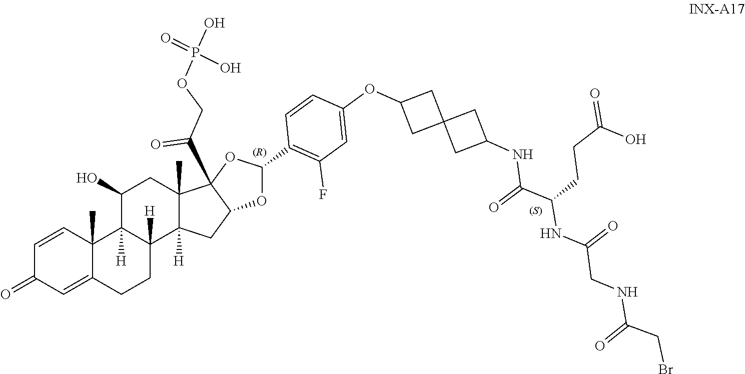

### Procedure

A 10 mL single-necked round bottom flash was charged with tert-butyl(S)-4-(2-(2-bromoacetamido)acetamido)-5-((6-(3-fluoro-4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A17-3) (0.1 g, 0.085 mmol) and DCM (1 mL). To this solution, TFA (0.049 g, 0.42 mmol) was added at room temperature and stirred for 20 min. After completion of reaction as indicated by TLC, reaction mixture was evaporated under vacuum. The crude was purified by prep-HPLC (Column: YMC-Actus Triart Prep C18-S, 250×20 mm S-5 μm, 12 nm, Mobile phase: A=0.05% TFA in water, B=acetonitrile; A:B, 67:33, retention time 10.33 min) to give title compound as white solid (0.010 g, 11.71%). LCMS: 994.3 [M+H]⁺; ¹H NMR (400 MHZ, MeOD): δ: 7.49-7.42 (m, 2H), 6.68 (dd, J=8.8 & 2 Hz, 1H), 6.58-6.55 (m, 1H), 6.28-6.25 (dd, J=10 & 1.6 Hz, 1H), 6.04 (s, 1H), 5.69 (s, 1H), 5.03 (d, J=5.2 Hz, 1H), 4.95-4.74 (m, 2H), 4.63-4.60 (m, 1H), 4.45-4.44 (m, 1H), 4.35-4.31 (m, 1H), 4.24-4.18 (m, 1H), 3.95-3.90 (m, 4H), 2.69-1.72 (m, 21H), 1.52 (s, 3H), 1.31-1.05 (m, 2H), 1.02 (m, 3H).

### Synthesis of (S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)-3-fluorophenoxy) spiro[3.3]heptan-2-yl)amino)-5-oxopentanoic acid (INX-A18)

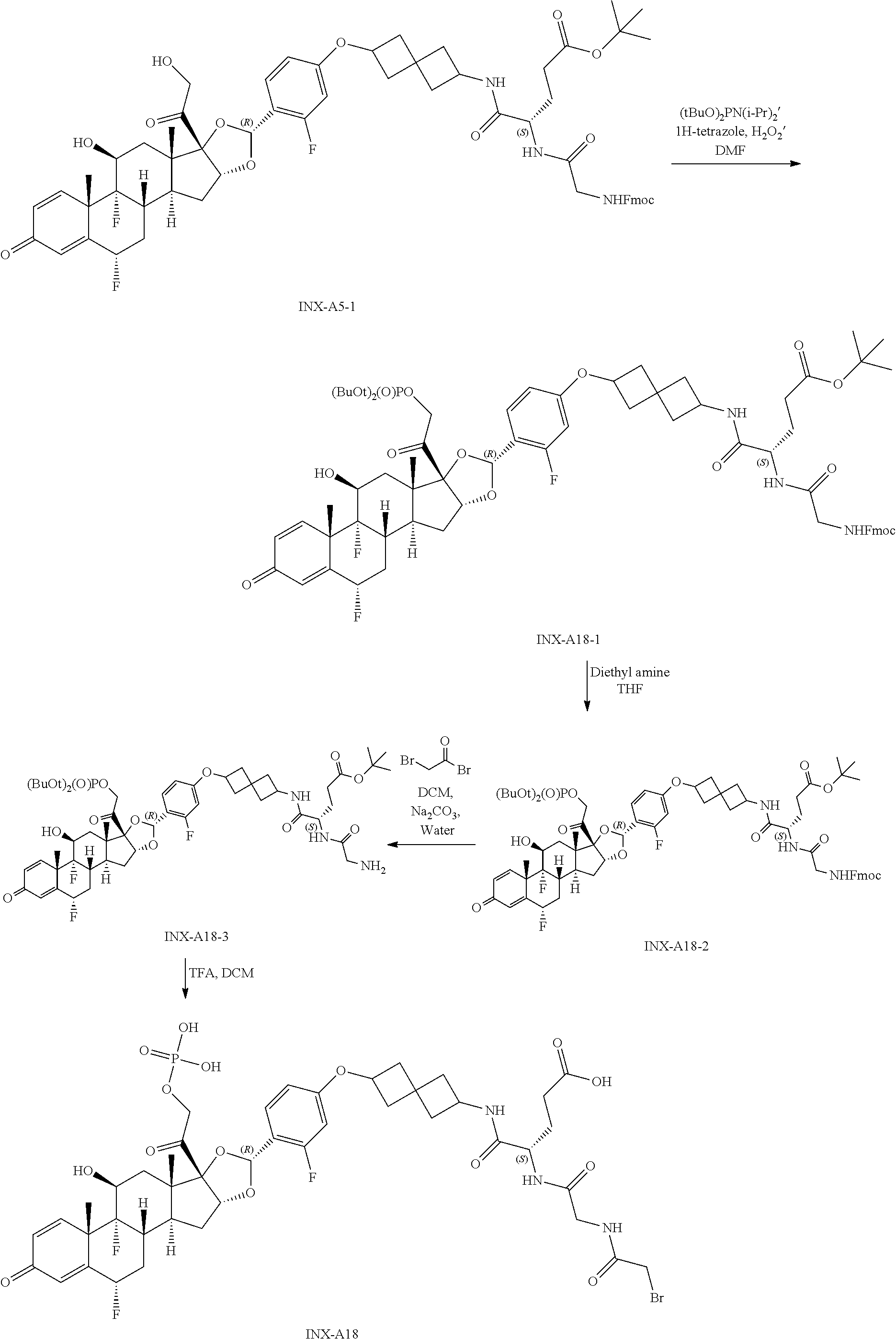

### Synthesis of tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-3-fluorophenoxy) spiro[3.3]heptan-2-yl)amino)-5-oxopentanoate (INX-A18-1)

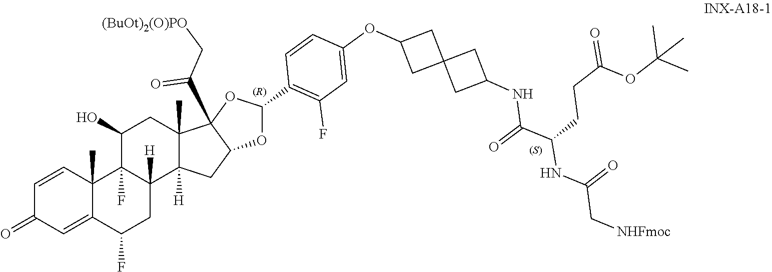

A 35 mL glass vial was charged with tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-3-fluorophenoxy) spiro[3.3]heptan-2-yl)amino)-5-oxopentanoate (INX-A5-1) (0.3 g, 0.270 mmol) and DMF (1 mL). To this solution, 1H-tetrazole (0.189 g, 2.70 mmol) and (tBuO)₂PNEt₂ (1.7 g, 6.48 mmol) were added and stirred for 16 h. The reaction mixture was cooled to 0° C. and hydrogen peroxide (1 mL) was added. The reaction mixture was subjected to reverse phase column chromatography (acetonitrile: water, 80:20) to give title compound as pale yellow solid (0.15 g, 42.65%). LCMS: 1300.51 [M+H]⁺.

### Synthesis of tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-3-fluorophenoxy) spiro[3.3]heptan-2-yl)amino)-5-oxopentanoate (INX-A18-2)

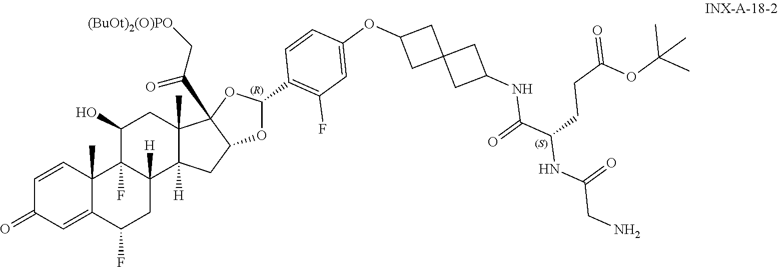

### Procedure

A 35 mL glass vial was charged with tert-butyl(S)-4-(2-(2-bromoacetamido)acetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-3-fluorophenoxy) spiro[3.3]heptan-2-yl)amino)-5-oxopentanoate (INX-A18-1) (0.12 g, 0.09 mmol) and THF (1 mL). To this solution, diethyl amine (0.067 g, 0.92 mmol) was added and stirred for 16 h at room temperature. After completion of reaction as indicated by TLC, reaction mixture was concentrated the solid was triturated with diethyl ether and hexane to give title compound as yellow solid (0.09 g, 92.74%). LCMS: 1078.8 [M+H]⁺.

### Synthesis of tert-butyl(S)-4-(2-aminoacetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-3-fluorophenoxy) spiro[3.3]heptan-2-yl)amino)-5-oxopentanoate (INX-A18-3)

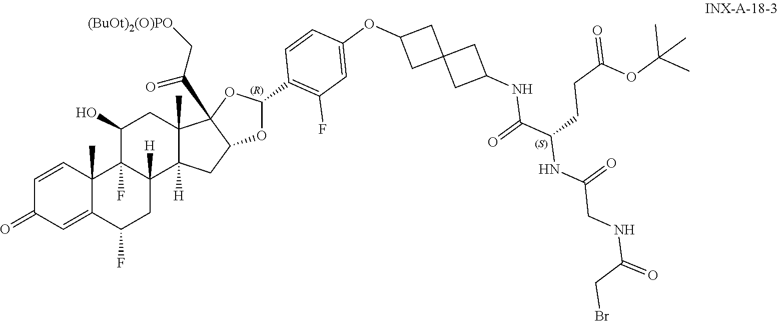

### Procedure

A 10 mL glass vial was charged with tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS}-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-3-fluorophenoxy) spiro[3.3]heptan-2-yl)amino)-5-oxopentanoate (INX-A18-2) (0.090 g, 0.083 mmol) and DCM (1 mL). To this solution, Na₂CO₃ (0.017 g, 0.166 mmol) dissolved in water (0.3 mL) followed by bromoacetyl bromide (0.025 g, 0.12 mmol) were added and stirred for 1 h at room temperature. After completion of reaction as indicated by TLC, reaction mixture was quenched with water and extracted with DCM. The combined organic layer was dried over Na₂SO₄ and evaporated under vacuum to give crude product comprising title compound as off white solid (0.090 g, 90.42%) LCMS: 1199.6 [M+H]⁺.

### Synthesis of (S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)-3-fluorophenoxy) spiro[3.3]heptan-2-yl)amino)-5-oxopentanoic acid (INX-A18)

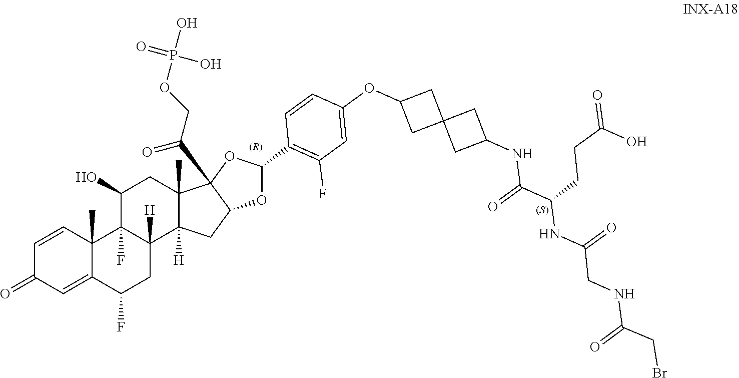

A 10 mL single-necked round bottom flash was charged with tert-butyl(S)-4-(2-aminoacetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-3-fluorophenoxy) spiro[3.3]heptan-2-yl)amino)-5-oxopentanoate (INX-A18-3) (0.090 g, 0.075 mmol) and DCM (1 mL). To this solution, TFA (0.042 g, 0.37 mmol) was added and stirred for 20 min at room temperature. After completion of reaction as indicated by TLC, reaction mixture was evaporated under vacuum. The crude was purified by prep-HPLC (Column: YMC-Actus Triart Prep C18-S, 250×20 mm S-5 μm, 12 nm, Mobile phase: A=0.05% TFA in water, B=acetonitrile; A:B, 67:33, retention time 11.32 min) to give title compound as white solid (0.010 g, 12.93%). LCMS: 1031.2 [M+H]⁺. ¹H NMR (400 MHZ, MeOD): 0 7.44 (t, J=8.4 Hz, 1H), 7.36 (d, J=10 Hz, 1H), 6.68 (m, J=8.4 & 2 Hz, 1H), 6.60-6.55 (m, 1H), 6.37-6.33 (m, 2H), 5.73 (s, Acetal-H, 1H), 5.66-5.49 (m, CH—F, 1H), 5.05 (d, J=4.4 Hz, C16H, 1H), 4.80-4.74 (m, 2H), 4.63-4.60 (m, 1H), 4.35-4.31 (m, 2H), 4.30-4.26 (m, 1H), 3.95-3.90 (m, 4H), 2.68-1.55 (m, 20H), 1.50 (s, 3H), 1.02 (s, 3H).

### Synthesis of (S)-4-(2-(2-bromoacetamido)acetamido)-5-((6-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX-A7)

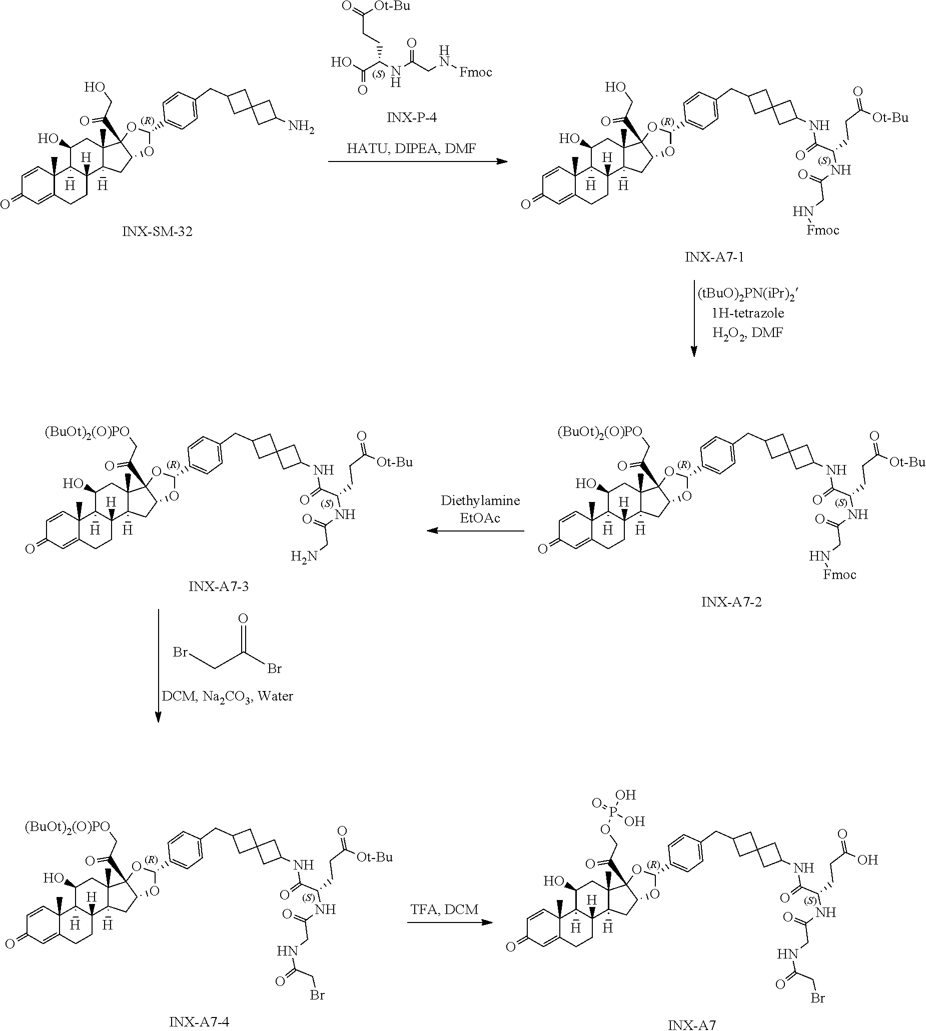

### Synthesis of tert-butyl(S)-4-(2-((((9H-fluoren-9-yl)methoxy)carbonyl)amino)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A7-1)

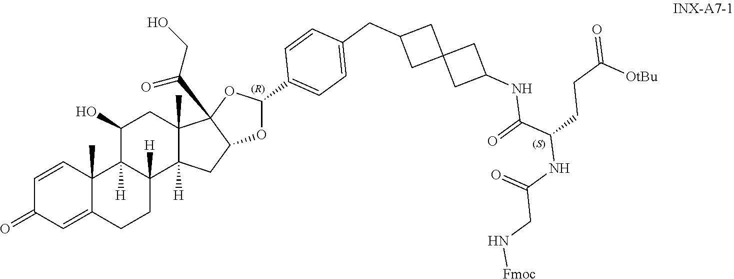

### Procedure

A 35 mL glass vial was charged with(S)-2-(2-((((9H-fluoren-9-yl)methoxy)carbonyl)amino)acetamido)-5-(tert-butoxy)-5-oxopentanoic acid (INX-P-4) (1.14 g, 2.385 mmol), HATU (1.35 g, 3.577 mmol) and DMF (14 mL) under N₂. To this solution, DIPEA (0.8 ml, 4.77 mmol) followed by (6aR,6bS,7S,8aS,8bS,10R,11aR,12aS,12bS)-10-(4-((6-aminospiro[3.3]heptan-2-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-32) (1.4 g, 2.385 mmol) were added at room temperature and stirred for 2 h. After completion of reaction as indicated by TLC, reaction mixture was poured into water and solid was filtered and dried under vacuum. The crude was purified by silica gel column chromatography (MeOH: DCM 5:95) to give title compound as white solid (1.6 g, 63.84%). LCMS: 1052.6 [M+H]⁺.

### Synthesis of tert-butyl(S)-4-(2-((((9H-fluoren-9-yl)methoxy)carbonyl)amino)acetamido)-5-((6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A7-2)

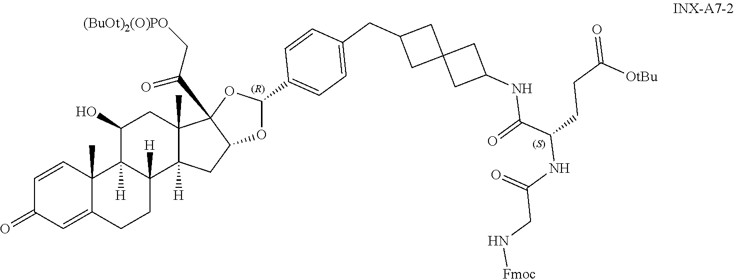

A 35 mL glass vial was charged with tert-butyl(S)-4-(2-((((9H-fluoren-9-yl)methoxy)carbonyl)amino)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate INX-A7-1) (1.6 g, 1.520 mmol) and DMF (2.5 mL). To this solution, 1H-tetrazole (1.06 g, 15.20 mmol) and (tBuO)₂PN(i-Pr)₂ (10.11 g, 36.501 mmol) were added at room temperature and stirred for 24h. Reaction mixture was cooled to ° C. and hydrogen peroxide (16 mL) was added into the reaction mixture and allowed to stir at room temperature for 1 hr. The reaction mixture was subjected to reverse phase column chromatography (acetonitrile:water, 15:85) to give title compound as white solid (1.2 g, 63.42%). LCMS: 1244.6 [M+H]⁺.

### Synthesis of tert-butyl(S)-4-(2-aminoacetamido)-5-((6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A7-3)

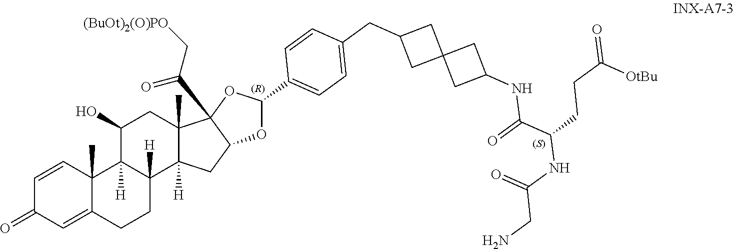

### Procedure

A 50 mL single necked round bottom flask was charged with tert-butyl(S)-4-(2-((((9H-fluoren-9-yl)methoxy)carbonyl)amino)acetamido)-5-((6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A7-2) (1.2 g, 0.964 mmol) and ethyl acetate (12 mL). To this solution, diethyl amine (0.704 g, 9.64 mmol) was added and stirred the reaction mixture for 4 h at room temperature. After completion of reaction as indicated by TLC, reaction mixture was dilute with ethyl acetate and washed with water and organic layer was dried over Na₂SO₄ and concentrated under vacuum. The crude was triturated with hexane to give title compound as white solid (0.650 g, 65.94%). LCMS: 1023.9 [M+H]⁺.

### Synthesis of tert-butyl(S)-4-(2-(2-bromoacetamido)acetamido)-5-((6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A7-4)

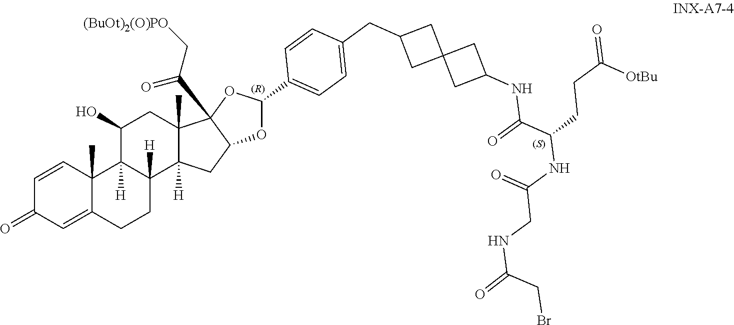

### Procedure

A 25 mL single necked round bottom flask was charged with tert-butyl(S)-4-(2-aminoacetamido)-5-((6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A7-3) (0.650 g, 0.636 mmol) and DCM (9 mL). To this solution, Na₂CO₃ (0.135 g, 1.272 mmol) dissolved in water (1.0 mL) followed by bromoacetyl bromide (0.128 g, 0.636 mmol) were added at room temperature and stirred for 30 min. After completion of reaction as indicated by TLC, reaction mixture was quenched with water and extracted with DCM. The combined organic layer was dried over Na₂SO₄ and concentrated. The crude was purified by reverse phase column chromatography (Acetonitrile: Water, 40:60) to give title compound as yellow solid (0.25 g, 34.79%). LCMS: 1142.6 [M+H]⁺.

### Synthesis of (S)-4-(2-(2-bromoacetamido)acetamido)-5-((6-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX-A7)

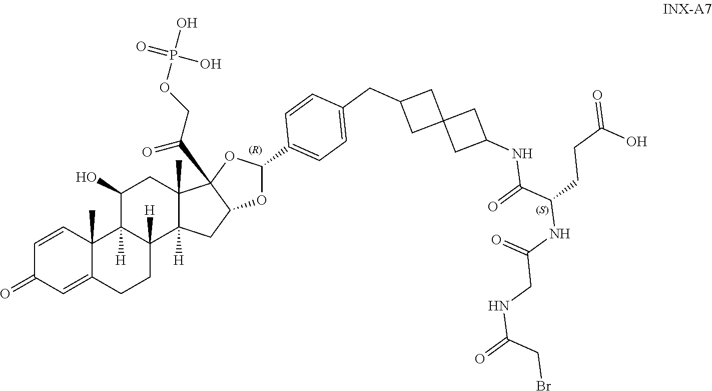

A 25 mL single-necked round bottom flash was charged with tert-butyl(S)-4-(2-(2-bromoacetamido)acetamido)-5-((6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A7-4) (0.250 g, 0.218 mmol) and DCM (5 mL). To this solution, TFA (0.5 mL) was added and stirred the reaction mixture for 2 h at room temperature. After completion of reaction as indicated by TLC, reaction mixture was evaporated under vacuum. The crude was purified by prep-HPLC (Column: YMC-Actus Triart Prep C18-S, 250×20 mm S-5 μm, 12 nm, Mobile phase: A=0.05% TFA in water, B=acetonitrile, A:B=55:45, retention time 19.15 min) to give title compound as white solid (0.026 g, 12.20%). LCMS: 974.3 [M+H]⁺; ¹H NMR (400 MHZ, MeOD): δ: 7.47 (d, J=10.0 Hz, 1H), 7.36 (d, J=8.0 Hz, 2H), 7.16 (d, J=8.0 Hz, 2H), 6.27 (dd, J=10.0 &1.6 Hz, 1H), 6.04 (s, 1H), 5.51 (s, Acetal-H, 1H), 5.06 (d, J=4.8 Hz, C16H, 1H), 5.00-4.70 (m, 2H), 4.46-4.45 (m, 1H), 4.33-4.30 (m, 1H), 4.15-4.09 (m, 1H), 3.94-3.92 (m, 3H), 2.68-1.72 (m, 20H), 1.52 (s, 3H), 1.18-1.05 (m, 2H), 1.03 (s, 3H).

### Synthesis of (S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX-A12)

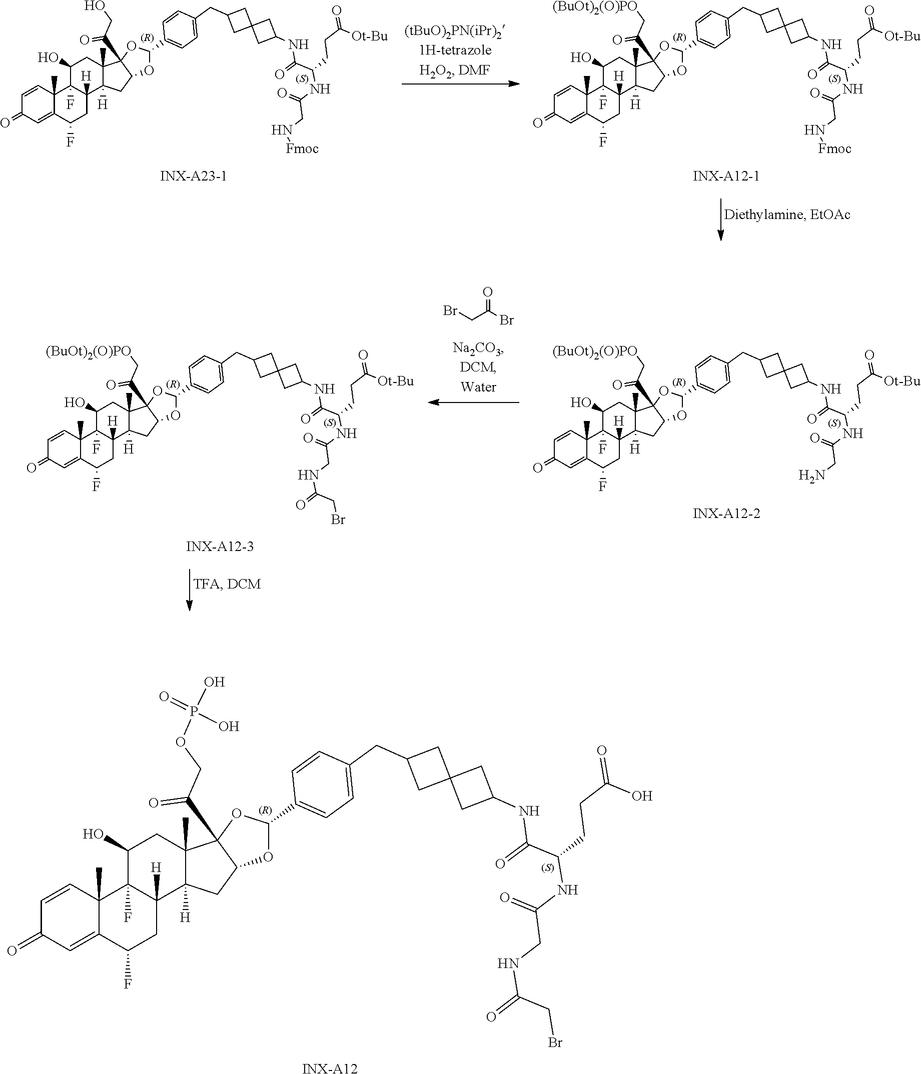

### Synthesis of tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A12-1)

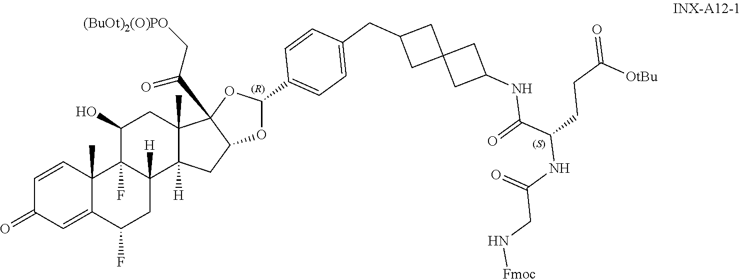

### Procedure

A 35 mL glass vial was charged with tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A23-1) (1.2 g, 1.10 mmol) and DMF (2.0 mL). To this solution, 1H-tetrazole (0.771 g, 11.020 mmol) and (tBuO)₂PN(i-Pr)₂ (7.33 g, 26.46 mmol) were added and stirred for 24 h at room temperature. The reaction mixture was cooled to 0° C. and hydrogen peroxide (12 mL) was added into the reaction mixture and stirred for 1 h. After completion of reaction as indicated by TLC, the reaction mixture was subjected to reverse phase column chromatography (acetonitrile:water, 70:30) to give title compound as white solid (1.1 g, 77.91%). LC/MS: 1280.8 [M+H]⁺

### Synthesis of tert-butyl(S)-4-(2-aminoacetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A12-2)

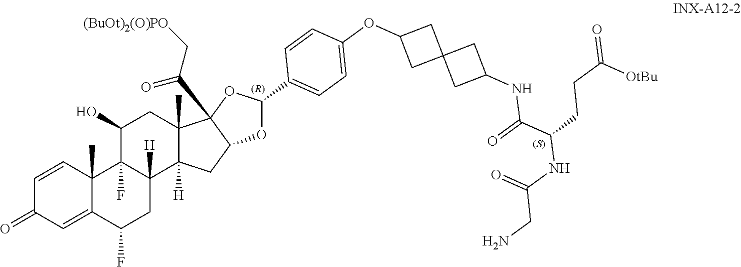

A 35 mL glass vial was charged with tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A12-1) (1.1 g, 0.859 mmol) and EtOAc (11 mL). To this solution, diethyl amine (0.628 g, 8.593 mmol) was added at room temperature and stirred for 2 h at room temperature. After completion of reaction as indicated by TLC, reaction mixture was diluted with EtOAc and washed with water. The organic layer was dried by Na₂SO₄ and concentrated under vacuum. The crude was triturated with hexane and dried under vacuum to obtain title compound as white solid (0.550 g, 60.50%). LC/MS: 1058.8 [M+H]⁺

### Synthesis of tert-butyl(S)-4-(2-(2-bromoacetamido)acetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A12-3)

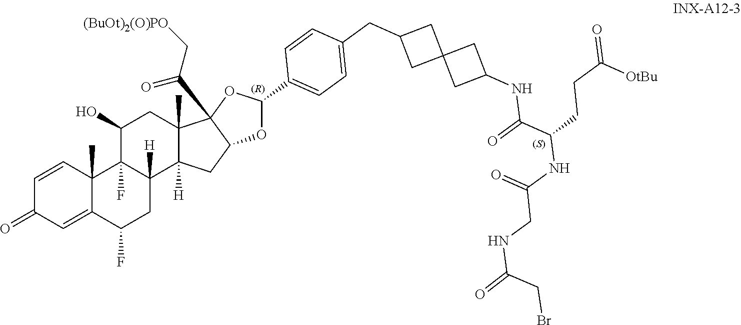

### Procedure

A 35 mL glass vial was charged with tert-butyl(S)-4-(2-aminoacetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A12-2) (0.550 g, 0.519 mmol) and DCM (9 mL). To this solution, Na₂CO₃ (0.110 g, 1.038 mmol) dissolved in water (1.0 mL) and bromoacetyl bromide (0.105 g, 0.0.519 mmol) were added and the reaction mixture was stirred at room temperature for 30 min. After completion of reaction as indicated by TLC, reaction mixture was quenched with water and extracted with DCM. The combined organic layer was dried over Na₂SO₄ and concentrated under reduced pressure to give title compound (0.400 g, 65.27%). LC/MS: 1180.2 [M+H]⁺.

Synthesis of(S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX-A12)

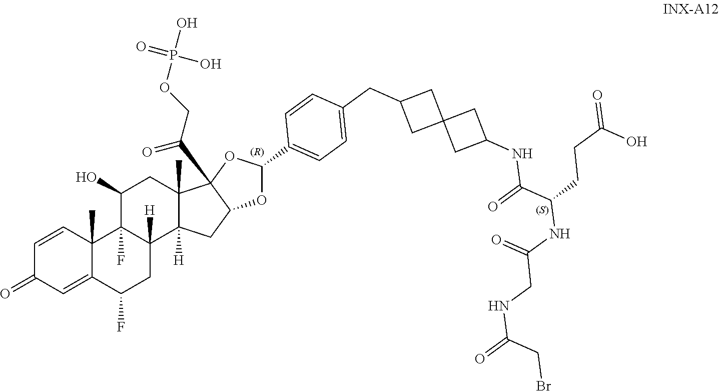

### Procedure

A 25 mL single-necked round bottom flash was charged with tert-butyl(S)-4-(2-(2-bromoacetamido)acetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoate (INX-A12-3) (0.250 g, 0.212 mmol) and DCM (5 mL). To this solution, TFA (0.5 mL) was added and stirred at room temperature for 2 h. After completion of reaction as indicated by TLC, reaction mixture was evaporated under vacuum and the crude was purified by prep-HPLC (Column: SUNFIRE Prep C18 OBD, 19×250 mm, 5 μm, Mobile phase: A=0.05% TFA in water, B=acetonitrile; A:B, 60:40) to give title compound as white solid (0.022 g, 10.27%). LCMS: 1010.4 [M+H]⁺; ¹H NMR (400 MHZ, MeOD): 0 7.37-7.34 (m, 3H), 7.17 (d, J=8.0 Hz, 2H), 6.38-6.35 (m, 2H), 5.70-5.55 (m, CHF, 1H), 5.54 (s, Acetal-H, 1H), 5.07 (d, J=4.8 Hz, C16H, 1H), 5.05-4.80 (m, 3H), 4.35-4.30 (m, 2H), 4.13-4.09 (m, 1H), 3.94-3.89 (m, 3H), 2.68-1.65 (m, 20H), 1.60 (s, 3H), 1.03 (s, 3H)

### Synthesis of (S)-4-(2-(2-bromoacetamido)acetamido)-5-((3-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoic acid (INX-A14)

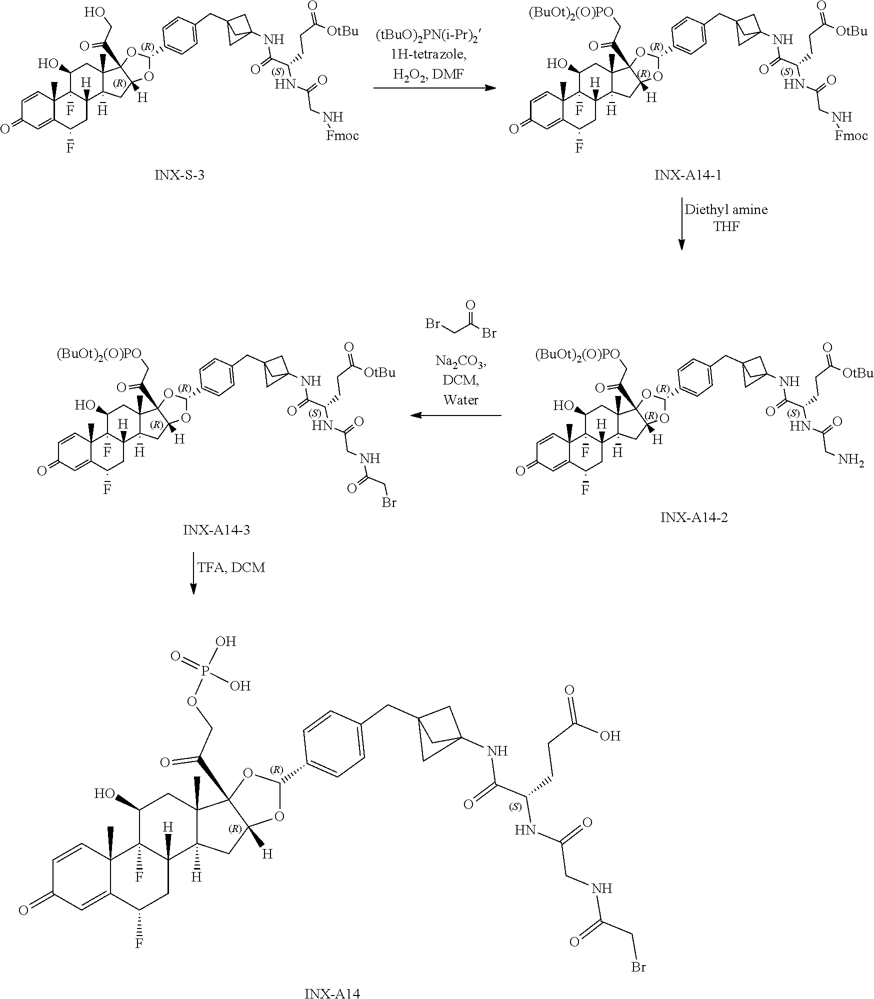

### Synthesis of tert-butyl(S)-4-(2-((((9H-fluoren-9-yl)methoxy)carbonyl)amino)acetamido)-5-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-A14-1)

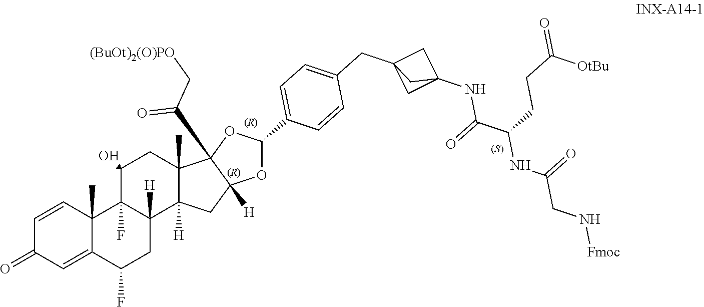

### Procedure

A 35 mL glass vial was charged with tert-butyl(S)-4-(2-((((9H-fluoren-9-yl)methoxy)carbonyl)amino)acetamido)-5-((3-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX—S-3) (0.5 g, 0.471 mmol) and DMF (1 mL). To this solution, 1H-tetrazole (0.33 g, 4.71 mmol) and (tBuO)₂PN(i-Pr)₂ (3.13 g, 11.31 mmol) were added at room temperature and stirred for 24 h. The reaction mixture was cooled at 0° C. and hydrogen peroxide (10V) was added and allowed to stir at room temperature for 1 h. After completion of reaction as indicated by TLC, the reaction mixture was subjected to reverse phase column chromatography (acetonitrile:water, 70:30) to give title compound as light yellow solid (0.28 g, 47.45%). LCMS: 1252.73 [M+H]⁺.

### Synthesis of tert-butyl (S)-4-(2-aminoacetamido)-5-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-A14-2)

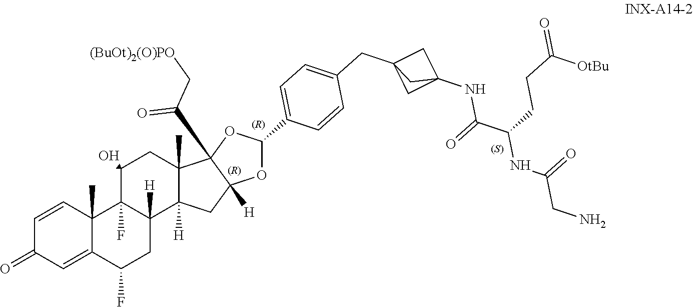

### Procedure

A 35 mL glass vial was charged with tert-butyl(S)-4-(2-((((9H-fluoren-9-yl)methoxy)carbonyl)amino)acetamido)-5-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-A14-1) (0.28 g, 0.23 mmol) and THF (2.8 mL). To this solution, diethyl amine (0.168 g, 2.31 mmol) was added and stirred for 2 h at room temperature. After completion of reaction as indicated by TLC, reaction mixture was concentrated under vacuum and the crude was purified by trituration with diethyl ether and hexane to give title compound as yellow solid (0.19 g, 80.19%). LCMS: 1030.68 [M+H]⁺.

### Synthesis of tert-butyl(S)-4-(2-(2-bromoacetamido)acetamido)-5-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-A14-3)

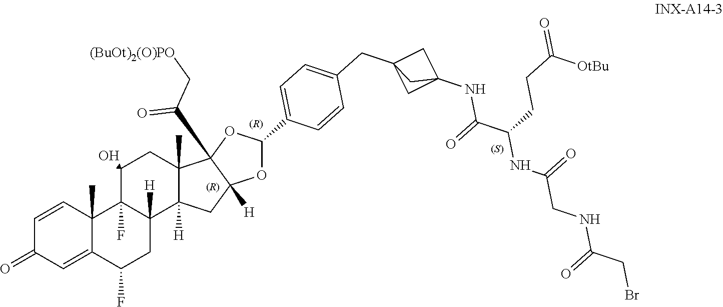

### Procedure

A 35 mL glass vial was charged with tert-butyl(S)-4-(2-aminoacetamido)-5-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-A 14-2) (0.190 g, 0.18 mmol) and DCM (2 mL). To this solution, Na₂CO₃ (0.038 g, 0.36 mmol) dissolved in water (0.4 mL) and bromoacetyl bromide (0.036 g, 0.18 mmol) were added and stirred for 1 h at room temperature. After completion of reaction as indicated by TLC, reaction mixture was quenched with water and extracted with DCM. The combined organic layer was dried over Na₂SO₄ and evaporated under vacuum to give title compound as crude sticky solid (0.15 g, 72.39%) LCMS: 1150.4 [M+H]⁺

### Synthesis of (S)-4-(2-(2-bromoacetamido)acetamido)-5-((3-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoic acid (INX-A14)

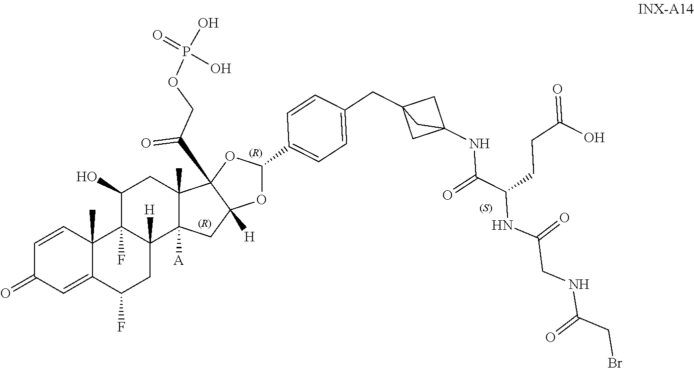

### Procedure

A 10 mL single-necked round bottom flash was charged with tert-butyl(S)-4-(2-(2-bromoacetamido)acetamido)-5-((3-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoate (INX-A14-3) (0.15 g, 0.130 mmol) and DCM (4 mL). To this solution, TFA (0.074 g, 0.65 mmol) was added and stirred for 20 min at room temperature. After completion of reaction as indicated by TLC, reaction mixture was evaporated under vacuum. The crude was purified by prep-HPLC (Column: SUNFIRE Prep C18 OBD, 19×250 mm, 5 μm, Mobile phase: A=0.05% TFA in water, B=Acetonitrile; A:B, 65:35); Retention time 12.76 min) to give title compound as white solid (0.012 g, 9.39%). LCMS: 982.3 [M+H]⁺, ¹H NMR (400 MHZ, DMSO: 0 8.55 (t, 1H), 8.35 (s, 1H), 8.04 (d, 1H), 7.35 (d, 2H), 7.25 (d, 1H), 7.10 (d, 2H), 6.30 (d, 1H), 6.13 (s, 1H), 5.80-5.60 (m, 1H), 5.50 (s, Acetal-H, 1H), 4.96 (d, J=4.8 Hz, C16H, 1H), 4.90-4.70 (m, 2H), 4.30-4.10 (m, 3H), 3.90 (s, 2H), 3.75 (d, 2H), 2.80 (d, 2H), 2.50-1.55 (m, 18H), 1.50 (s, 3H), 1.30-1.10 (m, 2H), 0.92 (s, 3H).

### Synthesis of (S)-4-(2-(2-bromoacetamido)acetamido)-5-((6-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoic acid (INX-A15)

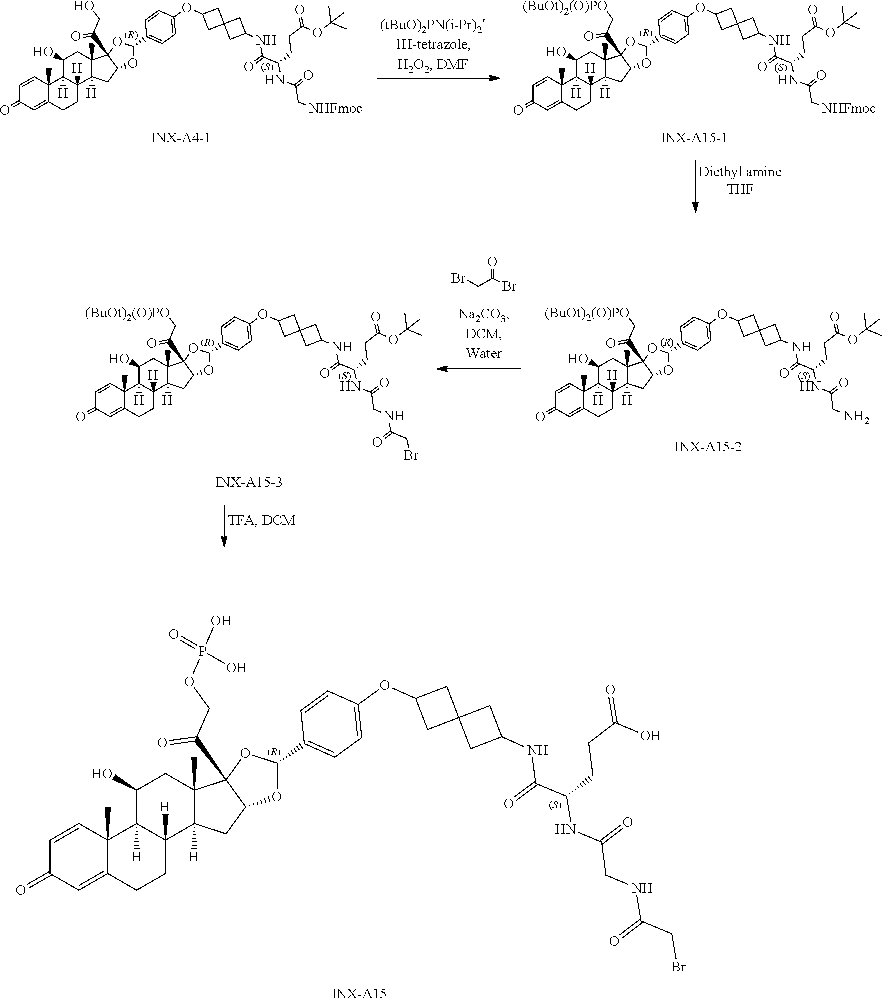

### Synthesis of tert-butyl(S)-4-(2-((((9H-fluoren-9-yl)methoxy)carbonyl)amino)acetamido)-5-((6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A15-1)

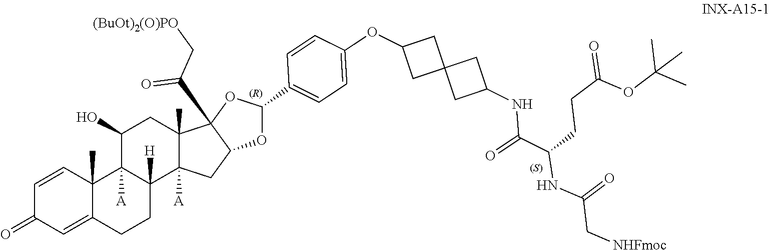

### Procedure

A 35 mL glass vial was charged with tert-butyl(S)-4-(2-((((9H-fluoren-9-yl)methoxy)carbonyl)amino)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A4-1) (0.5 g, 0.42 mmol) and DMF (2 mL). To this solution, 1H-tetrazole (0.33 g, 4.75 mmol) and (tBuO)₂PN(i-Pr)₂ (3.1 g, 11.20 mmol) were added at room temperature and stirred for 16 h. The reaction mixture was cooled to 0° C. and hydrogen peroxide (10V) was added to the reaction mixture and stirred for 1h. Reaction mixture was evaporated and purified by reverse phase column chromatography (acetonitrile:water, 80:20) to give title compound as off white solid (0.3 g, 57.30%) LCMS: 1247.50 [M+H]⁺.

### Synthesis of tert-butyl ((6aR,6bS,7S,8aS,8bS,10R,11aR,12aS,12bS)-8b-(2-((di-tert-(S)-4-(2-aminoacetamido)-5-((6-(4-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A15-2)

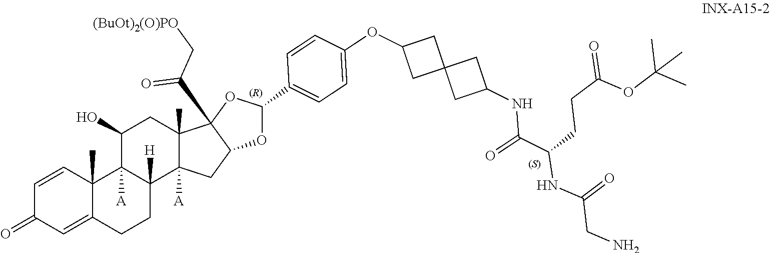

### Procedure

A 25 mL round bottom flask was charged with tert-butyl(S)-4-(2-((((9H-fluoren-9-yl)methoxy)carbonyl)amino)acetamido)-5-((6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A15-1) (0.3 g, 0.24 mmol) and THF (5 mL). To this solution, diethyl amine (0.75 g, 2.4 mmol) was added and stirred for 2 h at room temperature. After completion of reaction as indicated by TLC, reaction mixture was concentrated and purified by trituration with diethyl ether and hexane to give title compound as yellow solid (0.2 g, 81.13%) LCMS: 1024.9 [M+H]⁺.

### Synthesis of tert-butyl(S)-4-(2-(2-bromoacetamido)acetamido)-5-((6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A 15-3)

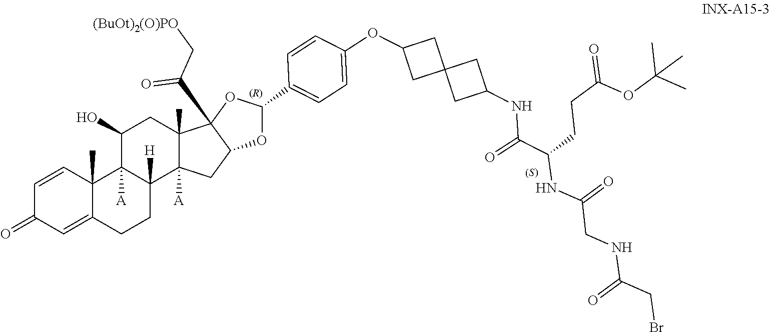

### Procedure

A 10 mL glass vial was charged with tert-butyl(S)-4-(2-aminoacetamido)-5-((6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A15-2) (0.2 g, 0.20 mmol) and DCM (4 mL). To this solution, Na₂CO₃ (0.082 g, 0.89 mmol) dissolved in water (0.3 mL) followed by bromoacetyl bromide (0.082 g, 0.45 mmol) were added to the reaction mixture and stirred for 1 h. After completion of reaction as indicated by TLC, reaction mixture was quenched with water and extracted with DCM. The combined organic layer was dried over Na₂SO₄ and evaporated under vacuum to give crude title compound INX-A15-3 product as off white solid (0.18 g, 78.59%) LCMS: 1145.4 [M+H]⁺.

### Synthesis of (S)-4-(2-(2-bromoacetamido)acetamido)-5-((6-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoic acid (INX-A15)

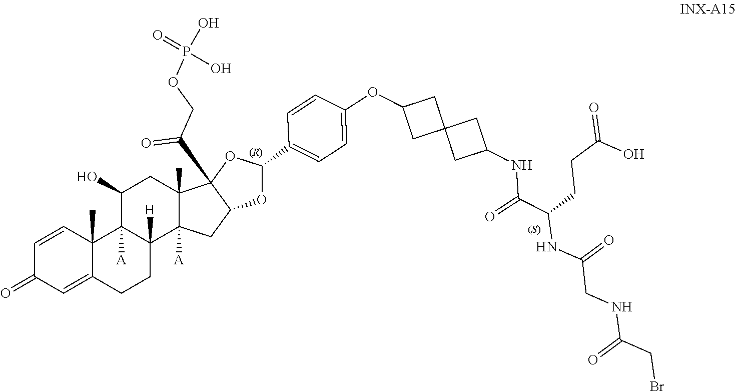

### Procedure

A 10 mL single-necked round bottom flash was charged with tert-butyl(S)-4-(2-(2-bromoacetamido)acetamido)-5-((6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A15-3) (0.18 g, 0.17 mmol) and DCM (5 mL). To this solution, TFA (0.3 mL) was added and stirred at room temperature for 1 h. After completion of reaction as indicated by TLC, reaction mixture was evaporated under vacuum to give crude product. The crude was purified by prep-HPLC (Column: YMC-PACK ODS-AQ Prep C18-S, 250×20 mm S-5 μm, 12 nm, Mobile phase: A=0.05% TFA in water, B=acetonitrile:methanol:2-propanol (65:25:10); A:B, 55:45; retention time 15.60 min) to give title compound as white solid (0.021 g, 12.64%) LCMS: 976.5 [M+H]⁺. ¹H NMR (400 MHZ, DMSO-d6, Key proton assignment): ¹H NMR (400 MHZ, DMSO: 0 7.31-7.28 (m, 3H), 6.72 (d, 2H), 6.16 (d, J=10.8 Hz, 1H), 5.92 (s, 1H), 5.41 (s, Acetal-H, 1H), 4.95 (d, C16H, 1H), 4.85-3.50 (m, 10H), 2.72-1.60 (m, 21H), 1.39 (s, 3H), 1.10-0.98 (m, 2H), 0.88 (s, 3H).

### Synthesis of (S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoic acid (INX-A16)

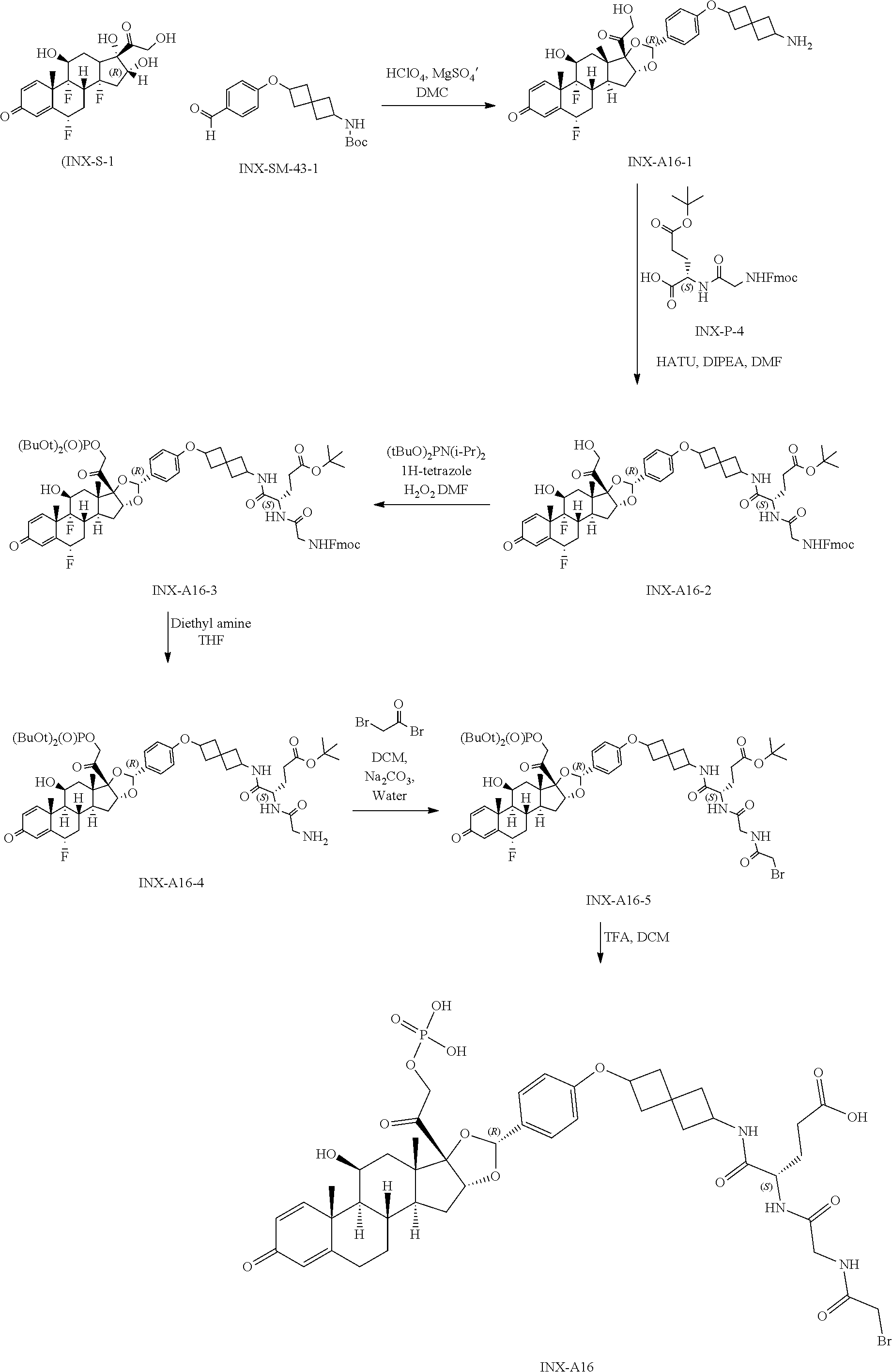

(2S,6aS,6bR,7S,8aS,8bS,10R,11aR,12aS,12bS)-10-(4-((6-Synthesis of aminospiro[3.3]heptan-2-yl)oxy) phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-A16-1)

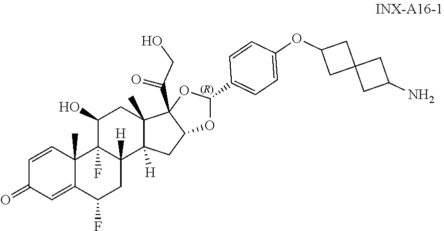

A 50 mL single necked round bottom flask was charged with tert-butyl (6-(4-formylphenoxy) spiro[3.3]heptan-2-yl)carbamate (INX-SM-43-1) (1.1 g, 3.32 mmol), (6S,8S,9R,10S,11S,13S,14S,16R,17S)-6,9-difluoro-11,16,17-trihydroxy-17-(2-hydroxyacetyl)-10, 13-dimethyl-6,7,8,9, 10, 11, 12, 13, 14, 15, 16, 17-dodecahydro-3H-cyclopenta[a]phenanthren-3-one (INX—S-1) (1.09 g, 2.65 mmol) and DCM (20 mL). To this solution, MgSO₄ (1.99 g, 16.61 mmol) followed by HClO₄ (1.6 g, 16.61 mmol) were added and stirred for 3 h at room temperature. After completion of reaction as indicated by TLC, reaction mixture was quenched with sat. NaHCO₃ solution and extracted with DCM. The combined organic layer was dried over Na₂SO₄ and evaporated under vacuum to give title compound as light yellow solid (1.3 g, crude) LCMS: 625.9 [M+H]⁺

### Synthesis of tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A16-2)

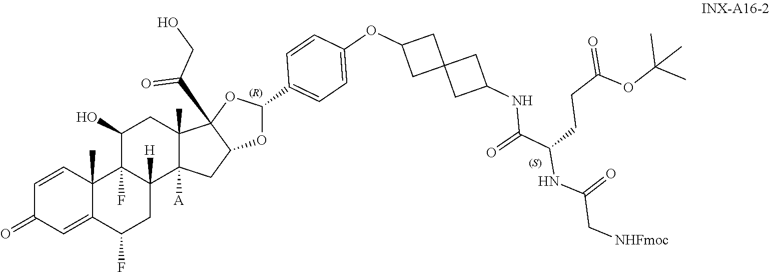

### Procedure

A 35 mL glass vial was charged with (2S,6aS,6bR,7S,8aS,8bS,10R,11aR,12aS,12bS)-10-(4-((6-aminospiro[3.3]heptan-2-yl)oxy) phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one INX-A 16-1) (1.29 g, 2.08 mmol), (S)-2-(2-((((9H-fluoren-9-yl)methoxy)carbonyl)amino)acetamido)-5-(tert-butoxy)-5-oxopentanoic acid (INX-P-4) (1.0 g, 2.08 mmol) and DMF (10 mL). To this solution HATU (0.94 g, 2.49 mmol) and DIPEA (0.89 mL, 5.20 mmol) were added to the reaction mixture and stirred for 30 min at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (water: acetonitrile 46:54) to give title compound as light yellow solid (0.8 g, 35.32%) LCMS: 1091.5 [M+H]⁺.

### Synthesis of tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A16-3)

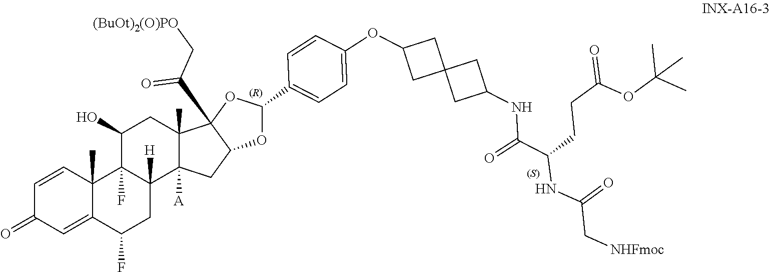

### Procedure

A 35 mL glass vial was charged with tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A16-2) (0.38 g, 0.32 mmol) and DMF (1.5 mL). To this solution, 1H-tetrazole (0.24 g, 3.40 mmol) and (tBuO)₂PN(i-Pr)₂ (2.3 g, 8.21 mmol) were added and stirred for 16 h at room temperature. The reaction mixture was cooled at 0° C. and hydrogen peroxide (4 mL) was added into the reaction mixture and stirred 1 hr at room temperature. The reaction mixture was subjected to reverse phase column chromatography (acetonitrile:water, 80:20) to give title compound as off white solid (0.2 g, 48.73%) LCMS: 1283.7 [M+H]⁺.

### Synthesis of tert-butyl (S)-4-(2-aminoacetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A 16-4)

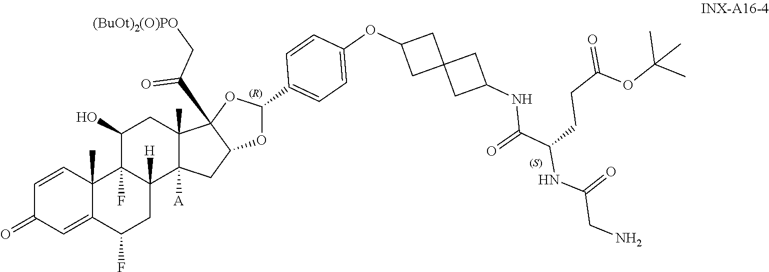

### Procedure

A 25 mL single necked round bottom flask was charged with tert-butyl(S)-4-(2-((((9H-fluoren-9-yl)methoxy)carbonyl)amino)acetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A16-3) (0.17 g, 0.13 mmol) and THF (3 mL). To this solution, diethyl amine (0.096 g, 1.3 mmol) was added and stirred for 2 h at room temperature. After completion of reaction as indicated by TLC, reaction mixture was concentrated under vacuum. The crude was triturated with diethyl ether and hexane to give title compound as yellow solid (0.13 g, 94.32%). LCMS: 1060.8 [M+H]⁺

### Synthesis of tert-butyl(S)-4-(2-(2-bromoacetamido)acetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A16-5)

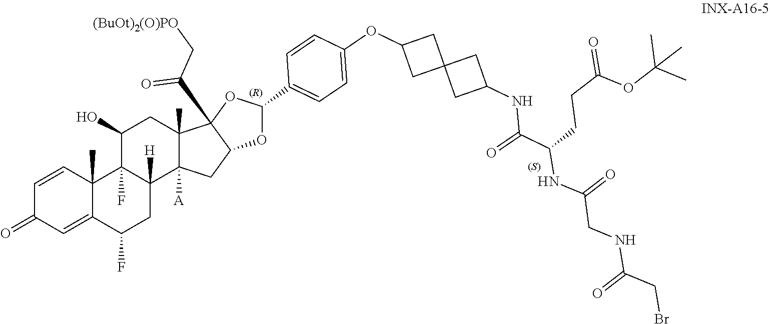

### Procedure

A 10 mL glass vial was charged with tert-butyl(S)-4-(2-aminoacetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A 16-4) (0.13 g, 0.12 mmol) and DCM (3 mL). To this solution, Na₂CO₃ (0.051 g, 0.49 mmol) dissolved in water (0.3 mL) followed by bromoacetyl bromide (0.049 g, 0.24 mmol) were added and stirred for 1 h at room temperature. After completion of reaction as indicated by TLC, reaction mixture was quenched with water and extracted with DCM. The combined organic layer was dried over Na₂SO₄ and evaporated under vacuum to give crude title compound INX-A16-5 product as off white solid (0.12 g, 84.66%). LCMS 1182.1 [M+H]⁺.

### Synthesis of (S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoic acid (INX-A16)

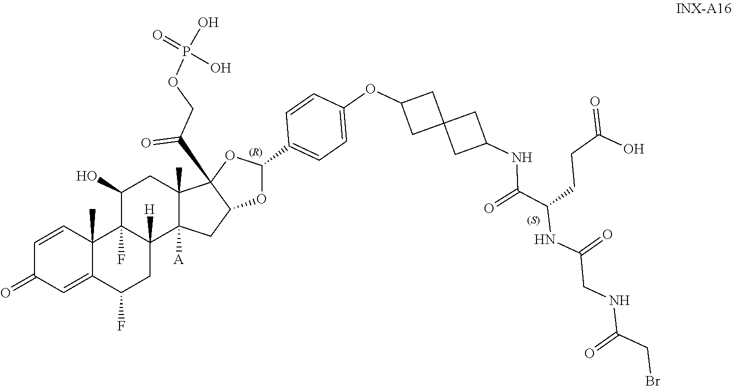

### Procedure

A 10 mL single-necked round bottom flash was charged with tert-butyl(S)-4-(2-(2-bromoacetamido)acetamido)-5-((6-(4-((2S,6aS,6bR,7S,8aS,8bS,10R,11aR,12aS,12bS)-8b-(2-((di-tert-butoxyphosphoryl)oxy)acetyl)-2,6b-difluoro-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl) phenoxy) spiro[3.3]heptan-2-yl)amino)-5-oxopentanoate (INX-A 16-5) (0.12 g, 0.10 mmol) and DCM (2 mL). To this solution, TFA (0.057 g, 0.50 mmol) was added and stirred for 1 h at room temperature. After completion of reaction as indicated by TLC, reaction mixture was evaporated under vacuum. The crude was purified by prep-HPLC (Column: SUNFIRE Prep C18 OBD (250×19) mm, 5 μm, Mobile phase: A=0.05% TFA in water, B=acetonitrile; A:B=64:36; retention time 12.86 min) to give title compound as white solid (0.013 g, 12.83%) LCMS: 1012.4 [M+H]⁺. ¹H NMR (400 MHZ, DMSO: δ: 8.54 (t, 1H), 8.11-8.09 (m, 2H), 7.33-7.25 (m, 3H), 6.84 (d, J=8.4 Hz, 2H), 6.30 (dd, J=10.4 & 1.6 Hz, 1H), 6.13 (s, 1H), 5.72-5.60 (m, CH—F, 1H), 5.48 (s, Acetal-H, 1H), 4.93 (d, J=4.8 Hz, C16H, 1H), 4.90-4.91 (m, 1H), 4.61-4.57 (m, 2H), 4.30-4.00 (m, 5H), 3.94 (s, 2H), 3.00-1.67 (m, 20H), 1.50-1.48 (m, 3H), 0.89 (s, 3H)

### Synthesis of (6aR,6bS,7S,8aS,8bS,10R,11aR,12aS,12bS)-10-(4-((2-azaspiro[3.3]heptan-6-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-47)

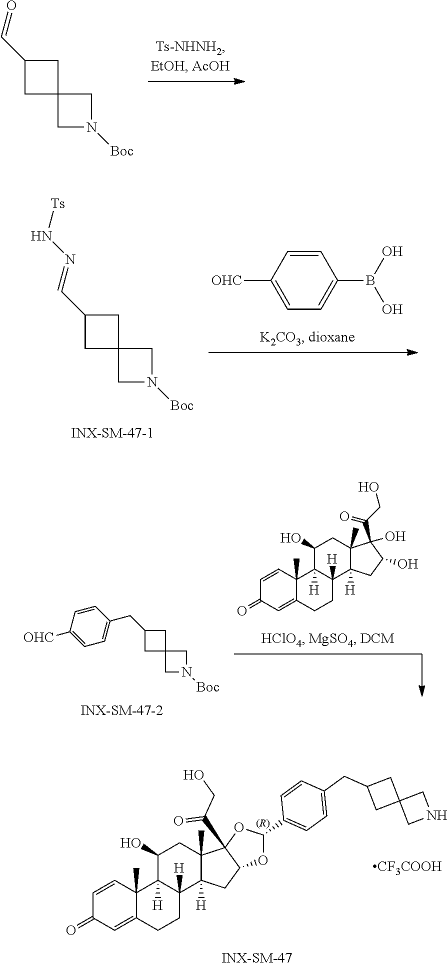

### Step-1: Synthesis of tert-butyl (E)-6-((2-tosylhydrazono)methyl)-2-azaspiro[3.3]heptane-2-carboxylate (INX-SM-47-1)

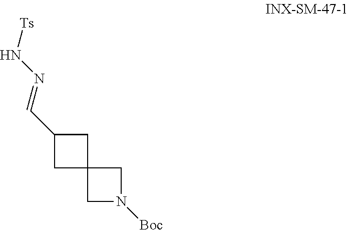 [SMILES: Cc1ccc(S(=O)(=O)N/N=C/C2CC3(C2)CN(C(=O)OC(C)(C)C)C3)cc1]

### Procedure

A 35 mL glass vial was charged with tert-butyl 6-formyl-2-azaspiro[3.3]heptane-2-carboxylate (0.2 g, 0.88 mmol) and EtOH (10 mL) under nitrogen. To this solution, p-toluenesulfonhydrazide (0.247 g, 1.333 mmol) and catalytic amount of AcOH (0.1 mL) were added and stirred for 0.5h at room temperature. After completion of reaction as indicated by TLC, reaction mixture was poured into water and the solid was filtered and dried under vacuum to give title compound as white solid (0.3 g, 86.6%). LCMS: 394.2 [M+H]⁺

### Synthesis of tert-butyl 6-(4-formylbenzyl)-2-azaspiro[3.3]heptane-2-carboxylate (INX-SM-47-2)

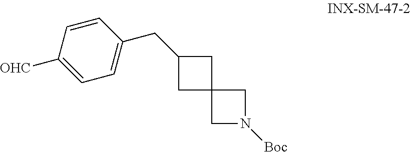 [SMILES: CC(C)(C)OC(=O)N1CC2(CC(Cc3ccc(C=O)cc3)C2)C1]

### Procedure

A 35 mL glass vial was charged with tert-butyl (E)-6-((2-tosylhydrazono)methyl)-2-azaspiro[3.3]heptane-2-carboxylate (INX-SM-47-1) (0.3 g, 0.762 mmol) and dioxane (5 mL) under nitrogen. To this solution, (4-formylphenyl)boronic acid (0.171 g, 1.143 mmol) and K₂CO3 (0.158 g, 1.143 mmol) were added and reaction mixture was allowed to stir for 3 h at 100° C. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane, 50:50) to give title compound as yellow sticky solid (0.065 g, 27.03%). LCMS: 316.2 [M+H]⁺

### Synthesis of (6aR,6bS,7S,8aS,8bS,10R,11aR,12aS,12bS)-10-(4-((2-azaspiro[3.3]heptan-6-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-47)

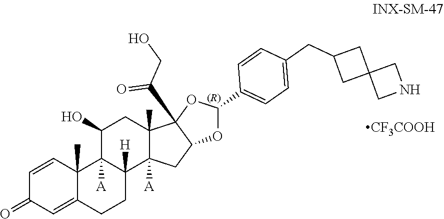

### Procedure

A 25 mL single-necked round bottom flash was charged with tert-butyl 6-(4-formylbenzyl)-2-azaspiro[3.3]heptane-2-carboxylate (INX-SM-47-2) (0.065 g, 0.206 mmol) and (8S,9S,10R,11S,13S,14S,16R,17S)-11,16,17-trihydroxy-17-(2-hydroxyacetyl)-10, 13-dimethyl-6,7,8,9,10,11,12,13,14,15, 16, 17-dodecahydro-3H-cyclopenta[a]phenanthren-3-one (16-alpha-hydroxyprednisolone) (0.077 g, 0.206 mmol) in DCM (2 mL). To this solution, MgSO₄ (0.103 g, 1.03 mmol) and HClO4 (0.123 g, 1.03 mmol) were added and stirred for another 1.5h at room temperature. After completion of reaction as indicated by TLC, reaction mixture was quenched with sat. NaHCO₃ solution and concentrated under vacuum. The crude was triturated with cold water and solid was filtered. The crude was purified by prep-HPLC (Column: YMC-PACK ODS-AQ Prep C18-S, 250×20 mm S-5 μm, 12 nm, Mobile phase: A=0.05% TFA in water, B=acetonitrile:methanol:2-propanol (65:25:10), A:B=58:42, retention time 12.23 min) to give title compound as white solid (0.025 g, 17.77%). LCMS: 574.4 [M+H]⁺; ¹H NMR (400 MHZ, MeOD: δ: 7.47 (d, J=10.0 Hz, 1H), 7.37 (d, J=8.0 Hz, 2H), 7.18 (d, J=8.4 Hz, 2H), 6.27 (dd, J=10.0 & 2.0 Hz, 1H), 6.04 (s, 1H), 5.46 (s, Acetal-H, 1H), 5.06 (d, J=5.2 Hz, C16H, 1H), 4.65-4.60 (m, 2H), 4.44 (d, J=2.8 Hz, 1H), 4.35-4.31 (m, 1H), 4.03 (s, 2H), 3.95 (s, 2H), 2.70-1.60 (m, 15H), 1.50 (s, 3H), 1.17-1.04 (m, 2H), 1.01 (s, 3H)

### Synthesis of (6aR,6bS,7S,8aS,8bS,10R,11aR,12aS,12bS)-7-hydroxy-8b-(2-hydroxyacetyl)-6a,8a-dimethyl-10-(4-((3-(methylamino) cyclobutyl)methyl)phenyl)-1,2,6a,6b,7,8,8a,8b,11a,12,12a,12b-dodecahydro-4H-naphtho[2′,1′:4,5]indeno[1,2-d][1,3]dioxol-4-one (INX-SM-49)

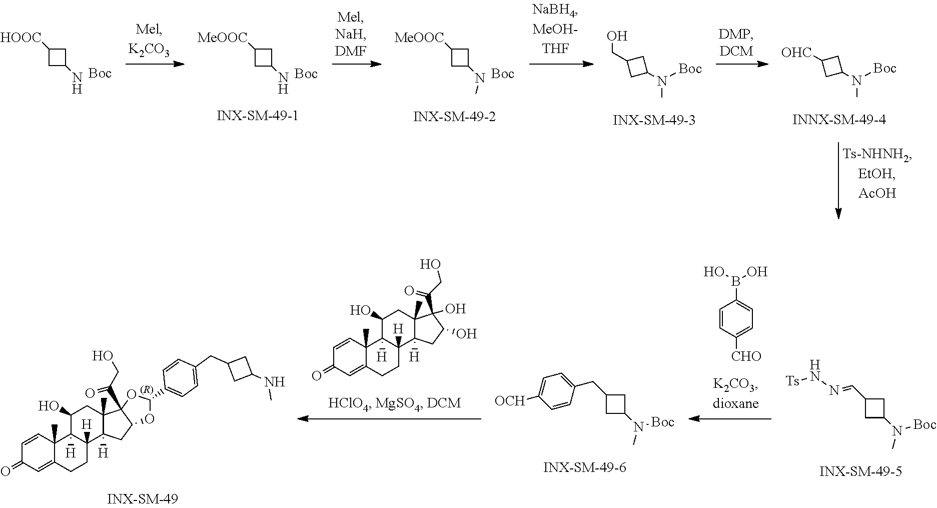

### Synthesis of methyl 3-((tert-butoxycarbonyl)amino)cyclobutane-1-carboxylatecarbamate (INX-SM-49-1)

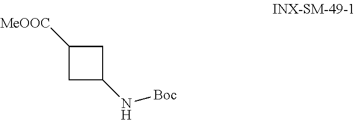 [SMILES: COC(=O)C1CC(NC(=O)OC(C)(C)C)C1]

### Procedure

A 100 mL single necked round bottom flask was charged 3-((tert-butoxycarbonyl)amino)cyclobutane-1-carboxylic acid (3.0 g, 13.95 mmol), potassium carbonate (3.8 g, 27.90 mmol) and DMF (20 mL). To this solution, methyl iodide (2.9 g, 20.92 mmol) was added and stirred for another 1 h at room temperature. After completion of reaction as indicated by TLC, reaction mixture was dilute with H₂O and extracted with ethyl acetate. The combined organic layer was dried over Na₂SO₄ and evaporated under vacuum to give title compound as yellow solid (2.2 g, 68.85%). LCMS: 230.20 [M+H]⁺

### Synthesis of methyl 3-((tert-butoxycarbonyl)(methyl)amino)cyclobutane-1-carboxylate (INX-SM-49-2)

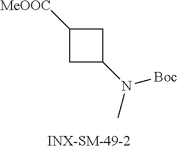 [SMILES: COC(=O)C1CC(N(C)C(=O)OC(C)(C)C)C1]

### Procedure

A 100 mL single necked round bottom flask was charged with methyl 3-((tert-butoxycarbonyl)amino)cyclobutane-1-carboxylatecarbamate (INX-SM-49-1) (2.2 g, 9.60 mmol) and 60% sodium hydride (0.77 g, 19.21 mmol) in DMF (20 mL) at 0° C. To this solution, methyl iodide (2.03 g, 14.40 mmol) was added and stirred for another 16 h at room temperature. After completion of reaction as indicated by TLC, reaction mixture was quenched with water and extracted with ethyl acetate. The combined organic layer was dried over Na₂SO₄ and evaporated under vacuum to give title compound as yellow solid (1.8 g, 77.0%). LCMS: 244.20 [M+H]⁺

### Synthesis of tert-butyl (3-(hydroxymethyl)cyclobutyl)(methyl)carbamate (INX-SM-49-3)

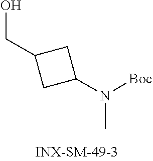

### Procedure

A 100 mL single necked round bottom flask was charged with methyl 3-((tert-butoxycarbonyl)(methyl)amino)cyclobutane-1-carboxylate (INX-SM-49-2) (1.8 g, 7.40 mmol) in THF:MeOH (1:1, 20 mL) was added sodium borohydride (1.3 g, 37.0 mmol) and stirred for another 5 h at room temperature. After completion of reaction as indicated by TLC, reaction mixture was diluted with water and extracted with ethyl acetate. The combined organic layer was dried over Na₂SO₄ and evaporated under vacuum to give title compound as gummy solid (1.7 g, crude). LCMS: 216.1 [M+H]⁺

### Synthesis of tert-butyl (3-formylcyclobutyl)(methyl)carbamate (INX-SM-49-4)

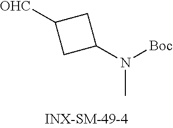

### Procedure

A 100 mL single necked round bottom flask was charged with tert-butyl (3-(hydroxymethyl)cyclobutyl)(methyl)carbamate (INX-SM-49-3) (1.7 g, crude) in DCM (20 mL). To this solution, DMP (5.02 g, 11.80 mmol) was added at room temperature and stirred for 2 h. After completion of reaction as indicated by TLC, reaction mixture was quenched with sat. NaHCO₃ solution and extracted with dichloromethane. The combined organic layer was dried over Na₂SO₄ and evaporated under vacuum to give title compound as gummy solid (1.5 g, 89.06%). Crude was immediately used for next step.

### Synthesis of tert-butyl (E)-methyl (3-((2-tosylhydrazono)methyl)cyclobutyl)carbamate (INX-SM-49-5)

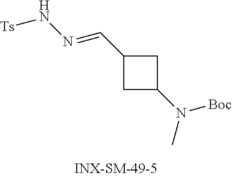 [SMILES: Cc1ccc(S(=O)(=O)N/N=C/C2CC(N(C)C(=O)OC(C)(C)C)C2)cc1]

### Procedure

A 10 mL glass vial was charged with tert-butyl (3-formylcyclobutyl)(methyl)carbamate (INX-SM-49-4) (1.5 g, 7.04 mmol) and ethanol (20 mL). To this solution, p-Toluenesulfonyl hydrazide (1.4 g, 7.7 mmol) and acetic acid (catalytic) were added and stirred for 1 h at room temperature. After completion of reaction as indicated by TLC, reaction mixture was poured into water and the resulted solid was filtered and dried under vacuum to give title compound as white solid (2.0 g 74.46%). LCMS: 382.22 [M+H]⁺

### Synthesis of tert-butyl (3-(4-formylbenzyl)cyclobutyl)(methyl)carbamate (INX-SM-49-6)

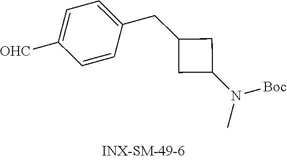 [SMILES: CN(C(=O)OC(C)(C)C)C1CC(Cc2ccc(C=O)cc2)C1]

A 50 mL single-necked round bottom flask was charged with tert-butyl (E)-methyl (3-((2-tosylhydrazono)methyl)cyclobutyl)carbamate (INX-SM-49-5) (2.0 g, 5.24 mmol) and dioxane (20 mL). To this solution, (4-formylphenyl)boronic acid (1.17 g, 7.87 mmol) and K2CO3 (1.08 g, 7.87 mmol) were added and stirred for another 2 h at 110° C. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane: 1:1) to give title compound as colorless liquid (0.500 g, 31.45%). LCMS: 304.2 [M+H]⁺

### Synthesis of (6aR,6bS,7S,8aS,8bS,10R,11aR,12aS,12bS)-7-hydroxy-8b-(2-hydroxyacetyl)-6a,8a-dimethyl-10-(4-((3-(methylamino) cyclobutyl)methyl)phenyl)-1,2,6a,6b,7,8,8a,8b,11a,12,12a,12b-dodecahydro-4H-naphtho[2′,1′:4,5]indeno[1,2-d][1,3]dioxol-4-one (INX-SM-49)

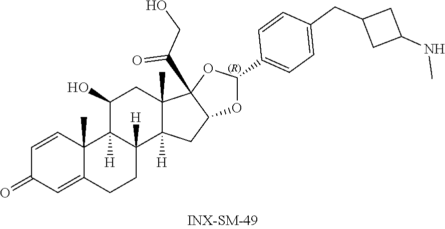

### Procedure

A 25 mL single-necked round bottom flask was charged with tert-butyl (3-(4-formylbenzyl)cyclobutyl)(methyl)carbamate (INX-SM-49-6) (0.16 g, 0.52 mmol), (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthren-3-one (16-alpha-hydroxyprednisolone) (0.21 g, 0.58 mmol) and DCM (2 mL). To this solution, MgSO₄ (0.31 g, 2.6 mmol) and HClO₄ (0.26 g, 2.6 mmol) were added and stirred for another 2 h at room temperature. After completion of reaction as indicated by TLC, reaction mixture was quenched with sat. NaHCO₃ solution and extracted with MDC. The combined organic layer was dried over Na₂SO₄ and evaporated under vacuum to give crude product. The crude was purified by prep-HPLC to give title compound as white solid (0.050 g, 17.11%). LCMS: 562.4 [M+H]⁺; ¹H NMR (400 MHZ, DMSO-d6-d20: δ: 7.37 (d, J=8.4 Hz, 2H), 7.32 (d, J=10.0 Hz, 1H), 7.17 (d, J=8.0 Hz, 2H), 6.17 (dd, J=10 & 1.6 Hz, 1H), 5.94 (s, 1H), 5.41 (s, Acetal-H, 1H), 4.93 (d, J=4.8 Hz, C16H, 1H), 4.48 (m, 1H), 4.31-4.29 (m, 1H), 4.21-4.15 (m, 1H), 3.50-3.40 (m, 1H), 2.69-1.60 (m, 19H), 1.40 (s, 3H), 0.99-0.96 (m, 2H), 0.87 (s, 3H).

### Synthesis of (4S)-4-(2-(2-bromoacetamido)acetamido)-5-(((2S,3R,4R,5S)-4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) cuban-1-yl)amino)-5-oxopentanoic acid (INX-A2)

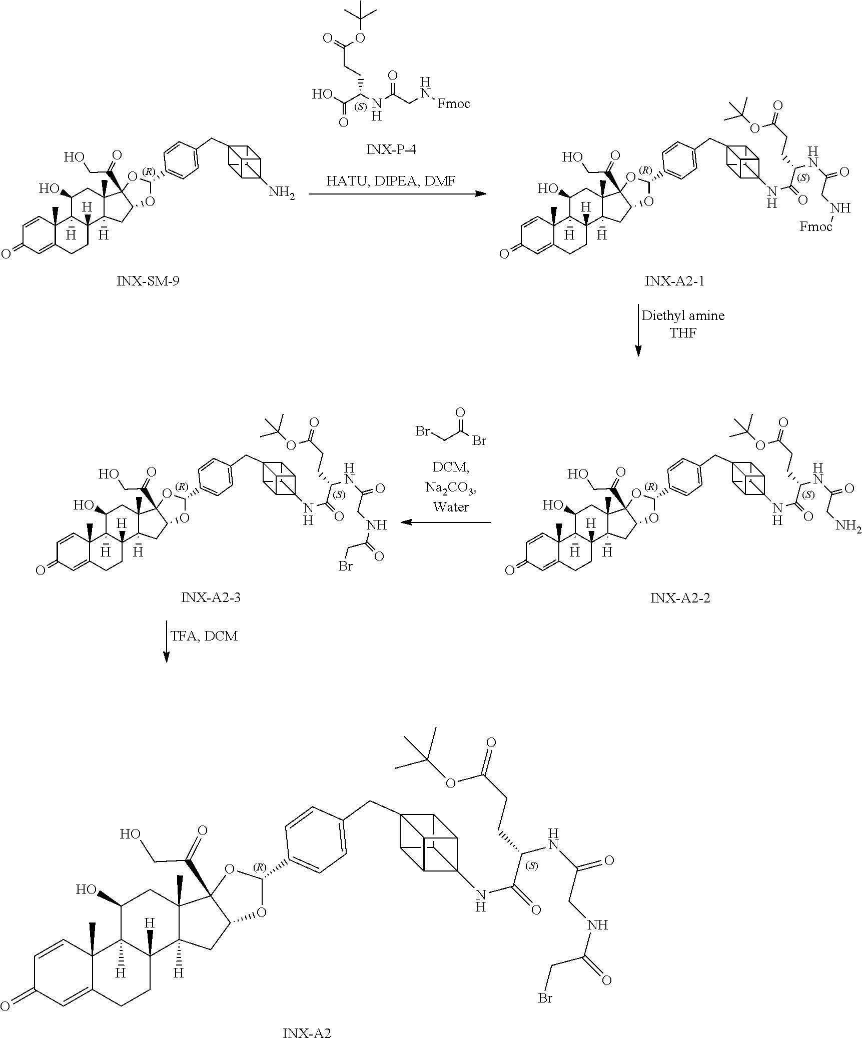

### Synthesis of tert-butyl yl)methoxy)carbonyl)amino)acetamido)-5-(((2S,3R,4R,5S)-4-(4-(4S)-4-(2-((((9H-fluoren-9-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) cuban-1-yl)amino)-5-oxopentanoate (INX-A2-1)

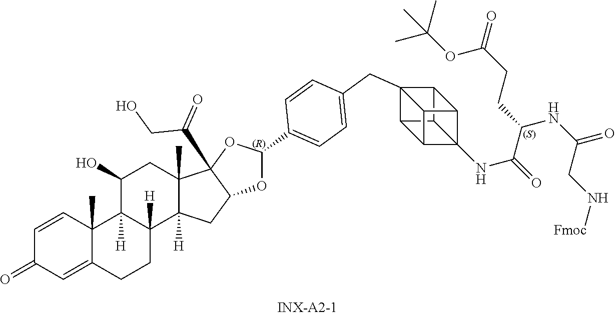

### Procedure

A 25 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) 0.137 g, 0.285 mmol), HATU (0.163 g, 0.428 mmol) and DMF (2 mL). To this solution, DIPEA (0.110 g, 0.857 mmol) and (6aR,6bS,7S,8aS,8bS,11aR,12aS,12bS)-10-(4-(((1s,2R,3S,8S)-4-aminocuban-1-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-9) (0.160 g, 0.0.285 mmol) were added and stirred the reaction mixture for 2 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography to give title compound as pale yellow solid (0.08 g, 26.39%). LCMS: 1060.7 [M+H]⁺

### Synthesis of tert-butyl (4S)-4-(2-aminoacetamido)-5-(((2S,3R,4R,5S)-4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) cuban-1-yl)amino)-5-oxopentanoate (INX-A2-2)

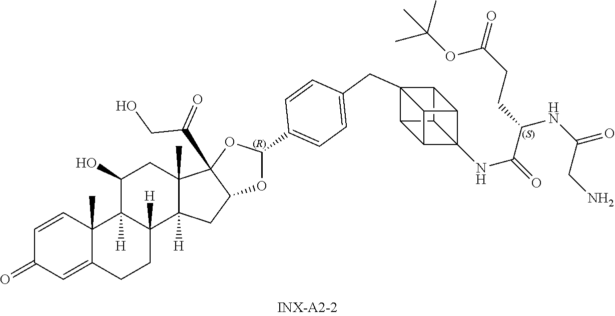

### Procedure

A 25 mL single-necked round bottom flash was charged with tert-butyl (4S)-4-(2-((((9H-fluoren-9-yl)methoxy)carbonyl)amino)acetamido)-5-(((2S,3R,4R,5S)-4-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) cuban-1-yl)amino)-5-oxopentanoate (INX-A2-1) (0.08 g, 0.075 mmol) and THF (3 mL). To this solution, diethyl amine (0.055 g, 0.75 mmol) and 1,8-Diazabicyclo (5.4.0) undec-7-ene (DBU) (catalytic) were added and stirred for 2 h at room temperature. After completion of reaction as indicated by TLC, reaction mixture was concentrated under vacuum. The crude was triturated with DCM and hexane to give title compound as white solid (0.06 g, 95.46%). LCMS: 839.4 [M+H]⁺.

### Synthesis of tert-butyl (4S)-4-(2-(2-bromoacetamido)acetamido)-5-(((2S,3R,4R,5S)-4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) cuban-1-yl)amino)-5-oxopentanoate (INX-A2-3)

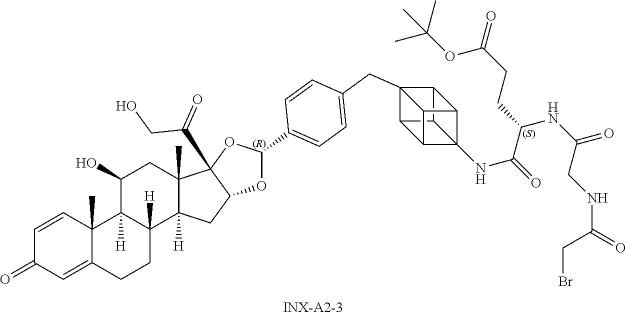

### Procedure

A 25 mL single-necked round bottom flash was charged with tert-butyl (4S)-4-(2-((((9H-fluoren-9-yl)methoxy)carbonyl)amino)acetamido)-5-(((2S,3R,4R,5S)-4-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) cuban-1-yl)amino)-5-oxopentanoate (INX-A2-2) (0.09 g, 0.107 mmol) and DCM (2 mL). To this solution, Na₂CO₃ (0.022 g, 0.21 mmol) dissolved in water (0.1 mL) followed by bromoacetyl bromide (0.043 g, 0.021 mmol) were added at room temperature and stirred for 1 h. After completion of reaction as indicated by TLC, reaction mixture was quenched with water and extracted with DCM. The combined organic layer was dried over Na₂SO₄ and evaporated under vacuum to give title compound as off-white solid (0.065 g, 63.11%).

LCMS: calculated for C50H61⁷⁹BrN₃O₁₁ (958.35), found 958.2 [M+H]⁺.

### Synthesis of (4S)-4-(2-(2-bromoacetamido)acetamido)-5-(((2S,3R,4R,5S)-4-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) cuban-1-yl)amino)-5-oxopentanoic acid (INX-A2)

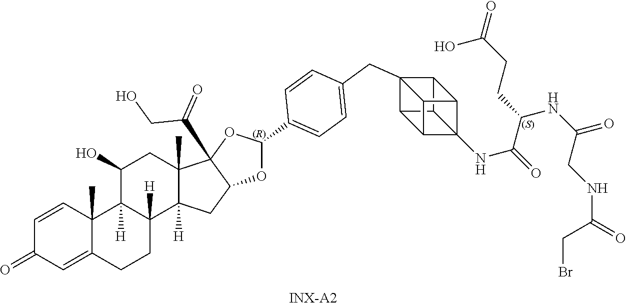

### Procedure

A 25 mL single-necked round bottom flash was charged with tert-butyl (4S)-4-(2-(2-bromoacetamido)acetamido)-5-(((2S,3R,4R,5S)-4-(4-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) cuban-1-yl)amino)-5-oxopentanoate (INX-A2-3) (0.065 g, 0.067 mmol) and DCM (3 mL). To this solution, TFA (0.5 mL) and triisopropyl saline (catalytic) were added and stirred for 2 h at room temperature. After completion of reaction as indicated by TLC, reaction mixture was evaporated under vacuum to give crude title compound product as off white solid (0.055 g crude). LCMS 903.5 [M+H]⁺.

### Synthesis of (S)-4-(2-(2-(((R)-2-amino-2-carboxyethyl) thio) acetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoic acid (INX—P—CYS)

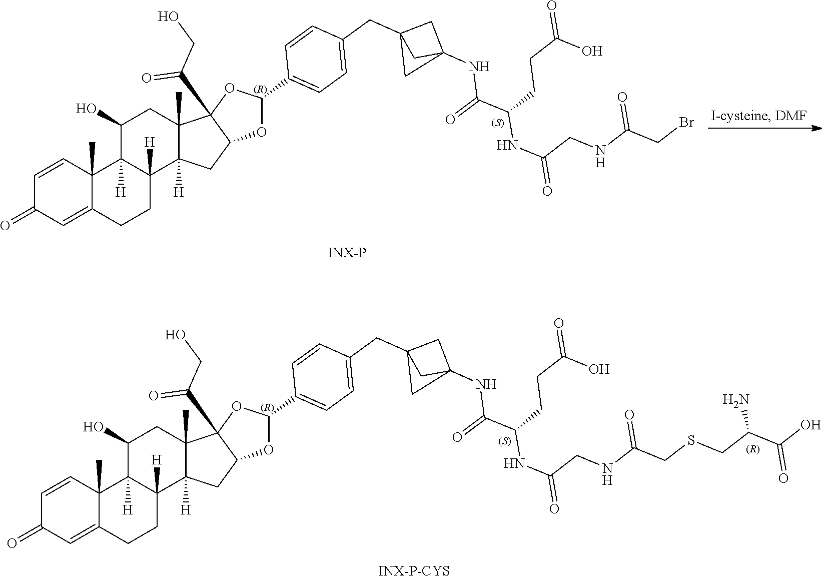

### Procedure

A 10 mL single-necked round bottom flash was charged with(S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-5-oxopentanoic acid (INX—P—CYS) (0.1 g, 0.11 mmol) and DMF (1 mL). To this solution, L-cysteine (0.003 g, 0.12 mmol) was added and stirred for 2 h at room temperature. After completion of reaction as indicated by LCMS, reaction mixture was lyophilized and the crude was purified by reverse phase column chromatography (acetonitrile:water, 45:55) to give title compound as off white solid (0.050 g, 50.11%). LCMS: 907.5 [M+H]⁺; ¹H NMR (400 MHz, MeOD: δ: 7.47 (d, J=10.4 Hz, 1H), 7.38 (d, J=8.0 Hz, 2H), 7.14 (d, J=8.0 Hz, 2H), 6.28 (d, J=10.0 Hz, 1H), 6.05 (s, 1H), 5.46 (s, Acetal-H, 1H), 5.06 (d, J=5.2 Hz, C16H, 1H), 4.65 (d, 1H), 4.46 (m, 1H), 4.35 (d, 1H), 4.18-4.16 (m, 1H), 3.90 (m, 2H), 3.79 (m, 1H), 3.50-3.00 (m, 4H), 3.00-1.60 (m, 21H), 1.52 (s, 3H), 1.30-1.07 (m, 2H), 1.01 (s, 3H).

### Synthesis of (S)-4-(2-(2-(((R)-2-amino-2-carboxyethyl) thio) acetamido)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid compound with 2,2,2-trifluoroacetic acid (INX—V—CYS)

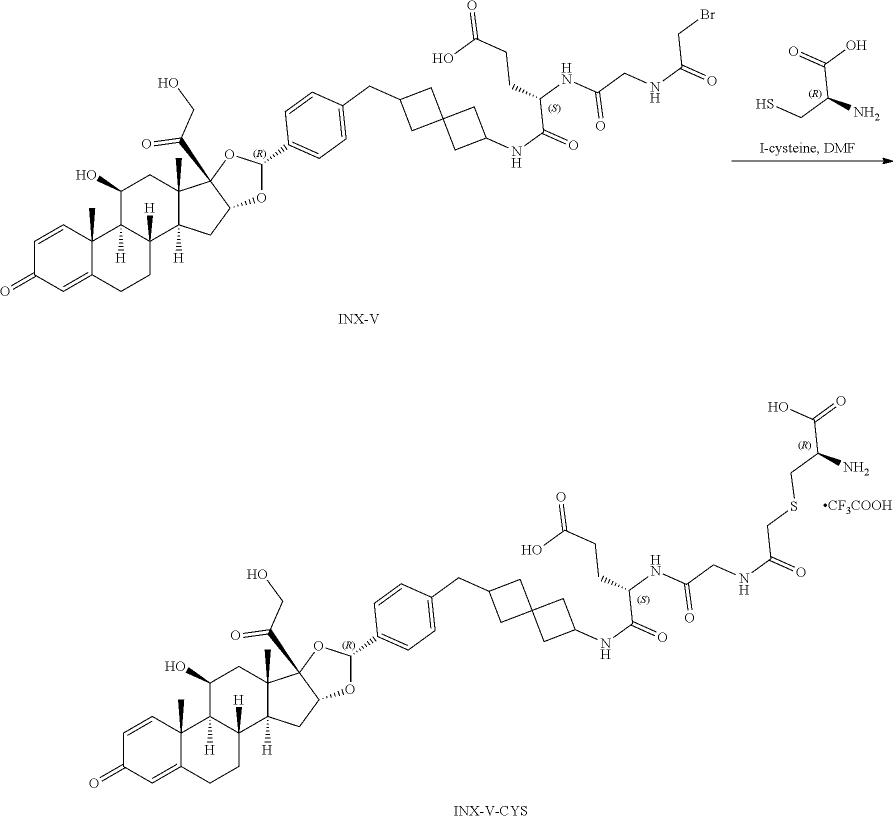

A 10 mL single-necked round bottom flash was charged with(S)-4-(2-(2-bromoacetamido)acetamido)-5-((6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX—V) (0.1 g, 0.11 mmol) and DMF (1.5 mL). To this solution, L-cysteine (0.0027 g, 0.223 mmol) was added and stirred for 16 h at room temperature. After completion of reaction as indicated by LCMS, reaction mixture was lyophilized. The isolated crude was purified by prep-HPLC (Column: YMC-Actus Triart Prep C18-S, 250×20 mm S-5 μm, 12 nm, Mobile phase: A=0.05% TFA in water, B=MTBE: Acetonitrile (10:90), A:B=75:25), retention time 12.5 min to give title compound as white solid (0.012 g, 10.24%). LCMS: 935.5 [M+H]⁺;

¹H NMR (400 MHZ, MeOD: δ: 7.46 (d, J=10.4 Hz, 1H), 7.34 (d, J=8.0 Hz, 2H), 7.15 (d, J=8.0 Hz, 2H), 6.27 (dd, J=1.4 Hz, 10 Hz, 1H), 6.04 (s, 1H), 5.45 (s, Acetal-H, 1H), 5.06 (d, J=5.2 Hz, C16H, 1H), 4.66 (d, 1H), 4.45-4.40 (m, 1H), 4.36-4.31 (m, 2H), 4.12 (m, 1H), 4.00-3.92 (m, 3H), 3.50-1.73 (m, 28H), 1.52 (s, 3H), 1.20-1.03 (m, 2H), 1.00 (s, 3H).

### Synthesis of S-(2-((2-(((S)-6-amino-1-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl)amino)-1-oxohexan-2-yl)amino)-2-oxoethyl)amino)-2-oxoethyl)-L-cysteine compound with 2,2,2-trifluoroacetic acid (INX—W—CYS)

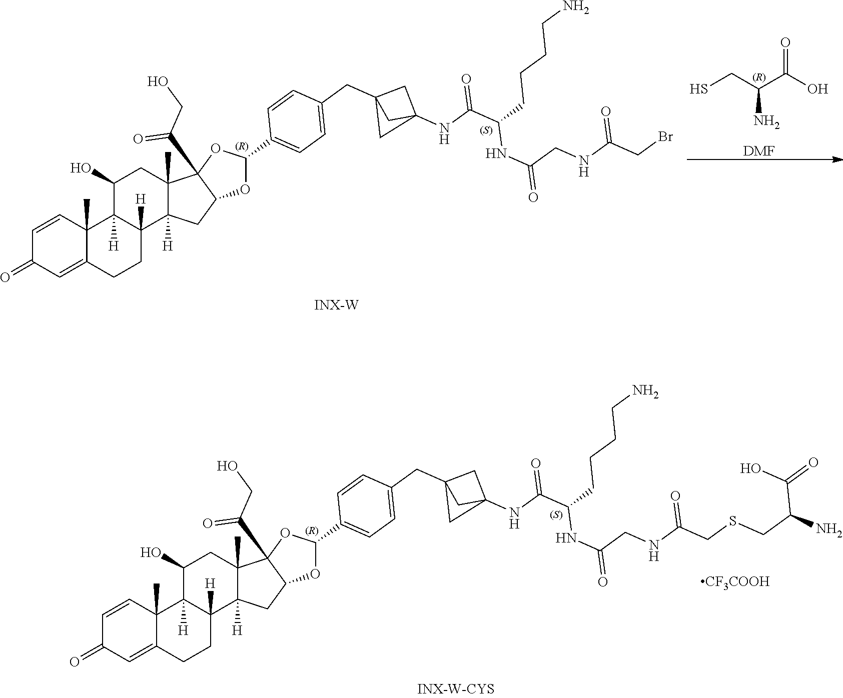

### Procedure

A 10 mL single-necked round bottom flash was charged with ((S)-6-amino-2-(2-(2-bromoacetamido)acetamido)-N-(3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)bicyclo[1.1.1]pentan-1-yl) hexanamide (INX—W) (0.2 g, 0.23 mmol) and DMF (1 mL). To this solution, L-cysteine (0.041 g, 0.34 mmol) was added and stirred for 2 h at room temperature. After completion of reaction as indicated by LCMS, reaction mixture was lyophilized. The crude was purified by prep HPLC (Column: SUNFIRE Prep Silica, OBD, 150-19 mm, 5 μm, Mobile phase: A=0.05% TFA in water, B=acetonitrile, A:B=76:24), retention time 19.5 min) to give title compound as off white solid (0.020 g, 8.49%). LCMS: 906.4 [M+H]⁺;

¹H NMR (400 MHZ, MeOD: δ: 7.47 (d, J=10.4 Hz, 1H), 7.38 (d, J=8.0 Hz, 2H), 7.14 (d, J=8.0 Hz, 2H), 6.28 (d, 1H), 6.05 (s, 1H), 5.48 (s, Acetal-H, 1H), 5.07 (d, J=5.2 Hz, C16H, 1H), 4.67 (d, 1H), 4.50-4.32 (m, 3H), 3,39 (s, 2H), 3.40-3.00 (m, 2H), 3.00-2.90 (m, 2H), 2.85 (s, 2H), 2.75-1.60 (m, 22H), 1.52 (s, 3H), 1.50-1.35 (m, 2H), 1.20-1.05 (m, 2H), 1.01 (s, 3H).

### Synthesis of (6aR,6bS,7S,8aS,8bS,11aR,12aS,12bS)-10-(4-((3-aminobicyclo[1.1.1]pentan-1-yl)methyl)phenyl)-7-hydroxy-8b-(2-hydroxyacetyl)-6a,8a-dimethyl-1,6a,6b,7,8,8a,8b,9,10,11,11a,12,12a,12b-tetradecahydronaphtho[2′,1′:4,5]indeno[1,2-c]pyrrol-4 (2H)-one 2,2,2-trifluoroacetate (INX-SM-34); and

### (6aR,6bS,7S,8aS,8bS,11aR,12aS,12bS)-10-(4-((3-aminobicyclo[1.1.1]pentan-1-yl)methyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrole-8b(2H)-carboxylic acid compound with 2,2,2-trifluoroacetic acid (INX-SM-42)

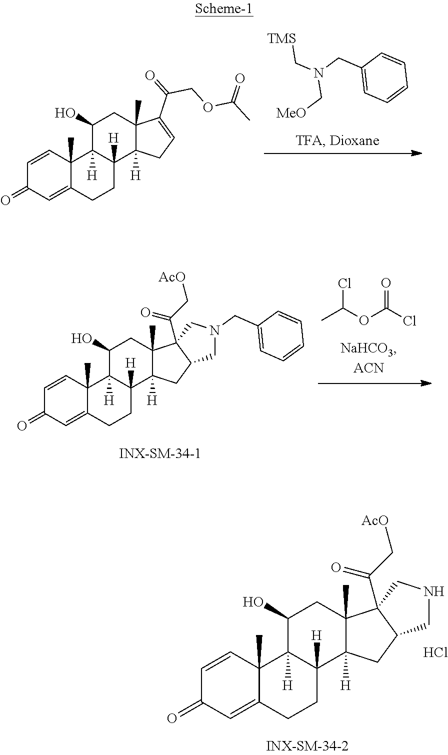

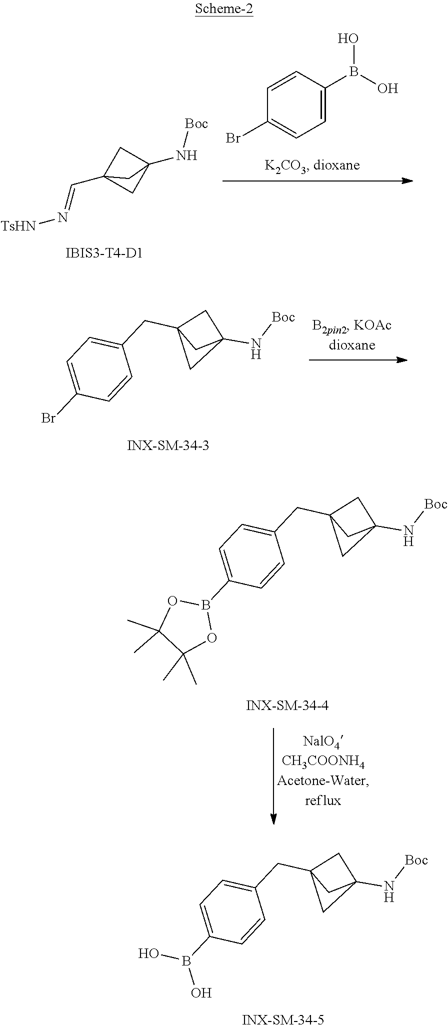

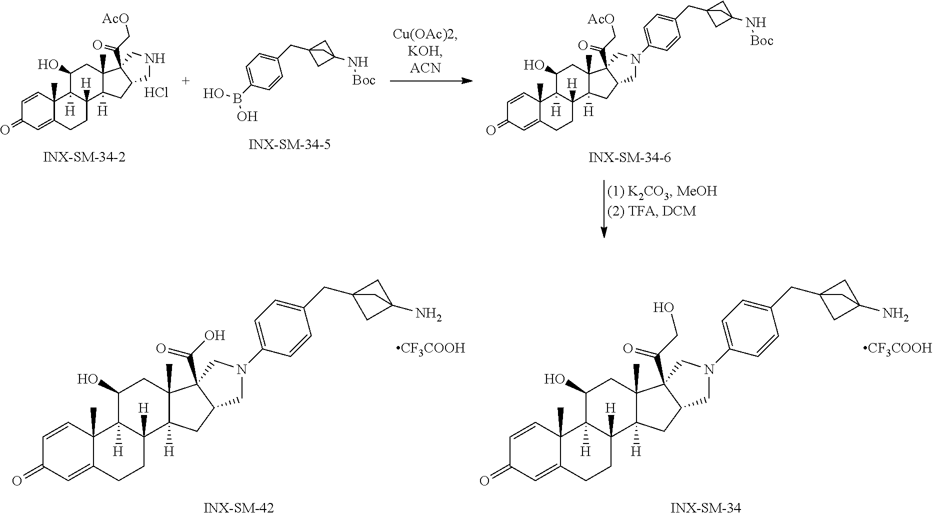

### Synthesis of 2-((6aR,6bS,7S,8aS,8bS,11aR,12aS,12bS)-10-benzyl-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-34-1)

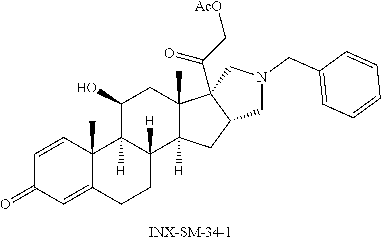

### Procedure

A 100 mL single-necked round bottom flask was charged with (110)-21-(Acetyloxy)-11-hydroxypregna-1,4, 16-triene-3,20-dione (5.0 g, 13.0 mmol) and 1, 4-dioxane (50 mL). To this solution, N-(Methoxymethyl)-N-(trimethylsilylmethyl)benzylamine (30.8 g, 13.0 mmol) and TFA (0.10 g, 0.9 mmol) were added and the reaction mixture was heated at 110° C. for 4 h. After completion of reaction as indicated by TLC, reaction mixture was concentrated. The crude was purified by silica gel column chromatography (ethyl acetate:hexane, 20:80) to give title compound as off white solid (3.0 g, 44.56%). LCMS 518.29 [M+H]⁺

### Synthesis of 2-((6aR,6bS,7S,8aS,8bS,11aR,12aS,12bS)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate hydrochloride (INX-SM-34-2)

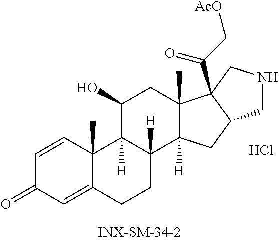

### Procedure

A 100 mL single-necked round bottom flask was charged with 2-((6aR,6bS,7S,8aS,8bS,11aR,12aS,12bS)-10-benzyl-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-34-1) (3.0 g, 5.79 mmol) and acetonitrile (20 mL). To this solution, NaHCO₃ (0.97 g, 11.5 mmol) and 1-chloroethyl chloroformate (1.65 g, 11.59) were added and stirred at room temperature for 2 h. After completion of reaction as indicated by TLC, reaction mixture was diluted with ethyl acetate and filtered through celite bed. The filtrate was concentrated under vacuum and the crude was triturated with n-pentene and diethyl ether to give title compound as white solid (2.0 g, 74.38%). LCMS 428.3 [M+H]⁺.

### Synthesis of tert-butyl (3-(4-bromobenzyl)bicyclo[1.1.1]pentan-1-yl)carbamate (INX-SM-34-3)

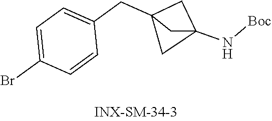 [SMILES: CC(C)(C)OC(=O)NC12CC(Cc3ccc(Br)cc3)(C1)C2]

### Procedure

A 10 mL glass vial was charged with tert-butyl (E)-(3-((2-tosylhydrazono)methyl) bicyclo[1.1.1]pentan-1-yl)carbamate (INX-SM-3-4) (0.05 g, 0.13 mmol) and 1, 4-dioxane (2 mL). To this solution, K₂CO₃ (0.039 g, 0.19 mmol) was added and N2 (g) was purged for 1 h. After precipitation observed in reaction mixture, (4-bromophenyl)boronic acid (0.039 g, 0.19 mmol) was added to the reaction and allowed to stir at 110° C. for 3 h. After completion of reaction as indicated by TLC, reaction mixture was diluted with ethyl acetate and washed with brine. The combined organic layer was dried over Na₂SO₄ and evaporated under vacuum. The crude was purified by silica gel column chromatography (ethyl acetate/hexane: 10:90) to give title compound as off white solid. (0.02 g, 43.0%). ¹H NMR (CDCl₃) δ: 7.39 (d, J=6.4 Hz, 2H)), 6.96 (d, J=6.4 Hz 2H), 2.79 (s, 2H), 1.81 (s, 6H) 1.44 (s, 9H).

### Synthesis of tert-butyl (3-(4-(4,4,5,5-tetramethyl-1,3,2-dioxaborolan-2-yl)benzyl)bicyclo[1.1.1]pentan-1-yl)carbamate (INX-SM-34-4)

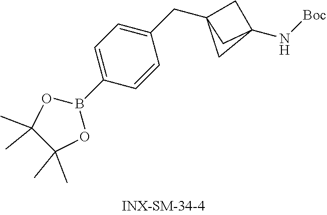 [SMILES: CC(C)(C)OC(=O)NC12CC(Cc3ccc(B4OC(C)(C)C(C)(C)O4)cc3)(C1)C2]

### Procedure

A 25 mL single necked round bottom flask was charged with tert-butyl (3-(4-bromobenzyl)bicyclo[1.1.1]pentan-1-yl)carbamate (INX-SM-34-3) (0.5 g, 1.42 mmol) and 1,4-dioxane (10 mL) under nitrogen. To this solution, bispinacolonediborane (1.07 g, 4.26 mmol) and potassium acetate (0.27 g, 2.84 mmol) were added purged with N₂ for 15 min. PdCl₂ (dppf)·DCM (0.12 g, 0.14 mmol) was added to the reaction mixture and heated at 110° C. for 2 h. After completion of reaction as indicated by TLC, reaction mixture was cooled at room temperature and diluted with ethyl acetate. It was filtered through celite bed and the combined organic layer was dried over Na₂SO₄ and evaporated under vacuum. The crude was purified by silica gel column chromatography (ethyl acetate/hexane: 20:80) to give title compound as off white semi solid (0.8 g, crude). It was used for next step.

### Synthesis of (4-((3-((tert-butoxycarbonyl)amino)bicyclo[1.1.1]pentan-1-yl)methyl) phenyl)boronic acid (INX-SM-34-5)

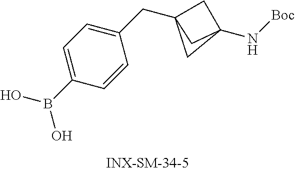 [SMILES: CC(C)(C)OC(=O)NC12CC(Cc3ccc(B(O)O)cc3)(C1)C2]

### Procedure

A 25 mL single-necked round bottom flask was charged with tert-butyl (3-(4-(4,4,5,5-tetramethyl-1,3,2-dioxaborolan-2-yl)benzyl)bicyclo[1.1.1]pentan-1-yl)carbamate INX-SM-34-4) (1.0 g, 2.5 mmol) and acetone: water (9:1) (10 mL). To this solution, NaIO₄ (4.28 g, 20.0 mmol) and ammonium acetate (1.54 g, 20.0 mmol) were added and the reaction mixture was allowed to stir at reflux temperature for 2 h. After completion of reaction as indicated by TLC, reaction mixture was cooled to room temperature, diluted with ethyl acetate and filtered through celite bed. The combined organic layer was dried over Na₂SO₄ and evaporated under vacuum. The crude was triturated with n-pentene and diethyl ether to give title compound as off white solid (0.4 g, 50.3%). LCMS: 262.2 [M+1-t-Bu]

### Synthesis of 2-((6aR,6bS,7S,8aS,8bS,11aR,12aS,12bS)-10-(4-((3-((tert-butoxycarbonyl)amino)bicyclo[1.1.1]pentan-1-yl)methyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-34-6)

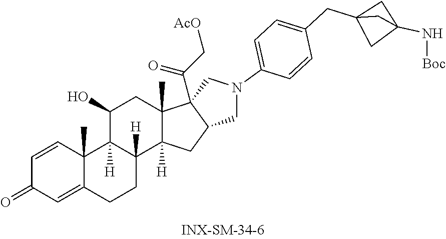

### Procedure

A 35 mL glass vial was charged with 2-((6aR,6bS,7S,8aS,8bS,11aR,12aS,12bS)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate hydrochloride INX-SM-34-2) (0.1 g, 0.23 mmol) and (4-((3-((tert-butoxycarbonyl)amino) bicyclo[1.1.1]pentan-1-yl)methyl)phenyl)boronic acid INX-SM-34-5) (0.25 g, 0.81 mmol) in acetonitrile (5 mL). To this solution, KOH (0.13 g, 2.34 mmol) and Cu (OAc) 2 (0.12 g, 0.70 mmol) were added and stirred the reaction mixture at room temperature for 2 h. After completion of reaction as indicated by TLC, the reaction mixture was diluted with ethyl acetate and filtered through celite bed. The combined organic layer was dried over Na₂SO₄ and evaporated under vacuum. The crude was purified by silica gel column chromatography (ethyl acetate/hexane: 30:70) to give title compound as off white solid (0.15 g, 93.31%). LCMS: 699.5 [M+H]⁺.

### Synthesis of (6aR,6bS,7S,8aS,8bS,11aR,12aS,12bS)-10-(4-((3-aminobicyclo[1.1.1]pentan-1-yl)methyl)phenyl)-7-hydroxy-8b-(2-hydroxyacetyl)-6a,8a-dimethyl-1,6a,6b,7,8,8a,8b,9,10,11,11a,12,12a,12b-tetradecahydronaphtho[2′,1′:4,5]indeno[1,2-c]pyrrol-4 (2H)-one 2,2,2-trifluoroacetate (INX-SM-34); and

(6aR,6bS,7S,8aS,8bS,11aR,12aS,12bS)-10-(4-((3-aminobicyclo[1.1.1]pentan-1-yl)methyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrole-8b(2H)-carboxylic acid compound with 2,2,2-trifluoroacetic acid (INX-SM-42)

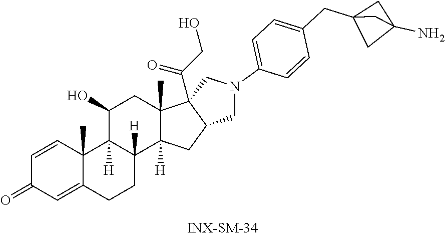

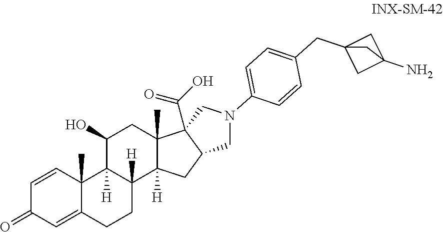

### Procedure

A 25 mL single-necked round bottom flask was charged with 2-((6aR,6bS,7S,8aS,8bS,11aR,12aS,12bS)-10-(4-((3-((tert-butoxycarbonyl)amino)bicyclo[1.1.1]pentan-1-yl)methyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-34-6) (0.13 g, 0.18 mmol) and methanol (2 mL). To this solution, K2CO3 (0.038 g, 0.27 mmol) was added and allowed to stir at room temperature for 1 h. After completion of reaction as indicated by TLC, reaction mixture was diluted with ethyl acetate and filtered through celite bed. The combine organic layer was evaporated under vacuum and dissolved in DCM (2 mL). To this solution, TFA (0.2 ml) was added and stirred for 2 h at room temperature. After completion of reaction as indicated by TLC, reaction mixture was evaporated under vacuum. The crude was purified by prep-HPLC (Column: YMC-Actus Triart Prep C18-S, 250×20 mm S-5 μm, 12 nm, Mobile phase: A=0.05% TFA in water, B=MTBE: acetonitrile (90:10), A:B=78:22) to give INX-SM-34 (0.010 g, 8.01%). LCMS: 557.30 [M+H]⁺ ¹H NMR (400 MHZ, MeOD: δ: 7.49 (d, J=10.4 Hz, 1H), 6.94 (d, J=8.8 Hz, 2H), 6.62 (d, J=8.4 Hz, 2H), 6.26 (dd, J=10.0 & 2.0 Hz, 1H), 5.99 (s, 1H), 4.60-4.25 (m, 3H), 3.60-3.00 (m, 4H), 2.77-1.55 (m, 18H), 1.50 (s, 3H), 1.15 (s, 3H), 1.15-1.00 (m, 2H). and INX-SM-42 (0.0041 g, 3.36%). LCMS: 543.3 [M+H]⁺; ¹H NMR (400 MHZ, MeOD: δ: 7.50 (d, J=10.4 Hz, 1H), 6.94 (d, J=8.4 Hz, 2H), 6.60 (d, J=8.4 Hz, 2H), 6.26 (dd, J=10.0& 2.0 Hz, 1H), 5.99 (s, 1H), 4.45 (br s, 1H), 3.60-3.30 (m, 4H), 2.77 (s, 2H), 2.70-1.55 (m, 16H), 1.50 (s, 3H), 1.22 (s, 3H), 1.15-1.00 (m, 2H).

### Synthesis of (S)-4-(2-(2-bromoacetamido)acetamido)-5-((3-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)bicyclo[1.1.1]pentan-1-yl)amino)-5-oxopentanoic acid (INX-A13)

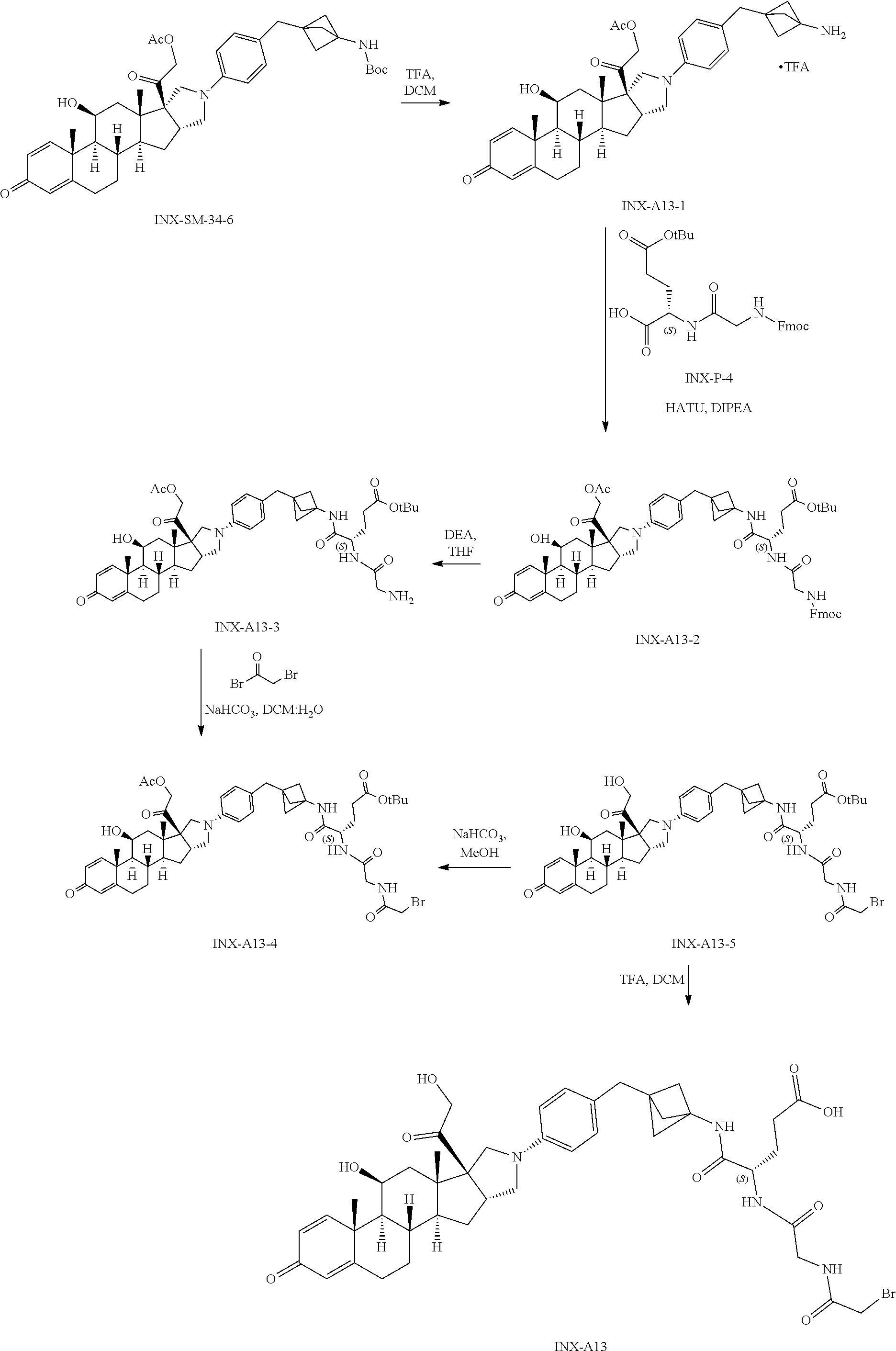

### Synthesis of 2-((6aR,6bS,7S,8aS,8bS,11aR,12aS,12bS)-10-(4-((3-aminobicyclo[1.1.1]pentan-1-yl) methyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate 2,2,2-trifluoroacetate (INX-A13-1)

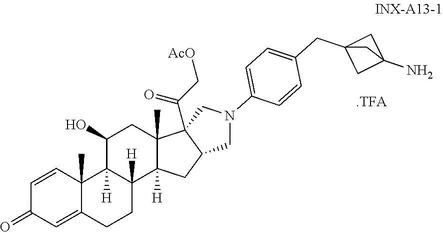

### Procedure

A 10 mL single-necked round bottom flask was charged with 2-((6aR,6bS,7S,8aS,8bS,11aR,12aS,12bS)-10-(4-((3-((tert-butoxycarbonyl)amino)bicyclo[1.1.1]pentan-1-yl)methyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-34-6) (0.33 g, 0.47 mmol) and DCM (5 mL). To this solution, TFA (0.1 mL) was added and stirred for 2 h at room temperature. After completion of reaction as indicated by TLC, reaction mixture was evaporated under vacuum. The crude was triturated with diethyl ether to give title compound as off white solid (0.28 g, quantitative). LCMS: 599.5 [M+H]⁺

### Synthesis of tert-butyl(S)-4-(2-((((9H-fluoren-9-yl)methoxy)carbonyl)amino)acetamido)-5-((3-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)bicyclo[1.1.1]pentan-1-yl)amino)-5-oxopentanoate (INX-A13-2)

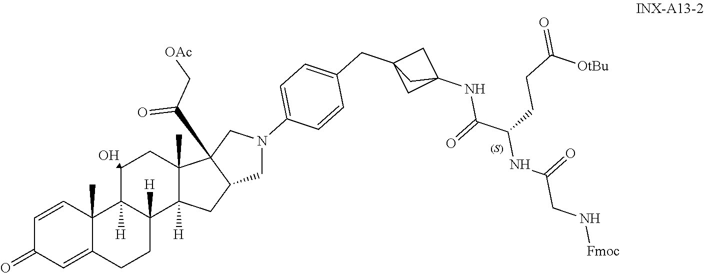

### Procedure

A 10 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) (0.24 g, 0.50 mmol), 2-((6aR,6bS,7S,8aS,8bS,11aR,12aS,12bS)-10-(4-((3-aminobicyclo[1.1.1]pentan-1-yl)methyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate 2,2,2-trifluoroacetate (INX-A13-1) (0.3 g, 0.50 mmol) and DMF (2 mL). To this solution DIPEA (0.16 g, 1.25 mmol) and HATU (0.22 g, 0.60 mmol) were added and stirred for 15 min at room temperature. After completion of reaction as indicated by TLC, reaction mixture was poured into water and extracted with ethyl acetate. The combined organic layer was dried over Na₂SO₄ and evaporated under vacuum. The crude was triturated with diethyl ether to give title compound as off white solid (0.4 g, 74.99%). LCMS: 1063.7 [M+H]⁺

### Synthesis of tert-butyl(S)-5-((3-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)bicyclo[1.1.1]pentan-1-yl)amino)-4-(2-aminoacetamido)-5-oxopentanoate (INX-A13-3)

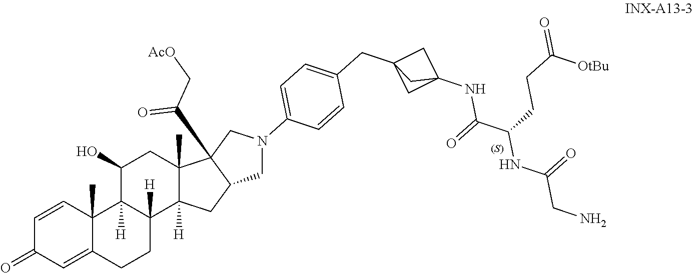

### Procedure

A 10 mL single-necked round bottom flash was charged with tert-butyl(S)-4-(2-((((9H-fluoren-9-yl)methoxy)carbonyl)amino)acetamido)-5-((3-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)bicyclo[1.1.1]pentan-1-yl)amino)-5-oxopentanoate (INX-A13-2) (0.4 g, 0.37 mmol) and THF (5 mL). To this solution, diethyl amine (0.55 g, 7.5 mmol) was added and stirred for 2 h. After completion of reaction as indicated by TLC, reaction mixture was evaporated under vacuum and triturated with diethyl ether and pentane to give title compound as yellow solid (0.22 g, Crude). It was used for next step without analysis.

### Synthesis of tert-butyl(S)-5-((3-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12, 12a,12b-tetradecahydronaphtho[2′,1′:4,5]indeno[1,2-c]pyrrol-10(2H)-yl)benzyl) bicyclo[1.1.1]pentan-1-yl)amino)-4-(2-(2-bromoacetamido)acetamido)-5-oxopentanoate (INX-A13-4)

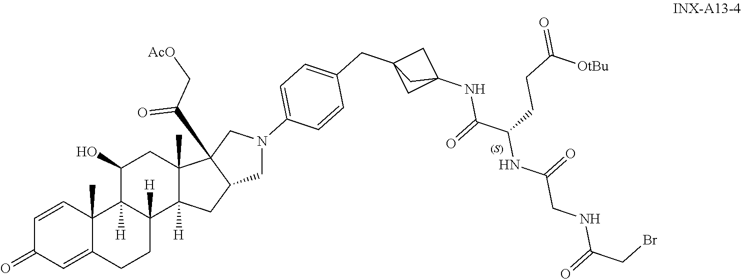

A 10 mL single-necked round bottom flash was charged with tert-butyl(S)-5-((3-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)bicyclo[1.1.1]pentan-1-yl)amino)-4-(2-aminoacetamido)-5-oxopentanoate (INX-A13-3) (0.22 g, 0.26 mmol) and DCM (4 mL). To this solution, NaHCO₃ (0.06 g, 0.78 mmol) solution in water (1 mL) and bromoacetyl bromide (0.04 g, 0.20 mmol) were added dropwise and stirred for 1 h at room temperature. After completion of reaction as indicated by TLC, reaction mixture was quenched with water and extracted with DCM. The combined organic layer was dried over Na₂SO₄ and evaporated under vacuum to give title compound as off white solid (0.24 g, Crude). It was taken to the next step without purification. LCMS: 963.5 [M+H]⁺.

### Synthesis of tert-butyl (S)-4-(2-(2-bromoacetamido)acetamido)-5-((3-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)bicyclo[1.1.1]pentan-1-yl)amino)-5-oxopentanoate (INX-A13-5)

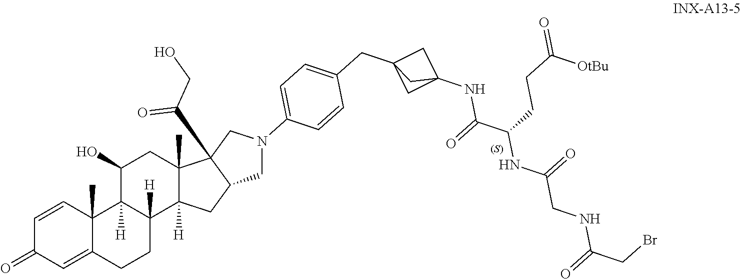

### Procedure

A 10 mL single-necked round bottom flask charged with tert-butyl(S)-5-((3-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)bicyclo[1.1.1]pentan-1-yl)amino)-4-(2-(2-bromoacetamido)acetamido)-5-oxopentanoate (INX-A13-4) (0.12 g, 0.12 mmol) and Methanol: H₂O (9:1, 5 mL). To this solution, NaHCO₃ (0.020 g, 0.24 mmol) was added and stirred the reaction mixture for 6 h at room temperature. After completion of reaction as indicated by TLC, reaction mixture was diluted with ethyl acetate and filtered through celite bed. The combine organic layer was evaporated under vacuum to give title compound as off white solid (0.08 g, 72.46%). LCMS: 920.5 [M+H]⁺.

### Synthesis of (S)-4-(2-(2-bromoacetamido)acetamido)-5-((3-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)bicyclo[1.1.1]pentan-1-yl)amino)-5-oxopentanoic acid (INX-A13)

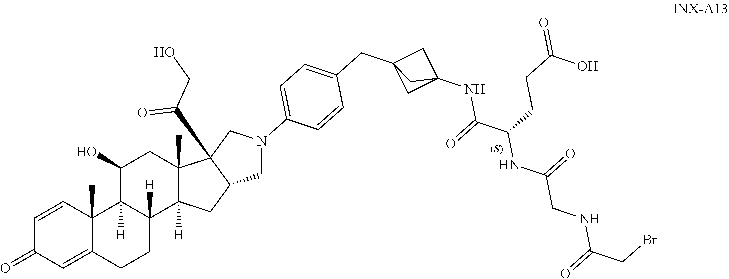

### Procedure

A 10 mL single-necked round bottom flash was charged with tert-butyl(S)-4-(2-(2-bromoacetamido)acetamido)-5-((3-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)bicyclo[1.1.1]pentan-1-yl)amino)-5-oxopentanoate (INX-A13-5) (0.08 g, 0.08 mmol) and DCM (5 mL). To this solution, TFA (0.4 ml) was added and stirred for 2 h at room temperature. After completion of reaction as indicated by TLC, reaction mixture was evaporated under vacuum The crude was purified by prep HPLC (SUNFIRE Prep C18 OBD, 19×250 mm, 5 μm, Mobile phase: A=0.1% FA in water, B=Acetonitrile, A:B=58:42, retention time 13 min) to give title compound as off white solid (0.007 g, 10.12%). LCMS: 863.4 [M+H]⁺; ¹H NMR (400 MHZ, MeOD): 0 7.48 (d, J=10 Hz, 1H), 6.95-6.80 (m, 3H), 6.60 (d, J=8.8 Hz, 1H), 6.26 (d, 1H), 5.99 (m, 1H), 4.51-3.50 (m, 8H), 3.51-2.60 (m, 4H), 2.50-2.00 (s, 8H), 2.00-1.20 (m, 22H).

### Synthesis of (6aR,6bS,7S,8aS,8bS,11aR,12aS,12bS)-10-(4-((6-aminospiro[3.3]heptan-2-yl)methyl)phenyl)-7-hydroxy-8b-(2-hydroxyacetyl)-6a,8a-dimethyl-1,6a,6b,7,8,8a,8b,9,10,11,11a,12,12a,12b-tetradecahydronaphtho[2′,1′:4,5]indeno[1,2-c]pyrrol-4 (2H)-one (INX-SM-37)

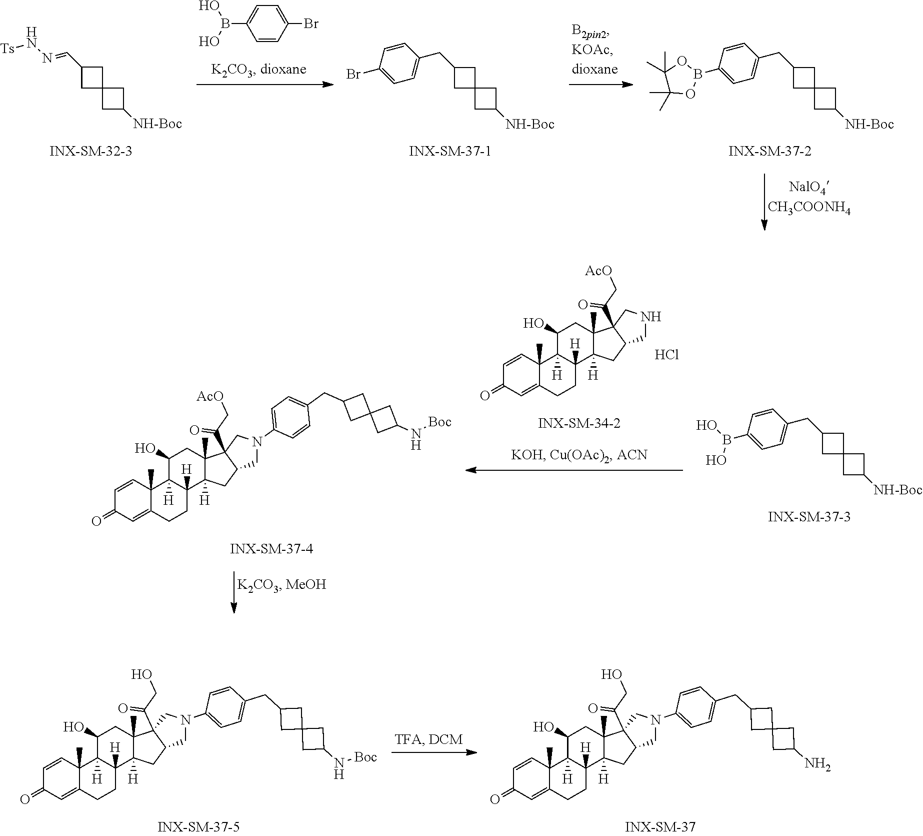

### Synthesis of tert-butyl (6-(4-bromobenzyl) spiro[3.3]heptan-2-yl)carbamate (INX-SM-37-1)

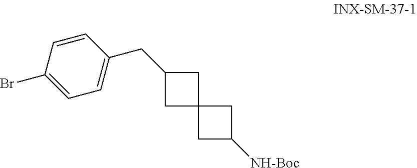

### Procedure

A 100 ml single-necked round bottom flash was charged with tert-butyl (E)-(6-((2-tosylhydrazono)methyl)spiro[3.3]heptan-2-yl)carbamate (INX-SM-32-3) (2.0 g, 0.49 mmol) and Dioxane (80 mL). To this solution, (4-bromophenyl)boronic acid (1.47 g, 0.73 mmol) and K2CO3 (1.0 g, 0.73 mmol) were added at room temperature and stirred for 2 h at 100° C. After completion of reaction as indicated by TLC, reaction mixture was quenched with sat. NaHCO₃ solution and extracted with ethyl acetate. The combined organic layer was dried over Na₂SO₄ and evaporated under vacuum. The crude was purified by silica gel column chromatography (ethyl acetate: Hexane, 5:95) to give title compound as white solid (0.4 g, 21.43%). LCMS: 380 [M+H]⁺.

Step-5: tert-butyl (6-(4-(4,4,5,5-tetramethyl-1,3,2-dioxaborolan-2-yl)benzyl) spiro[3.3]heptan-2-yl)carbamate (INX-SM-37-2)

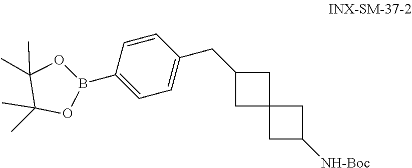

### Procedure

A 35 mL glass vial was charged with tert-butyl (6-(4-bromobenzyl) spiro[3.3]heptan-2-yl)carbamate (INX-SM-37-1) (0.2 g, 0.5 mmol) and dioxane (6 mL). To this solution, bis (pinacolato)diboron (0.39 g, 0.15 mmol) and KOAc (0.1 g, 0.10 mmol) were added and purged with N₂ for 30 min. To this solution, Pd (dppf) Cl₂·CH₂Cl₂ (0.04 g, 0.05 mmol) was added and stirred at 100° C. for 1 h. After completion of reaction as indicated by TLC, reaction mixture was quenched with sat. NaHCO₃ solution and extracted with ethyl acetate. The combined organic layer was dried over Na₂SO₄ and evaporated under vacuum. The crude was purified by silica gel column chromatography (ethyl acetate:hexane, 5:95) to give title compound as white solid (0.3 g, quantitative). LCMS: 327.2 [[M+H-Boc]⁺].

### Synthesis of (4-((6-((tert-butoxycarbonyl)amino) spiro[3.3]heptan-2-yl)methyl)phenyl) boronic acid (INX-SM-37-3)

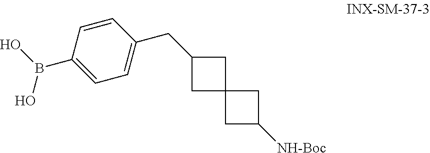

### Procedure

A 25 ml single-necked round bottom flash was charged with tert-butyl (6-(4-(4,4,5,5-tetramethyl-1,3,2-dioxaborolan-2-yl)benzyl) spiro[3.3]heptan-2-yl)carbamate (INX-SM-37-2) (IBIS-T-66-A2) (0.3 g, 0.70 mmol) and acetone: water (9:1, 3 mL). To this solution, NaIO₄ (1.2 g, 5.6 mmol) and CH₃COONH₄ (0.43 g, 0.56 mmol) were added at room temperature and stirred at 50° C. for 3 h. After completion of reaction as indicated by TLC, reaction mixture was quenched with water and extracted with ethyl acetate. The combined organic layer was dried over Na₂SO₄ and evaporated under vacuum to give title compound as off white solid (0.18 g, 74.87%). LCMS: 245.7 [[M+H−Boc]⁺].

### Synthesis of 2-((6aR,6bS,7S,8aS,8bS,11aR,12aS,12bS)-10-(4-((6-((tert-butoxycarbonyl)amino) spiro[3.3]heptan-2-yl)methyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-37-4)

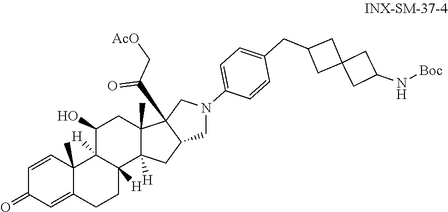 [SMILES: CC(=O)OCC(=O)[C@@]12CN(c3ccc(CC4CC5(C4)CC(NC(=O)OC(C)(C)C)C5)cc3)C[C@@H]1C[C@H]1[C@@H]3CCC4=CC(=O)C=C[C@]4(C)[C@H]3[C@@H](O)C[C@@]12C]

### Procedure

A 35 mL glass vial was charged with 2-((6aR,6bS,7S,8aS,8bS,11aR,12aS,12bS)-7-hydroxy-6a,8a-dimethyl-4-oxo-1,4,6a,6b,7,8,8a,9, 10, 11, 11a,12,12a,12b-tetradecahydronaphtho[2′,1′:4,5]indeno[1,2-c]pyrrol-8b(2H)-yl)-2-oxoethyl acetate hydrochloride (INX-SM-34-2) (0.12 g, 0.28 mmol), (4-((6-((tert-butoxycarbonyl) amino) spiro[3.3]heptan-2-yl)methyl)phenyl)boronic acid (INX-SM-37-3) (0.29 g, 0.84 mmol) and acetonitrile (12 mL). To this solution, KOH (0.15 g, 2.80 mmol) and Cu (OAc) 2 (0.15 g, 0.84 mmol) were added and stirred the reaction mixture at room temperature for 2 h. After completion of reaction as indicated by TLC, the reaction mixture was diluted with ethyl acetate and filtered through celite. The collected organic layer was dried over Na₂SO₄ and evaporated under vacuum. The crude was purified by silica gel column chromatography (ethyl acetate/hexane, 50:50) to give title compound as off white solid (0.060 g, 31.91%). LCMS: 727.6 [M+H]⁺.

### Synthesis of tert-butyl (6-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl) spiro[3.3]heptan-2-yl)carbamate (INX-SM-37-5)

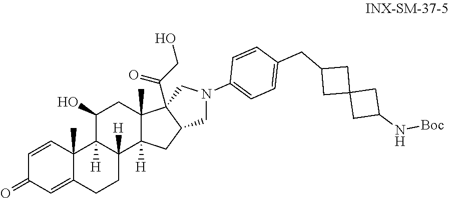

### Procedure

A 10 mL single-necked round bottom flask was charged with 2-((6aR,6bS,7S,8aS,8bS,11aR,12aS,12bS)-10-(4-((6-((tert-butoxycarbonyl)amino) spiro[3.3]heptan-2-yl)methyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-37-4) (0.06 g, 0.082 mmol) and methanol (1 mL). To this solution, NaHCO₃ (0.013 g, 0.16 mmol) was added and stirred at room temperature for 16 h. After completion of reaction as indicated by TLC, reaction mixture was concentrated to give title compound as off white solid (0.060 g, quantitative). LCMS: 585.4 [[M+H]⁺-Boc].

### Synthesis of (6aR,6bS,7S,8aS,8bS,11aR,12aS,12bS)-10-(4-((6-aminospiro[3.3]heptan-2-yl)methyl)phenyl)-7-hydroxy-8b-(2-hydroxyacetyl)-6a,8a-dimethyl-1,6a,6b,7,8,8a,8b,9,10,11,11a,12,12a,12b-tetradecahydronaphtho[2′,1′:4,5]indeno[1,2-c]pyrrol-4 (2H)-one (INX-SM-37)

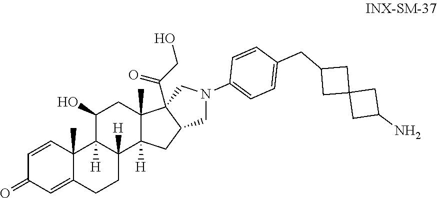

### Procedure

A 10 mL single-necked round bottom flask was charged with tert-butyl (6-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl) spiro[3.3]heptan-2-yl)carbamate (INX-SM-37-5) (0.050 g) and DCM (2 mL). To this solution, TFA (0.5 ml) was added and stirred for 2 h at room temperature. After completion of reaction as indicated by TLC, reaction mixture was evaporated under vacuum. The crude was purified by prep-HPLC (Column: YMC-Actus Triart Prep C18-S, 250×20 mm S-5 μm, 12 nm, Mobile phase: A=0.1% FA in water, B=Acetonitrile; retention time 10.27 min) to give title compound as off white solid (0.011 g, 23.42%). LCMS: 585.4 [M+H]⁺; ¹H NMR (400 MHZ, MeOD) δ: 7.48 (d, J=10 Hz, 1H), 6.95-6.83 (m, 3H), 6.58 (d, J=8.8 Hz, 1H), 6.26 (d, 10.0 Hz, 1H), 5.98 (s, 1H), 4.50-3.00 (m, 8H), 2.60-1.55 (m, 21H), 1.50 (s, 3H), 1.20-1.05 (m, 2H), 1.06 (s, 3H).

### Synthesis of (S)-4-(2-(2-bromoacetamido)acetamido)-5-((6-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11, 11a,12,12a,12b-tetradecahydronaphtho[2′,1′:4,5]indeno[1,2-c]pyrrol-10(2H)-yl)benzyl) spiro[3.3]heptan-2-yl)amino)-5-oxopentanoic acid (INX-A10)

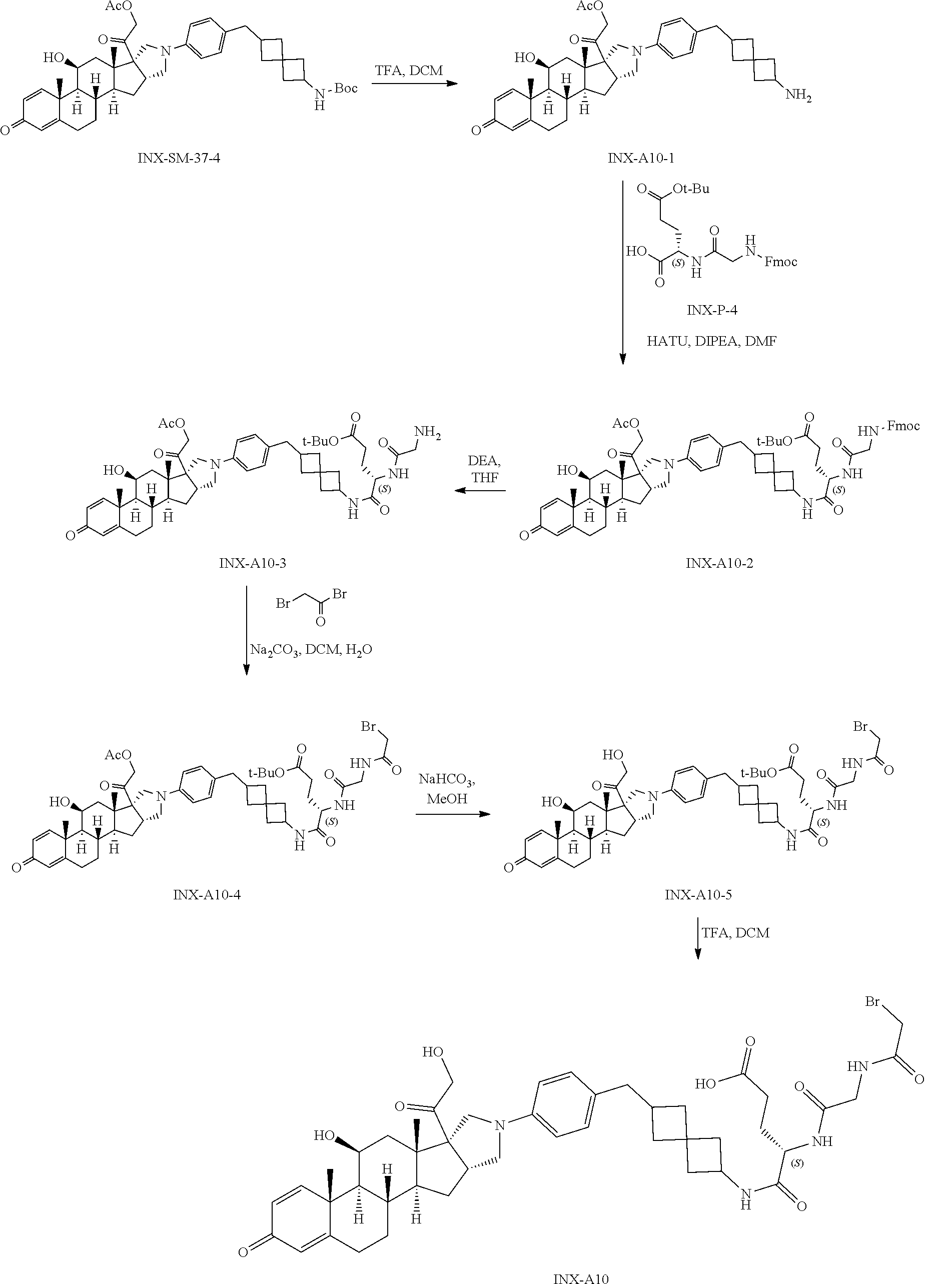

### Synthesis of 2-((6aR,6bS,7S,8aS,8bS,11aR,12aS,12bS)-10-(4-((6-aminospiro[3.3]heptan-2-yl)methyl)phenyl)-7-hydroxy-6a,8a-dimethyl-4-oxo-1,4,6a,6b,7,8,8a,9,10,11,11a,12,12a,12b-tetradecahydronaphtho[2,1′: 4,5]indeno[1,2-c]pyrrol-8b(2H)-yl)-2-oxoethyl acetate (INX-A10-1)

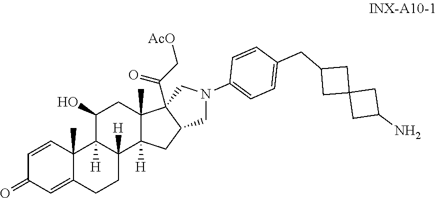

A 25 mL single-necked round bottom flask was charged with 2-((6aR,6bS,7S,8aS,8bS,11aR,12aS,12bS)-10-(4-((6-((tert-butoxycarbonyl)amino) spiro[3.3]heptan-2-yl)methyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-37-4) (0.3 g) and DCM (5 mL). To this solution, TFA (1 mL) was added and stirred for 1 h at room temperature. After completion of reaction as indicated by TLC, reaction mixture was evaporated under vacuum and the crude was purified by trituration with diethyl ether to give title compound as off white solid (0.30 g, quantitative). LCMS: 627.41 [M+H]⁺

### Synthesis of tert-butyl(S)-4-(2-((((9H-fluoren-9-yl)methoxy)carbonyl)amino)acetamido)-5-((6-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl) spiro[3.3]heptan-2-yl)amino)-5-oxopentanoate (INX-A10-2)

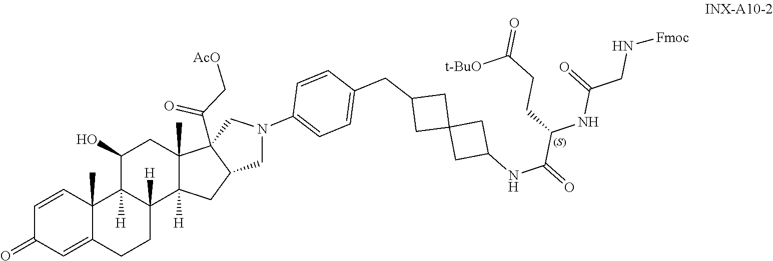

### Procedure

A 25 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) (0.230 g, 0.47 mmol), 2-((6aR,6bS,7S,8aS,8bS,11aR,12aS,12bS)-10-(4-((6-aminospiro[3.3]heptan-2-yl)methyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-A10-1) (0.3 g, 0.47 mmol) and DMF (6 mL). To this solution, DIPEA (0.185 g, 1.25 mmol) and HATU (0.27 g, 0.71 mmol) were added and stirred for 15 min at room temperature. After completion of reaction as indicated by TLC, reaction mixture was poured into water and the solid was filtered and dried under vacuum. The crude was purified by silica gel column chromatography (water/acetonitrile, 13:87) to give title compound as white solid (0.380 g, 74.0%). LCMS: 1091.7 [M+H]⁺.

### Synthesis of tert-butyl(S)-5-((6-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl) spiro[3.3]heptan-2-yl)amino)-4-(2-aminoacetamido)-5-oxopentanoate (INX-A10-3)

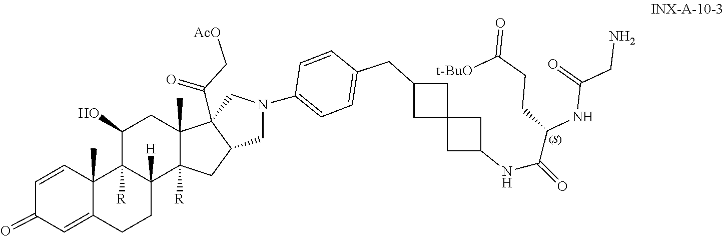

### Procedure

A 25 mL single-necked round bottom flash was charged with tert-butyl(S)-4-(2-((((9H-fluoren-9-yl)methoxy)carbonyl)amino)acetamido)-5-((6-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl) spiro[3.3]heptan-2-yl)amino)-5-oxopentanoate (INX-A10-2) (0.3 g, 0.27 mmol) and THF (54 mL). To this solution, diethyl amine (0.20 g, 2.7 mmol) was added at room temperature and stirred for 2 h. After completion of reaction as indicated by TLC, reaction mixture was evaporated under vacuum and triturated with diethyl ether-pentane to give title compound as white solid (0.22 g, 93.75%). LCMS: 869.7 [M+H]⁺.

### Synthesis of tert-butyl(S)-5-((6-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl) spiro[3.3]heptan-2-yl)amino)-4-(2-(2-bromoacetamido)acetamido)-5-oxopentanoate (INX-A10-4)

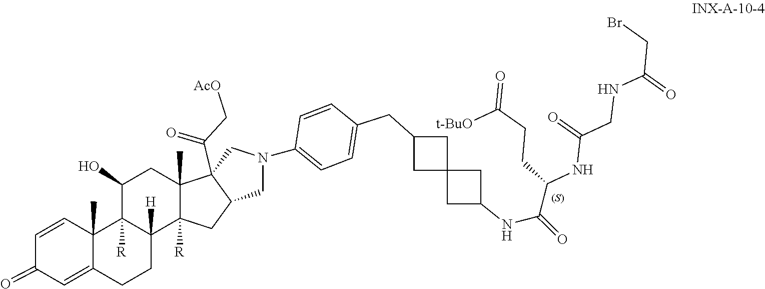

### Procedure

A 25 mL single-necked round bottom flash was charged with tert-butyl(S)-5-((6-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl) spiro[3.3]heptan-2-yl)amino)-4-(2-aminoacetamido)-5-oxopentanoate (INX-A10-3) (0.22 g, 0.25 mmol) and DCM (3 mL). To this solution, NaHCO₃ (0.053 g, 0.50 mmol) dissolved in water (1 mL) and bromoacetyl bromide (0.076 g, 0.37 mmol) were added at room temperature and stirred for 30 min. After completion of reaction as indicated by TLC, reaction mixture was quenched with water and extracted with DCM. The combined organic layer was dried over Na₂SO₄ and evaporated under vacuum to give title compound as off white solid (0.21 g, 84.8%). LCMS: 989.6 [M+H]⁺.

### Synthesis of tert-butyl(S)-4-(2-(2-bromoacetamido)acetamido)-5-((6-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl) spiro[3.3]heptan-2-yl)amino)-5-oxopentanoate (INX-A10-5)

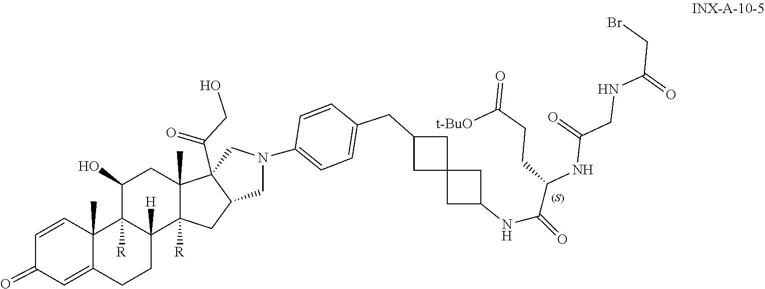

### Procedure

A 10 mL single-necked round bottom flask was charged with tert-butyl(S)-5-((6-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl) spiro[3.3]heptan-2-yl)amino)-4-(2-(2-bromoacetamido)acetamido)-5-oxopentanoate (INX-A10-4) (0.210 g, 0.21 mmol) and methanol (2 mL). To this solution, NaHCO₃ (0.035 g, 0.42 mmol) was added and stirred the reaction mixture at room temperature for 16 h. After completion of reaction as indicated by TLC, reaction mixture was diluted with ethyl acetate and filtered through celite bed. The combine organic layer was concentrated to give title compound as off white solid (0.220 g, quantitative). LCMS: 947.3 [M+H]⁺.

### Synthesis of (S)-4-(2-(2-bromoacetamido)acetamido)-5-((6-(4-((6aR,6bS,7S,8aS,8bS,11aR, dimethyl-4-oxo-1,4,6a,6b,7,8,8a,8b,9,11, 12aS,12bS)-7-hydroxy-8b-(2-hydroxyacetyl)-6a,8a-11a,12,12a,12b-tetradecahydronaphtho[2′,1′:4,5]indeno[1,2-c]pyrrol-10(2H)-yl)benzyl) spiro[3.3]heptan-2-yl)amino)-5-oxopentanoic acid (INX-A10)

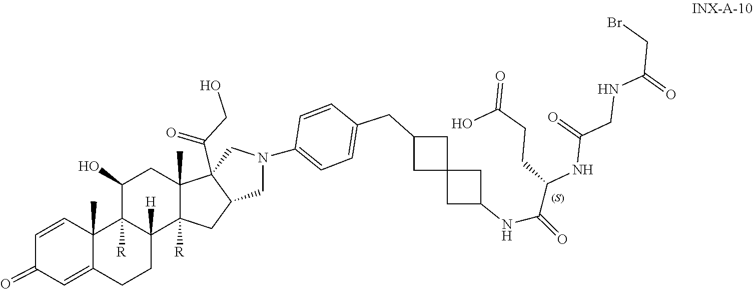

A 10 mL single-necked round bottom flash was charged with tert-butyl(S)-4-(2-(2-bromoacetamido)acetamido)-5-((6-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl) spiro[3.3]heptan-2-yl)amino)-5-oxopentanoate INX-A10-5) (0.220 g, 0.232 mmol) and DCM (3 mL). To this solution, TFA (1.5 ml) was added and stirred the reaction mixture for 1 h at room temperature. After completion of reaction as indicated by TLC, reaction mixture was evaporated under vacuum. The crude was purified by prep HPLC (SUNFIRE Prep C18 OBD (250×19) mm, 5 μm, Mobile phase: A=0.05% TFA in water, B=Acetonitrile, A:B=55:45, retention time: 15.67 min) to give title compound as white solid (0.026 g, 12.56%). LCMS: 891.5 [M+H]⁺; ¹H NMR (400 MHZ, MeOD: δ: 7.48 (d, J=10 Hz, 1H), 6.95 (d, J=8.4 Hz, 2H), 6.62 (d, J=8.4 Hz, 2H), 6.26 (dd, J=10.0&2.0 Hz, 1H), 5.99 (s, 1H), 4.50-4.27 (m, 4H), 4.20-3.80 (m, 5H), 3.60-2.90 (m, 4H), 2.50-1.55 (m, 25H), 1.50 (s, 3H), 1.20-1.00 (m, 2H), 1.07 (s, 3H).

### Synthesis of (2S,6aS,6bR,7S,8aS,8bS,11aR,12aS,12bS)-10-(4-(3-aminobenzyl)phenyl)-2,6b-difluoro-7-hydroxy-8b-(2-hydroxyacetyl)-6a,8a-dimethyl-1,6a,6b,7,8,8a,8b,9,10,11,11a,12,12a,12b-tetradecahydronaphtho[2′,1′:4,5]indeno[1,2-c]pyrrol-4 (2H)-one (INX-SM-28)

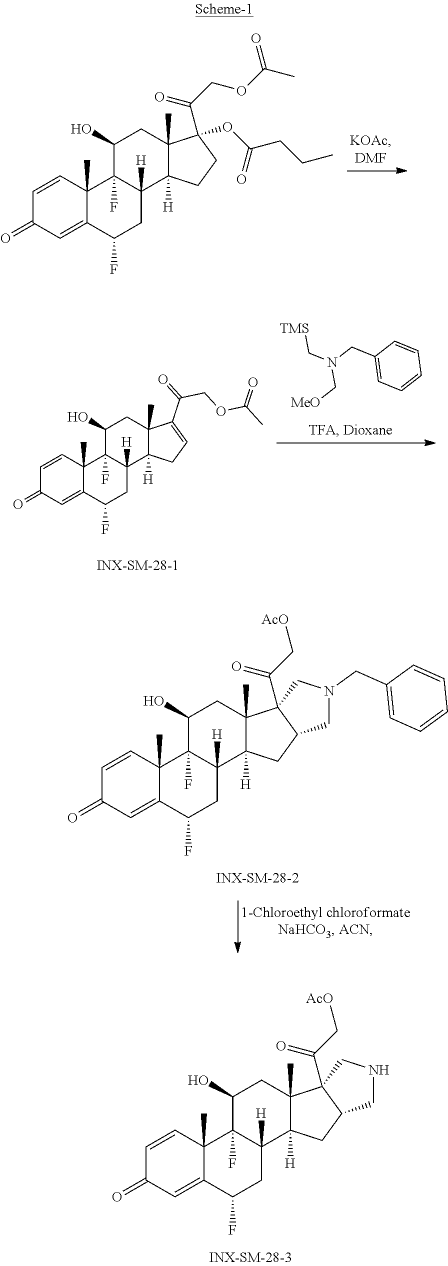

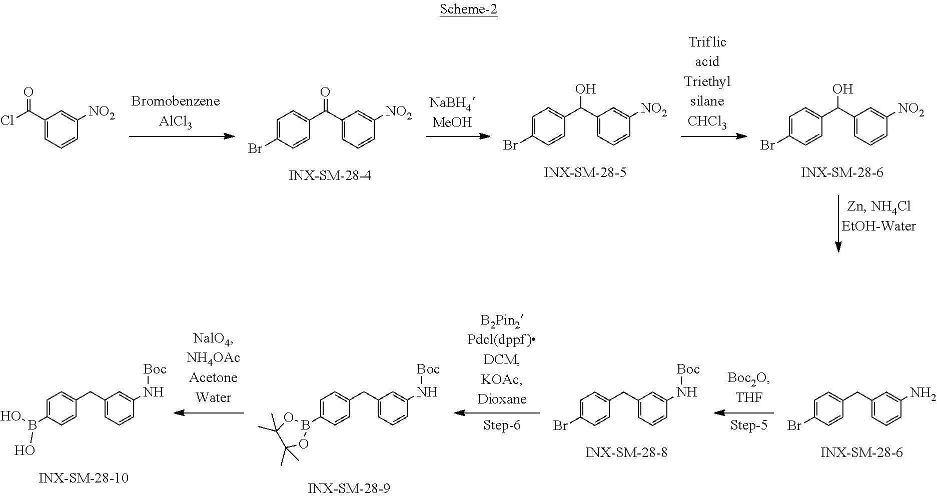

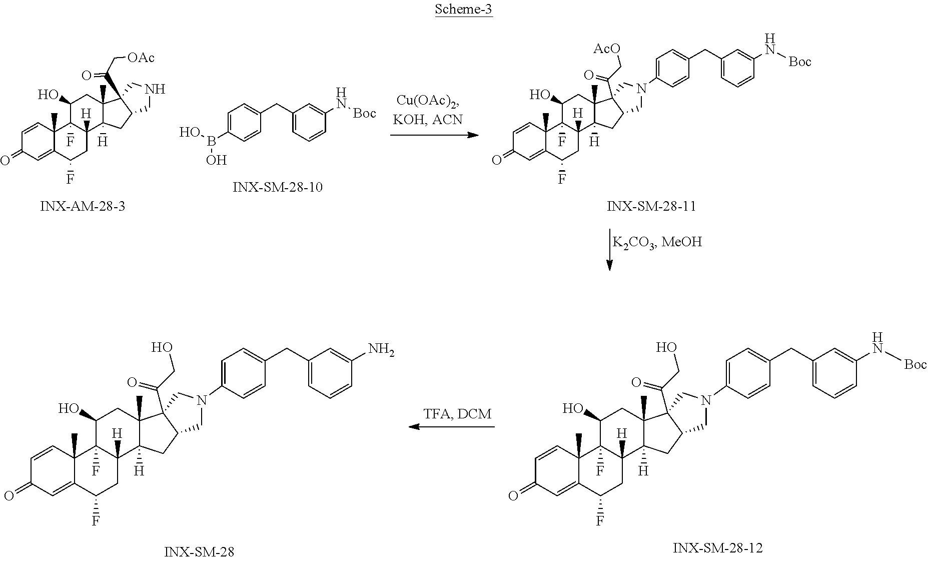

### Synthesis of 2-((6S,8S,9R,10S,11S,13S,14S)-6,9-difluoro-11-hydroxy-10,13-dimethyl-3-oxo-6,7,8,9,10,11,12,13,14,15-decahydro-3H-cyclopenta[a]phenanthren-17-yl)-2-oxoethyl acetate (INX-SM-28-1)

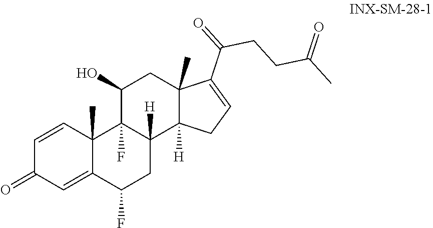

### Procedure

A 100 mL sealed tube was charged with Difluprednate (5.0 g, 9.84 mmol) and DMF (50 mL). To this solution, potassium acetate (7.71 g, 7.87 mmol) was added and stirred at 100° C. for 16 h. After completion of reaction as indicated by TLC, reaction mixture was poured into water and solid was filtered to obtain title compound as white solid (4.0 g, 96.76%). LC/MS: 421.3 [M+H]⁺.

### Synthesis of 2-((2S,6aS,6bR,7S,8aS,8bS,11aR,12aS,12bS)-10-benzyl-2,6b-difluoro-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-28-2)

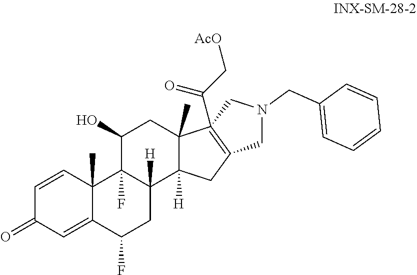

A 100 mL glass seal tube was charged with 2-((6S,8S,9R,10S,11S,13S,14S)-6,9-difluoro-11-hydroxy-10, 13-dimethyl-3-oxo-6,7,8,9, 10, 11, 12, 13, 14, 15-decahydro-3H-cyclopenta[a]phenanthren-17-yl)-2-oxoethyl acetate (INX-SM-28-1) (4.0 g, 9.523 mmol) and 1,4-dioxane (40 mL). To this reaction mixture, TFA (0.076 g, 0.666 mmol) and N-benzyl-1-methoxy-N-((trimethylsilyl)methyl) methanamine (22.5 g, 95.23 mmol) were added to the reaction mixture and stirred at 110° C. for 2 h. After completion of reaction as indicated by TLC, reaction mixture was poured into water and extracted with ethyl acetate. The combined organic layer was dried over Na₂SO₄ and concentrated under vacuum. The crude was purified by silica gel column chromatography (EtOAc/hexane, 32:68) to obtain title compound as white solid (3.5 g, 66.45%). LC/MS: 554.3 [M+H]⁺.

### Synthesis of 2-((2S,6aS,6bR,7S,8aS,8bS,11aR,12aS,12bS)-2,6b-difluoro-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-28-3)

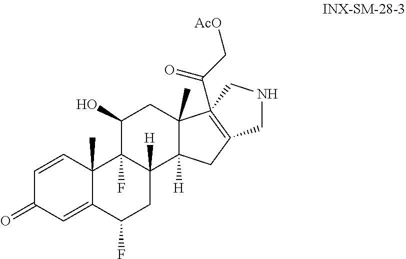

### Procedure

A 100 mL glass seal tube was charged with 2-((2S,6aS,6bR,7S,8aS,8bS,11aR,12aS,12bS)-10-benzyl-2,6b-difluoro-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-28-2) (3.5 g, 6.329 mmol) and acetonitrile (35 mL). To this solution, NaHCO₃ (1.06 g, 12.66 mmol) and 1-chloroethyl chloroformate (1.81 g, 12.658 mmol) were added at room temperature and the reaction mixture was stirred at 50° C. for 4 h. After completion of reaction as indicated by TLC, reaction mixture was diluted with EtOAc and washed with H₂O. The organic layer was dried by Na₂SO₄ and concentrated via under reduce pressure. The crude was purified by reverse phase column chromatography (acetonitrile:water, 25:75) to obtain title compound as white solid (1.6 g, 54.60%). LC/MS: 464.3 [M+H]⁺.

### Synthesis of (4-bromophenyl) (3-nitrophenyl) methanone (INX-SM-28-4)

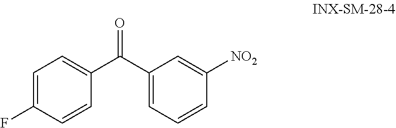 [SMILES: O=C(c1ccc(F)cc1)c1cccc([N+](=O)[O-])c1]

A 250 mL single-necked round bottom flask was charged with 3-nitrobenzoyl chloride (10.0 g, 0.054 mol) and bromobenzene (70 mL). To this solution, aluminium chloride (7.1 g, 0.054 mmol) was added at 0° C. and allowed to stir for 2 h at 80° C. After completion of reaction as indicated by TLC, reaction mixture was diluted with H₂O and extracted with ethyl acetate. The combined organic layer was dried over Na₂SO₄ and evaporated under vacuum. The crude was purified by silica gel column chromatography (ethyl acetate/hexane, 10:90) to give title compound as off white solid. (14.0 g, 84.64%). ¹H NMR (DMSO-d6) δ 8.51 (d, J=8.4 Hz, 1H), 8.43 (s, 1H), 8.16 (d, J=7.6 Hz, 1H), 7.87-7.71 (m, 5H).

### Synthesis of (4-bromophenyl) (3-nitrophenyl) methanol (INX-SM-28-5)

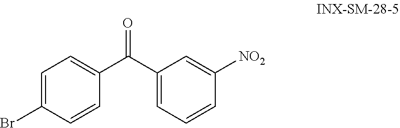 [SMILES: O=C(c1ccc(Br)cc1)c1cccc([N+](=O)[O-])c1]

### Procedure

A 100 mL single necked round bottom flask was charged with (4-bromophenyl) (3-nitrophenyl) methanone (INX-SM-28-4) (13.0 g, 0.042 mol) and THF (100 mL). To this solution, sodium borohydride (1.5 g, 0.042 mmol) was added at room temperature and stirred for another 2 h. After completion of reaction as indicated by TLC, reaction mixture was quenched with water and extracted with ethyl acetate. The combined organic layer was dried over Na₂SO₄ and evaporated under vacuum to give title compound as gummy solid (13.0 g, 99.35%). ¹H NMR (DMSO-d6) δ 8.24 (s, 1H), 8.10 (d, J=8.4 Hz, 1H), 7.81 (d, J=7.6 Hz, 1H), 7.60 (dd, J=8 Hz, 1H), 7.53 (d, J=8.4 Hz, 2H), 7.38 (d, J=8.4 Hz, 2H), 6.35 (brs, 1H), 5.88 (s, 1H).

### Synthesis of 1-(4-bromobenzyl)-3-nitrobenzene (INX-SM-28-6)

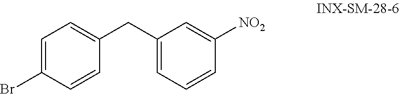 [SMILES: O=[N+]([O-])c1cccc(Cc2ccc(Br)cc2)c1]

### Procedure

A 250 mL single-necked round bottom flash was charged with (4-bromophenyl) (3-nitrophenyl) methanol (INX-SM-28-5) (14.0 g, 0.045 mol) and chloroform (50 mL). To this solution, triflic acid (27.0 g, 0.18 mol) and triethylsilane (5.27 g, 0.045 mol) were added at 0° C. and stirred for 1 h at 0° C. After completion of reaction as indicated by TLC, reaction mixture was quenched with NaHCO₃ solution and extracted with DCM. The combined organic layer was dried over Na₂SO₄ and evaporated under vacuum. The crude was purified by silica gel column chromatography (ethyl acetate/hexane: 10:90) to give title compound as off white solid (8.0 g, 60.8%). ¹H NMR (DMSO-d6) δ 8.11 (s, 1H), 8.08 (d, J=8.0 Hz, 1H), 7.72 (d, J=7.6 Hz, 1H), 7.59 (dd, J=8 Hz, 1H), 7.51 (d, J=8.4 Hz, 2H), 7.27 (d, J=8.0 Hz, 2H), 4.08 (s, 2H).

### Synthesis of 3-(4-bromobenzyl) aniline (INX-SM-28-7)

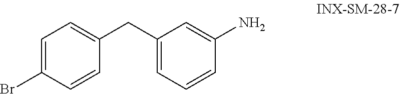 [SMILES: Nc1cccc(Cc2ccc(Br)cc2)c1]

### Procedure

A 250 mL single-necked round bottom flash was charged with 1-(4-bromobenzyl)-3-nitrobenzene (8.0 g, 0.027 mol) and ethanol: water (1:1, 100 mL) To this solution, Zn dust (14.0 g, 0.21 mmol) and (INX-SM-28-6) ammonium chloride (11.8 g, 0.21 mmol) were added at room temperature and stirred for 2 h. After completion of reaction as indicated by TLC, reaction mixture was filtered through celite and filtrate was evaporated under vacuum to give title compound as off white solid (7.0 g, 98.8%). LCMS: 262.1 [M+H]⁺.

### Synthesis of tert-butyl (3-(4-bromobenzyl)phenyl)carbamate (INX-SM-28-8)

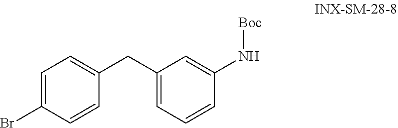 [SMILES: CC(C)(C)OC(=O)Nc1cccc(Cc2ccc(Br)cc2)c1]

### Procedure

A 10 mL single-necked round bottom flask was charged with 3-(4-bromobenzyl) aniline (INX-SM-28-7) (7.0 g, 0.026 mol) and THF (20 mL). To this solution, TEA (5.3 g, 0.053 mol) and boc anhydride (8.7 g, 0.040 mol) were added at 0° C. and allowed to stir at room temperature for 16 h. After completion of reaction as indicated by TLC, reaction mixture was quenched with water and extracted with ethyl acetate The combined organic layer was dried over Na₂SO₄ and evaporated under vacuum. The crude was purified by silica gel column chromatography (ethyl acetate/hexane: 10:90) to give title compound as off white solid (14 g, crude). It was taken to the next step without further purification. LCMS: 361.7 [M+H fragmentation].

### Synthesis of tert-butyl (3-(4-(4,4,5,5-tetramethyl-1,3,2-dioxaborolan-2-yl)benzyl)phenyl)carbamate (INX-SM-28-9)

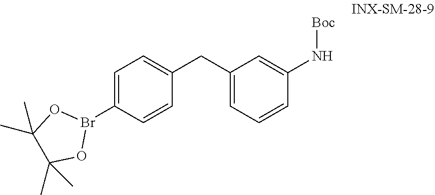

### Procedure

A 25 mL single necked round bottom flask was charged with tert-butyl (3-(4-bromobenzyl)phenyl)carbamate (INX-SM-28-8) (14.0 g, 0.038 mol) and 1,4-dioxane (30 mL) under nitrogen. To this solution, bispinacolonediborane (14.4 g, 0.057 mol) and potassium acetate (7.4 g, 0.076 mol) were added and reaction mixture was urged with N₂ for 15 min. PdCl₂ (dppf)·DCM (3.1 g, 0.0038 mol) was added and stirred at 110° C. for 2 h. After completion of reaction as indicated by TLC, reaction mixture was cooled at room temperature, diluted with ethyl acetate and filtered through celite bed. The combined organic layer was dried over Na₂SO₄ and evaporated under vacuum. The crude was purified by silica gel column chromatography (ethyl acetate/hexane: 20:80) to give title compound as off white semisolid (20.0 g, crude). LCMS: 410.4 [M+H]⁺.

### Synthesis of (4-(3-((tert-butoxycarbonyl)amino)benzyl)phenyl)boronic acid (INX-SM-28-10)

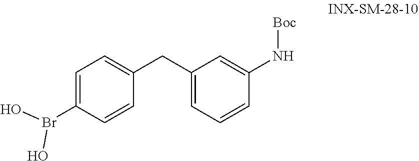

### Procedure

A 1000 mL single-necked round bottom flask was charged with tert-butyl (3-(4-(4,4,5,5-tetramethyl-1,3,2-dioxaborolan-2-yl)benzyl)phenyl)carbamate (INX-SM-28-9) (20.0 g, 0.048 mol) and acetone: water (9:1, 200 mL). To this solution, NaIO₄ (81.7 g, 0.38 mol) and ammonium acetate (29.26 g, 0.38 mol) were added heated at reflux for 2 h. After completion of reaction as indicated by TLC, reaction mixture was cooled to room temperature, diluted with ethyl acetate and filtered through celite. The combined organic layer was dried over Na₂SO₄ and evaporated under vacuum. The crude was triturated with n-pentene and diethyl ether to give title compound as off white solid (15.0 g, 95.5%). LCMS: 328.2 [M+H]⁺

### Synthesis of 2-((2S,6aS,6bR,7S,8aS,8bS,11aR,12aS,12bS)-10-(4-(3-((tert-butoxycarbonyl)amino)benzyl)phenyl)-2,6b-difluoro-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-28-11)

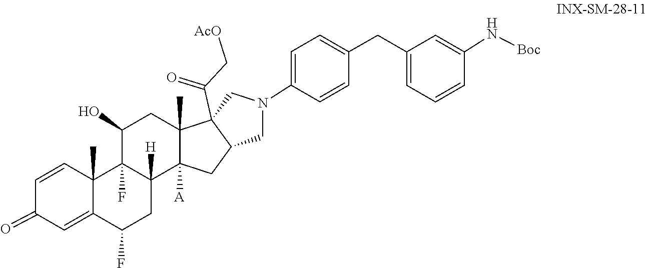

### Procedure

A 35 mL glass vial was charged with 2-((2S,6aS,6bR,7S,8aS,8bS,11aR,12aS,12bS)-2,6b-difluoro-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-28-3) (0.320 g, 0.691 mmol) and acetonitrile (35 mL). To this solution, (4-(3-((tert-butoxycarbonyl)amino)benzyl)phenyl)boronic acid (INX-SM-28-10) (1.01 g, 3.109 mmol), KOH (0.387 g, 6.910 mmol) and Cu (OAC) 2 (0.375 g, 2.073 mmol) were added stirred at 60° C. for 1 h. After completion of reaction as indicated by TLC, reaction mixture was diluted with EtOAc and filtered through celite and concentrated under vacuum. The crude was purified by silica column chromatography (ethyl acetate/hexane, 30:70) to give title compound as yellow solid (0.095 g, 18.59%).

LC/MS: 745.4 [M+H]⁺.

### Synthesis of tert-butyl (3-(4-((2S,6aS,6bR,7S,8aS,8bS,11aR,12aS,12bS)-2,6b-difluoro-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)carbamate (INX-SM-28-12)

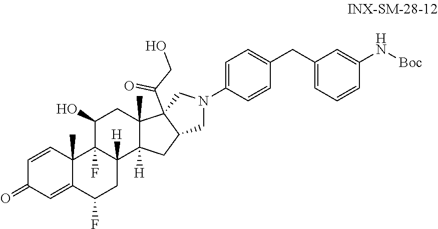

### Procedure

A 10 mL single necked round bottom flask was charged with 2-((2S,6aS,6bR,7S,8aS,8bS,11aR,12aS,12bS)-10-(4-(3-((tert-butoxycarbonyl)amino)benzyl)phenyl)-2,6b-difluoro-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-28-11) (0.095 g, 0.127 mmol) and methanol (2 mL). To this solution, potassium carbonate (0.027 g, 0.191 mmol) was added and the reaction mixture was stirred at room temperature for 30 min. After completion of reaction as indicated by TLC, reaction mixture was diluted with EtOAc and washed with H₂O. The combined organic layer was dried over Na₂SO₄ and concentrated under vacuum to get crude title compound product as yellow solid (0.085 g, 95.2%). LCMS: 703.4 [M+H]⁺

### Synthesis of (2S,6aS,6bR,7S,8aS,8bS,11aR,12aS,12bS)-10-(4-(3-aminobenzyl)phenyl)-2,6b-difluoro-7-hydroxy-8b-(2-hydroxyacetyl)-6a,8a-dimethyl-1,6a,6b,7,8,8a,8b,9,10,11,11a,12,12a,12b-tetradecahydronaphtho[2′,1′:4,5]indeno[1,2-c]pyrrol-4 (2H)-one (INX-SM-28)

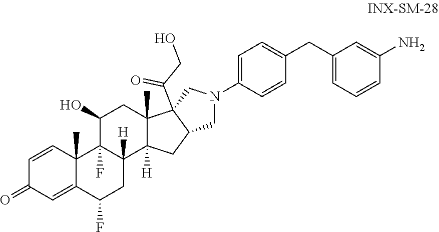

### Procedure

A 10 mL single necked round bottom flask was charged with tert-butyl (3-(4-((2S,6aS,6bR,7S,8aS,8bS,11aR,12aS,12bS)-2,6b-difluoro-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)carbamate (INX-SM-28-12) (0.085 g, 0.121 mmol) and DCM (2 mL). To this solution, TFA (0.1 mL) was added and stirred at room temperature for 1 h. After completion of reaction as indicated by TLC, reaction mixture was concentrated under vacuum. The crude was purified by prep HPLC (Column: C18, 250*21.2 mm, 5 μm, Mobile phase A=0.05% trifluoroacitic acid in water, B=acetonitrile:methanole:IPA (65:25:10), A:B=65:35; retention time 15.32 min) to obtain title compound as yellow solid (0.005 g, 6.86%). LC/MS: 603.3 [M+H]⁺

### Synthesis of (6aR,6bS,7S,8aS,8bS,11aR,12aS,12bS)-10-(4-(3-aminobenzyl)phenyl)-7-hydroxy-8b-(2-hydroxyacetyl)-6a,8a-dimethyl-1,6a,6b,7,8,8a,8b,9,10,11,11a,12,12a,12b-tetradecahydronaphtho[2′,1′:4,5]indeno[1,2-c]pyrrol-4 (2H)-one (INX-SM-40) 1>

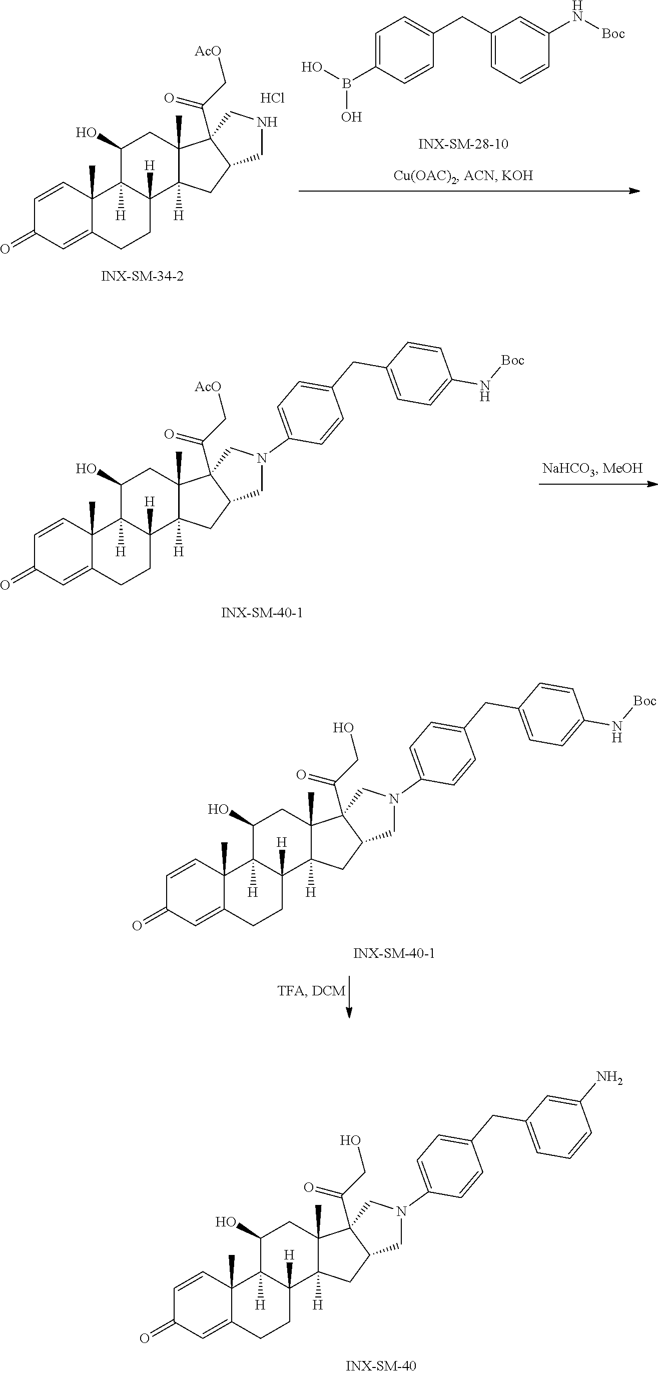

### Synthesis of 2-((6aR,6bS,7S,8aS,8bS,11aR,12aS,12bS)-10-(4-(4-((tert-butoxycarbonyl)amino)benzyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-40-1)

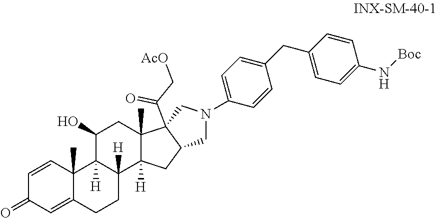

### Procedure

A 35 mL glass vial was charged with 2-((6aR,6bS,7S,8aS,8bS,11aR,12aS,12bS)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate hydrochloride (INX-SM-34-2) (0.4 g, 0.93 mmol) and (4-(3-((tert-butoxycarbonyl)amino)benzyl)phenyl)boronic acid (INX-SM-28-10) (1.07 g, 3.27 mmol) in acetonitrile (40 mL). To this solution, KOH (0.52 g, 9.36 mmol) and Cu (OAc) 2 (0.50 g, 2.81 mmol) were added and the reaction mixture was stirred at room temperature for 3 h. After completion of reaction as indicated by TLC, reaction mixture was diluted with ethyl acetate and filtered through celite. The combined organic layer was dried over Na₂SO₄ and evaporated under vacuum. The crude was purified by silica gel column chromatography (ethyl acetate: hexane, 30:70) to give title compound as off white solid (0.1 g, 15.8%). LCMS: 709.5 [M+H]⁺.

### Synthesis of tert-butyl (4-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)carbamate (INX-SM-40-2)

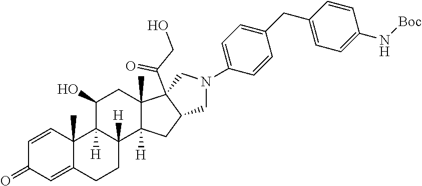

### Procedure

A 25 mL single-necked round bottom flask was charged with 2-((6aR,6bS,7S,8aS,8bS,11aR,12aS,12bS)-10-(4-(4-((tert-butoxycarbonyl)amino)benzyl)phenyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate (INX-SM-40-1) (0.13 g, 0.18 mmol) and methanol: water (2 mL). To this solution, NaHCO₃ (0.030 g, 0.36 mmol) was added stirred the reaction mixture at room temperature for 12 h. After completion of reaction as indicated by TLC, reaction mixture was diluted with ethyl acetate and filtered through celite. The combined organic layer was evaporated to give crude INX-SM-40-2 product as off white solid (0.06 g 49.98%). MS: 667.4 [M+H]⁺.

### Synthesis of (6aR,6bS,7S,8aS,8bS,11aR,12aS,12bS)-10-(4-(3-aminobenzyl)phenyl)-7-hydroxy-8b-(2-hydroxyacetyl)-6a,8a-dimethyl-1,6a,6b,7,8,8a,8b,9,10,11,11a,12,12a,12b-tetradecahydronaphtho[2′,1′:4,5]indeno[1,2-c]pyrrol-4 (2H)-one (INX-SM-40)

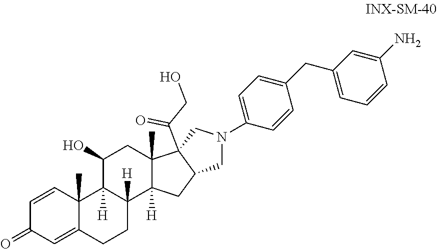

### Procedure

A 10 mL single-necked round bottom flask was charged with tert-butyl (4-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)carbamate INX-SM-40-2) (0.06 g, 0.08 mmol) and DCM (2 mL). To this solution, TFA (0.12 mL) was added and stirred the reaction mixture for 2 h at room temperature. After completion of reaction as indicated by TLC, reaction mixture was evaporated under vacuum. The crude was purified by prep-HPLC (Column: YMC-Actus Triart Prep C18-S, 250×20 mm S-5 μm, 12 nm, Mobile phase: A=0.05% TFA in water, B=Acetonitrile, A:B=66:34, retention time 14.5 min) to give title compound as off white solid (0.004 g, 8.82%) LCMS: 567 [M+H]⁺; ¹H NMR (400 MHZ, MeOD: δ: 7.48 (d, J=10 Hz, 1H), 7.39 (t, J=8.0 Hz, 1H), 7.25 (d, J=7.6 Hz, 1H), 7.11 (d, J=7.6 Hz 1H), 7.06 (s, 1H), 7.03 (d, J=8.4 Hz, 2H), 6.62 (d, J=8.4 Hz, 2H), 6.26 (dd, J=10.0 & 2.0 Hz, 1H), 5.99 (s, 1H), 4.60-4.30 (m, 3H), 3.91 (s, 2H), 3.60-3.00 (m, 4H), 2.70-1.55 (m, 10H), 1.50 (s, 3H), 1.10 (s, 3H), 1.10-0.95 (m, 2H).

### Synthesis of (6aR,6bS,7S,8aS,8bS,10R,11aR,12aS,12bS)-10-(4-((2-azaspiro[3.3]heptan-6-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-47)

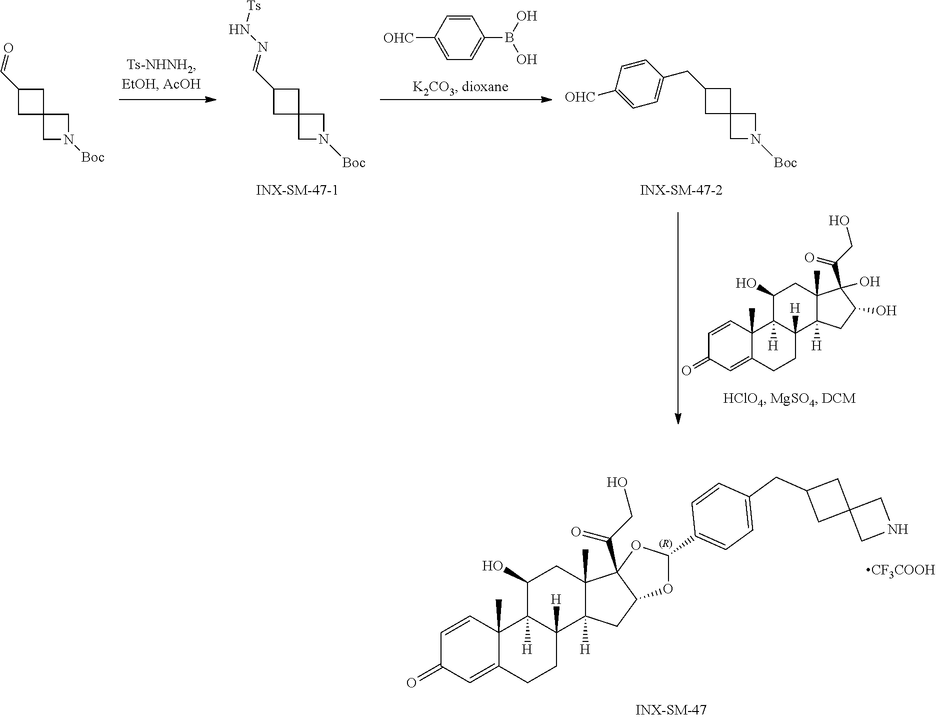

### Synthesis of tert-butyl (E)-6-((2-tosylhydrazono)methyl)-2-azaspiro[3.3]heptane-2-carboxylate (INX-SM-47-1)

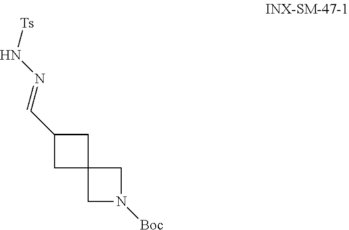 [SMILES: Cc1ccc(S(=O)(=O)N/N=C/C2CC3(C2)CN(C(=O)OC(C)(C)C)C3)cc1]

### Procedure

A 35 mL glass vial was charged with tert-butyl 6-formyl-2-azaspiro[3.3]heptane-2-carboxylate (0.2 g, 0.880 mmol) and EtOH (10 mL) under nitrogen atmosphere. To this solution, p-toluenesulfonhydrazide (0.247 g, 1.33 mmol) and catalytic AcOH (0.1 mL) were added and stirred for 0.5h at room temperature. After completion of reaction as indicated by TLC, reaction mixture was poured into water and solid precipitates was filtered and dried under vacuum to give title compound as white solid (0.3 g, 85.88%). LCMS: 394.2 [M+H]+.

### Synthesis of tert-butyl 6-(4-formylbenzyl)-2-azaspiro[3.3]heptane-2-carboxylate (INX-SM-47-2)

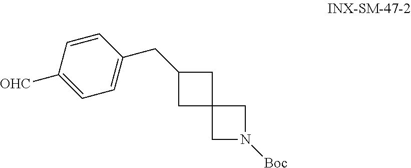 [SMILES: CC(C)(C)OC(=O)N1CC2(CC(Cc3ccc(C=O)cc3)C2)C1]

A 35 mL glass vial was charged with tert-butyl (E)-6-((2-tosylhydrazono)methyl)-2-azaspiro[3.3]heptane-2-carboxylate (INX-SM-47-1) (0.3 g, 0.762 mmol) and dioxane (5 mL) under nitrogen atmosphere. To this solution, (4-formylphenyl)boronic acid (0.171 g, 1.14 mmol) and K2CO3 (0.158 g, 1.14 mmol) were added at room temperature and stirred for another 3 h at 100° C. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane: 50:50) to give title compound as yellow gummy solid (0.065 g, 27.86%). LCMS: 316.2 [M+H]+.

### Synthesis of (6aR,6bS,7S,8aS,8bS,10R,11aR,12aS,12bS)-10-(4 ((2-azaspiro[3.3]heptan-6-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-47)

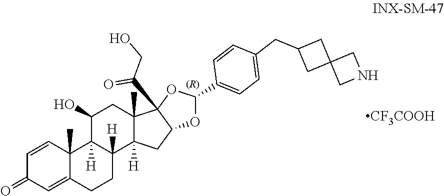

### Procedure

A 25 mL single-necked round bottom flash was charged with tert-butyl 6-(4-formylbenzyl)-2-azaspiro[3.3]heptane-2-carboxylate (INX-SM-47-2) (0.065 g, 0.206 mmol), (8S,9S,10R,11S,13S,14S,16R,17S)-11,16,17-trihydroxy-17-(2-hydroxyacetyl)-10,13-dimethyl-6,7,8,9,10,11,12,13,14,15, 16, 17-dodecahydro-3H-cyclopenta[a]phenanthren-3-one (16-alpha-hydroxyprednisolone) (0.077 g, 0.206 mmol) and DCM (2 mL). To this solution, MgSO₄ (0.103 g, 1.03 mmol) and HClO₄ (0.123 g, 1.03 mmol) were added and stirred for another 1.5h at room temperature. After completion of reaction as indicated by TLC, reaction mixture was quenched with sat. NaHCO₃ solution and concentrated under vacuum. The crude was triturated with cold water and participated was filtered and dried under vacuum. The crude was purified by prep-HPLC (Column: YMC-PACK ODS-AQ Prep C18-S, 250×20 mm S-5 μm, 12 nm, Mobile phase: A=0.05% TFA in water, B=Acetonitrile:Methanol:2-Propanol (65:25:10), A:B=58:42, retention time 12.23 min) to give title compound as white solid (0.025 g, 17.77%). LCMS: 574.5 [M+H]+; ¹H NMR (400 MHZ, MeOD): □: 7.47 (d, J=10.0 Hz, 1H), 7.37 (d, J=8.0 Hz, 2H), 7.18 (d, J=8.4 Hz, 2H), 6.27 (dd, J=10.0& 2.0 Hz, 1H), 6.04 (s, 1H), 5.46 (s, Acetal H, 1H), 5.07 (d, J=5.2 Hz, C16-H 1H), 4.63 (d, J=19.6 Hz, 1H), 4.45-4.44 (s, 1H), 4.33 (d, J=19.6 Hz 1H), 4.07 (s, 2H), 3.95 (s, 2H), 2.75-1.73 (m, 16H), 1.50 (s, 3H), 1.05-1.05 (m, 2H), 1.01 (s, 3H).

### Synthesis of (S)-4-(2-(2-bromoacetamido)acetamido)-5-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-azaspiro[3.3]heptan-2-yl)-5-oxopentanoic acid (INX-A19)

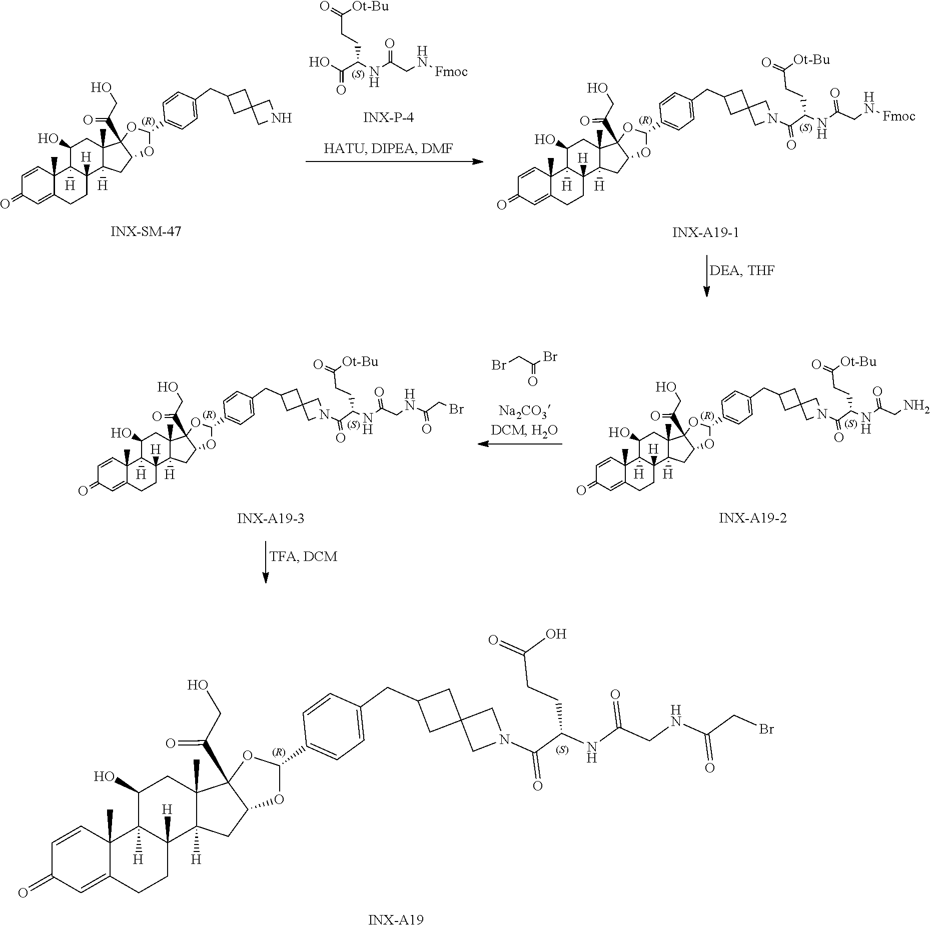

### Synthesis of tert-butyl(S)-4-(2-((((9H-fluoren-9-yl)methoxy)carbonyl)amino)acetamido)-5-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-azaspiro[3.3]heptan-2-yl)-5-oxopentanoate (INX-A19-1)

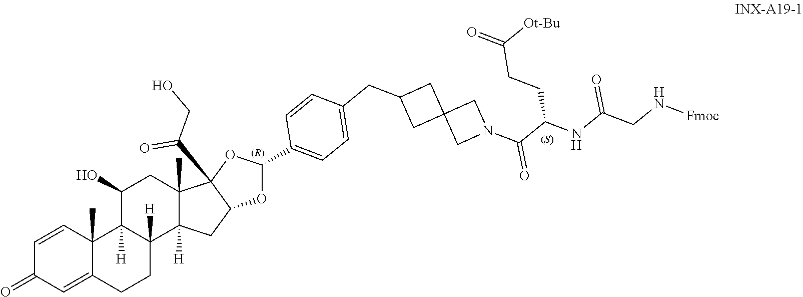

### Procedure

A 50 mL single-necked round bottom flash was charged(S)-2-(2-((((9H-fluoren-9-yl)methoxy)carbonyl)amino)acetamido)-5-(tert-butoxy)-5-oxopentanoic acid (INX-P-4) (0.605 g, 1.25 mmol) and HATU (0.596 g, 1.56 mmol) in DMF (6 mL). To this solution, DIPEA (0.405 g, 3.13 mmol) followed by 6aR,6bS,7S,8aS,8bS,10R,11aR,12aS,12bS)-10-(4-((2-azaspiro[3.3]heptan-6-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-47) (0.6 g, 1.04 mmol) were added at room temperature and stirred for 2 h at room temperature. After completion of reaction as indicated by TLC, reaction mixture was poured into water and extracted with ethyl acetate. The combined organic layer was dried over Na₂SO₄ and evaporated under vacuum to give crude product. The crude was purified by reverse phase column chromatography (ACN: Water, 70:30) to give title compound as white solid (0.20 g, 18.42%). LCMS: 1038.8 [M+H]+.

### Synthesis of tert-butyl(S)-4-(2-aminoacetamido)-5-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-azaspiro[3.3]heptan-2-yl)-5-oxopentanoate (INX-A19-2)

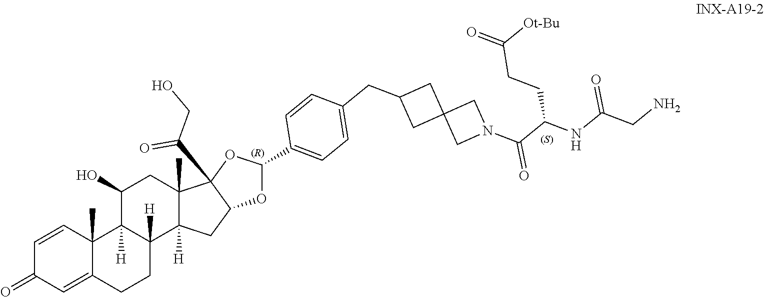

### Procedure

A 25 mL single-necked round bottom flash was charged with tert-butyl(S)-4-(2-((((9H-fluoren-9-yl)methoxy)carbonyl)amino)acetamido)-5-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a, 8b,11a,12,12a,12b-dodecahydro-1H-naphtho[2′,1′:4,5]indeno[1,2-d][1,3]dioxol-10-yl)benzyl)-2-azaspiro[3.3]heptan-2-yl)-5-oxopentanoate (INX-A19-1) (0.2 g, 0.192 mmol) and THF (2 mL). To this solution, diethylamine (0.140 g, 1.92 mmol) was added and stirred for 2.5h at room temperature. After completion of reaction as indicated by TLC, reaction mixture was concentrated under vacuum. The crude was triturated with DCM and hexane to give title compound as white solid (0.12 g, 76.34%). LCMS: 816.6 [M+H]+.

### Synthesis of tert-butyl(S)-4-(2-(2-bromoacetamido)acetamido)-5-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-azaspiro[3.3]heptan-2-yl)-5-oxopentanoate (INX-A19-3)

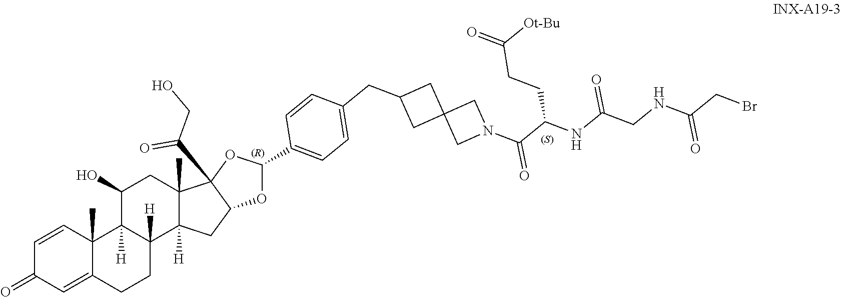

### Procedure

A 25 mL single-necked round bottom flash was charged with tert-butyl(S)-4-(2-aminoacetamido)-5-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-azaspiro[3.3]heptan-2-yl)-5-oxopentanoate (INX-A19-2) (0.120 g, 0.147 mmol) and DCM (1 mL). To this solution, Na₂CO₃ (0.031 g, 0.294 mmol) dissolved in water (0.1 mL) followed by bromoacetyl bromide (0.029 g, 0.147 mmol) were added at room temperature and stirred for 0.5h. After completion of reaction as indicated by TLC, reaction mixture was quenched with water and extracted with DCM. The combined organic layer was dried over Na₂SO₄ and evaporated under vacuum to give title compound as off white solid (0.095 g, 68.95%). LCMS: calculated for C48H6379BrFN3O11 (936.36), found 936.4 [M+H]+.

### Synthesis of (S)-4-(2-(2-bromoacetamido)acetamido)-5-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-azaspiro[3.3]heptan-2-yl)-5-oxopentanoic acid (INX-A19)

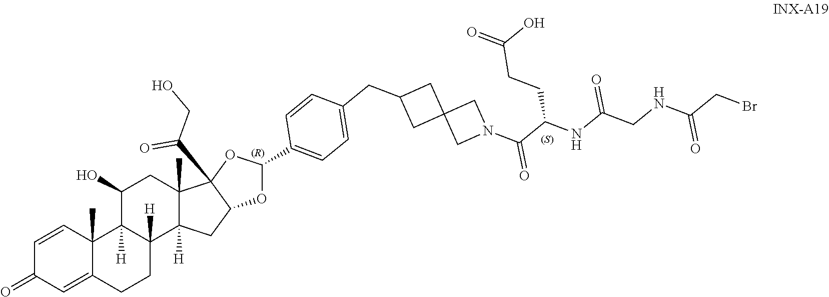

### Procedure

A 25 mL single-necked round bottom flash was charged with tert-butyl(S)-4-(2-(2-bromoacetamido)acetamido)-5-(6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)-2-azaspiro[3.3]heptan-2-yl)-5-oxopentanoate (INX-A19-3) (0.095 g, 0.101 mmol) in DCM (3 mL). To this solution, TFA (0.2 ml) was added at room temperature and stirred for 2 h. After completion of reaction as indicated by TLC, reaction mixture was evaporated under vacuum. The crude was purified by prep-HPLC (Column: NEW X-bridge Prep, C18, OBD (250×19) mm, 5 μm, Mobile phase: A=0.05% TFA in water, B=ACN, A:B=68:32, retention time 13.59 min) to give title compound as white solid (0.006 g, 6.72%). LCMS: calculated for C44H5579BrFN3O11 (880.3), found 880.2 [M+H]+; ¹H NMR (400 MHZ, MeOD): 0 7.47 (d, J=10.0 Hz, 1H), 7.36 (d, J=7.6 Hz, 2H), 7.20-7.17 (m, 2H), 6.27 (dd, J=10.0 & 2.0 Hz, 1H), 6.04 (s, 1H), 5.45 (s, 1H), 5.06 (d, J=5.2 Hz, 1H), 5.00-3.80 (m, 12H), 2.75-1.65 (m, 20H), 1.51 (s, 3H), 1.18-1.05 (m, 2H), 1.03 (s, 3H).

### Synthesis of (2S,6aS,6bR,7S,8aS,8bS,10R,11aR,12aS,12bS)-10-(4-(((1r,4R)-4-aminocyclohexyl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-14)

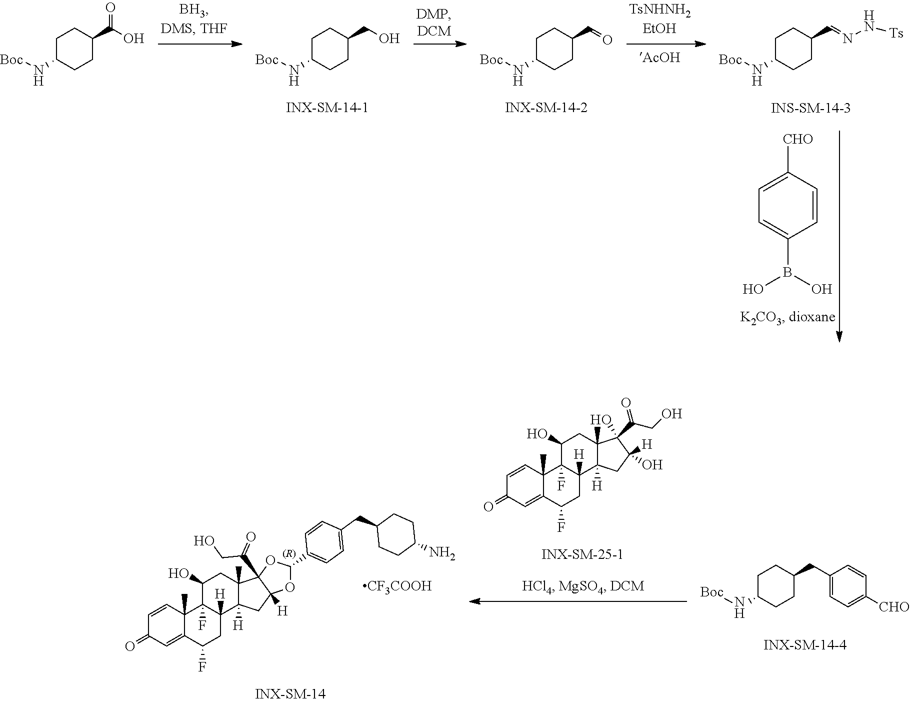

### Synthesis of tert-butyl ((1r,4r)-4-(hydroxymethyl)cyclohexyl)carbamate (INX-SM-14-1)

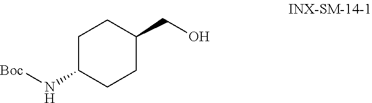 [SMILES: CC(C)(C)OC(=O)N[C@H]1CC[C@H](CO)CC1]

### Procedure

In a 100 mL single-necked round bottom flask, (1r,4r)-4-((tert-butoxycarbonyl)amino)cyclohexane-1-carboxylic acid (3.0 g, 12.34 mmol) was dissolved in THF (30 mL) and borane dimethyl sulfide (BH3·DMS) (3.0 mL, 6.17 mmol) was added at 0° C. The reaction mixture was allowed to stir at 0° C. for 30 min. After completion of reaction as indicated by TLC, reaction mixture was quenched with dil. and extracted with ethyl acetate. The combined organic layer was washed with brine, dried over Na₂SO₄ and evaporated under vacuum to give title compound as white solid (3.0 g, crude). It was used for next step without purification. LCMS: 230.2 [M+H]+.

### Synthesis of tert-butyl ((1r,4r)-4-formylcyclohexyl)carbamate (INX-SM-14-2)

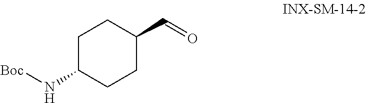 [SMILES: CC(C)(C)OC(=O)N[C@H]1CC[C@H](C=O)CC1]

### Procedure

In a 100 mL single-necked round bottom flask, a solution of tert-butyl ((1r,4r)-4-(hydroxymethyl)cyclohexyl)carbamate (INX-SM-14-1) (3.0 g, 13.08 mmol) in DCM (30 mL) was added. To this solution, DMP (6.6 g, 15.72 mmol) was added at 0° C. and stirred for another 1 h at room temperature. After completion of reaction as indicated by TLC, reaction mixture was quenched with NaHCO₃ solution and extracted with DCM. The combined organic layer was washed with brine, dried over Na₂SO₄ and evaporated under vacuum to give crude title compound as white solid (3.0 g, Crude). It was directly used in next step without analysis.

### Synthesis of tert-butyl ((1r,4r)-4-((E)-(2-tosylhydrazono)methyl)cyclohexyl)carbamate (INX-SM-14-3)

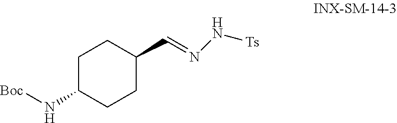 [SMILES: Cc1ccc(S(=O)(=O)N/N=C/[C@H]2CC[C@H](NC(=O)OC(C)(C)C)CC2)cc1]

### Procedure

A 100 mL single-necked round bottom flask was charged with tert-butyl ((1r,4r)-4-formylcyclohexyl)carbamate (INX-SM-14-2) (3.0 g, 13.21 mmol) and ethanol (30 mL). To this solution, p-Toluenesulfonhydrazide (3.6 g, 19.82 mmol) and catalytic amount of AcOH (0.1 mL) were added and stirred for 1 h at room temperature. After completion of reaction as indicated by TLC, reaction mixture was poured into water and the formed solid was filtered and dried under vacuum to give title compound as white solid (3.5 g, 67.05%). LCMS: 396.2 [M+H]+.

### Synthesis of tert-butyl ((1r,4r)-4-(4-formylbenzyl)cyclohexyl)carbamate (INX-SM-14-4)

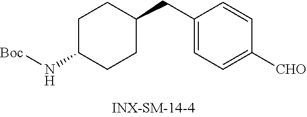 [SMILES: CC(C)(C)OC(=O)N[C@H]1CC[C@H](Cc2ccc(C=O)cc2)CC1]

### Procedure

A 100 mL single-necked round bottom flask was charged with tert-butyl ((1r,4r)-4-((E)-(2-tosylhydrazono)methyl)cyclohexyl)carbamate (INX-SM-14-3) (2.0 g, 5.06 mmol) and dioxane (20 mL). To this solution, K2CO3 (1.0 g, 7.50 mmol) was added and the reaction mixture was purged with N₂ for 20 min. To this solution, (4-formylphenyl)boronic acid (1.13 g, 7.58 mmol) was added and refluxed for another 2 h. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane: 50:50) to give title compound as yellow sticky solid (0.6 g, 37.38%). LCMS: 318.3 [M+H]+.

### Synthesis of (2S,6aS,6bR,7S,8aS,8bS,10R,11aR,12aS,12bS)-10-(4-(((1r,4R)-4-aminocyclohexyl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-14)

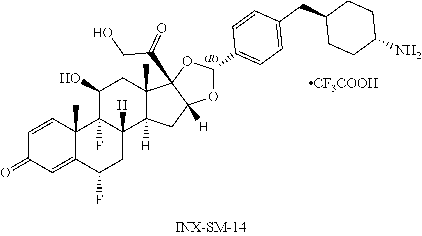

### Procedure

A 10 ml glass vial was charged with tert-butyl ((1r,4r)-4-(4-formylbenzyl)cyclohexyl)carbamate (INX-SM-14-4) (0.10 g, 0.31 mmol) and (6S,8S,9R,10S,11S,13S,14S,16R,17S)-6,9-difluoro-11,16,17-trihydroxy-17-(2-hydroxyacetyl)-10, 13-dimethyl-6,7,8,9, 10, 11, 12, 13, 14, 15, 16, 17-dodecahydro-3H-cyclopenta[a]phenanthren-3-one (INX-SM-25-1) (0.13 g, 0.31 mmol) in DCM (2 mL). To this solution, MgSO4 (0.18 g, 1.50 mmol) and HClO4 (0.26 g, 2.6 mmol) were added at 0° C. and allowed to stir at room temperature for 2 h. After completion of reaction as indicated by TLC, reaction mixture was quenched with sat. NaHCO3 solution and extracted with DCM. The combined organic layer was dried over Na2SO4 and evaporated under vacuum. The crude was purified by prep-HPLC (Column: YMC-Actus Triart Prep C18-S, 250×20 mm S-5 μm, 12 nm, Mobile phase: A=0.05% TFA in water, B=Acetonitrile; A:B=70:30, retention time 15.92 min) to give title compound as white solid (0.015 g, 7.78%). LCMS: 612.3 [M+H]+; ¹H NMR (400 MHZ, DMSO-d6): □ 7.62 (brs, 3H), 7.35 (d, J=8 Hz, 2H), 7.27 (d, J=9.6 Hz, 1H), 7.18 (d, J=8 Hz, 2H), 6.30 (dd, J=10 Hz, 1H), 6.13 (s, 1H), 5.70-5.60 (m, 1H), 5.56 (br s, 1H), 5. 5.46 (s, Acetal-H, 1H), 5.14 (t, 1H), 5.09 (d, J=4.4 Hz, C16-H, 1H), 4.53-4.48 (m, 1H), 4.24-4.18 (m, 2H), 2.94-2.88 (m, 1H), 2.60-1.40 (m, 15H), 1.45 (s, 3H), 1.23-0.98 (m, 4H), 0.87 (s, 3H).

### Synthesis Scheme of (2S,6aS,6bR,7S,8aS,8bS,10R,11aR,12aS,12bS)-10-(4-(((1s,4S)-4-aminocyclohexyl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-15)

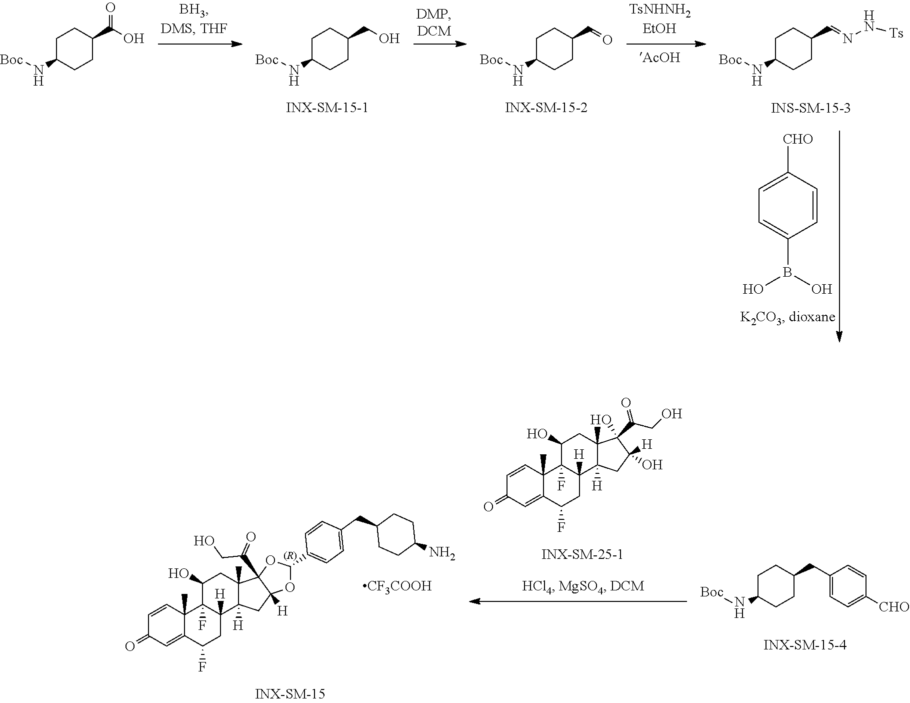

### Synthesis of tert-butyl ((1s, 4s)-4-hydroxymethyl)cyclohexyl)carbamate (INX-SM-15-1)

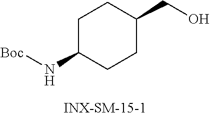 [SMILES: CC(C)(C)OC(=O)N[C@H]1CC[C@@H](CO)CC1]

### Procedure

A 50 ml round bottom flask was charged with (1s,4s)-4-((tert-butoxycarbonyl)amino)cyclohexane-1-carboxylic acid (2.5 g, 10.28 mmol) and THF (25 mL). To this solution, borane·DMS (2M in THF) (12.34 mL, 30.60 mmol) was added at 0° C. and stirred for another 1 h at room temperature. After completion of reaction as indicated by TLC, reaction mixture was quenched with MeOH and dil. HCl. The reaction mixture was extracted with ethyl acetate. The combined organic layer was dried over Na₂SO₄ and evaporated under vacuum to give title compound as white solid (2.3 g, 97.61%). ¹H NMR (400 MHZ, DMSO-d6): δ 6.67 (d, J=7.2 Hz, 1H), 4.35 (t, J=5.6 Hz, 1H), 3.44 (br s, 1H), 3.25 (t, J=6.0 Hz, 1H), 1.50-1.43 (m, 19H).

### Synthesis of tert-butyl ((1s, 4s)-4-formylcyclohexyl)carbamate (INX-SM-15-2)

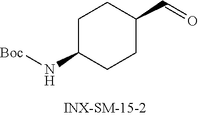 [SMILES: CC(C)(C)OC(=O)N[C@H]1CC[C@@H](C=O)CC1]

### Procedure

A 50 ml single necked round bottom flask was charged with tert-butyl ((1s, 4s)-4-hydroxymethyl)cyclohexyl)carbamate (INX-SM-15-1) (2.3 g, 10.03 mmol) and DCM (25 mL). TO this solution, DMP (6.4 g, 15.09 mmol) was added at room temperature and stirred for another 2 h. After completion of reaction as indicated by TLC, reaction mixture was quenched with NaHCO₃ solution and extracted with DCM. The combined organic layer was washed with brine, dried over Na₂SO₄ and evaporated under vacuum to give title compound as pale yellow gummy solid (2.3 g crude). It was used in next step without analysis.

### Synthesis of tert-butyl ((1s,4s)-4-((E)-(2-tosylhydrazono)methyl)cyclohexyl)carbamate (INX-SM-15-3)

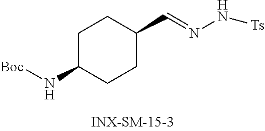 [SMILES: Cc1ccc(S(=O)(=O)N/N=C/[C@H]2CC[C@@H](NC(=O)OC(C)(C)C)CC2)cc1]

A 25 mL single necked round bottom flask was charge with tert-butyl ((1s, 4s)-4-formylcyclohexyl)carbamate (INX-SM-15-2) (2.3 g, 10.12 mmol) and EtOH (25 mL). To this solution, p-Toluenesulfonhydrazide (1.88 g, 10.12 mmol) and acetic acid (cat.) were added and stirred for another 2 h at room temperature. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane, 1:1) to give title compound as white solid (2.5 g, 62.47%). LCMS: 394.4 [M−H]+.

### Synthesis tert-butyl ((1s, 4s)-4-(4-formylbenzyl)cyclohexyl)carbamate (INX-SM-15-4)

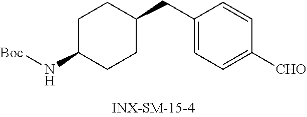 [SMILES: CC(C)(C)OC(=O)N[C@H]1CC[C@@H](Cc2ccc(C=O)cc2)CC1]

### Procedure

A 25 mL single necked round bottom flask was charged with tert-butyl ((1s,4s)-4-((E)-(2-tosylhydrazono)methyl)cyclohexyl)carbamate (INX-SM-15-3) (0.85 g, 2.15 mmol) and dioxane (10 mL). To this solution, (4-formylphenyl)boronic acid (0.30 g, 2.02 mmol) and K₂CO3 (0.45 g, 3.23 mmol) were added and refluxed for another 2 h at 100° C. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane, 1:1) to give title compound as pale yellow gummy solid (0.15 g, 21.99%). LCMS: 318.3 [M+H]+.

### Synthesis of (2S,6aS,6bR,7S,8aS,8bS,10R,11aR,12aS,12bS)-10-(4-(((1s,4S)-4-aminocyclohexyl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-15)

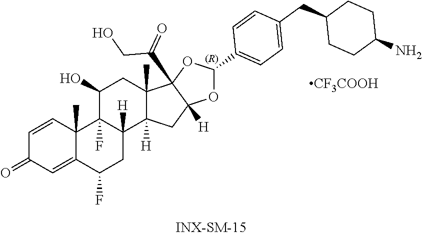

### Procedure

A 10 mL single necked round bottom flask was charge with tert-butyl ((1s,4s)-4-(4-formylbenzyl)cyclohexyl)carbamate (INX-SM-15-4) (0.15 g, 0.47 mmol) and DCM (3 mL). To this solution, (6S,8S,9R,10S,11S,13S,14S,16R,17S)-6,9-difluoro-11,16,17-trihydroxy-17-(2-hydroxyacetyl)-10, 13-dimethyl-6,7,8,9, 10, 11, 12, 13, 14, 15, 16, 17-dodecahydro-3H-cyclopenta[a]phenanthren-3-one (INX-SM-25-1) (0.15 g, 0.37 mmol), MgSO4 (0.28g 2.36 mmol) and HClO4 (0.23 g, 2.36 mmol) were added and stirred for 3 h at room temperature. After completion of reaction as indicated by TLC, reaction mixture was quenched with NaHCO₃ and extracted with ethyl acetate. The combined organic layer was dried over Na2SO4 and evaporated under vacuum. The crude was purified by prep-HPLC (Column: NEW X-bridge Prep, C18, OBD (250×19) mm, 5 μm, Mobile phase: A=0.05% TFA in water, B=acetonitrile; A:B=80:20, retention time 10.00 min) to give title compound as white solid (0.030 g, 10.38%). LCMS: 612.2 [M+H]+; ¹H NMR (400 MHZ, DMSO-d6): □: 7.69 (br s, 3H), 7.36 (d, J=10.0, 2H), 7.28 (d, J=10.2, 1H), 7.19 (d, J=10.0, 2H), 6.30 (dd, J=10.0 & 2.0 Hz, 2H), 5.74-5.60 (m, 2H), 5.56 (br s, 1H), 5.46 (s, Acetal-H, 1H), 5.14 (t, J=6.0, 1H), 4.96 (d, J=4.4 Hz, C16-H, 1H), 4.65-4.20 (m, 3H), 3.25-3.10 (m, 1H), 2.62-2.1.60 (m, 12H), 1.50 (s, 3H), 1.46-1.30 (m, 4H), 1.20 (s, 3H).

### Synthesis Scheme of (6aR,6bS,7S,8aS,8bS,11aR,12aS,12bS)-10-(4-((3-aminobicyclo[1.1.1]pentan-1-yl)methyl)benzyl)-7-hydroxy-8b-(2-hydroxyacetyl)-6a,8a-dimethyl-1,6a,6b,7,8,8a,8b,9,10,11,11a,12,12a,12b-tetradecahydronaphtho[2′,1′:4,5]indeno[1,2-c]pyrrol-4 (2H)-one (INX-SM-17)

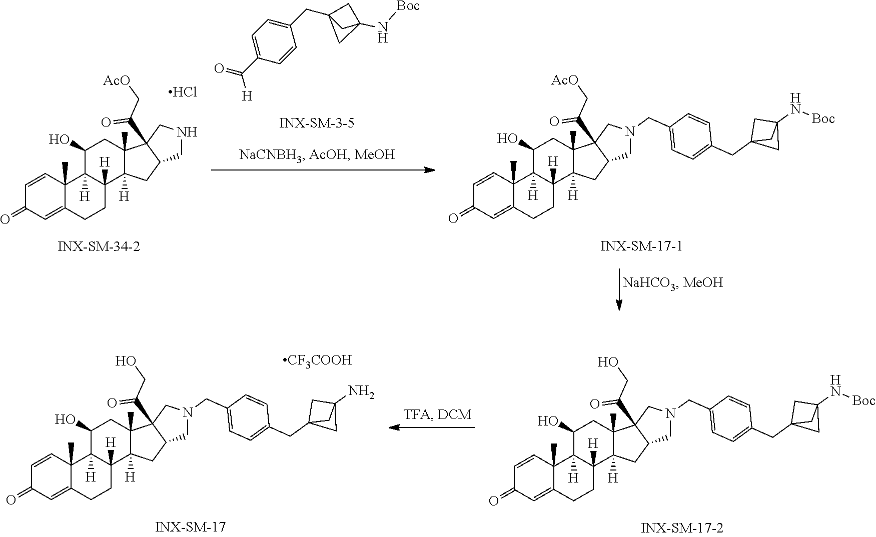

### Synthesis of 2-((6aR,6bS,7S,8aS,8bS,11aR,12aS,12bS)-10-(4-((3-((tert-butoxycarbonyl) amino)bicyclo[1.1.1]pentan-1-yl)methyl)benzyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetate carbamate (INX-SM-17-1)

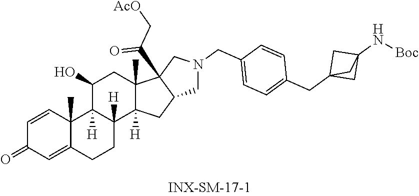

A 35 mL glass vial was charged with 2-((6aR,6bS,7S,8aS,8bS,11aR,12aS,12bS)-7-hydroxy-6a,8a-dimethyl-4-oxo-1,4,6a,6b,7,8,8a,9,10, 11,11a,12,12a,12b-tetradecahydronaphtho[2′,1′:4,5]indeno[1,2-c]pyrrol-8b(2H)-yl)-2-oxoethyl acetate (0.5 g, 1.17 mmol) (INX-SM-34-2) and tert-butyl (3-(4-formylbenzyl)bicyclo[1.1.1]pentan-1-yl)carbamate (0.352 g, 1.17 mmol) (INX-SM-3-5) in dioxane (5 mL). To this solution, AcOH (0.070 g, 1.16 mmol) and NaCNBH3 (0.088 g, 1.4 mmol) were added and stirred for 2 h at room temperature. After completion of reaction as indicated by TLC, reaction mixture was quenched with water and extracted with EtOAC. The combined organic layer was dried over Na₂SO₄ and evaporated under vacuum. The crude was purified by silica gel column chromatography (Hexane/ethyl acetate, 30:70) to give title compound as white solid (0.1 g, 12%). LCMS: 713.7 [M+H]+.

### Synthesis of tert-butyl (3-(4-(((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)methyl)benzyl)bicyclo[1.1.1]pentan-1-yl)carbamate (INX-SM-17-2)

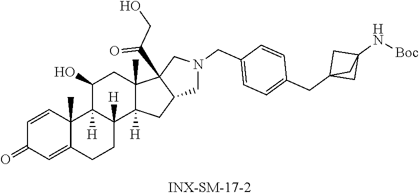

### Procedure

A 10 mL single-necked round bottom flask was charged with 2-((6aR,6bS,7S,8aS,8bS,11aR,12aS,12bS)-10-(4-((3-((tert-butoxycarbonyl)amino)bicyclo[1.1.1]pentan-1-yl)methyl)benzyl)-7-hydroxy-6a,8a-dimethyl-4-oxo-1,4,6a,6b,7,8,8a,9,10,11,11a,12,12a,12b-tetradecahydronaphtho[2′,1′:4,5]indeno[1,2-c]pyrrol-8b(2H)-yl)-2-oxoethyl acetatecarbamate (INX-SM-17-1) (0.1 g, 0.14 mmol) and methanol (2 mL). To this solution, NaHCO₃ (0.023 g, 0.28 mmol) was added and stirred at room temperature for 16 h. After completion of reaction as indicated by TLC, reaction mixture was diluted with ethyl acetate and filtered through celite. The combine organic layer was concentrated to give crude title compound product as off white solid (0.080 g, 85.01%). LCMS: 671.5 [M+H]+.

### Synthesis of (6aR,6bS,7S,8aS,8bS,11aR,12aS,12bS)-10-(4-((3-aminobicyclo[1.1.1]pentan-1-yl)methyl)benzyl)-7-hydroxy-8b-(2-hydroxyacetyl)-6a,8a-dimethyl-1,6a,6b,7,8,8a,8b,9,10,11,11a,12,12a,12b-tetradecahydronaphtho[2′,1′:4,5]indeno[1,2-c]pyrrol-4 (2H)-one 2,2,2-trifluoroacetate (INX-SM-17)

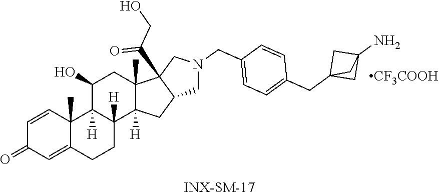

### Procedure

A 10 mL single-necked round bottom flash was charged with tert-butyl (3-(4-(((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)methyl)benzyl)bicyclo[1.1.1]pentan-1-yl)carbamate (INX-SM-17-2) (0.080 g) and DCM (2 mL). To this solution, TFA (1 mL) was added at room temperature and stirred for 1 h. After completion of reaction as indicated by TLC, reaction mixture was evaporated under vacuum. The crude was triturated with diethyl ether and n-pentane to give title compound as white solid (0.070 g, quantitative). LCMS: 571.4 [M+H]+; ¹H NMR (400 MHZ, MeOD): δ:7.47 (d, J=10.0 Hz, 1H), 7.42 (d, J=8.0 Hz, 2H), 7.25 (d, J=10.0 Hz, 2H), 6.28 (d, J=9.2 Hz, 1H), 6.05 (s, 1H), 4.48-4.26 (m, 5H), 3.87-3.77 (m, 2H), 3.60-3.20 (m, 2H), 2.96 (s, 2H), 2.90-2.60 (m, 2H), 2.50-2.00 (m, 4H), 1.89 (s, 6H), 1.70-1.55 (m, 2H), 1.51 (s, 3H), 1.30-1.19 (m, 3H), 1.07 (s, 3H)

### Synthesis of (S)-4-(2-(2-bromoacetamido)acetamido)-5-((3-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)amino)-5-oxopentanoic acid (INX-A29)

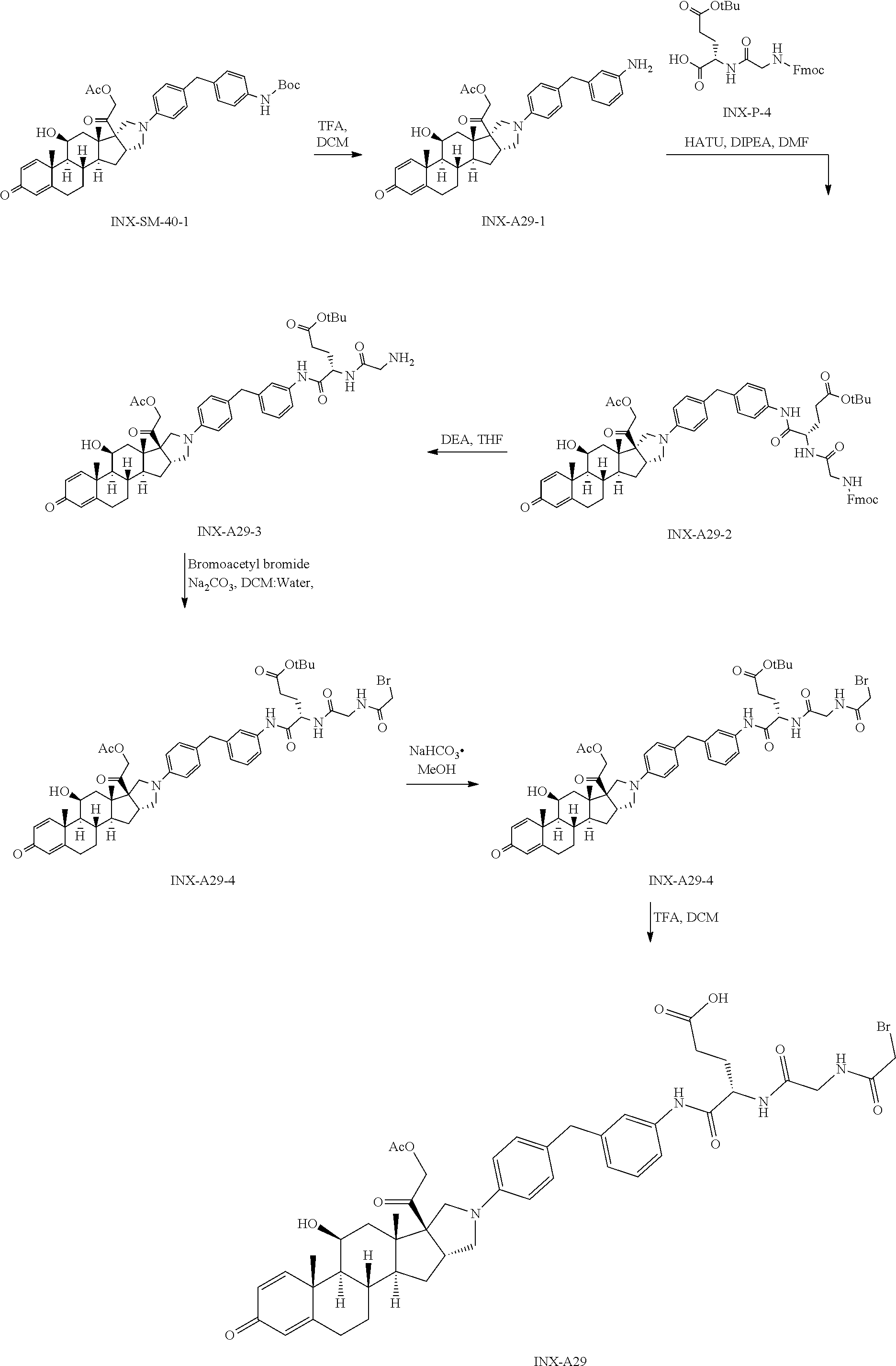

### Synthesis of 2-((6aR,6bS,7S,8aS,8bS,11aR,12aS,12bS)-10-(4-(3-aminobenzyl)phenyl)-7-hydroxy-6a,8a-dimethyl-4-oxo-1,4,6a,6b,7,8,8a,9,10,11,11a,12,12a,12b-tetradecahydronaphtho[2,1′: 4,5]indeno[1,2-c]pyrrol-8b(2H)-yl)-2-oxoethyl acetate (INX-29-A-1)

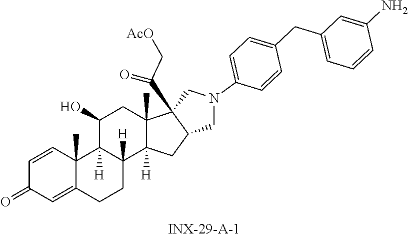

A 10 mL single-necked round bottom flash was charged with 2-((6aR,6bS,7S,8aS,8bS,11aR,12aS,12bS)-10-(4-(4-((tert-butoxycarbonyl)amino)benzyl)phenyl)-7-hydroxy-6a,8a-dimethyl-4-oxo-1,4,6a,6b,7,8,8a,9,10,11,11a,12,12a,12b-tetradecahydro naphtho[2′,1′:4,5]indeno[1,2-c]pyrrol-8b(2H)-yl)-2-oxoethyl acetate (INX-SM-40-1) (0.38 g, 0.53 mmol) and DCM (1 mL). To this solution, TFA (1.2 mL) was added and stirred for 1 h at room temperature. After completion of reaction as indicated by TLC, reaction mixture was evaporated under vacuum and triturated to give title compound as off white solid (0.22 g, 67.42%). LCMS: 609.4 [M+H]+.

### Synthesis of tert-butyl(S)-4-(2-((((9H-fluoren-9-yl)methoxy)carbonyl)amino)acetamido)-5-((4-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)amino)-5-oxopentanoate (INX-29-A-2)

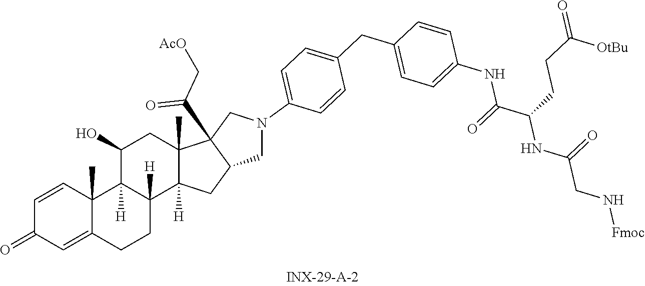

### Procedure

A 10 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) (0.39 g, 0.85 mmol), 2-((6aR,6bS,7S,8aS,8bS,11aR,12aS,12bS)-10-(4-(3-aminobenzyl)phenyl)-7-hydroxy-6a,8a-dimethyl-4-oxo-1,4,6a,6b,7,8,8a,9, 10, 11, 11a,12, 12a,12b-tetradecahydronaphtho[2′,1′:4,5]indeno[1,2-c]pyrrol-8b(2H)-yl)-2-oxoethyl acetate (INX-29A-1) (0.5 g, 0.85 mmol) and DMF (2 mL). To this solution, DIPEA (0.26 g, 2.05 mmol) and HATU (0.37 g, 0.98 mmol) were added and stirred for 15 min at room temperature. After completion of reaction as indicated by TLC, reaction mixture was poured into water and extracted with ethyl acetate. The combined organic layer was dried over Na₂SO₄ and evaporated under vacuum. The crude was triturated with diethyl ether to give title compound as off white solid (0.26 g, 29.47%). LCMS 1073.9 [M+H]+.

### Synthesis of tert-butyl(S)-5-((3-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)amino)-4-(2-aminoacetamido)-5-oxopentanoate (INX-29-A-3)

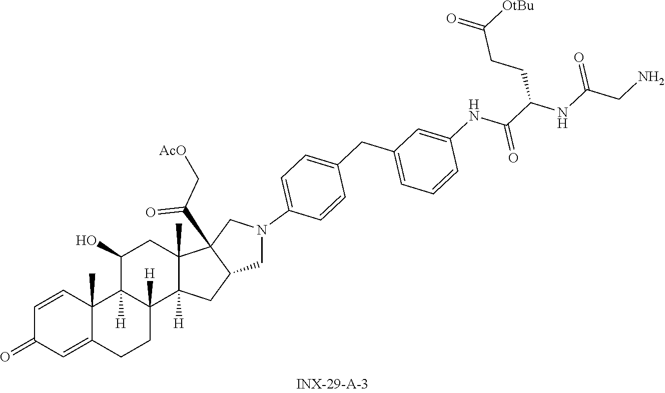

### Procedure

A 10 mL single-necked round bottom flash was charged with tert-butyl(S)-4-(2-((((9H-fluoren-9-yl)methoxy)carbonyl)amino)acetamido)-5-((4-(4-((6aR,6bS,7S,8aS,8bS,11aR, 12aS,12bS)-8b-(2-acetoxyacetyl)-7-hydroxy-6a,8a-dimethyl-4-oxo-1,4,6a,6b,7,8,8a,8b, 9,11,11a,12,12a,12b-tetradecahydronaphtho[2′,1′:4,5]indeno[1,2-c]pyrrol-10(2H)-yl) benzyl)phenyl)amino)-5-oxopentanoate (INX-29-A-2) (0.26 g, 0.24 mmol) and THF (5 mL). To this solution, diethyl amine (0.17 g, 2.4 mmol) was added and stirred for 2 h. After completion of reaction as indicated by TLC, reaction mixture was evaporated under vacuum and triturated with diethyl ether and pentane to give title compound as off white solid (0.14 g, 67.89%). LCMS: 851 [M+H]+.

### Synthesis of tert-butyl(S)-5-((3-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)amino)-4-(2-(2-bromoacetamido)acetamido)-5-oxopentanoate (INX-29-A-4)

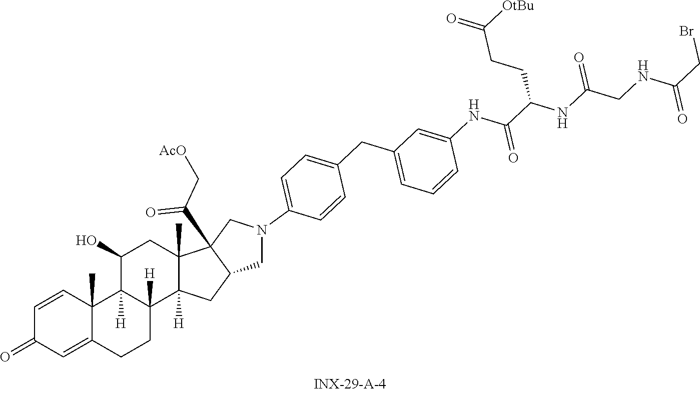

### Procedure

A 10 mL single-necked round bottom flash was charged with tert-butyl(S)-5-((3-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)amino)-4-(2-aminoacetamido)-5-oxopentanoate (INX-29-A-3) (0.14 g, 0.16 mmol) and DCM (2 mL). To this solution, NaHCO₃ (0.04 g, 0.49 mmol) solution in water (1 mL) and bromoacetyl bromide (0.05 g, 0.24 mmol) were added drop wise at room temperature and stirred for 1 h. After completion of reaction as indicated by TLC, reaction mixture was quenched with water and extracted with DCM. The combined organic layer was dried over Na₂SO₄ and evaporated under vacuum to give title compound as off white solid (0.18 g, quantitative). LCMS: calculated for C51H6479BrFN4O10 (971.38), found 971.80 [M+H]+.

### Synthesis of tert-butyl(S)-4-(2-(2-bromoacetamido)acetamido)-5-((3-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)amino)-5-oxopentanoate (INX-29-A-5)

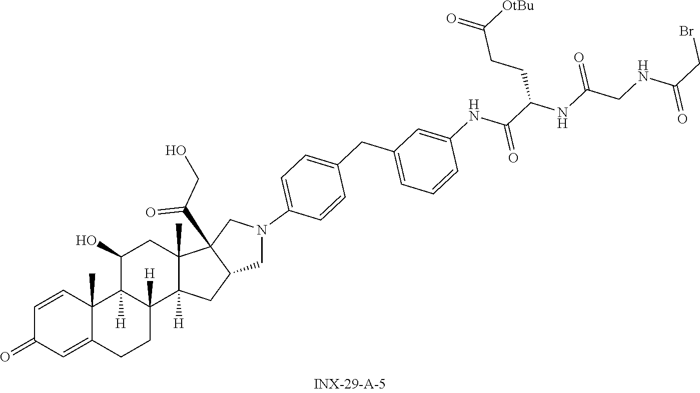

### Procedure

A 10 mL single-necked round bottom flask was charged with tert-butyl(S)-5-((3-(4-((6aR,6bS,7S,8aS,8bS,11aR,12aS,12bS)-8b-(2-acetoxyacetyl)-7-hydroxy-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)amino)-4-(2-(2-bromoacetamido)acetamido)-5-oxopentanoate (INX-29-A-4) (0.18 g, 0.18 mmol) and Methanol: H₂O (9:1, 2 mL). To this solution, NaHCO₃ (0.03 g, 0.37 mmol) was added and stirred at room temperature for 6 h. After completion of reaction as indicated by TLC, reaction mixture was diluted with ethyl acetate and filtered through celite bed. The combined organic layer was concentrated to give crude title compound product as off white solid (0.17 g 98.71%). LCMS: calculated for C49H6279BrFN4O9 (929.37), found 929.70 [M+H]+.

### Synthesis of (S)-4-(2-(2-bromoacetamido)acetamido)-5-((3-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12,12a,12b-tetradecahydronaphtho[2′,1′:4,5]indeno[1,2-c]pyrrol-10(2H)-yl)benzyl)phenyl)amino)-5-oxopentanoic acid (INX-29-A)

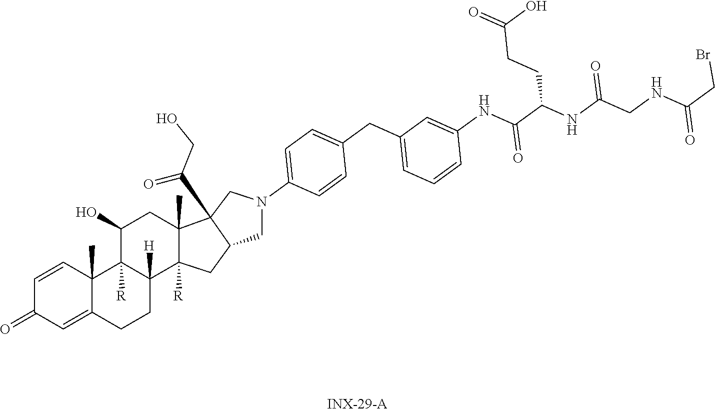

### Procedure

A 10 mL single-necked round bottom flash was charged with tert-butyl(S)-4-(2-(2-bromoacetamido)acetamido)-5-((3-(4-((6aR,6bS,7S,8aS,8bS,11aR,12aS,12bS)-7-hydroxy-8b-(2-hydroxyacetyl)-6a,8a-dimethyl-4-oxo-1,4,6a,6b,7,8,8a,8b,9,11,11a,12, 12a,12b-tetradecahydronaphtho[2′,1′:4,5]indeno[1,2-c]pyrrol-10(2H)-yl)benzyl)phenyl) amino)-5-oxopentanoate (INX-29-A-5) (0.17 g, 0.18 mmol) and DCM (1 mL). To this solution, TFA (0.85 mL) was added at room temperature and stirred for 2 h at room temperature. After completion of reaction as indicated by TLC, reaction mixture was evaporated under vacuum. The crude was purified by prep HPLC (YMC-Actus Triart Prep C18-S, 250×20 mm S-5 μm, 12 nm, Mobile phase: A=0.05% TFA in water, B=CAN, A:B=55:45, retention time 14 min) to give title compound as white solid (0.025 g 15.65%). LCMS: calculated for C45H5479BrFN4O9 (873.31), found 873.20 [M+H]+; ¹H NMR (400 MHZ, MeOD): □: 7.47 (d, J=10.0 Hz, 1H), 7.42-7.37 (m, 2H), 7.20 (d, J=8.0 Hz, 1H), 7.03 (d, J=8.4 Hz, 2H), 6.93 (d, J=7.2 Hz, =1H), 6.64 (d, J=8.4 Hz, =2H), 6.26 (dd, J=10.0 & 1.6 Hz, 1H), 5.99 (s, 1H), 4.53-4.26 (m, 4H), 3.93-3.92 (m, 3H), 3.83 (s, 2H), 3.57-2.98 (m, 6H), 2.70-1.56 (m, 12H), 1.50 (s, 3H), 1.07 (m, 5H).

### Synthesis Scheme of (6aR,6bS,7S,8aS,8bS,11aR,12aS,12bS)-10-(4-((3-aminobicyclo[1.1.1]pentan-1-yl)methyl)benzoyl)-7-hydroxy-8b-(2-hydroxyacetyl)-6a,8a-dimethyl-1,6a,6b,7,8,8a,8b,9,10,11,11a,12,12a,12b-tetradecahydronaphtho[2′,1′:4,5]indeno[1,2-c]pyrrol-4 (2H)-one (INX-SM-16)

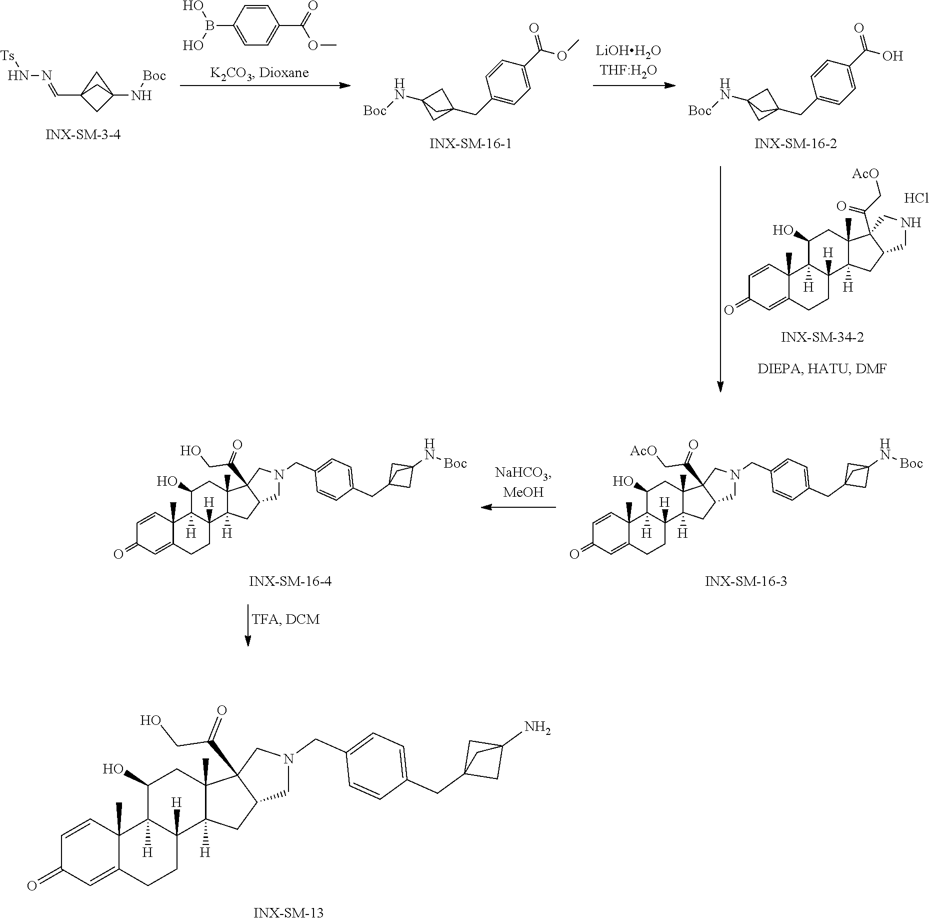

### Synthesis of methyl 4-((3-((tert-butoxycarbonyl)amino)bicyclo[1.1.1]pentan-1-yl)methyl)benzoate (INX-SM-16-1)

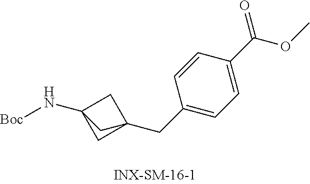 [SMILES: COC(=O)c1ccc(CC23CC(NC(=O)OC(C)(C)C)(C2)C3)cc1]

### Procedure

A 100 mL single-necked round bottom flask was charged with tert-butyl (E)-(3-((2-tosylhydrazono)methyl)bicyclo[1.1.1]pentan-1-yl)carbamate (INX-SM-3-4) (1.0 g, 2.64 mmol), dioxane (20 mL) and K2CO3 (0.576 g, 3.95 mmol) were added at degassed with N₂ (g) until white ppt observed. 4-(methoxycarbonyl)phenyl)boronic acid (0.75 g, 3.95 mmol) was added and allowed to stirred at 110° C. for 2 h. After completion of reaction as indicated by TLC, reaction mixture was poured into water and extracted with ethyl acetate. The combined organic layer was dried over Na2SO4 and evaporated under vacuum to give crude product. The crude was purified by silica gel column chromatography (ethyl acetate/hexane: 1:1) to give the title compound as colourless liquid (0.4 g, 45.80%). ¹H NMR (CDCl3) δ: 7.97 (d, J=8.4 Hz, 2H), 7.18 (d, J=8 Hz, 2H), 4.89 (s, 1H), 3.92 (s, 3H), 2.89 (s, 2H), 1.83 (s, 6H), 1.46 (s, 9H).

### Synthesis of 4-((3-((tert-butoxycarbonyl)amino)bicyclo[1.1.1]pentan-1-yl)methyl) benzoic acid (INX-SM-16-2)

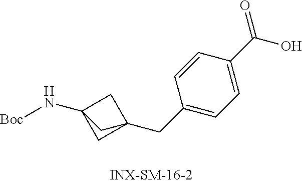 [SMILES: CC(C)(C)OC(=O)NC12CC(Cc3ccc(C(=O)O)cc3)(C1)C2]

### Procedure

A 100 mL single-necked round bottom flask was charged with methyl 4-((3-((tert-butoxycarbonyl)amino)bicyclo[1.1.1]pentan-1-yl)methyl)benzoate (INX-SM-16-1) (0.9 g, 2.72 mmol) and THF:H2O (1:1, 6 mL). To this solution, LiOH·H2O (0.65 g, 16.3 mmol) was added and allowed to stir at 50° C. for 5 h. After completion of reaction as indicated by TLC, reaction mixture was poured into water and extracted with 10% MeOH: DCM. The combined organic layer was dried over Na₂SO₄ and evaporated under vacuum to give title compound as white solid. (0.8 g, 92.82%). ¹H NMR (DMSO-d6) δ: 12.56 (brs, 1H) 7.85 (d, J=8.4 Hz, 2H), 7.41 (brs, 1H), 7.27 (d, J=8.4 Hz, 2H), 2.85 (s, 2H), 1.77 (s, 6H), 1.23 (s, 9H).

### Synthesis of 2-((6aR,6bS,7S,8aS,8bS,11aR,12aS,12bS)-10-(4-((3-((tert-butoxycarbonyl)amino)bicyclo[1.1.1]pentan-1-yl)methyl)benzoyl)-7-hydroxy-6a,8a-dimethyl-4-oxo-1,4,6a,6b,7,8,8a,9, 10, 11, 11a,12,12a,12b-tetradecahydronaphtho[2′,1′:4,5]indeno[1,2-c]pyrrol-8b(2H)-yl)-2-oxoethyl acetate (INX-SM-16-3)

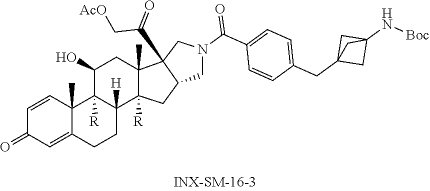

### Procedure

A 10 mL single-necked round bottom flash was charged with 4-((3-((tert-butoxycarbonyl)amino)bicyclo[1.1.1]pentan-1-yl)methyl)benzoic acid (INX-SM-16-2) (0.18 g, 0.56 mmol) and 2-((6aR,6bS,7S,8aS,8bS,11aR,12aS,12bS)-7-hydroxy-6a,8a-dimethyl-4-oxo-1, 6a,6b,8, 7, 8a,9, 10, 11, 11a,12,12a,12b-tetradecahydronaphtho[2′,1′:4,5]indeno[1,2-c]pyrrol-8b(2H)-yl)-2-oxoethyl acetate (INX-SM-34-2) (0.29 g, 0.68 mmol) in DMF (2 mL). To this solution DIPEA (0.8 g, 1.4 mmol) and HATU (0.32 g, 0.85 mmol) were added at room temperature and stirred for 15 min. After completion of reaction as indicated by TLC, reaction mixture was poured into water and extracted with ethyl acetate. The combined organic layer was dried over Na₂SO₄ and evaporated under vacuum. The crude was triturated with diethyl ether to give title compound as off white solid (0.19 g, 38.53%). The crude title compound was forwarded as such for the next step. LCMS: 727.43 [M+H]+.

### Synthesis of tert-butyl (3-(4-((6aR,6bS,7S,8aS,8bS,11aR,12aS,12bS)-7-hydroxy-8b-(2-hydroxyacetyl)-6a,8a-dimethyl-4-oxo-1, 2, 4, 6a,6b,7,8,8a,8b,9, 10, 11, 11a,12,12a, 12b-hexadecahydronaphtho[2′,1′:4,5]indeno[1,2-c]pyrrole-10-carbonyl)benzyl)bicyclo pentan-1-yl)carbamate (INX-SM-16-4)

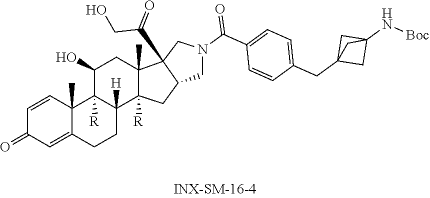

### Procedure

A 10 mL single-necked round bottom flask was charged with 2-((6aR,6bS,7S,8aS, 8bS,11aR,12aS,12bS)-10-(4-((3-((tert-butoxycarbonyl)amino)bicyclo[1.1.1]pentan-1-yl)methyl)benzoyl)-7-hydroxy-6a,8a-dimethyl-4-oxo-1,4,6a,6b,7,8,8a,9, 10, 11, 11a,12, 12a,12b-tetradecahydronaphtho[2′,1′:4,5]indeno[1,2-c]pyrrol-8b(2H)-yl)-2-oxoethyl acetate (INX-SM-16-3) (0.19 g, 0.26 mmol) and methanol (1 mL). To this solution, NaHCO₃ (0.04 g, 0.53 mmol) was added and allowed to stir at room temperature for 12 h. After completion of reaction as indicated by TLC, reaction mixture was diluted with ethyl acetate and filtered through celite bed. The combined organic layer was concentrated to give title compound crude product as off white solid (0.17 g, Crude). LCMS: 585.29 [M+H-Boc]+.

### Synthesis of (6aR,6bS,7S,8aS,8bS,12bS)-10-(4-((3-aminobicyclo[1.1.1]pentan-1-yl)methyl)benzoyl)-7-hydroxy-8b-(2-hydroxyacetyl)-6a,8a-dimethyl-1, 6a,6b,7,8,8a,8b,9, 10, 11, 11a,12,12a,12b-tetradecahydronaphtho[2′,1′:4,5]indeno[1,2-c]pyrrol-4 (2H)-one (INX-SM-16)

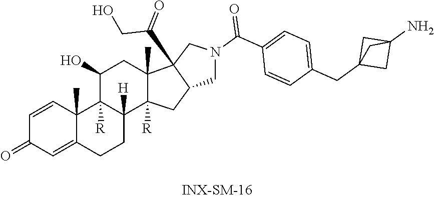

### Procedure

A 10 mL single-necked round bottom flask was charged with tert-butyl (3-(4-((6aR, 6bS,7S,8aS,8bS,11aR,12aS,12bS)-7-hydroxy-8b-(2-hydroxyacetyl)-6a,8a-dimethyl-4-oxo-1, 2, 4, 6a,6b,7,8,8a,8b,9, 10, 11, 11a,1 12a,12b-hexadecahydronaphtho[2′,1′:4,5]indeno[1,2-c]pyrrole-10-carbonyl)benzyl)bicyclo[1.1.1]pentan-1-yl)carbamate (INX-SM-16-4) (0.2 g, 0.29 mmol) and DCM (1 mL). To this solution, TFA (60.4 ml) was added and stirred for 2 h at room temperature. After completion of reaction as indicated by TLC, reaction mixture was evaporated under vacuum. The crude was purified by prep-HPLC (Column: NEW X-bridge Prep, C18, OBD (250×19) mm, 5 μm, Mobile phase: A=0.05% TFA in water, B=20% A line in Acetonitrile+5% Tetrahydrofuran, A:B=75:25) to give title compound (0.02 g 11.71%). LCMS: 585.50 [M+H]+; ¹H NMR (400 MHZ, MeOD): □: 7.50-7.39 (m, 3H), 7.23 (d, J=7.6 Hz, 2H), 7.30-7.27 (m, 1H), 6.04 (s, 1H), 4.57-3.54 (m, 7H), 2.97 (s, 2H), 2.80-2.00 (m, 6H), 1.90 (s, 6H), 1.89-1.60 (m, 2H), 1.50 (s, 3H), 1.45-1.12 (m, 4H), 1.10 (s, 3H).

### Synthesis of (S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)(methyl)amino)-5-oxopentanoic acid (INX-A21)

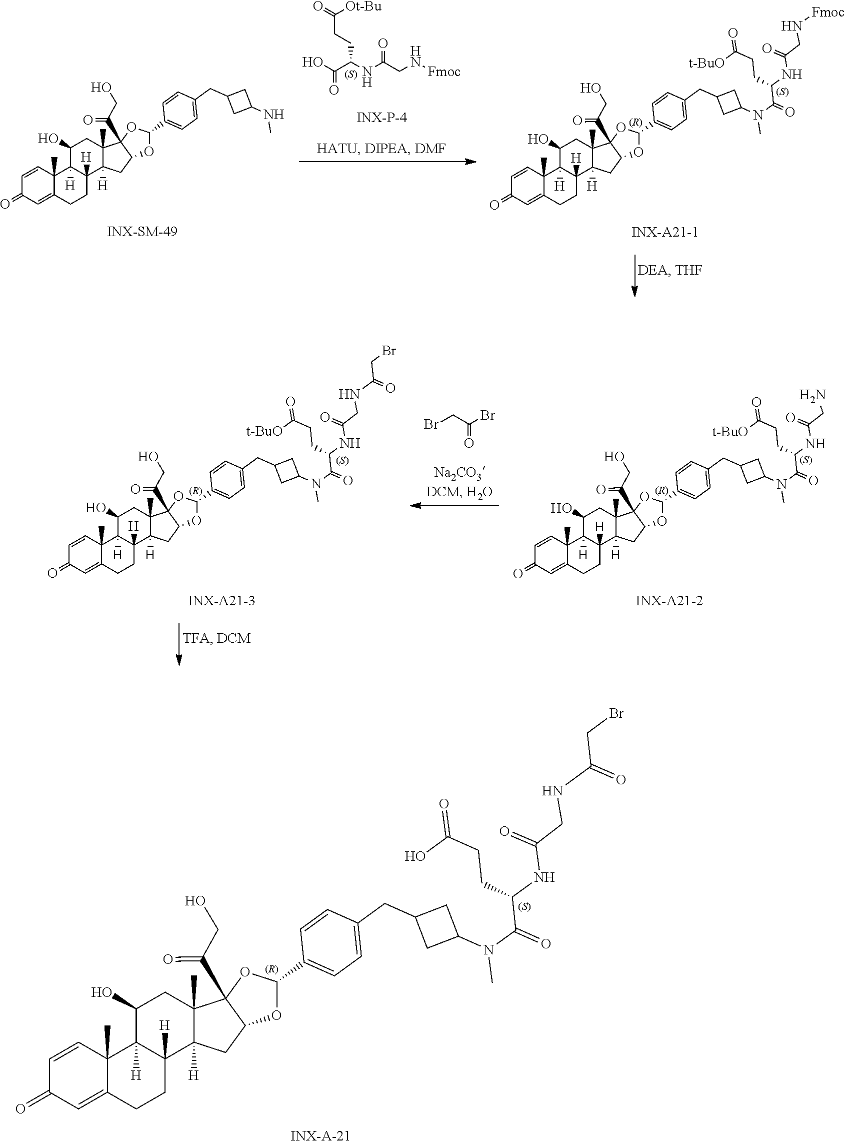

### Synthesis of tert-butyl(S)-4-(2-((((9H-fluoren-9-yl)methoxy)carbonyl)amino)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)(methyl)amino)-5-oxopentanoate (INX-A21-1)

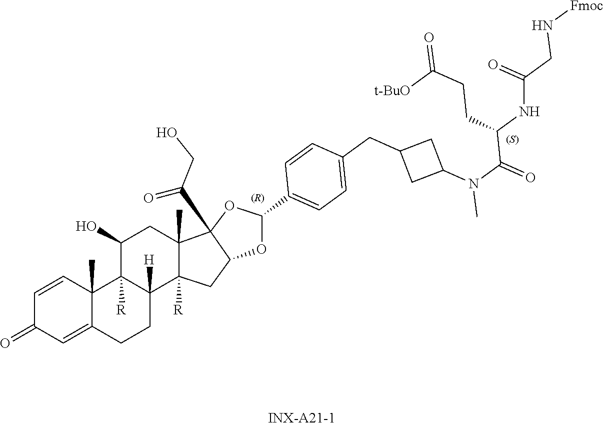

### Procedure

A 50 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) (0.34 g, 1.65 mmol), HATU (0.40, 1.05 mmol), DIPEA (0.18 g, 1.41 mmol) and DMF (3 mL) at room temperature. To this solution, (6aR,6bS,7S,8aS,8bS,10R,11aR,12aS,12bS)-7-hydroxy-8b-(2-hydroxyacetyl)-6a,8a-dimethyl-10-(4-((3-(methylamino) cyclobutyl)methyl)phenyl)-1,2,6a,6b,7,8,8a,8b,11a,12,12a,12b-dodecahydro-4H-naphtho[2′,1′:4,5]indeno[1,2-d][1,3]dioxol-4-one (INX-SM-49) (0.39 g, 0.7 mmol) was added and stirred for 1 h at room temperature. After completion of reaction as indicated by TLC, reaction mixture was poured into water and extracted with ethyl acetate. The combined organic layer was dried over Na2SO4 and evaporated under vacuum. The crude was purified by reverse phase column chromatography (acetonitrile/water: 60:40) to give title compound as light yellow solid (0.3 g, 42.10%). LCMS: 1026.92 [M+H]+.

### Synthesis of tert-butyl(S)-4-(2-aminoacetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)(methyl)amino)-5-oxopentanoate (INX-A21-2)

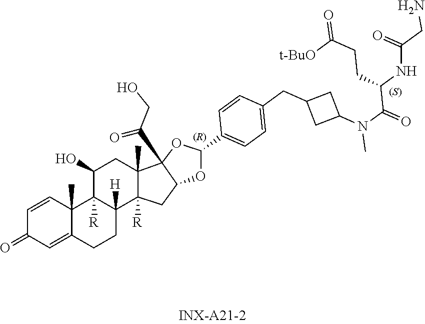

A 50 mL single-necked round bottom flash was charged with tert-butyl(S)-4-(2-((((9H-fluoren-9-yl)methoxy)carbonyl)amino)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)(methyl)amino)-5-oxopentanoate (INX-A-21-1) (0.3 g, 0.29 mmol) and THF (4100 mL). To this solution, diethylamine (0.21 g, 2.9 mmol) was added at room temperature and stirred for 3 h at room temperature. After completion of reaction as indicated by TLC, reaction mixture was evaporated under vacuum to give title compound as yellow solid (0.18 g, 76.59%) LCMS: 804.41 [M+H]+.

### Synthesis of tert-butyl (S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)(methyl)amino)-5-oxopentanoate (INX-A21-3)

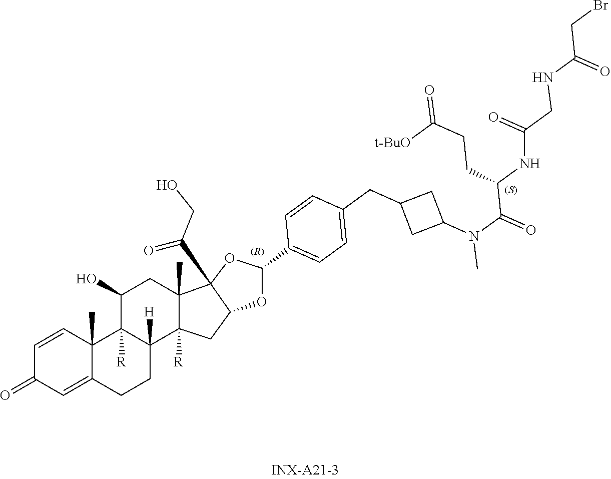

### Procedure

A 25 mL single-necked round bottom flash was charged with tert-butyl(S)-4-(2-aminoacetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)(methyl)amino)-5-oxopentanoate (INX-A21-2) (0.18 g, 0.22 mmol) in DCM (2 mL). To this solution, Na2CO3 (0.046 g, 0.44 mmol) solution in water (0.5 mL) followed by bromoacetyl bromide (0.067 g, 0.33 mmol) were added at room temperature and stirred for 1 h. After completion of reaction as indicated by TLC, reaction mixture was quenched with water and extracted with DCM. The combined organic layer was dried over Na₂SO₄ and evaporated under vacuum. The crude was triturated with diethyl ether to give title compound as pale yellow solid (0.18 g, 86.93%). LCMS: calculated for C47H6379BrFN3O11 (924.36), found 924.70 [M+H]+.

### Synthesis of (S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)(methyl)amino)-5-oxopentanoic acid (INX-A21)

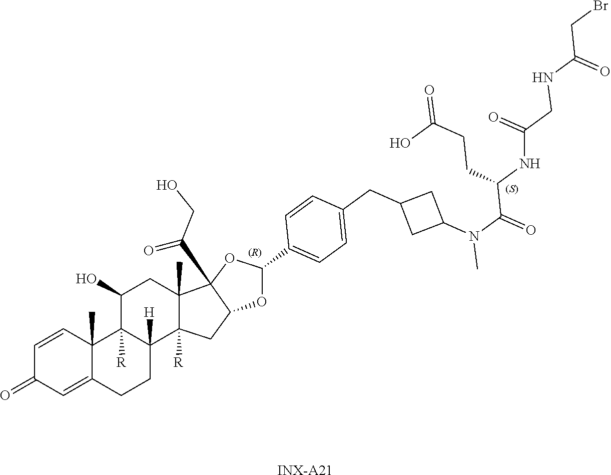

### Procedure

A 10 mL single-necked round bottom flash was charged with tert-butyl(S)-4-(2-(2-bromoacetamido)acetamido)-5-((3-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclobutyl)(methyl)amino)-5-oxopentanoate (INX-A21-3) (0.17 g, 0.18 mmol) in DCM (2 mL). To this solution, TFA (0.10 g, 0.91 mmol) was added and stirred for 2 h at room temperature. After completion of reaction as indicated by TLC, reaction mixture was evaporated under vacuum. The crude was purified by prep-HPLC to give title compound as off white solid (0.021 g, 13.15%); LCMS: calculated for C43H5579BrFN3O11 (868.30), found 868.40 [M+H]+; ¹H NMR (400 MHZ, MeOD): δ: 7.46 (d, J=10.0 Hz, 1H), 7.38-7.36 (m, 2H), 7.23-19 (m, 2H), 6.27 (dd, J=10.0 & 2.0 Hz, 1H), 6.04 (s, 1H), 5,46 (s, Acetal-H, 1H), 5.07 (s, J=5.2 Hz, C16-H, 1H), 5.04-5.00 (m, 1H), 4.46-4.15 (m, 4H), 3.95-3.89 (m, 4H), 3.30-1.60 (m, 23H), 1.52 (s, 3H), 1.08-1.05 (m, 2H) 1.00 (s, 3H).

### Synthesis of (6aR,6bS,7S,8aS,8bS,10R,11aR,12aS,12bS)-7-hydroxy-8b-(2-hydroxyacetyl)-6a,8a-dimethyl-10-(4-((octahydrocyclopenta[c]pyrrol-5-yl)methyl)phenyl)-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate) (INX-SM-48)

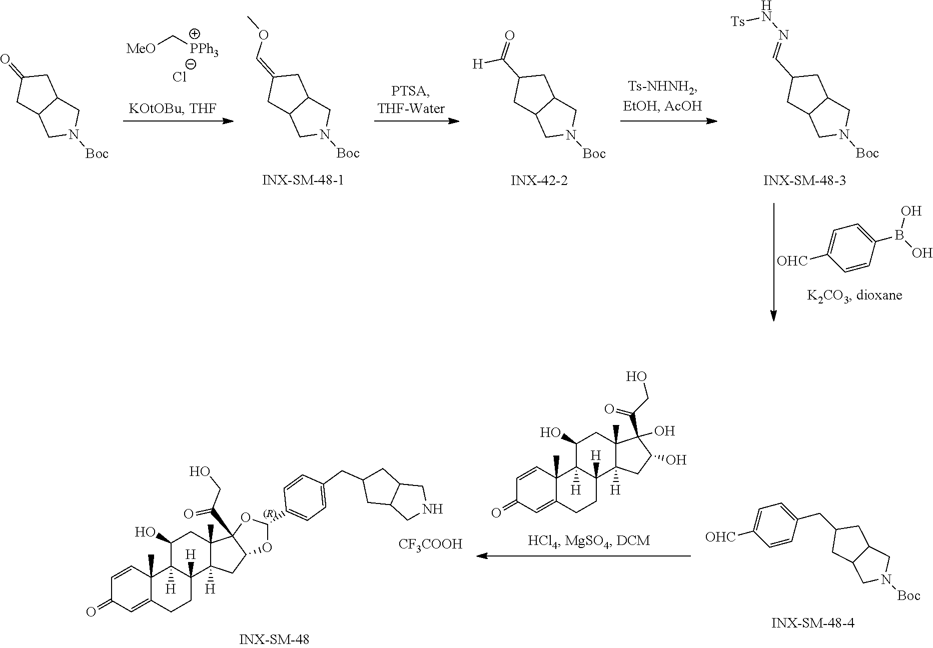

### Synthesis of tert-butyl 5-(methoxymethylene)hexahydrocyclopenta[c]pyrrole-2(1H)-carboxylate (INX-SM-48-1)

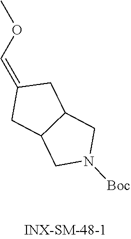 [SMILES: COC=C1CC2CN(C(=O)OC(C)(C)C)CC2C1]

### Procedure

A 100 mL single neck round bottom flask was charged with tert-butyl 5-oxohexahydrocyclopenta[c]pyrrole-2(1H)-carboxylate (1.0 g, 4.44 mmol), potassium tert-butoxide (0.99 g, 8.88 mmol) and THF (20 mL). To this solution, (Methoxymethyl) triphenylphosphonium chloride (2.74 g, 7.99 mmol) was added and stirred at room temperature for 1 h. After completion of reaction as indicated by TLC, reaction mixture was diluted with ammonium chloride solution and extracted with ethyl acetate. The combined organic layer was dried over Na₂SO₄ and evaporated under vacuum to give title compound as yellow solid (0.8 g, 71.14%), ¹H NMR (400 MHZ, DMSO-d6): δ: 5.98 (s, 1H), 3.48 (s, 3H), 3.41-3.37 (m, 2H), 2.98-2.90 (m, 2H), 2.59-2.53 (m, 2H), 2.41-2.33 (m, 2H), 2.04-1.96 (m, 2H), 1.38 (s, 9H).

### Synthesis of tert-butyl 5-formylhexahydrocyclopenta[c]pyrrole-2(1H)-carboxylate (INX-SM-48-2)

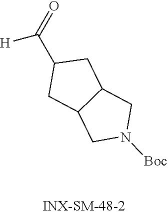 [SMILES: CC(C)(C)OC(=O)N1CC2CC(C=O)CC2C1]

### Procedure

A 100 mL single necked round bottom flask was charged with methyl tert-butyl 5-(methoxymethylene)hexahydrocyclopenta[c]pyrrole-2(1H)-carboxylate (INX-SM-48-1) (0.55 g, 2.17 mmol) in acetone (5 mL). To this solution, PTSA (0.41 g, 2.17 mmol) was added and stirred for 2 h at room temperature. After completion of reaction as indicated by TLC, reaction mixture was quenched with aqueous NaHCO₃ solution and extracted with dichloromethane. The combined organic layer was dried over Na₂SO₄ and evaporated under vacuum to give title compound as gummy solid (0.45 g, 95.28%). The crude was directly used for the next step without any analysis.

### Synthesis of tert-butyl (E)-5-((2-tosylhydrazono)methyl) hexahydrocyclopenta[c]pyrrole-2(1H)-carboxylate (INX-SM-48-3)

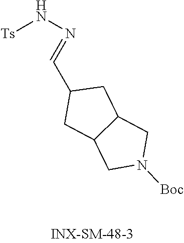 [SMILES: Cc1ccc(S(=O)(=O)N/N=C/C2CC3CN(C(=O)OC(C)(C)C)CC3C2)cc1]

### Procedure

A 35 mL glass vial was charged with tert-butyl 5-formylhexahydrocyclopenta[c]pyrrole-2 (1H)-carboxylate (INX-SM-48-2) (0.45 g, 1.88 mmol) and ethanol (5 mL). To this solution, p-toluenesulfonhydrazide (0.38 g, 2.06 mmol) and acetic acid (catalytic) were added and stirred for 1 h at room temperature. After completion of reaction as indicated by TLC, reaction mixture was poured into water and resulted white solid was filtered and dried under vacuum to give title compound (0.58 g 75.69%). LCMS [M+H]⁺ for C₂₀H₃₀N₃O₄S found to be 408.3.

### Synthesis of tert-butyl 5-(4-formylbenzyl)hexahydrocyclopenta[c]pyrrole-2(1H)-carboxylate (INX-SM-48-4)

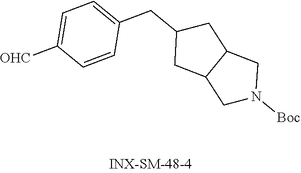 [SMILES: CC(C)(C)OC(=O)N1CC2CC(Cc3ccc(C=O)cc3)CC2C1]

### Procedure

A 50 mL single-necked round bottom flask was charged with tert-butyl (E)-5-((2-tosylhydrazono)methyl) hexahydrocyclopenta[c]pyrrole-2(1H)-carboxylate (INX-SM-48-3) (0.58 g, 1.44 mmol) and dioxane (10 mL). (4-formylphenyl)boronic acid (0.21 g, 1.44 mmol) and K2CO3 (0.29 g, 2.16 mmol) were added and stirred the reaction mixture for 2 h at 110° C. After completion of reaction as indicated by TLC, reaction mixture was poured into water and extracted with ethyl acetate. The combined organic layer was dried over Na₂SO₄ and evaporated under vacuum. The crude was purified by silica gel column chromatography (ethyl acetate/hexane, 50:50) to give title compound as colourless liquid (0.17 g, 35.8%). LCMS [(M+H)−^{t}Bu]⁺ for C₁₆H₂₀NO₃ found to be 273.8.

### Synthesis of (6aR,6bS,7S,8aS,8bS,10R,11aR,12aS,12bS)-7-hydroxy-8b-(2-hydroxyacetyl)-6a,8a-dimethyl-10-(4-((octahydrocyclopenta[c]pyrrol-5-yl)methyl)phenyl)-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-48)

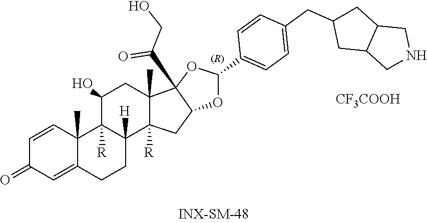

### Procedure

A 25 mL single-necked round bottom flask was charged with tert-butyl 5-(4-formylbenzyl)hexahydrocyclopenta[c]pyrrole-2(1H)-carboxylate (INX-SM-48-4) (0.16 g, 0.485 mmol) and (8S,9S,10R,11S,13S,14S,16R,17S)-11,16,17-trihydroxy-17-(2-hydroxyacetyl)-10,13-dimethyl-6,7,8,9,10,11,12,13,14,15,16, 17-dodecahydro-3H-cyclopenta[a]phenanthren-3-one (16-alfa-hydroxyprednisolone) (0.19 g, 0.51 mmol) in DCM (2 mL). To this solution, MgSO₄ (0.30 g, 2.55 mmol) and HClO₄ (0.25 g, 2.55 mmol) were added and stirred for 2 h at room temperature. After completion of reaction as indicated by TLC, reaction mixture was quenched with sat. NaHCO₃ solution and extracted with DCM. The combined organic layer was dried over Na₂SO₄ and evaporated under vacuum. The crude was purified by prep-HPLC (Column: YMC AQUA (250×19) mm, 5 μm, Mobile phase: A=0.05% TFA in water, B=Acetonitrile, A:B=66:34, retention time 8.78 min) to give title compound as white solid (0.040 g, 11.74%). LCMS [M+H]⁺ for C₃₆H₄₆NO₆ found to be 588.5; ¹H NMR (400 MHZ, DMSO-d6): δ: 8.76 (brs, 1H), 8.58 (brs, 1H), 7.37-7.31 (m, 3H), 7.22-7.16 (m, 2H), 6.17 (dd, J=1.6 Hz and 10.0 Hz, 1H), 5.94 (s, 1H), 5.41 (s, Acetal-H, 1H), 5.12-5.10 (m, 1H), 4.92 (d, J=4.8 Hz, C16-H, 1H), 4.82 (brs, 1H), 4.53-4-47 (m, 1H), 4.30 (brs, 1H), 4.21-4.17 (m, 1H), 3.20-3.00 (m, 2H), 2.80-1.51 (m, 20H), 1.38 (s, 3H), 1.07-0.96 (m, 2H), 0.87 (s, 3H).

### Synthesis of (4S)-4-(2-(2-bromoacetamido)acetamido)-5-(5-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)hexahydrocyclopenta[c]pyrrol-2 (1H)—I)-5-oxopentanoic acid (INX-A-20)

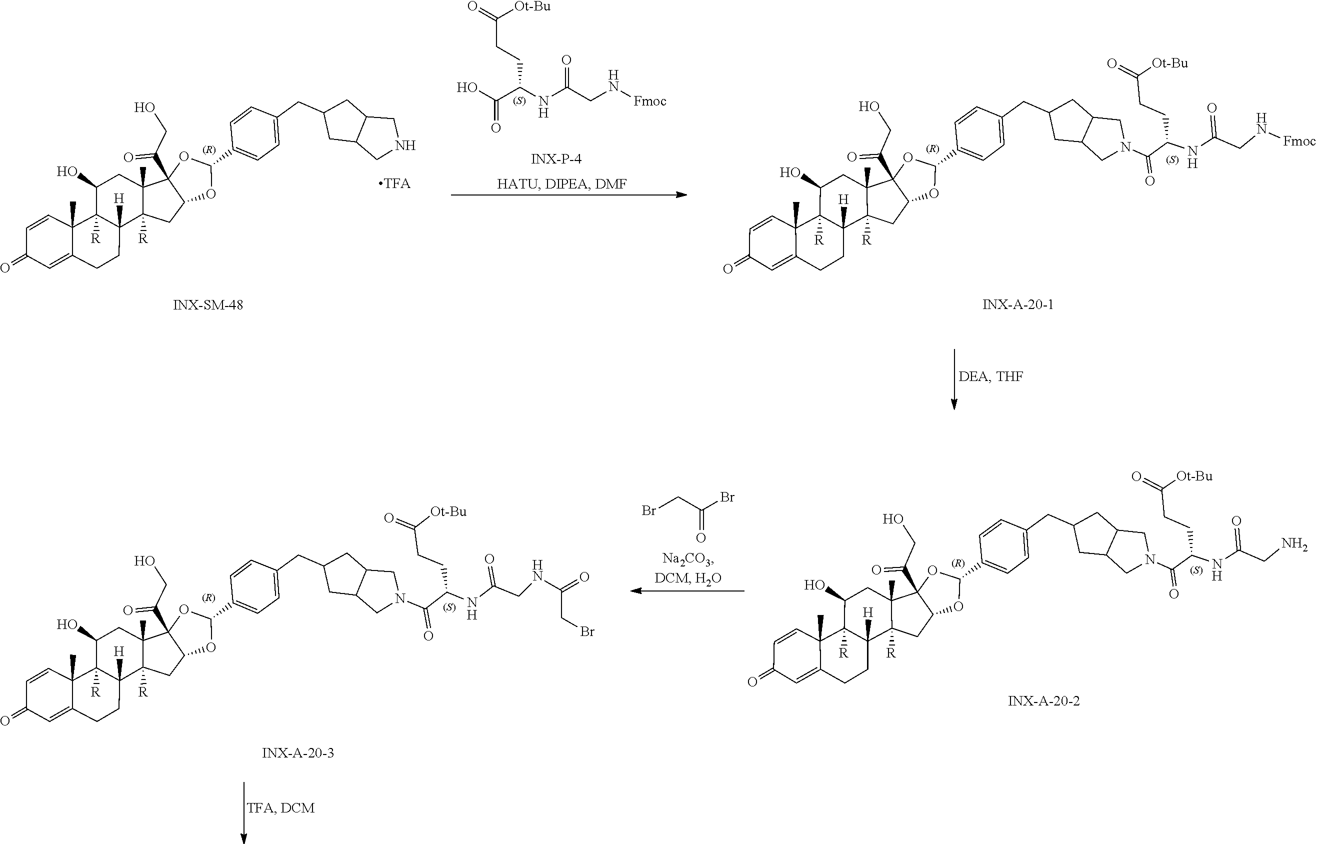

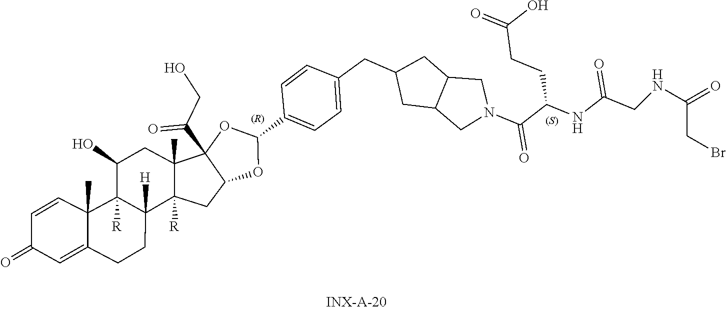

### Synthesis of tert-butyl (4S)-4-(2-((((9H-fluoren-9-yl)methoxy)carbonyl)amino)acetamido)-5-(5-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)hexahydrocyclopenta[c]pyrrol-2 (1H)-yl)-5-oxopentanoate (INX-A20-1)

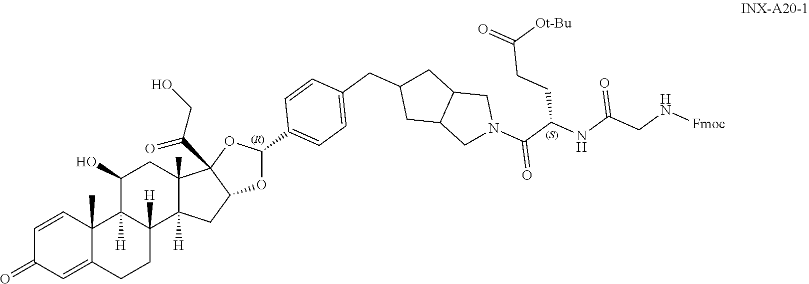

### Procedure

A 50 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) (0.53 g, 1. 10 mmol), HATU (0.62, 1.64 mmol), DIPEA (0.28 g, 2.19 mmol) and DMF (6 mL) at room temperature. To this solution, (6aR,6bS,7S,8aS,8bS,10R,11aR,12aS,12bS)-7-hydroxy-8b-(2-hydroxyacetyl)-6a,8a-dimethyl-10-(4-((octahydrocyclopenta[c]pyrrol-5-yl)methyl)phenyl)-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-48) (0.64 g, 0.91 mmol) was added and stirred for 1 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water: 60:40) to give title compound as pale yellow solid (0.32 g, 33.35%). LCMS: LCMS [M+H]⁺ for C₆₂H₇₄N₃O₁₂ found to be 1052.5.

### Synthesis of tert-butyl (4S)-4-(2-aminoacetamido)-5-(5-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)hexahydrocyclopenta[c]pyrrol-2 (1H)-yl)-5-oxopentanoate (INX-A20-2)

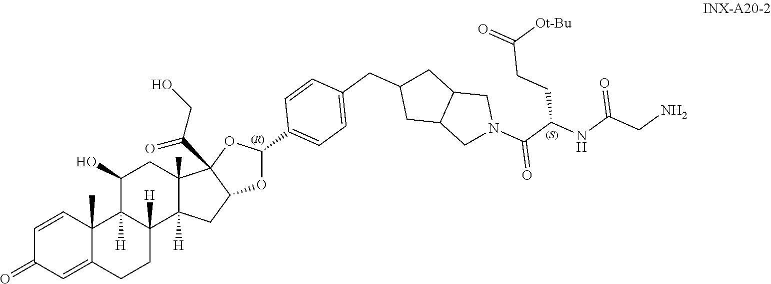

### Procedure

A 50 mL single-necked round bottom flash was charged with tert-butyl (4S)-4-(2-((((9H-fluoren-9-yl)methoxy)carbonyl)amino)acetamido)-5-(5-(4 ((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)hexahydrocyclopenta[c]pyrrol-2(1H)-yl)-5-oxopentanoate (INX-A20-1) (0.32 g, 0.30 mmol) and THF (3 mL). To this solution, diethylamine (0.22 g, 3.04 mmol) was added and stirred the reaction mixture for 3 h at room temperature. After completion of reaction as indicated by TLC, reaction mixture was evaporated under vacuum to give title compound as yellow solid (0.24 g, 96.4%). LCMS [M+H]⁺ for C₄₇H₆₄N₃O₁₀ found to be 830.5.

### Synthesis of tert-butyl (4S)-4-(2-(2-bromoacetamido)acetamido)-5-(5-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)hexahydrocyclopenta[c]pyrrol-2 (1H)-yl)-5-oxopentanoate (INX-A20-3)

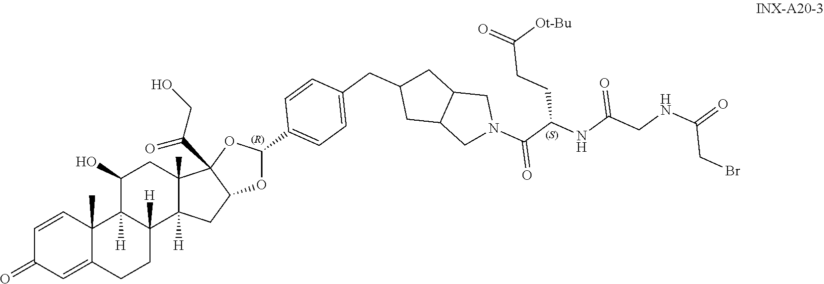

### Procedure

A 25 mL single-necked round bottom flash was charged with tert-butyl (4S)-4-(2-aminoacetamido)-5-(5-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)hexahydrocyclopenta[c]pyrrol-2(1H)-yl)-5-oxopentanoate (INX-A20-2) (0.24 g, 0.28 mmol) in DCM (2 mL). To this solution, Na₂CO₃ (0.058 g, 0.56 mmol) solution in water (0.5 mL) followed by bromoacetyl bromide (0.087 g, 0.43 mmol) were added and stirred for 1 h at room temperature. After completion of reaction as indicated by TLC, reaction mixture was quenched with water and extracted with DCM. The combined organic layer was dried over Na₂SO₄ and evaporated under vacuum. The crude was triturated with diethyl ether to give title compound as pale yellow solid (0.20 g, 75.1%). LCMS: calculated for C₄₉H₆₅⁷⁹BrN₃O₁₁ (950.38), found 950.40 [M+H]⁺.

### Synthesis of (4S)-4-(2-(2-bromoacetamido)acetamido)-5-(5-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a, 12,12a,12b-dodecahydro-1H-naphtho[2′,1′:4,5]indeno[1,2-d][1,3]dioxol-10-yl)benzyl)hexahydrocyclopenta[c]pyrrol-2 (1H)-yl)-5-oxopentanoic acid (INX-A20)

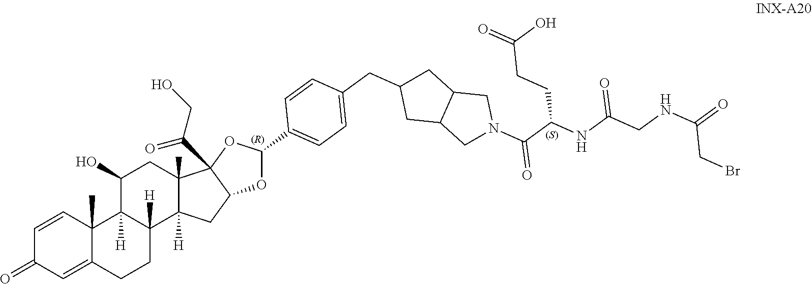

### Procedure

A 10 mL single-necked round bottom flash was charged with tert-butyl (4S)-4-(2-(2-bromoacetamido)acetamido)-5-(5-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)hexahydrocyclopenta[c]pyrrol-2(1H)-yl)-5-oxopentanoate (INX-A20-3) (0.17 g, 0.18 mmol) and DCM (2 mL). To this solution, TFA (0.10 g, 0.91 mmol) was added and stirred for 2 h at room temperature. After completion of reaction as indicated by TLC, reaction mixture was evaporated under vacuum. The crude title compound was purified by prep-HPLC (new Xbridge Prep, C18, OBD 19×250 mm, 5 μm, Mobile phase: A=0.05% TFA in water, B=Acetonitrile, A:B=90:10) to give two isomers of the product.

Fr-1 (retention time: 18 min) (0.0092 g, 5.75%, white solid) LCMS: calculated for C₄₅H₅₇⁷⁹BrN₃O₁₁ (894.32), found 894.5 [M+H]⁺; ¹H NMR (400 MHZ, MeOD): 0:7.46 (d, J=10.0 Hz, 1H), 7.35 (d, J=7.6 Hz, 2H), 7.19 (d, J=8.0 Hz, 2H), 6.26 (d, J=10 Hz, 1H), 6.04 (s, 1H), 5.45 (s, 1H, Acetal-H), 5.05 (d, J=5.2 Hz, 1H), 4.76-4.44 (m, 4H), 3.96-3.92 (m, 4H), 3.80-3.40 (m, 4H), 2.80-1.60 (m, 20H), 1.51 (s, 3H), 1.31-1.05 (m, 4H), 1.00 (s, 3H).

Fr-2 (retention time: 19.5 min) (0.0035 g, 2.19%, white solid). LCMS: calculated for C₄₅H₅₇⁷⁹BrN₃O₁₁ (894.3), found 894.3 [M+H]⁺; ¹H NMR (400 MHZ, MeOD): δ: 7.47 (d, J=10.0 Hz, 1H), 7.36 (d, J=7.6 Hz, 2H), 7.21-7.20 (m, 2H), 6.27 (d, J=10 Hz, 1H), 6.05 (s, 1H), 5.44 (s, 1H, Acetal-H), 5.06 (d, J=5.2 Hz, 1H), 4.80-4.30 (m, 4H), 4.00-3.70 (m, 4H), 3.65-1.5 (m, 26H), 1.54 (s, 3H), 1.25-1.05 (m, 2H), 1.00 (s, 3H).

### Synthesis of (S)-4-(2-(2-bromoacetamido)acetamido)-5-(((1S,4R)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoic acid (INX-A-28)

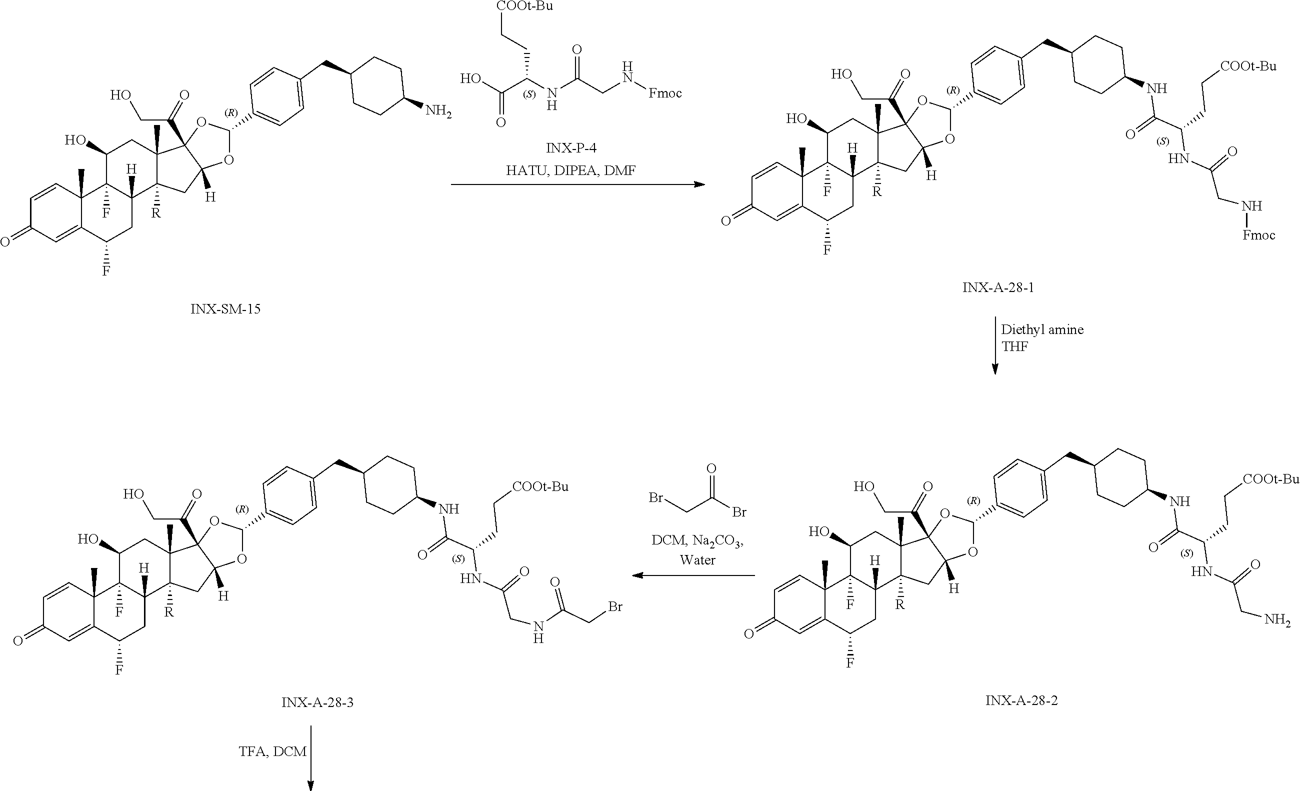

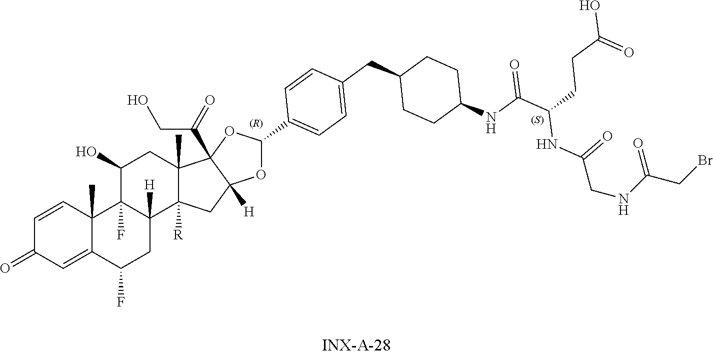

### Synthesis of tert-butyl(S)-4-(2-((((9H-fluoren-9-yl)methoxy)carbonyl)amino)acetamido)-5-(((1S,4R)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-28-1)

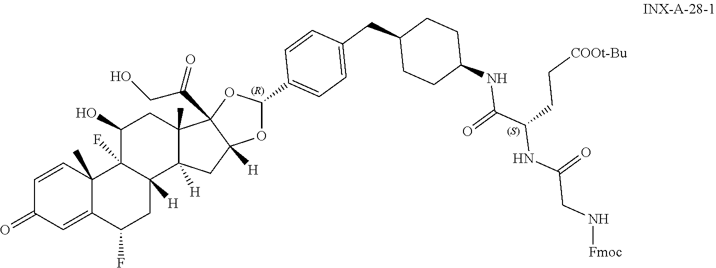

### Procedure

A 10 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) (0.39 g, 0.81 mmol) and HATU (0.37 g, 0.924 mmol), DIPEA (0.26 g, 0.82 mmol) and DMF (5 mL) at room temperature. To this solution, tert-butyl (2S,6aS,6bR,7S,8aS,8bS,10R,11aR, 12aS,12bS)-10-(4-(((1s,4S)-4-aminocyclohexyl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-15) (0.5 g, 0.81 mmol) was added and stirred for 1 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water: 50:50) to give title compound as light yellow solid (0.45 g, 41.8%). LCMS [M+H]⁺ for C₆₁H₇₂F₂N₃O₁₂ found to be 1077.0 [M+1]⁺.

### Synthesis of tert-butyl(S)-4-(2-aminoacetamido)-5-(((1S,4R)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-28-2)

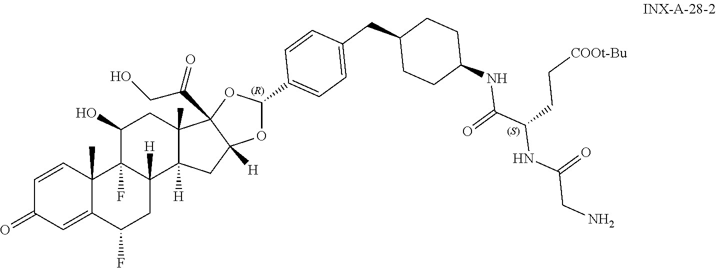

### Procedure

A 10 mL single-necked round bottom flash was charged with tert-butyl(S)-4-(2-((((9H-fluoren-9-yl)methoxy)carbonyl)amino)acetamido)-5-(((1S,4R)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-28-1) (0.4 g, 0.37 mmol) and THF (5 mL). To this solution, diethyl amine (0.27 g, 3.72 mmol) was added and stirred for 3 h at room temperature. After completion of reaction as indicated by TLC, reaction mixture was evaporated under vacuum and the crude was triturated with diethyl ether and pentane to give title compound as yellow solid (0.25 g, 79.1%). LCMS [M+H]⁺ for C₄₆H₆₂F₂N₃O₁₀ found to be 854.8.

### Synthesis of tert-butyl(S)-4-(2-(2-bromoacetamido)acetamido)-5-(((1S,4R)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-28-3)

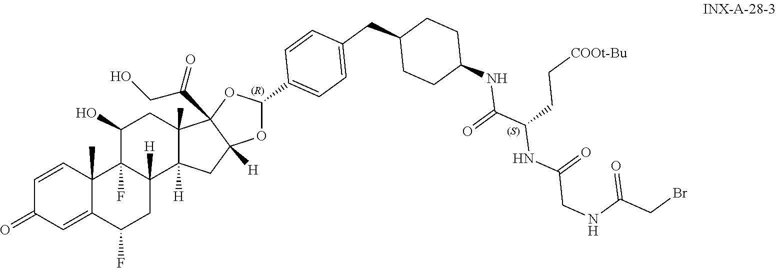

### Procedure

A 10 mL single-necked round bottom flash was charged with tert-butyl(S)-4-(2-aminoacetamido)-5-(((1S,4R)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-28-2) (0.25 g, 0.29 mmol) and DCM (4 mL). To this solution, Na₂CO₃ (0.12 g, 1.17 mmol) dissolved in water (0.5 mL) followed by bromoacetyl bromide (0.117 g, 0.58 mmol) were added to the reaction mixture and stirred at room temperature for 1 h. After completion of reaction as indicated by TLC, reaction mixture was quenched with water and extracted with DCM. The combined organic layer was dried over Na₂SO₄ and evaporated under vacuum. The crude was purified by reverse phase column chromatography (acetonitrile/water: 50:50) to give title compound pale yellow solid (0.2 g, 70.08%). LCMS: calculated for C₄₈H₆₃⁷⁹BrF₂N₃O₁₁ (974.36), found 974.4 [M+H]⁺.

### Synthesis(S)-4-(2-(2-bromoacetamido)acetamido)-5-(((1S,4R)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoic acid (INX-A-28)

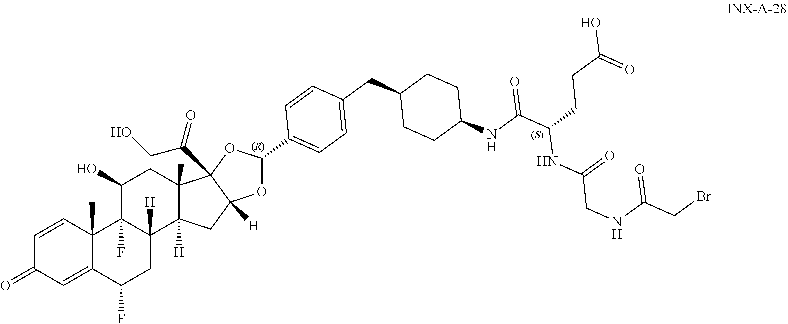

### Procedure

A 10 mL single-necked round bottom flash was charged with tert-butyl(S)-4-(2-(2-bromoacetamido)acetamido)-5-(((1S,4R)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-28-3) (0.2 g, 0.20 mmol) in DCM (4 mL). To this solution, TFA (0.4 mL) was added and stirred for 2 h at room temperature. After completion of reaction as indicated by TLC, reaction mixture was evaporated under vacuum. The crude was purified by prep-HPLC (Column: X-bridge Prep, C18, OBD (250×19) mm, 5 μm, Mobile phase: A=0.05% TFA in water, B=Acetonitrile, A:B=65:35, retention time 14.40 min) to give title compound as white solid (0.015 g, 8.16%). LCMS: calculated for C₄₄H₅₅⁷⁹BrF₂N₃O₁₁ (918.30), found 918.5 [M+H]⁺; ¹H NMR (400 MHZ, CD3OD): δ: 7.45-7.36 (m, 3H), 7.22-7.20 (m, 2H), 6.40-6.35 (m, 2H), 5.66-5.50 (m, 1H, CH—F), 5.48 (s, 1H, Acetal-H), 5.07 (d, J=4.4 Hz, 1H), 4.66 (d, 1H), 4.43-4.30 (m, 3H), 3.95 (s, 2H), 3.92 (s, 2H), 3.86 (brs, 1H), 2.80-1.65 (m, 17H), 1.55 (s, 3H), 1.50-1.36 (m, 6H), 1.01 (s, 3H).

### Synthesis of (6aR,6bS,7S,8aS,8bS,10R,11aR,12aS,12bS)-10-(4-((4-aminocyclohexyl)oxy) phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-18)

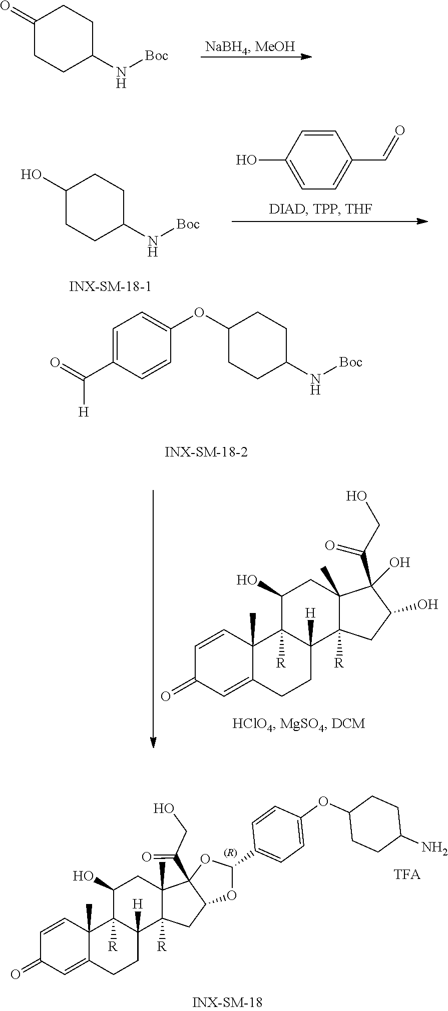

### Synthesis of tert-butyl (4-hydroxycyclohexyl)carbamate (INX-SM-18-1)

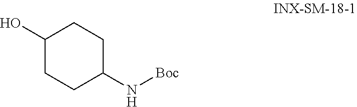 [SMILES: CC(C)(C)OC(=O)NC1CCC(O)CC1]

### Procedure

A 100 mL single-necked round bottom flash was charged with tert-butyl (4-oxocyclohexyl)carbamate (5.0 g, 23.43 mmol) and MeOH (50 mL) under nitrogen. To this solution NaBH₄ (7.43 g, 117.16 mmol) was added portion-wise at 0° C. and stirred for another 1 h at 0° C. After completion of reaction as indicated by TLC, reaction mixture was diluted with water and adjusted neutral pH with 1N HCl and extracted with ethyl acetate. The combined organic layer was dried over Na₂SO₄ and evaporated under vacuum to give title compound (4.8 g, 95.10%). LCMS [M+H]⁺ for C₁₁H₂₂NO₃ found to be 216.2.

### Synthesis of tert-butyl (4-(4-formylphenoxy) cyclohexyl)carbamate (INX-SM-18-2)

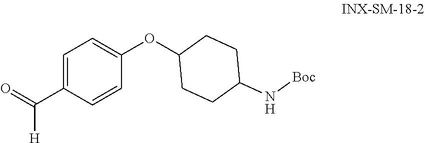 [SMILES: CC(C)(C)OC(=O)NC1CCC(Oc2ccc(C=O)cc2)CC1]

A 35 mL glass vial was charged with tert-butyl (4-hydroxycyclohexyl)carbamate (INX-SM-18-1) (1 g, 4.64 mmol), 4-hydroxybenzaldehyde (0.680 g, 5.57 mmol) and triphenylphosphine (1.82 g, 6.96 mmol) in THF (10 mL) under nitrogen. To this solution DIAD (1.4 g, 6.967 mmol) was added at 0° C. and reaction mixture was refluxed at 65° C. for 16 h. After completion of reaction as indicated by TLC, reaction mixture was diluted with 10% NaOH solution and extracted with ethyl acetate. The combined organic layer was dried over Na₂SO₄ and evaporated under vacuum. The crude was purified by column chromatography (ethyl acetate/hexane, 80:20) to give title compound as white solid (1.0 g, 67.40%). LCMS [M+H]⁺ for C₁₈H₂₆NO₄ found to be 320.2.

### Synthesis of (6aR,6bS,7S,8aS,8bS,10R,11aR,12aS,12bS)-10-(4-((4-aminocyclohexyl)oxy) phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one 2,2,2-trifluoroacetate (INX-SM-18)

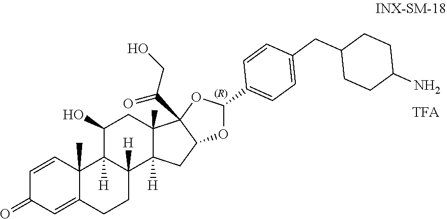

### Procedure

A 50 mL single-necked round bottom flash was charged with tert-butyl (4-(4-formylphenoxy) cyclohexyl)carbamate (INX-SM-18-2) (1 g, 3.13 mmol), (8S,9S,10R,11S,13S,14S,16R,17S)-11,16,17-trihydroxy-17-(2-hydroxyacetyl)-10,13-dimethyl-6,7,8,9,10,11,12,13, 14, 15, 16, 17-dodecahydro-3H-cyclopenta[a]phenanthren-3-one (16-alpha-hydroxyprednisolone) (1.17 g, 3.13 mmol) in DCM (10 mL) To this solution, MgSO₄ (1.57 g, 15.65 mmol) and HClO₄ (1.88 g, 15.65 mmol) were added and stirred for another 2 h at room temperature. After completion of reaction as indicated by TLC, reaction mixture was quenched with sat. NaHCO₃ solution and concentrated under vacuum. The crude was triturated with cold water to give 1.5 g of crude solid. A portion of crude (308 mg) was purified by prep-HPLC (Column: SHIM-PACK-GIST C18 (2508 (250×20) mm, 5 μm, Mobile phase: A=0.05% TFA in water, B=Acetonitrile, A:B=70:30, retention time 11.80 min) to give 0.019 g of title compound which would correspond to a total purified mass of 0.092 g (0.137 mmol, 4.38%) as a white solid. LCMS [M+H]⁺ for C₃₄H₄₃NO₇ found to be 578.4; ¹H NMR (400 MHZ, MeOD): δ: 7.47 (d, J=10.0 Hz, 1H), 7.40-7.36 (m, 2H), 6.98-6.92 (m, 2H), 6.27 (dd, J=1.6 Hz, J=10.0 Hz, 1H), 6.04 (s, 1H), 5.43 (m, 1H), 5.04 (d, J=5.2 Hz, 1H), 4.65 (d, 1H), 4.44 (brs, 1H), 4.36-4.30 (m, 2H), 3.20 (brs, 1H), 2.80-1.54 (m, 15H), 1.49 (s, 3H), 1.30-1.02 (m, 4H), 1.00 (s, 3H).

### Synthesis of S)-4-(2-(2-bromoacetamido)acetamido)-5-(((1R,4S)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoic acid (INX-A27)

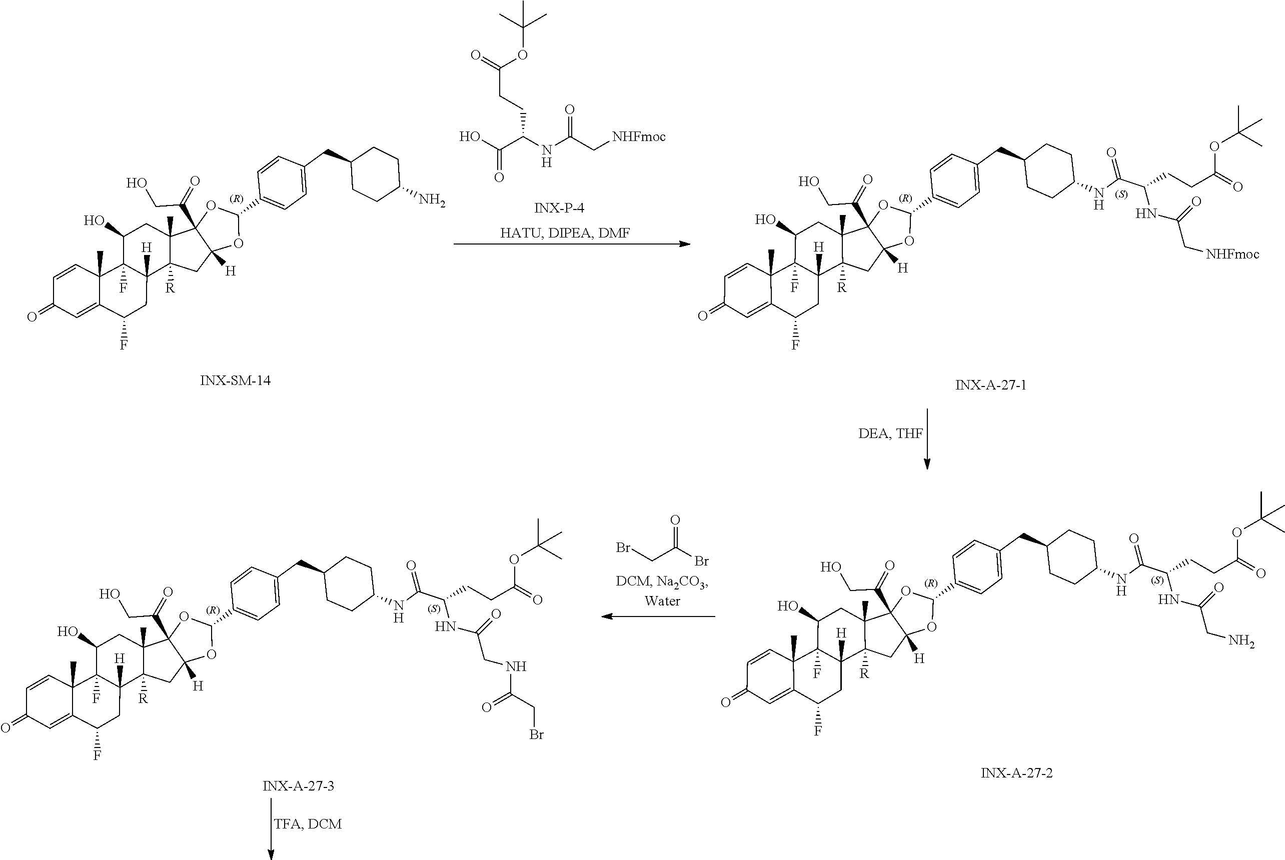

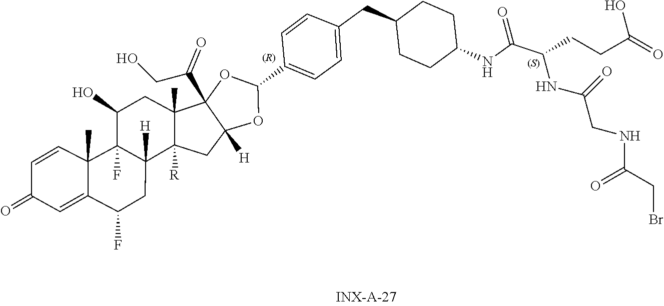

### Synthesis of tert-butyl(S)-4-(2-((((9H-fluoren-9-yl)methoxy)carbonyl)amino)acetamido)-5-(((1R,4S)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-27-1)

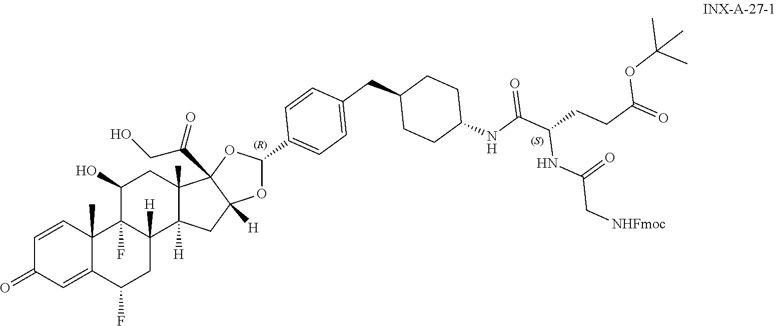

### Procedure

A 50 mL single-necked round bottom flash was charged with(S)-2-(2-((((9H-fluoren-9-yl)methoxy)carbonyl)amino)acetamido)-5-(tert-butoxy)-5-oxopentanoic acid (INX-P-4) (0.39 g, 0.81 mmol), HATU (0.46, 1.2 mmol), DIPEA (0.20 g, 1.62 mmol) and DMF (3 mL) at room temperature. To this solution, (2S,6aS,6bR,7S,8aS,8bS,10R,11aR,12aS,12bS)-10-(4-(((1r,4R)-4-aminocyclohexyl)methyl)phenyl)-2,6b-difluoro-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (INX-SM-14) (0.49 g, 0.81 mmol) was added and stirred for 1 h at room temperature. After completion of reaction as indicated by TLC, reaction mixture was poured into water and extracted with ethyl acetate. The combined organic layer was dried over Na₂SO₄ and evaporated under vacuum. The crude was purified by reverse phase column chromatography (acetonitrile/water: 60:40) to give title compound as off white solid (0.50 g, 57.48%). LCMS: 1076.7 [M+H]⁺

### Synthesis of tert-butyl(S)-4-(2-aminoacetamido)-5-(((1R,4S)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-27-2)

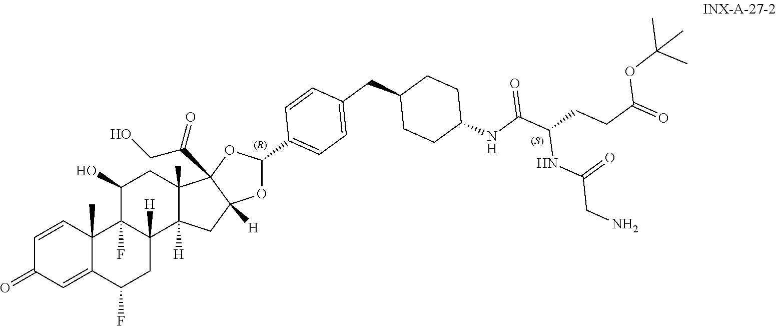

### Procedure

A 50 mL single-necked round bottom flash was charged with tert-butyl(S)-4-(2-((((9H-fluoren-9-yl)methoxy)carbonyl)amino)acetamido)-5-(((1R,4S)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-27-1) (0.5 g, 0.46 mmol) and THF (5 mL). To this solution, diethylamine (0.32 g, 4.6 mmol) was added and stirred for 3 h at room temperature. After completion of reaction as indicated by TLC, reaction mixture was evaporated under vacuum to give title compound as yellow solid (0.3 g, 75.61%) LCMS: 854.64 [M+H]⁺

### Synthesis of tert-butyl(S)-4-(2-(2-bromoacetamido)acetamido)-5-(((1R,4S)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-27-3)

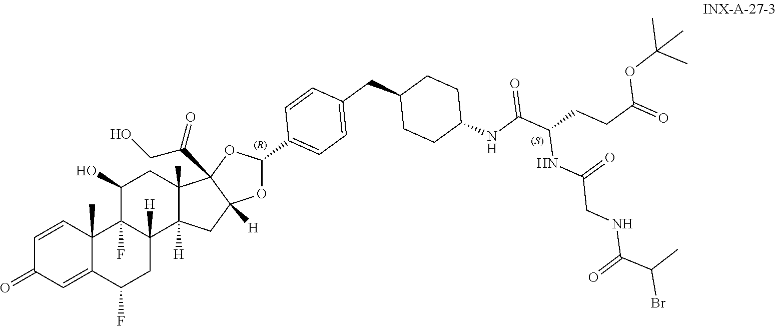

### Procedure

A 25 mL single-necked round bottom flash was charged with tert-butyl(S)-4-(2-aminoacetamido)-5-(((1R,4S)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-27-2) (0.3 g, 0.35 mmol) in DCM (2 mL). To this solution, Na₂CO₃ (0.073 g, 0.7 mmol) solution in water (0.5 mL) followed by bromoacetyl bromide (0.10 g, 0.52 mmol) were added at room temperature and stirred for 1 h. After completion of reaction as indicated by TLC, reaction mixture was quenched with water and extracted with DCM. The combined organic layer was dried over Na₂SO₄ and evaporated under vacuum. The crude was triturated with diethyl ether to give title compound as pale yellow solid (0.30 g, 87.60%). LCMS: calculated for C₄₈H₆₃⁷⁹BrF₂N₃O₁₁ (974.36), found 974.69 [M+H]⁺.

### Synthesis of (S)-4-(2-(2-bromoacetamido)acetamido)-5-(((1R,4S)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoic acid (INX-A-27)

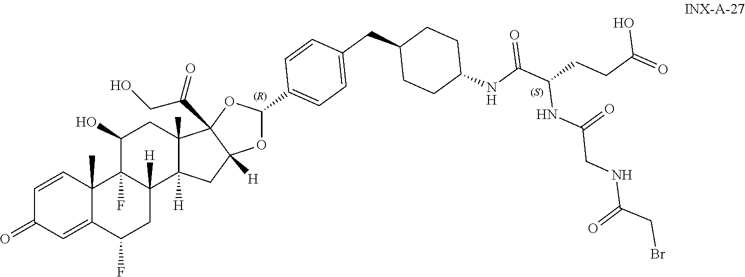

### Procedure

A 10 mL single-necked round bottom flash was charged with tert-butyl(S)-4-(2-(2-bromoacetamido)acetamido)-5-(((1R,4S)-4-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl)cyclohexyl)amino)-5-oxopentanoate (INX-A-27-3) (0.15 g, 0.15 mmol) in DCM (2 mL). To this solution, TFA (0.087 g, 0.76 mmol) was added and stirred for 2 h at room temperature. After completion of reaction as indicated by TLC, reaction mixture was evaporated under vacuum. The crude was purified by prep-HPLC (Column: YMC-Actus Triart Prep C18-S, 250×20 mm S-10 μm, 12 nm, mobile phase: A=0.05% TFA in water, B=Acetonitrile, A:B=60:40, retention time 16.00 min) to give title compound as white solid (0.010 g, 7.07%), LCMS: calculated for C₄₄H₅₅⁷⁹BrF₂N₃O₁₁ (918.30), found 918.20 [M+H]⁺; ¹H NMR (400 MHZ, MeOD): δ: 7.37-7.33 (m, 3H), 7.17 (d, J=8.0 Hz, 2H), 6.38-6.35 (m, 2H), 5.66-5.50 (m, 1H, CH—F), 5.48 (s, 1H), 5.05 (d, J=4.8 Hz, 1H), 4.65 (d, 1H), 4.37-4.31 (m, 3H), 3.94 (s, 2H), 3.90 (s, 2H), 3.60-3.50 (m, 1H), 2.80-1.66 (m, 18H), 1.62 (s, 3H), 1.50-1.04 (m, 5H), 1.00 (s, 3H)

### Synthesis of 6-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-aminium 2,2,2-trifluoroacetate (INX-SM-38)

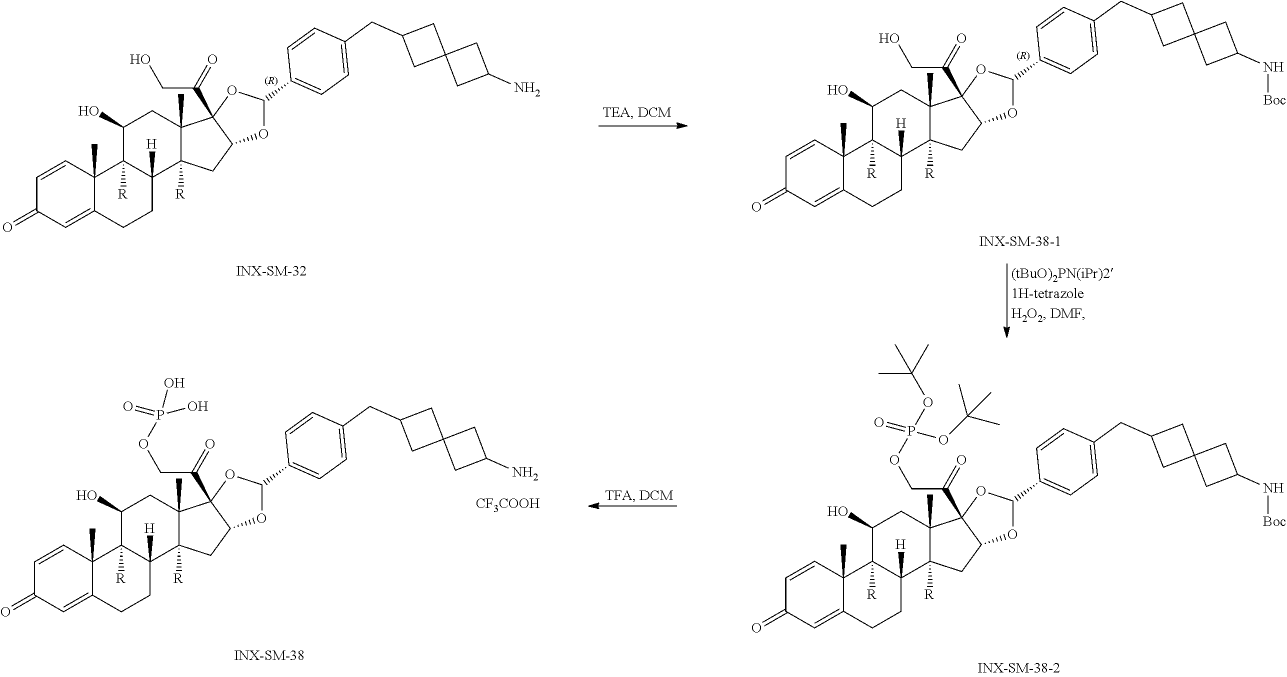

### Synthesis of tert-butyl (6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)carbamate (INX-SM-38-1)

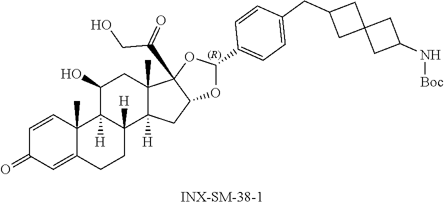

A 100 mL single necked round bottom flask was charged with (6aR,6bS,7S,8aS,8bS,10R,11aR,12aS,12bS)-10-(4-((6-aminospiro[3.3]heptan-2-yl)methyl)phenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (1.0 g, 1.70 mmol) (INX-SM-32), TEA (0.47 mL, 3.40 mmol) and DCM (10 mL)-MeOH (0.5 mL) at room temperature. To this solution, di-tert-butyldicarbonate (0.74 g, 3.40 mmol) was added and stirred the reaction mixture for 2 h at room temperature. After completion of reaction as indicated by TLC, reaction mixture was direct evaporated under vacuum. The crude was purified by reverse phase column chromatography (acetonitrile:water, 70:30) to give title compound as pale yellow solid (0.25 g, 21.36%). LCMS [M+H]⁺ for C₄₁H₅₄NO₈ found to be 688.5 [M+H]⁺.

### Synthesis of tert-butyl (6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)carbamate (INX-SM-38-2)

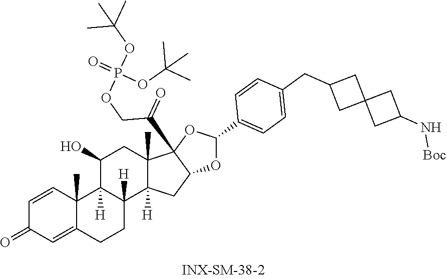

### Procedure

A 35 mL glass vial was charged with tert-butyl (6-(4-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)carbamate (0.25 g, 0.36 mmol) (INX-SM-38-1) in DMF (0.5 mL). To this solution, 1H-tetrazole (0.254 g, 3.63 mmol) and (tBuO)₂PNEt₂ (2.42 g, 8.73 mmol) and stirred at room temperature for 24h. Hydrogen peroxide (2.5 mL) was added to the reaction mixture at −5 to 0° C. and allowed to stir at room temperature for 1 h. The crude was purified by reverse phase column chromatography (acetonitrile:water, 90:10) to give title compound as off-white solid (0.18 g, 56.81%). LCMS [M+H]⁺ for C₄₉H₇₁NO₁₁P found to be 880.6 [M+H]⁺.

### Synthesis of 6-(4-((6aR,6bS,7S,8aS,8bS,10R,11aR,12aS,12bS)-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-aminium 2,2,2-trifluoroacetate (INX-SM-38)

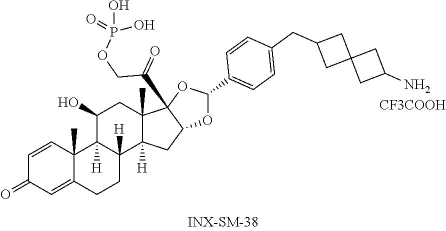

### Procedure

A 25 ml single-necked round bottom flash was charged with tert-butyl (6-(4-((6aR,6bS,7S,8aS,8bS,10R,11aR,12aS,12bS)-8b-(2-((di-tert-butoxyphosphoryl)oxy)acetyl)-7-hydroxy-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)carbamate (0.17 g, 0.193 mmol) (INX-SM-38-2) in DCM (4 mL). To this solution, TFA (0.17 mL) was added at 0° C. and stirred further for 1 h. After completion of reaction as indicated by TLC, reaction mixture was evaporated under vacuum. The crude was purified by prep-HPLC (Column: New X-bridge Prep, C18, OBD 19×250 mm, 5 μm Mobile phase: A=0.05% TFA in water, B=Acetonitrile, A:B=72:28, retention time 11.7 min) to give title compound as white solid (0.025 g, 16.93%). LCMS [M+H]⁺ for C₃₆H₄₇NO₉P found to be 668.4; ¹H NMR (400 MHZ, MeOD): δ: 7.47 (dd, J=10 & 2.0, 1H), 7.35 (d, J=7.6 Hz, 2H), 7.11 (d, J=8.0 Hz, 2H), 6.23 (d, 1H), 6.01 (s, 1H), 5.52 (s, 1H), 5.06 (d, J=4.8 Hz, 1H), 5.00-4.70 (m, 2H), 4 43 (brs, 1H), 3.60-3.50 (m, 1H), 1.80-1.60 (m, 20H), 1.51 (s, 3H), 1.20-1.10 (m, 1H), 1.03 (s, 3H), 1.00-0.95 (m, 1H).

### Synthesis 6-(4 ((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl)spiro[3.3]heptan-2-aminium 2,2,2-trifluoroacetate (INX-SM-21)

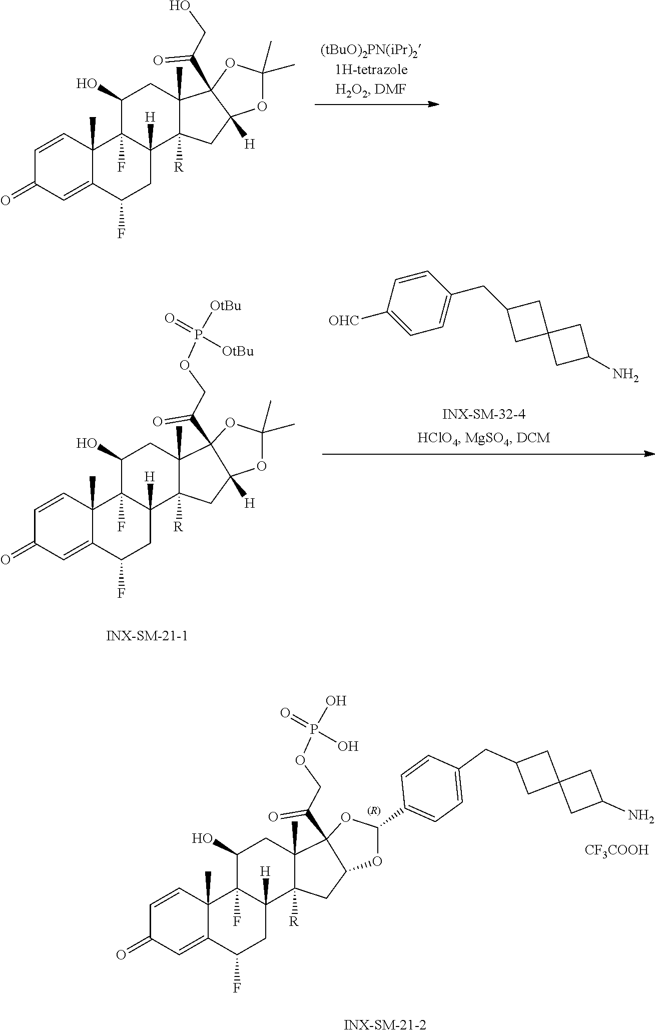

### Synthesis of di-tert-butyl (2-((2S,6aS,6bR,7S,8aS,8bS,11aR,12aS,12bS)-2,6b-difluoro-7-hydroxy-6a,8a,10, 10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) phosphate (INX-SM-21-1)

### Procedure

A 100 mL single-necked round bottom flask was charged with (2S,6aS,6bR,7S,8aS, 8bS,11aR,12aS,12bS)-2,6b-difluoro-7-hydroxy-8b-(2-hydroxyacetyl)-6a,8a,10, 10-tetramethyl-1, 2, 6a,6b,7,8,8a,8b,11a,12,12a,12b-dodecahydro-4H-naphtho[2′,1′:4,5]indeno[1,2-d][1,3]dioxol-4-one (1.0 g, 2.21 mmol), Di-tert-butyl N, N-Diisopropylphosphoramidite (14.7 g, 53.0 mmol) and DMF (1 mL). To this solution, 1H-tetrazole (1.5 g, 22.1 mmol) was added and allowed to stirred at room temperature for 24 h. The reaction mixture was cooled to 0° C. and hydrogen peroxide, (10 V) was added and stir further for 2 h at room temperature. After completion of reaction as indicated by TLC, reaction mixture was poured into saturated sodium thiosulphate solution and extracted with ethyl acetate. The combined organic layer was dried over Na₂SO₄ and evaporated under vacuum. The crude title compound was purified by reverse phase column chromatography (acetonitrile/water) to give the title (0.4 g, 28.08%). LCMS [M+H]⁺ for C₃₂H₄₈F₂O₉P found to be 645.3 [M+H]⁺.

### Synthesis of 6-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-aminium 2,2,2-trifluoroacetate (INX-SM-21)

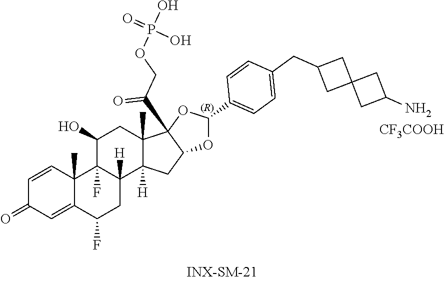

### Procedure

A 100 mL single-necked round bottom flask was charged with di-tert-butyl (2-((2S,6aS,6bR,7S,8aS,8bS,11aR,12aS,12bS)-2,6b-difluoro-7-hydroxy-6a,8a,10, 10-tetramethyl-4-oxo-1, 2, 4, 6a,6b,7,8,8a,11a,12,12a,12b-dodecahydro-8bH-naphtho[2′,1′:4,5]indeno[1,2-d][1,3]dioxol-8b-yl)-2-oxoethyl) phosphate (INX-SM-21-1) (0.2 g, 0.31 mmol) and tert-butyl (6-(4-formylbenzyl) spiro[3.3]heptan-2-yl)carbamate (0.1 g, 0.30 mmol) (INX-SM-32-4) in DCM (10 mL). MgSO₄ (0.18 g, 1.5 mmol) and HClO₄ (0.15 g, 1.5 mmol) were added at 0° C. and allowed to stir at room temperature for 4 hr. After completion of reaction as indicated by TLC, reaction mixture was poured into saturated solution of NaHCO₃ and extracted with 10% MeOH: DCM. The combined organic layer was dried over Na₂SO₄ and evaporated under vacuum. The crude was purified by prep-HPLC (Column: Shim-Pack Gist C18, 20×250 mm, 5 μm, Mobile phase: A=0.05% TFA in water, B=Acetonitrile, A:B=71:29, retention time 16.5 min) to give title compound as white solid (0.033 g, 15.6%). LCMS [M+H]⁺ for C₃₆H₄₅F₂NO₉P found to be 704.3; ¹H NMR (400 MHZ, MeOD): □: 7.40-7.35 (m, 3H), 7.15 (d, J=7.6 Hz, 2H), 6.37-6.34 (m, 2H), 5.67-5.52 (m, 1H), 5.54 (s, 1H, Acetal-H), 5.07 (m, J=3.6 Hz, 1H, C16-H), 4.95-4.72 (m, 2H), 4.35-4.30 (m, 1H), 3.70-3.58 (m, 1H), 2.78-1.63 (m, 19H), 1.61 (s, 3H), 1.03 (s, 3H).

### Synthesis of (S)-4-(2-(2-(((R)-2-amino-2-carboxyethyl) thio) acetamido)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX A23-CYS)

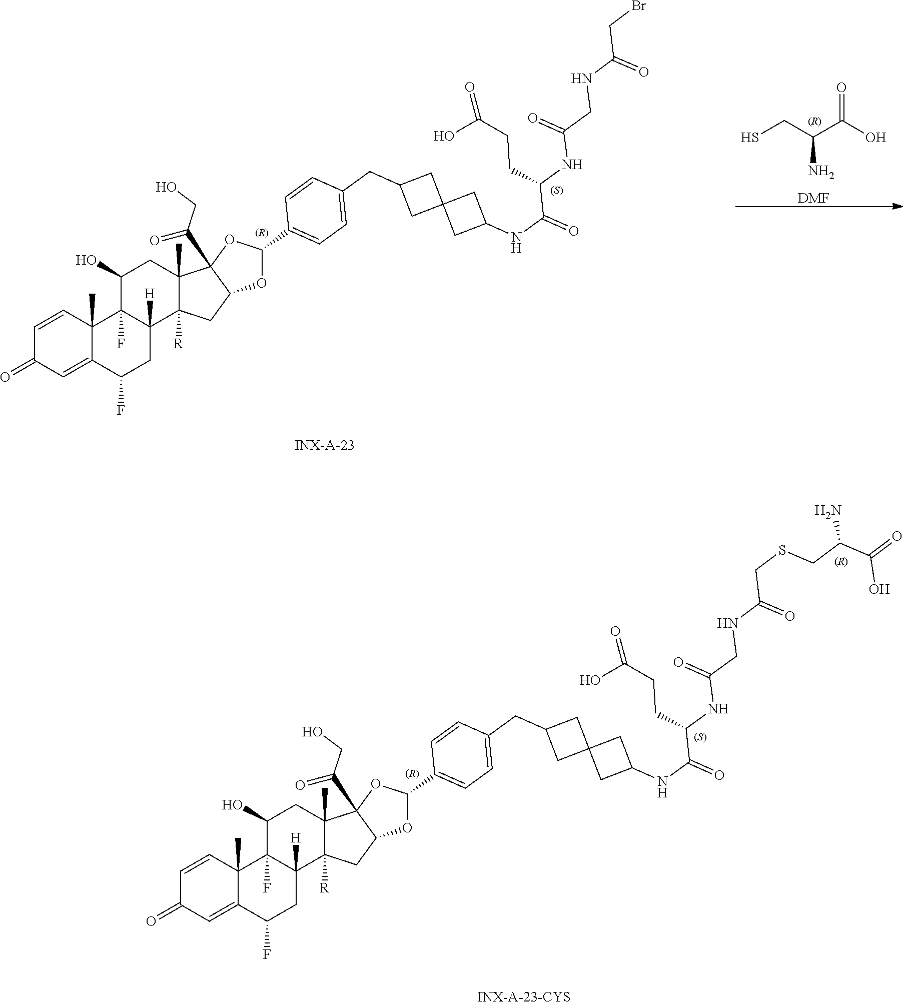

### Procedure

A 10 mL single-necked round bottom flash was charged with(S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX-A-23) (0.05 g, 0.053 mmol) and DMF (1 mL). To this solution, L-cysteine (0.009 g, 0.074 mmol) was added and stirred the reaction mixture for 2 h at room temperature. After completion of reaction as indicated by LCMS, reaction mixture was lyophilized and the crude was purified by prep-HPLC (Column: NEW Xbridge Prep, C18, OBD 19×250 mm, 5 μm Mobile phase: A=0.1% TFA in water, B=Acetonitrile, A:B=67:33, retention time 11.0 min) to give title compound as white solid (0.050 g, 15.98%). LCMS [M+H]⁺ for C₄₈H₆₁F₂N₄O₁₃S found to be 971.6; 1H NMR (400 MHZ, MeOD): δ: 7.36-7.34 (m, 3H), 7.16 (d, J=8.0, 2H), 6.37-6.32 (m, 2H), 5.70-5.48 (m, 1H), 5.48 (s, 1H, Acetal-H), 5.08 (d, J=4.0 Hz, 1H), 4.65 (d, 1H), 4.37-3.30 (m, 3H), 4.13-4.09 (m, 1H), 4.03-4.01 (m, 1H), 3.92 (s, 2H), 3.33 (s, 2H), 3.30-3.27 (m, 1H), 3.10-3.05 (m, 1H), 2.80-1.64 (m, 23H), 1.60 (s, 3H), 0.92 (s, 3H).

### Synthesis of (S)-4-(2-(2-(((R)-2-amino-2-carboxyethyl) thio) acetamido)acetamido)-5-((6-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX-A-12-CYS)

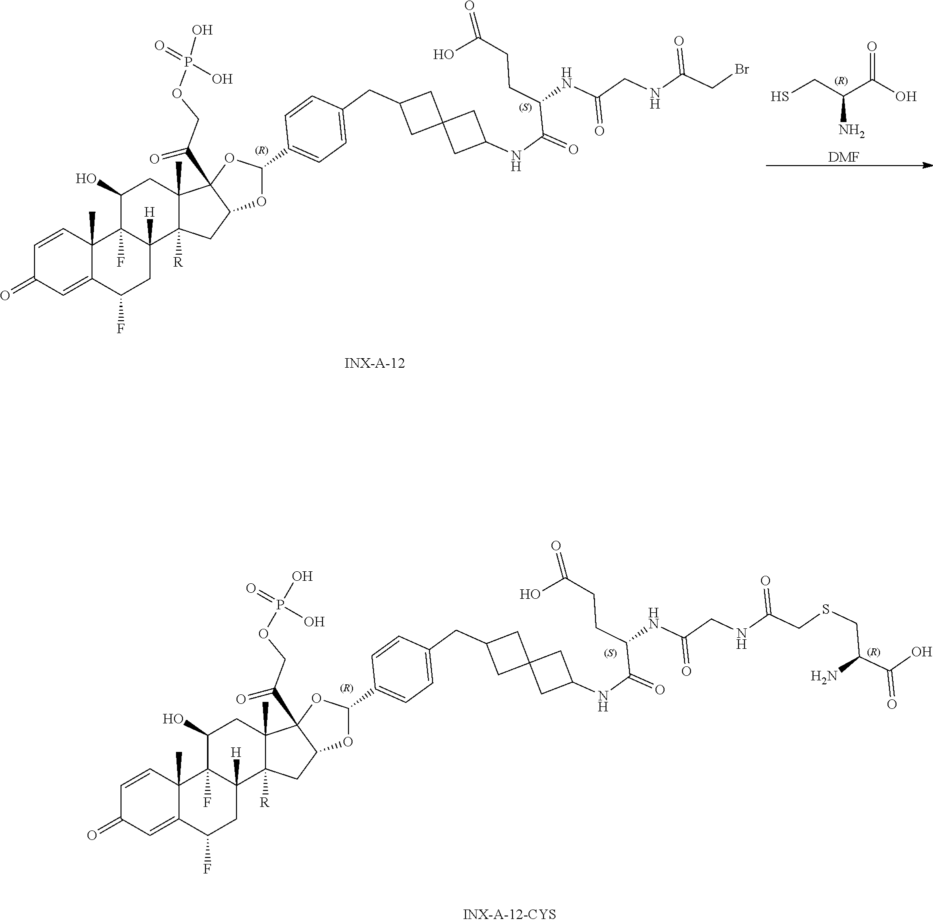

### Procedure

A 10 mL single-necked round bottom flash was charged with(S)-4-(2-(2-bromoacetamido)acetamido)-5-((6-(4-((2S,6aS,6bR,7S,8aS,8bS,10R,11aR,12aS,12bS)-2,6b-difluoro-7-hydroxy-6a,8a-dimethyl-4-oxo-8b-(2-(phosphonooxy)acetyl)-2,4,6a,6b,7,8,8a,8b,11a,12,12a,12b-dodecahydro-1H-naphtho[2′,1′:4,5]indeno[1,2-d][1,3]dioxol-10-yl)benzyl) spiro[3.3]heptan-2-yl)amino)-5-oxopentanoic acid (INX-A-12) (0.2 g, 0.19 mmol) and DMF (1 mL). To this solution, L-cysteine (0.035 g, 0.29 mmol) was added and stirred at room temperature for 16 h. After completion of reaction as indicated by LCMS, reaction mixture was lyophilized and the crude was purified by prep-HPLC (Column: new Xbridge Prep, C18, OBD 19×250 mm, 5 μm Mobile phase: A=0.05% TFA in water, B=Acetonitrile, A:B=70:30, retention time 11.2 min) to give title compound as white solid (0.025 g, 12.02%). LCMS [M+H]⁺ for C₄₈H₆₂F₂N₄O₁₆PS found to be 1052.4; ¹H NMR (400 MHz, MeOD) δ: 7.38-7.36 (m, 3H), 7.15 (d, J=8.0 Hz, 2H), 6.38-6.34 (m, 2H), 5.68-5.50 (m, 1H, CH—F), 5.53 (s, 1H, Acetal-H), 5.05 (d, J=4.0 Hz, 1H), 5.00-4.83 (m, 3H), 4.34-4.30 (m, 2H), 4.20-4.08 (m, 2H), 3.91 (s, 2H), 3.43 (s, 2H), 3.11-3.05 (m, 1H), 2.80-1.72 (m, 23H), 1.61 (s, 3H), 1.03 (s, 3H)

### Synthesis of S-(cyanomethyl) (2S,6aS,6bR,7S,8aS,8bS,10R,11aR,12aS,12bS)-10-(4-((6-aminospiro[3.3]heptan-2-yl)methyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxole-8b-carbothioate 2,2,2-trifluoroacetate (INX-SM-11)

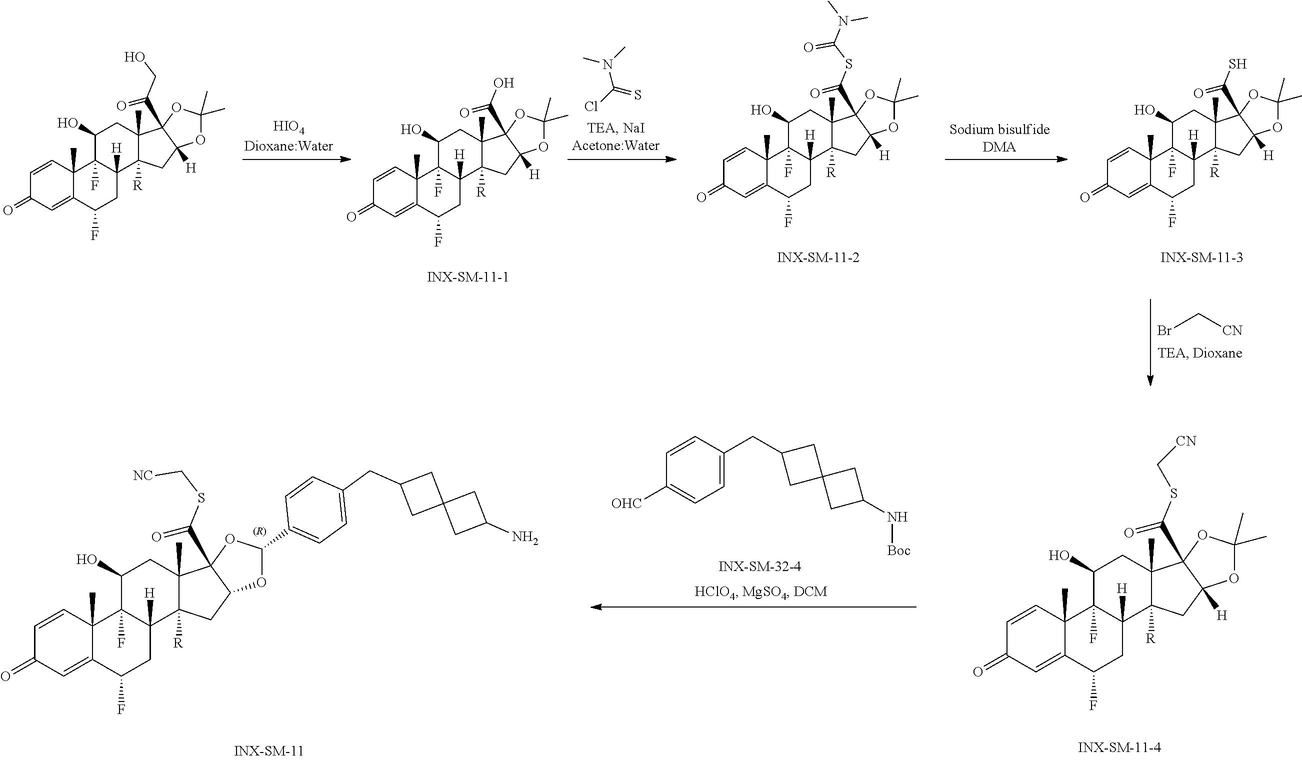

### Synthesis of (2S,6aS,6bR,7S,8aS,8bS,11aR,12aS,12bS)-2,6b-difluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxole-8b-carboxylic acid (INX-SM-11-1)

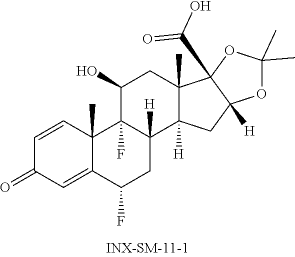

### Procedure

A 250 mL round bottom flask was charged with (2S,6aS,6bR,7S,8aS,8bS,11aR,12aS,12bS)-2,6b-difluoro-7-hydroxy-8b-(2-hydroxyacetyl)-6a,8a,10, 10-tetramethyl-1,2,6a,6b,7,8,8a,8b,11a,12,12a,12b-dodecahydro-4H-naphtho[2′,1′:4,5]indeno[1,2-d][1,3]dioxol-4-one (Fluocinolone acetonide) (5.0 g, 11.06 mmol) in 1,4-dioxane (80 mL). To this solution, HIO₄ (3.2 g, 33.18 mmol) in H₂O (20 mL) was added dropwise and stirred for 16 h at room temperature. After completion of reaction as indicated by TLC, reaction mixture was quenched with triethylamine and concentrated under reduced pressure. The crude was further poured into 2M aqueous NaOH solution and washed with ethyl acetate. The aqueous layer was acidified with 1N HCl and extracted with EtOAc. The combined organic layer was dried over Na₂SO₄ & concentrated under reduced pressure give title compound as yellow solid (4.0 g, 82.56%). LCMS [M+H]⁺ for C₂₃H₂₉F₂O₆ found to be 439.2.

### Synthesis of (2S,6aS,6bR,7S,8aS,8bS,11aR,12aS,12bS)-2,6b-difluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxole-8b-carboxylic dimethylcarbamic thioanhydride (INX-SM-11-2)

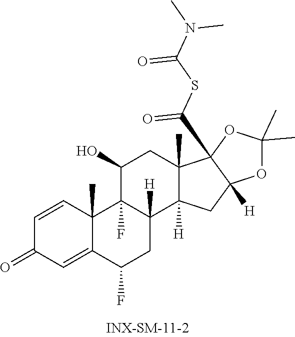

### Procedure

A 100 mL round bottom flask was charged with (2S,6aS,6bR,7S,8aS,8bS,11aR,12aS,12bS)-2,6b-difluoro-7-hydroxy-6a,8a,10, 10-tetramethyl-4-oxo-1,2,4,6a,6b,7,8,8a,11a,12,12a,12b-dodecahydro-8bH-naphtho[2′,1′:4,5]indeno[1,2-d][1,3]dioxole-8b-carboxylic acid (INX-SM-11-1) (4.0 g, 9.13 mmol) and acetone: H₂O (40:1 mL). To this solution, dimethylcarbamothioic chloride (2.24 g, 18.26 mmol), TEA (3.9 mL, 27.39 mmol) and NaI (0.272 g, 1.826 mmol) were added at room temperature and stirred for 16 h. After completion of reaction as indicated by TLC, reaction mixture was diluted with DMA to make it clear solution and then poured into chilled water. The resulted solid was filtered and dried under vacuum to give title compound as a yellow solid and taken to the next step without any further purification (2.0 g, 41.71%). LCMS [M+H]⁺ for C₂₆H₃₄F₂NO₆S found to be 526.2.

### Synthesis of (2S,6aS,6bR,7S,8aS,8bS,11aR,12aS,12bS)-2,6b-difluoro-7-hydroxy-6a,8a,10, 10-tetramethyl-4-oxo-1,2,4,6a,6b,7,8,8a,11a,12,12a,12b-dodecahydro-8bH-naphtho[2′,1′:4,5]indeno[1,2-d][1,3]dioxole-8b-carbothioic S-acid (INX-SM-11-3)

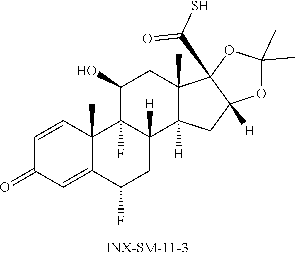

### Procedure

A 35 mL glass vial was charged with (2S,6aS,6bR,7S,8aS,8bS,11aR,12aS,12bS)-2,6b-difluoro-7-hydroxy-6a,8a,10, 10-tetramethyl-4-oxo-1,2,4,6a,6b,7,8,8a,11a,12,12a,12b-dodecahydro-8bH-naphtho[2′,1′:4,5]indeno[1,2-d][1,3]dioxole-8b-carboxylic dimethylcarbamic thioanhydride (INX-SM-11-2) (2.0 g, 3.809 mmol) and DMA (20 mL). To this solution, sodium bisulfide (2.13 g, 38.095 mmol) was added and stirred for 1 h at room temperature. After completion of reaction as indicated by TLC, reaction mixture was quenched with 1N HCl and extracted with ethyl acetate. The combined organic layer was dried over Na₂SO₄ and concentrated under reduced pressure to give title compound as yellow solid (0.500 g, 28.91%). LCMS [M+H]⁺ for C₂₃H₂₉F₂O₅S found to be 455.2.

### Synthesis of S-(cyanomethyl) (2S,6aS,6bR,7S,8aS,8bS,11aR,12aS,12bS)-2,6b-difluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxole-8b-carbothioate (INX-SM-11-4)

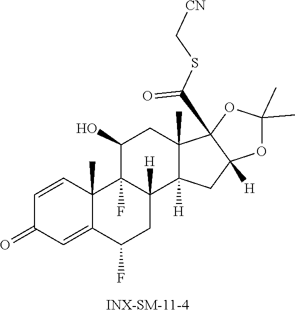

### Procedure

A 35 mL glass vial was charged with (2S,6aS,6bR,7S,8aS,8bS,11aR,12aS,12bS)-2,6b-difluoro-7-hydroxy-6a,8a,10, 10-tetramethyl-4-oxo-1,2,4,6a,6b,7,8,8a,11a,12,12a,12b-dodecahydro-8bH-naphtho[2′,1′:4,5]indeno[1,2-d][1,3]dioxole-8b-carbothioic S-acid (INX-SM-11-3) (0.500 g, 1.101 mmol) and 1,4-dioxane (5.0 mL). To this solution, TEA (0.3 mL, 2.20 mmol) and bromoacetonitrile (0.197 g, 1.651 mmol) were added and stirred for 16 h at room temperature. After completion of reaction as indicated by TLC, reaction mixture was poured into H₂O and extracted with ethyl acetate. The combined organic layer was dried over Na₂SO₄ and concentrated under reduced pressure. The crude was purified by column chromatography (ethyl acetate:hexane, 35:65) to give title compound as brown solid (0.17 g, 31.31%). LCMS [M+H]⁺ for C₂₅H₃₀F₂NO₅S found to be 494.2.

### Synthesis of S-(cyanomethyl) (2S,6aS,6bR,7S,8aS,8bS,10R,11aR,12aS,12bS)-10-(4-((6-aminospiro[3.3]heptan-2-yl)methyl)phenyl)-2,6b-difluoro-7-hydroxy-6a,8a-dimethyl-4-oxo-1,2,4,6a,6b,7,8,8a,11a,12,12a,12b-dodecahydro-8bH-naphtho[2′,1′:4,5]indeno[1,2-d][1,3]dioxole-8b-carbothioate 2,2,2-trifluoroacetate (INX-SM-11)

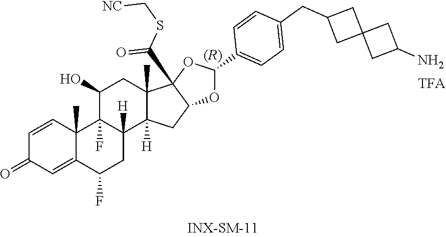

### Procedure

A 35 mL glass vial was charged with S-(cyanomethyl) (2S,6aS,6bR,7S,8aS,8bS,11aR,12aS,12bS)-2,6b-difluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxole-8b-carbothioate (INX-SM-11-4) (0.15 g, 0.30 mmol) and DCM (3 mL). To this solution, tert-butyl (6-(4-formylbenzyl) spiro[3.3]heptan-2-yl)carbamate (INX-SM-32-4) (0.100 g, 0.30 mmol), MgSO₄ (0.183 g, 1.52 mmol) and HClO₄ (0.152 g, 1.520 mmol) were added and stirred for 16 h at room temperature. After completion of reaction as indicated by TLC, reaction mixture was evaporated under vacuum and quenched with sat NaHCO₃. The solid was filtered and the crude was purified by prep-HPLC (Column: SHIM-PACK-GIST C18 (2508 (250×20) mm, 5 μm, Mobile phase: A=0.05% TFA in water, B=Acetonitrile, A:B=49:51, retention time 16.50 min) to give title compound as white solid (0.010 g, 4.37%). LCMS [M+H]⁺ for C₃₇H₄₃F₂N₂O₅S found to be 665.5; ¹H NMR (400 MHZ, MeOD): δ: 7.38-7.33 (m, 3H), 7.19 (d, J=8.0 Hz, 2H), 6.37-6.31 (m, 2H), 5.67-5.50 (m, 2H), 5.01 (d, 1H), 4.35-4.25 (m, 1H), 3.90 (dd, J=16.8 Hz, 2H), 3.61-3.59 (m, 1H), 2.70-1.67 (m, 19H), 1.59 (s, 3H), 1.12 (s, 3H).

### Example 4: Comparison of Binding and Internalization of Anti-VISTA Antibodies at Physiologic pH

Initially to assess whether VISTA antibodies would potentially effectively deliver steroids or other payloads into target immune cells studies were conducted to evaluate the internalization of different anti-VISTA antibodies into human monocytes. Specifically, the binding and internalization of naked anti-human VISTA antibodies (respectively INX200, 767 IgG1,3 (antibody sequences in FIG. 12) in human monocytes were compared. VISTA is highly expressed on most hematopoietic cells, particularly on myeloid cells. Based thereon we speculated that the rapid internalization combined with high density and selectivity for relevant cell types may make VISTA an ideal target for anti-inflammatory antibody drug conjugates (ADC).

Drug conjugates to anti-EGFR antibodies have been studied extensively as antibodies bound to EGFR are rapidly internalized into target cells. The literature details robust methods for determining internalization rates. For example, in EGFR-expressing cell lines, the LA22 mAb against EGFR reached the maximum internalization level of 65.8% within 10 min at 37° C. (Liu, Z., et al., (2009), “In-vitro internalization and in-vivo tumor uptake of anti-EGFR monoclonal antibody LA22 in A549 lung cancer cells and animal model”, Cancer Biother Radiopharm, 15-20), while Ab033 showed 54% internalization by 15 min (Durbin, K. R. et al., 2018, “Mechanistic Modeling of Antibody-Drug Conjugate Internalization at the Cellproton assignmentular Level Reveals Inefficient Processing Steps”, Mol Cancer Ther, 1535-7163).

The objective of the present studies was to evaluate the internalization rate of anti-VISTA monoclonal antibody INX200 in human monocytes and to further assess the internalization properties of 767-IgG1,3, in comparison to a pH-sensitive anti-human VISTA with enhanced serum PK half-life, developed by Five Prime Therapeutics and Bristol-Myers Squibb Company (Johnston, R. J. et al., (2019), “VISTA is an acidic pH-selective ligand for PSGL-1”, Nature, 574 (7779), 565-570).

### Materials and Methods

In this experiment the binding curves of anti-VISTA antibodies INX200, and 767-IgG1,3 to human monocytes (from freshly isolated human peripheral blood mononuclear cells or PBMCS) were first determined. Second, the internalization rates of these different antibodies on human monocytes were defined using as a negative control, the non-internalizing antibody anti-CD45. Briefly, to detect only internalized antibody, cells were first incubated with the fluorescently labelled antibody for 30 min at 4° C., a temperature at which little to no internalization can occur. Cells were washed and incubated at 25° C. to allow internalization. Cell surface signal was then quenched at various time points using equivalent amounts of anti-AF488 antibody. Subsequently, PBMCS were stained with anti-CD14 antibody to identify monocytes and analyzed by flow cytometry.

### Test Agents and Dosage

INX200 (Aragen, Lot #BP-2875-019-6.1) is a humanized anti-human VISTA antibody on a human IgG1/kappa backbone with L234A/L235A silencing mutations in the Fc region.
Human IgG1si (BioXcell ref, Lot #659518N1), is an anti-RSV (respiratory syncytial virus) antibody with a human IgG1/kappa backbone with L234A/L235A silencing mutations in the Fc region.
767-IgG1,3 (Aragen, Lot #BP-2985-019-6) is an anti-human VISTA antibody developed by Five Prime Therapeutics and Bristol-Myers Squibb Company on a human IgG1/kappa backbone with L234A/L235E/G237A silencing mutations in the Fc region. This antibody was designed to bind at low pH (e.g., pH 6) but to have minimal binding at physiological pH (pH 7.4), and as a result possesses an enhanced serum PK half-life due to not being subjected to TMDD as with other anti-VISTA antibodies. This antibody was made as described in the filing WO2018169993A1. The pH sensitive behavior of the antibody was confirmed via an ELISA format. Briefly 767-IgG1,3 or INX200 were plate bound and hIX50-biotin (VISTA ECD) diluted in citric acid/tween buffer with BSA was titered at pH 6.1, 6.7 or 7.5 and detected using a streptavidin-HRP conjugate/TMB readout. While the greatest INX200 binding was seen at pH 7.5, minimal binding of 767-IgG1,3 was observed at pH 7.5, with increasing levels of binding at pH 6.7, and even more at pH 6.1.
CD45, clone HI30 is an anti-human CD45 monoclonal antibody.
Anti-Alexa Fluor 488 (AF488) polyclonal antibody (Life Technologies, #A-11094) is an anti-Alexa Fluor 488 antibody used to quench AF488 fluorescent signal.

All antibodies, except anti-AF488, were conjugated with AF488 following the manufacturer's instructions for labeling and purification (Invitrogen Cat #A10235). Unless stated otherwise, antibodies were diluted in RPMI medium containing 1% BSA.

### PBMCs Preparation

Human PBMCs were isolated under sterile conditions from apheresis cones obtained from the Blood Donor Program at the Dartmouth Hitchcock Medical Center from healthy unrelated human donors. The blood was transferred to a 50 ml Falcon tube and diluted with PBS to 30 ml. 13 ml of Histopaque 1077 (Sigma Aldrich) was slowly layered under the blood, and tubes were centrifuged at 850×g for 20 min at RT with mild acceleration and no brake. Mononuclear cells were collected from the plasma/Ficoll interface, resuspended in 50 ml of PBS and centrifuged at 300×g for 5 min. Cells were resuspended in PBS and then counted.

### Fluorescent Labelling of the Antibodies

Anti-human VISTA antibodies and huIgG1si were conjugated with Alexa Fluor 488 dye following the manufacturer's instructions for labeling and purification (Invitrogen Cat #A10235). Concentration and degree of labeling were assessed via Nanodrop. The degree of labelling was 5.9 for INX200, and 7.1 for 767 IgG1,3. Anti-human CD45 (clone H130) conjugated to AF488 (Biolegend, #304017) and anti-CD14 to APC (clone M5E2, Biolegend, #301808) were used as is.

### Analysis of the Antibody Binding

PBMCs were resuspended at 5 x10⁶ cells/ml in RPMI/1% BSA buffer containing human Fc blocking reagent (eBioscience, 14-9161-73) and 50 μl/well of cells was then distributed to a 96-well plate. Anti-human VISTA antibodies were prepared in a 2× dilution series (10 concentrations) starting from 333 nM (50 μg/ml) in the RPMI/1% BSA buffer. PBMCS were stained for 30 min on ice to limit internalization, washed twice with PBS, and fixed with 2% FA in PBS for 10 min at 4° C. Monocytes were labelled with anti-CD14 mAb at 1:400 (v/v) in PBS/0.2% BSA for 20 min at RT. Cells were washed and analyzed by FACS, using a Macsquant (Miltenyi) flow cytometer and FlowJo for analysis. All graphs were prepared with GraphPad (Prism).

### Analysis of Antibody Internalization

5×10⁶ PBMCs were resuspended in 1 ml in RPMI/1% BSA buffer containing human Fc blocking reagent (eBioscience, 14-9161-73) and incubated with anti-human VISTA mAbs at 133 nM (20 μg/ml) for 30 min on ice. Cells were washed with 3 ml ice cold PBS and centrifuged for 2 min at 515×g. PBMCS were resuspended in 1.25 ml of fresh RPMI/1% BSA and kept at room temperature. Slowing down the internalization allowed to generate a robust curve. At each time point, 50 μl of cells were transferred to a 96 well plate containing 50 μl RPMI/1% BSA and anti-CD14 APC to measure total antibody bound. Cells were kept on ice to block subsequent internalization.

50 μl of cells were then transferred to a 96 well plate containing 50 μl RPMI/1% BSA, anti-AF488 antibody at 266 nM (40 μg/ml) to quench fluorescence of the surface bound antibody, and anti-CD14 APC to label monocytes. Cells were kept on ice to block subsequent internalization. Samples were collected in technical duplicates and the antibody internalization was followed for up to 60 min. At the end of the time course, all the samples were washed with PBS and fixed with 2% FA in PBS for 10 min at 4° C. After the last wash in PBS, cells were analyzed by FACS, using a Macsquant (Miltenyi) flow cytometer and FlowJo for analysis. The median fluorescence intensity (MFI) of the anti-VISTA or CD45 mAbs was measured and data plotted.

The intracellular fraction was calculated by subtracting the background fluorescence of untreated cells and normalizing the MFI values to the MFI at time=0. The internalization rate was calculated as a fraction of the intracellular signal to the total cell associated fluorescence at each timepoint (See equation below) and normalized to 100% (Liao-Chan, S. et al., (2015), “Quantitative assessment of antibody internalization with novel monoclonal antibodies against Alexa fluorophores”, PLOS One, 10 (4): e012470).

1−N₁−Q₁/N₁−N₁Q₀/N₀
N₁—Unquenched MFI at each time point (t1)
O₁—Quenched MFI at each timepoint (t1)
N₀—Unquenched MFI at 0 min (t0)
Q₀—Quenched MFI for the sample at 0 min (t0)

### Binding of the Naked Anti-VISTA Antibodies (INX200, 767-IgG1,3)

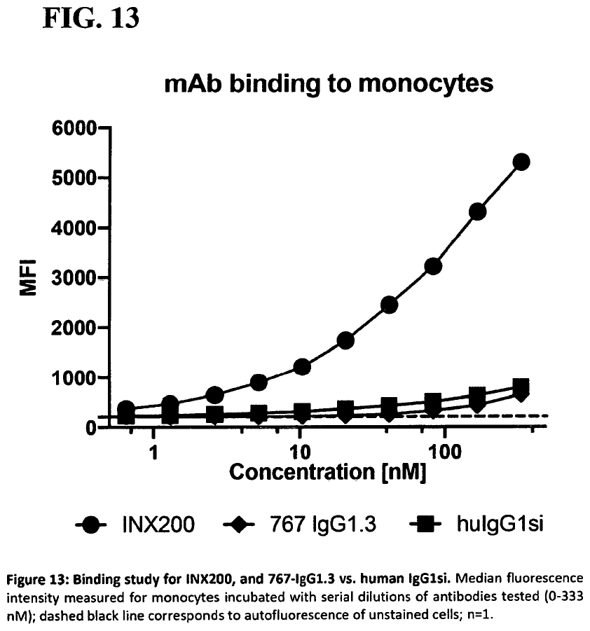

In the experiments in FIG. 13 the median fluorescence intensity was measured for monocytes incubated with serial dilutions of antibodies tested (0-333 nM); wherein the dashed black line corresponds to autofluorescence of unstained cells; n=1. A single measurement was taken at each concentration. As shown therein at the physiological pH of 7.3, INX200 showed concentration dependent increase in fluorescence within the range tested (0-333 nM) on CD14⁺ PBMCS (See FIG. 13). In contrast, no signal was detected when cells were incubated with 767-IgG1,3 antibody. This was expected due to its selectivity for binding at lower pH, which was confirmed previously in an ELISA format. The human IgG1si antibody used to assess the level of non-specific binding showed little to no binding even at high antibody concentration.

### Internalization of Anti-Human VISTA Antibodies

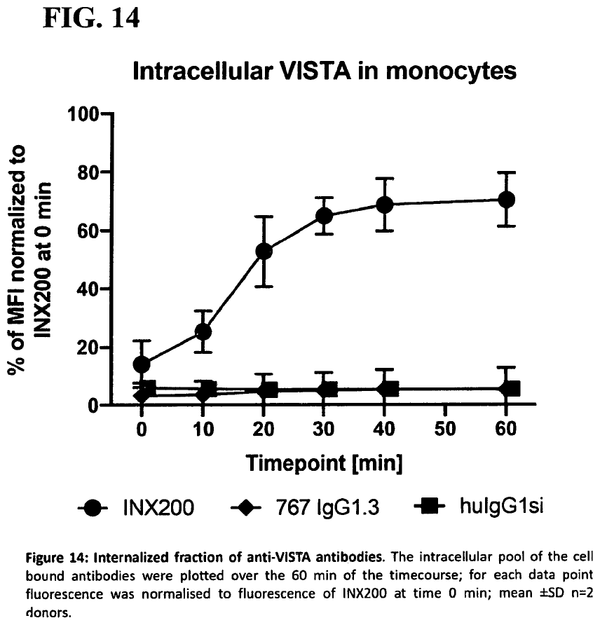

FIG. 14 shows the internalized fraction of anti-VISTA antibodies. In these experiments the intracellular pool of the cell bound antibodies were plotted over the 60 min of the timecourse; for each data point fluorescence was normalised to fluorescence of INX200 at time 0 min; mean±SD n=2 donors. In order to compare the internalized fraction of antibodies, the MFI values at each timepoint were corrected for background fluorescence by subtraction the MFI of untreated monocytes and normalized to the total MFI of the INX200 at t=0 timepoint (FIG. 14). Consistent with the data shown in FIG. 13, at t=0 767-IgG1,3 displayed weak binding with 3.2%+4.5% relative to INX200 (FIG. 14).

As shown therein nonspecific signal as represented by human IgG1si staining was assessed as 5.9% at 0 min. A clear increase in intracellular signal was observed over time when cells were incubated with INX200, and by 60 min the intracellular fraction was 70.4%+9.2%. The MFI values reached plateau by 40 min of the time course. By contrast, only 5.3%+7.5% of 767-IgG1,3 was detected as an internal fraction at 60 min. The intracellular signal of the human IgG1si was within 5-6% during the period of the time course.

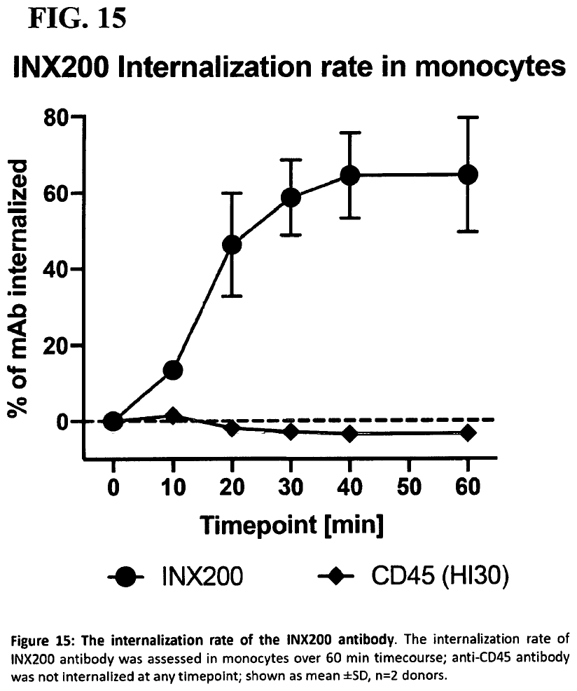

Additionally, in FIG. 15 another experiment was conducted wherein the internalization rate of the INX200 antibody was assessed in monocytes over 60 min time course and compared with the anti-CD45 antibody, HI30. As shown therein anti-CD45 antibody was not internalized at any timepoint; shown as mean±SD, n=2 donors. By contrast INX200 was efficiently internalized with half of the surface antibody detected intracellularly by 20 min (FIG. 15). Furthermore, within 40 min, 64.5%±11.2% of INX200 were internalized in monocytes. In contrast, no internalization of the anti-CD45 mAb was observed at any timepoint tested.

The data show that the anti-human VISTA INX200 binds with high affinity and is internalized with maximum internalization level of 64% by 40 minutes. This strongly suggests that VISTA is a uniquely suitable target for delivering anti-inflammatory payloads to immune cells, as these results suggest that a majority of payload should be delivered within a relatively short period of time, which is both nonobvious and nontrivial given the lack of CD45 internalization. By contrast, the pH sensitive antibody, anti-human VISTA 767.3-IgG1.3 has limited binding to monocytes at a physiological pH. Also, compared to INX200, 767-IgG1,3 displayed negligible to limited levels of internalization at a physiological pH.

### Example 5: Comparison of PK of Anti-VISTA Antibodies as Naked Antibody or Dexamethasone Conjugates Binding and Internalization of Exemplary Anti-VISTA Antibodies at Physiologic pH

Two experiments were conducted to compare the pharmacokinetics (PK) of the anti-human VISTA antibodies INX200 naked or conjugated to Dexamethasone (INX200A) in a first experiment (EXPERIMENT 1), and 767-IgG1.3 naked or conjugated to Dexamethasone (767-IgG1.3A) (Johnston et al, “VISTA is an acidic pH-selective ligand for PSGL-1.” Nature. 2019 October; 574 (7779): 565-5702019) in a second experiment (EXPERIMENT 2), in human VISTA knock-in (hVISTA KI) mice. These mice have the human VISTA cDNA knocked-in in place of the mouse VISTA gene, and express human VISTA both at RNA and protein levels. The experiments were performed in male hVISTA KI mice and in both studies the animals received 1 dose of antibody at 10 mg/Kg. Antibody amount in peripheral blood was quantified at 20 min, 4, 24, 48 hrs, and then at day 5, 8, 14, 21 and 28 for EXPERIMENT 1 and day 4, 7, 14, 21 and 28 for EXPERIMENT 2.

The objective of the these 2 experiments was to evaluate if the addition of 8 linker-payload molecules/antibody would modify the PK and confirm that the “pH sensitive” antibody described by BMS/Five Prime Therapeutics, and a glucocorticoid linked form have a significantly different PK (comparable to hlgG1) than anti-VISTA antibodies which bind to human VISTA expressing cells at physiologic and their respective glucocorticoid linked forms (short relative to hlgG1).

### Materials and Methods

### Experiment 1: PK Study for INX200, INX200A (Dexamethasone Conjugate) in Human VISTA KI Mice

The hVISTA KI mice were divided into 3 groups of 10 mice each, treated respectively with human IgG1, INX200 and INX200A at 10 mg/Kg on day 0.

### Experiment 2: PK Study for 767-IgG1.3, 767-IgG1.3A (Dexamethasone Conjugate) in Human VISTA KI Mice

The hVISTA KI mice were divided into 3 groups of 10 mice each, treated respectively with human IgG1, 767-IgG1.3 and 767-IgG1A at 10 mg/Kg on day 0. In both experiments, mice were bled retro-orbitally at 20 min, 4, 24, 48 hrs, and then at day 5 and 8 for EXPERIMENT 1 and day 4 and 7 for EXPERIMENT 2; circulating antibodies were quantified by ELISA.

### Test Agents and Dosage

INX200 (Aragen, Lot #BP-2875-019-6.1) is a humanized anti-human VISTA antibody on a human IgG1/kappa backbone with L234A/L235A silencing mutations in the Fc region.
INX200A (Abzena, Lot #JZ-0556-005) is the INX200 antibody with a drug/antibody ratio of 8, conjugated via the interchain disulfides. The linker/payload (A) consists of an esterase sensitive linker with a dexamethasone payload.
Human IgG1 (BioXcell ref, Lot #659518N1).
767-IgG1.3 (Aragen, Lot #BP-2985-019-6) is an anti-human VISTA antibody developed by Five Prime Therapeutics and Bristol-Myers Squibb Company on a human IgG1/kappa backbone with L234A/L235E/G237A silencing mutations in the Fc region. This antibody was designed to bind at low pH (e.g. pH 6) but to have minimal binding at physiological pH (pH 7.4) (1).
767-IgG1.3A (Abzena, Lot #JCC0624003) is the 767-IgG1.3 antibody with a drug/antibody ratio of 8, conjugated via the interchain disulfides. The linker/payload (A) consists of an esterase sensitive linker with a dexamethasone payload.

All antibodies were diluted in PBS and injected intravenously in the mouse tail vein in a volume of 0.2 ml to deliver a dose of 10 mg/Kg.

### Mice

The hVISTA mice were bred at Sage Labs (Boyertown, PA). The mice, aged 8-12 weeks, first transited for 3 weeks in our quarantine facility, and then were transferred to the regular facility. They were acclimated for 1 to 2 weeks prior to experiment initiation.

### Blood Draw and Preparation

Animals were bled no more than once every 24 hrs. Each mouse group was divided in 2 sub-groups of 5 mice that were bled alternatively on day 0. Blood was collected on day 0 post injection at 20 min, 4, 24, 48 hrs, and then at day 5 and 8 for EXPERIMENT 1 and day 4 and 7 for EXPERIMENT 2. In the first 24 hrs period, some data were excluded based on the registered quality of the intravenous injections. For subsequent time points, only animals that had successful intravenous injections were bled.

Peripheral blood was harvested from the retro-orbital cavity using a glass Pasteur pipette that was first rinsed with heparin to prevent coagulation. Blood was then centrifuged at 400 rcf for 5 min and plasma collected and stored at −80° C. for analysis (See above).

### Antibody Blood Concentration Analysis

### ELISA for Detection of Human IgG1

First, 96-well flat-bottom plates (Thermo Scientific Nunc Immunol Maxisorp, cat #442404) were coated with mouse anti-huIgG Fcγ (Jackson ImmunoResearch, cat #209-005-098) at 1 μg/ml in PBS for one hour at room temperature (RT).

The wells were washed 3 times with PT (PBS with 0.05% Tween 20) then blocked with PTB (PBS with 0.05% Tween 20 and 1% BSA) for 1 hour at RT. Human IgG (Southern Biotech, cat #0150-01) was used as a positive control and human IgG1 (BioXcell, cat #BE0297) was used to build a standard curve. The wells were washed 3 times with PT then plasma samples were incubated at up to 4 different dilutions in PTB (to fit on the standard curve) for 1 hour at RT.

After 3 washes with PT, mouse anti-human IgG Fcγ coupled to HRP (Jackson ImmunoResearch, cat #209-035-098), was used as detection reagent at a dilution of 1/2000 and incubated for 1 hour at RT. Following 3 washes, the ELISA reaction was revealed using TMB (Thermo Scientific, cat #34028) as a colorimetric substrate. After 5-10 min at RT, the reaction was stopped with 1M H₂SO₄.

### ELISA for Detection of INX200 or INX200A

First, 96-well flat-bottom plates (same as previous) were coated with hIX50 (human VISTA ECD, produced at Aragen Bioscience for ImmuNext) at 1 μg/ml in PBS for one hour at RT. After 3 washes, the wells were blocked with PTB for one hour at RT. INX908 (produced at Aragen Bioscience for ImmuNext) was used as a positive control and INX200 or INX200A was used to build a standard curve. The wells were washed 3 times with PT then plasma samples were incubated at up to 4 different dilutions in PTB (to fit on the standard curve) for 1 hour at RT.

After 3 washes with PT, mouse anti-human Kappa-HRP (Southern Biotech, cat #9230-05) was used at 1/2000 as a detection reagent, incubating 1 hour at RT. Following 3 washes, the ELISA reaction was revealed using TMB substrate. After 5 min at RT, the reaction was stopped with 1M H₂SO₄.

### ELISA for Detection of 767-IgG1.3 or 767-IgG1.3A

First, 96-well flat-bottom plates (same as above) were coated with mouse anti-huIgG Fcγ (Jackson ImmunoResearch, cat #209-005-098) at 1 μg/ml in PBS for one hour at RT.

After 3 washes, the wells were blocked with PTB for one hour at RT. Human IgG (Southern Biotech, cat #0150-01) was used as a positive control and 767-IgG1.3 or 767-IgG1.3A was used to build a standard curve. The wells were washed 3 times with PT then plasma samples were incubated at up to 4 different dilutions in PTB (to fit on the standard curve) for 1 hour at RT.

After 3 washes in PTB, mouse anti-human IgG Fcγ-HRP (Jackson ImmunoResearch, cat #209-035-098) was used at 1/2000 as a detection reagent, incubating 1 hour at RT. Following 3 washes, the ELISA reaction was revealed using TMB substrate following manufacturer instructions. After 5 min at RT, the reaction was stopped with 1M H₂SO₄. Antibody half-life was determined using the PKsolver program performing a non-compartmental analysis (NCA) after intravenous bolus.

### Results

### Experiment 1: INX200 Naked or Conjugated Antibody Plasma PK

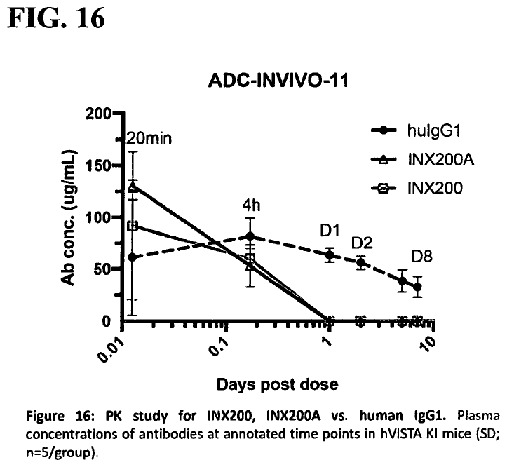

Plasma samples from the groups treated with INX200, INX200A or hlgG1 were collected to determine antibody concentration and subsequently their half-life. INX200 displayed a half-life of 0.1 day, which is lower but consistent with previous PK data (T_{1/2}=˜0.3 day), and the antibody was below quantification level at 24 hrs. INX200A displayed the same PK. In contrast, human IgG1 had a half-life of 7.2 days which is low but not atypical for an immunoglobulin (FIG. 16). The Figure shows plasma concentrations of antibodies at annotated time points in hVISTA KI mice (SD; n=5/group).

### Experiment 2:767-IgG1.3 Naked or Conjugated Antibody Plasma PK

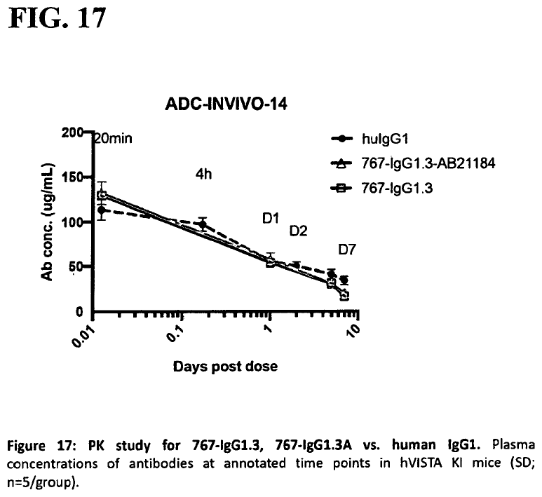

Plasma samples from the groups treated with 767-IgG1.3, 767-IgG1.3A or hlgG1 were collected to determine antibody concentration and subsequently their half-life. The results showed that 767-IgG1.3 and 767-IgG1.3A displayed similar half-life of respectively 3.5 and 4 days, and both were still detectable on day 7. The hlgG1 half-life was of 8.7 days, similar to what was observed in EXPERIMENT 1 (See FIG. 17 which contains the PK study for 767-IgG1.3, 767-IgG1.3A vs. human IgG1 and wherein plasma concentrations of antibodies at annotated time points in hVISTA KI mice (SD; n=5/group)).

### Conclusions

The results of these 2 experiments show that:

### Experiment 1

The data in FIG. 16 show that the anti-human VISTA antibody INX200 (which binds to human VISTA cells at physiological pH) is not quantifiable in plasma at 24 hrs post dosing due to target mediated drug disposition (TMDD) while the human IgG1 control shows the more typical extended half-life for an IgG. The results further show that conjugation of dexamethasone at DAR=8 to INX200 does not affect its PK.

### Experiment 2

The data in FIG. 17 show that the pH sensitive anti-human VISTA 767-IgG1.3 exhibits a PK similar to the human IgG1 control antibody arguing that it is has limited binding of its VISTA target and is not subjected to TMDD. Also, conjugation of dexamethasone at DAR=8 to 767-IgG1.3 does not affect its PK.

### Example 6: Long Term Impact of Antibody Drug Conjugates on Ex Vivo Macrophage Activation

In this example experiments were conducted to assess the long term efficacy of an exemplary inventive antibody drug conjugate (ADC) molecule which comprises an antibody that targets VISTA, a cell surface molecule highly expressed on most hematopoietic cells, including myeloid and T cells, and a glucocorticoid (GC) drug. We have previously shown (internal non-published studies) that such ADCs exert robust anti-inflammatory activity in short term inflammation models. The purpose of these studies was (i) to evaluate the pharmacodynamic range of various antibody drug conjugates (ADCs) and anti-human VISTA monoclonal antibodies linked to a glucocorticoid (GC) payload in myeloid cells; and (ii) evaluate the potency of exemplary INX GC linker payload ADCs.

First, we evaluated long-term in vivo impact of ADC on an early GC response gene, FKBP5 (Vermeer et al. (2003) “Glucocorticoid-induced increase in lymphocytic FKBP51 messenger ribonucleic acid expression: a potential marker for glucocorticoid sensitivity, potency, and bioavailability”, J Clin Endocrinol Metab. 88 (1): 277-84), as compared to Dexamethasone (Dex) on peritoneal resident macrophages (PRM) and spleen monocytes.

Based thereon we developed a model to allow us to evaluate long-term anti-inflammatory impact of ADC on specific target populations, such as PRMs. Briefly, ADCs were delivered in vivo via intraperitoneal (i.p.) injection, and after 1 to 7 days PRMs were isolated and put in culture. In the absence of GC treatment, after 2h PRMs become highly activated as shown by increases in cytokine production. Dex treatment in vivo 2h before PRM isolation robustly reduces cytokine production. The objective of these studies was to evaluate the efficacy and pharmacodynamic range of INX human VISTA antibodies conjugated to a glucocorticoid payload as compared to free Dex.

### Materials and Methods

Method for Assessing ADC or Dex impact on FKBP5 transcription in PRMs and spleen monocytes

Dex was injected i.p. 2 to 24h before mouse euthanasia and cell isolation. ADCs were then injected from 17h to 7 days before mouse euthanasia and cell isolation.

### Test Agents and Dosage

### Antibodies

INX201 (Aragen, Lot #BP-3200-019-6), is a humanized anti-human VISTA antibody on a human IgG1/kappa backbone with L234A/L235A/E269R/K322A silencing mutations in the Fc region.
INX201J (Abzena, Lot #s JZ-0556-025-1, JZ-0556-027, JZ-0556-013) is the INX201 antibody with a drug/antibody ratio (DAR) of 8.0, conjugated via full modification of the interchain disulfides. The linker/payload (J) is based on a previously reported linker/payload (U.S. Ser. No. 15/611,037). It consists of a negatively charged protease sensitive linker with a budesonide analog payload (INX J-2).
INX231J (Abzena, Lot #JZ-0556-013-1) is INX231 conjugated with a DAR of 8.0. The linker/payload (INX J) is a negatively charged protease sensitive linker with a budesonide analog payload (INX J-2).
INX234J (Abzena, Lot #JZ-0556-013-2) is INX234 conjugated with a DAR of 8.0. The linker/payload (INX J) is a negatively charged protease sensitive linker with a budesonide analog payload (INX J-2).
INX240J (Abzena, Lot #JZ-0556-013-3) is INX240 conjugated with a DAR of 8.0. The linker/payload (INX J) consists of a negatively charged protease sensitive linker with a budesonide analog payload (INX J-2).
INX201O (Abzena, Lot #JZ-0556-016-2) is INX201 with a DAR of 8.0, conjugated via full modification of the interchain disulfides. The linker/payload (INX O) consists of a negatively charged protease sensitive linker with a budesonide analog payload (INX-SM-4).
INX201P (Abzena, Lot #JZ-0556-016-1) is INX201 with a DAR of 8.0, conjugated via full modification of the interchain disulfides. The linker/payload (INX P) consists of a negatively charged protease sensitive linker with a budesonide analog payload (INX-SM-3).
INX233 (ATUM Lot #82276.1.a) is a humanized anti-human VISTA antibody on a human IgG1/kappa backbone with L234A/L235A/E269R/K322A silencing mutations in the Fc region.
INX233P (Abzena, Lot #PP-0924-001-3) is INX233 with a DAR of 8.0, conjugated via full modification of the interchain disulfides. The linker/payload (INX P) consists of a negatively charged protease sensitive linker with a budesonide analog payload (INX-SM-3).
INX231 (ATUM Lot #72928.1.a) is a humanized anti-human VISTA antibody on a human IgG1/kappa backbone with L234A/L235A/E269R/K322A silencing mutations in the Fc region.
INX231P (Abzena, lot #JZ-0556-017-1) is INX231 with a DAR of 8.0, conjugated via full modification of the interchain disulfides. The linker/payload (INX P) consists of a negatively charged protease sensitive linker with a budesonide analog payload (INX-SM-3).
INX234 (ATUM Lot #72931.2.a) is a humanized anti-human VISTA antibody on a human IgG1/kappa backbone with L234A/L235A/E269R/K322A silencing mutations in the Fc region.
INX234P (Abzena, lot #JZ-0556-017-2) is INX234 with a DAR of 8.0, conjugated via full modification of the interchain disulfides. The linker/payload (INX P) consists of a negatively charged protease sensitive linker with a budesonide analog payload (INX-SM-3).
INX240 (ATUM Lot #73419.2.a) is a humanized anti-human VISTA antibody on a human IgG1/kappa backbone with L234A/L235A/E269R/K322A silencing mutations in the Fc region.
INX240P (Abzena, lot #JZ-0556-017-3) is INX240 with a DAR of 8.0, conjugated via full modification of the interchain disulfides. The linker/payload (INX P) consists of a negatively charged protease sensitive linker with a budesonide analog payload (INX-SM-3).
INX231R (Abzena, Lot #PP-0924-001-2) is INX231 with a DAR of 8.0, conjugated via full modification of the interchain disulfides. The linker/payload (INX R) consists of a neutral protease sensitive linker with a budesonide analog payload (INX-SM-3).
INX231S (Abzena, lot #PP-0920-014-1) is INX231 with a DAR of 6.9, conjugated via modification of the interchain disulfides. The linker/payload (INX S) consists of a negatively charged protease sensitive linker with a fluocinolone acetonide analog payload (INX-SM-24).
INX231V (Abzena, lot #PP-0920-014-2) is INX231 with a DAR of 7.8, conjugated via modification of the interchain disulfides. The linker/payload (INX V) consists of a negatively charged protease sensitive linker with a budesonide analog payload (INX-SM-32).
INX231W (Abzena, lot #PP-0920-014-3) is INX231 with a DAR of 7.5, conjugated via modification of the interchain disulfides. The linker/payload (INX W) consists of a positively charged protease sensitive linker with a budesonide analog payload (INX-SM-3).
INX234A3 (Abzena, Lot #PP-0924-023-1) is the INX234 antibody with a drug/antibody ratio (DAR) of 8.0, conjugated via full modification of the interchain disulfides. The linker/payload (INX A3) consists of a positively charged protease sensitive linker with a budesonide analog payload (INX-SM-32).
INX234A4 (Abzena, Lot #PP-0924-023-2) is the INX234 antibody with a drug/antibody ratio (DAR) of 7.9, conjugated via full modification of the interchain disulfides. The linker/payload (INX A4) consists of a negatively charged protease sensitive linker with a budesonide analog payload (INX-SM-43).
INX234T (Abzena, Lot #PP-0924-023-3) is the INX234 antibody with a drug/antibody ratio (DAR) of 8.0, conjugated via full modification of the interchain disulfides. The linker/payload (INX T) consists of a negatively charged protease sensitive linker with a budesonide analog payload (INX-SM-3) that is phosphorylated.
INX201L (Abzena, Lot #JZ-0556-026-1) is the INX201 antibody with a drug/antibody ratio (DAR) of 8.0, conjugated via full modification of the interchain disulfides. The linker/payload (J) consists of a negatively charged protease sensitive linker with a budesonide analog payload (INX J-2) that is phosphorylated.
INX234V (Abzena, Lot #RJS-1054-003) is the INX234 antibody with a drug/antibody ratio (DAR) of 8.0, conjugated via full modification of the interchain disulfides. The linker/payload (INX V) consists of a negatively charged protease sensitive linker with a budesonide analog payload (INX-SM-32).
INX234A5 (Abzena, Lot #RJS-1054-002) is the INX234 antibody with a drug/antibody ratio (DAR) of 7.9, conjugated via full modification of the interchain disulfides. The linker/payload (INX A5) consists of a negatively charged protease sensitive linker with a budesonide analog payload (INX-SM-44).
INX234A11 (Abzena, Lot #RJS-1054-001) is the INX234 antibody with a drug/antibody ratio (DAR) of 8.0, conjugated via full modification of the interchain disulfides. The linker/payload (INX A11) consists of a negatively charged protease sensitive linker (Asn/gly) with a budesonide analog payload (INX-SM-32).
INX231A7 (Abzena, Lot #RJS-1054-007-001) is the INX234 antibody with a drug/antibody ratio (DAR) of 7.8, conjugated via full modification of the interchain disulfides. The linker/payload (INX A7) consists of a negatively charged protease sensitive linker with a budesonide analog payload (INX-SM-32) that is phosphorylated.
INX231A12 (Abzena, Lot #RJS-1054-007-002) is the INX234 antibody with a drug/antibody ratio (DAR) of 6.99, conjugated via full modification of the interchain disulfides. The linker/payload (INX A12) consists of a negatively charged protease sensitive linker with a fluocinolone acetonide analog payload (INX-SM-25) that is phosphorylated.
INX231A23 (Abzena, Lot #RJS-1054-006-001) is the INX234 antibody with a drug/antibody ratio (DAR) of 7.34, conjugated via full modification of the interchain disulfides. The linker/payload (INX A23) consists of a negatively charged protease sensitive linker with a fluocinolone acetonide analog payload (INX-SM-25).
The antibodies were diluted in PBS and injected intraperitoneal (i.p.) in a volume of 0.2 ml to deliver a specified dose.

### Dexamethasone

Dexamethasone sterile injection from Phoenix, NDC 57319-519-05, was diluted in PBS and dosed as described via i.p. injection.

### Mice

The hVISTA mice were bred on site (Center for Comparative Medicine and Research at Dartmouth). All the experiments were done in female mice enrolled between 9 and 15 weeks of age.

### Cell Isolation

After euthanasia, mice were injected in the peritoneal cavity with 7 ml of PBS/0.5% BSA/2 mM EDTA. After a brief massage of the peritoneum, a small incision was performed and the peritoneal lavage collected. PRM were isolated using negative selection (Miltenyi kit, ref 130-110-434). Spleen were dissected and dissociated mechanically; monocytes were isolated using negative selection (Stem Cell, EasySep™ Mouse CD11b Positive Selection Kit II).

### RNA Preparation and Real Time PCR

Cell pellets from different tissues were resuspended in 0.4 ml RNeasy lysis buffer from RNeasy Plus Mini kit (Qiagen, PN: 74136) and homogenize with 20G needle for 5 times. RNA was isolated following manufacturer's instructions and RNA eluted in in 30 or 40 ml H₂O (RNase/DNase free). RNA concentration was assessed on Nanodrop.

Reverse transcription was done using Taqman reverse transcription reagents (#N8080234) and following manufacturer's instructions. Quantitative Real-Time PCR was done using Taqman master mix 2× kit (#4369016) and Taqman primers for mouse FKBP5 (Mm00487401_m1), and mouse HPRT as housekeeping gene (Mm446968_m1) and run on a QuantStudio3 from Applied Biosystem.

Ct data were converted to DCt (FKBP5 normalized to HPRT within a sample) and then ΔΔCt (FKBP5 relative levels for treated sample vs PBS control) to obtain Log 2 fold-changes relative to PBS.

### Peritoneal Resident Macrophage Culture and Cytokine Analyses

### Culture Conditions

PRMs were resuspended in RPMI 1640 with 10% FBS, 10 mM Hepes, Penicillin/Streptomycin and glutamine and 100,000 cells were plated per well in a 96-well tissue culture plate. Supernatants were collected at 2 and 24h post plating and stored at −80° C.

### Cytokine Analyses Using a Millipore Platform

Cytokine analyses were conducted on 25 ml of plasma using a Millipore mouse 32-plex platform. The Immune Monitoring Lab (IML, Shared Resources at Dartmouth-Hitchcock Norris Cotton Cancer Center) performed the analyses.

### Cytokine Analyses Via ELISA

BioLegend (cat #430904) ELISA MAX Deluxe Set Mouse TNF-α
BioLegend (cat #431304) ELISA MAX Deluxe Set Mouse IL-6

ELISAs were conducted following the manufacturer's included protocol.

### Results

### Experiment 1: FKBP5 Transcriptional Activation Following Dex Treatment in Peritoneal Resident Macrophages and Spleen Monocytes

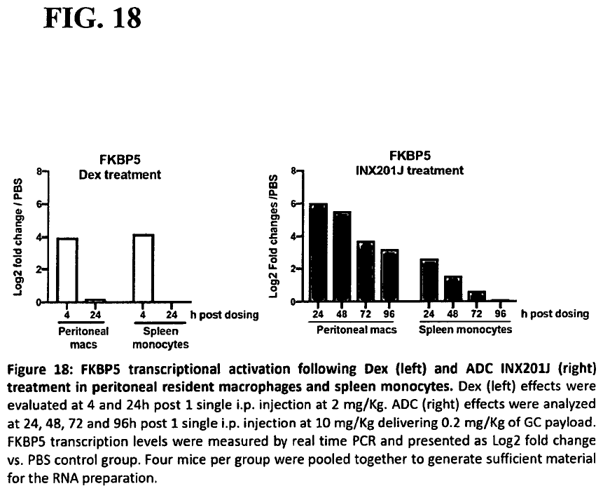

The experiment in FIG. 18 compares the effects FKBP5 transcriptional activation following Dex (left) and ADC INX201J (right)) treatment in peritoneal resident macrophages and spleen monocytes. As shown therein Dex (left) effects were evaluated at 4 and 24h post 1 single i.p. injection at 2 mg/Kg; ADC (right) effects were analyzed at 24, 48, 72 and 96h post 1 single i.p. injection at 10 mg/Kg delivering 0.2 mg/Kg of GC payload. FKBP5 transcription levels were measured by real time PCR and presented as Log 2 fold change vs. PBS control group. Four mice per group were pooled together to generate sufficient material for the RNA preparation. The data in FIG. 18 show that Dex treatment causes a dramatic increase in FKBP5 messenger RNA, in both PRM and spleen monocytes, by 4h post treatment but that the transcriptional impact is gone by 24h (FIG. 18, left). In contrast, INX201J's impact on FKBP5 is long-lasting, i.e., increases in FKBP5 messenger RNA are detected as late as 96h in PRM and 72h in spleen monocytes (FIG. 18, right).

In the absence of any added stimulus, when PRMs are transferred on tissue culture plates, they become very rapidly activated and massively increase the production of numerous pro-inflammatory cytokines that can be measured from cell supernatants as early as 1h post plating.

### Experiment 2: Dex Treatment Prevents the Ex Vivo Induction of Pro-Inflammatory Cytokines in PRMs

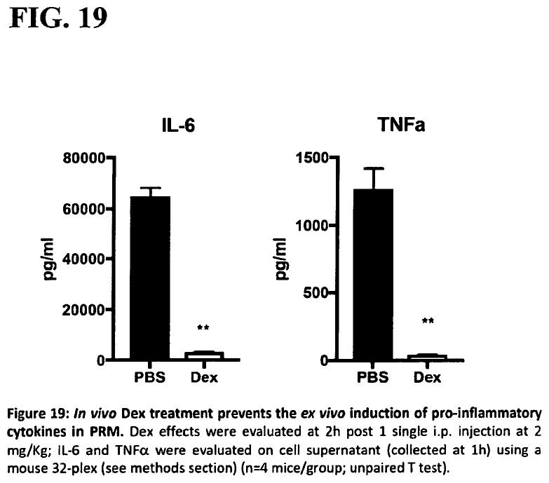

The experiment in FIG. 19 shows that Dex treatment prevents the ex vivo induction of pro-inflammatory cytokines in PRM. In the experiment Dex effects were evaluated at 2h post 1 single i.p. injection at 2 mg/Kg; IL-6 and TNFα were evaluated on cell supernatant (collected at 1h) using a mouse 32-plex (See methods section) (n=4 mice/group; unpaired T test)

These results show that Dex treatment in vivo, 2h before cell isolation, can robustly shut down the ex vivo PRM activation exemplified here by IL-6 and TNFα secretion, as early as 1h post plating of the cells. This impact is still clearly detected after 24h in culture (not shown).

### Experiment 3: Pharmacodynamics of ADC INX201J

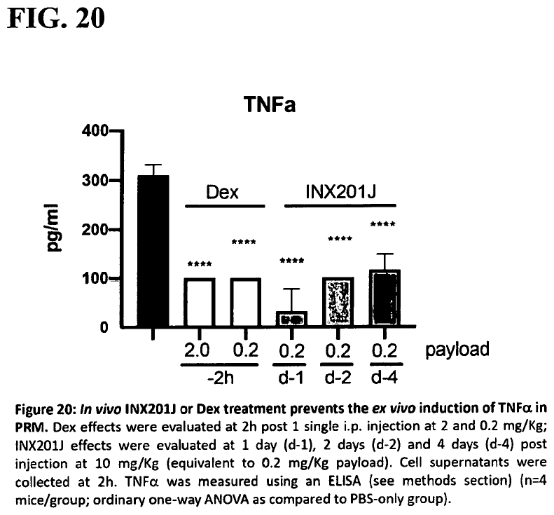

In the experiment contained in FIG. 20, the pharmacodynamic range of the ADC INX201J was evaluated with the ADC being injected at day −4,−2 and −1 before isolation of PRM and ex vivo stimulation. The Dex control groups were injected 2h before PRM isolation. Therein Dex effects were evaluated at 2h post 1 single i.p. injection at 2 and 0.2 mg/Kg; INX201J effects were evaluated at 1 day (d-1), 2 days (d-2) and 4 days (d-4) post injection at 10 mg/Kg (equivalent to 0.2 mg/Kg payload). Cell supernatants were collected at 2h. TNFα was measured using an ELISA (See methods section) (n=4 mice/group; ordinary one-way ANOVA as compared to PBS-only group). INX201J was dosed at 10 mg/Kg or equivalent 0.2 mg/Kg of payload.

As shown in FIG. 20, INX201J treatment had a highly significant impact on reducing TNFα production when dosed on day −1. Additionally, dosing of the ADC instead on day −2 or −4 still impacted secreted cytokine levels similarly to the Dex control groups. The data suggest that INX201J elicits a long term (>4 days) anti-inflammatory impact on PRM. Notably the amount of detected TNFα in the supernatant in this experiment is much lower than in the previous experiment, probably due to the fact that the quantification was done by ELISA (instead of Luminex). We also observed lower IL-6 levels when quantification was done by ELISA in other experiments.

### Experiment 3: Long-Term Potency of Different Anti-VISTA Antibodies Conjugated to the J Payload

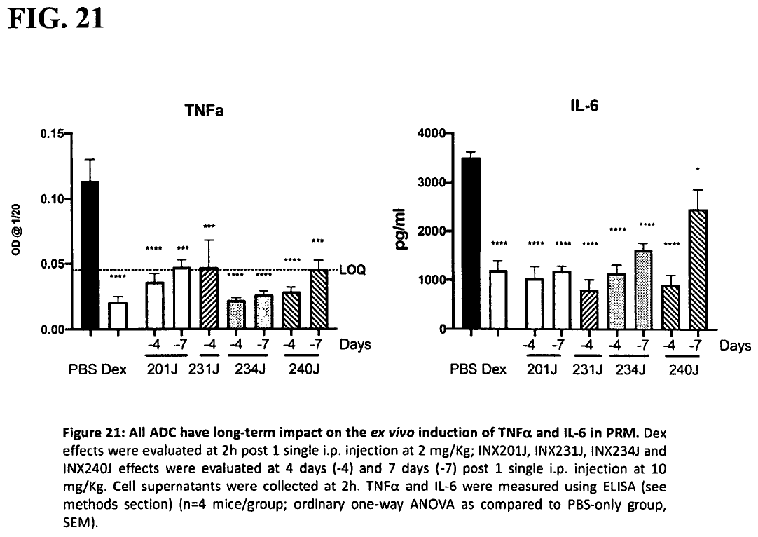

In the experiment in FIG. 21 the inventors evaluated long-term potency of different anti-VISTA antibodies conjugated to the J payload. INX201J, INX234J, and INX240J were injected on day −4 and day −7 at 10 mg/Kg (0.2 mg/Kg of payload) while Dex was dosed at 2 mg/Kg, 2 h before cell isolation. For practical experimental reasons, INX231J was only dosed on day −4.

The results in FIG. 21 revealed that the tested ADCs have long-term impact on the ex vivo induction of TNFα and IL-6 in PRM. Therein Dex effects were evaluated at 2h post 1 single i.p. injection at 2 mg/Kg; INX201J, INX231J, INX234J and INX240J effects were evaluated at 4 days (−4) and 7 days (−7) post 1 single i.p. injection at 10 mg/Kg. Cell supernatants were collected at 2h. TNFα and IL-6 were measured using ELISA (See methods section) (n=4 mice/group; ordinary one-way ANOVA as compared to PBS-only group). As shown in FIG. 21, for both TNFα and IL-6, all 4 ADCs displayed potent anti-inflammatory activity when dosed at either day −4 or −7.

### Experiment 5: Dose-Dependent Impact of INX231J, INX234J and INX240 J on Ex Vivo PRM Activation

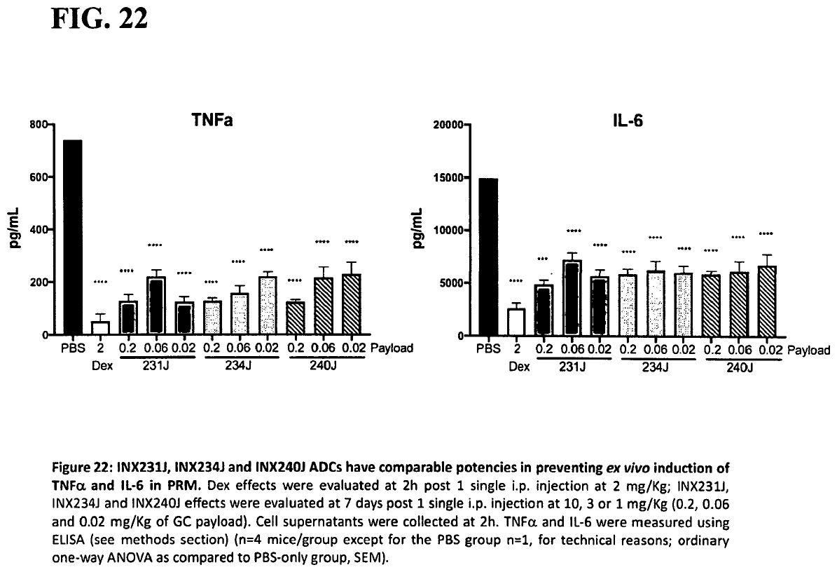

In the experiment in FIG. 22 the dose-dependent impact of INX231J, INX234J and INX240J on ex vivo PRM activation was evaluated. In these experiments Dex effects were evaluated at 2h post 1 single i.p. injection at 2 mg/Kg; INX231J, INX234J and INX240J effects were evaluated at 7 days post 1 single i.p. injection at 10, 3 or 1 mg/Kg (0.2, 0.06 and 0.02 mg/Kg of GC payload). Cell supernatants were collected at 2h. TNFα and IL-6 were measured using ELISA (See methods section) (n=4 mice/group except for the PBS group n=1, for technical reasons; ordinary one-way ANOVA as compared to PBS-only group).

As noted all ADCs were injected i.p. on day −7 at different doses: 10, 3 and 1 mg/Kg delivering respectively 0.2, 0.06 and 0.02 mg/Kg of GC payload. Dex was dosed at 2 mg/Kg 2h before cell isolation. The results showed that no significant differences were observed between the different ADCs, suggesting they have similar potencies (FIG. 22).

### Experiment 6: Potency of Exemplary Inventive ADCs as Compared to Dex on Day 7 Post Treatment and INX201 Conjugated to Linker/Payload P

In the experiment in FIG. 23, the potency of 1) the J-linked ADCs as compared to Dex on day 7 post treatment, and 2) INX201 conjugated to linker/payload P was evaluated. The results in FIG. 23 indicate that INX201J, INX201P, INX231J, INX234J and INX240J ADCs have comparable potencies in preventing ex vivo induction of TNFα and IL-6 in PRM. In the experiments INX201J, INX201P, INX231J, INX234J, INX240J and Dex effects were evaluated at 7 days post 1 single i.p. injection; ADCs were dosed at 10 mg/Kg (0.2 mg/Kg of GC payload) and Dex at 2 mg/Kg. Cell supernatants were collected at 2h. TNFα and IL-6 were measured using as above-described (n=4 mice/group except for the PBS and Dex groups with n=3, for technical reasons; ordinary one-way ANOVA as compared to PBS-only group).

As noted, all treatments were injected i.p. on day −7, with Dex at 2 mg/Kg and the ADCs at 0.2 mg/Kg of payload. The data in FIG. 23 showed that while Dex has lost all efficacy in controlling the cytokine response, all the ADCs carrying the J or P payload have comparable potency.

### Experiment 7: Potency of Exemplary Inventive ADCs as Compared to Dex on Day 7 Post Treatment and INX201 Conjugated to Linker/Payload P

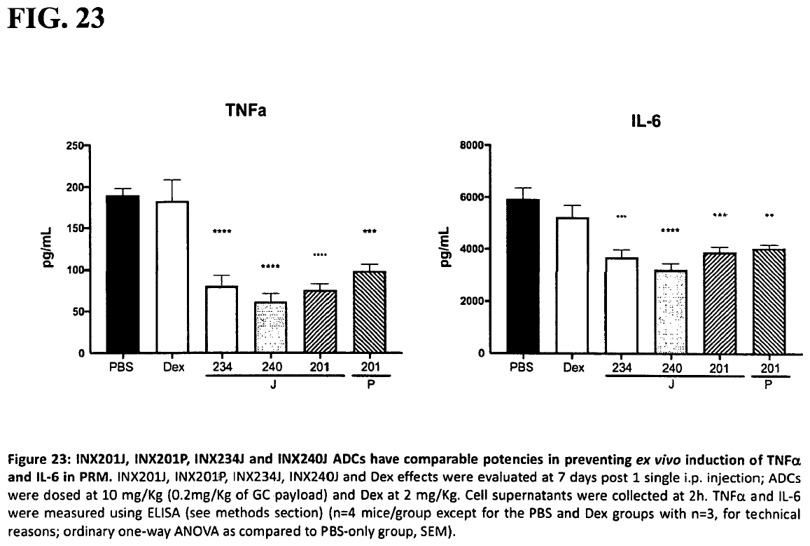

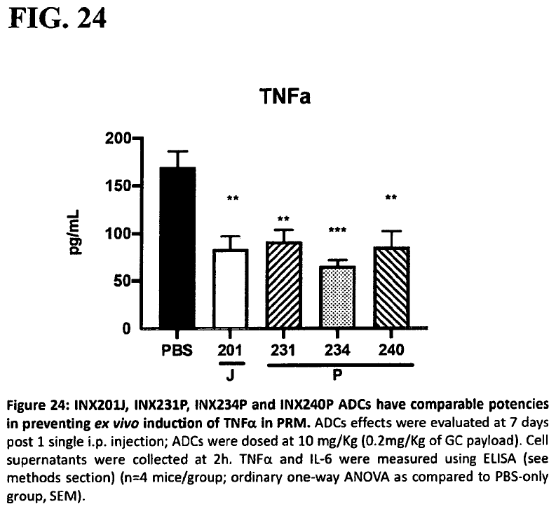

In this experiment in FIG. 24, the impact on extended potency of varying the anti-VISTA CDRs by assessing the INX P payload conjugated to different anti-VISTA antibodies as compared to INX201J was evaluated. All ADCs were injected i.p. on day −7, at a dosing of 0.2 mg/Kg of payload.

The data showed that all the anti-VISTA ADCs carrying the INX J or INX P payload have comparable potency after an extended period (FIG. 24). FIG. 24 shows INX201J, INX231P, INX234P and INX240P ADCs have comparable potencies in preventing ex vivo induction of TNFα in PRM. ADC effects were evaluated at 7 days post 1 single i.p. injection; ADCs were dosed at 10 mg/Kg (0.2 mg/Kg of GC payload). Cell supernatants were collected at 2h. TNFα was measured using ELISA (see methods section) (n=4 mice/group; ordinary one-way ANOVA as compared to PBS-only group, SEM).

### Experiment 8: Long-Term Potency of the INX R Linker Payload Conjugated to INX231

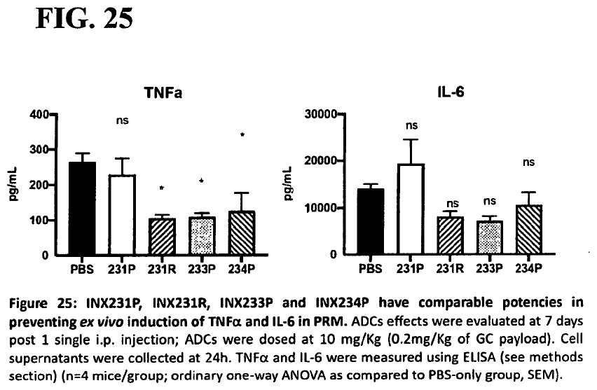

In this experiment in FIG. 25, the long-term potency of the INX R linker payload conjugated to INX231 was evaluated. This conjugate is analogous to INX231P; however, it contains a neutral dipeptide linker where INX231P has a negatively charged dipeptide linker. The potency of ADCs containing an additional anti-VISTA antibody INX233 was also evaluated. As comparators, INX231P and INX234P were used. All ADCs were injected i.p. on day −7, at a dosing of 0.2 mg/Kg of payload. Cell supernatants collected at 24h were analyzed as in previous experiments. No significant difference between supernatant collected at 2h or 24h was noted.

The data further showed that except for INX231P which showed lower than usual potency (see Experiment 6), INX231R and INX233P have comparable potency to INX234P (FIG. 25). Particularly, the data in FIG. 25 shows that INX231P, INX231R, INX233P and INX234P have comparable potencies in preventing ex vivo induction of TNFα and IL-6 in PRM. In the experiments the ADCs effects were evaluated at 7 days post 1 single i.p. injection; ADCs were dosed at 10 mg/Kg (0.2 mg/Kg of GC payload). Cell supernatants were collected at 24h. TNFα and IL-6 were measured using ELISA (see methods section) (n=4 mice/group; ordinary one-way ANOVA as compared to PBS-only group, SEM).

### Experiment 9: Long-Term Potency of the INX R Linker Payload Conjugated to INX231

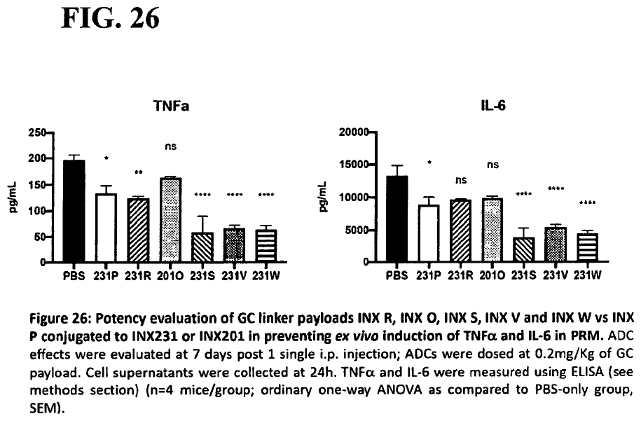

In this experiment in FIG. 26, we evaluated the long-term potency of several other INX linker payloads conjugated to INX231. In this experiment, the charge of the dipeptide linker (INX R, INX W vs INX P), the halogenation of the steroid ring (INX S vs INX P), and the payload (INX V vs INX P) were independently varied. The linker payload INX O that has a distinct payload from INX P was also evaluated as an INX201 conjugate. All ADCs were injected i.p. on day −7, at a dosing of 0.2 mg/Kg of payload. We analyzed cell supernatants collected at 24h.

As shown from the data in FIG. 26, the linker payloads INX S, INX V and INX W conjugated to INX231 showed remarkable long-term potency in controlling cytokine responses, while INX231P and INX231R displayed more limited though significant long-term efficacy; INX O payload conjugated to INX231 had little and non-significant impact on the PRM cytokine response FIG. 26 shows the potency evaluation of GC linker payloads INX R, INX O, INX S, INX V and INX W vs INX P conjugated to INX231 in preventing ex vivo induction of TNFα and IL-6 in PRM. ADCs effects were evaluated at 7 days post 1 single i.p. injection; ADCs were dosed at 0.2 mg/Kg of GC payload. Cell supernatants were collected at 24h. TNFα and IL-6 were measured using ELISA (see methods section) (n=4 mice/group; ordinary one-way ANOVA as compared to PBS-only group, SEM).

### Experiment 10: Potency of Payloads INX S, INX V and INX W at 1, 7 and 14 Days Pharmacodynamic Ranges

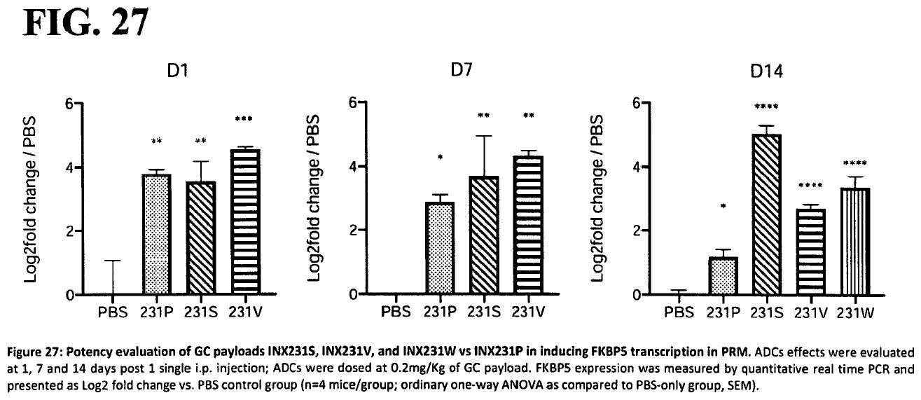

In this experiment in FIG. 27, the long-term potency of payloads INX S, INX V and INX W, looking at 1, 7 and 14 days pharmacodynamic ranges was evaluated. INX231P, INX231S and INX231V were injected i.p. on day −14, −7 or −1 and INX231W was injected on day −14 only. All ADCs were administered at a dosing of 0.2 mg/Kg of payload. PRM were collected on day 0. A fraction of the cells was used for RNA isolation while the other was put in culture. We analyzed cell supernatants collected at 24h post isolation.

As shown from the data in FIG. 27, after 24h (Day 1/D1) or 7 days, the payloads INX S and INX V display similar levels of induction of FKBP5 as INX P. After 14 days, while there is a clear decrease in FKBP5 transcription in the group treated with INX P, groups treated with payloads INX S, V and W still display high levels of FKBP5 transcripts. FIG. 27 contains the results of a potency evaluation of GC payloads INX231S, INX231V, and INX231W vs INX231P in inducing FKBP5 transcription in PRM. ADCs effects were evaluated at 1, 7 and 14 days post 1 single i.p. injection; ADCs were dosed at 0.2 mg/Kg of GC payload. FKBP5 expression was measured by quantitative real time PCR and presented as Log 2 fold change vs. PBS control group (n=4 mice/group; ordinary one-way ANOVA as compared to PBS-only group, SEM).

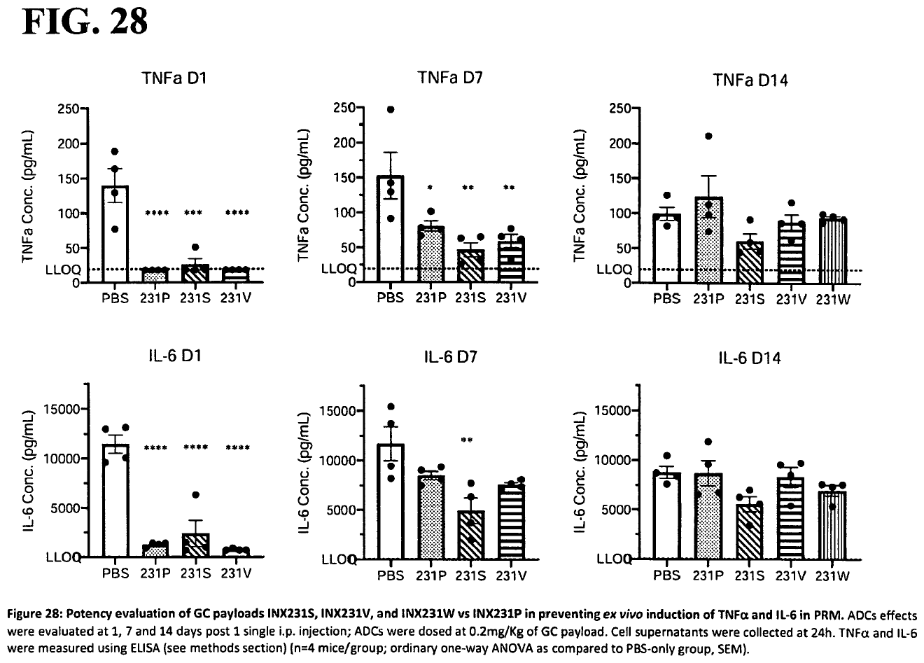

The experiment in FIG. 28 further shows that at 24h post treatment, payloads INX P, INX S and INX V have similar potency in preventing TNFα and IL-6 production by macrophages. Dosing 7 days earlier showed similar data with INX231S being slightly more potent. Dosing 14 days earlier led to a loss of functional activity of INX231P, INX231V and INX231W while INX231S retained some activity (the decrease in IL-6 on day 14 is close to significance p=0.0669). The data in FIG. 28 particularly shows a potency evaluation of GC payloads INX231S, INX231V, and INX231W vs INX231P in preventing ex vivo induction of TNFα and IL-6 in PRM. The ADCs effects were evaluated at 1, 7 and 14 days post 1 single i.p. injection; and ADCs were dosed at 0.2 mg/Kg of GC payload. Cell supernatants were collected at 24h. TNFα and IL-6 were measured using ELISA (see methods section) (n=4 mice/group; ordinary one-way ANOVA as compared to PBS-only group, SEM).

### Experiment 11: Potency of Conjugates INX234A3, INX234A4, INX234T, INX201L and INX231S Looking at 1, 7 and 14 Days

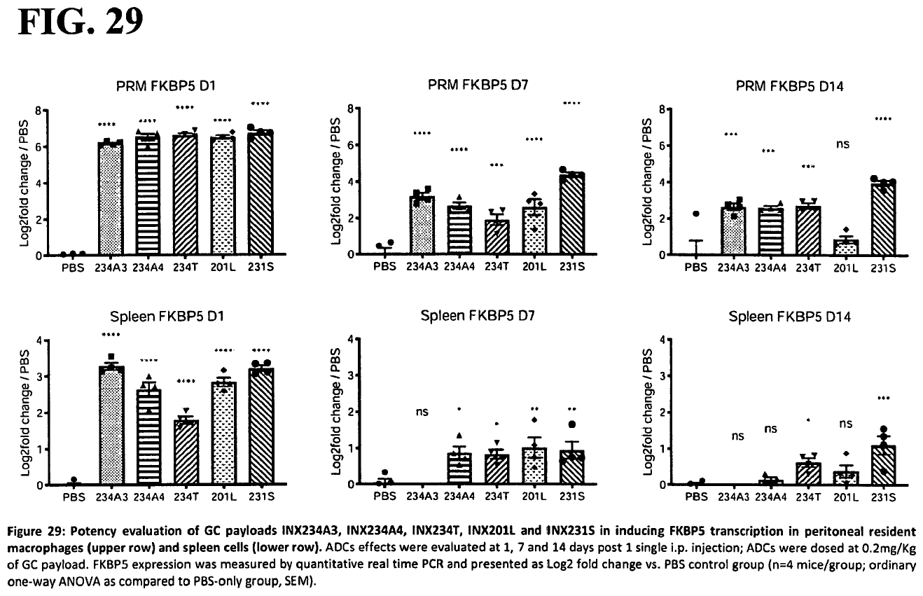

In this experiment in FIG. 29, the potency of 5 other payloads in the macrophage assay, INX234A3, INX234A4, INX234T, INX201L and INX231S, looking at 1, 7 and 14 days pharmacodynamic ranges was evaluated. All ADCs were injected i.p. on day −14, −7 or −1 at a dosing of 0.2 mg/Kg of payload. PRM were collected on day 0. A fraction of the cells was used for RNA isolation while the other was put in culture. Cell supernatants collected at 24h post isolation were analyzed. Isolated spleen cells were also collected and used to analyze FKBP5 expression levels.

The data in FIG. 29, show that all tested payloads induced similarly high levels of FKBP5 transcripts at 24 h. After 7 days, there is an overall decrease in FKBP5 transcription in all groups; by day 14, only the INX201L treated group shows continued FKBP5 decrease while INX234A3, INX234A4, INX234T and INX231S appear to induce stable FKBP5 transcription levels, unchanged from day 7.

In previous experiments, little to no FKBP5 signal in the spleen was observed when analyzed >4 days post injection with INX J and INX P payload conjugates. Very interestingly, the experiment in FIG. 29, which tested the potency of INX234A4, INX234T, INX201L and INX231S, showed significant induction of FKBP5 at day 7; moreover, by day 14, both INX234T and INX231S still induced significant increases. More specifically, FIG. 29 shows a potency evaluation of GC payloads INX234A3, INX234A4, INX234T, INX201L and INX231S in inducing FKBP5 transcription in peritoneal resident macrophages (upper row) and spleen cells (lower row). ADCs effects were evaluated at 1, 7 and 14 days post 1 single i.p. injection; ADCs were dosed at 0.2 mg/Kg of GC payload. FKBP5 expression was measured by quantitative real time PCR and presented as Log 2 fold change vs. PBS control group (n=4 mice/group; ordinary one-way ANOVA as compared to PBS-only group, SEM).

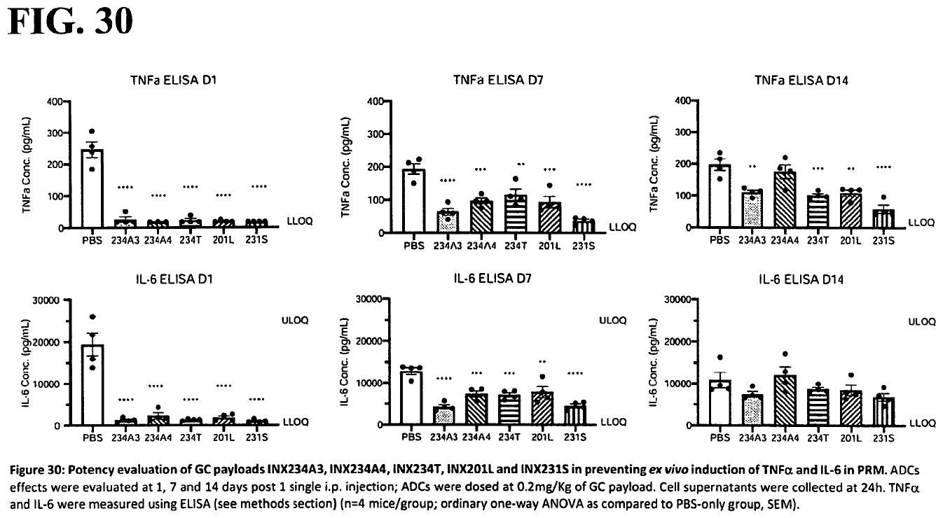

The experiment in FIG. 30 further shows that at 24h post treatment, all payloads INX A3, INX A4, INX T, INX L and INX S have similar potency in preventing TNFα and IL-6 production by macrophages. Dosing 7 days earlier showed similar results with INX234A3 with INX231S being slightly more potent. Dosing 14 days earlier led to a loss of functional activity of INX234A4 in controlling both TNFα and IL-6 production but interestingly, INX234A3, INX234T, INX201L and INX231S still significantly reduced TNFα production. More particularly, the experiment in FIG. 30 evaluates the potency of GC payloads INX234A3, INX234A4, INX234T, INX201L and INX231S in preventing ex vivo induction of TNFα and IL-6 in PRM. These ADCs effects were evaluated at 1, 7 and 14 days post 1 single i.p. injection; ADCs were dosed at 0.2 mg/Kg of GC payload. Cell supernatants were collected at 24h. TNFα and IL-6 were measured using ELISA (see methods section) (n=4 mice/group; ordinary one-way ANOVA as compared to PBS-only group, SEM).

### Experiment 12: Potency of Conjugates INX234A5 and INX234A11 vs INX234V at 7 and 14 Days

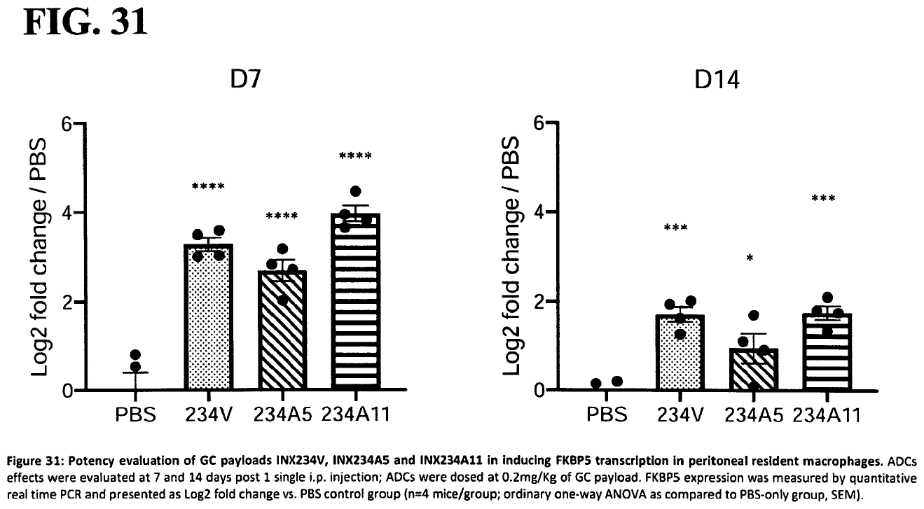

In this experiment in FIG. 31, the potency of 2 other payloads in the macrophage assay, i.e., INX234A5 and INX234A11 vs INX234V looking at 7 and 14 days pharmacodynamic ranges was evaluated. All ADCs were injected i.p. on day 0 at a dosing of 0.2 mg/Kg of payload. PRM were collected on days 7 and 14. A fraction of the cells was used for RNA isolation while the other was put in culture. Cell supernatants collected at 24h post isolation were evaluated. As shown from the data in FIG. 31, all payloads induced similarly high levels of FKBP5 transcripts 7 days post injection. By day 14, there is an overall decrease in FKBP5 transcription but with still significant differences as compared to the control group.

More specifically, in the experiment in FIG. 31 the potency of GC payloads INX234V, INX234A5 and was evaluated in inducing FKBP5 transcription in peritoneal resident macrophages was assayed wherein ADCs effects were evaluated at 7 and 14 days post 1 single i.p. injection; and wherein these ADCs were dosed at 0.2 mg/Kg of GC payload. FKBP5 expression was measured by quantitative real time PCR and presented as Log 2 fold change vs. PBS control group (n=4 mice/group; ordinary one-way ANOVA as compared to PBS-only group, SEM).

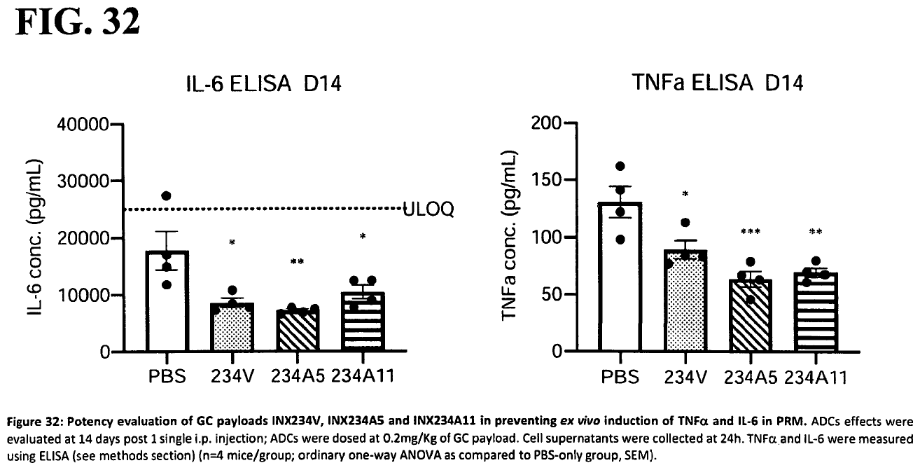

The experiment in FIG. 32 further shows that after 14 days, all of the tested ADCs have retained similar potency in preventing TNFα and IL-6 production by macrophages. Particularly, the experiment in FIG. 32 compares the potency of GC payloads INX234V, INX234A5 and INX234A11 in preventing ex vivo induction of TNFα and IL-6 in PRM. The effects of these ADCs were evaluated at 14 days post 1 single i.p. injection; and the ADCs were dosed at 0.2 mg/Kg of GC payload. Cell supernatants were collected at 24h. TNFα and IL-6 were measured using ELISA (see methods section) (n=4 mice/group; ordinary one-way ANOVA as compared to PBS-only group, SEM).

### Experiment 13: Potency of Conjugates INX231A7, INX231A12 and INX231A23 vs INX234V at 7, 14 and 21 Days

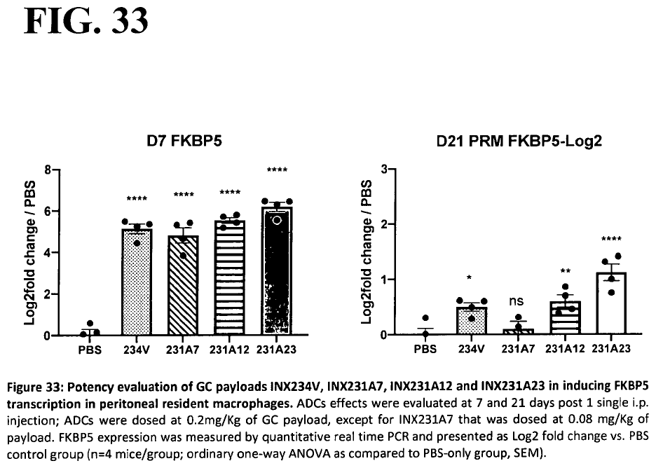

In this experiment in FIG. 33, the potency of 3 other payloads in the macrophage assay, INX231A7, INX231A12 and INX231A23 vs INX234V looking at 7 days and 21 days post injection pharmacodynamic ranges was evaluated. All ADCs were injected i.p. on day 0 at a dosing of 0.2 mg/Kg of payload, except for INX231A7 that was dosed at 0.08 mg/Kg of payload. PRM were collected on day 7. A fraction of the cells was used for RNA isolation while the other was put in culture. Cell supernatants collected at 24h post isolation were evaluated. More specifically in these experiments in FIG. 33 the potency of GC payloads INX234V, INX231A7, INX231A12 and INX231A23 in inducing FKBP5 transcription in peritoneal resident macrophages was assessed wherein ADCs effects were evaluated at 7 and 21 days post 1 single i.p. injection; ADCs were dosed at 0.2 mg/Kg of GC payload, except for INX231A7 that was dosed at 0.08 mg/Kg of payload. FKBP5 expression was measured by quantitative real time PCR and presented as Log 2 fold change vs. PBS control group (n=4 mice/group; ordinary one-way ANOVA as compared to PBS-only group, SEM).

As shown from the data in FIG. 33, all of the tested payloads induced similarly high levels of FKBP5 transcripts 7 days post injection, with INX231A23 being slightly more potent. By day 21 post injection, small but significant increases in FKBP5 transcription compared to the PBS treated group were still detected, except for the INX231A7 treated group.

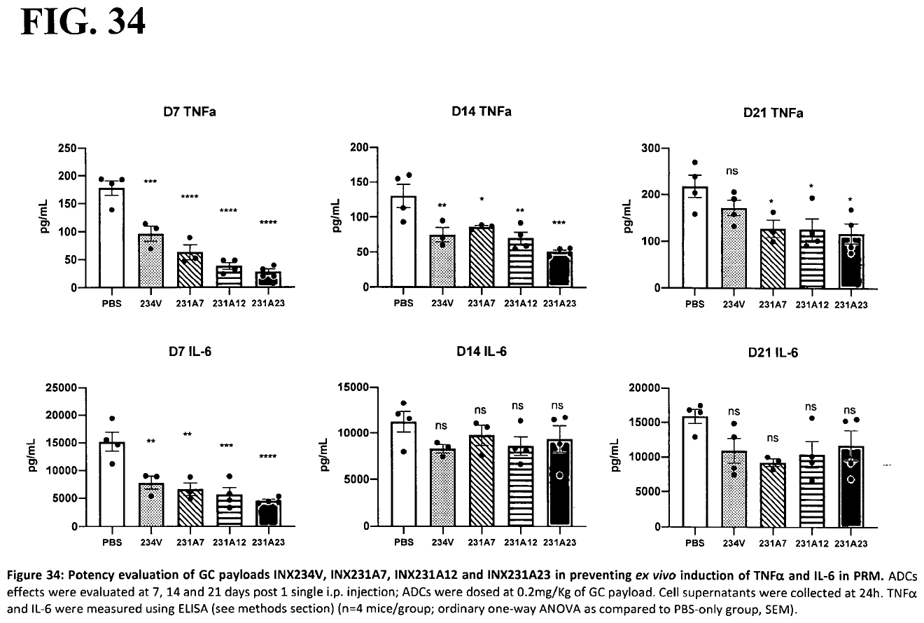

The results of the experiment which are contained in FIG. 34 further show that after 7, 14 and 21 days, all of the tested ADCs have retained similar potency in preventing TNFα and IL-6 production by macrophages, with again INX231A23 being the most efficacious particularly in regard to TNFα production. At 14 days post injection, significant reductions in TNFα were observed for all ADCs tested and by 21 days post dosing, there were still significant decreases in TNFα production for groups treated with INX231A7, INX231A12 and INX231A23.

More specifically, FIG. 34 shows the results of a potency evaluation of GC payloads INX234V, INX231A7, INX231A12 and INX231A23 in preventing ex vivo induction of TNFα and IL-6 in PRM. In these experiments ADCs effects were evaluated at 7, 14 and 21 days post 1 single i.p. injection; ADCs were dosed at 0.2 mg/Kg of GC payload. Cell supernatants were collected at 24h. TNFα and IL-6 were measured using ELISA (see methods section) (n=4 mice/group; ordinary one-way ANOVA as compared to PBS-only group, SEM). Of particular note is the fact that INX231A7 while only dosed at 0.08 mg/Kg of payload, it still had significant impact on TNFα production after 21 days.

### Conclusions

The data show that:
A single injection of INX201J induces long-term transcriptional induction of GC reporter transcript FKBP5 in the PRM target cell population demonstrating that the ADC has a pharmacodynamic range >4 days, whereas FKBP5 transcriptional induction by Dex is undetectable by 24h.
The linker payloads INX S, INX V, INX W, INX A3, INX A4, INX A5, INX A11 and INX T still display significant potency when dosed 14 days early in regard to FKBP5 induction.
Significant FKBP5 induction is seen at day 1, 7, and 14 in splenic cells with analogs of INX P, specifically INX T (phosphorylated) and INX S (fluorinated at C6/C9). INX A4, as well as the literature precedent, INX L (the phosphorylated version of INX J) only demonstrate significant induction at day 1, and 7.

In our model of in vivo treatment/ex vivo evaluation of PRM inflammation status based on pro-inflammatory IL-6 and TNFα production/secretion:
The ADCs tested (INX231J/INX234J/INX240J) display potency after 7 days even when dosed at 0.02 mg/Kg of payload, which is 100-fold less than the Dex control dosed at 2 mg/Kg.
All the ADCs tested (INX201J/INX201P/INX231J/INX234J/INX240J) have a pharmacological range >7 days in PRM based on TNFα and IL-6 reductions whereas Dex has lost all potency.
The linker/payloads INX R, INX P and INX J show similar potency when dosed 7 days early.
The linker/payloads INX S, INX V and INX W appear remarkably more potent than payloads INX P and INX R when dosed 7 days early. For INX V and INX W, this is potentially due to enhanced release. For INX S, this is likely due to the potency of the free payload which is fluorinated at C6/C9.
The linker payload INX O shows very limited and non-significant potency when conjugated to INX201.
The linker/payloads INX S, INX V, INX A3, INX T, INX L, INX A5 and INX A11 had significant potency in controlling cytokine response in PRM when dosed 14 days early.
The linker/payloads INX A7, INX A23 and INX A12 elicit potency at least 21 days post injection.

Of particular note in these assays—all analogs of INX V containing the spiro[3.3]heptane were potent for at least 7 days post-injection based on their ability to elicit FKBP5 and cytokine reduction in the ex vivo PRM model. The tested analogs shown to be potent for at least 7 days include INX A3, INX A4, INX A5, INX A7, INX A11, INX A12 and INX A23 and additionally include the following:
Phosphorylated analogs (INX A12/INX A7)
Doubly fluorinated analogs (INX A23/A12)
Ether variation of the methylene between the phenyl ring and the spiro[3.3]heptane (INX A4 and INX A5)
Linker variants of the negatively charged gly/glu linker including positively charged lys/gly (INX A3), negatively charged Asn/gly (INX A11)

### Example 7: Impact of Antibody Drug Conjugates on LPS Induced Inflammation

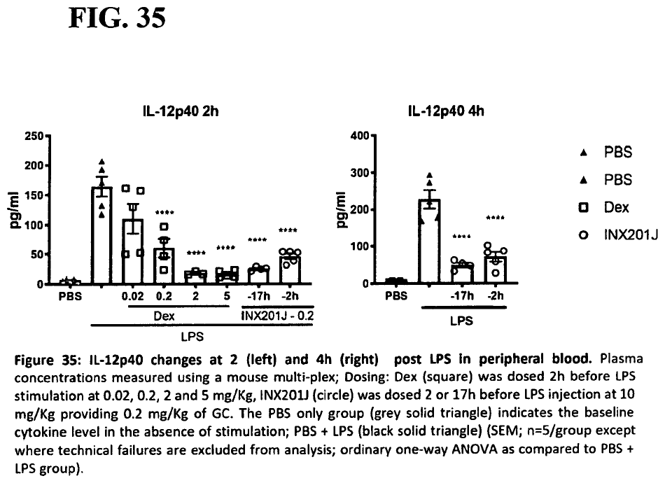

Fourteen in vivo studies the results of which are disclosed in this example and shown in FIGS. 35-48 were conducted to assess the impact of exemplary antibody drug conjugates according to the invention on LPS-induced inflammation.

In order to evaluate the ADC potential efficacy in auto-immune diseases, we used the short-term model of LPS-induced systemic inflammation. Intraperitoneal (i.p.) injection of lipopolysaccharide (LPS) is widely used as a model for acute immune response-both local and systemic—in mice. The LPS model is characterized by a burst of pro-inflammatory cytokines in the blood circulation that can be monitored as early as 2h post injection. By 24h, most cytokines are back to normal levels. We took advantage of this model by mainly monitoring cytokine response at 2 or 4h post LPS injection. Preliminary studies showed that Dexamethasone (Dex) treatment has a dose dependent effect on IL-12p40, TNFα, MIG, MIP-1a, and IL-1β detectable as early as 2h, so our studies focused on measuring one or more of these 5 cytokines. (See Vermeer et al. (2003) Glucocorticoid-induced increase in lymphocytic FKBP51 messenger ribonucleic acid expression: a potential marker for glucocorticoid sensitivity, potency, and bioavailability. J Clin Endocrinol Metab. January; 88 (1): 277-84)

The objective of the studies was to evaluate the efficacy of human anti-VISTA antibodies conjugated to various glucocorticoid payloads as compared to free Dex.

### Materials and Methods

### Methods

In these experiments, mice received antibody or Dex treatments at ˜20h or 2-4h, respectively, before LPS injection. Dex is short-lived and acts quickly, whereas the ADC requires additional processing time. These time points were chosen as a way to fairly compare peak activities of ADC and Dex.

Blood was collected at 2 or 4h post LPS i.p. injection, and plasma isolated for cytokine analyses.

### Test Agents and Dosage

### Antibodies

INX201 (Aragen, Lot #BP-3200-019-6) is a humanized anti-human VISTA antibody on a human IgG1/kappa backbone with L234A/L235A/E269R/K322A silencing mutations in the Fc region.
huIgG1si (Aragen, Lot #BP-2211-018-6) is an anti-RSV mAb on a human IgG1/kappa backbone with E269R/K322A silencing mutations in the Fc region.
INX201J (Abzena, Lot #s JZ-0556-025-1, JZ-0556-027, JZ-0556-013) is the INX201 antibody with a drug/antibody ratio (DAR) of 8.0, conjugated via full modification of the interchain disulfides. The linker/payload (J) is based on a previously reported linker/payload (U.S. Ser. No. 15/611,037). It consists of a negatively charged protease sensitive linker with a budesonide analog payload (INX J-2).
huIgG1si J (Abzena, Lot #JZ-0556-025-2) is the huIgG1si antibody with a DAR of 8.0, conjugated via full modification of the interchain disulfides with the INX J linker/payload.
INX201N (Abzena, Lot #JZ-0556-028) is INX201 with a DAR of 8.0, conjugated via full modification of the interchain disulfides. The linker/payload (INX N) consists of a negatively charged protease sensitive linker with a budesonide analog payload (INX-SM-1).
INX201O (Abzena, Lot #JZ-0556-016-2) is INX201 with a DAR of 8.0, conjugated via full modification of the interchain disulfides. The linker/payload (INX O) consists of a negatively charged protease sensitive linker with a budesonide analog payload (INX-SM-4).
INX201P (Abzena, Lot #JZ-0556-016-1) is INX201 with a DAR of 8.0, conjugated via full modification of the interchain disulfides. The linker/payload (INX P) consists of a negatively charged protease sensitive linker with a budesonide analog payload (INX-SM-3).
INX233 (ATUM Lot #82276.1.a) is a humanized anti-human VISTA antibody on a human IgG1/kappa backbone with L234A/L235A/E269R/K322A silencing mutations in the Fc region.
INX233P (Abzena, Lot #PP-0924-001-3) is INX233 with a DAR of 8.0, conjugated via full modification of the interchain disulfides. The linker/payload (INX P) consists of a negatively charged protease sensitive linker with a budesonide analog payload (INX-SM-3).
INX231 (ATUM Lot #72928.1.a) is a humanized anti-human VISTA antibody on a human IgG1/kappa backbone with L234A/L235A/E269R/K322A silencing mutations in the Fc region.
INX231P (Abzena, Lot #JZ-0556-017-1) is INX201 with a DAR of 8.0, conjugated via full modification of the interchain disulfides. The linker/payload (INX P) consists of a negatively charged protease sensitive linker with a budesonide analog payload (INX-SM-3).
INX231R (Abzena, Lot #PP-0924-001-2) is INX231 with a DAR of 8.0, conjugated via full modification of the interchain disulfides. The linker/payload (INX R) consists of a neutral protease sensitive linker with a budesonide analog payload (INX-SM-3).
INX231S (Abzena, lot #PP-0920-014-1) is INX231 with a DAR of 6.9, conjugated via modification of the interchain disulfides. The linker/payload (INX S) consists of a negatively charged protease sensitive linker with a fluocinolone acetonide analog payload (INX-SM-24).
INX231V (Abzena, lot #PP-0920-014-2) is INX231 with a DAR of 7.8, conjugated via modification of the interchain disulfides. The linker/payload (INX V) consists of a negatively charged protease sensitive linker with a budesonide analog payload (INX-SM-32).
INX231W (Abzena, lot #PP-0920-014-3) is INX231 with a DAR of 7.5, conjugated via modification of the interchain disulfides. The linker/payload (INX W) consists of a positively charged protease sensitive linker with a budesonide analog payload (INX-SM-3).
INX231J (Abzena, Lot #JZ-0556-013-1) is the INX231 antibody with a drug/antibody ratio (DAR) of 8.0, conjugated via full modification of the interchain disulfides. The linker/payload (J) consists of a negatively charged protease sensitive linker with a budesonide analog payload (INX J-2).
INX234J (Abzena, Lot #JZ-0556-013-3) is the INX234 antibody with a drug/antibody ratio (DAR) of 8.0, conjugated via full modification of the interchain disulfides. The linker/payload (J) consists of a negatively charged protease sensitive linker with a budesonide analog payload (INX J-2).
INX240J (Abzena, Lot #JZ-0556-013-3) is the INX240 antibody with a drug/antibody ratio (DAR) of 8.0, conjugated via full modification of the interchain disulfides. The linker/payload (J) consists of a negatively charged protease sensitive linker with a budesonide analog payload (INX J-2).
INX234P (Abzena, Lot #HA-0853-02) is INX234 with a DAR of 8.0, conjugated via full modification of the interchain disulfides. The linker/payload (INX P) consists of a negatively charged protease sensitive linker with a budesonide analog payload (INX-SM-3).
INX234A3 (Abzena, Lot #PP-0924-023-1) is the INX234 antibody with a drug/antibody ratio (DAR) of 8.0, conjugated via full modification of the interchain disulfides. The linker/payload (INX A3) consists of a positively charged protease sensitive linker with a budesonide analog payload (INX-SM-32).
INX234A4 (Abzena, Lot #PP-0924-023-2) is the INX234 antibody with a drug/antibody ratio (DAR) of 7.9, conjugated via full modification of the interchain disulfides. The linker/payload (INX A4) consists of a negatively charged protease sensitive linker with a budesonide analog payload (INX-SM-43).
INX234T (Abzena, Lot #PP-0924-023-3) is the INX234 antibody with a drug/antibody ratio (DAR) of 8.0, conjugated via full modification of the interchain disulfides. The linker/payload (INX T) consists of a negatively charged protease sensitive linker with a budesonide analog payload (INX-SM-3) that is phosphorylated.
INX234V (Abzena, RJS-1054-003) is the INX234 antibody with a drug/antibody ratio (DAR) of 8.0, conjugated via full modification of the interchain disulfides. The linker/payload (INX V) consists of a negatively charged protease sensitive linker with a budesonide analog payload (INX-SM-32).
INX234A5 (Abzena, Lot #RJS-1054-002) is the INX234 antibody with a drug/antibody ratio (DAR) of 7.9, conjugated via full modification of the interchain disulfides. The linker/payload (INX A5) consists of a negatively charged protease sensitive linker with a budesonide analog payload (INX-SM-44).
INX234A11 (Abzena, Lot #RJS-1054-001) is the INX234 antibody with a drug/antibody ratio (DAR) of 8.0, conjugated via full modification of the interchain disulfides. The linker/payload (INX A11) consists of a negatively charged protease sensitive linker (Asn/gly) with a budesonide analog payload (INX-SM-32).
INX231A7 (Abzena, Lot #RJS-1054-007-001) is the INX234 antibody with a drug/antibody ratio (DAR) of 7.8, conjugated via full modification of the interchain disulfides. The linker/payload (INX A7) consists of a negatively charged protease sensitive linker with a budesonide analog payload (INX-SM-32) that is phosphorylated.
INX231A12 (Abzena, Lot #RJS-1054-007-002) is the INX234 antibody with a drug/antibody ratio (DAR) of 6.99, conjugated via full modification of the interchain disulfides. The linker/payload (INX A12) consists of a negatively charged protease sensitive linker with a fluocinolone acetonide analog payload (INX-SM-25) that is phosphorylated.
INX231A23 (Abzena, Lot #RJS-1054-006-001) is the INX234 antibody with a drug/antibody ratio (DAR) of 7.34, conjugated via full modification of the interchain disulfides. The linker/payload (INX A23) consists of a negatively charged protease sensitive linker with a fluocinolone acetonide analog payload (INX-SM-25).
INX234A13 (Abzena, Lot #RJS-1054-007-003) is the INX234 antibody with a drug/antibody ratio (DAR) of 5.82, conjugated via full modification of the interchain disulfides. The linker/payload (INX A13) consists of a negatively charged protease sensitive linker with a budesonide analog payload (INX-SM-34).
INX234A1 (Abzena, Lot #RJS-1054-004) is the INX234 antibody with a drug/antibody ratio (DAR) of 7.6, conjugated via full modification of the interchain disulfides. The linker/payload (INX A1) consists of a negatively charged protease sensitive linker with a budesonide analog payload (INX-SM-35).
Target B—P (Abzena PP-0924-031) is an anti-mouse Target B antibody with a DAR of 8.0, conjugated via full modification of the interchain disulfides. This antibody binds to an immune cell specific antigen other than VISTA, which is expressed on different immune cell types and is an internalizing antibody. The linker/payload (INX P) consists of a negatively charged protease sensitive linker with a budesonide analog payload (INX-SM-3).
INX201V (Abzena, lot #SCG-1120-012-1) is INX201 with a DAR of 7.86, conjugated via full modification of the interchain disulfides. The linker/payload (INX V) consists of a negatively charged protease sensitive linker with a budesonide analog payload (INX-SM-32).
INX234A9 (Abzena, lot #AF-1114-007-1) is the INX234 antibody with a drug/antibody ratio (DAR) of 7.3, conjugated via modification of the interchain disulfides. The linker/payload (INX A9) consists of a negatively charged protease sensitive linker with a budesonide analog payload (INX-SM-46).

The antibodies were diluted in PBS and injected intraperitoneal (i.p.) in a volume of 0.2 ml to deliver a specified dose.

### Dexamethasone

Dexamethasone sterile injection from Phoenix, NDC 57319-519-05, was diluted in PBS and dosed as described via i.p. injection.

### LPS

LPS was obtained from AMSBIO (#9028). Mice were dosed at 0.5 mg/Kg.

### Mice

The hVISTA mice were bred on site (Center for Comparative Medicine and Research at Dartmouth). All the experiments were done in female mice enrolled between 9 and 15 weeks of age.

### Blood Draw and Preparation

Peripheral blood was harvested from the retro-orbital cavity using a glass Pasteur pipette that was first rinsed with heparin to prevent coagulation. Blood was then centrifuged at 550 rcf for 5 min and plasma collected and stored at −80° C. before cytokine analysis.

### Plasma Cytokine Analysis

### Cytokine Analyses Using a Millipore Platform

Cytokine analyses were conducted on 25 μl of plasma using a Millipore mouse 5 or 7-plex platform. For ADC-INVIVO-30 and 35, the Immune Monitoring Lab (IML, Shared Resources at Dartmouth-Hitchcock Norris Cotton Cancer Center) performed the analyses.

Cytokines included in the analysis were MIP-1a, TNFα, IL-1β, IL-12p40 and MIG and were detected via ELISA as follows:
BioLegend (cat #430904) ELISA MAX Deluxe Set Mouse TNF-α
BioLegend (cat #431604) ELISA MAX Deluxe Set Mouse IL-12/IL-23 (p40)

ELISAs were conducted following the manufacturer's included protocol.

Cytokine data are censored when below a 20 μg/ml threshold for IL-12p40 and/or 10 μg/ml threshold for TNF-α as it indicates a failed LPS injection.

### Cell Isolation

After euthanasia, mice were injected in the peritoneal cavity with 7 ml of PBS/0.5% BSA/2 mM EDTA. After a brief massage of the peritoneum, a small incision was performed and the peritoneal lavage collected. PRM were isolated using negative selection (Miltenyi kit, ref 130-110-434). Spleen were dissected and dissociated mechanically; monocytes were isolated using negative selection (Stem Cell, EasySep™ Mouse CD11b Positive Selection Kit II).

### RNA Preparation and Real Time PCR

Cell pellets from different tissues were resuspended in 0.4 ml RNeasy lysis buffer from RNeasy Plus Mini kit (Qiagen, PN: 74136) and homogenized with 20G needle for 5 times. RNA was isolated following manufacturer's instructions and eluted in 30 or 40 μl H₂O (RNase/DNase free). RNA concentration was assessed by UV spectroscopy using a Nanodrop.

Reverse transcription was done using Taqman reverse transcription reagents (#N8080234) and following manufacturer's instructions.

Quantitative Real-Time PCR was done using Taqman master mix 2× kit (#4369016) and Taqman primers for mouse FKBP5 (Mm00487401_m1), and mouse HPRT as housekeeping gene (Mm446968_m1) and run on a QuantStudio3 from Applied Biosystem.

Ct data were converted to ΔCt (FKBP5 normalized to HPRT within a sample) and then ΔΔCt (FKBP5 relative levels for treated sample vs PBS control) to obtain Log 2 fold-changes relative to PBS.

### Results

### Experiment 1: Evaluation of In Vivo Efficacy of INX201J Efficacy in LPS-Induced Cytokine Release

As shown in FIG. 35, treatment with INX201J at 10 mg/Kg showed similar efficacy to Dex at 2 mg/Kg in controlling LPS-induced IL-12p40 release. It is important to note that INX201J at 10 mg/kg delivers the molar equivalent of 0.2 mg/Kg of GC payload, the dose at which Dex had only partial efficacy.

In this experiment, we also evaluated if our ADC needed more time for processing than free Dex. We showed that efficacy was improved when the ADC was dosed 17h before LPS injection when compared to 2h pre LPS. No difference in cytokine response was noted between 2 and 4h post LPS and for all our next studies, we collected blood plasma only at 2h timepoint. FIG. 35 shows IL-12p40 changes at 2 (left) and 4h (right) post LPS in peripheral blood. Plasma concentrations measured using a mouse multi-plex; Dosing: Dex (square) was dosed 2h before LPS stimulation at 0.02, 0.2, 2 and 5 mg/Kg, INX201J (circle) was dosed 2 or 17h before LPS injection at 10 mg/Kg providing 0.2 mg/Kg of GC. The PBS only group (grey solid triangle) indicates the baseline cytokine level in the absence of stimulation; PBS+LPS (black solid triangle) (SEM; n=5/group except where technical failures are excluded from analysis; ordinary one-way ANOVA as compared to PBS+LPS group). The data show that INX201J dosed at 10 mg/Kg (delivering ˜0.2 mg/Kg of GC payload) showed similar efficacy to dexamethasone dosed at 2 or 5 mg/Kg in controlling the LPS induced IL-12p40 response.

### Experiment 2: INX201J Dose Response in LPS-Induced Cytokine Release

In this experiment, we evaluated INX201J anti-inflammatory properties at higher dilutions. As shown in FIG. 36, INX201J at 10 mg/Kg (0.2 mg/Kg of payload) had equivalent efficacy as Dex at 2 mg/Kg efficacy for all the cytokines analyzed except MIG, while Dex at 0.2 mg/Kg has reduced efficacy compared to INX201J. INX201J still showed some efficacy when diluted at 0.06 and 0.02 mg/Kg of payload.

We also tested the efficacy of Dex when injected at 17h pre LPS, as was done with INX201J. More specifically, the experimental data in FIG. 36 shows cytokine changes at 2h post LPS in peripheral blood. In these experiments plasma concentrations were measured using a mouse 5-plex; Dex was dosed 2h before LPS stimulation at 0.002, 0.02, 0.2, 2 mg/Kg (square) or at 2 mg/Kg 17h pre LPS (black solid square), and INX201J (circle) was dosed 17h before LPS injection at 0.02, 0.06, 0.2 mg/Kg of GC payload. The PBS only group (solid grey triangle) indicates the baseline cytokine level in the absence of stimulation; PBS+LPS (solid black triangle) (SEM; n=5/group, except where technical failures are excluded from analysis; ordinary one-way ANOVA as compared to PBS+LPS group). As expected, due the short half-life of Dex, there was a loss in potency in that group when compared to the group dosed 2h pre LPS, suggesting that INX201J may have increased pharmacodynamic impact on cytokine production. The data shows that INX201J had comparable efficacy at 10 mg/Kg (delivering ˜0.2 mg/Kg of GC payload) to Dex at 2 mg/Kg in controlling the LPS-induced MIP-1a, TNFα, IL-1β, IL-12p40 and MIG responses. Comparatively, Dex at 0.2 mg/Kg had only limited efficacy.

### Experiment 3: INX201J Dose Response in LPS-Induced Cytokine Release

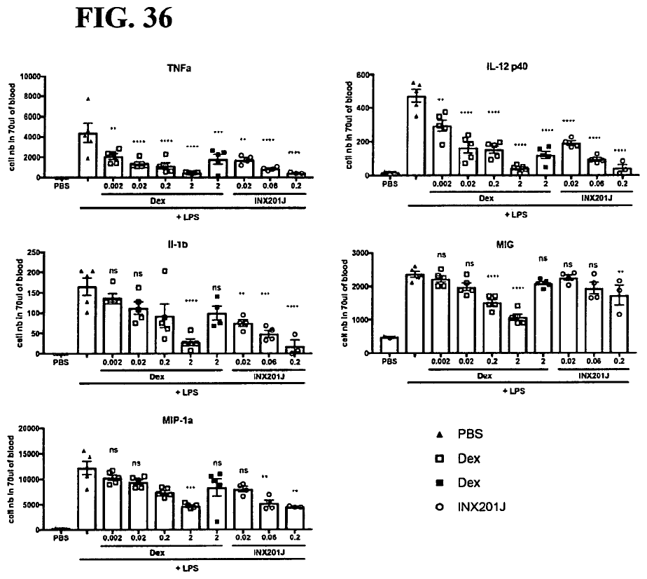

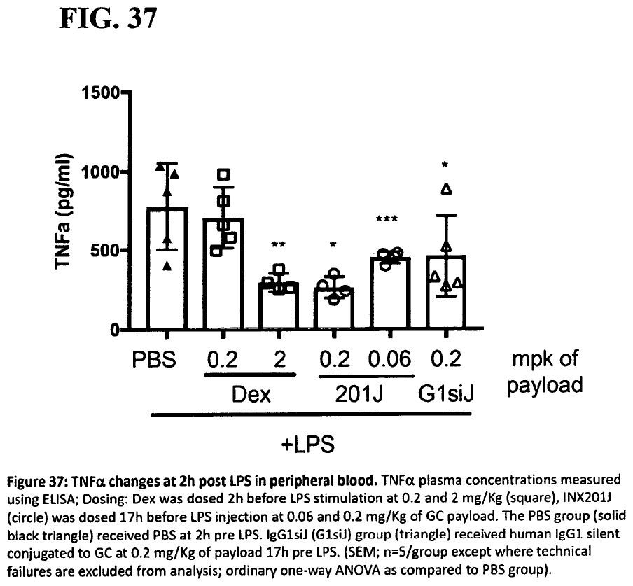

In this experiment, the efficacy of INX201J at 0.2 and 0.06 mg/Kg of GC payload to free Dex at 2 and 0.2 mg/Kg was compared. Specifically, the experiment in FIG. 37 shows TNFα changes at 2h post LPS in peripheral blood. TNFα plasma concentrations measured using ELISA; Dosing: Dex was dosed 2h before LPS stimulation at 0.2 and 2 mg/Kg (square), INX201J (circle) was dosed 17h before LPS injection at 0.06 and 0.2 mg/Kg of GC payload. The PBS group (solid black triangle) received PBS at 2h pre LPS. IgG1siJ (G1siJ) group (triangle) received human IgG1 silent conjugated to GC at 0.2 mg/Kg of payload 17h pre LPS. (SEM; n=5/group except where technical failures are excluded from analysis; ordinary one-way ANOVA as compared to PBS group).

As shown from the data in FIG. 37, INX201J at 0.2 mg/Kg of GC payload showed comparable efficacy in controlling TNFα response to LPS to Dex at 2 mg/Kg. INX201J at 0.06 mg/Kg of GC payload still displayed higher efficacy than Dex at 0.2 mg/Kg. The control group injected with the control human IgG1 silent conjugated to the same payload showed some level of efficacy in preventing TNFα up-regulation. The dose response study showed improved/boost in efficacy when a GC payload was delivered via the ADC INX201J: while free Dex at 0.2 mg/Kg showed no efficacy in preventing the LPS induced up-regulation of TNFα, the molar equivalent of a GC payload delivered via ADC showed high potency. To note: the human IgG1 silent control conjugated to GC (same linker and GC payload as INX201J) appeared to weakly impact LPS induced up-regulation of TNFα.

### Experiment 4: Evaluation of In Vivo Efficacy of INX201J Vs. Dexamethasone in LPS-Induced Cytokine Release

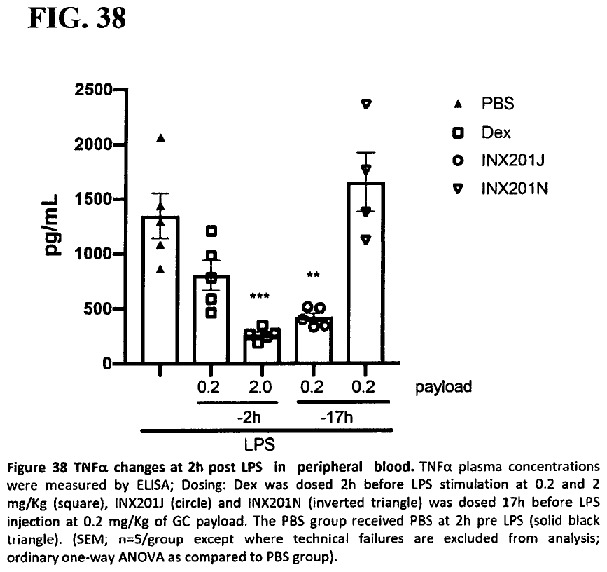

The experiment in FIG. 38 shows TNFα changes at 2h post LPS in peripheral blood with different ADCs wherein TNFα plasma concentrations were measured by ELISA; Dex was dosed 2h before LPS stimulation at 0.2 and 2 mg/Kg (square), INX201J (circle) and INX201N (inverted triangle) were each dosed 17h before LPS injection at 0.2 mg/Kg of GC payload; and the PBS group received PBS at 2h pre LPS (solid black triangle). (SEM; n=5/group except where technical failures are excluded from analysis; ordinary one-way ANOVA as compared to PBS group).

As shown from the data in FIG. 38 and as further observed in experiments above, INX201J at 0.2 mg/Kg of GC payload had similar efficacy to Dex at 2 mg/Kg in controlling LPS induced cytokine response. Additionally, the data shows that INX201N had no efficacy in controlling the TNFα response, possibly due to inefficient cleavage or microaggregate formation of the released product of this linker/payload in vivo. INX201J prevented LPS induced up-regulation of TNFα when dosed at 10 mg/Kg (˜0.2 mg/Kg of GC payload) with equivalent potency as Dex at 2 mg/Kg. Conversely, INX201N showed no efficacy in that model.

### Experiment 5: Evaluation of In Vivo Efficacy of INX231J, INX234J and INX240J Vs. INX201J in LPS-Induced Cytokine Release

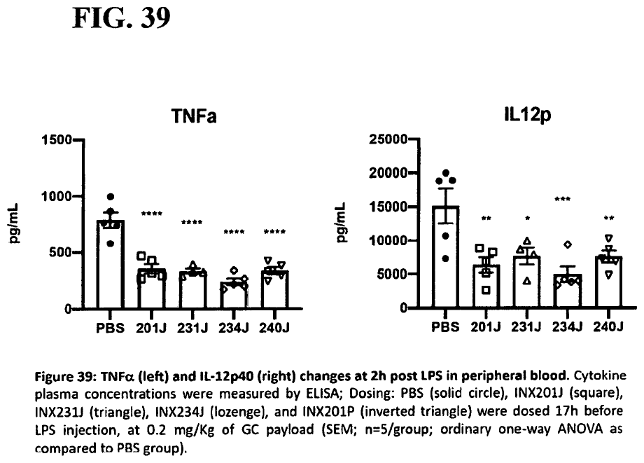

We further evaluated 3 different anti-VISTA antibodies conjugated to the same INX J payload. Specifically the experiment in FIG. 39 shows TNFα (left) and IL-12p40 (right) changes at 2h post LPS in peripheral blood wherein cytokine plasma concentrations were measured by ELISA; and PBS (solid circle), INX201J (square), INX231J (triangle), INX234J (lozenge), and INX201P (inverted triangle) were dosed 17h before LPS injection, at 0.2 mg/Kg of GC payload (SEM; n=5/group; ordinary one-way ANOVA as compared to PBS group).

As shown from the data in FIG. 39, INX231J, INX234J and INX240J showed similar potency to INX201J in controlling LPS-induced cytokine responses. All ADCs dosed at 10 mg/Kg (delivering ˜0.2 mg/Kg of GC payload) showed comparable efficacy to INX201J in controlling the LPS-induced TNFα and IL-12p40 responses.

### Experiment 6: Evaluation of In Vivo Efficacy of INX201O and INX201P Vs. INX201J in LPS-Induced Cytokine Release

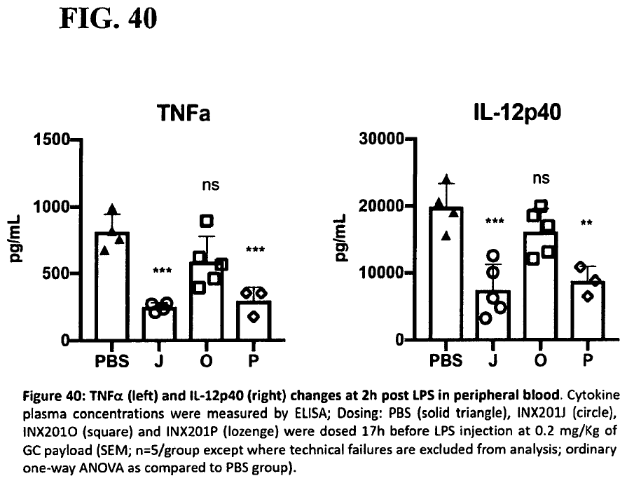

In the experiments in FIG. 40 different ADCs were dosed at 10 mg/Kg, delivering 0.2 mg/Kg of payload and their effects on particular cytokines assessed. Specifically, FIG. 40 shows TNFα (left) and IL-12p40 (right) changes at 2h post LPS in peripheral blood wherein cytokine plasma concentrations were measured by ELISA; and wherein PBS (solid triangle), INX201J (circle), INX201O (square) and INX201P (lozenge) were dosed 17h before LPS injection at 0.2 mg/Kg of GC payload (SEM; n=5/group except where technical failures are excluded from analysis; ordinary one-way ANOVA as compared to PBS group).

As shown from the data in FIG. 40, INX201P showed similar efficacy as INX201J in controlling LPS-induced cytokine response while INX201O had reduced efficacy. INX201P had comparable efficacy at 10 mg/Kg (delivering ˜0.2 mg/Kg of GC payload) to INX201J in controlling the LPS-induced TNFα and IL-12p40 responses. Comparatively, INX201O showed reduced activity.

### Experiment 7: Evaluation of In Vivo Efficacy of INX201O and INX201P Vs. INX201J in LPS-Induced Cytokine Release-Dose Response Study

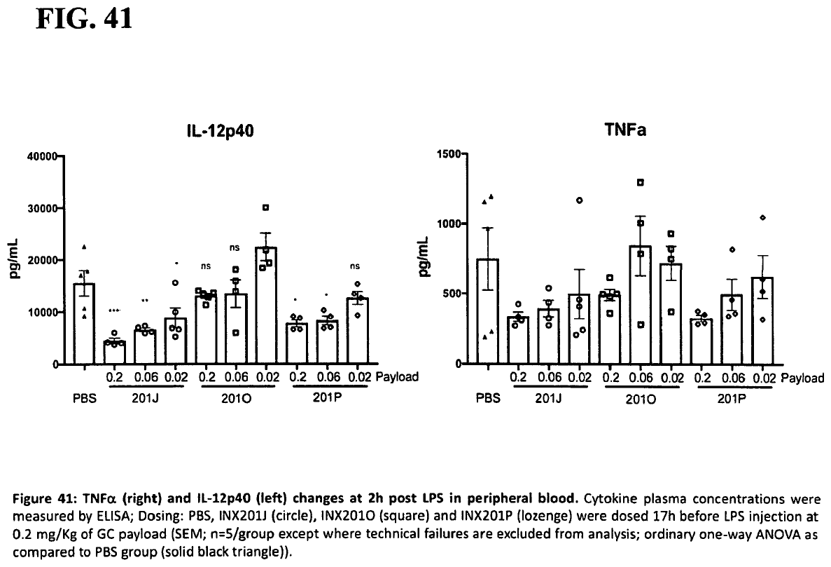

In the experiments in FIG. 41 cytokine plasma concentrations were measured by ELISA; and PBS, INX201J (circle), INX201O (square) and INX201P (lozenge) were each dosed 17h before LPS injection at 0.2 mg/Kg of GC payload (SEM; n=5/group except where technical failures are excluded from analysis; ordinary one-way ANOVA as compared to PBS group (solid black triangle)). Specifically, FIG. 41 shows TNFα (right) and IL-12p40 (left) changes at 2h post LPS in peripheral blood.

Similar to the results of the previous Experiment 6, in these experiments INX2010 showed reduced efficacy compared to INX201J. In contrast, both INX201P and INX201J showed similar potency in controlling IL-12p40 and TNFα response to LPS. Also of note, at 0.06 mg/Kg of payload, there is still potent control of the cytokine response (FIG. 41). In this repeat of a prior experiment that included dose response comparison, INX201P displayed comparable efficacy to INX201J in controlling the LPS-induced TNFα and IL-12p40 responses. INX201O again showed reduced activity.

### Experiment 8: Evaluation of In Vivo Efficacy of INX231R, INX233P Vs. INX231P in LPS-Induced Cytokine Release

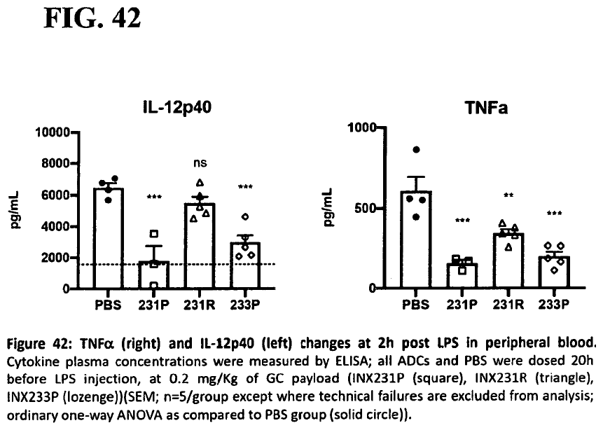

The experiment in FIG. 42 shows TNFα (right) and IL-12p40 (left) changes at 2h post LPS in peripheral blood with different ADCs. In these experiments cytokine plasma concentrations were measured by ELISA; all ADCs and PBS were dosed 20h before LPS injection, at 0.2 mg/Kg of GC payload (INX231P (square), INX231R (triangle), INX233P (lozenge)) (SEM; n=5/group except where technical failures are excluded from analysis; ordinary one-way ANOVA as compared to PBS group (solid circle)).

As can be seen from FIG. 42, INX231R (neutral dipeptide linker) showed little impact on IL-12p40 induction but significant reduction in TNFα following LPS injection. In contrast, INX233P had similar efficacy to INX231P (both with negatively charged dipeptide linkers). All ADCs were dosed at 10 mg/Kg, delivering 0.2 mg/Kg of payload. INX231R and INX233P show comparable efficacy at 10 mg/Kg (delivering ˜0.2 mg/Kg of GC payload) to INX231P in controlling the LPS-induced TNFα and IL-12p40 responses.

### Experiment 9: Evaluation of In Vivo Efficacy of INX231R, INX2010, INX231S, INX231V and INX231W Vs. INX231P in LPS-Induced Cytokine Release

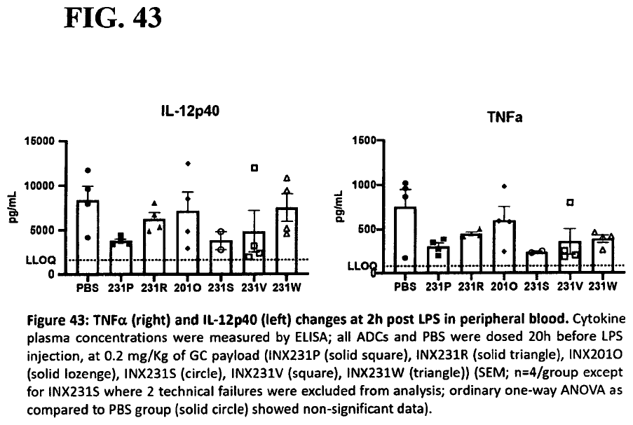

The experiment in FIG. 43 shows TNFα (right) and IL-12p40 (left) changes at 2h post LPS in peripheral blood. In these experiments cytokine plasma concentrations were measured by ELISA; all ADCs and PBS were dosed 20h before LPS injection, at 0.2 mg/Kg of GC payload (INX231P (solid square), INX231R (solid triangle), INX201O (solid lozenge), INX231S (circle), INX231V (square), INX231W (triangle)) (SEM; n=4/group except for INX231S where 2 technical failures were excluded from analysis; ordinary one-way ANOVA as compared to PBS group (solid circle) showed non-significant data). Also, in this experiment, some of the ADCs had DAR below 8 so ADC dosing was adjusted to deliver 0.2 mg/Kg of payload.

As was observed in Experiment 8, in these experiments INX231R had lower efficacy than INX231P and INX231W behaved similarly with its impact being mainly on TNFα. Additionally, INX231S and INX231V displayed similar efficacy to INX231P. Finally, as was observed in the two previous experiments, INX201O showed reduced efficacy compared to the other ADCs (FIG. 43). INX231R, INX231S, INX231V and INX231W display comparable efficacy at 10 mg/Kg (delivering ˜0.2 mg/Kg of GC payload) to INX231P in controlling the LPS-induced TNFα and IL-12p40 responses. Comparatively, INX201O showed reduced activity. Additionally, all ADCs tested except for INX201O potently delivered their GC payload as demonstrated by FKBP5 transcriptional induction with a pharmacodynamic range of at least 4 days.

### Experiment 10: Comparison of INX231R, INX2010, INX231S, INX231V and INX231W Vs. INX231P Impact on FKBP5 Transcription

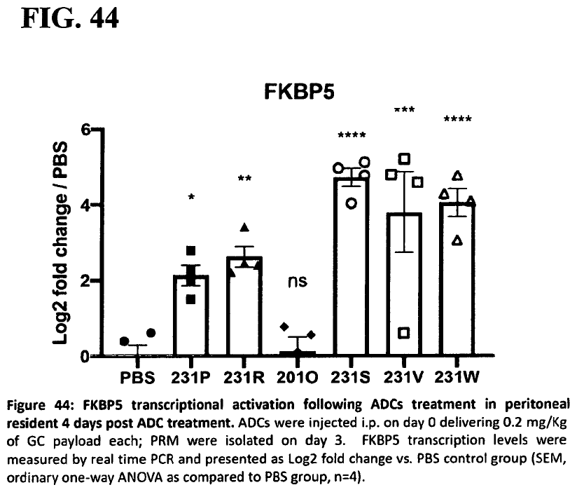

The experiment in FIG. 44 shows FKBP5 transcriptional activation following ADCs treatment in peritoneal resident 4 days post ADC treatment. In these experiments ADCs were injected i.p. on day 0 delivering 0.2 mg/Kg of GC payload each; PRMs were isolated on day 3. FKBP5 transcription levels were measured by real time PCR and presented as Log 2 fold change vs. PBS control group (SEM, ordinary one-way ANOVA as compared to PBS group, n=4).

As shown herein peritoneal resident macrophages (PRM) are exquisitely sensitive to ADCs and that GC impact on the GC target FKBP5 can be measured by real time quantitative PCR (RT-qPCR). Accordingly, PRMs were isolated on day 3 post LPS treatment (4 days post ADC dosing), RNA extracted and RT-qPCR done for FKBP5. As shown from the data in FIG. 44, except for INX2010, all ADCs induced potent FKBP5 transcription, demonstrating proper delivery of the GC payloads as well as a pharmacodynamic range of at least 4 days). INX234P, INX234A3, and INX234A4 display comparable efficacy at 10 mg/Kg (delivering ˜0.2 mg/Kg of GC payload) in controlling the LPS-induced TNFα and IL-12p40 responses. INX234T had only limited impact on TNFα. INX231R, INX231S, INX231V and INX231W display comparable efficacy at 10 mg/Kg (delivering ˜0.2 mg/Kg of GC payload) to INX231P in controlling the LPS-induced TNFα and IL-12p40 responses. Comparatively, INX201O showed reduced activity. Additionally, all ADCs tested except for INX201O potently delivered their GC payload as demonstrated by FKBP5 transcriptional induction with a pharmacodynamic range of at least 4 days.

### Experiment 11: Evaluation of In Vivo Efficacy of INX234P, INX234A3, INX234A4, INX234T and Target B—P on LPS Induced Cytokine Responses

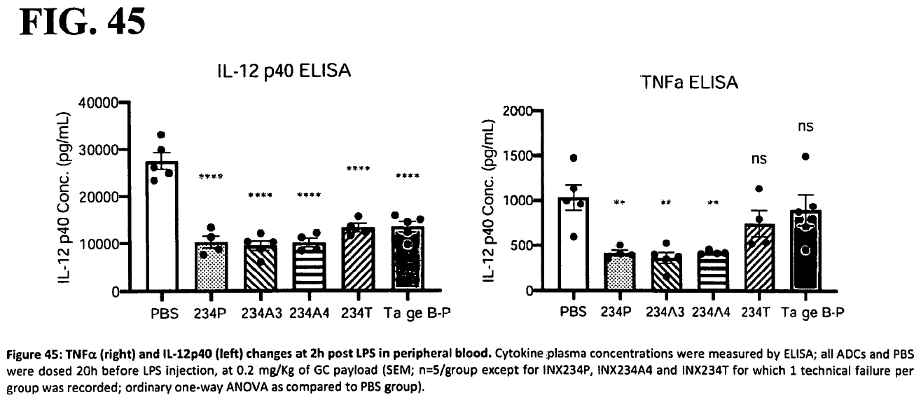

As shown in the experiment in FIG. 45, INX234P, INX234A3, and INX234A4 display comparable efficacy at 10 mg/Kg (delivering ˜0.2 mg/Kg of GC payload) in controlling the LPS-induced TNFα and IL-12p40 responses. INX234T had only limited impact on TNFα. In this experiment we also evaluated the ADC Target B—P which an antibody directed against the mouse version of a new target (target B) conjugated to INX P payload; Target B—P showed similar efficacy as INX234T with significant reduction in IL-12P40 but no changes in TNFα.

More specifically, in the FIG. 45 TNFα (right) and IL-12p40 (left) changes are shown at 2h post LPS in peripheral blood. Cytokine plasma concentrations were measured by ELISA; all ADCs and PBS were dosed 20h before LPS injection, at 0.2 mg/Kg of GC payload (SEM; n=5/group except for INX234P, INX234A4 and INX234T for which 1 technical failure per group was recorded; ordinary one-way ANOVA as compared to PBS group). The ADC which comprises an antibody to another immune cell specific antigen (other than VISTA) conjugated to INX P payload showed similar efficacy as INX234T with significant reduction in IL-12P40 but no changes in TNFα.

### Experiment 12: Evaluation of Efficacy of INX234V, INX234A5, INX234A11 vs PBS in Short Term LPS-Induced Cytokine Release

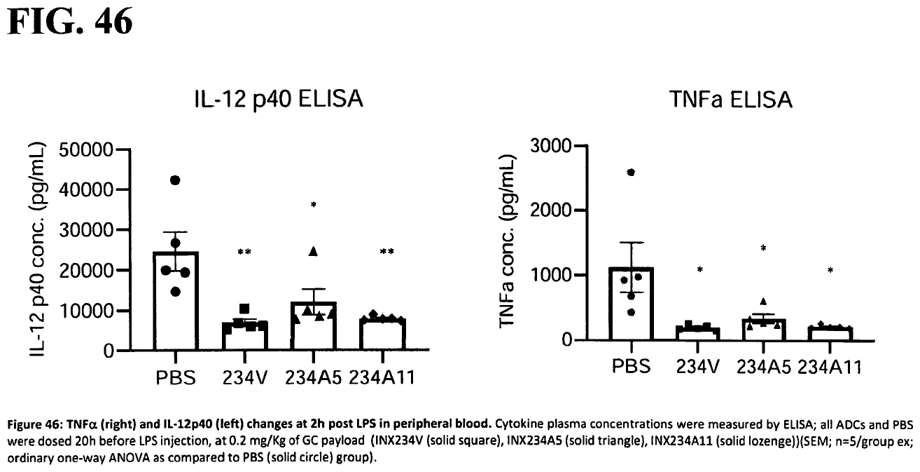

In the experiment in FIG. 46 TNFα (right) and IL-12p40 (left) changes in cytokines are shown at 2h post LPS in peripheral blood, wherein cytokine plasma concentrations were measured by ELISA; and all ADCs and PBS were dosed 20h before LPS injection, at 0.2 mg/Kg of GC payload (INX234V (solid square), INX234A5 (solid triangle), INX234A11 (solid lozenge)) (SEM; n=5/group ex; ordinary one-way ANOVA as compared to PBS (solid circle) group). The data in FIG. 46 show that INX234V, INX234A5 and INX234A11 display comparable efficacy at 10 mg/Kg (delivering ˜0.2 mg/Kg of GC payload) in controlling the LPS-induced TNFα and IL-12p40 responses.

### Experiment 13: Evaluation of Efficacy of INX231A7, INX231A12, INX231A23; INX234A1, INX234A13 vs INX234V and PS in Short Term LPS-Induced Cytokine Release

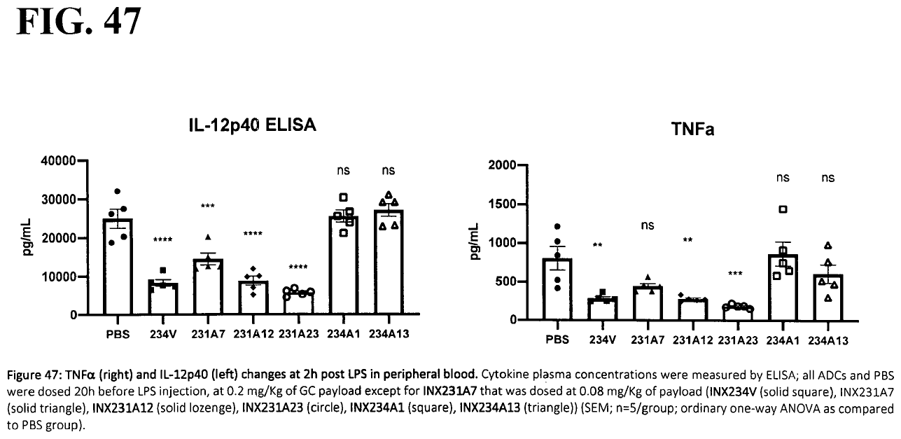

The experiments in FIG. 47 show TNFα (right) and IL-12p40 (left) changes at 2h post LPS in peripheral blood after administration of different ADCs. In these experiments cytokine plasma concentrations were measured by ELISA; all ADCs and PBS were dosed 20h before LPS injection, at 0.2 mg/Kg of GC payload (INX234V (solid square) except for INX231A7 that was dosed at 0.08 mg/Kg of payload, INX231A7 (solid triangle), INX231A12 (solid lozenge), INX231A23 (circle), INX234A1 (square), INX234A13 (triangle)) (SEM; n=5/group ex; ordinary one-way ANOVA as compared to PBS group).

As shown from the results in FIG. 47, INX234V, INX231A12 and INX231A23 showed similar potency in controlling the production of IL-12p40 and TNFα. INX2341A7 had lower potency though still driving significant changes in IL-12p40 and close to significant (P=0.052) for TNFα. (However, of note INX231A7 was dosed at 0.08 mg/Kg of payload). Also, both INX234A1 and INX234A13 had no impact on LPS-induced cytokine production. INX234V, INX231A12, INX231A23 and INX234A1 display comparable efficacy at 10 mg/Kg (delivering ˜0.2 mg/Kg of GC payload) in controlling the LPS-induced TNFα and IL-12p40 responses. INX231A7 also showed comparable efficacy while being dosed at 0.08 mg/Kg of payload. Both INX234A13 and INX234A11 showed no efficacy

### Experiment 14: Evaluation of Efficacy of INX234P, INX234A9, INX201V Vs PBS in Short Term LPS-Induced Cytokine Release

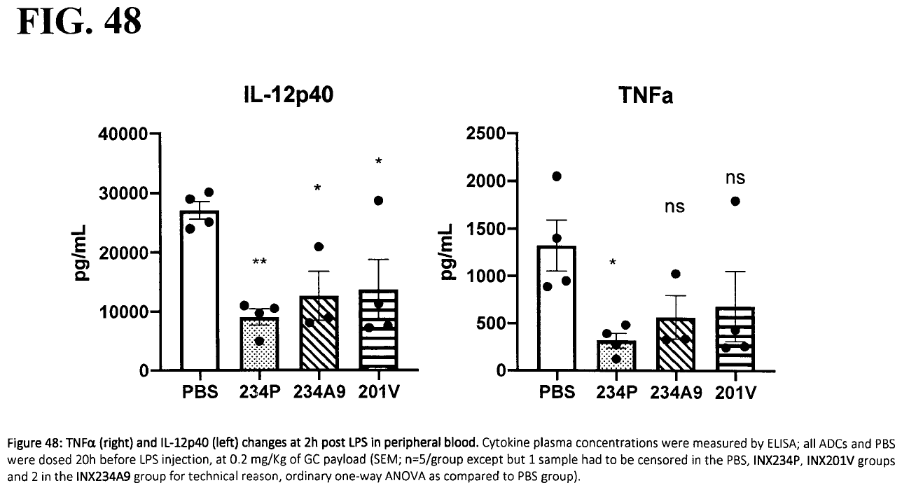

The experiments in FIG. 48 show TNFα (right) and IL-12p40 (left) changes at 2h post LPS in peripheral blood resulting from different ADCs. In these experiments cytokine plasma concentrations were measured by ELISA; all ADCs and PBS were dosed 20h before LPS injection, at 0.2 mg/Kg of GC payload (SEM; n=5/group except but 1 sample had to be censored in the PBS, INX234P, INX201V groups and 2 in the INX234A9 group for technical reason, ordinary one-way ANOVA as compared to PBS group).

As shown from the data in FIG. 48, INX234A9 and INX201V appear less potent than INX234P in preventing cytokine response; however, the diminished potency is driven by one outlier point (same animal for both cytokines showed no treatment impact which could be due to a failed injection).

### Conclusions

As disclosed herein different ADCs comprising different anti-VISTA antibodies which bind VISTA at physiologic pH and which moreover all possess short PKs and different complementarity-determining regions (CDR) and different GC payloads have been synthesized.

The data show that immune cell targeted GC delivery using each of INX201, INX231, INX234, INX240 or INX233:
efficiently decreases LPS induced cytokine responses
allows for similar potency with delivery of a ˜10-fold lower dose of GC on a mg/Kg of payload basis
may result in increased duration of pharmacodynamics of GC.

Additionally, we evaluated conjugates analogous to the INX P linker payload wherein the charge of the dipeptide linker was varied. These included positive charge (INX W), neutral charge (INX R), and negative charge (INX P). These results showed the following:
Positively charged INX W and neutral INX R were both efficacious in this model, however negatively charged INX P was more potent.
All dipeptide linker variants (positive, negative and neutral) displayed a pharmacodynamic range of at least 4 days as demonstrated by high level of expression of the GC reporter FKBP5

We further evaluated conjugates with 4 budesonide analog linker/payloads, INX N, INX O, INX P, and INX V, in addition to the initial INX J linker/payload which varied the structure of the payload. These results showed the following:
Conjugated INX N linker/payload has no potency in the short term LPS activation model, possibly reflecting a lack of efficient cleavage, or microaggregate formation of the released product of INX N.
Conjugated INX O linker/payload had an impact in this model, but showed reduced potency as compared to conjugated INX J, INX P or INX V linker/payloads.
Conjugated INXP displayed similar efficacy to conjugated INX J and INX V linker/payloads.
Conjugated INX P, INX V, and INX J linker payloads displayed a pharmacodynamic range of at least 4 days as demonstrated by high level of expression of the GC reporter gene FKBP5.
Certain payload alterations may be tolerated without disrupting potency.

We evaluated conjugates with a fluocinolone acetonide analog (INX S) in comparison to its budesonide analog counterpart (INX P). These results showed the following:
INX231P and INX231S had similar efficacy.
Both INX231P and INX231S displayed a pharmacodynamic range of at least 4 days as demonstrated by high level of expression of the GC reporter gene FKBP5.
INX234P, INX234A3 and INX234A4 have similar potency while INX234T showed more limited (and non-significant) control of LPS induced TNFα production.
INX234V, INX234A5 and INX234A11 showed similar potency in controlling LPS induced IL-12p40 and TNFα production.
INX231A7, INX231A12 and INX231A23 showed similar potency as INX234V potency in controlling LPS induced IL-12p40 and TNFα production while INX234A1 and INX234A 13 had no impact.
INX234V, INX231A12, INX231A23 and INX234A1 display comparable efficacy at 10 mg/Kg (delivering ˜0.2 mg/Kg of GC payload) in controlling the LPS-induced TNFα and IL-12p40 responses. INX231A7 also showed comparable efficacy while being dosed at 0.08 mg/Kg of payload. By contrast INX234A13 and INX234A11 showed no efficacy.

Moreover, and importantly, we showed that an exemplary payload according to the invention, the INX P payload, conserved its potency when delivered to immune cells when conjugated to an antibody that binds to a distinct target (not VISTA) which comprises a murine ortholog of an antigen referred to as target B. The results indicate that the INX P payload, when conjugated to an antibody that binds to another immune cell antigen also was efficiently internalized by myeloid cells, similar to the results seen when this same payload was conjugated to an anti-VISTA antibody. Based thereon we anticipate that payloads according to the invention may be conjugated to other antibodies which bind to other (non-VISTA) immune cell antigens, e.g., other antigens expressed by one or more types of immune cells including by way of example B, T, NK, myeloid, macrophage, Treg, neutrophil, and dendritic cells, among other immune cell types and will also effectively deliver payloads according to the invention into these immune cells.

Also, we demonstrated that two conjugates with linker payloads containing a spirocenter near the point of cleavage, INX234A9 and INX201V appear less potent than INX234P in controlling LPS induced IL-12p40 and TNFα production though the diminished potency is driven by a possible outlier data point (same animal for both cytokines showed no treatment impact, therefore these potency differences could be attributable to a failed injection).

### REFERENCES

1—Vermeer et al. (2003) Glucocorticoid-induced increase in lymphocytic FKBP51 messenger ribonucleic acid expression: a potential marker for glucocorticoid sensitivity, potency, and bioavailability. J Clin Endocrinol Metab. January; 88 (1): 277-84
2—McPherson M J, et al. (2017) Glucocorticoid receptor agonist and immunoconjugates thereof, U.S. Ser. No. 15/611,037

### Example 8: Anti-VISTA Antibody Drug Conjugates have Limited Impact on Non-VISTA Expressing Cells

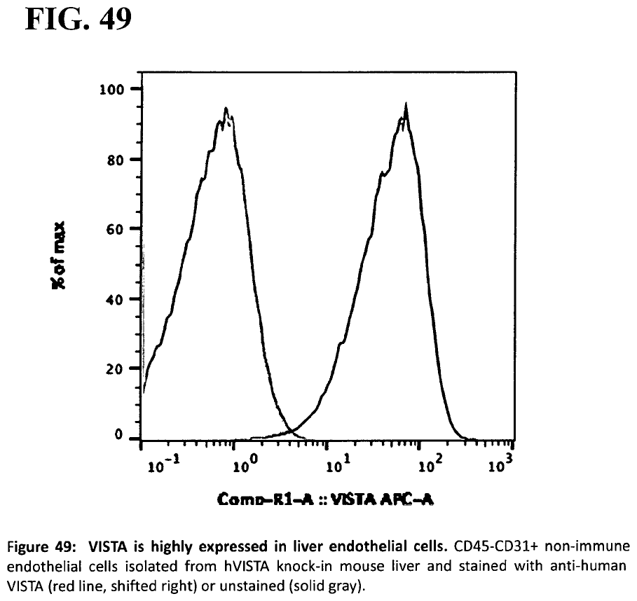

The impact of exemplary ADCs on non-target (non-VISTA) expressing cells was assessed in the experiments described in in this example and shown in the experiment in FIG. 49. The objective of these studies was to validate the targeting specificity of the inventive ADCs to VISTA expressing cells/tissues as compared to free dexamethasone (Dex). To monitor/confirm GC delivery and activity, we measured by quantitative Real Time PCR (qRT-PCR) the transcriptional activation of FKBP5, a sensitive and early GC response gene (Vermeer et al., (2003) Glucocorticoid-induced increase in lymphocytic FKBP51 messenger ribonucleic acid expression: a potential marker for glucocorticoid sensitivity, potency, and bioavailability. J Clin Endocrinol Metab. January; 88 (1): 277-84). In these experiments which are disclosed in detail below INX201J or free Dex was delivered in vivo via intraperitoneal (i.p.) injection, followed by isolation of VISTA expressing splenocytes and non-VISTA expressing cells from liver, brain and adrenal gland. RNA was then extracted and FKBP5 transcriptional levels evaluated.

### Materials and Methods

### Methods

Dex was injected i.p. at 2h before mouse euthanasia and cell isolation, which corresponds to peak FKBP5 induction. INX201J was injected 20h before mouse euthanasia and cell isolation, to provide sufficient time for ADC processing and peak FKBP5 induction. The control group injected with PBS only was included to define FKBP5 transcript baseline.

### Test Agents and Dosage

### Antibodies

INX201 (Aragen, Lot #BP-3200-019-6) is a humanized anti-human VISTA antibody on a human IgG1/kappa backbone with L234A/L235A/E269R/K322A silencing mutations in the Fc region.
INX201J (Abzena, Lot #s JZ-0556-025-1, JZ-0556-027, JZ-0556-013) is the INX201 antibody with a drug/antibody ratio (DAR) of 8.0, conjugated via full modification of the interchain disulfides. The linker/payload (J) is based on a previously reported linker/payload (U.S. Ser. No. 15/611,037). It consists of a negatively charged protease sensitive linker with a budesonide analog payload (INX J-2).

These antibodies were diluted in PBS and injected intraperitoneal (i.p.) in a volume of 0.2 ml to deliver a specified dose.

### Dexamethasone

Dexamethasone sterile injection from Phoenix, NDC 57319-519-05, was diluted in PBS and dosed as described via i.p. injection.

### Mice

The hVISTA KI mice were bred on site (Center for Comparative Medicine and Research at Dartmouth). All the experiments were done in female mice enrolled between 9 and 15 weeks of age.

### Cell Isolation

After euthanasia, spleen, liver, adrenal gland and brain were dissected and dissociated mechanically. After passage on a 40 μm filter, cell pellets were resuspended in the RNA lysis buffer (See below).

### RNA Preparation and Real Time PCR

Cell pellets from different tissues were resuspended in 0.4 ml RNeasy lysis buffer from RNeasy Plus Mini kit (Qiagen, PN: 74136) and homogenized with a 20G needle for 5 times. RNA was isolated following manufacturer's instructions and eluted in in 30 or 40 μl H₂O (RNase/DNase free). RNA concentration was assessed on Nanodrop.

Reverse transcription was done using Taqman reverse transcription reagents (#N8080234) and following manufacturer's instructions. Quantitative Real-Time PCR was done using Taqman master mix 2× kit (#4369016) and Taqman primers for mouse FKBP5 (Mm00487401_m1), and mouse HPRT as housekeeping gene (Mm446968_m1) and run on a QuantStudio3 from Applied Biosystem.

Ct data were converted to ΔCt and ΔΔCt or Log 2 fold changes to PBS.

### Results

Briefly the Liver dissociation kit and Liver Endothelial Cell Isolation kit from Miltenyi were used (130-105-807 and 130-092-007 respectively) to isolate liver endothelial cells from hVISTA KI mice. As shown in the experiment in FIG. 49, CD45 negative (non-immune) CD31 positive (endothelial) cells exhibit high level of VISTA expression (red line VISTA; solid grey no antibody). Specifically, FIG. 49 shows that VISTA is highly expressed in liver endothelial cells, particularly CD45⁻CD31⁺ non-immune endothelial cells isolated from hVISTA knock-in mouse liver and stained with anti-human VISTA (red line, shifted right) or unstained (solid gray).

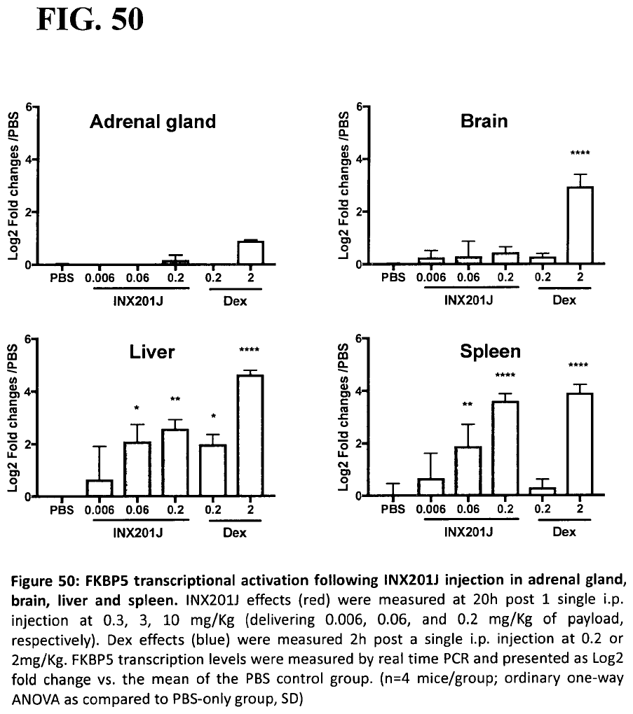

In the experiments shown in FIG. 50 we evaluated the impact of INX201J vs. Dex on non-VISTA expressing tissues (adrenal gland, brain, and liver) and VISTA expressing spleen in female hVISTA KI mice. Specifically, as shown in FIG. 50 FKBP5 transcriptional activation following INX201J injection in adrenal gland, brain, liver and spleen. INX201J effects were measured at 20h post 1 single i.p. injection at 0.3, 3, 10 mg/Kg (delivering 0.006, 0.06, and 0.2 mg/Kg of payload, respectively). Dex effects were measured 2h post a single i.p. injection at 0.2 or 2 mg/Kg. FKBP5 transcription levels were measured by real time PCR and presented as Log 2 fold change vs. the mean of the PBS control group. (n=4 mice/group; ordinary one-way ANOVA as compared to PBS-only group).

As can be seen from the results in FIG. 50, no signal above baseline was detected in adrenal gland and brain following INX201J injection while Dex at 2 mg/Kg led to a slight increase in FKBP5 transcript for adrenal gland and a robust increase in brain. In the liver, FKBP5 was detected at similar levels with INX201J or Dex at 0.2 mg/Kg of payload, and elevated levels with Dex at 2 mg/Kg.

Further, a clear dose dependent induction with INX201J was observed in spleen with a 10-fold increase in FKBP5 signal with INX201J at 0.2 mg/Kg of payload when compared to Dex at 0.2 mg/Kg of payload. By contrast a comparable response with Dex was only achieved at 2 mg/Kg.

### Conclusions

The data shows that INX201J at 3 and 10 mg/Kg (0.06 and 0.2 mg/Kg of payload) induces FKBP5 expression in VISTA-expressing splenocytes, but not adrenal gland or brain (FIG. 49). In the liver, INX201J when dosed at 3 and 10 mg/Kg (0.06 and 0.2 mg/Kg of payload) modestly induces FKBP5, likely due to this tissue's abundance of immune cells and robust VISTA expression in liver endothelial cells (FIG. 50). By contrast, Dex at the therapeutic dose of 2 mg/Kg induced FKBP5 induction in splenocytes, and this same dose induced robust levels of FKBP5 in brain and liver, and modest levels of FKPB5 in the adrenal gland.

### Example 9: In Vitro Potency of Inventive Steroid Payloads and Antibody Glucocorticoid Conjugates in Human Peripheral Blood Mononuclear Cells

In this example we assessed the in vitro potency of different steroid payloads in human peripheral blood mononuclear cells steroids in an in vitro model of inflammation. The presence of LPS results in PBMCS proliferation and cytokine release (Jansky, L., Reymanova, P., & Kopecky, J. (2003), “Dynamics of cytokine production in human peripheral blood mononuclear cells stimulated by LPS, or infected by Borrelia”, Physiological Research, 52 (5), 593-5981).

### Materials and Methods

### Methods

The potency of novel steroids was assessed in a model of LPS stimulated human peripheral blood mononuclear cells (PBMCS). Stimulated PBMCs in this assay produce multiple pro-inflammatory cytokines1. Steroid potency was judged in these studies by the ability to reduce the expression of stimulation-related cytokines in a dose dependent manner relative to no treatment at 24 hrs.

The objective of the present studies was to evaluate the potency of novel glucocorticoids generated at ImmuNext, identified as INX-SM-GC, in a well characterized in vitro model of inflammation, either as the free small molecules, or conjugated to an antibody through a peptide linker. Human PBMCS when stimulated with LPS produce several pro-inflammatory cytokines and this cytokine response can be dramatically inhibited by glucocorticoids (GC). We used budesonide, a very potent and clinically relevant GC as a comparator in our studies.

### Materials and Methods

### Experiment Design

In all the following experiments, human PBMC, isolated from 1-2 healthy donors per study, were stimulated with LPS to induce cytokine production.

Cells were treated with serially diluted GCs or GC conjugates (1000-0.2 nM payload) to identify the dose dependent potency of the individual drugs, with budesonide as a positive control. LPS was added either immediately after addition of free payload, or in the case of conjugates, four hours after addition of the conjugate.

In our preliminary experiments, we identified IL-6 and IL-1b as highly GC responsive cytokines. Therefore, after incubating PBMC with GC or antibody conjugated GCs for 24h, cell supernatants were collected and assessed for IL-6 and IL-1b cytokine levels via ELISA.

### Reagents

### Test Payloads

Budesonide: 10 mM in DMSO
INX-SM-1 (Abzena): 5 mM in DMSO
INX-SM-2 (Abzena): 2 mM in DMSO
INX-SM-3 (O2H): 10 mM in DMSO
INX-SM-53 (O2H): 10 mM in DMSO (S stereoisomer of INX-SM-3)
INX-SM-4 (O2H): 20 mM in DMSO
INX-SM-54 (O2H): 10 mM in DMSO (S stereoisomer of INX-SM-4)
INX-SM-6 (O2H): 10 mM in DMSO
INX-SM-56 (O2H): 10 mM in DMSO (S stereoisomer of INX-SM-6)
INX-SM-7 (O2H): 2 mM in DMSO
INX-SM-9 (O2H): 2 mM in DMSO
INX-SM-10 (O2H): 2 mM in DMSO.
INX-SM-13 (O2H): 2 mM in DMSO
INX-SM-24 (O2H): 2 mM in DMSO
INX-SM-31 (O2H): 2 mM in DMSO
INX-SM-32 (O2H): 2 mM in DMSO
INX-SM-33 (O2H): 2 mM in DMSO
INX-SM-35 (O2H): 2 mM in DMSO
INX-SM-74 (O2H): 2 mM in DMSO (S stereoisomer of INX-SM-24)
INX-SM-43 (O2H): 20 mM in DMSO
INX-SM-44 (O2H): 20 mM in DMSO
INX-SM-45 (O2H): 20 mM in DMSO
INX-SM-46 (O2H): 20 mM in DMSO
INX-SM-36 (O2H): 20 mM in DMSO
INX-SM-37 (O2H): 20 mM in DMSO
INX-SM-J2 (O2H): 20 mM in DMSO
INX-SM-14 (O2H): 20 mM in DMSO.
INX-SM-15 (O2H): 20 mM in DMSO.
INX-SM-17 (O2H): 20 mM in DMSO
INX-SM-34 (O2H): 20 mM in DMSO
INX-SM-40 (O2H): 20 mM in DMSO
INX-SM-47 (O2H): 20 mM in DMSO
INX-SM-49 (O2H): 20 mM in DMSO
INX-SM-18 (O2H): 20 mM in DMSO
INX-SM-32 (O2H): 20 mM in DMSO
INX-SM-48 (O2H): 20 mM in DMSO
INX-J2 (INX-SM-J2) (O2H): 20 mM in DMSO
Dexamethasone (Sigma cat #D4902): 20 mM in DMSO

### Test Conjugates

INX231J (Abzena, lot #JZ-0556-013-1) is INX231 conjugated with a drug/antibody ratio (DAR) of 8.0, conjugated via full modification of the interchain disulfides. The linker/payload (INX J) is based on a published linker/payload (U.S. Ser. No. 15/611,037). It consists of a negatively charged protease sensitive linker with a budesonide analog payload (INX J2).
INX231P (Abzena, lot #JZ-0556-017-1) is INX231 with a DAR of 8.0, conjugated via full modification of the interchain disulfides. The linker/payload (INX P) consists of a negatively charged protease sensitive linker with a budesonide analog payload (INX-SM-3).
INX231V (Abzena, lot #PP-0920-014-2) is INX231 with a DAR of 7.8, conjugated via modification of the interchain disulfides. The linker/payload (INX V) consists of a negatively charged protease sensitive linker with a budesonide analog payload (INX-SM-32).
INX234P (Abzena, lot #JZ-0556-017-2) is INX234 with a DAR of 8.0, conjugated via full modification of the interchain disulfides. The linker/payload (INX P) consists of a negatively charged protease sensitive linker with a budesonide analog payload (INX-SM-3).
INX234A4 (Abzena, lot #PP-0924-023-2) is the INX234 antibody with a DAR of 7.9, conjugated via modification of the interchain disulfides. The linker/payload (INX A4) consists of a negatively charged protease sensitive linker with a budesonide analog payload (INX-SM-43).
INX231S (Abzena, lot #PP-0920-014-1) is INX231 with a DAR of 6.9, conjugated via modification of the interchain disulfides. The linker/payload (INX S) consists of a negatively charged protease sensitive linker with a fluocinolone acetonide analog payload (INX-SM-24).
INX234T (Abzena, lot #PP-0924-023-3) is the INX234 antibody with a DAR of 8.0, conjugated via full modification of the interchain disulfides. The linker/payload (INX T) consists of a negatively charged protease sensitive linker with a budesonide analog payload (INX-SM-3) that is phosphorylated.
INX234A3 (Abzena, lot #PP-0924-023-1) is the INX234 antibody with a DAR of 8.0, conjugated via full modification of the interchain disulfides. The linker/payload (INX A3) consists of a positively charged protease sensitive linker with a budesonide analog payload (INX-SM-32).
INX201J (Abzena, lot #s: JZ-0556-025-1, JZ-0556-027, JZ-0556-013) is the INX201 antibody conjugated to linker/payload via full modification of the interchain disulfides with a DAR of 8.0. The linker/payload (INX J) is based on a published linker/payload (U.S. Ser. No. 15/611,037). It consists of a negatively charged protease sensitive linker with a budesonide analog payload (INX J2).
INX201L (Abzena, lot #JZ-0556-026-1) is the INX201 antibody with a DAR of 8.0, conjugated via full modification of the interchain disulfides. The linker/payload (INX L) is based on a published linker/payload (U.S. Ser. No. 15/611,037). It consists of a negatively charged protease sensitive linker with a budesonide analog payload (INX J-2) that is phosphorylated.
INX234A11 (Abzena, lot #RJS-1054-001) is the INX234 antibody with DAR of 8.0, conjugated via full modification of the interchain disulfides. The linker/payload (INX A11) consists of a negatively charged protease sensitive linker (Asn/gly) with a budesonide analog payload (INX-SM-32).
INX234V (Abzena, lot #RJS-1054-003) is the INX234 antibody with a DAR of 8.0, conjugated via full modification of the interchain disulfides. The linker/payload (INX V) consists of a negatively charged protease sensitive linker with a budesonide analog payload (INX-SM-32).
INX234A5 (Abzena, lot #RJS-1054-002) is the INX234 antibody with a DAR of 7.9, conjugated via modification of the interchain disulfides. The linker/payload (INX A5) consists of a negatively charged protease sensitive linker with a budesonide analog payload (INX-SM-44).
INX231A23 (Abzena, lot #RJS-1054-006-001) is the INX231 antibody with a DAR of 7.34, conjugated via modification of the interchain disulfides. The linker/payload (INX A23) consists of a negatively charged protease sensitive linker with a fluocinolone acetonide analog payload (INX-SM-25).
INX231A12 (Abzena, lot #RJS-1054-007-002) is the INX231 antibody with a DAR of 6.99, conjugated via modification of the interchain disulfides. The linker/payload (INX A12) consists of a negatively charged protease sensitive linker with a fluocinolone acetonide analog payload (INX-SM-25) that is phosphorylated.
INX231A7 (Abzena, lot #RJS-1054-007-001) is the INX231 antibody with a DAR of 7.8, conjugated via modification of the interchain disulfides. The linker/payload (INX A7) consists of a negatively charged protease sensitive linker with a budesonide analog payload (INX-SM-32) that is phosphorylated.
INX234A13 (Abzena, lot #RJS-1054-007-003) is the INX234 antibody with a DAR of 5.82, conjugated via modification of the interchain disulfides. The linker/payload (INX A13) consists of a negatively charged protease sensitive linker with a budesonide analog payload (INX-SM-34).
INX234A1 (Abzena, lot #RJS-1054-004) is the INX234 antibody with a DAR of 7.6, conjugated via modification of the interchain disulfides. The linker/payload (INX A1) consists of a negatively charged protease sensitive linker with a budesonide analog payload (INX-SM-35).
INX1302 (ATUM, lot #78597.2.a) is an Fc silent anti-human Target B antibody
INX1302P (Abzena, Lot #PP-0924-019-002) is an Fc silent anti-human Target B antibody, INX1302, with a DAR of 8.0, conjugated via full modification of the interchain disulfides. The linker/payload (INX P) consists of a negatively charged protease sensitive linker with a budesonide analog payload (INX-SM-3).
INX1400P (Abzena, lot #PP-0924-019-001) is an Fc silent anti-human Target A antibody with a DAR of 8.0, conjugated via full modification of the interchain disulfides. The linker/payload (INX P) consists of a negatively charged protease sensitive linker with a budesonide analog payload (INX-SM-3).
INX234J (Abzena, lot #JZ-0556-013-2) is the INX234 antibody with a DAR of 8.0, conjugated via full modification of the interchain disulfides. The linker/payload (INX J) is based on a published linker/payload (U.S. Ser. No. 15/611,037). It consists of a negatively charged protease sensitive linker with a budesonide analog payload (INX J2).
INX234A9 (Abzena, lot #AF-1114-007-1) is the INX234 antibody with a DAR of 7.3, conjugated via modification of the interchain disulfides. The linker/payload (INX A9) consists of a negatively charged protease sensitive linker with a budesonide analog payload (INX-SM-46).
INX201V (Abzena, lot #SCG-1120-012-1) is the INX201 antibody with a DAR of 7.86, conjugated via modification of the interchain disulfides. The linker/payload (INX V) consists of a negatively charged protease sensitive linker with a budesonide analog payload (INX-SM-32).
INX231V (Pearl River, lot #101-1_02) is the INX231 antibody with a DAR of 3.66, conjugated via modification of the interchain disulfides. The linker/payload (INX V) consists of a negatively charged protease sensitive linker with a budesonide analog payload (INX-SM-32).
INX201P (Pearl River, lot #101-2_02) is the INX201 antibody with a DAR of 4.52, conjugated via modification of the interchain disulfides. The linker/payload (INX P) consists of a negatively charged protease sensitive linker with a budesonide analog payload (INX-SM-3).
INX201A23 (Pearl River, lot #101-3_02) is the INX201 antibody with a DAR of 4.34, conjugated via modification of the interchain disulfides. The linker/payload (INX A23) consists of a negatively charged protease sensitive linker with a fluocinolone acetonide analog payload (INX-SM-25).
INX201A23 (Pearl River, lot #101-4_01) is the INX201 antibody with a DAR of 7.71, conjugated via modification of the interchain disulfides. The linker/payload (INX A23) consists of a negatively charged protease sensitive linker with a fluocinolone acetonide analog payload (INX-SM-25).
INX201V (Abzena, lot #SCG-1120-032-1) is the INX201 antibody with a DAR of 3.95, conjugated via modification of the interchain disulfides. The linker/payload (INX V) consists of a negatively charged protease sensitive linker with a budesonide analog payload (INX-SM-32).

### Cell Culture Media

RPMI 1640 without L-glutamine (VWR cat #16750-084)
Penicillin/Streptomycin/Glutamine (ThermoFisher cat #10378016)
1M Hepes (Gibco cat #15630-080)
Human AB serum (Valley Biomedical cat #HP1022HI)

### Other Reagents

Lipopolysaccharides from Escherichia coli 0111: B4 (Sigma cat #L2630).
Ficoll-Paque Plus (GE Healthcare cat #17-1440-03)

### ELISA Kits

Human IL-6 ELISA MAX Deluxe (Biolegend cat #430504)
Human IL-1β ELISA MAX Deluxe (Biolegend cat #437004)

### PBMCS Preparation

Human PBMCs were isolated under sterile conditions from apheresis cones obtained from the Blood Donor Program at the Dartmouth Hitchcock Medical Center from deidentified healthy human donors.

The blood was transferred to a 50 ml Falcon tube and diluted with PBS to 30 ml. 13 ml of Ficoll-Paque Plus (Sigma Aldrich) was slowly layered under the blood, and tubes were centrifuged at 850×g for 20 min at RT with mild acceleration and no brake.

Mononuclear cells were collected from the plasma/Ficoll interface, resuspended in 50 ml of PBS and centrifuged at 300×g for 5 min. Cells were resuspended in PBS and counted.

### Assay Protocol

Isolated PBMCs were resuspended in RPMI 1640 containing 10% human A/B serum, 10 mM Hepes, 1x Penicillin/Streptomycin/L-Glutamine (assay media).

Cells were plated in flat bottom 96 well plates at a final concentration of 150,000 cells/well, with technical duplicates for each condition.

Test agents were serially diluted in the assay media and added to a final concentration of 1,000 nM-1 nM or 0.2 nM depending on the assay or as a no treatment control.

LPS stimulation was added to a final concentration of 1 ng/ml. For delayed LPS studies, LPS was added 4 hrs after addition of antibody drug conjugate (INX231J, INX231P, INX231V).

Cells were placed at 37° C. in a 5% CO2 incubator for 24h before supernatant harvesting.

Human IL-1β and IL-6 ELISA kits were used on supernatants according to vendor protocols.
All graphs were prepared with GraphPad (Prism).

### Results

### Experiment 1: Assessment of Inhibitory Effects of Steroid Payloads INX-SM-3, INX-SM-53, INX-SM-4, INX-SM-54, and INX-SM-1 on Cytokine Production of LPS Stimulated Human PBMCs

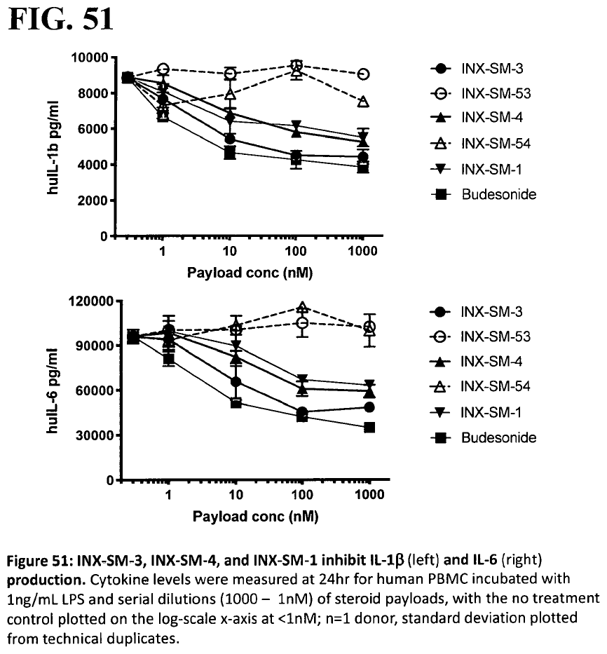

In the experiment shown in FIG. 51, we evaluated the anti-inflammatory potency of the novel INX-GC payloads INX-SM-3, INX-SM-4, INX-SM-1, INX-SM-53 and INX-SM-54. PBMCS from one donor were tested. As shown in FIG. 51, INX-SM-3, INX-SM-4, and INX-SM-1 inhibit both IL-1β and IL-6 production, INX-SM-3 appearing the most potent compound among these three. By contrast, the S stereoisomers at the acetal position—INX-SM-53 and INX-SM-54—did not show inhibition.

From the data in the FIG. 51 it can be seen that INX-SM-3, INX-SM-4, and INX-SM-1 inhibit IL-13 (left) and IL-6 (right) production. Cytokine levels were measured at 24 hr for human PBMCS incubated with 1 ng/ml LPS and serial dilutions (1000-1 nM) of steroid payloads, with the no treatment control plotted on the log-scale x-axis at <1 nM; n=1 donor, standard deviation plotted from technical duplicates.

### Experiment 2: Assess Inhibitory Effects of Steroid Payloads INX-SM-6, and INX-SM-56 and Confirm Effects of INX-SM-1, INX-SM-3 and INX-SM-4 on Cytokine Production of LPS Stimulated Human PBMCs

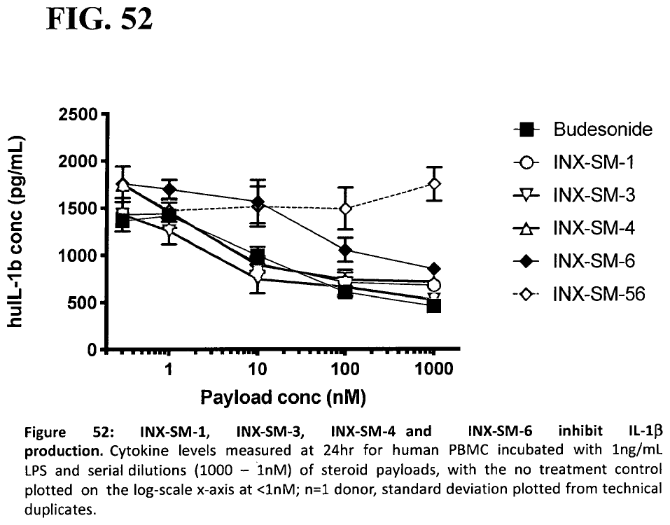

In the experiment in FIG. 52, we confirmed the anti-inflammatory potency of novel glucocorticoid payloads INX-SM-3, INX-SM-4, INX-SM-1 and assessed the potency of an additional compounds INX-SM-6 and INX-SM-56. PBMCS from one donor were tested. Particularly, in FIG. 52 cytokine levels were measured at 24 hr for human PBMCS incubated with 1 ng/mL LPS and serial dilutions (1000-1 nM) of steroid payloads, with the no treatment control plotted on the log-scale x-axis at <1 nM; n=1 donor, standard deviation plotted from technical duplicates.

As shown in FIG. 52, INX-SM-1, INX-SM-3, INX-SM-4 and INX-SM-6 show inhibition in IL-1β production. INX-SM-3 appears to be again the most potent compound among these compounds tested. By contrast, the S stereoisomer at the acetal position-INX-SM-56-did not show inhibition.

### Experiment 3: Assessment of Inhibitory Effects of Steroid Payloads INX-SM-9, INX-SM-31, and INX-SM-35 on Cytokine Production of LPS Stimulated Human PBMC

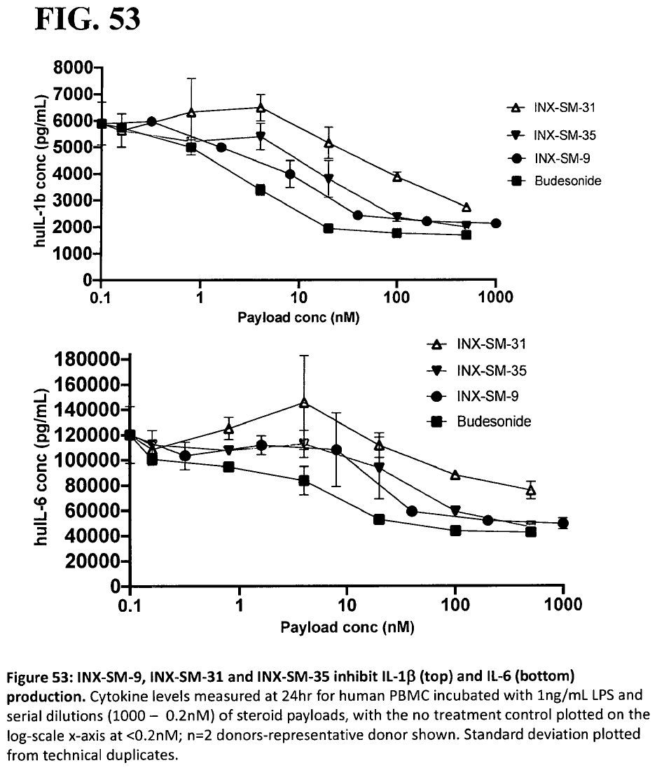

In this experiment, we evaluated the anti-inflammatory potency of novel glucocorticoid payloads INX-SM-9, INX-SM-31 and INX-SM-35. PBMCS from two donors were tested. Specifically, FIG. 53 shows INX-SM-9, INX-SM-31 and INX-SM-35 inhibit IL-1β (top) and IL-6 (bottom) production. Cytokine levels measured at 24 hr for human PBMCS incubated with 1 ng/mL LPS and serial dilutions (1000-0.2 nM) of steroid payloads, with the no treatment control plotted on the log-scale x-axis at <0.2 nM; n=2 donors-representative donor shown. Standard deviation plotted from technical duplicates.

As shown from the results in FIG. 53, INX-SM-9, INX-SM-35 and INX-SM-31 show dose dependent inhibition in IL-1β production. It appears INX-SM-31 is the least potent compound among these compounds tested.

### Experiment 4: Assessment of Inhibitory Effects of Exemplary Steroid Payload INX-SM-32 on Cytokine Production of LPS Stimulated Human PBMC

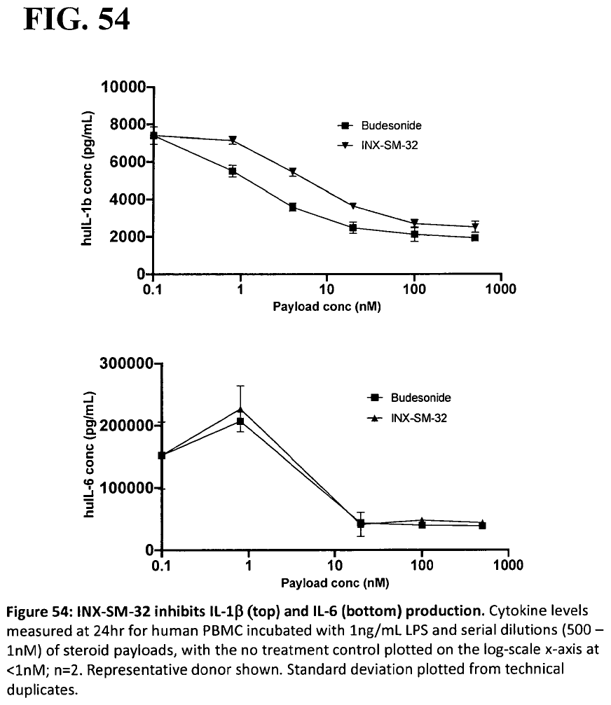

In this experiment, we evaluated the anti-inflammatory potency of novel glucocorticoid payload INX-SM-32. The experiment was repeated with PBMCS from a second donor. Specifically the data in the FIG. 54 shows that INX-SM-32 inhibits IL-1β (top) and IL-6 (bottom) production. Cytokine levels measured at 24 hr for human PBMCS incubated with 1 ng/mL LPS and serial dilutions (500-1 nM) of steroid payloads, with the no treatment control plotted on the log-scale x-axis at <1 nM; n=2. Representative donor shown. Standard deviation plotted from technical duplicates.

As shown from the data in FIG. 54, INX-SM-32 shows dose dependent inhibition in IL-1β and IL-6 production.

### Experiment 5: Assessment of Inhibitory Effects of Exemplary Steroid Payloads INX-SM-10 and INX-SM-33 on Cytokine Production of LPS Stimulated Human PBMCs

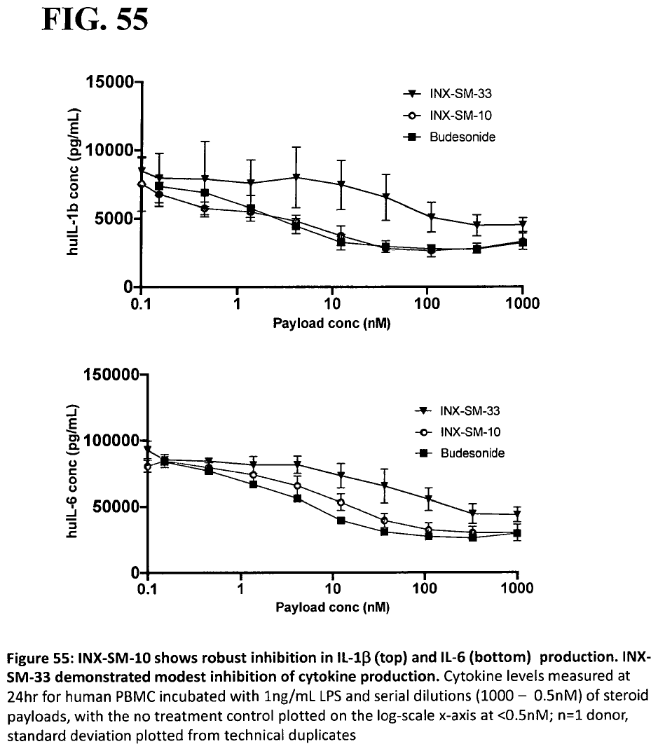

In this experiment, we evaluated the anti-inflammatory potency of novel glucocorticoid payloads INX-SM-10, and INX-SM-33. PBMCS from one donor were tested. FIG. 55 shows that INX-SM-10 elicits robust inhibition in IL-1β (top) and IL-6 (bottom) production. INX-SM-33 demonstrated modest inhibition of cytokine production. Cytokine levels measured at 24 hr for human PBMCS incubated with 1 ng/mL LPS and serial dilutions (1000-0.5 nM) of steroid payloads, with the no treatment control plotted on the log-scale x-axis at <0.5 nM; n=1 donor, standard deviation plotted from technical duplicates.

More specifically, the data in FIG. 55, show that INX-SM-10, and INX-SM-33 elicit dose dependent inhibition in IL-1β production with INX-SM-33 appearing to be the least potent compound among these compounds tested.

### Experiment 6: Assessment of Inhibitory Effects of Exemplary Steroid Payloads INX-SM-2, INX-SM-7, INX-SM-13, INX-SM-24, and INX-SM-74 on Cytokine Production of LPS Stimulated Human PBMCs

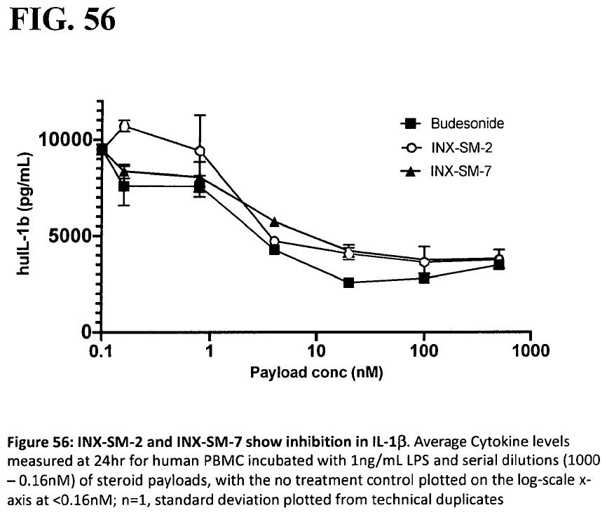

In this experiment in FIG. 56, we evaluated the anti-inflammatory potency of novel glucocorticoid payloads INX-SM-2 and INX-SM-7. Additionally, INX-SM-13 (halogenation at C9), INX-SM-24 (halogenation at C6 and C9) and INX-SM-74 (S stereoisomer of INX-SM-24) vs INX-SM-3 (no-halogenation) were assessed to establish the impact of halogenation of the steroid ring for these compounds. This experiment used PBMCS from single donor.

Specifically, the data in FIG. 56 show dose-dependent inhibition in IL-1β production by INX-SM-2 and INX-SM-7. Specifically, in the Figure shows average cytokine levels measured at 24 hr for human PBMCS incubated with 1 ng/mL LPS and serial dilutions (1000-0.16 nM) of steroid payloads, with the no treatment control plotted on the log-scale x-axis at <0.16 nM; n=1, standard deviation plotted from technical duplicates.

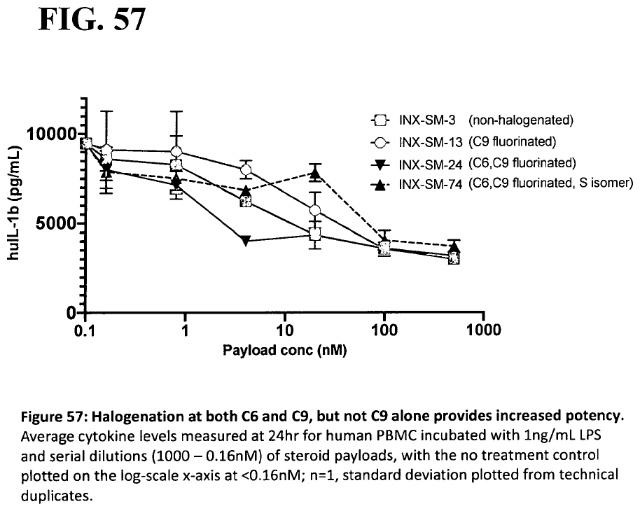

Also, as shown in FIG. 57, an assessment of the impact of halogenation on the potency of INX-SM-3 demonstrated that fluorination at both the C6 and C9 positions of INX-SM-24 leads to increased potency over the non-fluorinated INX-SM-3. However, fluorination at the C9 position alone (INX-SM-13) did not lead to increased potency over the non-fluorinated payload (INX-SM-3). Of note, the S stereo isomer of INX-SM-24 also showed dose dependent potency. This is unlike several non-fluorinated S stereo isomers we tested which did not show potency in similar in vitro studies (INX-SM-53; INX-SM-54, and INX-SM-56).

The data in FIG. 57 show that halogenation at both C6 and C9, but not C9 alone provides increased potency. Average cytokine levels measured at 24 hr for human PBMCs incubated with 1 ng/ml LPS and serial dilutions (1000-0.16 nM) of steroid payloads, with the no treatment control plotted on the log-scale x-axis at <0.16 nM; n=1, standard deviation plotted from technical duplicates.

### Experiment 7: Assessment of Anti-Inflammatory Effects of Different ADCs Including Those Conjugated to Antibodies Specific to Other Immune Cell Targets which Comprise Exemplary Inventive Linker Payload (INX P Linker Payload)

In this experiment we assessed the anti-inflammatory effects of ADCs which comprised antibodies specific to other immune cell targets (2 distinct immune cell antigens other than VISTA) which ADCs comprise an exemplary inventive linker payload (INX P linker payload) Particularly, the antibodies are directed against TARGET B (INX1400P) and TARGET A (INX1302P), and the effects thereof on LPS stimulated human PBMCs were detected. As noted above, Target A and Target B are distinct antigens from each other, and from VISTA and are both highly expressed by specific immune cell types.

In this study we evaluated the anti-inflammatory effects of antibodies conjugated with the INX P linker payload, but directed against other cell surface targets, anti-Target B (INX1400P) and anti-Target A (INX1302P). Unconjugated anti-Target A antibody (INX1302) was added as a control for potential antibody effect. PBMC from one donor were tested.

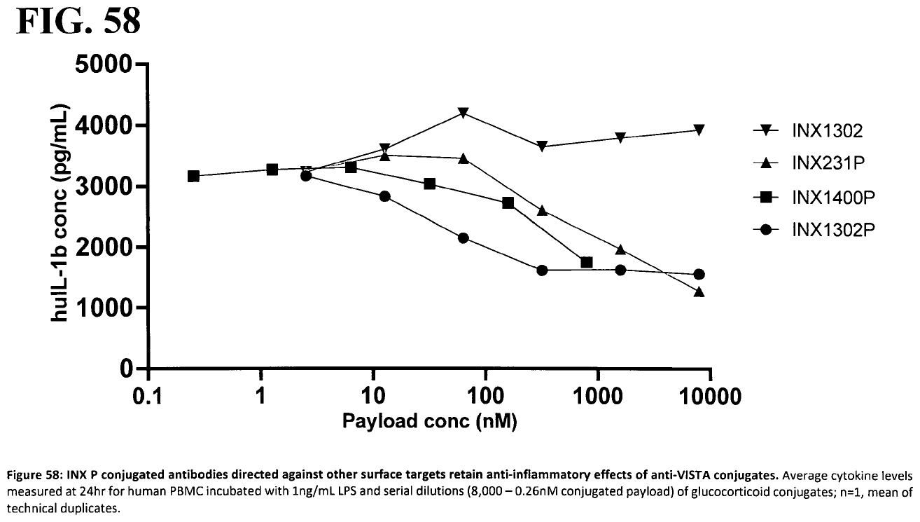

As shown in FIG. 58, all three INXP conjugates were able to deliver active free payload and retain the potency observed with anti-VISTA INX P conjugates. FIG. 58 shows that INX P conjugated antibodies directed against TARGET B and TARGET A (2 different immune cell specific antigens other than VISTA) also elicit potent anti-inflammatory effects. Average cytokine levels measured at 24 hr for human PBMC incubated with 1 ng/mL LPS and serial dilutions (8,000-0.26 nM conjugated payload) of glucocorticoid conjugates; n=1, mean of technical duplicates.

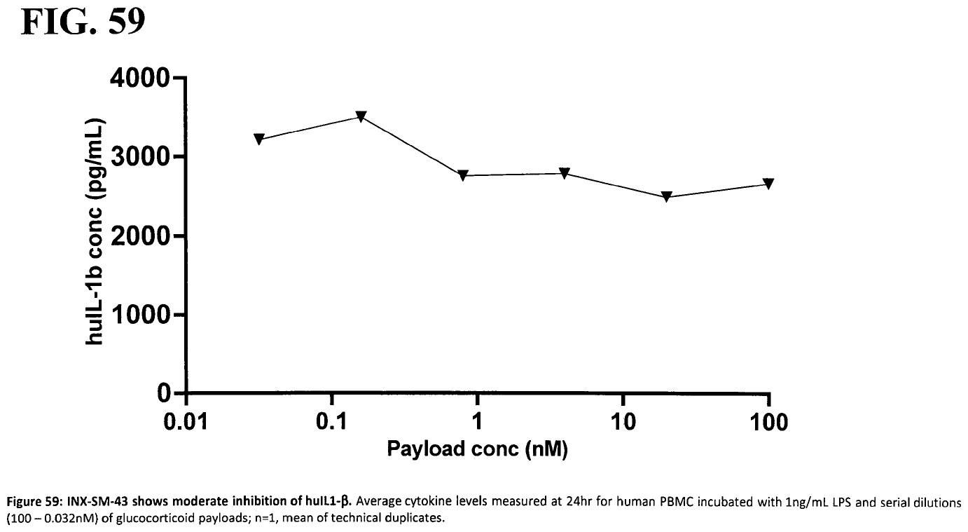

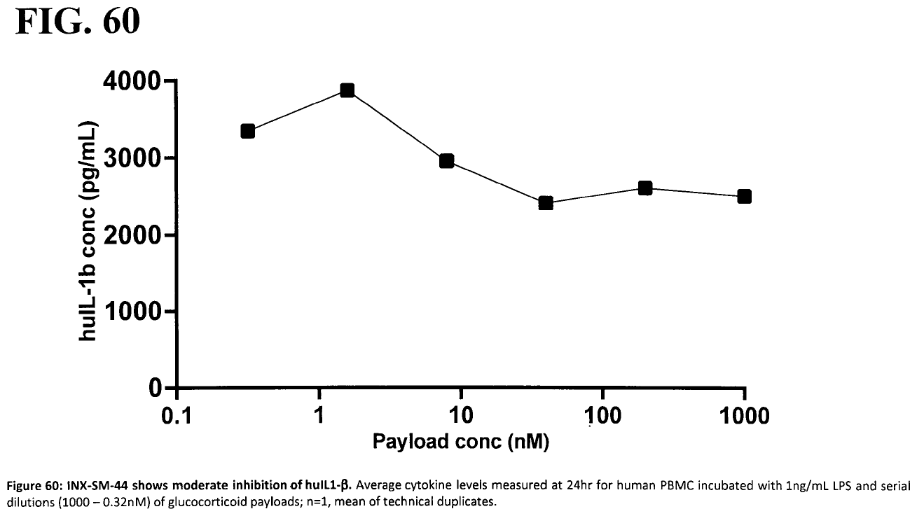

Additionally, two free payloads, INX-SM-43 and INX-SM-44 were assessed for anti-inflammatory effect. Both payloads were able to have some measure of anti-inflammatory effect as shown in FIG. 59 (INX-SM-43) and FIG. 60 (INX-SM-44)

Specifically the experiment in FIG. 59 shows that INX-SM-43 elicits moderate inhibition of huIL1-β. Average cytokine levels measured at 24 hr for human PBMC incubated with 1 ng/mL LPS and serial dilutions (100-0.032 nM) of glucocorticoid payloads; n=1, mean of technical duplicates. The experiment in FIG. 60 shows also that INX-SM-44 elicits moderate inhibition of huIL1-β. Average cytokine levels measured at 24 hr for human PBMC incubated with 1 ng/ml LPS and serial dilutions (1000-0.32 nM) of glucocorticoid payloads; n=1, mean of technical duplicates.

### Experiment 8: Assessment of Anti-Inflammatory Potency of Exemplary Glucocorticoid Payloads INX-SM-25, INX-SM-45 and INX-SM-46 vs INX-SM-3)

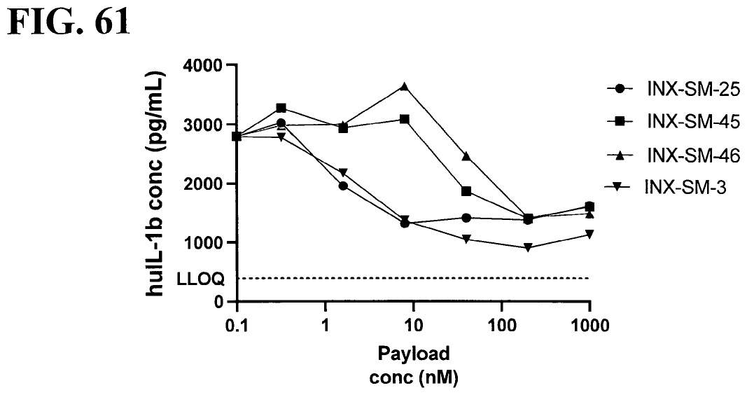

In this study, we evaluated the anti-inflammatory potency of novel glucocorticoid payloads INX-SM-25, INX-SM-45 and INX-SM-46 vs INX-SM-3. PBMC from one donor were tested. Specifically, FIG. 61 shows INX-SM-25 and INX-SM-3 show robust inhibition in IL-1B production. INX-SM-45 and INX-SM-46 demonstrated more modest inhibition of cytokine production. Cytokine levels measured at 24 hr for human PBMC incubated with 1 ng/mL LPS and serial dilutions (1000-0.5 nM) of steroid payloads, with the no treatment control plotted on the log-scale x-axis at <0.5 nM; n=1 donor, mean of technical duplicates plotted.

As shown in the experiment in FIG. 61, all free payloads tested show dose dependent inhibition in IL-1β production and that INX-SM-45 and INX-SM-46 apparently have reduced potency relative to INX-SM-25 and INX-SM-3.

### Experiment 9: Assessment of Anti-Inflammatory Potency of Novel Glucocorticoid Payloads in Full Conjugated Form (INX231J, INX231P, INX231V)

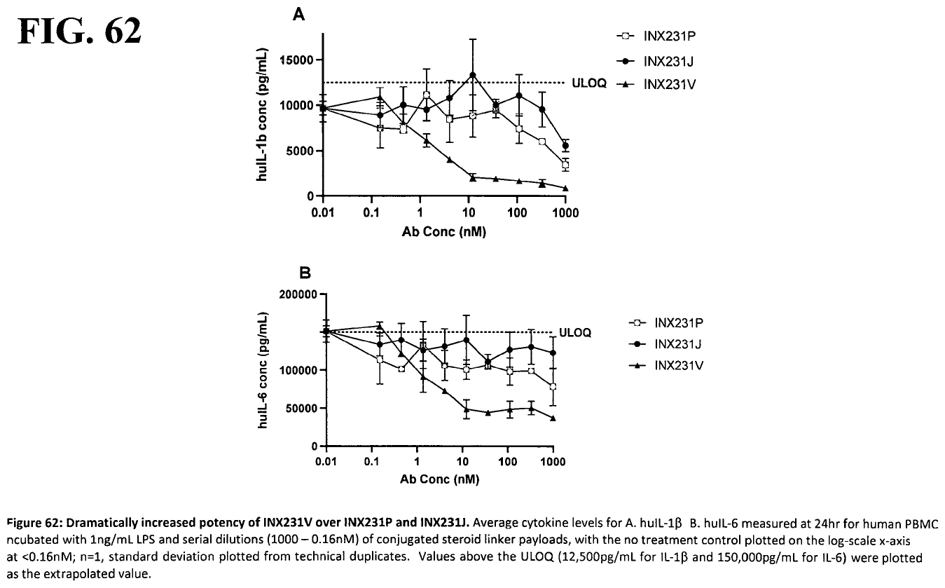

In this study the data of which is in FIG. 62, we evaluated the anti-inflammatory potency of novel glucocorticoid payloads in full conjugated form. INX J (INX-J2) a previously reported linker/payload was compared to novel linker payloads INX P containing the INX-SM-3 payload and INX V containing the INX-SM-32 payload. All linker payloads were conjugated to INX231, an anti-VISTA antibody, as INX231J, INX231P and INX231V with an approximate DAR of 8. Of note, all payloads (INX-SM-3), (INX-SM-32) and (INX-SM-3 and INX-J2) had similar potency relative to budesonide in in vitro stimulated PBMC assays.

Conjugates were added to PBMC of a single donor as a dose response of payload concentrations and allowed to incubate for 4 hrs. This delay allowed time for payload processing. After 4 hrs, LPS was added, and samples were incubated for 24 hrs before assessment of IL-1b and IL-6. As shown in FIG. 62, INX231V had dramatically enhanced potency over INX231P and INX231J. INX231P had some enhancement of potency over INX231J (the literature comparator). This finding is unexpected as the same linker was used (gly/glu) and all free payloads had similar potency relative to budesonide.

This finding is unexpected as the same linker was used (gly/glu) and all free payloads had similar potency relative to budesonide. Particularly, FIG. 62 shows dramatically increased potency of INX231V over INX231P and INX231J. Average cytokine levels for A. hulL-1β B. hull-6 measured at 24 hr for human PBMC incubated with 1 ng/ml LPS and serial dilutions (1000-0.16 nM) of conjugated steroid linker payloads, with the no treatment control plotted on the log-scale x-axis at <0.16 nM; n=1, standard deviation plotted from technical duplicates. Values above the ULOQ (12,500 pg/mL for IL-1b and 150,000 pg/mL for IL-6) were plotted as the extrapolated value.

### Experiment 10: Assessment of Anti-Inflammatory Potency of Novel Glucocorticoid Payloads in Full Conjugated Form (INX231J, INX231P, INX234P, INX231V, INX234A4, INX231S, INX234T, INX234A3, INX201J, INX201L, INX234A11, INX234V, INX234A5)

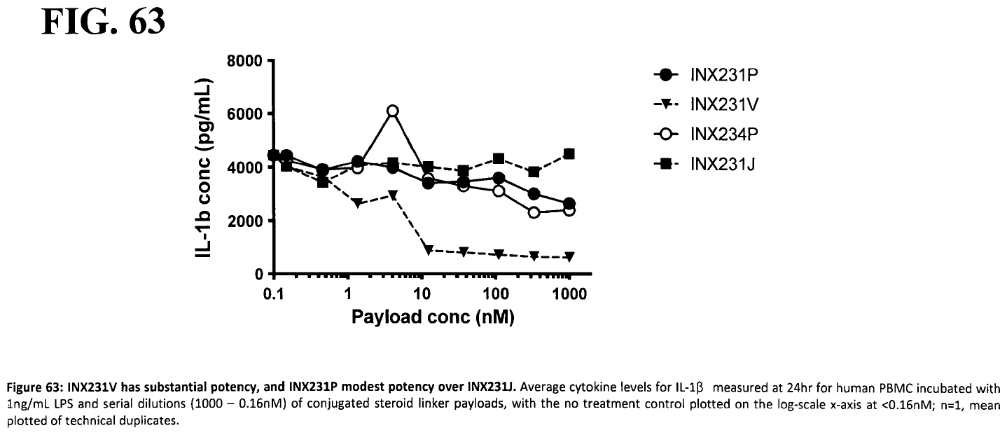

In this study the data of which is in FIG. 63, we evaluated the anti-inflammatory potency of novel glucocorticoid payloads in full conjugated form. Specifically, INX231 conjugated with INX J (INX-J2), a previously reported linker/payload, was compared to INX231P, INX234P, INX231V, INX234A4, INX231S, INX234T, INX234A3, INX201J, INX201L, INX234A11, INX234V, INX234A5. PBMCs from one donor was tested.

As shown in the experiment in FIG. 63, V when conjugated to identical anti-target antibodies, conjugated INX V has a substantial potency enhancement over conjugated INX P and INX J. Conjugated INX P has a modest enhancement over conjugated INX J. This is in line with accumulation data in human PBMC disclosed herein which show 235x released payload of INX231V relative to INX231P and >3x released payload for INX231P over INX231J. Of note, INX234P and INX231P have similar potency, reflecting the similar properties of these antibodies.

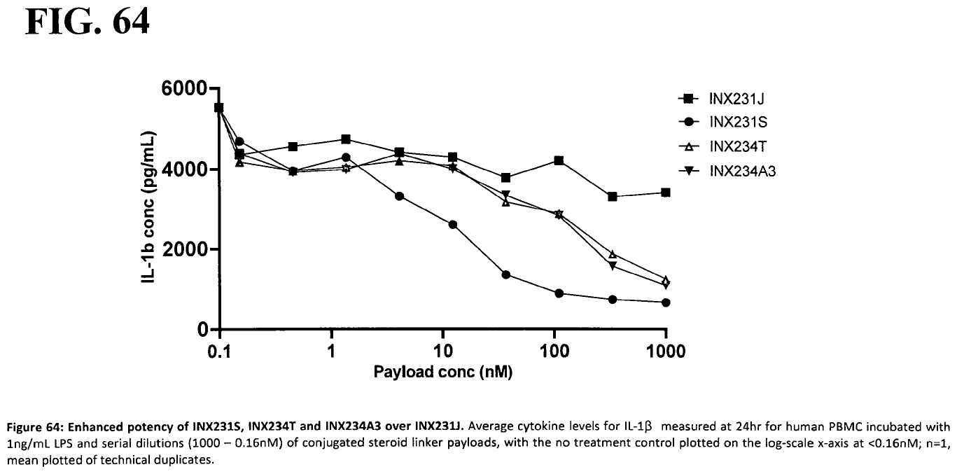

The experiment in FIG. 64 further shows that INX231V has substantial potency, and INX231P modest potency over INX231J. Average cytokine levels for IL-1β measured at 24 hr for human PBMC incubated with 1 ng/ml LPS and serial dilutions (1000-0.16 nM) of conjugated steroid linker payloads, with the no treatment control plotted on the log-scale x-axis at <0.16 nM; n=1, mean plotted of technical duplicates. Specifically, as shown in the experiment in FIG. 64, INX231S has substantial potency over other conjugates likely due to the enhanced potency of its released C6/C9 fluorinated payload (INX-SM-24). INX234T (phosphorylated version of INX P) and INX234A3 (positively charged linker variant of INX V) also have greater potency than INX231J. Specifically, the experiment in FIG. 64 shows the enhanced potency of INX231S, INX234T and INX234A3 over INX231J. Average cytokine levels for IL-1β measured at 24 hr for human PBMC incubated with 1 ng/mL LPS and serial dilutions (1000-0.16 nM) of conjugated steroid linker payloads, with the no treatment control plotted on the log-scale x-axis at <0.16 nM; n=1, mean plotted of technical duplicates.

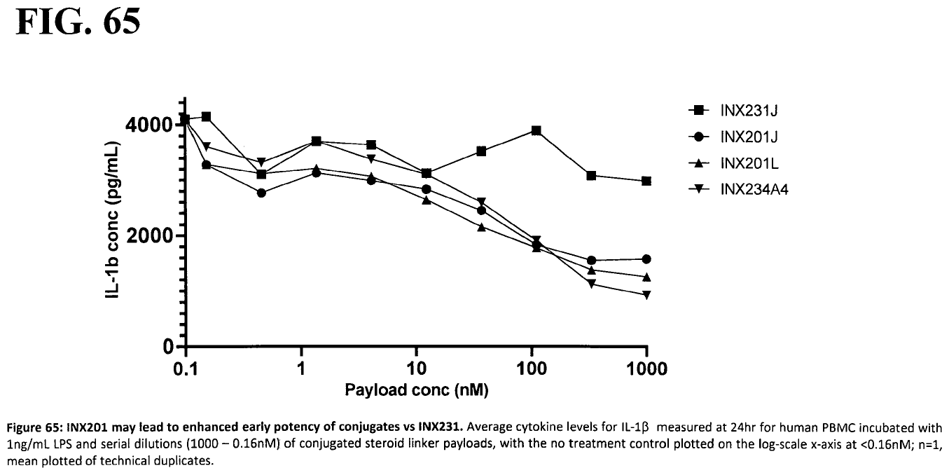

Though we have observed INX231 and INX234 conjugates to behave similarly, linker payloads conjugated to INX201 may have enhanced potency in vitro due to the more rapid internalization of the INX201 antibody. In fact, as shown in the experiment in FIG. 65, INX201J and IN201L have enhanced potency relative to the INX231J conjugate. Of note, INX234A4 (likely comparable to a INX231A4 conjugated form) has enhanced potency over INX231J. Particularly, FIG. 65 shows that INX201 may lead to enhanced early potency of conjugates vs INX231. Average cytokine levels for IL-1β measured at 24 hr for human PBMC incubated with 1 ng/mL LPS and serial dilutions (1000-0.16 nM) of conjugated steroid linker payloads, with the no treatment control plotted on the log-scale x-axis at <0.16 nM; n=1, mean plotted of technical duplicates.

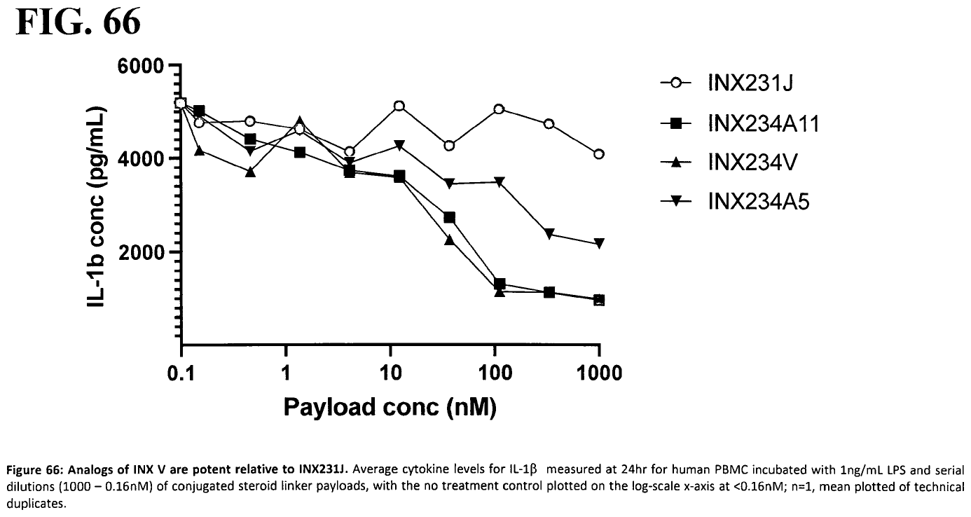

Moreover, as shown in the experiment in FIG. 66, a negatively charged linker variant of V, INX A11, which utilizes an Asn/gly linker in contrast to INX V's Glu/gly linker has a similar potency enhancement over INX J. Of note, INX234A5 also has a spiro[2.2]heptane, and shows enhanced potency over INX231J suggestive of its importance in release and subsequent potency. Specifically, the experiment in FIG. 66 shows that different analogs of INX V are potent relative to INX231J. Average cytokine levels for IL-1β measured at 24 hr for human PBMC incubated with 1 ng/mL LPS and serial dilutions (1000-0.16 nM) of conjugated steroid linker payloads, with the no treatment control plotted on the log-scale x-axis at <0.16 nM; n=1, mean plotted of technical duplicates.

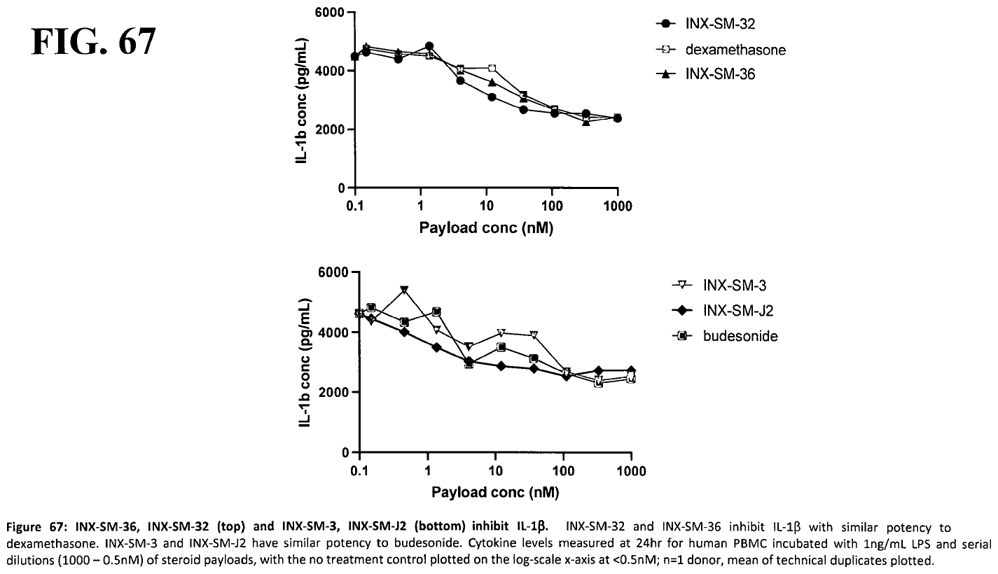

We further compared the efficacy of INX-SM-3, INX-J2, budesonide, dexamethasone, INX-SM-32, INX-SM-36. As shown in the experiment in FIG. 67: INX-SM-36, INX-SM-32 (top) and INX-SM-3, INX-SM-J2 (bottom) inhibit IL-1β. INX-SM-32 and INX-SM-36 inhibit IL-1B with similar potency to dexamethasone. INX-SM-3 and INX-J2 have similar potency to budesonide. Cytokine levels measured at 24 hr for human PBMC incubated with 1 ng/ml LPS and serial dilutions (1000-0.5 nM) of steroid payloads, with the no treatment control plotted on the log-scale x-axis at <0.5 nM; n=1 donor, mean of technical duplicates plotted.

### Experiment 11: Assessment of Anti-Inflammatory Potency of Novel Glucocorticoid Payloads (INX-SM-3, INX-SM-37 and INX-SM-32) to a Reported Glucocorticoid Payload (INX-J2)

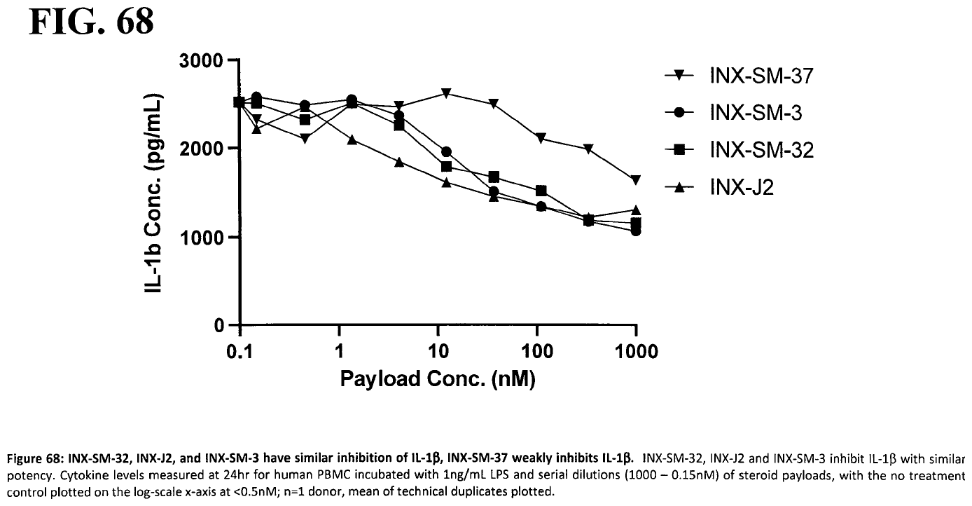

In this study we evaluated the anti-inflammatory potency of the free glucocorticoid INX-J2 (a known free glucocorticoid) relative to our novel glucocorticoids, INX-SM-3, INX-SM-37 and INX-SM-32. As shown in the experiment in FIG. 68, in a direct comparison of the free payloads of INX J, INX P, and INX V linker payloads (INX-J2, INX-SM-3, and INX-SM-32 respectively), all show similar potency. Also, the results suggest that INX-J2 may show a modest potency enhancement over INX-SM-32 and INX-SM-3. This similarity in potency conclusively demonstrates that the potency enhancement observed with INX V and INX P conjugates (and related analogs) over INX J conjugates as shown in FIG. 68, and in the experiment in FIG. 64, is not due to enhanced potency of the released payload, but is likely due to enhanced accumulation. By contrast, INX-SM-37 has weak potency relative to the other free payloads.

The data in FIG. 68 show that INX-SM-32, INX-J2, and INX-SM-3 have similar inhibition of IL-1β, that INX-SM-37 weakly inhibits IL-1β, and that INX-SM-32, INX-J2 and INX-SM-3 inhibit IL-1β with similar potency. Cytokine levels measured at 24 hr for human PBMC incubated with 1 ng/mL LPS and serial dilutions (1000-0.15 nM) of steroid payloads, with the no treatment control plotted on the log-scale x-axis at <0.5 nM; n=1 donor, mean of technical duplicates plotted.

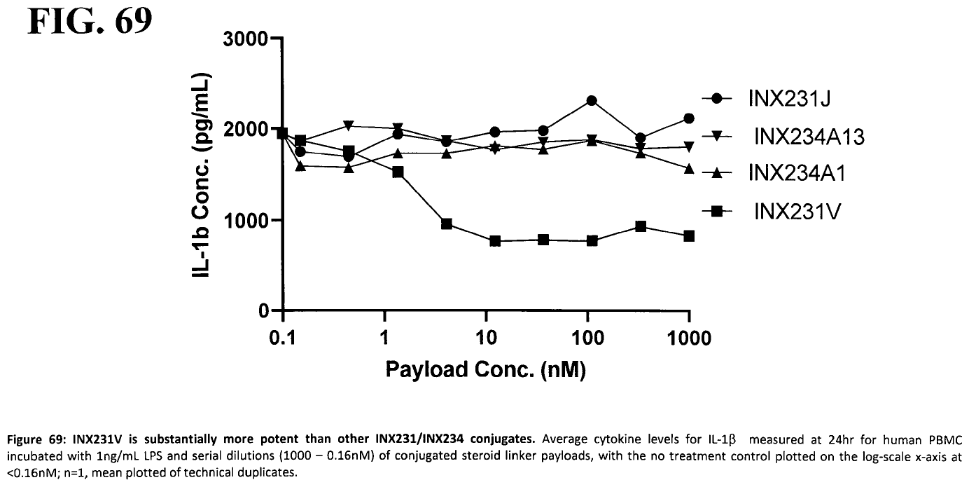

The experiment in FIG. 69 further shows that INX231V is substantially more potent than other INX231/INX234 conjugates. In this experiment the average cytokine levels for IL-1β measured at 24 hr for human PBMC incubated with 1 ng/mL LPS and serial dilutions (1000-0.16 nM) of conjugated steroid linker payloads, with the no treatment control plotted on the log-scale x-axis at <0.16 nM; n=1, mean plotted of technical duplicates.

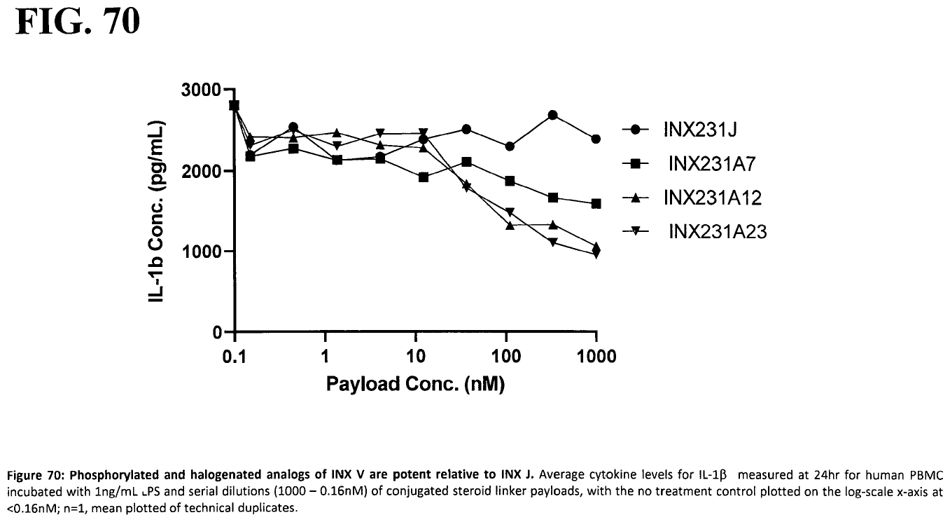

As shown in FIG. 70, phosphorylated (INX231A7, INX231A12) and halogenated forms (INX231A23 and INX231A12) of INX V also demonstrate enhanced potency over INX 231J. Particularly the experiment in FIG. 70 shows that phosphorylated and halogenated analogs of INX V are potent relative to INX J. The average cytokine levels for IL-1β measured at 24 hr for human PBMC incubated with 1 ng/mL LPS and serial dilutions (1000-0.16 nM) of conjugated steroid linker payloads, with the no treatment control plotted on the log-scale x-axis at <0.16 nM; n=1, mean plotted of technical duplicates.

### Experiment 12: Assessment of Anti-Inflammatory Potency of Novel Glucocorticoid Payloads (INX-SM-14, INX-SM-15, INX-SM-17, INX-SM-40, INX-SM-34, INX-SM-47, INX-SM-49 and INX-J2)

In this study, we evaluated the anti-inflammatory potency of novel glucocorticoid payloads. INX-J2, a previously reported payload, was compared to payloads INX-SM-14, INX-SM-15, INX-SM-17, INX-SM-40, INX-SM-34, INX-SM-47 and INX-SM-49. PBMCs from one donor were tested.

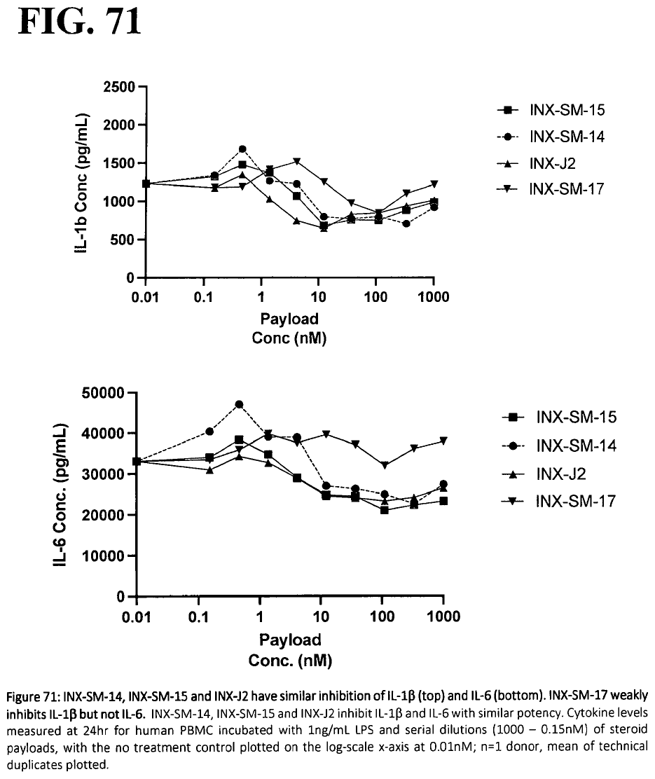

As shown in FIG. 71, INX-SM-14 and INX-SM-15 have similar ability to inhibit IL-1β and IL-6 as INX-J2. As shown in FIGS. 71, 72, and 73, other GC payloads, INX-SM-17, INX-SM-34, INX-SM-40, INX-SM-47, and INX-SM-49 weakly inhibit IL-1β and IL-6 or only weakly inhibit IL-1β (INX-SM-17) relative to INX-J2.

Particularly, the data in FIG. 71 show that INX-SM-14, INX-SM-15 and INX-J2 have similar inhibition of IL-1β (top) and IL-6 (bottom). INX-SM-17 weakly inhibits IL-1β but not IL-6. INX-SM-14, INX-SM-15 and INX-J2 inhibit IL-1β and IL-6 with similar potency. Cytokine levels measured at 24 hr for human PBMC incubated with 1 ng/ml LPS and serial dilutions (1000-0.15 nM) of steroid payloads, with the no treatment control plotted on the log-scale x-axis at 0.01 nM; n=1 donor, mean of technical duplicates plotted.

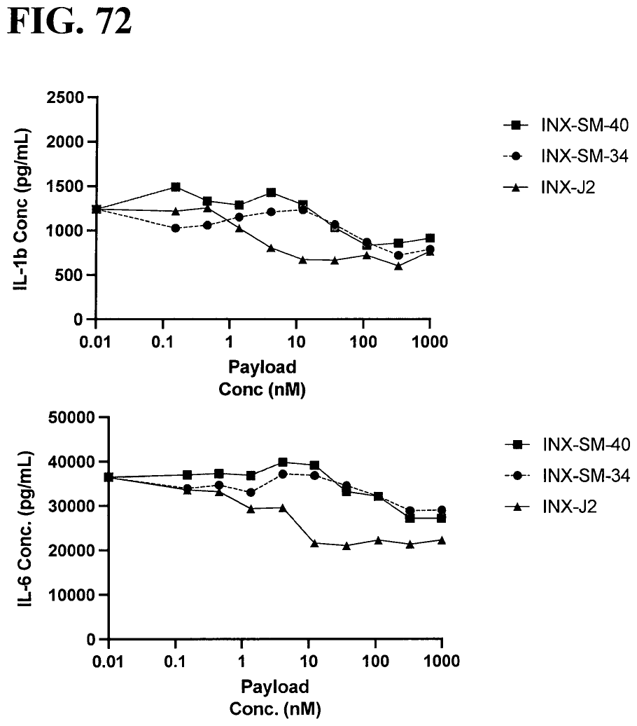

Further, the data in FIG. 72 show that INX-SM-40 and INX-SM-34 weakly inhibit IL-1β (top) and IL-6 (bottom) relative to INX-J2. Cytokine levels measured at 24 hr for human PBMC incubated with 1 ng/mL LPS and serial dilutions (1000-0.15 nM) of steroid payloads, with the no treatment control plotted on the log-scale x-axis at 0.01 nM; n=1 donor, mean of technical duplicates plotted.

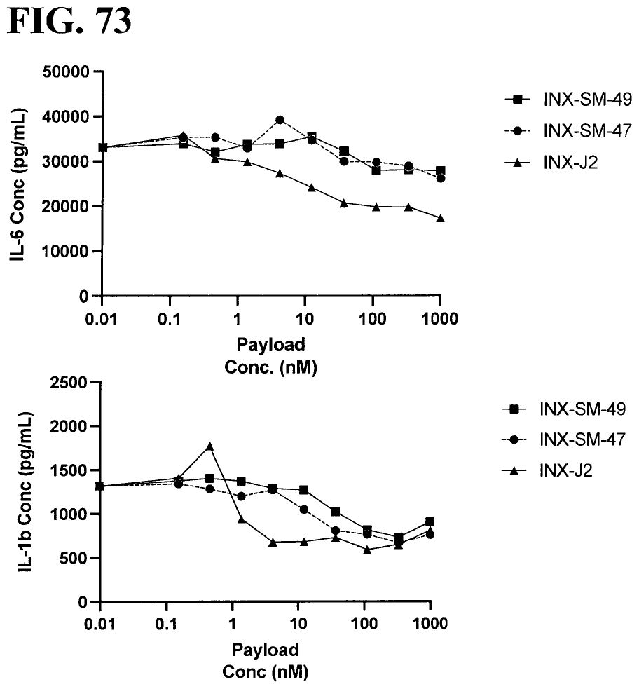

Also, the data in FIG. 73 show that INX-SM-49 and INX-SM-47 weakly inhibit IL-1β (top) and IL-6 (bottom) relative to INX-J2. Cytokine levels (IL1-β and IL-6) measured at 24 hr for human PBMC incubated with 1 ng/mL LPS and serial dilutions (1000-0.15 nM) of steroid payloads, with the no treatment control plotted on the log-scale x-axis at 0.01 nM; n=1 donor, mean of technical duplicates plotted.

### Experiment 13: Assessment of Anti-Inflammatory Potency of Novel Glucocorticoid Payloads in Full Conjugated Form (INX234J, INX201J, INX234A9, INX201V)

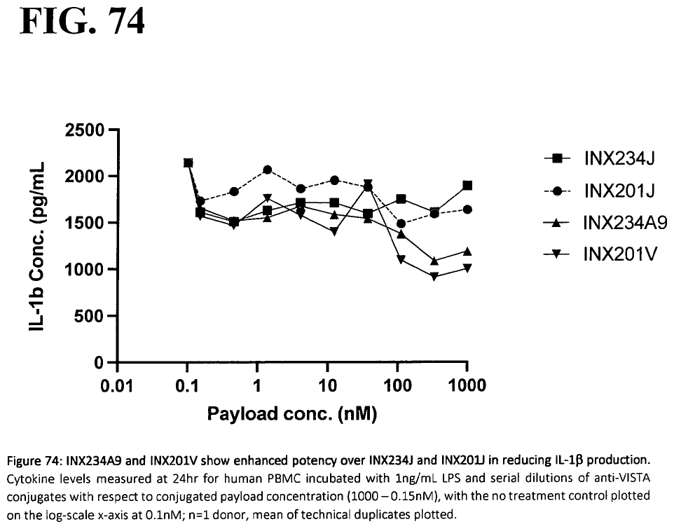

In this study, we evaluated the anti-inflammatory potency of novel glucocorticoid linker payloads in full conjugated form. INX-J2, a previously reported linker payload, was conjugated to INX234 and INX201 and compared to INX A9 conjugated to INX231 and INX V conjugated to INX201. PBMC from one donor was tested. As shown in FIG. 74, INX A9 and INX V show enhanced potency over INX J conjugated to the same antibody backbone. Of note, INX A9 includes a spirocyclic ring system that is different from that of INX V in the adaptor region of the payload and shows enhanced potency of the conjugated form similar to INX V over what would have been predicted by the free payload (INX-SM-46 shown in FIG. 118A-O). This is likely due to the enhanced accumulation observed with INX V.

Particularly the data in FIG. 74 show that INX231A9 and INX201V show enhanced potency over INX234J and INX201J in reducing IL-1β production. Cytokine levels measured at 24 hr for human PBMC incubated with 1 ng/ml LPS and serial dilutions of anti-VISTA conjugates with respect to conjugated payload concentration (1000-0.15 nM), with the no treatment control plotted on the log-scale x-axis at 0.1 nM; n=1 donor, mean of technical duplicates plotted.

### Experiment 14: Assessment of Anti-Inflammatory Potency of Novel Glucocorticoid Payloads in Full Conjugated Form (INX231V, INX231J, INX201A23, INX201V, INX201J, INX201P) (DAR 4 VS DAR 8)

In this study, we evaluated the anti-inflammatory potency of novel glucocorticoid payloads in full conjugated form, specifically assessing the impact of reduced DAR (˜4 vs ˜8). The conjugates INX231V (DAR 3.66), INX201A23 (DAR 4.34 and 7.71), INX201V (DAR 3.95 and 7.86), and INX201P (DAR 4.52) were assessed against the previously reported linker/payload, INX J (INX-J2) conjugated to INX201 and INX231 both at DAR 8.0. PBMCs from one donor was tested.

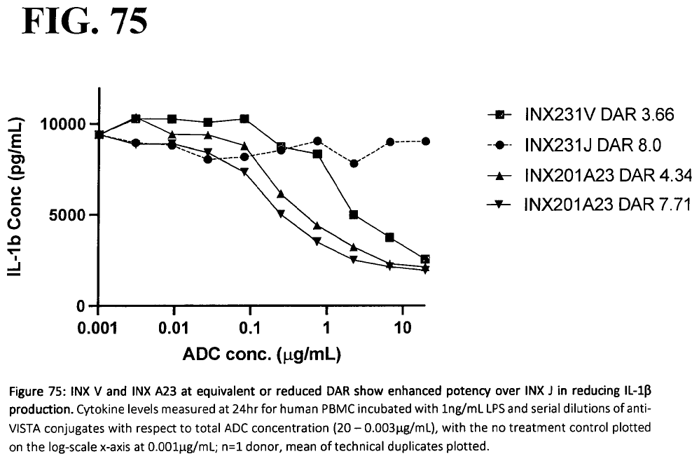

As shown in FIG. 75, the INX V conjugate at reduced DAR (3.66) has dramatically more potency in reducing IL-1β than INX J conjugate with the same antibody backbone at DAR 8.0. Similarly, INX201A23 which includes a similar payload to INX V, but with C6, C9 fluorination has increased potency at both DAR 7.71 and DAR 4.43 over both INX231V DAR 3.66 and INX231J DAR 8.0. Particularly, the data in FIG. 75 show that INX V and INX A23 at equivalent or reduced DAR show enhanced potency over INX J in reducing IL-1β production. Cytokine levels measured at 24 hr for human PBMC incubated with 1 ng/ml LPS and serial dilutions of anti-VISTA conjugates with respect to total ADC concentration (20-0.003 μg/mL), with the no treatment control plotted on the log-scale x-axis at 0.001 μg/mL; n=1 donor, mean of technical duplicates plotted.

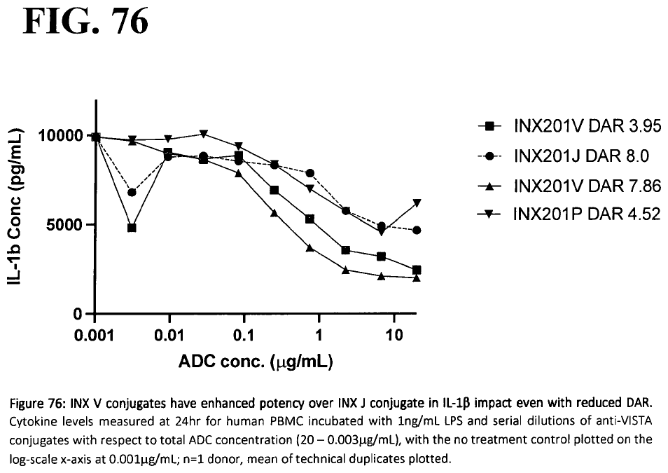

The experiment in FIG. 76 further shows that INX201V at DAR 7.86 or the reduced DAR of 3.95 demonstrate potency over INX201J DAR 8.0. Of note, INX201P (DAR 4.52) has similar potency to INX201J (DAR 8.0), even though it has a DAR 43% lower than that of the comparable INX J conjugate. For both INX201V conjugates and INX201A23 conjugates, the DAR ˜8 conjugate had approximately 2x the potency of the DAR ˜4 conjugate. Given the potency profile of these and other novel linker payloads described here, DAR 4 may be sufficient for therapeutic use, and may have favorable developability properties.

More particularly the data in FIG. 76 show that INX V conjugates have enhanced potency over INX J conjugate in IL-1β impact even with reduced DAR. In the experiments cytokine levels measured at 24 hr for human PBMC incubated with 1 ng/ml LPS and serial dilutions of anti-VISTA conjugates with respect to total ADC concentration (20-0.003 μg/mL), with the no treatment control plotted on the log-scale x-axis at 0.001 μg/mL; n=1 donor, mean of technical duplicates plotted.

### Conclusions

### Conclusions Relating to Novel Glucocorticosteroids

We have shown that the following novel GCs show varying degrees of dose dependent steroid potency as free payloads in an in vitro assay using LPS-activated human PBMC:
INX-SM-1, INX-SM-2, INX-SM-3, INX-SM-4, INX-SM-6, INX-SM-7, INX-SM-9,
INX-SM-10, INX-SM-13, INX-SM-24, INX-SM-31, INX-SM-32, INX-SM-33,
INX-SM-35, INX-SM-36, INX-SM-37, INX-SM-43, INX-SM-44, INX-SM-45,
INX-SM-46, INX-SM-74, INX-SM-14, INX-SM-15, INX-SM-17, INX-SM-34,
INX-SM-40, INX-SM-47, INX-SM-49

The lowest potency among the R stereoisomers of the series was observed with INX-SM-31, INX-SM-33, and INX-SM-37.

An assessment of the impact of fluorination on the potency of INX-SM-3 demonstrated that double halogenation at both the C6 and C9 positions of INX-SM-24 lead to increased potency. However, fluorination at the C9 position alone (INX-SM-13) did not lead to increased potency over the non-fluorinated payload (INX-SM-3)

The payloads containing the S stereoisomer at the acetal position—INX-SM-53, INX-SM-54, and INX-SM-56—did not show potency. The exception to this is INX-SM-74, which is halogenated at both the C9 and C6 position which showed moderate potency albeit much weaker than the R stereoisomer with the same halogenation.

Altering the acetal to a pyrrolidine (e.g., converting INX-SM-32 to INX-SM-37) also dramatically lowered the potency of the free payload.

### Conclusions Relating to Conjugated Linker Payloads

The data show that:
In their conjugated forms, the majority of linker payloads retain the potency of the free payload.
Linker payloads with a spiro[3.3]heptane (INX V series) had a potency enhancement over a literature precedent linker payload (INX J) to a level not predicted by the potency of the free payload. This potency enhancement may be due to enhanced release leading to higher levels of accumulation as seen in a prior example.
These linker payloads with enhanced potency included: INX V, INX A11, INX A3, INX A4, INX A5, INX A12, INX A7, INX A23, and INX A9.
Potency enhancement of conjugates is preserved with DAR ˜4 albeit total potency is approximately half of the corresponding DAR ˜8 conjugate as would be reasonably expected from the ˜50% reduction in payload per antibody. Even with the reduction in potency, a DAR of 4 may be sufficient for therapeutic use, and may have more desirable developability properties over DAR 8 molecules.
A separate linker payload with a spiro[2.5]octane also showed potency enhancement over INX J to a level not predicted by the potency of the free payload, INX-SM-46.
INX P conjugates had a modest potency increase over INX J conjugates with the same antibody backbone also potentially due to enhanced release leading to higher levels of accumulation. This potency increase results in an INX P conjugate with a 43% lower DAR having equivalent potency to the INX J conjugate with the same antibody backbone.
INX201 conjugates with the same linker payload had increased potency over INX231 conjugates. INX234 and INX231 conjugates with the same linker payload have similar potency.
INX P conjugates directed against other cell surface targets (e.g. anti Target A-P, anti-Target B—P) also had potency in this assay, confirming that conjugation to specifically an anti-VISTA target antibody is not required for the linker payload to have potency.

The data further show that:
The steroid structure is able to accommodate a variety of alternate geometries, ring sizes and structures off of the C17/C16 acetal while maintaining potency.
However, only the R isomer at the acetal carbon of the non-halogenated steroid is tolerated. Of note, the presence of fluorination at both position C6 and C9 on the steroid ring does allow for potency of the S isomer, albeit much weaker than the corresponding R isomer.
R isomers: INX-SM-1, INX-SM-2, INX-SM-3, INX-SM-4, INX-SM-6, INX-SM-7, INX-SM-09, INX-SM-10, INX-SM-13, INX-SM-24, INX-SM-31, INX-SM-32, INX-SM-33, INX-SM-35, INX-SM-43, INX-SM-44, INX-SM-45, INX-SM-46, INX-SM-36.
S isomers: INX-SM-53, INX-SM-54, INX-SM-56, INX-SM-74 (fluorinated at C6/C9)
Pyrrolidine analogs (INX-SM-37) in place of the acetal are less potent but still maintain some potency
Conjugated forms of some payloads, specifically INX V with the INX-SM-32 payload and its analogs, allow substantial enhancement of efficacy potentially due to enhanced release and exposure of the corresponding free payload.
When the antibody is held constant, the conjugated form of INX P has a modest enhancement of potency over INX J conjugates even though the free payload form of INX J (INX-SM-J2) may be modestly more potent than that of INX P (INX-SM-3)

### Example 10: Pharmacokinetic Evaluation of Exemplary Anti-VISTA Antibodies

In this example studies were conducted to define the pharmacokinetics (PK) of various anti-human VISTA antibodies according to the invention and compare same with the pH sensitive anti-human VISTA from BMS (767-IgG1.3, Johnston et al, 2019).

The objective of the present experiment was to 1) confirm that the “pH sensitive” antibody described by BMS/Five Prime Therapeutics has a significantly different PK (comparable to hlgG1) compared to ImmuNext (INX) anti-VISTA antibodies; 2) evaluate the PK of a larger number of INX anti-VISTA antibodies. (See INX200 and other INX antibody sequences in FIGS. 8, 10 and 12); and to further evaluate the PK of a larger number of other anti-VISTA antibodies (See other INX antibody sequences in FIGS. 8, 10 and 12).

These studies were conducted in human VISTA knock-in (hVISTA KI) mice which mice have the human VISTA cDNA knocked-in in place of the mouse VISTA gene, and express human VISTA both at RNA and protein levels. The experiments were performed in female or male hVISTA KI mice and in all studies the animals received one dose of antibody at 10 mg/Kg. Antibody amount in peripheral blood was quantified by ELISA.

### Materials and Methods

### Experiment Design

### Experiment 1

hVISTA KI mice were divided into 2 groups of 10 mice each, treated respectively with one dose of human IgG1 and INX200 at 10 mg/Kg on day 0.

### Experiment 2

hVISTA KI mice were divided into 2 groups of 10 mice each, treated respectively with one dose of human IgG1 and 767-IgG1.3 at 10 mg/Kg on day 0.

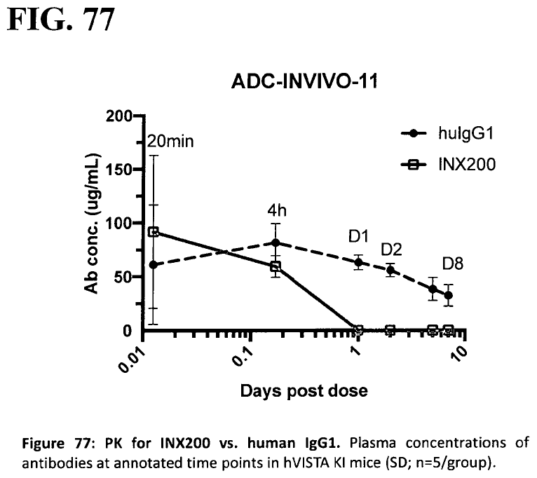

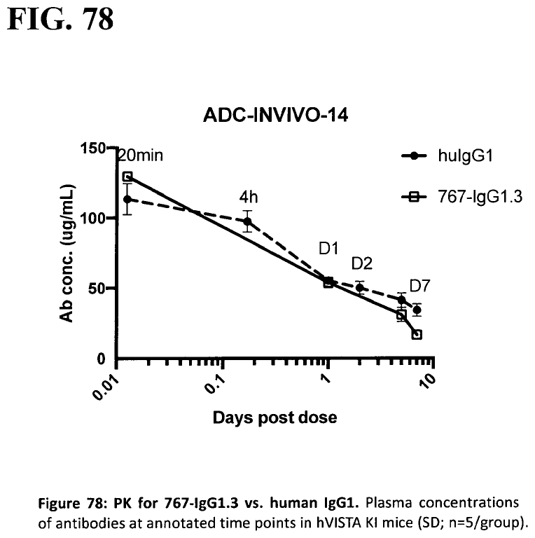

In both EXPERIMENT 1 and EXPERIMENT 2 five mice were bled retro-orbitally at 20 min, 4, 24, 48 hrs, and then at day 5 and 8 for EXPERIMENT 1 and day 4 and 7 for EXPERIMENT 2; circulating antibodies were quantified by ELISA. These results are respectively in FIG. 77 and FIG. 78.

### Experiment 3

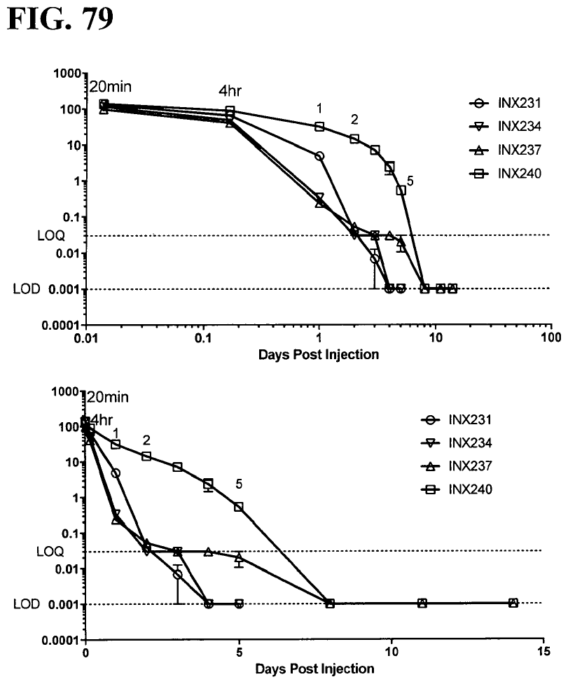

hVISTA KI mice were divided into 4 groups of 15 mice each, treated respectively with one dose of INX231, INX234, INX237 and INX240 at 10 mg/Kg on day 0. Five mice per group were bled retro-orbitally at 20 min, 4 h, 24 h and then on day 2, 3, 4, 5, 8, 11, 14 and 21. These results are in FIG. 79.

### Experiment 4

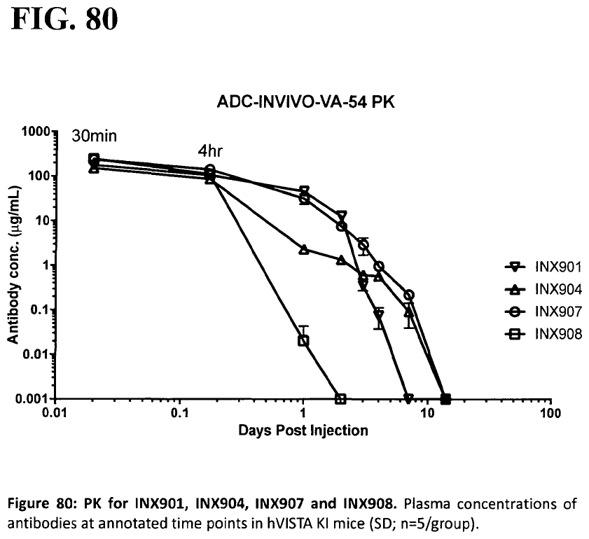

hVISTA KI mice were divided into 4 groups of 10 mice each, treated respectively with one dose of INX901, INX904, INX907 and INX908 at 10 mg/Kg on day 0. Five mice per group were bled retro-orbitally at 30 min, 4 h, 24 h and then on day 2, 3, 4, 7 and 14. These results are in FIG. 80.

### Experiment 5

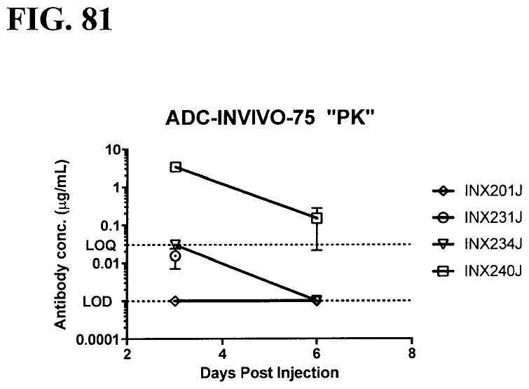

hVISTA KI mice were divided into 5 groups of 4 mice each, treated respectively with one dose of INX201J, INX231J, INX234J, and INX240 J at 10 mg/Kg on day 0. The mice were bled retro-orbitally on day 3 and 6. These results are in FIG. 81.

### Test Agents and Dosage

INX200 (Aragen, Lot #BP-2875-019-6.1) is a humanized anti-human VISTA antibody on a human IgG1/kappa backbone with L234A/L235A silencing mutations in the Fc region.
INX201 (Aragen, Lot #BP-3200-019-6) is a humanized anti-human VISTA antibody (same variable domains as INX200) on a human IgG1/kappa backbone with L234A/L235A/E269R/K322A silencing mutations in the Fc region.
Human IgG1 (BioXcell ref, Lot #659518N1)
767-IgG1,3 (Aragen, Lot #BP-2985-019-6) is an anti-human VISTA antibody developed by Five Prime Therapeutics and Bristol-Myers Squibb Company on a human IgG1/kappa backbone with L234A/L235E/G237A silencing mutations in the Fc region. This antibody was designed to bind at low pH (e.g. pH 6) but to have minimal binding at physiological pH (pH 7.4).
INX231, INX234, INX237 and INX240 (lot #72928.1.a, 72931.1.a, 72934.1.a and 73419.1.a respectively) are humanized anti-human VISTA antibody on a human IgG1/kappa backbone with L234A/L235A/E269R/K322A silencing mutations in the Fc region.
INX201J, INX231J, INX234J and INX240J (lot #JZ-0556-027, JZ-0556-013-1, JZ-0556-013-2, JZ-0556-013-3) are respectively INX201, INX231, INX234, INX237 and INX240 with a drug/antibody ratio (DAR) of 8.0, conjugated via full modification of the interchain disulfides. The linker/payload (J) is based on a patent reported linker/payload that consists of a protease sensitive linker with a budesonide analog payload.
INX901, INX904, INX907 and INX908 are humanized anti-human VISTA antibodies on a native human IgG2/kappa backbone, where the variable domains match INX231, INX234, INX237 and INX200/INX201, respectively.

All antibodies were diluted in PBS and injected intravenously in the mouse tail vein in a volume of 0.2 ml to deliver a dose of 10 mg/Kg.

### Mice

The hVISTA mice were bred at Sage Labs (Boyertown, PA). The mice, aged 8-12 weeks, first transited for 3 weeks in our quarantine facility, and then were transferred to the regular facility. They were acclimated for 1 to 2 weeks prior to experiment initiation.

### Blood Draw and Preparation

Animals were bled no more than once every 24 hrs. Each mouse group was divided in 2 or 3 sub-groups of 5 mice that were bled alternatively on day 0. Blood was collected on day 0 post injection at 20 min, 4, 24, 48 hrs, and then at day 5 and 8 for EXPERIMENT 1 and day 4 and 7 for EXPERIMENT 2. In the first 24 hrs period, some data were excluded based on the registered quality of the intravenous injections. For subsequent time points, only animals that had successful intravenous injections were bled.

For EXPERIMENT 3, mice were bled at 20 min, 4 h, 24 h and then on day 2, 3, 4, 5, 8, 11, 14 and 21.

For EXPERIMENT 4 and 5, mice were bled at 30 min, 4 h, 24h, and then on day 2, 3, 4, 7, 14.

Peripheral blood was harvested from the retro-orbital cavity using a glass Pasteur pipette that was first rinsed with heparin to prevent coagulation. Blood was then centrifuged at 400 rcf for 5 min and plasma collected and stored at −80° C. for analysis (See infra).

### Antibody Blood Concentration Analysis

### ELISA for Detection of Human IgG1

First, 96-well flat-bottom plates (Thermo Scientific Nunc Immunol Maxisorp, cat #442404) were coated with mouse anti-huIgG Fcγ (Jackson ImmunoResearch, cat #209-005-098) at 1 μg/ml in PBS for one hour at room temperature (RT).

The wells were washed 3 times with PT (PBS with 0.05% Tween 20) then blocked with PTB (PBS with 0.05% Tween 20 and 1% BSA) for 1 hour at RT. Human IgG (Southern Biotech, cat #0150-01) was used as a positive control and human IgG1 (BioXcell, cat #BE0297) was used to build a standard curve. The wells were washed 3 times with PT then plasma samples were incubated at up to 4 different dilutions in PTB (to fit on the standard curve) for 1 hour at RT.

After 3 washes with PT, mouse anti-human IgG Fcγ coupled to HRP (Jackson ImmunoResearch, cat #209-035-098), was used as detection reagent at a dilution of 1/2000 and incubated for 1 hour at RT. Following 3 washes, the ELISA reaction was revealed using TMB (Thermo Scientific, cat #34028) as a colorimetric substrate. After 5-10 min at RT, the reaction was stopped with 1M H₂SO₄.

### ELISA Detection of INX200 (Experiment 1)

First, 96-well flat-bottom plates (same as in 4.5.1) were coated with hIX50 (human VISTA ECD, produced at Aragen Bioscience for ImmuNext) at 1 μg/ml in PBS for one hour at RT.

After 3 washes, the wells were blocked with PTB for one hour at RT. INX908 (produced at Aragen Bioscience for ImmuNext) was used as a positive control and INX200 was used to build a standard curve. The wells were washed 3 times with PT then plasma samples were incubated at up to 4 different dilutions in PTB (to fit on the standard curve) for 1 hour at RT.

After 3 washes with PT, mouse anti-human Kappa-HRP (Southern Biotech, cat #9230-05) was used at 1/2000 as a detection reagent, incubating 1 hour at RT. Following 3 washes, the ELISA reaction was revealed using TMB substrate. After 5 min at RT, the reaction was stopped with 1M H2S04.

### ELISA Detection of 767-IgG1.3 (Experiment 2)

First, 96-well flat-bottom plates (same as above) were coated with mouse anti-huIgG Fcγ (Jackson ImmunoResearch, cat #209-005-098) at 1 μg/ml in PBS for one hour at RT.

After 3 washes, the wells were blocked with PTB for one hour at RT. Human IgG (Southern Biotech, cat #0150-01) was used as a positive control and 767-IgG1.3 was used to build a standard curve. The wells were washed 3 times with PT then plasma samples were incubated at up to 4 different dilutions in PTB (to fit on the standard curve) for 1 hour at RT.

After 3 washes in PTB, mouse anti-human IgG Fcγ-HRP (Jackson ImmunoResearch, cat #209-035-098) was used at 1/2000 as a detection reagent, incubating 1 hour at RT. Following 3 washes, the ELISA reaction was revealed using TMB substrate following manufacturer instructions. After 5 min at RT, the reaction was stopped with 1M H₂SO₄.

### ELISA for Experiment 3

First, 96-well flat-bottom plates (same as above) were coated with hIX50 (human VISTA ECD, produced at Aragen Bioscience for ImmuNext) at 1 mg/ml in PBS for one hour at RT.

After 3 washes, the wells were blocked with PTB for one hour at RT. INX908 (produced at Aragen Bioscience for ImmuNext) was used as a positive control and INX231, INX234, INX237 or INX240 were used to build a standard curve. The wells were washed 3 times with PT then plasma samples were incubated at up to 4 different dilutions in PTB (to fit on the standard curve) for 1 hour at RT.

After 3 washes in PTB, mouse anti-human IgG Fcγ-HRP (Jackson ImmunoResearch, cat #209-035-098) was used at 1/2000 as a detection reagent, incubating 1 hour at RT. Following 3 washes, the ELISA reaction was revealed using TMB substrate following manufacturer instructions. After 5 min at RT, the reaction was stopped with 1M H₂SO₄.

### ELISA for Experiment 4

First, 96-well flat-bottom plates (same as prior Experiment) were coated with hINX50 (human VISTA ECD, produced at Aragen Bioscience for ImmuNext) at 1 mg/ml in PBS for one hour at RT.

After 3 washes, the wells were blocked with PTB for one hour at RT. INX201 was used as a positive control and INX231, INX234 or INX240 were used to build a standard curve. The wells were washed 3 times with PT then plasma samples were incubated at up to 4 different dilutions in PTB (to fit on the standard curve) for 1 hour at RT.

After 3 washes in PTB, mouse anti-human IgG Fcγ-HRP (Jackson ImmunoResearch, cat #209-035-098) was used at 1/2000 as a detection reagent, incubating 1 hour at RT. Following 3 washes, the ELISA reaction was revealed using TMB substrate following manufacturer instructions. After 5 min at RT, the reaction was stopped with 1M H₂SO₄.

### ELISA for Experiment 5

First, 96-well flat-bottom plates (same as above) were coated with hIX7 (human VISTA ECD on a mouse IgG2s backbone) at 1 mg/ml in PBS for one hour at RT.

After 3 washes, the wells were blocked with PTB for one hour at RT. INX901, INX904, INX907 or INX908 were used as positive controls and to build a standard curve. The wells were washed 3 times with PT then plasma samples were incubated at up to 4 different dilutions in PTB (to fit on the standard curve) for 1 hour at RT.

After 3 washes in PTB, mouse anti-human IgG Fcγ-HRP (Jackson ImmunoResearch, cat #209-035-098) was used at 1/2000 as a detection reagent, incubating 1 hour at RT. Following 3 washes, the ELISA reaction was revealed using TMB substrate following manufacturer instructions. After 5 min at RT, the reaction was stopped with 1M H₂SO₄.

### ELISA Assay Calculations

LOQ is calculated by multiplying the lowest point of the standard curve by the lowest dilution factor used to dilute the sample. For example, if the lowest standard point is 0.3 ng/ml and the lowest standard dilution is 1/400, then the LOQ is 0.1 μg/mL as it is reported in the same units as the sample is reported.

The LOD is determined when the sample OD cannot be distinguished from the background OD, approximately an OD of 0.01. No concentration is calculated for the LOD but a concentration of 0 or 0.001 μg/mL is assigned for graphing and PK calculation purposes.

Antibody half-life was determined using the PKsolver program performing a non-compartmental analysis (NCA) after intravenous bolus.

The results of EXPERIMENTS 1-5 are respectively in FIGS. 77-81.

FIG. 77 contains the results of EXPERIMENT 1 comparing the PK for INX200 vs. human IgG1. Plasma concentrations of antibodies at annotated time points in hVISTA KI mice (SD; n=5/group) are shown.

FIG. 78 contains the results of EXPERIMENT 2 comparing the PK of 767-IgG1.3 vs. human IgG1. Plasma concentrations of antibodies at annotated time points in hVISTA KI mice (SD; n=5/group) are shown.

FIG. 79 contains the results of EXPERIMENT 3 comparing the PK for INX231, INX234, INX237 and INX240. Plasma concentrations of antibodies at annotated time points in hVISTA KI mice (SD; n=5/group) are shown. Left graph shows y and x axes in Log 10, while for right graph, only the y axis is in Log 10.

FIG. 80 contains the results of EXPERIMENT 4 comparing the PK for INX901, INX904, INX907 and INX908. Plasma concentrations of antibodies at annotated time points in hVISTA KI mice (SD; n=5/group) are shown.

FIG. 81 contains the results of EXPERIMENT 5 comparing the PK for INX201J, INX231J, INX234J and INX240J. Plasma concentrations of antibodies at annotated time points in hVISTA KI mice (SD; n=4/group) are shown.

The data in these experiments show the following:

EXPERIMENT 1 (FIG. 77) shows that the PK for anti-human VISTA antibody INX200 is not quantifiable in plasma at 24 hrs post dosing due to target mediated drug disposition (TMDD) while the human IgG1 control shows the more typical extended half-life for an IgG.

EXPERIMENT 2 (FIG. 78) shows that the pH sensitive anti-human VISTA 767-IgG1,3 exhibits a PK similar to the human IgG1 control antibody suggesting that it is has limited binding to its VISTA target and is not subjected to TMDD.

EXPERIMENT 3 (FIG. 79) results show that INX231, INX234, INX237 and INX240 are all still detectable after 24 hr and that INX237 has a distinctively increased half-life.

EXPERIMENT 4 (FIG. 80) results show that that the incorporation of different IgG backbones onto the inventive INX antibodies did not appreciably change antibody half-life.

EXPERIMENT 5 (FIG. 81) results show that that the addition of a GC payload further did not appear to affect the clearance of INX anti-VISTA antibodies at the 2 time points analyzed.

These results indicate that the inventive anti-VISTA antibodies and ADCs containing same possess PK values and clearance properties making them well suited for targeted delivery of desired payloads, particularly steroid payloads into target immune cells.

### Example 11: Impact of Long-Term Treatment with INX201J Vs. Dexamethasone on Corticosterone Levels

Glucocorticoid hormones are rapidly synthesized and secreted from the adrenal gland in response to stress. In addition, under basal conditions glucocorticoids are released rhythmically with both a circadian and an ultradian (pulsatile) pattern. These rhythms are important not only for normal function of glucocorticoid target organs, but also for the HPA axis responses to stress. Numerous studies have shown that disruption of glucocorticoid rhythms by prolonged GC treatment is associated with disease both in humans and in rodents. In human, the most abundant GC is cortisol, in mice, it is corticosterone.

Based on the foregoing we assessed the impact of long-term treatment with an exemplary antibody drug conjugate (ADC) INX201J, and an anti-human VISTA monoclonal antibody linked to a glucocorticoid (GC) payload, on the HPA axis, specifically the corticosterone basal levels. As discussed below the experiment was conducted in human VISTA knock-in (hVISTA KI) which have the human VISTA cDNA knocked-in in place of the mouse VISTA gene, and express human VISTA both at RNA and protein levels with the same expression pattern as mouse VISTA. The experiment was performed in female mice that were first acclimated for a week to a specific handler that would carry subsequently all injections and bleed to limit stress-induced changes in basal level of GC.

Mice were then subjected to injections of INX201J at 10 or 3 mg/Kg (0.2 or 0.06 mg/Kg of payload respectively) or dexamethasone (Dex) at 2 or 0.2 mg/Kg. Dex was dosed daily for 4 days while INX201J was dosed on days 1, 3 and 4. On day 5, mice were bled and their corticosterone levels assessed by ELISA.

### Materials and Methods

### Experiment Design

The experiment was performed in female hVISTA KI mice. Mice were then subjected to injections of INX201J at 10 or 3 mg/Kg (0.2 or 0.06 mg/Kg of payload respectively) on day 1, 3 and 4; or daily injection of dexamethasone (Dex) at 2 or 0.2 mg/Kg for 4 days in a row. A control group was included that received daily PBS injections. On day 5, mice were bled and their plasma corticosterone levels assessed by ELISA.

The rationale for the dosing schedule is based on other studies (See previous example) that showed that the inventive ADC has a much longer pharmacodynamic range (>96h) than Dex (<24h).

### Experiments: (8 Mice Per Group)

Group 1: PBS
Group 2: Dex 2 mg/Kg
Group 3: Dex 0.2 mg/Kg
Group 4: INX201J 10 mg/Kg (0.2 mg/Kg of payload)
Group 5: INX201J 3 mg/Kg (0.06 mg/Kg of payload)

### Test Agents and Dosage

### Antibodies

INX201J (Abzena, Lot #s: JZ-0556-025-1, JZ-0556-027, JZ-0556-013) is based on INX201 which is a humanized anti-human VISTA antibody on a human IgG1/kappa backbone with L234A/L235A/E269R/K322A silencing mutations in the Fc region. INX201J is the conjugated antibody with a drug/antibody ratio of 8.0, conjugated via full modification of the interchain disulfides. The linker/payload (J) consists of a protease sensitive linker with a budesonide analog payload.

The antibodies were diluted in PBS and injected intraperitoneally (i.p.) in a volume of 0.2 ml to deliver a specified dose.

### Dexamethasone

Dexamethasone sterile injection from Phoenix, NDC 57319-519-05, was diluted in PBS and dosed as described via i.p. injection.

### Mice

The hVISTA mice were bred on site (Center for Comparative Medicine and Research at Dartmouth). All the experiments were done in female mice enrolled between 9 and 15 weeks of age.

### Blood Draw and Preparation

Peripheral blood was harvested from the retro-orbital cavity using a glass Pasteur pipette that was first rinsed with heparin to prevent coagulation. Blood was then centrifuged at 550 rcf for 5 min and plasma collected and stored at −80° C. before corticosterone analysis.

### Corticosterone ELISA

The ELISA was conducted following the manufacturer's included protocol using Arbor Assays (cat #K014-H5) Corticosterone 5 pack ELISA Kit.

### Results

### INX201J has Limited Impact on Corticosterone Levels

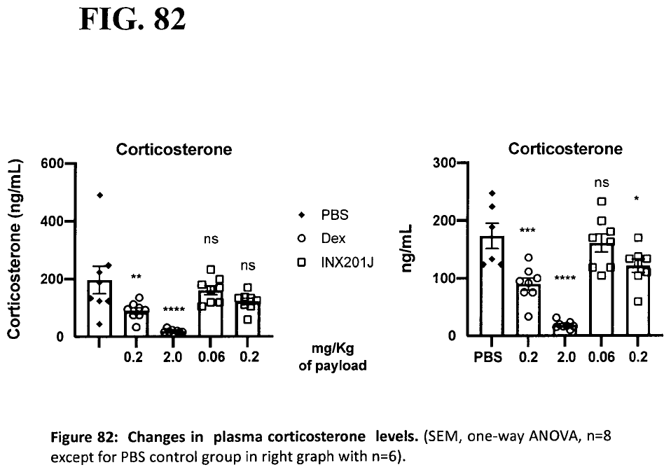

As shown in the experiment in FIG. 82 mouse acclimation led to relatively consistent levels of corticosterone in the control group except for 2 animals, one showing very high and the other very low corticosterone levels. FIG. 82 shows the uncensored data on the left and censored (without the 2 outliers in the control group) data on the right and shows changes in plasma corticosterone levels. (SEM, one-way ANOVA, n=8 except for PBS control group in right graph with n=6). As shown Dex at 2 mg/Kg dramatically decreased basal corticosterone levels but at only limited though significant impact at 0.2 mg/Kg. By contrast, INX201J had no impact at a dose of 0.06 mg/Kg of payload but a limited decrease was observed at 0.2 mg/Kg of payload.

### Conclusions

The data show that Dex at 2 mg/Kg dramatically reduces basal levels of corticosterone while at 0.2 mg/Kg the decrease is more limited though still highly significant (P<0.001). By contrast, INX201J at 0.2 mg/Kg of payload, which is therapeutically equivalent to 2 mg/Kg Dex, had a more limited impact (ns or P<0.5). At 0.06 mg/Kg, there was no effect on corticosterone levels. (These doses were selected because as shown in the previous examples INX201J at 0.2 mg/Kg of payload has similar efficacy as Dex at 2 mg/Kg).

### Example 12: Impact of ADCs on Antigen Specific Responses

Glucocorticoids (GC) are known to have profound effects on primary immune responses and can significantly affect IgG responses to vaccines. Accordingly, we used a vaccine model to evaluate the functionality of the subject antibody drug conjugates (ADCs) in disrupting antigen-specific responses. As discussed in detail below we used a standard immunization protocol combining a mouse CD40 agonist antibody (FGK4.5), the OVA peptide SIINFEKL as a model antigen (Ag) and the TLR agonist Poly (I:C), which drives a potent CD8 T cell driven Ag response that can be measured using the tetramer technology. A further benefit is that this model permitted us to also evaluate the pharmacodynamic range of our ADCs by treating up to 1 week pre vaccine inoculation.

As discussed in detail below three studies were conducted in such human VISTA knock-in mice (hVISTA KI), which have the human VISTA cDNA knocked-in in place of the mouse VISTA gene, and express human VISTA both at RNA and protein levels with the same expression pattern as mouse VISTA. In brief these mice were injected with ADCs up to 7 days pre-immunization. Dexamethasone (Dex) was used as a positive GC control. Immune response in peripheral blood was measured on day 6 post immunization at the peak of the anti-Ag response.

### Materials and Methods

### Experiment Design

All 4 experiments were performed in female mice with 5 mice per group.

### Experiment 1: Impact of Dex on Ag-Specific Response when Administered 2h Pre-Immunization

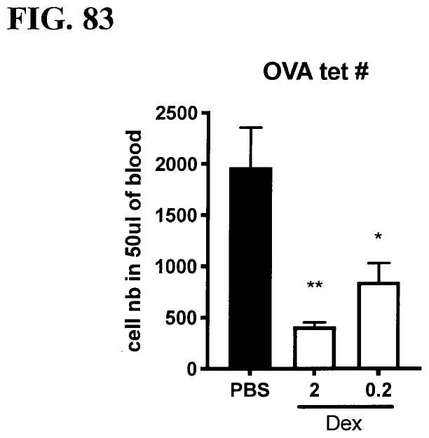

The experiment shown in FIG. 83 was done to confirm the impact of Dex on Ag-specific response when administered 2h pre-immunization and was conducted in C57BI/6 mice.
Group 1: PBS
Group 2: Dex at 2 mg/Kg
Group 3: Dex at 0.2 mg/Kg

Mice were dosed i.p. with Dex at 2 or 0.2 mg/Kg or PBS. Two hours later, they received the vaccine cocktail injected i.p. These mice were then bled after 6 days and Ag specific CD8 T cells number quantified.

### Experiment 2: Impact of the ADC INX201J on Ag-Specific Response when Administered at Different Time Points Pre-Immunization

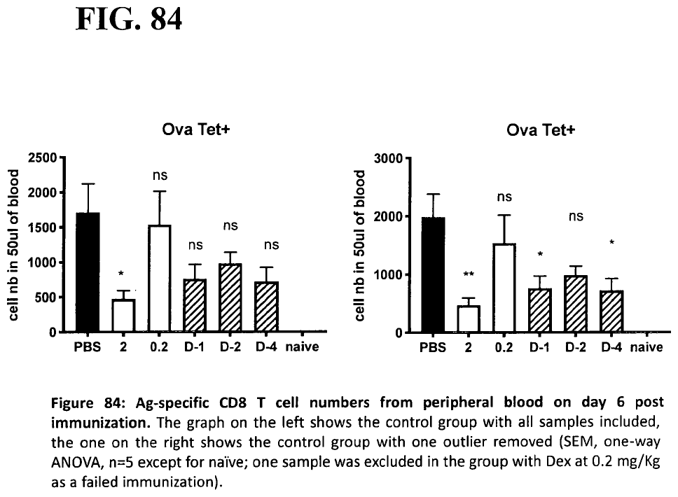

This experiment in FIG. 84 was done to evaluate the impact of the ADC INX201J on Ag-specific response when administered at different time points pre-immunization and was conducted in hVISTA KI mice.
Group 1: PBS
Group 2: Dex at 2 mg/Kg
Group 3: Dex at 0.2 mg/Kg
Group 4: INX201J at 10 mg/Kg-d-1
Group 5: INX201J at 10 mg/Kg-d-2
Group 6: INX201J at 10 mg/Kg-d-4

Mice from groups 1 to 3 were dosed i.p. 2h before immunization. Mice from groups 4 to 6 were dosed as indicated 1, 2 or 4 days pre-immunization. All mice were immunized on day 0. All of these animals were then bled after 6 days and Ag specific CD8 T cells number quantified.

### Experiment 3

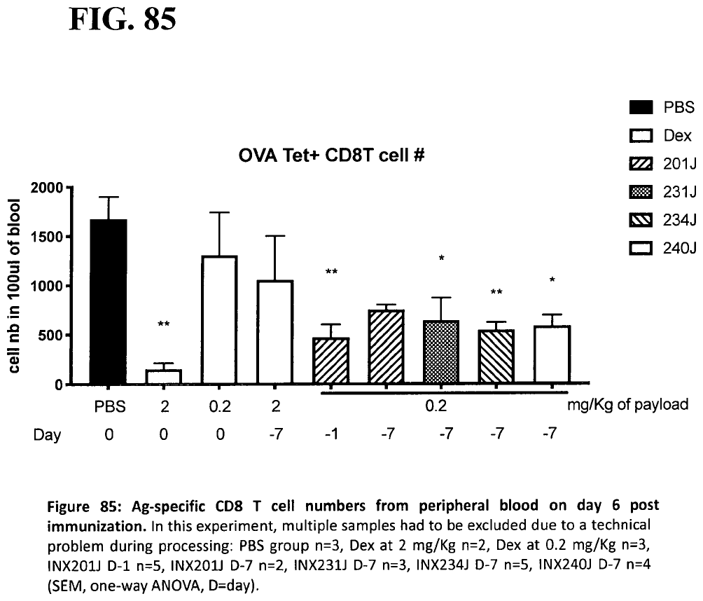

This experiment in FIG. 85 was done to evaluate the impact of multiple ADCs on Ag-specific response when administered at different time points pre-immunization and was conducted in hVISTA KI mice.
Group 1: PBS-2h pre-vaccine
Group 2: Dex at 2 mg/Kg-2h pre-vaccine
Group 3: Dex at 0.2 mg/Kg-2h pre-vaccine
Group 4: Dex at 2 mg/Kg-day −7
Group 5: INX201J at 10 mg/Kg-d-1
Group 6: INX201J at 10 mg/Kg-d-7
Group 7: INX231J at 10 mg/Kg-d-7
Group 8: INX234J at 10 mg/Kg-d-7
Group 9: INX240J at 10 mg/Kg-d-7

Mice from groups 1 to 3 were dosed i.p. 2h before immunization. Mice from groups 4 to 9 were dosed as indicated 1 or 7 days pre-immunization. All mice were immunized on day 0. All of these animals were then bled after 6 days and Ag specific CD8 T cells number quantified.

### Experiment 4: Impact of Multiple ADCs Conjugated to a GC Payload (P) on Ag-Specific Response

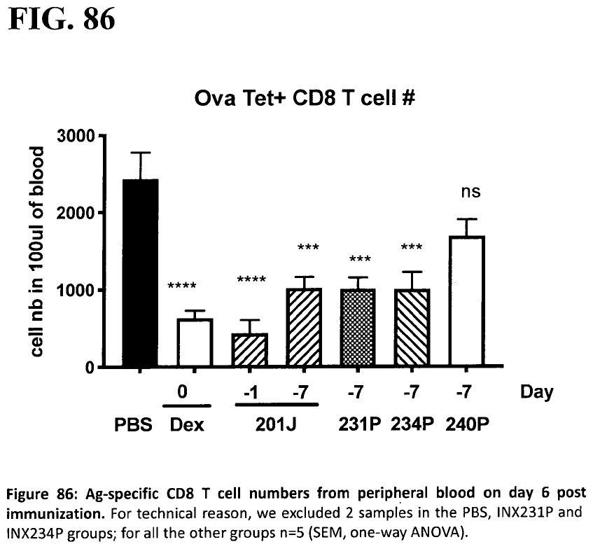

This experiment in FIG. 86 was done to evaluate the impact of multiple ADCs conjugated to a GC payload (P) on Ag-specific response when administered at different time points pre-immunization and was conducted in hVISTA KI mice.
Group 1: PBS-2h pre-vaccine
Group 2: Dex at 2 mg/Kg-2h pre-vaccine
Group 3: INX201J at 10 mg/Kg-d-1
Group 4: INX201J at 10 mg/Kg-d-7
Group 5: INX231P at 10 mg/Kg-d-7
Group 6: INX234P at 10 mg/Kg-d-7
Group 7: INX240P at 10 mg/Kg-d-7

Mice from groups 1 to 2 were dosed i.p. 2h before immunization. Mice from groups 3 to 7 were dosed as indicated 1 or 7 days pre-immunization. All mice were immunized on day 0. All of these animals were then bled after 6 days and Ag specific CD8 T cells number quantified.

### Test Agents and Dosage

### Antibodies

INX201, INX231, INX234 and INX240 (lot #72928.1.a, 72931.1.a and 73419.1.a respectively) were used in these experiments which all comprise humanized anti-human VISTA antibodies on a human IgG1/kappa backbone with L234A/L235A/E269R/K322A silencing mutations in the Fc region.

INX201J, INX231J, INX234J and INX240J (lot #JZ-0556-027, JZ-0556-013-1, JZ-0556-013-2, JZ-0556-013-3) respectively comprise INX201, INX231, INX234 and INX240 with a drug/antibody ratio (DAR) of 8.0, conjugated via full modification of the interchain disulfides. The linker/payload (J) consists of a protease sensitive linker with a budesonide analog payload.

INX201P, INX231P, INX234P and INX240 P (lot #JZ-0556-0271, JZ-0556-017-1, JZ-0556-017-2, JZ-0556-017-3) are respectively INX201, INX231, INX234 and INX240 with a drug/antibody ratio (DAR) of 8.0, conjugated via full modification of the interchain disulfides. The linker/payload (P) consists of a protease sensitive linker with a budesonide analog payload.

Each of these antibodies was diluted in PBS and injected intraperitoneally (i.p.) in a volume of 0.2 ml to deliver a specified dose.

### Dexamethasone

Dexamethasone sterile injection from Phoenix, NDC 57319-519-05, was diluted in PBS and dosed as described via i.p. injection.

### Vaccine Cocktail

A standard immunization protocol of antibody/peptide/poly (I:C) with 50 μg mouse CD40 agonist antibody (clone FGK4.5)+50 μg SIINFEKL peptide+50 μg poly (I:C) was used per mouse. Vaccine is diluted in PBS and injected i.p. in a final volume of 200 μl.

### Mice

The hVISTA mice were bred on site (Center for Comparative Medicine and Research at Dartmouth). All the experiments were done in female mice enrolled between 9 and 15 weeks of age. C57BI/6 mice were purchased from Jackson Laboratories.

### Blood Draw and Immunostaining

Peripheral blood was harvested from the retro-orbital cavity using a glass Pasteur pipette that was first rinsed with heparin to prevent coagulation. A 1-wash protocol was used that allows for absolute blood cell count.

10 μl of antibody cocktail (See below) was directly added to 50 or 100 μl of blood. After 30 min incubation at room temperature (RT), 600 μl BD FACS lysis buffer was added to the sample. After 30 min incubation at RT, samples were spun at 550 rcf for 5 min, wash once in PBS, resuspended in a fixed volume of PBS. The whole sample was run on a MacsQuant flow cytometer to obtain an absolute cell number.

### Antibody Panel:

The following antibodies were diluted in PBS.
CD11a-FITC (BioLegend; Clone 2D7; 0.5 mg/ml) 1:200
H-2 kb-OVA-PE tetramer (MBL iTag MHC Tetramer Cat #T03000; Lot #T1603004); (10 μl/sample)
CD8-Alexa647 (clone KT15; MBL #D271-A64; 1 mg/ml) (1:800).
Mouse Fc block (1:200)

### Gating Strategy:

FSC vs. SSC-Gating on lymphocyte population.
FSC—H vs. FSC-A-Singlet population
Gate on CD8+ T cells
CD8+→CD11a+vs Ova-tet+

### Results

### Experiment 1

As noted above the experiment in in FIG. 83 was done to confirm the impact of Dex on Ag-specific response when administered 2h pre-immunization and was conducted in C57BI/6 mice. Dex at 2 mg/Kg dramatically decreased the number of Ag-specific CD8 T cells (OVA tet) and clear reduction where also observed at 0.2 mg/Kg. Specifically, FIG. 83 shows Ag-specific CD8 T cell numbers from peripheral blood on day 6 post immunization. (SEM, one-way ANOVA, n=5).

### Experiment 2

As shown in FIG. 84, INX201J when dosed 24 h, 48h or 96h pre-immunization at 0.2 mg/Kg of GC payload shows similar efficacy as Dex dosed 2h pre-immunization at 2 mg/Kg in reducing Ag-specific (Ova Tet+ CD8 T cell) responses. In this experiment blood from 2 naïve mice was added on the last to provide a baseline control. More specifically, FIG. 84 shows Ag-specific CD8 T cell numbers from peripheral blood on day 6 post immunization. The graph on the left in the FIG. 84 shows the PBS control group with all samples included, the one on the right shows the PBS control group with one outlier removed (SEM, one-way ANOVA, n=5 except for naïve; one sample was excluded in the group with Dex at 0.2 mg/Kg as a failed immunization).

### Experiment 3

Four different ADCs were evaluated in the experiment in FIG. 85. As shown therein, all of the tested ADCs showed significant efficacy when dosed 1 or 7 days pre-immunization, at 0.2 mg/Kg of GC payload, in reducing Ag-specific (Ova Tet+ CD8 T cell) responses. It further can be seen therefrom that Dex showed efficacy when dosed at 2 mg/Kg but lost efficacy at 0.2 mg/Kg or when dosed at 2 mg/Kg 7 days pre-immunization. More specifically the experiment in FIG. 85 shows Ag-specific CD8 T cell numbers from peripheral blood on day 6 post immunization. In this experiment, multiple samples had to be excluded due to a technical problem during processing: PBS group n=3, Dex at 2 mg/Kg n=2, Dex at 0.2 mg/Kg n=3, INX201J D-1 n=5, INX201J D-7 n=2, INX231J D-7 n=3, INX234J D-7 n=5, INX240J D-7 n=4 (SEM, one-way ANOVA, D=day).

### Experiment 4

In this experiment contained in FIG. 86, 4 ADCs which were each conjugated with a different GC payload (P) were evaluated. As shown therein, significant decreases in Ag-specific (Ova Tet+CD8 T cell) responses were observed with INX201P, INX231P and INX234P when dosed 1 or 7 days pre-immunization which was comparable to the effects of Dex dosed on day 0 at 2 mg/Kg. As shown therein, only INX240P showed little efficacy. More specifically the data in FIG. 86 shows Ag-specific CD8 T cell numbers from peripheral blood on day 6 post immunization wherein for technical reasons, 2 samples in the PBS, INX231P and INX234P groups were excluded; for all the other groups n=5 (SEM, one-way ANOVA).

### Conclusions

As noted above the data In Experiments 1˜4 show the following:
(i) EXPERIMENT 1 shows that Dex dosed 2h pre-immunization at both 2 and 0.2 mg/Kg efficiently reduces Ag-specific responses;
(ii) EXPERIMENT 2 shows that an exemplary ADC conjugate according to the invention, INX201J, when dosed 24 h, 48h or 96h pre-immunization at 0.2 mg/Kg of GC payload shows similar efficacy as Dex dosed 2h pre-immunization at 2 mg/Kg in reducing Ag-specific responses;
(iii) EXPERIMENT 3 shows that 4 exemplary ADCs showed significant efficacy when dosed 1 or 7 days pre-immunization, at 0.2 mg/Kg of GC payload, in reducing Ag-specific responses. By contrast Dex showed efficacy when dosed at 2 mg/Kg but lost efficacy at 0.2 mg/Kg or when dosed at 2 mg/Kg 7 days pre-immunization; and
(iv) EXPERIMENT 4 shows that 4 exemplary ADCs which were respectively conjugated with a different GC payload. Again, significant decreases in Ag-specific responses were observed for all tested ADCs when dosed 1 or 7 days pre-immunization except for INX240P.

Altogether, these data show that:
(i) exemplary ADCs according to the invention dosed at 0.2 mg/Kg of GC payload have comparable efficacy in decreasing the Ag-specific response as Dex dosed at 2 mg/Kg;
(ii) that both the J and P GC payloads have comparable potency;
(iii) while Dex loses potency if injected 7 days pre-immunization, the different ADCs still have significant potency in controlling the development of an Ag specific responses.

### Example 13: Efficacy of Anti-VISTA Antibody Drug Conjugates in the OVA-Asthma Mouse Model

Asthma is a complex inflammatory disease clinically characterized by airway hyperresponsiveness, inflammatory cell infiltration in bronchoalveolar lavage fluid (BALF) and bronchial walls, and airway structural changes. Inhaled glucocorticoids (GCs) are considered as standard of care for most asthma types. Based thereon studies were conducted to evaluate the therapeutic efficacy of an exemplary antibody drug conjugate (ADC) INX201J, in a mouse model of allergic asthma.

Briefly, as discussed in detail below and shown in the Figures referenced in this example mice were sensitized with 2 injections of ovalbumin (OVA) emulsified in aluminium hydroxide at one week interval. After 1 or 2 weeks (Part 1 and Part 2 of the experiment), mice were challenged with daily exposure via inhalation to OVA for 5 days in a row. Treatment consisted of 3 doses of INX201J at 10 mg/Kg (or 0.2 mg/Kg of payload) or dexamethasone (Dex) at 2 mg/Kg daily during OVA exposure. Analyses were conducted 24h post the last challenge.

These experiments were again conducted in human VISTA knock-in (hVISTA KI) mice which have the human VISTA cDNA knocked-in in place of the mouse VISTA gene, and express human VISTA both at RNA and protein levels with the same expression pattern as mouse VISTA or C57BI/6 mice. The objective of these studies was to evaluate the therapeutic efficacy of our ADC, INX201J, as compared to free dexamethasone (Dex), in the murine model of OVA asthma.

To evaluate the efficacy level of our ADC, we measured by flow cytometry the number of inflammatory cells recruited to the lungs as well as cytokine production in the BAL. Systemic response was evaluated by ELISA to quantify the production of OVA specific IgG and IgE. Finally, we did a blind analysis of H&E stained lung sections to score for disease.

As discussed in detail below these experiments were conducted using 2 different time points for the OVA challenge as we evaluated 2 different protocols described in the literature in parallel which can be considered as an internal repeat.

### Materials and Methods

### Experiment Design

The experiments comprised the following groups with 10 female mice per group. Groups 1-3 and 5,6 are C57BI/6, mice from groups 4 and 7 are human VISTA KI mice. All mice from groups 2 to 7 were sensitized to OVA and challenged with OVA.
Group 1: naive
Group 2: OVA alum-inhalation D14-18
Group 3: OVA alum-inhalation D14-18-Dex at 2 mg/Kg
Group 4: OVA alum-inhalation D14-18-INX201J at 10 mg/Kg
Group 5: OVA alum-inhalation D21-25
Group 6: OVA alum-inhalation D21-25-Dex at 2 mg/Kg
Group 7: OVA alum-inhalation D21-25-INX201J at 10 mg/Kg

Mice from group 2 to 7 were all sensitized with ovalbumin at 10 μg/mouse emulsified in aluminum hydroxide.

### Part 1 of the Experiment:

Five mice from group 1 (naïve) and all the animals from groups 2-4 were subjected to OVA inhalation (3% OVA in PBS) for 30 min for 5 days in a row from day 14 to 18. Dex at 2 mg/Kg was injected i.p. daily from day 14 to 18. INX201J at 10 mg/Kg was dosed i.p. on day 13, 15 and 17. The treated animals were sacrificed on day 19.

### Part 2 of the Experiment:

Five mice from group 1 and all the animals from groups 5-7 were subjected to OVA inhalation (1% OVA in PBS) for 30 min for 5 days in a row from day 21 to 25. Dex at 2 mg/Kg was injected i.p. daily from day 21 to 25. INX201J at 10 mg/Kg was dosed i.p. on day 20, 22 and 24. The treated animals were sacrificed on day 25.

The experiment design and analyses were based on the literature (Gueders et al, “Mouse models of asthma: a comparison between C57BL/6 and BALB/c strains regarding bronchial responsiveness, inflammation, and cytokine production”, Inflamm. Res. (2009) 58:845-854; Yu et al, “Establishment of different experimental asthma models in mice”, Experimental and Therapeutic Medicine 15:2492-2498, 2018).

### Test Agents and Dosage

### Antibodies

INX201J (Abzena, Lot #s: JZ-0556-025-1, JZ-0556-027, JZ-0556-013). INX201 is a humanized anti-human VISTA antibody on a human IgG1/kappa backbone with L234A/L235A/E269R/K322A silencing mutations in the Fc region. INX201J is the conjugated antibody with a drug/antibody ratio of 8.0, conjugated via full modification of the interchain disulfides. The linker/payload (J) consists of a protease sensitive linker with a budesonide analog payload. INX201J was diluted in PBS and injected intraperitoneal (i.p.) in a volume of 0.2 ml to deliver a specified dose.

### Dexamethasone

Dexamethasone sterile injection from Phoenix, NDC 57319-519-05, was diluted in PBS and dosed as described via i.p. injection.

### Ovalbumin

Ovalbumin (or albumin from chicken egg whites) was purchased from Sigma (A5503) and resuspended in PBS. It was dosed i.p. or via nebulizer.

### Mice

The hVISTA KI mice were bred on site (Center for Comparative Medicine and Research at Dartmouth). All the experiments were done in female mice enrolled at 15 weeks of age. C57BI/6 mice were purchased from Jackson Laboratories.

### OVA Inhalation

OVA was delivered via nebulizer using the nebulizer delivery system from Kent Scientific (AG-ALSM-0530LG).

### Bleed

Peripheral blood was harvested from the retro-orbital cavity using a glass Pasteur pipette that was first rinsed with heparin to prevent coagulation. Blood was then centrifuged at 550 rcf for 5 min and 75 μl of plasma collected and stored at −80° C. before cytokine analysis. Blood cells were resuspended with 75 μl of PBS and processed for immunostaining.

### Bronchoalveolar Lavage

Mice were sacrificed by CO2 inhalation, and a bronchoalveolar lavage was immediately performed using 5×1 ml PBS-EDTA (0.5 mM). Cells were recovered by gentle manual aspiration. Volumes were recorded. Samples with a recovery volume below 4 ml were excluded. After centrifugation at 550 rcf for 5 min, supernatant was collected and frozen at −80° C. for protein assessment. Cells were resuspended in PBS and processed for immunostaining.

### BAL Immunostaining

BAL cell samples divided in 2 and stained with 2 different antibody panels for lymphocytes and for myeloid cells (See TABLE 2 and TABLE 3). After 30 min at 4˜C, samples were washed once and resuspended in a fixed volume. The fixed volume was analyzed on a MacsQuant flow cytometer to obtain comparable cell numbers.

TABLE 2
Lymphocyte panel

| Antibody | Dilution |
|---|---|
| CD3 FITC | 200 |
| B220 PE | 200 |
| CD45 PerCP | 400 |
| CD4-PECy7 | 200 |
| VISTA APC | 200 |
| CD8 APC-cy7 | 200 |
| FoxP3 BV421 | 200 |
| Live/Dead dye | 1000 |
| mFc block | 200 |

TABLE 3
Myeloid panel

| Antibody | Dilution |
|---|---|
| Ly6G FITC | 200 |
| Siglec F PE | 200 |
| CD45 PerCP | 400 |
| CD193-PECy7 | 200 |
| VISTA APC | 200 |
| F4/80 APC-cy7 | 200 |
| CD11b BV421 | 200 |
| Live/Dead dye | 1000 |
| mFc block | 200 |

### Whole Blood Immunostaining

We used the 1-wash protocol that allows for absolute blood cell count. 10 μl of antibody cocktail (See below) was directly added to 100 μl of blood. After 30 min incubation at room temperature (RT), 600 μl BD FACS lysis buffer was added to the sample. After 30 min incubation at RT, samples were spun at 550 rcf for 5 min, wash once in PBS, resuspended in a fixed volume of PBS. The whole sample was run on a MacsQuant flow cytometer to obtain an absolute cell number.

As shown in TABLE 4 and TABLE 5 different antibody panels were used for the lymphocytes and myeloid cells.

TABLE 4
Lymphocyte panel

| Antibody | Dilution |
|---|---|
| CD3 FITC | 200 |
| B220 PE | 200 |
| CD45 PerCP | 400 |
| CD4-PECy7 | 200 |
| VISTA APC | 200 |
| CD8 APC-cy7 | 200 |
| FoxP3 BV421 | 200 |
| mFc block | 200 |

TABLE 5
Myeloid panel

| Antibody | Dilution |
|---|---|
| Ly6G FITC | 200 |
| Siglec F PE | 200 |
| CD45 PerCP | 400 |
| Ly6G-PECy7 | 200 |
| VISTA APC | 200 |
| F4/80 APC-cy7 | 200 |
| Cd11b BV421 | 200 |
| mFc block | 200 |

### ELISA

### ELISA for IgG, OVA-Specific IgG, IgE, OVA-Specific IgE

### ELISA for Mouse IgG1

First, 96-well flat-bottom plates (Thermo Scientific Nunc Immunol Maxisorp, cat #442404) were coated with goat anti-mouse IgG1 (Southern Biotech, cat #1070-01) at 1 μg/ml in PBS for one hour at RT. The wells were washed 3 times with PT (PBS with 0.05% Tween 20) then blocked with PTB (PBS with 0.05% Tween 20 and 1% BSA) for one hour at RT. Mouse IgG1 anti-ovalbumin (Biolegend, cat #520502) was used to build a standard curve. The wells were washed 3 times with PT then plasma samples were incubated at up to 4 different dilutions in PTB (to fit on the standard curve) for 1 hour at RT.

After 3 washes with PT, goat anti-mouse IgG1-HRP (Southern Biotech, cat #1070-05) was used at 1/20,000 as a detection reagent, incubating 1 hour at RT. Following 3 washes, the ELISA reaction was revealed using TMB substrate following manufacturer instructions. After 5-10 min at RT, the reaction was stopped with 1M H₂SO₄.

### ELISA for Mouse IgG1 Anti-Ovalbumin

First, 96-well flat-bottom plates (Thermo Scientific Nunc Immunol Maxisorp, cat #442404) were coated with ovalbumin (Sigma, cat #1070-01) at 95 μg/ml in PBS for one hour at RT. The wells were washed 3 times with PT then blocked with PTB for one hour at RT. Mouse IgG1 anti-ovalbumin (Biolegend, cat #520502) was used to build a standard curve. The wells were washed 3 times with PT then plasma samples were incubated at up to 4 different dilutions in PTB (to fit on the standard curve) for 1 hour at RT.

After 3 washes with PT, goat anti-mouse IgG1-HRP (Southern Biotech, cat #1070-05) was used at 1/20,000 as a detection reagent, incubating 1 hour at RT. Following 3 washes, the ELISA reaction was revealed using TMB substrate following manufacturer instructions. After 5-10 min at RT, the reaction was again stopped with 1M H₂SO₄.

### ELISA for Mouse IgE

First, 96-well flat-bottom plates (Thermo Scientific Nunc Immunol Maxisorp, cat #442404) were coated with goat anti-mouse IgE (Southern Biotech, cat #1110-01) at 1 μg/ml in PBS for one hour at RT. The wells were washed 3 times with PT then blocked with PTB for one hour at RT. Mouse IgE anti-ovalbumin (BioRad, cat #MCA2259) was used to build a standard curve. The wells were washed 3 times with PT then plasma samples were incubated at up to 4 different dilutions in PTB (to fit on the standard curve) for 1 hour at RT.

After 3 washes with PT, goat anti-mouse IgE-HRP (Southern Biotech, cat #1110-05) was used at 1/2000 as a detection reagent, incubating 1 hour at RT. Following 3 washes, the ELISA reaction was revealed using TMB substrate following manufacturer instructions. After 5-10 min at RT, the reaction was stopped with 1M H₂SO₄.

### ELISA for Mouse IgE Anti-Ovalbumin

First, 96-well flat-bottom plates (Thermo Scientific Nunc Immunol Maxisorp, cat #442404) were coated with ovalbumin (Sigma, cat #1070-01) at 95 μg/ml in PBS for one hour at RT. The wells were washed 3 times with PT then blocked with PTB for one hour at RT. Mouse IgG1 anti-ovalbumin (BioRad, cat #MCA2259) was used to build a standard curve. The wells were washed 3 times with PT then plasma samples were incubated at up to 4 different dilutions in PTB (to fit on the standard curve) for 1 hour at RT.

After 3 washes with PT, goat anti-mouse IgE-HRP (Southern Biotech, cat #1110-05) was used at 1/2000 as a detection reagent, incubating 1 hour at RT. Following 3 washes, the ELISA reaction was revealed using TMB substrate following manufacturer instructions. After 5-10 min at RT, the reaction was stopped with 1M H₂SO₄.

### ELISA for Cytokines

R&D (cat #DY420-05) Duoset mouse CCL11/Eotaxin.
R&D (cat #DY478-05) Duoset mouse CCL5/RANTES
R&D (cat #DY405-05) Duoset mouse IL-5.
R&D (cat #DY413-05) Duoset mouse IL-13

All ELISAs were conducted following the manufacturer's included protocol.

### Histopathological Lung Scoring

Lung were dissected, formalin fixed and processed for paraffin embedding. Disease scoring was conducted in a blind manner on H&E stained sections, and scores assigned as follows:
4: infiltrate abundant and spread all over-loss of lung structure
3: infiltrate abundant and spread all over-limited damage to lung structure
2: infiltrate visible as large foci
1: infiltrate visible as small foci
0: normal

### Results

### Blood Responses

### Cellular Responses

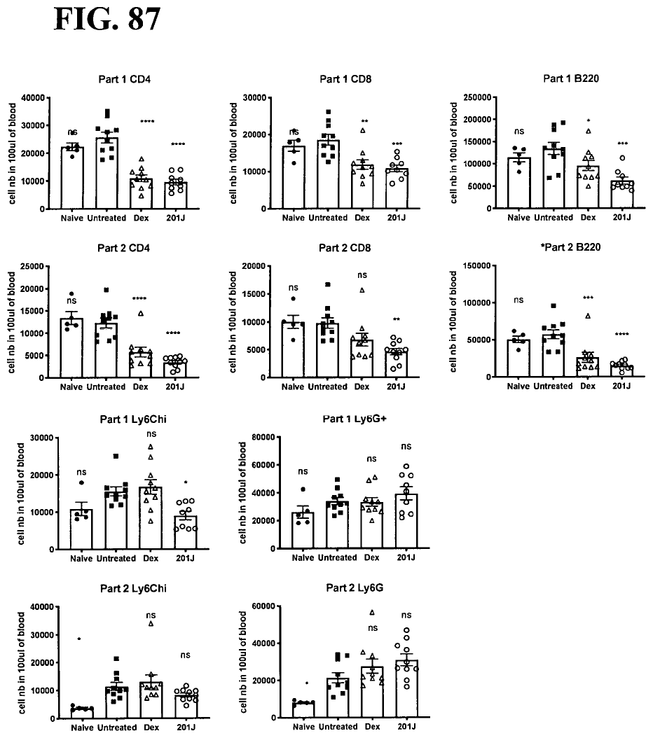

As shown in the experiment in FIG. 87 no disease-driven changes in lymphocytes or myeloid cells were observed in the peripheral circulation with the 2 experiment schedules, and GC treatment led to similar decreases in B and T cells when administered free (Dex) or conjugated (INX201J). Specifically, FIG. 87 shows changes in absolute cell numbers in peripheral blood in the 2 experiment schedules. OVA challenge on days 14 to 18 (Part 1) and on days 21 to 25 (Part 2) (SEM, one-way ANOVA, n=10 except for naïve group with n=5).

### Immunoglobulin Responses

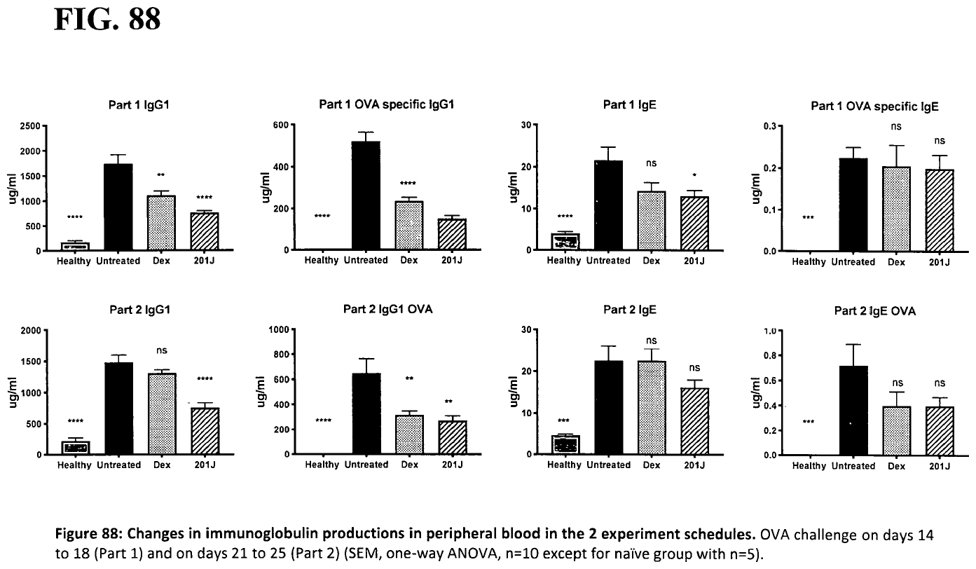

As shown in the experiment in FIG. 88, untreated OVA challenged animals showed dramatic increases in IgG1 and IgE as well as OVA specific Ig as compared to naïve animals. Both Dex and INX201J treated groups showed similar significant decreases in IgG1, IgG1 OVA specific production. Limited to no decreases were observed with IgE and OVA specific IgE. Particularly, FIG. 88 shows changes in immunoglobulin productions in peripheral blood in the 2 experiment schedules. OVA challenge on days 14 to 18 (Part 1) and on days 21 to 25 (Part 2) (SEM, one-way ANOVA, n=10 except for naïve group with n=5).

### Responses in Bronchoalveolar Lavage

### Cellular Responses

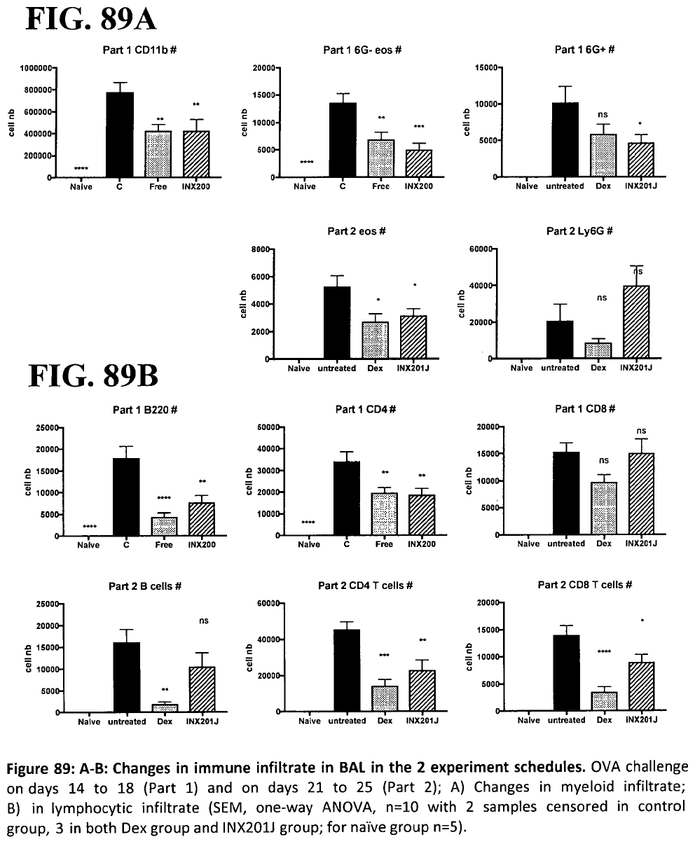

As shown in the experiment in FIG. 89, OVA challenges caused the recruitment of large inflammatory infiltrates in the bronchoalveolar space composed of both lymphocytes and myeloid cells. INX201J treatment led to similar decreases in immune infiltrates when compared to Dex in both experiment schedules except for CD8 T cells. Notably, INX201J showed the same potency as Dex in decreasing eosinophil numbers (defined as CD11b+, Ly6G−, SiglecF+CD193+). Particularly, FIG. 89 shows changes in immune infiltrate in BAL in the 2 experiment schedules. OVA challenge on days 14 to 18 (Part 1) and on days 21 to 25 (Part 2); A) Changes in myeloid infiltrate; B) in lymphocytic infiltrate (SEM, one-way ANOVA, n=10 with 2 samples censored in control group, 3 in both Dex group and INX201J group; for naïve group n=5).

### Cytokine Changes

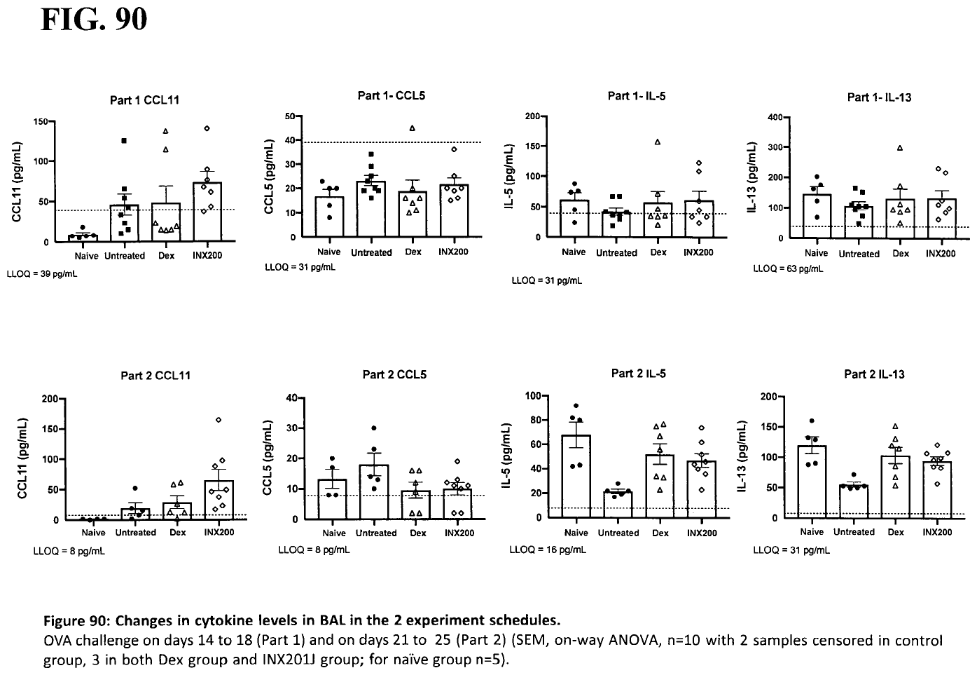

The experiment in FIG. 90 indicates that except for CCL11 that showed limited increases following disease induction, all the other cytokines evaluated showed no changes. Neither INX201J nor Dex treatments had any impact on cytokine levels in BAL. Specifically FIG. 90 shows the changes in cytokine levels in BAL in the 2 experiment schedules. OVA challenge on days 14 to 18 (Experiment Part 1) and on days 21 to 25 (Experiment Part 2) (SEM, on-way ANOVA, n=10 with 2 samples censored in control group, 3 in both Dex group and INX201J group; for naïve group n=5).

### Lung Disease Score

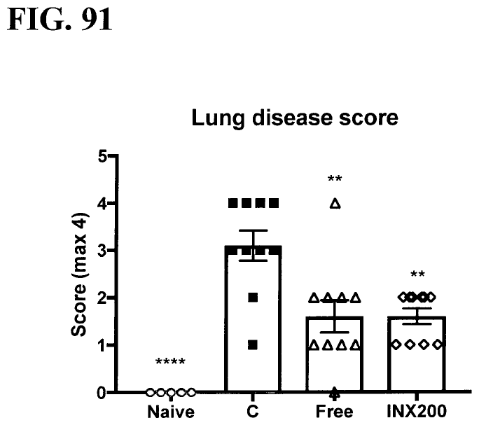

As shown in the experiment in FIG. 91 (Experiment Part 1, SEM, one-way ANOVA, n=10 except for naïve group n=5); significant damages were observed in the untreated lungs including loss of bronchoalveolar morphology and massive recruitment of inflammatory cells. It can be seen from these results that both INX201J and Dex treatment similarly and significantly reduced lung damage with limited structural damage and inflammatory infiltrates.

### Conclusions

The experiments in FIGS. 87-91 provide specific evidence that INX201J treatment has equivalent impact as free Dex (dosed >10-fold higher) in the following:
reducing the recruitment of inflammatory infiltrate in the bronchoalveolar lavage (BAL)
reducing damage at the histopathological level of the lungs
reducing the production of IgG1 and more specifically anti-OVA IgG1 in blood circulation
eliciting no changes in IgE or anti-OVA IgE was observed with both therapeutic approaches in blood circulation
eliciting no changes in cytokine production was observed with both therapeutic approaches in the bronchoalveolar lavage

Of significance, similar results were observed in the 2 parts/2 different schedules of these experiments.

### Example 14: Impact of Exemplary Anti-VISTA Antibody Drug Conjugates on VISTA Expressing Immune Cells

In these experiments we evaluated the targeting specificity of the antibody drug conjugate (ADC) INX231J, an anti-human VISTA monoclonal antibody linked to a glucocorticoid (GC) payload. To monitor/confirm GC delivery and activity, we measured the transcriptional activation of FKBP5 by quantitative Real Time PCR (qRT-PCR) (1). These experiments were again conducted in human VISTA knock-in (hVISTA KI) mice which have the human VISTA cDNA knocked-in in place of the mouse VISTA gene, and express human VISTA both at RNA and protein levels with the same expression pattern as mouse VISTA.

Particularly, we evaluated the impact of non-specific ADC internalization by two different approaches. First, we added a human IgG1 silent conjugated to the same payload; second, we ran the same experiment in C57BI/6 mice that do not express the human VISTA target (mouse VISTA only). Briefly, INX231J or INX231P, human IgG1siJ, or free dexamethasone (Dex) were delivered in vivo via intraperitoneal (i.p.) injection. After 20h for INX231J/hlgG1siJ/INX231P and 2h for Dex, blood cells and splenocytes were isolated, RNA extracted and FKBP5 transcriptional levels evaluated.

The objective of these experiments was to validate the targeting specificity of our ADC to human VISTA expressing cells/tissues as compared to free dexamethasone (Dex). To monitor/confirm GC delivery and activity, we measured by quantitative Real Time PCR (qRT-PCR) the transcriptional activation of FKBP5, a sensitive and early GC response gene. We have previously shown in this application that Dex treatment causes dramatic increases in FKBP5 messenger RNA in VISTA expressing cells 2-4h post treatment, but that the transcriptional impact is gone by 24h. In contrast, the ADC's impact on FKBP5 transcription is long-lasting, with peak induction at 20h post treatment but signal is still detectable for 3 days in monocytes and 14 days in macrophages.

In these experiments, we used the anti-VISTA antibody INX231 with 2 different payloads (J and P) or free Dex delivered in vivo via intravenous (i.v.) or intraperitoneal (i.p.) injections respectively. Splenocytes and blood cells were isolated, RNA was extracted and FKBP5 transcriptional levels evaluated. These experiments and the results thereof are described in detail below.

### Materials and Methods

### Experiment Design

For all 3 studies:

Dex was injected i.p. at 2h before mouse euthanasia and cell isolation, which corresponds to peak FKBP5 induction.

The ADC (INX231J or INX231P or hlgG1siJ) was injected i.v. 20h before mouse euthanasia and cell isolation, to provide sufficient time for ADC processing and peak FKBP5 induction. To note, ADCs were injected i.v. to ensure more consistent delivery of large molecules.

A control group injected with PBS only was included to define FKBP5 transcript baseline.

### Test Agents and Dosage

### Antibodies

INX231 (lot #72928.1.a) is a humanized anti-human VISTA antibody on a human IgG1/kappa backbone with L234A/L235A/E269R/K322A silencing mutations in the Fc region.
INX231J (lot #JZ-0556-013-1) is INX231 with a drug/antibody ratio (DAR) of 8.0, conjugated via full modification of the interchain disulfides. The linker/payload (J) consists of a protease sensitive linker with a budesonide analog payload.
INX231P (lot #JZ-0556-017-1) is INX231 with a drug/antibody ratio (DAR) of 8.0, conjugated via full modification of the interchain disulfides. The linker/payload (P) consists of a protease sensitive linker with a budesonide analog payload.
Human IgG1siJ (lot #JZ-0556-025-2) is an anti-RSV mAb on a human IgG1/kappa backbone with E269R/K322A silencing mutations in the Fc region. The drug/antibody ratio is 8.0, conjugated via full modification of the interchain disulfides with the J linker/payload.

Five mice from group 1 and all the animals from groups 5-7 were subjected to OVA inhalation (1% OVA in PBS) for 30 min for 5 days in a row from day 21 to 25. Dex at 2 mg/Kg was injected i.p. daily from day 21 to 25. INX201J at 10 mg/Kg was dosed i.p. on day 20, 22 and 24. The treated animals were sacrificed on day 25. All ADC were diluted in PBS and injected i.v. in a final volume of 0.2 ml to deliver a specified dose.

### Dexamethasone

Dexamethasone sterile injection solution from Phoenix, NDC 57319-519-05, was diluted in PBS and dosed as described via i.p. injection.

### Mice

The hVISTA KI mice were bred on site (Center for Comparative Medicine and Research at Dartmouth); C57BI/6 mice were received from Jackson Laboratories (ref #000665).

Male or female mice were enrolled between 9 and 15 weeks of age.

### Cell Isolation

After euthanasia, cardiac blood (volume ranging between 0.3 and 0.5 ml) and spleen were collected.

Blood prep: 6 ml of ACK buffer was added to the blood for red blood cell lysis. After 5 min at RT, cells were spun down at 1500 rpm for 5 min; after one wash in 10 ml of PBS, cells were pelleted and directly resuspended in RNA lysis buffer.

Spleens were dissociated mechanically. After passage through a 40 μm filter, cell pellets were resuspended in RNA lysis buffer (See below).

### RNA Preparation and Real Time PCR

Cell pellets from blood and spleen were resuspended in 0.4 ml of RNA lysis buffer from NucleoSpin® RNA Plus kit (Macherey-Nagel #740984). RNA was isolated following manufacturer's instructions and eluted in 30 or 40 ml H₂O (RNase/DNase free). RNA concentration was assessed on Nanodrop.

Reverse transcription was done using Taqman reverse transcription reagents (#N8080234) and following manufacturer's instructions.

Quantitative Real-Time PCR was done using Taqman master mix 2× kit (#4369016) and Taqman primers for mouse FKBP5 (Mm00487401_m1), and mouse HPRT as housekeeping gene (Mm446968_m1) and run on a QuantStudio3 from Applied Biosystem.

Ct data were converted to ΔCt and A4Ct or Log 2 fold changes to PBS.

### Experiment 1

In this experiment, we evaluated the impact of human IgG1 silent control conjugated to the J payload (IgG1siJ) vs. INX231J and Dex on VISTA expressing tissues (blood and spleen) in hVISTA KI male mice. IgG1siJ and INX231J were dosed at 5 mg/Kg (delivering 0.1 mg/Kg of payload) and FKBP5 induction was measured 20h later, providing sufficient time for ADC processing and robust FKBP5 induction. Dex was injected at 2 mg/Kg and FKBP5 induction measured 2h later.

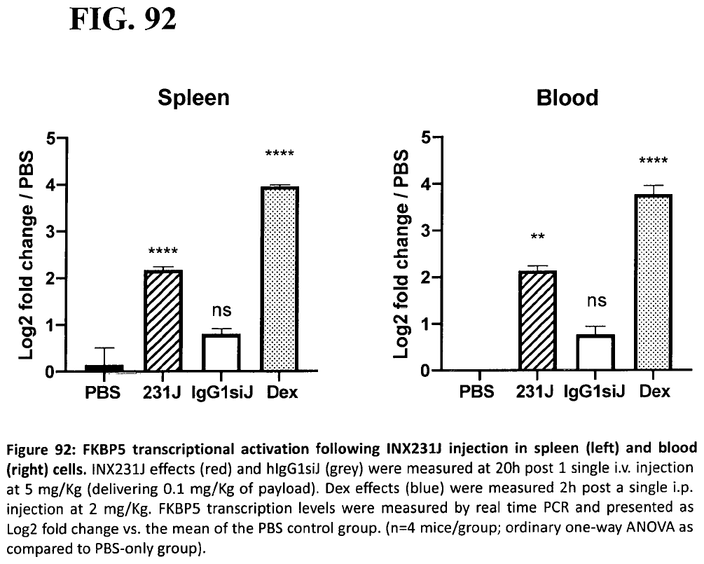

As shown in the experiment in FIG. 92, while robust FKBP5 inductions were observed with INX231J and Dex treatment, the conjugated Ig control caused only small, non-significant changes in FKBP5 signal. More specifically, FIG. 92 shows FKBP5 transcriptional activation following INX231J injection in spleen (left) and blood (right) cells. INX231J effects and hlgG1siJ were measured at 20h post 1 single i.v. injection at 5 mg/Kg (delivering 0.1 mg/Kg of payload). Dex effects were measured 2h post a single i.p. injection at 2 mg/Kg. FKBP5 transcription levels were measured by real time PCR and presented as Log 2 fold change vs. the mean of the PBS control group. (n=4 mice/group; ordinary one-way ANOVA as compared to PBS-only group).

### Experiment 2

In this experiment, we evaluated the impact of INX231P vs. Dex in C57BI/6 male mice that do not express human VISTA, on blood and spleen cells. INX231P was dosed at 10 mg/Kg (delivering 0.2 mg/Kg of payload) and FKBP5 induction was measured 20h later, providing sufficient time for ADC processing and robust FKBP5 induction. Dex was injected at 2 mg/Kg and FKBP5 induction measured 2h later.

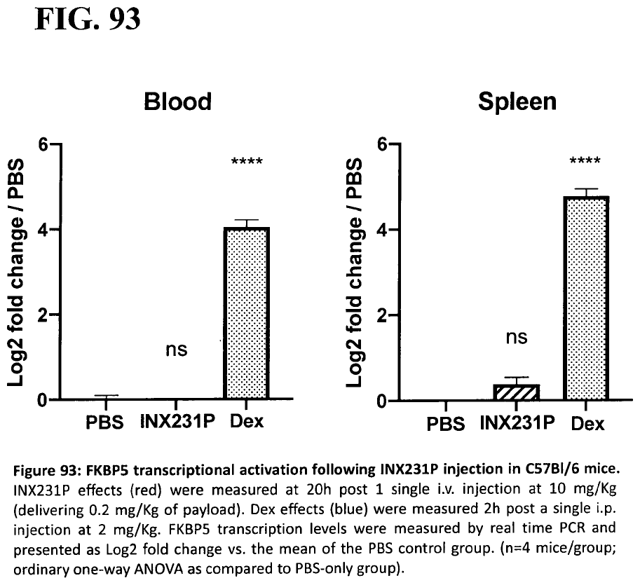

As shown in the experiment in FIG. 93, while robust and significant FKBP5 inductions were observed with Dex treatment, INX231P had no effect on blood and spleen cells of wild type mice, demonstrating that ADC impact is target-driven. More specifically, FIG. 93 shows FKBP5 transcriptional activation following INX231P injection in C57BI/6 mice. INX231P effects were measured at 20h post 1 single i.v. injection at 10 mg/Kg (delivering 0.2 mg/Kg of payload). Dex effects were measured 2h post a single i.p. injection at 2 mg/Kg. FKBP5 transcription levels were measured by real time PCR and presented as Log 2 fold change vs. the mean of the PBS control group. (n=4 mice/group; ordinary one-way ANOVA as compared to PBS-only group).

### Experiment 3

In this experiment, we evaluated the impact of INX231P vs. Dex in C57BI/6 female mice that do not express human VISTA, on blood and spleen cells. We added a hVISTA KI group as control for ADC activity. INX231P was dosed at 10 mg/Kg (delivering 0.2 mg/Kg of payload) and FKBP5 induction was measured 20h later, providing sufficient time for ADC processing and robust FKBP5 induction. Dex was injected at 2 mg/Kg and FKBP5 induction measured 2h later.

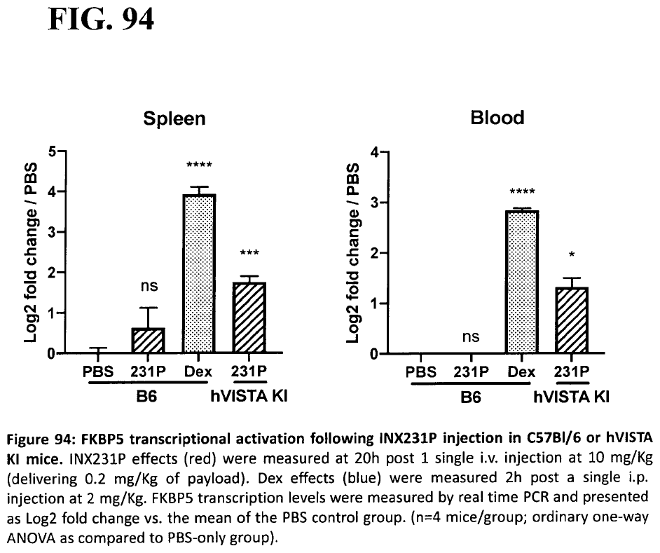

As shown in the experiment in FIG. 94, while robust and significant FKBP5 induction is observed with Dex treatment, INX231P had a non-significant effect on blood and spleen cells of wild type mice, demonstrating that the ADC impact is target-driven. In contrast, INX231P treatment at same dosage in hVISTA KI animals led to strong and significant induction of FKBP5 transcript demonstrating the ADC potency on its target population. More specifically, FIG. 84 shows FKBP5 transcriptional activation following INX231P injection in C57BI/6 or hVISTA KI mice. INX231P effects were measured at 20h post 1 single i.v. injection at 10 mg/Kg (delivering 0.2 mg/Kg of payload). Dex effects were measured 2h post a single i.p. injection at 2 mg/Kg. FKBP5 transcription levels were measured by real time PCR and presented as Log 2 fold change vs. the mean of the PBS control group. (n=4 mice/group; ordinary one-way ANOVA as compared to PBS-only group).

### Conclusions

The results of EXPERIMENT 1 shows that in hVISTA KI, while INX231J and Dex induced robust levels of FKBP5 in spleen and blood cells, the human IgG1 silent steroid conjugated control had little to no impact on FKBP5 transcription levels in both tissues.

The results of EXPERIMENT 2 shows that in male C57BI/6 mice, in the absence of the human VISTA target, INX231P has no impact on FKBP5 transcription levels in VISTA-expressing blood cells or splenocyte, while free steroid induced robust levels of FKBP5 in both tissues.

The results of EXPERIMENT 3 which is a repeat of EXPERIMENT 2 in female C57BI/6 mice, with the addition of a positive control of hVISTA KI mice, show that in the absence of the human VISTA target, INX231P has little to no impact on FKBP5 transcription levels in VISTA-expressing blood cells or splenocytes. In contrast, INX231P at same dosing in hVISTA KI mice or Dex induced robust levels of FKBP5 in both tissues.

Altogether, the data demonstrate that the presence of the human VISTA target is necessary for efficient cellular delivery of GC by the ADC, regardless of the GC payload.

### Example 15: Impact of Exemplary Anti-VISTA Antibody Drug Conjugates on Ex Vivo Monocyte Activation (Acute (One Day)) Assessment

The experiments in this example were conducted to evaluate the efficacy and pharmacodynamic range of the antibody drug conjugate (ADC) INX231P, an anti-human VISTA monoclonal antibody linked to a glucocorticoid (GC) payload, in monocytes. We showed in an earlier example that the transcription of the GC target gene FKBP5 is upregulated in monocytes up to 3 days post treatment while free Dexamethasone (Dex) impact on FKBP5 is undetectable at 24h.

We further developed a model that allows us to evaluate potential long-term anti-inflammatory impact of ADC on monocytes. Briefly, ADCs were delivered in vivo via intravenous (i.v.) injection, and after 1 to 7 days splenic monocytes were isolated and put in culture. Cells were then activated with different concentrations of lipopolysaccharide (LPS), causing dramatic increases in cytokine production at 24h. Dex treatment 2h before monocyte isolation robustly reduces cytokine production.

Three experiments (EXPERIMENTS 1, 2 and 3 discussed below) were conducted in human VISTA knock-in (hVISTA KI) mice which have the human VISTA cDNA knocked-in in place of the mouse VISTA gene, and express human VISTA both at RNA and protein levels with the same expression pattern as mouse VISTA. The objective of these studies was to evaluate the impact of INX231P in vivo treatment specifically on monocytes, which express high levels of VISTA. A second objective was to compare its anti-inflammatory capabilities to its agonist counterpart INX901. Briefly, ADCs were delivered in vivo via intravenous (i.v.) injection, and after 1 to 7 days spleen monocytes were isolated and put in culture. Cells were then activated with different concentration of LPS and supernatants were collected at 24h to evaluate cytokine response (by Luminex mouse 32-plex (EXPERIMENT 1) or ELISA for selected cytokines (EXPERIMENT 2 and EXPERIMENT 3)).

### Materials and Methods

For all 3 experiments Dex was injected i.p. at 2h before mouse euthanasia and cell isolation, at optimal response. In EXPERIMENT 2 and EXPERIMENT 3, both the ADC INX231P and the agonist counterpart INX901 were injected i.v. 24h before mouse euthanasia and cell isolation, to provide sufficient time for ADC processing. Also, a control group injected with PBS was included to define maximal cytokine responses.

### Test Agents and Dosage

### Antibodies

INX231 (lot #72928.1.a) is a humanized anti-human VISTA antibody on a human IgG1/kappa backbone with L234A/L235A/E269R/K322A silencing mutations in the Fc region.
INX231P (lot #JZ-0556-017-1) is INX231 with a drug/antibody ratio (DAR) of 8.0, conjugated via full modification of the interchain disulfides. The linker/payload (P) consists of a protease sensitive linker with a budesonide analog payload.
INX901 (Lot #BP-021-016-23) is a humanized anti-human VISTA antibody on a human IgG2/kappa backbone.

All antibodies and ADC were diluted in PBS and injected intravenous (i.v.) in a volume of 0.2 ml to deliver a specified dose.

### Dexamethasone

Dexamethasone sterile injection solution from Phoenix, NDC 57319-519-05, was diluted in PBS in a volume of 0.2 ml and dosed as described via intraperitoneal (i.p.) injection.

### Mice

The hVISTA KI mice were bred on site (Center for Comparative Medicine and Research at Dartmouth); C57BI/6 mice were received from Jackson Laboratories (ref #000665). Male or female mice were enrolled between 9 and 15 weeks of age.

### Spleen Monocyte Isolation

In EXPERIMENT 1 and EXPERIMENT 2, cells were isolated using the EasySep™ Mouse Monocyte Isolation Kit from StemCell (Catalog #19861) following manufacturer's instructions; in ADC-INVIVO-109, the Monocyte Isolation Kit from Miltenyi was used (catalog #130-100-629). Similar cell number and purity were obtained across experiments.

### Ex Vivo LPS Stimulation Assay

After counting, cells were plated at ˜100,000 cells/well depending on the number of cells isolated (Note that all reported data were normalized to the plated cell number) and as singlicates. LPS was added to tissue culture medium at 0, 10 or 100 ng/ml as described. Cell supernatants were collected at 24h for cytokine analysis.

### Cytokine Analyses

### Experiment 1

Cytokine analyses were conducted on 25 μl of supernatant using a Millipore mouse 32-plex platform; the Immune Monitoring Lab (IML, Shared Resources at Dartmouth-Hitchcock Norris Cotton Cancer Center) performed the analyses. See following website for all protocol and analysis descriptions http://www.dartmouth.edu/˜dartlab/?page=multiplexed-cytokines.

### Experiments 2 and 3

Cytokine analyses were conducted via ELISA for TNFα, MIP-1a and MIP-1b using the following kits:
Mouse CCL3/MIP-1alpha DuoSet ELISA (R&D #DY450-05)
Mouse CCL3/MIP-1beta DuoSet ELISA (R&D #DY451-05)
Mouse TNFα ELISA (Biolegend cat #430904)

All the ELISA were conducted following manufacturers' instructions.

### Results

### Experiment 1: Impact of Dexamethasone on Ex Vivo LPS Stimulation of Monocytes Isolated from Spleen

In EXPERIMENT 1, we evaluated the impact of in vivo treatment with Dex at 2 different doses on spleen monocytes ex vivo. Briefly, female C57BI/6 mice were treated with Dex at 2 or 0.2 mg/Kg injected i.p. The control group received PBS. After 2 h, animals were sacrificed and the spleens were collected. Monocytes were isolated and put in culture. Because of low monocyte number post isolation, the 5 samples per group were pooled into 2 samples for plating (pool of 2 or 3 initial samples). The cytokine data were then normalized to cell number afterward.

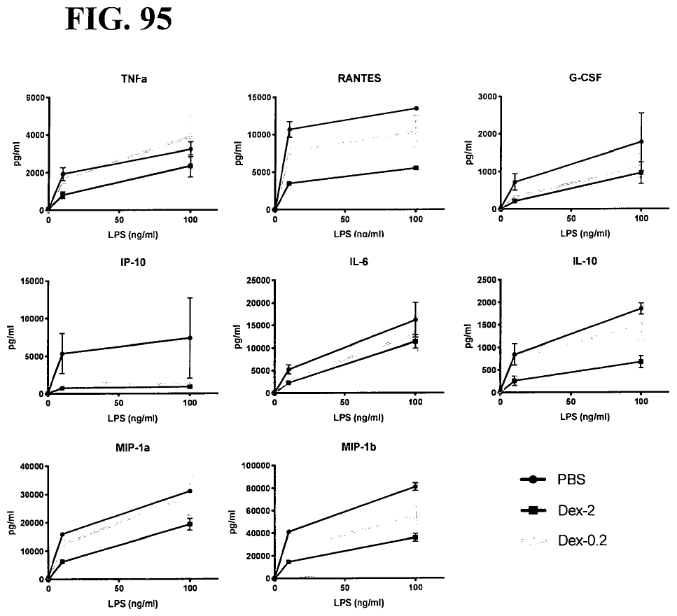

After plating, cells were treated with LPS at 10 or 100 ng/ml or untreated. Cell supernatants were collected at 30 min and 24h. Cytokine production was analyzed on a mouse 32-plex. No changes in cytokine levels were observed at 30 min (not shown). At 24 h, 8 cytokines G-CSF, IL-6, IL-10, IP-10, MIP-1a, MIP-1b, TNFα and RANTES were upregulated by LPS treatment in spleen samples. As shown in FIG. 95 Dex in vivo treatment led to reduced cytokine responses at both LPS concentrations. More specifically, FIG. 95 shows that in vivo Dex treatment elicits a substantial decrease in ex vivo monocyte inflammatory response to LPS. In the experiment, the mice were injected i.p. with PBS or Dex at 2 mg/Kg or 0.2 mg/Kg. After 2h, spleen monocytes were isolated, put in culture and subjected to LPS stimulation at 0, 10 and 100 ng/ml. 24h supernatants were analyzed on Luminex 32-plex (n=5 mice/group but samples 1,2,3 and 4,5 were pooled into 2 samples).

### Experiment 2: Impact of Dexamethasone Vs INX231P Vs INX901 on Ex Vivo LPS Stimulation of Monocytes Isolated from Spleen

In EXPERIMENT 2, we evaluated the impact of INX231P vs. INX901 (same CDRs as INX231 but on a human IgG2 backbone) vs. Dex in vivo treatment on spleen monocytes from hVISTA KI female mice stimulated ex vivo by LPS. To evaluate the pharmacodynamic range of these molecules, spleen monocytes were isolated 24 h, 3 days and 7 days later for both INX231P and INX901 treated groups and at 2 h, 2 days and 6 days post treatment for the Dex treated group. INX231P and INX901 were dosed at 10 mg/Kg, Dex was injected at 2 mg/Kg. After plating, samples were treated with LPS at 10 ng/ml or untreated. Cell supernatants were collected at 24h. Cytokine analysis was conducted via ELISA for NFa, MIP-1b and MIP-1a. Cytokine data were normalized to plated cell number.

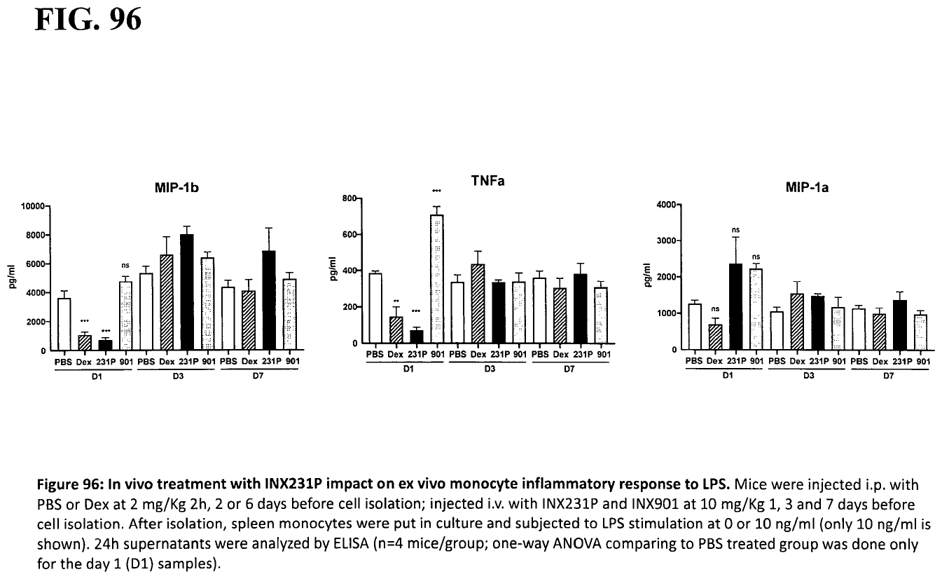

As shown in the experiment in FIG. 96, on day 1 INX231P had robust impact on TNFα and MIP-1b production, comparable to Dex at 2h. No effect was observed at later time points. By contrast, INX901 had no impact (MIP-1a and b) or increased (TNFα) the cytokines analyzed. More particularly, FIG. 96 shows the effects of in vivo treatment with INX231P impact on ex vivo monocyte inflammatory response to LPS. Mice were injected i.p. with PBS or Dex at 2 mg/Kg 2 h, 2 or 6 days before cell isolation; injected i.v. with INX231P and INX901 at 10 mg/Kg 1, 3 and 7 days before cell isolation. After isolation, spleen monocytes were put in culture and subjected to LPS stimulation at 0 or 10 ng/ml (only 10 ng/ml is shown). 24h supernatants were analyzed by ELISA (n=4 mice/group; one-way ANOVA comparing to PBS treated group was done only for the day 1 (D1) samples).

### Experiment 3: Impact of Dexamethasone Vs INX231P Vs INX901 on Ex Vivo LPS Stimulation of Monocytes Isolated from Spleen

In EXPERIMENT 3, we evaluated cytokine response only after 2h for Dex (at 2 mg/Kg) or 24h for antibody treatment (10 mg/Kg). Spleen monocytes were isolated, placed in culture and treated with LPS at 10 or 100 ng/ml. Cell supernatants were collected at 24h. Cytokine analysis was conducted via ELISA for TNFα, MIP-1b and MIP-1a and the data was normalized to plated cell number.

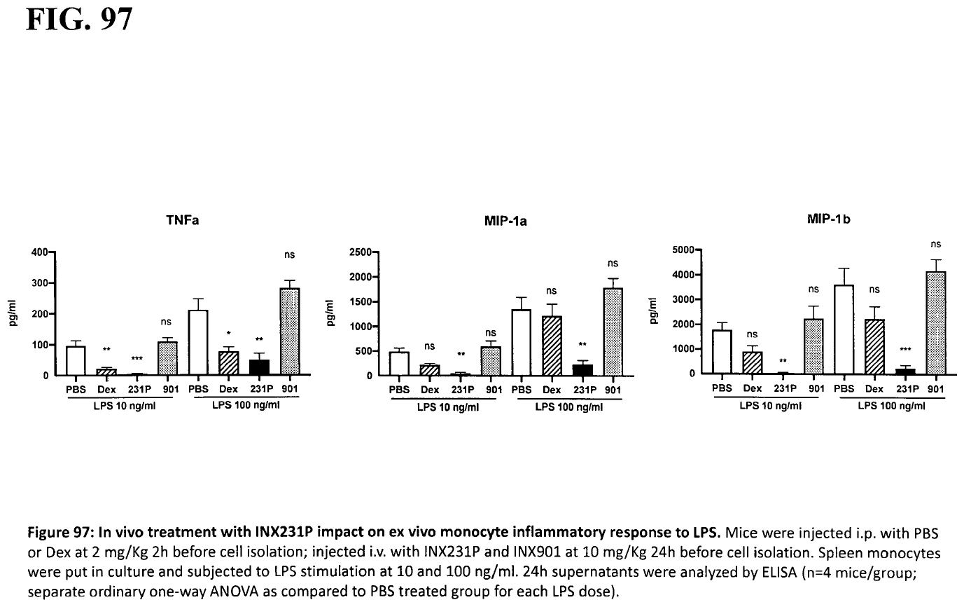

The experiment in FIG. 97 shows that INX231P potently prevented ex vivo activation of monocytes for all 3 cytokines analyzed at both LPS concentrations. To note when cells are stimulated with LPS at 100 ng/ml, Dex treatment appeared to lose potency suggesting that INX231P, while delivering 10 times less payload, is more potent. Finally, as observed in EXPERIMENT 3, INX901 treatment had no effect on LPS induced cytokine responses. More particularly, FIG. 97 shows in vivo treatment with INX231P impact on ex vivo monocyte inflammatory response to LPS. Mice were injected i.p. with PBS or Dex at 2 mg/Kg 2h before cell isolation; injected i.v. with INX231P and INX901 at 10 mg/Kg 24h before cell isolation. Spleen monocytes were placed in culture and subjected to LPS stimulation at 10 and 100 ng/ml. 24h supernatants were analyzed by ELISA (n=4 mice/group; separate ordinary one-way ANOVA as compared to PBS treated group for each LPS dose).

### Conclusions

Experiment 1 showed that In vivo Dex treatment at 2 mg/Kg efficiently prevents ex vivo monocyte activation by LPS as shown by dramatic decreases in cytokine production. EXPERIMENT 2 showed that INX231P treatment in vivo can decrease ex vivo activation of monocytes as shown by decreases in the production of some cytokines at 24h, but these effects are not observed 3 or 7 days post treatment which is consistent with the known half-life of monocytes that is in the range of 2-3 days. Additionally, ADC impact on cytokine production is due to the GC delivery to VISTA expressing cells as treatment with the unconjugated agonist counterpart antibody (same CDR) has no anti-inflammatory activity. EXPERIMENT 3, which is a repeat of EXPERIMENT 2, except looking only at 2h post Dex or 24h post ADC and unconjugated agonist treatments shows that INX231P potently decreases ex vivo activation of monocytes while the agonist antibody had no impact.

Accordingly, the experimental results show that
LPS-induced cytokine response on isolated spleen monocytes is efficiently controlled by dexamethasone.
INX231P but not INX901 treatment, efficiently controls LPS-induced cytokine response on isolated spleen monocytes when dosed 24h earlier in vivo. By day 3, no effect is observed which is consistent with the known half-life of mouse monocytes that is in the range of 2-3 days.
INX231P but not INX901 treatment, potently prevents LPS-induced ex vivo activation of spleen monocytes. In this experiment, we noted that INX231P, while delivering 10 times less GC payload than free Dex, shows high potency at high stimulation level (LPS at 100 ng/ml) whereas Dex appears to lose efficacy. Finally, as observed in Experiment 3, INX901 treatment had no effect on LPS induced cytokine responses.

Altogether the experimental results indicate that INX231P in vivo treatment can prevent monocyte ex vivo activation with a potency at least 10× superior to free steroid. By contrast, the agonist anti-VISTA antibody INX901 showed no potency in this model. Accordingly, the observed results in this experiment are entirely elicited by the steroid payload and not by VISTA modulation.

### Example 16: Impact of Anti-VISTA Drug Conjugates Effect on Transcription in Monocytes, T Regs and B Cells Depends on Target Expression

We describe herein different anti-human VISTA monoclonal antibodies linked to various glucocorticoid (GC) payloads and their in vitro and in vivo effects. In this example we assess VISTA target dependence by evaluating the impact on the transcription of a GC reporter gene FKBP5 for exemplary ADC according the invention, by evaluating the effects of 1) INX201J on monocytes and B cells vs isotype control (huIgG1si J) and free J payload and 2) INX231P (on Tregs) vs free payload (INX-SM-3).

As shown herein, treatment with anti-VISTA steroid ADC led to robust and dose dependent upregulation of FKBP5 on monocytes, cells with high expression levels of VISTA. A significant but more moderate impact was observed with Tregs that have lower VISTA expression than monocytes. Negligible impact was seen on B cells where VISTA is not expressed. No changes in FKBP5 expression were observed for either monocytes or B cells when treated with steroid conjugated isotype control.

Antibody drug conjugates (ADCs) allow for specific cell targeting of highly potent drugs to allow for efficacy while limiting toxicity. INX201 and INX231 are anti-human VISTA antibodies. In their steroid conjugated forms, INX201J and INX231P deliver steroids to VISTA expressing cells including myeloid cells, and T cells and we hoped would have little or no impact on VISTA negative cells such as B cells (Cancer Res. 74:1924-1932, 2014).

To monitor/confirm GC delivery and activity, we measured by quantitative Real Time PCR (qRT-PCR) the transcriptional activation of FKBP5 that is a direct and robust biomarker of glucocorticoid activity (JCEM 101:4305-4312, 2016). We conducted this assessment on isolated human monocytes, regulatory T cells (T regs) and B cells following in vitro treatment with ADCs.

### Materials and Methods

Monocytes or B cells were isolated from healthy donor blood samples and treated with free steroid, anti-VISTA conjugated steroid, or conjugated isotype control. RNA was isolated, and change in FKBP5 transcript level assessed by qPCR.

For monocytes vs B cell analyses, one blood donor collection was used for the single drug concentration experiment; blood from a separate single donor collection was used to assess drug dose response. For the regulatory T cell (Treg) analysis, blood from two separate donors was used.

### Test Agents

Free J payload, INX J-2 (Abzena). INX J-2 or briefly, free J payload, is a patent reported budesonide analog utilized in the full linker/payload INX J.
INX201 (Aragen, Lot #BP-3200-019-6) is a humanized anti-human VISTA antibody on a human IgG1/kappa backbone with V234A/G237A/P238S/H268A/V309L/A330S/P331S silencing mutations in the Fc region.
INX201J (Abzena, Lot #JZ-0556-025-1) is the INX201 antibody with a drug/antibody ratio (DAR) of 8.0, conjugated via full modification of the interchain disulfides. The linker/payload (J) is based on a previously reported linker/payload. It consists of a protease sensitive linker with a budesonide analog payload.
INX-SM-3 (O2H) is the budesonide analog payload utilized in the linker/payload INX P.
INX231 (ATUM, lot #72928.1.a) is a humanized anti-human VISTA antibody on a human IgG1/kappa backbone with L234A/L235A/E269R/K322A silencing mutations in the Fc region.
INX231P (Abzena, Lot #JZ-0556-017-1) is INX231 conjugated to the linker/payload INX P consisting of a protease sensitive linker with INX-SM-3, via full modification of the interchain disulfides with a DAR of 8.0.
Human IgG1siJ (Abzena, lot #JZ-0556-025-2) is an isotype control on a human IgG1/kappa backbone with E269R/K322A silencing mutations in the Fc region. The DAR ratio is 8.0, conjugated via full modification of the interchain disulfides with the INX J linker/payload.

### Additional Reagents.

Ficoll-Paque Plus (GE Healthcare cat #17-1440-03)
RPMI 1640 without L-glutamine (VWR cat #16750-084)
Penicillin/Streptomycin/Glutamine (ThermoFisher cat #10378016)
1M Hepes (Gibco cat #15630-080)
Human AB serum (Valley Biomedical cat #HP1022HI)

### PBMCs Preparation

Human PBMCs were isolated under sterile conditions from apheresis cones obtained from the Blood Donor Program at the Dartmouth Hitchcock Medical Center from deidentified healthy human donors.

The blood was transferred to a 50 ml Falcon tube and diluted with PBS to 30 ml. 13 ml of Histopaque 1077 (Sigma Aldrich) was slowly layered under the blood, and tubes were centrifuged at 850×g for 20 min at RT with mild acceleration and no brake.

Mononuclear cells were collected from the plasma/Ficoll interface, resuspended in 50 ml of PBS and centrifuged at 300×g for 5 min. Cells were resuspended in PBS and counted.

### Assay Protocol

The various immune populations were isolated using different cell isolation kits and following manufacturer instructions:
EasySep Human monocyte enrichment kit without CD16 depletion (StemCell cat #19058)
Pan B cell isolation kit, human (Miltenyi Biotec, 130-101-638).
EasySep™ Human CD4+CD127 low CD49d-regulatory T cell enrichment Kit (StemCell cat #19232)

Monocytes, B cells or Tregs were plated (from single donors) at 2×10\hat{ }6 cells per well in a 12-well plate in RPMI, 10% human AB serum, 10 mM Hepes, 1× Penicillin/Streptomycin/Glutamine.

For single dose experiments, cells were treated with 20 nM free J payload or INX-SM-3 payload or the molar payload equivalent of huIgG1si J, INX201J or INX231P (the linked form of INX-SM-3).

For dose response, serial dilutions resulting in 100, 20, 5, 0.5, 0 nM of free J payload or the molar payload equivalent of INX201J. For the 0 nM point, unconjugated INX201 equivalent to the amount of antibody used for the 100 nM molar payload equivalent of INX201J was used (e.g., 12.5 nM unconjugated antibody). A no treatment well was used as a control.

Plates were incubated for 1 day at 37° C.

Cells were then harvested and wells for each condition were pooled post harvesting to allow sufficient RNA for subsequent qRT-PCR analysis.

### RNA Preparation and Real Time PCR

After one wash with PBS, RNA was isolated from cell pellets using either the RNeasy Plus Mini kit (Qiagen, PN: 74136) or NucleoSpin RNA Plus (Macherey-Nagel #740984.250). RNA was isolated following manufacturer's instructions and eluted in in 30 or 40 μl H2O (RNase/DNase free). RNA concentration was assessed by UV spectroscopy using a Nanodrop 2000.

Reverse transcription was done using Taqman reverse transcription reagents (#N8080234) and following manufacturer's instructions.

Quantitative Real-Time PCR was done using Taqman master mix 2× kit (#4369016) and run on a QuantStudio3 from Applied Biosystem. Primers used:

### Experiment 1 and Experiment 2

i. Life Technologies Cat #433111182 Hs01561006_m1 (FKBP5)
ii. Life Technologies cat #HS99999905_m1 (GapDH)

### Experiment 3 AND EXPERIMENT 4

i. TaqMan Gene Expression Assay (FAM-MGB); Assay ID: Hs01561006_m1 (FKBP5)
ii. TaqMan Gene Expression Assay (FAM-MGB); Assay ID: Hs01922876_u1 (GapDH)

Ct data were converted to ΔCt and ΔΔCt or Log 2 fold changes compared to untreated control.

### Results

### Experiment 1

In this experiment, we evaluated the necessity of target expression for steroid delivery by an ADC as assessed by induction of FKBP5 transcription in monocytes as a VISTA positive cell population and B cells as a VISTA negative population. Free steroid was added as a positive control for steroid impact on FKBP5 levels for a particular cell type. Free steroid (free J payload), J linker-payload conjugated anti-VISTA (INX201J) or isotype control (huIgG1si J) were dosed to provide the same molar equivalent of payload (20 nM).

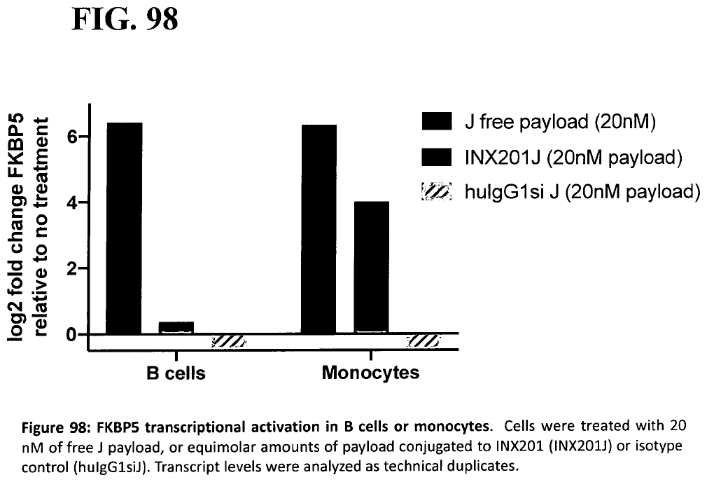

As shown in FIG. 98, a robust increase in FKBP5 transcription was observed with free J payload in both monocytes and B cells relative to a no-treatment control. However, robust FKBP5 transcription was observed in monocytes but not B cells when treated with anti-VISTA conjugated payload (INX201J). No FKBP5 transcription was detected in both cell types when treated with payload conjugated isotype control (HuIgG1si). Particularly FIG. 98 shows FKBP5 transcriptional activation in B cells or monocytes in cells which were treated with 20 nM of free J payload, or equimolar amounts of payload conjugated to INX201 (INX201J) or isotype control (huIgG1siJ). Transcript levels were analyzed as technical duplicates.

### Experiment 2

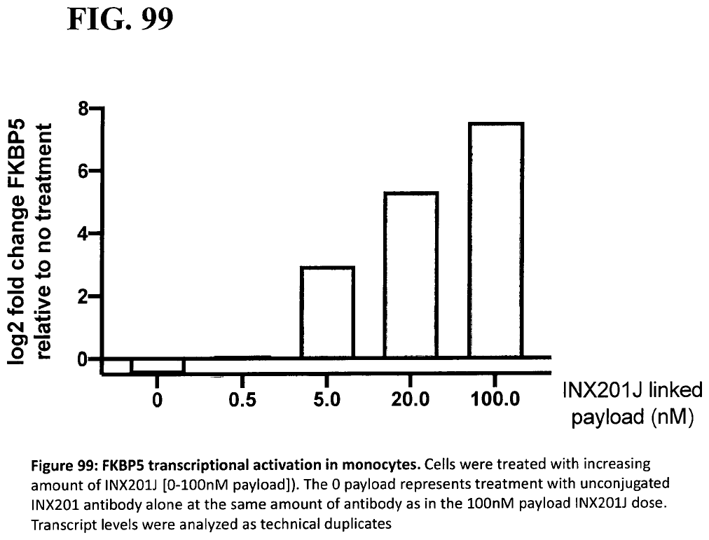

In the experiment in FIG. 99, we expanded upon EXPERIMENT 1 by assessing the dose dependent effect of treatment with steroid linked anti-VISTA (INX201J) on monocytes (high VISTA expression). Cells were treated with a serial dilution of INX201J (100 nM to 0 nM payload). For the 0 nM concentration only, INX201 unconjugated antibody was treated with the equivalent amount of antibody as present for the 100 nM payload samples. Specifically, as 12.5 nM ADC delivers 100 nM payload, for the 0 nM sample, unconjugated INX201 was added to 12.5 nM. As shown in FIG. 99, treatment of monocytes with INX201J leads to robust dose dependent effects. In FIG. 99 FKBP5 transcriptional activation in monocytes is shown in cells which were treated with increasing amount of INX201J [0-100 nM payload]). The 0 payload represents treatment with unconjugated INX201 antibody alone at the same amount of antibody as in the 100 nM payload INX201J dose. Transcript levels were analyzed as technical duplicates.

### Experiment 3

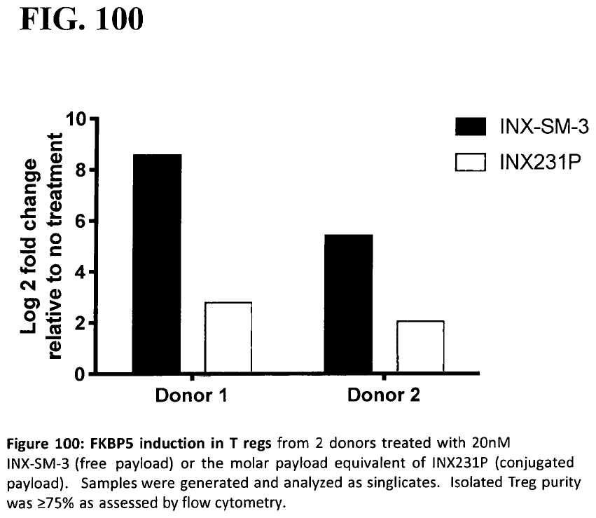

In the experiment in FIG. 100, we assessed the impact of a second anti-VISTA steroid conjugate (INX231P) on FKBP5 transcription induction in VISTA expressing Tregs. As shown in FIG. 100, treatment of Tregs with 20 nM of free payload (INX-SM-3) or the molar payload equivalent of anti-VISTA conjugated payload (INX231P), lead to increased FKBP5 transcription. This experiment was conducted with 2 different donors and the isolated Treg purity was ≥75%.

### Experiment 4

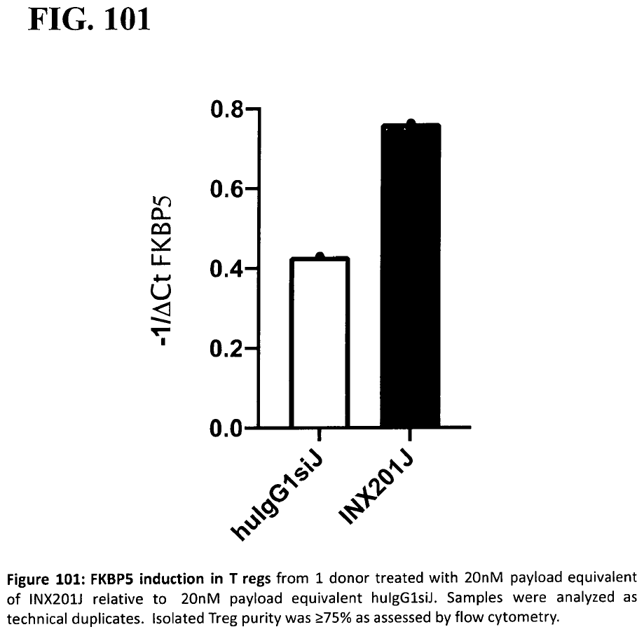

In the experiment in FIG. 101, we assessed the impact of an anti-VISTA steroid conjugate (INX201J) vs isotype control conjugated with the same linker/payload (huIgG1si J) on FKBP5 transcription induction in Tregs. Increasing-1/delta Ct, as shown with INX201J treatment, represents increased transcript abundance relative to housekeeping (GapDH). Treatment of Tregs with INX201J delivering 20 nM steroid payload lead to a 2.1 fold increase in FKBP5 transcription vs conjugated isotype control (Fold change=2\hat{ }(ΔCt INX201J-ΔCt huIgG1si J)). This experiment was conducted with 1 donor and the isolated Treg purity was ≥75%. Specifically, the data in FIG. 101 shows FKBP5 induction in T regs from 1 donor treated with 20 nM payload equivalent of INX201J relative to 20 nM payload equivalent huIgG1si J. Samples were analyzed as technical duplicates. Isolated Treg purity was ≥75% as assessed by flow cytometry.

### Conclusions

The data demonstrate that anti-VISTA antibodies conjugated to steroid specifically induce FKBP5 transcription in monocytes and Tregs, but not in B cells indicating that payload delivery is specific and target dependent.

While all cell types analyzed showed robust responses to free payload, only VISTA expressing cell types (monocytes/Tregs) show moderate to strong responses when treated with 20 nM of anti-VISTA steroid conjugates. Additionally, isotype control ADC showed little to no induction of FKBP5 when compared to no treatment controls.

Target requirement for GC effect is supported by a robust dose dependent impact on VISTA expressing cells and limited to no impact on non-VISTA expressing cells by anti-VISTA ADCs.

### Example 17: RNA Expression Various Immune Cells by Antigens Targeted by Exemplary Anti-Inflammatory Drug Conjugates

As afore-mentioned the subject anti-inflammatory drug conjugates are believed to possess a superior attributes in relation to previous anti-inflammatory drug conjugates in part because of the expression or absence of expression of VISTA on specific immune and non-immune cells compared to antigens which have been targeted by previous anti-inflammatory drug conjugates.

This is suggested by their reported RNA expression profiles. In particular the inventors initially compared RNA expression of VISTA and other immune cell targets on immune and non-immune cells based on a comprehensive review of “Human Protein Atlas Version 20.1 and Berglund L et al., “A genecentric Human Protein Atlas for expression profiles based on antibodies”, Mol Cell Proteomics, Vol. 7 (10): 2019-2027 (Oct. 1, 2008) (https://www.proteinatlas.org).

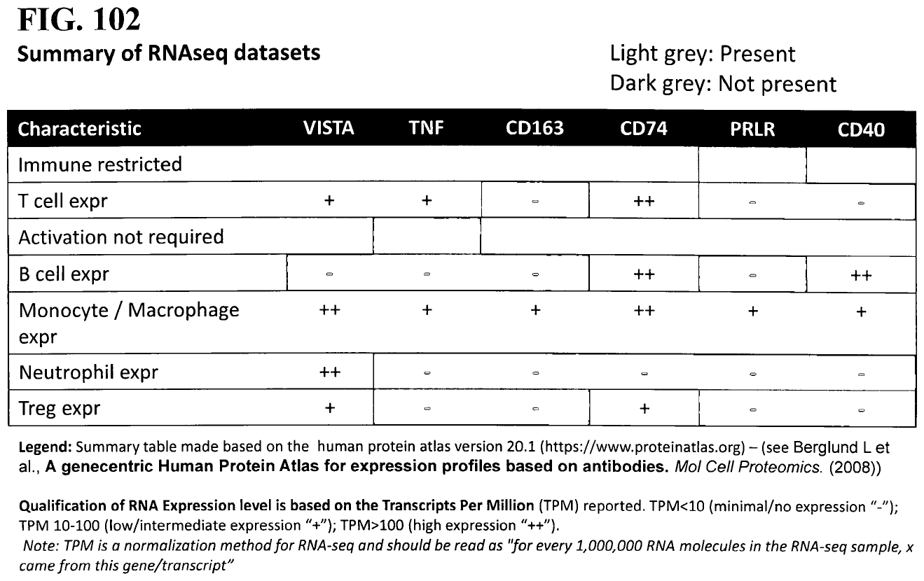

Based on this analysis the inventors prepared a Consensus Dataset from the reported human tissue/cell RNAseq data from Human Protein Atlas Version 20.1 and Berglund et al. (Id). The results of this comparison are shown in FIG. 102. Particularly, FIG. 102 summarizes the consensus RNA expression levels by different cells for VISTA and other ADC targets (CD40, TNF, PRLR, CD174) based on the “Transcripts Per Million” (TPM) reported wherein a TPM<10 represents (minimal/no expression “−”); a TPM 10-100 represents (low/intermediate expression “+”); and a TPM>100 (high expression “++”). As is known in the art TPM is a well-known normalization method for RNA-seq and should be read as “for every 1,000,000 RNA molecules in the RNA-seq sample, x came from this gene/transcript”.

As shown in the FIG. 102, VISTA is the only target for which RNAs are expressed broadly on activated and non-activated myeloid cells (monocytes, macrophages, neutrophils), T cells, dendritic cells, NK cells, and eosinophils (data not shown, see e.g., “The immune checkpoint molecule VISTA regulates allergen-specific Th2-mediated immune responses”, Tatsukuni Ohno et al., International Immunology, Volume 30, Issue 1, January 2018, Pages 3-11); by contrast TNF expression is relatively low for most cell types and moreover is only expressed on activated immune cells; CD163 is expressed by myeloid cells but not by lymphocytes; CD40 misses T cells; PRLR not widely expressed on immune cells and not immune-restricted; and CD74 misses neutrophils. (This is significant since neutrophils are important during the beginning (acute) phase of inflammation, particularly during bacterial infection, environmental exposure, and some cancers and indeed are one of the first responders of inflammatory cells to migrate toward the site of inflammation via chemotaxis. (Yoo S K et al., (November 2011). “Lyn is a redox sensor that mediates leukocyte wound attraction in vivo”, Nature, 480 (7375): 109-12).

With respect to the foregoing, while these reported RNA expression levels by different immune cells are of interest they do not provide actual evidence as to the comparative putative efficacy of these antigens as ADC targets. Rather, this can only be reasonably assessed by actual surface protein expression levels of these targets on different immune cells and experimental evidence that VISTA ADCs effectively target and are efficacious in different immune cells (i.e., provide for the internalization and release of therapeutically effective amounts of active inflammatory drugs such as steroids into one or more of these different types of immune cells).

### Example 18: Comparison of Surface Expression of VISTA by Various Immune Cells Compared to Antigens Targeted by Exemplary Anti-Inflammatory Drug Conjugates and Antibody Binding Capacity of Anti-VISTA, Anti-CD74, Anti-CD163, and Anti-mTNFα Antibodies to Human Peripheral Blood Mononuclear Cells and Whole Blood

The surface antigen density of VISTA, CD74, CD163 and membrane TNFα (mTNFα) was assessed by flow cytometry on naive human peripheral blood mononuclear cells (PBMCS) and in whole blood. As indicated below the data show that when compared to CD74, CD163 and mTNFα:
Only VISTA is expressed in steady state on human CD8+ and CD4+ T cells
VISTA shows the highest antigen density on CD14+ monocytes
Surface mTNFα was not detected on any cell type tested

VISTA is highly expressed on most hematopoietic cells, particularly on myeloid and T cells. The objective of the present studies was to evaluate the antigen density of VISTA, CD74, CD163, and mTNFα on both human PBMCs and leukocytes from whole blood.

### Materials and Methods

### Experimental Design

The binding of directly labelled antibodies to human cells (PBMCs) or whole blood leukocytes from multiple donors were determined by flow cytometry and the antigen density calculated using calibration beads.

### Reagents

### Antibodies:

Anti-VISTA GG8 (Aragen lot #AB131122-3) is a chimeric anti-human VISTA antibody on a wildtype human IgG1/kappa backbone and was generated at ImmuNext. The GG8 clone was conjugated with Alexa Fluor 647 dye following manufacturer's instructions for labelling and purification (Invitrogen, cat #A20186). All remaining antibodies were purchased from BioLegend, unless stated otherwise, and used as is including:
CD127 Brilliant Violet 421 clone A019D5,
CD14 PE-Cy7 clone M5E2,
CD20 Brilliant Violet 510 clone 2H7,
CD4 APC-Cy7 clone OKT4,
CD163 Alexa Fluor 647 clone GHI/61,
CD25 FITC clone BC96,
CD74 Alexa Fluor 647 clone 332516 (R&D Systems),
CD8 PE clone BW135/80 (Miltenyi), mTNFα Alexa Fluor 647 clone mAb11.

### Other Reagents:

The calibration beads (Quantum Simply Cellular Mouse IgG) were purchased from Bangs Laboratories and used following manufacturer's protocol.

### PBMC Preparation

Human PBMCs were isolated under sterile conditions from apheresis cones obtained from the Blood Donor Program at the Dartmouth Hitchcock Medical Center from healthy unrelated human donors. First, the blood was transferred to a 50 ml Falcon tube and diluted with PBS to 30 ml. 13 ml of Histopaque 1077 (Sigma Aldrich) was slowly layered under the blood, and tubes were centrifuged at 850×g for 20 min at RT with mild acceleration and no brake.

Mononuclear cells were collected from the plasma/Ficoll interface, resuspended in 50 ml of PBS and centrifuged at 300×g for 5 min. Cells were resuspended in PBS and counted.

### Whole Blood Preparation

Fresh blood was drawn at Dartmouth Hitchcock Medical Center from healthy unrelated human donors and staining done on whole blood.

### Antibody Binding and Analysis

### PBMC Staining

PBMCs were resuspended in PBS/0.2% BSA buffer containing human Fc blocking reagent (eBioscience, 14-9161-73) and 106 cells/well were then distributed to a 96-well plate. An antibody cocktail was prepared and PBMCS were stained for 30 min on ice to limit internalization, washed twice with PBS.

### Whole Blood Staining

100 μl of blood was stained in a deep well 96-well plate and antibody cocktail was added directly. After 30 min incubation the erythrocytes were lysed with 1 ml of ACK buffer (Gibco) for 10 min. Blood was centrifuged and blood leukocytes were transferred to a 96-well plate, washed with PBS and analyzed.

### Binding Quantification

Quantification beads were stained with anti-VISTA, anti-CD74, anti-CD163, and anti-mTNFα following manufacturer's protocol. Cells and beads were analyzed by fluorescence associated cell sorting (FACS), using a Macsquant (Miltenyi) flow cytometer and FlowJo for analysis. Antibody binding capacity was calculated using QuickCal analysis template provided with the Quantum beads.

All graphs were prepared with GraphPad (Prism).

### Results

### Evaluation of Test Antibody Binding on PBMCS

To evaluate the antigen density on cell populations, human PBMCs from 5 different donors were incubated with the mAbs and analyzed by flow cytometry. The median fluorescence was normalized by substracting background signal and calibrated against the quantification beads with known antibody binding capacity. Cell populations were identified as CD20⁺ B cells, CD14⁺ SSC^{high} monocytes, CD8⁺ and CD4⁺ T cells, and CD4⁺ CD25⁺ CD127^{low} T regulatory cells (T regs). All values are reported as mean±SD.

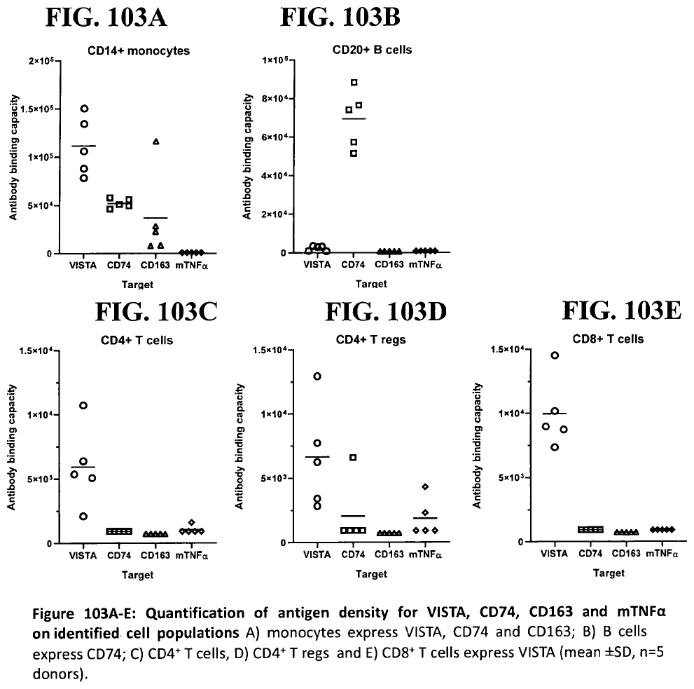

As shown in FIG. 103A, CD14+ monocytes expressed 3 targets at high levels, with VISTA being the most abundant with an antibody binding capacity or ABC=111587±30502, followed by CD74 (ABC=52001±4765), and CD163 (ABC=36671±12339) (FIG. 103A). Of note, for CD163 the mean was elevated due to one outlier donor showing 5x higher expression than the remaining 4.

As shown in FIG. 103B, only CD74 was detected on B cells, and 69574±14997 molecules were quantified. It can be seen that VISTA was the only protein expressed on non-activated T cells, with mean density of 5938±3113 molecules on CD4⁺ (FIG. 103C), 6641±4059 on T regs (FIG. 103D), and 9958±2741 molecules on CD8⁺ (FIG. 103E).

In naïve PBMCs, mTNFα was not detected above background level. The absence of mTNFα was confirmed by negative staining with a second mTNFα antibody (R&D Systems, Adalimumab biosimilar, clone Hu7). mTNFα was also not detected on cells activated with LPS (data not shown). Specificity of commercially obtained anti-TNFα antibody was determined by manufacturer and confirmed internally via ELISA (data not shown).

FIG. 103A-E summarizes the quantification of antigen density for VISTA, CD74, CD163 and mTNFα on identified cell populations A) monocytes express VISTA, CD74 and CD163; B) B cells express CD74; C) CD4⁺ T cells, D) CD4⁺ T regs and E) CD8⁺ T cells express VISTA (mean±SD, n=5 donors).

### Analysis of the Antibody Binding on Whole Blood Leukocytes

Neutrophils are an essential part of the immune system that is missing from the PBMCS preparation. Therefore, whole blood leukocytes from 3 healthy donors were also examined and the antigen expression on cell populations evaluated. Similarly to PBMCS, whole blood was stained with monoclonal antibody cocktail and analyzed by FACS. The median fluorescence was normalized by substracting background signal and calibrated against the quantification beads with known antibody binding capacity.

Cell populations were identified as CD20⁺ B cells, CD14⁺ SSC^{high} monocytes, CD66b⁺ SSC^{high} neutrophils, CD8⁺ and CD4⁺ T cells, CD4⁺ CD25⁺ CD127^{low} T regulatory cells (T regs). All values are reported as mean±SD.

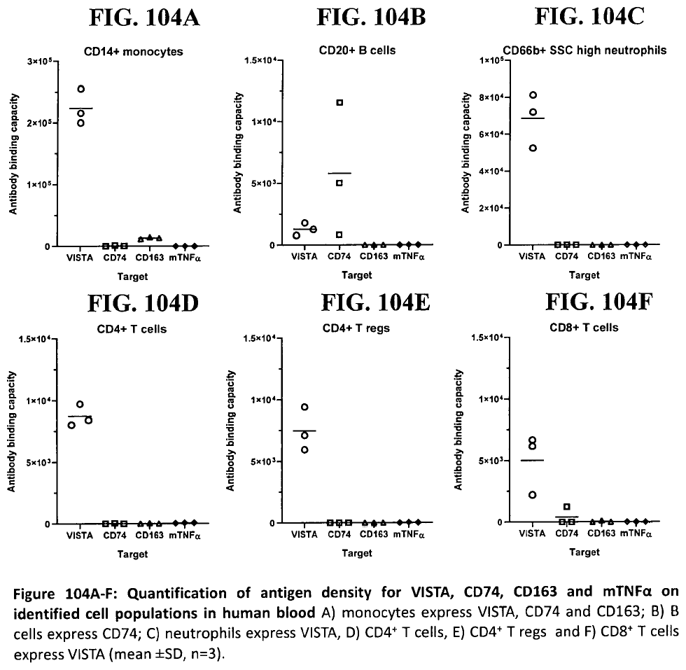

As was observed on PBMCs, VISTA was the most abundant on CD14+ monocytes (ABC=223674±16503), CD163 expression was maintained at 13126±790 molecules, but the expression of CD74 was much lower than in PBMCs (ABC=562±338) (FIG. 104A). The expression of CD74 on CD20⁺ B cells was very variable between donors (5800±3121). A minimal signal was similarly observed for VISTA (ABC=1280±291) (FIG. 104B). Neutrophils showed high levels of VISTA expression (ABC=68571±14731) (FIG. 104C) while the other targets of interest were not detected. Finally, VISTA expression on T cells was confirmed in the whole blood as well, with 8717±886 molecules detected. VISTA expression on T cells was confirmed in the whole blood as well, with 8717±886 molecules detected on CD4⁺ (FIG. 104D), 7486±1767 on T regs (FIG. 104E), and 5012±2438 on CD8⁺ (FIG. 104F). FIG. 104A-F summarizes the quantification of antigen density for VISTA, CD74, CD163 and mTNFα on identified cell populations in human blood A) monocytes express VISTA, CD74 and CD163; B) B cells express CD74; C) neutrophils express VISTA, D) CD4+ T cells, E) CD4+ T regs and F) CD8+ T cells express VISTA (mean±SD, n=3).

### VISTA Expression on Activated Immune (Monocytes) Compared to Other Targets

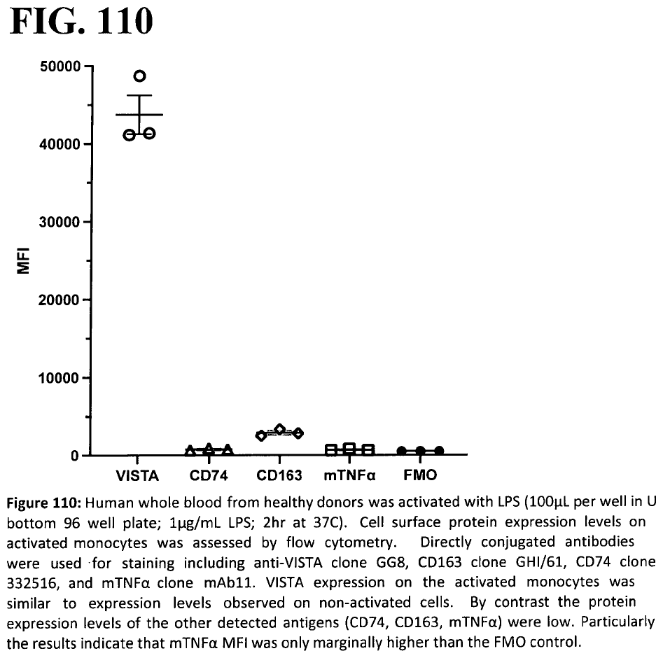

Additionally an experiment was conducted which compared the expression of VISTA to other antigens on activated immune cells (monocytes). As shown in FIG. 110 the expression of VISTA on activated immune cells (particularly monocytes) was compared to the expression levels of other proteins (specifically proteins which have been targeted with other steroid ADCs) on activated immune (monocyte) cells.

In the experiment human whole blood from healthy donors was activated with LPS (100 μL per well in U bottom 96 well plate; 1 μg/mL LPS; 2 hr at 37° C.). Cell surface protein expression levels on activated immune cells was assessed by flow cytometry. Directly conjugated antibodies were used for staining again included anti-VISTA clone GG8, CD163 clone GHI/61, CD74 clone 332516, and mTNFα clone mAb11.[Anti-VISTA clone GG8 (Aragen lot #AB131122-3) is a chimeric anti-human VISTA antibody on a wildtype human IgG1/kappa backbone and was generated at ImmuNext; the GG8 clone was conjugated with Alexa Fluor 647 dye following manufacturer's instructions for labelling and purification (Invitrogen, cat #A20186). All remaining antibodies were purchased and used as is: CD163 Alexa Fluor 647 clone GHI/61 (Biolegend), CD74 Alexa Fluor 647 clone 332516 (R&D Systems), mTNFα Alexa Fluor 647 clone mAb11 (Biolegend)].

As shown, in FIG. 103, VISTA expression patterns on activated monocytes were similar to expression levels observed on non-activated monocytes whereas the other detected proteins the expression levels which were detected on activated monocytes were low. Moreover, mTNFα MFI was only marginally higher than the Fluorescence Minus One (FMO) control. These results further indicate that the subject VISTA ADCs can be used to target both activated and non-activated immune cells, e.g., monocytes and other myeloid cells which express VISTA at high levels (as well as other immune cells which constitutively express VISTA such as those previously identified, e.g., eosinophils, dendritic cells, macrophages, myeloid cells, CD4 T cells, CD8 T cells, Tregs, NK cells, monocytes, neutrophils, and the like).

### Conclusions

The data summarized in FIGS. 103, 104, 110 and Table 6 below show that:
Human VISTA is the most robust ADC target protein with high expression levels on monocytes, neutrophils and T cells. Of note, though some RNA databases describe high levels of CD74 transcript in T cells, (Berglund L et al., “A genecentric Human Protein Atlas for expression profiles based on Mol antibodies”, Cell Proteomics. (2008) DOI: 10.1074/mcp.R800013-MCP200) we did not observe surface expression of CD74 on T cells.
VISTA is expressed at much higher levels on activated immune cells (monocytes) than other targets analyzed and the expression of VISTA on activated and non-activated immune cells (e.g., monocytes) is similar further indicating that the subject VISTA ADCs can be used to target both activated and non-activated immune cells, e.g., monocytes (as well as other immune cells previously identified such as eosinophils, NK cells, cells, macrophages, CD4 T cells, CD8 T cells, Tregs, NK cells, neutrophils, and the like).
CD74 is consistently detected on B cells in both PBMCs and whole blood but only on monocytes from PBMCs.
CD163 is expressed only on monocytes from PBMCs.
mTNFα was not detected on any cell population analyzed.
VISTA is the only protein expressed on non-activated (naïve) T cells, with mean density of 5938±3113 molecules on CD4⁺ cells, 6641±4059 on T regs, and 9958±2741 molecules on CD8⁺ cells.

TABLE 6
Summary of the surface expression on human cell populations.

| | Target | | | | | | | |
|---|---|---|---|---|---|---|---|---|
| Cell | VISTA | | CD74 | | CD163 | | mTNFα | |
| population | PBMCS | WB | PBMCS | WB | PBMCS | WB | PBMCS | WB |
| Monocyte | +++ | +++ | ++ | | ++ | ++ | | |
| Neutrophil | Na | ++ | Na | | Na | | na | |
| B cell | | | ++ | + | | | | |
| CD4+ T cell | + | + | | | | | | |
| CD4+ Treg | + | + | | | | | | |
| CD8+ T cell | + | + | | | | | | |

TABLE 6 contains a summary of the surface expression of different antigens including VISTA on human immune cell populations. Expression of the analyzed surface targets was categorized as present (light grey) or absent from the cell surface (dark grey); based on the normalization to the quantification beads, + corresponds to 1000-10000 molecules, ++ corresponds to 10000-100000, +++ corresponds to above 100000; WB—whole blood, PBMCS—peripheral blood mononuclear cells; na—not applicable.

As afore-mentioned the subject anti-inflammatory drug conjugates are believed to possess superior attributes in relation to previous anti-inflammatory drug conjugates in part because of the expression or absence of expression of VISTA on specific immune and non-immune cells compared to antigens which have been targeted by previous anti-inflammatory drug conjugates.

Based on these results, since VISTA is only expressed by immune cells, unlike some other targets such as PRLR which are not immune restricted, VISTA ADCs should be less prone to eliciting toxicity to non-target cells. Moreover, because VISTA is constitutively expressed by naive immune cells and T cells in particular, unlike some other ADC targets such as TNF, VISTA ADCs may be preferred for use in the treatment of chronic autoimmune and inflammatory diseases since VISTA ADCs should maintain a constant level of efficacy (i.e., will be effective during activation and non-activation) thereby potentially reducing the likelihood of recurrence of inflammation, and/or may reduce the level of inflammation during reoccurrence of inflammation or autoimmunity. This is therapeutically significant as many autoimmune/inflammatory diseases are remitting/relapsing and consequently a significant clinical objective of drugs and biologics used to treat such conditions is to provide a therapeutic regimen whereby the disease is effectively managed both during remission and relapse such that the patient does not suffer tissue damage.

Moreover, of these ADC targets only VISTA is expressed on neutrophils. This is significant since neutrophils are important during the beginning (acute) phase of inflammation, particularly during bacterial infection, environmental exposure, and some cancers and indeed are one of the first responders of inflammatory cells to migrate toward the site of inflammation via chemotaxis. (Yoo S K et al., (November 2011). “Lyn is a redox sensor that mediates leukocyte wound attraction in vivo”. Nature. 480 (7375): 109-12). Also, since these cells are expressed in the early phase of inflammatory responses VISTA ADCs are expected to have a rapid onset of action (which in fact is shown herein).

Of yet additional therapeutic significance, because VISTA is also not expressed on B cells (unlike some other ADC targets such as CD40 and CD74), VISTA ADCs should not affect B lymphocytes during treatment. Accordingly VISTA ADCs may preserve humoral immunity during treatment, which may reduce the likelihood of the subject developing an infection or even cancer during treatment. (Because steroids are potent immunosuppressives a risk associated therewith, particularly during chronic usage, is the risk that the treated subject may develop a lethal infection or malignancy during treatment).

Also, of these ADC targets only VISTA appears to be constitutively expressed by naïve Tregs, CD4⁺ T and CD8⁺ T cells. This is significant particularly since these cells are involved in inflammatory response and further since Tregs have recently been reported to be highly significant to the efficacy of steroids. (See Buttgereit, Frank and Timo Gaber, Timo; Cellular and Molecular Immunology, “New insights into the fascinating world of glucocorticoids: the dexamethasone-miR-342-Rictor axis in regulatory T cells”, Vol. 18, 520-522 (2021); and Immunity, “Anti-inflammatory Roles of Glucocorticoids Are Mediated by Foxp3+ Regulatory T Cells via a miR-342-Dependent Mechanism, Vol. 53 (2): 581-596 (September 2020); Braitch M. et al., Acta Neurol Scand., “Glucocorticoids increase CD4+CD25high cell percentage and Foxp3 expression in patients with multiple sclerosis”, 2009 April; 119 (4): 239-245).

In fact experimental evidence contained herein demonstrates that VISTA ADCs effectively target and are efficacious in these different types of immune cells (i.e., provide for the internalization of therapeutic (anti-inflammatory) amounts of steroids into different types of immune cells).

### Example 19: PK vs PD Summary

As afore-mentioned the subject anti-inflammatory drug conjugates provide for PD durations which are much more prolonged than expected given the short PK of the anti-VISTA antibody comprised in the conjugate. The PK, PD and Kd values for exemplary anti-VISTA antibodies and ADCs containing according to the invention are summarized in TABLE 7.

The CDR and variable sequences for the antibodies identified in TABLE 7 are found in FIGS. 8, 10 and 12. The PD or potency which refers to FKBP5 in the TABLE is defined as 2 fold induction of FKBP5 over PBS in macrophages at 14 days post dosing. The PD or potency which refers to “Cytokine reduction” in the TABLE is defined as 20% reduction in TNFα at 7 days post dosing in ex vivo macrophage activation assay. These assays are exemplified in Example 6.

TABLE 7

| | | | | | | PK-VISTA knock-in mouse (t_{1/2}, hours) | | | | | |
|---|---|---|---|---|---|---|---|---|---|---|---|
| | ADC (IgG1 | | | | | Silent | | | | PD (macrophage | |
| | with INX | | | | | IgG1 | | | | assay) | |
| ADC (IgG1 | and LALA | | | | | (INX | Silent IgG1 | | | FKBP5 | Cytokine |
| INX Silent | silent | Agonist | Antagonist | Binding | Kd | LALA or | (INX mutations) | | | readout | readout |
| mutations) | mutations) | (IgG2) | (IgG1) | Bin | (nM) | LALA) | conjugated | Agonist | Antagonist | (day) | (day) |
| INX200 | INX201 | INX908 | VSTB92 | 1 | 0.09 | 2.3 h | 2.3 h | 3.6 h | Not tested | 14 (INX | 7 (INX |
| | | | | | | (INX200) | (INX200A) | | | 201 w J | 201 w J |
| | | | | | | | | | | and P | and P |
| | | | | | | | | | | linker | linker |
| | | | | | | | | | | payload) | payload) |
| | INX231 | INX901 | VSTB56 | 2 | 0.02 | 4.7 h | Not tested | 11.9 h | Not tested | 14 | 7 (INX231 |
| | | | | | | | | | | (INX231 | w J, P, R, |
| | | | | | | | | | | w J and | S, V, W |
| | | | | | | | | | | P linker | linker |
| | | | | | | | | | | payload) | payloads) |
| | INX233 | INX903 | VSTB95 | 1 | 0.13 | Not | Not tested | 3.7 h | Not tested | | 7 |
| | | | | | | tested | | | | | (INX233P) |
| | INX234 | INX904 | VSTB103 | 1 | 0.64 | 5.5 h | Not tested | 31.5 h | Not tested | 14 | 7 (INX234 |
| | | | | | | | | | | (INX234 | w J and P |
| | | | | | | | | | | w J and | linker |
| | | | | | | | | | | P linker | payload) |
| | | | | | | | | | | payload) | |
| | INX237 | INX907 | VSTB66 | 2 | 0.08 | 56.5 h | Not tested | 24.2 h | Not tested | 14 | Not |
| | | | | | | | | | | (INX237 | tested |
| | | | | | | | | | | w J | |
| | | | | | | | | | | linker | |
| | | | | | | | | | | payload) | |

The data in TABLE 7 shows that exemplary antibodies (which all bind to human VISTA expressing immune cells at physiologic pH and which possess short pKs, notwithstanding provide for long PDs, i.e., as would be anticipated for an antibody with a longer (and more typical) PK for a therapeutic antibody. This data substantiates that the subject ADCs should be suitable for uses wherein prolonged efficacy is desired.

### Example 20: Anti-VISTA Antibody Drug Conjugates have Target Dependent Effects in Blood but not in Tissues with Little or No Target Expressing Cells

These studies were conducted to evaluate the targeting specificity of the antibody drug conjugates (ADC) INX231P and INX234P to VISTA expressing tissues. To monitor/confirm GC delivery and activity, genome wide transcriptional impact via RNA sequencing (RNAseq) was measured according to Vermeer et al. (2003) Glucocorticoid-induced increase in lymphocytic FKBP51 messenger ribonucleic acid expression: a potential marker for glucocorticoid sensitivity, potency, and bioavailability. J Clin Endocrinol Metab. January; 88 (1): 277-84. As shown in earlier examples, INX201J results in rapid and long-term induction of FKBP5, in VISTA-expressing tissues such as spleen. In contrast, non-VISTA-expressing tissues displayed little to no FKBP5 induction. The analyzes were conducted in cynomolgus monkey, comparing VISTA expressing (blood) and non-expressing tissues (brain, white adipose and bones) (EXPERIMENT 1) and in mouse bones (EXPERIMENT 2).

As disclosed in previous examples herein we have demonstrated that ADCs according to the invention are potent for prolonged duration. Particularly we show that treatment with exemplary ADCs, i.e., INX231P and INX234P treatments lead to rapid and long-term (>4 days) induction of FKBP5, a direct transcriptional target of GC in VISTA-expressing tissues such as spleen. In contrast, non-VISTA-expressing tissues displayed little or no FKBP5 induction.

In this example, we show the target specific impact of treatment by the inventive ADCs using RNAseq to assess global transcriptional changes. The analyses were conducted in non-human primates (NHP, cynomolgus monkey), and compared transcription in VISTA expressing (blood) and non-expressing tissues (brain, white adipose and bones) and in mouse bones. Also we assessed intracellular accumulation of released payload (INX-SM-3), cysteine modified linker payload (INXP-cys) and free dexamethasone across multiple VISTA expressing and non-expressing tissues via mass spectrometry. Specifically, experiments were conducted in C57BI/6 or in human VISTA knock-in (hVISTA KI) mice which have the human VISTA cDNA knocked-in in place of the mouse VISTA gene, and express human VISTA both at RNA and protein levels with the same expression pattern as mouse VISTA. Briefly, for mice, INX231P, PBS, or free dexamethasone (Dex) were delivered via intraperitoneal (i.p.) injection. After 20h for INX231P and 2h for Dex treatments, bones were isolated, bone marrow flushed and RNA extracted.

For the NHP (cynomolgus monkey) studies, INX234P, vehicle or free Dex were delivered intravenously (i.v.). Half of the animals for each group was sacrificed at 24h (except for the vehicle group) and the other half after 7 days. At both time points, animals were bled and then subjected to necropsy. Tissues were collected and RNA or protein/peptides extracted.

For both studies, genome wide transcriptional levels were evaluated by RNAseq (Admera Health) and intracellular payload levels by mass spectrometry (Quintara Biosciences).

The objective of these studies was to evaluate the impact of our anti-VISTA steroid ADCs on gene expression in target bearing and non-target bearing tissues in both mice and NHP. In addition, we assessed the accumulation of released payload in target and non-target bearing tissues in NHP via mass spectrometry (Quintara Biosciences).

Genome wide changes in transcript expression were assessed via RNAseq (Admera Health). Transcripts specifically identified in the literature to be impacted by glucocorticoid action were monitored. One of these transcripts is FKBP5, a sensitive and early GC response gene (Vermeer et al. (2003) Glucocorticoid-induced increase in lymphocytic FKBP51 messenger ribonucleic acid expression: a potential marker for glucocorticoid sensitivity, potency, and bioavailability. J Clin Endocrinol Metab. January; 88 (1): 277-84)). We have shown (ADCINVIVO.04) that Dex treatment causes dramatic increases in FKBP5 messenger RNA in VISTA expressing cells 2-4h post treatment, but that the transcriptional impact is gone by 24h. In contrast, the ADC's impact on FKBP5 transcription is long-lasting, with peak induction at 20h post treatment but signal is still detectable for 3 days in monocytes and 14 days in macrophages.

In these experiments, the anti-VISTA antibodies INX231 and INX234 with the INX P linker payload as described herein or free Dex delivered in vivo respectively were used. Various tissues were collected, and RNA isolated and transcriptional impact of treatment assessed for both mouse and cynomolgus tissues. Intracellular released payload and linker payload accumulation was assessed via mass spectrometry (Quintara Biosciences) for both Dex- and INX234P-treated groups across a variety of cynomolgus tissues. The Materials and Methods used are described in specific detail below.

### Materials and Methods

### Methods

### Experiment 1

Dex was injected i.p. at 2h before mouse euthanasia and bone isolation, which corresponds to peak FKBP5 induction. The experiment was conducted in C57BI/6 female mice.

The ADC (INX231P) was injected i.p. 20h before mouse euthanasia, to provide sufficient time for ADC processing and peak potential gene induction. The experiment was conducted in hVISTA KI female mice.

A control group injected with PBS only was included to define transcript baseline. Following euthanasia, bones were isolated, the bone marrow flushed, and bones snap frozen in liquid nitrogen prior to RNA isolation and RNAseq analysis (Admera Health).

Vehicle, INX234P or Dex were delivered i.v. to cynomolgus monkeys 24h prior to bleeding. Blood was shipped to ImmuNext where it was subjected to red blood cell lysis, washed once with PBS, cell pellets were then snap frozen in liquid nitrogen prior to RNA isolation. Monkeys were then euthanized and tissues perfused prior to tissue collection. Again tissues were then snap frozen in liquid nitrogen prior to RNA isolation and RNAseq analysis (Admera Health) or quantification via MS (Quintara Biosciences).

### Materials

### Test Agents and Dosage

### Antibodies

INX231P (lot #JZ-0556-017-1) is INX231 with a drug/antibody ratio (DAR) of 8.0, conjugated via full modification of the interchain disulfides. The linker/payload (P) consists of a protease sensitive linker with a budesonide derivative payload.

INX234P (lot #JZ-0556-029, JZ-0556-017) is INX234 with a drug/antibody ratio (DAR) of 8.0, conjugated via full modification of the interchain disulfides. The linker/payload (P) consists of a protease sensitive linker with a budesonide derivative payload.

### Dexamethasone

For EXPERIMENT 1, Dex (sterile injection solution, Phoenix, NDC 57319-519-05), was diluted in PBS and dosed as described via i.p. injection. For EXPERIMENT 2, Dex was administered i.v. and supplied by Bimeda MTC.

### Mice

The hVISTA KI mice were bred on site (Center for Comparative Medicine and Research at Dartmouth); C57BI/6 mice were received from Jackson Laboratories (ref #000665).

Female mice were enrolled between 9 and 15 weeks of age.

### Cynomolgus Monkeys

The study was conducted at Charles River Laboratory. Twelve cynomolgus female monkeys were enrolled, they were purpose-bred, non-naïve, with age ranging between 2- and 4-year-old.

Groups consisted in 2 animals in vehicle control treated group, 4 animals in Dex treated group and 6 animals in INX234P treated group. Animals were injected on day 1.

All the animals were bled at 24 h and remaining animals were bled again at day 8. Tissue analyses were conducted for the groups sacrificed at 24h (Dex and INX234P) and day 8 (INX234P only); 2 animals treated with Dex and 3 treated with INX234P were euthanized at 24 h and 2 animals injected with vehicle control, 2 animals treated with Dex and 3 treated with INX234P were euthanized at day 8 post injection.

### RNA Preparation and RNAseq

Cell pellets from blood and brain were resuspended in 0.4 ml of RNA lysis buffer from NucleoSpin® RNA Plus kit (Macherey-Nagel #740984). RNA was isolated following manufacturer's instructions and eluted in 30 or 40 μl H2O (RNase/DNase free). RNA concentration was assessed on Nanodrop.

RNA from bone and white adipose tissues was isolated by Admera Health. Quality control assessment and RNAseq for all samples was performed by Admera Health.

### Tissue Preparation for Mass Spectrometry

Cell pellets from various tissues were homogenized with 2 volumes of 25% methanol. All samples were diluted with corresponding plasma blank and each sample was extracted with acetonitrile containing internal standard (Verapamil). The mixture was vortexed on a shaker and subsequently centrifuged. An aliquot of the supernatant was transferred for the injection to the LC/MS/MS. Calibration standards and quality control samples were prepared by spiking the testing compound into blank cyno plasma and then processed with the unknown samples.

### Results

### Experiment 1

In this study, we evaluated the impact of INX231P and Dex on bone transcript levels in hVISTA KI (INX231P) vs. C57BI/6 mice (Dex/PBS). INX231P was dosed at 10 mg/Kg (delivering 0.2 mg/Kg of payload). Bones were isolated, bone marrow flushed and transcript expression levels measured 20h later, providing sufficient time for ADC processing and potential peak transcript expression impact. Dex was injected at 2 mg/Kg and transcript expression levels measured 2h later. INX231P effects were measured at 20h post 1 single i.p. injection at 10 mg/Kg (delivering 0.2 mg/Kg of payload). Dex effects were measured 2h post a single i.p. injection at 2 mg/Kg. Gene transcription levels were measured by RNAseq and presented as normalized counts. (n=5 mice/group; ordinary one-way ANOVA as compared to PBS-only group).

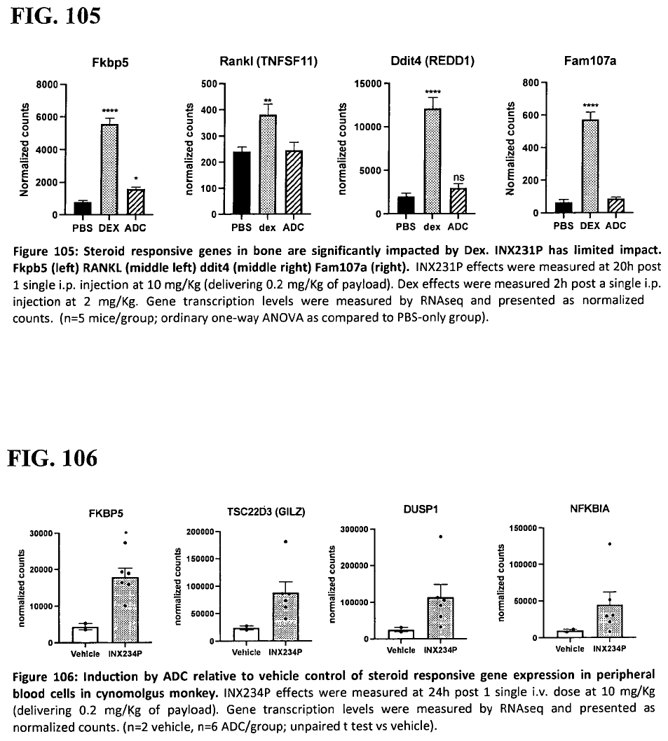

As shown in the experiment in FIG. 105, while robust FKBP5 induction was observed with Dex treatment, INX231P caused smaller changes in FKBP5 signal, likely driven by VISTA expressing immune cells. Likewise, Dex treatment led to a significant impact on a number of other bone toxicity related transcripts such as Rankl, Ddit4, Fam 107a and others (e.g., Stc2, Runx2, Errfl1—data not shown). The results indicate that the inventive ADC, INX231P, had a non-significant effect on the same transcripts. As shown in FIG. 105 steroid responsive genes in bone are significantly impacted by Dex. INX231P has limited impact. Fkpb5 (left) RANKL (middle left) ddit4 (middle right) Fam107a (right). INX231P effects were measured at 20h post 1 single i.p. injection at 10 mg/Kg (delivering 0.2 mg/Kg of payload).

### Experiment 2

### RNAseq Analyses

In this experiment in FIG. 106 we evaluated the impact of INX234P vs. Dex on transcript levels in female cynomolgus monkeys (peripheral blood leukocytes, brain, bone, white adipose) at 24h post dose (10 mg/kg-0.2 mg/kg payload). INX234P effects were measured at 24h post 1 single i.v. dose at 10 mg/Kg (delivering 0.2 mg/Kg of payload). Gene transcription levels were measured by RNAseq and presented as normalized counts. (n=2 vehicle, n=6 ADC/group; unpaired t test vs vehicle). The results show that in blood, treatment with an exemplary ADC according to the invention, INX234P, leads to a clear steroid signature with INX234P (FIG. 106). As shown therein, induction by ADC relative to vehicle control of steroid responsive gene expression in peripheral blood cells in cynomolgus monkey.

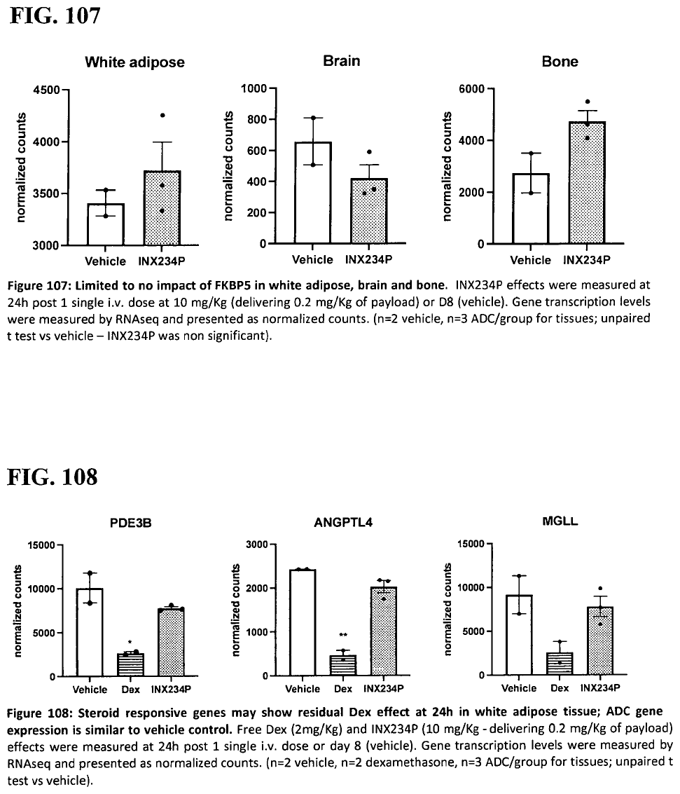

As further shown in the experiment in FIG. 107, INX234P effects in brain, white adipose and bone, on steroid related transcripts were measured at 24h post 1 single i.v. dose at 10 mg/Kg (delivering 0.2 mg/Kg of payload) or D8 (vehicle). Gene transcription levels were again measured by RNAseq and presented as normalized counts. (n=2 vehicle, n=3 ADC/group for tissues; unpaired t test vs vehicle—INX234P was non-significant). The results in FIG. 107 show that in brain, white adipose and bone, steroid related transcripts at 24h displayed limited change or were similar to control. Also, while there is some variability intra-group in these results, this is likely due to the small numbers of animals and heterogeneity among monkeys.

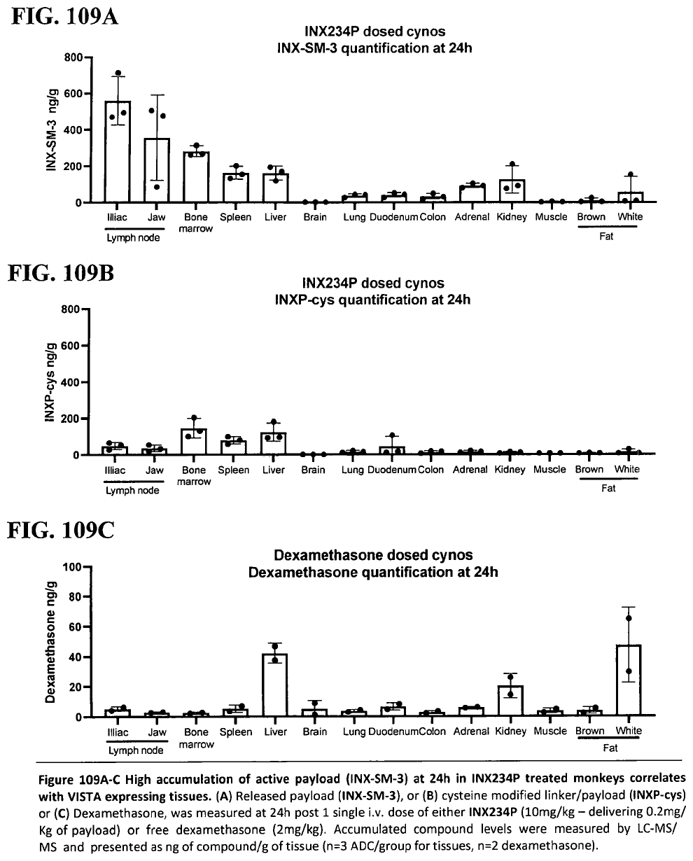

In the experiment in FIG. 108, steroid responsive genes appear to show residual Dex effect at 24h in white adipose tissue. As shown ADC gene expression is similar to vehicle control. In this experiment free Dex (2 mg/Kg) and INX234P (10 mg/Kg-delivering 0.2 mg/Kg of payload) effects were measured at 24h post 1 single i.v. dose or day 8 (vehicle). Gene transcription levels were measured by RNAseq and presented as normalized counts. (n=2 vehicle, n=2 dexamethasone, n=3 ADC/group for tissues; unpaired t test vs vehicle).

The results in FIG. 108 show that at 24h, indicate that Dex had little to no remaining impact on direct steroid responsive genes such as FKBP5 which correlates with a rapid clearance. Of note, in white adipose, PDE3B is an indirect marker of glucocorticoid action that is known to be downregulated by GC's. PDE3B was observed to be downregulated by Dex, while in ADC treated monkeys, transcript levels were similar to control (FIG. 101) (Lee R. et al. (2018) Glucocorticoid receptor and adipocyte biology. Nucl Receptor Res. 5:101373.). Other direct targets of steroid action known to be upregulated with treatment (ANGPTL4, MgII) also appeared downregulated at 24h in the Dex group (FIG. 108) (Lee R. et al. (2018) Glucocorticoid receptor and adipocyte biology. Nucl Receptor Res. 5:101373.)

We hypothesize that this could be the result of negative feedback loops following initial upregulation and provide some confirmation of where transcript expression changes occurred with glucocorticoid treatment.

### MS Analyses

In the experiment in FIG. 109A-D, the concentration of released payload (INX-SM-3) and cysteine modified linker payload (INXP-cys) was assessed in various VISTA expressing and non-VISTA expressing tissues via MS. This experiment showed the high accumulation of active payload (INX-SM-3) at 24h in INX234P treated monkeys correlates with VISTA expressing tissues. (A) Released payload (INX-SM-3), or (B) cysteine modified linker/payload (INXP-cys) or (C) Dexamethasone, was measured at 24h post 1 single i.v. dose of either INX234P (10 mg/kg—delivering 0.2 mg/Kg of payload) or free dexamethasone (2 mg/kg). Accumulated compound levels were measured by LC-MS/MS and presented as ng of compound/g of tissue (n=3 ADC/group for tissues, n=2 dexamethasone).

Particularly, at 24 h, there was a strong accumulation of released payload in VISTA expressing immune tissues and limited to no accumulation in non-VISTA expressing tissues such as colon and duodenum, fat, or brain (FIG. 109A); payloads was also detected in liver and kidney, organs in charge of filtration and excretion (FIG. 109A). There was also some accumulation of the cysteine modified linker payload which would be the expected product of catabolized ADC prior to payload release (FIG. 109B). As we expected, Dex treated monkeys had little accumulated free dexamethasone at 24h in any tissue (FIG. 109C). Residual dexamethasone did not correlate with VISTA expression, but instead was concentrated in liver, kidney and white fat. In direct contrast to Dex which was only present at low levels at 24h, robust levels of released payload (INX-SM-3) persisted at day 8 (study termination) in VISTA expressing tissues, with only low or undetectable levels in non-VISTA expressing tissues (FIG. 109D).

### Conclusions

The results of EXPERIMENT 1 showed that in mouse bone samples, Dex dosed at 2 mg/Kg induced robust levels of Fkbp5, Rankl, Ddit4, and other steroid and bone specific tox related transcripts. In contrast, INX231P dosed at 10 mg/Kg (0.2 mg/kg of payload) showed limited changes in the expression of the same transcripts as compared to the untreated group. One main toxicity associated with long term GC treatment is glucocorticoid induced osteoporosis bone damage. These data argue that steroid-anti-VISTA conjugates will have limited bone related toxicities.

The results of EXPERIMENT 2 showed that in female cynomolgus monkeys, INX234P dosed at 10 mg/Kg (or 0.2 mg/Kg of payload) had robust impacts on FKBP5 and other steroid pathway related transcripts in blood relative to vehicle control. In tissues with little to no VISTA expression, transcript levels appeared more similar to control-though results were impacted by the intrinsic variability between animals and by small sample numbers.

Dex at 2 mg/Kg had only limited impact in most tissues given that at 24 h, the small molecule should be cleared. These findings correlated with a robust intracellular accumulation at 24h of released payload from INX234P treated groups, in VISTA expressing tissues, and limited accumulation in non-VISTA expressing tissues. After 7 days (study day 8), robust levels of released payload persisted in VISTA expressing tissues such as lymph nodes (both iliac and jaw) and bone marrow. This is in stark contrast to free dexamethasone which, as expected by 24h, was detected at very low levels in non-immune tissues.

Interestingly, though Dex at 2 mg/Kg had only limited impact in most tissues given that at 24h the small molecule should be cleared, some transcripts were downregulated potentially due to negative feedback loops. This provides an indication of transcripts which should show steroid impact if present- and where we specifically see limited to no impact of our anti-VISTA steroid ADC.

Also, the results showed that the transcriptional data were strongly supported by payload accumulation data. INX234P treated monkeys showed robust levels of the released active payload of INX234P, INX-SM-3, in VISTA expressing tissues and lower levels of the full linker payload (INXP-cys). Low levels of residual dexamethasone did not correlate with VISTA expressing tissues.

Specifically, the data of EXPERIMENT 1 show that in mouse bone samples, Dex dosed at 2 mg/Kg induced robust levels of Fkbp5, along with additional bone toxicity related transcripts such as Rankl, ddit4, and Fam 107a among others. In contrast, INX231P dosed at 10 mg/Kg (0.2 mg/kg of payload) showed very limited to no changes in the expression of the same transcripts as compared to the untreated group.

In female cynomolgus monkeys, INX234P dosed at 10 mg/Kg (or 0.2 mg/Kg of payload) had robust impacts on FKBP5 and other steroid pathway related transcripts in blood relative to vehicle control. In tissues with little to no VISTA expression, transcript levels appeared more similar to control—though results were impacted by the intrinsic variability between animals and by small sample numbers.

Also, although Dex at 2 mg/Kg had only limited impact in most tissues given that at 24h the small molecule should be cleared, some transcripts were downregulated potentially due to negative feedback loops. This provides an indication of transcripts which should show steroid impact if present—and where we specifically see limited to no impact of our anti-VISTA steroid ADC.

Transcriptional data were strongly supported by payload accumulation data. INX234P treated monkeys showed robust levels of the released active payload, INX-SM-3, in VISTA expressing tissues and lower levels of the full linker payload (INXP-cys). Robust levels of released payload persisted in VISTA expressing tissues (lymph node and bone marrow) at day 8 (study termination). Low levels of residual dexamethasone at 24h did not correlate with VISTA expressing tissues.

### Example 21: Non-GLP Pharmacokinetic Study of INX234P by Intravenous Injection in Cynomolgus Monkeys

Studies were conducted at Charles River laboratories (CRL) to determine the pharmacokinetic characteristics of an exemplary ADC according to the invention, INX234P, an anti-human VISTA monoclonal antibody linked to a glucocorticoid (GC) payload, after a single injection at 15 mg/Kg in cynomolgus monkeys. Changes in immune populations, cortisol levels and intracellular/serum levels of GC payload were also measured.

In these experiments changes in white blood cells (WBC) numbers and cortisol levels were monitored over the course of the experiments. Also, released payload accumulation was quantified in both serum and blood cell pellets by mass spectrometry (MS).

### Materials and Methods

### Experimental Design

Four male cynomolgus monkeys received one dose intravenously (i.v.) of INX234P at 15 mg/Kg. The animals were then bled at the time points listed infra. Serum and blood cell pellets were collected and stored at all the timepoints.

### Test Agents and Dosage

INX234P (Abzena, HA-0853-02)
Dosing 15 mg/Kg and injected i.v.

### Cynomolgus Monkeys

Study was conducted at CRL on non-human primate, purpose bred, non-naïve from RMS Houston, Houston, TX, USA and Orient BioResource Center, Alice, TX, USA.

Four males aged between 2 and 4 year-old were enrolled in the study.

The sample collection particulars are in TABLE 8.

TABLE 8

| Sample Collection | | | | | |
|---|---|---|---|---|---|
| Group Number | Day | Time Point | Hematology | PBMC | PK |
| All animals | 1 | Predose | X | X | X |
| 1 | 1 | 0.3 hr postdose | X | X | X |
| | | 3 hr postdose | X | X | X |
| | | 6 hr postdose | X | X | X |
| | | 12 hr postdose | X | X | X |
| | | 24 hr postdose | X | X | X |
| | | 48 hr postdose | X | X | X |
| | | 72 hr postdose | X | X | X |
| | | 96 hr postdose | X | X | X |
| | Day 8 | — | X | X | X |
| | Day 15 | — | X | X | X |
| | Day 22 | — | X | X | X |
| | Day 29 | — | X | X | X |
| Unscheduled | | — | X | X | X |
| euthanasia (when | | | | | |
| possible) | | | | | |

| Method/Comments: | Venipuncture | | |
|---|---|---|---|
| Target Volume (mL)^{a}: | 0.5 | 1.3 | 1 |
| Anticoagulant: | K₃EDTA | | SST |
| Processing: | None | See infra | Serum |
| | | | See infra |

| | |
|---|---|
| Comments: | — |

X = Sample to be collected;
— = Not applicable;
hr = hour.
PK = Pharmacokinetic (for bioanalytical)
^{a}Additional samples may be obtained (e.g., due to clotting of non-serum samples) if permissible sampling frequency and volume are not exceeded.

### Hematology

The hematology specifics are in TABLE 9.

TABLE 9
Hematology Parameters

| | |
|---|---|
| Red blood cell count | White blood cell count |
| Hemoglobin concentration | Neutrophil count (absolute) |
| Hematocrit | Lymphocyte count (absolute) |
| Mean corpuscular volume | Monocyte count (absolute) |
| Red Blood Cell Distribution Width | Eosinophil count (absolute) |
| Mean corpuscular hemoglobin | Basophil count (absolute) |
| concentration | |
| Mean corpuscular hemoglobin | Large unstained cells (absolute) |
| Reticulocyte count (absolute) | Other cells (as appropriate) |
| Platelet count | |

A blood smear was prepared from each hematology sample.

### Bioanalytical Sample Processing

PK blood samples were centrifuged, the resultant serum separated, and frozen immediately over dry ice or in a freezer set to maintain −70° C. or colder.

### Bioanalytical Sample Analysis

A 96-well polystyrene microplate was coated with 0.5 μg/mL Coating Solution (Sheep anti-Human IgG, The binding site, cat #AU003.M) for 12-24 hours at 2°−8° C. After washing, blocking buffer (Pierce/Thermo Scientific, cat #37528) was added and incubated for 90 min±10 min at 350 rpm on a plate shaker at room temperature. Plates were washed and test sample added, and incubated for 2 hours±20 minutes at 350 rpm on a plate shaker at room temperature. Plates were washed and the Detection Antibody Solution at 250 ng/ml (Goat anti-Human IgG, HRP (H&L), Bethyl, cat #A80-319P) added to each well of the plate(s), incubated for 60±5 minutes at 350 rpm on a plate shaker at room temperature. Plates were washed and 100 μL of the TMB substrate solution added to the each well(s) of the plate(s) and incubated at room temperature for 15±2 minutes. 100 μL of stop solution was added to each well of the plate(s) to stop the TMB reaction.

Plates were read at 450 nm using a microplate reader (Molecular Devices SPECTRAmax) within 15 minutes stop of reaction. The test article was used for calibration curve.

### Pharmacokinetic Evaluation

Antibody half-life was determined using the PKsolver program performing a non-compartmental analysis (NCA) after intravenous bolus.

### Cortisol ELISA

Cortisol Enzyme Immunoassay kit (Arbor Assays cat #K003-H5).

Serum samples were assayed using the Cortisol ELISA kit according to vendor protocol.

### Mass Spectrometry Analyses

MS quantification was performed by Quintara Discovery on cell pellets and on serum.

### On Cell Pellets:

Briefly, cell pellets were suspended with 100 μL of 20% methanol. All samples were diluted with corresponding plasma blank and each sample was extracted with acetonitrile: methanol containing internal standard (Verapamil). The mixture was vortexed on a shaker and subsequently centrifuged. An aliquot of the supernatant was transferred for the injection to the LC/MS/MS (ExionLC AD Pump; Sciex Qtrap 6500+) . . . . Calibration standards and quality control samples were prepared by spiking the testing compound into blank cyno plasma and then processed with the unknown samples.

Normalization of the data to cell volume (ng/ml) was done by first calculating the cell numbers based on the PBL or WBC hematological count and the fixed volume for each sample processed for MS. We then converted to volume by using the arbitrary mean corpuscular volume (MCV) of 400 femtoliter per cell that is the most frequently adopted (3).

### On Serum:

Each plasma sample was extracted with acetonitrile: methanol containing internal standard (Verapamil). The mixture was vortexed on a shaker and subsequently centrifuged. An aliquot of the supernatant was transferred for the injection to the LC/MS/MS (ExionLC AD Pump; Sciex Qtrap 6500+). Calibration standards and quality control samples were prepared by spiking the testing compound into blank cyno plasma and then processed with the unknown samples.

All graphs were prepared with GraphPad (Prism).

### Results

### Pharmacokinetic Data

Animals received one dose of INX234P intravenously at a dosing of 15 mg/Kg and the PK data was analyzed using PK Solver resulting in a PK or T1/2=14 hr.

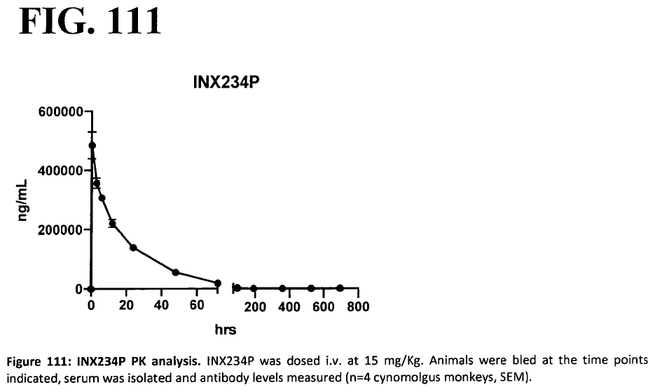

The experiment in FIG. 111 shows the INX234P PK analysis. In the experiments INX234P was dosed i.v. at 15 mg/Kg, and animals were bled at the time points indicated, serum was isolated and antibody levels measured (n=4 cynomolgus monkeys, SEM).

Additionally, the raw PK data is contained in Table 10 and Table 11 below.

TABLE 10
PK data analysis:
Table 1.1: Individual and Summary Male Cynomolgus Monkey Serum INX234P Concentrations
Following a Single IV Bolus Injection of 15 mg/kg INX234P

| | | Animal | | | | | | | |
|---|---|---|---|---|---|---|---|---|---|
| | | 1001 | 1002 | 1003 | 1004 | N | Mean | SD | % CV |
| Dose | Time | Concentration | | | | | | | |
| (mg/kg) | (hr) | (ng/mL) | | | | | | | |
| 15 | 0 | BQL | BQL | BQL | BQL | 4 | 0.00 | 0.00 | NA |
| | 0.3 | 565000 | 523000 | 528000 | 356000 | 4 | 493000 | 93000 | 18.9 |
| | 3 | 336000 | 413000 | 401000 | 325000 | 4 | 369000 | 44700 | 12.1 |
| | 6 | 303000 | 320000 | 330000 | 287000 | 4 | 310000 | 18700 | 6.05 |
| | 12 | 192000 | 235000 | 237000 | 212000 | 4 | 224000 | 28200 | 12.6 |
| | 24 | 123000 | 129000 | 148000 | 150000 | 4 | 138000 | 13500 | 9.81 |
| | 48 | 44180 | 83900 | 45300 | 70700 | 4 | 61000 | 19600 | 32.1 |
| | 72 | 11700 | 34100 | 11700 | 28100 | 4 | 21400 | 11500 | 53.6 |
| | 96 | 794 | 5720 | 3280 | 2240 | 4 | 3010 | 2080 | 69.0 |
| | 168 | BQL | BQL | BQL | BQL | 4 | 0.00 | 0.00 | NA |
| | 336 | BQL | BQL | BQL | BQL | 4 | 0.00 | 0.00 | NA |
| | 504 | BQL | BQL | BQL | BQL | 4 | 0.00 | 0.00 | NA |
| | 672 | BOL | BQL | BOL | BQL | 4 | 0.00 | 0.00 | NA |

BQL = Below the quantitation limit (LLOQ) = 20.0 ng/mL).
NA Not applicable.

TABLE 11
PK data analysis
Table 2.1: Individual INX234P Pharmacokinetic Parameters in Male Cynomolgus Monkey
Serum Following a Single 15 mg/kg IV Bolus Injection of INX234P

| Dose | | tₘₐₓ | C₀ | tₗₐₛₜ | AUCₗₐₛₜ | AUC_{(0-inf)} | Cl | V_{z} | T_{1/2} |
|---|---|---|---|---|---|---|---|---|---|
| (mg/kg) | Animal | (hr) | (ng/mL) | (hr) | (hr*ng/mL) | (hr*ng/mL) | (mL/hr/kg) | (mL/kg) | (hr) |
| 15 | 1001 | 0.3 | 599000 | 96 | 8550000 | 8570000 | 1.75 | 29.0 | 11.5 |
| | 1002 | 0.3 | 537000 | 96 | 10800000 | 11000000 | 1.37 | 32.8 | 16.6 |
| | 1003 | 0.3 | $45000 | 96 | 9880000 | 9940000 | 1.51 | 27.6 | 12.7 |
| | 1004 | 0.3 | 360000 | 96 | 9810000 | 9860000 | 1.52 | 32.4 | 14.8 |
| | N | 4 | 4 | 4 | 4 | 4 | 4 | 4 | 4 |
| | Mean | — | 310000 | — | 9770000 | 9830000 | 1.54 | 30.4 | 13.9 |
| | SD | — | 104000 | — | 933000 | 982000 | 0.159 | 2.57 | 2.28 |
| | % CV | — | 20.3 | — | 9.55 | 9.99 | 10.3 | 8.46 | 16.4 |

### Hematological Changes

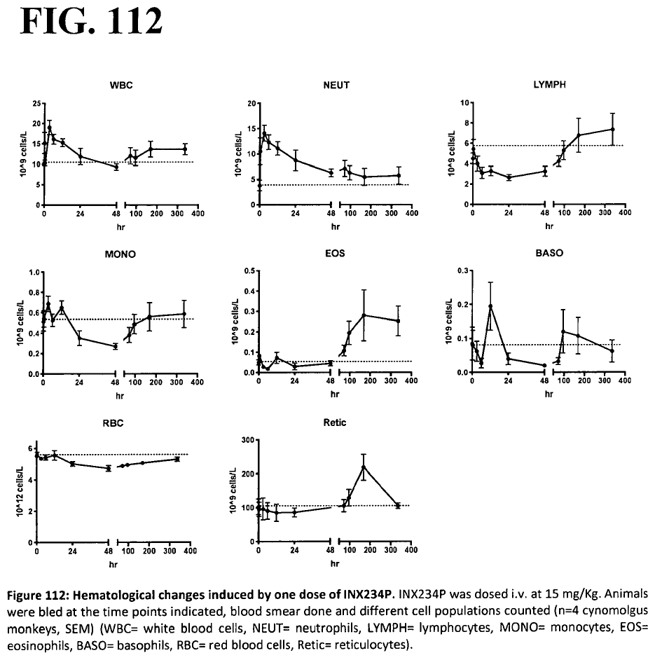

As shown from the results of the experiment in FIG. 112, all animals showed overall increases in white blood cells (WBC) with numbers going back to normal by 48 hr post injection. Lymphocytes (LYMPH) displayed a rapid decrease at 3 hr post injection. The numbers were back to normal by 96 hr. Monocytes (MONO) showed decreases by 24 hr post dosing but were also back to normal by 96 hr. These changes are consistent with the normal impact of GC which are known to cause transient leukopenia.

More particularly the results in FIG. 112 show the hematological changes induced by one dose of INX234P. INX234P was dosed i.v. at 15 mg/Kg. Animals were bled at the time points indicated, blood smear done and different cell populations counted (n=4 cynomolgus monkeys, SEM) (WBC=white blood cells, NEUT=neutrophils, LYMPH=lymphocytes, MONO=monocytes, EOS=eosinophils, BASO=basophils, RBC=red blood cells, Retic=reticulocytes).

### Cortisol Changes

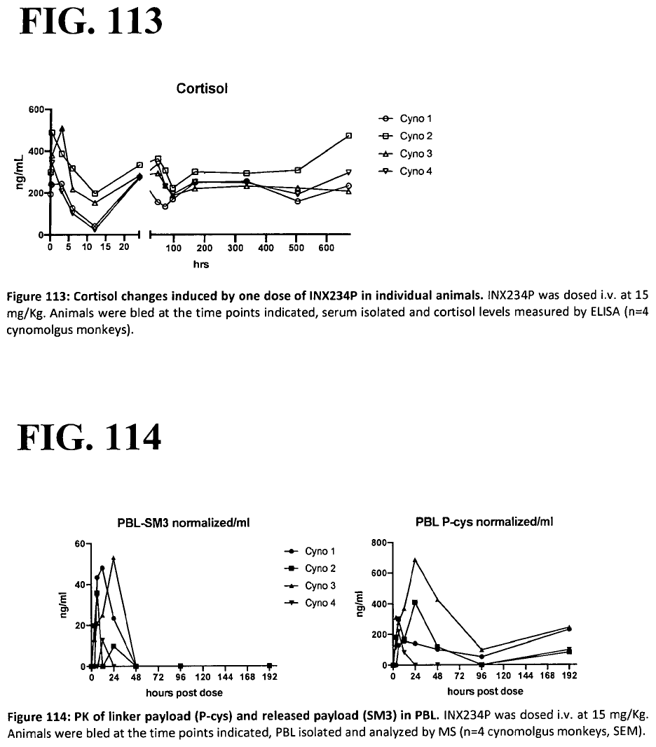

Basal cortisol levels were highly variable between animals (from 193.7 ng/ml to 298.8 ng/ml). At 20 min post i.v. injection, all the animals showed an increase in cortisol due to stress. By 12h post injection, all animals had lower to normal levels of cortisol again with high variability from animal to animal but were back to normal by 24h (the experiment in FIG. 113). Considering that that the time 0=8 AM, the drop in cortisol at ˜12h=10 PM is consistent with normal circadian rhythm in cynomolgus monkeys (Tochitani et al, 2019, “Physiological and drug-induced changes in blood levels of adrenal steroids and their precursors in cynomolgus monkeys: An application of steroid profiling by LC-MS/MS for evaluation of the adrenal toxicity”, Toxicol. Sci. Vol. 44, No. 9, 575-584).

More particularly the results in FIG. 113 show cortisol changes induced by one dose of INX234P in individual animals. INX234P was dosed i.v. at 15 mg/Kg. Animals were bled at the time points indicated, serum isolated and cortisol levels measured by ELISA (n=4 cynomolgus monkeys).

### Payload Accumulation in Serum and Peripheral Blood Leukocytes

We have shown in previous experiments that the major metabolite or released payload for INX234P is INX-SM-3. Both serum and peripheral blood leukocytes (PBL) were shipped to Quintara Discovery to run MS analyses for quantification of the linker-payload (P-cys) and released payload (SM3) over time.

The experiments in FIG. 114 show that by early timepoints, the conjugate has been internalized, and the antibody has begun to be catabolized releasing a portion of the full linker payload (INX P-cys). This accumulates in cells, and then is cleaved leading to intracellular concentrations of active released payload (INX-SM-3).

More specifically, the experiments in FIG. 114 show the PK of linker payload (P-cys) and released payload (SM3) in PBLs when INX234P was dosed i.v. at 15 mg/Kg. Animals were bled at the time points indicated, PBL isolated and analyzed by MS (n=4 cynomolgus monkeys, SEM). As the conjugate is plasma stable at 7 days (data not shown), released payload and linker payload in the plasma is representative of payload efflux from the cells.

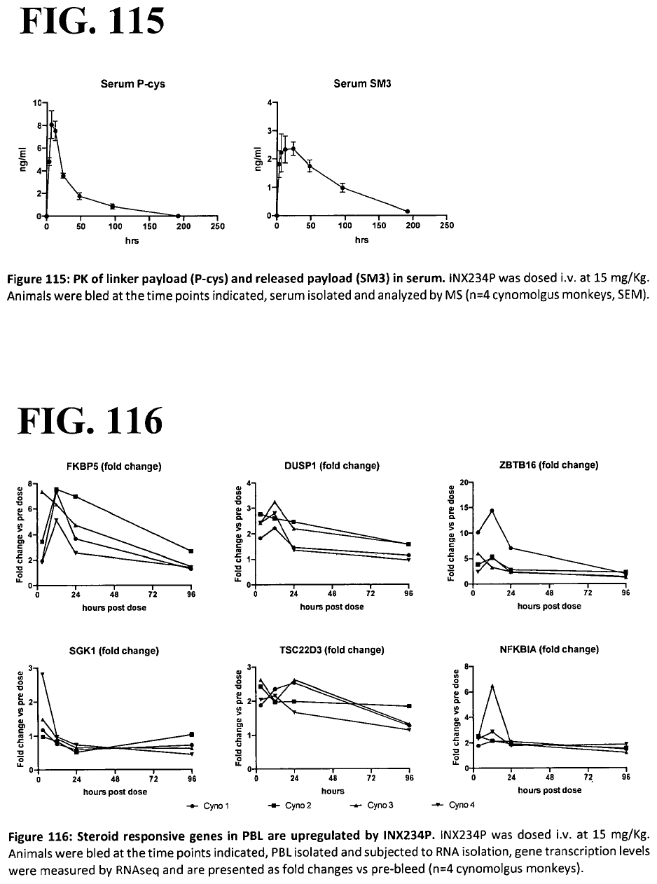

The experiments in FIG. 115 show that in serum, P-cys peaks at ˜12h while max concentration is reached at ˜24h for the released payload SM3. Of note, intracellular levels are 100-fold higher for linker-payload and 20-fold higher for released payload as compared to serum allowing efficacy but limiting the serum exposure of non-targeted tissues.

By day 8 (192 hr) post injection, both released payload and linker-payload are below detection, even though there are substantial intratissue levels of released payload in lymph nodes and bone marrow. More specifically, the experiments in FIG. 115 show the PK of linker payload (P-cys) and released payload (SM3) in serum when INX234P was dosed i.v. at 15 mg/Kg and animals were bled at the time points indicated, serum isolated and analyzed by MS (n=4 cynomolgus monkeys, SEM).

### INX234P Induced Transcriptional Changes Over Time in Peripheral Blood Leukocytes

The experiments shown in FIG. 116 show transcriptional changes in PBLs evaluated during the course of the PK study, using as baseline levels (to calculate fold changes) the blood collected before INX234P dosing (or pre bleed).

As shown in FIG. 116, despite variability between animals, steroid transcriptional targets such as FKBP5, DUSP1, ZBTB16, TSC22D3 and NFKBIA show rapid increases in transcription as early as 3 hrs post dosing, with a peak around 12 hr for FKBP5, DUSP1 and ZBTB16. At 24 hr most transcriptional targets are still upregulated.

As shown therein, at 96 hr, transcriptional levels for FKBP5, ZBTB16, and NKBIA were still 2-fold above baseline, while DUSP1, SGK1 and TSC22D3 were back to baseline.

More specifically the experiments in FIG. 116, show that steroid responsive genes in PBLs are upregulated by INX234P when INX234P was dosed i.v. at 15 mg/Kg. Animals were bled at the time points indicated, PBL isolated and subjected to RNA isolation, gene transcription levels were measured by RNAseq and are presented as fold changes vs pre-bleed (n=4 cynomolgus monkeys).

### Conclusion

The data in this example show that:
INX234P has a half-life (T_{1/2}) of 14 hr.
INX234P induces limited and transient cellular changes in peripheral blood, as expected for GC. The antibody component does not appear to add toxicity.
No consistent changes in cortisol levels were observed following INX234P dosing aside from those expected due to circadian rhythms.
Released linker payload, and subsequent released payload accumulation in blood cells occurs within a few hours, and results in intracellular concentrations in blood cells at levels 20- to 100-fold higher than in the serum.
No detectable levels in PBLs at day 8 while released payload is still persistent in the bone marrow and lymph node tissues possibly due to cell turnover.
INX234P induces transcriptional changes in PBL as early as 3 hr post dosing and for some targets changes still detectable at 96 hr demonstrating that the delivered payload is functionally active with extended pharmacodynamic range.
   1—Vermeer et al. (2003) Glucocorticoid-induced increase in lymphocytic FKBP51 messenger ribonucleic acid expression: a potential marker for glucocorticoid sensitivity, potency, and bioavailability. J Clin Endocrinol Metab. January; 88 (1): 277-84
   2—McPherson M J, et al. (2017) Glucocorticoid receptor agonist and immunoconjugates thereof, U.S. Ser. No. 15/611,037
   3-Khoo et al, (2002) Intracellular Accumulation of Human Immunodeficiency Virus Protease Inhibitors, Antimicrob. Agents Chemother. 2002 October; 46 (10): 3228-3235.
   4-Tochitani et al, 2019, J. Physiological and drug-induced changes in blood levels of adrenal steroids and their precursors in cynomolgus monkeys: An application of steroid profiling by LC-MS/MS for evaluation of the adrenal toxicity, Toxicol. Sci. Vol. 44, No. 9, 575-584

### Example 22: Identification and Quantification of Released Payload for INX Antibody Glucocorticoid Conjugates in Human Peripheral Blood Mononuclear Cells

In this example experiments were conducted showing that exemplary anti-VISTA glucocorticoid conjugates according to the invention are rapidly internalized into VISTA expressing cells. Antibody is catabolized releasing cysteine modified full linker payload. Linker payload is also cleaved releasing active payload. Given the diversity of proteases in the cell, the released payload must be confirmed empirically. In this study, the released payload from internalized novel antibody glucocorticoid conjugates in human peripheral blood mononuclear cells (PBMC) was identified and the abundance quantified via mass spectrometry (MS) for three different glucocorticoid linker payloads.

The objective of the present studies was to identify and quantify the released payload of novel glucocorticoid conjugates according to the invention in human PBMCs.

### Materials and Methods

### Experiment Design

In all the following experiments, human PBMC, isolated from 1 healthy donor per study, were treated with exemplary anti-VISTA glucocorticoid conjugates according to the invention to allow identification and quantification of the released payload.

In pilot experiments, we showed that 4 hours (h) of incubation with anti-VISTA glucocorticoid conjugates, led to robust increases in FKBP5 in human PBMC. This is a direct impact of glucocorticoid activity.

In the present experiments, after incubating PBMC with glucocorticoid (GC) antibody drug conjugates (ADC) for 4h, cells were harvested and assessed for both FKBP5 transcription induction via quantitative real time PCR (qPCR) as well as assessed via mass spectrometry (MS) for released payload identification and quantification.

### Reagents

### Test ADCs

INX201J (Abzena, lot #JZ-0556-025-1), is INX201 conjugated to linker/payload via full modification of the interchain disulfides with a drug/antibody ratio (DAR) of 8.0. The linker/payload (J) is based on a patent reported linker/payload (U.S. Ser. No. 15/611,037). It consists of a negatively charged protease sensitive linker with a budesonide analog payload (INX J-2).
INX231J (Abzena, lot #JZ-0556-013-1) is INX231 conjugated with a DAR of 8.0. The linker/payload (J) is based on a patent reported linker/payload (U.S. Ser. No. 15/611,037). It consists of a negatively charged protease sensitive linker with a budesonide analog payload (INX J-2).
INX231P (Abzena, lot #JZ-0556-017-1) is INX231 with a DAR of 8.0, conjugated via full modification of the interchain disulfides. The linker/payload (INX P) consists of a negatively charged protease sensitive linker with a budesonide analog payload (INX-SM-3).
INX231V (Abzena, lot #PP-0920-014-2) is INX231 with a DAR of 7.8, conjugated via modification of the interchain disulfides. The linker/payload (INX V) consists of a negatively charged protease sensitive linker with a budesonide analog payload (INX-SM-32).
INX234A11 (Abzena, lot #RJS-1054-001) is the INX234 antibody with a DAR of 8.0, conjugated via full modification of the interchain disulfides. The linker/payload (INX A11) consists of a negatively charged protease sensitive linker (Asn/gly) with a budesonide analog payload (INX-SM-32).
INX234A3 (Abzena, lot #PP-0924-023-1) is the INX234 antibody with a DAR of 8.0, conjugated via full modification of the interchain disulfides. The linker/payload (INX A3) consists of a positively charged protease sensitive linker with a budesonide analog payload (INX-SM-32).
INX234V (Abzena, lot #RJS-1054-003) is the INX234 antibody with a DAR of 8.0, conjugated via full modification of the interchain disulfides. The linker/payload (INX V) consists of a negatively charged protease sensitive linker with a budesonide analog payload (INX-SM-32).
INX231V (Abzena, lot #PP-0920-014-2) is INX231 with a DAR of 7.8, conjugated via modification of the interchain disulfides. The linker/payload (INX V) consists of a negatively charged protease sensitive linker with a budesonide analog payload (INX-SM-32).
INX231A7 (Abzena, lot #RJS-1054-007-001) is the INX231 antibody with a DAR of 7.8, conjugated via modification of the interchain disulfides. The linker/payload (INX A7) consists of a negatively charged protease sensitive linker with a budesonide analog payload (INX-SM-32) that is phosphorylated.
INX231A12 (Abzena, lot #RJS-1054-007-002) is the INX231 antibody with a DAR of 6.99, conjugated via modification of the interchain disulfides. The linker/payload (INX A12) consists of a negatively charged protease sensitive linker with a fluocinolone acetonide analog payload (INX-SM-25) that is phosphorylated.
INX231A23 (Abzena, lot #RJS-1054-006-001) is the INX231 antibody with a DAR of 7.34, conjugated via modification of the interchain disulfides. The linker/payload (INX A23) consists of a negatively charged protease sensitive linker with a fluocinolone acetonide analog payload (INX-SM-25).
INX234A5 (Abzena, lot #RJS-1054-002) is the INX234 antibody with a DAR of 7.9, conjugated via modification of the interchain disulfides. The linker/payload (INX A5) consists of a negatively charged protease sensitive linker with a budesonide analog payload (INX-SM-44).
INX234A4 (Abzena, lot #PP-0924-023-2) is the INX234 antibody with a DAR of 7.9, conjugated via modification of the interchain disulfides. The linker/payload (INX A4) consists of a negatively charged protease sensitive linker with a budesonide analog payload (INX-SM-43).
INX231J (Abzena, lot #JZ-0556-013-1) is the INX231 antibody with a DAR of 8.0, conjugated via modification of the interchain disulfides. The linker/payload (INX J) is based on a patent reported linker/payload (U.S. Ser. No. 15/611,037). It consists of a negatively charged protease sensitive linker with a budesonide analog payload (INX J-2).
INX231P (Abzena, lot #JZ-0556-017-1) is INX231 with a DAR of 8.0, conjugated via full modification of the interchain disulfides. The linker/payload (INX P) consists of a negatively charged protease sensitive linker with a budesonide analog payload (INX-SM-3).
INX234P (Abzena, lot #HA-0853-02) is INX234 with a DAR of 8.0, conjugated via full modification of the interchain disulfides. The linker/payload (INX P) consists of a negatively charged protease sensitive linker with a budesonide analog payload (INX-SM-3).
INX234J (Abzena, lot #JZ-0556-013-2) is the INX234 antibody with a DAR of 8.0, conjugated via full modification of the interchain disulfides. The linker/payload (INX J) is based on a patent reported linker/payload (U.S. Ser. No. 15/611,037). It consists of a negatively charged protease sensitive linker with a budesonide analog payload (INX J2).
INX231S (Abzena, lot #PP-0924-014-1) is the INX231 antibody with a DAR of 6.9, conjugated via modification of the interchain disulfides. The linker/payload (INX S) consists of a negatively charged protease sensitive linker with a fluocinolone acetonide analog payload (INX-SM-24).

### Cell Culture Media

RPMI 1640 without L-glutamine (VWR cat #16750-084)
Penicillin/Streptomycin/Glutamine (ThermoFisher cat #10378016)
1M Hepes (Gibco cat #15630-080)
Human AB serum (Valley Biomedical cat #HP1022HI)

### Other Reagents.

Ficoll-Paque Plus (GE Healthcare cat #17-1440-03)
Histopaque 1077 (Sigma Aldrich)

### PBMC Preparation

Human PBMC were isolated under sterile conditions from apheresis cones obtained from the Blood Donor Program at the Dartmouth Hitchcock Medical Center from deidentified healthy human donors.

The blood was transferred to a 50 ml Falcon tube and diluted with PBS to 30 ml. 13 ml of Histopaque 1077 was slowly layered under the blood, and tubes were centrifuged at 850×g for 20 min at RT with mild acceleration and no brake.

Mononuclear cells were collected from the plasma/Ficoll interface, resuspended in 50 ml of PBS and centrifuged at 300×g for 5 min. Cells were resuspended in PBS and counted.

### Assay Protocol for Both Metabolite ID and Quantitation

Isolated PBMCs were resuspended in RPMI 1640 containing 10% human AB serum, 10 mM Hepes, 1× Penicillin/Streptomycin/L-Glutamine (assay media).

PBMCs from 1 donor were washed 2× in PBS, and then resuspended at 1×10\hat{ }7/mL in media. 1 mL was transferred to each well of the 6 well plates used.

ADC or media alone was added to each well of the 6 well plates used for a final concentration of 1 mM payload with a total volume per well of 2 mL after addition of drug. The plates were placed at 37° C. for 4h.

After 4 h, the media with cells was harvested and combined from all wells of a given treatment.

1 mL of the cell suspension was pelleted, media removed, and the pellet resuspended in RNA lysis buffer to allow FKBP5 analysis.

Remaining cells for each treatment were dived into aliquots representing 50 million cells per aliquot (for metabolite quantification only one aliquot per condition was generated).

Cells were pelleted for 4 min at 450 rcf.

Media was removed from each cell pellet with the media then set aside at −80C until it could be assessed for the concentration of effluxed payload. Any additional residual media was removed from the tube leaving dry cell pellets which were stored at −80° C. for MS analysis.

As an additional control, the stock drug (2 mM) was diluted to the final concentration (1 mM) with media and aliquots set aside to confirm the lack of free payload in the media at the 0 timepoint.

### MS Analysis

MS analysis was performed by Quintara Discovery.

### For Metabolite ID

Briefly, pre-chilled 70% ACN (extraction buffer) was added to the PBMC pellets for extraction. The pellets were suspended into the extraction buffer and incubated in the ice bath. The samples were then centrifuged, and supernatant was collected from each sample. The supernatant was dried by the Speed Vac and reconstituted with 50% MeOH. 15 mL was injected onto the LCMS (Luna C18 (2) column, Thermo Scientific Q Exactive Hybrid Quadrupole Orbitrap) and resulting masses assessed.

### For Quantification

Cell samples were suspended with 100 μL 20% methanol. Each sample was extracted with acetonitrile: methanol (95:5, v/v) containing internal standard (Verapamil). The mixture was vortexed on a shaker and subsequently centrifuged. An aliquot of the supernatant was transferred for the injection to the LC/MS/MS (Sciex Qtrap 6500+). Calibration standards and quality control samples were prepared by spiking the testing compound into untreated human PBMC and then processed with the unknown samples.

### RNA Preparation and Real Time PCR

Cell pellets were resuspended in 0.4 ml of RNA lysis buffer from NucleoSpin® RNA Plus kit (Macherey-Nagel #740984). RNA was isolated following manufacturer's instructions and eluted in 30 or 40 ml H2O (RNase/DNase free). RNA concentration was assessed on Nanodrop.

Reverse transcription was done using Taqman reverse transcription reagents (#N8080234) and following manufacturer's instructions.

Quantitative Real-Time PCR was done using Taqman master mix 2× kit (#4369016) and run on a QuantStudio3 from Applied Biosystem. Primers used:
i. TaqMan Gene Expression Assay (FAM-MGB); Assay ID: Hs01561006_m1 (FKBP5)
ii. TaqMan Gene Expression Assay (FAM-MGB); Assay ID: Hs01922876_u1 (GapDH) Ct data were converted to ΔCt and ΔΔCt or Log 2 fold changes compared to untreated control.

### Results

### Experiment 1

### INX-SM-J2, INX-SM-3, and INX-SM-32 are the Major Metabolites of INX201J, INX231P, and INX231V Respectively.

In this experiment, we identified the major metabolites from incubation of INX201J, INX231P, and INX231V after 4 hrs of in vitro incubation with human PBMC (single donor).

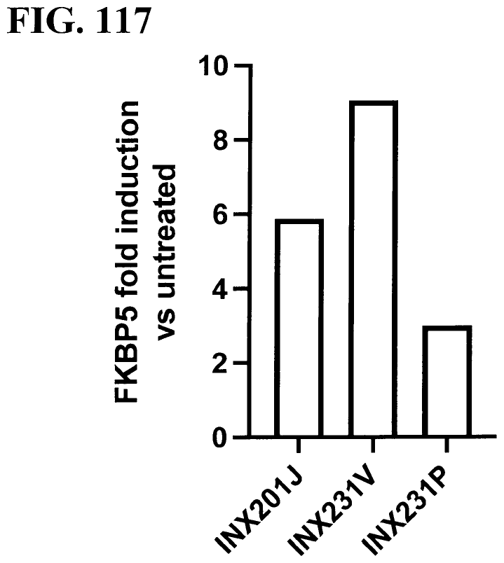

As shown in FIG. 117, incubation with all conjugates lead to a robust increase in FKBP5 transcription. MS analysis confirmed that the major metabolite for any of the conjugates was their respective predicted released payloads, for INX201J this corresponded to INX-SM-J2, and for INX231P and INX231V, INX-SM-3 and INX-SM-32 respectively. These released payloads in their purified form have each been shown to be active in other experiments results reported herein and to elicit similar potency. Lower levels of uncleaved cysteine modified linker payload, representing catabolized antibody, were also present for each of the three conjugates.

The data in TABLE 12 provides the approximate abundance of released payload, uncleaved cysteine modified linker payload and compound in media representing efflux of released compound. Of note, the abundance of released payload from INX231V vastly exceeds that of the released payload from INX231P and INX201J.

The difference in released payload between INX231P and INX201J is confounded by the fact that INX201 is internalized much more rapidly both in vivo and in vitro than INX231 (data not shown), leading to additional accumulation of INX201 and thereby released INX J payload over INX P payload. Even when compared to a conjugate of an antibody that internalizes more rapidly, the level of INX V released payload still substantially exceeds that of the corresponding INX J released payload.

TABLE 12

| | | Estimated | | Estimated | Amount of |
|---|---|---|---|---|---|
| | | Amount of | | Amount of | effluxed |
| | | Linker | | Released | released |
| | | Payload in | | Payload in | payload in |
| | Linker | 5 × 10⁷ | Released | 5 × 10⁷ | media |
| ADC | Payload | Cells (ng) | payload | Cells (ng) | (ng/mL) |
| INX201J | INXJ- | 1.8 | INX-J2 | 2.6 | 1.7 |
| | Cys | | | | |
| INX231P | INXP- | ND | INX-SM- | 1.2 | BQL |
| | Cys | | 3 | | |
| INX231V | INXV- | 3.3 | INX-SM- | 205 | 20.9 |
| | Cys | | 32 | | |

As shown in TABLE 12, there was substantial release of INX V payload vs INX P or INX J. Estimated intracellular payload abundance at 4h for human PBMC incubated with 1 mM conjugated payload (˜20 mg/mL ADC) and quantified via MS analysis. The amount of effluxed payload present in the media at 4h is also given. The level in the media is quantified rather than estimated as it was assessed in parallel with the samples from another experiment (n=1 donor (ND—Not detectable (Estimated LLOQ=0.1 ng); BQL—below quantitation limit (<0.5 ng/ml)).

### Experiment 2

INX V Released Payload Present at Higher Levels than INX P or INX J Released Payloads.

In this study, we confirmed the abundance of released payload after 4h incubation of INX231J, INX231P, and INX231V with human PBMCs. TABLE 13 provides the abundance of released payload, cysteine modified linker payload and compound in media representing efflux of released compound. It also includes as a control, the level of released payload detected when the ADC stock solutions were diluted with media (no cells present) and submitted for MS analysis.

The results of this study confirms and quantifies the dramatic 235x enhancement (ng/ng) of INX V released payload over INX P and even greater over the INX J released payload. This substantial enhancement in release was not expected based on the structural differences among the three linker payloads, and was not the result of antibody or linker contribution as the same antibody (INX231) and gly glu linker were used for all three conjugates.

Despite the fact that the antibody is the same for INX P and INX J conjugates, INX P released payload is ˜3× the amount of the INX J released payload at 4 hrs. These results further corroborate the potency benefit observed in the LPS stimulated PBMC assay shown in a previous example.

TABLE 13

| | | | | | Amount of |
|---|---|---|---|---|---|
| | | Amount of | | Amount of | effluxed |
| | | Linker | | Released | released |
| | | Payload in | | Payload | payload in |
| | Linker | 5 × 10⁷ | Released | in 5 × 10⁷ | media |
| ADC | Payload | cells (ng) | Payload | cells (ng) | (ng/mL) |
| INX231J | INXJ- | 0.5 | INX-J2 | 0.2 | 1.2 |
| | Cys | | | | |
| INX231P | INXP- | 0.4 | INX-SM- | 0.7 | 0.5 |
| | Cys | | 3 | | |
| INX231V | INXV- | 3.1 | INX-SM- | 165 | 22.8 |
| | Cys | | 32 | | |

The data in TABLE 13 show that the levels of INX231V released payload exceed those of INX231J or INX231P. Quantified abundance of intracellular payload at 4h for human PBMC incubated with 1 mM conjugated payload (˜20 mg/mL ADC) and quantified via MS analysis is shown therein. The amount of effluxed payload present in the media at 4h is also given. Released payload levels detected in the stock solution was 0.8 ng/ml for INX231J or below the limit of quantitation-<0.5 ng/ml (INX231V/INX231P). Cysteine quenched payload levels in the stock solution were 0.9 ng/mL for INX231P, or below the limit of quantitation, <0.5 ng/ml (INX231V/INX231P).

### Experiment 3

Quantification of Released Payload from INX A11, INX A3, INX V, INX A7, INX A12, INX A23, INX A5, INX A4, INX P, INX S and INX J Anti-VISTA Conjugate Incubated with Human PBMC

In this study, we confirmed the abundance of released payload from a series of novel glucocorticoid conjugates after 4h incubation with human PBMC (1 donor). TABLE 14 below provides the abundance of released payload and compound in media representing efflux of released compound. This study also included as a negative control, the level of released payload detected when the ADC stock solutions were diluted with media (no cells present) and submitted for MS analysis. All amounts are given as ng/ml. In two cases (INX234P and INX234J) a fraction of the material was lost due to technical changed prior to analysis, and so amount of intracellular material is normalized to account for the loss of material.

This study confirms and quantifies the dramatic enhancement (ng/ng) of ImmuNext novel linker payloads over the INX J literature comparator. Of note, when the antibody backbone is held constant (e.g., INX231) and is conjugated to either INX J or an ImmuNext linker payload, greater amounts of released ImmuNext payloads are detected intracellularly. In the one case where they are more similar (e.g., INX234P vs INX234J), the INX234P was detected at higher levels intracellularly (˜1.5×), and yet in contrast to INX J had no detectable effluxed payload in the media. This is important as in vivo, effluxed payload may result in shortened retention by targeted cell types and non-specific uptake by other cells.

TABLE 14

| | | Amount of | |
|---|---|---|---|
| | | Released | |
| | | Payload in | Amount of effluxed |
| | Released | 5 × 10⁷ cells | released payload in |
| ADC | Payload | (ng/ml) | media (ng/mL) |
| INX234A11 | INX-SM-32 | 50.5 | BQL (<0.2 ng/mL) |
| INX234A3 | INX-SM-32 | 21.4 | BQL (<0.2 ng/mL) |
| INX234V | INX-SM-32 | 25.2 | BQL (<0.2 ng/mL) |
| INX231V | INX-SM-32 | 343.0 | 9.4 |
| INX231A7 | INX-SM-32 | 9.1 (INX-SM-32); | BQL (<0.2 ng/mL) (INX-SM-32); |
| | (dephosph); | 1.1 (INX-SM-38) | BQL (<0.2 ng/mL) (INX-SM-38) |
| | INX-SM-38 | | |
| | (phosph) | | |
| INX231A12 | INX-SM-25; | 6.7 (INX-SM-25); | BQL (<0.2 ng/mL)(INX-SM-25); |
| | (dephosph) | 1.4 (INX-SM-21) | BQL (<0.5 ng/mL) (INX-SM-21) |
| | INX-SM-21 | | |
| | (phosph) | | |
| INX231A23 | INX-SM-25 | 12.4 | BQL (<0.2 ng/mL) |
| INX234A5 | INX-SM-44 | 34.1 | BQL (<0.5 ng/mL) |
| INX234A4 | INX-SM-43 | 38.1 | BQL (<0.2 ng/mL) |
| INX231S | INX-SM-24 | 14.8 | 0.3 |
| INX231P | INX-SM-3 | 1.3 | BQL (<0.2 ng/mL) |
| INX234P | INX-SM-3 | 2.9 | BQL (<0.2 ng/mL) |
| INX234J | INX-J2 | 2.0 | 0.2 |
| INX231J | INX-J2 | 0.5 | BQL (<0.2 ng/mL) |

The data in TABLE 14 shows that the levels of novel linker payload released payload exceed those of INX J comparators. The quantified abundance of intracellular payload at 4h for human PBMC incubated with 500 nM conjugated payload (˜10 mg/mL ADC) and quantified via MS analysis is shown in the TABLE. The amount of effluxed payload present in the media at 4h is also given. Released payload levels detected in the stock solution were all BQL (below the limit of quantitation <0.5 ng/ml).

### Conclusions

Anti-VISTA conjugates with each of the specific linker payloads (INX J, INX V, and INX P) are each cleaved releasing the theorized released payload. Additional alternative cleavage products are not present at detectable levels.
Some residual full cysteine modified linker payload is still present.
Released payload from INX V conjugate, and other novel glucocorticoid linker payloads is more abundant than the corresponding released payloads for INX J.

Particularly, in the experiments in this example we identified the major metabolite of INX201J, INX231P, and INX231V as being the theorized released payload INX-J2, INX-SM-3, and INX-SM-32 respectively.

Surprisingly, the released payload for INX231V was present at substantially greater amounts than the payload for INX P and INX J after 4h of incubation. It was estimated in an initial study to be >75× abundant (ng/ng) relative to the payloads for INX P and INX J conjugates. In a second study, it was confirmed to be present at >230× abundance (ng/ng) relative to INX P and even greater for the INX J released payloads.

INX P conjugates also demonstrated enhanced accumulation (˜3×) of released payload over the INX J conjugates when the same antibody was used for each conjugate.

This increased abundance has substantial implications for both potency and PD. Additional studies using a delayed lipopolysaccharide (LPS) assay disclosed in a previous example have proved INX V conjugates to be substantially more potent in their conjugated form than INX P, which is more potent than the INX J conjugates of the same antibody, even though the released payloads for the three are similarly potent in their free form. The large intracellular depot of released payload observed here in vitro may also contribute directly to the multi-week sustained effect observed in vivo even though the ADCs have a short serum half-life of only hours.

All additional novel glucocorticoid conjugates we tested also showed enhanced to dramatically enhanced intracellular accumulation over INX J conjugated to the same antibody backbone, with little released payload effluxed into the media. This data is strongly supportive of the enhanced in vitro potency and long PD of the inventive ADCs relative to the anticipated potency given the potency of the free payload form and the very short PD of glucocorticoid small molecules in general.

### Example 23: IBD or Colitis Study

The Dextran sodium sulfate colitis murine model (DSS) model is commonly used to assess potential IBD or colitis therapeutics. (See, Eichele et al., “Dextran sodium sulfate colitis murine model: An indispensable tool for advancing our understanding of inflammatory bowel diseases pathogenesis”, World J Gasteroenterology, 2017 Sep. 7; 23 (33): 6016-6029″). Accordingly, this animal model was used to preliminarily assess the efficacy of an ADC according to the invention for treatment of colitis or IBD.

Also as is generally known IBD and colitis are chronic, conditions which are difficult to effectively treat and manage, and which, if ineffectively treated may result in sepsis and death. Currently the primary means of IBD or colitis disease management involves chronic steroid administration. However, unfortunately this potentially can cause toxicity, e.g., because of effects of the steroid on non-target (e.g., epithelial cells) and/or prolonged immunosuppression.

In this preliminary experiment one animal group was administered a Dex ADC according to the invention (INX234P) at a steroid dose of 0.2 mpk every other day, a second positive control animal group was administered free Dex steroid at a steroid dose of 2 mpk every day, and the third negative control animal group was untreated. There were 10 animals per group. ADC or Dex treatment was initiated when animals started showing weight loss (day 7) (DSS started on day 0). The experiment terminated on day 13 when one group (dex treated) reached maximum allowed weight loss.

The results (preliminary, not shown) suggest that the ADC showed efficacy compared to the untreated control. Also, the results suggest that the ADC did not elicit the same toxicity observed in the free steroid treated animal. With respect thereto, it has been reported that dexamethasone causes toxicity in this IBD model; see van Meeteren M E, Meijssen M A C, Zijlstra F J. “The effect of dexamethasone treatment on murine colitis”, Scand J Gastroenterol 2000; 35:517-521; and Ocon et al., “The glucocorticoid budesonide has protective and deleterious effects in experimental colitis in mice”, Biochemical Pharmacology 116 (2016) 73-88).

While these results are preliminary, they suggest that the subject ADCs may be useful in treating colitis or IBD indications. Also, they suggest that the subject ADCs may be preferred over existing free steroid therapies for treating these chronic diseases as they may alleviate the toxicity which may occur during prolonged free steroid therapies. This is further corroboirated in Example 25.

### Example 24: Impact of Antibody Drug Conjugate INX234P and INX234V on T Cell Expansion and Survival in a Xeno-GvHD Model-2 Studies

Humanized mouse models of xenogeneic Graft versus Host Disease (xeno-GvHD) allow the study of immunomodulatory compounds specific to human drug targets in vivo. These are based on immunodeficient strains of mice injected with peripheral blood mononuclear cells (PBMCs) from human. The NOD-scid IL-2Rγ null (NSG) strain lack mature T cells, B cells and natural killer cells, and are amendable to xenogeneic GvHD studies.

In the NSG model of xeno-GvHD, donor human T cells are transferred intravenously (i.v.) and expand robustly over time in recipient mice and effect anti-host cell reactivity leading to cutaneous tissue infiltration. The mice lose weight and if left untreated will succumb to GvHD. The timeframe of disease progression can range from 3 to 6 weeks. The time of disease occurrence and progression can be accelerated by irradiating the mice with 2.5-3Gy prior to transfer of the human PBMCs. In this case, the disease initiates after approximately 1-2 weeks and mice will succumb by the 2-3-week mark.

Briefly, mice were first injected with 2.5×10⁶ human PBMCs along with a dose of control human IgG1, INX234, or INX234P (EXPERIMENT 1) or INX234V (EXPERIMENT 2). Disease progression was monitored by regularly weighing mice as well as measuring human T cell expansion by absolute leukocyte count (ALC) on peripheral blood. The objective of the studies was to evaluate the ability of human VISTA antibody INX234 conjugated to 2 different GC payloads to impact progression of xeno-GvHD.

### Materials and Methods

### Study Design

In these experiments, after the first dose of antibodies or ADC, injected i.v. with the donor cells, treatment was done via intraperitoneal injection (i.p.), once a week, at 10 mg/Kg till week 34.

Blood was collected only once on day 21 for EXPERIMENT 1. Blood was collected once a week, starting on day 15 for EXPERIMENT 2.

### Test Agents and Dosage

INX234 (ATUM, lot #72931.2.a) is a humanized anti-human VISTA antibody on a human IgG1/kappa backbone with L234A/L235A/E269R/K322A silencing mutations in the Fc region.
INX234P (Abzena, lot #JZ-0556-017-2) is INX234 with a drug/antibody ratio (DAR) of 8.0, conjugated via full modification of the interchain disulfides. The linker/payload (INX P) consists of a negatively charged protease sensitive linker with a budesonide analog payload (INX-SM-3).
INX234V (Abzena, lot #RJS-1054-003) is the INX234 antibody with a DAR of 7.9, conjugated via modification of the interchain disulfides. The linker/payload (INX V) consists of a negatively charged protease sensitive linker with a budesonide analog payload (INX-SM-32).
huIgG1si (Aragen, lot #BP-2211-018-6) is an anti-RSV monoclonal Ab on a human IgG1/kappa backbone with E269R/K322A silencing mutations in the Fc region.

All antibodies were diluted in PBS and injected i.p. in a volume of 0.2 ml to deliver a dose of 10 mg/Kg.

In EXPERIMENT 2, animals were treated with INX234V till day 27 and then with INX234P during the rest of the experiment.

### Mice

8-week-old NSG mice were purchased from the Jackson Laboratory (NOD.Cg-Prkdc^{scid} II2rg^{tm1WjI} J Stock No: 005557) and housed in specific pathogen free conditions at the Dartmouth-Hitchcock Medical Center (DHMC). Mice were tattooed prior to the initiation of the experiment.

Antibody

Human CD25 FITC
Human CD19 PE
Mouse CD45 PerCP cy5.5
Human CD45RO PECy7
Huma CD4 APC-cy7
Human CD3 BV421
Human CD45 BV510
Mouse Fc block

For EXPERIMENT 1, mice were irradiated at 2.5 Gy, 7 days before PBMC i.v. transfer.

For EXPERIMENT 2, mice were not irradiated.

### Blood Draw and Immunostaining

Peripheral blood was harvested from the retro-orbital cavity using a glass Pasteur pipette that was first rinsed with heparin to prevent coagulation.

The 1-wash protocol described below allows for absolute blood cell count.

10 ml of antibody cocktail (see on the right) was directly added to 100 ml of blood. After 30 min incubation at room temperature (RT), 600 ml BD FACS lysis buffer was added to the sample. After 30 min incubation at RT, samples were spun at 550 rcf for 5 min, washed once in PBS and then resuspended in a fixed volume of PBS. The whole sample was run on a MacsQuant flow cytometer to obtain an absolute cell number.

### Peripheral Blood Mononuclear Cell Isolation

Human PBMC were isolated under sterile conditions from apheresis cones obtained from the Blood Donor Program at the DHMC from deidentified healthy human donors. The blood was transferred to a 50 ml Falcon tube and diluted with PBS to 30 ml. 13 ml of Histopaque 1077 (Sigma Aldrich) was slowly layered under the blood, and tubes were centrifuged at 850 rcf for 20 min at RT with mild acceleration and no brake.

Mononuclear cells were then collected from the plasma/Ficoll interface, resuspended in 50 ml of PBS and centrifuged at 300×g for 5 min. Cells were resuspended in PBS and counted.

### Disease Monitoring

Mice were weighed 3 times a week and euthanized once weight dropped below 75% of their initial weight as allowed by our IACUC organization.

### Results

### Experiment 1

### INX234P Treatment Prevents Human PBMC Expansion

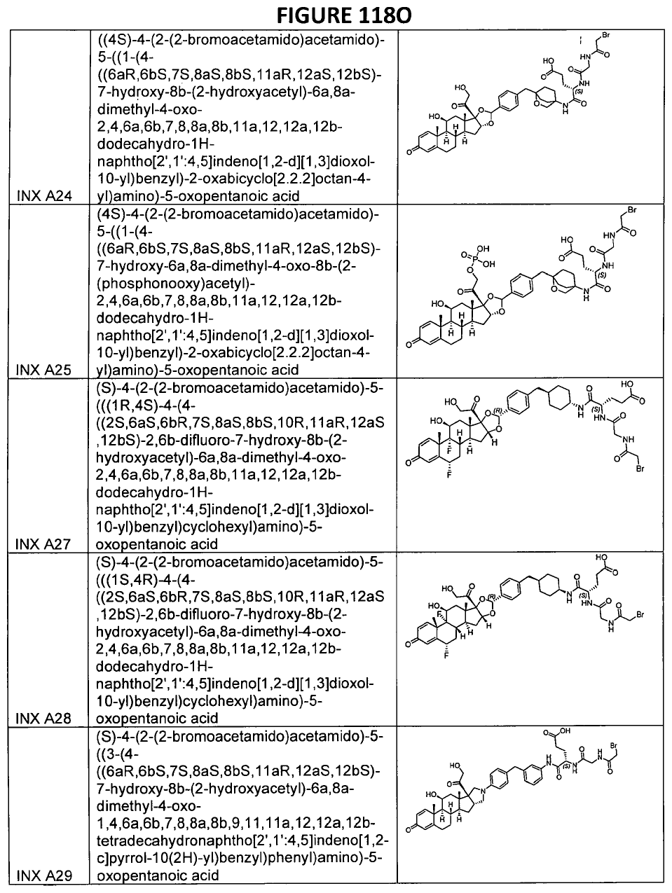

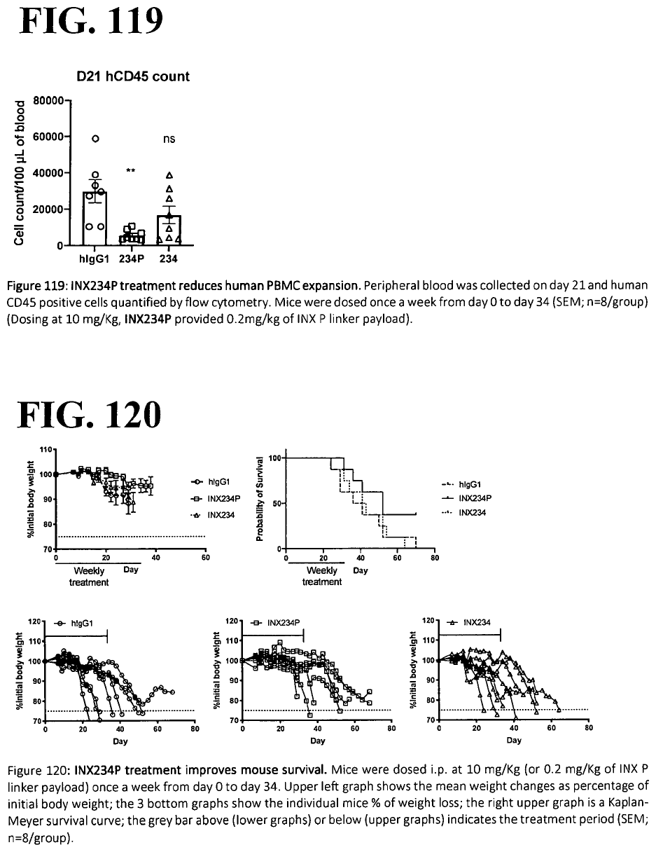

To monitor xeno-GvHD progression, we measured human T cell expansion and weight loss. As shown in FIG. 119, human PBMC numbers were significantly reduced in the INX234P treated group on day 21 post cell transfer. In these experiments peripheral blood was collected on day 21 and human CD45 positive cells quantified by flow cytometry. Mice were dosed once a week from day 0 to day 34 (SEM; n=8/group) (Dosing at 10 mg/Kg, INX234P provided 0.2 mg/kg of INX P linker payload).

### INX234P Treatment Improves Mouse Survival

INX234P treatment was also shown to prevent weight loss as compared to the hlgG1 and naked Ab control groups which translated to improved survival. Once treatment was terminated, INX234P treated mice started losing weight. INX234P treatment led to improved survival as shown by a median survival of 52 days vs 38.5 days and 42 days for the human IgG1 and INX234 treated groups respectively.

As shown by the experimental results in FIG. 120, INX234P treatment improves mouse survival. In these experiments the mice were dosed i.p. at 10 mg/Kg (or 0.2 mg/Kg of INX P linker payload) once a week from day 0 to day 34. The upper left graph shows the mean weight changes as percentage of initial body weight; the 3 bottom graphs show the individual mice % of weight loss; the right upper graph is a Kaplan-Meyer survival curve; the grey bar above (lower graphs) or below (upper graphs) indicates the treatment period (SEM; n=8/group).

### Experiment 2

### INX234V/P Treatment Prevents Human PBMC Expansion

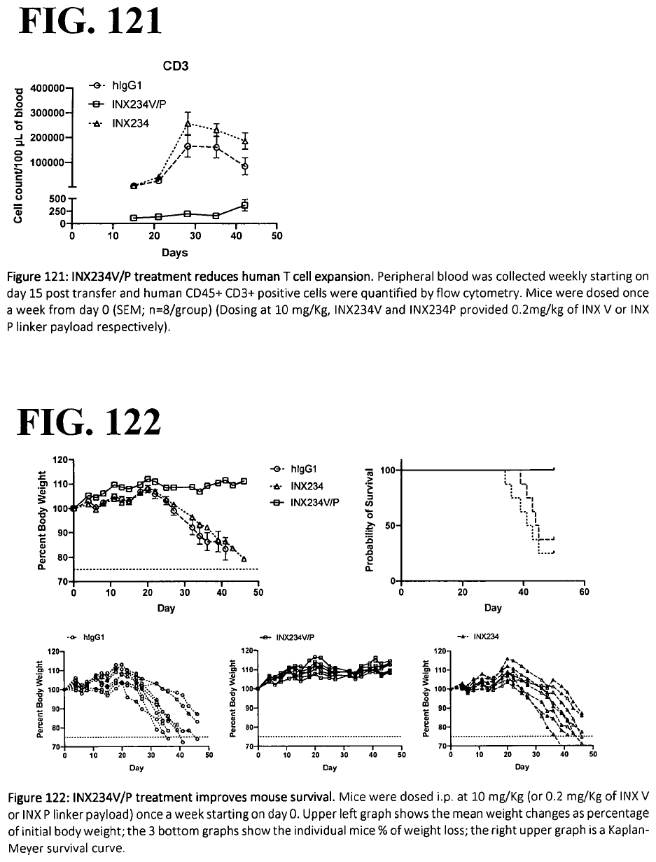

In this experiment, animals were treated with INX234V once a week until day 27 and then with INX234P for the remainder of the experiment and afterward we followed human T cell expansion over time, as mainly (>98%) T cells expand in this model. As shown in FIG. 121, as early as day 15, a dramatic difference in cell number could be observed. While both control groups display cell expansion that plateaued at day 28, the INX234V/P treated group showed consistently low T cell numbers.

Particularly in the experiments in FIG. 121, peripheral blood was collected weekly starting on day 15 post transfer and human CD45⁺ CD3⁺ positive cells were quantified by flow cytometry. Mice were dosed once a week from day 0 (SEM; n=8/group) (Dosing at 10 mg/Kg, INX234V and INX234P provided 0.2 mg/kg of INX V or INX P linker payload respectively).

### INX234V/P Treatment Improves Mouse Survival

INX234V/P treatment further completely prevented weight loss as compared to the hlgG1 and unconjugated Ab control groups which translated to dramatically improved survival (FIG. 122) with a median survival for the human IgG1 and unconjugated INX234 of 42 and 44.5 days respectively. All animals in the INX234V/P treated group were alive at the time of this report.

As shown by the results in FIG. 122, INX234V/P treatment improves mouse survival. In these experiments, the mice were dosed i.p. at 10 mg/Kg (or 0.2 mg/Kg of INX V or INX P linker payload) once a week starting on day 0. The upper left graph shows the mean weight changes as percentage of initial body weight; the 3 bottom graphs show the individual mice % of weight loss; the right upper graph is a Kaplan-Meyer survival curve.

### Conclusions

The data show that both INX234P and INX234V can control xeno-GvHD progression and increase survival by limiting/preventing human T cell expansion and disease development in immunodeficient mice. The data further show that changing from linker payload INX V to INX P in EXPERIMENT 2 maintained the efficacy of the treatment. This efficacy is mediated by the GC payload as demonstrated by the fact that the naked antibody INX234 elicited no efficacy.

### REFERENCES

1—Johnston R J, Su L J, Pinckney J, Critton D, Boyer E, Krishnakumar A, Corbett M, Rankin A L, Dibella R, Campbell L, Martin G H, Lemar H, Cayton T, Huang R Y, Deng X, Nayeem A, Chen H, Ergel B, Rizzo J M, Yamniuk A P, Dutta S, Ngo J, Shorts A O, Ramakrishnan R, Kozhich A, Holloway J, Fang H, Wang Y K, Yang Z, Thiam K, Rakestraw G, Rajpal A, Sheppard P, Quigley M, Bahjat K S, Korman A J. “VISTA is an acidic pH-selective ligand for PSGL-1.” Nature. 2019 October; 574 (7779): 565-570.
2—McPherson M J, et al. (2017) Glucocorticoid receptor agonist and immunoconjugates thereof, U.S. Ser. No. 15/611,037

### Example 25: Anti-VISTA Antibody Drug Conjugates have Efficacy in Mouse T Cell Transfer Colitis Model

Inflammatory bowel diseases (IBD) (Crohn's disease; ulcerative colitis) are chronic inflammatory disorders of the intestine and/or colon. While glucocorticoids (GC) are highly effective in treating IBD, these powerful anti-inflammatoire usually restricted to treating acute flare-ups due to toxicities associated with long term treatments.

The naïve T cell transfer mouse model of chronic colitis has been instrumental in delineating the immunological mechanisms responsible for the induction as well as regulation of intestinal inflammation. It is also the only murine colitis model that is highly responsive to GC.

The efficacy of the 2 antibody drug conjugates (ADC) INX234P and INX234V, both anti-human VISTA monoclonal antibodies linked to 2 different glucocorticoid (GC) payloads, was evaluated in the T cell transfer murine model of colitis.

Briefly, naïve CD4 T cells from human VISTA expressing mice (hVISTA KI) were transferred into immunodeficient Rag1−/− mice that do not produce mature T, B and NK cells. After >21 days, animals slowly start to lose weight and decreases in circulating naïve T cells are observed. In this model, the ADC targets only the transferred cells while the free GC can act on both transferred and host cells.

In EXPERIMENT 1, mice were treated with INX234P at 10 mg/Kg starting on day 0 and then once a week. In EXPERIMENT 2, treatment with INX234V was initiated on day 21 once the first signs of disease were noted and subsequently treated once a week. Four control groups were added to both experiments: PBS, dexamethasone (Dex) at 2 and 0.2 mg/Kg, and unconjugated INX234 antibody, all dosed once a week. In EXPERIMENT 1 treatment was initiated on day 0 (prophylactic) and terminated on day 61, whereas in EXPERIMENT 2 treatment was initiated on day 21 (or therapeutic) and remains ongoing at the time of this filing.

The data obtained in both experiments show that both INX234P and INX234V have superior impact compared to either dose of Dex in
1) maintaining a population of engrafted naïve CD4 T cells
2) protecting against disease as demonstrated by dramatically increased survival

Moreover, the data also demonstrate that targeting exclusively the disease effector cells is sufficient to prevent disease development.

### Materials and Methods

### Study Design

In both experiments, all treatments were administered once a week.

For EXPERIMENT 1, INX234P treatment was initiated on day 0 (or prophylactic) after naïve CD4 T cell transfer and terminated on day 61. For EXPERIMENT 2, INX234V treatment was initiated on day 21 (or therapeutic) after naïve CD4 T cell transfer. In this experiment, on day 42, 49 and 56, animals received INX234P, and were subsequently switched back to INX234V for the rest of the treatment period.

### Test Agents and Dosage

### Antibodies

INX234 (ATUM, lot #72931.2.a) is a humanized anti-human VISTA antibody on a human IgG1/kappa backbone with L234A/L235A/E269R/K322A silencing mutations in the Fc region.
INX234P (Abzena, lot #JZ-0556-017-2) is INX234 with a drug/antibody ratio (DAR) of 8.0, conjugated via full modification of the interchain disulfides. The linker/payload (INX P) consists of a negatively charged protease sensitive linker with a budesonide analog payload (INX-SM-3).
INX234V (Abzena, Lot #RJS-1054-003) is the INX234 antibody with a DAR of 7.9, conjugated via modification of the interchain disulfides. The linker/payload (INX V) consists of a negatively charged protease sensitive linker with a budesonide analog payload (INX-SM-32).

All antibodies were diluted in PBS and injected intraperitoneal (i.p.) in a volume of 0.2 ml to deliver a dose of 10 mg/Kg.

### Dexamethasone

Dexamethasone sterile injection from Phoenix, NDC 57319-519-05, was diluted to a final volume of 0.2 ml in PBS and dosed as described via i.p. injection.

### Mice

8-week-old female Rag1−/− mice or B6.129S7-Rag1^{tm1Mom}/J were ordered from Jackson Laboratories (Stock No: 002216).
Human VISTA knock-in (hVISTA KI) mice have the human VISTA cDNA knocked-in in place of the mouse VISTA gene, and express human VISTA both at RNA and protein levels with the same expression pattern as mouse VISTA or C57BI/6 mice. The hVISTA KI mice were bred on site (Center for Comparative Medicine and Research at Dartmouth). Female mice 8-10-week-old were used for cell transfer.

### Disease Model Set Up

### Naïve CD4 T Cell Isolation

Naïve CD4 T cells were isolated from hVISTA KI spleen using an EasySep™ Mouse Naïve CD4+ T cell Isolation kit from StemCell (catalog #19765) following manufacturer instruction.

### Colitis Initiation

Following the protocol of Ostanin et al (2008), 0.5×106 naïve CD45RB+, CD4+ T cells were injected i.p. on day 0

| Antibody | Dilution |
|---|---|
| CD62L FITC | 200 |
| Mouse VISTA PE | 200 |
| CD45 PerCP cy5.5 | 400 |
| CD45RB | 200 |
| PECy7 | |
| Human VISTA | 200 |
| APC | |
| CD4 APC-cy7 | 200 |
| CD11b BV421 | 200 |
| Mouse Fc | 200 |
| block | |

### Whole Blood Immunostaining

Peripheral blood was harvested from the retro-orbital cavity using a glass Pasteur pipette that was first rinsed with heparin to prevent coagulation.

The 1—wash protocol described below allows for absolute blood cell count.

10 ml of antibody cocktail (see table on the right) was directly added to 100 ml of blood. After 30 min incubation at room temperature (RT), 600 ml BD FACS lysis buffer was added to the sample. After 30 min incubation at RT, samples were spun at 550 rcf for 5 min, washed once in PBS, and then resuspended in a fixed volume of PBS. The whole sample was run on a MacsQuant flow cytometer to obtain an absolute cell number.

### Disease Monitoring

Mice were weighed 3 times a week and euthanized once weight drops below 75% of their initial weight as allowed by our IACUC organization.

### Results

### Experiment 1

### INX234P Treatment Improves Survival

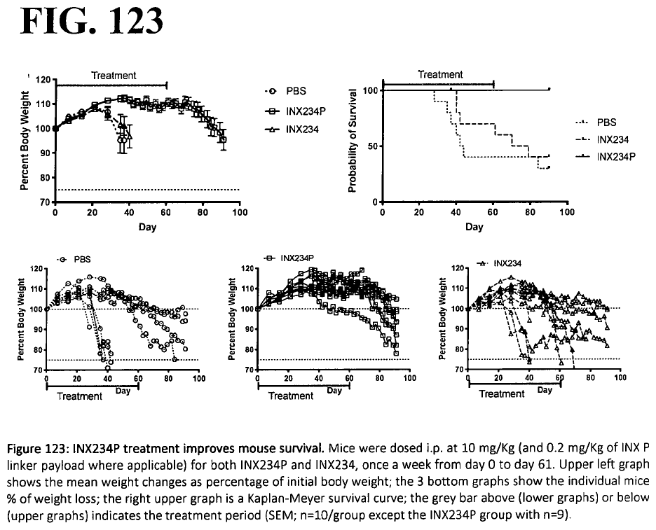

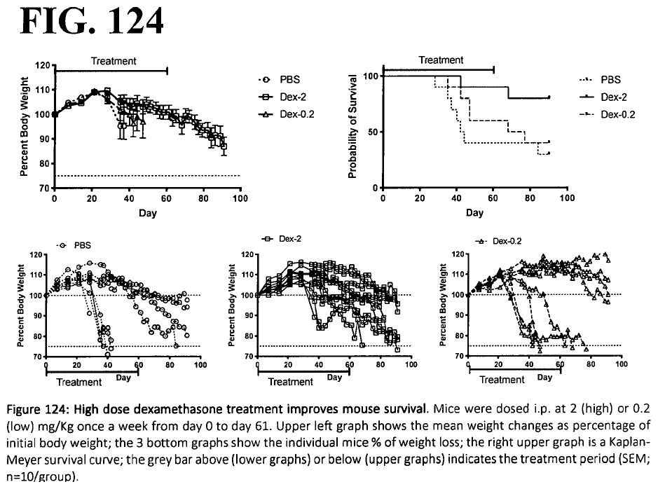

As shown in FIG. 123 and FIG. 124, the control groups, PBS and unconjugated (EXPERIMENT 1) and the low Dex (0.2 mg/Kg) group showed median survival of 43, 74.5 and 72.5 days respectively. In contrast, by experiment termination on day 91, neither the INX234P nor the high Dex (2 mg/Kg) group had reached median survival. Moreover, while 20% (2 out of 10) of the mice had to be sacrificed in the high Dex group, all the mice survived in the INX234P treated group (except for 1 animal which was censored due to an early unrelated event). By 10 days following the end of the treatment on day 61, INX234P treated animals had started losing weight (FIG. 123 and FIG. 124).

More particularly, in the experiments in FIG. 123, mice were dosed i.p. at 10 mg/Kg (and 0.2 mg/Kg of INX P linker payload where applicable) with both INX234P and INX234, once a week from day 0 to day 61. The upper left graph shows the mean weight changes as percentage of initial body weight; the 3 bottom graphs show the individual mice % of weight loss; the right upper graph is a Kaplan-Meyer survival curve; the grey bar above (lower graphs) or below (upper graphs) indicates the treatment period (SEM; n=10/group except the INX234P group with n=9).

The data in FIG. 124 show that high dose dexamethasone treatment improved mouse survival. In the experiments the mice were dosed i.p. at 2 (high) or 0.2 (low) mg/Kg once a week from day 0 to day 61. Upper left graph shows the mean weight changes as percentage of initial body weight; the 3 bottom graphs show the individual mice % of weight loss; the right upper graph is a Kaplan-Meyer survival curve; the grey bar above (lower graphs) or below (upper graphs) indicates the treatment period (SEM; n=10/group).

### Experiment 2

In this experiment, we evaluated the ability of anti-VISTA conjugated to the linker payload INX V to provide therapeutic efficacy, by initiating treatment on day 21 post naïve T cell transfer. On day 42, 49 and 56, animals received INX234P, and were subsequently switched back to INX234V for the rest of the treatment period.

### INX234V Treatment Improves Survival

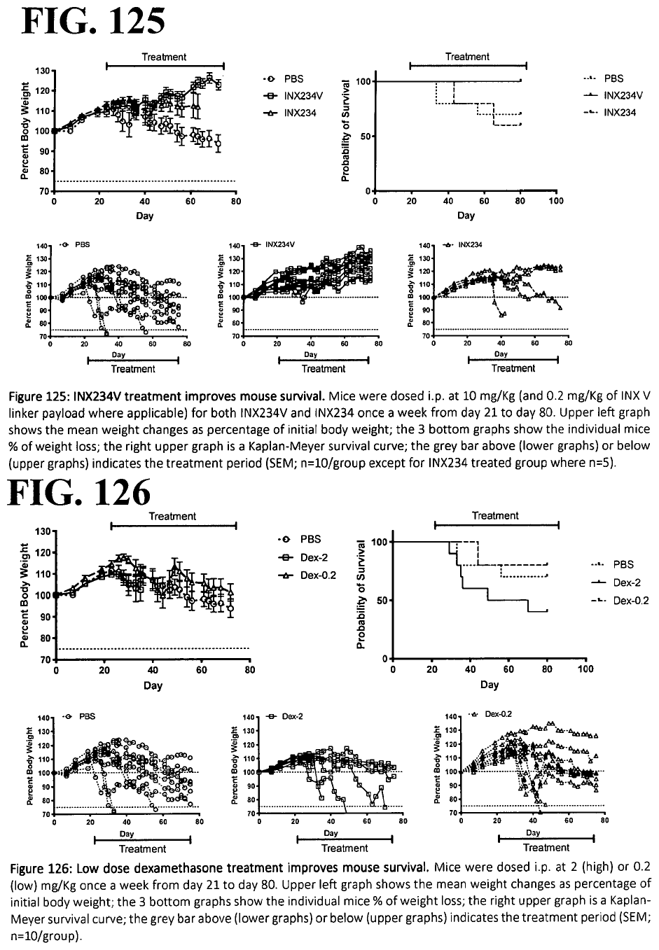

As shown by the data in FIGS. 125 and 126, the weight of the animals treated with INX234V steadily increased in the course of the experiment (which is normal) while all the other groups showed, after some weight gain in the first 30 days, decreases over time.

While at the time of the report, median survival was not reached for most groups, the fact that there is no weight loss in the ADC treated group will likely translate to dramatically improved survival. Moreover, surprisingly, the group treated with Dex at high dose, showed decreased survival when compared to all the other groups including the PBS group (FIG. 126)

The data in FIG. 125 show that INX234V treatment improves mouse survival. In these experiments the mice were dosed i.p. at 10 mg/Kg (and 0.2 mg/Kg of INX V linker payload where applicable) for both INX234V and INX234 once a week from day 21 to day 80. The upper left graph shows the mean weight changes as percentage of initial body weight; the 3 bottom graphs show the individual mice % of weight loss; the right upper graph is a Kaplan-Meyer survival curve; the grey bar above (lower graphs) or below (upper graphs) indicates the treatment period (SEM; n=10/group except for INX234 treated group where n=5).

The data in FIG. 126 show that low dose dexamethasone treatment improves mouse survival. In these experiments the mice were dosed i.p. at 2 (high) or 0.2 (low) mg/Kg once a week from day 21 to day 80. Upper left graph shows the mean weight changes as percentage of initial body weight; the 3 bottom graphs show the individual mice % of weight loss; the right upper graph is a Kaplan-Meyer survival curve; the grey bar above (lower graphs) or below (upper graphs) indicates the treatment period (SEM; n=10/group).

INX234V Treatment does not Affect the Survival of the Transferred T Cells but Limits their Activation.

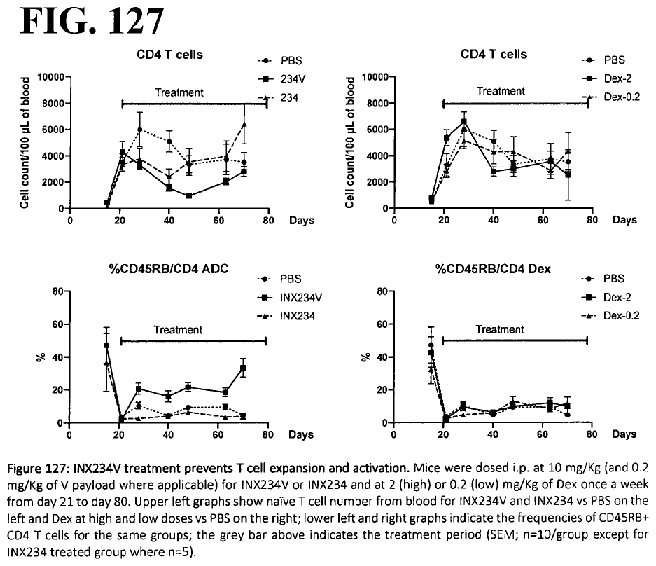

Colitis development is caused by the expansion and activation of the transferred naïve CD4 T cells. To monitor disease development, we measured T cell numbers and activation status in peripheral blood, on a weekly basis. T cell activation was measured as a loss of CD45RB expression, a naïve cell marker. Treatment was initiated on day 21, once we observed the sharp drop in the frequencies of CD45^{RB+} CD4 T cells as compared to day 7 where ˜50% of T cells were still CD45^{RB+} (FIG. 127, lower graphs). Animals were randomized at enrollment for treatment.

As shown in FIG. 127, while there are no differences in T cell numbers between PBS control group and Dex treated groups (upper right graph), INX234V treated mice showed very consistent limited expansion of T cells (upper left graph). The group treated with the unconjugated antibody displayed lower T cell numbers only during the first 2 weeks of treatment (upper left graph). Additionally, the frequency of CD45^{RB+} T cells or naïve T cells dropped dramatically between day 15 and 21 in all groups, before treatment initiation; INX234V treatment allowed the maintenance of the naïve T cell population as >20% of the total T cell engrafted (bottom left graph), while all the other groups displayed a frequency of naïve cells of <10% (bottom left and right graph).

The data in FIG. 127 show that INX234V treatment prevents T cell expansion and activation. In these experiments the mice were dosed i.p. at 10 mg/Kg (and 0.2 mg/Kg of V payload where applicable) with INX234V or INX234 and at 2 (high) or 0.2 (low) mg/Kg of Dex once a week from day 21 to day 80. Upper left graphs show naïve T cell number from blood for INX234V and INX234 vs PBS on the left and Dex at high and low doses vs PBS on the right; lower left and right graphs indicate the frequencies of CD45RB+CD4 T cells for the same groups; the grey bar above indicates the treatment period (SEM; n=10/group except for INX234 treated group where n=5).

### Conclusions

The data show that both INX234P and INX234V have superior efficacy compared to Dex both prophylactically and therapeutically in
1) maintaining a population of engrafted naïve CD4 T cells
2) protecting against disease as demonstrated by dramatically increased survival

Moreover, the data also demonstrate that targeting exclusively the disease effector cells is sufficient to prevent disease development.

### REFERENCES

McPherson M J, et al. (2017) Glucocorticoid receptor agonist and immunoconjugates thereof, U.S. Ser. No. 15/611,037
Dmitry V. Ostanin et al. (2009) T cell transfer model of chronic colitis: concepts, considerations, and tricks of the trade, Am J Physiol Gastrointest Liver Physiol.; 296 (2): G135-G146.

### Summary of Exemplary Advantages of Subject ADCs

The experimental results disclosed in this application show that the subject ADCs possess a unique combination of advantages compared to previous ADCs for targeting and directing internalization of anti-inflammatory agents, particularly steroids into immune cells, e.g., ADCs which target CD74, CD163, TNF, and PRLR; because of the combined benefits of VISTA as an ADC target and the specific properties of the anti-VISTA antibody which is comprised in the subject ADCs (binds to VISTA expressing immune cells at physiologic pH and possesses a very short pK).

These advantages include the following:
1) The subject ADCs bind to immune cells which express VISTA at very high density and notwithstanding their very short PK are efficacious (elicit anti-inflammatory activity) for prolonged duration (possess long PD's), and therefore are well suited for treating chronic or episodic inflammatory or autoimmune or allergic diseases wherein prolonged and repeated administration is therapeutically warranted.
2) The subject ADCs target a broad range of immune cells including neutrophils, myeloid, T cells and endothelium, therefore the subject ADCs may be used to treat diseases inflammatory or autoimmune or allergic diseases involving any or all of these types of immune cells.
3) The subject ADCs have a rapid onset of efficacy (as short as within 2 hours) and therefore may be used for acute treatment.
4) The subject ADCs do not bind B cells and therefore should not be as immunosuppressive as free steroids (i.e., humoral immunity will be retained). This potentially will reduce toxicity or adverse side effects during chronic or prolonged usage of the subject ADCs which has been associated with the prolonged usage of free steroids (e.g., prolonged steroid use has been correlated to some cancers, infectious conditions, and other diseases, apparently because of the adverse effects of prolonged immunosuppression).
5) The subject ADCs act on Tregs which are an important immune cell responsible for steroid efficacy.
6) The subject ADCs act on both resting and activated immune cells such as myeloid cells, monocytes, eosinophils, dendritic cells, Tregs, CD8 T cells, CD4 T cells, NK cells, macrophages, neutrophils, (constitutively expressed thereon); consequently the subject ADCs will be active (elicit anti-inflammatory activity) both in active and remission phases of inflammatory and autoimmune conditions.
7) The subject ADCs act on neutrophils, which immune cells are critical for acute inflammation, further evidencing that the ADCs are well suited for treating acute inflammation, and for controlling bouts of inflammation, e.g., associated with the active phase of a chronic or episodic autoimmune or inflammatory condition, early in onset, ideally before pathologic symptoms manifest. This potentially will reduce tissue damage which can occur even before the subject experiences pain or other symptoms associated with inflammation.
8) The subject ADCs internalize immune cells very rapidly and constitutively because VISTA cell surface turnover is high.
9) The subject ADCs possess a very short half-life (PK) and only bind immune cells, therefore the subject ADCs should not be prone to target related toxicities and undesired peripheral steroid exposure (low non-specific loss effects).
10) The subject ADCs biological activity (anti-inflammatory action) in some embodiments is entirely attributable to the anti-inflammatory payload (steroid) because the anti-VISTA antibody possessing a silent IgG therein shows no immunological functions (no blocking of any VISTA biology) thereby potentially simplifying dosing and/or potentially avoiding adverse side effects, e.g., in individuals wherein VISTA agonism may not be therapeutically desirable.
11) The subject ADCs' biological activity (anti-inflammatory or immunosuppressive action) in some embodiments is attributable to both the anti-inflammatory payload (steroid) and to the Fc portion of the anti-VISTA antibody, particularly in embodiments wherein the anti-VISTA antibody comprises a functional IgG2 Fc region because the binding of anti-VISTA antibodies possessing a functional IgG2 to VISTA expressing immune cells agonizes the immunosuppressive effects of VISTA, particularly its suppressive effects on T cell proliferation and T cell activity, thereby providing an ADC with immunosuppressive activity elicited by 2 distinct mechanisms.

### Advantages of Anti-VISTA ADC

1) VISTA is highly expressed on selected immune cells
   Targets drug to neutrophils, myeloid, T cells and endothelium
   Does not bind B cells and thus may not be as immunosuppressive as free Glucocorticoid
   Expressed on Tregs (main cell type target for Glucocorticoid in lymphocytes)
   One of the only targets expressed on neutrophils (critical for acute inflammation)
   Internalizes rapidly and constitutively.
   VISTA cell surface turnover is high as well.
2) Anti-VISTA shows short half-life thus reducing any target related toxicities and reduces peripheral GC exposure (low non-specific loss effects)
3) Silent IgG shows no immunological functions (no blocking of any VISTA biology).
   Other binding antibody ADC candidates have independent functions on targets: CD74, CD163, TNF, PRLR

### Advantages of Glucocorticoid Linker Payloads

### INX P Series (INX P, INX S)

1) Enhancement of intracellular levels of released payload over literature comparator
2) Enhancement of potency of conjugated linker payload over conjugated comparator linker payload though comparator free payload has similar potency.
3) Allows stable drug antibody ratio of 8 with little to no aggregation at 150 mg/mL.
   Stable high concentration formulation allows potential for subcutaneous dosing

### INX V Series (INX V, INX A3, INX A11, INX A7, INX A23, INX A12)

1) Greatly enhanced intracellular levels of released payload over literature comparator
2) Conjugated linker payload has enhanced potency over other conjugated linker payloads with similar potency payloads
   Advantages of Glucocorticoid anti-VISTA ADC
1) Provides long intracellular exposure of Glucocorticoid due to high internalization into immune cells
2) Limited exposure of GC to non-immune cells due to <24-hour serum half-life of the ADC 3) Improved dosing due to higher potency of glucocorticoid pathway resulting from limited intracellular metabolism of drug
4) Stable drug antibody ratio of 8 with little to no aggregation at 150 mg/mL.
   Stable high concentration formulation allows potential for subcutaneous dosing

### REFERENCES CITED IN EXAMPLES

The following references and all other references cited in this application are incorporated by reference in their entireties.
(1) Johnston, R. J. et al. W. O. Publication. No. 2018/169993 A1.
(2) Graversen, J. H. et al. Mol Ther. 2012 August; 20 (8): 1550-1558
(3) Vafa, O. et al. Methods. 2014 January; 65 (1): 114-26.
(4) Durbin, K. R., Phipps, C., & Liao, X. (2018). Mechanistic Modeling of Antibody-Drug Conjugate Internalization at the Cellular Level Reveals Inefficient Processing Steps. Mol Cancer Ther, 1535-7163.
(5) Liao-Chan, S., Daine-Matsuoka, B., Heald, N., Wong, T., Lin, T., Cai, A. G., . . . . Theunissen, J. W. (2015). Quantitative assessment of antibody internalization with novel monoclonal antibodies against Alexa fluorophores. PLOS One, 10 (4): e012470.
(6) Liu, Z., Yu, Z., He, W. Liu, Z., Yu, Z., He, W., Ma, S., Sun, L., & Wang, L. (2009). “In-vitro internalization and in-vivo tumor uptake of anti-EGFR monoclonal antibody LA22 in A549 lung cancer cells and animal model”. Cancer Biother Radiopharm, 15-23.

| INFORMAL SEQUENCE LISTING | |
|---|---|
| Homo sapiens VISTA (Alternate names: B7-H5; B7H5; | |
| DD1alpha; GI24; PP2135; SISP1) AMINO ACID SEQUENCE | |
| SEQ ID NO: 1: | |
| 1 | mgvptaleag swrwgsllfa lflaaslgpv aafkvatpys lyvcpegqnv tltcrllgpv |
| 61 | dkghdvtfyk twyrssrgev qtcserrpir nltfqdlhlh hgghqaants hdlaqrhgle |
| 121 | sasdhhgnfs itmrnltlld sglycclvve irhhhsehrv hgamelqvqt gkdapsncvv |
| 181 | ypsssqdsen itaaalatga civgilclpl illlvykqrq aasnrraqel vrmdsniqgi |
| 241 | enpgfeaspp aqgipeakvr hplsyvaqrq psesgrhlls epstplsppg pgdvffpsld |
| 301 | pvpdspnfev i |
| Mus musculus VISTA AMINO ACID SEQUENCE | |
| SEQ ID NO: 2: | |
| 1 | mgvpavpeas sprwgtllla iflaasrglv aafkvttpys lyvcpegqna tltcrilgpv |
| 61 | skghdvtiyk twylssrgev qmckehrpir nftlqhlqhh gshlkanash dqpqkhglel |
| 121 | asdhhgnfsi tlrnvtprds glycclviel knhhpeqrfy gsmelqvqag kgsgstcmas |
| 181 | neqdsdsita aalatgaciv gilclplill lvykqrqvas hrraqelvrm dsntqgienp |
| 241 | gfettppfqg mpeaktrppl syvaqrqpse sgryllsdps tplsppgpgd vffpsldpvp |
| 301 | dspnseai |
| Mus musculus VISTA AMINO ACID SEQUENCE | |
| SEQ ID NO: 3: | |
| 1 | mgvpavpeas sprwgtllla iflaasrglv aafkvttpys lyvcpegqna tltcrilgpv |
| 61 | skghdvtiyk twylssrgev qmckehrpir nftlqhlqhh gshlkanash dqpqkhglel |
| 121 | asdhhgnfsi tlrnvtprds glycclviel knhhpeqrfy gsmelqvqag kgsgstcmas |
| 181 | neqdsdsita aalatgaciv gilclplill lvykqrqvas hrraqelvrm dssntqgien |
| 241 | pgfettppfq gmpeaktrpp Isyvaqrqps esgryllsdp stplsppgpg dvffpsldpv |
| 301 | pdspnseai |
| Homo sapiens VISTA (Alternate names: B7-H5; B7H5; | |
| DD1alpha; GI24; PP2135; SISP1) NUCLEIC ACID SEQUENCE | |
| SEQ ID NO: 4: | |
| 1 | gggggcgggt gcctggagca cggcgctggg gccgcccgca gcgctcactc gctcgcactc |
| 61 | agtcgcggga ggcttccccg cgccggccgc gtcccgcccg ctccccggca ccagaagttc |
| 121 | ctctgcgcgt ccgacggcga catgggcgtc cccacggccc tggaggccgg cagctggcgc |
| 181 | tggggatccc tgctcttcgc tctcttcctg gctgcgtccc taggtccggt ggcagccttc |
| 241 | aaggtcgcca cgccgtattc cctgtatgtc tgtcccgagg ggcagaacgt caccctcacc |
| 301 | tgcaggctct tgggccctgt ggacaaaggg cacgatgtga ccttctacaa gacgtggtac |
| 361 | cgcagctcga ggggcgaggt gcagacctgc tcagagcgcc ggcccatccg caacctcacg |
| 421 | ttccaggacc ttcacctgca ccatggaggc caccaggctg ccaacaccag ccacgacctg |
| 481 | gctcagcgcc acgggctgga gtcggcctcc gaccaccatg gcaacttctc catcaccatg |
| 541 | cgcaacctga ccctgctgga tagcggcctc tactgctgcc tggtggtgga gatcaggcac |
| 601 | caccactcgg agcacagggt ccatggtgcc atggagctgc aggtgcagac aggcaaagat |
| 661 | gcaccatcca actgtgtggt gtacccatcc tcctoccagg atagtgaaaa catcacggct |
| 721 | gcagccctgg ctacgggtgc ctgcatcgta ggaatcctct gcctccccct catcctgctc |
| 781 | ctggtctaca agcaaaggca ggcagcctcc aaccgccgtg cccaggagct ggtgcggatg |
| 841 | gacagcaaca ttcaagggat tgaaaacccc ggctttgaag cctcaccacc tgcccagggg |
| 901 | atacccgagg ccaaagtcag gcaccccctg tcctatgtgg cccagcggca gccttctgag |
| 961 | tctgggcggc atctgctttc ggagcccagc acccccctgt ctcctccagg ccccggagac |
| 1021 | gtcttcttcc catccctgga ccctgtccct gactctccaa actttgaggt catctagccc |
| 1081 | agctggggga cagtgggctg ttgtggctgg gtctggggca ggtgcatttg agccagggct |
| 1141 | ggctctgtga gtggcctcct tggcctcggc cctggttccc tccctcctgc tctgggctca |
| 1201 | gatactgtga catcccagaa gcccagcccc tcaacccctc tggatgctac atggggatgc |
| 1261 | tggacggctc agcccctgtt ccaaggattt tggggtgctg agattctccc ctagagacct |
| 1321 | gaaattcacc agctacagat gccaaatgac ttacatctta agaagtctca gaacgtccag |
| 1381 | cccttcagca gctctcgttc tgagacatga gccttgggat gtggcagcat cagtgggaca |
| 1441 | agatggacac tgggccaccc tcccaggcac cagacacagg gcacggtgga gagacttctc |
| 1501 | ccccgtggcc gccttggctc ccccgttttg cccgaggctg ctcttctgtc agacttcctc |
| 1561 | tttgtaccac agtggctctg gggccaggcc tgcctgccca ctggccateg ccaccttccc |
| 1621 | cagctgcctc ctaccagcag tttctctgaa gatctgtcaa caggttaagt caatctgggg |
| 1681 | cttccactgc ctgcattcca gtccccagag cttggtggtc ccgaaacggg aagtacatat |
| 1741 | tggggcatgg tggcctccgt gagcaaatgg tgtcttgggc aatctgaggc caggacagat |
| 1801 | gttgccccac ccactggaga tggtgctgag ggaggtgggt ggggccttct gggaaggtga |
| 1861 | gtggagaggg gcacctgccc cccgccctcc ccatccccta ctcccactgc tcagcgcggg |
| 1921 | ccattgcaag ggtgccacac aatgtcttgt ccaccctggg acacttctga gtatgaagcg |
| 1981 | ggatgctatt aaaaactaca tggggaaaca ggtgcaaacc ctggagatgg attgtaagag |
| 2041 | ccagtttaaa tctgcactct gctgctcctc ccccaccccc accttccact ccatacaatc |
| 2101 | tgggcctggt ggagtcttcg cttcagagcc attcggccag gtgcgggtga tgttcccatc |
| 2161 | tcctgcttgt gggcatgccc tggctttgtt tttatacaca taggcaaggt gagtcctctg |
| 2221 | tggaattgtg attgaaggat tttaaagcag gggaggagag tagggggcat ctctgtacac |
| 2281 | tctgggggta aaacagggaa ggcagtgcct gagcatgggg acaggtgagg tggggctggg |
| 2341 | cagaccccct gtagcgttta gcaggatggg ggccccaggt actgtggaga gcatagtcca |
| 2401 | gcctgggcat ttgtctccta gcagcctaca ctggctctgc tgagctgggc ctgggtgctg |
| 2461 | aaagccagga tttggggcta ggcgggaaga tgttcgccca attgcttggg gggttggggg |
| 2521 | gatggaaaag gggagcacct ctaggctgcc tggcagcagt gagccctggg cctgtggcta |
| 2581 | cagccaggga accccacctg gacacatggc cctgcttcta agccccccag ttaggcccaa |
| 2641 | aggaatggtc cactgagggc ctcctgctct gcctgggctg ggccaggggc tttgaggaga |
| 2701 | gggtaaacat aggcccggag atggggctga cacctcgagt ggccagaata tgcccaaacc |
| 2761 | ccggcttctc ccttgtccct aggcagaggg gggtcccttc ttttgttccc tctggtcacc |
| 2821 | acaatgcttg atgccagctg ccataggaag agggtgctgg ctggccatgg tggcacacac |
| 2881 | ctgtcctccc agcactttgc agggctgagg tggaaggacc gcttaagccc aggtgttcaa |
| 2941 | ggctgctgtg agctgtgttc gagccactac actccagcct ggggacggag caaaactttg |
| 3001 | cctcaaaaca aattttaaaa agaaagaaag aaggaaagag ggtatgtttt tcacaattca |
| 3061 | tgggggcctg catggcagga gtggggacag gacacctgct gttcctggag tcgaaggaca |
| 3121 | agcccacagc ccagattccg gttctcccaa ctcaggaaga gcatgccctg ccctctgggg |
| 3181 | aggctggcct ggccccagcc ctcagctgct gaccttgagg cagagacaac ttctaagaat |
| 3241 | ttggctgcca gaccccaggc ctggctgctg ctgtgtggag agggaggcgg cccgcagcag |
| 3301 | aacagccacc gcacttcctc ctcagcttcc tctggtgcgg ccctgccctc tcttctctgg |
| 3361 | acccttttac aactgaacgc atctgggctt cgtggtttcc tgttttcagc gaaatttact |
| 3421 | ctgagctccc agttccatct tcatccatgg ccacaggccc tgcctacaac gcactaggga |
| 3481 | cgtccctccc tgctgctgct ggggaggggc aggctgctgg agccgccctc tgagttgccc |
| 3541 | gggatggtag tgcctctgat gccagccctg gtggctgtgg gctggggtgc atgggagagc |
| 3601 | tgggtgcgag aacatggcgc ctccaggggg cgggaggagc actaggggct ggggcaggag |
| 3661 | gctcctggag cgctggattc gtggcacagt ctgaggccct gagagggaaa tccatgcttt |
| 3721 | taagaactaa ttcattgtta ggagatcaat caggaattag gggccatett acctatctcc |
| 3781 | tgacattcac agtttaatag agacttcctg cctttattcc ctcccaggga gaggctgaag |
| 3841 | gaatggaatt gaaagcacca tttggagggt tttgctgaca cagcggggac tgctcagcac |
| 3901 | tccctaaaaa cacaccatgg aggccactgg tgactgctgg tgggcaggct ggccctgcct |
| 3961 | gggggagtcc gtggcgatgg gcgctggggt ggaggtgcag gagccccagg acctgctttt |
| 4021 | caaaagactt ctgcctgacc agagctccca ctacatgcag tggcccaggg cagaggggct |
| 4081 | gatacatggc ctttttcagg gggtgctcct cgcggggtgg acttgggagt gtgcagtggg |
| 4141 | acagggggct gcaggggtcc tgccaccacc gagcaccaac ttggcccctg gggtcctgcc |
| 4201 | tcatgaatga ggccttcccc agggctggcc tgactgtgct gggggctggg ttaacgtttt |
| 4261 | ctcagggaac cacaatgcac gaaagaggaa ctggggttgc taaccaggat gctgggaaca |
| 4321 | aaggcctctt gaagcccagc cacagcccag ctgagcatga ggcccagccc atagacggca |
| 4381 | caggccacct ggcccattcc ctgggcattc cctgctttgc attgctgctt ctcttcaccc |
| 4441 | catggaggct atgtcaccct aactatcctg gaatgtgttg agagggattc tgaatgatca |
| 4501 | atatagcttg gtgagacagt gccgagatag atagccatgt ctgccttggg cacgggagag |
| 4561 | ggaagtggca gcatgcatgc tgtttcttgg ccttttctgt tagaatactt ggtgctttcc |
| 4621 | aacacacttt cacatgtgtt gtaacttgtt tgatccaccc ccttccctga aaatcctggg |
| 4681 | aggttttatt gctgccattt aacacagagg gcaatagagg ttctgaaagg tctgtgtctt |
| 4741 | gtcaaaacaa gtaaacggtg gaactacgac taaa |
| // | |
| Homo sapiens VISTA (Alternate names: B7-H5; B7H5; | |
| DD1alpha; GI24; PP2135; SISP1) CODING NUCLEIC | |
| ACID SEQUENCE | |
| SEQ ID NO: 5: | |
| 1 | ctcgccgcgc tgagccgcct cgggacggag ccatgcggcg ctgggcctgg gccgcggtcg |
| 61 | tggtccccct cgggccgcag ctcgtgctcc togggggcgt cggggcccgg cgggaggcac |
| 121 | agaggacgca gcagcctggc cagcgcgcag atccccccaa cgccaccgcc agcgcgtcct |
| 181 | cccgcgaggg gctgcccgag gcccccaagc catcccaggc ctcaggacct gagttctccg |
| 241 | acgcccacat gacatggctg aactttgtcc ggcggccgga cgacggcgcc ttaaggaagc |
| 301 | ggtgcggaag cagggacaag aagccgcggg atctcttcgg tcccccagga cctccaggtg |
| 361 | cagaagtgac cgcggagact ctgcttcacg agtttcagga gctgctgaaa gaggccacgg |
| 421 | agcgccggtt ctcagggctt ctggacccgc tgctgcccca gggggcgggc ctgcggctgg |
| 481 | tgggcgaggc ctttcactgc cggctgcagg gtccccgccg ggtggacaag cggacgctgg |
| 541 | tggagctgca tggtttccag gctcctgctg cccaaggtgc cttcctgcga ggctccggtc |
| 601 | tgagcctggc ctcgggtcgg ttcacggccc ccgtgtccgg catcttccag ttctctgcca |
| 661 | gtctgcacgt ggaccacagt gagctgcagg gcaaggcccg gctgcgggcc cgggacgtgg |
| 721 | tgtgtgttct catctgtatt gagtccctgt gccagcgcca cacgtgcctg gaggccgtct |
| 781 | caggcctgga gagcaacagc agggtcttca cgctacaggt gcaggggctg ctgcagctgc |
| 841 | aggctggaca gtacgcttct gtgtttgtgg acaatggctc cggggccgtc ctcaccatcc |
| 901 | aggcgggctc cagcttctcc gggctgctcc tgggcacgtg agggcgccca ggggggctgg |
| 961 | cgaggagctg ccgccggatc ccggggaccc tcctactgat gcccgtggtc accacaataa |
| 1021 | agagccctcc accctcaaaa aaaaaaaaaa aaaaa |
| // | |
| Mus musculus VISTA CODING NUCLEIC ACID SEQUENCE | |
| SEQ ID NO: 6: | |
| 1 | ctcgccgcgc tgagccgcct cgggacggag ccatgcggcg ctgggcctgg gccgcggtcg |
| 61 | tggtccccct cgggccgcag ctcgtgctcc tcgggggcgt cggggcccgg cgggaggcac |
| 121 | agaggacgca gcagcctggc cagcgcgcag atccccccaa cgccaccgcc agcgcgtcct |
| 181 | cccgcgaggg gctgcccgag gcccccaagc catcccaggc ctcaggacct gagttctccg |
| 241 | acgcccacat gacatggctg aactttgtcc ggcggccgga cgacggcgcc ttaaggaagc |
| 301 | ggtgcggaag cagggacaag aagccgcggg atctcttcgg tcccccagga cctccaggtg |
| 361 | cagaagtgac cgcggagact ctgcttcacg agtttcagga gctgctgaaa gaggccacgg |
| 421 | agcgccggtt ctcagggctt ctggacccgc tgctgcccca gggggcgggc ctgcggctgg |
| 481 | tgggcgaggc ctttcactgc cggctgcagg gtccccgccg ggtggacaag cggacgctgg |
| 541 | tggagctgca tggtttccag gctcctgctg cccaaggtgc cttcctgcga ggctccggtc |
| 601 | tgagcctggc ctcgggtcgg ttcacggccc ccgtgtccgg catcttccag ttctctgcca |
| 661 | gtctgcacgt ggaccacagt gagctgcagg gcaaggcccg gctgcgggcc cgggacgtgg |
| 721 | tgtgtgttct catctgtatt gagtccctgt gccagcgcca cacgtgcctg gaggccgtct |
| 781 | caggcctgga gagcaacagc agggtcttca cgctacaggt gcaggggctg ctgcagctgc |
| 841 | aggctggaca gtacgcttct gtgtttgtgg acaatggctc cggggccgtc ctcaccatcc |
| 901 | aggcgggctc cagcttctcc gggctgctcc tgggcacgtg agggcgccca ggggggctgg |
| 961 | cgaggagctg ccgccggatc ccggggaccc tcctactgat gcccgtggtc accacaataa |
| 1021 | agagccctcc accctcaaaa aaaaaaaaaa aaaaa |

//
VSTB92 VH
SEQ ID NO: 10
QVQLVQSGAEVKKPGASVKVSCKASGYTFANYLIGWVRQAPGQRLEWMGDIYPGGGFISYNEKFKGRVTI
TRDTSASTAYMELSSLRSEDTAVYYCARRFDYGGYFFDYWGQGTLVTVSS
VSTB92_VL
SEQ ID NO: 11
DIVMTQSPLSLPVTPGEPASISCRSSQSIVHSNGNIYLEWYLQKPGQSPQLLIYKVSNRFSGVPDRFSGS
GSGTDFTLKISRVEAEDVGVYYCFQGSHVPWTFGQGTKLEIK
IgG1_LALA
SEQ ID NO: 12
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHRDPEVKFNWYVDGVEVHNAKTKPREEQYNSTYRVVSVLTVLHQDWLNGKEYKCAVSNKA
LPAPIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
IgG1_INXLALA
SEQ ID NO: 13
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRTP
EVTCVVVDVSHRDPEVKFNWYVDGVEVHNAKTKPREEQYNSTYRVVSVLTVLHQDWLNGKEYKCAVSNKA
LPAPIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
IgG2_sigma
SEQ ID NO: 14
ASTKGPSVFPLAPCSRSTSESTAALGCLVKDYFPEPVTVSWNSGALTSGVHTFPAVLQSSGLYSLSSVVT
VPSSNFGTQTYTCNVDHKPSNTKVDKTVERKCCVECPPCPAPPAAASSVFLFPPKPKDTLMISRTPEVTC
VVVDVSAEDPEVQFNWYVDGVEVHNAKTKPREEQFNSTFRVVSVLTVLHQDWLNGKEYKCKVSNKGLPSS
IEKTISKTKGQPREPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK
Human_Kappa_Constant
SEQ ID NO: 15
RTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC

1-132. (canceled)

133. A glucocorticoid agonist compound:
(A) having the following structure of Formula (I): 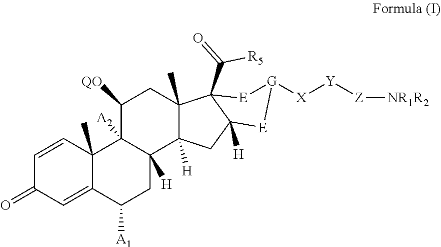
wherein
X is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo[2.2.2]octane, adamantane, and cubane each of which can be substituted with 1-4 heteroatoms independently selected from F, Cl, Br, I, N, S, and O, each of which ring structure may contain at least one skeletal heteroatom selected from N, S, and O, and are optionally further substituted with 1-4 C₁₋₃ alkyl or C1-3 perfluoroalkyl;
Z is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo2.2.2]octane, adamantane, and cubane each of which can be substituted with 1-4 heteroatoms independently selected from F, Cl, Br, I, N, S, and O, each of which ring structure may contain at least one skeletal heteroatom selected from N, S, and O, and are optionally further substituted with 1-4 C₁₋₃ alkyl or C1-3 perfluoroalkyl;
Y is selected from CHR₁, O, S, and NR₁;
E is selected from CH₂ and O;
G is selected from CH, and N;
further wherein when G is CH and X is phenyl, Z is not phenyl;
the linkage of G to X may optionally be selected from C₁₋₃ alkyl and ethylene oxide, each of which may be substituted with 1-4 heteroatoms independently selected from N, S, and O and are optionally further substituted with 1-4 C₁₋₃ alkyl;
the linkage of X to Z may occupy any available position on X and Z;
substituent NR₁R₂ may occupy any available position on Z;
R₁ is selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylaminoC(O)—;
when R₁ is H, R₂ may be selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylaminoC(O)—;
when R₁ is H, linear or branched alkyl of 1-8 carbons, or heteroaryl, R2 may be a functional group selected from
[(C═O)CH(W)NH]ₘ—[C═O]—[V]ₖ-J,
(C═O)OCH₂-p-aminophenyl-N—V-J,
(C═O)OCH₂-p-aminophenyl-N—[(C═O)CH(W)NH]ₘ—[C═O]—[V]ₖ-J, and
[V]ₖ—(C═O)OCH₂-p-aminophenyl-N—[(C═O)CH(W)NH]ₘ—[C═O]-J,
wherein m=1-6, k=0-1, and each permutation of W may independently be selected from H, [(CH₂)ₙR₃] where n=1-4, a branched alkyl chain terminating in R₃, and a linear or branched polyethylene oxide group comprising 1-13 units;
R₃ is selected from H, methyl, ethyl, isopropyl, OH, O-alkyl, NH₂, NH-alkyl, N-dialkyl, SH, S-alkyl, guanidine, urea, carboxylic acid, carboxamide, carboxylic ester, substituted or unsubstituted aryl, substituted or unsubstituted heteroaryl, wherein said aryl and heteroaryl substituents may be selected from alkyl, haloalkyl, halogen, biphenyl, nitro, nitrile, —OH, —O-alkyl, —NH2, alkylamino, dialkylamino, thiol, thioalkyl, guanidine, urea, carboxylic acid, alkoxyl, carboxamide, carboxylic ester, alkyl-C(O)O—, alkylamino-C(O)—, and dialkylamino-C(O)—;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH₂, N₃, thio, cyclooctyne, —OH, —CO₂H, trans-cyclooctene, alkynyl, propargyl, 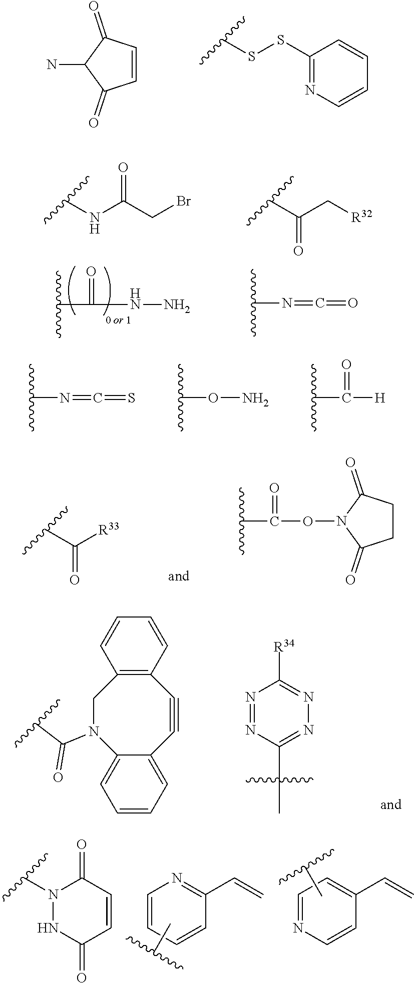
where R₃₂ is selected from Cl, Br, F, mesylate, and tosylate and R₃₃ is selected from Cl, Br, I, F, OH, —O—N-succinimidyl, —O-(4-nitrophenyl), —O-pentafluorophenyl or —O-tetrafluorophenyl R₃₄ is H, Me, tetrazine-H, and tetrazine-Me;
R₅ is selected from the group consisting of —CH₂OH, —CH₂SH, —CH₂Cl, —SCH₂Cl, —SCH₂F, —SCH₂CF₃, hydroxy, —OCH₂CN, —OCH₂Cl, —OCH₂F, —OCH₃, —OCH₂CH₃, —SCH₂CN, and 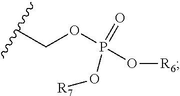
R₆ and R₇ are independently selected from hydrogen and C₁₋₁₀ alkyl;
Q may be H, 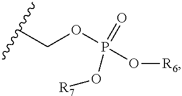
C(O)R₈ where R₈ is linear or branched alkyl of 1-8 carbons, or (C═O)NR₄CHₙNR₄(C═O)OCH₂—(V)ₙ-J where n=1-4 and R₄=H, alkyl or branched alkyl, or P(O)OR₄;
A₁ and A₂ are independently selected from H and F; and
unless otherwise specified, all possible stereoisomers are claimed; or
(B) of Formula (I): 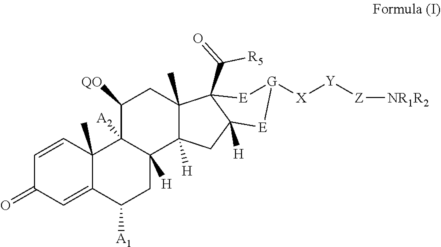
wherein
X is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo [2.2.2]octane, adamantane, and cubane each of which can be substituted with 1-4 heteroatoms independently selected from F, Cl, Br, I, N, S, and O, each of which ring structure may contain at least one skeletal heteroatom selected from N, S, and O, and are optionally further substituted with 1-4 C1-3 alkyl or C1-3 perfluoroalkyl;
Z is selected from phenyl, spiro[3.3]heptane, 3-6 membered heterocycle, cycloalkyl, spiro-alkyl, spiro-heterocycloalkyl, bicyclic alkyl, heterobicyclic alkyl, [1.1.1]bicyclopentane, bicyclo [2.2.2]octane, adamantane, and cubane each of which can be substituted with 1-4 heteroatoms independently selected from F, Cl, Br, I, N, S, and O, each of which ring structure may contain at least one skeletal heteroatom selected from N, S, and O, and are optionally further substituted with 1-4 C₁₋₃ alkyl or C1-3 perfluoroalkyl;
Y is selected from CHR₁, O, S, and NR₁;
E is selected from CH₂ and O;
G is selected from CH, and N;
further wherein when G is CH and X is phenyl, Z is not phenyl;
the linkage of G to X may optionally be selected from C₁₋₃ alkyl and ethylene oxide, each of which may be substituted with 1-4 heteroatoms independently selected from N, S, and O and are optionally further substituted with 1-4 C₁₋₃ alkyl;
the linkage of X to Z may occupy any available position on X and Z;
substituent NR₁R₂ may occupy any available position on Z;
R₁ is selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylamino-C(O)—;
when R₁ is H, R₂ may be selected from H, linear or branched alkyl of 1-8 carbons, aryl, and heteroaryl groups wherein said aryl and heteroaryl groups may be substituted with functional groups selected from alkyl, haloalkyl, halogen, biphenyl, nitro, nitrile, —OH, —O-alkyl, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)— and dialkylaminoC(O)—;
when R₁ is H, linear or branched alkyl of 1-8 carbons, or heteroaryl, R₂ may be a functional group selected from
[(C═O)CH(W)NH]ₘ—[C═O]—[V]ₖ-J,
(C═O)OCH₂-p-aminophenyl-N—V-J,
(C═O)OCH₂-p-aminophenyl-N—[(C═O)CH(W)NH]ₘ—[C═O]—[V]ₖ-J, and
[V]ₖ—(C═O)OCH₂-p-aminophenyl-N—[(C═O)CH(W)NH]ₘ—[C═O]-J,
wherein m=1-6, k=0-1, and each permutation of W may independently be selected from H, [(CH₂)ₙR₃] where n=1-4, a branched alkyl chain terminating in R₃, and a linear or branched polyethylene oxide group comprising 1-13 units;
R₃ is selected from H, methyl, ethyl, isopropyl, OH, O-alkyl, NH₂, NH-alkyl, N-dialkyl, SH, S-alkyl, guanidine, urea, carboxylic acid, carboxamide, carboxylic ester, substituted or unsubstituted aryl, substituted or unsubstituted heteroaryl, wherein said aryl and heteroaryl substituents may be selected from alkyl, haloalkyl, halogen, biphenyl, nitro, nitrile, —OH, —O-alkyl, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)—, and dialkylamino-C(O)—;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylamino-C(O)—;
J is a reactive group selected from —NH₂, N₃, thio, cyclooctyne, —OH, —CO₂H, trans-cyclooctene, alkynyl, propargyl, 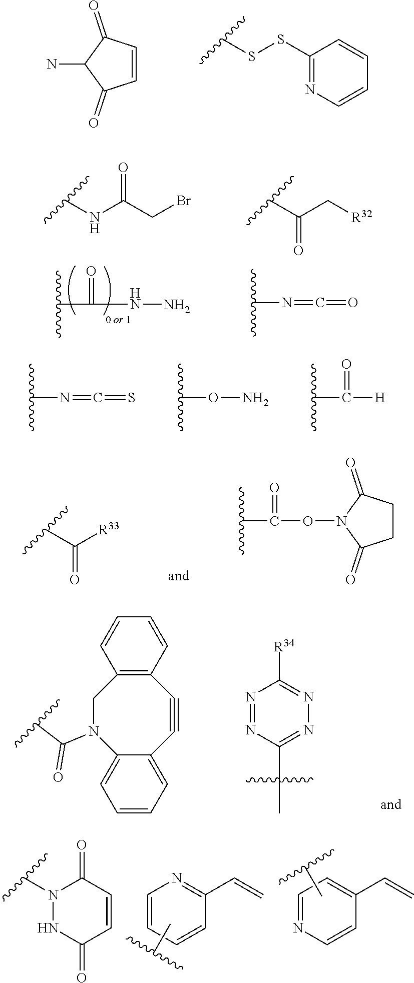
where R₃₂ is selected from Cl, Br, F, mesylate, and tosylate and R₃₃ is selected from Cl, Br, I, F, OH, —O—N-succinimidyl, —O-(4-nitrophenyl), —O-pentafluorophenyl or —O-tetrafluorophenyl R₃₄ is H, Me, tetrazine-H, and tetrazine-Me;
R₅ is selected from the group consisting of —CH₂OH, —CH₂SH, —CH₂Cl, —SCH₂Cl, —SCH₂F, —SCH₂CF₃, hydroxy, —OCH₂CN, —OCH₂Cl, —OCH₂F, —OCH₃, —OCH₂CH₃, —SCH₂CN, and 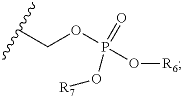
R₆ and R₇ are independently selected from hydrogen and C₁₋₁₀ alkyl;
Q may be H, 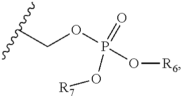
C(O)R₈ where R₈ is linear or branched alkyl of 1-8 carbons, or (C═O)NR₄CHₙNR₄(C═O)OCH₂—(V)ₙ-J where n=1-4 and R₄=H, alkyl or branched alkyl, or P(O)OR₄;
A₁ and A₂ are independently selected from H and F; and
unless otherwise specified, all possible stereoisomers are claimed; or
(C) which possesses the structure of Formula (II): 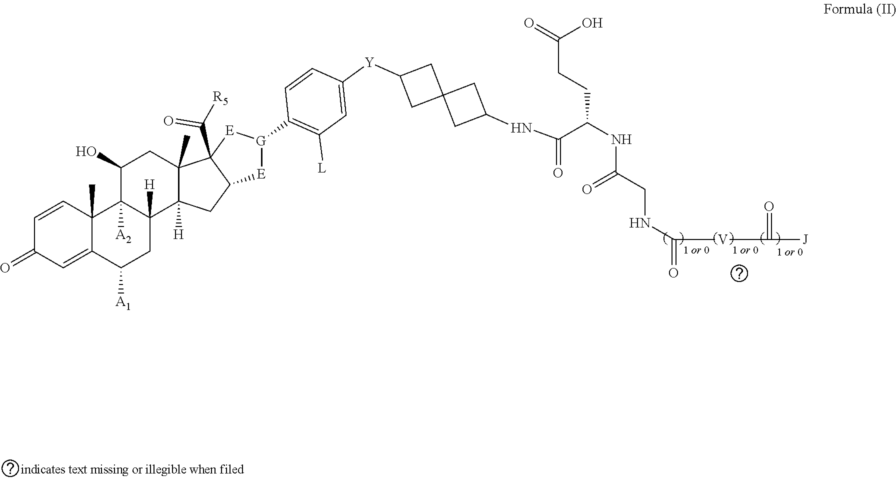
wherein
Y is selected from CH₂ and O;
E is selected from CH₂ and O;
G is selected from CH, and N;
L is selected from H and F;
R₅ is selected from —CH₂OH, —SCH₂F and 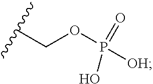 [SMILES: *COP(=O)(O)O]
A₁ and A₂ are independently selected from H and F;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH₂, N₃, thio, cyclooctyne, —OH, —CO₂H, trans-cyclooctene, alkynyl, propargyl, 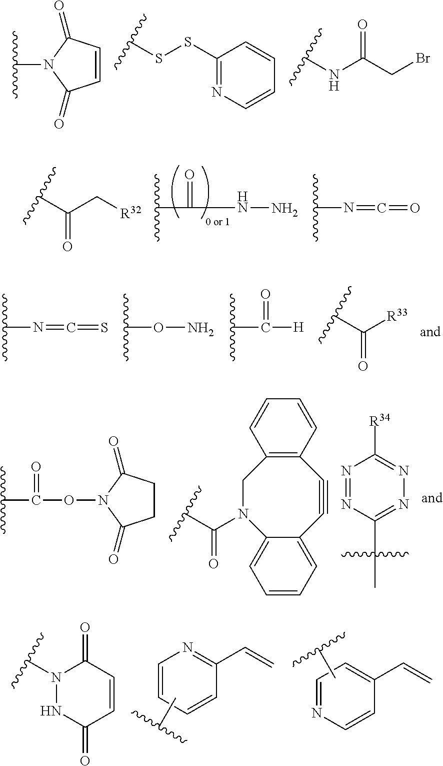
where R₃₂ is selected from Cl, Br, F, mesylate, and tosylate and R₃₃ is selected from Cl, Br, I, F, OH, —O—N-succinimidyl, —O-(4-nitrophenyl), —O-pentafluorophenyl or —O-tetrafluorophenyl R₃₄ is H, Me, tetrazine-H, and tetrazine-Me; or
(D) which possesses the structure of Formula (III): 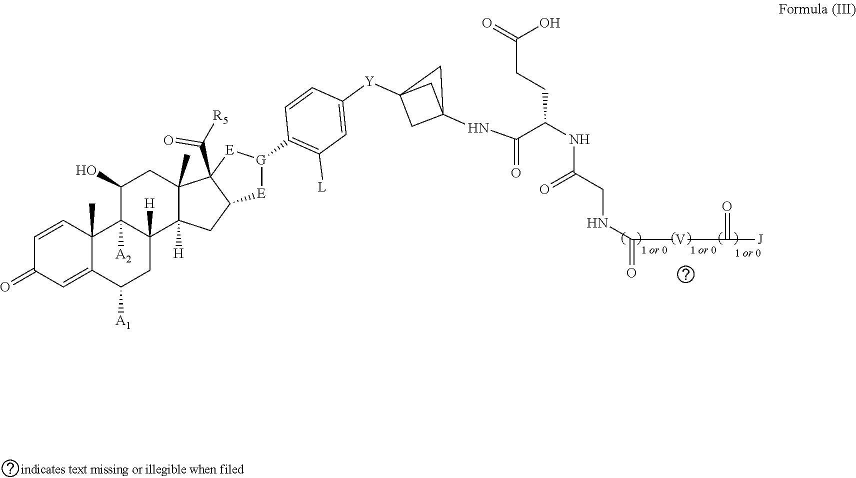
wherein
Y is selected from CH₂ and O;
E is selected from CH₂ and O;
G is selected from CH, and N;
L is selected from H and F;
R₅ is selected from —CH₂OH, —SCH₂F and 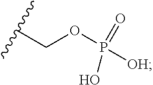 [SMILES: *COP(=O)(O)O]
A₁ and A₂ are independently selected from H and F;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—;
dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH₂, N₃, thio, cyclooctyne, —OH, —CO₂H, trans-cyclooctene, alkynyl, propargyl, 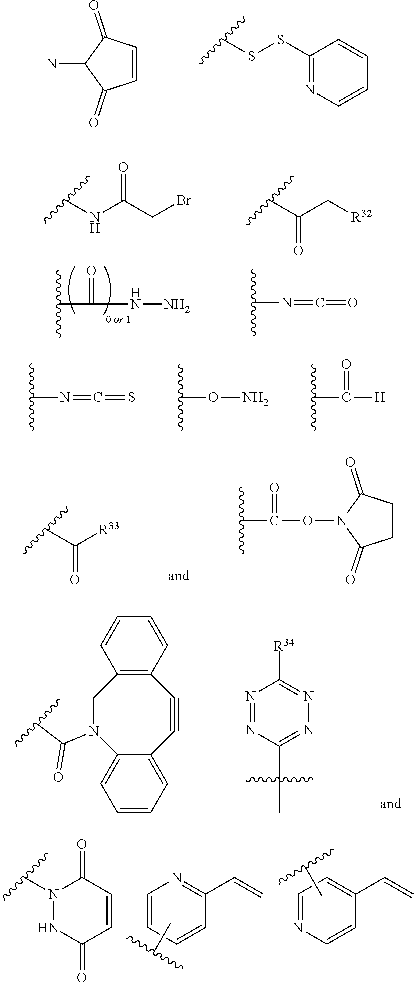
where R₃₂ is selected from Cl, Br, F, mesylate, and tosylate and R₃₃ is selected from Cl, Br, I, F, OH, —O—N-succinimidyl, —O-(4-nitrophenyl), —O-pentafluorophenyl or —O-tetrafluorophenyl R₃₄ is H, Me, tetrazine-H, and tetrazine-Me.

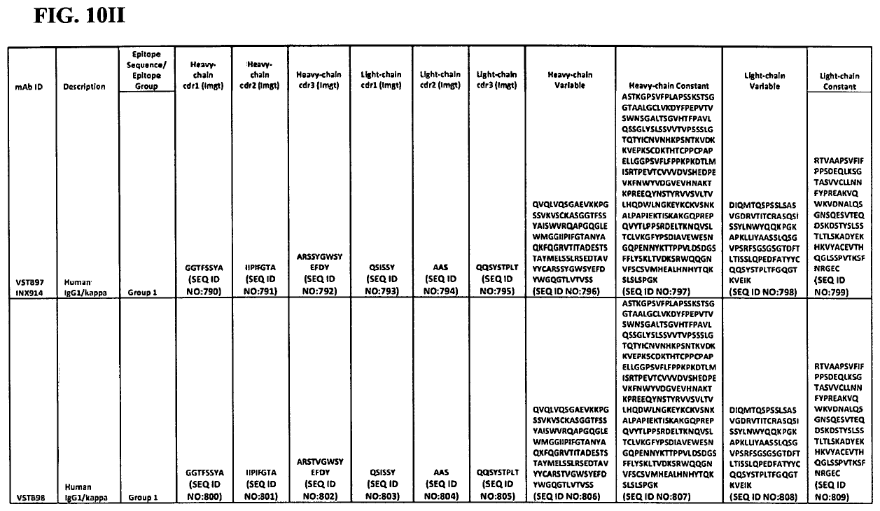

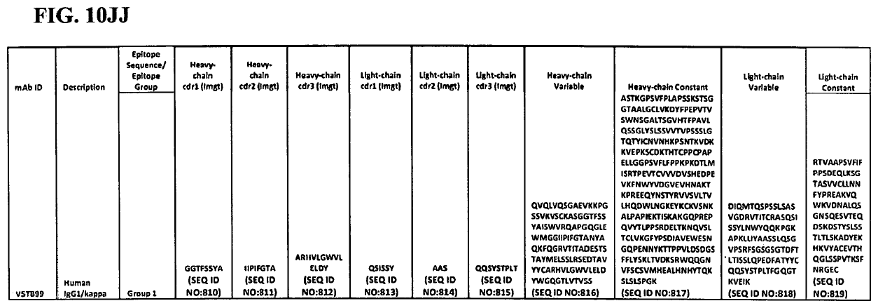

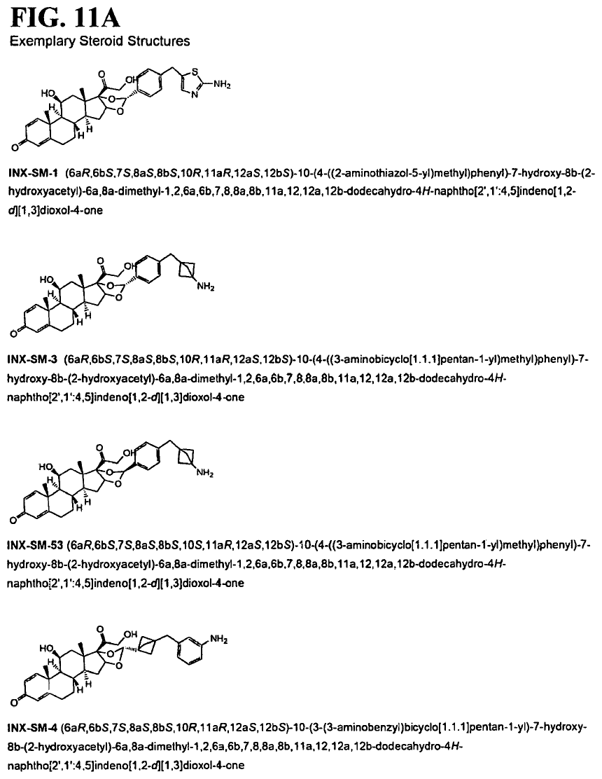

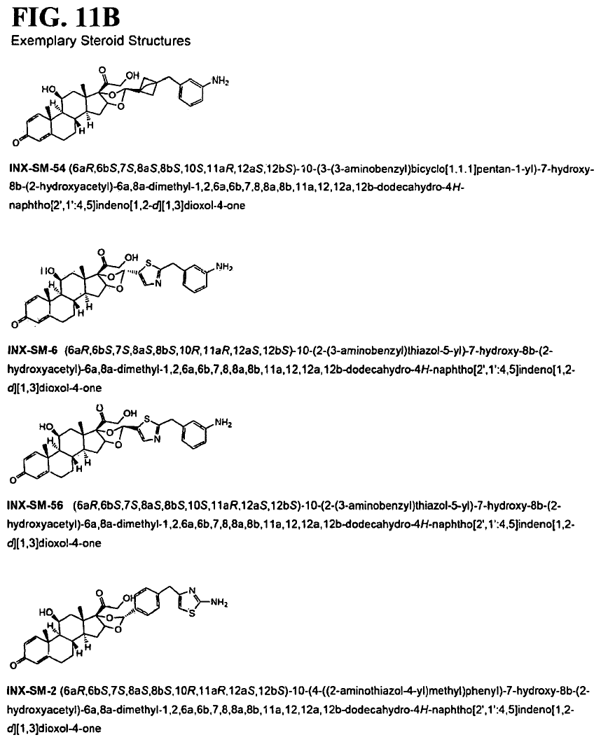

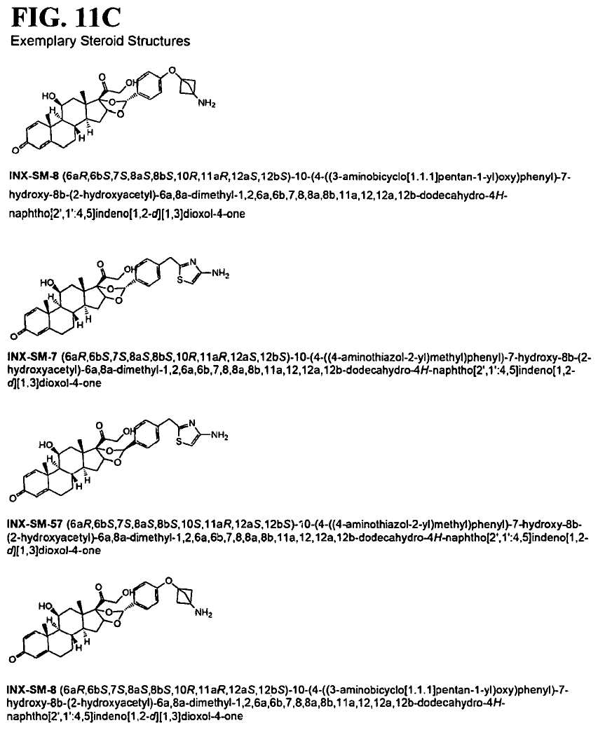

134. A glucocorticoid agonist compound, according to claim 133, selected from the following:
(a) a compound according to claim 133, embodiment (A), wherein X and Z are independently selected from phenyl, spiro[3.3]heptane, [1.1.1]bicyclopentane, and bicyclo[2.2.2]octane; Y is selected from CH₂ and O; permutations of W are independently selected from CH₂CH₂CO₂H and H, and further wherein when G is CH and X is phenyl, Z is not phenyl;
(b) a compound according to claim 133, embodiment (A), selected from any of the glucocorticoid agonist compounds disclosed in Example 3 or selected from those shown in FIG. 11 or FIG. 118A-O excluding INX J and INX L;
(c) a compound selected from the following: 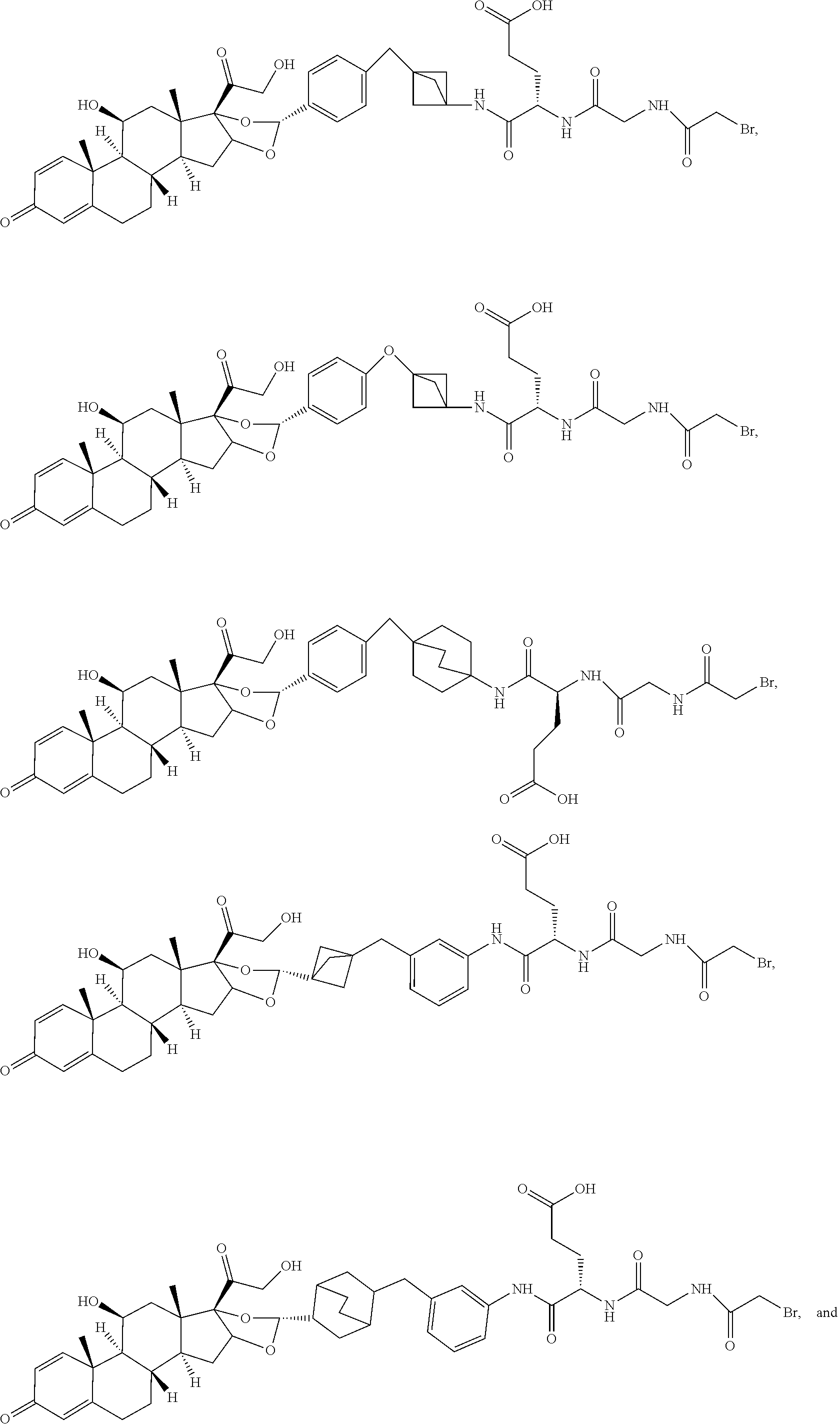 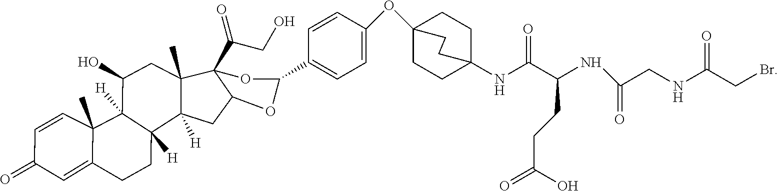
wherein Z is selected from 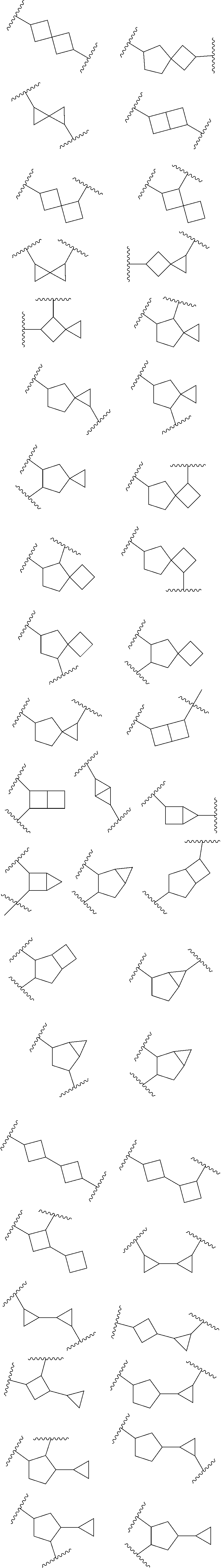 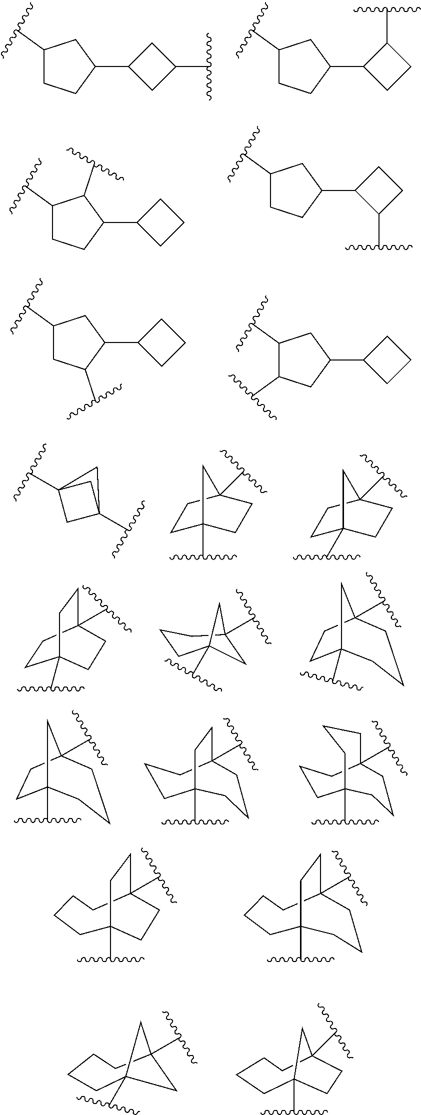
each of which may be substituted with 1-4 heteroatoms independently selected from F, Cl, Br, I, N, S, and O, and are optionally further substituted with 1-4 C₁₋₃ alkyl or C₁₋₃ perfluoroalkyl groups;
each of which ring structure may contain at least one additional skeletal heteroatom selected from N, S, and O; and
wherein each 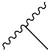
indicates a point of attachment to the rest of the formula and each of said points of attachment may be covalently bonded to the rest of the formula via an additional heteroatom selected from N, S, and O; or
(d) a compound according to claim 1, embodiment (B) or (C), wherein Z—NR₁ is selected from 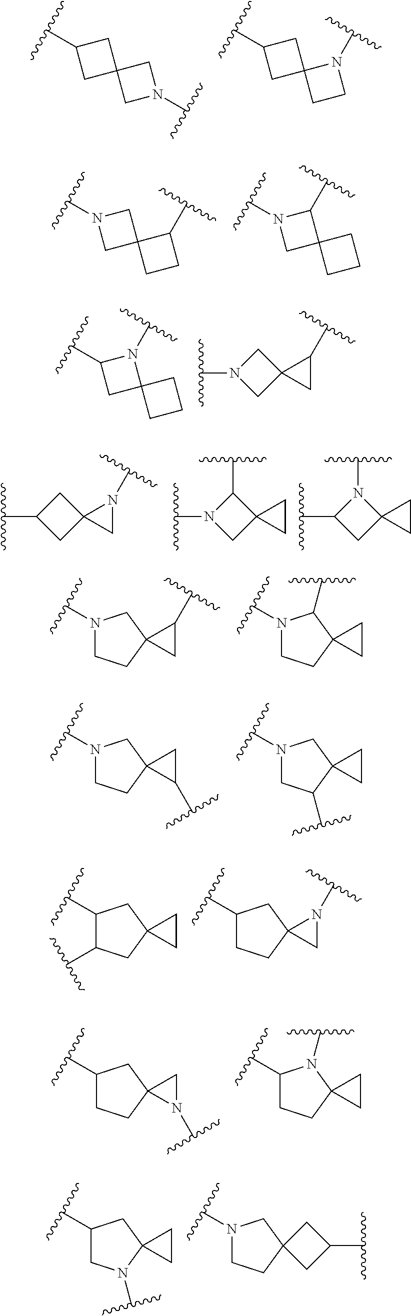 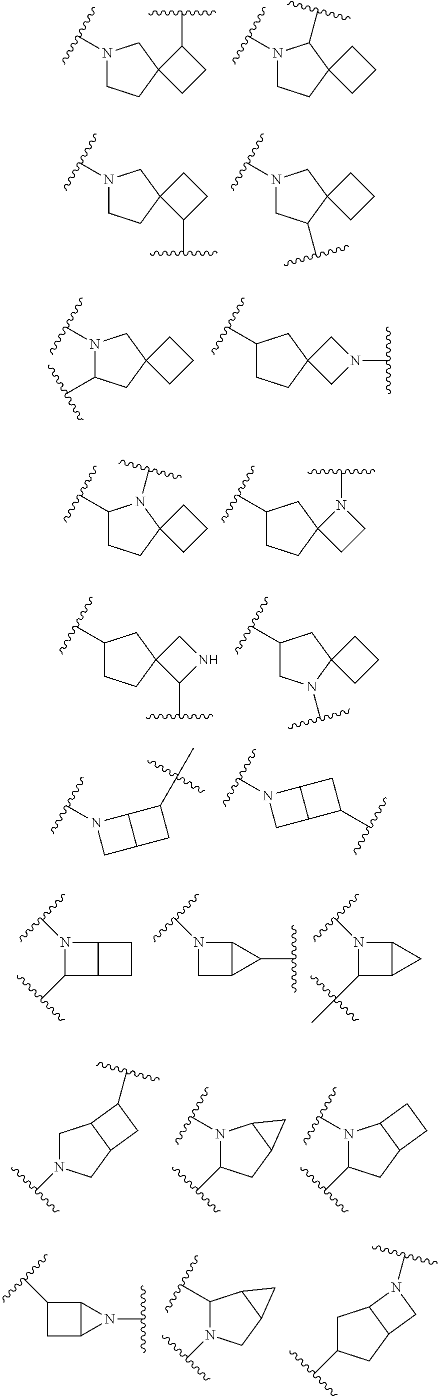 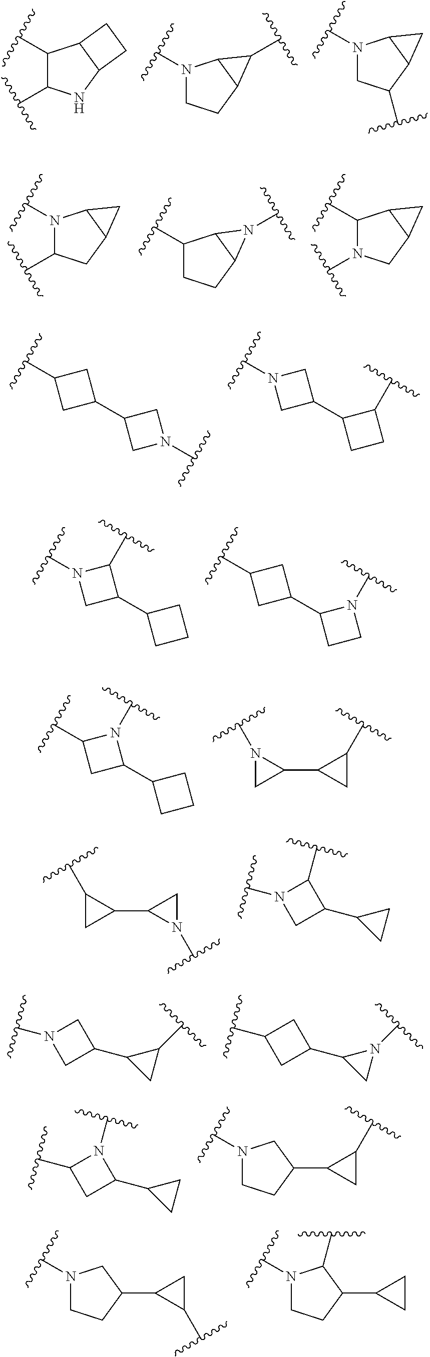 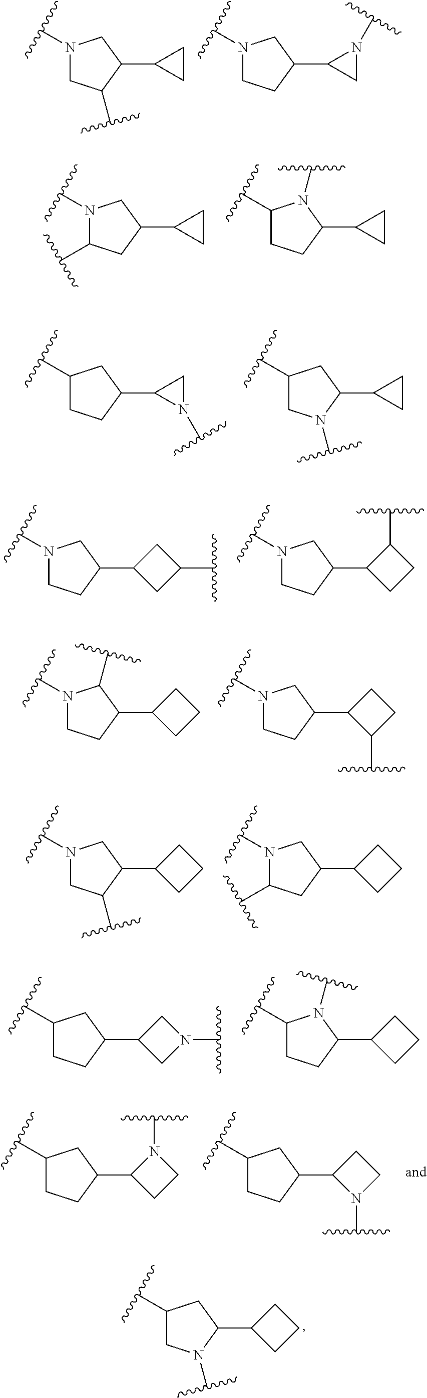
each of which may be substituted with 1-4 heteroatoms independently selected from F, Cl, Br, I, N, S, and O, and are optionally further substituted with 1-4 C₁₋₃ alkyl or C₁₋₃ perfluoroalkyl groups;
each of which ring structure may contain at least one additional skeletal heteroatom selected from N, S, and O; and
wherein each 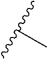
indicates a point of attachment to the rest of the formula and each of said points of attachment may be covalently bonded to the rest of the formula via an additional heteroatom selected from N, S, and O; or
(e) the glucocorticoid agonist compound possesses the structure of Formula (II): 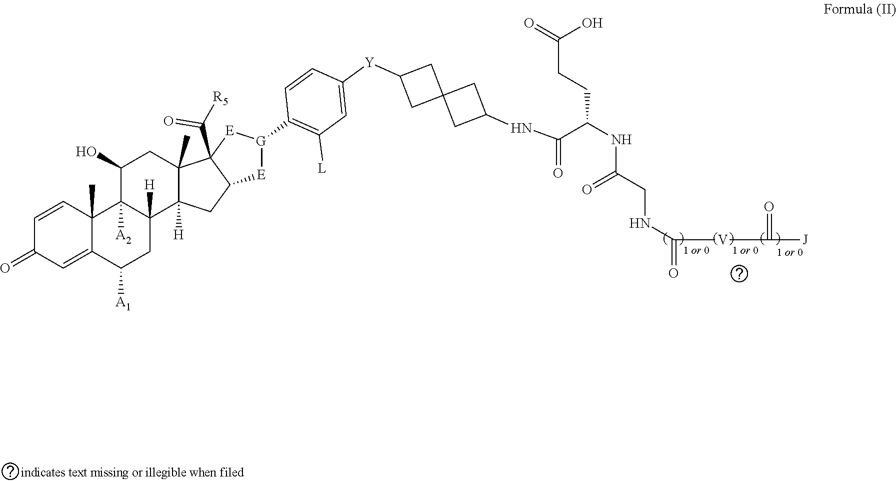
wherein
Y is selected from CH₂ and O;
E is selected from CH₂ and O;
G is selected from CH, and N;
L is selected from H and F;
R₅ is selected from —CH₂OH, —SCH₂F and 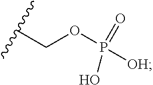 [SMILES: *COP(=O)(O)O]
A₁ and A₂ are independently selected from H and F;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH₂, N₃, thio, cyclooctyne, —OH, —CO₂H, trans-cyclooctene, alkynyl, propargyl, 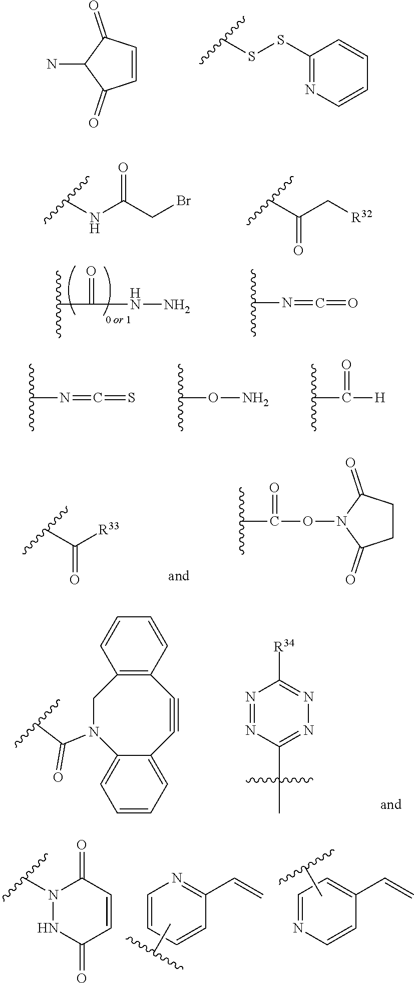
where R₃₂ is selected from Cl, Br, F, mesylate, and tosylate and R₃₃ is selected from Cl, Br, I, F, OH, —O—N-succinimidyl, —O-(4-nitrophenyl), —O-pentafluorophenyl or —O-tetrafluorophenyl R₃₄ is H, Me, tetrazine-H, and tetrazine-Me; or
(f) the glucocorticoid agonist compound possesses the structure of Formula (III): 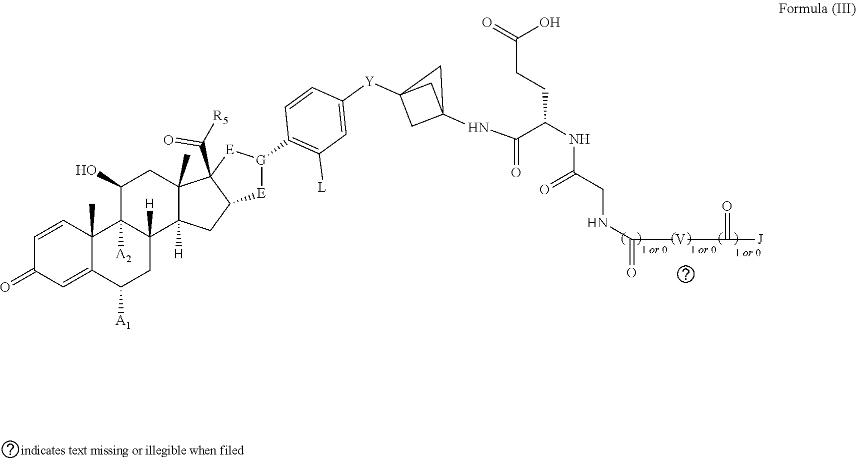
wherein
Y is selected from CH₂ and O;
E is selected from CH₂ and O;
G is selected from CH, and N;
L is selected from H and F;
R₅ is selected from —CH₂OH, —SCH₂F and 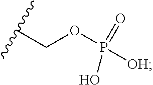 [SMILES: *COP(=O)(O)O]
A₁ and A₂ are independently selected from H and F;
V may be selected from an alkyl chain of 1-8 carbons; a linear or branched polyethylene oxide group comprising 1-13 units; linear or branched alkyl group comprising 1-8 carbons; —O-alkyl; carboxylic acid; carboxamide; carboxylic ester; alkyl-C(O)O—; alkylamino-C(O)—; dialkylaminoC(O)—; a 1-3 amino acid sequence wherein each amino acid is independently selected from Glu, Gly, Asn, Asp, Gln, Leu, Lys, Ala, betaAla, Phe, Val, and Cit; aryl; and heteroaryl groups wherein said aryl and heteroaryl groups may be substituted with functional groups selected from alkyl, haloalkyl, halogen, biphenyl, nitro, nitrile, —OH, —NH₂, alkylamino, dialkylamino, thiol, thioalkyl, guanidine, urea, carboxylic acid, alkoxyl, carboxamide, carboxylic ester, alkyl-C(O)O—, alkylamino-C(O)—, dialkylaminoC(O)—;
J is a reactive group selected from —NH₂, N₃, thio, cyclooctyne, —OH, —CO₂H, trans-cyclooctene, alkynyl, propargyl, 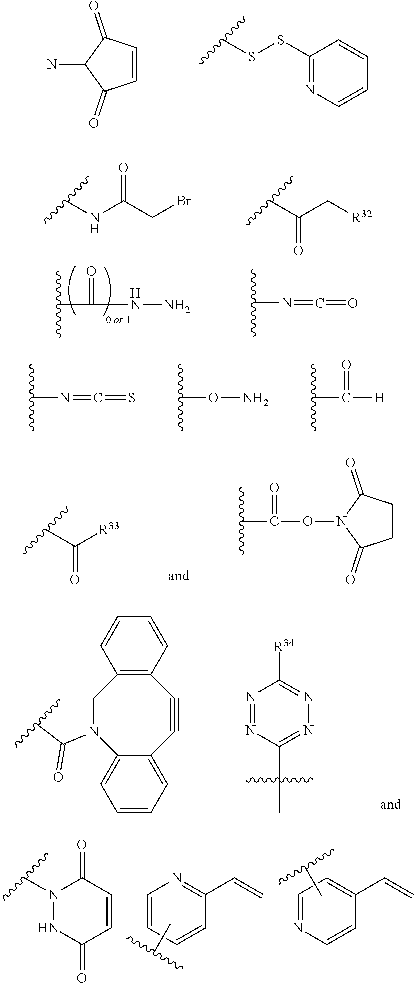
where R₃₂ is selected from Cl, Br, F, mesylate, and tosylate and R₃₃ is selected from Cl, Br, I, F, OH, —O—N-succinimidyl, —O-(4-nitrophenyl), —O-pentafluorophenyl or —O-tetrafluorophenyl R₃₄ is H, Me, tetrazine-H, and tetrazine-Me;
(g) the glucocorticoid agonist compound is selected from: 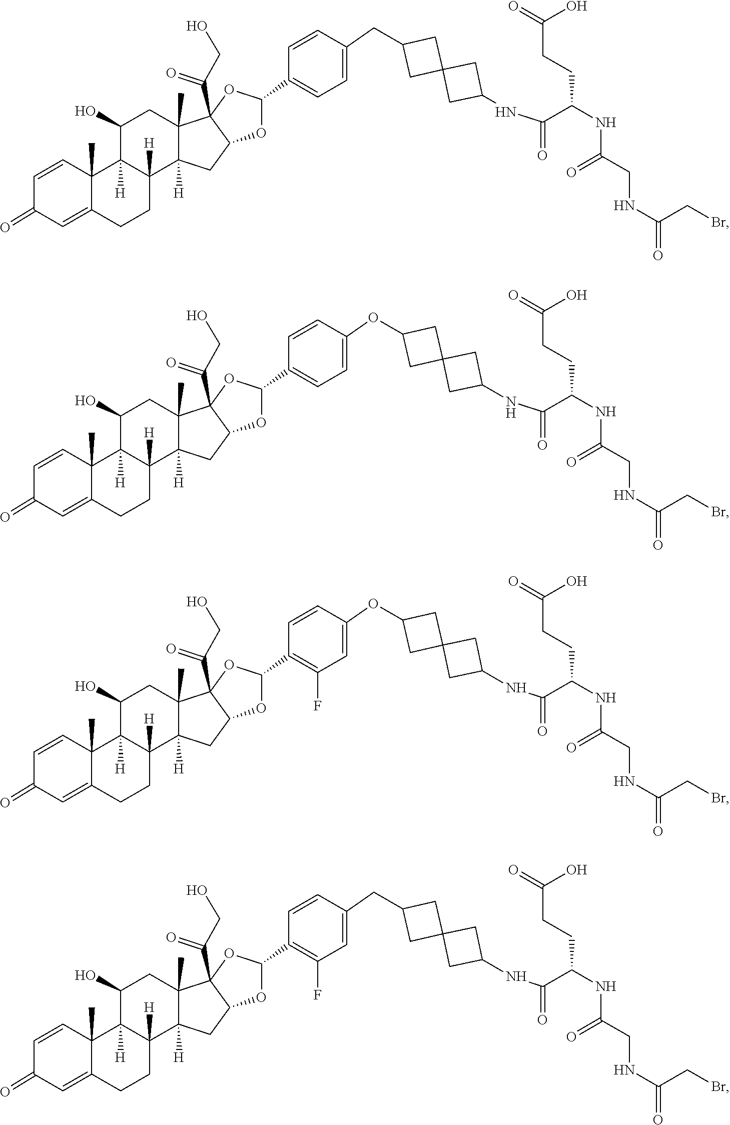 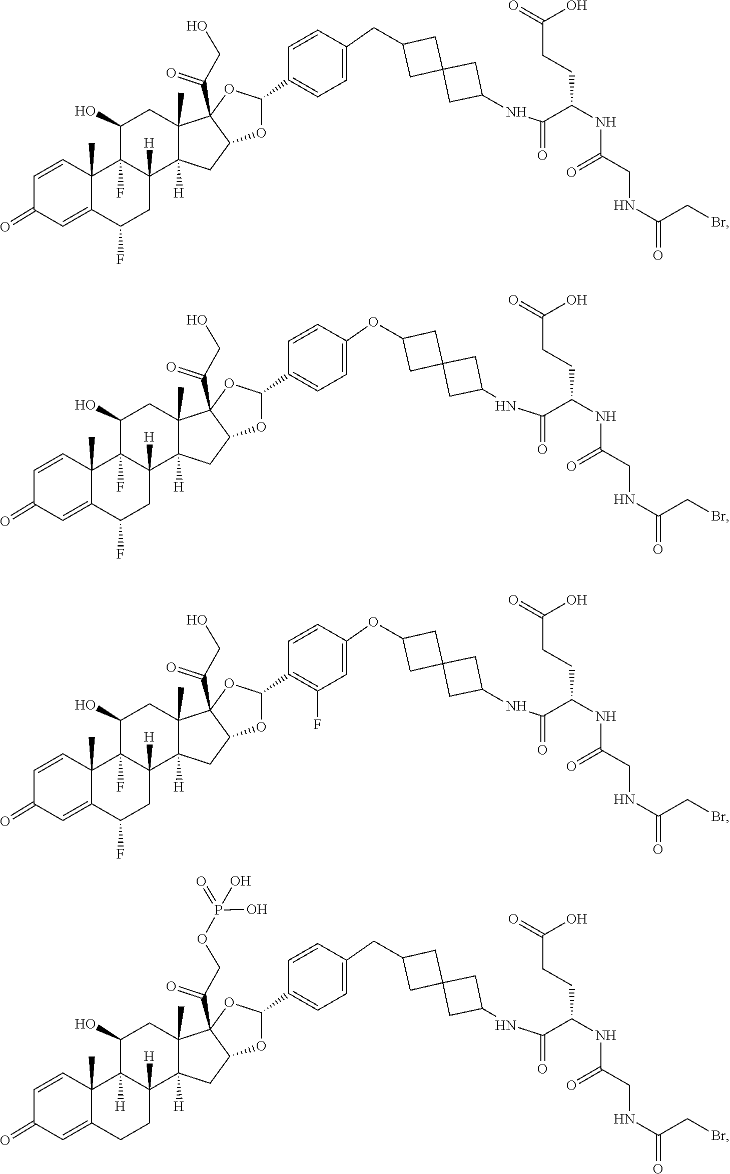 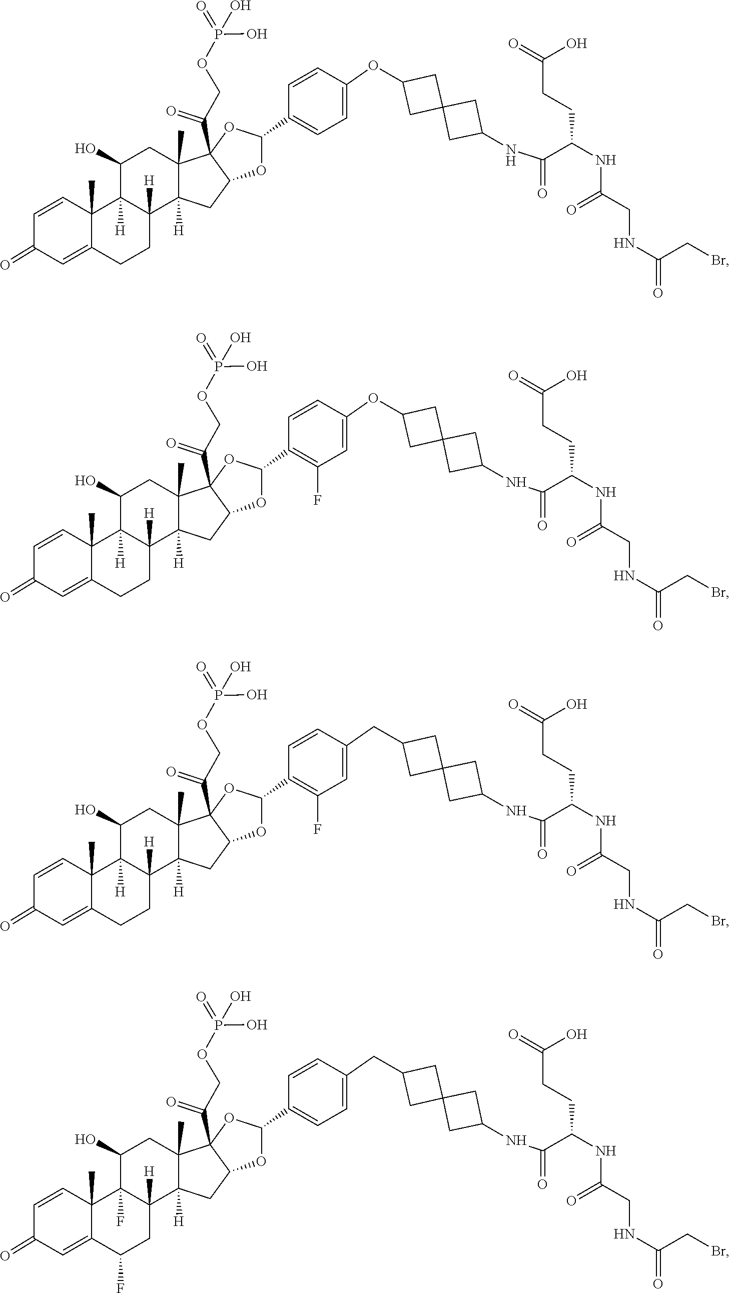 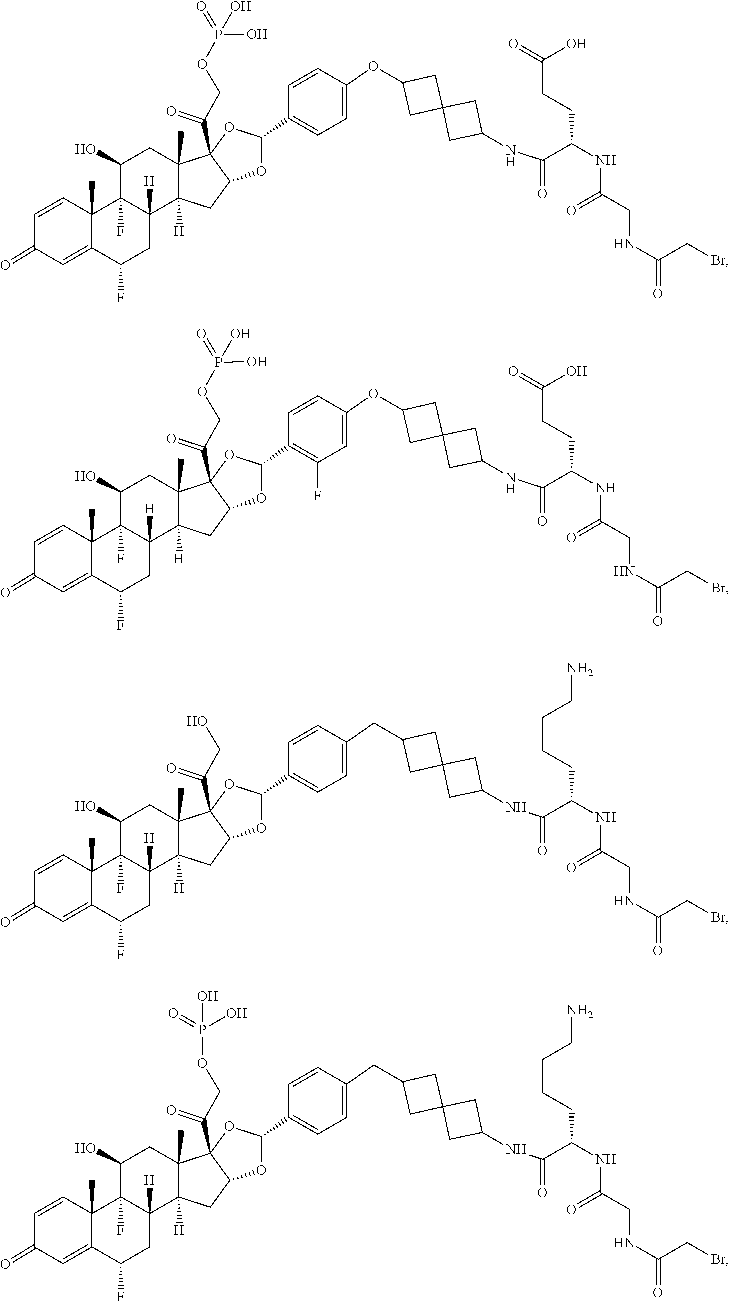 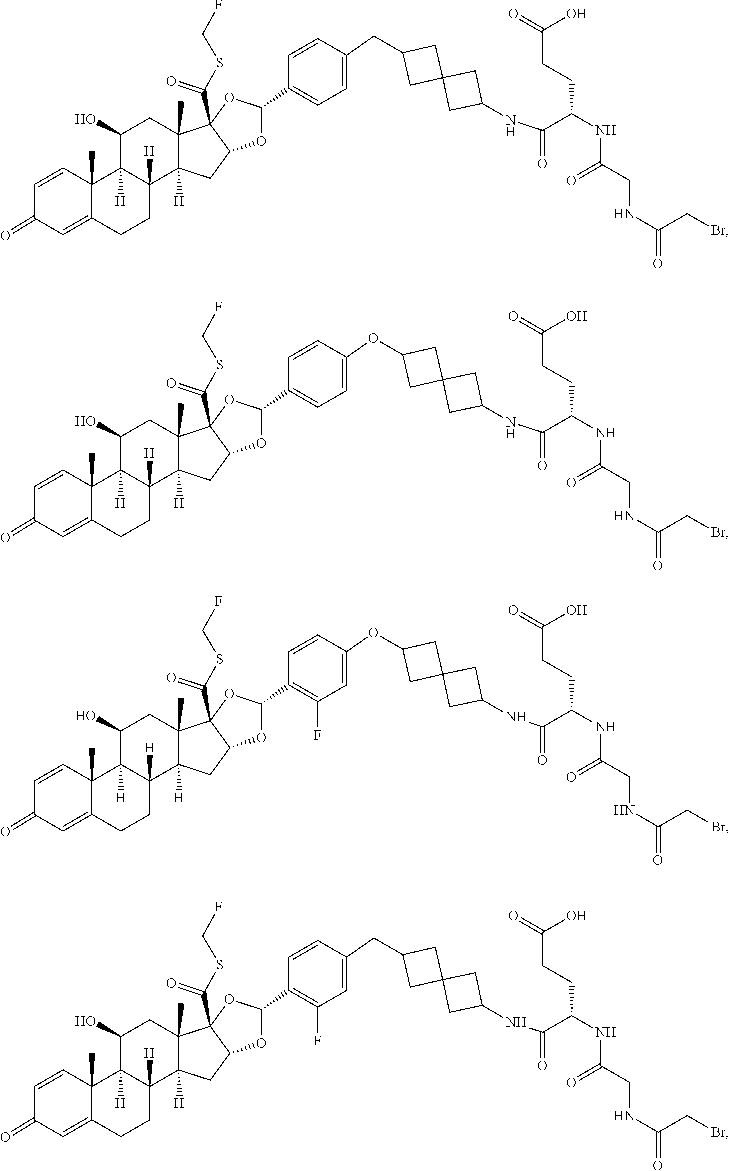 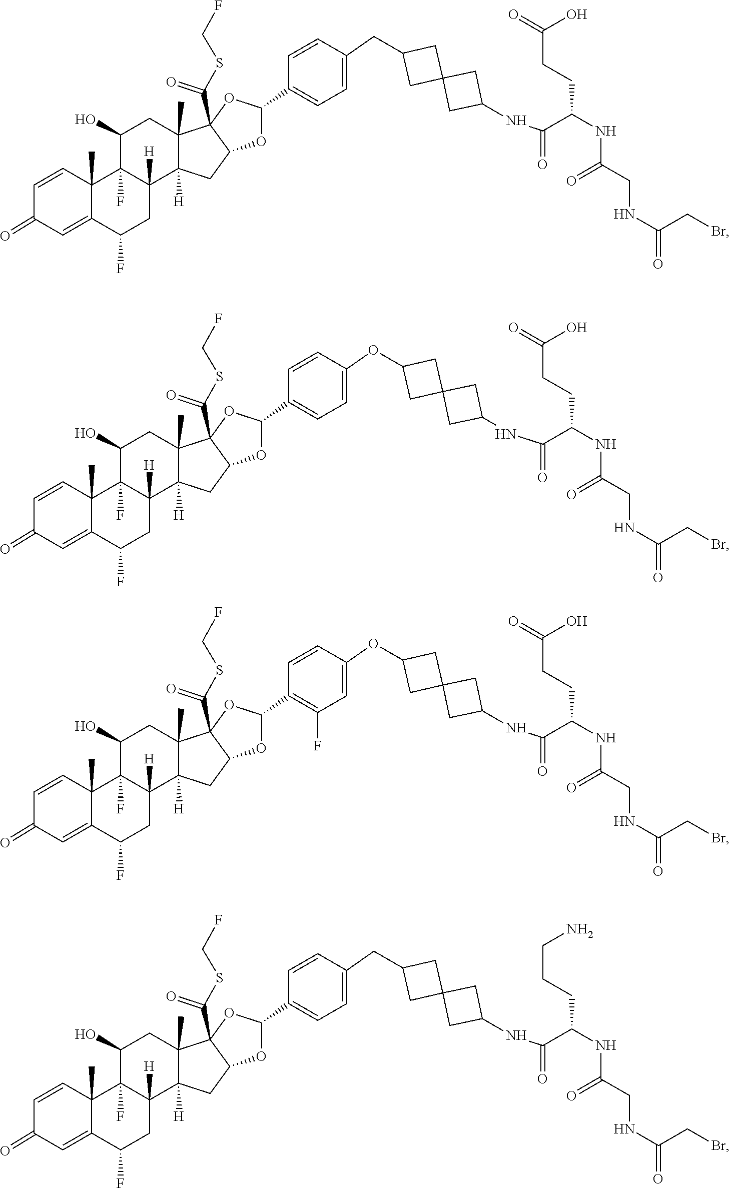 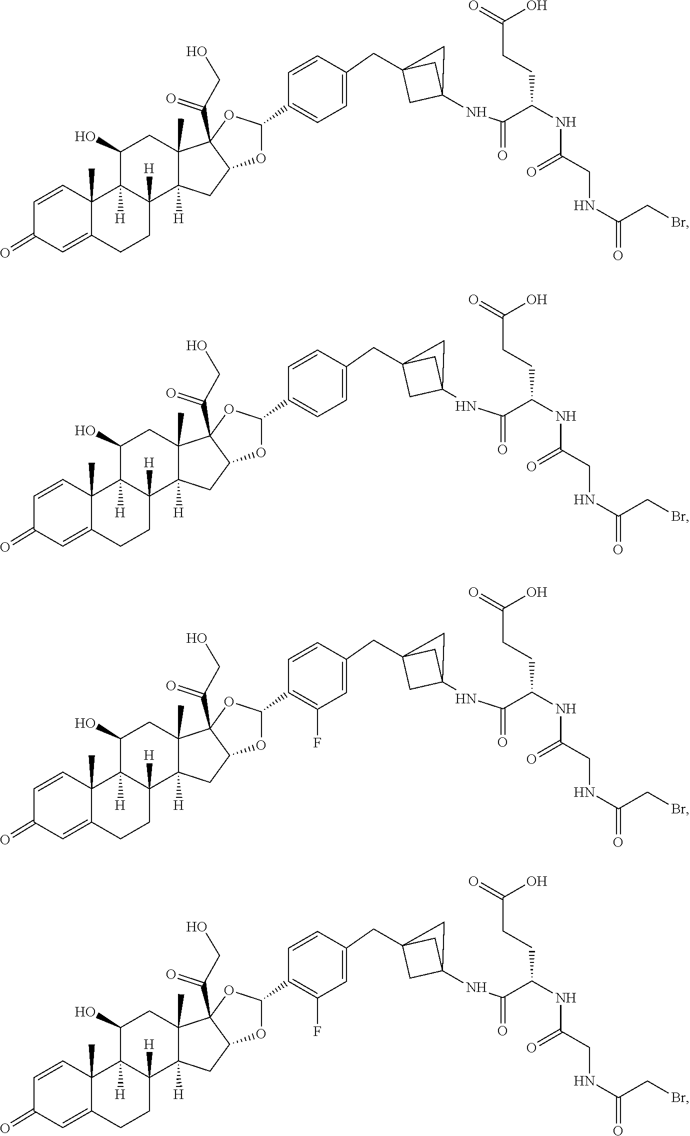 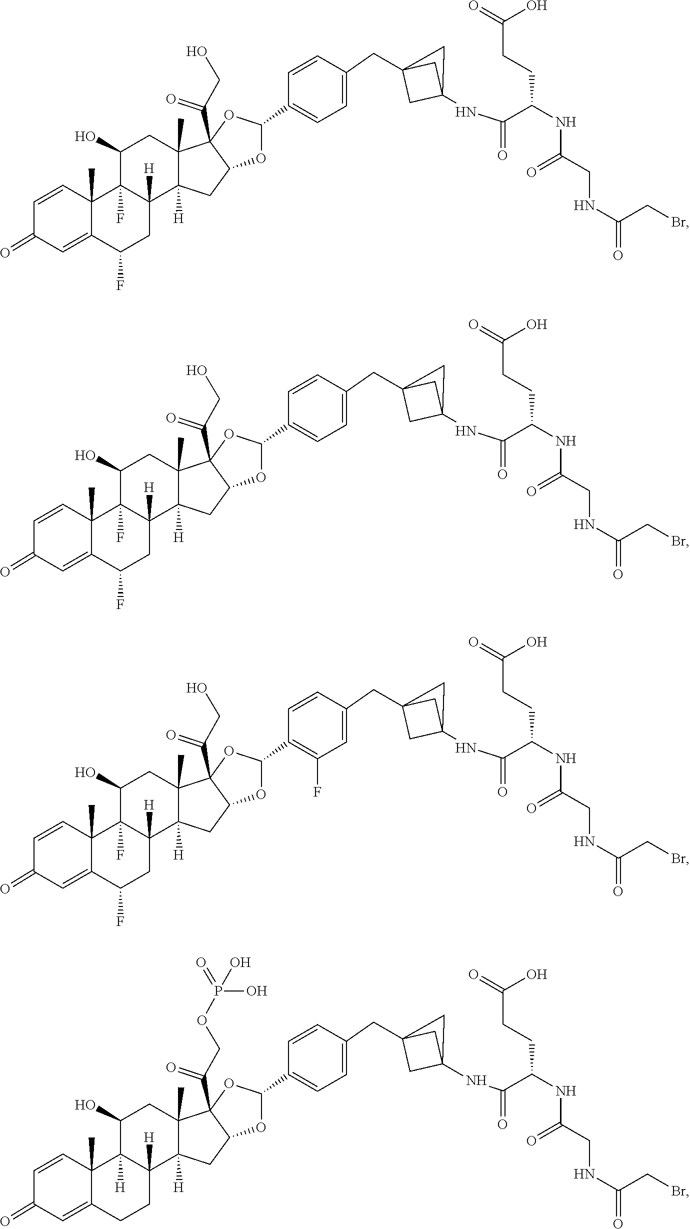 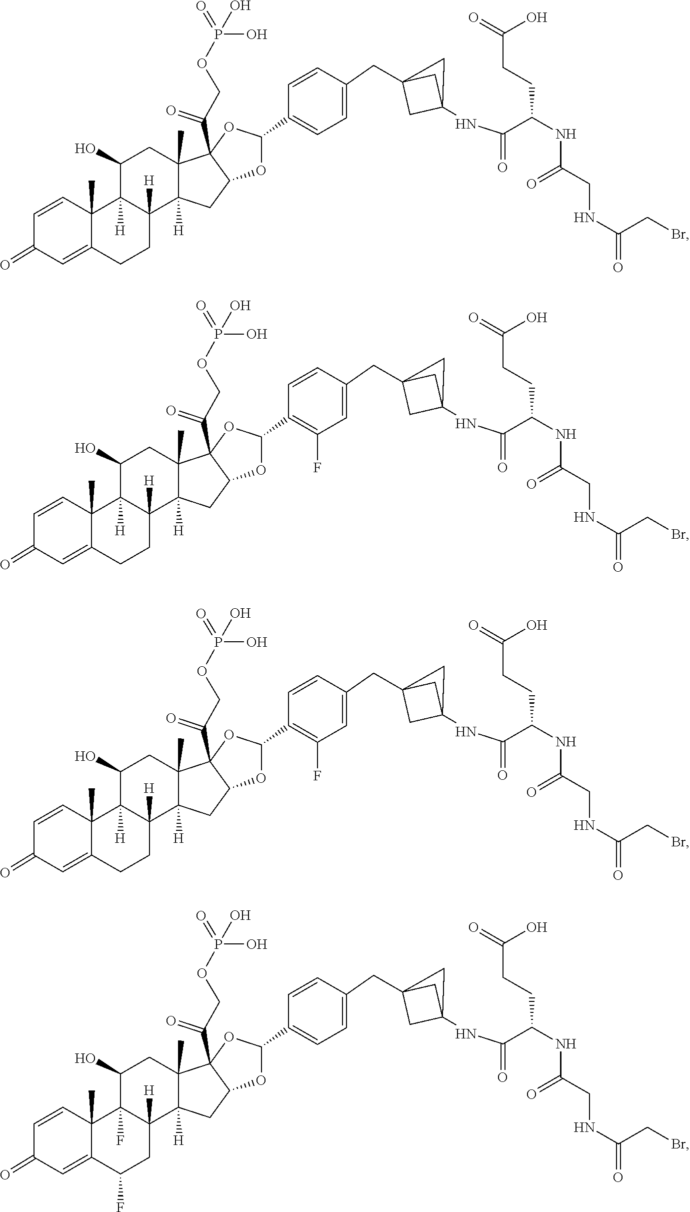 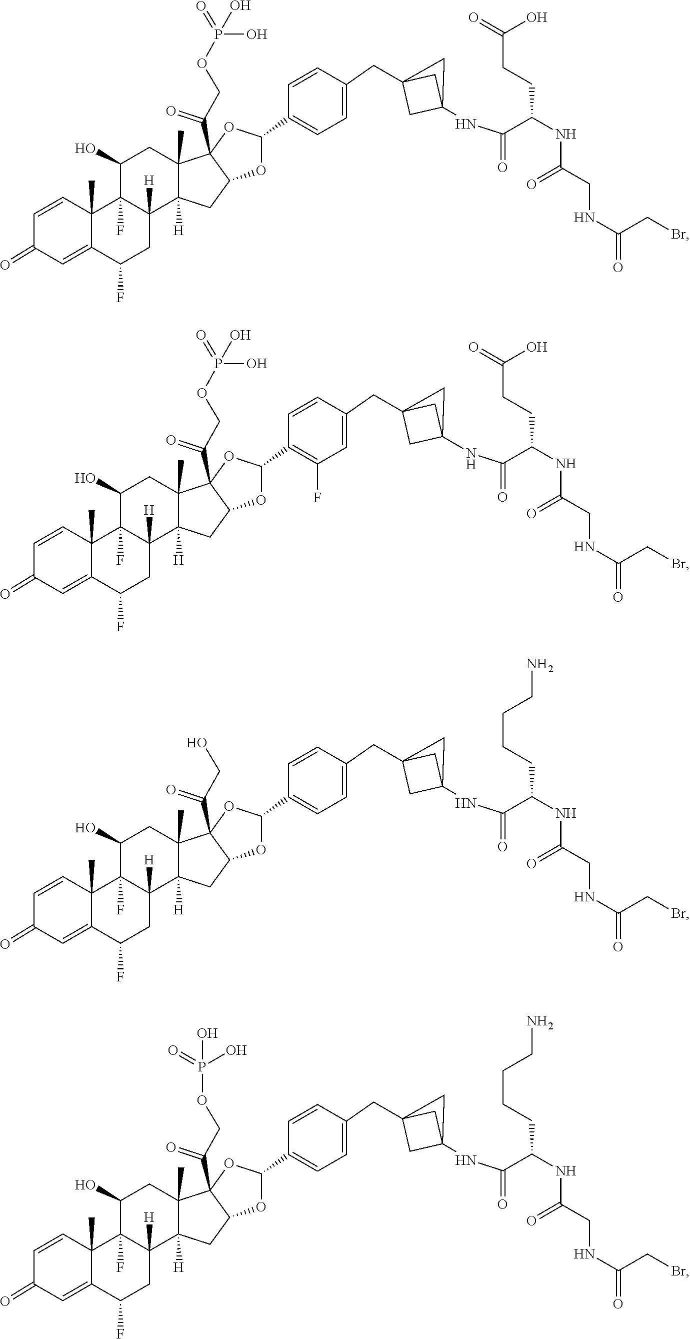 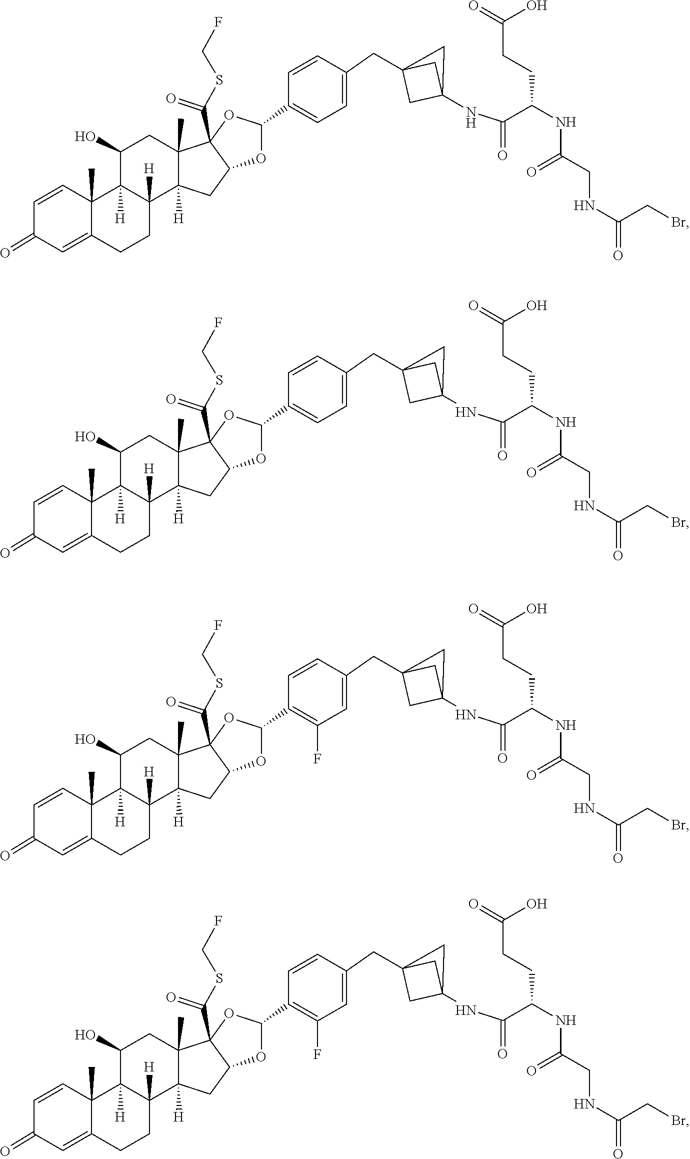 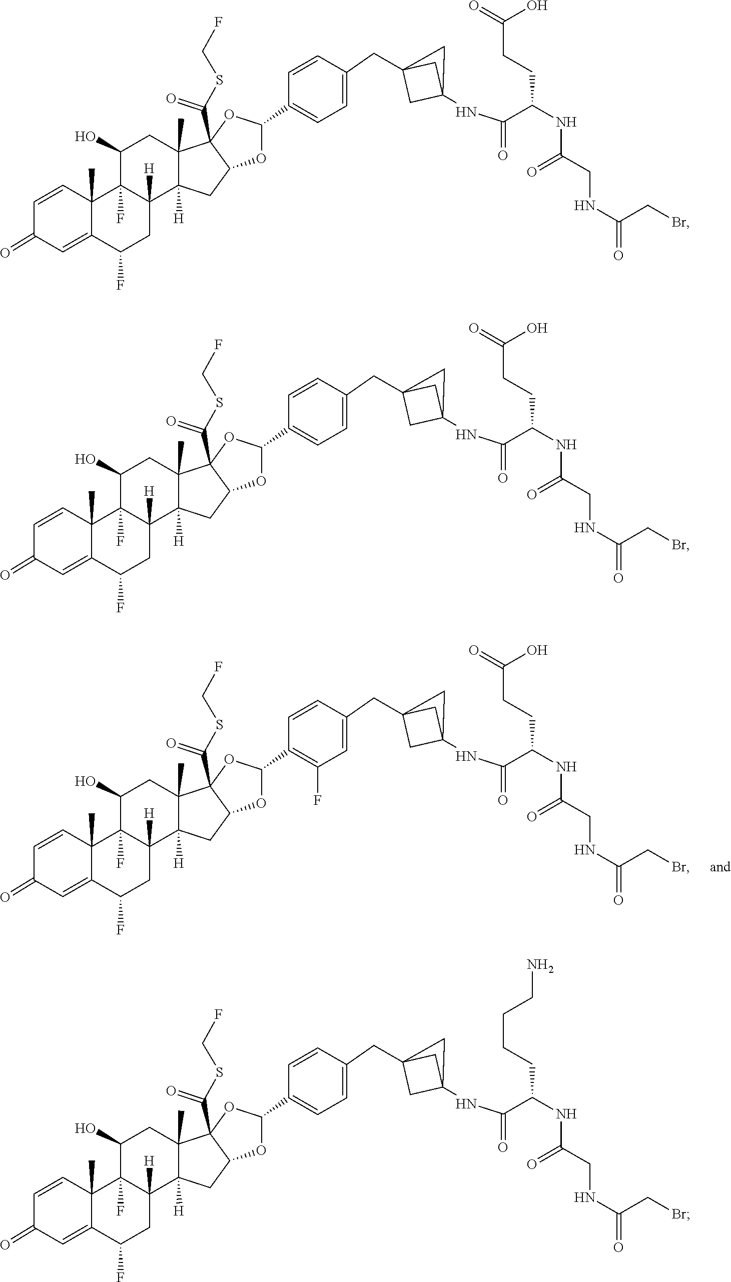
(h) a compound according to claim 133, embodiment (B), (C) or (D), wherein X or Z may be spiro[3.3]heptane or [1.1.1]bicyclopentane and Y may be CH₂ or O;
(i) said glucocorticoid agonist compound according to claim 133 or any of the foregoing is directly or indirectly attached to at least one cleavable or non-cleavable peptide and/or non-peptide linker (i.e., “steroid-linker payload” or “glucocorticosteroid-linker payload”);
(j) said glucocorticoid agonist compound according to claim 133 or any of the foregoing comprises at least one cleavable or non-cleavable linker (“L”), (“steroid-linker payload”), optionally “Q” which is a heterobifunctional group” or “heterotrifunctional group” which is a chemical moiety optionally used to connect the linker in the compound to an antibody or antibody fragment and at least one anti-inflammatory agent, (“AI”), wherein AI is a glucocorticoid agonist compound according to claim 133 or any of the foregoing which may be represented by the following structure:
Q-L-AI or AI-L-Q; optionally wherein the linker is selected from any of those disclosed herein or shown in the steroid-linker compounds in FIG. 118A-E; or
said at least one cleavable or non-cleavable linker selected from PAB and/or an amino acid or a peptide, optionally 1-12 amino acids, further optionally dipeptide, a tripeptide, a quatrapeptide, a pentapeptide and further optionally Gly, Asn, Asp, Gln, Leu, Lys, Ala, Phe, Cit, Val, Val-Cit, Val-Ala, Val-Gly, Val-Gln, Ala-Val, Cit-Cit, Lys-Val-Cit, Asp-Val-Ala, Ala-Ala-Asn, Asp-Val-Ala, Ala-Val-Cit, Ala-Asn-Val, betaAla-Leu-Ala-Leu, Lys-Val-Ala, Val-Leu-Lys, Asp-Val-Cit, Val-Ala-Val, and Ala-Ala-Asn; or optionally at least one of GlcA, PAB, and Glu-Gly; or
(k) a steroid-linker payload according to (j) which comprises at least one glucocorticoid agonist compound according to any one of the foregoing and at least one cleavable linker, and/or an immolative linker, which is directly or indirectly attached to the glucocorticoid agonist steroid compound; or
(l) said glucocorticoid agonist compound or steroid-linker payload according to claim 133 or any one of the foregoing is selected from any of the glucocorticoid agonist compounds or steroid-linker payload compounds disclosed in Example 3 or FIG. 118A-O excluding INX J and INX L; or
(m) said steroid-linker payload according to claim 133 or any of the foregoing is selected from:
(i) INX-SM-3-GluGly-Alkoxyamine, INX-SM-4-GluGly-Alkoxyamine, INX-SM-53-GluGly-Alkoxyamine, INX-SM-54-GluGly-Alkoxyamine, INX-SM-56-GluGly-Alkoxyamine, INX-SM-98-GluGly-Alkoxyamine, INX-SM-6-GluGly-Alkoxyamine, INX-SM-2-GluGly-Alkoxyamine, INX-SM-57-GluGly-Alkoxyamine, INX-SM-31-GluGly-Alkoxyamine, INX-SM-32-GluGly-Alkoxyamine, INX-SM-10-GluGly-Alkoxyamine, INX-SM-40-GluGly-Alkoxyamine, INX-SM-34-GluGly-Alkoxyamine, INX-SM-28-GluGly-Alkoxyamine, INX-SM-27-GluGly-Alkoxyamine, INX-SM-35-GluGly-Alkoxyamine, INX-SM-8-GluGly-Alkoxyamine, INX-SM-7-GluGly-Alkoxyamine, INX-SM-33-GluGly-Alkoxyamine or an glucocorticoid agonist (Payload)-linker conjugate wherein the Glu-Gly is substituted with a different cleavable peptide linker and/or wherein another INX or INX-SM payload is substituted for the INX-SM payload comprised therein optionally selected from those in FIG. 118A-O or one according to Formula I, II or III; or
(ii) INX-SM-53-GluGly-Bromoacetyl, INX-SM-3-GluGly-Bromoacetyl, INX-SM-54-GluGly-Bromoacetyl, INX-SM-1-GluGly-Bromoacetyl, INX-SM-4-GluGly-Bromoacetyl, INX-SM-2-GluGly-Bromoacetyl, INX-SM-47-GluGly-Bromoacetyl, INX-SM-7-GluGly-Bromoacetyl, INX-SM-8-GluGly-Bromoacetyl, INX-SM-56-GluGly-Bromoacetyl, INX-SM-32-GluGly-Bromoacetyl, INX-SM-6-GluGly-Bromoacetyl, INX-SM-10-GluGly-Bromoacetyl, INX-SM-33-GluGly-Bromoacetyl, INX-SM-31-GluGly-Bromoacetyl, INX-SM-35-GluGly-Bromoacetyl, INX-SM-9-GluGly-Bromoacetyl, INX-SM-28-GluGly-Bromoacetyl, INX-SM-27-GluGly-Bromoacetyl, INX-SM-34-GluGly-Bromoacetyl, INX-SM-35-GluGly-Bromoacetyl, INX-SM-40-GluGly-Bromoacetyl or an glucocorticoid agonist (Payload)-linker conjugate wherein the Glu-Gly is substituted with a different cleavable peptide linker and/or wherein another INX or INX-SM payload is substituted for the INX-SM payload comprised therein optionally selected from those in FIG. 118A-O or one according to Formula I, II or III;
(iii) INX-SM-53-GluGly-Dibenzocyclooctyne, INX-SM-1-GluGly-Dibenzocyclooctyne, INX-SM-4-GluGly-Dibenzocyclooctyne, INX-SM-54-GluGly-Dibenzocyclooctyne, INX-SM-7-GluGly-Dibenzocyclooctyne, INX-SM-8-GluGly-Dibenzocyclooctyne, INX-SM-2-GluGly-Dibenzocyclooctyne, INX-SM-57-GluGly-Dibenzocyclooctyne, INX-SM-40-GluGly-Dibenzocyclooctyne, INX-SM-34-GluGly-Dibenzocyclooctyne, INX-SM-28-GluGly-Dibenzocyclooctyne, INX-SM-27-GluGly-Dibenzocyclooctyne, INX-SM-35-GluGly-Dibenzocyclooctyne, INX-SM-9-GluGly-Dibenzocyclooctyne, INX-SM-10-GluGly-Dibenzocyclooctyne, INX-SM-31-GluGly-Dibenzocyclooctyne, INX-SM-32-GluGly-Dibenzocyclooctyne, INX-SM-33-GluGly-Dibenzocyclooctyne, INX-SM-56-GluGly-Dibenzocyclooctyne, INX-SM-6-GluGly-Dibenzocyclooctyne, INX-SM-3-GluGly-Dibenzocyclooctyne or an glucocorticoid agonist (Payload)-linker conjugate wherein the GluGly is substituted with a different cleavable peptide linker wherein another INX or INX-SM payload is substituted for the INX-SM payload comprised therein optionally selected from those in FIG. 118A-O or one according to Formula I, II or III; or
(iv) INX-SM-1-GluGly-NHS ester; INX-SM-31-GluGly-NHS ester; INX-SM-32-GluGly-NHS ester; INX-SM-33-GluGly-NHS ester; INX-SM-53-GluGly-NHS ester; INX-SM-7-GluGly-NHS ester; INX-SM-8-GluGly-NHS ester; INX-SM-2-GluGly-NHS ester; INX-SM-56-GluGly-NHS ester; INX-SM-6-GluGly-NHS ester; INX-SM-54-GluGly-NHS ester; INX-SM-4-GluGly-NHS ester; INX-SM-53-GluGly-NHS ester; INX-SM-3-GluGly-NHS ester; INX-SM-9-GluGly-NHS ester; INX-SM-40-GluGly-NHS ester; INX-SM-34-GluGly-NHS ester; INX-SM-28-GluGly-NHS ester; INX-SM-34-GluGly-NHS ester; INX-SM-28-GluGly-NHS ester; INX-SM-27-GluGly-NHS ester; INX-SM-35-GluGly-NHS ester; INX-SM-10-GluGly-NHS ester or an glucocorticoid agonist (Payload)-linker conjugate wherein the GluGly is substituted with a different cleavable peptide linker wherein another INX or INX-SM payload is substituted for the INX-SM payload comprised therein optionally selected from those in FIG. 118A-O or one according to Formula I, II or III;
(v) INX-SM-1-GluGly-Maleimide, INX-SM-3-GluGly-Maleimide, INX-SM-4-GluGly-Maleimide, INX-SM-8-GluGly-Maleimide, INX-SM-2-GluGly-Maleimide, INX-SM-7-GluGly-Maleimide, INX-SM-56-GluGly-Maleimide, INX-SM-6-GluGly-Maleimide, INX-SM-54-GluGly-Maleimide, INX-SM-53-GluGly-Maleimide, INX-SM-33-GluGly-Maleimide, INX-SM-35-GluGly-Maleimide, INX-SM-40-GluGly-Maleimide, INX-SM-34-GluGly-Maleimide, INX-SM-28-GluGly-Maleimide, INX-SM-27-GluGly-Maleimide, INX-SM-35-GluGly-Maleimide, INX-SM-9-GluGly-Maleimide, INX-SM-10-GluGly-Maleimide, INX-SM-31-GluGly-Maleimide, INX-SM-32-GluGly-Maleimide, INX-SM-57-GluGly-Maleimide or an glucocorticoid agonist (Payload)-linker conjugate wherein the GluGly is substituted with a different cleavable peptide linker wherein another INX or INX-SM payload is substituted for the INX-SM payload comprised therein optionally selected from those in FIG. 118A-O or one according to Formula I, II or III; or
(vi) INX-SM-3-GluGly-Tetrazine, INX-SM-53-GluGly-Tetrazine, INX-SM-1-GluGly-Tetrazine, INX-SM-54-GluGly-Tetrazine, INX-SM-6-GluGly-Tetrazine, INX-SM-56-GluGly-Tetrazine, INX-SM-4-GluGly-Tetrazine, INX-SM-10-GluGly-Tetrazine, INX-SM-31-GluGly-Tetrazine, INX-SM-32-GluGly-Tetrazine, INX-SM-33-GluGly-Tetrazine, INX-SM-7-GluGly-Tetrazine, INX-SM-8-GluGly-Tetrazine, INX-SM-9-GluGly-Tetrazine, INX-SM-27-GluGly-Tetrazine, INX-SM-35-GluGly-Tetrazine, INX-SM-2-GluGly-Tetrazine, INX-SM-40-GluGly-Tetrazine, INX-SM-34-GluGly-Tetrazine, INX-SM-28-GluGly-Tetrazine, INX-SM-27-GluGly-Tetrazine or an glucocorticoid agonist (Payload)-linker conjugate wherein the GluGly is substituted with a different cleavable peptide linker wherein another INX or INX-SM payload is substituted for the INX-SM payload comprised therein optionally selected from those in FIG. 118A-O or one according to Formula I, II or III; or
(vii) INX-SM-6-GluGly-Amine, INX-SM-54-GluGly-Amine, INX-SM-4-GluGly-Amine, INX-SM-53-GluGly-Amine, INX-SM-2-GluGly-Amine, INX-SM-56-GluGly-Amine, INX-SM-57-GluGly-Amine, INX-SM-35-GluGly-Amine, INX-SM-27-GluGly-Amine, INX-SM-40-GluGly-Amine, INX-SM-34-GluGly-Amine, INX-SM-28-GluGly-Amine, INX-SM-35-GluGly-Amine, INX-SM-9-GluGly-Amine, INX-SM-10-GluGly-Amine, INX-SM-31-GluGly-Amine, INX-SM-32-GluGly-Amine, INX-SM-33-GluGly-Amine, INX-SM-7-GluGly-Amine, INX-SM-8-GluGly-Amine, INX-SM-1-GluGly-Amine, INX-SM-3-GluGly-Amine or an glucocorticoid agonist (Payload)-linker conjugate wherein the GluGly is substituted with a different cleavable peptide linker wherein another INX or INX-SM payload is substituted for the INX-SM payload comprised therein optionally selected from those in FIG. 118A-O or one according to Formula I, II or III; or
(viii) INX-SM-53-PAB-GluGly-Alkoxyamine, INX-SM-1-PAB-GluGly-Alkoxyamine, INX-SM-3-PAB-GluGly-Alkoxyamine, INX-SM-2-PAB-GluGly-Alkoxyamine, INX-SM-56-PAB-GluGly-Alkoxyamine, INX-SM-35-PAB-GluGly-Alkoxyamine, INX-SM-25-PAB-GluGly-Alkoxyamine, INX-SM-27-PAB-GluGly-Alkoxyamine, INX-SM-35-PAB-GluGly-Alkoxyamine, INX-SM-9-PAB-GluGly-Alkoxyamine, INX-SM-10-PAB-GluGly-Alkoxyamine, INX-SM-31-PAB-GluGly-Alkoxyamine, INX-SM-32-PAB-GluGly-Alkoxyamine, INX-SM-33-PAB-GluGly-Alkoxyamine, INX-SM-57-PAB-GluGly-Alkoxyamine, INX-SM-7-PAB-GluGly-Alkoxyamine, INX-SM-8-PAB-GluGly-Alkoxyamine, INX-SM-6-PAB-GluGly-Alkoxyamine, INX-SM-54-PAB-GluGly-Alkoxyamine, INX-SM-4-PAB-GluGly-Alkoxyamine, INX-SM-40-PAB-GluGly-Alkoxyamine, INX-SM-34-PAB-GluGly-Alkoxyamine or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O or one according to Formula I, II or III; or
(ix) INX-SM-1-PAB-GluGly-Bromoacetyl, INX-SM-3-PAB-GluGly-Bromoacetyl, INX-SM-2-PAB-GluGly-Bromoacetyl, INX-SM-7-PAB-GluGly-Bromoacetyl, INX-SM-8-PAB-GluGly-Bromoacetyl, INX-SM-40-PAB-GluGly-Bromoacetyl, INX-SM-56-PAB-GluGly-Bromoacetyl, INX-SM-6-PAB-GluGly-Bromoacetyl, INX-SM-154PAB-GluGly-Bromoacetyl, INX-SM-4-PAB-GluGly-Bromoacetyl, INX-SM-33-PAB-GluGly-Bromoacetyl, INX-PAB-GluGly-Bromoacetyl, INX-SM-32-PAB-GluGly-Bromoacetyl, INX-SM-10-PAB-GluGly-Bromoacetyl, INX-SM-34-PAB-GluGly-Bromoacetyl, INX-SM-31-PAB-GluGly-Bromoacetyl, INX-SM-9-PAB-GluGly-Bromoacetyl, INX-SM-28-PAB-GluGly-Bromoacetyl, INX-SM-27-PAB-GluGly-Bromoacetyl, INX-SM-35-PAB-GluGly-Bromoacetyl, INX-SM-53-PAB-GluGly-Bromoacetyl or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O or one according to Formula I, II or III;
(x) INX-SM-6-PAB-GluGly-Dibenzocyclooctyne, INX-SM-54-PAB-GluGly-Dibenzocyclooctyne, INX-SM-4-PAB-GluGly-Dibenzocyclooctyne, INX-SM-53-PAB-GluGly-Dibenzocyclooctyne, INX-SM-1-PAB-GluGly-Dibenzocyclooctyne, INX-SM-7-PAB-GluGly-Dibenzocyclooctyne, INX-SM-8-PAB-GluGly-Dibenzocyclooctyne, INX-SM-2-PAB-GluGly-Dibenzocyclooctyne, INX-SM-56-PAB-GluGly-Dibenzocyclooctyne, INX-SM-57-PAB-GluGly-Dibenzocyclooctyne, INX-SM-33-PAB-GluGly-Dibenzocyclooctyne, INX-SM-32-PAB-GluGly-Dibenzocyclooctyne, INX-SM-31-PAB-GluGly-Dibenzocyclooctyne, INX-SM-3-PAB-GluGly-Dibenzocyclooctyne, INX-SM-9-PAB-GluGly-Dibenzocyclooctyne, INX-SM-27-PAB-GluGly-Dibenzocyclooctyne, INX-SM-35-PAB-GluGly-Dibenzocyclooctyne, INX-SM-34-PAB-GluGly-Dibenzocyclooctyne, INX-SM-28-PAB-GluGly-Dibenzocyclooctyne, INX-SM-40-PAB-GluGly-ibenzocyclooctyne, INX-SM-10-PAB-GluGly-Dibenzocyclooctyne or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O or one according to Formula I, II or III; or
(xi) INX-SM-56-PAB-GluGly-NHS ester, INX-SM-54-PAB-GluGly-NHS ester, INX-SM-4-PAB-GluGly-NHS ester, INX-SM-53-PAB-GluGly-NHS ester, INX-SM-1-PAB-GluGly-NHS ester, INX-SM-3-PAB-GluGly-NHS ester, INX-SM-33-PAB-GluGly-NHS ester, INX-SM-57-PAB-GluGly-NHS ester, INX-SM-7-PAB-GluGly-NHS ester, INX-SM-8-PAB-GluGly-NHS ester, INX-SM-27-PAB-GluGly-NHS ester, INX-SM-35-PAB-GluGly-NHS ester, INX-SM-9-PAB-GluGly-NHS ester, INX-SM-10-PAB-GluGly-NHS ester, INX-SM-31-PAB-GluGly-NHS ester, INX-SM-32-PAB-GluGly-NHS ester, INX-SM-40-PAB-GluGly-NHS ester, INX-SM-34-PAB-GluGly-NHS ester, INX-SM-28-PAB-GluGly-NHS ester, INX-SM-2-PAB-GluGly-NHS ester or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O or one according to Formula I, II or III; or
(xii) INX-SM-1-PAB-GluGly-Maleimide, INX-SM-53-PAB-GluGly-Maleimide, INX-SM-5-PAB-GluGly-Maleimide, INX-SM-2-PAB-GluGly-Maleimide, INX-SM-8-PAB-GluGly-Maleimide, INX-SM-56-PAB-GluGly-Maleimide, INX-SM-54-PAB-GluGly-Maleimide, INX-SM-4-PAB-GluGly-Maleimide, INX-SM-57-PAB-GluGly-Maleimide, INX-SM-7-PAB-GluGly-Maleimide, INX-SM-32-PAB-GluGly-Maleimide, INX-SM-31-PAB-GluGly-Maleimide, INX-SM-53-PAB-GluGly-Maleimide, INX-SM-3-PAB-GluGly-Maleimide, INX-SM-34-PAB-GluGly-Maleimide, INX-SM-28-PAB-GluGly-Maleimide, INX-SM-40-PAB-GluGly-Maleimide, INX-SM-27-PAB-GluGly-Maleimide, INX-SM-35-PAB-GluGly-Maleimide, INX-SM-9-PAB-GluGly-Maleimide, INX-SM-10-PAB-GluGly-Maleimide or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O or one according to Formula I, II or III; or
(xiii) INX-SM-6-PAB-GluGly-Tetrazine, INX-SM-54-PAB-GluGly-Tetrazine, INX-SM-4-PAB-GluGly-Tetrazine, INX-SM-53-PAB-GluGly-Tetrazine, INX-SM-1-PAB-GluGly-Tetrazine, INX-SM-3-PAB-GluGly-Tetrazine, INX-SM-57-PAB-GluGly-Tetrazine, INX-SM-7-PAB-GluGly-Tetrazine, INX-SM-8-PAB-GluGly-Tetrazine, INX-SM-2-PAB-GluGly-Tetrazine, INX-SM-31-PAB-GluGly-Tetrazine, INX-SM-32-PAB-GluGly-Tetrazine, INX-SM-33-PAB-GluGly-Tetrazine, INX-SM-56-PAB-GluGly-Tetrazine, INX-SM-35-PAB-GluGly-Tetrazine, INX-SM-9-PAB-GluGly-Tetrazine, INX-SM-40-PAB-GluGly-Tetrazine, INX-SM-34-PAB-GluGly-Tetrazine, INX-SM-28-PAB-GluGly-Tetrazine, INX-SM-27-PAB-GluGly-Tetrazine, INX-SM-35-PAB-GluGly-Tetrazine, INX-SM-10-PAB-GluGly-Tetrazine or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O or one according to Formula I, II or III; or
(xiv) INX-SM-1-PAB-GluGly-Amine, INX-SM-3-PAB-GluGly-Amine, INX-SM-8-PAB-GluGly-Amine, INX-SM-2-PAB-GluGly-Amine, INX-SM-56-PAB-GluGly-Amine, INX-SM-6-PAB-GluGly- or one according to Formula I, II or III Amine, INX-SM-54-PAB-GluGly-Amine, INX-SM-4-PAB-GluGly-Amine, INX-SM-53-PAB-GluGly-Amine, INX-SM-33-PAB-GluGly-Amine, INX-SM-53-PAB-GluGly-Amine, INX-SM-7-PAB-GluGly-Amine, INX-SM-9-PAB-GluGly-Amine, INX-SM-35-PAB-GluGly-Amine, INX-SM-40-PAB-GluGly-Amine, INX-SM-34-PAB-GluGly-Amine, INX-SM-28-PAB-GluGly-Amine, INX-SM-27-PAB-GluGly-Amine, INX-SM-35-PAB-GluGly-Amine, INX-SM-10-PAB-GluGly-Amine, INX-SM-31-PAB-GluGly-Amine, INX-SM-32-PAB-GluGly-Amine or another glucocorticoid agonist (Payload)-linker conjugate wherein the GluGly and/or the PAB is substituted with a different cleavable peptide or non-peptide linker wherein another INX or INX-SM payload is substituted for the INX-SM payload comprised therein optionally selected from those in FIG. 118A-O or one according to Formula I, II or III; or
(xv) INX-SM-1-PAB-GlcA-Alkoxyamine, INX-SM-35-PAB-GlcA-Alkoxyamine, INX-SM-9-PAB-GlcA-Alkoxyamine, INX-SM-10-PAB-GlcA-Alkoxyamine, INX-SM-54-PAB-GlcA-Alkoxyamine, INX-SM-31-PAB-GlcA-Alkoxyamine, INX-SM-32-PAB-GlcA-Alkoxyamine, INX-SM-33-PAB-GlcA-Alkoxyamine, INX-SM-57-PAB-GlcA-Alkoxyamine, INX-SM-7-PAB-GlcA-Alkoxyamine, INX-SM-8-PAB-GlcA-Alkoxyamine, INX-SM-2-PAB-GlcA-Alkoxyamine, INX-SM-56-PAB-GlcA-Alkoxyamine, INX-SM-6-PAB-GlcA-Alkoxyamine, INX-SM-4-PAB-GlcA-Alkoxyamine, INX-SM-53-PAB-GlcA-Alkoxyamine, INX-SM-27-PAB-GlcA-Alkoxyamine, INX-SM-40-PAB-GlcA-Alkoxyamine, INX-SM-34-PAB-GlcA-Alkoxyamine, INX-SM-28-PAB-GlcA-Alkoxyamine, INX-SM-3-PAB-GlcA-Alkoxyamine or another glucocorticoid agonist (Payload)-linker conjugate wherein the GlcA and/or the PAB is substituted with a different cleavable peptide or non-peptide linker and/or another INX or INX-SM payload is substituted for the INX-SM payload comprised therein optionally selected from those in FIG. 118A-O or one according to Formula I, II or III; or
(xvi) INX-SM-3-PAB-GlcA-Bromoacetyl, INX-SM-4-PAB-GlcA-Bromoacetyl, INX-SM-56-PAB-GlcA-Bromoacetyl, INX-SM-54-PAB-GlcA-Bromoacetyl, INX-SM-4-PAB-GlcA-Bromoacetyl, INX-SM-53-PAB-GlcA-Bromoacetyl, INX-SM-7-PAB-GlcA-Bromoacetyl, INX-SM-8-PAB-GlcA-Bromoacetyl, INX-SM-2-PAB-GlcA-Bromoacetyl, INX-SM-40-PAB-GlcA-Bromoacetyl, INX-SM-57-PAB-GlcA-Bromoacetyl, INX-SM-33-PAB-GlcA-Bromoacetyl, INX-SM-10-PAB-GlcA-Bromoacetyl, INX-SM-34-PAB-GlcA-Bromoacetyl, INX-SM-31-PAB-GlcA-Bromoacetyl, INX-SM-32-PAB-GlcA-Bromoacetyl, INX-SM-35-PAB-GlcA-Bromoacetyl, INX-SM-9-PAB-GlcA-Bromoacetyl, INX-SM-28-PAB-GlcA-Bromoacetyl, INX-SM-27-PAB-GlcA-Bromoacetyl, INX-SM-1-PAB-GlcA-Bromoacetyl or another glucocorticoid agonist (Payload)-linker conjugate wherein the GluGly and/or the PAB is substituted with a different cleavable peptide or non-peptide linker and/or another INX or INX-SM payload is substituted for the INX-SM payload comprised therein optionally selected from those in FIG. 118A-O or one according to Formula I, II or III; or
(xvii) INX-SM-4-PAB-GlcA-Dibenzocyclooctyne, INX-SM-54-PAB-GlcA-Dibenzocyclooctyne, INX-SM-1-PAB-GlcA-Dibenzocyclooctyne, INX-SM-54-PAB-GlcA-Dibenzocyclooctyne, INX-SM-33-PAB-GlcA-Dibenzocyclooctyne, INX-SM-57-PAB-GlcA-Dibenzocyclooctyne, INX-SM-7-PAB-GlcA-Dibenzocyclooctyne, INX-SM-8-PAB-GlcA-Dibenzocyclooctyne, INX-SM-2-PAB-GlcA-Dibenzocyclooctyne, INX-SM-5-PAB-GlcA-Dibenzocyclooctyne, INX-SM-6-PAB-GlcA-Dibenzocyclooctyne, INX-SM-35-PAB-GlcA-Dibenzocyclooctyne, INX-SM-9-PAB-GlcA-Dibenzocyclooctyne, INX-SM-10-PAB-GlcA-Dibenzocyclooctyne, INX-SM-31-PAB-GlcA-Dibenzocyclooctyne, INX-SM-32-PAB-GlcA-Dibenzocyclooctyne, INX-SM-27-PAB-GlcA-Dibenzocyclooctyne, INX-SM-35-PAB-GlcA-Dibenzocyclooctyne, INX-SM-28-PAB-GlcA-Dibenzocyclooctyne, INX-SM-34-PAB-GlcA-Dibenzocyclooctyne, INX-SM-40-PAB-GlcA-Dibenzocyclooctyne, INX-SM-3-PAB-GlcA-Dibenzocyclooctyne or another glucocorticoid agonist (Payload)-linker conjugate wherein the GlcA and/or the PAB linker is substituted with a different cleavable peptide or non-peptide linker and/or another INX or INX-SM payload is substituted for the INX-SM payload comprised therein optionally selected from those in FIG. 118A-O or one according to Formula I, II or III; or
(xviii) INX-SM-3-PAB-GlcA-NHS Ester, INX-SM-53-PAB-GlcA-NHS Ester, INX-SM-4-PAB-GlcA-NHS Ester, INX-SM-56-PAB-GlcA-NHS Ester, INX-SM-54-PAB-GlcA-NHS Ester, INX-SM-8-PAB-GlcA-NHS Ester, INX-SM-2-PAB-GlcA-NHS Ester, INX-SM-7-PAB-GlcA-NHS Ester, INX-SM-57-PAB-GlcA-NHS Ester, INX-SM-32-PAB-GlcA-NHS Ester, INX-SM-33-PAB-GlcA-NHS Ester, INX-SM-31-PAB-GlcA-NHS Ester, INX-SM-9-PAB-GlcA-NHS Ester, INX-SM-10-PAB-GlcA-NHS Ester, INX-SM-35-PAB-GlcA-NHS Ester, INX-SM-27-PAB-GlcA-NHS Ester, INX-SM-28-PAB-GlcA-NHS Ester, INX-SM-40-PAB-GlcA-NHS Ester, INX-SM-34-PAB-GlcA-NHS Ester, INX-SM-1-PAB-GlcA-NHS Ester or another glucocorticoid agonist (Payload)-linker conjugate wherein the GlcA and/or the PAB is substituted with a different cleavable peptide or non-peptide linker and/or another INX or INX-SM payload is substituted for the INX-SM payload comprised therein optionally selected from those in FIG. 118A-O or one according to Formula I, II or III; or
(xix) INX-SM-3-PAB-GlcA-Maleimide, INX-SM-4-PAB-GlcA-Maleimide, INX-SM-53-PAB-GlcA-Maleimide, INX-SM-31-PAB-GlcA-Maleimide, INX-SM-32-PAB-GlcA-Maleimide, INX-SM-33-PAB-GlcA-Maleimide, INX-SM-53-PAB-GlcA-Maleimide, INX-SM-7-PAB-GlcA-Maleimide, INX-SM-8-PAB-GlcA-Maleimide, INX-SM-2-PAB-GlcA-Maleimide, INX-SM-56-PAB-GlcA-Maleimide, INX-SM-6-PAB-GlcA-Maleimide, INX-SM-54-PAB-GlcA-Maleimide, INX-SM-1-PAB-GlcA-Maleimide, INX-SM-9-PAB-GlcA-Maleimide, INX-SM-35-PAB-GlcA-Maleimide, INX-SM-27-PAB-GlcA-Maleimide, INX-SM-28-PAB-GlcA-Maleimide, INX-SM-34-PAB-GlcA-Maleimide, INX-SM-40-PAB-GlcA-Maleimide, INX-SM-10-PAB-GlcA-Maleimide or another glucocorticoid agonist (Payload)-linker conjugate wherein the GlcA and/or the PAB is substituted with a different cleavable peptide or non-peptide linker and/or another INX or INX-SM payload is substituted for the INX-SM payload comprised therein optionally selected from those in FIG. 118A-O or one according to Formula I, II or III; or
(xx) INX-SM-33-PAB-GlcA-Tetrazine, INX-SM-57-PAB-GlcA-Tetrazine, INX-SM-7-PAB-GlcA-Tetrazine, INX-SM-8-PAB-GlcA-Tetrazine, INX-SM-2-PAB-GlcA-Tetrazine, INX-SM-56-PAB-GlcA-Tetrazine, INX-SM-6-PAB-GlcA-Tetrazine, INX-SM-54-PAB-GlcA-Tetrazine, INX-SM-4-PAB-GlcA-Tetrazine, INX-SM-9-PAB-GlcA-Tetrazine, INX-SM-35-PAB-GlcA-Tetrazine, INX-SM-27-PAB-GlcA-Tetrazine, INX-SM-28-PAB-GlcA-Tetrazine, INX-SM-34-PAB-GlcA-Tetrazine, INX-SM-40-PAB-GlcA-Tetrazine, INX-SM-10-PAB-GlcA-Tetrazine or another glucocorticoid agonist (Payload)-linker conjugate wherein the GlcAy and/or the PAB linker is substituted with a different cleavable peptide or non-peptide linker and/or another INX or INX-SM payload is substituted for the INX-SM payload comprised therein optionally selected from those in FIG. 118A-O or one according to Formula I, II or III; or
(xxi) INX-SM-1-PAB-GlcA-Amine, INX-SM-3-PAB-GlcA-Amine, INX-SM-53-PAB-GlcA-Amine, INX-SM-6-PAB-GlcA-Amine, INX-SM-54-PAB-GlcA-Amine, INX-SM-8-PAB-GlcA-Amine, INX-SM-2-PAB-GlcA-Amine, INX-SM-56-PAB-GlcA-Amine, INX-SM-4-PAB-GlcA-Amine, INX-SM-35-PAB-GlcA-Amine, INX-SM-8-PAB-GlcA-Amine, INX-SM-10-PAB-GlcA-Amine, INX-SM-31-PAB-GlcA-Amine, INX-SM-32-PAB-GlcA-Amine, INX-SM-33-PAB-GlcA-Amine, INX-SM-57-PAB-GlcA-Amine, INX-SM-27-PAB-GlcA-Amine, INX-SM-35-PAB-GlcA-Amine, INX-SM-34-PAB-GlcA-Amine, INX-SM-28-PAB-GlcA-Amine, INX-SM-40-PAB-GlcA-Amine, INX-SM-7-PAB-GlcA-Amine or another glucocorticoid agonist (Payload)-linker conjugate wherein the GlcA and/or the PAB linker is substituted with a different cleavable peptide or non-peptide linker and/or another INX or INX-SM payload is substituted for the INX-SM payload comprised therein optionally selected from those in FIG. 118A-O or one according to Formula I, II or III; or
(xxii) Alkoxyamine-GlcA-PAB-DMEDA-INX-SM3, or Alkoxyamine-GlyGlu-PAB-DMEDA-INX-SM3, or other INX linker payloads wherein INX-SM3 is substituted for a payload selected from those in FIG. 118A-O or another compound of Formula I, II or III and comprises the same or different peptide or non-peptide linkers and the linker is attached to the same or different INX steroid via the C11-OH;
(xxiii) Bromoacetyl-GlcA-PAB-DMEDA-INX-SM3, or Bromoacetyl-GlyGlu-PAB-DMEDA-INX-SM3, or other INX linker payloads wherein INX-SM3 is substituted for a payload selected from those in FIG. 118A-O or another compound of Formula I, II or III and/or comprises the same or different peptide or non-peptide linkers comprising the same or different peptide or non-peptide linkers wherein the linker is attached to the same or different INX steroid via the C11-OH;
(xxiv) Dibenzocyclooctyne-GlcA-PAB-DMEDA-INX-SM3, or Dibenzocyclooctyne-GlyGlu-PAB-DMEDA-INX-SM3, or other INX linker payloads wherein INX-SM3 is substituted for a payload selected from those in FIG. 118A-O or another compound of Formula I, II or III and/or comprises the same or different peptide or non-peptide linkers wherein the linker is attached to the same or different INX steroid via the C11-OH;
(xxv) Tetrazine-GlcA-PAB-DMEDA-INX-SM3, or Tetrazine-GlyGlu-PAB-DMEDA-INX-SM3, or other INX linker payloads wherein INX-SM3 is substituted for a payload selected from those in FIG. 118A-O or another compound of Formula I, II or III and/or comprises the same or different peptide or non-peptide linkers wherein the linker is attached to the same or different INX steroid via the C11-OH;
(xxvi) Alkoxyamine-GlcA-PAB-DMEDA-INX-SM3, or Alkoxyamine-GlyGlu-PAB-DMEDA-INX-SM3, or other INX linker payloads wherein INX-SM3 is substituted for a payload selected from those in FIG. 118A-O or another compound of Formula I, II or III and/or comprises the same or different linkers wherein a linker is attached to the same or different INX steroid payload via C17;
(xxvii) Bromoacetyl-GlcA-PAB-DMEDA-INX-SM3, or Bromoacetyl-GlyGlu-PAB-DMEDA-INX-SM3, or other INX linker payloads wherein INX-SM3 is substituted for a payload selected from those in FIG. 118A-O or another compound of Formula I, II or III and/or comprises the same or different INX steroid payload via C17;
(xxviii) Maleimide-GlcA-PAB-DMEDA-INX-SM3, or Maleimide-GlyGlu-PAB-DMEDA-INX-SM3, or other INX linker payloads wherein INX-SM3 is substituted for a payload selected from those in FIG. 118A-O or another compound of Formula I, II or III and/or comprises the same or different linker wherein the linker is attached to the same or different INX steroid payload via C17;
(xxix) Dibenzocyclooctyne-GlcA-PAB-DMEDA-INX-SM3, or Dibenzocyclooctyne-GlyGlu-PAB-DMEDA-INX-SM3, or other INX linker payloads wherein INX-SM3 is substituted for a payload selected from those in FIG. 118A-O or another compound of Formula I, II or III and/or comprises the same or different wherein the linker is attached to the same or different INX steroid payload via C17;
(xxx) Tetrazine-GlcA-PAB-DMEDA-INX-SM3, or Tetrazine-GlyGlu-PAB-DMEDA-INX-SM3, or other INX linker payloads wherein INX-SM3 is substituted for a payload selected from those in FIG. 118A-O or another compound of Formula I, II or III and/or comprises the same or different INX steroid payload via C17; and
(xxxi) Amine-GlcA-PAB-DMEDA-INX-SM3, or Amine-GlyGlu-PAB-DMEDA-INX-SM3, or other INX linker payloads wherein INX-SM3 is substituted for a payload selected from those in FIG. 118A-O or another compound of Formula I, II or III and comprises the same or different INX steroid payload via C17.

135. An antibody drug conjugate (ADC) comprising a glucocorticoid agonist compound or steroid-linker payload according to claim 133, selected from the following:
(A)
(i) Ab-Gly-Glu-PAB-DMEDA-INX-3 or Ab-GlcA-PAB-DMEDA-INX-SM-3 (alkoxyamine+ketone conjugation (C11-OH linked), or another ADC comprising a different INX-SM payload wherein INX-SM3 is substituted for a payload selected from those in FIG. 118A-O or another compound of Formula I, II or III wherein the other INX-SM payload is conjugated to the antibody via alkoxyamine+ketone conjugation and is C11-OH linked;
(ii) Ab-Gly-Glu-PAB-DMEDA-INX-3 or Ab-GlcA-PAB-DMEDA-INX-SM-3 (azide+dibenzocyclooctyne conjugation (C11-OH linked), or another ADC comprising a different INX-SM payload wherein INX-SM3 is substituted for a payload selected from those in FIG. 118A-O or another compound of Formula I, II or III wherein the INX-SM payload is conjugated to the antibody via azide+dibenzocyclooctyne conjugation and is C11-OH linked;
(iii) Ab-Gly-Glu-PAB-DMEDA-INX-3 or Ab-GlcA-PAB-DMEDA-INX-SM-3 (haloacetyl+cysteine conjugation (C11-OH linked), or another ADC comprising a different INX-SM payload wherein INX-SM3 is substituted for a payload selected from those in FIG. 118A-O or another compound of Formula I, II or III and conjugated to the antibody via azide+dibenzocyclooctyne conjugation and is C11-OH linked;
(iv) Ab-Gly-Glu-PAB-DMEDA-INX-3 or Ab-GlcA-PAB-DMEDA-INX-SM-3 (maleimide+cysteine conjugation (C11-OH linked), or another ADC comprising a different INX-SM payload wherein INX-SM3 is substituted for a payload selected from those in FIG. 118A-O or another compound of Formula I, II or III and is conjugated to the antibody via azide+dibenzocyclooctyne conjugation and is C11-OH linked;
(v) Ab-Gly-Glu-PAB-DMEDA-INX-3 or Ab-GlcA-PAB-DMEDA-INX-SM-3 (tetrazine+trans-cyclooctene conjugation (C11-OH linked), or another ADC comprising a different INX-SM payload wherein INX-SM3 is substituted for a payload selected from those in FIG. 118A-O or another compound of Formula I, II or III and is conjugated to the antibody via tetrazine+trans-cyclooctene conjugation and is C11-OH linked;
(vi) Ab-Gly-Glu-PAB-DMEDA-INX-3 or Ab-GlcA-PAB-DMEDA-INX-SM-3 (Alkoxyamine+Ketone conjugation (C11-OH linked)), or another ADC comprising a different INX-SM payload wherein INX-SM3 is substituted for a payload selected from those in FIG. 118A-O or another compound of Formula I, II or III is conjugated to the antibody via alkoxyamine+ketone conjugation and is C11-OH linked;
(vii) Ab-Gly-Glu-PAB-DMEDA-INX-3 or Ab-GlcA-PAB-DMEDA-INX-SM-3 (Azide+Dibenzocyclooctyne conjugation (C17 linked)), or another ADC comprising a different INX-SM payload wherein INX-SM3 is substituted for a payload selected from those in FIG. 118A-O or another compound of Formula I, II or III and is conjugated to the antibody via azide+dibenzocyclooctyne ketone conjugation and is C17 linked;
(viii) Ab-Gly-Glu-PAB-DMEDA-INX-3 or Ab-GlcA-PAB-DMEDA-INX-SM-3 (haloacetyl+cysteine conjugation (C17 linked)), or another ADC comprising a different INX-SM payload wherein INX-SM3 is substituted for a payload selected from those in FIG. 118A-O or another compound of Formula I, II or III and is conjugated to the antibody via azide+dibenzocyclooctyne conjugation and is C17 linked;
(ix) Ab-Gly-Glu-PAB-DMEDA-INX-3 or Ab-GlcA-PAB-DMEDA-INX-SM-3 (Tetrazine+Trans-cyclooctene conjugation (C17 linked)), or another ADC comprising a different INX-SM payload wherein the INX-SM linker payload and is conjugated to the antibody via tetrazine+trans-cyclooctene conjugation and is C17 linked;
(x) Ab-Gly-Glu-PAB-DMEDA-INX-3 or Ab-GlcA-PAB-DMEDA-INX-SM-3 (amine+gutamine conjugation using trans glutaminase (C17 linked)), or another ADC comprising a different INX-SM payload wherein the INX-SM linker payload and is conjugated to the antibody via amine+glutamine conjugation using trans glutaminase and is C17 linked;
(xi) INX-SM-3-PAB-GlcA-Ab or INX-SM-3-PAB-Glu-Gly-Ab (alkoxyamine and ketone conjugation) (N-linked payload) or another ADC comprising a different INX-SM payload wherein INX-SM3 is substituted for a payload selected from those in FIG. 118A-O or another compound of Formula I, II or III payload and is conjugated to the antibody via alkoxyamine and ketone conjugation and is N linked;
(xii) INX-SM-3-PAB-GlcA-Ab or INX-SM-3-PAB-Glu-Gly-Ab (haloacetyl Conjugation) (N-linked payload) or another ADC comprising a different INX-SM payload wherein INX-SM3 is substituted for a payload selected from those in FIG. 118A-O or another compound of Formula I, II or III payload and is conjugated to the antibody via haloacetyl conjugation and is N linked;
(xiii) INX-SM-3-PAB-GlcA-Ab or INX-SM-3-PAB-Glu-Gly-Ab (azide+dibenzocyclooctyne conjugation conjugation) (N-linked payload) or another ADC comprising a different INX-SM payload wherein INX-SM3 is substituted for a payload selected from those in FIG. 118A-O or another compound of Formula I, II or III payload and is conjugated to the antibody via azide+dibenzocyclooctyne conjugation and is N linked;
(xiv) INX-SM-3-GlcA-Ab or INX-SM-3-Glu-Gly-Ab (N-hydroxysuccinimide conjugation) (N-linked payload) or another ADC comprising a different INX-SM payload wherein INX-SM3 is substituted for a payload selected from those in FIG. 118A-O or another compound of Formula I, II or III payload and is conjugated to the antibody via N-hydroxysuccinimide conjugation and is N linked;
(xv) INX-SM-3-PAB-GlcA-Ab or INX-SM-3-PAB-Glu-Gly-Ab (Azide+Dibenzocyclooctyne conjugation) (N-linked payload) or another ADC comprising a different INX-SM payload wherein INX-SM3 is substituted for a payload selected from those in FIG. 118A-O or another compound of Formula I, II or III payload and is conjugated to the antibody via azide+dibenzocyclooctyne conjugation and is N linked;
(xvi) INX-SM-3-PAB-GlcA-Ab or INX-SM-3-PAB-Glu-Gly-Ab (N-hydroxysuccinimide Conjugation) (N-linked payload) or another ADC comprising a different INX-SM payload wherein INX-SM3 is substituted for a payload selected from those in FIG. 118A-O or another compound of Formula I, II or III payload and is conjugated to the antibody via N-hydroxysuccinimide conjugation and is N linked;
(xvii) INX-SM-3-Glu-Gly-Ab or INX-SM-3-PAB-Glu-Gly-Ab (Maleimide Conjugation) (N-linked payload) or another ADC comprising a different INX-SM payload wherein INX-SM3 is substituted for a payload selected from those in FIG. 118A-O or another compound of Formula I, II or III payload and is conjugated to the antibody via maleimide conjugation and is N linked;
(xviii) INX-SM-3-Glu-Gly-Ab or INX-SM-3-PAB-Glu-Gly-Ab or INX-SM-3-PAB-GlcA-Ab (trans-cyclooctene+tetrazine conjugation) (N-linked payload) or another ADC comprising a different INX-SM payload wherein INX-SM3 is substituted for a payload selected from those in FIG. 118A-O or another compound of Formula I, II or III payload and is conjugated to the antibody via trans-cyclooctene+tetrazine Conjugation and is N linked;
(xix) INX-SM-3-Glu-Gly-Ab or INX-SM-3-PAB-Glu-Gly-Ab or INX-SM-3-PAB-GlcA-Ab (amine conjugation) (N-linked payload) or another ADC comprising a different INX-SM payload wherein INX-SM3 is substituted for a payload selected from those in FIG. 118A-O or another compound of Formula I, II or III payload and is conjugated to the antibody via trans-cyclooctene+tetrazine conjugation and is N linked; or
(B)
an ADC selected from the following: 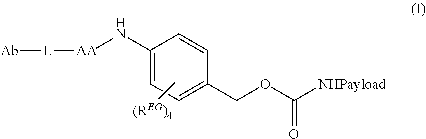
wherein,
Ab=Antibody, preferably an antibody that binds to human immune cells, preferably an anti-VISTA antibody that binds to human VISTA immune cells at physiologic pH;
L=Linker;
AA=Single, double, or triple amino acid sequence; 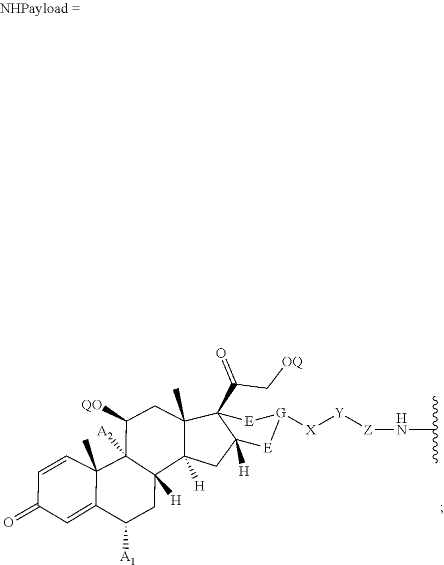
R^{EG} is independently selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—; 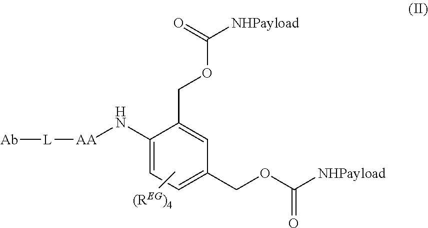
Ab=Antibody;
L=Linker;
AA=Single, double, or triple amino acid sequence; 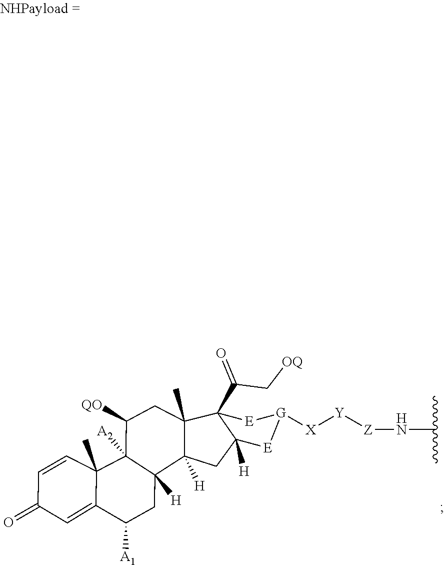
R^{EG} is independently selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—; 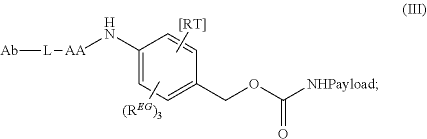
Ab=Antibody;
L=Linker;
AA=Single, double, or triple amino acid sequence or not present; 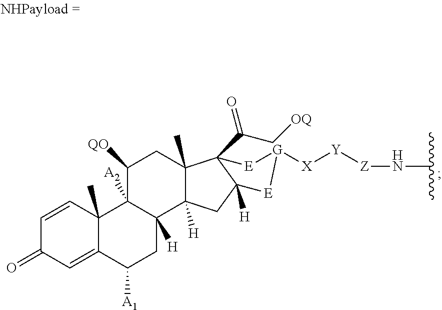
R^{EG} is independently selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—; 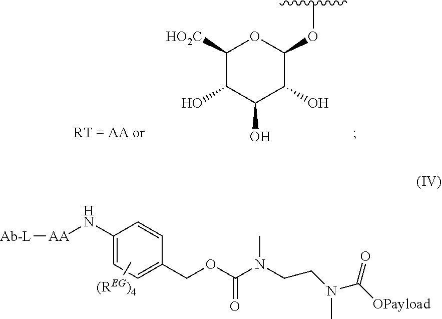
Ab=Antibody, optionally an anti-human VISTA antibody;
L=Linker;
AA=Single, double, or triple amino acid sequence; 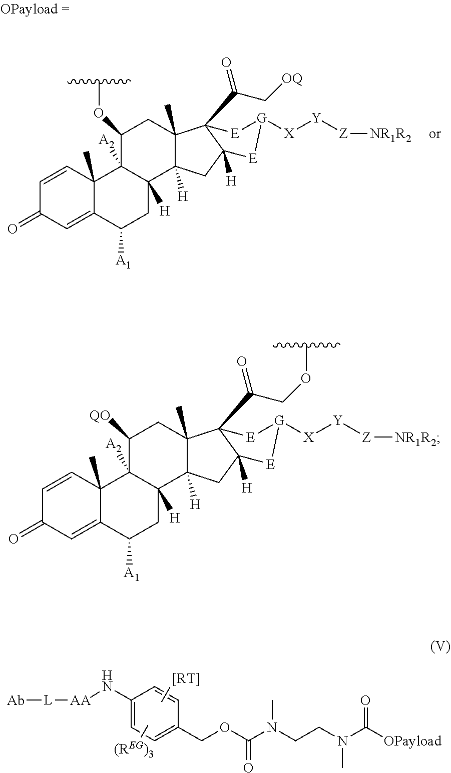
Ab=Antibody;
L=Linker;
AA=Single, double, or triple amino acid sequence or not present; 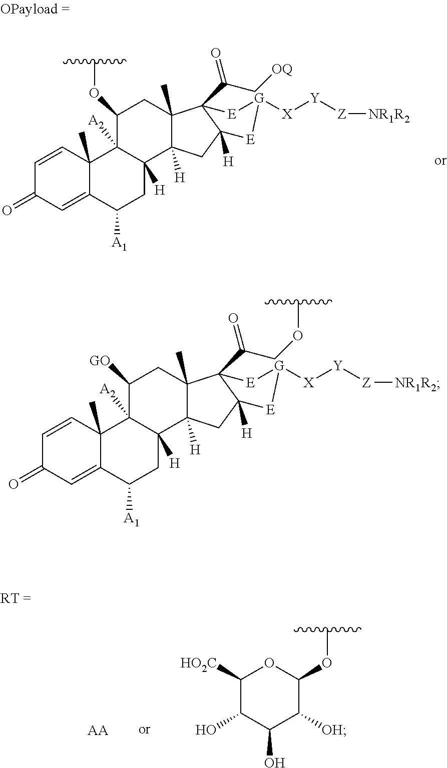
optionally wherein the linker comprises a cleavable or non-cleavable peptide or immolative linker; or which comprises a linker which is selected from PAB and/or an amino acid or a peptide, optionally 1-12 amino acids, further optionally dipeptide, a tripeptide, a quatrapeptide, a pentapeptide and further optionally Gly, Asn, Asp, Gln, Leu, Lys, Ala, Phe, Cit, Val, Val-Cit, Val-Ala, Val-Gly, Val-Gln, Ala-Val, Cit-Cit, Lys-Val-Cit, Asp-Val-Ala, Ala-Ala-Asn, Asp-Val-Ala, Ala-Val-Cit, Ala-Asn-Val, betaAla-LeuAla-Leu, Lys-Val-Ala, Val-Leu-Lys, Asp-Val-Cit, Val-Ala-Val, and Ala-Ala-Asn; or
(C)
said ADC is selected from the following structures: 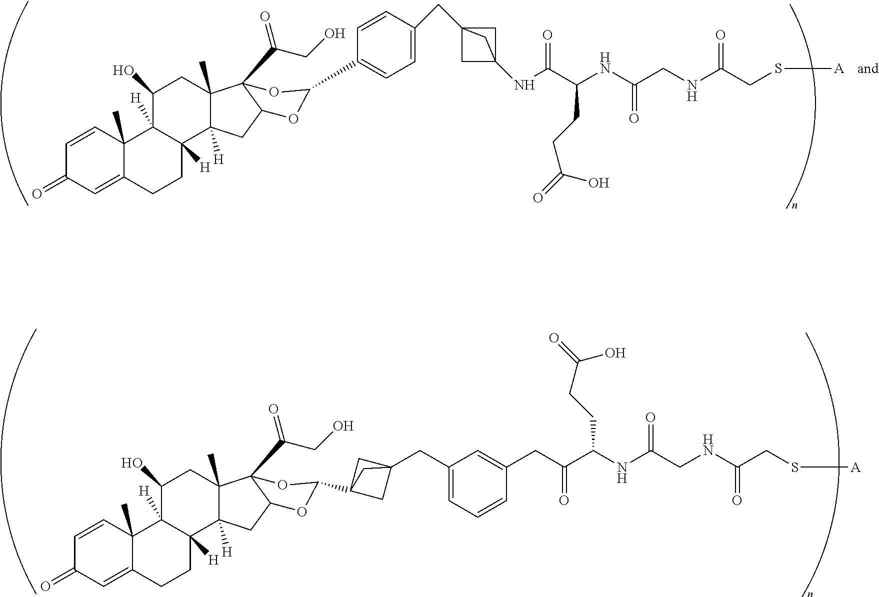
where n=2-12, 2-10, 2=8, 2-6, 2-4 and A is an antibody or antigen binding fragment thereof, preferably an antibody or antibody fragment which binds to an antigen expressed on an immune cell, preferably a human immune cell, more preferably an anti-human VISTA antibody; or
(D)
said ADC comprises an antibody or antigen binding fragment thereof, preferably one which binds to an antigen expressed by an immune cell, preferably a human immune cell, which antibody or antigen binding fragment thereof, is attached to at least one glucocorticoid agonist compound or steroid-linker according to any one of the previous claims or one shown in FIG. 118A-O; or
(E)
said ADC of (D) is selected from: 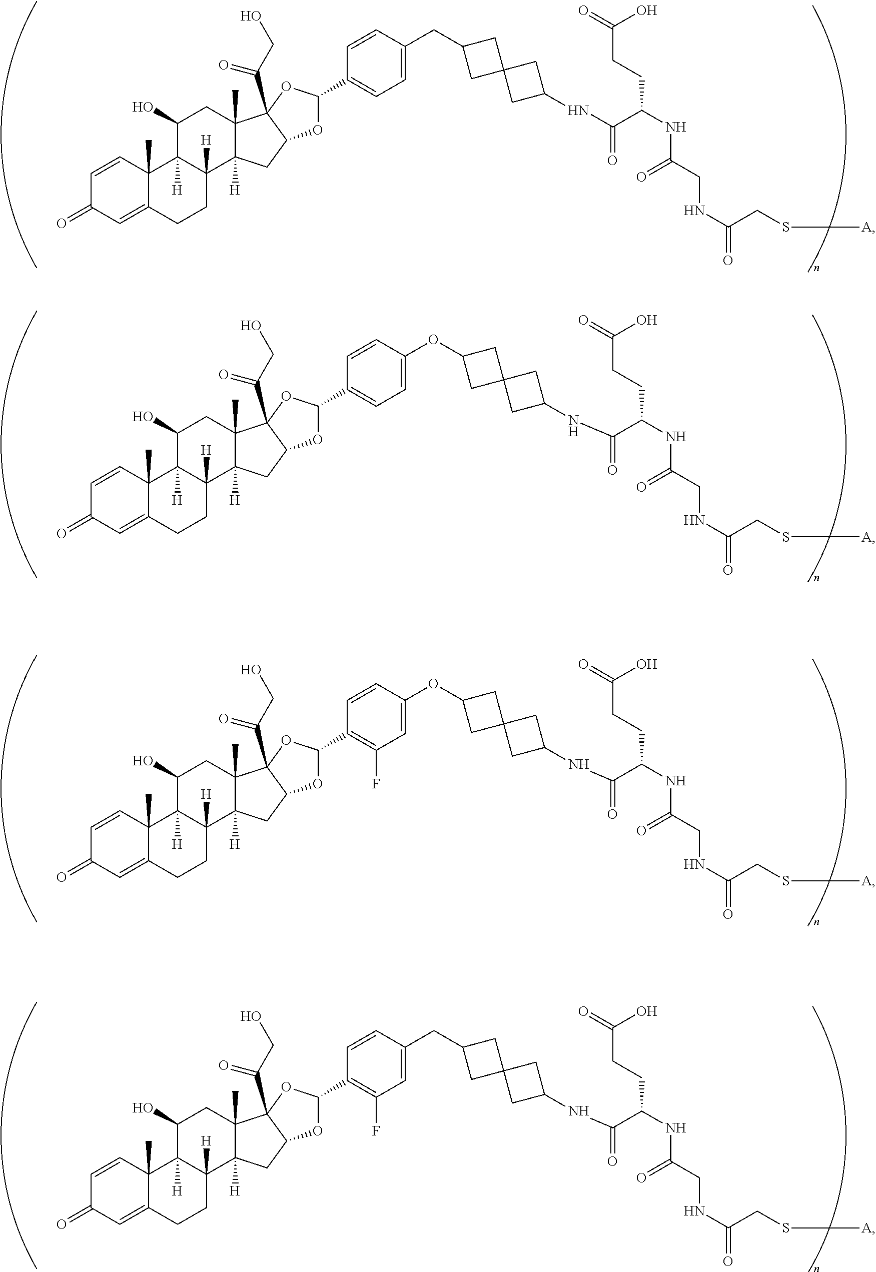 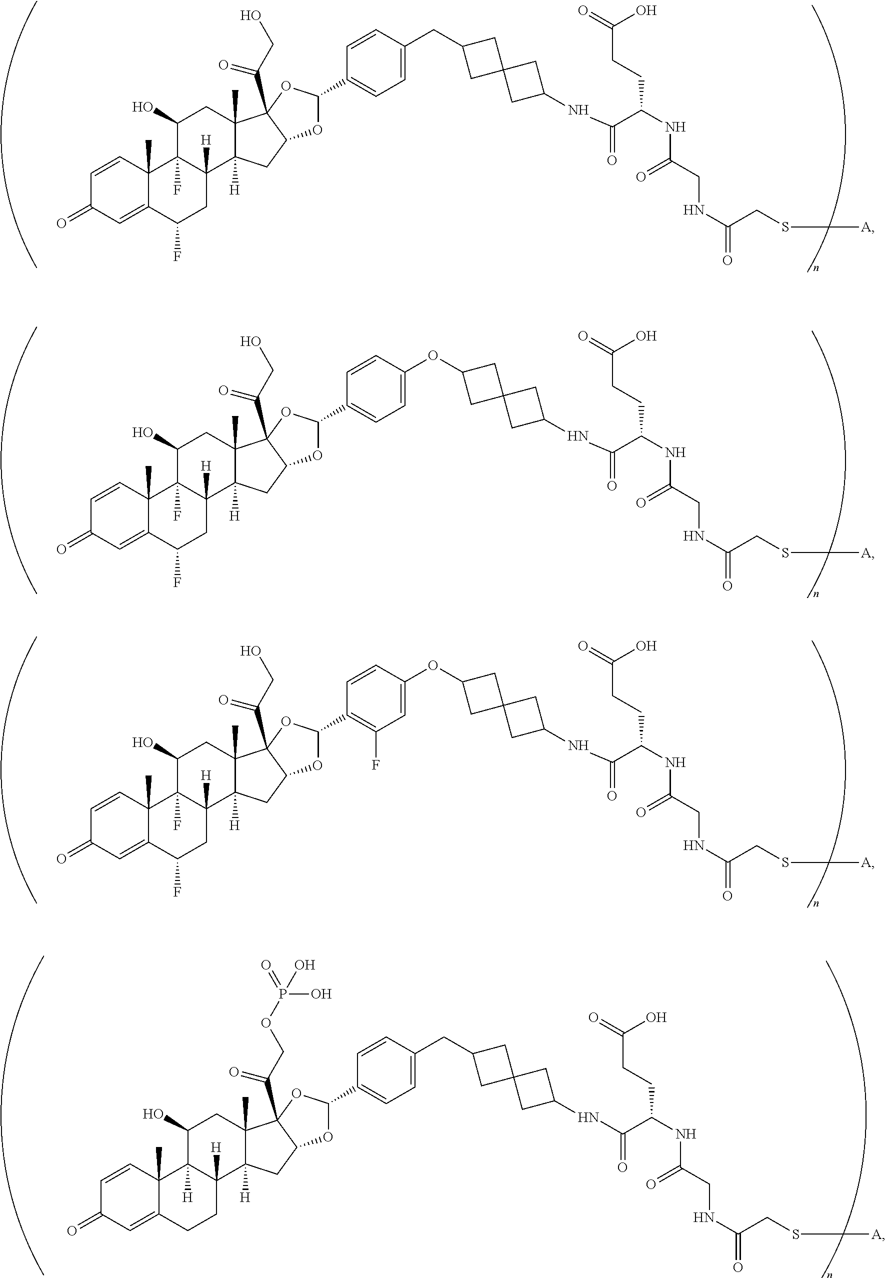 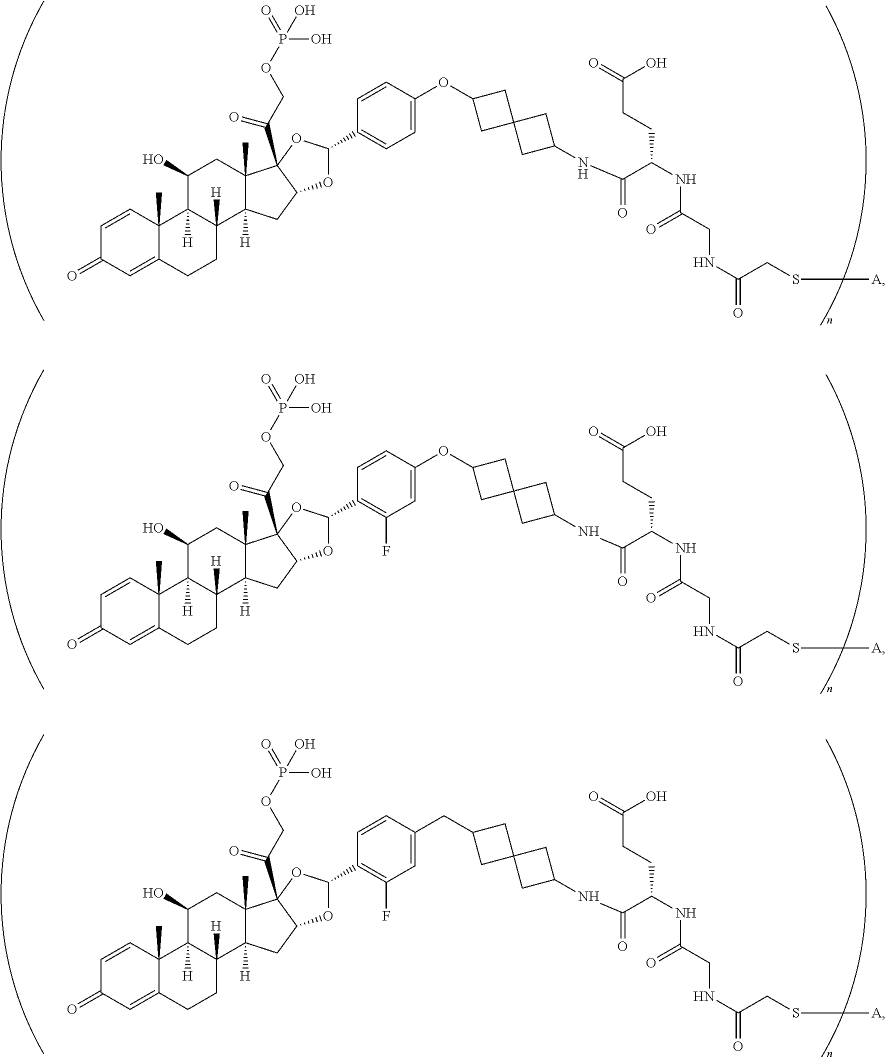 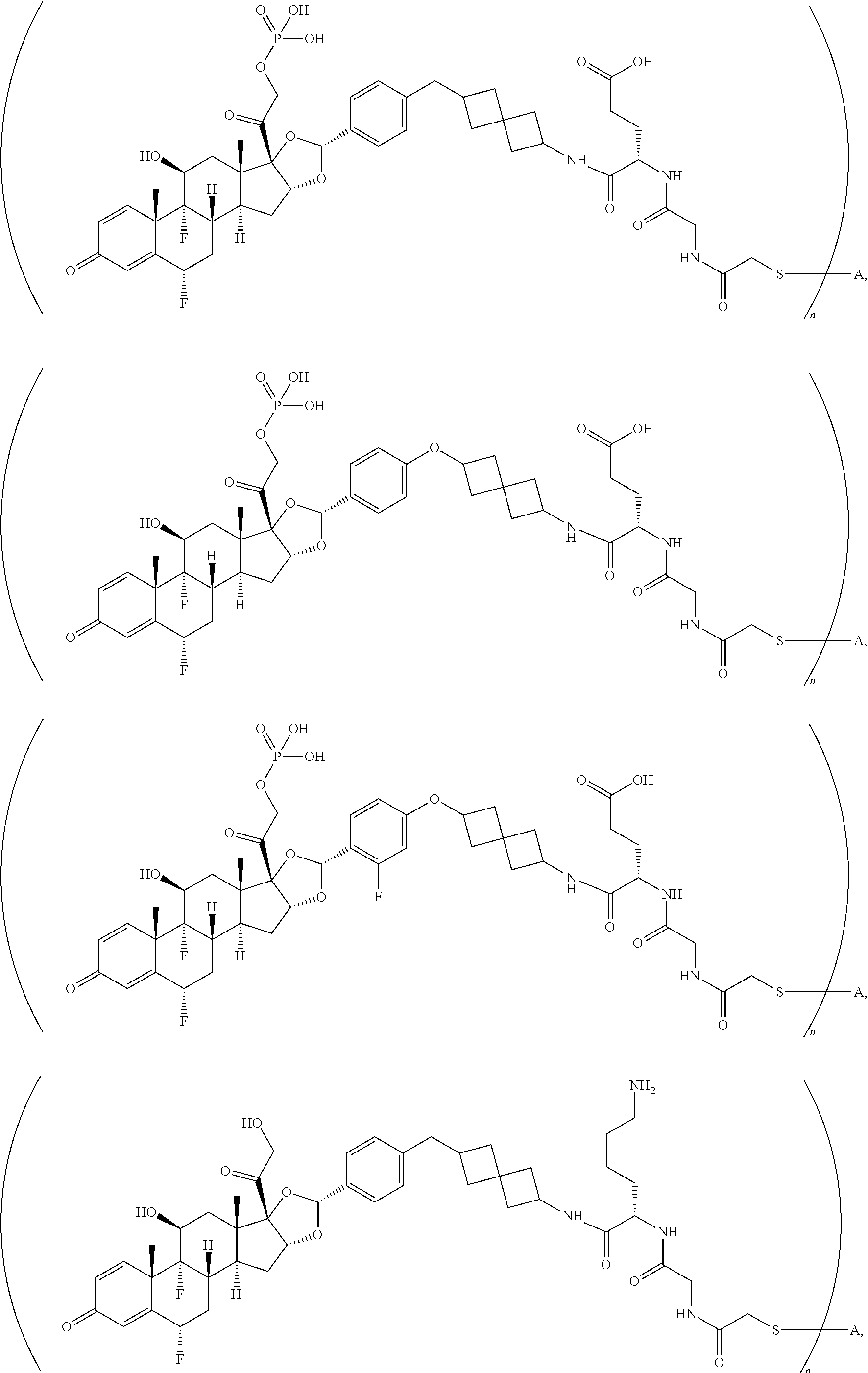 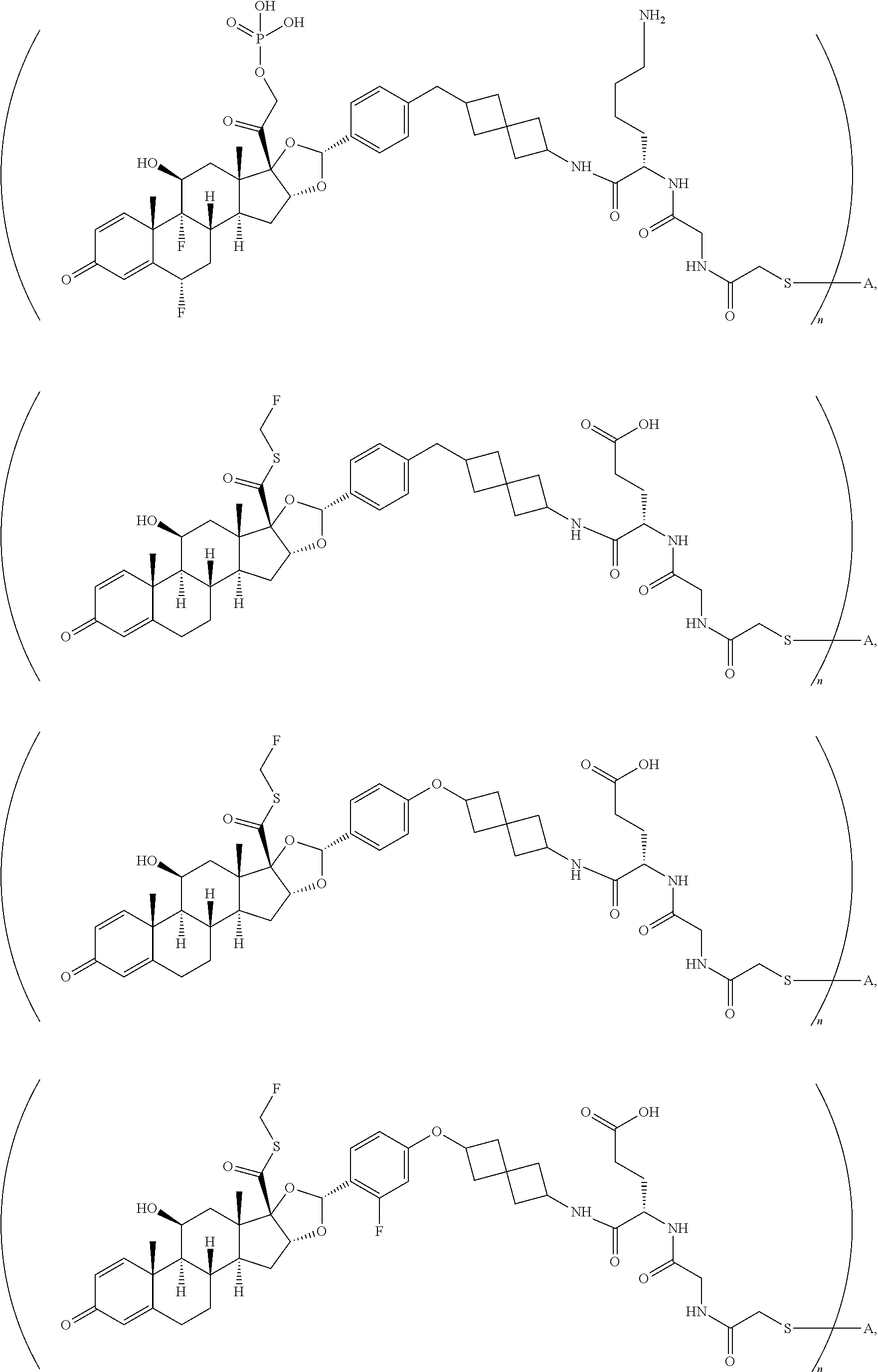 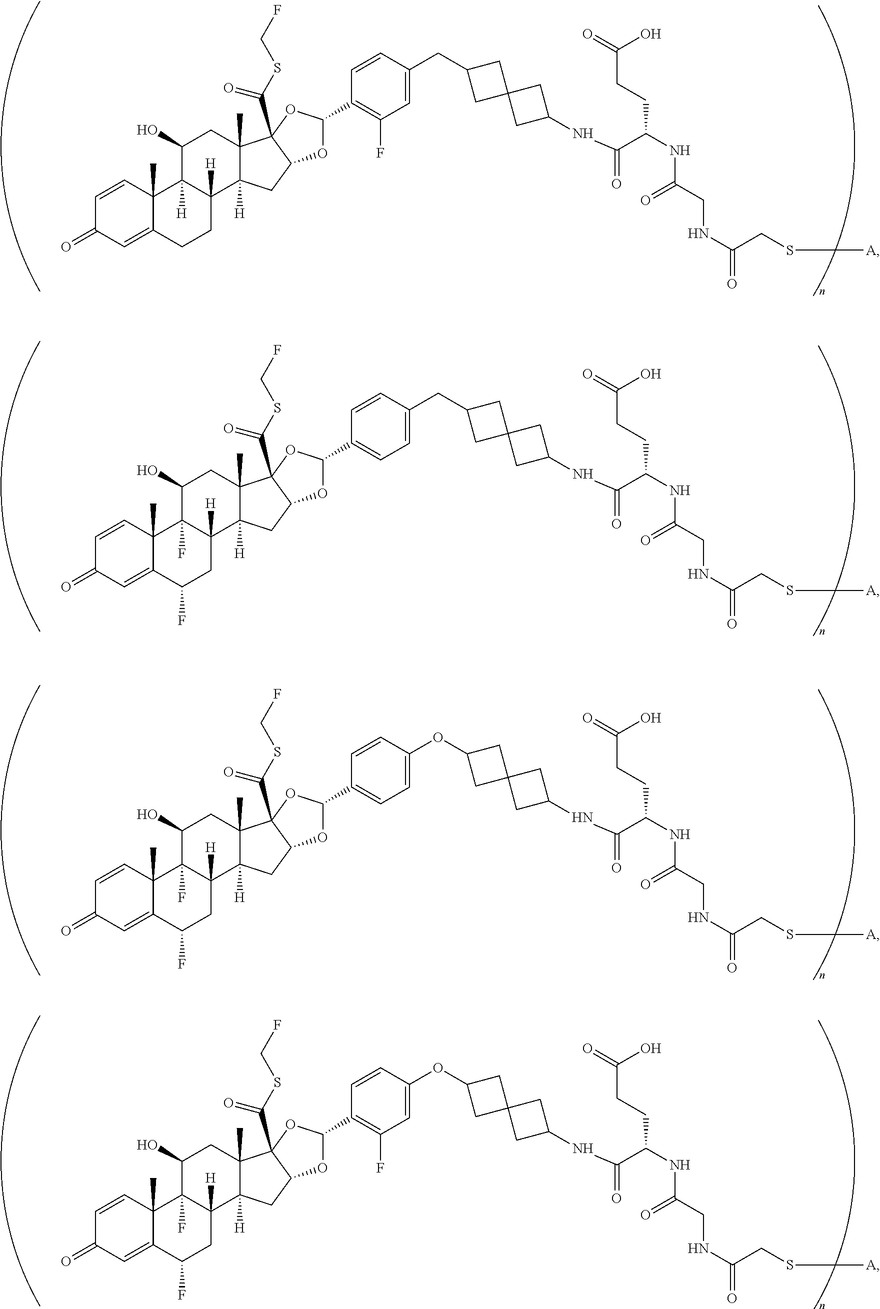 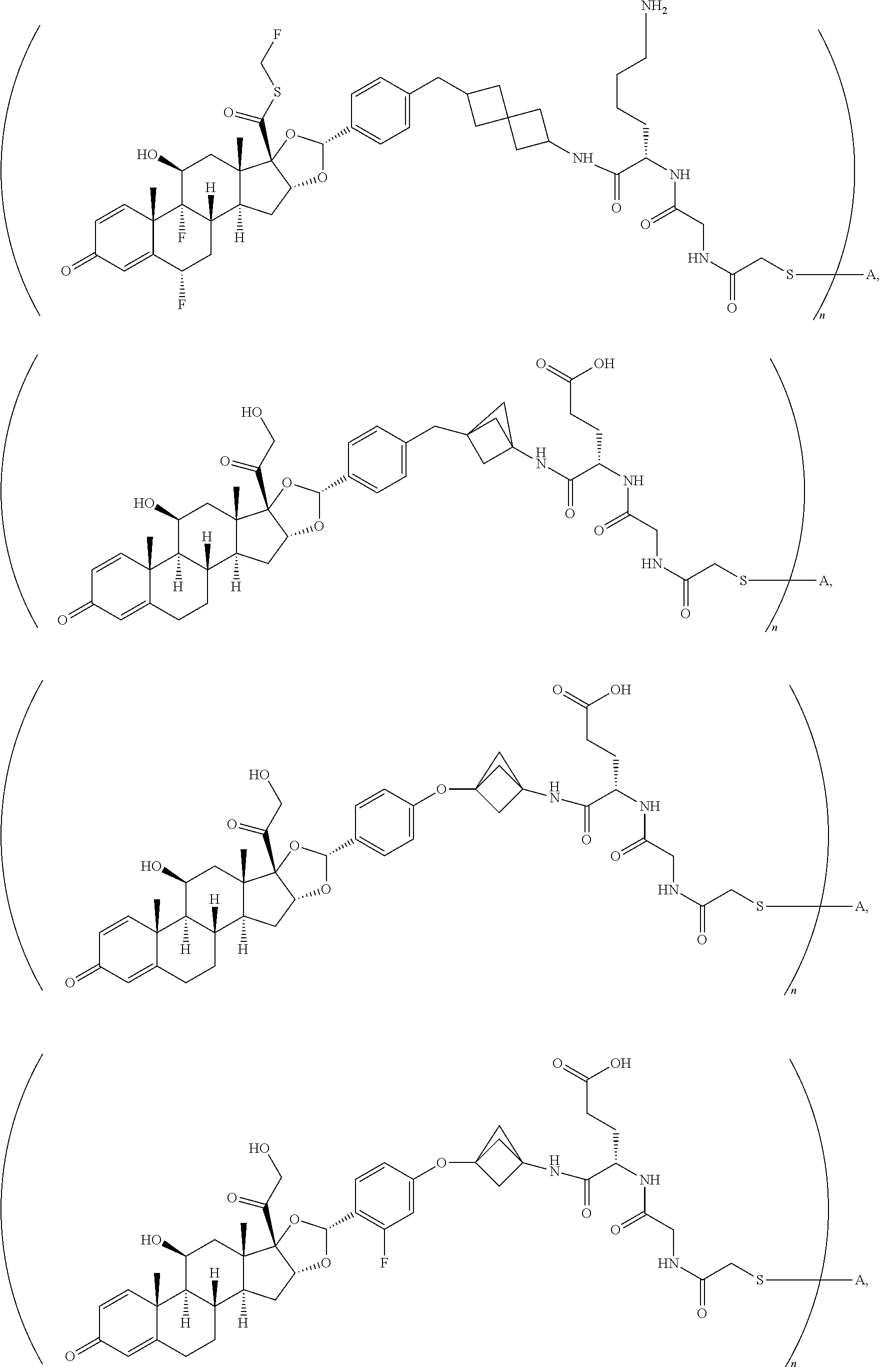 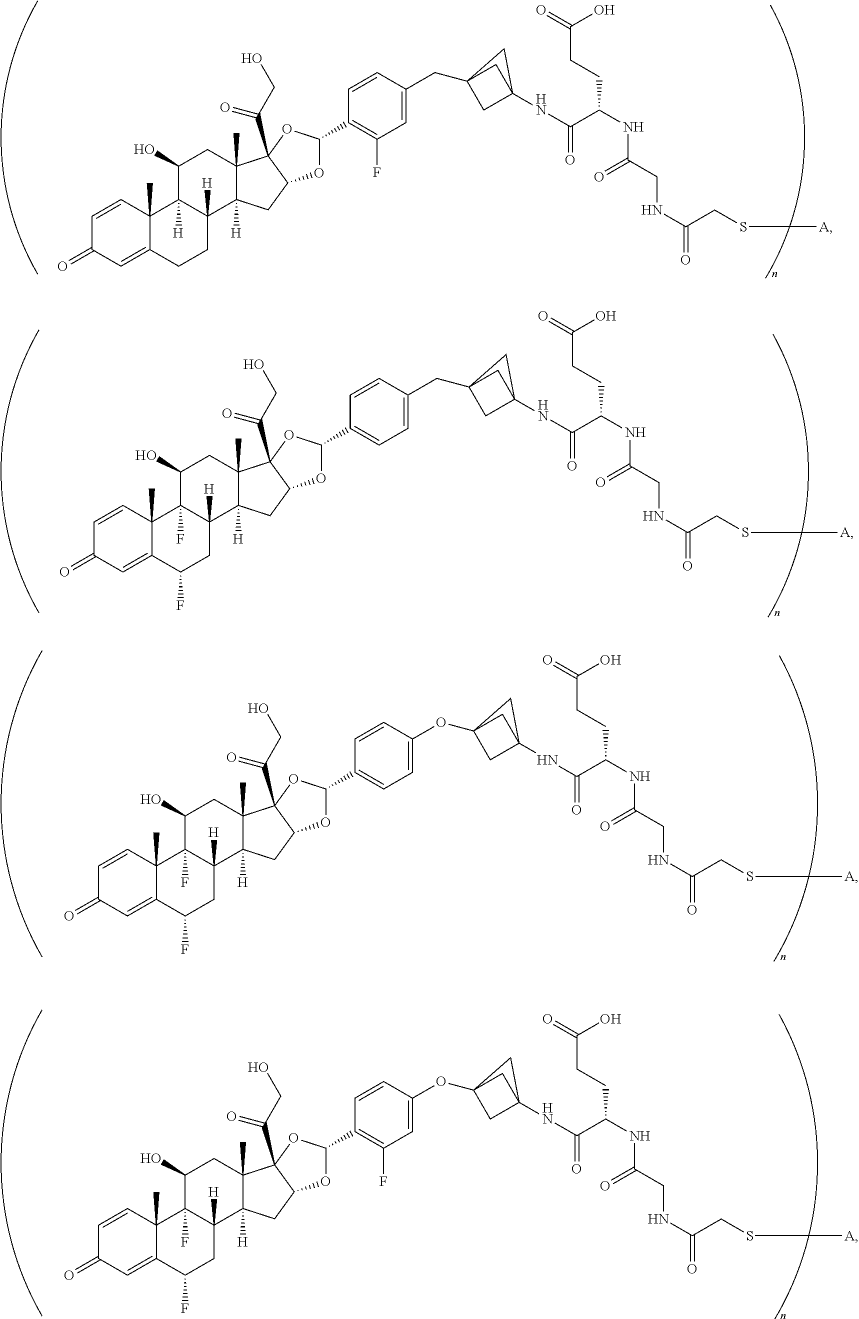 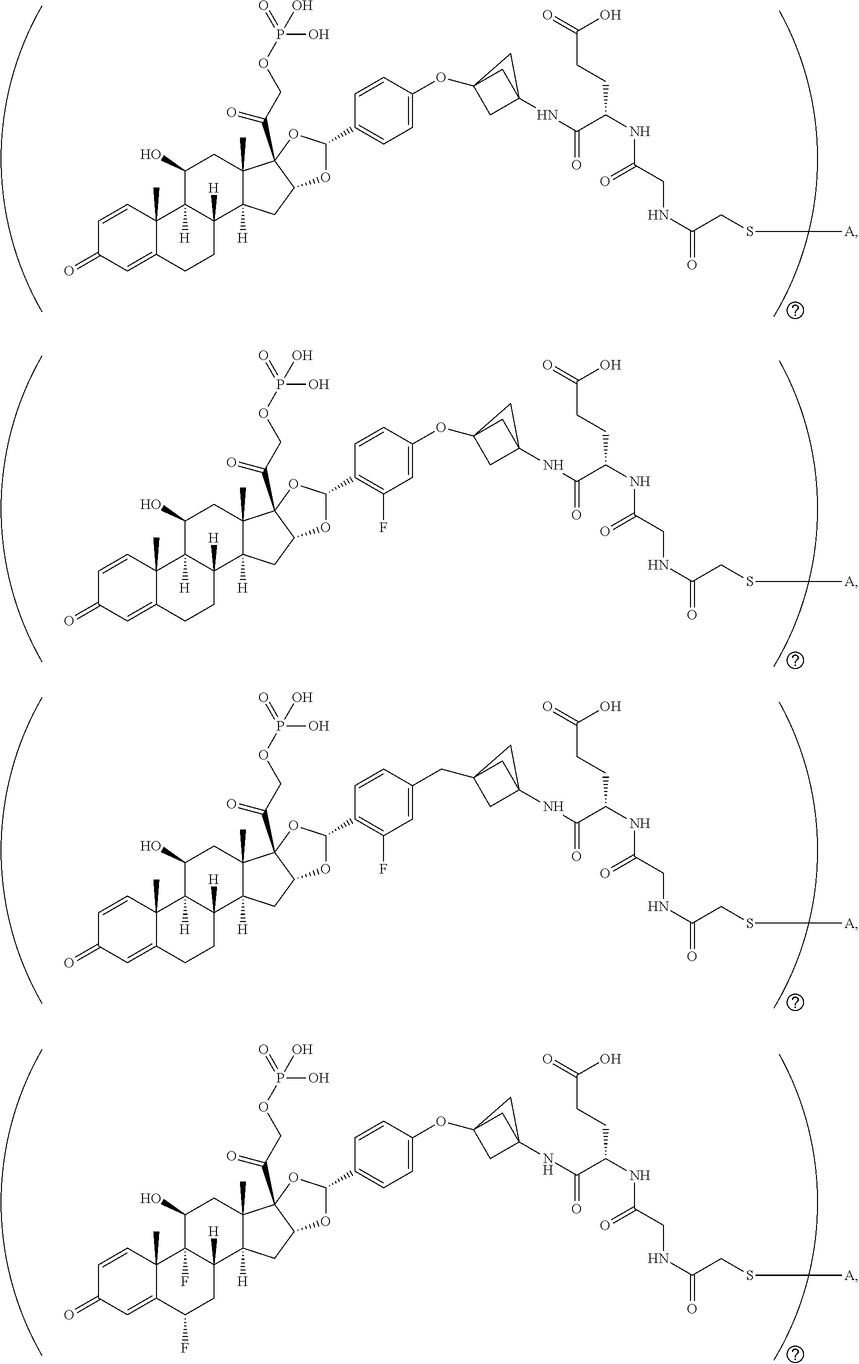 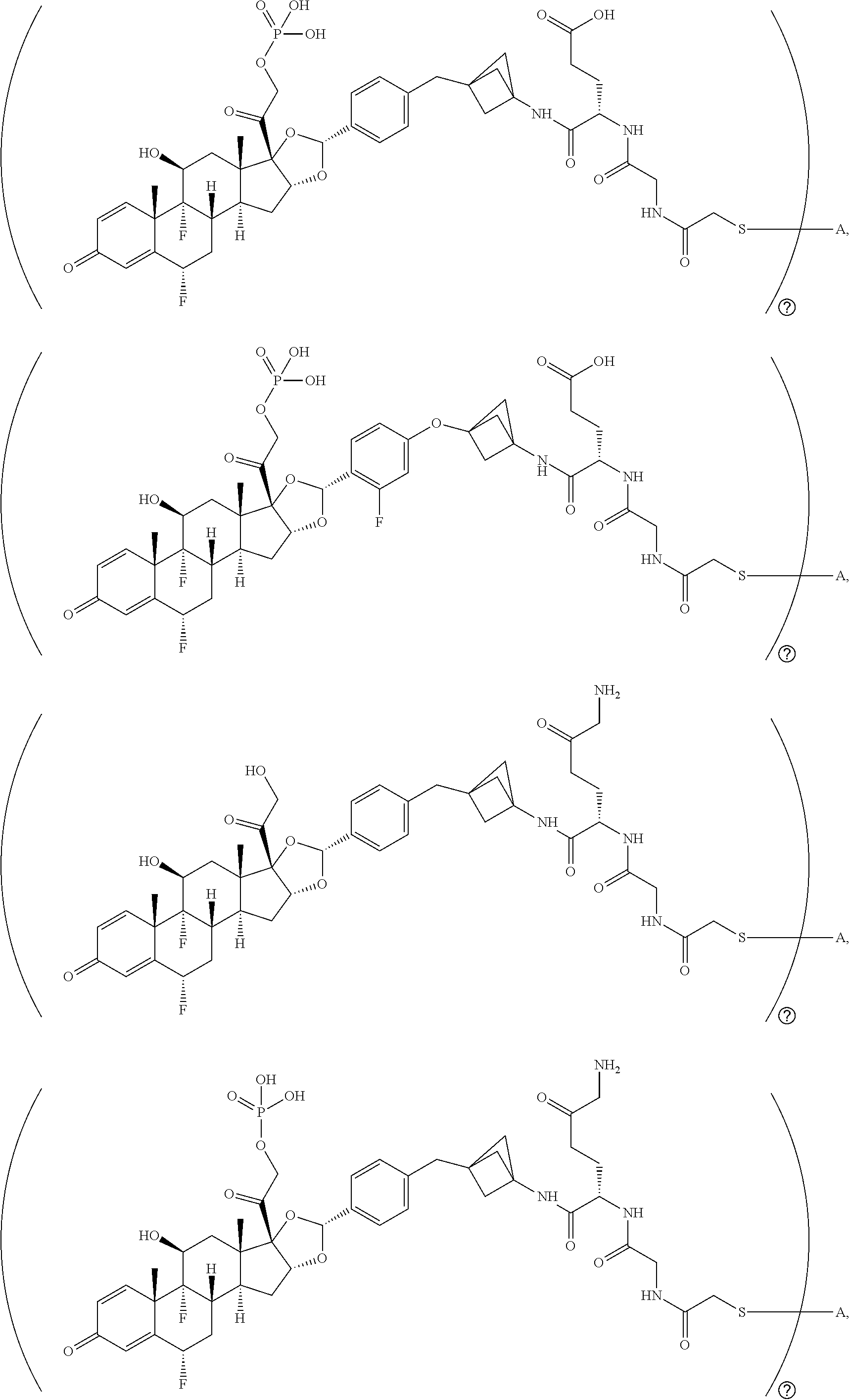 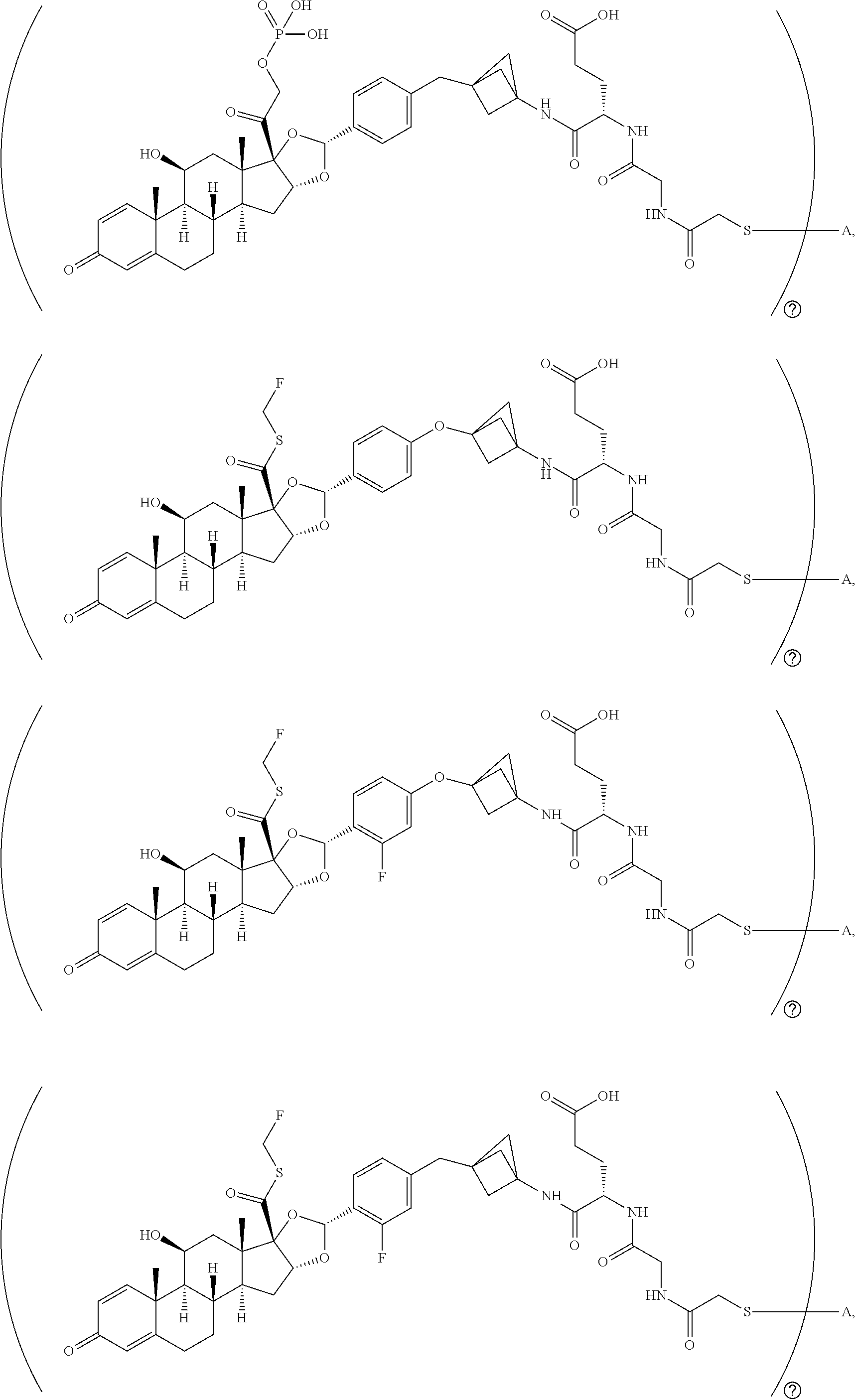 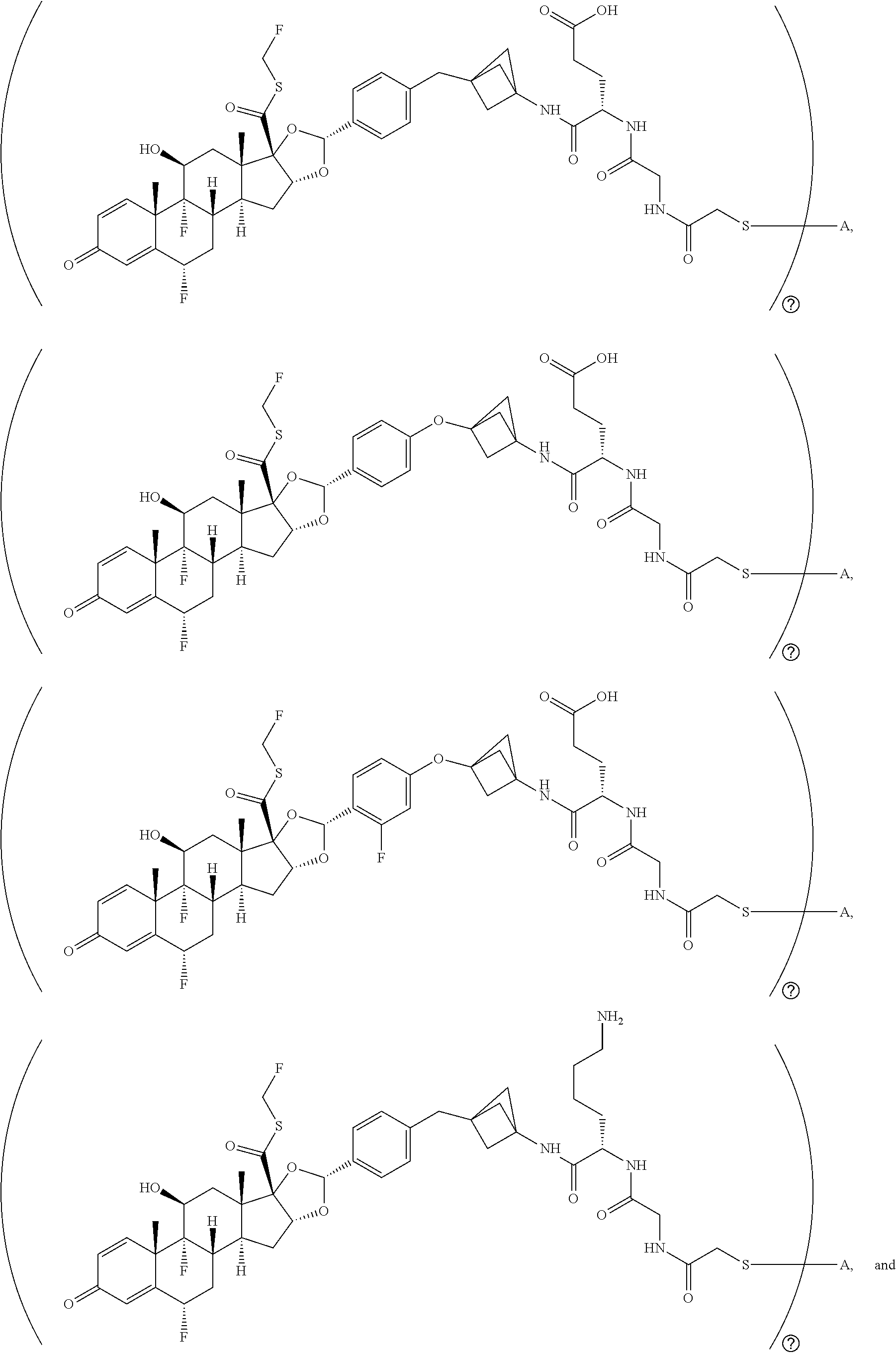 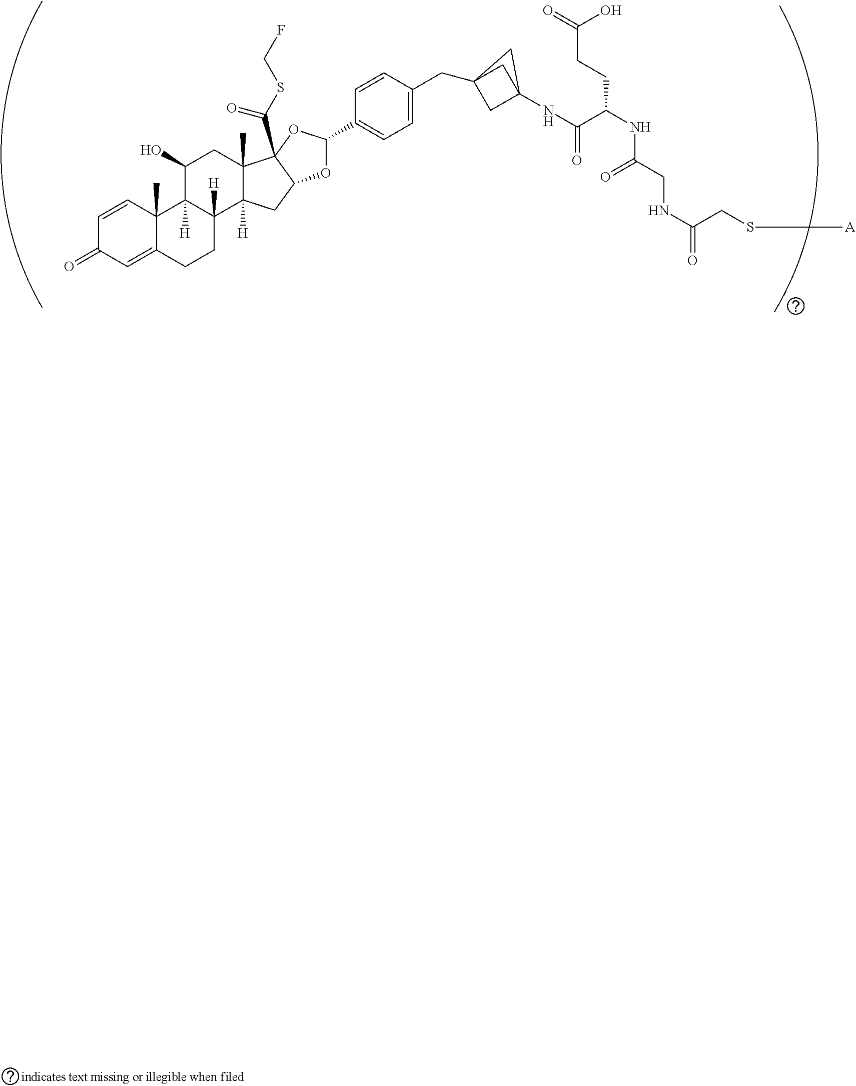
preferably where n=2-12, 2-10, 2-8, 2-6, or 2-4 and A is an antibody which binds to an antigen expressed by an immune cell, preferably a human immune cell and more preferably an anti-human VISTA antibody.

136. A steroid-linker payload comprising a glucocorticoid agonist according to claim 133, wherein
(i) the linker is selected from any of those disclosed herein or exemplified in the examples or the compounds shown in FIG. 118A-O and FIG. 11;
(ii) it comprises at least one cleavable or non-cleavable linker selected from PAB and/or or an amino acid or a peptide, optionally 1-12 amino acids, further optionally dipeptide, a tripeptide, a quatrapeptide, a pentapeptide and further optionally Gly, Asn, Asp, Gln, Leu, Lys, Ala, Phe, Cit, Val, Val-Cit, Val-Ala, Val-Gly, Val-Gln, Ala-Val, Cit-Cit, Lys-Val-Cit, Asp-Val-Ala, Ala-Ala-Asn, Asp-Val-Ala, Ala-Val-Cit, Ala-Asn-Val, betaAla-Leu-Ala-Leu, Lys-Val-Ala, Val-Leu-Lys, Asp-Val-Cit, Val-Ala-Val, and Ala-Ala-Asn; or optionally at least one of GlcA, PAB, and Glu-Gly; or
(iii) it comprises at least one cleavable linker, and/or an immolative linker, which is directly or indirectly attached to the glucocorticoid agonist steroid compound.

137. An antibody drug conjugate (ADC) according to claim 135, which
(i) comprises an antibody or antigen binding fragment thereof, preferably one which binds to an antigen expressed by an immune cell, preferably an antigen expressed on a human immune cell, which antibody or antigen binding fragment thereof, is attached to at least one glucocorticoid agonist or steroid-linker compound according to any one of the previous claims;
(ii) is selected from: 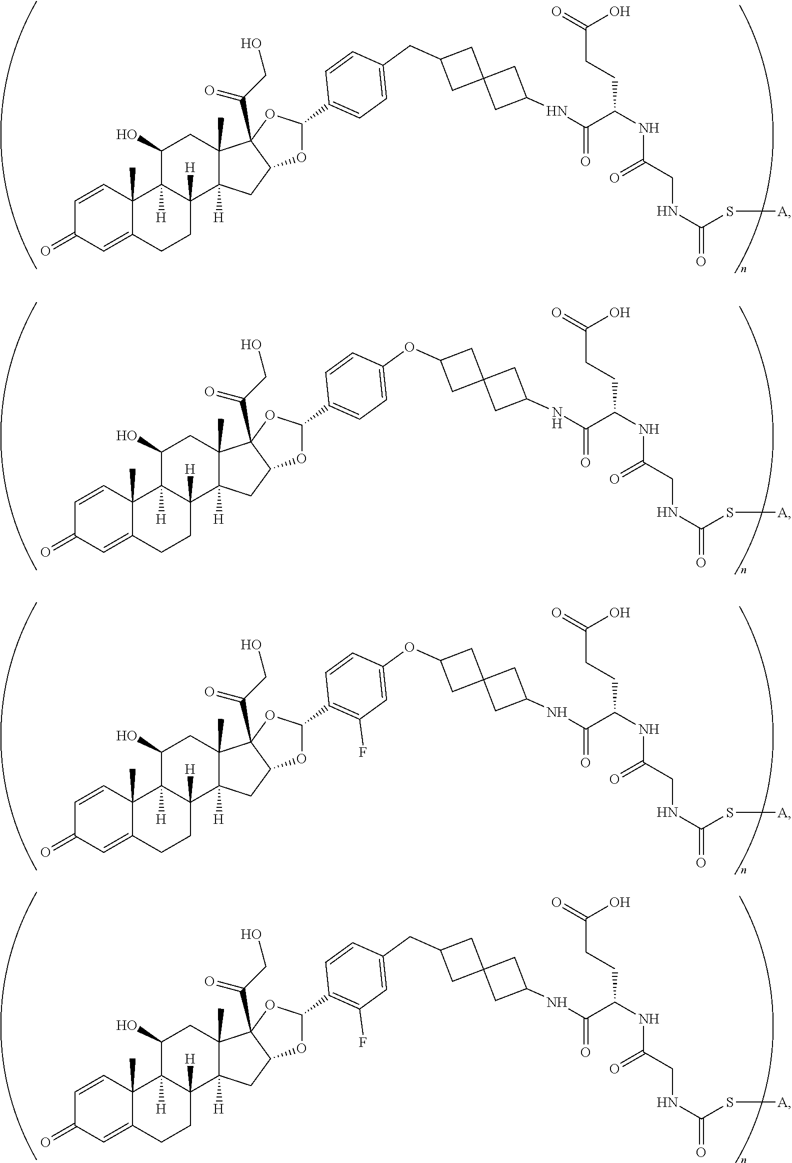 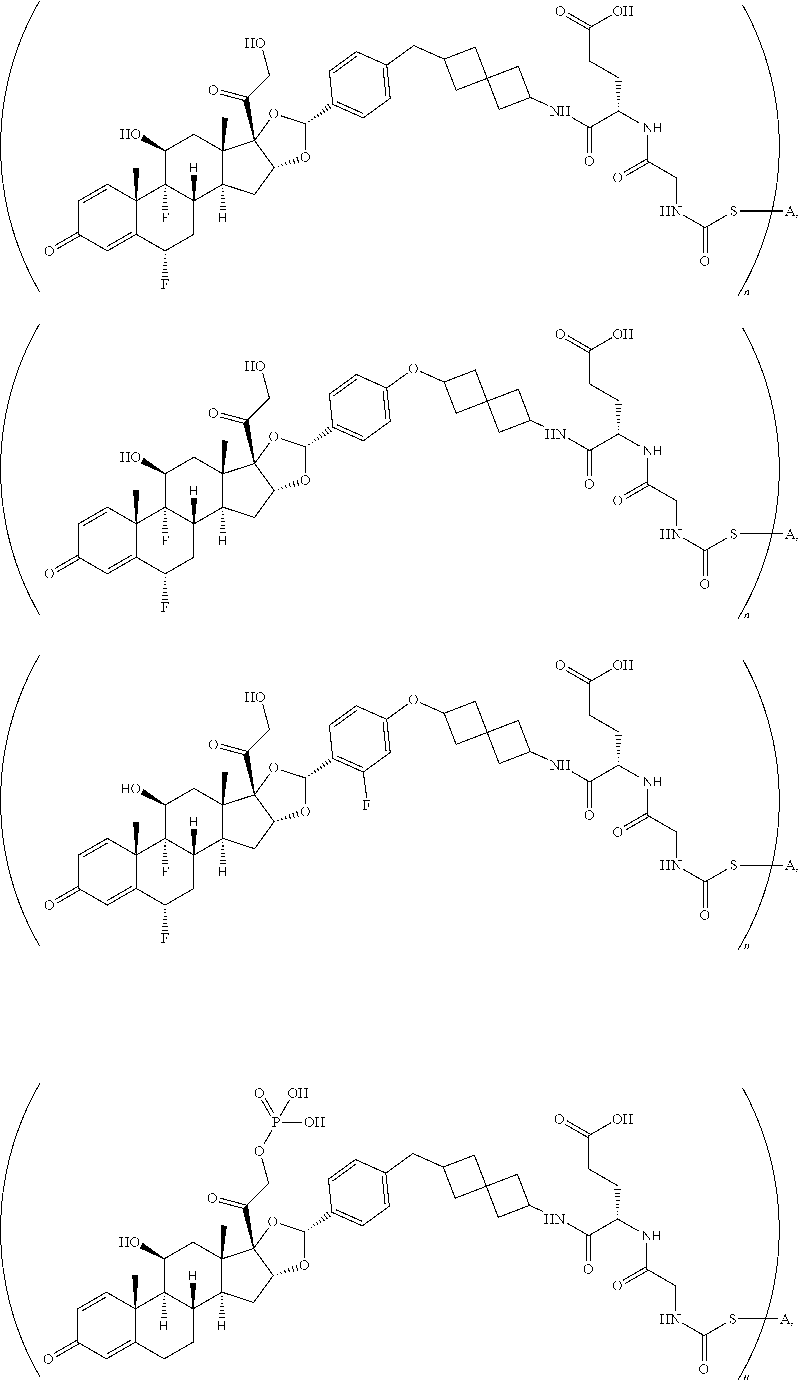 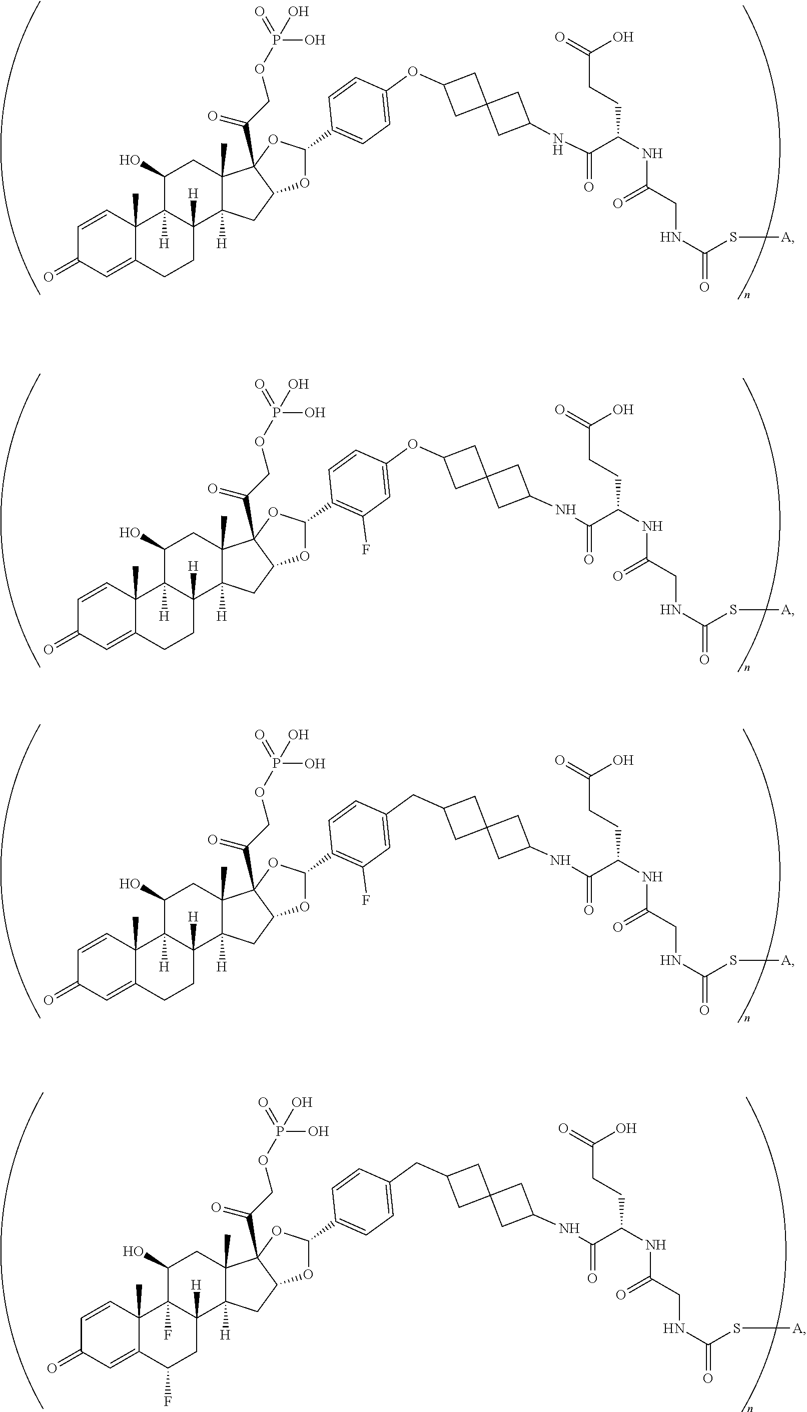 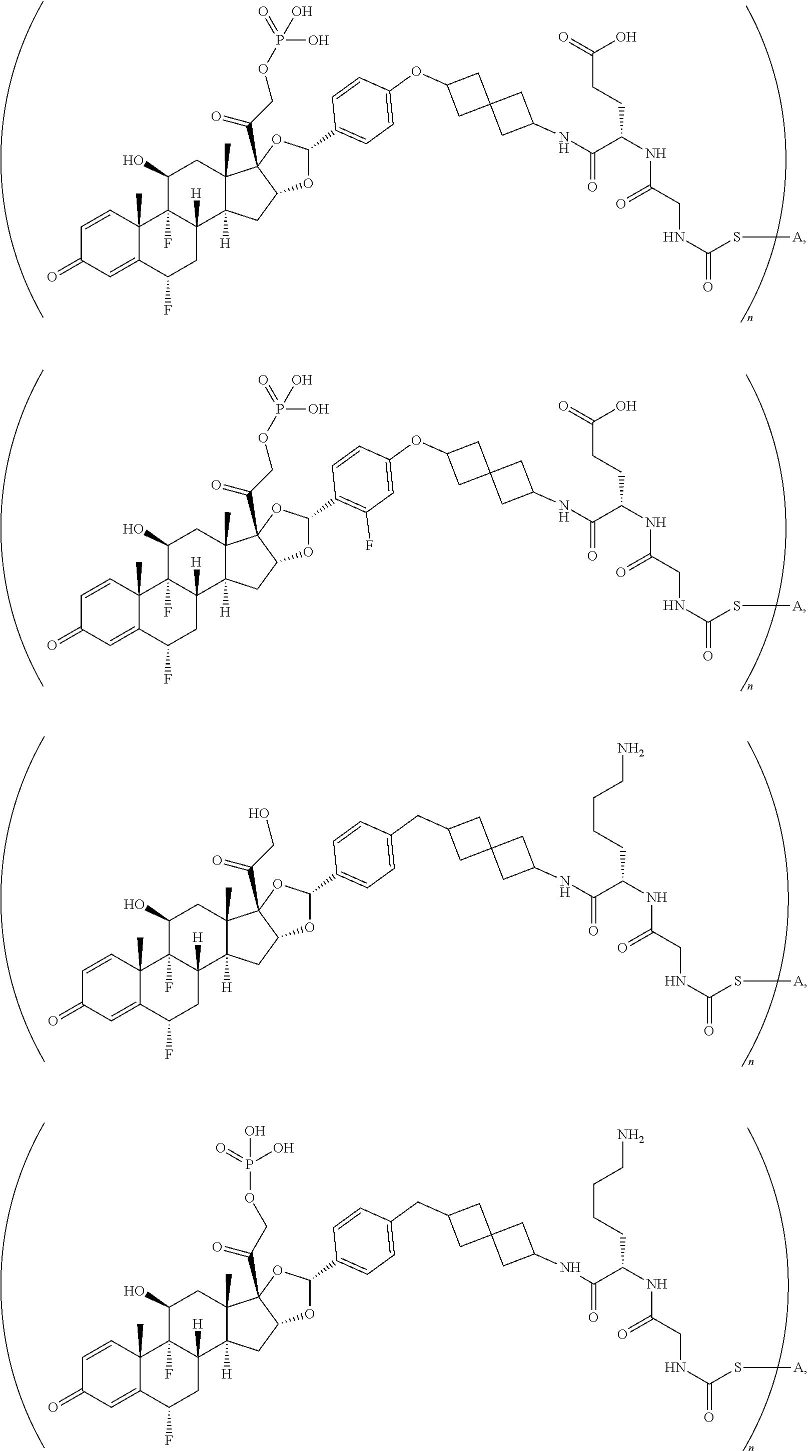 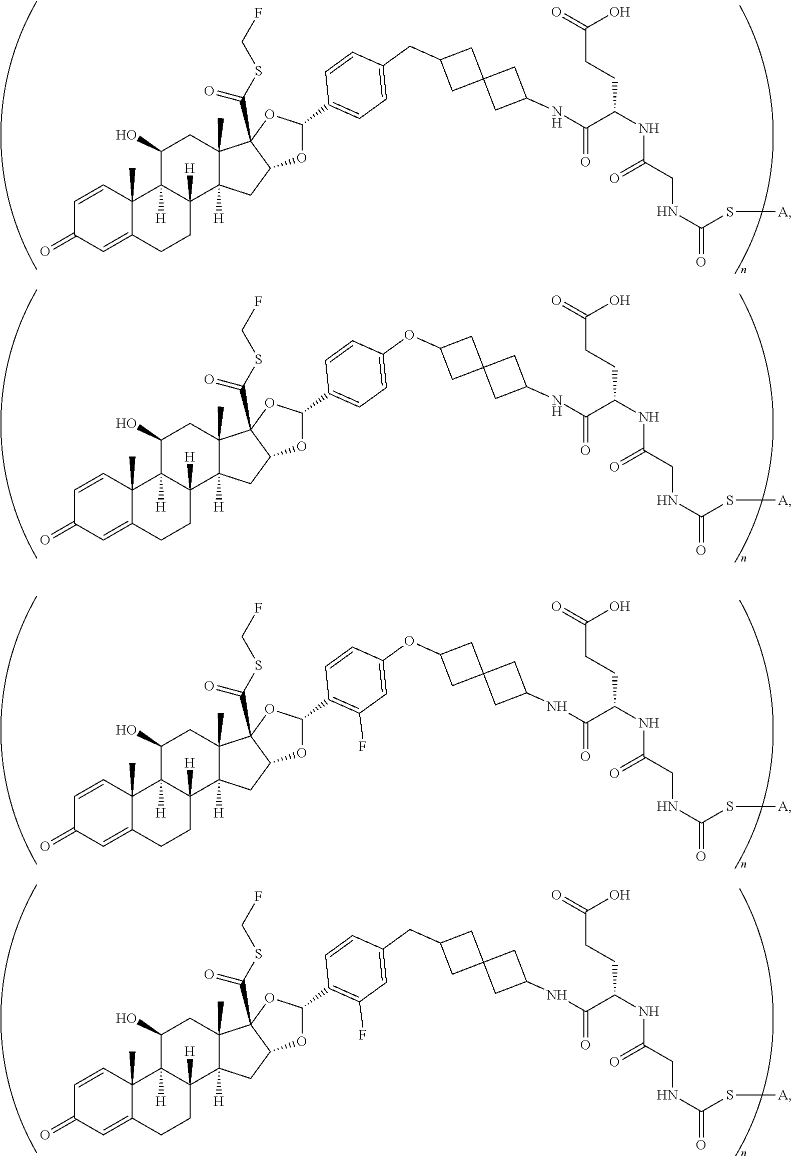 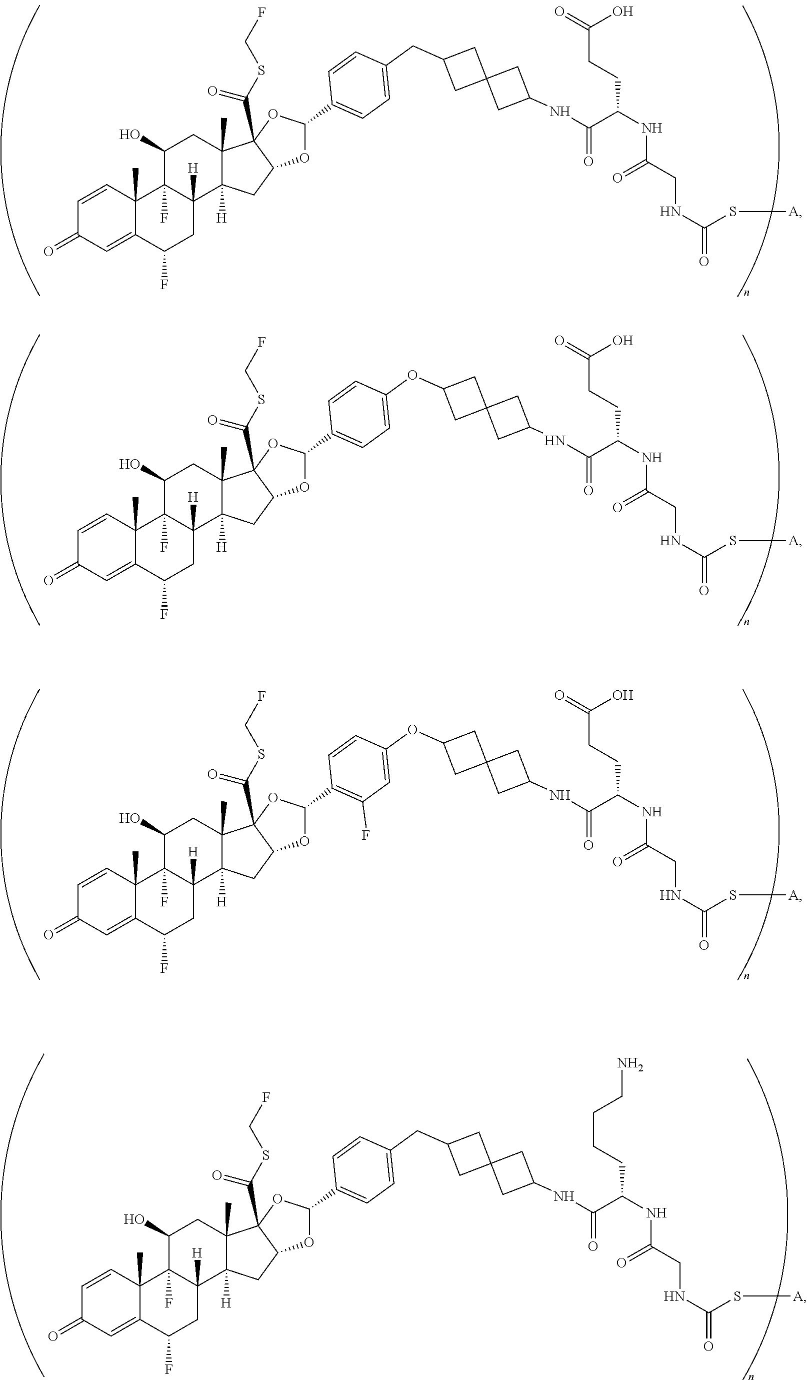 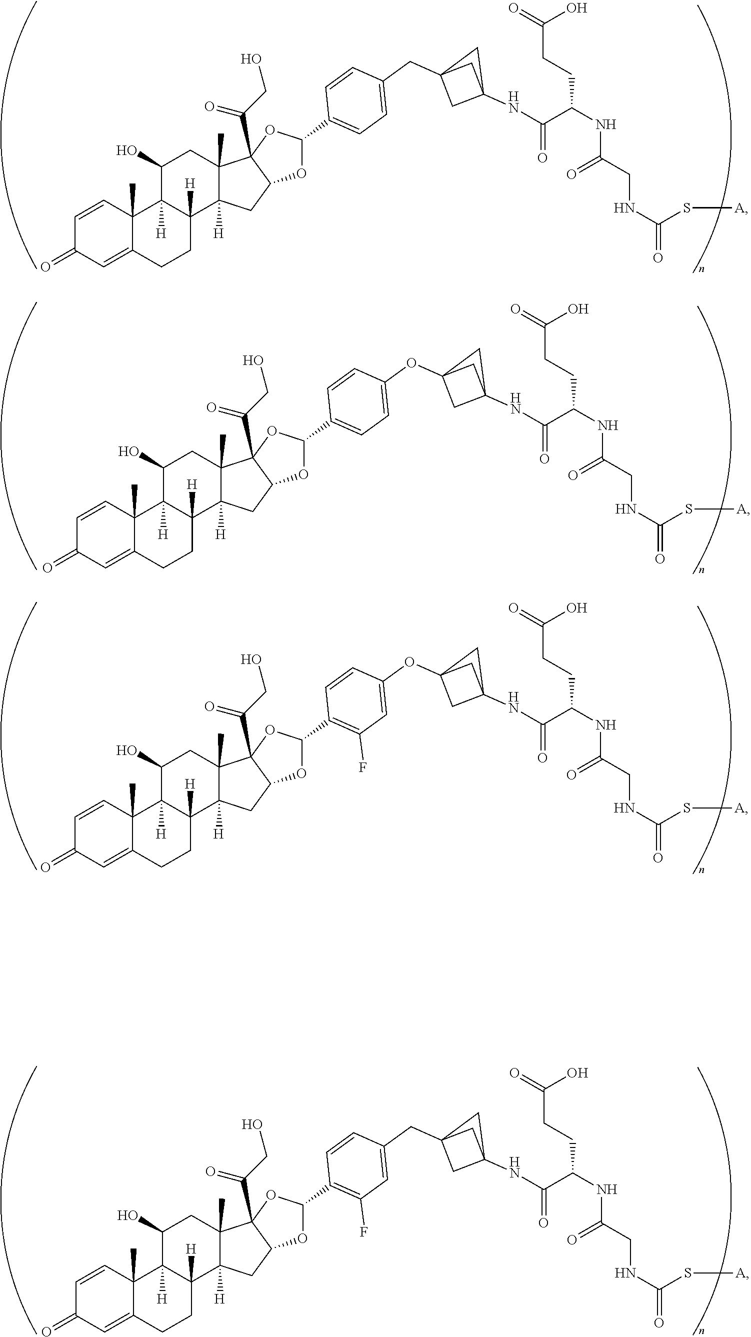 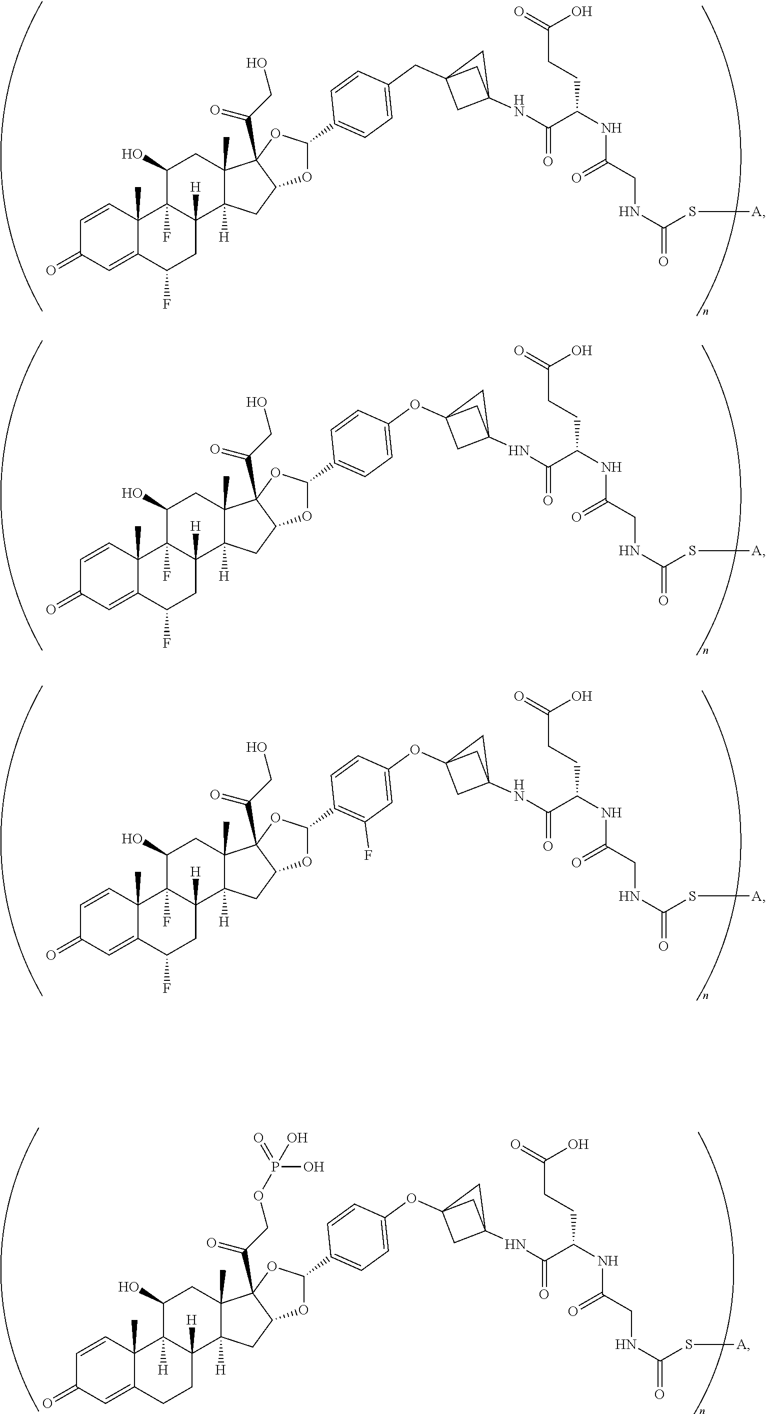 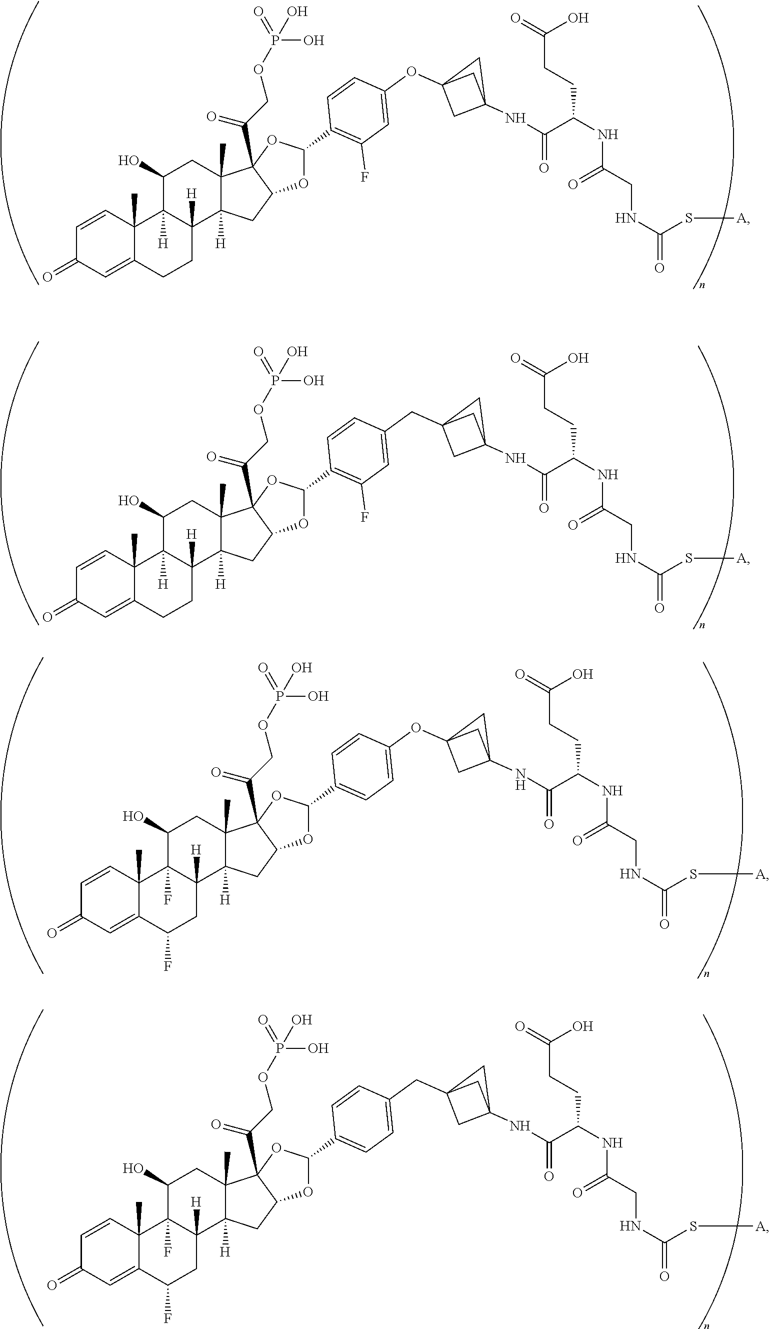 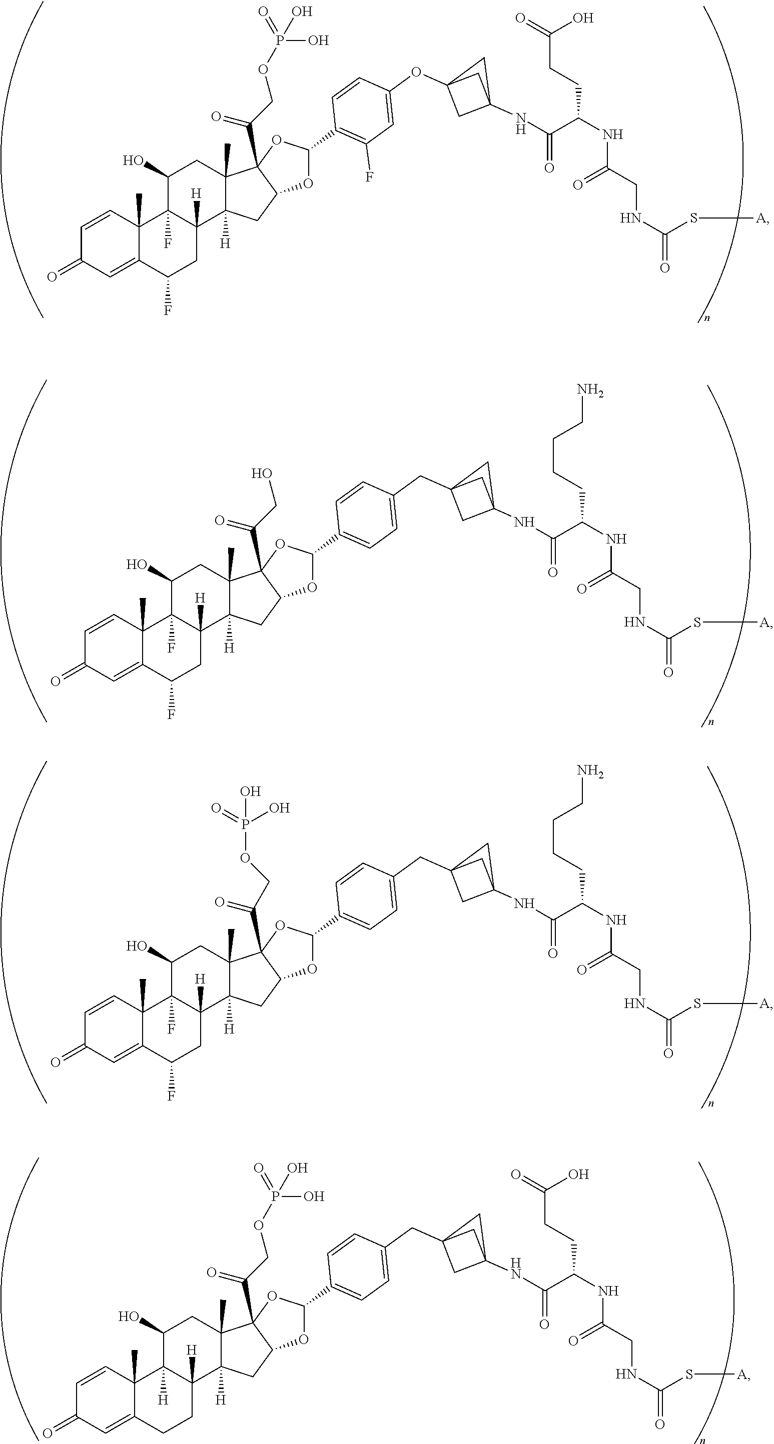 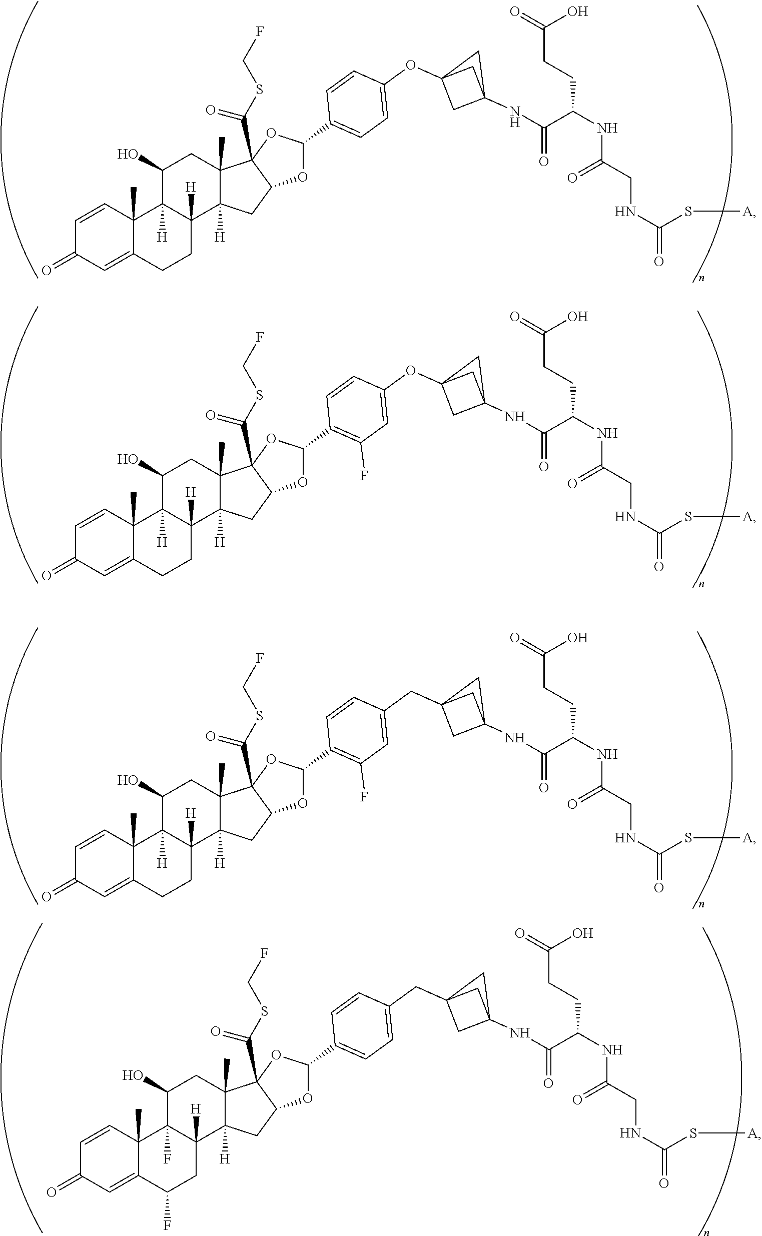 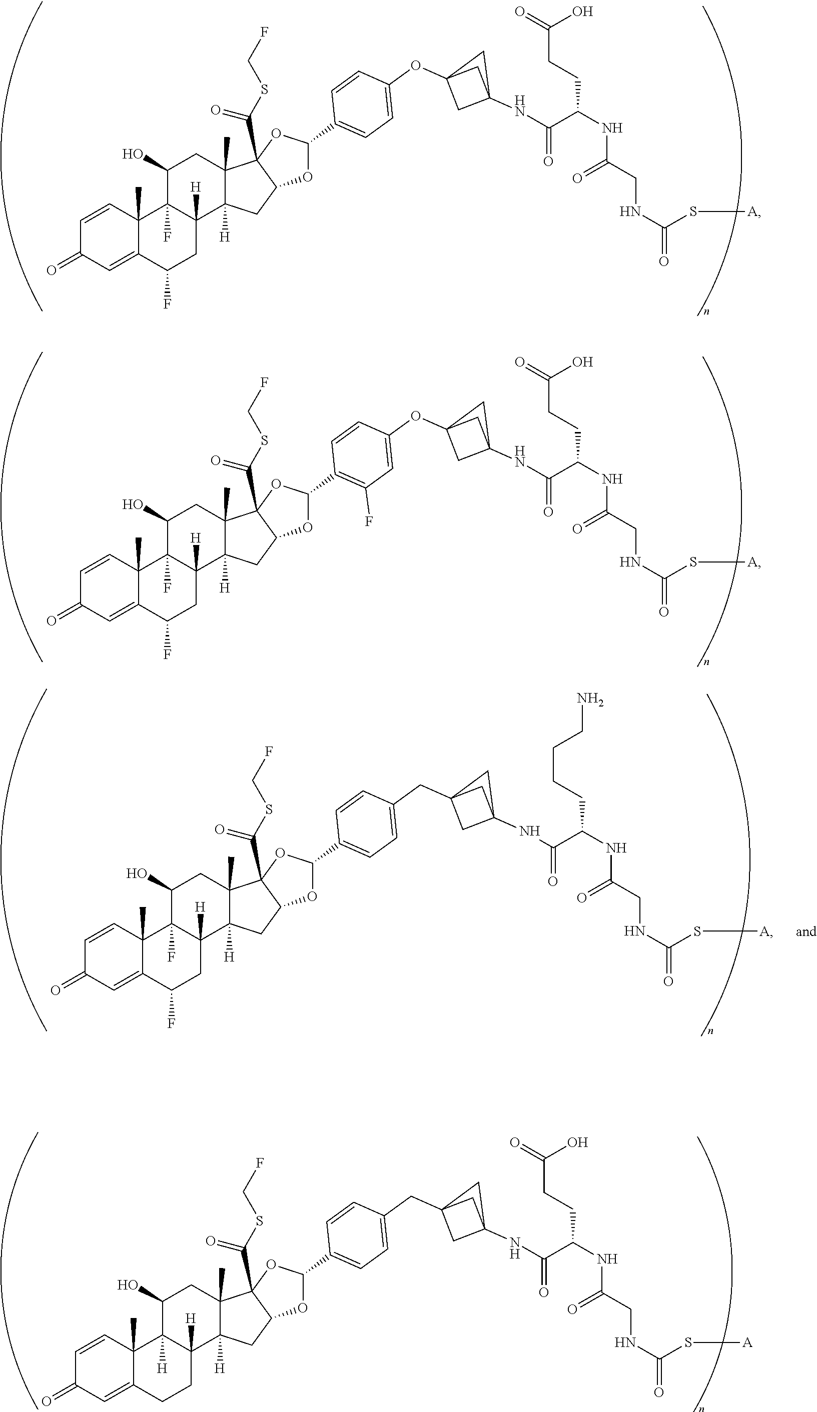
preferably where n=2-12, 2-10, 2-8, 2-6, or 2-4 and A is an antibody which binds to an antigen expressed by an immune cell, preferably a human immune cell and more preferably an anti-human VISTA antibody.

138. A composition comprising
(a) at least one glucocorticoid agonist compound according to claim 133, or a steroid-linker conjugate comprising said at least one glucocorticoid agonist compound attached to a linker or an ADC comprising said glucocorticoid agonist compound according to claim 133, or a steroid-linker conjugate comprising said at least one glucocorticoid agonist compound attached to a linker; and
(b) a pharmaceutically acceptable carrier; which optionally:
(i) is suitable for in vivo administration to a subject in need thereof;
(ii) comprises at least one excipient;
(iii) comprises at least one stabilizer or buffer;
(iv) is suitable for parenteral administration, optionally by injection;
(v) is suitable for injection to a subject in need thereof, optionally via intravenous, subcutaneous, intramuscular, intratumoral, intranodal, intranasal, or intrathecal;
(vi) is subcutaneously administrable;
(vii) is comprised in a device that provides for subcutaneous administration selected from the group consisting of a syringe, an injection device, an infusion pump, an injector pen, a needleless device, an autoinjector, and a subcutaneous patch delivery system; which further optionally delivers to a patient a fixed dose of the glucocorticoid receptor agonist; or a combination of any of the foregoing.

139. A method of treatment or prophylaxis comprising the administration of at least one at least one glucocorticoid agonist compound according to claim 133, or a steroid-linker conjugate comprising said at least one glucocorticoid agonist compound attached to a linker or an ADC comprising said glucocorticoid agonist compound according to claim 133, or a steroid-linker conjugate comprising said at least one glucocorticoid agonist compound attached to a linker; or a composition containing, for:
(i) treating, preventing or inhibiting inflammation or autoimmunity in a subject in need thereof;
(ii) treating or preventing an allergic reaction in a subject in need thereof;
(iii) treatment of allergy, autoimmunity, transplant, gene therapy, inflammation, GVHD or sepsis, or to treat or prevent inflammatory, autoimmune, or allergic side effects associated with any of the foregoing conditions in a human subject;
(iv) treating an acute inflammatory or autoimmune condition;
(v) treating a chronic inflammatory or autoimmune condition;
(vii) maintenance therapy of an inflammatory or autoimmune condition;
(viii) treatment or prophylaxis of acute or chronic inflammation and autoimmune and inflammatory indications associated therewith wherein the conditions optionally include Acquired aplastic anemia+, Acquired hemophilia+, Acute disseminated encephalomyelitis (ADEM)+, Acute hemorrhagic leukoencephalitis (AHLE)/Hurst's disease+, Agammaglobulinemia, primary+, Alopecia areata+, Ankylosing spondylitis (AS), Anti-NMDA receptor encephalitis+, Antiphospholipid syndrome (APS)+, Arteriosclerosis, Autism spectrum disorders (ASD), Autoimmune Addison's disease (AAD)+, Autoimmune dysautonomia/Autoimmune autonomic ganglionopathy (AAG), Autoimmune encephalitis+, Autoimmune gastritis, Autoimmune hemolytic anemia (AIHA)+, Autoimmune hepatitis (AIH)+, Autoimmune hyperlipidemia, Autoimmune hypophysitis/lymphocytic hypophysitis+, Autoimmune inner ear disease (AIED)+, Autoimmune lymphoproliferative syndrome (ALPS)+, Autoimmune myocarditis, Autoimmune oophoritis+, Autoimmune orchitis+, Autoimmune pancreatitis (AIP)/Immunoglobulin G4-Related Disease (IgG4-RD)+, Autoimmune polyglandular syndromes, Types I, II, & III+, Autoimmune progesterone dermatitis+, Autoimmune sudden sensorineural hearing loss (SNHL) Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Diabetes, type 1, Dermatitis herpetiformis, Dermatomyositis, Devic's disease (neuromyelitis optica). Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Fibrosing alveolitis, Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus (including nephritis and cutaneous), Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myelin Oligodendrocyte Glycoprotein Antibody Disorder, Myositis, Narcolepsy, Neonatal Lupus, Neuromyelitis optica, Neutropenia, Ocular cicatricial pemphigoid, Optic neuritis, Opsoclonus-myoclonus syndrome (OMS), Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary Biliary Cholangitis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopeniarpura (TTP), Thyroid eye disease (TED), Tolosa-Hunt syndrome (THS), Transverse myelitis, Type 1 diabetes, Undifferentiated connective tissue disease (UCTD), Uveitis, Vasculitis, Vitiligo, Vogt-Koyanagi-Harada Disease, among others;
(viii) treatment or prophylaxis of acute or chronic inflammation and autoimmune and inflammatory and allergic indications or side-effects associated therewith, wherein the conditions optionally include Severe asthma, Giant cell arteritis, ANKA vasculitis and IBD (Colitis and Crohns);
(ix) treatment or prophylaxis of a condition selected from rheumatoid arthritis, juvenile idiopathic arthritis, psoriatic arthritis, ankylosing spondylitis, adult Crohn's disease, pediatric Crohn's disease, ulcerative colitis, plaque psoriasis, hidradenitis suppurativa, uveitis, Bechet's disease, a spondyloarthropathy, or psoriasis;
(x) treatment or prophylaxis in a patient who comprises one or more of the following:
(a) a chronic, acute, episodic allergic, inflammatory or inflammatory condition, e.g., chronic, acute, episodic, remitting/relapsing;
(b) a condition primarily only effectively treatable with high doses of steroids, optionally polymyalgia rheumatica and/or giant cell arteritis, which patient optionally has been treated or is undergoing treatment with high steroid doses;
(c) a condition with a comorbidity limiting steroid use, optionally diabetes mellitis, nonalcoholic steatohepatitis (NASH), morbid obesity avascular necrosis/osteonecrosis (AVN), glaucoma. Steroid-induced hypertension, severe skin fragility, and/or osteoarthritis;
(d) a condition wherein safe long-term treatment agents are available, but wherein several months of induction with high-doses of steroids is desired, optionally AAV, polymyositis, dermamyositis, lupus, inflammatory lung disease, autoimmune hepatitis, inflammatory bowel disease, immune thrombocytopenia, autoimmune hemolytic anemia, gout patients wherein several months of induction with high-doses of steroids is therapeutically warranted;
(e) dermatologic conditions that require short/long-term treatment, optionally of uncertain treatment or duration and/or no effective alternative to steroid administration, optionally Stevens Johnson, other severe drug eruption conditions, conditions involving extensive contact dermatitis, other severe immune-related dermatological conditions such as PG, LCV, Erythroderma and the like;
(f) conditions treated with high-dose corticosteroids for flares/reoccurrences, optionally COPD, asthma, lupus, gout, pseudogout;
(g) immune-related neurologic diseases such as small-fiber neuropathy, MS (subset), chronic inflammatory demyelinating polyneuropathy, myasthenia gravis and the like;
(h) hematological/oncology indications, optionally wherein high doses of steroids would potentially be therapeutically warranted or beneficial;
(i) ophthalmologic conditions, optionally uveitis, iritis, scleritis, and the like;
(j) conditions associated with permanent or very prolonged adrenal insufficiency or secondary adrenal insufficiency, optionally latrogenic Addisonian crisis;
(k) conditions often treated with long term, low dose steroids, optionally lupus, RA, psA, vasculitis, and the like;
(xi) for use in the treatment or prophylaxis in a patient who is in a special class of patients who are at risk of toxicity in steroid treatment such as pregnant/breast-feeding women, pediatric patients optionally those with growth impairment or cataracts, wherein the patient is further being treated with another active agent;
(xiii) use in treating a patient further being treated with an immunomodulatory antibody or fusion protein which is selected from immmunoinhibitory antibodies or fusion proteins targeting one or more of CTLA4, PD-1, PDL-1, LAG-3, TIM-3, BTLA, B7-H4, B7-H3, VISTA, and/or agonistic antibodies or fusion protein targeting one or more of CD40, CD137, OX40, GITR, CD27, CD28 or ICOS; or a combination of any of the foregoing.

140. An antibody drug conjugate (ADC), according to claim 135, or a composition containing, wherein
(i) the ADC comprises an antibody or antigen binding fragment comprising an antigen binding region that specifically binds to human V-domain Ig Suppressor of T cell Activation (human VISTA) (“A”), wherein the ADC, when administered to a subject in need thereof, is preferentially delivered to VISTA expressing immune cells, optionally one or more of monocytes, myeloid cells, T cells, Tregs, NK cells, Neutrophils, Dendritic cells, macrophages, eosinophils, and endothelial cells, and results in the functional internalization of the anti-inflammatory agent into one or more of said immune cells;
(ii) the ADC comprises an anti-human VISTA antibody or antibody fragment that preferentially binds to VISTA expressing cells at physiological pH (≈7.5); which optionally has a pK of at most 70 hours in a human VISTA knock-in rodent;
(iii) the ADC comprises an anti-human VISTA antibody or antibody fragment, which binds to VISTA expressing cells at physiologic pH, and which has a pK of at most 3.5±0.5 days, more typically at most 48 hours, at most 24 hours, at most 18 hours or at most 12 hours in a Cynomolgus macaque or a human at physiologic pH;
(iv) the ADC comprises an anti-human VISTA antibody or antibody fragment, which has a pk of at most 2.8 or 2.3 or 1.5 days or 1 day or 12 hours or 8 hours±0.5 days in Cynomolgus macaque or in a human at physiologic pH;
(v) the ADC comprises an anti-human VISTA antibody or antibody fragment, which has a pk of at most 6-12 hours in a human VISTA rodent at physiologic pH;
(vi) the ADC comprises an anti-human VISTA antibody or antibody fragment, which comprises a linker which upon internalization of the ADC into VISTA-expressing immune cells, optionally one or more of T cells, Tregs, NK cells, neutrophils, monocytes, myeloid cells, dendritic cells, macrophages, eosinophils, and endothelial cells, is cleaved resulting in the release of a therapeutically effective amount of an anti-inflammatory agent in the immune cell, wherein it elicits anti-inflammatory activity;
(vii) the ADC comprises an anti-human VISTA antibody or antibody fragment the anti-VISTA antibody or antigen binding fragment has an in vivo serum half-life of about 2.3 days in a primate, optionally Cynomolgus macaque at physiological pH (^{˜}pH 7.5);
(viii) the ADC comprises an anti-human VISTA antibody or antibody fragment, wherein the anti-VISTA antibody or antigen binding fragment has an in vivo serum half-life in serum at physiological pH (^{˜}pH 7.5) in a human VISTA knock-in rodent of no more than 70 hours, no more than 60 hours, no more than 50 hours, no more than 40 hours, no more than 30 hours, no more than 24 hours, no more than 22-24 hours, no more than 20-22 hours, no more than 18-20 hours, no more than 16-18 hours, no more than 14-16 hours, no more than 12-14 hours, no more than 10-12 hours, no more than 8-10 hours, no more than 6-8 hours, no more than 4-6 hours, no more than 2-4 hours, no more than 1-2 hours, no more than 0.5 to 1.0 hours, or no more than 0.1-0.5 hours;
(ix) the ADC comprises an anti-human VISTA antibody or antibody fragment, wherein the PD/PK ratio of the ADC when used in vivo is at least 2:1, 3:1, 4:1, 5:1, 6:1, 7:1, 8:1, 9:1, 10:1, 11:1, 12:1, 13:1, 14:1, 28:1 or greater in a human VISTA knock-in rodent or in a human or non-human primate, optionally Cynomolgus macaque;
(x) the ADC comprises an anti-human VISTA antibody or antibody fragment, wherein the PD of the ADC is at least 4, 5, 6, 7, 8, 9, 10, 11, 12, 13, 14 or 28 days, 2-3 weeks, 1 month, 2 months, or longer in any one of a rodent or in a human or non-human primate, optionally Cynomolgus macaque;
(xi) the ADC comprises an anti-human VISTA antibody or antibody fragment, wherein the anti-human VISTA antibody comprises an Fc region having impaired FcR binding or intact FcR binding;
(xii) the ADC comprises an antibody or antibody fragment, optionally an anti-human VISTA antibody or antibody fragment, wherein the antibody or antibody fragment comprises a human IgG1, IgG2, IgG3 or IgG4 Fc region having impaired FcR binding or intact FcR binding;
(xiii) the ADC comprises an antibody or antibody fragment, optionally an anti-human VISTA antibody or antibody fragment wherein the antibody or antibody fragment comprises a human IgG1 Fc region having impaired FcR binding;
(xiv) the ADC comprises an antibody or antibody fragment, optionally an anti-human VISTA antibody or antibody fragment, comprising a human or non-human primate constant or Fc region which is modified to impair or eliminate binding to at least 2 native human Fc gamma receptors;
(xv) the ADC comprises an antibody or antibody fragment, optionally an anti-human VISTA antibody or antibody fragment, comprising a human or non-human primate constant or Fc region modified to impair or eliminate binding to any one, two, three, four or all five of the following FcRs: hFcγRI(CD64), FcyRIIA or hFcyRIIB, (CD32 or CD32A) and FcγRIIIA (CD16A) or FcγRIIIB (CD16B);
(xvi) the ADC comprises an antibody or antibody fragment, optionally an anti-human VISTA antibody or antibody fragment, comprising a human IgG2 kappa backbone, optionally with V234A/G237A/P238S/H268A/V309L/A330S/P331S silencing mutations in the Fc region;
(xvii) the ADC comprises an antibody or antibody fragment, optionally an anti-human VISTA antibody or antibody fragment, optionally a human IgG1/kappa backbone with L234A/L235A silencing mutations in the Fc region and optionally a mutation which impairs complement (C1Q) binding;
(xviii) the ADC comprises an antibody or antibody fragment, optionally an anti-human VISTA antibody or antibody fragment, comprising a human IgG1/kappa backbone, optionally with L234A/L235A silencing mutations and E269R and E233A mutations in the Fc region;
(xix) the ADC comprises an anti-human VISTA antibody or antibody fragment wherein the binding of the anti-VISTA antibody or antigen binding fragment to VISTA expressing immune cells does not directly agonize or antagonize VISTA-mediated effects on immunity;
(xx) the ADC comprises an antibody or antibody fragment, optionally an anti-human VISTA antibody or antibody fragment, comprising a human IgG1, IgG2, IgG3 or IgG4 Fc region wherein endogenous FcR binding is not impaired;
(xxi) the ADC comprises an antibody or antibody fragment, optionally an anti-human VISTA antibody or antibody fragment, comprising a native (unmodified) human IgG2 Fc region;
(xxii) the ADC comprises an antibody or antibody fragment, optionally an anti-human VISTA antibody or antibody fragment, wherein the antibody or antigen binding fragment comprises a KD ranging from 0.0001 nM to 10.0 nM, 0.001 to 1.0 nM, or 0.01 to 0.7 or less determined by surface plasmon resonance (SPR) at 24° C. or 37° C.;
(xxiii) the ADC comprises an antibody or antibody fragment, optionally an anti-human VISTA antibody or antibody fragment, wherein the antibody or antigen binding fragment comprises a KD of 0.13 to 0.64 nM determined by surface plasmon resonance (SPR) at 24° C. or 37° C.;
(xxiv) the ADC optionally comprises an antibody or antibody fragment, optionally an anti-human VISTA antibody or antibody fragment, wherein the drug antibody ratio ranges from 1:1-12:1;
(xxv) the ADC optionally comprises an antibody or antibody fragment, optionally an anti-human VISTA antibody or antibody fragment, wherein the drug antibody ratio ranges from 2-12:1, 2-8:1, 4-8:1, or 6-8:1;
(xxvi) the ADC comprises optionally an anti-human VISTA antibody or antibody fragment, wherein the drug antibody ratio the drug antibody ratio is about 8:1 (n=8) or is about 4:1 (n=4);
(xxvii) the ADC comprises an antibody or antibody fragment, optionally an anti-human VISTA antibody or antibody fragment, which internalizes one or more of monocytes, myeloid cells, T cells, Tregs, CD4 T cells, CD8 T cells, macrophages, NK cells, macrophages, eosinophils, mast cells, B cells, and neutrophils;
(xxviii) the ADC comprises an antibody or antibody fragment, optionally an anti-human VISTA antibody or antibody fragment, which does not appreciably internalize B cells;
(xxix) ADC comprises an antibody or antibody fragment, optionally an anti-human VISTA antibody or antibody fragment, which when administered to a subject in need thereof promotes the efficacy and/or reduces adverse side effects such as toxicity associated with the anti-inflammatory agent, compared to the same dosage of anti-inflammatory agent administered in naked (non-conjugated) form;
(xxx) the ADC comprises an antibody or antibody fragment, optionally an anti-human VISTA antibody or antibody fragment, wherein the glucocorticoid is optionally conjugated to the antibody or antigen-binding fragment via the interchain disulfides;
(xxxi) the ADC comprises an antibody or antibody fragment, optionally an anti-human VISTA antibody or antibody fragment, which comprises an esterase sensitive linker;
(xxxii) the ADC comprises an antibody or antibody fragment, optionally an anti-human VISTA antibody or antibody fragment, wherein the cleavable linker is susceptible to one or more of acid-induced cleavage, photo-induced cleavage, peptidase-induced cleavage, esterase-induced cleavage, and disulfide bond cleavage;
(xxxiii) the ADC comprises an antibody or antibody fragment, optionally an anti-human VISTA antibody or antibody fragment, wherein the antigen binding fragment comprised in the ADC comprises a Fab, F(ab′)2, or scFv antibody fragment;
(xxxiv) the ADC comprises an anti-human VISTA antibody or antibody fragment, wherein the anti-VISTA antibody or antibody fragment contained therein is one which comprises the same CDRs as an antibody having the sequences in FIG. 8, 10 or 12 or is optionally selected from one that:
(i) comprises the V_{H} CDRs of SEQ ID NO:100, 101 and 102 and the V_{L} CDRs of SEQ ID NO: 103, 104 and 105;
(ii) comprises the V_{H} CDRs of SEQ ID NO:110, 111 and 112 and the V_{L} CDRs of SEQ ID NO: 113, 114 and 115;
(iii) comprises the V_{H} CDRs of SEQ ID NO: 120, 121 and 122 and the V_{L} CDRs of SEQ ID NO: 123, 124 and 125;
(iv) comprises the V_{H} CDRs of SEQ ID NO: 130, 131 and 132 and the V_{L} CDRs of SEQ ID NO: 133, 134 and 135;
(v) comprises the V_{H} CDRs of SEQ ID NO: 140, 141 and 142 and the V_{L} CDRs of SEQ ID NO: 143, 144 and 145;
(vi) comprises the V_{H} CDRs of SEQ ID NO: 150, 151 and 152 and the V_{L} CDRs of SEQ ID NO: 153, 154 and 155;
(vii) comprises the V_{H} CDRs of SEQ ID NO: 160, 161 and 162 and the V_{L} CDRs of SEQ ID NO: 163, 164 and 165;
(viii) comprises the V_{H} CDRs of SEQ ID NO: 170, 171 and 172 and the V_{L} CDRs of SEQ ID NO: 173, 174 and 175;
(ix) comprises the V_{H} CDRs of SEQ ID NO: 180, 181 and 182 and the V_{L} CDRs of SEQ ID NO: 183, 184 and 185;
(x) comprises the V_{H} CDRs of SEQ ID NO: 190, 191 and 192 and the V_{L} CDRs of SEQ ID NO: 193, 194 and 195;
(xi) comprises the V_{H} CDRs of SEQ ID NO:200, 201 and 202 and the V_{L} CDRs of SEQ ID NO: 203, 204 and 205;
(xii) comprises the V_{H} CDRs of SEQ ID NO:210, 211 and 212 and the V_{L} CDRs of SEQ ID NO: 213, 214 and 215;
(xiii) comprises the V_{H} CDRs of SEQ ID NO:220, 221 and 222 and the V_{L} CDRs of SEQ ID NO: 223, 224 and 225;
(xiv) comprises the V_{H} CDRs of SEQ ID NO: 230, 231 and 232 and the V_{L} CDRs of SEQ ID NO: 233, 234 and 235;
(xv) comprises the V_{H} CDRs of SEQ ID NO:240, 241 and 242 and the V_{L} CDRs of SEQ ID NO: 243, 244 and 245;
(xvi) comprises the V_{H} CDRs of SEQ ID NO: 250, 251 and 252 and the V_{L} CDRs of SEQ ID NO: 253, 254 and 255;
(xvii) comprises the V_{H} CDRs of SEQ ID NO: 260, 261 and 262 and the V_{L} CDRs of SEQ ID NO: 263, 264 and 265;
(xviii) comprises the V_{H} CDRs of SEQ ID NO:270, 271 and 272 and the V_{L} CDRs of SEQ ID NO: 273, 274 and 275;
(xix) comprises the V_{H} CDRs of SEQ ID NO: 280, 281 and 282 and the V_{L} CDRs of SEQ ID NO: 283, 284 and 285;
(xx) comprises the V_{H} CDRs of SEQ ID NO: 290, 291 and 292 and the V_{L} CDRs of SEQ ID NO: 293, 294 and 295;
(xxi) comprises the V_{H} CDRs of SEQ ID NO:300, 301 and 302 and the V_{L} CDRs of SEQ ID NO: 303, 304 and 305;
(xxii) comprises the V_{H} CDRs of SEQ ID NO:310, 311 and 312 and the V_{L} CDRs of SEQ ID NO: 313, 314 and 315;
(xxiii) comprises the V_{H} CDRs of SEQ ID NO:320, 321 and 322 and the V_{L} CDRs of SEQ ID NO: 323, 324 and 325;
(xxiv) comprises the V_{H} CDRs of SEQ ID NO:330, 331 and 332 and the V_{L} CDRs of SEQ ID NO: 333, 334 and 335;
(xxv) comprises the V_{H} CDRs of SEQ ID NO: 340, 341 and 342 and the V_{L} CDRs of SEQ ID NO: 343, 344 and 345;
(xxvi) comprises the V_{H} CDRs of SEQ ID NO:350, 351 and 352 and the V_{L} CDRs of SEQ ID NO: 353, 354 and 355;
(xxvii) comprises the V_{H} CDRs of SEQ ID NO:360, 361 and 362 and the V_{L} CDRs of SEQ ID NO: 363, 364 and 365;
(xxviii) comprises the V_{H} CDRs of SEQ ID NO:370, 371 and 372 and the V_{L} CDRs of SEQ ID NO: 373, 374 and 375;
(xxix) comprises the V_{H} CDRs of SEQ ID NO:380, 381 and 382 and the V_{L} CDRs of SEQ ID NO: 383, 384 and 385;
(xxx) comprises the V_{H} CDRs of SEQ ID NO:390, 391 and 392 and the V_{L} CDRs of SEQ ID NO: 393, 394 and 395;
(xxxi) comprises the V_{H} CDRs of SEQ ID NO:400, 401 and 402 and the V_{L} CDRs of SEQ ID NO: 403, 404 and 405;
(xxxii) comprises the V_{H} CDRs of SEQ ID NO:410, 411 and 412 and the V_{L} CDRs of SEQ ID NO: 413, 414 and 415;
(xxxiii) comprises the V_{H} CDRs of SEQ ID NO:420, 421 and 422 and the V_{L} CDRs of SEQ ID NO: 423, 424 and 425;
(xxxiv) comprises the V_{H} CDRs of SEQ ID NO:430, 431 and 432 and the V_{L} CDRs of SEQ ID NO: 433, 434 and 435;
(xxxv) comprises the V_{H} CDRs of SEQ ID NO:440, 441 and 442 and the V_{L} CDRs of SEQ ID NO: 443, 444 and 445;
(xxxvi) comprises the V_{H} CDRs of SEQ ID NO:450, 451 and 452 and the V_{L} CDRs of SEQ ID NO: 453, 454 and 455;
(xxxvii) comprises the V_{H} CDRs of SEQ ID NO:460, 461 and 462 and the V_{L} CDRs of SEQ ID NO: 463, 464 and 465;
(xxxviii) comprises the V_{H} CDRs of SEQ ID NO:470, 471 and 472 and the V_{L} CDRs of SEQ ID NO: 473, 474 and 475;
(xxxix) comprises the V_{H} CDRs of SEQ ID NO:480, 481 and 482 and the V_{L} CDRs of SEQ ID NO: 483, 484 and 485;
(xl) comprises the V_{H} CDRs of SEQ ID NO:490, 491 and 492 and the V_{L} CDR polypeptides of SEQ ID NO:493, 494 and 495;
(xli) comprises the V_{H} CDRs of SEQ ID NO:500, 501 and 502 and the V_{L} CDR polypeptides of SEQ ID NO:503, 504 and 505;
(xlii) comprises the V_{H} CDRs of SEQ ID NO:510, 511 and 512 and the V_{L} CDR polypeptides of SEQ ID NO:513, 514 and 515;
(xliii) comprises the V_{H} CDRs of SEQ ID NO:520, 521 and 522 and the V_{L} CDR polypeptides of SEQ ID NO:523, 524 and 525;
(xliv) comprises the V_{H} CDRs of SEQ ID NO:530, 531 and 532 and the V_{L} CDR polypeptides of SEQ ID NO:533, 534 and 535;
(xlv) comprises the V_{H} CDRs of SEQ ID NO:540, 541 and 542 and the V_{L} CDR polypeptides of SEQ ID NO:543, 544 and 545;
(xlvi) comprises the V_{H} CDRs of SEQ ID NO:550, 551 and 552 and the V_{L} CDR polypeptides of SEQ ID NO:553, 554 and 555;
(xlvii) comprises the V_{H} CDRs of SEQ ID NO:560, 561 and 562 and the V_{L} CDRs of SEQ ID NO: 563, 564 and 565;
(xlviii) comprises the V_{H} CDRs of SEQ ID NO:570, 571 and 572 and the V_{L} CDRs of SEQ ID NO: 573, 574 and 575;
(xlix) comprises the V_{H} CDRs of SEQ ID NO:580, 581 and 582 and the V_{L} CDRs of SEQ ID NO: 583, 584 and 585;
(li) comprises the V_{H} CDRs of SEQ ID NO:590, 591 and 592 and the V_{L} CDRs of SEQ ID NO: 593, 594 and 595;
(li) comprises the V_{H} CDRs of SEQ ID NO: 600, 601 and 602 and the V_{L} CDRs of SEQ ID NO: 603, 604 and 605;
(lii) comprises the V_{H} CDRs of SEQ ID NO:610, 611 and 612 and the V_{L} CDRs of SEQ ID NO: 613, 614 and 615;
(liii) comprises the V_{H} CDRs of SEQ ID NO:620, 621 and 622 and the V_{L} CDRs of SEQ ID NO: 623, 624 and 625;
(liv) comprises the V_{H} CDRs of SEQ ID NO:630, 631 and 632 and the V_{L} CDRs of SEQ ID NO: 633, 634 and 635;
(lv) comprises the V_{H} CDRs of SEQ ID NO:640, 641 and 642 and the V_{L} CDRs of SEQ ID NO: 643, 644 and 645;
(lvi) comprises the V_{H} CDRs of SEQ ID NO:650, 651 and 652 and the V_{L} CDRs of SEQ ID NO: 653, 654 and 655;
(lvii) comprises the V_{H} CDRs of SEQ ID NO: 660, 661 and 662 and the V_{L} CDRs of SEQ ID NO: 663, 664 and 665;
(lviii) comprises the V_{H} CDRs of SEQ ID NO:670, 671 and 672 and the V_{L} CDRs of SEQ ID NO: 673, 674 and 675;
(lix) comprises the V_{H} CDRs of SEQ ID NO:680, 681 and 682 and the V_{L} CDRs of SEQ ID NO: 683, 684 and 685;
(lx) comprises the V_{H} CDRs of SEQ ID NO: 690, 691 and 692 and the V_{L} CDRs of SEQ ID NO: 693, 694 and 695;
(lxi) comprises the V_{H} CDRs of SEQ ID NO: 700, 701 and 702 and the V_{L} CDRs of SEQ ID NO: 703, 704 and 705;
(lxii) comprises the V_{H} CDRs of SEQ ID NO: 710, 711 and 712 and the V_{L} CDRs of SEQ ID NO: 713, 714 and 715;
(lxiii) comprises the V_{H} CDRs of SEQ ID NO: 720, 721 and 722 and the V_{L} CDRs of SEQ ID NO: 723, 724 and 725;
(lxiv) comprises the V_{H} CDRs of SEQ ID NO: 730, 731 and 732 and the V_{L} CDRs of SEQ ID NO: 733, 734 and 735;
(lxv) comprises the V_{H} CDRs of SEQ ID NO: 740, 741 and 742 and the V_{L} CDRs of SEQ ID NO: 743, 744 and 745;
(lxvi) comprises the V_{H} CDRs of SEQ ID NO: 750, 751 and 752 and the V_{L} CDRs of SEQ ID NO: 753, 754 and 755;
(lxvii) comprises the V_{H} CDRs of SEQ ID NO:760, 761 and 762 and the V_{L} CDRs of SEQ ID NO: 763, 764 and 765;
(lxviii) comprises the V_{H} CDRs of SEQ ID NO: 770, 771 and 772 and the V_{L} CDRs of SEQ ID NO: 773, 774 and 775;
(xix) comprises the V_{H} CDRs of SEQ ID NO: 780, 781 and 782 and the V_{L} CDRs of SEQ ID NO: 783, 784 and 785;
(lxx) comprises the V_{H} CDRs of SEQ ID NO: 790, 791 and 792 and the V_{L} CDRs of SEQ ID NO: 793, 794 and 795;
(lxxi) comprises the V_{H} CDRs of SEQ ID NO:800, 801 and 802 and the V_{L} CDRs of SEQ ID NO: 803, 804 and 805;
(lxxii) comprises the V_{H} CDRs of SEQ ID NO:810, 811 and 812 and the V_{L} CDRs of SEQ ID NO: 813, 814 and 815;
(xxxv) the ADC comprises an anti-VISTA antibody or antibody fragment that comprises the same CDRS as any one of VSTB92, VSTB56, VSTB95, VSTB103 and VSTB66;
(xxxvi) the ADC comprises an anti-VISTA antibody or antibody fragment that comprises a V_{H} polypeptide and a V_{L} polypeptide which respectively possess at least 90%, 95% or 100% sequence identity to those of an antibody comprising the following V_{H} polypeptide and a V_{L} polypeptides and further the CDRs are not modified:
(i) one comprising the V_{H} polypeptide of SEQ ID NO:106 identity and the V_{L} polypeptide of SEQ ID NO:108;
(ii) one comprising the V_{H} polypeptide of SEQ ID NO:116 and the V_{L} polypeptide of SEQ ID NO: 118;
(iii) one comprising the V_{H} polypeptide of SEQ ID NO:126 and the V_{L} polypeptide of SEQ ID NO: 128;
(iv) one comprising the V_{H} polypeptide of SEQ ID NO: 136 and the V_{L} polypeptide f SEQ ID NO: 138;
(v) one comprising the V_{H} polypeptide of SEQ ID NO:146 and the V_{L} polypeptide of SEQ ID NO: 148;
(vi) one comprising the V_{H} polypeptide of SEQ ID NO:156 and the V_{L} polypeptide of SEQ ID NO: 158;
(vii) one comprising the V_{H} polypeptide of SEQ ID NO: 166 and the V_{L} polypeptide of SEQ ID NO: 168;
(viii) one comprising the V_{H} polypeptide of SEQ ID NO:176 and the V_{L} polypeptide of SEQ ID NO: 178;
(ix) one comprising the V_{H} polypeptide of SEQ ID NO: 186 and the V_{L} polypeptide of SEQ ID NO: 188;
(x) one comprising the V_{H} polypeptide of SEQ ID NO: 196 and the V_{L} polypeptide of SEQ ID NO: 198;
(xi) one comprising the V_{H} polypeptide of SEQ ID NO:206 and the V_{L} polypeptide of SEQ ID NO: 208;
(xii) one comprising the V_{H} polypeptide of SEQ ID NO: 216 and the V_{L} polypeptide of SEQ ID NO: 218;
(xiii) one comprising the V_{H} polypeptide of SEQ ID NO:226 and the V_{L} polypeptide of SEQ ID NO: 228;
(xiv) one comprising the V_{H} polypeptide of SEQ ID NO: 236 and the V_{L} polypeptide of SEQ ID NO: 238;
(xv) one comprising the V_{H} polypeptide of SEQ ID NO:246 and the V_{L} polypeptide of SEQ ID NO: 248;
(xvi) one comprising the V_{H} polypeptide of SEQ ID NO:256 and the V_{L} polypeptide of SEQ ID NO: 258;
(xvii) one comprising the V_{H} polypeptide of SEQ ID NO:266 and the V_{L} polypeptide of SEQ ID NO: 268;
(xviii) one comprising the V_{H} polypeptide of SEQ ID NO:276 and the V_{L} polypeptide of SEQ ID NO: 278;
(xix) one comprising the V_{H} polypeptide of SEQ ID NO:286 and the V_{L} polypeptide of SEQ ID NO: 288;
(xx) one comprising the V_{H} polypeptide of SEQ ID NO: 296 and the V_{L} polypeptide of SEQ ID NO: 298;
(xxi) one comprising the V_{H} polypeptide of SEQ ID NO:306 and the V_{L} polypeptide of SEQ ID NO: 308;
(xxii) one comprising the V_{H} polypeptide of SEQ ID NO:316 and the V_{L} polypeptide of SEQ ID NO: 318;
(xxiii) one comprising the V_{H} polypeptide of SEQ ID NO:326 and the V_{L} polypeptide of SEQ ID NO: 328;
(xxiv) one comprising the V_{H} polypeptide of SEQ ID NO:336 and the V_{L} polypeptide of SEQ ID NO: 338;
(xxv) one comprising the V_{H} polypeptide of SEQ ID NO:346 and the V_{L} polypeptide of SEQ ID NO: 348;
(xxvi) one comprising the V_{H} polypeptide of SEQ ID NO:356 and the V_{L} polypeptide of SEQ ID NO: 358;
(xxvii) one comprising the V_{H} polypeptide of SEQ ID NO:366 and the V_{L} polypeptide of SEQ ID NO: 368;
(xxviii) one comprising the V_{H} polypeptide of SEQ ID NO:376 and the V_{L} polypeptide of SEQ ID NO: 378;
(xxix) one comprising the V_{H} polypeptide of SEQ ID NO:386 and the V_{L} polypeptide of SEQ ID NO: 388;
(xxx) one comprising the V_{H} polypeptide of SEQ ID NO:396 and the V_{L} polypeptide of SEQ ID NO: 398;
(xxxi) one comprising the V_{H} polypeptide of SEQ ID NO:406 and the V_{L} polypeptide of SEQ ID NO: 408;
(xxxii) one comprising the V_{H} polypeptide of SEQ ID NO:416 and the V_{L} polypeptide of SEQ ID NO: 418;
(xxxiii) one comprising the V_{H} polypeptide of SEQ ID NO:426 and the V_{L} polypeptide of SEQ ID NO: 428;
(xxxiv) one comprising the V_{H} polypeptide of SEQ ID NO:436 and the V_{L} polypeptide of SEQ ID NO: 438;
(xxxv) one comprising the V_{H} polypeptide of SEQ ID NO:446 and the V_{L} polypeptide of SEQ ID NO: 448;
(xxxvi) one comprising the V_{H} polypeptide of SEQ ID NO:456 and the V_{L} polypeptide of SEQ ID NO: 458;
(xxxvii) one comprising the V_{H} polypeptide of SEQ ID NO:466 and the V_{L} polypeptide of SEQ ID NO: 468;
(xxxviii) one comprising the V_{H} polypeptide of SEQ ID NO:476 and the V_{L} polypeptide of SEQ ID NO:478;
(xxxix) one comprising the V_{H} polypeptide of SEQ ID NO:486 and the V_{L} polypeptide of SEQ ID NO: 488;
(xl) one comprising the V_{H} polypeptide of SEQ ID NO:496 and the V_{L} polypeptide of SEQ ID NO: 498;
(xli) one comprising the V_{H} polypeptide of SEQ ID NO:506 and the V_{L} polypeptide of SEQ ID NO: 508;
(xlii) one comprising the V_{H} polypeptide of SEQ ID NO:516 and the V_{L} polypeptide of SEQ ID NO: 518;
(xliii) one comprising the V_{H} polypeptide of SEQ ID NO:526 and the V_{L} polypeptide of SEQ ID NO: 528;
(xliv) one comprising the V_{H} polypeptide of SEQ ID NO:536 and the V_{L} polypeptide of SEQ ID NO: 533, 534 and 535;
(xlv) one comprising the V_{H} polypeptide of SEQ ID NO:546 and the V_{L} polypeptide of SEQ ID NO: 548;
(xlvi) one comprising the V_{H} polypeptide of SEQ ID NO:556 and the V_{L} polypeptide of SEQ ID NO: 558;
(xlvii) one comprising the V_{H} polypeptide of SEQ ID NO:566 and the V_{L} polypeptide of SEQ ID NO: 568;
(xlviii) one comprising the V_{H} polypeptide of SEQ ID NO:576 and the V_{L} polypeptide of SEQ ID NO: 578;
(xlix) one comprising the V_{H} polypeptide of SEQ ID NO:586 and the V_{L} polypeptide of SEQ ID NO: 588;
(I) one comprising the V_{H} polypeptide of SEQ ID NO:596 and the V_{L} polypeptide of SEQ ID NO: 598;
(li) one comprising the V_{H} polypeptide of SEQ ID NO:606 and the V_{L} polypeptide of SEQ ID NO: 608;
(lii) one comprising the V_{H} polypeptide of SEQ ID NO: 616 and the V_{L} polypeptide of SEQ ID NO: 618;
(liii) one comprising the V_{H} polypeptide of SEQ ID NO:626 and the V_{L} polypeptide of SEQ ID NO: 628;
(liv) one comprising the V_{H} polypeptide of SEQ ID NO:636 and the V_{L} polypeptide of SEQ ID NO: 638;
(lv) one comprising the V_{H} polypeptide of SEQ ID NO: 646 and the V_{L} polypeptide of SEQ ID NO: 648;
(lvi) one comprising the V_{H} polypeptide of SEQ ID NO:656 and the V_{L} polypeptide of SEQ ID NO: 658;
(lvii) one comprising the V_{H} polypeptide of SEQ ID NO:666 and the V_{L} polypeptide of SEQ ID NO: 668;
(lviii) one comprising the V_{H} polypeptide of SEQ ID NO: 676 and the V_{L} polypeptide of SEQ ID NO: 678;
(lix) one comprising the V_{H} polypeptide of SEQ ID NO:686 and the V_{L} polypeptide of SEQ ID NO: 688;
(lx) one comprising the V_{H} polypeptide of SEQ ID NO:696 and the V_{L} polypeptide of SEQ ID NO: 698;
(lxi) one comprising the V_{H} polypeptide of SEQ ID NO: 706 and the V_{L} polypeptide of SEQ ID NO: 708;
(lxii) one comprising the V_{H} polypeptide of SEQ ID NO: 716 and the V_{L} polypeptide of SEQ ID NO: 718;
(lxiii) one comprising the V_{H} polypeptide of SEQ ID NO: 726 and the V_{L} polypeptide of SEQ ID NO: 728;
(lxiv) one comprising the V_{H} polypeptide of SEQ ID NO:736 and the V_{L} polypeptide of SEQ ID NO: 738;
(lxv) one comprising the V_{H} polypeptide of SEQ ID NO: 746 and the V_{L} polypeptide of SEQ ID NO: 748;
(lxvi) one comprising the V_{H} polypeptide of SEQ ID NO: 756 and the V_{L} polypeptide of SEQ ID NO: 758;
(lxvii) one comprising the V_{H} polypeptide of SEQ ID NO: 766 and the V_{L} polypeptide of SEQ ID NO: 768;
(lxviii) one comprising the V_{H} polypeptide of SEQ ID NO:776 and the V_{L} polypeptide of SEQ ID NO: 778;
(lxix) one comprising the V_{H} polypeptide of SEQ ID NO: 786 and the V_{L} polypeptide of SEQ ID NO: 788;
(lxx) one comprising the V_{H} polypeptide of SEQ ID NO: 796 and the V_{L} polypeptide of SEQ ID NO: 798;
(lxxi) one comprising the V_{H} polypeptide of SEQ ID NO: 806 and the V_{L} polypeptide of SEQ ID NO: 808; and
(lxxii) one comprising the V_{H} polypeptide of SEQ ID NO:816 and the V_{L} polypeptide of SEQ ID NO: 818;
(lxxiii) the ADC comprises an anti-human VISTA antibody or antibody fragment, wherein the anti-VISTA antibody or antibody fragment comprises the same variable regions as one of VSTB92, VSTB56, VSTB95, VSTB103 and VSTB66;
(lxxiv) the ADC comprises an anti-human VISTA antibody or antibody fragment, optionally having CDR or variable sequences of one in FIG. 8, 10 or 12, wherein the anti-VISTA antibody or antibody fragment comprises a human IgG2 kappa backbone with V234A/G237A/P238S/H268A/V309L/A330S/P331S silencing mutations in the Fc region;
(lxxv) the ADC comprises an anti-human VISTA antibody or antibody fragment, optionally having CDR or variable sequences of one in FIG. 8, 10 or 12, wherein the anti-VISTA antibody or antibody fragment comprises a human IgG1/kappa backbone with L234A/L235A silencing mutations in the Fc region;
(lxxvi) (xl) the glucocorticosteroid agonist or glucocorticosteroid agonist linker conjugate is conjugated to an antibody or antibody fragment in the ADC, optionally an anti-human VISTA antibody or antibody fragment, via its interchain disulfides; or any combination of the foregoing.

141. A pharmaceutical composition comprising a therapeutically effective amount of at least one antibody drug conjugate (ADC) or steroid agonist according to claim 133, at least one steroid-linker comprising said steroid agonist according to claim 133, or at least one antibody drug conjugate (ADC) comprising said steroid agonist according to claim 133, or a steroid-linker comprising said steroid agonist according to claim 133, and a pharmaceutically acceptable carrier; which is administrable via an injection route, optionally intravenous, intramuscular, intrathecal, or subcutaneous; or optionally is subcutaneously administrable.

142. A device comprising therapeutically effective amount of at least one antibody drug conjugate (ADC) or steroid agonist according to claim 133, at least one steroid-linker comprising said steroid agonist according to claim 133, or at least one antibody drug conjugate (ADC) comprising said steroid agonist according to claim 133, or a steroid-linker comprising said steroid agonist according to claim 133, that provides for subcutaneous administration selected from the group consisting of a syringe, an injection device, an infusion pump, an injector pen, a needleless device, an autoinjector, and a subcutaneous patch delivery system; which optionally delivers to a patient a fixed dose of the glucocorticoid receptor agonist, or a functional derivative thereof; and/or optionally A kit comprising the device, which optionally further comprises instructions informing the patient how to administer the ADC composition comprised therein and the dosing regimen.

143. A method of treatment or prophylaxis which comprises the administration of a therapeutically effective amount of at least one antibody drug conjugate (ADC) or steroid agonist according to claim 133, at least one steroid-linker comprising said steroid agonist according to claim 133, or at least one antibody drug conjugate (ADC) comprising said steroid agonist according to claim 133, or a steroid-linker comprising said steroid agonist according to claim 133, wherein said composition optionally is in a device; optionally for:
(i) treatment of allergy, autoimmunity, transplant, gene therapy, inflammation, GVHD or sepsis, or to treat or prevent inflammatory, autoimmune, or allergic side effects associated with any of the foregoing conditions in a human subject, optionally wherein the inflammation is associated with cancer, or an infection, optionally a viral or bacterial infection, or
(ii) treatment of a condition selected from rheumatoid arthritis, juvenile idiopathic arthritis, psoriatic arthritis, ankylosing spondylitis, adult Crohn's disease, pediatric Crohn's disease, ulcerative colitis, plaque psoriasis, hidradenitis suppurativa, uveitis, Bechet's disease, a spondyloarthropathy, or psoriasis; or
(iii) treatment of one or more of the following:
(a) a chronic, acute, episodic allergic, inflammatory or inflammatory condition, e.g., chronic, acute, episodic, remitting/relapsing;
(b) a condition primarily only effectively treatable with high doses of steroids, optionally polymyalgia rheumatica and/or giant cell arteritis, which patient optionally has been treated or is undergoing treatment with high steroid doses;
(c) a condition with a comorbidity limiting steroid use, optionally diabetes mellitis, nonalcoholic steatohepatitis (NASH), morbid obesity avascular necrosis/osteonecrosis (AVN), glaucoma. Steroid-induced hypertension, severe skin fragility, and/or osteoarthritis;
(d) a condition wherein safe long-term treatment agents are available, but wherein several months of induction with high-doses of steroids is desired, optionally AAV, polymyositis, dermamyositis, lupus, inflammatory lung disease, autoimmune hepatitis, inflammatory bowel disease, immune thrombocytopenia, autoimmune hemolytic anemia, gout patients wherein several months of induction with high-doses of steroids is therapeutically warranted;
(e) dermatologic conditions that require short/long-term treatment, optionally of uncertain treatment or duration and/or no effective alternative to steroid administration, optionally Stevens Johnson, other severe drug eruption conditions, conditions involving extensive contact dermatitis, other severe immune-related dermatological conditions such as PG, LCV, Erythroderma and the like;
(f) conditions treated with high-dose corticosteroids for flares/reoccurrences, optionally COPD, asthma, lupus, gout, pseudogout;
(g) immune-related neurologic diseases such as small-fiber neuropathy, MS (subset), chronic inflammatory demyelinating polyneuropathy, myasthenia gravis and the like;
(h) hematological/oncology indications, optionally wherein high doses of steroids would potentially be therapeutically warranted or beneficial;
(i) ophthalmologic conditions, optionally uveitis, iritis, scleritis, and the like;
(j) conditions associated with permanent or very prolonged adrenal insufficiency or secondary adrenal insufficiency, optionally latrogenic Addisonian crisis;
(k) conditions often treated with long term, low dose steroids, optionally lupus, RA, psA, vasculitis, and the like; and special classes of patients such as pregnant/breast-feeding women, pediatric patients optionally those with growth impairment or cataracts; or
(iv) treatment in combination with another active agent; optionally an immunomodulatory antibody or fusion protein which is selected from immmunoinhibitory antibodies or fusion proteins targeting one or more of CTLA4, PD-1, PDL-1, LAG-3, TIM-3, BTLA, B7-H4, B7-H3, VISTA, and/or agonistic antibodies or fusion protein targeting one or more of CD40, CD137, OX40, GITR, CD27, CD28 or ICOS; or
any combination of the foregoing.

144. A method of treatment or prophylaxis which comprises the administration of a therapeutically effective amount of at least one antibody drug conjugate (ADC) or steroid agonist according to claim 133, at least one steroid-linker comprising said steroid agonist according to claim 133, or at least one antibody drug conjugate (ADC) comprising said steroid agonist according to claim 133, or a steroid-linker comprising said steroid agonist according to claim 133, wherein immune cells from a patient or donor are contacted ex vivo with said at least one antibody drug conjugate (ADC) or steroid agonist according to claim 133, at least one steroid-linker comprising said steroid agonist according to claim 133, or at least one antibody drug conjugate (ADC) comprising said steroid agonist according to claim 133, or at least one antibody drug conjugate (ADC) comprising said steroid-linker comprising said steroid agonist according to claim 133.

145. The ADC of claim 135, wherein
(i) the linker is a positive, negative or neutral charged cleavable peptide, optionally esterase cleavable;
(ii) the drug antibody ratio ranges from 1-12:1 or 1-10:1;
(iii) the drug antibody ratio ranges from 2-8:1, 4-8:1, or 6-8:1;
(iv) the drug antibody ratio the drug antibody ratio is 4:1 (n=4), 6:1 (n=6), 8:1 (n=8), 10:1 (n=10), 12:1 (n=12), or n is 12 or greater and ranges from 12-50;
(v) it internalizes one or more of activated or non-activated monocytes, myeloid cells, B cells, NK cells, T cells, CD4 T cells, CD8 T cells, Tregs, eoinophils, dendritic cells, mast cells, macrophages and neutrophils;
(vi) it does not appreciably internalize activated or non-activated B cells;
(vii) when administered to a subject in need thereof promotes the efficacy and/or reduces adverse side effects associated with the glucocorticoid receptor agonist, compared to the same dosage of anti-inflammatory agent administered in naked (non-conjugated) form;
(viii) the glucocorticoid receptor agonist therein is conjugated to the antibody or antigen-binding fragment via the interchain disulfides;
(ix) it comprises an esterase sensitive linker;
(x) it comprises a the cleavable linker is susceptible to one or more of acid-induced cleavage, photo-induced cleavage, peptidase-induced cleavage, esterase-induced cleavage, and disulfide bond cleavage;
(xi) it comprises a non-cleavable linker that is substantially resistant to one or more of acid-induced cleavage, photo-induced cleavage, peptidase-induced cleavage, esterase-induced cleavage and disulfide bond cleavage;
(xii) it comprises an anti-VISTA antigen binding fragment comprised in the ADC which comprises a Fab, F(ab′)2, or scFv antibody fragment;
or any combination of (i) to (xii).

146. At least one antibody drug conjugate (ADC) or steroid agonist according to claim 133, at least one steroid-linker comprising said steroid agonist according to claim 133, or at least one antibody drug conjugate (ADC) comprising said steroid agonist according to claim 133, or a steroid-linker comprising said steroid agonist according to claim 133, for
(i) treatment of allergy, autoimmunity, transplant, gene therapy, inflammation, GVHD or sepsis, or to treat or prevent inflammatory, autoimmune, or allergic side effects associated with any of the foregoing conditions in a human subject; optionally wherein the patient comprises a condition selected from rheumatoid arthritis, juvenile idiopathic arthritis, psoriatic arthritis, ankylosing spondylitis, adult Crohn's disease, pediatric Crohn's disease, ulcerative colitis, plaque psoriasis, hidradenitis suppurativa, uveitis, Bechet's disease, a spondyloarthropathy, or psoriasis; or
(ii) treating a patient who comprises one or more of the following:
(a) a condition primarily only effectively treatable with high doses of steroids, optionally polymyalgia rheumatica and/or giant cell arteritis, which patient optionally has been treated or is undergoing treatment with high steroid doses;
(b) a condition with a comorbidity limiting steroid use, optionally diabetes mellitis, nonalcoholic steatohepatitis (NASH), morbid obesity avascular necrosis/osteonecrosis (AVN), glaucoma. Steroid-induced hypertension, severe skin fragility, and/or osteoarthritis;
(c) a condition wherein safe long-term treatment agents are available, but wherein several months of induction with high-doses of steroids is desired, optionally AAV, polymyositis, dermamyositis, lupus, inflammatory lung disease, autoimmune hepatitis, inflammatory bowel disease, immune thrombocytopenia, autoimmune hemolytic anemia, gout patients wherein several months of induction with high-doses of steroids is therapeutically warranted;
(d) dermatologic conditions that require short/long-term treatment, optionally of uncertain treatment or duration and/or no effective alternative to steroid administration, optionally Stevens Johnson, other severe drug eruption conditions, conditions involving extensive contact dermatitis, other severe immune-related dermatological conditions such as PG, LCV, Erythroderma and the like;
(e) conditions treated with high-dose corticosteroids for flares/reoccurrences, optionally COPD, asthma, lupus, gout, pseudogout;
(f) immune-related neurologic diseases such as small-fiber neuropathy, MS (subset), chronic inflammatory demyelinating polyneuropathy, myasthenia gravis and the like;
(g) hematological/oncology indications, optionally wherein high doses of steroids would potentially be therapeutically warranted or beneficial;
(h) ophthalmologic conditions, optionally uveitis, iritis, scleritis, and the like;
(i) conditions associated with permanent or very prolonged adrenal insufficiency or secondary adrenal insufficiency, optionally latrogenic Addisonian crisis;
(j) conditions often treated with long term, low dose steroids, optionally lupus, RA, psA, vasculitis, and the like; and
(k) special classes of patients such as pregnant/breast-feeding women, pediatric patients optionally those with growth impairment or cataracts;
(iii) treating a patient who is further being treated with another active agent;
(iv) treating a patient who is further being treated with an immunomodulatory antibody or fusion protein which is selected from immmunoinhibitory antibodies or fusion proteins targeting one or more of CTLA4, PD-1, PDL-1, LAG-3, TIM-3, BTLA, B7-H4, B7-H3, VISTA, and/or agonistic antibodies or fusion protein targeting one or more of CD40, CD137, OX40, GITR, CD27, CD28 or ICOS;
(v) treatment or prophylaxis of Acute or chronic inflammation and autoimmune and inflammatory indications associated therewith wherein the conditions optionally include Severe asthma, Giant cell arteritis, ANKA vasculitis and IBD (Colitis and Crohns);
(vi) treatment or prophylaxis of a condition selected from rheumatoid arthritis, juvenile idiopathic arthritis, psoriatic arthritis, ankylosing spondylitis, adult Crohn's disease, pediatric Crohn's disease, ulcerative colitis, plaque psoriasis, hidradenitis suppurativa, uveitis, Bechet's disease, a spondyloarthropathy, or psoriasis;
(vii) effecting ex vivo or in vivo internalization of a glucocorticosteroid into one or more of T cells, CD4 T cells, CD8 T cells, Tregs, NK cells, B cells, Neutrophils, monocytes, myeloid cells, Dendritic cells, eosinophils, mast cells, and macrophages; optionally ex vivo, by contacting the ADC with a purified or enriched composition comprising immune cells or comprising a specific type or types of immune cells selected from B cells, T cells, CD4 T cells, CD8 T cells, Tregs, NK cells, Neutrophils, monocytes, myeloid cells, Dendritic cells, eosinophils, mast cells, and macrophages which is introduced into a patient in need thereof; or
(viii) treating an inflammatory or autoimmune or allergic condition involving one or more of any of B cells, T cells, Tregs, NK cells, Neutrophils, monocytes, myeloid cells, dendritic cells, eosinophils, mast cells, and macrophages; or a combination of any of the foregoing.

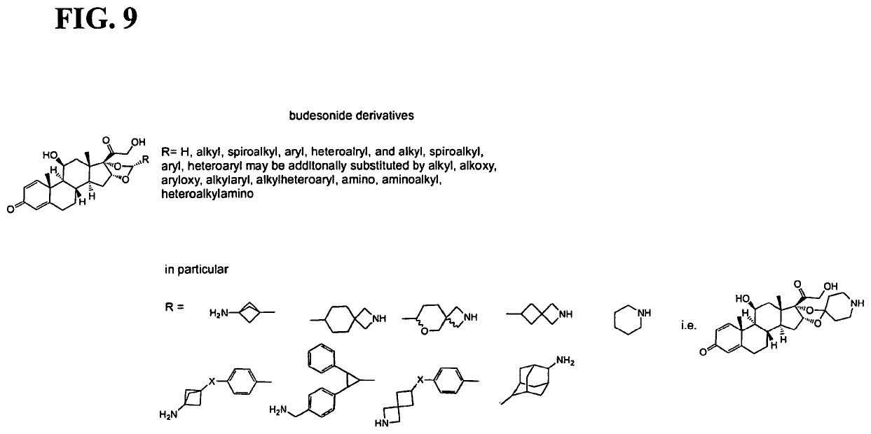

147. At least one antibody drug conjugate (ADC) or steroid agonist according to claim 133, at least one steroid-linker comprising said steroid agonist according to claim 133, or at least one antibody drug conjugate (ADC) comprising said steroid agonist according to claim 133, or a steroid-linker comprising said steroid agonist according to claim 133, wherein the glucocorticoid agonist does not consist of: 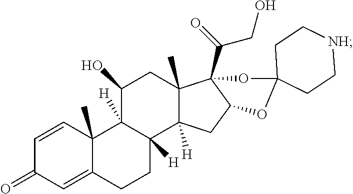 [SMILES: C[C@]12C=CC(=O)C=C1CC[C@@H]1[C@@H]2[C@@H](O)C[C@@]2(C)[C@H]1C[C@H]1OC3(CCNCC3)O[C@]12C(=O)CO]
or a compound depicted in FIG. 9 and/or having the structure below: 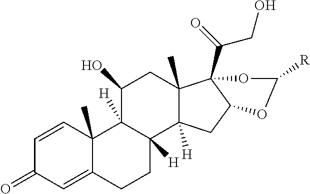
wherein:
R═H, or [chemical structure]
